(19) United States
(12) Patent Application Publication
(10) Pub. No.: US 2024/0309370 A1
(43) Pub. Date: Sep. 19, 2024

(54) METABOLIC DISORDER-ASSOCIATED TARGET GENE IRNA COMPOSITIONS AND METHODS OF USE THEREOF

(71) Applicant: Alnylam Pharmaceuticals, Inc., Cambridge, MA (US)

(72) Inventors: Aimee M. Deaton, Somerville, MA (US); Jeffrey Zuber, Somerville, MA (US)

(21) Appl. No.: 18/403,912

(22) Filed: Jan. 4, 2024

Related U.S. Application Data
(63) Continuation of application No. PCT/US22/37658, filed on Jul. 20, 2022.
(60) Provisional application No. 63/223,995, filed on Jul. 21, 2021.
(60) Provisional application No. 63/278,126, filed on Nov. 11, 2021.
(60) Provisional application No. 63/285,143, filed on Dec. 2, 2021.
(60) Provisional application No. 63/287,578, filed on Dec. 9, 2021.
(60) Provisional application No. 63/321,799, filed on Mar. 21, 2022.
(60) Provisional application No. 63/323,543, filed on Mar. 25, 2022.

(51) Int. Cl. C12N 15/113

(52) U.S. Cl. CPC C12N 15/113; C12N 15/1137; C12N 15/1138; C12Y 301/04017; C12N 2310/14; C12N 2310/3125; C12N 2310/315; C12N 2310/321; C12N 2310/322; C12N 2310/3231; C12N 2310/344; C12N 2310/351

(57) ABSTRACT

The present invention relates to RNAi agents, e.g., double stranded RNA (dsRNA) agents, targeting a metabolic disorder-associated target gene, e.g., inhibin subunit beta E (INHBE), activin A receptor type 1C (ACVR1C), perilipin-1 (PLIN1), phosphodiesterase 3B (PDE3B), or inhibin subunit beta C (INHBC) gene. The invention also relates to methods of using such RNAi agents to inhibit expression of a metabolic disorder-associated target gene and to methods of preventing and treating a metabolic disorder, e.g., metabolic syndrome.

### RELATED APPLICATIONS

This application is a 35 § U.S.C. 111(a) continuation application which claims the benefit of priority to PCT/US2022/037658, filed on Jul. 20, 2022, which, in turn, claims the benefit of priority to U.S. Provisional Application No. 63/223,995, filed on Jul. 21, 2021, U.S. Provisional Application No. 63/278,126, filed on Nov. 11, 2021, U.S. Provisional Application No. 63/285,143, filed on Dec. 2, 2021, U.S. Provisional Application No. 63/287,578, filed on Dec. 9, 2021, U.S. Provisional Application No. 63/321,799, filed on Mar. 21, 2022, and U.S. Provisional Application No. 63/323,543, filed on Mar. 25, 2022. The entire contents of each of the foregoing applications are incorporated herein by reference.

### SEQUENCE LISTING

The instant application contains a Sequence Listing which has been submitted electronically in XML file format via Patent Center and is hereby incorporated by reference in its entirety. Said XML copy, created on Jan. 3, 2024, is named 121301_16307_SL.xml and is 58,454,450 bytes in size.

### BACKGROUND OF THE INVENTION

With the successful conquest of many infectious diseases in most of the world, non-communicable diseases, metabolic disorders in particular, have become a major health hazard of the modern world. The increase in consumption of high calorie-low fiber fast food and the decrease in physical activity due to mechanized transportations and sedentary lifestyle have resulted in the spread of metabolic disorders such as metabolic syndrome, type 2 diabetes, hypertension, cardiovascular diseases, stroke, and other disabilities. Indeed, the occurrences of subjects with a metabolic disorder, such as, metabolic syndrome, who have a number of health conditions placing them at higher risk for heart disease, diabetes, stroke, and other diseases have increased in the recent years.

Current treatments for disorders of metabolic disorders include lifestyle changes, dieting, exercise and treatment with agents, such as lipid lowering agents, e.g., statins, and other drugs. However, these therapies and treatments are often limited by compliance, are not always effective, result in side effects, and result in drug-drug interactions. Accordingly, there is a need in the art for alternative treatments for subjects having metabolic disorders, such as metabolic syndrome and related diseases, e.g., diabetes, hypertension, and cardiovascular disease, such as an agent that can selectively and efficiently silence a metabolic disorder-associated target gene, i.e., inhibin subunit beta E (INHBE), activin A receptor type 1C (ACVR1C), perilipin-1 (PLIN1), phosphodiesterase 3B (PDE3B), or inhibin subunit beta C (INHBC), using the cell's own RNAi machinery that has both high biological activity and in vivo stability, and that can effectively inhibit expression of the metabolic disorder-associated target INHBE gene.

### SUMMARY OF THE INVENTION

The present invention provides iRNA compositions which effect the RNA-induced silencing complex (RISC)-mediated cleavage of RNA transcripts of a gene encoding a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC). The target gene may be within a cell, e.g., a cell within a subject, such as a human subject. The present invention also provides methods of using the iRNA compositions of the invention for inhibiting the expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC), and/or for treating a subject who would benefit from inhibiting or reducing the expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC), e.g., a subject suffering or prone to suffering from a metabolic disorder, e.g., metabolic syndrome, and/or cardiovascular disease.

Accordingly, in an aspect, the invention provides a double stranded ribonucleic acid (dsRNA) agent for inhibiting expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC) in a cell, such as an adipocyte and/or a liver cell, wherein the dsRNA agent comprises a sense strand and an antisense strand forming a double stranded region, wherein the sense strand comprises at least 15, e.g., 15, 16, 17, 18, 19, 20, or 21, contiguous nucleotides differing by no more than 0, 1, 2, or 3 nucleotides from the nucleotide sequence of any one of SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, or 55, and the antisense strand comprises at least 15, e.g., 15, 16, 17, 18, 19, 20, 21, 22, or 23, contiguous nucleotides differing by no more than 0, 1, 2, or 3 nucleotides from the corresponding portion of the nucleotide sequence of any one of SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, or 56.

In another aspect, the present invention provides a double stranded ribonucleic acid (dsRNA) agent for inhibiting expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC) in a cell, such as an adipocyte and/or a liver cell, wherein the dsRNA agent comprises a sense strand and an antisense strand forming a double stranded region, wherein the antisense strand comprises a region of complementarity to an mRNA encoding the target gene, and wherein the region of complementarity comprises at least 15, e.g., 15, 16, 17, 18, 19, 20, 21, 22, or 23, contiguous nucleotides differing by no more than 0, 1, 2, or 3 nucleotides from any one of the antisense nucleotide sequences in any one of Tables 2-17, 19 and 20.

In yet another aspect, the present invention provides a double stranded ribonucleic acid (dsRNA) agent for inhibiting expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC) in a cell, such as an adipocyte and/or a liver cell, wherein the dsRNA agent comprises a sense strand and an antisense strand forming a double stranded region, wherein the sense strand comprises at least 15, e.g., 15, 16, 17, 18, 19, 20, or 21, contiguous nucleotides differing by no more than 0, 1, 2, or 3 nucleotides from any one of the sense nucleotide sequences in any one of Tables 2-17, 19 and 20 and the antisense strand comprises at least 15, e.g., 15, 16, 17, 18, 19, 20, 21, 22, or 23, contiguous nucleotides differing by no more than 0, 1, 2, or 3 nucleotides from any one of the antisense nucleotide sequences in any one of Tables 2-17, 19 and 20. In some embodiments, these dsRNA agents further comprise one or more C22 hydrocarbon chains conjugated to one or more positions, e.g., internal positions, on at least one strand of the dsRNA agent.

In another aspect, the present invention provides a double stranded ribonucleic acid (dsRNA) agent for inhibiting expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC) in a cell, such as an adipocyte and/or a liver cell, wherein the dsRNA agent comprises a sense strand and an antisense strand forming a double stranded region, wherein the sense strand comprises at least 15, e.g., 15, 16, 17, 18, 19, 20, or 21, contiguous nucleotides differing by no more than 0, 1, 2, or 3 nucleotides from any one of the sense nucleotide sequences in any one of Tables 2-17, 19 and 20 and the antisense strand comprises at least 15, e.g., 15, 16, 17, 18, 19, 20, 21, 22, or 23, contiguous nucleotides differing by no more than 0, 1, 2, or 3 nucleotides from any one of the antisense nucleotide sequences in any one of Tables 2-17, 19 and 20. In some embodiments, these dsRNA agents further comprise one or more GalNAcligands conjugated to at least one strand of the dsRNA agent, e.g., through a bivalent or trivalent branched linker.

In one embodiment, the dsRNA agent comprises a sense strand comprising a contiguous nucleotide sequence which has at least 85%, e.g., 85%, 90%, 91%, 92%, 93%, 94%, 95%, 96%, 97%, 98%, 99%, or 100%, nucleotide sequence identity over its entire length to any one of the nucleotide sequences of the sense strands in any one of Tables 2-17, 19 and 20 and an antisense strand comprising a contiguous nucleotide sequence which has at least 85%, e.g., 85%, 90%, 91%, 92%, 93%, 94%, 95%, 96%, 97%, 98%, 99%, or 100%, nucleotide sequence identity over its entire length to any one of the nucleotide sequences of the antisense strands in any one of Tables 2-17, 19 and 20.

In one embodiment, the dsRNA agent comprises a sense strand comprising at least 15, e.g., 15, 16, 17, 18, 19, 20, or 21, contiguous nucleotides differing by no more than three nucleotides from any one of the nucleotide sequences of the sense strands in any one of Tables 2-17, 19 and 20 and an antisense strand comprising at least 15, e.g., 15, 16, 17, 18, 19, 20, 21, 22, or 23 contiguous nucleotides differing by no more than three nucleotides from any one of the nucleotide sequences of the antisense strands in any one of Tables 2-17, 19 and 20.

In one embodiment, the dsRNA agent comprises a sense strand comprising at least 15, e.g., 15, 16, 17, 18, 19, 20, or 21, contiguous nucleotides differing by no more than two nucleotides from any one of the nucleotide sequences of the sense strands in any one of Tables 2-17, 19 and 20 and an antisense strand comprising at least 15, e.g., 15, 16, 17, 18, 19, 20, 21, or 23 contiguous nucleotides differing by no more than two nucleotides from any one of the nucleotide sequences of the antisense strands in any one of Tables 2-17, 19 and 20.

In one embodiment, the dsRNA agent comprises a sense strand comprising at least 15, e.g., 15, 16, 17, 18, 19, 20, or 21, contiguous nucleotides differing by no more than one nucleotide from any one of the nucleotide sequences of the sense strands in any one of Tables 2-17, 19 and 20 and an antisense strand comprising at least 15, e.g., 15, 16, 17, 18, 19, 20, 21, 22, or 23 contiguous nucleotides differing by no more than one nucleotide from any one of the nucleotide sequences of the antisense strands in any one of Tables 2-17, 19 and 20.

In one embodiment, the dsRNA agent comprises a sense strand comprising or consisting of a nucleotide sequence selected from the group consisting of any one of the nucleotide sequences of the sense strands in any one of Tables 2-17, 19 and 20 and an antisense strand comprising or consisting of a nucleotide sequence selected from the group consisting of any one of the nucleotide sequences of the antisense strands in any one of Tables 2-17, 19 and 20.

In one embodiment, the target gene is INHBE.

In one embodiment, the target gene is ACVR1C.

In one embodiment, the target gene is PLIN1.

In one embodiment, the target gene is PDE3B.

In one embodiment, the target gene is INHBC.

In one aspect, the present invention provides a double stranded ribonucleic acid (dsRNA) agent for inhibiting expression of inhibin subunit beta E (INHBE) in a cell, such as an adipocyte and/or a liver cell, wherein said dsRNA agent comprises a sense strand and an antisense strand forming a double stranded region, wherein the sense strand comprises at least 15, e.g., 15, 16, 17, 18, 19, 20, or 21, contiguous nucleotides differing by no more than 0, 1, 2, or 3 nucleotides from any one of the nucleotide sequences of nucleotides 400-422, 410-432, 518-540, 519-541, 640-662, 1430-1452, 1863-1885, or 1864-1886 of SEQ ID NO: 1, and the antisense strand comprises at least 15, e.g., 15, 16, 17, 18, 19, 20, 21, 22, or 23, differing by no more than 0, 1, 2, or 3 nucleotides from the corresponding nucleotide sequence of SEQ ID NO:2.

In one aspect, the present invention provides a double stranded ribonucleic acid (dsRNA) agent for inhibiting expression of inhibin subunit beta E (INHBE) in a cell, such as an adipocyte and/or a liver cell, wherein said dsRNA agent comprises a sense strand and an antisense strand forming a double stranded region, wherein the sense strand comprises at least 15, e.g., 15, 16, 17, 18, 19, 20, or 21, contiguous nucleotides differing by no more than 0, 1, 2, or 3 nucleotides from any one of the nucleotide sequence of nucleotides 400-422, 410-432, 518-540, 519-541, 640-662, 1430-1452, 1863-1885, or 1864-1886 of SEQ ID NO: 1, and the antisense strand comprises at least 15, e.g., 15, 16, 17, 18, 19, 20, 21, 22, or 23, contiguous nucleotides differing by no more than 0, 1, 2, or 3 nucleotides from the corresponding nucleotide sequence of SEQ ID NO:2. In some embodiments, these dsRNA agents further comprise one or more C22 hydrocarbon chains conjugated to one or more positions, e.g., internal positions, on at least one strand of the dsRNA agent.

In one aspect, the present invention provides a double stranded ribonucleic acid (dsRNA) agent for inhibiting expression of inhibin subunit beta E (INHBE) in a cell, such as an adipocyte and/or a liver cell, wherein said dsRNA agent comprises a sense strand and an antisense strand forming a double stranded region, wherein the sense strand comprises at least 15, e.g., 15, 16, 17, 18, 19, 20, or 21, contiguous nucleotides differing by no more than 0, 1, 2, or 3 nucleotides from any one of the nucleotide sequence of nucleotides 400-422, 410-432, 518-540, 519-541, 640-662, 1430-1452, 1863-1885, or 1864-1886 of SEQ ID NO: 1, and the antisense strand comprises at least 15, e.g., 15, 16, 17, 18, 19, 20, 21, 22, or 23, contiguous nucleotides differing by no more than 0, 1, 2, or 3 nucleotides from the corresponding nucleotide sequence of SEQ ID NO:2. In some embodiments, these dsRNA agents further comprise one or more GalNAcligands conjugated to at least one strand of the dsRNA agent, e.g., through a bivalent or trivalent branched linker.

In some embodiments, the antisense strand comprises at least 15, e.g., 15, 16, 17, 18, 19, 20, 21, 22, or 23, contiguous nucleotides differing by no more than 0, 1, 2, or 3 nucleotides from any one of the antisense strand nucleotide sequences of a duplex selected from the group consisting of AD-1706583, AD-1711744, AD-1706593, AD-1708473, AD-1706662, AD-1706761, AD-1707306, AD-1707639, AD-1707640.

In some embodiments, the sense and the antisense strand comprise at least 15, e.g., 15, 16, 17, 18, 19, 20, 21, 22, or 23, contiguous nucleotides differing by no more than 0, 1, 2, or 3 nucleotides from any one of the sense and the antisense strand nucleotide sequences of a duplex selected from the group consisting of AD-1706583, AD-1711744, AD-1706593, AD-1708473, AD-1706662, AD-1706761, AD-1707306, AD-1707639, AD-1707640.

In some embodiments, the antisense strand comprises at least 15, e.g., 15, 16, 17, 18, 19, 20, 21, 22, or 23, contiguous nucleotides differing by no more than 0, 1, 2, or 3 nucleotides from any one of the antisense strand nucleotide sequences selected from the group consisting of

(SEQ ID NO: 57)
(a) 5′-AGUUAUTCUGGGACGACUGGUCA-3′;
(SEQ ID NO: 58)
(b) 5′-AGUUAUTCUGGGACGACUGGUCU-3′;
(SEQ ID NO: 59)
(c) 5′-ATGGAGGAUGAGUUAUUCUGGGA-3′;
(SEQ ID NO: 60)
(d) 5′-AUGAAGTGGAGUCUGUGACAGUA-3′;
(SEQ ID NO: 61)
(e) 5′-ACUGAAGUGGAGUCUGUGACAGU-3′;
(SEQ ID NO: 62)
(f) 5′-ACGGAAGAUCCTCAAGCAAAGAG-3′;
(SEQ ID NO: 63)
(g) 5′-ACAGACAAGAAAGUGCCCAUUUG-3′;
(SEQ ID NO: 64)
(h) 5′-AAGAAAGUAUAAAUGCUUGUCUC-3′;
and
(SEQ ID NO: 65)
(i) 5′-AAAGAAAGUAUAAAUGCUUGUCU-3′.

In some embodiments, the sense strand comprises at least 15, e.g., 15, 16, 17, 18, 19, 20, or 21, contiguous nucleotides differing by no more than 0, 1, 2, or 3 nucleotides and the antisense strand comprises at least 15, e.g., 15, 16, 17, 18, 19, 20, 21, 22, or 23, contiguous nucleotides differing by no more than 0, 1, 2, or 3 nucleotides from any one of the sense and antisense strand nucleotide sequences selected from the group consisting of

(SEQ ID NO: 66)
(a) 5′-ACCAGUCGUCCCAGAAUAACU-3′
and
(SEQ ID NO: 57)
5′-AGUUAUTCUGGGACGACUGGUCA-3′;
(SEQ ID NO: 66)
(b) 5′-ACCAGUCGUCCCAGAAUAACU-3′
and
(SEQ ID NO: 58)
5′-AGUUAUTCUGGGACGACUGGUCU-3′;
(SEQ ID NO: 67)
(c) 5′-CCAGAAUAACUCAUCCUCCAU-3′
and
(SEQ ID NO: 59)
5′-ATGGAGGAUGAGUUAUUCUGGGA-3′;
(SEQ ID NO: 68)
(d) 5′-CUGUCACAGACUCCACUUCAU-3′
and
(SEQ ID NO: 60)
5′-AUGAAGTGGAGUCUGUGACAGUA-3′;
(SEQ ID NO: 69)
(e) 5′-UGUCACAGACUCCACUUCAGU-3′
and
(SEQ ID NO: 61)
5′-ACUGAAGUGGAGUCUGUGACAGU-3′;
(SEQ ID NO: 70)
(f) 5′-CUUUGCUUGAGGAUCUUCCGU-3′
and
(SEQ ID NO: 62)
5′-ACGGAAGAUCCTCAAGCAAAGAG-3′;
(SEQ ID NO: 71)
(g) 5′-AAUGGGCACUUUCUUGUCUGU-3′
and
(SEQ ID NO: 63)
5′-ACAGACAAGAAAGUGCCCAUUUG-3′;
(SEQ ID NO: 72)
(h) 5′-GACAAGCAUUUAUACUUUCUU-3′
and
(SEQ ID NO: 64)
5′-AAGAAAGUAUAAAUGCUUGUCUC-3′;
and
(SEQ ID NO: 73)
(i) 5′-ACAAGCAUUUAUACUUUCUUU-3′
and
(SEQ ID NO: 65)
5′-AAAGAAAGUAUAAAUGCUUGUCU-3′.

In one embodiment, the dsRNA agent comprises at least one modified nucleotide.

In one embodiment, substantially all of the nucleotides of the sense strand are modified nucleotides; substantially all of the nucleotides of the antisense strand are modified nucleotides; or substantially all of the nucleotides of the sense strand and substantially all of the nucleotides of the antisense strand are modified nucleotides.

In one embodiment, all of the nucleotides of the sense strand are modified nucleotides; all of the nucleotides of the antisense strand are modified nucleotides; or all of the nucleotides of the sense strand and all of the nucleotides of the antisense strand are modified nucleotides.

In one embodiment, at least one of the modified nucleotides is selected from the group consisting of a deoxy-nucleotide, a 3′-terminal deoxythimidine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a thermally destabilizing nucleotide, a glycol modified nucleotide (GNA), a nucleotide comprising a 2′ phosphate, and a 2-O—(N-methylacetamide) modified nucleotide; and combinations thereof.

In one embodiment, at least one of the modified nucleotides is selected from the group consisting of LNA, HNA, CeNA, 2′-methoxyethyl, 2′-O-alkyl, 2′-O-allyl, 2′-C— allyl, 2′-fluoro, 2′-deoxy, 2′-hydroxyl, and glycol; and combinations thereof.

In one embodiment, at least one of the modified nucleotides is selected from the group consisting of a deoxy-nucleotide, a 2′-O-methyl modified nucleotide, a 2′-fluoro modified nucleotide, a 2′-deoxy-modified nucleotide, a glycol modified nucleotide (GNA), e.g., Ggn, Cgn, Tgn, or Agn, a nucleotide with a 2′ phosphate, e.g., G2p, C2p, A2p or U2p, a nucleotide comprising a phosphorothioate group, and a vinyl-phosphonate nucleotide; and combinations thereof.

In another embodiment, at least one of the modified nucleotides is a nucleotide with a thermally destabilizing nucleotide modification.

In one embodiment, the thermally destabilizing nucleotide modification is selected from the group consisting of an abasic modification; a mismatch with the opposing nucleotide in the duplex; a destabilizing sugar modification, a 2′-deoxy modification, an acyclic nucleotide, an unlocked nucleic acid (UNA), and a glycerol nucleic acid (GNA).

In some embodiments, the modified nucleotide comprises a short sequence of 3′-terminal deoxythimidine nucleotides (dT).

In some embodiments, the dsRNA agents further comprise a phosphate or phosphate mimic at the 5′-end of the antisense strand.

In some embodiments, phosphate mimic is a 5′-vinyl phosphonate (VP).

In some embodiments, the 5′-end of the antisense strand of the dsRNA agent does not contain a 5′-vinyl phosphonate (VP).

In some embodiments, the dsRNA agent further comprises at least one terminal, chiral phosphorus atom.

A site specific, chiral modification to the internucleotide linkage may occur at the 5′ end, 3′ end, or both the 5′ end and 3′ end of a strand. This is being referred to herein as a “terminal” chiral modification. The terminal modification may occur at a 3′ or 5′ terminal position in a terminal region, e.g., at a position on a terminal nucleotide or within the last 2, 3, 4, 5, 6, 7, 8, 9 or 10 nucleotides of a strand. A chiral modification may occur on the sense strand, antisense strand, or both the sense strand and antisense strand. Each of the chiral pure phosphorus atoms may be in either Rp configuration or Sp configuration, and combination thereof. More details regarding chiral modifications and chirally-modified dsRNA agents can be found in PCT/US18/67103, entitled “Chirally-Modified Double-Stranded RNA Agents,” filed Dec. 21, 2018, which is incorporated herein by reference in its entirety.

In some embodiments, the dsRNA agent further comprises a terminal, chiral modification occurring at the first internucleotide linkage at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp configuration or Sp configuration.

In one embodiment, the dsRNA agent further comprises a terminal, chiral modification occurring at the first and second internucleotide linkages at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the dsRNA agent further comprises a terminal, chiral modification occurring at the first, second, and third internucleotide linkages at the 3′ end of the antisense strand, having the linkage phosphorus atom in Sp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the dsRNA agent further comprises a terminal, chiral modification occurring at the first and second internucleotide linkages at the 3′ end of the antisense strand, having the linkage phosphorus atom in Sp configuration; a terminal, chiral modification occurring at the third internucleotide linkages at the 3′ end of the antisense strand, having the linkage phosphorus atom in Rp configuration; a terminal, chiral modification occurring at the first internucleotide linkage at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In one embodiment, the dsRNA agent further comprises a terminal, chiral modification occurring at the first and second internucleotide linkages at the 3′ end of the antisense strand, having the linkage phosphorus atom in Sp configuration; a terminal, chiral modification occurring at the first, and second internucleotide linkages at the 5′ end of the antisense strand, having the linkage phosphorus atom in Rp configuration; and a terminal, chiral modification occurring at the first internucleotide linkage at the 5′ end of the sense strand, having the linkage phosphorus atom in either Rp or Sp configuration.

In some embodiments, the 3′ end of the sense strand is protected via an end cap which is a cyclic group having an amine, said cyclic group being selected from the group consisting of pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl.

In one embodiment, the dsRNA agent further comprises at least one phosphorothioate or methylphosphonate internucleotide linkage.

In one embodiment, the phosphorothioate or methylphosphonate internucleotide linkage is at the 3′-terminus of one strand, e.g., the antisense strand or the sense strand.

In another embodiment, the phosphorothioate or methylphosphonate internucleotide linkage is at the 5′-terminus of one strand, e.g., the antisense strand or the sense strand.

In one embodiment, the phosphorothioate or methylphosphonate internucleotide linkage is at the both the 5′- and 3′-terminus of one strand. In one embodiment, the strand is the antisense strand.

In one embodiment, the base pair at the 1 position of the 5′-end of the antisense strand of the duplex is an AU base pair.

The double stranded region may be 19-30 nucleotide pairs in length; 19-25 nucleotide pairs in length; 19-23 nucleotide pairs in length; 23-27 nucleotide pairs in length; or 21-23 nucleotide pairs in length.

In one embodiment, each strand is independently no more than 30 nucleotides in length.

In one embodiment, the sense strand is 21 nucleotides in length and the antisense strand is 23 nucleotides in length.

The region of complementarity may be at least 17 nucleotides in length; between 19 and 23 nucleotides in length; or 19 nucleotides in length.

In one embodiment, at least one strand comprises a 3′ overhang of at least 1 nucleotide. In another embodiment, at least one strand comprises a 3′ overhang of at least 2 nucleotides.

In some embodiments, one or more C22 hydrocarbon chains is conjugated to one or more internal positions on at least one strand of the dsRNA agent.

In some embodiments, the lipophilicity of the one or more C22 hydrocarbon chain, measured by octanol-water partition coefficient, log K_{ow}, exceeds 0. The lipophilic moiety may possess a log K_{ow} exceeding 1, exceeding 1.5, exceeding 2, exceeding 3, exceeding 4, exceeding 5, or exceeding 10.

In some embodiments, the hydrophobicity of the dsRNA agent, measured by the unbound fraction in the plasma protein binding assay of the dsRNA agent, exceeds 0.2. In one embodiment, the plasma protein binding assay determined is an electrophoretic mobility shift assay (EMSA) using human serum albumin protein. The hydrophobicity of the dsRNA agent, measured by fraction of unbound dsRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of dsRNA/

The C₂₂ hydrocarbon chain may be saturated or unsaturated.

The C₂₂ hydrocarbon chain may be linear or branched

In some embodiments, the internal positions include all positions except the three terminal positions from each end of the at least one strand.

In some embodiments, the internal positions exclude a cleavage site region of the sense strand.

In some embodiments, the internal positions exclude positions 9-12 or positions 11-13, counting from the 5′-end of the sense strand.

In some embodiments, the internal positions exclude a cleavage site region of the antisense strand.

In some embodiments, the internal positions exclude positions 12-14, counting from the 5′-end of the antisense strand.

In some embodiments, the one or more C₂₂ hydrocarbon chains are conjugated to one or more of the following internal positions: positions 4-8 and 13-18 on the sense strand, and positions 6-10 and 15-18 on the antisense strand, counting from the 5′end of each strand.

In some embodiments, the one or more C₂₂ hydrocarbon chains are conjugated to one or more of the following internal positions: positions 5, 6, 7, 15, and 17 on the sense strand, and positions 15 and 17 on the antisense strand, counting from the 5′-end of each strand.

In some embodiments, the one or more C₂₂ hydrocarbon chains are conjugated to position 6 on the sense strand, counting from the 5′-end of the sense strand.

In some embodiments, the one or more C₂₂ hydrocarbon chains is an aliphatic, alicyclic, or polyalicyclic compound, e.g., the one or more C₂₂ hydrocarbon chains contains a functional group selected from the group consisting of hydroxyl, amine, carboxylic acid, sulfonate, phosphate, thiol, azide, and alkyne.

In some embodiments, the one or more C₂₂ hydrocarbon chains is a C22 acid, e.g. the C22 acid is selected from the group consisting of docosanoic acid, 6-octyltetradecanoic acid, 10-hexylhexadecanoic acid, all-cis-7,10,13,16,19-docosapentaenoic acid, all-cis-4,7,10,13,16,19-docosahexaenoic acid, all-cis-13,16-docosadienoic acid, all-cis-7,10,13,16-docosatetraenoic acid, all-cis-4,7,10,13,16-docosapentaenoic acid, and cis-13-docosenoic acid.

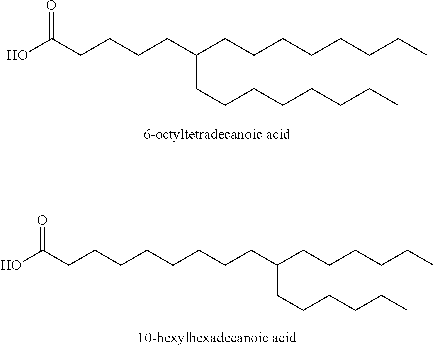 [SMILES: CCCCCCCCC(CCCCCCCC)CCCCC(=O)O ; CCCCCCC(CCCCCC)CCCCCCCCC(=O)O]

In some embodiments, the one or more C₂₂ hydrocarbon chains is a C22 alcohol, e.g., the C22 alcohol is selected from the group consisting of 1-docosanol, 6-octyltetradecan-1-ol, 10-hexylhexadecan-1-ol, cis-13-docosen-1-ol, docosan-9-ol, docosan-2-ol, docosan-10-ol, docosan-11-ol, and cis-4,7,10,13,16,19-docosahexanol.

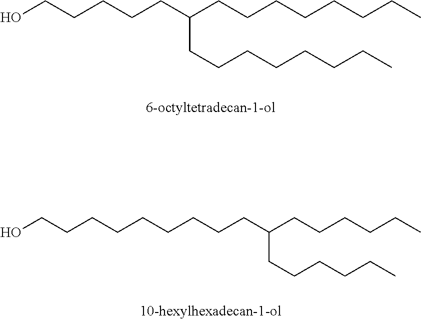 [SMILES: CCCCCCCCC(CCCCCO)CCCCCCCC ; CCCCCCC(CCCCCC)CCCCCCCCCO]

In some embodiments, the one or more C₂₂ hydrocarbon chains is a C22 amide, e.g., the C22 amide is selected from the group consisting of (E)-Docos-4-enamide, (E)-Docos-5-enamide, (Z)-Docos-9-enamide, (E)-Docos-11-enamide, 12-Docosenamide, (Z)-Docos-13-enamide, (Z)—N-Hydroxy-13-docoseneamide, (E)-Docos-14-enamide, 6-cis-Docosenamide, 14-Docosenamide Docos-11-enamide, (4E,13E)-Docosa-4,13-dienamide, and (5E,13E)-Docosa-5,13-dienamide.

The one or more C₂₂ hydrocarbon chains may be conjugated to the dsRNA agent via a direct attachment to the ribosugar of the dsRNA agent. Alternatively, the one or more C₂₂ hydrocarbon chains may be conjugated to the dsRNA agent via a linker or a carrier. In some embodiments, the one or more C₂₂ hydrocarbon chains may be conjugated to the dsRNA agent via internucleotide phosphate linkage.

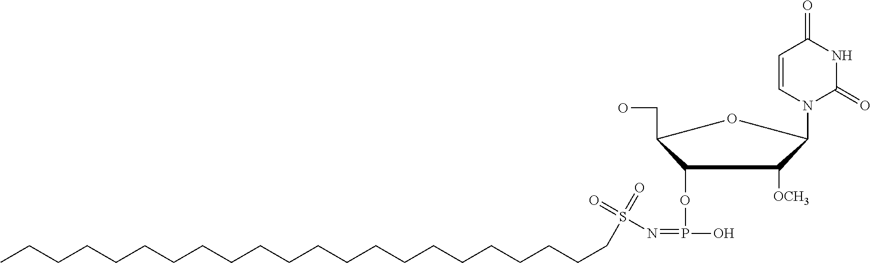

In certain embodiments, the one or more C₂₂ hydrocarbon chains is conjugated to the dsRNA agent via one or more linkers (tethers), e.g., a carrier that replaces one or more nucleotide(s) in the internal position(s).

In some embodiments, the one or more C₂₂ hydrocarbon chains is conjugated to the dsRNA agent via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In some embodiments, at least one of the linkers (tethers) is a redox cleavable linker (such as a reductively cleavable linker; e.g., a disulfide group), an acid cleavable linker (e.g., a hydrazone group, an ester group, an acetal group, or a ketal group), an esterase cleavable linker (e.g., an ester group), a phosphatase cleavable linker (e.g., a phosphate group), or a peptidase cleavable linker (e.g., a peptide bond).

In other embodiments, at least one of the linkers (tethers) is a bio-cleavable linker selected from the group consisting of DNA, RNA, disulfide, amide, functionalized monosaccharides or oligosaccharides of galactosamine, glucosamine, glucose, galactose, mannose, and combinations thereof.

In certain embodiments, the one or more C₂₂ hydrocarbon chains is conjugated to the dsRNA agent via a carrier that replaces one or more nucleotide(s). The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone or a diethanolamine backbone.

In some embodiments, the carrier replaces one or more nucleotide(s) in the internal position(s) of the dsRNA agent.

In some embodiments, the dsRNA agent further comprises a targeting ligand that targets a receptor which mediates delivery to adipose tissue. In one embodiment, the targeting ligand is selected from the group consisting of Angiopep-2, lipoprotein receptor related protein (LRP) ligand, bEnd.3 cell binding ligand, transferrin receptor (TfR) ligand, manose receptor ligand, glucose transporter protein, LDL receptor ligand, trans-retinol, RGD peptide, LDL receptor ligand, CD63 ligand, and carbohydrate based ligand.

In some embodiments, the dsRNA agent further comprises a targeting ligand that targets a liver tissue.

In one embodiment, the targeting ligand is conjugated to the 3′ end of the sense strand of the dsRNA agent.

In some embodiments, the targeting ligand is a carbohydrate-based ligand.

In one embodiment, the targeting ligand is an N-acetylgalactosamine (GalNAc) derivative.

In one embodiment, the targeting ligand is one or more GalNAc derivatives attached through a monovalent, bivalent, or trivalent branched linker.

In one embodiment, the targeting ligand is

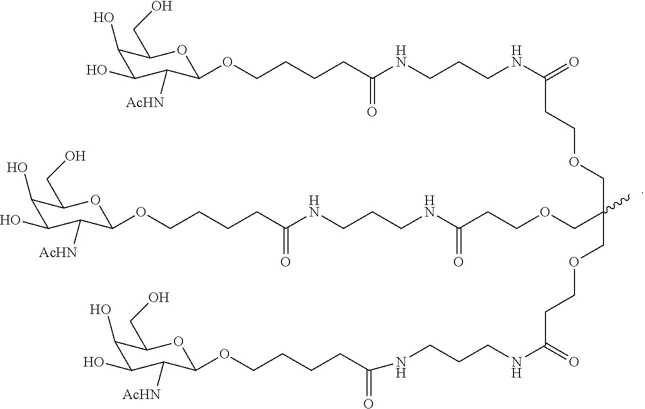 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the dsRNA agent is conjugated to the targeting ligand as shown in the following schematic

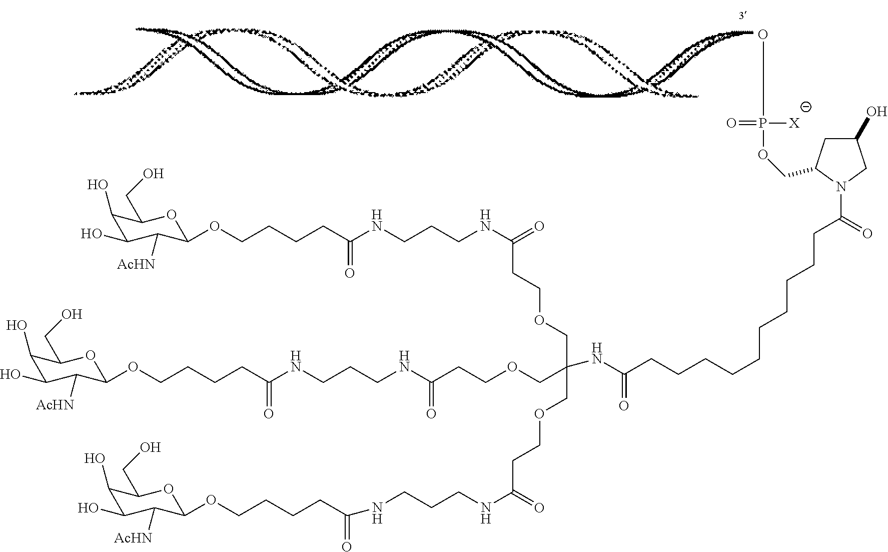

and, wherein X is O or S.

In one embodiment, the X is O.

In some embodiments, the one or more C₂₂ hydrocarbon chains or targeting ligand is conjugated via a bio-cleavable linker selected from the group consisting of DNA, RNA, disulfide, amide, functionalized monosaccharides or oligosaccharides of galactosamine, glucosamine, glucose, galactose, mannose, and combinations thereof.

In one aspect, the present invention provides a double stranded ribonucleic acid (dsRNA) agent for inhibiting expression of inhibin subunit beta E (INHBE), wherein said dsRNA agent comprises a sense strand and an antisense strand forming a double stranded region, wherein the sense strand comprises at least 15, e.g., 15, 16, 17, 18, 19, 20, or 21, contiguous nucleotides differing by no more than 0, 1, 2, 3, or 4 nucleotides from the nucleotide sequence ascscagucgUfCfCfcagaaaawu (SEQ ID NO: 74), wherein the antisense strand comprises at least 15, e.g., 15, 16, 17, 18, 19, 20, 21, 22, or 23, contiguous nucleotides differencing by no more than 0, 1, 2, 3, or 4 nucleotides from the nucleotide sequence asdGsuudAudTcuggdGaCfgacugguscsa (SEQ ID NO:75), wherein a, g, c and u are 2′-O-methyl (2′-OMe) A, G, C, and U; Af, Gf, Cf, and Uf are 2′-fluoro A, G, C and U; dA, dG, dC and dT are 2′-deoxy A, G, C and T; s is a phosphorothioate linkage; and wherein the dsRNA comprises one or more C22 hydrocarbon chains conjugated to one or more internal positions on at least one strand of the dsRNA agent.

In one aspect, the present invention provides a double stranded ribonucleic acid (dsRNA) agent for inhibiting expression of inhibin subunit beta E (INHBE), wherein said dsRNA agent comprises a sense strand and an antisense strand forming a double stranded region, wherein the sense strand comprises at least 15, e.g., 15, 16, 17, 18, 19, 20, or 21, contiguous nucleotides differing by no more than 0, 1, 2, 3, or 4 nucleotides from the nucleotide sequence ascscagucgUfCfCfcagaaaawu (SEQ ID NO: 74), wherein the antisense strand comprises at least 15, e.g., 15, 16, 17, 18, 19, 20, 21, 22, or 23, contiguous nucleotides differencing by no more than 0, 1, 2, 3, or 4 nucleotides from the nucleotide sequence asdGsuudAudTcuggdGaCfgacugguscsu (SEQ ID NO: 76), wherein a, g, c and u are 2′-O-methyl (2′-OMe) A, G, C, and U; Af, Gf, Cf, and Uf are 2′-fluoro A, G, C and U; dA, dG, dC and dT are 2′-deoxy A, G, C and T; s is a phosphorothioate linkage; and wherein the dsRNA comprises one or more C22 hydrocarbon chains conjugated to one or more internal positions on at least one strand of the dsRNA agent.

In one aspect, the present invention provides a double stranded ribonucleic acid (dsRNA) agent for inhibiting expression of inhibin subunit beta E (INHBE), wherein said dsRNA agent comprises a sense strand and an antisense strand forming a double stranded region, wherein the sense strand comprises at least 15, e.g., 15, 16, 17, 18, 19, 20, or 21, contiguous nucleotides differing by no more than 0, 1, 2, 3, or 4 nucleotides from the nucleotide sequence cscsagaauaAfCfUfcauccuccau (SEQ ID NO:77), wherein the antisense strand comprises at least 15, e.g., 15, 16, 17, 18, 19, 20, 21, 22, or 23, contiguous nucleotides differencing by no more than 0, 1, 2, 3, or 4 nucleotides from the nucleotide sequence asdTsggdAgdGaugadGuUfauucuggsgsa (SEQ ID NO: 78), wherein a, g, c and u are 2′-O-methyl (2′-OMe) A, G, C, and U; Af, Gf, Cf, and Uf are 2′-fluoro A, G, C and U; dA, dG, dC and dT are 2′-deoxy A, G, C and T; s is a phosphorothioate linkage; and wherein the dsRNA comprises one or more C22 hydrocarbon chains conjugated to one or more internal positions on at least one strand of the dsRNA agent.

In one aspect, the present invention provides a double stranded ribonucleic acid (dsRNA) agent for inhibiting expression of inhibin subunit beta E (INHBE), wherein said dsRNA agent comprises a sense strand and an antisense strand forming a double stranded region, wherein the sense strand comprises at least 15, e.g., 15, 16, 17, 18, 19, 20, or 21, contiguous nucleotides differing by no more than 0, 1, 2, 3, or 4 nucleotides from the nucleotide sequence csusgucaCfaGfAftfuccacuucau (SEQ ID NO: 79), wherein the antisense strand comprises at least 15, e.g., 15, 16, 17, 18, 19, 20, 21, 22, or 23, contiguous nucleotides differencing by no more than 0, 1, 2, 3, or 4 nucleotides from the nucleotide sequence asUfsgadAg(Tgn)ggagucUfgUfgacagsusa (SEQ ID NO: 80), wherein a, g, c and u are 2′-O-methyl (2′-OMe) A, G, C, and U; Af, Gf, Cf, and Uf are 2′-fluoro A, G, C and U; dA, dG, dC and dT are 2′-deoxy A, G, C and T; Tgn is thymidine-glycol nucleic acid (GNA) S-isomer; s is a phosphorothioate linkage; and wherein the dsRNA comprises one or more C22 hydrocarbon chains conjugated to one or more internal positions on at least one strand of the dsRNA agent.

In one aspect, the present invention provides a double stranded ribonucleic acid (dsRNA) agent for inhibiting expression of inhibin subunit beta E (INHBE), wherein said dsRNA agent comprises a sense strand and an antisense strand forming a double stranded region, wherein the sense strand comprises at least 15, e.g., 15, 16, 17, 18, 19, 20, or 21, contiguous nucleotides differing by no more than 0, 1, 2, 3, or 4 nucleotides from the nucleotide sequence usgsucacagAfCfUfccacuucagu (SEQ ID NO: 81), wherein the antisense strand comprises at least 15, e.g., 15, 16, 17, 18, 19, 20, 21, 22, or 23, contiguous nucleotides differencing by no more than 0, 1, 2, 3, or 4 nucleotides from the nucleotide sequence asdCsugdAadGuggadGuCfugugacasgsu (SEQ ID NO: 82), wherein a, g, c and u are 2′-O-methyl (2′-OMe) A, G, C, and U; Af, Gf, Cf, and Uf are 2′-fluoro A, G, C and U; dA, dG, dC and dT are 2′-deoxy A, G, C and T; s is a phosphorothioate linkage; and wherein the dsRNA comprises one or more C22 hydrocarbon chains conjugated to one or more internal positions on at least one strand of the dsRNA agent.

In one aspect, the present invention provides a double stranded ribonucleic acid (dsRNA) agent for inhibiting expression of inhibin subunit beta E (INHBE), wherein said dsRNA agent comprises a sense strand and an antisense strand forming a double stranded region, wherein the sense strand comprises at least 15, e.g., 15, 16, 17, 18, 19, 20, or 21, contiguous nucleotides differing by no more than 0, 1, 2, 3, or 4 nucleotides from the nucleotide sequence csusuugcuuGfAfGfgaucuuccgu (SEQ ID NO: 83), wherein the antisense strand comprises at least 15, e.g., 15, 16, 17, 18, 19, 20, 21, 22, or 23, contiguous nucleotides differencing by no more than 0, 1, 2, 3, or 4 nucleotides from the nucleotide sequence asdCsggdAadGauccdTcAfagcaaagsasg (SEQ ID NO: 84), wherein a, g, c and u are 2′-O-methyl (2′-OMe) A, G, C, and U; Af, Gf, Cf, and Uf are 2′-fluoro A, G, C and U; dA, dG, dC and dT are 2′-deoxy A, G, C and T; s is a phosphorothioate linkage; and wherein the dsRNA comprises one or more C22 hydrocarbon chains conjugated to one or more internal positions on at least one strand of the dsRNA agent.

In one aspect, the present invention provides a double stranded ribonucleic acid (dsRNA) agent for inhibiting expression of inhibin subunit beta E (INHBE), wherein said dsRNA agent comprises a sense strand and an antisense strand forming a double stranded region, wherein the sense strand comprises at least 15, e.g., 15, 16, 17, 18, 19, 20, or 21, contiguous nucleotides differing by no more than 0, 1, 2, 3, or 4 nucleotides from the nucleotide sequence asasugggcaCfUfUfucuugucugu (SEQ ID NO: 85), wherein the antisense strand comprises at least 15, e.g., 15, 16, 17, 18, 19, 20, 21, 22, or 23, contiguous nucleotides differencing by no more than 0, 1, 2, 3, or 4 nucleotides from the nucleotide sequence asdCsagdAcdAagaadAgUfgcccauususg (SEQ ID NO: 86), wherein a, g, c and u are 2′-O-methyl (2′-OMe) A, G, C, and U; Af, Gf, Cf, and Uf are 2′-fluoro A, G, C and U; dA, dG, dC and dT are 2′-deoxy A, G, C and T; s is a phosphorothioate linkage; and wherein the dsRNA comprises one or more C22 hydrocarbon chains conjugated to one or more internal positions on at least one strand of the dsRNA agent.

In one aspect, the present invention provides a double stranded ribonucleic acid (dsRNA) agent for inhibiting expression of inhibin subunit beta E (INHBE), wherein said dsRNA agent comprises a sense strand and an antisense strand forming a double stranded region, wherein the sense strand comprises at least 15, e.g., 15, 16, 17, 18, 19, 20, or 21, contiguous nucleotides differing by no more than 0, 1, 2, 3, or 4 nucleotides from the nucleotide sequence gsascaagcaUfUfUfauacuuucuu (SEQ ID NO: 87), wherein the antisense strand comprises at least 15, e.g., 15, 16, 17, 18, 19, 20, 21, 22, or 23, contiguous nucleotides differencing by no more than 0, 1, 2, 3, or 4 nucleotides from the nucleotide sequence asdAsgadAadGuauadAaUfgcuugucsusc (SEQ ID NO:88), wherein a, g, c and u are 2′-O-methyl (2′-OMe) A, G, C, and U; Af, Gf, Cf, and Uf are 2′-fluoro A, G, C and U; dA, dG, dC and dT are 2′-deoxy A, G, C and T; s is a phosphorothioate linkage; and wherein the dsRNA comprises one or more C22 hydrocarbon chains conjugated to one or more positions on at least one strand of the dsRNA agent.

In one aspect, the present invention provides a double stranded ribonucleic acid (dsRNA) agent for inhibiting expression of inhibin subunit beta E (INHBE), wherein said dsRNA agent comprises a sense strand and an antisense strand forming a double stranded region, wherein the sense strand comprises at least 15, e.g., 15, 16, 17, 18, 19, 20, or 21, contiguous nucleotides differing by no more than 0, 1, 2, 3, or 4 nucleotides from the nucleotide sequence ascsaagcauUfUfAfuacuuucuuu (SEQ ID NO: 89), wherein the antisense strand comprises at least 15, e.g., 15, 16, 17, 18, 19, 20, 21, 22, or 23, contiguous nucleotides differencing by no more than 0, 1, 2, 3, or 4 nucleotides from the nucleotide sequence asdAsagdAadAguaudAaAfugcuuguscsu (SEQ ID NO:90), wherein a, g, c and u are 2′-O-methyl (2′-OMe) A, G, C, and U; Af, Gf, Cf, and Uf are 2′-fluoro A, G, C and U; dA, dG, dC and dT are 2′-deoxy A, G, C and T; s is a phosphorothioate linkage; and wherein the dsRNA comprises one or more C22 hydrocarbon chains conjugated to one or more internal positions on at least one strand of the dsRNA agent.

In some embodiments, the lipophilicity of the one or more C₂₂ hydrocarbon chain, measured by octanol-water partition coefficient, log K_{ow}, exceeds 0. The lipophilic moiety may possess a log K_{ow} exceeding 1, exceeding 1.5, exceeding 2, exceeding 3, exceeding 4, exceeding 5, or exceeding 10.

In some embodiments, the hydrophobicity of the dsRNA agent, measured by the unbound fraction in the plasma protein binding assay of the dsRNA agent, exceeds 0.2. In one embodiment, the plasma protein binding assay determined is an electrophoretic mobility shift assay (EMSA) using human serum albumin protein. The hydrophobicity of the dsRNA agent, measured by fraction of unbound dsRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of dsRNA/

The C₂₂ hydrocarbon chain may be saturated or unsaturated.

The C₂₂ hydrocarbon chain may be linear or branched

In some embodiments, the internal positions include all positions except the three terminal positions from each end of the at least one strand.

In some embodiments, the internal positions exclude a cleavage site region of the sense strand.

In some embodiments, the internal positions exclude positions 9-12 or positions 11-13, counting from the 5′-end of the sense strand.

In some embodiments, the internal positions exclude a cleavage site region of the antisense strand.

In some embodiments, the internal positions exclude positions 12-14, counting from the 5′ end of the antisense strand.

In some embodiments, the one or more C₂₂ hydrocarbon chains are conjugated to one or more of the following internal positions: positions 4-8 and 13-18 on the sense strand, and positions 6-10 and 15-18 on the antisense strand, counting from the 5′end of each strand.

In some embodiments, the one or more C₂₂ hydrocarbon chains are conjugated to one or more of the following internal positions: positions 5, 6, 7, 15, and 17 on the sense strand, and positions 15 and 17 on the antisense strand, counting from the 5′-end of each strand.

In some embodiments, the one or more C₂₂ hydrocarbon chains are conjugated to position 6 on the sense strand, counting from the 5′-end of the sense strand.

In some embodiments, the one or more C₂₂ hydrocarbon chains is an aliphatic, alicyclic, or polyalicyclic compound, e.g., the one or more C₂₂ hydrocarbon chains contains a functional group selected from the group consisting of hydroxyl, amine, carboxylic acid, sulfonate, phosphate, thiol, azide, and alkyne.

In some embodiments, the one or more C₂₂ hydrocarbon chains is a C22 acid, e.g. the C22 acid is selected from the group consisting of docosanoic acid, 6-octyltetradecanoic acid, 10-hexylhexadecanoic acid, all-cis-7,10,13,16,19-docosapentaenoic acid, all-cis-4,7,10,13,16,19-docosahexaenoic acid, all-cis-13,16-docosadienoic acid, all-cis-7,10,13,16-docosatetraenoic acid, all-cis-4,7,10,13,16-docosapentaenoic acid, and cis-13-docosenoic acid.

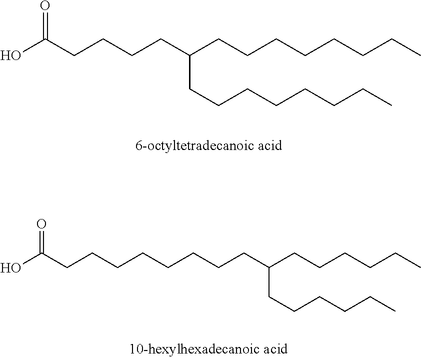 [SMILES: CCCCCCCCC(CCCCCCCC)CCCCC(=O)O ; CCCCCCC(CCCCCC)CCCCCCCCC(=O)O]

In some embodiments, the one or more C₂₂ hydrocarbon chains is a C22 alcohol, e.g., the C22 alcohol is selected from the group consisting of 1-docosanol, 6-octyltetradecan-1-ol, 10-hexylhexadecan-1-ol, cis-13-docosen-1-ol, docosan-9-ol, docosan-2-ol, docosan-10-ol, docosan-11-ol, and cis-4,7,10,13,16,19-docosahexanol.

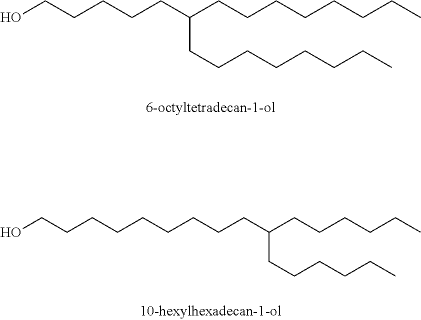 [SMILES: CCCCCCCCC(CCCCCO)CCCCCCCC ; CCCCCCC(CCCCCC)CCCCCCCCCO]

In some embodiments, the one or more C₂₂ hydrocarbon chains is a C22 amide, e.g., the C22 amide is selected from the group consisting of (E)-Docos-4-enamide, (E)-Docos-5-enamide, (Z)-Docos-9-enamide, (E)-Docos-11-enamide, 12-Docosenamide, (Z)-Docos-13-enamide, (Z)—N-Hydroxy-13-docoseneamide, (E)-Docos-14-enamide, 6-cis-Docosenamide, 14-Docosenamide Docos-11-enamide, (4E,13E)-Docosa-4,13-dienamide, and (5E,13E)-Docosa-5,13-dienamide.

The one or more C₂₂ hydrocarbon chains may be conjugated to the dsRNA agent via a direct attachment to the ribosugar of the dsRNA agent. Alternatively, the one or more C₂₂ hydrocarbon chains may be conjugated to the dsRNA agent via a linker or a carrier. In some embodiments, the one or more C₂₂ hydrocarbon chains may be conjugated to the dsRNA agent via internucleotide phosphate linkage.

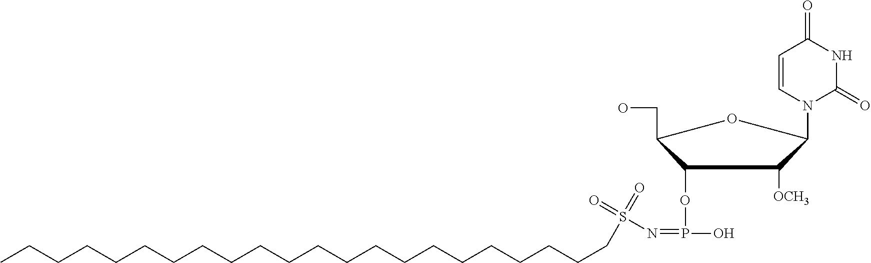

In certain embodiments, the one or more C₂₂ hydrocarbon chains is conjugated to the dsRNA agent via one or more linkers (tethers), e.g., a carrier that replaces one or more nucleotide(s) in the internal position(s).

In some embodiments, the one or more C₂₂ hydrocarbon chains is conjugated to the dsRNA agent via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In some embodiments, at least one of the linkers (tethers) is a redox cleavable linker (such as a reductively cleavable linker; e.g., a disulfide group), an acid cleavable linker (e.g., a hydrazone group, an ester group, an acetal group, or a ketal group), an esterase cleavable linker (e.g., an ester group), a phosphatase cleavable linker (e.g., a phosphate group), or a peptidase cleavable linker (e.g., a peptide bond).

In other embodiments, at least one of the linkers (tethers) is a bio-cleavable linker selected from the group consisting of DNA, RNA, disulfide, amide, functionalized monosaccharides or oligosaccharides of galactosamine, glucosamine, glucose, galactose, mannose, and combinations thereof.

In certain embodiments, the one or more C₂₂ hydrocarbon chains is conjugated to the dsRNA agent via a carrier that replaces one or more nucleotide(s). The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone or a diethanolamine backbone.

In some embodiments, the carrier replaces one or more nucleotide(s) in the internal position(s) of the dsRNA agent.

In some embodiments, the dsRNA agent further comprises a targeting ligand that targets a receptor which mediates delivery to adipose tissue. In one embodiment, the targeting ligand is selected from the group consisting of Angiopep-2, lipoprotein receptor related protein (LRP) ligand, bEnd.3 cell binding ligand, transferrin receptor (TfR) ligand, manose receptor ligand, glucose transporter protein, LDL receptor ligand, trans-retinol, RGD peptide, LDL receptor ligand, CD63 ligand, and carbohydrate based ligand.

In some embodiments, the dsRNA agent further comprises a targeting ligand that targets a liver tissue.

In one embodiment, the targeting ligand is conjugated to the 3′ end of the sense strand of the dsRNA agent.

In some embodiments, the targeting ligand is a carbohydrate-based ligand.

In one embodiment, the targeting ligand is an N-acetylgalactosamine (GalNAc) derivative.

In one embodiment, the targeting ligand is one or more GalNAc derivatives attached through a monovalent, bivalent, or trivalent branched linker.

In one embodiment, the targeting ligand is

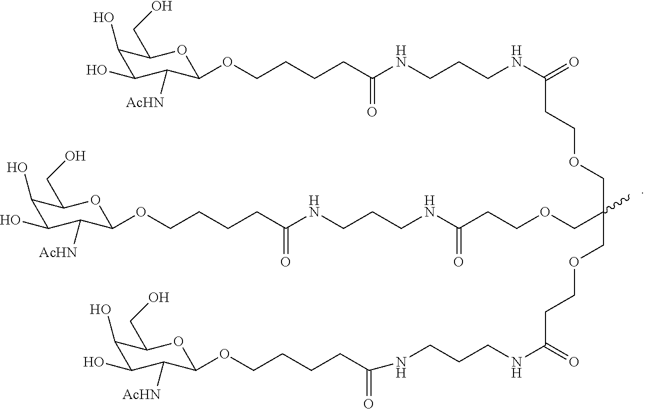 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one embodiment, the dsRNA agent is conjugated to the targeting ligand as shown in the following schematic

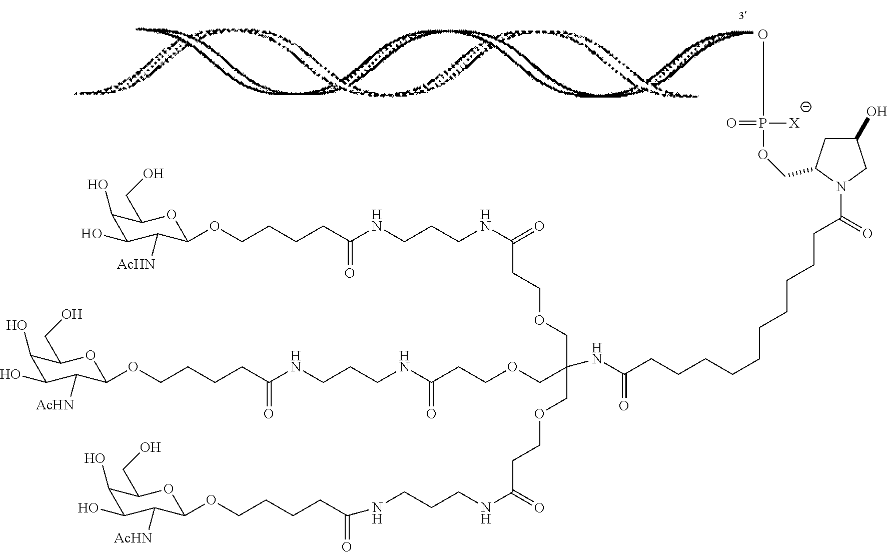

and, wherein X is O or S.

In one embodiment, the X is O.

In some embodiments, the one or more C22 hydrocarbon chains or targeting ligand is conjugated via a bio-cleavable linker selected from the group consisting of DNA, RNA, disulfide, amide, functionalized monosaccharides or oligosaccharides of galactosamine, glucosamine, glucose, galactose, mannose, and combinations thereof.

The present invention also provides cells containing any of the dsRNA agents of the invention and pharmaceutical compositions comprising any of the dsRNA agents of the invention.

The pharmaceutical composition of the invention may include dsRNA agent in an unbuffered solution, e.g., saline or water, or the pharmaceutical composition of the invention may include the dsRNA agent is in a buffer solution, e.g., a buffer solution comprising acetate, citrate, prolamine, carbonate, or phosphate or any combination thereof; or phosphate buffered saline (PBS).

In one aspect, the present invention provides a method of inhibiting expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC) in a cell, such as an adipocyte and/or a liver cell. The method includes contacting the cell with any of the dsRNAs of the invention or any of the pharmaceutical compositions of the invention, thereby inhibiting expression of the target gene in the cell.

In one embodiment, the target gene is INHBE.

In one embodiment, the target gene is ACVR1C.

In one embodiment, the target gene is PLIN1.

In one embodiment, the target gene is PDE3B.

In one embodiment, the target gene is INHBC.

In one embodiment, the cell is within a subject, e.g., a human subject, e.g., a subject having a metabolic disorder, such as diabetes, metabolic syndrome, cardiovascular disease, or hypertension.

In one embodiment, the cell is an adipocyte.

In one embodiment, the cell is a hepatocyte.

In certain embodiments, the target gene expression is inhibited by at least about 30%, 40%, 50%, 60%, 70%, 80%, 90%, or 95%. In one embodiment, inhibiting expression of the target gene decreases target gene protein level in serum of the subject by at least 30%, 40%, 50%, 60%, 70%, 80%, 90%, or 95%.

In one aspect, the present invention provides a method of treating a metabolic disorder. The method includes administering to the subject a therapeutically effective amount of any of the dsRNAs of the invention or any of the pharmaceutical compositions of the invention, thereby treating the subject having the metabolic disorder.

In another aspect, the present invention provides a method of preventing at least one symptom in a subject having a metabolic disorder. The method includes administering to the subject a prophylactically effective amount of any of the dsRNAs of the invention or any of the pharmaceutical compositions of the invention, thereby preventing at least one symptom in the subject having the metabolic disorder.

In one embodiment, the target gene is INHBE.

In one embodiment, the target gene is ACVR1C.

In one embodiment, the target gene is PLIN1.

In one embodiment, the target gene is PDE3B.

In one embodiment, the target gene is INHBC.

In one embodiment, administration of a therapeutically or prophylactically effective amount decreases the waist-to-hip ratio adjusted for body mass index in the subject.

The metabolic disorder may be, e.g. metabolic syndrome, a disorder of carbohydrates, e.g., type II diabetes, pre-diabetes, a lipid metabolism disorder, e.g., a hyperlipidemia, hypertension, lipodystrophy; a kidney disease; a cardiovascular disease, a disorder of body weight.

In some embodiments, the metabolic disorder is metabolic syndrome.

In some embodiments, the metabolic disorder is type 2 diabetes.

In some embodiments, the metabolic disorder is obesity.

In some embodiments, the metabolic disorder is elevated triglyceride level.

In some embodiments, the metabolic disorder is lipodystrophy.

In some embodiments, the metabolic disorder liver inflammation.

In some embodiments, the metabolic disorder is fatty liver disease.

In some embodiments, the metabolic disorder is hypercholesterolemia.

In some embodiments, the metabolic disorder is elevated liver enzyme.

In some embodiments, the metabolic disorder is nonalcoholic steatohepatitis (NASH).

In some embodiments, the metabolic disorder is cardiovascular disease.

In some embodiments, the metabolic disorder is hypertension.

In some embodiments, the metabolic disorder is cardiomyopathy.

In some embodiments, the metabolic disorder is heart failure.

In some embodiments, the metabolic disorder is kidney disease.

In certain embodiments, administration of the dsRNA to the subject causes a decrease target gene protein accumulation in the subject.

In a further aspect, the present invention also provides methods of inhibiting the expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC) in a subject. The methods include administering to the subject a therapeutically effective amount of any of the dsRNAs provided herein, thereby inhibiting the expression of the target gene in the subject.

In one embodiment, the subject is human.

In one embodiment, the dsRNA agent is administered to the subject at a dose of about 0.01 mg/kg to about 50 mg/kg.

In one embodiment, the dsRNA agent is administered to the subject subcutaneously.

In one embodiment, the methods of the invention include further determining the level of the target gene in a sample(s) from the subject.

In one embodiment, the level of the target gene in the subject sample(s) is a target gene protein level in a blood or serum or liver tissue sample(s).

In certain embodiments, the methods of the invention further comprise administering to the subject an additional therapeutic agent.

In certain embodiments, the additional therapeutic agent is selected from the group consisting of insulin, a glucagon-like peptide 1 agonist, a sulfonylurea, a seglitinide, a biguanide, a thiazolidinedione, an alpha-glucosidase inhibitor, an SGLT2 inhibitor, a DPP-4 inhibitor, an HMG-CoA reductase inhibitor, a statin, and a combination of any of the foregoing.

The present invention also provides kits comprising any of the dsRNAs of the invention or any of the pharmaceutical compositions of the invention, and optionally, instructions for use. In one embodiment, the invention provides a kit for performing a method of inhibiting expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC) in a cell by contacting a cell with a double stranded RNAi agent of the invention in an amount effective to inhibit expression of the target gene in the cell. The kit comprises an RNAi agent and instructions for use and, optionally, means for administering the RNAi agent to a subject.

The present invention further provides an RNA-induced silencing complex (RISC) comprising an antisense strand of any of the dsRNA agents of the invention.

### BRIEF DESCRIPTION OF THE DRAWINGS

FIG. 1 is a schematic of the study design to determine the pharmacodynamic activity of duplexes of interest targeting INHBE in non-human primates (NHP).

FIG. 2A is a graph depicting the level of INHBE mRNA in the liver of non-human primates subcutaneously administered a single 3 mg/kg dose of the indicated duplexes at Day 28 post-dose.

FIG. 2B is a graph depicting the level of INHBC mRNA in the liver of non-human primates subcutaneously administered a single 3 mg/kg dose of the indicated duplexes at Day 28 post-dose.

### DETAILED DESCRIPTION OF THE INVENTION

The present invention provides iRNA compositions which effect the RNA-induced silencing complex (RISC)-mediated cleavage of RNA transcripts of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC). The gene may be within a cell, such as an adipocyte and/or a liver cell, e.g., a cell within a subject, such as a human. The use of these iRNAs enables the targeted degradation of mRNAs of the corresponding gene (INHBE, ACVR1C, PLIN1, PDE3B, or INHBC) in mammals.

The iRNAs of the invention have been designed to target a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC), including portions of the gene that are conserved in orthologs of other mammalian species. Without intending to be limited by theory, it is believed that a combination or sub-combination of the foregoing properties and the specific target sites or the specific modifications in these iRNAs confer to the iRNAs of the invention improved efficacy, stability, potency, durability, and safety.

Accordingly, the present invention provides methods for treating and preventing a metabolic disorder, e.g. metabolic syndrome, a disorder of carbohydrates, e.g., type II diabetes, pre-diabetes, a lipid metabolism disorder, e.g., a hyperlipidemia, hypertension, lipodystrophy; a kidney disease; a cardiovascular disease, a disorder of body weight, using iRNA compositions which effect the RNA-induced silencing complex (RISC)-mediated cleavage of RNA transcripts of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC).

The iRNAs of the invention include an RNA strand (the antisense strand) having a region which is up to about 30 nucleotides or less in length, e.g., 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, which region is substantially complementary to at least part of an mRNA transcript of a metabolic disorder-associated target gene.

In certain embodiments, one or both of the strands of the double stranded RNAi agents of the invention is up to 66 nucleotides in length, e.g., 36-66, 26-36, 25-36, 31-60, 22-43, 27-53 nucleotides in length, with a region of at least 19 contiguous nucleotides that is substantially complementary to at least a part of an mRNA transcript of a metabolic disorder-associated target gene. In some embodiments, such iRNA agents having longer length antisense strands may, for example, include a second RNA strand (the sense strand) of 20-60 nucleotides in length wherein the sense and antisense strands form a duplex of 18-30 contiguous nucleotides.

The use of iRNAs of the invention enables the targeted degradation of mRNAs of the corresponding gene (INHBE, ACVR1C, PLIN1, PDE3B, or INHBC gene) in mammals. Using in vitro assays, the present inventors have demonstrated that iRNAs targeting the gene can potently mediate RNAi, resulting in significant inhibition of expression of the target gene. Thus, methods and compositions including these iRNAs are useful for treating a subject having a metabolic disorder, e.g. metabolic syndrome, a disorder of carbohydrates, e.g., type II diabetes, pre-diabetes, a lipid metabolism disorder, e.g., a hyperlipidemia, hypertension, lipodystrophy; a kidney disease; a cardiovascular disease, a disorder of body weight.

Accordingly, the present invention provides methods and combination therapies for treating a subject having a metabolic disorder that would benefit from inhibiting or reducing the expression of a metabolic disorder associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC), e.g., metabolic syndrome, a disorder of carbohydrates, e.g., type II diabetes, pre-diabetes, a lipid metabolism disorder, e.g., a hyperlipidemia, hypertension, lipodystrophy; a kidney disease; a cardiovascular disease, a disorder of body weight, using iRNA compositions which effect the RNA-induced silencing complex (RISC)-mediated cleavage of RNA transcripts of INHBE, ACVR1C, PLIN1, PDE3B, or INHBC.

The present invention also provides methods for preventing at least one symptom in a subject having a disorder that would benefit from inhibiting or reducing the expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC), e.g., metabolic syndrome, a disorder of carbohydrates, e.g., type II diabetes, pre-diabetes, a lipid metabolism disorder, e.g., a hyperlipidemia, hypertension, lipodystrophy; a kidney disease; a cardiovascular disease, a disorder of body weight.

The following detailed description discloses how to make and use compositions containing iRNAs to inhibit the expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC), as well as compositions, uses, and methods for treating subjects that would benefit from inhibition and/or reduction of the expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC), e.g., subjects susceptible to or diagnosed with a metabolic disorder.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

The term “at least”, “no less than”, or “or more” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 19 nucleotides of a 21 nucleotide nucleic acid molecule” means that 19, 20, or 21 nucleotides have the indicated property. When at least is present before a series of numbers or a range, it is understood that “at least” can modify each of the numbers in the series or range.

As used herein, “no more than” or “or less” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range. As used herein, ranges include both the upper and lower limit.

As used herein, methods of detection can include determination that the amount of analyte present is below the level of detection of the method.

In the event of a conflict between an indicated target site and the nucleotide sequence for a sense or antisense strand, the indicated sequence takes precedence.

In the event of a conflict between a sequence and its indicated site on a transcript or other sequence, the nucleotide sequence recited in the specification takes precedence.

As used herein, the term “a metabolic disorder-associated target gene” or “target gene” refers to a gene encoding “inhibin subunit beta E” (“INHBE”), “activin A receptor type 1C” (“ACVR1C”), “perilipin-1” (“PLIN1”), “phosphodiesterase 3B” (“PDE3B”), or “inhibin subunit beta C” (“INHBC”).

In one embodiment, a metabolic disorder-associated target gene is inhibin subunit beta E (INHBE).

As used herein, “inhibin subunit beta E,” used interchangeably with the terms “INHBE,” refers to a growth factor that belongs to the transforming growth factor-β(TGF-β) family. INHBE mRNA is predominantly expressed in the liver (Fang J. et al. Biochemical & Biophysical Res. Comm. 1997; 231(3):655-61), and INHBE is involved in the regulation of liver cell growth and differentiation (Chabicovsky M. et al. Endocrinology. 2003; 144(8):3497-504). INHBE is also known as inhibin beta E chain, activin E, inhibin beta E subunit, inhibin beta E, and MGC4638. More specifically, INHBE is a hepatokine which has been shown to positively correlate with insulin resistance and body mass index in humans. Quantitative real time-PCR analysis also showed an increase in INHBE gene expression in liver samples from insulin-resistant human subjects. In addition, Inhbe gene expression was shown to be increased in the livers of an art-recognized animal model of a metabolic disorder, i.e., type 2 diabetes, the db/db mouse model. Inhibition of Inhbe expression in db/db mice was demonstrated to suppress body weight gain which was attributable to diminished fat rather than lean mass.

The sequence of a human INHBE mRNA transcript can be found at, for example, GenBank Accession No. GI: 1877089956 (NM_031479.5; SEQ ID NO:1; reverse complement, SEQ ID NO: 2). The sequence of mouse INHBE mRNA can be found at, for example, GenBank Accession No. GI: 1061899809 (NM_008382.3; SEQ ID NO:3; reverse complement, SEQ ID NO:4). The sequence of rat INHBE mRNA can be found at, for example, GenBank Accession No. GI: 148747589 (NM_031815.2; SEQ ID NO:5; reverse complement, SEQ ID NO: 6). The predicted sequence of Macaca mulatta INHBE mRNA can be found at, for example, GenBank Accession No. GI: 1622845604 (XM_001115958.3; SEQ ID NO:7; reverse complement, SEQ ID NO:8).

Additional examples of INHBE mRNA sequences are readily available through publicly available databases, e.g., GenBank, UniProt, OMIM, and the Macaca genome project web site.

Further information on INHBE can be found, for example, at www.ncbi.nlm.nih.gov/gene/?term=INHBE.

The entire contents of each of the foregoing GenBank Accession numbers and the Gene database numbers are incorporated herein by reference as of the date of filing this application.

The term INHBE, as used herein, also refers to variations of the INHBE gene including variants provided in the SNP database. Numerous sequence variations within the INHBE gene have been identified and may be found at, for example, NCBI dbSNP and UniProt (see, e.g., www.ncbi.nlm.nih.gov/snp/?term=INHBE, the entire contents of which is incorporated herein by reference as of the date of filing this application.

In one embodiment, a metabolic disorder-associated target gene is activin A receptor type 1C (ACVR1C).

As used herein, “activin A receptor type 1C,” used interchangeably with the terms “ACVR1C,” refers to a type I receptor for the TGF-β family of signaling molecule. ACVR1C has intrinsic serine/threonine kinase activities in its cytoplasmic domains, inducing phosphorylation and activation of the SMAD2/3/4 complex, which translocates into the nucleus where it binds SMAD-binding elements (SBE) to activate gene transcription. Expression levels of ACVR1C varies greatly among tissues, but white and brown adipose tissues have the highest level of expression. In addition to full length protein, variants of ACVR1C are also expressed in adipose tissues, brain and ovary (Murakami M et al. Biochem Genet. 2013; 51(3-4): 202-210). ACVR1C is also known as “activin receptor-like kinase 7” (ALK-7). A polymorphism in ACVR1C has been found to be associated with increased risk of metabolic syndrome in Chinese females and may be involved in cardiovascular remodeling in patients with metabolic syndrome (Zhang, W et al. Arq Bras Gambol. 2013: 101(2):134-140). Additionally, variants predicted to lead to loss of ACVR1C gene function are thought to influence body fat distribution and protect against type 2 diabetes (Emdin C A et al. Diabetes. 2019: 68(1):226-234). A study conducted in adipocytes of an obese mouse strain demonstrated that ACVR1C dysfunction (due to a nonsense mutation) caused increased lipolysis in adipocytes and led to decreased fat accumulation, and conversely, ACVR1C activation inhibited lipolysis by suppressing the expression of adipose lipases. Furthermore, ACVR1C-deficient mice with lower body weight exhibited enhanced glucose tolerance and insulin sensitivity, and measurement of metabolic rates in these mice revealed increased O₂ consumption, decreased respiratory quotients, and increased energy expenditure (Yogosawa, S et al. Diabetes 2013: 62(1):115-123). The sequence of a human ACVR1C mRNA transcript can be found at, for example, GenBank Accession No. GI: 1519315475 (NM_145259.3, SEQ ID NO:9; reverse complement, SEQ ID NO:10), GI: 1890343165 (NM_001111031.2, SEQ ID NO:11; reverse complement, SEQ ID NO:12), GI: 1676439980 (NM_001111032.2, SEQ ID NO:13, reverse complement SEQ ID NO:14), and GI: 1676318472 (NM_001111033.2, SEQ ID NO:15, reverse complement, SEQ ID NO:16). The sequence of mouse ACVR1C mRNA can be found at, for example, GenBank Accession No. GI: 161333830 (NM_001111030.1, SEQ ID NO:17; reverse complement, SEQ ID NO:18) or GI: 161333829 (NM_001033369.3, SEQ ID NO:19; reverse complement, SEQ ID NO:20). The sequence of rat ACVR1C mRNA can be found at, for example, GenBank Accession No. GI: 1937875934 (NM_139090.2; SEQ ID NO:21; reverse complement, SEQ ID NO:22). The sequence of Macaca mulatta ACVR1C mRNA can be found at, for example, GenBank Accession No. GI: 388454445 (NM_001266690.1; SEQ ID NO:23; reverse complement, SEQ ID NO:24).

Additional examples of ACVR1C mRNA sequences are readily available through publicly available databases, e.g., GenBank, UniProt, OMIM, and the Macaca genome project web site.

Further information on ACVR1C can be found, for example, at www.ncbi.nlm.nih.gov/gene/?term=ACVR1C.

The term ACVR1C, as used herein, also refers to variations of the ACVR1C gene including variants provided in the SNP database. Numerous sequence variations within the ACVR1C gene have been identified and may be found at, for example, NCBI dbSNP and UniProt (see, e.g., www.ncbi.nlm.nih.gov/snp/?term=ACVR1C, the entire contents of which is incorporated herein by reference as of the date of filing this application).

The entire contents of each of the foregoing GenBank Accession numbers and the Gene database numbers are incorporated herein by reference as of the date of filing this application.

In one embodiment, a metabolic disorder-associated target gene is perilipin-1 (PLIN1).

As used herein, “perilipin-1,” used interchangeably with the terms “PLIN1,” refers to a protein which coats lipid storage droplets in adipocytes, thereby protecting them until they can be broken down by hormone-sensitive lipase. PLIN1 expresses predominantly in adipose tissues. PLIN1 is also known as perilipin, lipid droplet-associated protein, PERI, PLIN and FPLD4.

Constitutive overexpression of PLIN1 in cultured adipocytes has been shown to block the ability of TNF-α to increase lipolysis. In animals, separate laboratories have independently generated lines of PLIN1-null mice and observed that the mice were lean and developed systemic insulin resistance as they got older. Studies comparing lipolysis in PLIN1-null and wild-type mice revealed that PLIN1-null adipocytes had increased rates of constitutive (unstimulated) lipolysis and reduced catecholamine-stimulated lipolysis. Several studies have found that polymorphisms in the PLIN1 gene influence body weight and the risk of metabolic disease. Interestingly, one PLIN1 polymorphism was found to be associated with reduced PLIN1 expression and increased rates of basal and stimulated adipocyte lipolysis; humans with this polymorphism tend to have reduced body weight and body fat mass (Greenberg, A S et al. J Clin Invest. 2011:121(6):2102-2110). Heterozygous frameshift variants in PLIN1 have also been implicated in familial partial lipodystrophy, a rare disease characterized by a limited capacity of peripheral fat to store triglycerides, which results in metabolic abnormalities including insulin resistance, hypertriglyceridemia, abd liver steatosis (Gandotra S, Le Dour C, Bottomley W, et al. N Eng J Med 2011; 364:740-748).

The sequence of a human PLIN1 mRNA transcript can be found at, for example, GenBank Accession No. GI: 1519242647 (NM_002666.5; SEQ ID NO:25; reverse complement, SEQ ID NO:26) and GI: 1675042447 (NM_001145311.2, SEQ ID NO:27; reverse complement, SEQ ID NO:28). The sequence of mouse PLIN1 mRNA can be found at, for example, GenBank Accession No. GI: 164698407 (NM_175640.2; SEQ ID NO:29; reverse complement, SEQ ID NO:30) and GI: 164698412 (NM_001113471.1, SEQ ID NO:31; reverse complement, SEQ ID NO:32). The sequence of rat PLIN1 mRNA can be found at, for example, GenBank Accession No. GI: 815890869 (NM_001308145.1; SEQ ID NO:33; reverse complement, SEQ ID NO:34). The predicted sequence of Macaca mulatta PLIN1 mRNA can be found at, for example, GenBank Accession No. GI: 1622954660 (XM_028851317.1; SEQ ID NO:35; reverse complement, SEQ ID NO:36).

Additional examples of PLIN1 mRNA sequences are readily available through publicly available databases, e.g., GenBank, UniProt, OMIM, and the Macaca genome project web site.

Further information on PLIN1 can be found, for example, at www.ncbi.nlm.nih.gov/gene/?term=PLIN1.

The entire contents of each of the foregoing GenBank Accession numbers and the Gene database numbers are incorporated herein by reference as of the date of filing this application.

The term PLIN1, as used herein, also refers to variations of the PLIN1 gene including variants provided in the SNP database. Numerous sequence variations within the PLIN1 gene have been identified and may be found at, for example, NCBI dbSNP and UniProt (see, e.g., www.ncbi.nlm.nih.gov/snp/?term=PLIN1, the entire contents of which is incorporated herein by reference as of the date of filing this application).

In one embodiment, a metabolic disorder-associated target gene is phosphodiesterase 3B (PDE3B).

As used herein, “phosphodiesterase 3B,” used interchangeably with the terms “PDE3B,” refers to a phosphodiesterase which hydrolyzes cAMP and cGMP and is expressed in cells of importance for regulation of energy homeostasis, including adipocytes, hepatocytes, hypothalamic cells and β cells. PDE3B is also known as CGMP-inhibited 3′,5′-cyclic phosphodiesterase B, cyclic GMP-inhibited phosphodiesterase B, CGIPDE1, CGIP1 and cyclic nucleotide phosphodiesterase.

PDE3B proteins are phosphorylated and activated in hepatocytes and adipocytes in response to stimulation by insulin and/or agents that increase cAMP. Activation of PDE3B leads to increased hydrolysis of cAMP and, thereby, inhibition of catecholamine-induced lipolysis. Mice that specifically over-express PDE3B in β cells show a decrease in glucose-induced insulin secretion. PDE3B knock-out (KO) mice demonstrate a number of alterations in the regulation of energy homeostasis, including reduced fat mass, smaller adipocytes, and reduced weight gain than control mice when maintained on a high fat diet (Degerman, E. et al. CurrOpin Pharmaco. 2011:11(6):676-682).

The sequence of a human PDE3B mRNA transcript can be found at, for example, GenBank Accession No. GI: 1889438535 (NM_001363570.2; SEQ ID NO:37; reverse complement, SEQ ID NO:38), GI: 1519241942 (NM_000922.4, SEQ ID NO:39; reverse complement, SEQ ID NO:40) and GI: 1889636835 (NM_001363569.2, SEQ ID NO:41; reverse complement, SEQ ID NO:42). The sequence of mouse PDE3B mRNA can be found at, for example, GenBank Accession No. GI: 112983647 (NM_011055.2; SEQ ID NO:43; reverse complement, SEQ ID NO:44). The sequence of rat PDE3B mRNA can be found at, for example, GenBank Accession No. GI: 1939401976 (NM_017229.2; SEQ ID NO:45; reverse complement, SEQ ID NO:46). The predicted sequence of Macaca mulatta PDE3B mRNA can be found at, for example, GenBank Accession No. GI: 1622864110 (XM_015114810.2; SEQ ID NO:47; reverse complement, SEQ ID NO:48).

Additional examples of PDE3B mRNA sequences are readily available through publicly available databases, e.g., GenBank, UniProt, OMIM, and the Macaca genome project web site.

Further information on PDE3B can be found, for example, at www.ncbi.nlm.nih.gov/gene/?term=PDE3B.

The entire contents of each of the foregoing GenBank Accession numbers and the Gene database numbers are incorporated herein by reference as of the date of filing this application.

The term PDE3B, as used herein, also refers to variations of the PDE3B gene including variants provided in the SNP database. Numerous sequence variations within the PDE3B gene have been identified and may be found at, for example, NCBI dbSNP and UniProt (see, e.g., www.ncbi.nlm.nih.gov/snp/?term=PDE3B, the entire contents of which is incorporated herein by reference as of the date of filing this application.

In one embodiment, a metabolic disorder-associated target gene is inhibin subunit beta C (INHBC).

As used herein, “inhibin subunit beta C,” used interchangeably with the terms “INHBC,” refers to the beta C chain of inhibin, a member of the TGF-β superfamily. INHBC mRNA is predominantly expressed in the liver, and INHBC is involved in the regulation of liver cell growth and differentiation (Chabicovsky M. et al. Endocrinology. 2003; 144(8):3497-504). INHBC is also known as inhibin beta C chain, inhibin beta C subunit, inhibin beta C, activin C, activin beta-C chain, and IHBC. In mice, overexpression of INHBC increased total liver weight as a percentage of body weight and increased both hepatocyte proliferation and apoptosis. INHBC has been demonstrated to be significantly upregulated in obese insulin-resistant subjects (Choi, et al. Front Physiol. 2019; 10: 379). SNPs at the INHBC locus were identified as having genome-wide significance with serum urate levels, and also associated with an increase risk of gout (Yang Q, et al. 2010, Circ. Cardiovasc. Genet., 3:523-530). In addition, the INHBC locus also colocalizes with GWAS signals for estimated glomerular filtration rate (eGFR), a marker of renal function (Gudjonsson A. et al., 2022, Nature Communication, 13: 480).

The sequence of a human INHBC mRNA transcript can be found at, for example, GenBank Accession No. GI: 1519246544 (NM_005538.4; SEQ ID NO:49; reverse complement, SEQ ID NO:50). The sequence of mouse INHBC mRNA can be found at, for example, GenBank Accession No. GI: 1049480142 (NM_010565.4; SEQ ID NO:51; reverse complement, SEQ ID NO:52). The sequence of rat INHBC mRNA can be found at, for example, GenBank Accession No. GI: 59709462 (NM_022614.2; SEQ ID NO:53; reverse complement, SEQ ID NO:54). The predicted sequence of Macaca mulatta INHBC mRNA can be found at, for example, GenBank Accession No. GI: 1622845603 (XM_001115940.4; SEQ ID NO:55; reverse complement, SEQ ID NO:56).

Additional examples of INHBC mRNA sequences are readily available through publicly available databases, e.g., GenBank, UniProt, OMIM, and the Macaca genome project web site.

Further information on INHBC can be found, for example, at www.ncbi.nlm.nih.gov/gene/?term=INHBC.

The entire contents of each of the foregoing GenBank Accession numbers and the Gene database numbers are incorporated herein by reference as of the date of filing this application.

The term INHBC, as used herein, also refers to variations of the INHBC gene including variants provided in the SNP database. Numerous sequence variations within the INHBC gene have been identified and may be found at, for example, NCBI dbSNP and UniProt (see, e.g., www.ncbi.nlm.nih.gov/snp/?term=INHBC, the entire contents of which is incorporated herein by reference as of the date of filing this application).

As used herein, “target sequence” or “target nucleic acid” refers to a contiguous portion of the nucleotide sequence of an mRNA molecule formed during the transcription of a target gene, including mRNA that is a product of RNA processing of a primary transcription product. In one embodiment, the target portion of the sequence will be at least long enough to serve as a substrate for RNAi-directed cleavage at or near that portion of the nucleotide sequence of an mRNA molecule formed during the transcription of a target gene. In one embodiment, the target sequence is within the protein coding region of the target gene. In another embodiment, the target sequence is within the 3′ UTR of the target gene. The target nucleic acid can be a cellular gene (or mRNA transcribed from the gene) whose expression is associated with a particular disorder or disease state.

The target sequence may be from about 19-36 nucleotides in length, e.g., about 19-30 nucleotides in length. For example, the target sequence can be about 19-30 nucleotides, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. In certain embodiments, the target sequence is 19-23 nucleotides in length, optionally 21-23 nucleotides in length. Ranges and lengths intermediate to the above recited ranges and lengths are also contemplated to be part of the disclosure.

As used herein, the term “strand comprising a sequence” refers to an oligonucleotide comprising a chain of nucleotides that is described by the sequence referred to using the standard nucleotide nomenclature.

“G,” “C,” “A,” “T,” and “U” each generally stand for a nucleotide that contains guanine, cytosine, adenine, thymidine, and uracil as a base, respectively. However, it will be understood that the term “ribonucleotide” or “nucleotide” can also refer to a modified nucleotide, as further detailed below, or a surrogate replacement moiety (see, e.g., Table 1). The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

The terms “iRNA”, “RNAi agent,” “iRNA agent,”, “RNA interference agent” as used interchangeably herein, refer to an agent that contains RNA as that term is defined herein, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., inhibits, the expression of a INHBE, ACVR1C, PLIN1, PDE3B, or INHBC gene in a cell, e.g., a liver cell and/or adipocyte, within a subject, such as a mammalian subject.

In one embodiment, an RNAi agent of the invention includes a single stranded RNA that interacts with a target RNA sequence, e.g., a metabolic disorder-associated target mRNA sequence, to direct the cleavage of the target RNA. Without wishing to be bound by theory it is believed that long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the invention relates to a single stranded RNA (siRNA) generated within a cell and which promotes the formation of a RISC complex to effect silencing of the target gene, i.e., a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC). Accordingly, the term “siRNA” is also used herein to refer to an iRNA as described above.

In certain embodiments, the RNAi agent may be a single-stranded siRNA (ssRNAi) that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded siRNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In certain embodiments, an “iRNA” for use in the compositions, uses, and methods of the invention is a double stranded RNA and is referred to herein as a “double stranded RNA agent,” “double stranded RNA (dsRNA) molecule,” “dsRNA agent,” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA, i.e., a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC). In some embodiments of the invention, a double stranded RNA (dsRNA) triggers the degradation of a target RNA, e.g., an mRNA, through a post-transcriptional gene-silencing mechanism referred to herein as RNA interference or RNAi.

In general, the majority of nucleotides of each strand of a dsRNA molecule are ribonucleotides, but as described in detail herein, each or both strands can also include one or more non-ribonucleotides, e.g., a deoxyribonucleotide or a modified nucleotide. In addition, as used in this specification, an “iRNA” may include ribonucleotides with chemical modifications; an iRNA may include substantial modifications at multiple nucleotides. As used herein, the term “modified nucleotide” refers to a nucleotide having, independently, a modified sugar moiety, a modified internucleotide linkage, or modified nucleobase, or any combination thereof. Thus, the term modified nucleotide encompasses substitutions, additions or removal of, e.g., a functional group or atom, to internucleoside linkages, sugar moieties, or nucleobases. The modifications suitable for use in the agents of the invention include all types of modifications disclosed herein or known in the art. Any such modifications, as used in a siRNA type molecule, are encompassed by “iRNA” or “RNAi agent” for the purposes of this specification and claims.

In certain embodiments of the instant disclosure, inclusion of a deoxy-nucleotide if present within an RNAi agent can be considered to constitute a modified nucleotide.

The duplex region may be of any length that permits specific degradation of a desired target RNA through a RISC pathway, and may range from about 19 to 36 base pairs in length, e.g., about 19-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. In certain embodiments, the duplex region is 19-21 base pairs in length, e.g., 21 base pairs in length. Ranges and lengths intermediate to the above recited ranges and lengths are also contemplated to be part of the disclosure.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at least 2, 3, 4, 5, 6, 7, 8, 9, 10, 20, 23 or more unpaired nucleotides. In some embodiments, the hairpin loop can be 10 or fewer nucleotides. In some embodiments, the hairpin loop can be 8 or fewer unpaired nucleotides. In some embodiments, the hairpin loop can be 4-10 unpaired nucleotides. In some embodiments, the hairpin loop can be 4-8 nucleotides.

In certain embodiment, the two strands of double-stranded oligomeric compound can be linked together. The two strands can be linked to each other at both ends, or at one end only. By linking at one end is meant that 5′-end of first strand is linked to the 3′-end of the second strand or 3′-end of first strand is linked to 5′-end of the second strand. When the two strands are linked to each other at both ends, 5′-end of first strand is linked to 3′-end of second strand and 3′-end of first strand is linked to 5′-end of second strand. The two strands can be linked together by an oligonucleotide linker including, but not limited to, (N)n; wherein N is independently a modified or unmodified nucleotide and n is 3-23. In some embodiments, n is 3-10, e.g., 3, 4, 5, 6, 7, 8, 9, or 10. In some embodiments, the oligonucleotide linker is selected from the group consisting of GNRA, (G)4, (U)4, and (dT)4, wherein N is a modified or unmodified nucleotide and R is a modified or unmodified purine nucleotide. Some of the nucleotides in the linker can be involved in base-pair interactions with other nucleotides in the linker. The two strands can also be linked together by a non-nucleoside linker, e.g. a linker described herein. It will be appreciated by one of skill in the art that any oligonucleotide chemical modifications or variations describe herein can be used in the oligonucleotide linker.

Hairpin and dumbbell type oligomeric compounds will have a duplex region equal to or at least 14, 15, 15, 16, 17, 18, 19, 29, 21, 22, 23, 24, or 25 nucleotide pairs. The duplex region can be equal to or less than 200, 100, or 50, in length. In some embodiments, ranges for the duplex region are 15-30, 17 to 23, 19 to 23, and 19 to 21 nucleotides pairs in length.

The hairpin oligomeric compounds can have a single strand overhang or terminal unpaired region, in some embodiments at the 3′, and in some embodiments on the antisense side of the hairpin. In some embodiments, the overhangs are 1-4, more generally 2-3 nucleotides in length. The hairpin oligomeric compounds that can induce RNA interference are also referred to as “shRNA” herein.

Where the two substantially complementary strands of a dsRNA are comprised by separate RNA molecules, those molecules need not be, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs. In one embodiment of the RNAi agent, at least one strand comprises a 3′ overhang of at least 1 nucleotide. In another embodiment, at least one strand comprises a 3′ overhang of at least 2 nucleotides, e.g., 2, 3, 4, 5, 6, 7, 9, 10, 11, 12, 13, 14, or 15 nucleotides. In other embodiments, at least one strand of the RNAi agent comprises a 5′ overhang of at least 1 nucleotide. In certain embodiments, at least one strand comprises a 5′ overhang of at least 2 nucleotides, e.g., 2, 3, 4, 5, 6, 7, 9, 10, 11, 12, 13, 14, or 15 nucleotides. In still other embodiments, both the 3′ and the 5′ end of one strand of the RNAi agent comprise an overhang of at least 1 nucleotide.

In certain embodiments, an iRNA agent of the invention is a dsRNA, each strand of which comprises 19-23 nucleotides, that interacts with a target RNA sequence, e.g., a metabolic disorder-associated target gene sequence, to direct cleavage of the target RNA.

In some embodiments, an iRNA of the invention is a dsRNA of 24-30 nucleotides that interacts with a target RNA sequence, e.g., a metabolic disorder-associated target gene mRNA sequence, to direct the cleavage of the target RNA.

As used herein, the term “nucleotide overhang” refers to at least one unpaired nucleotide that protrudes from the duplex structure of a double stranded iRNA. For example, when a 3′-end of one strand of a dsRNA extends beyond the 5′-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, 3′-end, or both ends of either an antisense or sense strand of a dsRNA.

In one embodiment, the antisense strand of a dsRNA has a 1-10 nucleotide, e.g., a 1, 2, 3, 4, 5, 6, 7, 8, 9, or 10 nucleotide, overhang at the 3′-end or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the antisense strand of a dsRNA has a 1-10 nucleotide, e.g., 0-3, 1-3, 2-4, 2-5, 4-10, 5-10, e.g., a 1, 2, 3, 4, 5, 6, 7, 8, 9, or 10 nucleotide, overhang at the 3′-end or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the antisense strand of a dsRNA has a 1-10 nucleotides, e.g., a 1, 2, 3, 4, 5, 6, 7, 8, 9, or 10 nucleotide, overhang at the 3′-end or the 5′-end. In certain embodiments, the overhang on the sense strand or the antisense strand, or both, can include extended lengths longer than 10 nucleotides, e.g., 1-30 nucleotides, 2-30 nucleotides, 10-30 nucleotides, 10-25 nucleotides, 10-20 nucleotides, or 10-15 nucleotides in length. In certain embodiments, an extended overhang is on the sense strand of the duplex. In certain embodiments, an extended overhang is present on the 3′ end of the sense strand of the duplex. In certain embodiments, an extended overhang is present on the 5′ end of the sense strand of the duplex. In certain embodiments, an extended overhang is on the antisense strand of the duplex. In certain embodiments, an extended overhang is present on the 3′end of the antisense strand of the duplex. In certain embodiments, an extended overhang is present on the 5′end of the antisense strand of the duplex. In certain embodiments, one or more of the nucleotides in the extended overhang is replaced with a nucleoside thiophosphate. In certain embodiments, the overhang includes a self-complementary portion such that the overhang is capable of forming a hairpin structure that is stable under physiological conditions.

“Blunt” or “blunt end” means that there are no unpaired nucleotides at that end of the double stranded RNA agent, i.e., no nucleotide overhang. A “blunt ended” double stranded RNA agent is double stranded over its entire length, i.e., no nucleotide overhang at either end of the molecule. The RNAi agents of the invention include RNAi agents with no nucleotide overhang at one end (i.e., agents with one overhang and one blunt end) or with no nucleotide overhangs at either end. Most often such a molecule will be double-stranded over its entire length.

The term “antisense strand” or “guide strand” refers to the strand of an iRNA, e.g., a dsRNA, which includes a region that is substantially complementary to a target sequence, e.g., a metabolic disorder-associated target gene mRNA.

As used herein, the term “region of complementarity” refers to the region on the antisense strand that is substantially complementary to a sequence, for example a target sequence, e.g., an INHBE nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, or 3 nucleotides of the 5′- or 3′-end of the iRNA. In some embodiments, a double stranded RNA agent of the invention includes a nucleotide mismatch in the antisense strand. In some embodiments, the antisense strand of the double stranded RNA agent of the invention includes no more than 4 mismatches with the target mRNA, e.g., the antisense strand includes 4, 3, 2, 1, or 0 mismatches with the target mRNA. In some embodiments, the antisense strand double stranded RNA agent of the invention includes no more than 4 mismatches with the sense strand, e.g., the antisense strand includes 4, 3, 2, 1, or 0 mismatches with the sense strand. In some embodiments, a double stranded RNA agent of the invention includes a nucleotide mismatch in the sense strand. In some embodiments, the sense strand of the double stranded RNA agent of the invention includes no more than 4 mismatches with the antisense strand, e.g., the sense strand includes 4, 3, 2, 1, or 0 mismatches with the antisense strand. In some embodiments, the nucleotide mismatch is, for example, within 5, 4, 3 nucleotides from the 3′-end of the iRNA. In another embodiment, the nucleotide mismatch is, for example, in the 3′-terminal nucleotide of the iRNA agent. In some embodiments, the mismatch(s) is not in the seed region.

Thus, an RNAi agent as described herein can contain one or more mismatches to the target sequence. In one embodiment, an RNAi agent as described herein contains no more than 3 mismatches (i.e., 3, 2, 1, or 0 mismatches). In one embodiment, an RNAi agent as described herein contains no more than 2 mismatches. In one embodiment, an RNAi agent as described herein contains no more than 1 mismatch. In one embodiment, an RNAi agent as described herein contains 0 mismatches. In certain embodiments, if the antisense strand of the RNAi agent contains mismatches to the target sequence, the mismatch can optionally be restricted to be within the last 5 nucleotides from either the 5′- or 3′-end of the region of complementarity. For example, in such embodiments, for a 23 nucleotide RNAi agent, the strand which is complementary to a region of a metabolic disorder-associated target gene, generally does not contain any mismatch within the central 13 nucleotides. The methods described herein or methods known in the art can be used to determine whether an RNAi agent containing a mismatch to a target sequence is effective in inhibiting the expression of a target gene. Consideration of the efficacy of RNAi agents with mismatches in inhibiting expression of an INHBE, ACVR1C, PLIN1, PDE3B, or INHBC target gene is important, especially if the particular region of complementarity in the target gene is known to have polymorphic sequence variation within the population.

The term “sense strand” or “passenger strand” as used herein, refers to the strand of an iRNA that includes a region that is substantially complementary to a region of the antisense strand as that term is defined herein.

As used herein, “substantially all of the nucleotides are modified” are largely but not wholly modified and can include not more than 5, 4, 3, 2, or 1 unmodified nucleotides.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3, or 2 mismatched base pairs upon hybridization for a duplex up to 30 base pairs, while retaining the ability to hybridize under the conditions most relevant to their ultimate application, e.g., inhibition of gene expression, in vitro or in vivo. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogsteen base pairing.

The terms “complementary,” “fully complementary” and “substantially complementary” herein can be used with respect to the base matching between the sense strand and the antisense strand of a dsRNA, or between two oligonucleotides or polynucleotides, such as the antisense strand of a double stranded RNA agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding a metabolic disorder-associated target gene). For example, a polynucleotide is complementary to at least a part of a metabolic disorder-associated target gene mRNA if the sequence is substantially complementary to a non-interrupted portion of an mRNA encoding a metabolic disorder-associated target gene.

Accordingly, in some embodiments, the antisense strand polynucleotides disclosed herein are fully complementary to the target gene sequence.

In some embodiments, the antisense strand polynucleotides disclosed herein are substantially complementary to the target gene sequence and comprise a contiguous nucleotide sequence which is at least about 80% complementary over its entire length to the equivalent region of the nucleotide sequence of SEQ ID NOs: 1, 3, 5, or 7 for INHBE, or a fragment of SEQ ID NOs: 1, 3, 5, or 7, such as about 85%, 86%, 87%, 88%, 89%, 90%, 91%, 92%, 93%, 94%, 95%, 96%, 97%, 98%, or about 99% complementary.

In some embodiments, the antisense polynucleotides disclosed herein are substantially complementary to a fragment of a target INHBE sequence and comprise a contiguous nucleotide sequence which is at least 80% complementary over its entire length to a fragment of SEQ ID NO: 1 selected from the group of nucleotides 400-422, 410-432, 518-540, 519-541, 640-662, 1430-1452, 1863-1885, or 1864-1886 of SEQ ID NO: 1, such as about 85%, about 90%, about 91%, about 92%, about 93%, about 94%, about 95%, about 96%, about 97%, about 98%, or about 99% complementary.

In other embodiments, the antisense polynucleotides disclosed herein are substantially complementary to the target INHBE sequence and comprise a contiguous nucleotide sequence which is at least about 80% complementary over its entire length to any one of the sense strand nucleotide sequences in any one of Tables 2-3, or a fragment of any one of the sense strand nucleotide sequences in any one of Tables 2-3, such as about 85%, 86%, 87%, 88%, 89%, 90%, 91%, 92%, 93%, 94%, 95%, 96%, 97%, 98%, or about 99% complementary.

In one embodiment, an RNAi agent of the disclosure includes a sense strand that is substantially complementary to an antisense polynucleotide which, in turn, is the same as a target INHBE sequence, and wherein the sense strand polynucleotide comprises a contiguous nucleotide sequence which is at least about 80% complementary over its entire length to the equivalent region of the nucleotide sequence of SEQ ID NOs: 2, 4, 6, or 8, or a fragment of any one of SEQ ID NOs: 2, 4, 6, or 8, such as about 86%, 87%, 88%, 89%, 90%, 91%, 92%, 93%, 94%, 95%, 96%, 97%, 98%, or about 99% complementary.

In some embodiments, an iRNA of the invention includes a sense strand that is substantially complementary to an antisense polynucleotide which, in turn, is complementary to a target INHBE sequence, and wherein the sense strand polynucleotide comprises a contiguous nucleotide sequence which is at least about 80% complementary over its entire length to any one of the antisense strand nucleotide sequences in any one of any one of Tables 2-3, or a fragment of any one of the antisense strand nucleotide sequences in any one of Tables 2-3, such as about 86%, 87%, 88%, 89%, 90%, 91%, 92%, 93%, 94%, 95%, 96%, 97%, 98%, or about 99% complementary.

In some embodiments, the sense and antisense strands are selected from any one of duplexes AD-1706583, AD-1711744, AD-1706593, AD-1708473, AD-1706662, AD-1706761, AD-1707306, AD-1707639, and AD-1707640.

In some embodiments, the sense and antisense strands are selected from duplex AD-1706583.

In some embodiments, the sense and antisense strands are selected from duplex AD-1711744.

In some embodiments, the sense and antisense strands are selected from duplex AD-1706593.

In some embodiments, the sense and antisense strands are selected from duplex AD-1708473.

In some embodiments, the sense and antisense strands are selected from duplex AD-1706662.

In some embodiments, the sense and antisense strands are selected from duplex AD-1706761.

In some embodiments, the sense and antisense strands are selected from duplex AD-1707306.

In some embodiments, the sense and antisense strands are selected from duplex AD-1707639.

In some embodiments, the sense and antisense strands are selected from duplex AD-1707640.

In other embodiments, the antisense strand polynucleotides disclosed herein are substantially complementary to the target gene sequence and comprise a contiguous nucleotide sequence which is at least about 80% complementary over its entire length to the equivalent region of the nucleotide sequence of SEQ ID NOs: 9, 11, 13, 15, 17, 19, 21, or 23 for ACVR1C, or a fragment of SEQ ID NOs: 9, 11, 13, 15, 17, 19, 21, or 23, such as about 85%, 86%, 87%, 88%, 89%, 90%, 91%, 92%, 93%, 94%, 95%, 96%, 97%, 98%, or about 99% complementary.

In other embodiments, the antisense polynucleotides disclosed herein are substantially complementary to the target ACVR1C sequence and comprise a contiguous nucleotide sequence which is at least about 80% complementary over its entire length to any one of the sense strand nucleotide sequences in any one of Tables 4-7, or a fragment of any one of the sense strand nucleotide sequences in any one of Tables 4-7, such as about 85%, 86%, 87%, 88%, 89%, 90%, 91%, 92%, 93%, 94%, 95%, 96%, 97%, 98%, or about 99% complementary.

In one embodiment, an RNAi agent of the disclosure includes a sense strand that is substantially complementary to an antisense polynucleotide which, in turn, is the same as a target ACVR1C sequence, and wherein the sense strand polynucleotide comprises a contiguous nucleotide sequence which is at least about 80% complementary over its entire length to the equivalent region of the nucleotide sequence of SEQ ID NOs: 10, 12, 14, 16, 18, 20, 22, or 24, or a fragment of any one of SEQ ID NOs: 10, 12, 14, 16, 18, 20, 22, or 24, such as about 86%, 87%, 88%, 89%, 90%, 91%, 92%, 93%, 94%, 95%, 96%, 97%, 98%, or about 99% complementary.

In some embodiments, an iRNA of the invention includes a sense strand that is substantially complementary to an antisense polynucleotide which, in turn, is complementary to a target ACVR1C sequence, and wherein the sense strand polynucleotide comprises a contiguous nucleotide sequence which is at least about 80% complementary over its entire length to any one of the antisense strand nucleotide sequences in any one of any one of Tables 4-7, or a fragment of any one of the antisense strand nucleotide sequences in any one of Tables 4-7, such as about 86%, 87%, 88%, 89%, 90%, 91%, 92%, 93%, 94%, 95%, 96%, 97%, 98%, or about 99% complementary.

In other embodiments, the antisense strand polynucleotides disclosed herein are substantially complementary to the target gene sequence and comprise a contiguous nucleotide sequence which is at least about 80% complementary over its entire length to the equivalent region of the nucleotide sequence of SEQ ID NOs:25, 27, 29, 31, 33, or 35 for PLIN1, or a fragment of SEQ ID NOs: 25, 27, 29, 31, 33, or 35, such as about 85%, 86%, 87%, 88%, 89%, 90%, 91%, 92%, 93%, 94%, 95%, 96%, 97%, 98%, or about 99% complementary.

In other embodiments, the antisense polynucleotides disclosed herein are substantially complementary to the target PLIN1 sequence and comprise a contiguous nucleotide sequence which is at least about 80% complementary over its entire length to any one of the sense strand nucleotide sequences in any one of Tables 8-11, or a fragment of any one of the sense strand nucleotide sequences in any one of Tables 8-11, such as about 85%, 86%, 87%, 88%, 89%, 90%, 91%, 92%, 93%, 94%, 95%, 96%, 97%, 98%, or about 99% complementary.

In one embodiment, an RNAi agent of the disclosure includes a sense strand that is substantially complementary to an antisense polynucleotide which, in turn, is the same as a target PLIN1 sequence, and wherein the sense strand polynucleotide comprises a contiguous nucleotide sequence which is at least about 80% complementary over its entire length to the equivalent region of the nucleotide sequence of SEQ ID NOs: 26, 28, 30, 32, 34, or 36, or a fragment of any one of SEQ ID NOs: 26, 28, 30, 32, 34, or 36, such as about 86%, 87%, 88%, 89%, 90%, 91%, 92%, 93%, 94%, 95%, 96%, 97%, 98%, or about 99% complementary.

In some embodiments, an iRNA of the invention includes a sense strand that is substantially complementary to an antisense polynucleotide which, in turn, is complementary to a target PLIN1 sequence, and wherein the sense strand polynucleotide comprises a contiguous nucleotide sequence which is at least about 80% complementary over its entire length to any one of the antisense strand nucleotide sequences in any one of any one of Tables 8-11, or a fragment of any one of the antisense strand nucleotide sequences in any one of Tables 8-11, such as about 86%, 87%, 88%, 89%, 90%, 91%, 92%, 93%, 94%, 95%, 96%, 97%, 98%, or about 99% complementary.

In other embodiments, the antisense strand polynucleotides disclosed herein are substantially complementary to the target gene sequence and comprise a contiguous nucleotide sequence which is at least about 80% complementary over its entire length to the equivalent region of the nucleotide sequence of SEQ ID NOs:37, 39, 41, 43, 45, or 47 for PDE3B, or a fragment of SEQ ID NOs: 37, 39, 41, 43, 45, or 47, such as about 85%, 86%, 87%, 88%, 89%, 90%, 91%, 92%, 93%, 94%, 95%, 96%, 97%, 98%, or about 99% complementary.

In other embodiments, the antisense polynucleotides disclosed herein are substantially complementary to the target PDE3B sequence and comprise a contiguous nucleotide sequence which is at least about 80% complementary over its entire length to any one of the sense strand nucleotide sequences in any one of Tables 12-15, or a fragment of any one of the sense strand nucleotide sequences in any one of Tables 12-15, such as about 85%, 86%, 87%, 88%, 89%, 90%, 91%, 92%, 93%, 94%, 95%, 96%, 97%, 98%, or about 99% complementary.

In one embodiment, an RNAi agent of the disclosure includes a sense strand that is substantially complementary to an antisense polynucleotide which, in turn, is the same as a target PDE3B sequence, and wherein the sense strand polynucleotide comprises a contiguous nucleotide sequence which is at least about 80% complementary over its entire length to the equivalent region of the nucleotide sequence of SEQ ID NOs: 38, 40, 42, 44, 46, or 48, or a fragment of any one of SEQ ID NOs: 38, 40, 42, 44, 46, or 48, such as about 86%, 87%, 88%, 89%, 90%, 91%, 92%, 93%, 94%, 95%, 96%, 97%, 98%, or about 99% complementary.

In some embodiments, an iRNA of the invention includes a sense strand that is substantially complementary to an antisense polynucleotide which, in turn, is complementary to a target PDE3B sequence, and wherein the sense strand polynucleotide comprises a contiguous nucleotide sequence which is at least about 80% complementary over its entire length to any one of the antisense strand nucleotide sequences in any one of any one of Tables 12-15, or a fragment of any one of the antisense strand nucleotide sequences in any one of Tables 12-15, such as about 86%, 87%, 88%, 89%, 90%, 91%, 92%, 93%, 94%, 95%, 96%, 97%, 98%, or about 99% complementary.

In other embodiments, the antisense strand polynucleotides disclosed herein are substantially complementary to the target gene sequence and comprise a contiguous nucleotide sequence which is at least about 80% complementary over its entire length to the equivalent region of the nucleotide sequence of SEQ ID NOs:49, 51, 53, or 55 for INHBC, or a fragment of SEQ ID NOs: 49, 51, 53, or 55, such as about 85%, 86%, 87%, 88%, 89%, 90%, 91%, 92%, 93%, 94%, 95%, 96%, 97%, 98%, or about 99% complementary.

In other embodiments, the antisense polynucleotides disclosed herein are substantially complementary to the target INHBC sequence and comprise a contiguous nucleotide sequence which is at least about 80% complementary over its entire length to any one of the sense strand nucleotide sequences in any one of Tables 16-17, or a fragment of any one of the sense strand nucleotide sequences in any one of Tables 16-17, such as about 85%, 86%, 87%, 88%, 89%, 90%, 91%, 92%, 93%, 94%, 95%, 96%, 97%, 98%, or about 99% complementary.

In one embodiment, an RNAi agent of the disclosure includes a sense strand that is substantially complementary to an antisense polynucleotide which, in turn, is the same as a target INHBC sequence, and wherein the sense strand polynucleotide comprises a contiguous nucleotide sequence which is at least about 80% complementary over its entire length to the equivalent region of the nucleotide sequence of SEQ ID NOs: 50, 52, 54, or 56, or a fragment of any one of SEQ ID NOs: 50, 52, 54, or 56, such as about 86%, 87%, 88%, 89%, 90%, 91%, 92%, 93%, 94%, 95%, 96%, 97%, 98%, or about 99% complementary.

In some embodiments, an iRNA of the invention includes a sense strand that is substantially complementary to an antisense polynucleotide which, in turn, is complementary to a target INHBC sequence, and wherein the sense strand polynucleotide comprises a contiguous nucleotide sequence which is at least about 80% complementary over its entire length to any one of the antisense strand nucleotide sequences in any one of any one of Tables 16-17, or a fragment of any one of the antisense strand nucleotide sequences in any one of Tables 16-17, such as about 86%, 87%, 88%, 89%, 90%, 91%, 92%, 93%, 94%, 95%, 96%, 97%, 98%, or about 99% complementary.

In some embodiments, the double-stranded region of a double-stranded iRNA agent is equal to or at least, 10, 11, 12, 13, 14, 15, 16, 17, 18, 19, 20, 21, 22, 23, 23, 24, 25, 26, 27, 28, 29, 30 or more nucleotide pairs in length.

In some embodiments, the antisense strand of a double-stranded iRNA agent is equal to or at least 14, 15, 16, 17, 18, 19, 20, 21, 22, 23, 23, 24, 25, 26, 27, 28, 29, or 30 nucleotides in length.

In some embodiments, the sense strand of a double-stranded iRNA agent is equal to or at least 10, 11, 12, 13, 14, 15, 16, 17, 18, 19, 20, 21, 22, 23, 23, 24, 25, 26, 27, 28, 29, or 30 nucleotides in length.

In one embodiment, the sense and antisense strands of the double-stranded iRNA agent are each independently 15 to 30 nucleotides in length.

In one embodiment, the sense and antisense strands of the double-stranded iRNA agent are each independently 19 to 25 nucleotides in length.

In one embodiment, the sense and antisense strands of the double-stranded iRNA agent are each independently 21 to 23 nucleotides in length.

In one embodiment, the sense strand of the iRNA agent is 21-nucleotides in length, and the antisense strand is 23-nucleotides in length, wherein the strands form a double-stranded region of 21 consecutive base pairs having a 2-nucleotide long single stranded overhangs at the 3′-end.

In general, an “iRNA” includes ribonucleotides with chemical modifications. Such modifications may include all types of modifications disclosed herein or known in the art. Any such modifications, as used in a dsRNA molecule, are encompassed by “iRNA” for the purposes of this specification and claims.

In certain embodiments of the instant disclosure, inclusion of a deoxy-nucleotide if present within an RNAi agent can be considered to constitute a modified nucleotide.

In an aspect of the invention, an agent for use in the methods and compositions of the invention is a single-stranded antisense oligonucleotide molecule that inhibits a target mRNA via an antisense inhibition mechanism. The single-stranded antisense oligonucleotide molecule is complementary to a sequence within the target mRNA. The single-stranded antisense oligonucleotides can inhibit translation in a stoichiometric manner by base pairing to the mRNA and physically obstructing the translation machinery, see Dias, N. et al., (2002) Mol Cancer Ther 1:347-355. The single-stranded antisense oligonucleotide molecule may be about 14 to about 30 nucleotides in length and have a sequence that is complementary to a target sequence. For example, the single-stranded antisense oligonucleotide molecule may comprise a sequence that is at least about 14, 15, 16, 17, 18, 19, 20, or more contiguous nucleotides from any one of the antisense sequences described herein.

The phrase “contacting a cell with an iRNA,” such as a dsRNA, as used herein, includes contacting a cell by any possible means. Contacting a cell with an iRNA includes contacting a cell in vitro with the iRNA or contacting a cell in vivo with the iRNA. The contacting may be done directly or indirectly. Thus, for example, the iRNA may be put into physical contact with the cell by the individual performing the method, or alternatively, the iRNA may be put into a situation that will permit or cause it to subsequently come into contact with the cell.

Contacting a cell in vitro may be done, for example, by incubating the cell with the iRNA. Contacting a cell in vivo may be done, for example, by injecting the iRNA into or near the tissue where the cell is located, or by injecting the iRNA into another area, e.g., the bloodstream or the subcutaneous space, such that the agent will subsequently reach the tissue where the cell to be contacted is located. For example, the iRNA may contain or be coupled to a targeting ligand, e.g., GalNAc, that directs the iRNA to a site of interest, e.g., the liver. In other embodiments, the RNAi agent may contain or be coupled to one or more C22 hydrocarbon chains and one or more GalNAc derivatives. In other embodiments, the RNAi agent contains or is coupled to one or more C22 hydrocarbon chains and does not contain or is not coupled to one or more GalNAc derivatives. Combinations of in vitro and in vivo methods of contacting are also possible. For example, a cell may also be contacted in vitro with an RNAi agent and subsequently transplanted into a subject.

In certain embodiments, contacting a cell with an iRNA includes “introducing” or “delivering the iRNA into the cell” by facilitating or effecting uptake or absorption into the cell. Absorption or uptake of an iRNA can occur through unaided diffusion or active cellular processes, or by auxiliary agents or devices. Introducing an iRNA into a cell may be in vitro or in vivo. For example, for in vivo introduction, iRNA can be injected into a tissue site or administered systemically. In vitro introduction into a cell includes methods known in the art such as electroporation and lipofection. Further approaches are described herein below or are known in the art.

The term “lipid nanoparticle” or “LNP” is a vesicle comprising a lipid layer encapsulating a pharmaceutically active molecule, such as a nucleic acid molecule, e.g., an iRNA or a plasmid from which an iRNA is transcribed. LNPs are described in, for example, U.S. Pat. Nos. 6,858,225, 6,815,432, 8,158,601, and 8,058,069, the entire contents of which are hereby incorporated herein by reference.

As used herein, a “subject” is an animal, such as a mammal, including a primate (such as a human, a non-human primate, e.g., a monkey, and a chimpanzee), a non-primate (such as a cow, a pig, a horse, a goat, a rabbit, a sheep, a hamster, a guinea pig, a cat, a dog, a rat, or a mouse), or a bird that expresses the target gene, either endogenously or heterologously. In an embodiment, the subject is a human, such as a human being treated or assessed for a disease or disorder that would benefit from reduction in metabolic disorder-associated target gene expression; a human at risk for a disease or disorder that would benefit from reduction in metabolic disorder-associated target gene expression; a human having a disease or disorder that would benefit from reduction in metabolic disorder-associated target gene expression; or human being treated for a disease or disorder that would benefit from reduction in metabolic disorder-associated target gene expression as described herein. In some embodiments, the subject is a female human. In other embodiments, the subject is a male human. In one embodiment, the subject is an adult subject. In another embodiment, the subject is a pediatric subject.

As used herein, the terms “treating” or “treatment” refer to a beneficial or desired result, such as reducing at least one sign or symptom of a metabolic disorder in a subject. Treatment also includes a reduction of one or more sign or symptoms associated with unwanted metabolic disorder-associated target gene expression; diminishing the extent of unwanted metabolic disorder-associated target gene activation or stabilization; amelioration or palliation of unwanted metabolic disorder-associated target gene activation or stabilization. “Treatment” can also mean prolonging survival as compared to expected survival in the absence of treatment.

The term “lower” in the context of the level a metabolic disorder-associated target gene in a subject or a disease marker or symptom refers to a statistically significant decrease in such level. The decrease can be, for example, at least 10%, 15%, 20%, 25%, 30%, %, 40%, 45%, 50%, 55%, 60%, 65%, 70%, 75%, 80%, 85%, 90%, 95%, or more. In certain embodiments, a decrease is at least 20%. In certain embodiments, the decrease is at least 50% in a disease marker, e.g., protein or gene expression level. “Lower” in the context of the level of a metabolic disorder-associated target gene in a subject is a decrease to a level accepted as within the range of normal for an individual without such disorder. In certain embodiments, “lower” is the decrease in the difference between the level of a marker or symptom for a subject suffering from a disease and a level accepted within the range of normal for an individual. The term “lower” can also be used in association with normalizing a symptom of a disease or condition, i.e. decreasing the difference between a level in a subject suffering from a metabolic disorder towards or to a level in a normal subject not suffering from an metabolic disorder. As used herein, if a disease is associated with an elevated value for a symptom, “normal” is considered to be the upper limit of normal. If a disease is associated with a decreased value for a symptom, “normal” is considered to be the lower limit of normal.

As used herein, “prevention” or “preventing,” when used in reference to a disease, disorder or condition thereof, may be treated or ameliorated by a reduction in expression of a metabolic disorder-associated target gene, refers to a reduction in the likelihood that a subject will develop a symptom associated with such a disease, disorder, or condition, e.g., a symptom of a metabolic disorder, e.g., diabetes. The failure to develop a disease, disorder or condition, or the reduction in the development of a symptom associated with such a disease, disorder or condition (e.g., by at least about 10% on a clinically accepted scale for that disease or disorder), or the exhibition of delayed symptoms delayed (e.g., by days, weeks, months or years) is considered effective prevention.

The treatment and prophylactic methods of the invention are useful for treating any disease or disorder that is caused by, or associated with INHBE, ACVR1C, PLIN1, PDE3B, and/or INHBC gene expression or INHBE, ACVR1C, PLIN1, PDE3B, and/or INHBC protein production and includes a disease, disorder or condition that would benefit from a decrease in INHBE, ACVR1C, PLIN1, PDE3B, and/or INHBC gene expression, replication, or protein activity such as a metabolic disorder. In some embodiments, the metabolic disorder is metabolic syndrome.

A “metabolic disorder” is a disorder that disrupts normal metabolism, the process of converting food to energy on a cellular level. Metabolic diseases affect the ability of the cell to perform critical biochemical reactions that involve the processing or transport of proteins (amino acids), carbohydrates (sugars and starches), or lipids (fatty acids).

For example, metabolic disorders may be associated with a body fat distribution characterized by higher accumulation of fat around the waist (such as greater abdominal fat or larger waist circumference) and/or lower accumulation of fat around the hips (such as lower gluteofemoral fat or smaller hip circumference), resulting in a greater waist-to-hip ratio (WHR), and higher cardio-metabolic risk independent of body mass index (BMI).

Non-limiting examples of metabolic diseases include disorders of carbohydrates, e.g., diabetes, type I diabetes, type II diabetes, galactosemia, hereditary fructose intolerance, fructose 1,6-diphosphatase deficiency, glycogen storage disorders, congenital disorders of glycosylation, insulin resistance, insulin insufficiency, hyperinsulinemia, impaired glucose tolerance (IGT), abnormal glycogen metabolism; disorders of amino acid metabolism, e.g., maple syrup urine disease (MSUD), or homocystinuria; disorder of organic acid metabolism, e.g., methylmalonic aciduria, 3-methylglutaconic aciduria-Barth syndrome, glutaric aciduria or 2-hydroxyglutaric aciduria—D and L forms; disorders of fatty acid beta-oxidation, e.g., medium-chain acyl-CoA dehydrogenase deficiency (MCAD), long-chain 3-hydroxyacyl-CoA dehydrogenase deficiency (LCHAD), very-long-chain acyl-CoA dehydrogenase deficiency (VLCAD); disorders of lipid metabolism, e.g., GM1 Gangliosidosis, Tay-Sachs Disease, Sandhoff Disease, Fabry Disease, Gaucher Disease, Niemann-Pick Disease, Krabbe Disease, Mucolipidoses, or Mucopolysaccharidoses; disorders of lipid distribution and/or storage, e.g., lipodystrophy, mitochondrial disorders, e.g., mitochondrial cardiomyopathies; Leigh disease; mitochondrial encephalopathy, lactic acidosis, and stroke-like episodes (MELAS); myoclonic epilepsy with ragged-red fibers (MERRF); neuropathy, ataxia, and retinitis pigmentosa (NARP); Barth syndrome; peroxisomal disorders, e.g., Zellweger Syndrome (cerebrohepatorenal syndrome), X-Linked Adrenoleukodystrophy or Refsum Disease.

In certain embodiment, metabolic disorders are associated with body fat distribution and include, but are not limited to metabolic syndrome, type 2 diabetes, hyperlipidemia or dyslipidemia (high or altered circulating levels of low-density lipoprotein cholesterol (LDL-C), triglycerides, very low-density lipoprotein cholesterol (VLDL-C), apolipoprotein B or other lipid fractions), obesity (particularly abdominal obesity), lipodystrophy (such as an inability to deposit fat in adipose depots regionally (partial lipodystrophy) or in the whole body (lipoatrophy)), insulin resistance or higher or altered insulin levels at fasting or during a metabolic challenge, liver fat deposition or fatty liver disease and their complications (such as, for example, cirrhosis, fibrosis, or inflammation of the liver), nonalcoholic steatohepatitis, other types of liver inflammation, higher or elevated or altered liver enzyme levels or other markers of liver damage, inflammation or fat deposition in the liver, higher blood pressure and/or hypertension, higher blood sugar or glucose or hyperglycemia, metabolic syndrome, coronary artery disease, and other atherosclerotic conditions, and the complications of each of the aforementioned conditions.

In one embodiment, a metabolic disorder is metabolic syndrome. The term “metabolic syndrome,” as used herein, is disorder that includes a clustering of components that reflect overnutrition, sedentary lifestyles, genetic factors, increasing age, and resultant excess adiposity. Metabolic syndrome includes the clustering of abdominal obesity, insulin resistance, dyslipidemia, and elevated blood pressure and is associated with other comorbidities including the prothrombotic state, proinflammatory state, nonalcoholic fatty liver disease, and reproductive disorders. The prevalence of the metabolic syndrome has increased to epidemic proportions not only in the United States and the remainder of the urbanized world but also in developing nations. Metabolic syndrome is associated with an approximate doubling of cardiovascular disease risk and a 5-fold increased risk for incident type 2 diabetes mellitus.

Abdominal adiposity (e.g., a large waist circumference (high waist-to-hip ratio)), high blood pressure, insulin resistance and dislipidemia are central to metabolic syndrome and its individual components (e.g., central obesity, fasting blood glucose (FBG)/pre-diabetes/diabetes, hypercholesterolemia, hypertriglyceridemia, and hypertension).

In one embodiment, a metabolic disorder is a disorder of carbohydrates. In one embodiment, the disorder of carbohydrates is diabetes.

As used herein, the term “diabetes” refers to a group of metabolic disorders characterized by high blood sugar (glucose) levels which result from defects in insulin secretion or action, or both. There are two most common types of diabetes, namely type 1 diabetes and type 2 diabetes, which both result from the body's inability to regulate insulin. Insulin is a hormone released by the pancreas in response to increased levels of blood sugar (glucose) in the blood.

The term “type I diabetes,” as used herein, refers to a chronic disease that occurs when the pancreas produces too little insulin to regulate blood sugar levels appropriately. Type I diabetes is also referred to as insulin-dependent diabetes mellitus, IDDM, and juvenile onset diabetes. People with type I diabetes (insulin-dependent diabetes) produce little or no insulin at all. Although about 6 percent of the United States population has some form of diabetes, only about 10 percent of all diabetics have type I disorder. Most people who have type I diabetes developed the disorder before age 30. Type 1 diabetes represents the result of a progressive autoimmune destruction of the pancreatic β-cells with subsequent insulin deficiency. More than 90 percent of the insulin-producing cells (beta cells) of the pancreas are permanently destroyed. The resulting insulin deficiency is severe, and to survive, a person with type I diabetes must regularly inject insulin.

In type II diabetes (also referred to as noninsulin-dependent diabetes mellitus, NDDM), the pancreas continues to manufacture insulin, sometimes even at higher than normal levels. However, the body develops resistance to its effects, resulting in a relative insulin deficiency. Type II diabetes may occur in children and adolescents but usually begins after age 30 and becomes progressively more common with age: about 15 percent of people over age 70 have type II diabetes. Obesity is a risk factor for type II diabetes, and 80 to 90 percent of the people with this disorder are obese.

In some embodiments, diabetes includes pre-diabetes. “Pre-diabetes” refers to one or more early diabetic conditions including impaired glucose utilization, abnormal or impaired fasting glucose levels, impaired glucose tolerance, impaired insulin sensitivity and insulin resistance. Prediabetes is a major risk factor for the development of type 2 diabetes mellitus, cardiovascular disease and mortality. Much focus has been given to developing therapeutic interventions that prevent the development of type 2 diabetes by effectively treating prediabetes.

Diabetes can be diagnosed by the administration of a glucose tolerance test. Clinically, diabetes is often divided into several basic categories. Primary examples of these categories include, autoimmune diabetes mellitus, non-insulin-dependent diabetes mellitus (type 1 NDDM), insulin-dependent diabetes mellitus (type 2 IDDM), non-autoimmune diabetes mellitus, non-insulin-dependent diabetes mellitus (type 2 NIDDM), and maturity-onset diabetes of the young (MODY). A further category, often referred to as secondary, refers to diabetes brought about by some identifiable condition which causes or allows a diabetic syndrome to develop. Examples of secondary categories include, diabetes caused by pancreatic disease, hormonal abnormalities, drug- or chemical-induced diabetes, diabetes caused by insulin receptor abnormalities, diabetes associated with genetic syndromes, and diabetes of other causes. (see e.g., Harrison's (1996) 14th ed., New York, McGraw-Hill).

In one embodiment, a metabolic disorder is a lipid metabolism disorder. As used herein, a “lipid metabolism disorder” or “disorder of lipid metabolism” refers to any disorder associated with or caused by a disturbance in lipid metabolism. This term also includes any disorder, disease or condition that can lead to hyperlipidemia, or condition characterized by abnormal elevation of levels of any or all lipids and/or lipoproteins in the blood. This term refers to an inherited disorder, such as familial hypertriglyceridemia, familial partial lipodystrophy type 1 (FPLD1), or an induced or acquired disorder, such as a disorder induced or acquired as a result of a disease, disorder or condition (e.g., renal failure), a diet, or intake of certain drugs (e.g., as a result of highly active antiretroviral therapy (HAART) used for treating, e.g., AIDS or HIV). This term also refers to a disorder of fat distribution/storage, e.g., lipodystrophy.

Additional examples of disorders of lipid metabolism include, but are not limited to, atherosclerosis, dyslipidemia, hypertriglyceridemia (including drug-induced hypertriglyceridemia, diuretic-induced hypertriglyceridemia, alcohol-induced hypertriglyceridemia, β-adrenergic blocking agent-induced hypertriglyceridemia, estrogen-induced hypertriglyceridemia, glucocorticoid-induced hypertriglyceridemia, retinoid-induced hypertriglyceridemia, cimetidine-induced hypertriglyceridemia, and familial hypertriglyceridemia), acute pancreatitis associated with hypertriglyceridemia, chylomicron syndrome, familial chylomicronemia, Apo-E deficiency or resistance, LPL deficiency or hypoactivity, hyperlipidemia (including familial combined hyperlipidemia), hypercholesterolemia, lipodystrophy, gout associated with hypercholesterolemia, xanthomatosis (subcutaneous cholesterol deposits), hyperlipidemia with heterogeneous LPL deficiency, hyperlipidemia with high LDL and heterogeneous LPL deficiency, fatty liver disease, or non-alcoholic stetohepatitis (NASH).

Cardiovascular diseases are also considered “metabolic disorders”, as defined herein. These diseases may include coronary artery disease (also called ischemic heart disease), hypertension, inflammation associated with coronary artery disease, restenosis, peripheral vascular diseases, and stroke.

Kidney diseases are also considered “bolic disorders”, as defined herein. Such diseases may include chronic kidney disease, diabetic nephrophathy, diabetic kidney disease, or gout.

Disorders related to body weight are also considered “metabolic disorders”, as defined herein. Such disorders may include obesity, hypo-metabolic states, hypothyroidism, uremia, and other conditions associated with weight gain (including rapid weight gain), weight loss, maintenance of weight loss, or risk of weight regain following weight loss.

Blood sugar disorders are further considered “metabolic disorders”, as defined herein. Such disorders may include diabetes, hypertension, and polycystic ovarian syndrome related to insulin resistance. Other exemplary disorders of metabolic disorders may also include renal transplantation, nephrotic syndrome, Cushing's syndrome, acromegaly, systemic lupus erythematosus, dysglobulinemia, lipodystrophy, glycogenosis type I, and Addison's disease.

In one embodiment, a metabolic disorder is primary hypertension. “Primary hypertension” is a result of environmental or genetic causes (e.g., a result of no obvious underlying medical cause).

In one embodiment, a metabolic disorder is secondary hypertension. “Secondary hypertension” has an identifiable underlying disorder which can be of multiple etiologies, including renal, vascular, and endocrine causes, e.g., renal parenchymal disease (e.g., polycystic kidneys, glomerular or interstitial disease), renal vascular disease (e.g., renal artery stenosis, fibromuscular dysplasia), endocrine disorders (e.g., adrenocorticosteroid or mineralocorticoid excess, pheochromocytoma, hyperthyroidism or hypothyroidism, growth hormone excess, hyperparathyroidism), coarctation of the aorta, or oral contraceptive use.

In one embodiment, a metabolic disorder is resistant hypertension. “Resistant hypertension” is blood pressure that remains above goal (e.g., above 130 mm Hg systolic or above 90 diastolic) in spite of concurrent use of three antihypertensive agents of different classes, one of which is a thiazide diuretic. Subjects whose blood pressure is controlled with four or more medications are also considered to have resistant hypertension.

Additional diseases or conditions related to metabolic disorders that would be apparent to the skilled artisan and are within the scope of this disclosure.

“Therapeutically effective amount,” as used herein, is intended to include the amount of an RNAi agent that, when administered to a subject having a metabolic disorder, is sufficient to effect treatment of the disease (e.g., by diminishing, ameliorating, or maintaining the existing disease or one or more symptoms of disease). The “therapeutically effective amount” may vary depending on the RNAi agent, how the agent is administered, the disease and its severity and the history, age, weight, family history, genetic makeup, the types of preceding or concomitant treatments, if any, and other individual characteristics of the subject to be treated.

“Prophylactically effective amount,” as used herein, is intended to include the amount of an RNAi agent that, when administered to a subject having a metabolic disorder, is sufficient to prevent or ameliorate the disease or one or more symptoms of the disease. Ameliorating the disease includes slowing the course of the disease or reducing the severity of later-developing disease. The “prophylactically effective amount” may vary depending on the RNAi agent, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of an RNAi agent that produces some desired effect at a reasonable benefit/risk ratio applicable to any treatment. The iRNA employed in the methods of the present invention may be administered in a sufficient amount to produce a reasonable benefit/risk ratio applicable to such treatment.

The phrase “pharmaceutically acceptable” is employed herein to refer to those compounds, materials, compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Such carriers are known in the art. Pharmaceutically acceptable carriers include carriers for administration by injection.

The term “sample,” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs, or localized regions. For example, samples may be derived from particular organs, parts of organs, or fluids or cells within those organs. In certain embodiments, samples may be derived from the liver (e.g., whole liver or certain segments of liver or certain types of cells in the liver, such as, e.g., hepatocytes). In some embodiments, a “sample derived from a subject” refers to urine obtained from the subject. A “sample derived from a subject” can refer to blood or blood derived serum or plasma from the subject.

### II. iRNAs of the Invention

The present invention provides iRNAs which inhibit the expression of a metabolic disorder-associated target gene, e.g., INHBE, ACVR1C, PLIN1, PDE3B, or INHBC. In certain embodiments, the iRNA includes double stranded ribonucleic acid (dsRNA) molecules for inhibiting the expression of a metabolic disorder-associated target gene in a cell (e.g., an adipocyte and/or a hepatocyte), such as a cell within a subject, e.g., a mammal, such as a human susceptible to developing a metabolic disorder, e.g., metabolic syndrome, a disorder of carbohydrates, e.g., type II diabetes, pre-diabetes, a lipid metabolism disorder, e.g., a hyperlipidemia, hypertension, lipodystrophy; a kidney disease; a cardiovascular disease, a disorder of body weight. The dsRNAi agent includes an antisense strand having a region of complementarity which is complementary to at least a part of an mRNA formed in the expression of a metabolic disorder-associated target gene. The region of complementarity is about 19-30 nucleotides in length (e.g., about 30, 29, 28, 27, 26, 25, 24, 23, 22, 21, 20, or 19 nucleotides in length).

Upon contact with a cell expressing the target gene, the iRNA inhibits the expression of the target gene (e.g., a human, a primate, a non-primate, or a rat INHBE, ACVR1C, PLIN1, PDE3B, or INHBC gene) by at least about 50% as assayed by, for example, a PCR or branched DNA (bDNA)-based method, or by a protein-based method, such as by immunofluorescence analysis, using, for example, western blotting or flow cytometric techniques. In certain embodiments, inhibition of expression is determined by the qPCR method provided in the examples herein with the siRNA at, e.g., a 10 nM concentration, in an appropriate organism cell line provided therein. In certain embodiments, inhibition of expression in vivo is determined by knockdown of the human gene in a rodent expressing the human gene, e.g., a mouse or an AAV-infected mouse expressing the human target gene, e.g., when administered as single dose, e.g., at 3 mg/kg at the nadir of RNA expression.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of an INHBE, ACVR1C, PLIN1, PDE3B, or INHBC gene. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. In certain embodiments, the duplex structure is 18 to 25 base pairs in length, e.g., 18-25, 18-24, 18-23, 18-22, 18-21, 18-20, 19-25, 19-24, 19-23, 19-22, 19-21, 19-20, 20-25, 20-24, 20-23, 20-22, 20-21, 21-25, 21-24, 21-23, 21-22, 22-25, 22-24, 22-23, 23-25, 23-24 or 24-25 base pairs in length, for example, 19-21 basepairs in length. Ranges and lengths intermediate to the above recited ranges and lengths are also contemplated to be part of the disclosure.

Similarly, the region of complementarity to the target sequence is 15 to 30 nucleotides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, for example 19-23 nucleotides in length or 21-23 nucleotides in length. Ranges and lengths intermediate to the above recited ranges and lengths are also contemplated to be part of the disclosure.

In some embodiments, the duplex structure is 19 to 30 base pairs in length. Similarly, the region of complementarity to the target sequence is 19 to 30 nucleotides in length.

In some embodiments, the dsRNA is about 19 to about 23 nucleotides in length, or about 25 to about 30 nucleotides in length. In general, the dsRNA is long enough to serve as a substrate for the Dicer enzyme. For example, it is well-known in the art that dsRNAs longer than about 21-23 nucleotides in length may serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 19 to about 30 base pairs, e.g., about 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, an iRNA agent useful to target INHBE, ACVR1C, PLIN1, PDE3B, or INHBC gene expression is not generated in the target cell by cleavage of a larger dsRNA.

A dsRNA as described herein can further include one or more single-stranded nucleotide overhangs, e.g., 1-4, 2-4, 1-3, 2-3, 1, 2, 3, or 4 nucleotides. dsRNAs having at least one nucleotide overhang can have superior inhibitory properties relative to their blunt-ended counterparts. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, 3′-end, or both ends of an antisense or sense strand of a dsRNA.

A dsRNA can be synthesized by standard methods known in the art. Double stranded RNAi compounds of the invention may be prepared using a two-step procedure. First, the individual strands of the double stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Similarly, single-stranded oligonucleotides of the invention can be prepared using solution-phase or solid-phase organic synthesis or both.

In an aspect, a dsRNA of the invention includes at least two nucleotide sequences, a sense sequence and an anti-sense sequence. The sense strand is selected from the group of sequences provided in any one of Tables 2-17, 19 and 20, and the corresponding antisense strand of the sense strand is selected from the group of sequences of any one of Tables 2-17, 19 and 20. In this aspect, one of the two sequences is complementary to the other of the two sequences, with one of the sequences being substantially complementary to a sequence of an mRNA generated in the expression of a-associated target gene. As such, in this aspect, a dsRNA will include two oligonucleotides, where one oligonucleotide is described as the sense strand in any one of Tables 2-17, 19 and 20, and the second oligonucleotide is described as the corresponding antisense strand of the sense strand in any one of Tables 2-17, 19 and 20.

In certain embodiments, the substantially complementary sequences of the dsRNA are contained on separate oligonucleotides. In other embodiments, the substantially complementary sequences of the dsRNA are contained on a single oligonucleotide.

In some embodiments, the sense or antisense strands are selected from the sense or antisense strand of any one of duplexes AD-1706583, AD-1711744, AD-1706593, AD-1708473, AD-1706662, AD-1706761, AD-1707306, AD-1707639, and AD-1707640.

In some embodiments, the sense or antisense strands are selected from the sense or antisense strand of duplex AD-1706583.

In some embodiments, the sense or antisense strands are selected from the sense or antisense strand of duplex AD-1711744.

In some embodiments, the sense or antisense strands are selected from the sense or antisense strand of duplex AD-1706593.

In some embodiments, the sense or antisense strands are selected from the sense or antisense strand of duplex AD-1708473.

In some embodiments, the sense or antisense strands are selected from the sense or antisense strand of duplex AD-1706662.

In some embodiments, the sense or antisense strands are selected from the sense or antisense strand of duplex AD-1706761.

In some embodiments, the sense or antisense strands are selected from the sense or antisense strand of duplex AD-1707306.

In some embodiments, the sense or antisense strands are selected from the sense or antisense strand of duplex AD-1707639.

In some embodiments, the sense or antisense strands are selected from the sense or antisense strand of duplex AD-1707640.

It will be understood that, although the sequences in, for example, Table 2, are not described as modified or conjugated sequences, the RNA of the iRNA of the invention e.g., a dsRNA of the invention, may comprise any one of the sequences set forth in any one of Tables 2-17, 19 and 20 that is un-modified, un-conjugated, or modified or conjugated differently than described therein. In other words, the invention encompasses dsRNA of Tables 2-17, 19 and 20 which are un-modified, un-conjugated, modified, or conjugated, as described herein.

The skilled person is well aware that dsRNAs having a duplex structure of about 20 to 23 base pairs, e.g., 21, base pairs have been hailed as particularly effective in inducing RNA interference (Elbashir et al., EMBO 2001, 20:6877-6888). However, others have found that shorter or longer RNA duplex structures can also be effective (Chu and Rana (2007) RNA 14:1714-1719; Kim et al. (2005) Nat Biotech 23:222-226). In the embodiments described above, by virtue of the nature of the oligonucleotide sequences provided in any one of Tables 2-17, 19 and 20. dsRNAs described herein can include at least one strand of a length of minimally 21 nucleotides. It can be reasonably expected that shorter duplexes having any one of the sequences in any one of Tables 2-17, 19 and 20 minus only a few nucleotides on one or both ends can be similarly effective as compared to the dsRNAs described above. Hence, dsRNAs having a sequence of at least 19, 20, or more contiguous nucleotides derived from any one of the sequences of any one of Tables 2-17, 19 and 20, and differing in their ability to inhibit the expression of an INHBE gene by not more than about 5, 10, 15, 20, 25, or 30% inhibition from a dsRNA comprising the full sequence, are contemplated to be within the scope of the present invention.

In addition, the RNAs provided in Tables 2-17, 19 and 20 identify a site(s) in a metabolic disorder-associated target gene transcript that is susceptible to RISC-mediated cleavage. As such, the present invention further features iRNAs that target within one of these sites. As used herein, an iRNA is said to target within a particular site of an RNA transcript if the iRNA promotes cleavage of the transcript anywhere within that particular site. Such an iRNA will generally include at least about 19 contiguous nucleotides from any one of the sequences provided in any one of Tables 2-17, 19 and 20 coupled to additional nucleotide sequences taken from the region contiguous to the selected sequence in a metabolic disorder-associated target gene.

### III. Modifications for the RNAi Agents of the Invention

In certain embodiments, the RNA of the iRNA of the invention e.g., a dsRNA, is un modified, and does not comprise, e.g., chemical modifications or conjugations known in the art and described herein. In other embodiments, the RNA of an iRNA of the invention, e.g., a dsRNA, is chemically modified to enhance stability or other beneficial characteristics. In certain embodiments of the invention, substantially all of the nucleotides of an iRNA of the invention are modified. In other embodiments of the invention, all of the nucleotides of an iRNA or substantially all of the nucleotides of an iRNA are modified, i.e., not more than 5, 4, 3, 2, or 1 unmodified nucleotides are present in a strand of the iRNA.

In some embodiments, the dsRNA agents of the invention comprise at least one nucleic acid modification described herein. For example, at least one modification selected from the group consisting of modified internucleoside linkage, modified nucleobase, modified sugar, and any combinations thereof. Without limitations, such a modification can be present anywhere in the dsRNA agent of the invention. For example, the modification can be present in one of the RNA molecules.

In one embodiment, the dsRNA agents of the disclosure comprise one or more C22 hydrocarbon chains conjugated to one or more internal positions on at least one strand and do not comprise additional chemical modifications known in the art and described herein, in the remaining positions of the sense and anti-sense strands.

In some embodiments, the dsRNA agents of the invention comprise one or more C22 hydrocarbon chains conjugated to one or more internal positions on at least one strand, and comprise at least one additional nucleic acid modification described herein. For example, at least one modification selected from the group consisting of modified internucleoside linkage, modified nucleobase, modified sugar, and any combinations thereof. Without limitations, such a modification can be present anywhere in the dsRNA agent of the invention. For example, the modification can be present in one of the RNA molecules.

In one embodiment, the dsRNA agents of the disclosure comprise one or more targeting ligands, e.g., one or more GalNAc derivatives, and do not comprise additional chemical modifications known in the art and described herein, in the remaining positions of the sense and anti-sense strands.

In some embodiments, the dsRNA agents of the invention comprise one or more targeting ligands, e.g., one or more GalNAc derivatives, and comprise at least one additional nucleic acid modification described herein. For example, at least one modification selected from the group consisting of modified internucleoside linkage, modified nucleobase, modified sugar, and any combinations thereof. Without limitations, such a modification can be present anywhere in the dsRNA agent of the invention. For example, the modification can be present in one of the RNA molecules.

Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. Specific examples of RNAi agents useful in the embodiments described herein include, but are not limited to, RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments, a modified RNAi agent will have a phosphorus atom in its internucleoside backbone.

### A. Nucleobase Modifications

The naturally occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleoside backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. The unmodified or natural nucleobases can be modified or replaced to provide iRNAs having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the oligomer modifications described herein. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

An oligomeric compound described herein can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Exemplary modified nucleobases include, but are not limited to, other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8 (amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N4-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil, 4-(thio)pseudouracil, 2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5 (alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5 (methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6 (azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any nucleobase that can base pair with all of the four naturally occurring nucleobases without substantially affecting the melting behavior, recognition by intracellular enzymes or activity of the iRNA duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P. Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic include more complicated structures, such as for example a tricyclic phenoxazine nucleobase mimetic. Methods for preparation of the above noted modified nucleobases are well known to those skilled in the art.

### B. Sugar Modifications

DsRNA agent of the inventions provided herein can comprise one or more (e.g., 1, 2, 3, 4, 5, 6, 7, 8, 9, 10, 11, 12, 13, 14, 15 or more) monomer, including a nucleoside or nucleotide, having a modified sugar moiety. For example, the furanosyl sugar ring of a nucleoside can be modified in a number of ways including, but not limited to, addition of a substituent group, bridging of two non-geminal ring atoms to form a locked nucleic acid or bicyclic nucleic acid. In certain embodiments, oligomeric compounds comprise one or more (e.g., 1, 2, 3, 4, 5, 6, 7, 8, 9, 10, 11, 12, 13, 14, 15 or more) monomers that are LNA.

In some embodiments of a locked nucleic acid, the 2′ position of furnaosyl is connected to the 4′ position by a linker selected independently from —[C(R1)(R2)]ₙ—, —[C(R1)(R2)]ₙ—O—, [C(R1)(R2)]ₙ—N(R1)-, —[C(R1)(R2)]ₙ—N(R1)-O—, —[C(R1R2)]ₙ—O—N(R1)-, —C(R1)=(R2)-O—, —C(R1)=N—, —C(R1)=N—O—, —C(═NR1)-, —C(═NR1)-O—, —C(═O)—, —C(═O)O—, —C(═S)—, —C(═S)O—, —C(═S)S—, —O—, —Si(R1)2-, —S(═O)ₓ— and —N(R1)-;
wherein:
x is 0, 1, or 2;
n is 1, 2, 3, or 4;
each R1 and R2 is, independently, H, a protecting group, hydroxyl, C1-C12 alkyl, substituted C1-C12 alkyl, C2-C12 alkenyl, substituted C2-C12 alkenyl, C2-C12 alkynyl, substituted C2-C12 alkynyl, C5-C20 aryl, substituted C5-C20 aryl, heterocycle radical, substituted heterocycle radical, heteroaryl, substituted heteroaryl, C5-C7 alicyclic radical, substituted C5-C7 alicyclic radical, halogen, OJ1, NJ1J2, SJ1, N3, COOJ1, acyl (C(═O)—H), substituted acyl, CN, sulfonyl (S(═O)2-J1), or sulfoxyl (S(═O)-J1); and
each J1 and J2 is, independently, H, C1-C12 alkyl, substituted C1-C12 alkyl, C2-C12 alkenyl,
substituted C2-C12 alkenyl, C2-C12 alkynyl, substituted C2-C12 alkynyl, C5-C20 aryl, substituted C5-C20 aryl, acyl (C(═O)—H), substituted acyl, a heterocycle radical, a substituted heterocycle radical, C1-C12 aminoalkyl, substituted C1-C12 aminoalkyl or a protecting group.

In some embodiments, each of the linkers of the LNA compounds is, independently, —[C(R1)(R2)]n-, —[C(R1)(R2)]n-O—, —C(R1R2) N(R1)-O— or —C(R1R2)-O—N(R1)-. In another embodiment, each of said linkers is, independently, 4′-CH₂-2′, 4′-(CH₂)₂-2′, 4′-(CH₂)₃-2′, 4′—CH₂—O-2′, 4′-(CH₂)₂—O-2′, 4′-CH₂—O—N(R1)-2′ and 4′-CH₂—N(R1)-O-2′- wherein each R1 is, independently, H, a protecting group or C1-C12 alkyl.

Certain LNA's have been prepared and disclosed in the patent literature as well as in scientific literature (Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; WO 94/14226; WO 2005/021570; Singh et al., J. Org. Chem., 1998, 63, 10035-10039; Examples of issued US patents and published applications that disclose LNA s include, for example, U.S. Pat. Nos. 7,053,207; 6,268,490; 6,770,748; 6,794,499; 7,034,133; and 6,525,191; and U.S. Pre-Grant Publication Nos. 2004-0171570; 2004-0219565; 2004-0014959; 2003-0207841; 2004-0143114; and 20030082807.

Also provided herein are LNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., Cuff. Opinion Invens. Drugs, 2001, 2, 558-561; Braasch et al., Chem. Biol., 2001, 8 1-7; and Orum et al., CWT. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O-2′) LNA is used for the bicyclic moiety; in the case of an ethylene group in this position, the term ethyleneoxy (4′-CH₂CH₂—O-2′) LNA is used (Singh et al., Chem. Commun., 1998, 4, 455-456: Morita et al., Bioorganic Medicinal Chemistry, 2003, 11, 2211-2226). Methyleneoxy (4′-CH₂—O-2′) LNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., Proc. Natl. Acad. Sci. U.S.A., 2000, 97, 5633-5638).

An isomer of methyleneoxy (4′-CH₂—O-2′) LNA that has also been discussed is alpha-L methyleneoxy (4′-CH₂—O-2′) LNA which has been shown to have superior stability against a 3′-exonuclease. The alpha-L-methyleneoxy (4′-CH₂—O-2′) LNA's were incorporated into antisense gapmers and chimeras that showed potent antisense activity (Frieden et al., Nucleic Acids Research, 2003, 21, 6365-6372).

The synthesis and preparation of the methyleneoxy (4′-CH₂—O-2′) LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) LNA, phosphorothioate-methyleneoxy (4′-CH₂—O-2′) LNA and 2′-thio-LNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described Mengel et al., WO 99/14226). Furthermore, synthesis of 2′-amino-LNA, a novel comformationally restricted high-affinity oligonucleotide analog has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

Modified sugar moieties are well known and can be used to alter, typically increase, the affinity of the antisense compound for its target and/or increase nuclease resistance. A representative list of preferred modified sugars includes but is not limited to bicyclic modified sugars, including methyleneoxy (4′-CH₂—O-2′) LNA and ethyleneoxy (4′-(CH₂)₂—O-2′ bridge) ENA; substituted sugars, especially 2′-substituted sugars having a 2′-F, 2′-OCH₃ or a 2′-O(CH₂)₂—OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR, n=1-50; “locked” nucleic acids (LNA) in which the furanose portion of the nucleoside includes a bridge connecting two carbon atoms on the furanose ring, thereby forming a bicyclic ring system; O-AMINE or O—(CH₂)ₙAMINE (n=1-10, AMINE=NH₂; alkylamino, dialkylamino, heterocyclyl, arylamino, diaryl amino, heteroaryl amino, diheteroaryl amino, ethylene diamine or polyamino); and O—CH₂CH₂(NCH₂CH₂NMe₂)₂.

“Deoxy” modifications include hydrogen (i.e. deoxyribose sugars, which are of particular relevance to the single-strand overhangs); halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino); NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

Other suitable 2′-modifications, e.g., modified MOE, are described in U.S. Patent Application Publication No. 20130130378, contents of which are herein incorporated by reference.

A modification at the 2′ position can be present in the arabinose configuration The term “arabinose configuration” refers to the placement of a substituent on the C2′ of ribose in the same configuration as the 2′-OH is in the arabinose.

The sugar can comprise two different modifications at the same carbon in the sugar, e.g., gem modification. The sugar group can also contain one or more carbons that possess the opposite stereochemical configuration than that of the corresponding carbon in ribose. Thus, an oligomeric compound can include one or more monomers containing e.g., arabinose, as the sugar. The monomer can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

DsRNA agent of the inventions disclosed herein can also include abasic sugars, i.e., a sugar which lack a nucleobase at C-1′ or has other chemical groups in place of a nucleobase at C1′. See for example U.S. Pat. No. 5,998,203, content of which is herein incorporated in its entirety. These abasic sugars can also be further containing modifications at one or more of the constituent sugar atoms. DsRNA agent of the inventions can also contain one or more sugars that are the L isomer, e.g. L-nucleosides. Modification to the sugar group can also include replacement of the 4′-0 with a sulfur, optionally substituted nitrogen or CH₂ group. In some embodiments, linkage between C1′ and nucleobase is in a configuration.

Sugar modifications can also include acyclic nucleotides, wherein a C—C bonds between ribose carbons (e.g., C1′-C2′, C2′-C3′, C3′-C4′, C4′-O4′, C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

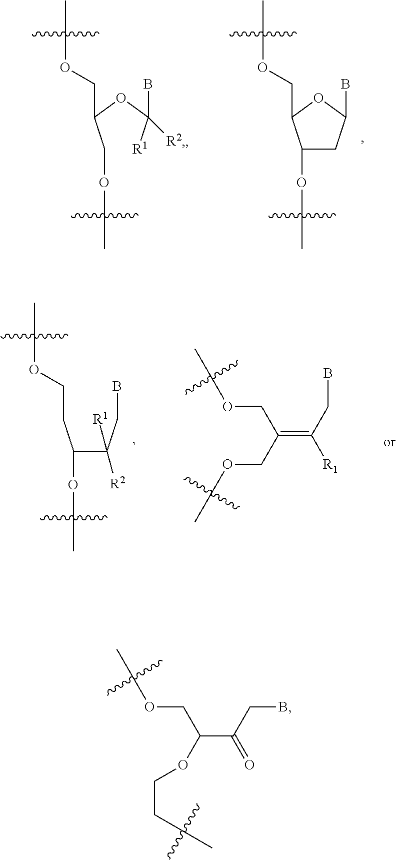

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

In some embodiments, sugar modifications are selected from the group consisting of 2′-H, 2′-O-Me (2′-O-methyl), 2′-O-MOE (2′-O-methoxyethyl), 2′-F, 2′-O-[2-(methylamino)-2-oxoethyl] (2′-O-NMA), 2′-S methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE) and gem 2′-OMe/2′F with 2′-O-Me in the arabinose configuration.

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of O, S, S(O), SO₂, N(R′), C(O), N(R′)C(O)O, OC(O)N(R′), CH(Z′), phosphorous containing linkage, optionally substituted aryl, optionally substituted heteroaryl, optionally substituted heterocyclic or optionally substituted cycloalkyl, where R′ is hydrogen, acyl or optionally substituted aliphatic, Z′ is selected from the group consisting of OR₁₁, COR₁₁, CO₂R₁₁,

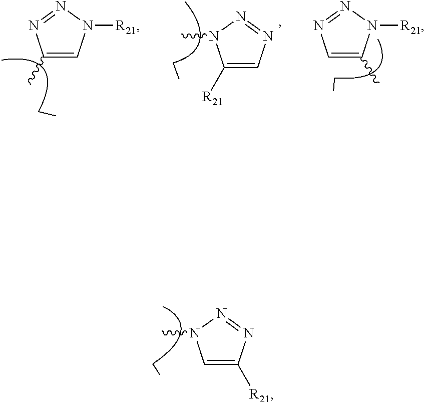

NR₂₁R₃₁, CONR₂₁R₃₁, CON(H)NR₂₁R₃₁, ONR₂₁R₃₁, CON(H)N═R₄₁R₅₁, N(R₂₁)C(═NR₃₁)NR₂₁R₃₁, N(R₂₁)C(O)NR₂₁R₃₁, N(R₂₁)C(S)NR₂₁R₃₁, OC(O)NR₂₁R₃₁, SC(O)NR₂₁R₃₁, N(R₂₁)C(S)OR₁₁, N(R₂₁)C(O)OR₁₁, N(R₂₁)C(O)SR₁₁, N(R₂₁)N═R₄₁R₅₁, ON═C₄₁R₅₁, SO₂R₁₁, SOR₁₁, SR₁₁, and substituted or unsubstituted heterocyclic; R₂₁ and R₃₁ for each occurrence are independently hydrogen, acyl, unsubstituted or substituted aliphatic, aryl, heteroaryl, heterocyclic, OR₁₁, COR₁₁, CO₂R₁₁, or NR₁₁R₁₁′; or R₂₁ and R₃₁, taken together with the atoms to which they are attached, form a heterocyclic ring; R₄₁ and R₅₁ for each occurrence are independently hydrogen, acyl, unsubstituted or substituted aliphatic, aryl, heteroaryl, heterocyclic, OR₁₁, COR₁₁, or CO₂R₁₁, or NR₁₁R₁₁′; and R₁₁ and R₁₁′ are independently hydrogen, aliphatic, substituted aliphatic, aryl, heteroaryl, or heterocyclic. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alki metal or transition metal with an overall charge of +1; and Y is O, S, or NR′, where R′ is hydrogen, optionally substituted aliphatic. Preferably this modification is at the 5 terminal of the iRNA.

In certain embodiments, LNA's include bicycle nucleotide having the formula:

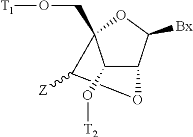

wherein:
   Bx is a heterocyclic base moiety;
   T₁ is H or a hydroxyl protecting group;
   T₂ is H, a hydroxyl protecting group or a reactive phosphorus group;
   Z is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In some embodiments, each of the substituted groups, is, independently, mono or poly substituted with optionally protected substituent groups independently selected from halogen, oxo, hydroxyl, OJ1, NJ1J2, SJ1, N3, OC(═X)J1, OC(═X)NJ1J2, NJ3C(═X)NJ1J2 and CN, wherein each J1, J2 and J3 is, independently, H or C₁-C₆ alkyl, and X is O, S or NJ1.

In certain such embodiments, each of the substituted groups, is, independently, mono or poly substituted with substituent groups independently selected from halogen, oxo, hydroxyl, OJ1, NJ1J2, SJ1, N3, OC(═X)J1, and NJ3C(═X)NJ1J2, wherein each J1, J2 and J3 is, independently, H, C₁-C₆ alkyl, or substituted C₁-C₆ alkyl and X is O or NJ1.

In certain embodiments, the Z group is C₁-C₆ alkyl substituted with one or more Xx, wherein each Xx is independently OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, the Z group is C₁-C₆ alkyl substituted with one or more Xx, wherein each Xx is independently halo (e.g., fluoro), hydroxyl, alkoxy (e.g., CH₃O—), substituted alkoxy or azido.

In certain embodiments, the Z group is —CH₂Xx, wherein Xx is OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, the Z group is —CH₂Xx, wherein Xx is halo (e.g., fluoro), hydroxyl, alkoxy (e.g., CH₃O—) or azido.

In certain such embodiments, the Z group is in the (R)-configuration:

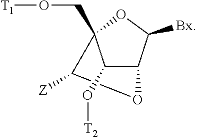

In certain such embodiments, the Z group is in the (S)-configuration:

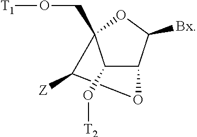

In certain embodiments, each T₁ and T₂ is a hydroxyl protecting group. A preferred list of hydroxyl protecting groups includes benzyl, benzoyl, 2,6-dichlorobenzyl, t-butyldimethylsilyl, t-butyldiphenylsilyl, mesylate, tosylate, dimethoxytrityl (DMT), 9-phenylxanthine-9-yl (Pixyl) and 9 (p-methoxyphenyl)xanthine-9-yl (MOX). In certain embodiments, T₁ is a hydroxyl protecting group selected from acetyl, benzyl, t-butyldimethylsilyl, t-butyldiphenylsilyl and dimethoxytrityl wherein a more preferred hydroxyl protecting group is T₁ is 4,4′-dimethoxytrityl.

In certain embodiments, T₂ is a reactive phosphorus group wherein preferred reactive phosphorus groups include diisopropylcyanoethoxy phosphoramidite and H-phosphonate. In certain embodiments T₁ is 4,4′-dimethoxytrityl and T₂ is diisopropylcyanoethoxy phosphoramidite.

In certain embodiments, the compounds of the invention comprise at least one monomer of the formula:

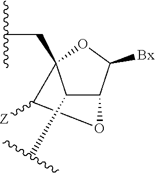

or of the formula:

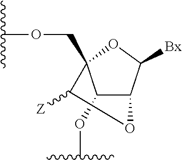

wherein

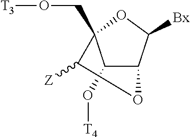

Bx is a heterocyclic base moiety;
T₃ is H, a hydroxyl protecting group, a linked conjugate group or an internucleoside linking group attached to a nucleoside, a nucleotide, an oligonucleoside, an oligonucleotide, a monomeric subunit or an oligomeric compound;
T₄ is H, a hydroxyl protecting group, a linked conjugate group or an internucleoside linking group attached to a nucleoside, a nucleotide, an oligonucleoside, an oligonucleotide, a monomeric subunit or an oligomeric compound;
wherein at least one of T₃ and T₄ is an internucleoside linking group attached to a nucleoside, a nucleotide, an oligonucleoside, an oligonucleotide, a monomeric subunit or an oligomeric compound; and
Z is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, or substituted amide.

In some embodiments, each of the substituted groups, is, independently, mono or poly substituted with optionally protected substituent groups independently selected from halogen, oxo, hydroxyl, OJ1, NJ1J2, SJ1, N3, OC(═X)J1, OC(═X)NJ1J2, NJ3C(═X)NJ1J2 and CN, wherein each J1, J2 and J3 is, independently, H or C1-C6 alkyl, and X is O, S or NJ1.

In some embodiments, each of the substituted groups, is, independently, mono or poly substituted with substituent groups independently selected from halogen, oxo, hydroxyl, OJ1, NJ1J2, SJ1, N3, OC(═X)J1, and NJ3C(═X)NJ1J2, wherein each J1, J2 and J3 is, independently, H or C₁-C₆ alkyl, and X is O or NJ1.

In certain such embodiments, at least one Z is C₁-C₆ alkyl or substituted C₁-C₆ alkyl. In certain embodiments, each Z is, independently, C₁-C₆ alkyl or substituted C₁-C₆ alkyl. In certain embodiments, at least one Z is C₁-C₆ alkyl. In certain embodiments, each Z is, independently, C₁-C₆ alkyl. In certain embodiments, at least one Z is methyl. In certain embodiments, each Z is methyl. In certain embodiments, at least one Z is ethyl. In certain embodiments, each Z is ethyl. In certain embodiments, at least one Z is substituted C₁-C₆ alkyl. In certain embodiments, each Z is, independently, substituted C₁-C₆ alkyl. In certain embodiments, at least one Z is substituted methyl. In certain embodiments, each Z is substituted methyl. In certain embodiments, at least one Z is substituted ethyl. In certain embodiments, each Z is substituted ethyl.

In certain embodiments, at least one substituent group is C₁-C₆ alkoxy (e.g., at least one Z is C₁-C₆ alkyl substituted with one or more C₁-C₆ alkoxy). In another embodiment, each substituent group is, independently, C₁-C₆ alkoxy (e.g., each Z is, independently, C₁-C₆ alkyl substituted with one or more C₁-C₆ alkoxy).

In certain embodiments, at least one C₁-C₆ alkoxy substituent group is CH₃O— (e.g., at least one Z is CH₃OCH₂—). In another embodiment, each C₁-C₆ alkoxy substituent group is CH₃O— (e.g., each Z is CH₃OCH₂—).

In certain embodiments, at least one substituent group is halogen (e.g., at least one Z is C₁-C₆ alkyl substituted with one or more halogen). In certain embodiments, each substituent group is, independently, halogen (e.g., each Z is, independently, C₁-C₆ alkyl substituted with one or more halogen). In certain embodiments, at least one halogen substituent group is fluoro (e.g., at least one Z is CH₂FCH₂—, CHF₂CH₂— or CF₃CH₂—). In certain embodiments, each halo substituent group is fluoro (e.g., each Z is, independently, CH₂FCH₂—, CHF₂CH₂— or CF₃CH₂—).

In certain embodiments, at least one substituent group is hydroxyl (e.g., at least one Z is C1-C6 alkyl substituted with one or more hydroxyl). In certain embodiments, each substituent group is, independently, hydroxyl (e.g., each Z is, independently, C₁-C₆ alkyl substituted with one or more hydroxyl). In certain embodiments, at least one Z is HOCH₂—. In another embodiment, each Z is HOCH₂—.

In certain embodiments, at least one Z is CH₃—, CH₃CH₂—, CH₂OCH₃—, CH₂F— or HOCH₂—. In certain embodiments, each Z is, independently, CH₃—, CH₃CH₂—, CH₂OCH₃—, CH₂F— or HOCH₂—.

In certain embodiments, at least one Z group is C₁-C₆ alkyl substituted with one or more Xx, wherein each Xx is, independently, OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, at least one Z group is C₁-C₆ alkyl substituted with one or more Xx, wherein each Xx is, independently, halo (e.g., fluoro), hydroxyl, alkoxy (e.g., CH₃O—) or azido.

In certain embodiments, each Z group is, independently, C₁-C₆ alkyl substituted with one or more Xx, wherein each Xx is independently OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, each Z group is, independently, C₁-C₆ alkyl substituted with one or more Xx, wherein each Xx is independently halo (e.g., fluoro), hydroxyl, alkoxy (e.g., CH₃O—) or azido.

In certain embodiments, at least one Z group is —CH₂Xx, wherein Xx is OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1 In certain embodiments, at least one Z group is —CH₂Xx, wherein Xx is halo (e.g., fluoro), hydroxyl, alkoxy (e.g., CH₃O—) or azido.

In certain embodiments, each Z group is, independently, —CH₂Xx, wherein each Xx is, independently, OJ1, NJ1J2, SJ1, N3, OC(═X)J1, OC(═X)NJ1J2, NJ3C(═X)NJ1J2 or CN; wherein each J1, J2 and J3 is, independently, H or C₁-C₆ alkyl, and X is O, S or NJ1. In another embodiment, each Z group is, independently, —CH₂Xx, wherein each Xx is, independently, halo (e.g., fluoro), hydroxyl, alkoxy (e.g., CH₃O—) or azido.

In certain embodiments, at least one Z is CH₃—. In another embodiment, each Z is, CH₃—.

In certain embodiments, the Z group of at least one monomer is in the (R)-configuration represented by the formula:

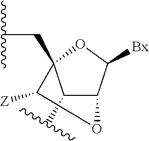

or the formula:

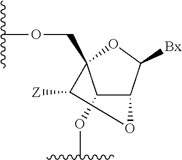

or the formula:

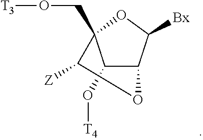

IN certain embodiments, the Z group of each monomer of the formula is in the (R)-configuration.

In certain embodiments, the Z group of at least one monomer is in the (S)-configuration represented by the formula:

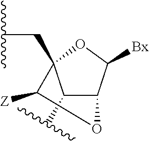

or the formula:

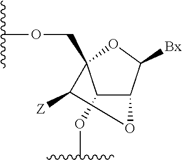

or the formula:

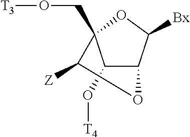

In certain embodiments, the Z group of each monomer of the formula is in the (S)-configuration.

In certain embodiments, T₃ is H or a hydroxyl protecting group. In certain embodiments, T₄ is H or a hydroxyl protecting group. In a further embodiment T₃ is an internucleoside linking group attached to a nucleoside, a nucleotide or a monomeric subunit. In certain embodiments, T₄ is an internucleoside linking group attached to a nucleoside, a nucleotide or a monomeric subunit. In certain embodiments, T₃ is an internucleoside linking group attached to an oligonucleoside or an oligonucleotide. In certain embodiments, T₄ is an internucleoside linking group attached to an oligonucleoside or an oligonucleotide. In certain embodiments, T₃ is an internucleoside linking group attached to an oligomeric compound. In certain embodiments, T₄ is an internucleoside linking group attached to an oligomeric compound. In certain embodiments, at least one of T₃ and T₄ comprises an internucleoside linking group selected from phosphodiester or phosphorothioate.

In certain embodiments, dsRNA agent of the invention comprise at least one region of at least two contiguous monomers of the formula:

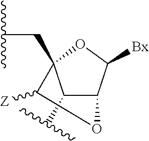

or of the formula:

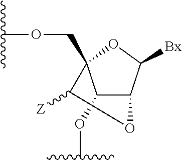

or of the formula:

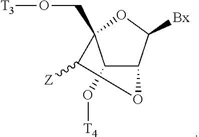

In certain such embodiments, LNAs include, but are not limited to, (A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA, (D) Aminooxy (4′-CH₂—O—N(R)-2′) LNA and (E) Oxyamino (4′-CH₂—N(R)—O-2′) LNA, as depicted below:

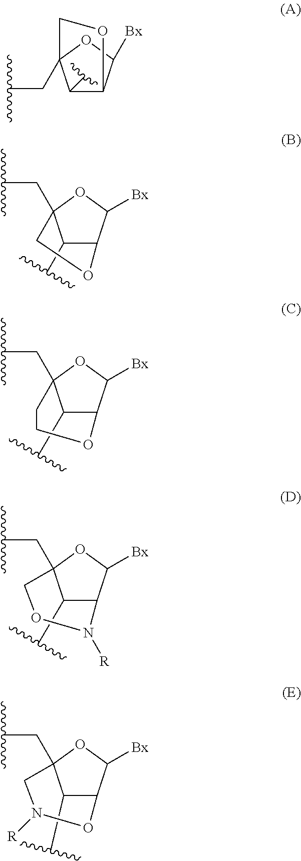

In certain embodiments, the dsRNA agent of the invention comprises at least two regions of at least two contiguous monomers of the above formula. In certain embodiments, the dsRNA agent of the invention comprises a gapped motif. In certain embodiments, the dsRNA agent of the invention comprises at least one region of from about 8 to about 14 contiguous β-D-2′-deoxyribofuranosyl nucleosides. In certain embodiments, the dsRNA agent of the invention comprises at least one region of from about 9 to about 12 contiguous β-D-2′-deoxyribofuranosyl nucleosides.

In certain embodiments, the dsRNA agent of the invention comprises at least one (e.g., 1, 2, 3, 4, 5, 6, 7, 8, 9, 10, 11, 12, 13, 14, 15 or more) comprises at least one (S)-cEt monomer of the formula:

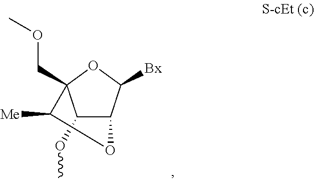

wherein Bx is heterocyclic base moiety.

In certain embodiments, monomers include sugar mimetics. In certain such embodiments, a mimetic is used in place of the sugar or sugar-internucleoside linkage combination, and the nucleobase is maintained for hybridization to a selected target. Representative examples of a sugar mimetics include, but are not limited to, cyclohexenyl or morpholino. Representative examples of a mimetic for a sugar-internucleoside linkage combination include, but are not limited to, peptide nucleic acids (PNA) and morpholino groups linked by uncharged achiral linkages. In some instances a mimetic is used in place of the nucleobase. Representative nucleobase mimetics are well known in the art and include, but are not limited to, tricyclic phenoxazine analogs and universal bases (Berger et al., Nuc Acid Res. 2000, 28:2911-14, incorporated herein by reference). Methods of synthesis of sugar, nucleoside and nucleobase mimetics are well known to those skilled in the art.

### C. Intersugar Linkage Modifications

Described herein are linking groups that link monomers (including, but not limited to, modified and unmodified nucleosides and nucleotides) together, thereby forming an oligomeric compound, e.g., an oligonucleotide. Such linking groups are also referred to as intersugar linkage. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH2-), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Modified linkages, compared to natural phosphodiester linkages, can be used to alter, typically increase, nuclease resistance of the oligonucleotides. In certain embodiments, linkages having a chiral atom can be prepared as racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The phosphate group in the linking group can be modified by replacing one of the oxygens with a different substituent. One result of this modification can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the sugar of the monomer), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “iron-phosphodiester intersugar linkage” or “non-phosphodiester linker.”

In certain embodiments, the phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(O)—N(H)-5′) and amide-4 (3′-CH₂—N(H)—C(O)-5′)), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′ N(H)—C(═O)—CH₂—S—C5′, C3′-O—P(O)—O—SS—C5′, C3′-CH₂—NH—NH—C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides, carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

In some embodiments, the dsRNA agent of the invention comprises at least one (e.g., 1, 2, 3, 4, 5, 6, 7, 8, 9, 10, 11, 12, 13, 14, 15 or more and upto including all) modified or nonphosphodiester linkages. In some embodiments, the dsRNA agent of the invention comprises at least one (e.g., 1, 2, 3, 4, 5, 6, 7, 8, 9, 10, 11, 12, 13, 14, 15 or more and upto including all) phosphorothioate linkages.

The dsRNA agent of the inventions can also be constructed wherein the phosphate linker and the sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backnone-extended pyrrolidine PNA (bepPNA) nucleoside surrogates. A preferred surrogate is a PNA surrogate.

The dsRNA agent of the inventions described herein can contain one or more asymmetric centers and thus give rise to enantiomers, diastereomers, and other stereoisomeric configurations that may be defined, in terms of absolute stereochemistry, as (R) or (S), such as for sugar anomers, or as (D) or (L) such as for amino acids et al. Included in the dsRNA agent of the inventions provided herein are all such possible isomers, as well as their racemic and optically pure forms.

### D. Terminal Modifications

In some embodiments, the dsRNA agent further comprises a phosphate or phosphate mimic at the 5′-end of the antisense strand. In one embodiment, the phosphate mimic is a 5′-vinyl phosphonate (VP).

In some embodiments, the 5′-end of the antisense strand of the dsRNA agent does not contain a 5′-vinyl phosphonate (VP).

Ends of the iRNA agent of the invention can be modified. Such modifications can be at one end or both ends. For example, the 3′ and/or 5′ ends of an iRNA can be conjugated to other functional molecular entities such as labeling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

When a linker/phosphate-functional molecular entity-linker/phosphate array is interposed between two strands of a double stranded oligomeric compound, this array can substitute for a hairpin loop in a hairpin-type oligomeric compound.

Terminal modifications useful for modulating activity include modification of the 5′ end of iRNAs with phosphate or phosphate analogs. In certain embodiments, the 5′end of an iRNA is phosphorylated or includes a phosphoryl analog. Exemplary 5′-phosphate modifications include those which are compatible with RISC mediated gene silencing. Modifications at the 5′-terminal end can also be useful in stimulating or inhibiting the immune system of a subject. In some embodiments, the 5′-end of the oligomeric compound comprises the modification

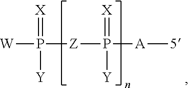

wherein W, X and Y are each independently selected from the group consisting of 0, OR (R is hydrogen, alkyl, aryl), S, Se, BR₃ (R is hydrogen, alkyl, aryl), BH₃⁻, C (i.e. an alkyl group, an aryl group, etc. . . . ), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments, n is 1 or 2. It is understood that A is replacing the oxygen linked to 5′ carbon of sugar. When n is 0, W and Y together with the P to which they are attached can form an optionally substituted 5-8 membered heterocyclic, wherein W an Y are each independently O, S, NR′ or alkylene. Preferably the heterocyclic is substituted with an aryl or heteroaryl. In some embodiments, one or both hydrogen on C5′ of the 5′-terminal nucleotides are replaced with a halogen, e.g., F.

Exemplary 5′-modifications include, but are not limited to, 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O-5′). Other 5′-modification include 5′-alkylphosphonates (R(OH)(O)P—O-5′, R=alkyl, e.g., methyl, ethyl, isopropyl, propyl, etc. . . . ), 5′-alkyletherphosphonates (R(OH)(O)P—O-5′, R=alkylether, e.g., methoxymethyl (CH₂OMe), ethoxymethyl, etc. . . . ). Other exemplary 5′-modifications include where Z is optionally substituted alkyl at least once, e.g., ((HO)₂(X)P—O[—(CH₂)ₙ—O—P(X)(OH)—O]_{b}-5′, ((HO)₂(X)P—O[—(CH₂)ₙ—P(X)(OH)—O]_{b}-5′, ((HO)2(X)P—[—(CH₂)ₙ—O—P(X)(OH)—O]_{b}-5′; dialkyl terminal phosphates and phosphate mimics: HO[—(CH₂)ₙ—O—P(X)(OH)—O]_{b}-5′, H₂N[—(CH₂)ₙ—O—P(X)(OH)—O]_{b}-5′, H[—(CH₂)ₙ—O—P(X)(OH)—O]_{b}-5′, Me₂N[—(CH₂), —O—P(X)(OH)—O]_{b}-5′, HO[—(CH₂)ₙ—P(X)(OH)—O]_{b}-5′, H₂N[—(CH₂)ₙ—P(X)(OH)—O]_{b}-5′, H[—(CH₂), —P(X)(OH)—O]_{b}-5′, Me₂N[—(CH₂)ₙ—P(X)(OH)—O]_{b}-5′, wherein a and b are each independently 1-10. Other embodiments, include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

### E. Thermally Destabilizing Modifications

The compounds of the invention, such as iRNAs or dsRNA agents, can be optimized for RNA interference by increasing the propensity of the iRNA duplex to disassociate or melt (decreasing the free energy of duplex association) by introducing a thermally destabilizing modification in the sense strand at a site opposite to the seed region of the antisense strand (i.e., at positions 2-8 of the 5′-end of the antisense strand, or at positions 2-9 of the 5′-end of the antisense strand). This modification can increase the propensity of the duplex to disassociate or melt in the seed region of the antisense strand.

The thermally destabilizing modifications can include abasic modification; mismatch with the opposing nucleotide in the opposing strand; and sugar modification such as 2′-deoxy modification or acyclic nucleotide, e.g., unlocked nucleic acids (UNA) or glycerol nucleic acid (GNA).

Exemplified abasic modifications are:

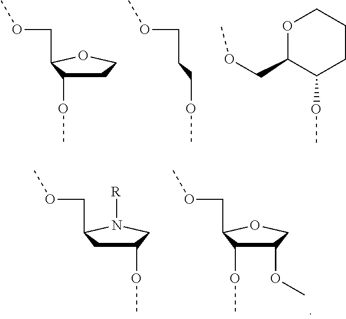

Exemplified sugar modifications are:

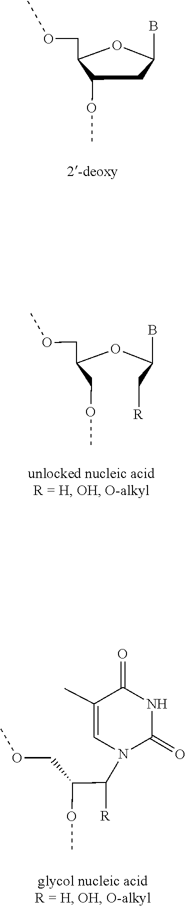

The term “acyclic nucleotide” refers to any nucleotide having an acyclic ribose sugar, for example, where any of bonds between the ribose carbons (e.g., C1′-C2′, C2′-C3′, C3′-C4′, C4′-O4′, or C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

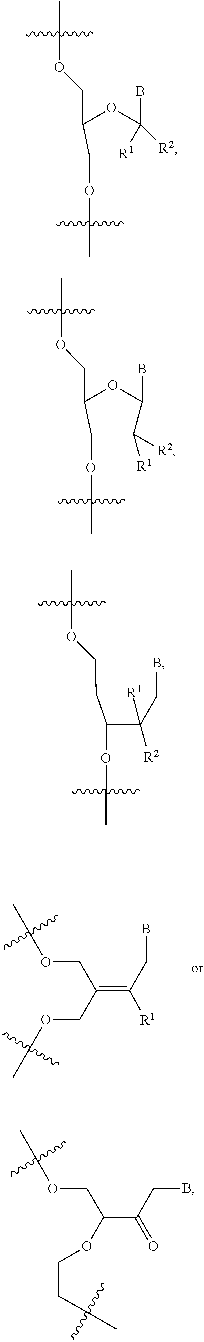

wherein B is a modified or unmodified nucleobase, R¹ and R² independently are H, halogen, OR₃, or alkyl; and R₃ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4′ being removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

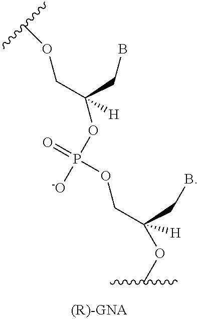

The thermally destabilizing modification can be mismatches (i.e., noncomplementary base pairs) between the thermally destabilizing nucleotide and the opposing nucleotide in the opposite strand within the dsRNA duplex. Exemplary mismatch basepairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the compounds of the invention, such as siRNA or iRNA agent, contains at least one nucleobase in the mismatch pairing that is a 2′-deoxy nucleobase; e.g., the 2′-deoxy nucleobase is in the sense strand.

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

Nucleobase modifications with impaired or completely abolished capability to form hydrogen bonds with bases in the opposite strand have been evaluated for destabilization of the central region of the dsRNA duplex as described in WO 2010/0011895, which is herein incorporated by reference in its entirety. Exemplary nucleobase modifications are:

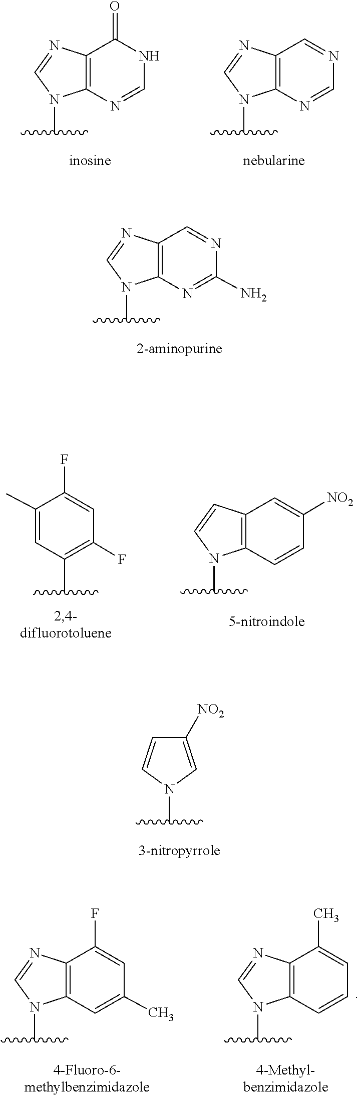

Exemplary phosphate modifications known to decrease the thermal stability of dsRNA duplexes compared to natural phosphodiester linkages are:

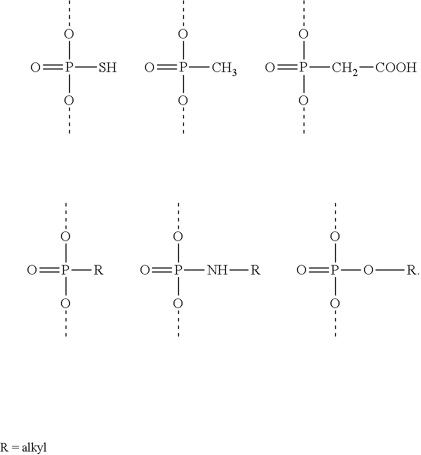

In some embodiments, compounds of the invention can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, compounds of the invention can comprise L sugars (e.g., L ribose, L-arabinose with 2′-H, 2′-OH and 2′-OMe). For example, these L sugar modifications can be used to promote nuclease resistance or to inhibit binding of the sense to the antisense strand, or can be used at the 5′ end of the sense strand to avoid sense strand activation by RISC.

In one embodiment the iRNA agent of the invention is conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and decalin; preferably, the acyclic group is selected from serinol backbone or diethanolamine backbone.

In some embodiments, at least one strand of the iRNA agent of the invention disclosed herein is 5′ phosphorylated or includes a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7 methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g. 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g. RP(OH)(O)—O-5′-, 5′-alkenylphosphonates (i.e. vinyl, substituted vinyl), (OH)₂(O)P-5′-CH₂—), 5′-alkyletherphosphonates (R=alkylether=nethoxymethyl (MeOCH₂—), ethoxymethyl, etc., e.g. RP(OH)(O)—O-5′-).

### IV. Modified RNAi Agents of the Invention Comprising Motifs

In certain aspects of the disclosure, the double-stranded RNAi agents of the disclosure include agents with chemical modifications as disclosed, for example, in U.S. Pat. Nos. 9,796,974 and 10,668,170, and U.S. Patent Publication Nos. 2014/288158, 2018/008724, 2019/038768, and 2020/353097, the entire contents of each of which are incorporated herein by reference. As shown therein and in PCT Publication No. WO 2013/074974 (the entire contents of which are incorporated by reference), one or more motifs of three identical modifications on three consecutive nucleotides may be introduced into a sense strand or antisense strand of an RNAi agent, particularly at or near the cleavage site. In some embodiments, the sense strand and antisense strand of the RNAi agent may otherwise be completely modified. The introduction of these motifs interrupts the modification pattern, if present, of the sense or antisense strand. The RNAi agent may be optionally modified with a (5)-glycol nucleic acid (GNA) modification, for instance on one or more residues of the antisense strand.

In one embodiment, the iRNA agent of the invention is a double ended bluntmer of 19 nt in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, 13 from the 5′end.

In one embodiment, the iRNA agent of the invention is a double ended bluntmer of 20 nt in length, wherein the sense strand contains at least one motif of three 2′-F modifications on three consecutive nucleotides at positions 8, 9, 10 from the 5′end. The antisense strand contains at least one motif of three 2′-O-methyl modifications on three consecutive nucleotides at positions 11, 12, 13 from the 5′end.

In one embodiment, the iRNA agent of the invention is a double ended bluntmer of 21 nt in length, wherein the sense strand contains at least one motif of three 2′-F modifications on three consecutive nucleotides at positions 9, 10, 11 from the 5′end. The antisense strand contains at least one motif of three 2′-O-methyl modifications on three consecutive nucleotides at positions 11, 12, 13 from the 5′end.

In one embodiment, the iRNA agent of the invention comprises a 21 nucleotides (nt) sense strand and a 23 nucleotides (nt) antisense, wherein the sense strand contains at least one motif of three 2′-F modifications on three consecutive nucleotides at positions 9, 10, 11 from the 5′end; the antisense strand contains at least one motif of three 2′-O-methyl modifications on three consecutive nucleotides at positions 11, 12, 13 from the 5′end, wherein one end of the iRNA is blunt, while the other end is comprises a 2 nt overhang. Preferably, the 2 nt overhang is at the 3′-end of the antisense. Optionally, the iRNA agent further comprises a ligand (e.g., GalNAc₃).

In one embodiment, the iRNA agent of the invention comprises a sense and antisense strands, wherein: the sense strand is 25-30 nucleotide residues in length, wherein starting from the 5′ terminal nucleotide (position 1) positions 1 to 23 of said first strand comprise at least 8 ribonucleotides; antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when said double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the iRNA agent of the invention comprises a sense and antisense strands, wherein said iRNA agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein said 3′ end of said first strand and said 5′ end of said second strand form a blunt end and said second strand is 1-4 nucleotides longer at its 3′ end than the first strand, wherein the duplex region which is at least 25 nucleotides in length, and said second strand is sufficiently complementary to a target mRNA along at least 19 nt of said second strand length to reduce target gene expression when said iRNA agent is introduced into a mammalian cell, and wherein dicer cleavage of said iRNA preferentially results in an siRNA comprising said 3′ end of said second strand, thereby reducing expression of the target gene in the mammal. Optionally, the iRNA agent further comprises a ligand (e.g., GalNAc₃).

In one embodiment, the sense strand of the iRNA agent contains at least one motif of three identical modifications on three consecutive nucleotides, where one of the motifs occurs at the cleavage site in the sense strand. For instance, the sense strand can contain at least one motif of three 2′-F modifications on three consecutive nucleotides within 7-15 positions from the 5′end.

In one embodiment, the antisense strand of the iRNA agent can also contain at least one motif of three identical modifications on three consecutive nucleotides, where one of the motifs occurs at or near the cleavage site in the antisense strand. For instance, the antisense strand can contain at least one motif of three 2′-O-methyl modifications on three consecutive nucleotides within 9-15 positions from the 5′end.

For iRNA agent having a duplex region of 17-23 nt in length, the cleavage site of the antisense strand is typically around the 10, 11 and 12 positions from the 5′-end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1^{st} nucleotide from the 5′-end of the antisense strand, or, the count starting from the 1^{st} paired nucleotide within the duplex region from the 5′-end of the antisense strand. The cleavage site in the antisense strand may also change according to the length of the duplex region of the iRNA from the 5′-end.

In some embodiments, the iRNA agent comprises a sense strand and antisense strand each having 14 to 30 nucleotides, wherein the sense strand contains at least two motifs of three identical modifications on three consecutive nucleotides, where at least one of the motifs occurs at or near the cleavage site within the strand and at least one of the motifs occurs at another portion of the strand that is separated from the motif at the cleavage site by at least one nucleotide. In one embodiment, the antisense strand also contains at least one motif of three identical modifications on three consecutive nucleotides, where at least one of the motifs occurs at or near the cleavage site within the strand. The modification in the motif occurring at or near the cleavage site in the sense strand is different than the modification in the motif occurring at or near the cleavage site in the antisense strand.

In some embodiments, the iRNA agent comprises a sense strand and antisense strand each having 14 to 30 nucleotides, wherein the sense strand contains at least one motif of three 2′-F modifications on three consecutive nucleotides, where at least one of the motifs occurs at or near the cleavage site in the strand. In one embodiment, the antisense strand also contains at least one motif of three 2′-O-methyl modifications on three consecutive nucleotides at or near the cleavage site.

In some embodiments, the iRNA agent comprises a sense strand and antisense strand each having 14 to 30 nucleotides, wherein the sense strand contains at least one motif of three 2′-F modifications on three consecutive nucleotides at positions 9, 10, 11 from the 5′end, and wherein the antisense strand contains at least one motif of three 2′-O-methyl modifications on three consecutive nucleotides at positions 11, 12, 13 from the 5′end.

In one embodiment, the iRNA agent of the invention comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the iRNA agent of the invention comprises at least one of the first 1, 2, 3, 4, or 5 base pairs within the duplex regions from the 5′-end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′-end of the antisense strand is an AU base pair. For example, the first base pair within the duplex region from the 5′-end of the antisense strand is an AU base pair.

In another embodiment, the nucleotide at the 3′-end of the sense strand is deoxythimidine (dT). In another embodiment, the nucleotide at the 3′-end of the antisense strand is deoxythimidine (dT). In one embodiment, there is a short sequence of deoxythimidine nucleotides, for example, two dT nucleotides on the 3′-end of the sense or antisense strand.

In certain embodiments, the compositions and methods of the disclosure include a vinyl phosphonate (VP) modification of an RNAi agent as described herein. In exemplary embodiments, a 5′-vinyl phosphonate modified nucleotide of the disclosure has the structure:

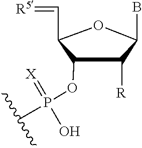

wherein X is O or S;
R is hydrogen, hydroxy, fluoro, or C₁₋₂₀alkoxy (e.g., methoxy or n-hexadecyloxy);
R^{5′} is (H)—P(O)(OH)₂ and the double bond between the C5′ carbon and R^{5′} is in the E or Z orientation (e.g., E orientation); and
B is a nucleobase or a modified nucleobase, optionally where B is adenine, guanine, cytosine, thymine, or uracil.

A vinyl phosphonate of the instant disclosure may be attached to either the antisense or the sense strand of a dsRNA of the disclosure. In certain embodiments, a vinyl phosphonate of the instant disclosure is attached to the antisense strand of a dsRNA, optionally at the 5′ end of the antisense strand of the dsRNA.

Vinyl phosphate modifications are also contemplated for the compositions and methods of the instant disclosure. An exemplary vinyl phosphate structure includes the preceding structure, where R5′ is (H)—OP(O)(OH)₂ and the double bond between the C5′ carbon and R5′ is in the E or Z orientation (e.g., E orientation).

In one aspect, the invention relates to a double-stranded RNA (dsRNA) agent for inhibiting the expression of a target gene having reduced off-target effects as described in U.S. Pat. Nos. 10,233,448, 10,612,024, and 10,612,027, and U.S. Patent Publication Nos. 2017/275626, 2019/241891, 2019/241893, and 2021/017519, the entire contents of each of which are incorporated herein by reference. As exemplified therein, a motif comprising, e.g., a thermally destabilizing nucleotide, e.g., i) a nucleotide that forms a mismatch pair with the opposing nucleotide in the antisense strand, ii) a nucleotide having an abasic modification, and/or iii) a nucleotide having a sugar modification, and placed at a site opposite to the seed region (positions 2-8) may be introduced into the sense strand.

In one embodiment, the dsRNA agent of the invention does not contain any 2′-F modification.

In one embodiment, the sense strand and/or antisense strand of the dsRNA agent comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In one embodiment, each of the sense and antisense strands of the dsRNA agent has 15-30 nucleotides. In one example, the sense strand has 19-22 nucleotides, and the antisense strand has 19-25 nucleotides. In another example, the sense strand has 21 nucleotides, and the antisense strand has 23 nucleotides.

In one embodiment, the nucleotide at position 1 of the 5′-end of the antisense strand in the duplex is selected from the group consisting of A, dA, dU, U, and dT. In one embodiment, at least one of the first, second, and third base pair from the 5′-end of the antisense strand is an AU base pair.

In one embodiment, the antisense strand of the dsRNA agent of the invention is 100% complementary to a target RNA to hybridize thereto and inhibits its expression through RNA interference. In another embodiment, the antisense strand of the dsRNA agent of the invention is at least 95%, at least 90%, at least 85%, at least 80%, at least 75%, at least 70%, at least 65%, at least 60%, at least 55%, or at least 50% complementary to a target RNA.

In one aspect, the invention relates to a dsRNA agent as defined herein capable of inhibiting the expression of a target gene. The dsRNA agent comprises a sense strand and an antisense strand, each strand having 14 to 40 nucleotides. The sense strand contains at least one thermally destabilizing nucleotide, wherein at least one of said thermally destabilizing nucleotide occurs at or near the site that is opposite to the seed region of the antisense strand (i.e. at position 2-8 of the 5′-end of the antisense strand, or at positions 2-9 of the 5′-end of the antisense strand). Each of the embodiments and aspects described in this specification relating to the dsRNA represented by formula (I) can also apply to the dsRNA containing the thermally destabilizing nucleotide.

The thermally destabilizing nucleotide can occur, for example, between positions 14-17 of the 5′-end of the sense strand when the sense strand is 21 nucleotides in length. The antisense strand contains at least two modified nucleic acids that are smaller than a sterically demanding 2′-OMe modification. Preferably, the two modified nucleic acids that are smaller than a sterically demanding 2′-OMe are separated by 11 nucleotides in length. For example, the two modified nucleic acids are at positions 2 and 14 of the 5′end of the antisense strand.

In one embodiment, the dsRNA agent further comprises at least one ASGPR ligand. For example, the ASGPR ligand is one or more GalNAc derivatives attached through a bivalent or trivalent branched linker, such as:

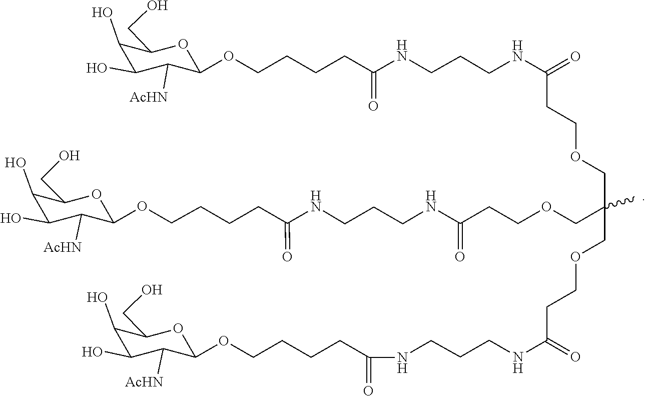 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In one example, the ASGPR ligand is attached to the 3′ end of the sense strand.

For example, the dsRNA agent as defined herein can comprise i) a phosphorus-containing group at the 5′-end of the sense strand or antisense strand; ii) with two phosphorothioate internucleotide linkage modifications within position 1-5 of the sense strand (counting from the 5′ end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand); and iii) a ligand, such as a ASGPR ligand (e.g., one or more GalNAc derivatives) at 5′-end or 3′-end of the sense strand or antisense strand. For instance, the ligand may be at the 3′-end of the sense strand.

In a particular embodiment, the dsRNA agents of the present invention comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker; and
   (iii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end);
   and
(b) an antisense strand having:
   (i) a length of 23 nucleotides;
   (ii) 2′-OMe modifications at positions 1, 3, 5, 9, 11 to 13, 15, 17, 19, 21, and 23, and 2′F modifications at positions 2, 4, 6 to 8, 10, 14, 16, 18, 20, and 22 (counting from the 5′ end); and
   (iii) phosphorothioate internucleotide linkages between nucleotide positions 21 and 22, and between nucleotide positions 22 and 23 (counting from the 5′ end);
      wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker;
   (iii) 2′-F modifications at positions 1, 3, 5, 7, 9 to 11, 13, 15, 17, 19, and 21, and 2′-OMe modifications at positions 2, 4, 6, 8, 12, 14, 16, 18, and 20 (counting from the 5′ end); and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker;
   (iii) 2′-OMe modifications at positions 1 to 6, 8, 10, and 12 to 21, 2′-F modifications at positions 7, and 9, and a desoxy-nucleotide (e.g. dT) at position 11 (counting from the 5′ end); and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3, 7, 9, 11, 13, 15, 17, and 19 to 23, and 2′-F modifications at positions 2, 4 to 6, 8, 10, 12, 14, 16, and 18 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker;
   (iii) 2′-OMe modifications at positions 1 to 6, 8, 10, 12, 14, and 16 to 21, and 2′-F modifications at positions 7, 9, 11, 13, and 15; and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 5, 7, 9, 11, 13, 15, 17, 19, and 21 to 23, and 2′-F modifications at positions 2 to 4, 6, 8, 10, 12, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker;
   (iii) 2′-OMe modifications at positions 1 to 9, and 12 to 21, and 2′-F modifications at positions 10, and 11; and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′-F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker;
   (iii) 2′-F modifications at positions 1, 3, 5, 7, 9 to 11, and 13, and 2′-OMe modifications at positions 2, 4, 6, 8, 12, and 14 to 21; and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3, 5 to 7, 9, 11 to 13, 15, 17 to 19, and 21 to 23, and 2′-F modifications at positions 2, 4, 8, 10, 14, 16, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker;
   (iii) 2′-OMe modifications at positions 1, 2, 4, 6, 8, 12, 14, 15, 17, and 19 to 21, and 2′-F modifications at positions 3, 5, 7, 9 to 11, 13, 16, and 18; and
   (iv) phosphorothioate internucleotide linkages between nucleotide positions 1 and 2, and between nucleotide positions 2 and 3 (counting from the 5′ end);
   and
(b) an antisense strand having:
   (i) a length of 25 nucleotides;
   (ii) 2′-OMe modifications at positions 1, 4, 6, 7, 9, 11 to 13, 15, 17, and 19 to 23, 2′-F modifications at positions 2, 3, 5, 8, 10, 14, 16, and 18, and desoxy-nucleotides (e.g. dT) at positions 24 and 25 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a four nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker;
   (iii) 2′-OMe modifications at positions 1 to 6, 8, and 12 to 21, and 2′-F modifications at positions 7, and 9 to 11; and
   (iv) phosphorothioate internucleotide linkages between nucleotide positions 1 and 2, and between nucleotide positions 2 and 3 (counting from the 5′ end); and
(b) an antisense strand having:
   (i) a length of 23 nucleotides;
   (ii) 2′-OMe modifications at positions 1, 3 to 5, 7, 8, 10 to 13, 15, and 17 to 23, and 2′-F modifications at positions 2, 6, 9, 14, and 16 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 21 nucleotides;
   (ii) optionally an ASGPR ligand attached to the 3′-end, wherein said ASGPR ligand comprises three GalNAc derivatives attached through a trivalent branched linker;
   (iii) 2′-OMe modifications at positions 1 to 6, 8, and 12 to 21, and 2′-F modifications at positions 7, and 9 to 11; and
   (iv) phosphorothioate internucleotide linkages between nucleotide positions 1 and 2, and between nucleotide positions 2 and 3 (counting from the 5′ end);
   and
(b) an antisense strand having:
   (i) a length of 23 nucleotides;
   (ii) 2′-OMe modifications at positions 1, 3 to 5, 7, 10 to 13, 15, and 17 to 23, and 2′-F modifications at positions 2, 6, 8, 9, 14, and 16 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, the dsRNA agents of the present invention comprise:
(a) a sense strand having:
   (i) a length of 19 nucleotides;
   (ii) optionally an ASGPR ligand attached to the 3′-end, wherein said ASGPR ligand comprises three GalNAc derivatives attached through a trivalent branched linker;
   (iii) 2′-OMe modifications at positions 1 to 4, 6, and 10 to 19, and 2′-F modifications at positions 5, and 7 to 9; and
   (iv) phosphorothioate internucleotide linkages between nucleotide positions 1 and 2, and between nucleotide positions 2 and 3 (counting from the 5′ end);
   and
(b) an antisense strand having:
   (i) a length of 21 nucleotides;
   (ii) 2′-OMe modifications at positions 1, 3 to 5, 7, 10 to 13, 15, and 17 to 21, and 2′-F modifications at positions 2, 6, 8, 9, 14, and 16 (counting from the 5′ end); and
   (iii) phosphorothioate internucleotide linkages between nucleotide positions 1 and 2, between nucleotide positions 2 and 3, between nucleotide positions 19 and 20, and between nucleotide positions 20 and 21 (counting from the 5′ end);
wherein the dsRNA agents have a two nucleotide overhang at the 3′-end of the antisense strand, and a blunt end at the 5′-end of the antisense strand.

In one embodiment, the dsRNA agents of the present invention comprise:
(a) a sense strand having:
   (i) a length of 18-23 nucleotides;
   (ii) three consecutive 2′-F modifications at positions 7-15; and
(b) an antisense strand having:
   (i) a length of 18-23 nucleotides;
   (ii) at least 2′-F modifications anywhere on the strand; and
   (iii) at least two phosphorothioate internucleotide linkages at the first five nucleotides (counting from the 5′ end);
wherein the dsRNA agents have one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the dsRNA agents of the present invention comprise:
(a) a sense strand having:
   (i) a length of 18-23 nucleotides;
   (ii) less than four 2′-F modifications;
(b) an antisense strand having:
   (i) a length of 18-23 nucleotides;
   (ii) at less than twelve 2′-F modification; and
   (iii) at least two phosphorothioate internucleotide linkages at the first five nucleotides (counting from the 5′ end);
wherein the dsRNA agents have one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the dsRNA agents of the present invention comprise:
(a) a sense strand having:
   (i) a length of 19-35 nucleotides;
   (ii) less than four 2′-F modifications;
(b) an antisense strand having:
   (i) a length of 19-35 nucleotides;
   (ii) at less than twelve 2′-F modification; and
   (iii) at least two phosphorothioate internucleotide linkages at the first five nucleotides (counting from the 5′ end);
wherein the duplex region is between 19 to 25 base pairs (preferably 19, 20, 21 or 22); and wherein the dsRNA agents have one or more lipophilic moieties conjugated to one or more positions on at least one strand; and either have two nucleotides overhang at the 3′-end of the antisense strand, and a blunt end at the 5′-end of the antisense strand; or blunt end both ends of the duplex.

In one embodiment, the dsRNA agents of the present invention comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have one or more lipophilic moieties conjugated to one or more positions on at least one strand; and wherein the dsRNA agents have less than 20%, less than 15% and less than 10% non-natural nucleotide.

Examples of non-natural nucleotide includes acyclic nucleotides, LNA, HNA, CeNA, 2′-methoxyethyl, 2′-O-allyl, 2′-C-allyl, 2′-deoxy, 2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F, and others.

In one embodiment, the dsRNA agents of the present invention comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have one or more lipophilic moieties conjugated to one or more positions on at least one strand; and wherein the dsRNA agents have greater than 80%, greater than 85% and greater than 90% natural nucleotide, such as 2′-OH, 2′-deoxy and 2′-OMe are natural nucleotides.

In one embodiment, the dsRNA agents of the present invention comprise a sense strand and antisense strands having a length of 15-30 nucleotides; at least two phosphorothioate internucleotide linkages at the first five nucleotides on the antisense strand (counting from the 5′ end); wherein the duplex region is between 19 to 25 base pairs (preferably 19, 20, 21 or 22); wherein the dsRNA agents have one or more lipophilic moieties conjugated to one or more positions on at least one strand; and wherein the dsRNA agents have 100% natural nucleotide, such as 2′-OH, 2′-deoxy and 2′-OMe are natural nucleotides.

Various publications described multimeric siRNA and can all be used with the iRNA of the invention. Such publications include WO2007/091269, U.S. Pat. No. 7,858,769, WO2010/141511, WO2007/117686, WO2009/014887 and WO2011/031520, which are hereby incorporated by reference in their entirety.

In some embodiments, 100%, 95%, 90%, 85%, 80%, 75%, 70%, 65%, 60%, 55%, 50%, 45%, 40%, 35% or 30% of the iRNA agent of the invention is modified.

In some embodiments, each of the sense and antisense strands of the iRNA agent is independently modified with acyclic nucleotides, LNA, HNA, CeNA, 2′-methoxyethyl, 2′-O-methyl, 2′-O-allyl, 2′-C-allyl, 2′-deoxy, 2′-fluoro, 2′-O—N-methylacetamido (2′-O-NMA), a 2′-O-dimethylaminoethoxyethyl (2′-O-DMAEOE), 2′-O-aminopropyl (2′-O-AP), or 2′-ara-F.

In some embodiments, each of the sense and antisense strands of the iRNA agent contains at least two different modifications.

In some embodiments, the dsRNA agent of the invention of the invention does not contain any 2′-F modification.

In some embodiments, the dsRNA agent of the invention contains one, two, three, four, five, six, seven, eight, nine, ten, eleven or twelve 2′-F modification(s). In one example, dsRNA agent of the invention contains nine or ten 2′-F modifications.

The iRNA agent of the invention may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In one embodiment, the iRNA comprises the phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments, the sense strand and/or antisense strand of the iRNA agent comprises one or more blocks of phosphorothioate or methylphosphonate internucleotide linkages. In one example, the sense strand comprises one block of two phosphorothioate or methylphosphonate internucleotide linkages. In one example, the antisense strand comprises two blocks of two phosphorothioate or methylphosphonate internucleotide linkages. For example, the two blocks of phosphorothioate or methylphosphonate internucleotide linkages are separated by 16-18 phosphate internucleotide linkages.

In some embodiments, the antisense strand of the iRNA agent of the invention is 100% complementary to a target RNA to hybridize thereto and inhibits its expression through RNA interference. In another embodiment, the antisense strand of the iRNA agent of the invention is at least 95%, at least 90%, at least 85%, at least 80%, at least 75%, at least 70%, at least 65%, at least 60%, at least 55%, or at least 50% complementary to a target RNA.

In one aspect, the invention relates to a iRNA agent capable of inhibiting the expression of a target gene. The iRNA agent comprises a sense strand and an antisense strand, each strand having 14 to 40 nucleotides. The sense strand contains at least one thermally destabilizing nucleotide, wherein at at least one said thermally destabilizing nucleotide occurs at or near the site that is opposite to the seed region of the antisense strand (i.e. at position 2-8 of the 5′-end of the antisense strand, or at positions 2-9 of the 5′-end of the antisense strand). For example, the thermally destabilizing nucleotide occurs between positions 14-17 of the 5′-end of the sense strand when the sense strand is 21 nucleotides in length. The antisense strand contains at least two modified nucleic acids that are smaller than a sterically demanding 2′-OMe modification. Preferably, the two modified nucleic acids that is smaller than a sterically demanding 2′-OMe are separated by 11 nucleotides in length. For example, the two modified nucleic acids are at positions 2 and 14 of the 5′end of the antisense strand.

In some embodiments, the compound of the invention disclosed herein is a miRNA mimic. In one design, miRNA mimics are double stranded molecules (e.g., with a duplex region of between about 16 and about 31 nucleotides in length) and contain one or more sequences that have identity with the mature strand of a given miRNA. Double-stranded miRNA mimics have designs similar to as described above for double-stranded iRNAs. In some embodiments, a miRNA mimic comprises a duplex region of between 16 and 31 nucleotides and one or more of the following chemical modification patterns: the sense strand contains 2′-O-methyl modifications of nucleotides 1 and 2 (counting from the 5′ end of the sense oligonucleotide), and all of the Cs and Us; the antisense strand modifications can comprise 2′ F modification of all of the Cs and Us, phosphorylation of the 5′ end of the oligonucleotide, and stabilized internucleotide linkages associated with a 2 nucleotide 3′ overhang.

### V. C₂₂ Hydrocarbon Chains

As described In U.S. Provisional Application No. 63/255,984, filed on Oct. 15, 2021 (the entire contents of which are incorporated herein by reference), including a C₂₂ hydrocarbon chain, e.g., saturated or unsaturated, on one or more internal position(s) of the dsRNA agent increases lipophilicity of the dsRNA agent and provides optimal hydrophobicity for the enhanced in vivo delivery of dsRNA to, e.g., muscle tissue and/or adipose tissue.

One way to characterize lipophilicity is by the octanol-water partition coefficient, log K_{ow}, where K_{ow} is the ratio of a chemical's concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical which are calculated using first-principle or empirical methods (see, for example, Tetko et al., J. Chem. Inf. Comput. Sci. 41:1407-21 (2001), which is incorporated herein by reference in its entirety). It provides a thermodynamic measure of the tendency of the substance to prefer a non-aqueous or oily milieu rather than water (i.e. its hydrophilic/lipophilic balance). In principle, a chemical substance is lipophilic in character when its log K_{ow} exceeds 0. Typically, the lipophilic moiety possesses a log K_{ow} exceeding 1, exceeding 1.5, exceeding 2, exceeding 3, exceeding 4, exceeding 5, or exceeding 10. For instance, the log K_{ow} of 6-amino hexanol, for instance, is predicted to be approximately 0.7. Using the same method, the log K_{ow} of cholesteryl N-(hexan-6-ol) carbamate is predicted to be 10.7.

The lipophilicity of a molecule can change with respect to the functional group it carries. For instance, adding a hydroxyl group or amine group to the end of a C₂₂ hydrocarbon chain can increase or decrease the partition coefficient (e.g., log K_{ow}) value of the C₂₂ hydrocarbon chain.

Alternatively, the hydrophobicity of the dsRNA agent, conjugated to one or more C₂₂ hydrocarbon chains, can be measured by its protein binding characteristics. For instance, the unbound fraction in the plasma protein binding assay of the dsRNA agent can be determined to positively correlate to the relative hydrophobicity of the dsRNA agent, which can positively correlate to the silencing activity of the dsRNA agent.

In one embodiment, the plasma protein binding assay determined is an electrophoretic mobility shift assay (EMSA) using human serum albumin protein. The hydrophobicity of the dsRNA agent, measured by fraction of unbound dsRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of siRNA.

In certain embodiments, the one or more C₂₂ hydrocarbon chains is an aliphatic, alicyclic, or polyalicyclic compound is an aliphatic, cyclic such as alicyclic, or polycyclic such as polyalicyclic compound. The hydrocarbon chain may comprise various substituents and/or one or more heteroatoms, such as an oxygen or nitrogen atom.

The one or more C₂₂ hydrocarbon chains may be attached to the iRNA agent by any method known in the art, including via a functional grouping already present in the lipophilic moiety or introduced into the iRNA agent, such as a hydroxy group (e.g., —CO—CH₂—OH). The functional groups already present in the C₂₂ hydrocarbon chain or introduced into the dsRNA agent include, but are not limited to, hydroxyl, amine, carboxylic acid, sulfonate, phosphate, thiol, azide, and alkyne.

Conjugation of the dsRNA agent and the C₂₂ hydrocarbon chain may occur, for example, through formation of an ether or a carboxylic or carbamoyl ester linkage between the hydroxy and an alkyl group R—, an alkanoyl group RCO— or a substituted carbamoyl group RNHCO—. The alkyl group R may be cyclic (e.g., cyclohexyl) or acyclic (e.g., straight-chained or branched; and saturated or unsaturated). Alkyl group R may be a butyl, pentyl, hexyl, heptyl, octyl, nonyl, decyl, undecyl, dodecyl, tridecyl, tetradecyl, pentadecyl, hexadecyl, heptadecyl or octadecyl group, or the like.

In some embodiments, the C₂₂ hydrocarbon chain is conjugated to the dsRNA agent via a linker a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

In one embodiment, the one or more C₂₂ hydrocarbon chains is a C₂₂ acid, e.g., the C₂₂ acid is selected from the group consisting of docosanoic acid, 6-octyltetradecanoic acid, 10 hexylhexadecanoic acid, all-cis-7,10,13,16,19-docosapentaenoic acid, all-cis-4,7,10,13,16,19-docosahexaenoic acid, all-cis-13,16-docosadienoic acid, all-cis-7,10,13,16-docosatetraenoic acid, all-cis-4,7,10,13,16-docosapentaenoic acid, and cis-13-docosenoic acid.

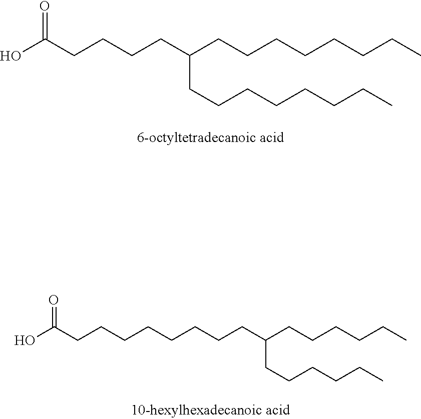 [SMILES: CCCCCCCCC(CCCCCCCC)CCCCC(=O)O ; CCCCCCC(CCCCCC)CCCCCCCCC(=O)O]

In one embodiment, the one or more C₂₂ hydrocarbon chains is a C₂₂ alcohol, e.g. the C₂₂ alcohol is selected from the group consisting of 1-docosanol, 6-octyltetradecan-1-ol, 10-hexylhexadecan-1-ol, cis-13-docosen-1-ol, docosan-9-ol, docosan-2-ol, docosan-10-ol, docosan-11-ol, and cis-4,7,10,13,16,19-docosahexanol.

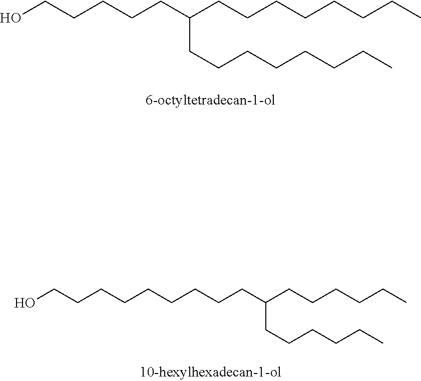 [SMILES: CCCCCCCCC(CCCCCO)CCCCCCCC ; CCCCCCC(CCCCCC)CCCCCCCCCO]

In one embodiment, the one or more C₂₂ hydrocarbon chains is not cis-4,7,10,13,16,19-docosahexanoic acid. In one embodiment, the one or more C₂₂ hydrocarbon chains is not cis-4,7,10,13,16,19-docosahexanol. In one embodiment, the one or more C₂₂ hydrocarbon chains is not cis-4,7,10,13,16,19-docosahexanoic acid and is not cis-4,7,10,13,16,19-docosahexanol.

In one embodiment, the one or more C₂₂ hydrocarbon chains is a C₂₂ amide, e.g., the C₂₂ amide is selected from the group consisting of (E)-Docos-4-enamide, (E)-Docos-5-enamide, (Z)-Docos-9-enamide, (E)-Docos-11-enamide, 12-Docosenamide, (Z)-Docos-13-enamide, (Z)—N-Hydroxy-13-docoseneamide, (E)-Docos-14-enamide, 6-cis-Docosenamide, 14-Docosenamide Docos-11-enamide, (4E,13E)-Docosa-4,13-dienamide, and (5E,13E)-Docosa-5,13-dienamide.

In certain embodiments, more than one C₂₂ hydrocarbon chains can be incorporated into the double-strand iRNA agent, particularly when the C₂₂ hydrocarbon chains has a low lipophilicity or hydrophobicity. In one embodiment, two or more C₂₂ hydrocarbon chains are incorporated into the same strand of the double-strand iRNA agent. In one embodiment, each strand of the double-strand iRNA agent has one or more C₂₂ hydrocarbon chains incorporated. In one embodiment, two or more C₂₂ hydrocarbon chains are incorporated into the same position (i.e., the same nucleobase, same sugar moiety, or same internucleosidic linkage) of the double-stranded iRNA agent. This can be achieved by, e.g., conjugating the two or more saturated or unsaturated C₂₂ hydrocarbon chains via a carrier, and/or conjugating the two or more C₂₂ hydrocarbon chains via a branched linker, and/or conjugating the two or more C₂₂ hydrocarbon chains via one or more linkers, with one or more linkers linking the C₂₂ hydrocarbon chains consecutively.

The one or more C₂₂ hydrocarbon chains may be conjugated to the iRNA agent via a direct attachment to the ribosugar of the iRNA agent. Alternatively, the one or more C₂₂ hydrocarbon chains may be conjugated to the double-strand iRNA agent via a linker or a carrier.

In certain embodiments, the one or more C₂₂ hydrocarbon chains may be conjugated to the iRNA agent via one or more linkers (tethers).

In one embodiment, the one or more C₂₂ hydrocarbon chains is conjugated to the dsRNA agent via a linker containing an ether, thioether, urea, carbonate, amine, amide, maleimide-thioether, disulfide, phosphodiester, sulfonamide linkage, a product of a click reaction (e.g., a triazole from the azide-alkyne cycloaddition), or carbamate.

### A. Linkers/Tethers

Linkers/Tethers are connected to the one or more C₂₂ hydrocarbon chains at a “tethering attachment point (TAP).” Linkers/Tethers may include any C₁-C₁₀₀ carbon-containing moiety, (e.g. C₁-C₇₅, C₁-C₅₀, C₁-C₂₀, C₁-C₁₀; C₁, C₂, C₃, C₄, C₅, C₆, C₇, C₈, C₉, or C₁₀), and may have at least one nitrogen atom. In certain embodiments, the nitrogen atom forms part of a terminal amino or amido (NHC(O)—) group on the linker/tether, which may serve as a connection point for the lipophilic moiety. Non-limited examples of linkers/tethers (underlined) include TAP-(CH₂)₂NH—; TAP-C(O)(CH₂)ₙNH—; TAP-NR″″(CH₂)ₙNH—, TAP-C(O)—(CH₂)ₙ—C(O)—; TAP-C(O)—(CH₂)ₙ—C(O)O—; TAP-C(O)—O—; TAP-C(O)—(CH₂)ₙ—NH—C(O)—; TAP-C(O)—(CH₂)ₙ—; TAP-C(O)—NH—; TAP-C(O)—; TAP-(CH₂)ₙ—C(O)—; TAP-(CH₂)ₙ—C(O)O—; TAP-(CH₂)ₙ—, or TAP-(CH₂)ₙ—NH—C(O)—; in which n is 1-20 (e.g., 1, 2, 3, 4, 5, 6, 7, 8, 9, 10, 11, 12, 13, 14, 15, 16, 17, 18, 19, or 20) and R″″ is C₁-C₆ alkyl. Preferably, n is 5, 6, or 11. In other embodiments, the nitrogen may form part of a terminal oxyamino group, e.g., —ONH₂, or hydrazino group, —NHNH₂. The linker/tether may optionally be substituted, e.g., with hydroxy, alkoxy, perhaloalkyl, and/or optionally inserted with one or more additional heteroatoms, e.g., N, O, or S. Preferred tethered ligands may include, e.g., TAP-(CH₂)ₙNH(LIGAND); TAP-C(O)(CH₂)ₙNH(LIGAND); TAP-NR″″(CH₂)ₙNH(LIGAND); TAP-(CH₂)ₙNH(LIGAND); TAP-C(O)(CH₂)ₙNH(LIGAND); TAP-NR″″(CH₂)ₙONH(LIGAND); TAP-(CH₂)ₙNHNH₂(LIGAND), TAP-C(O)(CH₂)ₙNHNH₂(LIGAND); TAP-NR″″(CH₂)ₙNHNH₂(LIGAND); TAP-C(O)—(CH₂)ₙ—C(O)(LIGAND); TAP-C(O)—(CH₂)ₙ—C(O)O(LIGAND); TAP-C(O)—O(LIGAND); TAP-C(O)—(CH₂)ₙ—NH—C(O)(LIGAND); TAP-C(O)—(CH₂)ₙ(LIGAND); TAP-C(O)—NH(LIGAND); TAP-C(O)(LIGAND); TAP-(CH₂)ₙ—C(O) (LIGAND); TAP-(CH₂)ₙ—C(O)O(LIGAND); TAP-(CH₂)ₙ(LIGAND); or TAP-(CH₂)ₙ—NH—C(O)(LIGAND). In some embodiments, amino terminated linkers/tethers (e.g., NH₂, ONH₂, NH₂NH₂) can form an imino bond (i.e., C═N) with the ligand. In some embodiments, amino terminated linkers/tethers (e.g., NH₂, ONH₂, NH₂NH₂) can acylated, e.g., with C(O)CF₃.

In some embodiments, the linker/tether can terminate with a mercapto group (i.e., SH) or an olefin (e.g., CH═CH₂). For example, the tether can be TAP-(CH₂)ₙ—SH, TAP-C(O)(CH₂)ₙSH, TAP-(CH₂)ₙ—(CH═CH₂), or TAP-C(O)(CH₂)ₙ(CH═CH₂), in which n can be as described elsewhere. The tether may optionally be substituted, e.g., with hydroxy, alkoxy, perhaloalkyl, and/or optionally inserted with one or more additional heteroatoms, e.g., N, O, or S. The double bond can be cis or trans or E or Z.

In other embodiments, the linker/tether may include an electrophilic moiety, preferably at the terminal position of the linker/tether. Exemplary electrophilic moieties include, e.g., an aldehyde, alkyl halide, mesylate, tosylate, nosylate, or brosylate, or an activated carboxylic acid ester, e.g. an NHS ester, or a pentafluorophenyl ester. Preferred linkers/tethers (underlined) include TAP-(CH₂)ₙCHO; TAP-C(O)(CH₂)ₙCHO; or TAP-NR″″(CH₂)ₙCHO, in which n is 1-6 and R″″ is C₁-C₆ alkyl; or TAP-(CH₂)ₙC(O)ONHS; TAP-C(O)(CH₂)ₙC(O)ONHS; or TAP-NR″″(CH₂)ₙC(O)ONHS, in which n is 1-6 and R″″ is C₁-C₆ alkyl; TAP-(CH₂)ₙC(O)OC₆F₅; TAP-C(O)(CH₂)ₙC(O) OC₆F₅; or TAP-NR″″(CH₂)ₙC(O) OC₆F₅, in which n is 1-11 and R″″ is C₁-C₆ alkyl; or —(CH₂)ₙCH₂LG; TAP-C(O)(CH₂)ₙCH₂LG; or TAP-NR″″(CH₂)ₙCH₂LG, in which n can be as described elsewhere and R″″ is C₁-C₆ alkyl (LG can be a leaving group, e.g., halide, mesylate, tosylate, nosylate, brosylate). Tethering can be carried out by coupling a nucleophilic group of a ligand, e.g., a thiol or amino group with an electrophilic group on the tether.

In other embodiments, it can be desirable for the monomer to include a phthalimido group (K) at the terminal position of the linker/tether

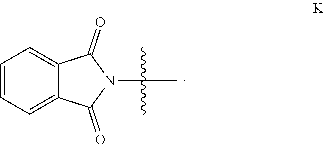 [SMILES: *N1C(=O)c2ccccc2C1=O]

In other embodiments, other protected amino groups can be at the terminal position of the linker/tether, e.g., alloc, monomethoxy trityl (MMT), trifluoroacetyl, Fmoc, or aryl sulfonyl (e.g., the aryl portion can be ortho-nitrophenyl or ortho, para-dinitrophenyl).

Any of the linkers/tethers described herein may further include one or more additional linking groups, e.g., —O—(CH₂)ₙ—, —(CH₂)ₙ—SS—, —(CH₂)ₙ—, or —(CH═CH)—.

### B. Cleavable Linkers/Tethers

In some embodiments, at least one of the linkers/tethers can be a redox cleavable linker, an acid cleavable linker, an esterase cleavable linker, a phosphatase cleavable linker, or a peptidase cleavable linker.

In one embodiment, at least one of the linkers/tethers can be a reductively cleavable linker (e.g., a disulfide group).

In one embodiment, at least one of the linkers/tethers can be an acid cleavable linker (e.g., a hydrazone group, an ester group, an acetal group, or a ketal group).

In one embodiment, at least one of the linkers/tethers can be an esterase cleavable linker (e.g., an ester group).

In one embodiment, at least one of the linkers/tethers can be a phosphatase cleavable linker (e.g., a phosphate group).

In one embodiment, at least one of the linkers/tethers can be a peptidase cleavable linker (e.g., a peptide bond).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some tethers will have a linkage group that is cleaved at a preferred pH, thereby releasing the iRNA agent from a ligand (e.g., a targeting or cell-permeable ligand, such as cholesterol) inside the cell, or into the desired compartment of the cell.

A chemical junction (e.g., a linking group) that links a ligand to an iRNA agent can include a disulfide bond. When the iRNA agent/ligand complex is taken up into the cell by endocytosis, the acidic environment of the endosome will cause the disulfide bond to be cleaved, thereby releasing the iRNA agent from the ligand (Quintana et al., Pharm Res. 19:1310-1316, 2002; Patri et al., Curr. Opin. Curr. Biol. 6:466-471, 2002). The ligand can be a targeting ligand or a second therapeutic agent that may complement the therapeutic effects of the iRNA agent.

A tether can include a linking group that is cleavable by a particular enzyme. The type of linking group incorporated into a tether can depend on the cell to be targeted by the iRNA agent. For example, an iRNA agent that targets an mRNA in liver cells can be conjugated to a tether that includes an ester group. Liver cells are rich in esterases, and therefore the tether will be cleaved more efficiently in liver cells than in cell types that are not esterase-rich. Cleavage of the tether releases the iRNA agent from a ligand that is attached to the distal end of the tether, thereby potentially enhancing silencing activity of the iRNA agent. Other cell-types rich in esterases include cells of the lung, renal cortex, and testis.

Tethers that contain peptide bonds can be conjugated to iRNA agents target to cell types rich in peptidases, such as liver cells and synoviocytes. For example, an iRNA agent targeted to synoviocytes, such as for the treatment of an inflammatory disease (e.g., rheumatoid arthritis), can be conjugated to a tether containing a peptide bond.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue, e.g., tissue the iRNA agent would be exposed to when administered to a subject. Thus one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It may be useful to make initial evaluations in cell-free or culture conditions and to confirm by further evaluations in whole animals. In preferred embodiments, useful candidate compounds are cleaved at least 2, 4, 10 or 100 times faster in the cell (or under in vitro conditions selected to mimic intracellular conditions) as compared to blood or serum (or under in vitro conditions selected to mimic extracellular conditions).

### C. Redox Cleavable Linking Groups

One class of cleavable linking groups are redox cleavable linking groups that are cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTI), or other reducing agent using reagents know in the art, which mimic the rate of cleavage which would be observed in a cell, e.g., a target cell. The candidates can also be evaluated under conditions which are selected to mimic blood or serum conditions. In a preferred embodiment, candidate compounds are cleaved by at most 10% in the blood. In preferred embodiments, useful candidate compounds are degraded at least 2, 4, 10 or 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### D. Phosphate-Based Cleavable Linking Groups

Phosphate-based linking groups are cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, —O—P(S)(H)—S—. A preferred embodiment is —O—P(O)(OH)—O—. These candidates can be evaluated using methods analogous to those described above.

### E. Acid Cleavable Linking Groups

Acid cleavable linking groups are linking groups that are cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, ketals, acetals, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### F. Ester-Based Linking Groups

Ester-based linking groups are cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

### G. Peptide-Based Cleaving Groups

Peptide-based linking groups are cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide cleavable linking groups have the general formula —NHCHR¹C(O)NHCHR²C(O)—, where R¹ and R² are the R groups of the two adjacent amino acids. These candidates can be evaluated using methods analogous to those described above.

### H. Biocleavable Linkers/Tethers

The linkers can also includes biocleavable linkers that are nucleotide and non-nucleotide linkers or combinations thereof that connect two parts of a molecule, for example, one or both strands of two individual siRNA molecules to generate a bis(siRNA). In some embodiments, mere electrostatic or stacking interaction between two individual siRNAs can represent a linker. The non-nucleotide linkers include tethers or linkers derived from monosaccharides, disaccharides, oligosaccharides, and derivatives thereof, aliphatic, alicyclic, hetercyclic, and combinations thereof.

In some embodiments, at least one of the linkers (tethers) is a bio-cleavable linker selected from the group consisting of DNA, RNA, disulfide, amide, functionalized monosaccharides or oligosaccharides of galactosamine, glucosamine, glucose, galactose, and mannose, and combinations thereof.

In one embodiment, the bio-cleavable carbohydrate linker may have 1 to 10 saccharide units, which have at least one anomeric linkage capable of connecting two siRNA units. When two or more saccharides are present, these units can be linked via 1-3, 1-4, or 1-6 sugar linkages, or via alkyl chains.

Exemplary bio-cleavable linkers include:

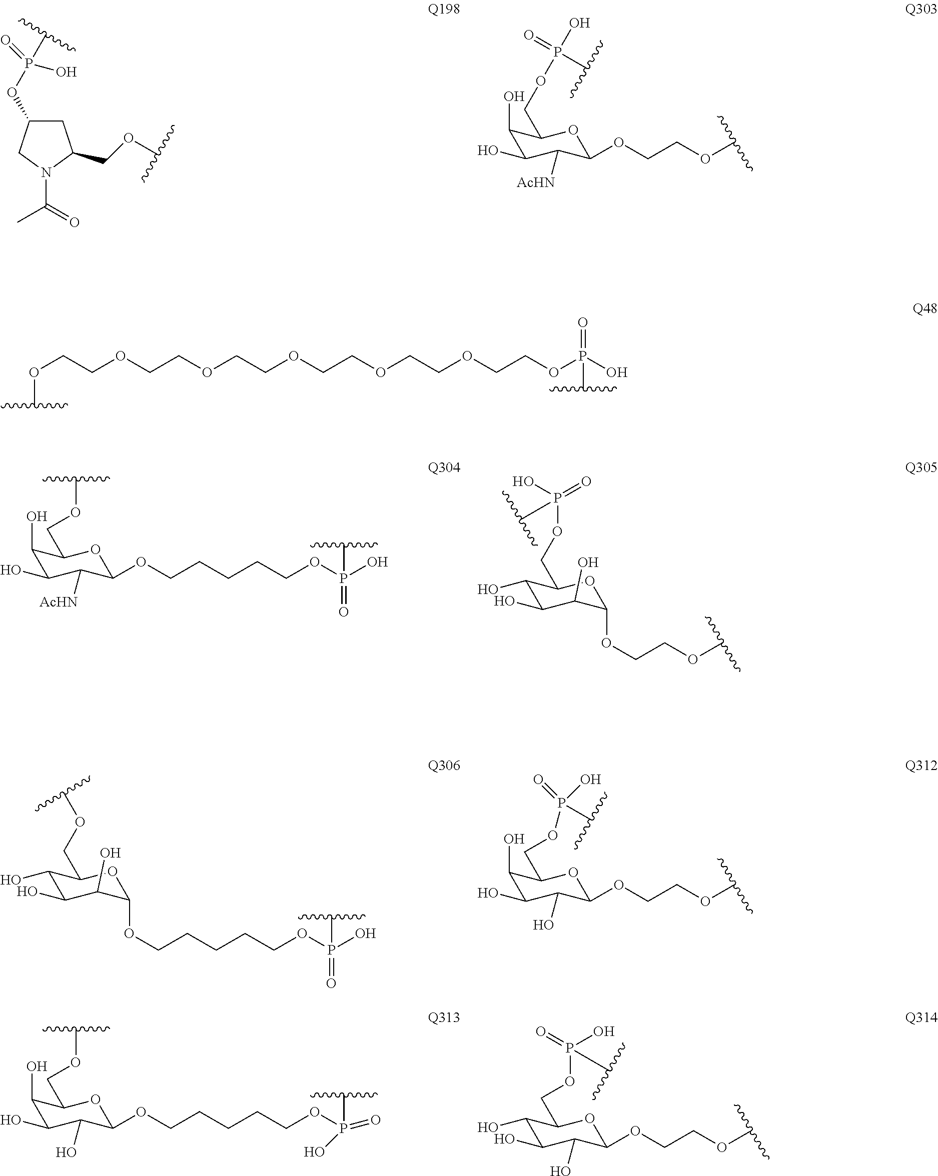

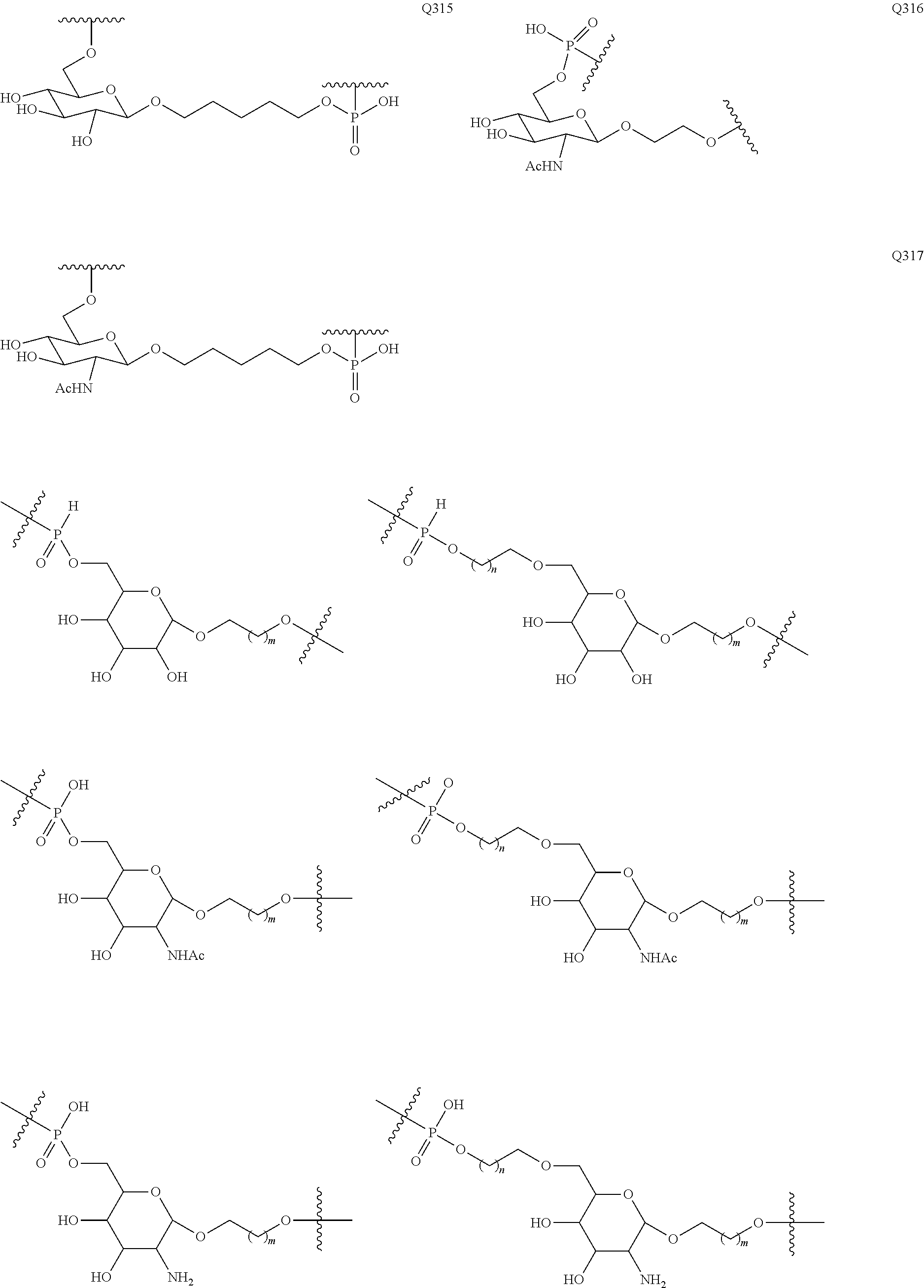

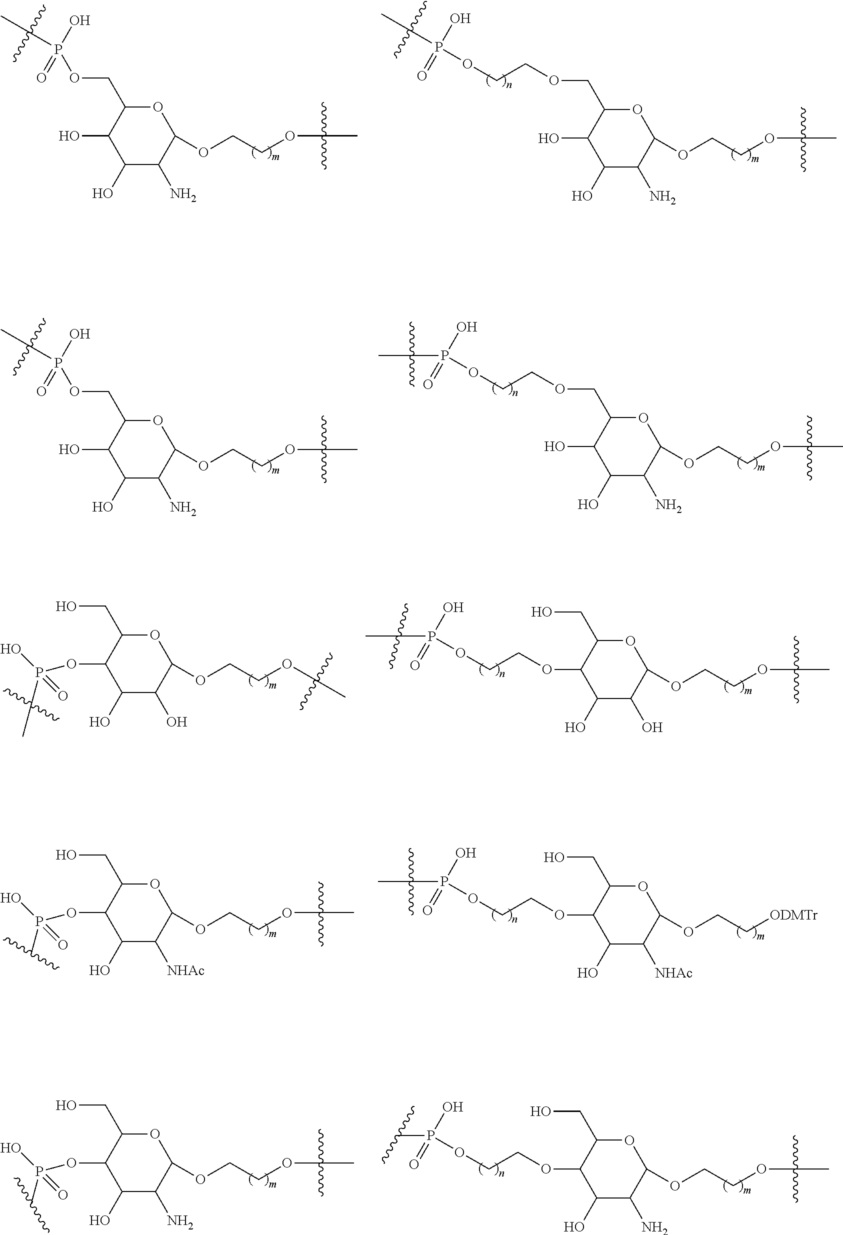

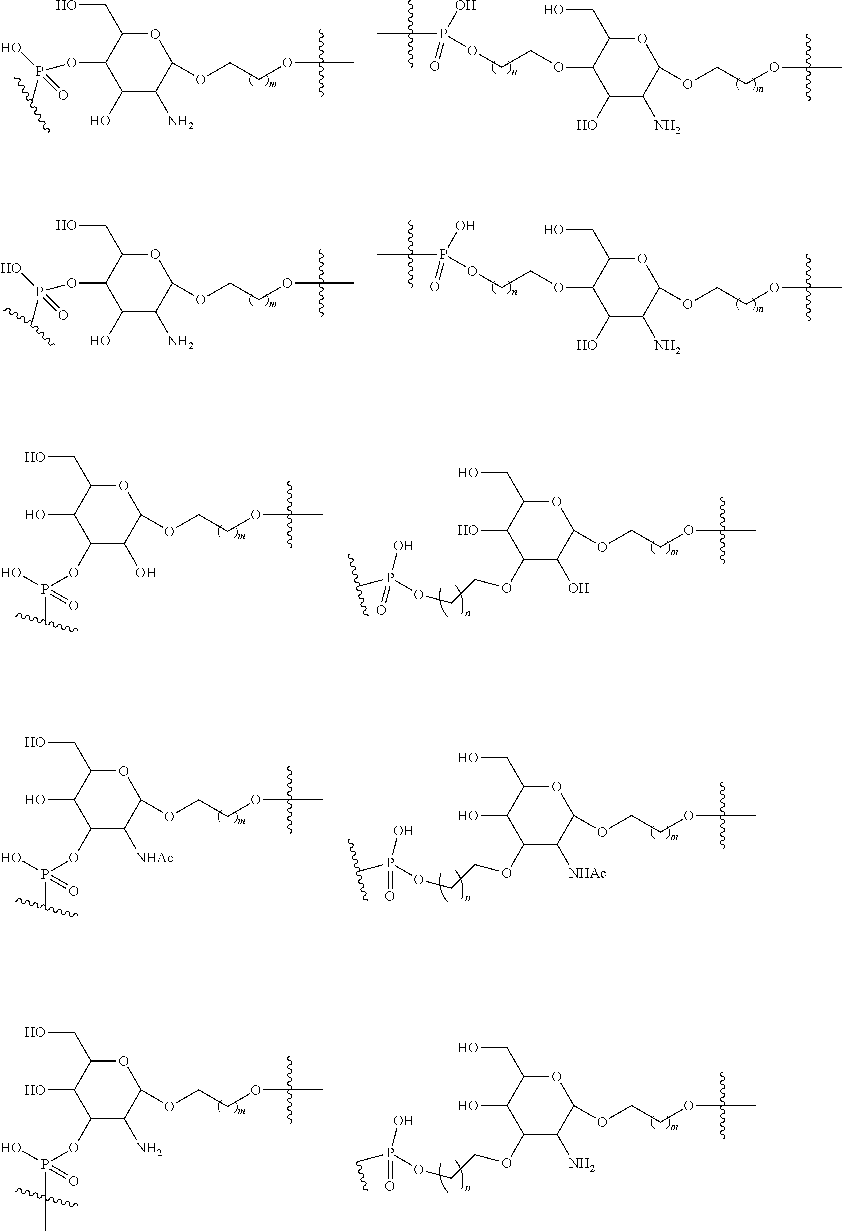

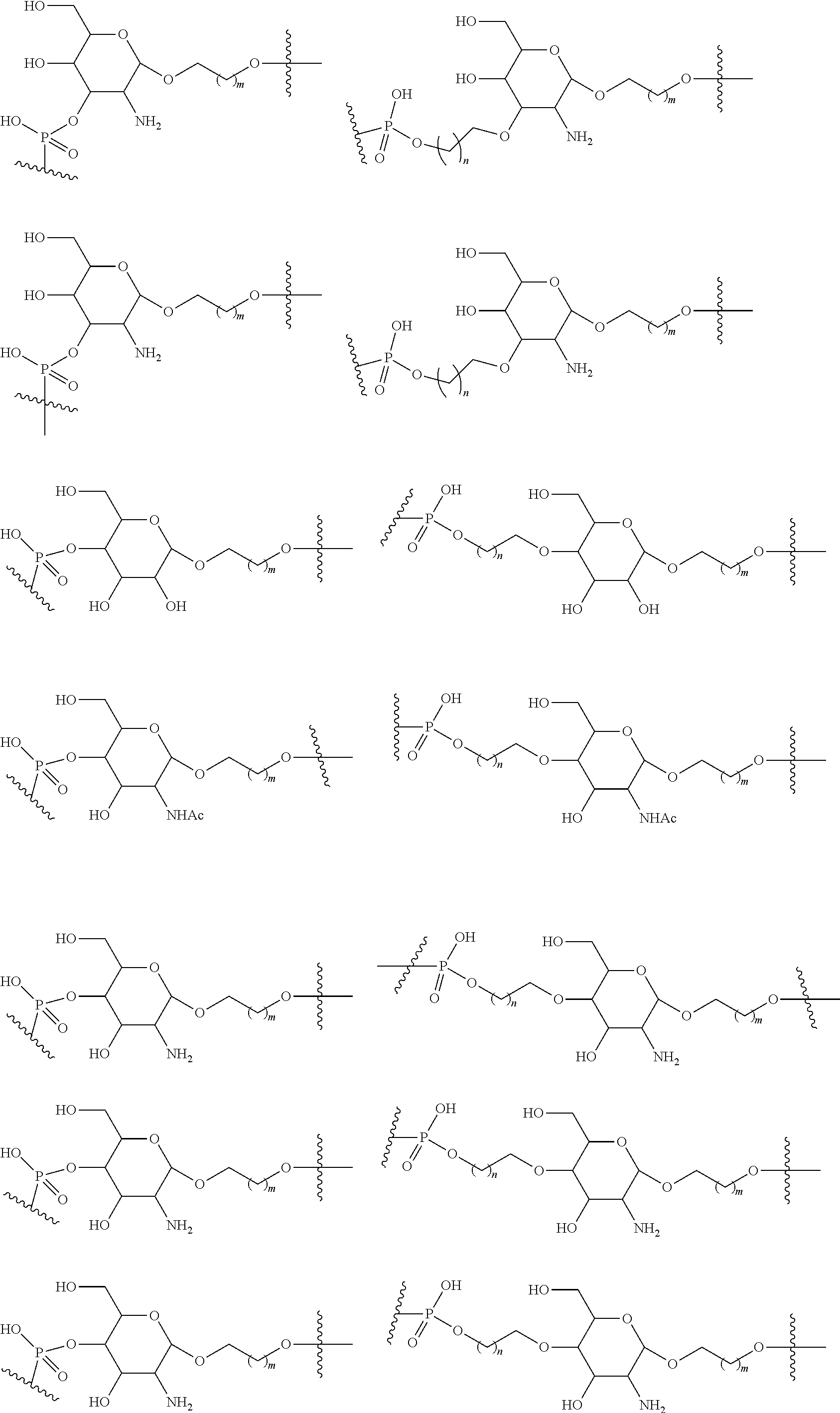

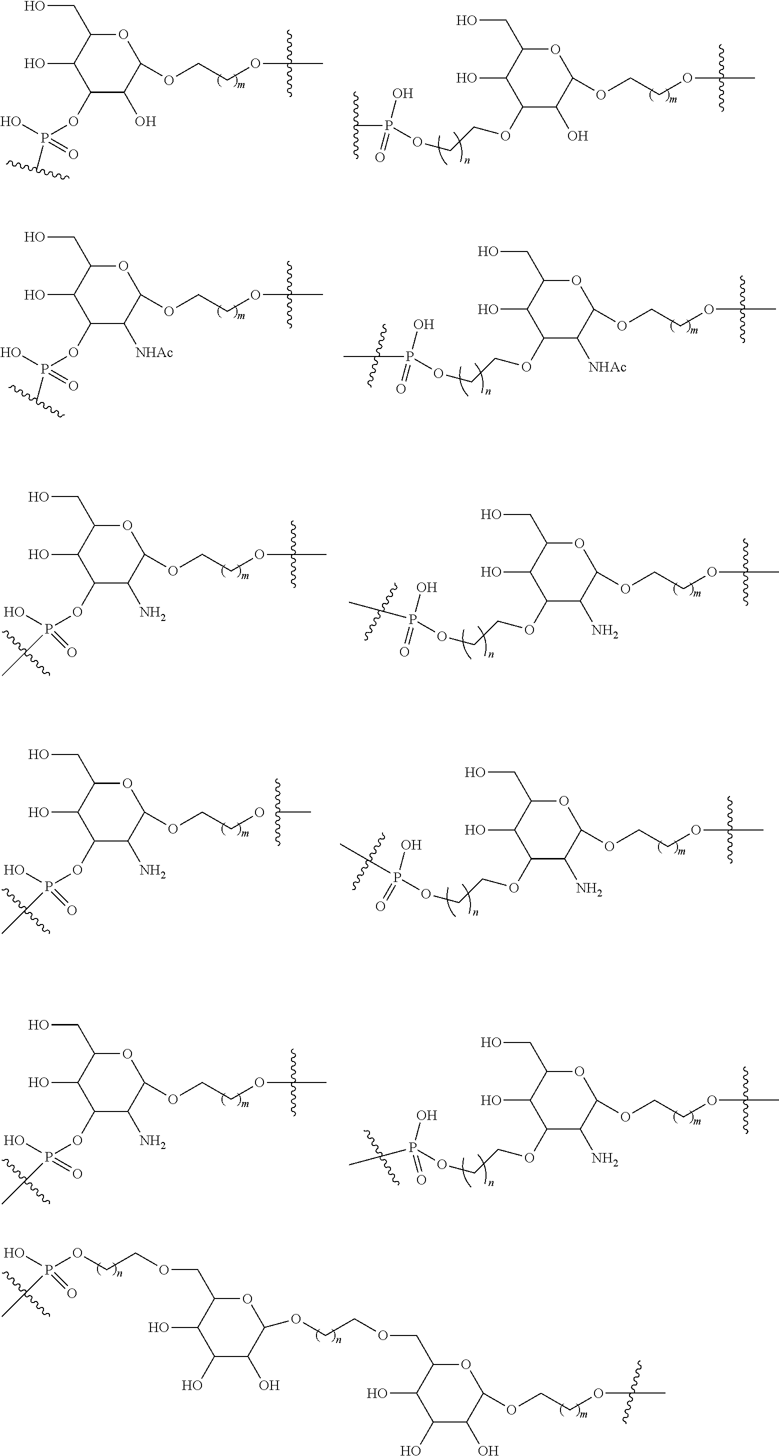

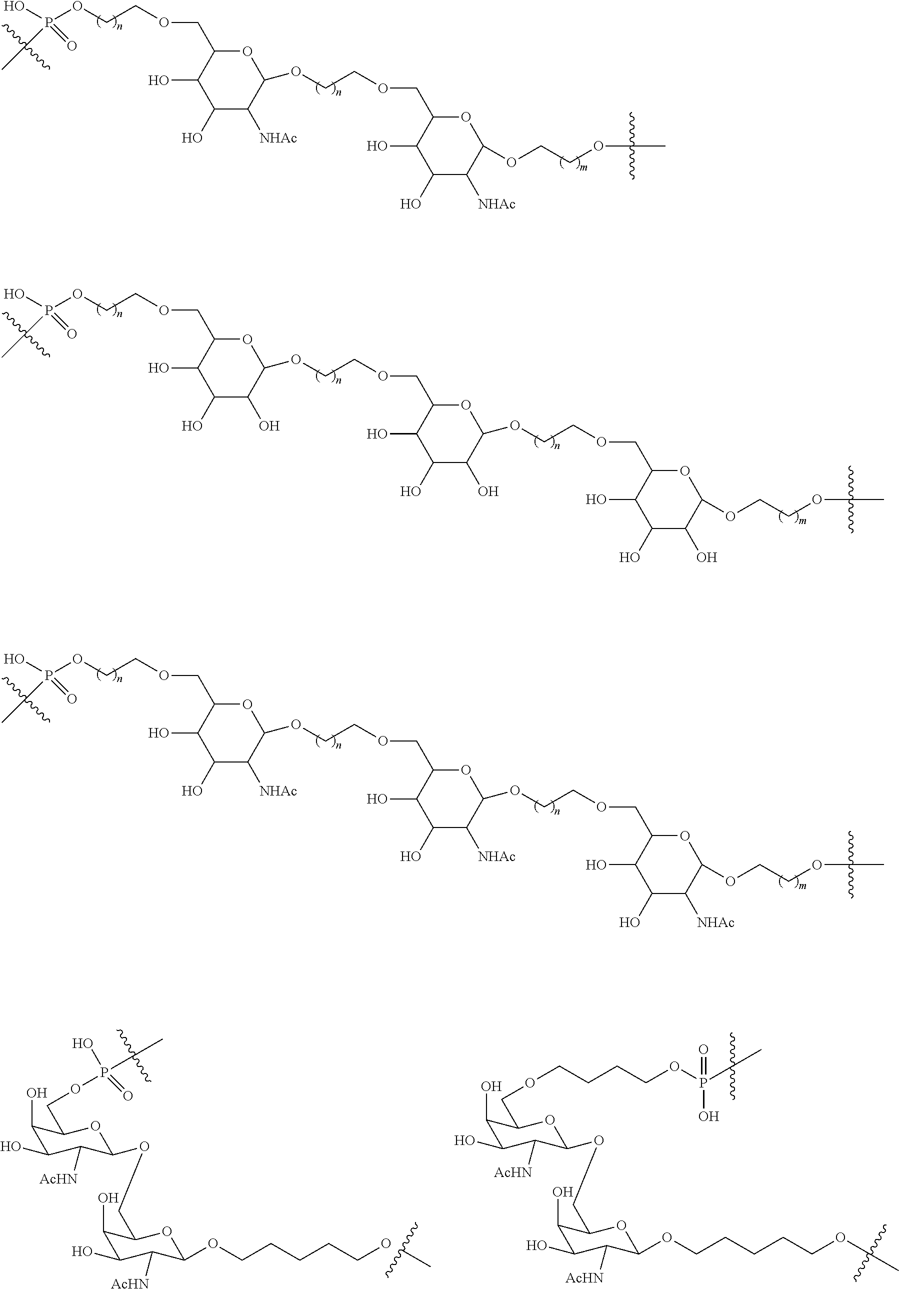

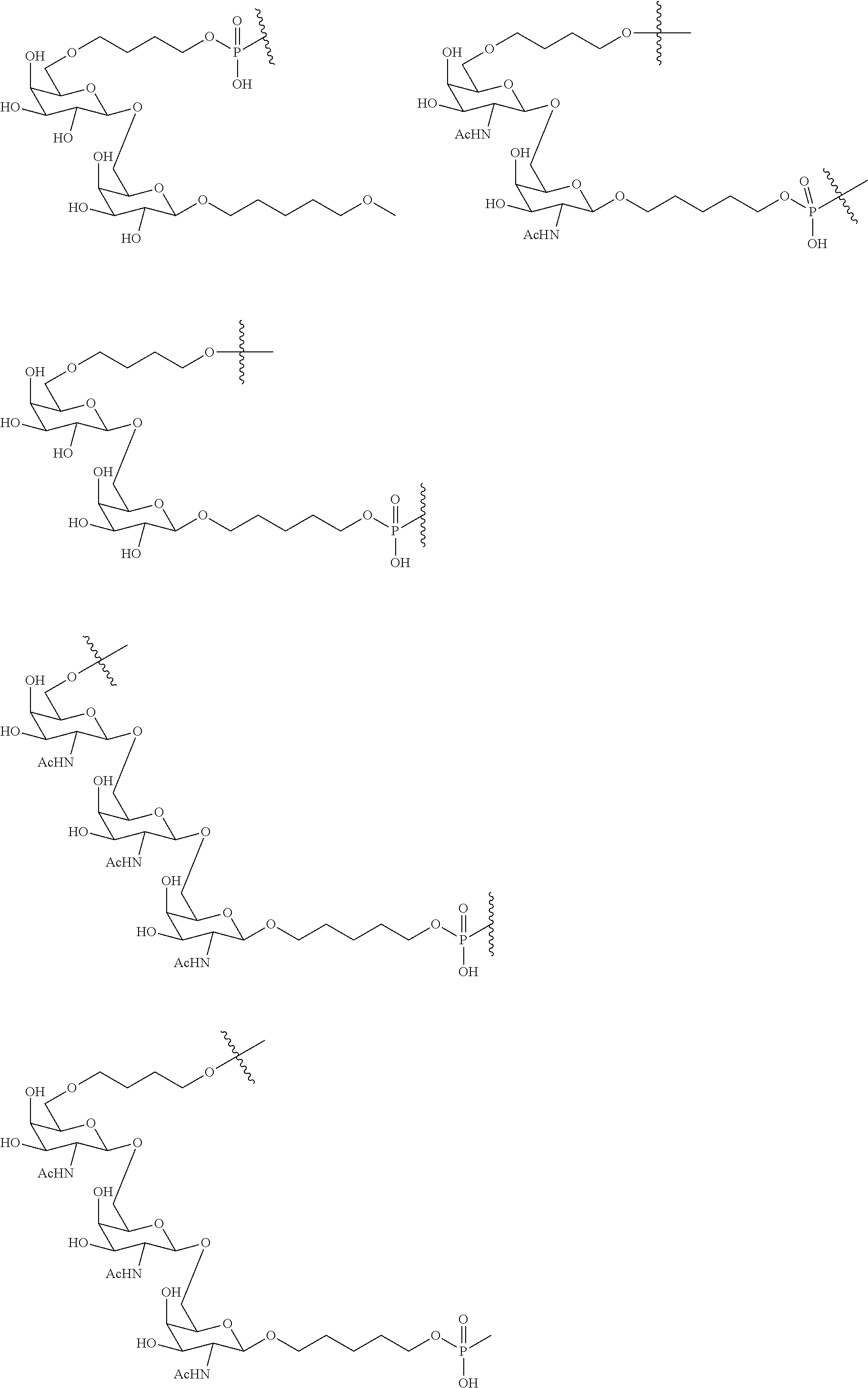 [SMILES: *P(=O)(O)OCCCCOCC1OC(OCC2OC(OCCCCCOC)C(O)C(O)C2O)C(O)C(O)C1O ; *OCCCCOCC1OC(OCC2OC(OCCCCCOP(*)(=O)O)C(NC(C)=O)C(O)C2O)C(NC(C)=O)C(O)C1O ; *OCCCCOCC1OC(OCC2OC(OCCCCCOP(*)(=O)O)C(O)C(O)C2O)C(O)C(O)C1O ; *OCC1OC(OCC2OC(OCC3OC(OCCCCCOP(*)(=O)O)C(NC(C)=O)C(O)C3O)C(NC(C)=O)C(O)C2O)C(NC(C)=O)C(O)C1O ; *OCCCCOCC1OC(OCC2OC(OCC3OC(OCCCCCOP(C)(=O)O)C(NC(C)=O)C(O)C3O)C(NC(C)=O)C(O)C2O)C(NC(C)=O)C(O)C1O]

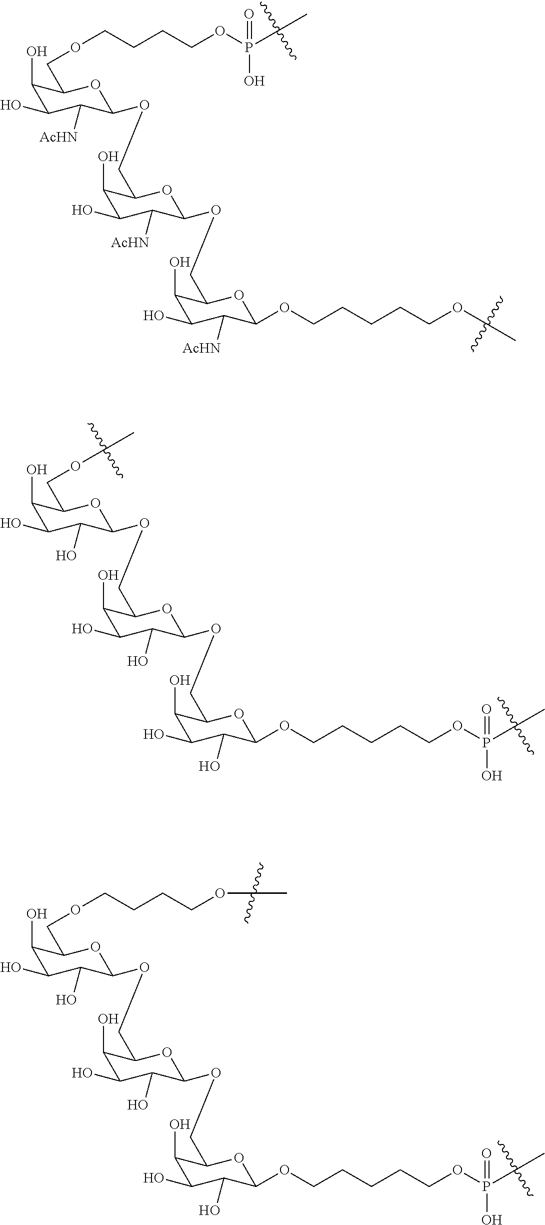 [SMILES: *OCCCCCOC1OC(COC2OC(COC3OC(COCCCCOP(*)(=O)O)C(O)C(O)C3NC(C)=O)C(O)C(O)C2NC(C)=O)C(O)C(O)C1NC(C)=O ; *OCC1OC(OCC2OC(OCC3OC(OCCCCCOP(*)(=O)O)C(O)C(O)C3O)C(O)C(O)C2O)C(O)C(O)C1O ; *OCCCCOCC1OC(OCC2OC(OCC3OC(OCCCCCOP(*)(=O)O)C(O)C(O)C3O)C(O)C(O)C2O)C(O)C(O)C1O]

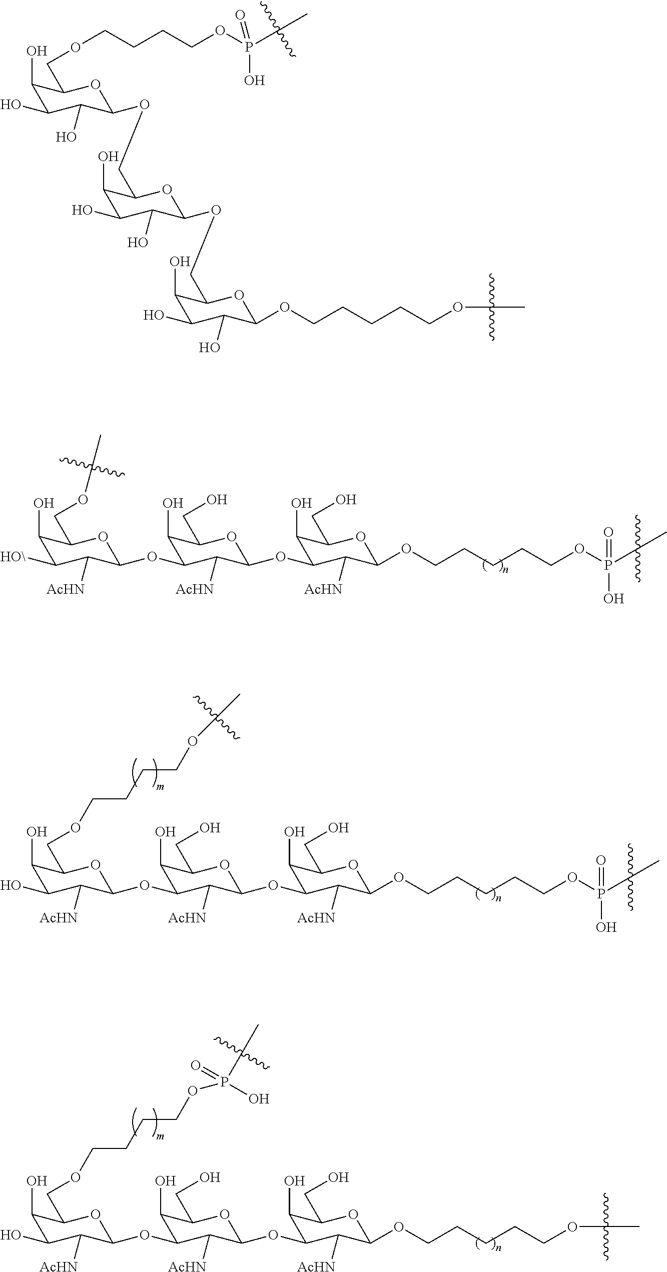

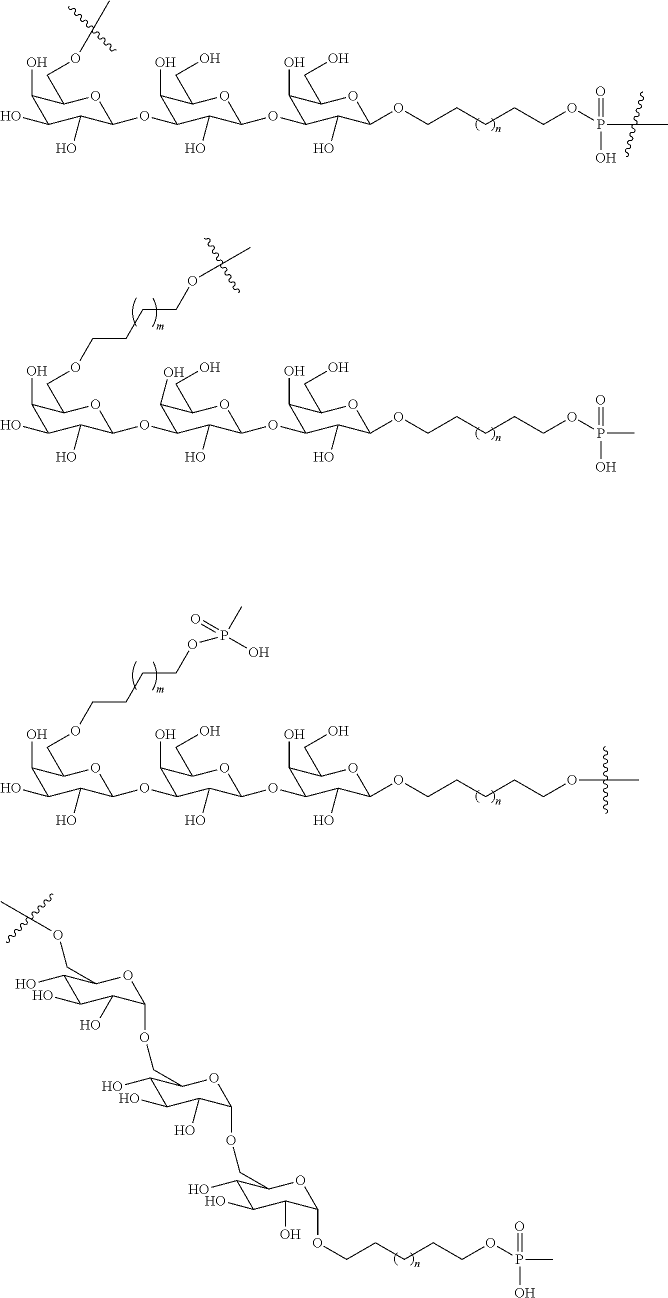

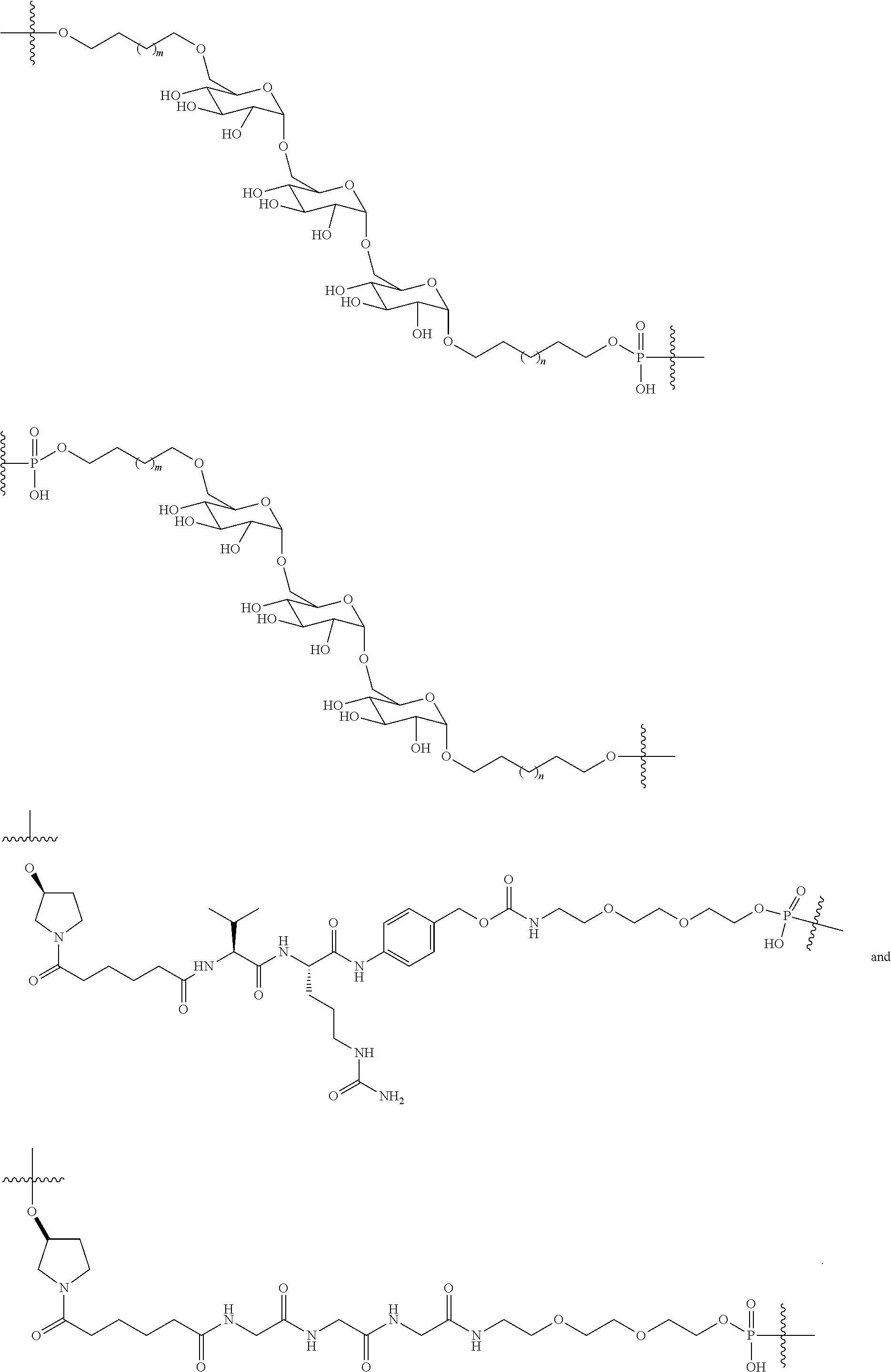

More discussion about the biocleavable linkers may be found in PCT application No. PCT/US18/14213, entitled “Endosomal Cleavable Linkers,” filed on Jan. 18, 2018, the entire contents of which are incorporated herein by reference.

### I. Carriers

In certain embodiments, the one or more C₂₂ hydrocarbon chains is conjugated to the iRNA agent via a carrier that replaces one or more nucleotide(s).

The carrier can be a cyclic group or an acyclic group. In one embodiment, the cyclic group is selected from the group consisting of pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin. In one embodiment, the acyclic group is a moiety based on a serinol backbone or a diethanolamine backbone.

In some embodiments, the carrier replaces one or more nucleotide(s) in the internal position(s) of the dsRNA agent.

In other embodiments, the carrier replaces the nucleotides at the terminal end of the sense strand or antisense strand. In one embodiment, the carrier replaces the terminal nucleotide on the 3′ end of the sense strand, thereby functioning as an end cap protecting the 3′ end of the sense strand. In one embodiment, the carrier is a cyclic group having an amine, for instance, the carrier may be pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, or decalinyl.

A ribonucleotide subunit in which the ribose sugar of the subunit has been so replaced is referred to herein as a ribose replacement modification subunit (RRMS). The carrier can be a cyclic or acyclic moiety and include two “backbone attachment points” (e.g., hydroxyl groups) and a ligand (e.g., the lipophilic moiety). The one or more C₂₂ hydrocarbon chains can be directly attached to the carrier or indirectly attached to the carrier by an intervening linker/tether, as described above.

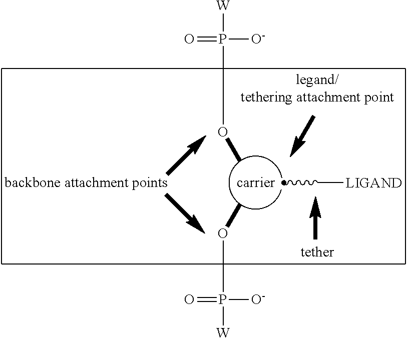

The ligand-conjugated monomer subunit may be the 5′ or 3′ terminal subunit of the iRNA molecule, i.e., one of the two “W” groups may be a hydroxyl group, and the other “W” group may be a chain of two or more unmodified or modified ribonucleotides. Alternatively, the ligand-conjugated monomer subunit may occupy an internal position, and both “W” groups may be one or more unmodified or modified ribonucleotides. More than one ligand-conjugated monomer subunit may be present in an iRNA agent.

a. Sugar Replacement-Based Monomers. e.g., Ligand-Conjugated Monomers (Cyclic)

Cyclic sugar replacement-based monomers, e.g., sugar replacement-based ligand-conjugated monomers, are also referred to herein as RRMS monomer compounds. The carriers may have the general formula (LCM-2) provided below (in that structure preferred backbone attachment points can be chosen from R¹ or R²; R³ or R⁴; or R⁹ and R¹⁰ if Y is CR⁹R¹⁰ (two positions are chosen to give two backbone attachment points, e.g., R¹ and R⁴, or R⁴ and R⁹)). Preferred tethering attachment points include R⁷; R⁵ or R⁶ when X is CH₂. The carriers are described below as an entity, which can be incorporated into a strand. Thus, it is understood that the structures also encompass the situations wherein one (in the case of a terminal position) or two (in the case of an internal position) of the attachment points, e.g., R¹ or R²; R³ or R⁴; or R⁹ or R¹⁰ (when Y is CR⁹R¹⁰), is connected to the phosphate, or modified phosphate, e.g., sulfur containing, backbone. E.g., one of the above-named R groups can be —CH₂—, wherein one bond is connected to the carrier and one to a backbone atom, e.g., a linking oxygen or a central phosphorus atom.

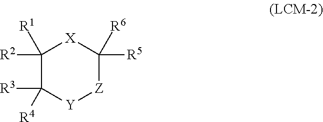

wherein:
X is N(CO)R⁷, NR₇ or CH₂;
Y is NR⁸, O, S, CR⁹R¹⁰;
Z is CR¹¹R¹² or absent;
Each of R¹, R², R³, R⁴, R⁹, and R¹⁰ is, independently, H, OR¹, or (CH₂)₆OR^{b}, provided that at least two of R¹, R², R³, R⁴, R⁹, and R¹⁰ are OR^{a} and/or (CH₂)ₙOR^{b};
Each of R⁵, R⁶, R¹¹, and R¹² is, independently, a ligand, H, C₁-C₆ alkyl optionally substituted with 1-3 R¹³, or C(O)NHR⁷; or R⁵ and R¹¹ together are C₃-C₈ cycloalkyl optionally substituted with R¹⁴;
R⁷ can be a ligand, e.g., R⁷ can be R⁴, or R⁷ can be a ligand tethered indirectly to the carrier, e.g., through a tethering moiety, e.g., C₁-C₂₀ alkyl substituted with NR^{c}R^{d}; or C₁-C₂₀ alkyl substituted with NHC(O)R^{d};
R⁸ is H or C₁-C₆ alkyl;
R¹³ is hydroxy, C₁-C₄ alkoxy, or halo;
R¹⁴ is NR^{c}R⁷;
R¹⁵ is C₁-C₆ alkyl optionally substituted with cyano, or C₂-C₆ alkenyl;
R¹⁶ is C₁-C₁₀ alkyl;
R¹⁷ is a liquid or solid phase support reagent;
L is —C(O)(CH₂)_{q}C(O)—, or —C(O)(CH₂)_{q}S—;
R^{a} is a protecting group, e.g., CAr₃; (e.g., a dimethoxytrityl group) or Si(X^{5′})(X^{5″})(X^{5′″}) in which (X^{5′}), (X^{5″}), and (X^{5′″}) are as described elsewhere.
R^{b} is P(O)(O⁻)H, P(OR¹⁵)N(R¹⁶)₂ or L-R¹⁷;
R^{c} is H or C₁-C₆ alkyl;
R^{d} is H or a ligand;
Each Ar is, independently, C₆-C₁₀ aryl optionally substituted with C₁-C₄ alkoxy;
n is 1-4; and q is 0-4.

Exemplary carriers include those in which, e.g., X is N(CO)R⁷ or NR₇, Y is CR⁹R¹⁰, and Z is absent; or X is N(CO)R⁷ or NR₇, Y is CR⁹R¹⁰, and Z is CR¹¹R¹²; or X is N(CO)R⁷ or NR₇, Y is NR₈, and Z is CR¹¹R¹²; or X is N(CO)R⁷ or NR⁷, Y is O, and Z is CR¹¹R¹²; or X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

In certain embodiments, the carrier may be based on the pyrroline ring system or the 4-hydroxyproline ring system, e.g., X is N(CO)R⁷ or NR⁷, Y is CR⁹R¹⁰, and Z is absent (D).

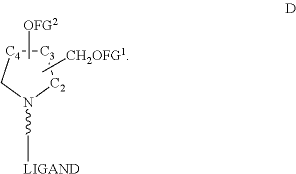

OFG¹ is preferably attached to a primary carbon, e.g., an exocyclic alkylene group, e.g., a methylene group, connected to one of the carbons in the five-membered ring (—CH₂OFG¹ in D). OFG² is preferably attached directly to one of the carbons in the five-membered ring (—OFG² in D). For the pyrroline-based carriers, —CH₂OFG¹ may be attached to C-2 and OFG² may be attached to C-3; or -CH₂OFG¹ may be attached to C-3 and OFG² may be attached to C-4. In certain embodiments, CH₂OFG¹ and OFG² may be geminally substituted to one of the above-referenced carbons. For the 3-hydroxyproline-based carriers, —CH₂OFG¹ may be attached to C-2 and OFG² may be attached to C-4. The pyrroline- and 4-hydroxyproline-based monomers may therefore contain linkages (e.g., carbon-carbon bonds) wherein bond rotation is restricted about that particular linkage, e.g. restriction resulting from the presence of a ring. Thus, CH₂OFG¹ and OFG² may be cis or trans with respect to one another in any of the pairings delineated above Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen. Preferred examples of carrier D include the following:

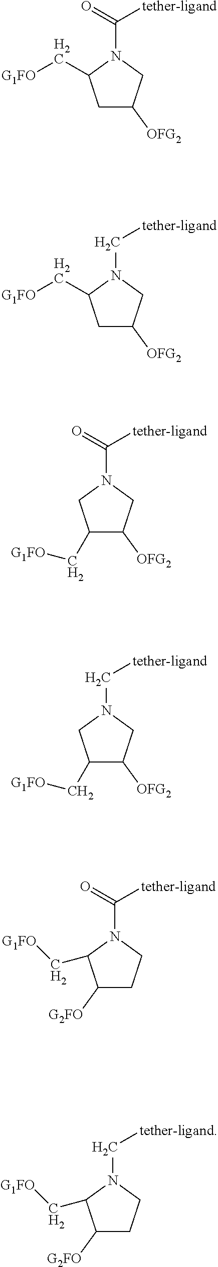

In certain embodiments, the carrier may be based on the piperdine ring system (E), e.g., X is N(CO)R⁷ or NR⁷, Y is CR⁹R¹⁰, and Z is CR¹¹R¹².

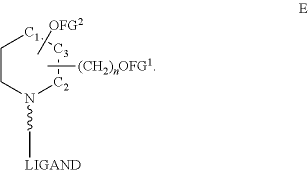

OFG¹ is preferably attached to a primary carbon, e.g., an exocyclic alkylene group, e.g., a methylene group (n=1) or ethylene group (n=2), connected to one of the carbons in the six-membered ring [—(CH₂)ₙOFG¹ in E]. OFG² is preferably attached directly to one of the carbons in the six-membered ring (—OFG² in E). —(CH₂)ₙOFG¹ and OFG² may be disposed in a geminal manner on the ring, i.e., both groups may be attached to the same carbon, e.g., at C-2, C-3, or C-4. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3. The piperdine-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

In certain embodiments, the carrier may be based on the piperazine ring system (F), e.g., X is N(CO)R⁷ or NR⁷, Y is NR⁸, and Z is CR¹¹R¹², or the morpholine ring system (G), e.g., X is N(CO)R⁷ or NR⁷, Y is O, and Z is CR¹¹R¹².

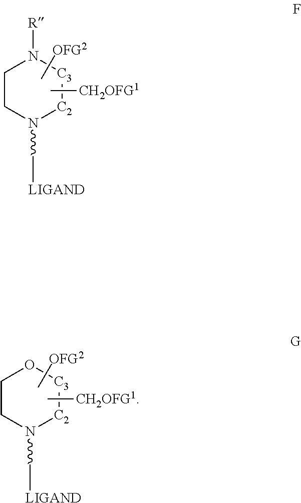

OFG¹ is preferably attached to a primary carbon, e.g., an exocyclic alkylene group, e.g., a methylene group, connected to one of the carbons in the six-membered ring (—CH₂OFG¹ in F or G). OFG² is preferably attached directly to one of the carbons in the six-membered rings (—OFG² in F or G). For both F and G, —CH₂OFG¹ may be attached to C-2 and OFG² may be attached to C-3; or vice versa. In certain embodiments, CH₂OFG¹ and OFG² may be geminally substituted to one of the above-referenced carbons. The piperazine- and morpholine-based monomers may therefore contain linkages (e.g., carbon-carbon bonds) wherein bond rotation is restricted about that particular linkage, e.g. restriction resulting from the presence of a ring. Thus, CH OFG¹ and OFG² may be cis or trans with respect to one another in any of the pairings delineated above. Accordingly, all cis trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). R′″ can be, e.g., C₁-C₆ alkyl, preferably CH₃. The tethering attachment point is preferably nitrogen in both F and G.

In certain embodiments, the carrier may be based on the decalin ring system, e.g., X is CH₂; Y is CR⁹R¹⁰; Z is CR¹¹R¹², and R⁵ and R¹¹ together form C₆ cycloalkyl (H, z=2), or the indane ring system, e.g., X is CH₂; Y is CR⁹R¹⁰; Z is CR¹¹R¹², and R⁵ and R¹¹ together form C₅ cycloalkyl (H, z=1).

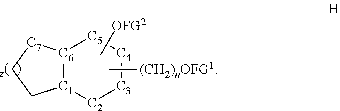

OFG¹ is preferably attached to a primary carbon, e.g., an exocyclic methylene group (n=1) or ethylene group (n=2) connected to one of C-2, C-3, C-4, or C-5 [—(CH₂)ₙOFG¹ in H]. OFG² is preferably attached directly to one of C-2, C-3, C-4, or C-5 (—OFG² in H). —(CH₂)ₙOFG¹ and OFG² may be disposed in a geminal manner on the ring, i.e., both groups may be attached to the same carbon, e.g., at C-2, C-3, C-4, or C-5. Alternatively, —(CH₂)ₙOFG¹ and OFG² may be disposed in a vicinal manner on the ring, i.e., both groups may be attached to adjacent ring carbon atoms, e.g., —(CH₂)ₙOFG¹ may be attached to C-2 and OFG² may be attached to C-3; —(CH₂)ₙOFG¹ may be attached to C-3 and OFG² may be attached to C-2; —(CH₂)ₙOFG¹ may be attached to C-3 and OFG² may be attached to C-4; or —(CH₂)ₙOFG¹ may be attached to C-4 and OFG² may be attached to C-3; —(CH₂)ₙOFG¹ may be attached to C-4 and OFG² may be attached to C-5; or —(CH₂)ₙOFG¹ may be attached to C-5 and OFG² may be attached to C-4. The decalin or indane-based monomers may therefore contain linkages (e.g., carbon-carbon bonds) wherein bond rotation is restricted about that particular linkage, e.g. restriction resulting from the presence of a ring. Thus, —(CH₂)ₙOFG¹ and OFG² may be cis or trans with respect to one another in any of the pairings delineated above. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). In a preferred embodiment, the substituents at C-1 and C-6 are trans with respect to one another. The tethering attachment point is preferably C-6 or C-7.

Other carriers may include those based on 3-hydroxyproline (J).

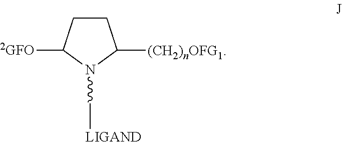

Thus, —(CH₂)ₙOFG¹ and OFG² may be cis or trans with respect to one another. Accordingly, all cis/trans isomers are expressly included. The monomers may also contain one or more asymmetric centers and thus occur as racemates and racemic mixtures, single enantiomers, individual diastereomers and diastereomeric mixtures. All such isomeric forms of the monomers are expressly included (e.g., the centers bearing CH₂OFG¹ and OFG² can both have the R configuration; or both have the S configuration; or one center can have the R configuration and the other center can have the S configuration and vice versa). The tethering attachment point is preferably nitrogen.

Details about more representative cyclic, sugar replacement-based carriers can be found in U.S. Pat. Nos. 7,745,608 and 8,017,762, which are herein incorporated by reference in their entireties.

b. Sugar Replacement-Based Monomers (A cyclic)

Acyclic sugar replacement-based monomers, e.g., sugar replacement-based ligand-conjugated monomers, are also referred to herein as ribose replacement monomer subunit (RRMS) monomer compounds. Preferred acyclic carriers can have formula LCM-3 or LCM-4:

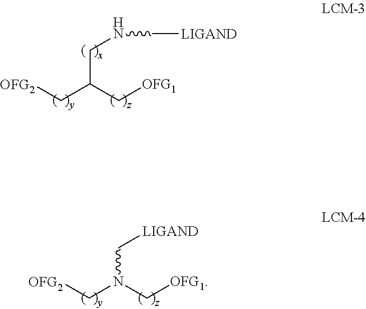

In some embodiments, each of x, y, and z can be, independently of one another, 0, 1, 2, or 3. In formula LCM-3, when y and z are different, then the tertiary carbon can have either the R or S configuration. In preferred embodiments, x is zero and y and z are each 1 in formula LCM-3 (e.g., based on serinol), and y and z are each 1 in formula LCM-3. Each of formula LCM-3 or LCM-4 below can optionally be substituted, e.g., with hydroxy, alkoxy, perhaloalkyl.

Details about more representative acyclic, sugar replacement-based carriers can be found in U.S. Pat. Nos. 7,745,608 and 8,017,762, which are herein incorporated by reference in their entireties.

The one or more C₂₂ hydrocarbon chains is conjugated to one or more internal positions on at least one strand. Internal positions of a strand refers to the nucleotide on any position of the strand, except the terminal position from the 3′ end and 5′ end of the strand (e.g., excluding 2 positions: position 1 counting from the 3′ end and position 1 counting from the 5′ end).

In one embodiment, the one or more C₂₂ hydrocarbon chains is conjugated to one or more internal positions on at least one strand, which include all positions except the terminal two positions from each end of the strand (e.g., excluding 4 positions: positions 1 and 2 counting from the 3′ end and positions 1 and 2 counting from the 5′ end). In one embodiment, the one or more Cu hydrocarbon chains is conjugated to one or more internal positions on at least one strand, which include all positions except the terminal three positions from each end of the strand (e.g., excluding 6 positions: positions 1, 2, and 3 counting from the 3′ end and positions 1, 2, and 3 counting from the 5′ end).

In one embodiment, the one or more C₂₂ hydrocarbon chains is conjugated to one or more internal positions on at least one strand, except the cleavage site region of the sense strand, for instance, the one or more C₂₂ hydrocarbon chains is not conjugated to positions 9-12 counting from the 5′-end of the sense strand, for example, the one or more C₂₂ hydrocarbon chains is not conjugated to positions 9-11 counting from the 5′-end of the sense strand. Alternatively, the internal positions exclude positions 11-13 counting from the 3′-end of the sense strand.

In one embodiment, the one or more C₂₂ hydrocarbon chains is conjugated to one or more internal positions on at least one strand, which exclude the cleavage site region of the antisense strand. For instance, the internal positions exclude positions 12-14 counting from the 5′-end of the antisense strand.

In one embodiment, the one or more C₂₂ hydrocarbon chains is conjugated to one or more internal positions on at least one strand, which exclude positions 11-13 on the sense strand, counting from the 3′-end, and positions 12-14 on the antisense strand, counting from the 5′-end.

In one embodiment, the one or more C₂₂ hydrocarbon chains is conjugated to one or more of the following internal positions: positions 4-8 and 13-18 on the sense strand, and positions 6-10 and 15-18 on the antisense strand, counting from the 5′end of each strand.

In one embodiment, the one or more C₂₂ hydrocarbon chains is conjugated to one or more of the following internal positions: positions 5, 6, 7, 15, and 17 on the sense strand, and positions 15 and 17 on the antisense strand, counting from the 5′end of each strand.

In one embodiment, the one or more C₂₂ hydrocarbon chains is conjugated to position 6 on the sense strand, counting from the 5′end of each strand.

In some embodiments, the one or more C₂₂ hydrocarbon chains is conjugated to a nucleobase, sugar moiety, or internucleosidic phosphate linkage of the dsRNA agent.

### VI. Synthesis of RNAi Agents of the Invention

The nucleic acids featured in the disclosure can be synthesized or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference.

An siRNA can be produced, e.g., in bulk, by a variety of methods. Exemplary methods include; organic synthesis and RNA cleavage, e.g., in vitro cleavage.

### A. Organic Synthesis

An siRNA can be made by separately synthesizing a single stranded RNA molecule, or each respective strand of a double-stranded RNA molecule, after which the component strands can then be annealed.

A large bioreactor, e.g., the OligoPilot II from Pharmacia Biotec AB (Uppsala Sweden), can be used to produce a large amount of a particular RNA strand for a given siRNA. The OligoPilotII reactor can efficiently couple a nucleotide using only a 1.5 molar excess of a phosphoramidite nucleotide. To make an RNA strand, ribonucleotides amidites are used. Standard cycles of monomer addition can be used to synthesize the 21 to 23 nucleotide strand for the siRNA. Typically, the two complementary strands are produced separately and then annealed, e.g., after release from the solid support and deprotection.

Organic synthesis can be used to produce a discrete siRNA species. The complementary of the species to a particular target gene can be precisely specified. For example, the species may be complementary to a region that includes a polymorphism, e.g., a single nucleotide polymorphism. Further the location of the polymorphism can be precisely defined. In some embodiments, the polymorphism is located in an internal region, e.g., at least 4, 5, 7, or 9 nucleotides from one or both of the termini.

B. dsiRNA Cleavage

siRNAs can also be made by cleaving a larger siRNA. The cleavage can be mediated in vitro or in vivo. For example, to produce iRNAs by cleavage in vitro, the following method can be used:

### 1. In Vitro Transcription.

dsiRNA is produced by transcribing a nucleic acid (DNA) segment in both directions. For example, the HiScribe™ RNAi transcription kit (New England Biolabs) provides a vector and a method for producing a dsiRNA for a nucleic acid segment that is cloned into the vector at a position flanked on either side by a T7 promoter. Separate templates are generated for T7 transcription of the two complementary strands for the dsiRNA. The templates are transcribed in vitro by addition of T7 RNA polymerase and dsiRNA is produced. Similar methods using PCR and/or other RNA polymerases (e.g., T3 or SP6 polymerase) can also be dotoxins that may contaminate preparations of the recombinant enzymes. In one embodiment, RNA generated by this method is carefully purified to remove endotoxins that may contaminate preparations of the recombinant enzymes.

### 2. In Vitro Cleavage.

dsRNA is cleaved in vitro into siRNAs, for example, using a Dicer or comparable RNAse III-based activity. For example, the dsiRNA can be incubated in an in vitro extract from Drosophila or using purified components, e.g., a purified RNAse or RISC complex (RNA-induced silencing complex). See, e.g., Ketting et al. Genes Dev 2001 Oct. 15; 15(20):2654-9 and Hammond Science 2001 Aug. 10; 293(5532):1146-50.

dsiRNA cleavage generally produces a plurality of siRNA species, each being a particular 21 to 23 nt fragment of a source dsiRNA molecule. For example, siRNAs that include sequences complementary to overlapping regions and adjacent regions of a source dsiRNA molecule may be present.

Regardless of the method of synthesis, the siRNA preparation can be prepared in a solution (e.g., an aqueous and/or organic solution) that is appropriate for formulation. For example, the siRNA preparation can be precipitated and redissolved in pure double-distilled water, and lyophilized. The dried siRNA can then be resuspended in a solution appropriate for the intended formulation process.

C. Making dsRNA Agents Conjugated to One or More C₂₂ Hydrocarbon Chains

In some embodiments, the one or more C₂₂ hydrocarbon chains is conjugated to the dsRNA agent via a nucleobase, sugar moiety, or internucleosidic linkage.

Conjugation to purine nucleobases or derivatives thereof can occur at any position including, endocyclic and exocyclic atoms. In some embodiments, the 2-, 6-, 7-, or 8-positions of a purine nucleobase are attached to a C₂₂ hydrocarbon chain. Conjugation to pyrimidine nucleobases or derivatives thereof can also occur at any position. In some embodiments, the 2-, 5-, and 6-positions of a pyrimidine nucleobase can be substituted with a C₂₂ hydrocarbon chain. When one or more Cu hydrocarbon chains is conjugated to a nucleobase, the preferred position is one that does not interfere with hybridization, i.e., does not interfere with the hydrogen bonding interactions needed for base pairing. In one embodiment, the one or more C₂₂ hydrocarbon chains may be conjugated to a nucleobase via a linker containing an alkyl, alkenyl or amide linkage.

Conjugation to sugar moieties of nucleosides can occur at any carbon atom. Exemplary carbon atoms of a sugar moiety that the one or more C₂₂ hydrocarbon chains can be attached to include the 2′, 3′, and 5′ carbon atoms. The one or more C₂₂ hydrocarbon chains can also be attached to the 1′ position, such as in an abasic residue. In one embodiment, the one or more C₂₂ hydrocarbon chains may be conjugated to a sugar moiety, via a 2′-0 modification, with or without a linker.

Internucleosidic linkages can also bear the one or more C₂₂ hydrocarbon chains. For phosphorus-containing linkages (e.g., phosphodiester, phosphorothioate, phosphorodithiotate, phosphoroamidate, and the like), the one or more C₂₂ hydrocarbon chains can be attached directly to the phosphorus atom or to an O, N, or S atom bound to the phosphorus atom. For amine- or amide-containing internucleosidic linkages (e.g., PNA), the one or more C₂₂ hydrocarbon chains can be attached to the nitrogen atom of the amine or amide or to an adjacent carbon atom.

There are numerous methods for preparing conjugates of oligonuclotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

In one embodiment, a first (complementary) RNA strand and a second (sense) RNA strand can be synthesized separately, wherein one of the RNA strands comprises a pendant C₂₂ hydrocarbon chain, and the first and second RNA strands can be mixed to form a dsRNA. The step of synthesizing the RNA strand preferably involves solid-phase synthesis, wherein individual nucleotides are joined end to end through the formation of internucleotide 3′-5′ phosphodiester bonds in consecutive synthesis cycles.

In one embodiment, the C₂₂ hydrocarbon chain having a phosphoramidite group is coupled to the 3′-end or 5′-end of either the first (complementary) or second (sense) RNA strand in the last synthesis cycle. In the solid-phase synthesis of an RNA, the nucleotides are initially in the form of nucleoside phosphoramidites. In each synthesis cycle, a further nucleoside phosphoramidite is linked to the —OH group of the previously incorporated nucleotide. If the one or more C₂₂ hydrocarbon chains has a phosphoramidite group, it can be coupled in a manner similar to a nucleoside phosphoramidite to the free OH end of the RNA synthesized previously in the solid-phase synthesis. The synthesis can take place in an automated and standardized manner using a conventional RNA synthesizer. Synthesis of the molecule having the phosphoramidite group may include phosphitylation of a free hydroxyl to generate the phosphoramidite group.

Synthesis procedures of the one or more C₂₂ hydrocarbon chain-conjugated phosphoramidites are exemplified in Example 1.

In general, the oligonucleotides can be synthesized using protocols known in the art, for example, as described in Caruthers et al., Methods in Enzymology (1992) 211:3-19; WO 99/54459; Wincott et al., Nucl. Acids Res. (1995) 23:2677-2684; Wincott et al., Methods Mol. Bio., (1997) 74:59; Brennan et al., Biotechnol. Bioeng. (1998) 61:33-45; and U.S. Pat. No. 6,001,311; each of which is hereby incorporated by reference in its entirety. In general, the synthesis of oligonucleotides involves conventional nucleic acid protecting and coupling groups, such as dimethoxytrityl at the 5′-end, and phosphoramidites at the 3′-end. In a non-limiting example, small scale syntheses are conducted on a Expedite 8909 RNA synthesizer sold by Applied Biosystems, Inc. (Weiterstadt, Germany), using ribonucleoside phosphoramidites sold by ChemGenes Corporation (Ashland, Mass.). Alternatively, syntheses can be performed on a 96-well plate synthesizer, such as the instrument produced by Protogene (Palo Alto, Calif.), or by methods such as those described in Usman et al., J. Am. Chem. Soc. (1987) 109:7845; Scaringe, et al., Nucl. Acids Res. (1990) 18:5433; Wincott, et al., Nucl. Acids Res. (1990) 23:2677-2684; and Wincott, et al., Methods Mol. Bio. (1997) 74:59, each of which is hereby incorporated by reference in its entirety.

The nucleic acid molecules of the present invention may be synthesized separately and joined together post-synthetically, for example, by ligation (Moore et al., Science (1992) 256:9923; WO 93/23569; Shabarova et al., Nucl. Acids Res. (1991) 19:4247; Bellon et al., Nucleosides & Nucleotides (1997) 16:951; Bellon et al., Bioconjugate Chem. (1997) 8:204; or by hybridization following synthesis and/or deprotection. The nucleic acid molecules can be purified by gel electrophoresis using conventional methods or can be purified by high pressure liquid chromatography (HPLC; see Wincott et al., supra, the totality of which is hereby incorporated herein by reference) and re-suspended in water.

### VII. Ligands

In certain embodiments, the dsRNA agent of the invention is further modified by covalent attachment of one or more conjugate groups. In general, conjugate groups modify one or more properties of the attached dsRNA agent of the invention including but not limited to pharmacodynamic, pharmacokinetic, binding, absorption, cellular distribution, cellular uptake, charge and clearance. Conjugate groups are routinely used in the chemical arts and are linked directly or via an optional linking moiety or linking group to a parent compound such as an oligomeric compound. A preferred list of conjugate groups includes without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes.

In some embodiments, the dsRNA agent further comprises a targeting ligand that targets a receptor which mediates delivery to a specific tissue, e.g., liver tissue. These targeting ligands can be conjugated in combination with the one or more C₂₂ hydrocarbon chains to enable specific systemic delivery. In one embodiment, a targeting ligand, e.g., one or more GalNAc derivatives, is conjugated to a dsRNA agent of the invention in combination with the one or more C₂₂ hydrocarbon chains. In another embodiment, a targeting ligand, e.g., one or more GalNAc derivatives, is not conjugated to a dsRNA agent of the invention in combination with the one or more C₂₂ hydrocarbon chains.

Exemplary targeting ligands that targets the receptor mediated delivery to an adipose tissue are peptide ligands such as Angiopep-2, lipoprotein receptor related protein (LRP) ligand, bEnd.3 cell binding ligand; transferrin receptor (TfR) ligand (which can utilize iron transport system in brain and cargo transport into the brain parenchyma); manose receptor ligand (which targets olfactory ensheathing cells, glial cells), glucose transporter protein, and LDL receptor ligand.

Preferred conjugate groups amenable to the present invention include lipid moieties such as a cholesterol moiety (Letsinger et al., Proc. Natl. Acad. Sci. USA, 1989, 86, 6553); cholic acid (Manoharan et al., Bioorg. Med. Chem. Lett., 1994, 4, 1053); a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765); a thiocholesterol (Oberhauser et al., Nucl. Acids Res., 1992, 20, 533); an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49); a phospholipid, e.g., di-hexadecyl-rac-glycerol or triethylammonium-1,2-di-O-hexadecyl-rac-glycero-3-H-phosphonate (Manoharan et al., Tetrahedron Lett., 1995, 36, 3651; Shea et al., Nucl. Acids Res., 1990, 18, 3777); a polyamine or a polyethylene glycol chain (Manoharan et al., Nucleosides & Nucleotides, 1995, 14, 969); adamantane acetic acid (Manoharan et al., Tetrahedron Lett., 1995, 36, 3651); a palmityl moiety (Mishra et al., Biochim. Biophys. Acta, 1995, 1264, 229); or an octadecylamine or hexylamino-carbonyl-oxycholesterol moiety (Crooke et al., J. Pharmacol. Exp. Ther., 1996, 277, 923).

Generally, a wide variety of entities, e.g., ligands, can be coupled to the oligomeric compounds described herein. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-101C, PEG-12K, PEG-15K, PEG-20K, PEG-40K), MPEG, [MPEG]2, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, bomeol, menthol, 1,3-propanediol, heptadecyl group, palmitic acid, myristic acid,O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a.helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), Golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and brached polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to,

(SEQ ID NO: 91)
AALEALAEALEALAEALEALAEAAAAGGC (GALA);
(SEQ ID NO: 92)
AALAEALAEALAEALAEALAEALAAAAGGC (EALA);
(SEQ ID NO: 93)
ALEALAEALEALAEA;
(SEQ ID NO: 94)
GLFEAIEGFIENGWEGMIWDYG (INF-7);
(SEQ ID NO: 95)
GLFGAIAGFIENGWEGMIDGWYG (Inf HA-2);
(SEQ ID NO: 96)
GLFEAIEGFIENGWEGMIDGWYGCGLFEAIEGFIENGWEGMID GWYGC (diINF-7);
(SEQ ID NO: 97)
GLFEAIEGFIENGWEGMIDGGCGLFEAIEGFIENGWEGMIDGGC (diINF-3);
(SEQ ID NO: 98)
GLFGALAEALAEALAEHLAEALAEALEALAAGGSC (GLF);
(SEQ ID NO: 99)
GLFEAIEGFIENGWEGLAEALAEALEALAAGGSC (GALA-INF3);
(SEQ ID NO: 100)
GLF EAI EGFI ENGW EGnI DG K GLF EAI EGFI ENGW EGnI DG (INF-5, n is norleucine);
(SEQ ID NO: 101)
LFEALLELLESLWELLLEA (JTS-1);
(SEQ ID NO: 102)
GLFKALLKLLKSLWKLLLKA (ppTG1);
(SEQ ID NO: 103)
GLFRALLRLLRSLWRLLLRA (ppTG20);
(SEQ ID NO: 104)
WEAKLAKALAKALAKHLAKALAKALKACEA (KALA);
(SEQ ID NO: 105)
GLFFEAIAEFIEGGWEGLIEGC (HA);
(SEQ ID NO: 106)
GIGAVLKVLTTGLPALISWIKRKRQQ (Melittin);
(SEQ ID NO: 107)
H5WYG;
and
(SEQ ID NO: 108)
CHK6HC.

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine (also refered to as XTC herein).

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 20070036865; and 2004/0198687, contents of which are hereby incorporated by reference in their entirety.

Exemplary cell permeation peptides include, but are not limited to,

```
(SEQ ID NO: 109)
RQIKIWFQNRRMKWKK (penetratin);
(SEQ ID NO: 110)
GRKKRRQRRRPPQC (Tat fragment 48-60);
(SEQ ID NO: 111)
GALFLGWLGAAGSTMGAWSQPKKKRKV (signal sequence based peptide);
  (SEQ ID NO: 112)
LLIILRRRIRKQAHAHSK (PVEC);
(SEQ ID NO: 113)
GWTLNSAGYLLKINLKALAALAKKIL (transportan);
(SEQ ID NO: 114)
KLALKLALKALKAALKLA (amphiphilic model peptide);
(SEQ ID NO: 115)
RRRRRRRRR (Arg9);
(SEQ ID NO: 116)
KFFKFFKFFK (Bacterial cell wall permeating peptide);
(SEQ ID NO: 117)
LLGDFFRKSKEKIGKEFKRIVQRIKDFLRNLVPRTES (LL-37);
(SEQ ID NO: 118)
SWLSKTAKKLENSAKKRISEGIAIAIQGGPR (cecropin P1);
(SEQ ID NO: 119)
ACYCRIPACIAGERRYGTCIYQGRLWAFCC (α-defensin);
(SEQ ID NO: 120)
DHYNCVSSGGQCLYSACPIFTKIQGTCYRGKAKCCK (β-defensin);
(SEQ ID NO: 121)
RRRPRPPYLPRPRPPPFFPPRLPPRIPPGFPPRFPPRFPGKR-NH2 (PR-39);
(SEQ ID NO: 122)
ILPWKWPWWPWRR-NH2 (indolicidin);
(SEQ ID NO: 123)
AAVALLPAVLLALLAP (RFGF);
  (SEQ ID NO: 124)
AALLPVLLAAP (RFGF analogue);
and
(SEQ ID NO: 125)
RKCRIVVIRVCR (bactenecin).
```

Exemplary cationic groups include, but are not limited to, protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactosamine (GalNAc), multivalent GalNAc, e.g. GalNAc₂ and GalNAc₃ (GalNAc and multivalent GalNAc are collectively referred to herein as GalNAc conjugates); D-mannose, multivalent mannose, multivalent lactose, N-acetyl-glucosamine, Glucose, multivalent Glucose, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of the composition of the invention. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (5)-(+)-pranoprofen, carprofen, PEGS, biotin, and transthyretia-binding ligands (e.g., tetraiidothyroacetic acid, 2, 4, 6-triiodophenol and flufenamic acid). Oligomeric compounds that comprise a number of phosphorothioate intersugar linkages are also known to bind to serum protein, thus short oligomeric compounds, e.g. oligonucleotides of comprising from about 5 to 30 nucleotides (e.g., 5 to 25 nucleotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleotide linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, such as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

The ligand or tethered ligand can be present on a monomer when said monomer is incorporated into a component of the dsRNA agent of the invention (e.g., a dsRNA agent of the invention or linker). In some embodiments, the ligand can be incorporated via coupling to a “precursor” monomer after said “precursor” monomer has been incorporated into a component of the dsRNA agent of the invention (e.g., a dsRNA agent of the invention or linker). For example, a monomer having, e.g., an amino-terminated tether (i.e., having no associated ligand), e.g., monomer-linker-NH₂ can be incorporated into a component of the compounds of the invention (e.g., a dsRNA agent of the invention or linker). In a subsequent operation, i.e., after incorporation of the precursor monomer into a component of the compounds of the invention (e.g., a dsRNA agent of the invention or linker), a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

In some embodiments, ligand can be conjugated to nucleobases, sugar moieties, or internucleosidic linkages of the dsRNA agent of the invention. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligonuclotides. Generally, an oligonucleotide is attached to a conjugate moiety by contacting a reactive group (e.g., OH, SH, amine, carboxyl, aldehyde, and the like) on the oligonucleotide with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

The ligand can be attached to the dsRNA agent of the inventions via a linker or a carrier monomer, e.g., a ligand carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier monomer into the backbone, e.g., the phosphate, or modified phosphate, e.g., sulfur containing, backbone, of an oligonucleotide. A “tethering attachment point” (TAP) in refers to an atom of the carrier monomer, e.g., a carbon atom or a heteroatom (distinct from an atom which provides a backbone attachment point), that connects a selected moiety. The selected moiety can be, e.g., a carbohydrate, e.g. monosaccharide, disaccharide, trisaccharide, tetrasaccharide, oligosaccharide and polysaccharide. Optionally, the selected moiety is connected by an intervening tether to the carrier monomer. Thus, the carrier will often include a functional group, e.g., an amino group, or generally, provide a bond, that is suitable for incorporation or tethering of another chemical entity, e.g., a ligand to the constituent atom.

Representative U.S. patents that teach the preparation of conjugates of nucleic acids include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,672,662; 5,688,941; 5,714,166; 6,153,737; 6,172,208; 6,300,319; 6,335,434; 6,335,437; 6,395,437; 6,444,806; 6,486,308; 6,525,031; 6,528,631; 6,559,279; contents of which are herein incorporated in their entireties by reference.

In some embodiments, the dsRNA agent further comprises a targeting ligand that targets a liver tissue. In some embodiments, the targeting ligand is a carbohydrate-based ligand. In one embodiment, the targeting ligand is a GalNAc conjugate.

In certain embodiments, the dsRNA agent of the invention further comprises a ligand having a structure shown below:

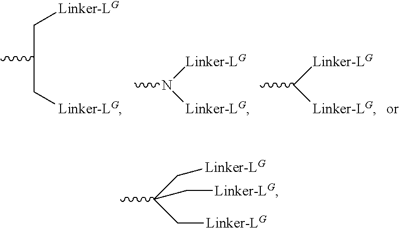

wherein:
   L^{G} is independently for each occurrence a ligand, e.g., carbohydrate, e.g. monosaccharide, disaccharide, trisaccharide, tetrasaccharide, polysaccharide; and
   Z′, Z″, Z′″ and Z″″ are each independently for each occurrence 0 or S.

In certain embodiments, the dsRNA agent of the invention comprises a ligand of Formula (II), (III), (IV) or (V):

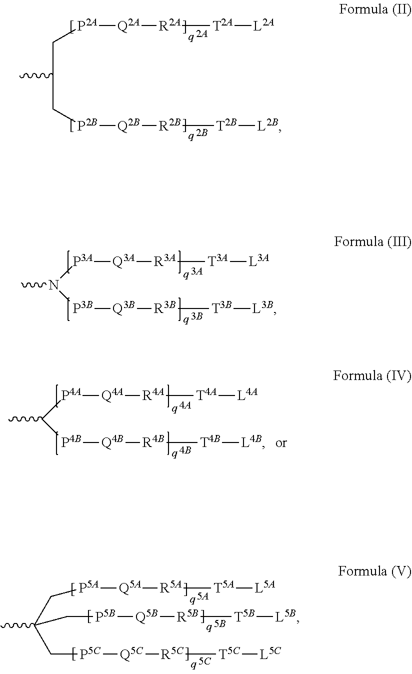

wherein:
q^{2A}, q^{2B}, q^{3A}, q^{3B}, q^{4A}, q^{4B}, q^{5A}, q^{5B} and q^{5C} represent independently for each occurrence 0-20 and wherein the repeating unit can be the same or different;
Q and Q′ are independently for each occurrence is absent, —(P⁷-Q⁷-R⁷)ₚ-T⁷- or -T⁷-Q⁷-T^{7′}-B-T^{8′}-Q⁸-T⁸;
p^{2A}, p^{2B}, p^{3A}, p^{3B}, p^{4A}, p^{4B}, p^{5A}, p^{5B}, p^{5C}, p⁷, T^{2A}, T^{2B}, T^{3A}, T^{3B}, T^{4A}, T^{4B}, T^{5A}, T^{5B}, T^{5C}, T⁷, T^{7′}, T⁸ and T^{8′} are each independently for each occurrence absent, CO, NH, O, S, OC(O), NHC(O), CH₂, CH₂NH or CH₂O;
B is —CH₂—N(B^{L})—CH₂—;
B^{L} is -T^{B}-Q^{B}-T^{B′}-R^{x};
Q^{2A}, Q^{2B}, Q^{3A}, Q^{3B}, Q^{4A}, Q^{4B}, Q^{5A}, Q^{5B}, Q^{5C}, Q⁷, Q⁸ and Q^{B} are independently for each occurrence absent, alkylene, substituted alkylene and wherein one or more methylenes can be interrupted or terminated by one or more of O, S, S(O), SO₂, N(R^{N}), C(R′)═(R′), C≡C or C(O);
T^{B} and T^{B′} are each independently for each occurrence absent, CO, NH, O, S, OC(O), OC(O)O, NHC(O), NHC(O)NH, NHC(O)O, CH₂, CH₂NH or CH₂O;
R^{x} is a lipophile (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, bomeol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, Friedelin, epifriedelanol derivatized lithocholic acid), or a cationic lipid;
R¹, R², R^{2A}, R^{2B}, R^{3A}, R^{3B}, R^{4A}, R^{4B}, R^{5A}, R^{5B}, R^{5C}, R⁷ are each independently for each occurrence absent, NH, O, S, CH₂, C(O)O, C(O)NH, NHCH(R^{a})C(O), —C(O)—CH(R^{a}) NH—, CO, CH═N—O,

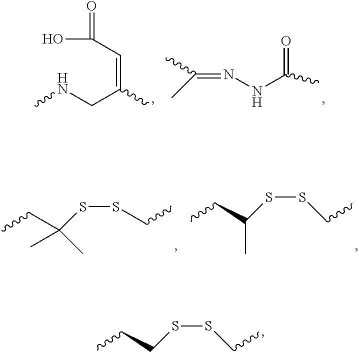

or heterocyclyl;
L¹, L^{2A}, L^{2B}, L^{3A}, L^{3B}, L^{4A}, L^{4B}, L^{5A}, L^{5B} and L^{5C} are each independently for each occurrence a carbohydrate, e.g., monosaccharide, disaccharide, trisaccharide, tetrasaccharide, oligosaccharide and polysaccharide;
R′ and R″ are each independently H, C₁-C₆ alkyl, OH, SH, or N(R^{N})₂;
R^{N} is independently for each occurrence H, methyl, ethyl, propyl, isopropyl, butyl or benzyl;
R^{a} is H or amino acid side chain;
Z′, Z″, Z′″ and Z″″ are each independently for each occurrence 0 or S;
p represent independently for each occurrence 0-20.

As discussed above, because the ligand can be conjugated to the iRNA agent via a linker or carrier, and because the linker or carrier can contain a branched linker, the iRNA agent can then contain multiple ligands via the same or different backbone attachment points to the carrier, or via the branched linker(s). For instance, the branchpoint of the branched linker may be a bivalent, trivalent, tetravalent, pentavalent, or hexavalent atom, or a group presenting such multiple valencies. In certain embodiments, the branchpoint is —N, —N(Q)-C, —O—C, —S—C, —SS—C, —C(O)N(Q)-C, —OC(O)N(Q)-C, N(Q)C(O)—C, or N(Q)C(O)O—C; wherein Q is independently for each occurrence H or optionally substituted alkyl. In other embodiment, the branchpoint is glycerol or glycerol derivative.

Suitable ligands for use in the compositions of the invention are described in U.S. Pat. Nos. 8,106,022, 8,450,467, 8,882,895, 9,352,048, 9,370,581, 9,370,582, 9,867,882, 10,806,791, and 11,110,174, and U.S. Patent Publication Nos. 2009/239814, 200/9247608, 2012/136042, 2013/178512, 2014/179761, 2015/011615, 2015/119444, 2015/119445, 2016/051691, 2016/375137, 2018/326070, 2019/099493, 2019/184018, and 2020/297853, the entire contents of each of which are incorporated herein by reference.

In some embodiments, a suitable ligand is a ligand disclosed in WO 2019/055633, the entire contents of which are incorporated herein by reference. In one embodiment the ligand comprises the structure below:

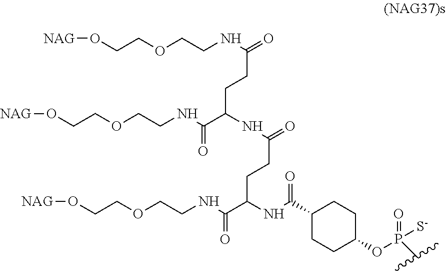

In certain embodiments, the dsRNA agent of the invention comprises a ligand of structure:

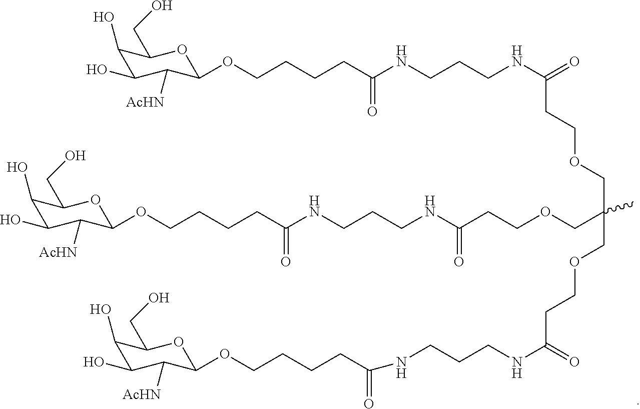 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In certain embodiments, the dsRNA agent of the invention is conjugated with a ligand of structure:

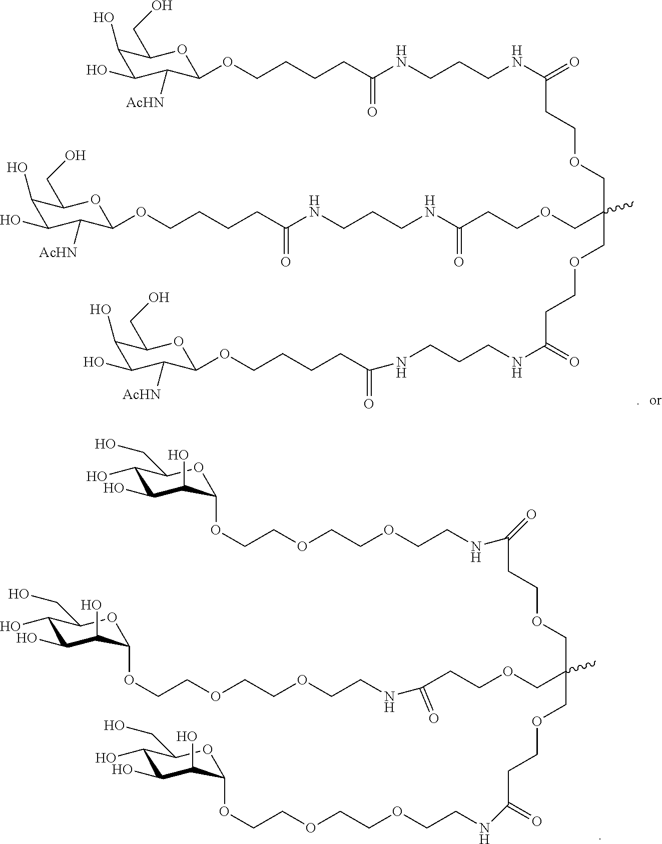

In certain embodiments, the dsRNA agent of the invention comprises a ligand of structure:

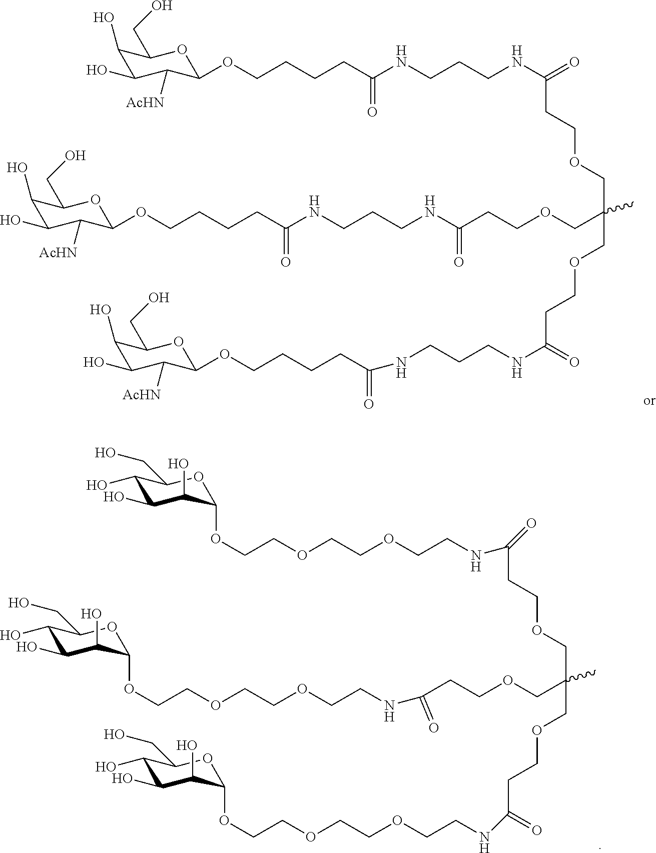

In certain embodiments, the dsRNA agent of the invention comprises a monomer of structure:

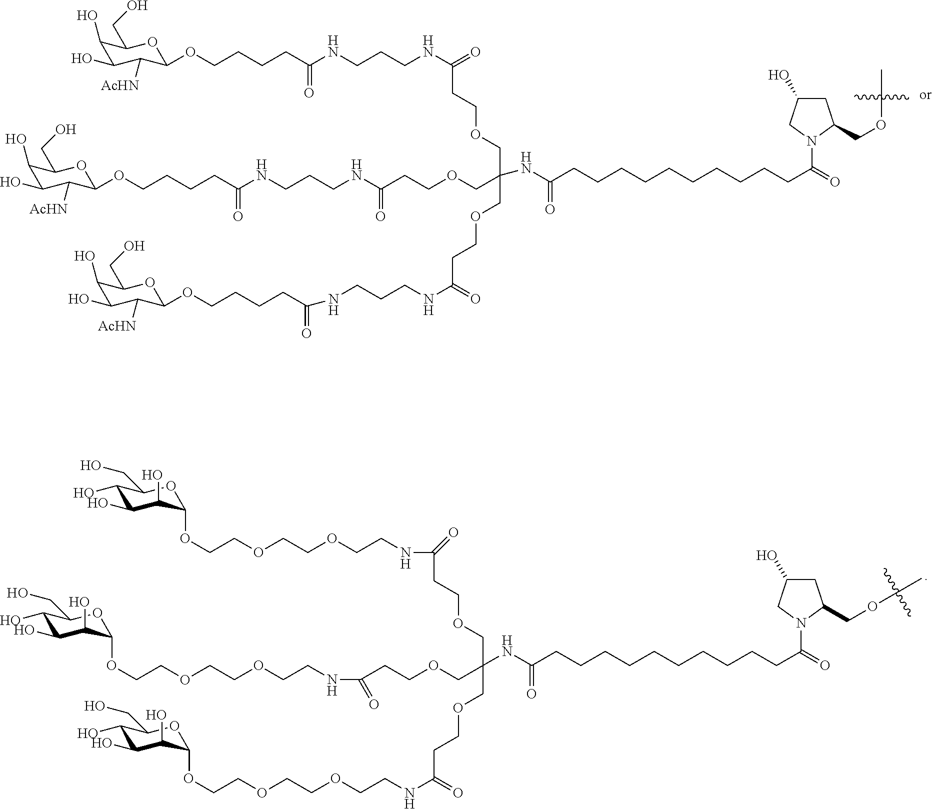

In some embodiments, the RNAi agent is attached to the carbohydrate conjugate via a linker as shown in the following schematic, wherein X is O or S

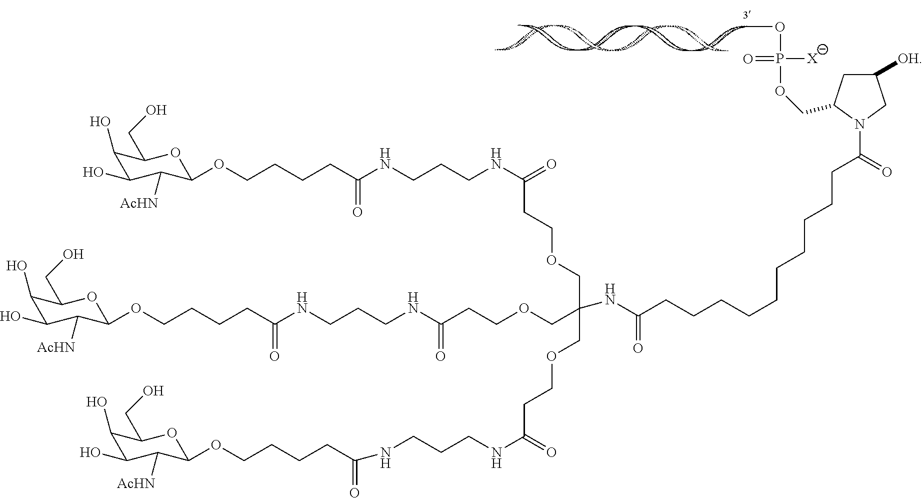

In some embodiments, the RNAi agent is conjugated to L96 as defined in Table 1 and shown below:

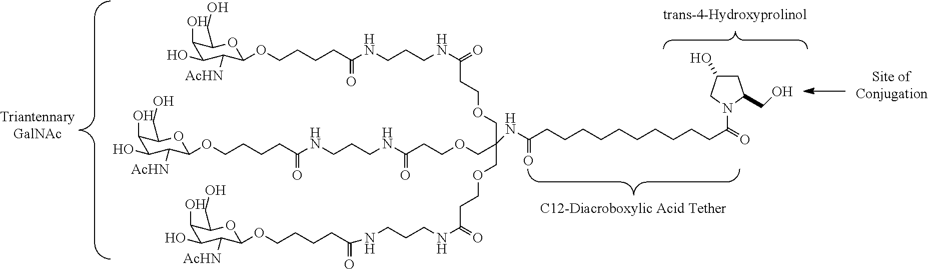

Synthesis of above described ligands and monomers is described, for example, in U.S. Pat. No. 8,106,022, content of which are incorporated herein by reference in their entirety.

### VIII. Delivery of an RNAi Agent of the Disclosure

The delivery of a RNAi agent of the disclosure to a cell e.g., a cell within a subject, such as a human subject (e.g., a subject in need thereof, such as a subject having a metabolic disorder, can be achieved in a number of different ways. For example, delivery may be performed by contacting a cell with an RNAi agent of the disclosure either in vitro or in vivo. In vivo delivery may also be performed directly by administering a composition comprising an RNAi agent, e.g., a dsRNA, to a subject. Alternatively, in vivo delivery may be performed indirectly by administering one or more vectors that encode and direct the expression of the RNAi agent. These alternatives are discussed further below.

In general, any method of delivering a nucleic acid molecule (in vitro or in vivo) can be adapted for use with a RNAi agent of the disclosure (see e.g., Akhtar S. and Julian R L., (1992) Trends Cell. Biol. 2(5):139-144 and WO94/02595, which are incorporated herein by reference in their entireties). For in vivo delivery, factors to consider in order to deliver an RNAi agent include, for example, biological stability of the delivered agent, prevention of non-specific effects, and accumulation of the delivered agent in the target tissue. The non-specific effects of an RNAi agent can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the RNAi agent to be administered. Several studies have shown successful knockdown of gene products when an RNAi agent is administered locally. For example, pulmonary delivery, e.g., inhalation, of a dsRNA, e.g., SOD1, has been shown to effectively knockdown gene and protein expression in lung tissue and that there is excellent uptake of the dsRNA by the bronchioles and alveoli of the lung. Intraocular delivery of a VEGF dsRNA by intravitreal injection in cynomolgus monkeys (Tolentino, M J. et al., (2004) Retina 24:132-138) and subretinal injections in mice (Reich, S J. et al. (2003) Mol. Vis. 9:210-216) were also both shown to prevent neovascularization in an experimental model of age-related macular degeneration. In addition, direct intratumoral injection of a dsRNA in mice reduces tumor volume (Pille, J. et al. (2005) Mol. Ther. 11:267-274) and can prolong survival of tumor-bearing mice (Kim, W J. et al., (2006) Mol. Ther. 14:343-350; Li, S. et al., (2007) Mol. Ther. 15:515-523). RNA interference has also shown success with local delivery to the CNS by direct injection (Dom, G. et al., (2004) Nucleic Acids 32:e49; Tan, P H. et al. (2005) Gene Ther. 12:59-66; Makimura, H. et al. (2002) BMC Neurosci. 3:18; Shishkina, G T., et al. (2004) Neuroscience 129:521-528; Thakker, E R., et al. (2004) Proc. Natl. Acad. Sci. USA. 101:17270-17275; Akaneya, Y., et al. (2005) J. Neurophysiol. 93:594-602) and to the lungs by intranasal administration (Howard, K A. et al., (2006) Mol. Ther. 14:476-484; Zhang, X. et al., (2004) J. Biol. Chem. 279:10677-10684; Bitko, V. et al., (2005) Nat. Med. 11:50-55). For administering a RNAi agent systemically for the treatment of a disease, the RNA can be modified or alternatively delivered using a drug delivery system; both methods act to prevent the rapid degradation of the dsRNA by endo- and exo-nucleases in vivo. Modification of the RNA or the pharmaceutical carrier can also permit targeting of the RNAi agent to the target tissue and avoid undesirable off-target effects (e.g., without wishing to be bound by theory, use of GNAs as described herein has been identified to destabilize the seed region of a dsRNA, resulting in enhanced preference of such dsRNAs for on-target effectiveness, relative to off-target effects, as such off-target effects are significantly weakened by such seed region destabilization). RNAi agents can be modified by chemical conjugation to lipophilic groups such as cholesterol to enhance cellular uptake and prevent degradation. For example, a RNAi agent directed against ApoB conjugated to a lipophilic cholesterol moiety was injected systemically into mice and resulted in knockdown of apoB mRNA in both the liver and jejunum (Soutschek, J. et al., (2004) Nature 432:173-178). Conjugation of an RNAi agent to an aptamer has been shown to inhibit tumor growth and mediate tumor regression in a mouse model of prostate cancer (McNamara, J O. et al., (2006) Nat. Biotechnol. 24:1005-1015). In an alternative embodiment, the RNAi agent can be delivered using drug delivery systems such as a nanoparticle, a dendrimer, a polymer, liposomes, or a cationic delivery system. Positively charged cationic delivery systems facilitate binding of molecule RNAi agent (negatively charged) and also enhance interactions at the negatively charged cell membrane to permit efficient uptake of an RNAi agent by the cell. Cationic lipids, dendrimers, or polymers can either be bound to an RNAi agent, or induced to form a vesicle or micelle (see e.g., Kim S H. et al., (2008) Journal of Controlled Release 129(2):107-116) that encases an RNAi agent. The formation of vesicles or micelles further prevents degradation of the RNAi agent when administered systemically. Methods for making and administering cationic-RNAi agent complexes are well within the abilities of one skilled in the art (see e.g., Sorensen, D R., et al. (2003) J. Mot Biol 327:761-766; Verma, U N. et al., (2003) Clin. Cancer Res. 9:1291-1300; Arnold, A S et al. (2007) J. Hypertens. 25:197-205, which are incorporated herein by reference in their entirety). Some non-limiting examples of drug delivery systems useful for systemic delivery of RNAi agents include DOTAP (Sorensen, D R., et al (2003), supra; Verma, U N. et al., (2003), supra), Oligofectamine, “solid nucleic acid lipid particles” (Zimmermann, T S. et at, (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, a RNAi agent forms a complex with cyclodextrin for systemic administration. Methods for administration and pharmaceutical compositions of RNAi agents and cyclodextrins can be found in U.S. Patent No. 7, 427, 605, which is herein incorporated by reference in its entirety.

Certain aspects of the instant disclosure relate to a method of reducing the expression of a target gene, e.g., INHBE, ACVR1C, PLIN1, PDE3B, or INHBC, in a cell, comprising contacting said cell with the double-stranded RNAi agent of the disclosure. In one embodiment, the cell is a liver cell. In one embodiment, the cell is an adipocyte.

In certain embodiments, the RNAi agent is taken up on one or more tissue or cell types present in organs, e.g., liver, adipose tissue.

Another aspect of the disclosure relates to a method of reducing the expression and/or activity of a target gene, e.g., INHBE, ACVR1C, PLIN1, PDE3B, or INHBC, in a subject, comprising administering to the subject the double-stranded RNAi agent of the disclosure.

Another aspect of the disclosure relates to a method of treating a subject having a metabolic disorder or at risk of having or at risk of developing a metabolic disorder, comprising administering to the subject a therapeutically effective amount of the double-stranded RNAi agent of the disclosure, thereby treating the subject.

In one embodiment, the double-stranded RNAi agent is administered subcutaneously.

In one embodiment, the double-stranded RNAi agent is administered intramuscularly.

In one embodiment, the double-stranded RNAi agent is administered by intravenously.

In one embodiment, the double-stranded RNAi agent is administered by pulmonary system administration, e.g., intranasal administration, or oral inhalative administration.

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the disclosure. A composition that includes a RNAi agent can be delivered to a subject by a variety of routes. Exemplary mutes include pulmonary system, intravenous, subcutaneous, intraventricular, oral, topical, rectal, anal, vaginal, nasal, and ocular.

The RNAi agents of the disclosure can be incorporated into pharmaceutical compositions suitable for administration. Such compositions typically include one or more species of RNAi agent and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present disclosure may be administered in a number of ways depending upon whether local or systemic treatment is desired and upon the area to be treated. Administration may be intratracheal, intranasal, topical (including ophthalmic, vaginal, rectal, intranasal, transdermal), oral, parenteral, or pulmonary, e.g., by inhalation or insufflation of powders or aerosols, including by nebulizer. Parenteral administration includes intravenous drip, subcutaneous, intraperitoneal, or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering the RNAi agent in powder or aerosol form. The vascular endothelial cells could be targeted by coating a balloon catheter with the RNAi agent and mechanically introducing the RNA.

Compositions for pulmonary system delivery may include aqueous solutions, e.g., for intranasal or oral inhalative administration, suitable carriers composed of, e.g., lipids (liposomes, niosomes, microemulsions, lipidic micelles, solid lipid nanoparticles) or polymers (polymer micelles, dendrimers, polymeric nanoparticles, nonogels, nanocapsules), adjuvant, e.g., for oral inhalative administration. Aqueous compositions may be sterile and may optionally contain buffers, diluents, absorption enhancers and other suitable additives. Such administration permits both systemic and local delivery of the double stranded RNAi agents of the invention.

Intranasal administration may include instilling or insufflating a double stranded RNAi agent into the nasal cavity with syringes or droppers by applying a few drops at a time or via atomization. Suitable dosage forms for intranasal administration include drops, powders, nebulized mists, and sprays. Nasal delivery devices include, but not limited to, vapor inhaler, nasal dropper, spray bottle, metered dose spray pump, gas driven spray atomizer, nebulizer, mechanical powder sprayer, breath actuated inhaler, and insufflator. Devices for delivery deeper into the respiratory system, e.g., into the lung, include nebulizer, pressured metered-dose inhaler, dry powder inhaler, and thermal vaporization aerosol device. Devices for delivery by inhalation are available from commercial suppliers. Devices can be fixed or variable dose, single or multidose, disposable or reusable depending on, for example, the disease or disorder to be prevented or treated, the volume of the agent to be delivered, the frequency of delivery of the agent, and other considerations in the art.

Oral inhalative administration may include use of device, e.g., a passive breath driven or active power driven single/-multiple dose dry powder inhaler (DPI), to deliver a double stranded RNAi agent to the pulmonary system. Suitable dosage forms for oral inhalative administration include powders and solutions. Suitable devices for oral inhalative administration include nebulizers, metered-dose inhalers, and dry powder inhalers. Dry powder inhalers are of the most popular devices used to deliver drugs, especially proteins to the lungs. Exemplary commercially available dry powder inhalers include Spinhaler (Fisons Pharmaceuticals, Rochester, NY) and Rotahaler (GSK, RTP, NC). Several types of nebulizers are available, namely jet nebulizers, ultrasonic nebulizers, vibrating mesh nebulizers. Jet nebulizers are driven by compressed air. Ultrasonic nebulizers use a piezoelectric transducer in order to create droplets from an open liquid reservoir. Vibrating mesh nebulizers use perforated membranes actuated by an annular piezoelement to vibrate in resonant bending mode. The holes in the membrane have a large cross-section size on the liquid supply side and a narrow cross-section size on the side from where the droplets emerge. Depending on the therapeutic application, the hole sizes and number of holes can be adjusted. Selection of a suitable device depends on parameters, such as nature of the drug and its formulation, the site of action, and pathophysiology of the lung. Aqueous suspensions and solutions are nebulized effectively. Aerosols based on mechanically generated vibration mesh technologies also have been used successfully to deliver proteins to lungs.

The amount of RNAi agent for pulmonary system administration may vary from one target gene to another target gene and the appropriate amount that has to be applied may have to be determined individually for each target gene. Typically, this amount ranges from 10 μg to 2 mg, or 50 μg to 1500 μg, or 100 μg to 1000 μg.

Formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable. Coated condoms, gloves, and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening or flavoring agents can be added. Compositions suitable for oral administration of the agents of the invention are further described in PCT Application No. PCT/US20/33156, the entire contents of which are incorporated herein by reference.

Compositions for intrathecal or intraventricular administration may include sterile aqueous solutions which may also contain buffers, diluents, and other suitable additives.

Formulations for parenteral administration may include sterile aqueous solutions which may also contain buffers, diluents, and other suitable additives. Intraventricular injection may be facilitated by an intraventricular catheter, for example, attached to a reservoir. For intravenous use, the total concentration of solutes may be controlled to render the preparation isotonic.

In one embodiment, the administration of the siRNA compound, e.g., a double-stranded siRNA compound, is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary system, intranasal, urethral, or ocular. Administration can be provided by the subject or by another person, e.g., a health care provider. The medication can be provided in measured doses or in a dispenser which delivers a metered dose. Selected modes of delivery are discussed in more detail below.

### A. Vector Encoded RNAi Agents of the Disclosure

RNAi agents targeting the target gene can be expressed from transcription units inserted into DNA or RNA vectors (see, e.g., Couture, A, et al., TIG. (1996), 12:5-10; WO 00/22113, WO 00/22114, and U.S. Pat. No. 6,054,299). Expression can be sustained (months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., (1995) Proc. Natl. Acad. Sci. USA 92:1292).

The individual strand or strands of a RNAi agent can be transcribed from a promoter on an expression vector. Where two separate strands are to be expressed to generate, for example, a dsRNA, two separate expression vectors can be co-introduced (e.g., by transfection or infection) into a target cell. Alternatively, each individual strand of a dsRNA can be transcribed by promoters both of which are located on the same expression plasmid. In one embodiment, a dsRNA is expressed as inverted repeat polynucleotides joined by a linker polynucleotide sequence such that the dsRNA has a stem and loop structure.

RNAi agent expression vectors are generally DNA plasmids or viral vectors. Expression vectors compatible with eukaryotic cells, such as those compatible with vertebrate cells, can be used to produce recombinant constructs for the expression of a RNAi agent as described herein. Delivery of RNAi agent expressing vectors can be systemic, such as by intravenous or intramuscular administration, by administration to target cells ex-planted from the patient followed by reintroduction into the patient, or by any other means that allows for introduction into a desired target cell.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. Constructs for the recombinant expression of a RNAi agent will generally require regulatory elements, e.g., promoters, enhancers, etc., to ensure the expression of the RNAi agent in target cells. Other aspects to consider for vectors and constructs are known in the art.

### IX. Pharmaceutical Compositions

The present disclosure also includes pharmaceutical compositions and formulations which include the RNAi agents of the disclosure. In one embodiment, provided herein are pharmaceutical compositions containing an RNAi agent, as described herein, and a pharmaceutically acceptable carrier. The pharmaceutical compositions containing the RNAi agent are useful for treating a subject who would benefit from inhibiting or reducing the expression of a target gene, e.g., INHBE, ACVR1C, PLIN1, PDE3B, or INHBC, e.g., a subject having a metabolic disorder. Such pharmaceutical compositions are formulated based on the mode of delivery. One example is compositions that are formulated for systemic administration via parenteral delivery, e.g., by intravenous (IV), intramuscular (IM), or for subcutaneous (subQ) delivery.

In some embodiments, the pharmaceutical compositions of the invention are pyrogen free or non-pyrogenic.

In one embodiment, the delivery vehicle can deliver an iRNA compound, e.g., a double-stranded iRNA compound, or ssiRNA compound, (e.g., a precursor thereof, e.g., a larger siRNA compound which can be processed into a ssiRNA compound, or a DNA which encodes an siRNA compound, e.g., a double-stranded siRNA compound, or ssiRNA compound, or precursor thereof) to a cell by a topical route of administration. The delivery vehicle can be microscopic vesicles. In one example the microscopic vesicles are liposomes. In some embodiments the liposomes are cationic liposomes. In another example the microscopic vesicles are micelles. In one aspect, the invention features a pharmaceutical composition including an siRNA compound, e.g., a double-stranded siRNA compound, or ssiRNA compound, (e.g., a precursor thereof, e.g., a larger siRNA compound which can be processed into a ssiRNA compound, or a DNA which encodes an siRNA compound, e.g., a double-stranded siRNA compound, or ssiRNA compound, or precursor thereof) in an injectable dosage form. In one embodiment, the injectable dosage form of the pharmaceutical composition includes sterile aqueous solutions or dispersions and sterile powders. In some embodiments the sterile solution can include a diluent such as water; saline solution; fixed oils, polyethylene glycols, glycerin, or propylene glycol.

In one aspect, the invention features a pharmaceutical composition including an siRNA compound, e.g., a double-stranded siRNA compound, or ssiRNA compound, (e.g., a precursor thereof, e.g., a larger siRNA compound which can be processed into a ssiRNA compound, or a DNA which encodes an siRNA compound, e.g., a double-stranded siRNA compound, or ssiRNA compound, or precursor thereof) in oral dosage form. In one embodiment, the oral dosage form is selected from the group consisting of tablets, capsules and gel capsules. In another embodiment, the pharmaceutical composition includes an enteric material that substantially prevents dissolution of the tablets, capsules or gel capsules in a mammalian stomach. In some embodiments the enteric material is a coating. The coating can be acetate phthalate, propylene glycol, sorbitan monoleate, cellulose acetate trimellitate, hydroxy propyl methyl cellulose phthalate or cellulose acetate phthalate. In one embodiment, the oral dosage form of the pharmaceutical composition includes a penetration enhancer, e.g., a penetration enhancer described herein.

In another embodiment, the oral dosage form of the pharmaceutical composition includes an excipient. In one example the excipient is polyethyleneglycol. In another example the excipient is precirol.

In another embodiment, the oral dosage form of the pharmaceutical composition includes a plasticizer. The plasticizer can be diethyl phthalate, triacetin dibutyl sebacate, dibutyl phthalate or triethyl citrate.

### X. Methods for Inhibiting Target Gene Expression

Another aspect of the invention relates to a method of reducing the expression of a target gene, e.g., INHBE, ACVR1C, PLIN1, PDE3B, or INHBC, in a cell, comprising contacting the cell with a dsRNA agent of the invention. The methods include contacting the cell with a dsRNA of the disclosure and maintaining the cell for a time sufficient to obtain degradation of the mRNA transcripts of a target gene, thereby inhibiting expression of the target gene in the cell.

Reduction in gene expression can be assessed by any methods known in the art. For example, a reduction in the expression of a target may be determined by determining the mRNA expression level of the target gene using methods routine to one of ordinary skill in the art, e.g., northern blotting, qRT-PCR; by determining the protein level of a target protein using methods routine to one of ordinary skill in the art, such as western blotting, immunological techniques.

In the methods of the disclosure the cell may be contacted in vitro or in vivo, i.e., the cell may be within a subject. Contacting a cell in vivo with the RNAi agent includes contacting a cell or group of cells within a subject, e.g., a human subject, with the RNAi agent. Combinations of in vitro and in vivo methods of contacting a cell are also possible.

The cell may be an extra-hepatic cell, such as a liver cell or an adipocyte.

A cell suitable for treatment using the methods of the disclosure may be any cell that expresses a target gene. A cell suitable for use in the methods of the disclosure may be a mammalian cell, e.g., a primate cell (such as a human cell or a non-human primate cell, e.g., a monkey cell or a chimpanzee cell), a non-primate cell (such as a rat cell, or a mouse cell. In one embodiment, the cell is a human cell, e.g., a human liver cell or a human kidney cell.

Contacting a cell may be direct or indirect, as discussed above. Furthermore, contacting a cell may be accomplished via a targeting ligand, including any ligand described herein or known in the art. In some embodiments, the targeting ligand is a carbohydrate moiety, e.g., a GalNAc ligand, or any other ligand that directs the RNAi agent to a site of interest. In certain embodiments, the RNAi agent does not include a targeting ligand.

The term “inhibiting,” as used herein, is used interchangeably with “reducing,” “silencing,” “downregulating,” “suppressing” and other similar terms, and includes any level of inhibition. In certain embodiments, a level of inhibition, e.g., for an RNAi agent of the instant disclosure, can be assessed in cell culture conditions, e.g., wherein cells in cell culture are transfected via Lipofectamine™-mediated transfection at a concentration in the vicinity of a cell of 10 nM or less, 1 nM or less, etc. Knockdown of a given RNAi agent can be determined via comparison of pre-treated levels in cell culture versus post-treated levels in cell culture, optionally also comparing against cells treated in parallel with a scrambled or other form of control RNAi agent. Knockdown in cell culture of, e.g., 50% or more, can thereby be identified as indicative of “inhibiting” or “reducing”, “downregulating” or “suppressing”, etc. having occurred. It is expressly contemplated that assessment of targeted mRNA or encoded protein levels (and therefore an extent of “inhibiting”, etc. caused by a RNAi agent of the disclosure) can also be assessed in in vivo systems for the RNAi agents of the instant disclosure, under properly controlled conditions as described in the art.

The phrase “inhibiting expression of a target gene” or “inhibiting expression of a target,” as used herein, includes inhibition of expression of any target gene (such as, e.g., a mouse target gene, a rat target gene, a monkey target gene, or a human target gene) as well as variants or mutants of a target gene that encode a target protein. Thus, the target gene may be a wild-type target gene, a mutant target gene, or a transgenic target gene in the context of a genetically manipulated cell, group of cells, or organism.

“Inhibiting expression of a target gene” includes any level of inhibition of a target gene, e.g., at least partial suppression of the expression of a target gene, such as an inhibition by at least 20%. In certain embodiments, inhibition is by at least 30%, at least 40%, at least 50%, at least about 60%, at least 70%, at least about 80%, at least 85%, at least 90%, at least 95%, at least 96%, at least 97%, at least 98%, or at least 99%; or to below the level of detection of the assay method. In certain method, inhibition is measured at a 10 nM concentration of the siRNA using the luciferase assay provided in Example 1.

The expression of a target gene may be assessed based on the level of any variable associated with target gene expression, e.g., target mRNA level or target protein level.

Inhibition may be assessed by a decrease in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

In some embodiments of the methods of the disclosure, expression of a target gene is inhibited by at least 20%, 30%, 40%, 50%, 60%, 70%, 80%, 85%, 90%, or 95%, or to below the level of detection of the assay. In certain embodiments, the methods include a clinically relevant inhibition of expression of a target gene, e.g. as demonstrated by a clinically relevant outcome after treatment of a subject with an agent to reduce the expression of a target gene.

Inhibition of the expression of a target gene may be manifested by a reduction of the amount of mRNA expressed by a first cell or group of cells (such cells may be present, for example, in a sample derived from a subject) in which a target gene is transcribed and which has or have been treated (e.g., by contacting the cell or cells with a RNAi agent of the disclosure, or by administering a RNAi agent of the disclosure to a subject in which the cells are or were present) such that the expression of a target gene is inhibited, as compared to a second cell or group of cells substantially identical to the first cell or group of cells but which has not or have not been so treated (control cell(s) not treated with a RNAi agent or not treated with a RNAi agent targeted to the genome of interest). The degree of inhibition may be expressed in terms of:

$$\frac{( mRNA in control cells ) - ( mRNA in treated cells )}{( mRNA in control cells )} \cdot 100 \%$$

In other embodiments, inhibition of the expression of a target gene may be assessed in terms of a reduction of a parameter that is functionally linked to a target gene expression, e.g., target protein expression. Target gene silencing may be determined in any cell expressing a target gene, either endogenous or heterologous from an expression construct, and by any assay known in the art.

Inhibition of the expression of a target protein may be manifested by a reduction in the level of the target protein that is expressed by a cell or group of cells (e.g., the level of protein expressed in a sample derived from a subject). As explained above, for the assessment of genome suppression, the inhibition of protein expression levels in a treated cell or group of cells may similarly be expressed as a percentage of the level of protein in a control cell or group of cells.

A control cell or group of cells that may be used to assess the inhibition of the expression of a target gene includes a cell or group of cells that has not yet been contacted with an RNAi agent of the disclosure. For example, the control cell or group of cells may be derived from an individual subject (e.g., a human or animal subject) prior to treatment of the subject with an RNAi agent.

The level of target gene mRNA that is expressed by a cell or group of cells may be determined using any method known in the art for assessing RNA expression. In one embodiment, the level of expression of target gene in a sample is determined by detecting a transcribed polynucleotide, or portion thereof, e.g., mRNA of the target gene. RNA may be extracted from cells using RNA extraction techniques including, for example, using acid phenol/guanidine isothiocyanate extraction (RNAzol B; Biogenesis), RNeasym™ RNA preparation kits (Qiagen®) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating target mRNA may be detected using methods the described in WO2012/177906, the entire contents of which are hereby incorporated herein by reference.

In some embodiments, the level of expression of target gene is determined using a nucleic acid probe. The term “probe”, as used herein, refers to any molecule that is capable of selectively binding to a specific target nucleic acid or protein, or fragment thereof. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses and probe arrays. One method for the determination of RNA levels involves contacting the isolated RNA with a nucleic acid molecule (probe) that can hybridize to target RNA. In one embodiment, the RNA is immobilized on a solid surface and contacted with a probe, for example by running the isolated RNA on an agarose gel and transferring the RNA from the gel to a membrane, such as nitrocellulose. In an alternative embodiment, the probe(s) are immobilized on a solid surface and the RNA is contacted with the probe(s), for example, in an Affymetrix® gene chip array. A skilled artisan can readily adapt known RNA detection methods for use in determining the level of target mRNA.

An alternative method for determining the level of expression of target in a sample involves the process of nucleic acid amplification or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the disclosure, the level of expression of target is determined by quantitative fluorogenic RT-PCR (i.e., the TagMan™ System), by a Dual-Glo® Luciferase assay, or by other art-recognized method for measurement of target expression or mRNA level.

The expression level of target mRNA may be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The determination of target expression level may also comprise using nucleic acid probes in solution.

In some embodiments, the level of RNA expression is assessed using branched DNA (bDNA) assays or real time PCR (qPCR). The use of this PCR method is described and exemplified in the Examples presented herein. Such methods can also be used for the detection of target nucleic acids.

The level of target protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of target proteins.

In some embodiments, the efficacy of the methods of the disclosure in the treatment of a target gene-related disease is assessed by a decrease in target mRNA level (e.g, by assessment of a blood target gene level, or otherwise).

In some embodiments, the efficacy of the methods of the disclosure in the treatment of a target gene-related disease is assessed by a decrease in target mRNA level (e.g, by assessment of a liver or kidney sample for target level, by biopsy, or otherwise).

In some embodiments of the methods of the disclosure, the RNAi agent is administered to a subject such that the RNAi agent is delivered to a specific site within the subject. The inhibition of expression of target may be assessed using measurements of the level or change in the level of target mRNA or target protein in a sample derived from a specific site within the subject, e.g., liver or kidney cells. In certain embodiments, the methods include a clinically relevant inhibition of expression of target, e.g. as demonstrated by a clinically relevant outcome after treatment of a subject with an agent to reduce the expression of target gene.

As used herein, the terms detecting or determining a level of an analyte are understood to mean performing the steps to determine if a material, e.g., protein, RNA, is present. As used herein, methods of detecting or determining include detection or determination of an analyte level that is below the level of detection for the method used.

### XI. Prophylactic and Treatment Methods of the Invention

The present invention also provides methods of using an iRNA of the invention or a composition containing an iRNA of the invention to inhibit expression of a metabolic disorder-associated target gene, thereby preventing or treating a metabolic disorder, e.g., metabolic syndrome, a disorder of carbohydrates, e.g., type II diabetes, pre-diabetes, a lipid metabolism disorder, e.g., a hyperlipidemia, hypertension, lipodystrophy; a kidney disease; a cardiovascular disease, a disorder of body weight. In the methods of the invention the cell may be contacted with the siRNA in vitro or in vivo, i.e., the cell may be within a subject.

A cell suitable for treatment using the methods of the invention may be any cell that expresses a metabolic disorder-associated target gene, e.g., INHBE, ACVR1C, PLIN1, PDE3B, or INHBC, e.g., an adipocyte cell, or a liver cell. A cell suitable for use in the methods of the invention may be a mammalian cell, e.g., a primate cell (such as a human cell, including human cell in a chimeric non-human animal, or a non-human primate cell, e.g., a monkey cell or a chimpanzee cell), or a non-primate cell. In certain embodiments, the cell is a human cell, e.g., a human liver cell. In the methods of the invention, target gene expression is inhibited in the cell by at least 50, 55, 60, 65, 70, 75, 80, 85, 90, or 95, or to a level below the level of detection of the assay.

The in vivo methods of the invention may include administering to a subject a composition containing an iRNA, where the iRNA includes a nucleotide sequence that is complementary to at least a part of an RNA transcript of the target gene of the mammal to which the RNAi agent is to be administered. The composition can be administered by any means known in the art including, but not limited to oral, intraperitoneal, or parenteral routes, including intracranial (e.g., intraventricular, intraparenchymal, and intrathecal), intravenous, intramuscular, subcutaneous, transdermal, airway (aerosol), nasal, rectal, intraocular (e.g., periocular, conjunctival, subtenon, intracameral, intravitreal, intraocular, anterior or posterior juxtascleral, subretinal, subconjunctival, retrobulbar, or intracanalicular injection), intravenous, intramuscular, subcutaneous, transdermal, airway (aerosol), and topical (including buccal and sublingual) administration.

In certain embodiments, the compositions are administered by intravenous infusion or injection. In certain embodiments, the compositions are administered by subcutaneous injection. In certain embodiments, the compositions are administered by intramuscular injection.

The mode of administration may be chosen based upon whether local or systemic treatment is desired and based upon the area to be treated. The route and site of administration may be chosen to enhance targeting.

In one aspect, the present invention also provides methods for inhibiting the expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC) in a mammal. The methods include administering to the mammal a composition comprising a dsRNA that targets a target gene in a cell of the mammal and maintaining the mammal for a time sufficient to obtain degradation of the mRNA transcript of the target gene, thereby inhibiting expression of the target gene in the cell. Reduction in gene expression can be assessed by any methods known in the art and by methods, e.g. qRT-PCR, described herein, e.g., in Example 2. Reduction in protein production can be assessed by any methods known it the art, e.g. ELISA. In certain embodiments, a puncture liver biopsy sample serves as the tissue material for monitoring the reduction in the target gene or protein expression. In other embodiments, a blood sample serves as the subject sample for monitoring the reduction in the target protein expression.

The present invention further provides methods of treatment in a subject in need thereof, e.g., a subject diagnosed with a metabolic disorder, e.g., metabolic syndrome, a disorder of carbohydrates, e.g., type II diabetes, pre-diabetes, a lipid metabolism disorder, e.g., a hyperlipidemia, hypertension, lipodystrophy; a kidney disease; a cardiovascular disease, a disorder of body weight.

The present invention further provides methods of prophylaxis in a subject in need thereof. The treatment methods of the invention include administering an iRNA of the invention to a subject, e.g., a subject that would benefit from a reduction of expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC), in a prophylactically effective amount of a dsRNA targeting INHBE, ACVR1C, PLIN1, PDE3B, or INHBC or a pharmaceutical composition comprising a dsRNA targeting INHBE, ACVR1C, PLIN1, PDE3B, or INHBC.

In one aspect, the present invention provides methods of treating a subject having a disorder that would benefit from reduction in expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC), e.g., a metabolic disorder, e.g., diabetes.

Treatment of a subject that would benefit from a reduction and/or inhibition of INHBE, ACVR1C, PLIN1, PDE3B, or INHBC gene expression includes therapeutic treatment (e.g., a subject is having a metabolic disorder) and prophylactic treatment (e.g., the subject is not having a metabolic disorder or a subject may be at risk of developing a metabolic disorder).

Examples of metabolic disorders include but are not limited to, metabolic syndrome, a disorder of carbohydrates, e.g., type II diabetes, pre-diabetes, a lipid metabolism disorder, e.g., a hyperlipidemia, hypertension, lipodystrophy; a kidney disease; a cardiovascular disease, a disorder of body weight.

In some embodiments, the metabolic disorder is metabolic syndrome.

In some embodiments, the RNAi agent is administered to a subject in an amount effective to inhibit expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC) in a cell within the subject. The amount effective to inhibit target gene expression in a cell within a subject may be assessed using methods discussed above, including methods that involve assessment of the inhibition of target gene mRNA, target gene protein, or related variables, such as insulin resistance, BMI, WHRadj BMI, adipose tissue, e.g., image-based quantification of adipose tissue, e.g., MRI or DEXA for abdominal subcutaneous adipose and visceral adipose tissue quantification.

An iRNA of the invention may be administered as a “free iRNA.” A free iRNA is administered in the absence of a pharmaceutical composition. The naked iRNA may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution containing the iRNA can be adjusted such that it is suitable for administering to a subject.

Alternatively, an iRNA of the invention may be administered as a pharmaceutical composition, such as a dsRNA liposomal formulation.

Subjects that would benefit from an inhibition of INHBE, ACVR1C, PLIN1, PDE3B, or INHBC gene expression are subjects susceptible to or diagnosed with a metabolic disorder, e.g., metabolic syndrome, a disorder of carbohydrates, e.g., type II diabetes, pre-diabetes, a lipid metabolism disorder, e.g., a hyperlipidemia, hypertension, lipodystrophy; a kidney disease; a cardiovascular disease, a disorder of body weight. In an embodiment, the method includes administering a composition featured herein such that expression of the target gene is decreased, such as for about 1, 2, 3, 4, 5, 6, 1-6, 1-3, or 3-6 months per dose. In certain embodiments, the composition is administered once every 3-6 months.

In one embodiment, the iRNAs useful for the methods and compositions featured herein specifically target RNAs (primary or processed) of the target gene. Compositions and methods for inhibiting the expression of these genes using iRNAs can be prepared and performed as described herein.

Administration of the iRNA according to the methods of the invention may result prevention or treatment of a metabolic disorder, e.g., metabolic syndrome, a disorder of carbohydrates, e.g., type II diabetes, pre-diabetes, a lipid metabolism disorder, e.g., a hyperlipidemia, hypertension, lipodystrophy; a kidney disease; a cardiovascular disease, a disorder of body weight. Subjects can be administered a therapeutic amount of iRNA, such as about 0.01 mg/kg to about 200 mg/kg.

In one embodiment, the iRNA is administered subcutaneously, i.e., by subcutaneous injection. One or more injections may be used to deliver the desired dose of iRNA to a subject. The injections may be repeated over a period of time.

The administration may be repeated on a regular basis. In certain embodiments, after an initial treatment regimen, the treatments can be administered on a less frequent basis. A repeat-dose regimen may include administration of a therapeutic amount of iRNA on a regular basis, such as once per month to once a year. In certain embodiments, the iRNA is administered about once per month to about once every three months, or about once every three months to about once every six months.

The invention further provides methods and uses of an iRNA agent or a pharmaceutical composition thereof for treating a subject that would benefit from reduction and/or inhibition of INHBE, ACVR1C, PLIN1, PDE3B, or INHBC gene expression, e.g., a subject having a metabolic disorder, in combination with other pharmaceuticals and/or other therapeutic methods, e.g., with known pharmaceuticals and/or known therapeutic methods, such as, for example, those which are currently employed for treating these disorders.

Accordingly, in some aspects of the invention, the methods which include administration of an iRNA agent of the invention, further include administering to the subject one or more additional therapeutic agents.

For example, in certain embodiments, an iRNA targeting INHBE, ACVR1C, PLIN1, PDE3B, or INHBC is administered in combination with, e.g., an agent useful in treating a metabolic disorder as described herein or otherwise known in the art. For example, additional agents and treatments suitable for treating a subject that would benefit from reduction in INHBE, ACVR1C, PLIN1, PDE3B, or INHBC expression, e.g., a subject having a metabolic disorder, may include agents currently used to treat symptoms of a metabolic disorder.

Examples of the additional therapeutic agents which can be used with an RNAi agent of the invention include, but are not limited to, insulin, a glucagon-like peptide 1 agonist (e.g., exenatide, liraglutide, dulaglutide, semaglutide, and pramlintide, a sulfonylurea (e.g., chlorpropamide, glipizide), a seglitinide (e.g., repaglinide, nateglinidie), biguanides (e.g., metformin), a thiazolidinedione, e.g, rosiglitazone, troglitazone, an alpha-glucosidase inhibitor (e.g., acarbose and meglitol), an SGLT2 inhibitor (e.g., dapagliflozin), a DPP-4 inhibitor (e.g., linagliptin), or an HMG-CoA reductase inhibitor, e.g., statins, such as atorvastatin (Lipitor), fluvastatin (Lescol), lovastatin (Mevacor), lovastatin extended-release (Altoprev), pitavastatin (Livalo), pravastatin (Pravachol), rosuvastatin (Crestor), and simvastatin (Zocor).

The method according to any one of claims 34 to 36, wherein the metabolic disorder is type 2 diabetes, and the therapeutic agent is chosen from metformin, insulin, glyburide, glipizide, glimepiride, repaglinide, nateglinide, thiazolidinediones, rosiglitazone, pioglitazone, sitagliptin, saxagliptin, linagliptin, exenatide, liraglutide, semaglutide, canagliflozin, dapagliflozin, and empagliflozin, or any combination thereof.

In one embodiment, the metabolic disorder is obesity, and the therapeutic agent is chosen from orlistat, phentermine, topiramate, bupropion, naltrexone, and liraglutide, or any combination thereof.

In one embodiment, the metabolic disorder is elevated triglyceride, and the therapeutic agent is chosen from rosuvastatin, simvastatin, atorvastatin, fenofibrate, gemfibrozil, fenofibric acid, niacin, and an omega-3 fatty acid, or any combination thereof.

In one embodiment, the metabolic disorder is lipodystrophy, and the therapeutic agent is chosen from tesamorelin, metformin, poly-L-lactic acid, calcium hydroxyapatite, polymethylmethacrylate, bovine collagens, human collagens, silicone, and hyaluronic acid, or any combination thereof.

In one embodiment, the metabolic disorder is liver inflammation, and the therapeutic agent is a hepatitis therapeutic or a hepatitis vaccine.

In one embodiment, the metabolic disorder is fatty liver disease include, and the subject is administered bariatric surgery and/or dietary intervention.

In one embodiment, the metabolic disorder is hypercholesterolemia, and the therapeutic agent is chosen from: atorvastatin, fluvastatin, lovastatin, pitavastatin, pravastatin, rosuvastatin calcium, simvastatin, cholestyramine, colesevelam, and colestipol, alirocumab, evolocumab, niaspan, niacor, fenofibrate, gemfibrozil, and bempedoic, or any combination thereof.

In one embodiment, the metabolic disorder is an elevated liver enzyme), and the therapeutic agent is chosen from coffee, folic acid, potassium, vitamin B6, a statin, and fiber, or any combination thereof.

In one embodiment, the metabolic disorder is nonalcoholic steatohepatitis (NASH) and the therapeutic agent is obeticholic acid, Selonsertib, Elafibranor, Cenicriviroc, GR_MD_02, MGL_3196, IMM124E, arachidyl amido cholanoic acid, GS0976, Emricasan, Volixibat, NGM282, GS9674, Tropifexor, MN_001, LMB763, B1_1467335, MSDC_0602, PF_05221304, DF102, Saroglitazar, BMS986036, Lanifibranor, Semaglutide, Nitazoxanide, GRI_0621, EYP001, VK2809, Nalmefene, LIK066, MT 3995, Elobixibat, Namodenoson, Foralumab, SAR425899, Sotagliflozin, EDP_305, Isosabutate, Gemcabene, TERN 101, KBP_042, PF_06865571, DUR928, PF_06835919, NGM313, BMS_986171, Namacizumab, CER_209, ND_L02_s0201, RTU_1096, DRX_065, IONIS_DGAT2Rx, INT_767, NC_001, Seladepar, PXL770, TERN_201, NV556, AZD2693, SP_1373, VK0214, Hepastem, TGFTX4, RLBN1127, GKT_137831, RYI_018, CB4209-CB4211, and JH_0920.

In one embodiment, the therapeutic agent that treats or inhibits the metabolic disorder is a melanocortin 4 receptor (MC4R) agonist.

In one embodiment, the MC4R agonist comprises a protein, a peptide, a nucleic acid molecule, or a small molecule.

In one embodiment, the protein is a peptide analog of MC4R.

In one embodiment, the peptide is setmelanotide.

In one embodiment, the MC4R agonist is a peptide comprising the amino acid sequence His-Phe-Arg-Trp.

In one embodiment, the small molecule is 1,2,3R,4-tetrahydroisoquinoline-3-carboxylic acid.

In one embodiment, the MC4R agonist is ALB-127158(a).

In one embodiment, the cardiovascular disease is high blood pressure, and the therapeutic agent is chosen from chlorthalidone, chlorothiazide, hydrochlorothiazide, indapamide, metolazone, acebutolol, atenolol, betaxolol, bisoprolol fumarate, carteolol hydrochloride, metoprolol tartrate, metoprolol succinate, nadolol, benazepril hydrochloride, captopril, enalapril maleate, fosinopril sodium, lisinopril, moexipril, perindopril, quinapril hydrochloride, ramipril, trandolapril, candesartan, eprosartan mesylate, irbesartan, losartan potassium, telmisartan, valsartan, amlodipine besylate, bepridil, diltiazem hydrochloride, felodipine, isradipine, nicardipine, nifedipine, nisoldipine, verapamil hydrochloride, doxazosin mesylate, prazosin hydrochloride, terazosin hydrochloride, methyldopa, carvedilol labetalol hydrochloride, alpha methyldopa, clonidine hydrochloride, guanabenz acetate, guanfacine hydrochloride, guanadrel, guanethidine monosulfate, reserpine, hydralazine hydrochloride, and minoxidil, or any combination thereof.

In one embodiment, the cardiovascular disease is cardiomyopathy, and the therapeutic agent is an ACE inhibitor, an angiotensin II receptor blocker, a beta blocker, a calcium channel blocker, digoxin, an antiarrhythmic, an aldosterone blocker, a diuretic, an anticoagulant, a blood thinner, and a corticosteroid.

In one embodiment, the cardiovascular disease is heart failure, and the therapeutic agent is an ACE inhibitor, an angiotensin-2 receptor blocker, a beta blocker, a mineralocorticoid receptor antagonist, a diuretic, ivabradine, sacubitril valsartan, hydralazine with nitrate, and digoxin.

The iRNA agent and an additional therapeutic agent and/or treatment may be administered at the same time and/or in the same combination, e.g., parenterally, or the additional therapeutic agent can be administered as part of a separate composition or at separate times and/or by another method known in the art or described herein.

### XII. Kits

In certain aspects, the instant disclosure provides kits that include a suitable container containing a pharmaceutical formulation of a siRNA compound, e.g., a double-stranded siRNA compound, or siRNA compound, (e.g., a precursor, e.g., a larger siRNA compound which can be processed into a siRNA compound, or a DNA which encodes an siRNA compound, e.g., a double-stranded siRNA compound, or ssiRNA compound, or precursor thereof).

Such kits include one or more dsRNA agent(s) and instructions for use, e.g., instructions for administering a prophylactically or therapeutically effective amount of a dsRNA agent(s). The dsRNA agent may be in a vial or a pre-filled syringe. The kits may optionally further comprise means for administering the dsRNA agent (e.g., an injection device, such as a pre-filled syringe), or means for measuring the inhibition of a metabolic disorder-associated target gene, e.g., INHBE, ACVR1C, PLIN1, PDE3B, or INHBC (e.g., means for measuring the inhibition of target gene mRNA, target gene protein, and/or target gebe activity). Such means for measuring the inhibition of target gene may comprise a means for obtaining a sample from a subject, such as, e.g., a plasma sample. The kits of the invention may optionally further comprise means for determining the therapeutically effective or prophylactically effective amount.

In certain embodiments the individual components of the pharmaceutical formulation may be provided in one container, e.g., a vial or a pre-filled syringe. Alternatively, it may be desirable to provide the components of the pharmaceutical formulation separately in two or mom containers, e.g., one container for a siRNA compound preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

This invention is further illustrated by the following examples which should not be construed as limiting. The entire contents of all references, patents and published patent applications cited throughout this application, as well as the informal Sequence Listing and Figures, are hereby incorporated herein by reference.

### EXAMPLES

### Example 1. Identification of Association of INHBE Loss-Of-Function with Waist-To-Hip Ratio in UK Biobank

Abdominal obesity is the most prevalent manifestation of metabolic syndrome (Després J. and Lemieux I. Nature 2006; 444:881-887) and is recognized as a contributor to cardiovascular disease and metabolic risk beyond body mass index (BMI) (Neeland U et al. Lancet Diabetes & Endocrinology 2019; 7(9):715-725). Waist-to-hip ratio adjusted for BMI (WHRadjBMI) reflects abdominal adiposity and correlates with direct imaging of abdominal fat. Mendelian randomization studies have shown a causal relationship between WHRadjBMI and risk of type 2 diabetes and coronary heart disease along with ischemic stroke, glycemic traits and circulating lipids (Emdin C A et al. JAMA 2017; 317(6):626-634; Dale C E et al. Circulation 2017; 135(24):2373-2388).

Rare genetic variants were tested for association with waist-to-hip ratio adjusted for BMI using exome sequencing data from the UK Biobank (UKBB). UKBB, a large long-term biobank study in the United Kingdom (UK) is investigating the respective contributions of genetic predisposition and environmental exposure (including nutrition, lifestyle, medications etc.) to the development of disease (see, e.g., www.ukbiobank.ac.uk). The study is following about 500,000 volunteers in the UK, enrolled at ages from 40 to 69. Initial enrollment took place over four years from 2006, and the volunteers will be followed for at least 30 years thereafter. A plethora of phenotypic data has been collected including anthropometric measurements such as waist and hip circumference. Recently, the exome sequencing data (or the portion of the genomes composed of exons) from about 450,000 participants in the study has been obtained.

These whole exome sequences were used to identify rare predicted loss-of-function (pLOF) variants (i.e., frameshift, stop gain, splice donor or splice acceptor variants) called as high confidence by LOFTEE. WHR adjBMI were calculated for participants using manual measurements for waist circumference, hip circumference, and body mass index (BMI) which were taken at their UKBB assessment. WHR was calculated as the ratio of these two measurements. Using these data, along with age at recruitment and sex, a linear model was built modeling WHR (WHR˜Age+Sex+BMI). WHR adjBMI was defined using the residuals from this model.

Gene-based collapsing tests (i.e., burden tests) were used to look for associations between rare (minor allele frequency ≤1%) pLOF variants and WHRadjBMI. Burden testing was performed in the unrelated White population (n=363,973) adjusting for age, sex and genetic ancestry via 12 principal components. INHBE pLOF associated with a 0.22 standard deviation decrease in WHRadjBMI (Table A). INHBE was tested for association with additional quantitative traits and we detected associations with birth weight, WHR (not adjusted for BMI), triglycerides and HDL cholesterol (Table A). INHBE pLOF also has a lower odds ratio for hypertension, coronary heart disease and T2D (Table B)

The most common INHBE pLOF variant in the UKBB exome-sequencing data was a splice acceptor variant (rs150777893) carried by 536 out of 620 pLOF carriers. Tested as a single variant, rs150777893 significantly associated with decreased WHRadj BMI (Table C).

TABLE A
Association of INHBE pLOF with WHRadj BMI and other traits

| | | | Effect | N carrier |
|---|---|---|---|---|
| | Variant set | pvalue | (SD) | measured |
| WaistHipRatioAdjBMI | INHBE pLOF | 4.76E−08 | −0.22 | 618 |
| EarlyLife_Birth_weight | INHBE pLOF | 7.01E−07 | 0.26 | 345 |
| WaistHipRatio | INHBE pLOF | 3.45E−05 | −0.13 | 619 |
| Blood_Biochemistry_Triglycerides | INHBE pLOF | 0.001 | −0.13 | 594 |
| Blood_Biochemistry_HDL_cholesterol | INHBE pLOF | 0.01 | 0.10 | 550 |

TABLE B
Association of INHBE pLOF with hypertension, heat disease and T2D

| | | Odds ratio | n carrier | n |
|---|---|---|---|---|
| phenotype | pvalue | (95% CI) | cases | expected |
| I10_essential_primary_hypertension | 0.10 | 0.86 | 186 | 202.31 |
| | | (0.71, 1.03) | | |
| I25_chronic_ischaemic_heart_disease | 0.21 | 0.83 | 56 | 61.31 |
| | | (0.63, 1.11) | | |
| E11_non_insulin_dependent_diabetes | 0.36 | 0.87 | 45 | 50.30 |
| | | (0.64, 1.18) | | |

TABLE C
Association of splice acceptor variant rs150777893 with WHRadjBMI

| | | | | | | Effect | | | | MAF | N white |
|---|---|---|---|---|---|---|---|---|---|---|---|
| | chrom | pos | ref | alt | pvalue | (SD) | rsid | gene | consequence | white | carriers |
| WHRAdjBMI | 12 | 57456093 | G | C | 3.75E−08 | −0.24 | rs150777893 | INHBE | splice | 0.07% | 539 |
| | | | | | | | | | acceptor | | |
| | | | | | | | | | variant | | |

### Example 2. iRNA Synthesis

### Source of Reagents

Where the source of a reagent is not specifically given herein, such reagent can be obtained from any supplier of reagents for molecular biology at a quality/purity standard for application in molecular biology.

siRNA Design

siRNAs targeting the inhibin subunit beta E gene (INHBE, human: NCBI refseqID NM_031479.5, NCBI Gene ID: 83729) were designed using custom R and Python scripts. The human NM 031479.5 mRNA has a length of 2460 bases.

Detailed lists of the unmodified INHBE sense and antisense strand nucleotide sequences are shown in Table 2.

Detailed lists of the modified INHBE sense and antisense strand nucleotide sequences are shown in Table 3.

siRNAs targeting the activin A receptor type 1C (ACVR1C) gene (ACVR1C, human: NCBI refseqID NM_145259.3, NCBI Gene ID: 130399) were designed using custom R and Python scripts. The human NM_145259.3 mRNA has a length of 8853 bases.

Detailed lists of the unmodified sense and antisense strand sequences of ACVR1C dsRNA agents comprising an unsaturated C22 hydrocarbon chain conjugated to position 6 on the sense strand, counting from the 5′-end of the sense strand are shown in Table 4.

Detailed lists of the modified sense and antisense strand sequences of ACVR1C dsRNA agents comprising an unsaturated C22 hydrocarbon chain conjugated to position 6 on the sense strand, counting from the 5′-end of the sense strand are shown in Table 5.

Detailed lists of the unmodified sense and antisense strand sequences of ACVR1C dsRNA agents comprising a GalNAc derivative targeting ligand are shown in Table 6.

Detailed lists of the modified sense and antisense strand sequences of ACVR1C dsRNA agents comprising a GalNAc derivative targeting ligand are shown in Table 7.

siRNAs targeting the perilipin-1 (PLIN1) gene (PLIN1, human: NCBI refseqID NM_002666.5, NCBI Gene ID: 5346) were designed using custom R and Python scripts. The human NM_002666.5 mRNA has a length of 2916 bases.

Detailed lists of the unmodified sense and antisense strand sequences of PLIN1 dsRNA agents comprising an unsaturated C22 hydrocarbon chain conjugated to position 6 on the sense strand, counting from the 5′-end of the sense strand are shown in Table 8.

Detailed lists of the modified sense and antisense strand sequences of PLIN1 dsRNA agents comprising an unsaturated C22 hydrocarbon chain conjugated to position 6 on the sense strand, counting from the 5′-end of the sense strand are shown in Table 9.

Detailed lists of the unmodified sense and antisense strand sequences of PLIN1 dsRNA agents comprising a GalNAc derivative targeting ligand are shown in Table 10.

Detailed lists of the modified sense and antisense strand sequences of PLIN1 dsRNA agents comprising a GalNAc derivative targeting ligand are shown in Table 11.

siRNAs targeting the phosphodiesterase 3B (PDE3B) gene (PDE3B, human: NCBI refseqID NM_000922.4, NCBI Gene ID: 5140) were designed using custom R and Python scripts. The human NM_000922.4 mRNA has a length of 5995 bases.

Detailed lists of the unmodified sense and antisense strand sequences of PDE3B dsRNA agents comprising an unsaturated C22 hydrocarbon chain conjugated to position 6 on the sense strand, counting from the 5′-end of the sense strand are shown in Table 12.

Detailed lists of the modified sense and antisense strand sequences of PDE3B dsRNA agents comprising an unsaturated C22 hydrocarbon chain conjugated to position 6 on the sense strand, counting from the 5′-end of the sense strand are shown in Table 13.

Detailed lists of the unmodified sense and antisense strand sequences of PDE3B dsRNA agents comprising a GalNAc derivative targeting ligand are shown in Table 14.

Detailed lists of the modified sense and antisense strand sequences of PDE3B dsRNA agents comprising a GalNAc derivative targeting ligand are shown in Table 15.

siRNAs targeting the inhibin subunit beta C (INHBC) gene (INHBC, human: NCBI refseqID NM_005538.4, NCBI Gene ID: 3626) were designed using custom R and Python scripts. The human NM_005538.4, mRNA has a length of 3202 bases.

Detailed lists of the unmodified sense and antisense strand sequences of INHBC dsRNA agents comprising a GalNAc derivative targeting ligand are shown in Table 16.

Detailed lists of the modified sense and antisense strand sequences of INHBC dsRNA agents comprising a GalNAc derivative targeting ligand are shown in Table 17.

It is to be understood that, throughout the application, a duplex name without a decimal is equivalent to a duplex name with a decimal which merely references the batch number of the duplex. For example, AD-959917 is equivalent to AD-959917.1.

siRNA Synthesis

siRNAs were designed, synthesized, and prepared using methods known in the art.

Briefly, siRNA sequences were synthesized on a 1 μmol scale using a Mermade 192 synthesizer (BioAutomation) with phosphoramidite chemistry on solid supports. The solid support was controlled pore glass (500-1000 Å) loaded with a custom GalNAc ligand (3′-GalNAc conjugates), universal solid support (AM Chemicals), or the first nucleotide of interest. Ancillary synthesis reagents and standard 2-cyanoethyl phosphoramidite monomers (2′-deoxy-2′-fluoro, 2′-O-methyl, RNA, DNA) were obtained from Thermo-Fisher (Milwaukee, WI), Hongene (China), or Chemgenes (Wilmington, MA, USA). Additional phosphoramidite monomers were procured from commercial suppliers, prepared in-house, or procured using custom synthesis from various CMOs. Phosphoramidites were prepared at a concentration of 100 mM in either acetonitrile or 9:1 acetonitrile:DMF and were coupled using 5-Ethylthio-1H-tetrazole (ETT, 0.25 M in acetonitrile) with a reaction time of 400 s. Phosphorothioate linkages were generated using a 100 mM solution of 3-((Dimethylamino-methylidene) amino)-3H-1,2,4-dithiazole-3-thione (DDTT, obtained from Chemgenes (Wilmington, MA, USA)) in anhydrous acetonitrile/pyridine (9:1 v/v). Oxidation time was 5 minutes. All sequences were synthesized with final removal of the DMT group (“DMT-Off”).

Upon completion of the solid phase synthesis, solid-supported oligoribonucleotides were treated with 300 μL of Methylamine (40% aqueous) at room temperature in 96 well plates for approximately 2 hours to afford cleavage from the solid support and subsequent removal of all additional base-labile protecting groups. For sequences containing any natural ribonucleotide linkages (2′-OH) protected with a tert-butyl dimethyl silyl (TBDMS) group, a second deprotection step was performed using TEA·3HF (triethylamine trihydrofluoride). To each oligonucleotide solution in aqueous methylamine was added 200 μL of dimethyl sulfoxide (DMSO) and 300 μL TEA·3HF and the solution was incubated for approximately 30 mins at 60° C. After incubation, the plate was allowed to come to room temperature and crude oligonucleotides were precipitated by the addition of 1 mL of 9:1 acetontrile:ethanol or 1:1 ethanol:isopropanol. The plates were then centrifuged at 4° C. for 45 mins and the supernatant carefully decanted with the aid of a multichannel pipette. The oligonucleotide pellet was resuspended in 20 mM NaOAc and subsequently desalted using a HiTrap size exclusion column (5 mL, GE Healthcare) on an Agilent LC system equipped with an autosampler, UV detector, conductivity meter, and fraction collector. Desalted samples were collected in 96 well plates and then analyzed by LC-MS and UV spectrometry to confirm identity and quantify the amount of material, respectively.

Duplexing of single strands was performed on a Tecan liquid handling robot. Sense and antisense single strands were combined in an equimolar ratio to a final concentration of 10 μM in 1×PBS in 96 well plates, the plate sealed, incubated at 100° C. for 10 minutes, and subsequently allowed to return slowly to room temperature over a period of 2-3 hours. The concentration and identity of each duplex was confirmed and then subsequently utilized for in vitro screening assays.

### Example 3. In Vitro Screening Methods

### Cell Culture and 96-Well Transfections

Hep3b cells (ATCC, Manassas, VA) were grown to near confluence at 37° C. in an atmosphere of 5% CO₂ in Eagle's Minimum Essential Medium (Gibco) supplemented with 10% FBS (ATCC) before being released from the plate by trypsinization. Transfection was carried out by adding 7.5 μl of Opti-MEM plus 0.3 μl of Lipofectamine RNAiMax per well (Invitrogen, Carlsbad CA. cat 13778-150) to 2.5 μl of each siRNA duplex to an individual well in a 384-well plate. The mixture was then incubated at room temperature for 15 minutes. Forty μl of complete growth media without antibiotic containing ˜1.5×10⁴ cells were then added to the siRNA mixture. Cells were incubated for 24 hours prior to RNA purification. Single dose experiments were performed at 10 nM, and 1 nM final duplex concentration.

Total RNA Isolation Using DYNABEADS mRNA Isolation Kit (Invitrogen™, Part #: 610-12)

Cells were lysed in 75 μl of Lysis/Binding Buffer containing 3 μL of beads per well and mixed for 10 minutes on an electrostatic shaker. The washing steps were automated on a Biotek EL406, using a magnetic plate support. Beads were washed (in 90 μL) once in Buffer A, once in Buffer B, and twice in Buffer E, with aspiration steps in between. Following a final aspiration, complete 10 μL RT mixture was added to each well, as described below.

cDNA Synthesis Using ABI High Capacity cDNA Reverse Transcription Kit (Applied Biosystems, Foster City, CA, Cat #4368813)

A master mix of 1 μl 10× Buffer, 0.4 μl 25×dNTPs, 1 μl Random primers, 0.50 Reverse

Transcriptase, 0.5 μl RNase inhibitor and 6.6 μl of H₂O per reaction were added per well. Plates were sealed, agitated for 10 minutes on an electrostatic shaker, and then incubated at 37 degrees C. for 2 hours. Following this, the plates were agitated at 80 degrees C. for 8 minutes.

### Real Time PCR

Two microlitre (μl) of cDNA were added to a master mix containing 0.5 μl of human GAPDH

TaqMan Probe (4326317E), 0.5 μl human INHBE, 2 μl nuclease-free water and 5 μl Lightcycler 480 probe master mix (Roche Cat #04887301001) per well in a 384 well plates (Roche cat #04887301001). Real time PCR was done in a LightCycler480 Real Time PCR system (Roche).

To calculate relative fold change, data were analyzed using the ΔΔCt method and normalized assays performed with cells transfected with 10 nM AD-1955, or mock transfected cells. IC₅₀s are calculated using a 4 parameter fit model using XLFit and normalized to cells transfected with AD-1955 or mock-transfected. The sense and antisense sequences of AD-1955 are: sense:

(SEQ ID NO: 126)
cuuAcGcuGAGuAcuucGAdTsdT
and
antisense
(SEQ ID NO: 127)
UCGAAGuACUcAGCGuAAGdTsdT
and
antisense
UCGAAGUACUcAGCGuAAGdTsdT.

Table 18 shows the results of a single dose screen in Hep3b cells transfected with the indicated agents from Tables 2 and 3.

TABLE 1
Abbreviations of nucleotide monomers used in nucleic acid sequence representation. It
will be understood that these monomers, when present in an oligonucleotide, are mutually linked by
5′-3′-phosphodiester bonds; and it is understood that when the nucleotide contains a 2′-fluoro
modification, then the fluoro replaces the hydroxy at that position in the parent nucleotide (i.e., it is a
2′-deoxy-2′-fluoronucleotide).

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide, modified or unmodified |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L10 | N-(cholesterylcarboxamidocaproyl)-4-hydroxyprolinol (Hyp-C6-Chol) |
| L96 | N-[tris(GalNAc-alkyl)-amidodecanoyl)]-4-hydroxyprolinol |
| | (Hyp-(GalNAc-alkyl)3) |
| | 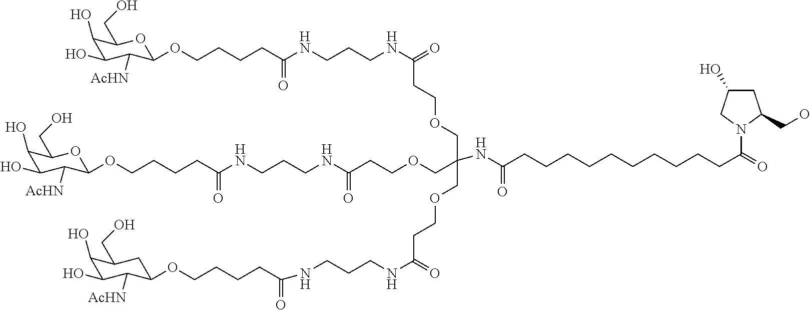 [SMILES: CC(=O)NC1C(OCCCCC(=O)NCCCNC(=O)CCOCC(COCCC(=O)NCCCNC(=O)CCCCOC2OC(CO)C(O)C(O)C2NC(C)=O)(COCCC(=O)NCCCNC(=O)CCCCOC2OC(CO)C(O)C(O)C2NC(C)=O)NC(=O)CCCCCCCCCCC(=O)N2C[C@H](O)C[C@H]2CO)CC(CO)C(O)C1O] |
| Y34 | 2-hydroxymethyl-tetrahydrofurane-4-methoxy-3-phosphate (abasic 2′-OMe |
| | furanose) |
| Y44 | inverted abasic DNA (2-hydroxymethyl-tetrahydrofurane-5-phosphate) |
| (Agn) | Adenosine-glycol nucleic acid (GNA) |
| (Cgn) | Cytidine-glycol nucleic acid (GNA) |
| (Ggn) | Guanosine-glycol nucleic acid (GNA) |
| (Tgn) | Thymidine-glycol nucleic acid (GNA) S-Isomer |
| P | Phosphate |
| VP | Vinyl-phosphonate |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythimidine-3′-phosphate |
| dTs | 2′-deoxythimidine-3′-phosphorothioate |
| dU | 2′-deoxyuridine |
| dUs | 2′-deoxyuridine-3′-phosphorothioate |
| (C2p) | cytidine-2′-phosphate |
| (G2p) | guanosine-2′-phosphate |
| (U2p) | uridine-2′-phosphate |
| (A2p) | adenosine-2′ -phosphate |
| (Chd) | 2′-O-hexadecyl-cytidine-3′-phosphate |
| (Ahd) | 2′-O-hexadecyl-adenosine-3′-phosphate |
| (Ghd) | 2′-O-hexadecyl-guanosine-3′-phosphate |
| (Uhd) | 2′-O-hexadecyl-uridine-3′-phosphate |
| Ada | 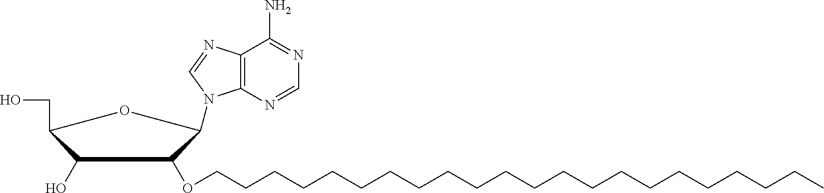 |
| | (2R,3S,4S,5R)-5-(6-amino-9H-purin-9-yl)-4-(docosyloxy)-2- |
| | (hydroxymethyl)tetrahydrofuran-3-ol |
| Cda | 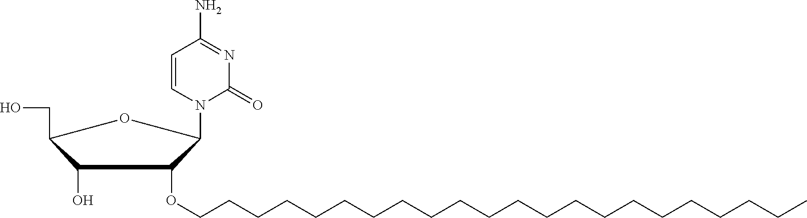 |
| | 4-amino-1-((2R,3R,4R,5R)-3-(docosyloxy)-4-hydroxy-5-(hydroxymethyl)tetrahydrofuran-2- |
| | yl)pyrimidin-2(1H)-one |
| Gda | 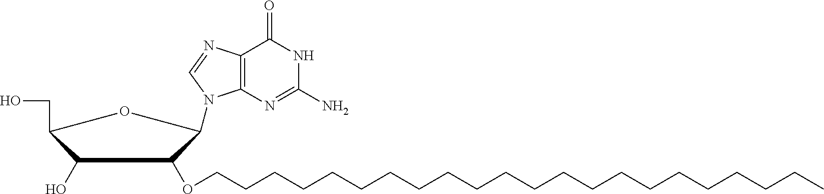 |
| | 2-amino-9-((2R,3S,4S,5R)-3-(docosyloxy)-4-hydroxy-5-(hydroxymethyl)tetrahydrofuran-2-yl)-1,9- |
| | dihydro-6H-purin-6-one |
| Uda | 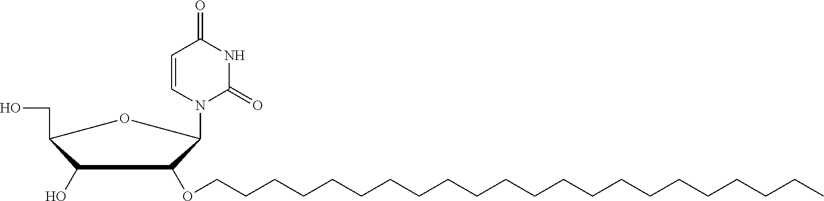 |
| | 1-((2R,3R,4R,5R)-3-(docosyloxy)-4-hydroxy-5-(hydroxymethyl)tetrahydrofuran-2- |
| | yl)pyrimidine-2,4(1H,3H)-dione |

TABLE 2
Unmodified Sense and Antisense Strand Sequences of INHBE dsRNA Agents

| | | SEQ | Range in | | SEQ |
|---|---|---|---|---|---|
| | | ID | INHBE mRNA | Antisense Sequence | ID |
| Duplex Name | Sense Sequence 5′ to 3′ | NO: | NM_031479.5 | 5′ to 3′ | NO: |
| AD-1656008 | UAGCCAGACAUGAGCUGUGAU | 128 | 1-23 | AUCACAGCUCAUGUCUGGCUACU | 262 |
| AD-1656026 | GAGGGUCAAGCACAGCUAUCU | 129 | 19-41 | AGAUAGCUGUGCUUGACCCUCAC | 263 |
| AD-1656043 | AUCCAUCAGAUGAUCUACUUU | 130 | 36-58 | AAAGUAGAUCAUCUGAUGGAUAG | 264 |
| AD-1656054 | GAUCUACUUUCAGCCUUCCUU | 131 | 47-69 | AAGGAAGGCUGAAAGUAGAUCAU | 265 |
| AD-1656074 | GAGUCCCAGACAAUAGAAGAU | 132 | 67-89 | AUCUUCUAUUGUCUGGGACUCAG | 266 |
| AD-1656086 | AUAGAAGACAGGUGGCUGUAU | 133 | 79-101 | AUACAGCCACCUGUCUUCUAUUG | 267 |
| AD-1656097 | GUGGCUGUACCCUUGGCCAAU | 134 | 90-112 | AUUGGCCAAGGGUACAGCCACCU | 268 |
| AD-1656108 | CUUGGCCAAGGGUAGGUGUGU | 135 | 101-123 | ACACACCUACCCUUGGCCAAGGG | 269 |
| AD-1656125 | GUGGCAGUGGUGUCUGCUGUU | 136 | 118-140 | AACAGCAGACACCACUGCCACAC | 270 |
| AD-1656139 | UGCUGUCACUGUGCCCUCAUU | 137 | 132-154 | AAUGAGGGCACAGUGACAGCAGA | 271 |
| AD-1656146 | AGCAAUCAGACUCAACAGACU | 138 | 160-182 | AGUCUGUUGAGUCUGAUUGCUGG | 272 |
| AD-1656164 | ACGGAGCAACUGCCAUCCGAU | 139 | 178-200 | AUCGGAUGGCAGUUGCUCCGUCU | 273 |
| AD-1656185 | GCUCCUGAACCAGGGCCAUUU | 140 | 199-221 | AAAUGGCCCUGGUUCAGGAGCCU | 274 |
| AD-1656196 | AGGGCCAUUCACCAGGAGCAU | 141 | 210-232 | AUGCUCCUGGUGAAUGGCCCUGG | 275 |
| AD-1656220 | GCUCCCUGAUGUCCAGCUCUU | 142 | 234-256 | AAGAGCUGGACAUCAGGGAGCCG | 276 |
| AD-1656233 | CAGCUCUGGCUGGUGCUGCUU | 143 | 247-269 | AAGCAGCACCAGCCAGAGCUGGA | 277 |
| AD-1656254 | UGGGCACUGGUGCGAGCACAU | 144 | 268-290 | AUGUGCUCGCACCAGUGCCCACA | 278 |
| AD-1656260 | AGGGUCUGUGUGUCCCUCCUU | 145 | 294-316 | AAGGAGGGACACACAGACCCUGU | 279 |
| AD-1656265 | CAAGCAGAACGAGCUCUGGUU | 146 | 337-359 | AACCAGAGCUCGUUCUGCUUGGG | 280 |
| AD-1656280 | CUGGUGCUGGAGCUAGCCAAU | 147 | 352-374 | AUUGGCUAGCUCCAGCACCAGAG | 281 |
| AD-1656292 | CUAGCCAAGCAGCAAAUCCUU | 148 | 364-386 | AAGGAUUUGCUGCUUGGCUAGCU | 282 |
| AD-1656307 | AUCCUGGAUGGGUUGCACCUU | 149 | 379-401 | AAGGUGCAACCCAUCCAGGAUUU | 283 |
| AD-1656319 | UUGCACCUGACCAGUCGUCCU | 150 | 391-413 | AGGACGACUGGUCAGGUGCAACC | 284 |
| AD-1656333 | UCGUCCCAGAAUAACUCAUCU | 151 | 405-427 | AGAUGAGUUAUUCUGGGACGACU | 285 |
| AD-1656360 | CCCUCCGGAGACUACAGCCAU | 152 | 452-474 | AUGGCUGUAGUCUCCGGAGGGCU | 286 |
| AD-1656372 | UACAGCCAGGGAGUGUGGCUU | 153 | 464-486 | AAGCCACACUCCCUGGCUGUAGU | 287 |
| AD-1656383 | AGUGUGGCUCCAGGGAAUGGU | 154 | 475-497 | ACCAUUCCCUGGAGCCACACUCC | 288 |
| AD-1656392 | CAUCAGCUUUGCUACUGUCAU | 155 | 504-526 | AUGACAGUAGCAAAGCUGAUGAC | 289 |
| AD-1656406 | CUGUCACAGACUCCACUUCAU | 68 | 518-540 | AUGAAGUGGAGUCUGUGACAGUA | 290 |
| AD-1656417 | UCCACUUCAGCCUACAGCUCU | 156 | 529-551 | AGAGCUGUAGGCUGAAGUGGAGU | 291 |
| AD-1656437 | CCUGCUCACUUUUCACCUGUU | 157 | 549-571 | AACAGGUGAAAAGUGAGCAGGGA | 292 |
| AD-1656449 | UCACCUGUCCACUCCUCGGUU | 158 | 561-583 | AACCGAGGAGUGGACAGGUGAAA | 293 |
| AD-1656468 | UCCCACCACCUGUACCAUGCU | 159 | 580-602 | AGCAUGGUACAGGUGGUGGGACC | 294 |
| AD-1656480 | UACCAUGCCCGCCUGUGGCUU | 160 | 592-614 | AAGCCACAGGCGGGCAUGGUACA | 295 |
| AD-1656492 | CCCUUCCUGGCACUCUUUGCU | 161 | 626-648 | AGCAAAGAGUGCCAGGAAGGGUG | 296 |
| AD-1656504 | CUCUUUGCUUGAGGAUCUUCU | 162 | 638-660 | AGAAGAUCCUCAAGCAAAGAGUG | 297 |
| AD-1656535 | ACUCUCCUGGCUGAGCACCAU | 163 | 694-716 | AUGGUGCUCAGCCAGGAGAGUGC | 298 |
| AD-1656547 | GAGCACCACAUCACCAACCUU | 164 | 706-728 | AAGGUUGGUGAUGUGGUGCUCAG | 299 |
| AD-1656559 | ACCAACCUGGGCUGGCAUACU | 165 | 718-740 | AGUAUGCCAGCCCAGGUUGGUGA | 300 |
| AD-1656570 | CUGGCAUACCUUAACUCUGCU | 166 | 729-751 | AGCAGAGUUAAGGUAUGCCAGCC | 301 |
| AD-1656587 | UGCCCUCUAGUGGCUUGAGGU | 167 | 746-768 | ACCUCAAGCCACUAGAGGGCAGA | 302 |
| AD-1656591 | AGAAGUCUGGUGUCCUGAAAU | 168 | 770-792 | AUUUCAGGACACCAGACUUCUCA | 303 |
| AD-1656602 | GUCCUGAAACUGCAACUAGAU | 169 | 781-803 | AUCUAGUUGCAGUUUCAGGACAC | 304 |
| AD-1656622 | ACAGCACAGUUACUGGACAAU | 170 | 821-843 | AUUGUCCAGUAACUGUGCUGUUG | 305 |
| AD-1656634 | CUGGACAACCGAGGCGGCUCU | 171 | 833-855 | AGAGCCGCCUCGGUUGUCCAGUA | 306 |
| AD-1656647 | GCGGCUCUUGGACACAGCAGU | 172 | 846-868 | ACUGCUGUGUCCAAGAGCCGCCU | 307 |
| AD-1656667 | GACACCAGCAGCCCUUCCUAU | 173 | 866-888 | AUAGGAAGGGCUGCUGGUGUCCU | 308 |
| AD-1656679 | CCUUCCUAGAGCUUAAGAUCU | 174 | 878-900 | AGAUCUUAAGCUCUAGGAAGGGC | 309 |
| AD-1656690 | CUUAAGAUCCGAGCCAAUGAU | 175 | 889-911 | AUCAUUGGCUCGGAUCUUAAGCU | 310 |
| AD-1656701 | AGCCAAUGAGCCUGGAGCAGU | 176 | 900-922 | ACUGCUCCAGGCUCAUUGGCUCG | 311 |
| AD-1656716 | AGGCGAGACCAUUACGUAGAU | 177 | 973-995 | AUCUACGUAAUGGUCUCGCCUGC | 312 |
| AD-1656728 | UACGUAGACUUCCAGGAACUU | 178 | 985-1007 | AAGUUCCUGGAAGUCUACGUAAU | 313 |
| AD-1656740 | CAGGAACUGGGAUGGCGGGAU | 179 | 997-1019 | AUCCCGCCAUCCCAGUUCCUGGA | 314 |
| AD-1656754 | GCGGGACUGGAUACUGCAGCU | 180 | 1011-1033 | AGCUGCAGUAUCCAGUCCCGCCA | 315 |
| AD-1656762 | UACCAGCUGAAUUACUGCAGU | 181 | 1039-1061 | ACUGCAGUAAUUCAGCUGGUACC | 316 |
| AD-1656775 | ACUGCAGUGGGCAGUGCCCUU | 182 | 1052-1074 | AAGGGCACUGCCCACUGCAGUAA | 317 |
| AD-1656792 | CAGGCAUUGCUGCCUCUUUCU | 183 | 1091-1113 | AGAAAGAGGCAGCAAUGCCUGGG | 318 |
| AD-1656808 | UUUCCAUUCUGCCGUCUUCAU | 184 | 1107-1129 | AUGAAGACGGCAGAAUGGAAAGA | 319 |
| AD-1656820 | CGUCUUCAGCCUCCUCAAAGU | 185 | 1119-1141 | ACUUUGAGGAGGCUGAAGACGGC | 320 |
| AD-1656832 | CCUCAAAGCCAACAAUCCUUU | 186 | 1131-1153 | AAAGGAUUGUUGGCUUUGAGGAG | 321 |
| AD-1656849 | CUUGGCCUGCCAGUACCUCCU | 187 | 1148-1170 | AGGAGGUACUGGCAGGCCAAGGA | 322 |
| AD-1656862 | UACCUCCUGUUGUGUCCCUAU | 188 | 1161-1183 | AUAGGGACACAACAGGAGGUACU | 323 |
| AD-1656873 | GUGUCCCUACUGCCCGAAGGU | 189 | 1172-1194 | ACCUUCGGGCAGUAGGGACACAA | 324 |
| AD-1656876 | CUCUCUCUCCUCUACCUGGAU | 190 | 1195-1217 | AUCCAGGUAGAGGAGAGAGAGGG | 325 |
| AD-1656888 | UACCUGGAUCAUAAUGGCAAU | 191 | 1207-1229 | AUUGCCAUUAUGAUCCAGGUAGA | 326 |
| AD-1656900 | AAUGGCAAUGUGGUCAAGACU | 192 | 1219-1241 | AGUCUUGACCACAUUGCCAUUAU | 327 |
| AD-1656915 | AAGACGGAUGUGCCAGAUAUU | 193 | 1234-1256 | AAUAUCUGGCACAUCCGUCUUGA | 328 |
| AD-1656926 | GCCAGAUAUGGUGGUGGAGGU | 194 | 1245-1267 | ACCUCCACCACCAUAUCUGGCAC | 329 |
| AD-1656942 | GAGGCCUGUGGCUGCAGCUAU | 195 | 1261-1283 | AUAGCUGCAGCCACAGGCCUCCA | 330 |
| AD-1656954 | UGCAGCUAGCAAGAGGACCUU | 196 | 1273-1295 | AAGGUCCUCUUGCUAGCUGCAGC | 331 |
| AD-1656958 | CUUUGGAGUGAAGAGACCAAU | 197 | 1297-1319 | AUUGGUCUCUUCACUCCAAAGCC | 332 |
| AD-1656969 | AGAGACCAAGAUGAAGUUUCU | 198 | 1308-1330 | AGAAACUUCAUCUUGGUCUCUUC | 333 |
| AD-1656996 | CAGGGCAUCUGUGACUGGAGU | 199 | 1335-1357 | ACUCCAGUCACAGAUGCCCUGUG | 334 |
| AD-1657013 | GAGGCAUCAGAUUCCUGAUCU | 200 | 1352-1374 | AGAUCAGGAAUCUGAUGCCUCCA | 335 |
| AD-1657022 | ACCCAACAACCACCUGGCAAU | 201 | 1381-1403 | AUUGCCAGGUGGUUGUUGGGUUG | 336 |
| AD-1657037 | GGCAAUAUGACUCACUUGACU | 202 | 1396-1418 | AGUCAAGUGAGUCAUAUUGCCAG | 337 |
| AD-1657045 | GACCCAAAUGGGCACUUUCUU | 203 | 1424-1446 | AAGAAAGUGCCCAUUUGGGUCCC | 338 |
| AD-1657058 | ACUUUCUUGUCUGAGACUCUU | 204 | 1437-1459 | AAGAGUCUCAGACAAGAAAGUGC | 339 |
| AD-1657069 | UGAGACUCUGGCUUAUUCCAU | 205 | 1448-1470 | AUGGAAUAAGCCAGAGUCUCAGA | 340 |
| AD-1657085 | UCCAGGUUGGCUGAUGUGUUU | 206 | 1464-1486 | AAACACAUCAGCCAACCUGGAAU | 341 |
| AD-1657099 | UGUGUUGGGAGAUGGGUAAAU | 207 | 1478-1500 | AUUUACCCAUCUCCCAACACAUC | 342 |
| AD-1657113 | GGUAAAGCGUUUCUUCUAAAU | 208 | 1492-1514 | AUUUAGAAGAAACGCUUUACCCA | 343 |
| AD-1657119 | UACCCAGAAAGCAUGAUUUCU | 209 | 1518-1540 | AGAAAUCAUGCUUUCUGGGUAGA | 344 |
| AD-1657133 | GAUUUCCUGCCCUAAGUCCUU | 210 | 1532-1554 | AAGGACUUAGGGCAGGAAAUCAU | 345 |
| AD-1657147 | AGUCCUGUGAGAAGAUGUCAU | 211 | 1546-1568 | AUGACAUCUUCUCACAGGACUUA | 346 |
| AD-1657164 | UCAGGGACUAGGGAGGGAGGU | 212 | 1563-1585 | ACCUCCCUCCCUAGUCCCUGACA | 347 |
| AD-1657185 | AAUUACUUAGCCUCUCCCAAU | 213 | 1600-1622 | AUUGGGAGAGGCUAAGUAAUUUU | 348 |
| AD-1657202 | CAAGAUGAGAAAGUCCUCAAU | 214 | 1617-1639 | AUUGAGGACUUUCUCAUCUUGGG | 349 |
| AD-1657211 | GGAGGAAGCAGAUAGAUGGUU | 215 | 1646-1668 | AACCAUCUAUCUGCUUCCUCCUC | 350 |
| AD-1657234 | GCAGGCUUGAAGCAGGGUAAU | 216 | 1669-1691 | AUUACCCUGCUUCAAGCCUGCUG | 351 |
| AD-1657245 | GCAGGGUAAGCAGGCUGGCCU | 217 | 1680-1702 | AGGCCAGCCUGCUUACCCUGCUU | 352 |
| AD-1657261 | GGCCCAGGGUAAGGGCUGUUU | 218 | 1696-1718 | AAACAGCCCUUACCCUGGGCCAG | 353 |
| AD-1657274 | GGCUGUUGAGGUACCUUAAGU | 219 | 1709-1731 | ACUUAAGGUACCUCAACAGCCCU | 354 |
| AD-1657286 | ACCUUAAGGGAAGGUCAAGAU | 220 | 1721-1743 | AUCUUGACCUUCCCUUAAGGUAC | 355 |
| AD-1657299 | GUCAAGAGGGAGAUGGGCAAU | 221 | 1734-1756 | AUUGCCCAUCUCCCUCUUGACCU | 356 |
| AD-1657322 | GCUGAGGGAGGAUGCUUAGGU | 222 | 1757-1779 | ACCUAAGCAUCCUCCCUCAGCGC | 357 |
| AD-1657324 | AGAAACAGGAGUCAGGAAAAU | 223 | 1785-1807 | AUUUUCCUGACUCCUGUUUCUGG | 358 |
| AD-1657335 | UCAGGAAAAUGAGGCACUAAU | 224 | 1796-1818 | AUUAGUGCCUCAUUUUCCUGACU | 359 |
| AD-1657347 | GGCACUAAGCCUAAGAAGUUU | 225 | 1808-1830 | AAACUUCUUAGGCUUAGUGCCUC | 360 |
| AD-1657359 | AAGAAGUUCCCUGGUUUUUCU | 226 | 1820-1842 | AGAAAAACCAGGGAACUUCUUAG | 361 |
| AD-1657374 | CACUGGGAGACAAGCAUUUAU | 227 | 1855-1877 | AUAAAUGCUUGUCUCCCAGUGGG | 362 |
| AD-1657385 | AAGCAUUUAUACUUUCUUUCU | 228 | 1866-1888 | AGAAAGAAAGUAUAAAUGCUUGU | 363 |
| AD-1657395 | UUUUGAGAUCGAGUCUCGCUU | 229 | 1899-1921 | AAGCGAGACUCGAUCUCAAAAAA | 364 |
| AD-1657410 | UCUCGCUCUGUCACCAGGCUU | 230 | 1912-1934 | AAGCCUGGUGACAGAGCGAGACU | 365 |
| AD-1657431 | AGUGCAGUGACACGAUCUUGU | 231 | 1934-1956 | ACAAGAUCGUGUCACUGCACUCC | 366 |
| AD-1657446 | GCUCACUGCAACCUCCGUCUU | 232 | 1954-1976 | AAGACGGAGGUUGCAGUGAGCCA | 367 |
| AD-1657457 | CCUCCGUCUCCUGGGUUCAAU | 233 | 1965-1987 | AUUGAACCCAGGAGACGGAGGUU | 368 |
| AD-1657463 | UUCUGCCUCAGCCUCCCGAGU | 234 | 1992-2014 | ACUCGGGAGGCUGAGGCAGAAGA | 369 |
| AD-1657475 | UGGGUUCAAGUGAUUCUUCUU | 235 | 1976-1998 | AAGAAGAAUCACUUGAACCCAGG | 370 |
| AD-1657503 | GAGCAGCUGGGAUUACAGGCU | 236 | 2009-2031 | AGCCUGUAAUCCCAGCUGCUCGG | 371 |
| AD-1657520 | CGCCCACUAAUUUUUGUAUUU | 237 | 2028-2050 | AAAUACAAAAAUUAGUGGGCGCC | 372 |
| AD-1657529 | UGUAUUCUUAGUAGAAACGAU | 238 | 2042-2064 | AUCGUUUCUACUAAGAAUACAAA | 373 |
| AD-1657540 | UAGAAACGAGGUUUCAACAUU | 239 | 2053-2075 | AAUGUUGAAACCUCGUUUCUACU | 374 |
| AD-1657552 | UUCAACAUGUUGGCCAGGAUU | 240 | 2065-2087 | AAUCCUGGCCAACAUGUUGAAAC | 375 |
| AD-1657564 | CCAGGAUGGUCUCAAUCUCUU | 241 | 2078-2100 | AAGAGAUUGAGACCAUCCUGGCC | 376 |
| AD-1657575 | UCAAUCUCUUGACCUCUUGAU | 242 | 2089-2111 | AUCAAGAGGUCAAGAGAUUGAGA | 377 |
| AD-1657586 | ACCUCUUGAUCCACCCGACUU | 243 | 2100-2122 | AAGUCGGGUGGAUCAAGAGGUCA | 378 |
| AD-1657600 | CCGACUUGGCCUCCCGAAGUU | 244 | 2114-2136 | AACUUCGGGAGGCCAAGUCGGGU | 379 |
| AD-1657615 | GAAGUGAUGAGAUUAUAGGCU | 245 | 2129-2151 | AGCCUAUAAUCUCAUCACUUCGG | 380 |
| AD-1657641 | CCUGGCUUAUACUUUCUUAAU | 246 | 2162-2184 | AUUAAGAAAGUAUAAGCCAGGCG | 381 |
| AD-1657653 | GAGAAAGAAAAUCAACAAAUU | 247 | 2189-2211 | AAUUUGUUGAUUUUCUUUCUCCU | 382 |
| AD-1657674 | UGAGUCAUAAAGAAGGGUUAU | 248 | 2210-2232 | AUAACCCUUCUUUAUGACUCACA | 383 |
| AD-1657687 | AGGGUUAGGGUGAUGGUCCAU | 249 | 2223-2245 | AUGGACCAUCACCCUAACCCUUC | 384 |
| AD-1657704 | CCAGAGCAACAGUUCUUCAAU | 250 | 2240-2262 | AUUGAAGAACUGUUGCUCUGGAC | 385 |
| AD-1657716 | UUCUUCAAGUGUACUCUGUAU | 251 | 2252-2274 | AUACAGAGUACACUUGAAGAACU | 386 |
| AD-1657727 | UACUCUGUAGGCUUCUGGGAU | 252 | 2263-2285 | AUCCCAGAAGCCUACAGAGUACA | 387 |
| AD-1657741 | CUGGGAGGUCCCUUUUCAGGU | 253 | 2277-2299 | ACCUGAAAAGGGACCUCCCAGAA | 388 |
| AD-1657744 | GUCCACAAAGUCAAAGCUAUU | 254 | 2300-2322 | AAUAGCUUUGACUUUGUGGACAC | 389 |
| AD-1657760 | CUAACAUGUUAUUUGCCUUUU | 255 | 2333-2355 | AAAAGGCAAAUAACAUGUUAGUA | 390 |
| AD-1657776 | CUUUUGAAUUCUCAUUAUCUU | 256 | 2349-2371 | AAGAUAAUGAGAAUUCAAAAGGC | 391 |
| AD-1657793 | GUAUUGUGGAGUUUUCCAGAU | 257 | 2376-2398 | AUCUGGAAAACUCCACAAUACAA | 392 |
| AD-1657811 | GAGGCCGUGUGACAUGUGAUU | 258 | 2394-2416 | AAUCACAUGUCACACGGCCUCUG | 393 |
| AD-1657822 | ACAUGUGAUUACAUCAUCUUU | 259 | 2405-2427 | AAAGAUGAUGUAAUCACAUGUCA | 394 |
| AD-1657834 | AUCAUCUUUCUGACAUCAUUU | 260 | 2417-2439 | AAAUGAUGUCAGAAAGAUGAUGU | 395 |
| AD-1657845 | GACAUCAUUGUUAAUGGAAUU | 261 | 2428-2450 | AAUUCCAUUAACAAUGAUGUCAG | 396 |

TABLE 3
Modified Sense and Antisense Strand Sequences of INHBE dsRNA Agents

| | | SEQ | | SEQ | | SEQ |
|---|---|---|---|---|---|---|
| | | ID | | ID | | ID |
| Duplex Name | Sense Sequence 5′ to 3′ | NO: | Antisense Sequence 5′ to 3′ | NO: | mRNA target sequence | NO: |
| AD-1656008 | usasgccaGfaCfAfUfgagcugugauL96 | 397 | asUfscacAfgCfUfcaugUfcUfggcuascsu | 532 | AGTAGCCAGACATGAGCTGTGAG | 667 |
| AD-1656026 | gsasggguCfaAfGfCfacagcuaucuL96 | 398 | asGfsauaGfcUfGfugcuUfgAfcccucsasc | 533 | GTGAGGGTCAAGCACAGCTATCC | 668 |
| AD-1656043 | asusccauCfaGfAfUfgaucuacuuuL96 | 399 | asAfsaguAfgAfUfcaucUfgAfuggausasg | 534 | CTATCCATCAGATGATCTACTTT | 669 |
| AD-1656054 | gsasucuaCfuUfUfCfagccuuccuuL96 | 400 | asAfsggaAfgGfCfugaaAfgUfagaucsasu | 535 | ATGATCTACTTTCAGCCTTCCTG | 670 |
| AD-1656074 | gsasguccCfaGfAfCfaauagaagauL96 | 401 | asUfscuuCfuAfUfugucUfgGfgacucsasg | 536 | CTGAGTCCCAGACAATAGAAGAC | 671 |
| AD-1656086 | asusagaaGfaCfAfGfguggcuguauL96 | 402 | asUfsacaGfcCfAfccugUfcUfucuaususg | 537 | CAATAGAAGACAGGTGGCTGTAC | 672 |
| AD-1656097 | gsusggcuGfuAfCfCfcuuggccaauL96 | 403 | asUfsuggCfcAfAfggguAfcAfgccacscsu | 538 | AGGTGGCTGTACCCTTGGCCAAG | 673 |
| AD-1656108 | csusuggcCfaAfGfGfguagguguguL96 | 404 | asCfsacaCfcUfAfcccuUfgGfccaagsgsg | 539 | CCCTTGGCCAAGGGTAGGTGTGG | 674 |
| AD-1656125 | gsusggcaGfuGfGfUfgucugcuguuL96 | 405 | asAfscagCfaGfAfcaccAfcUfgccacsasc | 540 | GTGTGGCAGTGGTGTCTGCTGTC | 675 |
| AD-1656139 | usgscuguCfaCfUfGfugcccucauuL96 | 406 | asAfsugaGfgGfCfacagUfgAfcagcasgsa | 541 | TCTGCTGTCACTGTGCCCTCATT | 676 |
| AD-1656146 | asgscaauCfaGfAfCfucaacagacuL96 | 407 | asGfsucuGfuUfGfagucUfgAfuugcusgsg | 542 | CCAGCAATCAGACTCAACAGACG | 677 |
| AD-1656164 | ascsggagCfaAfCfUfgccauccgauL96 | 408 | asUfscggAfuGfGfcaguUfgCfuccguscsu | 543 | AGACGGAGCAACTGCCATCCGAG | 678 |
| AD-1656185 | gscsuccuGfaAfCfCfagggccauuuL96 | 409 | asAfsaugGfcCfCfugguUfcAfggagescsu | 544 | AGGCTCCTGAACCAGGGCCATTC | 679 |
| AD-1656196 | asgsggccAfuUfCfAfccaggagcauL96 | 410 | asUfsgcuCfcUfGfgugaAfuGfgcccusgsg | 545 | CCAGGGCCATTCACCAGGAGCAT | 680 |
| AD-1656220 | gscsucccUfgAfUfGfuccagcucuuL96 | 411 | asAfsgagCfuGfGfacauCfaGfggagcscsg | 546 | CGGCTCCCTGATGTCCAGCTCTG | 681 |
| AD-1656233 | csasgcucUfgGfCfUfggugcugcuuL96 | 412 | asAfsgcaGfcAfCfcagcCfaGfagcugsgsa | 547 | TCCAGCTCTGGCTGGTGCTGCTG | 682 |
| AD-1656254 | usgsggcaCfuGfGfUfgcgagcacauL96 | 413 | asUfsgugCfuCfGfcaccAfgUfgcccascsa | 548 | TGTGGGCACTGGTGCGAGCACAG | 683 |
| AD-1656260 | asgsggucUfgUfGfUfgucccuccuuL96 | 414 | asAfsggaGfgGfAfcacaCfaGfacccusgsu | 549 | ACAGGGTCTGTGTGTCCCTCCTG | 684 |
| AD-1656265 | csasagcaGfaAfCfGfagcucugguuL96 | 415 | asAfsccaGfaGfCfucguUfcUfgcuugsgsg | 550 | CCCAAGCAGAACGAGCTCTGGTG | 685 |
| AD-1656280 | csusggugCfuGfGfAfgcuagccaauL96 | 416 | asUfsuggCfuAfGfcuccAfgCfaccagsasg | 551 | CTCTGGTGCTGGAGCTAGCCAAG | 686 |
| AD-1656292 | csusagccAfaGfCfAfgcaaauccuuL96 | 417 | asAfsggaUfuUfGfcugcUfuGfgcuagscsu | 552 | AGCTAGCCAAGCAGCAAATCCTG | 687 |
| AD-1656307 | asusccugGfaUfGfGfguugcaccuuL96 | 418 | asAfsgguGfcAfAfcccaUfcCfaggaususu | 553 | AAATCCTGGATGGGTTGCACCTG | 688 |
| AD-1656319 | ususgcacCfuGfAfCfcagucguccuL96 | 419 | asGfsgacGfaCfUfggucAfgGfugcaascsc | 554 | GGTTGCACCTGACCAGTCGTCCC | 689 |
| AD-1656333 | uscsguccCfaGfAfAfuaacucaucuL96 | 420 | asGfsaugAfgUfUfauucUfgGfgacgascsu | 555 | AGTCGTCCCAGAATAACTCATCC | 690 |
| AD-1656360 | cscscuccGfgAfGfAfcuacagccauL96 | 421 | asUfsggcUfgUfAfgucuCfcGfgagggscsu | 556 | AGCCCTCCGGAGACTACAGCCAG | 691 |
| AD-1656372 | usascagcCfaGfGfGfaguguggcuuL96 | 422 | asAfsgccAfcAfCfucccUfgGfcuguasgsu | 557 | ACTACAGCCAGGGAGTGTGGCTC | 692 |
| AD-1656383 | asgsugugGfcUfCfCfagggaaugguL96 | 423 | asCfscauUfcCfCfuggaGfcCfacacuscsc | 558 | GGAGTGTGGCTCCAGGGAATGGG | 693 |
| AD-1656392 | csasucagCfuUfUfGfcuacugucauL96 | 424 | asUfsgacAfgUfAfgcaaAfgCfugaugsasc | 559 | GTCATCAGCTTTGCTACTGTCAC | 694 |
| AD-1656406 | csusgucaCfaGfAfCfuccacuucauL96 | 425 | asUfsgaaGfuGfGfagucUfgUfgacagsusa | 560 | TACTGTCACAGACTCCACTTCAG | 695 |
| AD-1656417 | uscscacuUfcAfGfCfcuacagcucuL96 | 426 | asGfsagcUfgUfAfggcuGfaAfguggasgsu | 561 | ACTCCACTTCAGCCTACAGCTCC | 696 |
| AD-1656437 | cscsugcuCfaCfUfUfuucaccuguuL96 | 427 | asAfscagGfuGfAfaaagUfgAfgcaggsgsa | 562 | TCCCTGCTCACTTTTCACCTGTC | 697 |
| AD-1656449 | uscsaccuGfuCfCfAfcuccucgguuL96 | 428 | asAfsccgAfgGfAfguggAfcAfggugasasa | 563 | TTTCACCTGTCCACTCCTCGGTC | 698 |
| AD-1656468 | uscsccacCfaCfCfUfguaccaugcuL96 | 429 | asGfscauGfgUfAfcaggUfgGfugggascsc | 564 | GGTCCCACCACCTGTACCATGCC | 699 |
| AD-1656480 | usasccauGfcCfCfGfccuguggcuuL96 | 430 | asAfsgccAfcAfGfgcggGfcAfugguascsa | 565 | TGTACCATGCCCGCCTGTGGCTG | 700 |
| AD-1656492 | cscscuucCfuGfGfCfacucuuugcuL96 | 431 | asGfscaaAfgAfGfugccAfgGfaagggsusg | 566 | CACCCTTCCTGGCACTCTTTGCT | 701 |
| AD-1656504 | csuscuuuGfcUfUfGfaggaucuucuL96 | 432 | asGfsaagAfuCfCfucaaGfcAfaagagsusg | 567 | CACTCTTTGCTTGAGGATCTTCC | 702 |
| AD-1656535 | ascsucucCfuGfGfCfugagcaccauL96 | 433 | asUfsgguGfcUfCfagccAfgGfagagusgsc | 568 | GCACTCTCCTGGCTGAGCACCAC | 703 |
| AD-1656547 | gsasgcacCfaCfAfUfcaccaaccuuL96 | 434 | asAfsgguUfgGfUfgaugUfgGfugcucsasg | 569 | CTGAGCACCACATCACCAACCTG | 704 |
| AD-1656559 | ascscaacCfuGfGfGfcuggcauacuL96 | 435 | asGfsuauGfcCfAfgcccAfgGfuuggusgsa | 570 | TCACCAACCTGGGCTGGCATACC | 705 |
| AD-1656570 | csusggcaUfaCfCfUfuaacucugcuL96 | 436 | asGfscagAfgUfUfaaggUfaUfgccagscsc | 571 | GGCTGGCATACCTTAACTCTGCC | 706 |
| AD-1656587 | usgscccuCfuAfGfUfggcuugagguL96 | 437 | asCfscucAfaGfCfcacuAfgAfgggcasgsa | 572 | TCTGCCCTCTAGTGGCTTGAGGG | 707 |
| AD-1656591 | asgsaaguCfuGfGfUfguccugaaauL96 | 438 | asUfsuucAfgGfAfcaccAfgAfcuucuscsa | 573 | TGAGAAGTCTGGTGTCCTGAAAC | 708 |
| AD-1656602 | gsusccugAfaAfCfUfgcaacuagauL96 | 439 | asUfscuaGfuUfGfcaguUfuCfaggacsasc | 574 | GTGTCCTGAAACTGCAACTAGAC | 709 |
| AD-1656622 | ascsagcaCfaGfUfUfacuggacaauL96 | 440 | asUfsuguCfcAfGfuaacUfgUfgcugususg | 575 | CAACAGCACAGTTACTGGACAAC | 710 |
| AD-1656634 | csusggacAfaCfCfGfaggcggcucuL96 | 441 | asGfsagcCfgCfCfucggUfuGfuccagsusa | 576 | TACTGGACAACCGAGGCGGCTCT | 711 |
| AD-1656647 | gscsggcuCfuUfGfGfacacagcaguL96 | 442 | asCfsugcUfgUfGfuccaAfgAfgccgcscsu | 577 | AGGCGGCTCTTGGACACAGCAGG | 712 |
| AD-1656667 | gsascaccAfgCfAfGfcccuuccuauL96 | 443 | asUfsaggAfaGfGfgcugCfuGfgugucscsu | 578 | AGGACACCAGCAGCCCTTCCTAG | 713 |
| AD-1656679 | cscsuuccUfaGfAfGfcuuaagaucuL96 | 444 | asGfsaucUfuAfAfgcucUfaGfgaaggsgsc | 579 | GCCCTTCCTAGAGCTTAAGATCC | 714 |
| AD-1656690 | csusuaagAfuCfCfGfagccaaugauL96 | 445 | asUfscauUfgGfCfucggAfuCfuuaagscsu | 580 | AGCTTAAGATCCGAGCCAATGAG | 715 |
| AD-1656701 | asgsccaaUfgAfGfCfcuggagcaguL96 | 446 | asCfsugcUfcCfAfggcuCfaUfuggcuscsg | 581 | CGAGCCAATGAGCCTGGAGCAGG | 716 |
| AD-1656716 | asgsgcgaGfaCfCfAfuuacguagauL96 | 447 | asUfscuaCfgUfAfauggUfcUfcgccusgsc | 582 | GCAGGCGAGACCATTACGTAGAC | 717 |
| AD-1656728 | usascguaGfaCfUfUfccaggaacuuL96 | 448 | asAfsguuCfcUfGfgaagUfcUfacguasasu | 583 | ATTACGTAGACTTCCAGGAACTG | 718 |
| AD-1656740 | csasggaaCfuGfGfGfauggcgggauL96 | 449 | asUfscccGfcCfAfucccAfgUfuccugsgsa | 584 | TCCAGGAACTGGGATGGCGGGAC | 719 |
| AD-1656754 | gscsgggaCfuGfGfAfuacugcagcuL96 | 450 | asGfscugCfaGfUfauccAfgUfcccgcscsa | 585 | TGGCGGGACTGGATACTGCAGCC | 720 |
| AD-1656762 | usasccagCfuGfAfAfuuacugcaguL96 | 451 | asCfsugcAfgUfAfauucAfgCfugguascsc | 586 | GGTACCAGCTGAATTACTGCAGT | 721 |
| AD-1656775 | ascsugcaGfuGfGfGfcagugcccuuL96 | 452 | asAfsgggCfaCfUfgcccAfcUfgcagusasa | 587 | TTACTGCAGTGGGCAGTGCCCTC | 722 |
| AD-1656792 | csasggcaUfuGfCfUfgccucuuucuL96 | 453 | asGfsaaaGfaGfGfcagcAfaUfgccugsgsg | 588 | CCCAGGCATTGCTGCCTCTTTCC | 723 |
| AD-1656808 | ususuccaUfuCfUfGfccgucuucauL96 | 454 | asUfsgaaGfaCfGfgcagAfaUfggaaasgsa | 589 | TCTTTCCATTCTGCCGTCTTCAG | 724 |
| AD-1656820 | csgsucuuCfaGfCfCfuccucaaaguL96 | 455 | asCfsuuuGfaGfGfaggcUfgAfagacgsgsc | 590 | GCCGTCTTCAGCCTCCTCAAAGC | 725 |
| AD-1656832 | cscsucaaAfgCfCfAfacaauccuuuL96 | 456 | asAfsaggAfuUfGfuuggCfuUfugaggsasg | 591 | CTCCTCAAAGCCAACAATCCTTG | 726 |
| AD-1656849 | csusuggcCfuGfCfCfaguaccuccuL96 | 457 | asGfsgagGfuAfCfuggcAfgGfccaagsgsa | 592 | TCCTTGGCCTGCCAGTACCTCCT | 727 |
| AD-1656862 | usasccucCfuGfUfUfgugucccuauL96 | 458 | asUfsaggGfaCfAfcaacAfgGfagguascsu | 593 | AGTACCTCCTGTTGTGTCCCTAC | 728 |
| AD-1656873 | gsusguccCfuAfCfUfgcccgaagguL96 | 459 | asCfscuuCfgGfGfcaguAfgGfgacacsasa | 594 | TTGTGTCCCTACTGCCCGAAGGC | 729 |
| AD-1656876 | csuscucuCfuCfCfUfcuaccuggauL96 | 460 | asUfsccaGfgUfAfgaggAfgAfgagagsgsg | 595 | CCCTCTCTCTCCTCTACCTGGAT | 730 |
| AD-1656888 | usasccugGfaUfCfAfuaauggcaauL96 | 461 | asUfsugcCfaUfUfaugaUfcCfagguasgsa | 596 | TCTACCTGGATCATAATGGCAAT | 731 |
| AD-1656900 | asasuggcAfaUfGfUfggucaagacuL96 | 462 | asGfsucuUfgAfCfcacaUfuGfccauusasu | 597 | ATAATGGCAATGTGGTCAAGACG | 732 |
| AD-1656915 | asasgacgGfaUfGfUfgccagauauuL96 | 463 | asAfsuauCfuGfGfcacaUfcCfgucuusgsa | 598 | TCAAGACGGATGTGCCAGATATG | 733 |
| AD-1656926 | gscscagaUfaUfGfGfugguggagguL96 | 464 | asCfscucCfaCfCfaccaUfaUfcuggcsasc | 599 | GTGCCAGATATGGTGGTGGAGGC | 734 |
| AD-1656942 | gsasggccUfgUfGfGfcugcagcuauL96 | 465 | asUfsagcUfgCfAfgccaCfaGfgccucscsa | 600 | TGGAGGCCTGTGGCTGCAGCTAG | 735 |
| AD-1656954 | usgscagcUfaGfCfAfagaggaccuuL96 | 466 | asAfsgguCfcUfCfuugcUfaGfcugcasgsc | 601 | GCTGCAGCTAGCAAGAGGACCTG | 736 |
| AD-1656958 | csusuuggAfgUfGfAfagagaccaauL96 | 467 | asUfsuggUfcUfCfuucaCfuCfcaaagscsc | 602 | GGCTTTGGAGTGAAGAGACCAAG | 737 |
| AD-1656969 | asgsagacCfaAfGfAfugaaguuucuL96 | 468 | asGfsaaaCfuUfCfaucuUfgGfucucususc | 603 | GAAGAGACCAAGATGAAGTTTCC | 738 |
| AD-1656996 | csasgggcAfuCfUfGfugacuggaguL96 | 469 | asCfsuccAfgUfCfacagAfuGfcccugsusg | 604 | CACAGGGCATCTGTGACTGGAGG | 739 |
| AD-1657013 | gsasggcaUfcAfGfAfuuccugaucuL96 | 470 | asGfsaucAfgGfAfaucuGfaUfgccucscsa | 605 | TGGAGGCATCAGATTCCTGATCC | 740 |
| AD-1657022 | ascsccaaCfaAfCfCfaccuggcaauL96 | 471 | asUfsugcCfaGfGfugguUfgUfugggususg | 606 | CAACCCAACAACCACCTGGCAAT | 741 |
| AD-1657037 | gsgscaauAfuGfAfCfucacuugacuL96 | 472 | asGfsucaAfgUfGfagucAfuAfuugccsasg | 607 | CTGGCAATATGACTCACTTGACC | 742 |
| AD-1657045 | gsascccaAfaUfGfGfgcacuuucuuL96 | 473 | asAfsgaaAfgUfGfcccaUfuUfgggucscsc | 608 | GGGACCCAAATGGGCACTTTCTT | 743 |
| AD-1657058 | ascsuuucUfuGfUfCfugagacucuuL96 | 474 | asAfsgagUfcUfCfagacAfaGfaaagusgsc | 609 | GCACTTTCTTGTCTGAGACTCTG | 744 |
| AD-1657069 | usgsagacUfcUfGfGfcuuauuccauL96 | 475 | asUfsggaAfuAfAfgccaGfaGfucucasgsa | 610 | TCTGAGACTCTGGCTTATTCCAG | 745 |
| AD-1657085 | uscscaggUfuGfGfCfugauguguuuL96 | 476 | asAfsacaCfaUfCfagccAfaCfcuggasasu | 611 | ATTCCAGGTTGGCTGATGTGTTG | 746 |
| AD-1657099 | usgsuguuGfgGfAfGfauggguaaauL96 | 477 | asUfsuuaCfcCfAfucucCfcAfacacasusc | 612 | GATGTGTTGGGAGATGGGTAAAG | 747 |
| AD-1657113 | gsgsuaaaGfcGfUfUfucuucuaaauL96 | 478 | asUfsuuaGfaAfGfaaacGfcUfuuaccscsa | 613 | TGGGTAAAGCGTTTCTTCTAAAG | 748 |
| AD-1657119 | usascccaGfaAfAfGfcaugauuucuL96 | 479 | asGfsaaaUfcAfUfgcuuUfcUfggguasgsa | 614 | TCTACCCAGAAAGCATGATTTCC | 749 |
| AD-1657133 | gsasuuucCfuGfCfCfcuaaguccuuL96 | 480 | asAfsggaCfuUfAfgggcAfgGfaaaucsasu | 615 | ATGATTTCCTGCCCTAAGTCCTG | 750 |
| AD-1657147 | asgsuccuGfuGfAfGfaagaugucauL96 | 481 | asUfsgacAfuCfUfucucAfcAfggacususa | 616 | TAAGTCCTGTGAGAAGATGTCAG | 751 |
| AD-1657164 | uscsagggAfcUfAfGfggagggagguL96 | 482 | asCfscucCfcUfCfccuaGfuCfccugascsa | 617 | TGTCAGGGACTAGGGAGGGAGGG | 752 |
| AD-1657185 | asasuuacUfuAfGfCfcucucccaauL96 | 483 | asUfsuggGfaGfAfggcuAfaGfuaauususu | 618 | AAAATTACTTAGCCTCTCCCAAG | 753 |
| AD-1657202 | csasagauGfaGfAfAfaguccucaauL96 | 484 | asUfsugaGfgAfCfuuucUfcAfucuugsgsg | 619 | CCCAAGATGAGAAAGTCCTCAAG | 754 |
| AD-1657211 | gsgsaggaAfgCfAfGfauagaugguuL96 | 485 | asAfsccaUfcUfAfucugCfuUfccuccsusc | 620 | GAGGAGGAAGCAGATAGATGGTC | 755 |
| AD-1657234 | gscsaggcUfuGfAfAfgcaggguaauL96 | 486 | asUfsuacCfcUfGfcuucAfaGfccugcsusg | 621 | CAGCAGGCTTGAAGCAGGGTAAG | 756 |
| AD-1657245 | gscsagggUfaAfGfCfaggcuggccuL96 | 487 | asGfsgccAfgCfCfugcuUfaCfccugesusu | 622 | AAGCAGGGTAAGCAGGCTGGCCC | 757 |
| AD-1657261 | gsgscccaGfgGfUfAfagggcuguuuL96 | 488 | asAfsacaGfcCfCfuuacCfcUfgggccsasg | 623 | CTGGCCCAGGGTAAGGGCTGTTG | 758 |
| AD-1657274 | gsgscuguUfgAfGfGfuaccuuaaguL96 | 489 | asCfsuuaAfgGfUfaccuCfaAfcagccscsu | 624 | AGGGCTGTTGAGGTACCTTAAGG | 759 |
| AD-1657286 | ascscuuaAfgGfGfAfaggucaagauL96 | 490 | asUfscuuGfaCfCfuuccCfuUfaaggusasc | 625 | GTACCTTAAGGGAAGGTCAAGAG | 760 |
| AD-1657299 | gsuscaagAfgGfGfAfgaugggcaauL96 | 491 | asUfsugcCfcAfUfcuccCfuCfuugacscsu | 626 | AGGTCAAGAGGGAGATGGGCAAG | 761 |
| AD-1657322 | gscsugagGfgAfGfGfaugcuuagguL96 | 492 | asCfscuaAfgCfAfuccuCfcCfucagesgsc | 627 | GCGCTGAGGGAGGATGCTTAGGG | 762 |
| AD-1657324 | asgsaaacAfgGfAfGfucaggaaaauL96 | 493 | asUfsuuuCfcUfGfacucCfuGfuuucusgsg | 628 | CCAGAAACAGGAGTCAGGAAAAT | 763 |
| AD-1657335 | uscsaggaAfaAfUfGfaggcacuaauL96 | 494 | asUfsuagUfgCfCfucauUfuUfccugascsu | 629 | AGTCAGGAAAATGAGGCACTAAG | 764 |
| AD-1657347 | gsgscacuAfaGfCfCfuaagaaguuuL96 | 495 | asAfsacuUfcUfUfaggcUfuAfgugccsusc | 630 | GAGGCACTAAGCCTAAGAAGTTC | 765 |
| AD-1657359 | asasgaagUfuCfCfCfugguuuuucuL96 | 496 | asGfsaaaAfaCfCfagggAfaCfuucuusasg | 631 | CTAAGAAGTTCCCTGGTTTTTCC | 766 |
| AD-1657374 | csascuggGfaGfAfCfaagcauuuauL96 | 497 | asUfsaaaUfgCfUfugucUfcCfcagugsgsg | 632 | CCCACTGGGAGACAAGCATTTAT | 767 |
| AD-1657385 | asasgcauUfuAfUfAfcuuucuuucuL96 | 498 | asGfsaaaGfaAfAfguauAfaAfugcuusgsu | 633 | ACAAGCATTTATACTTTCTTTCT | 768 |
| AD-1657395 | ususuugaGfaUfCfGfagucucgcuuL96 | 499 | asAfsgcgAfgAfCfucgaUfcUfcaaaasasa | 634 | TTTTTTGAGATCGAGTCTCGCTC | 769 |
| AD-1657410 | uscsucgcUfcUfGfUfcaccaggcuuL96 | 500 | asAfsgccUfgGfUfgacaGfaGfcgagascsu | 635 | AGTCTCGCTCTGTCACCAGGCTG | 770 |
| AD-1657431 | asgsugcaGfuGfAfCfacgaucuuguL96 | 501 | asCfsaagAfuCfGfugucAfcUfgcacuscsc | 636 | GGAGTGCAGTGACACGATCTTGG | 771 |
| AD-1657446 | gscsucacUfgCfAfAfccuccgucuuL96 | 502 | asAfsgacGfgAfGfguugCfaGfugagcscsa | 637 | TGGCTCACTGCAACCTCCGTCTC | 772 |
| AD-1657457 | cscsuccgUfcUfCfCfuggguucaauL96 | 503 | asUfsugaAfcCfCfaggaGfaCfggaggsusu | 638 | AACCTCCGTCTCCTGGGTTCAAG | 773 |
| AD-1657463 | ususcugcCfuCfAfGfccucccgaguL96 | 504 | asCfsucgGfgAfGfgcugAfgGfcagaasgsa | 639 | TCTTCTGCCTCAGCCTCCCGAGC | 774 |
| AD-1657475 | usgsgguuCfaAfGfUfgauucuucuuL96 | 505 | asAfsgaaGfaAfUfcacuUfgAfacccasgsg | 640 | CCTGGGTTCAAGTGATTCTTCTG | 775 |
| AD-1657503 | gsasgcagCfuGfGfGfauuacaggcuL96 | 506 | asGfsccuGfuAfAfucccAfgCfugcucsgsg | 641 | CCGAGCAGCTGGGATTACAGGCG | 776 |
| AD-1657520 | csgscccaCfuAfAfUfuuuuguauuuL96 | 507 | asAfsauaCfaAfAfaauuAfgUfgggcgscsc | 642 | GGCGCCCACTAATTTTTGTATTC | 777 |
| AD-1657529 | usgsuauuCfuUfAfGfuagaaacgauL96 | 508 | asUfscguUfuCfUfacuaAfgAfauacasasa | 643 | TTTGTATTCTTAGTAGAAACGAG | 778 |
| AD-1657540 | usasgaaaCfgAfGfGfuuucaacauuL96 | 509 | asAfsuguUfgAfAfaccuCfgUfuucuascsu | 644 | AGTAGAAACGAGGTTTCAACATG | 779 |
| AD-1657552 | ususcaacAfuGfUfUfggccaggauuL96 | 510 | asAfsuccUfgGfCfcaacAfuGfuugaasasc | 645 | GTTTCAACATGTTGGCCAGGATG | 780 |
| AD-1657564 | cscsaggaUfgGfUfCfucaaucucuuL96 | 511 | asAfsgagAfuUfGfagacCfaUfccuggscsc | 646 | GGCCAGGATGGTCTCAATCTCTT | 781 |
| AD-1657575 | uscsaaucUfcUfUfGfaccucuugauL96 | 512 | asUfscaaGfaGfGfucaaGfaGfauugasgsa | 647 | TCTCAATCTCTTGACCTCTTGAT | 782 |
| AD-1657586 | ascscucuUfgAfUfCfcacccgacuuL96 | 513 | asAfsgucGfgGfUfggauCfaAfgagguscsa | 648 | TGACCTCTTGATCCACCCGACTT | 783 |
| AD-1657600 | cscsgacuUfgGfCfCfucccgaaguuL96 | 514 | asAfscuuCfgGfGfaggcCfaAfgucggsgsu | 649 | ACCCGACTTGGCCTCCCGAAGTG | 784 |
| AD-1657615 | gsasagugAfuGfAfGfauuauaggcuL96 | 515 | asGfsccuAfuAfAfucucAfuCfacuucsgsg | 650 | CCGAAGTGATGAGATTATAGGCG | 785 |
| AD-1657641 | cscsuggcUfuAfUfAfcuuucuuaauL96 | 516 | asUfsuaaGfaAfAfguauAfaGfccaggscsg | 651 | CGCCTGGCTTATACTTTCTTAAT | 786 |
| AD-1657653 | gsasgaaaGfaAfAfAfucaacaaauuL96 | 517 | asAfsuuuGfuUfGfauuuUfcUfuucucscsu | 652 | AGGAGAAAGAAAATCAACAAATG | 787 |
| AD-1657674 | usgsagucAfuAfAfAfgaaggguuauL96 | 518 | asUfsaacCfcUfUfcuuuAfuGfacucascsa | 653 | TGTGAGTCATAAAGAAGGGTTAG | 788 |
| AD-1657687 | asgsgguuAfgGfGfUfgaugguccauL96 | 519 | asUfsggaCfcAfUfcaccCfuAfacccususc | 654 | GAAGGGTTAGGGTGATGGTCCAG | 789 |
| AD-1657704 | cscsagagCfaAfCfAfguucuucaauL96 | 520 | asUfsugaAfgAfAfcuguUfgCfucuggsasc | 655 | GTCCAGAGCAACAGTTCTTCAAG | 790 |
| AD-1657716 | ususcuucAfaGfUfGfuacucuguauL96 | 521 | asUfsacaGfaGfUfacacUfuGfaagaascsu | 656 | AGTTCTTCAAGTGTACTCTGTAG | 791 |
| AD-1657727 | usascucuGfuAfGfGfcuucugggauL96 | 522 | asUfscccAfgAfAfgccuAfcAfgaguascsa | 657 | TGTACTCTGTAGGCTTCTGGGAG | 792 |
| AD-1657741 | csusgggaGfgUfCfCfcuuuucagguL96 | 523 | asCfscugAfaAfAfgggaCfcUfcccagsasa | 658 | TTCTGGGAGGTCCCTTTTCAGGG | 793 |
| AD-1657744 | gsusccacAfaAfGfUfcaaagcuauuL96 | 524 | asAfsuagCfuUfUfgacuUfuGfuggacsasc | 659 | GTGTCCACAAAGTCAAAGCTATT | 794 |
| AD-1657760 | csusaacaUfgUfUfAfuuugccuuuuL96 | 525 | asAfsaagGfcAfAfauaaCfaUfguuagsusa | 660 | TACTAACATGTTATTTGCCTTTT | 795 |
| AD-1657776 | csusuuugAfaUfUfCfucauuaucuuL96 | 526 | asAfsgauAfaUfGfagaaUfuCfaaaagsgsc | 661 | GCCTTTTGAATTCTCATTATCTT | 796 |
| AD-1657793 | gsusauugUfgGfAfGfuuuuccagauL96 | 527 | asUfscugGfaAfAfacucCfaCfaauacsasa | 662 | TTGTATTGTGGAGTTTTCCAGAG | 797 |
| AD-1657811 | gsasggccGfuGfUfGfacaugugauuL96 | 528 | asAfsucaCfaUfGfucacAfcGfgccucsusg | 663 | CAGAGGCCGTGTGACATGTGATT | 798 |
| AD-1657822 | ascsauguGfaUfUfAfcaucaucuuuL96 | 529 | asAfsagaUfgAfUfguaaUfcAfcauguscsa | 664 | TGACATGTGATTACATCATCTTT | 799 |
| AD-1657834 | asuscaucUfuUfCfUfgacaucauuuL96 | 530 | asAfsaugAfuGfUfcagaAfaGfaugausgsu | 665 | ACATCATCTTTCTGACATCATTG | 800 |
| AD-1657845 | gsascaucAfuUfGfUfuaauggaauuL96 | 531 | asAfsuucCfaUfUfaacaAfuGfaugucsasg | 666 | CTGACATCATTGTTAATGGAATG | 801 |

TABLE 4
Unmodified Sense and Antisense Strand Sequences of ACVR1C dsRNA Agents
Comprising anUnsaturated C22 Hydrocarbon Chain Conjugated to Position 6
on the Sense Strand, Counting from the 5′-end of the Sense Strand

| | | SEQ | | | SEQ | |
|---|---|---|---|---|---|---|
| | | ID | Range in | Antisense | ID | Range in |
| Duplex Name | Sense Sequence 5′ to 3′ | NO: | NM_145259.3 | Sequence 5′ to 3′ | NO: | NM_145259.3 |
| AD-1735861 | UUUUUUCAAACUCCAUUCCUA | 802 | 3085-3105 | UAGGAAUGGAGUUUGAAAAAAGA | 1071 | 3083-3105 |
| AD-1735862 | ACUUUCAUCUGUCUUCAGAUA | 803 | 8677-8697 | UAUCUGAAGACAGAUGAAAGUUA | 1072 | 8675-8697 |
| AD-1735863 | CCUCAUAACUUCUCCAGUAAA | 804 | 6745-6765 | UUUACUGGAGAAGUUAUGAGGUG | 1073 | 6743-6765 |
| AD-1735864 | CUCACUAUCUUUCAACAUUAA | 805 | 5871-5891 | UUAAUGUUGAAAGAUAGUGAGGG | 1074 | 5869-5891 |
| AD-1735865 | CUCGUUAAUGCUCUCAUCCAA | 806 | 3562-3582 | UUGGAUGAGAGCAUUAACGAGUA | 1075 | 3560-3582 |
| AD-1735866 | GUUCUUCAUAAUCCACUACUA | 807 | 2292-2312 | UAGUAGUGGAUUAUGAAGAACAG | 1076 | 2290-2312 |
| AD-1735867 | GUUGACUUCAUCCAAUCUCUA | 808 | 1876-1896 | UAGAGAUUGGAUGAAGUCAACUC | 1077 | 1874-1896 |
| AD-1735868 | CUACACAAUGAACUUCUUAAA | 809 | 6692-6712 | UUUAAGAAGUUCAUUGUGUAGAA | 1078 | 6690-6712 |
| AD-1735869 | CACAUCUAGAAUUCUUAAUUA | 810 | 4835-4855 | UAAUUAAGAAUUCUAGAUGUGAC | 1079 | 4833-4855 |
| AD-1735870 | AAAUCUCUCAUAGCUUUCUUA | 811 | 1725-1745 | UAAGAAAGCUAUGAGAGAUUUCU | 1080 | 1723-1745 |
| AD-1735871 | CCUACUAUUGUAGAAUUACUA | 812 | 5093-5113 | UAGUAAUUCUACAAUAGUAGGUG | 1081 | 5091-5113 |
| AD-1735872 | AUCUUCUUUUAGUGCAUUAAA | 813 | 6589-6609 | UUUAAUGCACUAAAAGAAGAUGA | 1082 | 6587-6609 |
| AD-1735873 | CUCAUGUACUCUUCUGAUUCA | 814 | 7977-7997 | UGAAUCAGAAGAGUACAUGAGCU | 1083 | 7975-7997 |
| AD-1735874 | UUUCUCUUUGUACCUUGGAAA | 815 | 6449-6469 | UUUCCAAGGUACAAAGAGAAAGA | 1084 | 6447-6469 |
| AD-1735875 | UGGCUUAUACAUCUUCAGAAA | 816 | 7515-7535 | UUUCUGAAGAUGUAUAAGCCAGA | 1085 | 7513-7535 |
| AD-1735876 | CAAAACAAAACUACUCCUAUA | 817 | 5120-5140 | UAUAGGAGUAGUUUUGUUUUGUU | 1086 | 5118-5140 |
| AD-1735877 | ACUAGCAGAACUCUUAUGAAA | 818 | 6055-6075 | UUUCAUAAGAGUUCUGCUAGUAA | 1087 | 6053-6075 |
| AD-1735878 | CCAAGUCACUCUAUAAUUCCA | 819 | 6635-6655 | UGGAAUUAUAGAGUGACUUGGGA | 1088 | 6633-6655 |
| AD-1735879 | UCCAUUUUUCUUGUCAUUAUA | 820 | 4114-4134 | UAUAAUGACAAGAAAAAUGGAGA | 1089 | 4112-4134 |
| AD-1735880 | CAACACCUCAACUCAUCUUUA | 821 | 1923-1943 | UAAAGAUGAGUUGAGGUGUUGCU | 1090 | 1921-1943 |
| AD-1735881 | ACUCUGAAAGAUCUGAUUUAA | 822 | 716-736 | UUAAAUCAGAUCUUUCAGAGUUU | 1091 | 714-736 |
| AD-1735882 | CUAGUUCUUUUCCGCAAUGAA | 823 | 7946-7966 | UUCAUUGCGGAAAAGAACUAGAA | 1092 | 7944-7966 |
| AD-1735883 | CUUUCACUCUGAAGAAAUCCA | 824 | 4189-4209 | UGGAUUUCUUCAGAGUGAAAGUU | 1093 | 4187-4209 |
| AD-1735884 | UCUAACUUUUCUCUCUUUCUA | 825 | 6525-6545 | UAGAAAGAGAGAAAAGUUAGAUA | 1094 | 6523-6545 |
| AD-1735885 | UCUCAACUUUGUGUCAAAGAA | 826 | 1667-1687 | UUCUUUGACACAAAGUUGAGAUA | 1095 | 1665-1687 |
| AD-1735886 | CUUCAAUAAUCAUUCCUUUAA | 827 | 5445-5465 | UUAAAGGAAUGAUUAUUGAAGAC | 1096 | 5443-5465 |
| AD-1735887 | UAACUGCUCUUCGUAUUAAGA | 828 | 1638-1658 | UCUUAAUACGAAGAGCAGUUAGG | 1097 | 1636-1658 |
| AD-1735888 | UCUUCUGUCAUAGUUCCAACA | 829 | 438-458 | UGUUGGAACUAUGACAGAAGACU | 1098 | 436-458 |
| AD-1735889 | AUCUCAGAACCAUAUCUGUUA | 830 | 2584-2604 | UAACAGAUAUGGUUCUGAGAUUU | 1099 | 2582-2604 |
| AD-1735890 | UGAAUCUAUCUUCAUUUUACA | 831 | 3483-3503 | UGUAAAAUGAAGAUAGAUUCAGU | 1100 | 3481-3503 |
| AD-1735891 | ACCACUAAACUUGUUCCUUUA | 832 | 2326-2346 | UAAAGGAACAAGUUUAGUGGUUC | 1101 | 2324-2346 |
| AD-1735892 | AGUGUUCUUUACUCCUUACAA | 833 | 4578-4598 | UUGUAAGGAGUAAAGAACACUGA | 1102 | 4576-4598 |
| AD-1735893 | CUUUACUCUUAACAGGAUUAA | 834 | 4367-4387 | UUAAUCCUGUUAAGAGUAAAGGA | 1103 | 4365-4387 |
| AD-1735894 | UAUACCUAAGAACAUAUUACA | 835 | 8153-8173 | UGUAAUAUGUUCUUAGGUAUAAG | 1104 | 8151-8173 |
| AD-1735895 | CUUCUACUGAGAUGAUCCAAA | 836 | 3114-3134 | UUUGGAUCAUCUCAGUAGAAGAA | 1105 | 3112-3134 |
| AD-1735896 | CUGUAUUUUCUCAUAGAGUAA | 837 | 3420-3440 | UUACUCUAUGAGAAAAUACAGAA | 1106 | 3418-3440 |
| AD-1735897 | ACUUUUCUCUUUCAGUUGUAA | 838 | 7116-7136 | UUACAACUGAAAGAGAAAAGUAU | 1107 | 7114-7136 |
| AD-1735898 | AUUAAUUUCUAGUCUGUGAAA | 839 | 8493-8513 | UUUCACAGACUAGAAAUUAAUGA | 1108 | 8491-8513 |
| AD-1735899 | CUCUGUAUAAGAGGUUUCACA | 840 | 6726-6746 | UGUGAAACCUCUUAUACAGAGAA | 1109 | 6724-6746 |
| AD-1735900 | GACUUUCAAAGUACUAAUACA | 841 | 8098-8118 | UGUAUUAGUACUUUGAAAGUCAA | 1110 | 8096-8118 |
| AD-1735901 | UUUGCCAUUAUACAAAGUUUA | 842 | 5262-5282 | UAAACUUUGUAUAAUGGCAAACA | 1111 | 5260-5282 |
| AD-1735902 | GAACACUAUCGACAUACCUCA | 843 | 1276-1296 | UGAGGUAUGUCGAUAGUGUUCAG | 1112 | 1274-1296 |
| AD-1735903 | UAGAAUGACAUUUACUAAUAA | 844 | 7818-7838 | UUAUUAGUAAAUGUCAUUCUACG | 1113 | 7816-7838 |
| AD-1735904 | AAGCUACUUAGAAAUGUUUAA | 845 | 5491-5511 | UUAAACAUUUCUAAGUAGCUUGU | 1114 | 5489-5511 |
| AD-1735905 | CCUUAGUACUCUCAGAGAUUA | 846 | 5968-5988 | UAAUCUCUGAGAGUACUAAGGGA | 1115 | 5966-5988 |
| AD-1735906 | UGAAUGCACACUAACGUAAUA | 847 | 6163-6183 | UAUUACGUUAGUGUGCAUUCAAG | 1116 | 6161-6183 |
| AD-1735907 | AAGCCUAAUGAUGAUAAUUAA | 848 | 1695-1715 | UUAAUUAUCAUCAUUAGGCUUUG | 1117 | 1693-1715 |
| AD-1735908 | AAACUUUUUACUAUCCCAUAA | 849 | 3341-3361 | UUAUGGGAUAGUAAAAAGUUUCC | 1118 | 3339-3361 |
| AD-1735909 | CUAGCUGAUACUCUUAAGUAA | 850 | 8268-8288 | UUACUUAAGAGUAUCAGCUAGCU | 1119 | 8266-8288 |
| AD-1735910 | AGCAUGAAAAGAUAACUCUAA | 851 | 1837-1857 | UUAGAGUUAUCUUUUCAUGCUGC | 1120 | 1835-1857 |
| AD-1735911 | UAAGCCAUUAUGCUAUUAGUA | 852 | 7017-7037 | UACUAAUAGCAUAAUGGCUUAGU | 1121 | 7015-7037 |
| AD-1735912 | UCUAGCUUUUAGCUAACAUAA | 853 | 6850-6870 | UUAUGUUAGCUAAAAGCUAGAAU | 1122 | 6848-6870 |
| AD-1735913 | GUGUGAUUCUUCAAACUUCAA | 854 | 316-336 | UUGAAGUUUGAAGAAUCACACAA | 1123 | 314-336 |
| AD-1735914 | UCUCCAGACCUAACAGUUUUA | 855 | 7247-7267 | UAAAACUGUUAGGUCUGGAGAGA | 1124 | 7245-7267 |
| AD-1735915 | CUUUUUUUCAUCUAGCCUUGA | 856 | 5808-5828 | UCAAGGCUAGAUGAAAAAAAGAA | 1125 | 5806-5828 |
| AD-1735916 | CAAAAUAAUUCUUGACAUCUA | 857 | 7552-7572 | UAGAUGUCAAGAAUUAUUUUGAC | 1126 | 7550-7572 |
| AD-1735917 | GCAUCACUUUCUGAAAAUAAA | 858 | 4690-4710 | UUUAUUUUCAGAAAGUGAUGCUG | 1127 | 4688-4710 |
| AD-1735918 | UCUUUUGUGAAACAUACUAUA | 859 | 5563-5583 | UAUAGUAUGUUUCACAAAAGAUG | 1128 | 5561-5583 |
| AD-1735919 | CUAUUUCUUCCAUAGGCUAAA | 860 | 7841-7861 | UUUAGCCUAUGGAAGAAAUAGAA | 1129 | 7839-7861 |
| AD-1735920 | CUUCAAUGAAGUGUUAACAAA | 861 | 2839-2859 | UUUGUUAACACUUCAUUGAAGAG | 1130 | 2837-2859 |
| AD-1735921 | UUUGCCUUCAUUCUACUUUCA | 862 | 2952-2972 | UGAAAGUAGAAUGAAGGCAAAAC | 1131 | 2950-2972 |
| AD-1735922 | AGGUUUUUAAAUGUCCUAAAA | 863 | 8035-8055 | UUUUAGGACAUUUAAAAACCUGA | 1132 | 8033-8055 |
| AD-1735923 | GAAUAUCUUUGAGUCCUUCAA | 864 | 1360-1380 | UUGAAGGACUCAAAGAUAUUCAC | 1133 | 1358-1380 |
| AD-1735924 | GAACACUUCCAAAGAUUAAUA | 865 | 2666-2686 | UAUUAAUCUUUGGAAGUGUUCAG | 1134 | 2664-2686 |
| AD-1735925 | CUAAAUUUUGUCUAACAAAUA | 866 | 7791-7811 | UAUUUGUUAGACAAAAUUUAGGC | 1135 | 7789-7811 |
| AD-1735926 | GUAAACAACUUAAAAUUGCUA | 867 | 5284-5304 | UAGCAAUUUUAAGUUGUUUACUC | 1136 | 5282-5304 |
| AD-1735927 | AAAAUAAUUUACUCACUCAGA | 868 | 6233-6253 | UCUGAGUGAGUAAAUUAUUUUAG | 1137 | 6231-6253 |
| AD-1735928 | GACAUCUAUUCUGUUGGUCUA | 869 | 1388-1408 | UAGACCAACAGAAUAGAUGUCAG | 1138 | 1386-1408 |
| AD-1735929 | UAACUGAGUAGUCUUAUAUUA | 870 | 4267-4287 | UAAUAUAAGACUACUCAGUUAGG | 1139 | 4265-4287 |
| AD-1735930 | UCUGGAAUUUAGCCUUCUAAA | 871 | 5936-5956 | UUUAGAAGGCUAAAUUCCAGACG | 1140 | 5934-5956 |
| AD-1735931 | ACAUGCCUUGAACUCUUGAAA | 872 | 8207-8227 | UUUCAAGAGUUCAAGGCAUGUUU | 1141 | 8205-8227 |
| AD-1735932 | AUCAAGCACUUCUUGCACUUA | 873 | 8782-8802 | UAAGUGCAAGAAGUGCUUGAUUU | 1142 | 8780-8802 |
| AD-1735933 | CUUGCUUUUCAUCUUUAUACA | 874 | 4915-4935 | UGUAUAAAGAUGAAAAGCAAGUG | 1143 | 4913-4935 |
| AD-1735934 | UUUUCUCCCUAUGUAAUAUCA | 875 | 2493-2513 | UGAUAUUACAUAGGGAGAAAAGC | 1144 | 2491-2513 |
| AD-1735935 | UGCUUUCUUCUAUGUCUAAAA | 876 | 4802-4822 | UUUUAGACAUAGAAGAAAGCAUA | 1145 | 4800-4822 |
| AD-1735936 | ACCUGCUUAGCAAAUCGUCUA | 877 | 5224-5244 | UAGACGAUUUGCUAAGCAGGUUC | 1146 | 5222-5244 |
| AD-1735937 | AUAUUCCUCUACCCUUUCACA | 878 | 2895-2915 | UGUGAAAGGGUAGAGGAAUAUCU | 1147 | 2893-2915 |
| AD-1735938 | UCCCACUUUUCUUUGGGUUUA | 879 | 6208-6228 | UAAACCCAAAGAAAAGUGGGAUU | 1148 | 6206-6228 |
| AD-1735939 | AGGCUUCUUGUAUAUACUUAA | 880 | 4442-4462 | UUAAGUAUAUACAAGAAGCCUGC | 1149 | 4440-4462 |
| AD-1735940 | AAACUUGAUCUGUGUUUCAGA | 881 | 8017-8037 | UCUGAAACACAGAUCAAGUUUGA | 1150 | 8015-8037 |
| AD-1735941 | UUUGCUUUAAUGCUAAAUUCA | 882 | 6885-6905 | UGAAUUUAGCAUUAAAGCAAAAG | 1151 | 6883-6905 |
| AD-1735942 | AGACUAGCUGUUAUUUGUAUA | 883 | 6830-6850 | UAUACAAAUAACAGCUAGUCUUU | 1152 | 6828-6850 |
| AD-1735943 | UUCCUACUUUCCCUUGAAUAA | 884 | 2469-2489 | UUAUUCAAGGGAAAGUAGGAAGC | 1153 | 2467-2489 |
| AD-1735944 | CAGAACUGAAUGCUCAAGUCA | 885 | 420-440 | UGACUUGAGCAUUCAGUUCUGGA | 1154 | 418-440 |
| AD-1735945 | UCUUGUUACUAAUUUCUCAUA | 886 | 3945-3965 | UAUGAGAAAUUAGUAACAAGAAC | 1155 | 3943-3965 |
| AD-1735946 | CUCAGUGAUAGCCUUUAUACA | 887 | 8586-8606 | UGUAUAAAGGCUAUCACUGAGUU | 1156 | 8584-8606 |
| AD-1735947 | UUUUAUGCUCCUAAAACAUCA | 888 | 3065-3085 | UGAUGUUUUAGGAGCAUAAAAGA | 1157 | 3063-3085 |
| AD-1735948 | UUCUCCUUAGGUUAUGUUCAA | 889 | 4551-4571 | UUGAACAUAACCUAAGGAGAAUU | 1158 | 4549-4571 |
| AD-1735949 | AUUGCUCAUCGAGACAUAAAA | 890 | 1175-1195 | UUUUAUGUCUCGAUGAGCAAUAG | 1159 | 1173-1195 |
| AD-1735950 | CCUUUGUACAUACACACUUAA | 891 | 2746-2766 | UUAAGUGUGUAUGUACAAAGGAA | 1160 | 2744-2766 |
| AD-1735951 | UUGAACCAAGAGCACAUGAAA | 892 | 2025-2045 | UUUCAUGUGCUCUUGGUUCAAGU | 1161 | 2023-2045 |
| AD-1735952 | UCAGGGAUUUUAAAGUCUAAA | 893 | 3668-3688 | UUUAGACUUUAAAAUCCCUGAGG | 1162 | 3666-3688 |
| AD-1735953 | UACUUGCUUAUAAAGUGAUAA | 894 | 3153-3173 | UUAUCACUUUAUAAGCAAGUACC | 1163 | 3151-3173 |
| AD-1735954 | AUGCAUCUAAACCUACCUUGA | 895 | 6145-6165 | UCAAGGUAGGUUUAGAUGCAUGC | 1164 | 6143-6165 |
| AD-1735955 | UUGGGAAAACACUAUUAUGAA | 896 | 4629-4649 | UUCAUAAUAGUGUUUUCCCAAGU | 1165 | 4627-4649 |
| AD-1735956 | AAACUCCAAAGAGAAAUGAAA | 897 | 8456-8476 | UUUCAUUUCUCUUUGGAGUUUGA | 1166 | 8454-8476 |
| AD-1735957 | AUGGGCAUUUUUCAAAACAUA | 898 | 5758-5778 | UAUGUUUUGAAAAAUGCCCAUAU | 1167 | 5756-5778 |
| AD-1735958 | AUAAUGGAACUUGGACUCAAA | 899 | 996-1016 | UUUGAGUCCAAGUUCCAUUAUCU | 1168 | 994-1016 |
| AD-1735959 | UGUGUAGAUGACGAAACCAAA | 900 | 6008-6028 | UUUGGUUUCGUCAUCUACACAAA | 1169 | 6006-6028 |
| AD-1735960 | UUCUACCUCAAAGAUAAGACA | 901 | 1790-1810 | UGUCUUAUCUUUGAGGUAGAACA | 1170 | 1788-1810 |
| AD-1735961 | AGAAGGCAUAUUGAGUUAUUA | 902 | 2536-2556 | UAAUAACUCAAUAUGCCUUCUAA | 1171 | 2534-2556 |
| AD-1735962 | AUGGAGUUUUUCUGCUAUUUA | 903 | 5629-5649 | UAAAUAGCAGAAAAACUCCAUGU | 1172 | 5627-5649 |
| AD-1735963 | UCAGUUUCUCAUUAGUUUGUA | 904 | 6558-6578 | UACAAACUAAUGAGAAACUGAGG | 1173 | 6556-6578 |
| AD-1735964 | CUGCAUAUAUAAGAACGAAAA | 905 | 3976-3996 | UUUUCGUUCUUAUAUAUGCAGAA | 1174 | 3974-3996 |
| AD-1735965 | UGAGCCAAAAUAUUAAAUUCA | 906 | 7927-7947 | UGAAUUUAAUAUUUUGGCUCAGA | 1175 | 7925-7947 |
| AD-1735966 | AGAAUGAUAUUUCCUGUUUUA | 907 | 4035-4055 | UAAAACAGGAAAUAUCAUUCUUG | 1176 | 4033-4055 |
| AD-1735967 | GUGCUCUAUAUAAAUCUUCCA | 908 | 7385-7405 | UGGAAGAUUUAUAUAGAGCACAA | 1177 | 7383-7405 |
| AD-1735968 | UUUACAGAGAAGCAUCUUAUA | 909 | 7468-7488 | UAUAAGAUGCUUCUCUGUAAAAU | 1178 | 7466-7488 |
| AD-1735969 | GUCUUGAGUUCUGCAUGACAA | 910 | 3761-3781 | UUGUCAUGCAGAACUCAAGACAU | 1179 | 3759-3781 |
| AD-1735970 | CUUCAAAUCUAUAAUUUUACA | 911 | 3017-3037 | UGUAAAAUUAUAGAUUUGAAGGG | 1180 | 3015-3037 |
| AD-1735971 | GAAGCAUAUAGAACUCUAUUA | 912 | 8810-8830 | UAAUAGAGUUCUAUAUGCUUCAC | 1181 | 8808-8830 |
| AD-1735972 | GAGCUGCUUCUAAAUAACAAA | 913 | 5678-5698 | UUUGUUAUUUAGAAGCAGCUCUC | 1182 | 5676-5698 |
| AD-1735973 | UGACUGUAUUUCCUGAUCAUA | 914 | 7197-7217 | UAUGAUCAGGAAAUACAGUCACA | 1183 | 7195-7217 |
| AD-1735974 | GUUCUGAAGGAUAUACUUCUA | 915 | 3402-3422 | UAGAAGUAUAUCCUUCAGAACGC | 1184 | 3400-3422 |
| AD-1735975 | GUGAAGCAUGAUUCAAUACUA | 916 | 1256-1276 | UAGUAUUGAAUCAUGCUUCACAG | 1185 | 1254-1276 |
| AD-1735976 | AAUGCUGCUUCACAGAUUUUA | 917 | 474-494 | UAAAAUCUGUGAAGCAGCAUUCG | 1186 | 472-494 |
| AD-1735977 | UGUGGGUUAUGUUAAUCUGAA | 918 | 7283-7303 | UUCAGAUUAACAUAACCCACAGU | 1187 | 7281-7303 |
| AD-1735978 | GAGUACAGAAGAAUGCUCAUA | 919 | 2926-2946 | UAUGAGCAUUCUUCUGUACUCAC | 1188 | 2924-2946 |
| AD-1735979 | CUGUAUUUCAUCCUAGAUUUA | 920 | 7162-7182 | UAAAUCUAGGAUGAAAUACAGCU | 1189 | 7160-7182 |
| AD-1735980 | AAUCUGGUAAAUGCUGGAAAA | 921 | 695-715 | UUUUCCAGCAUUUACCAGAUUGC | 1190 | 693-715 |
| AD-1735981 | UGCUCAGAUUACCUGAUCGUA | 922 | 3293-3313 | UACGAUCAGGUAAUCUGAGCAAU | 1191 | 3291-3313 |
| AD-1735982 | GACAGCUAUGGAGUUUGCGUA | 923 | 3531-3551 | UACGCAAACUCCAUAGCUGUCAG | 1192 | 3529-3551 |
| AD-1735983 | UAGAACAUGCUCACUUACAAA | 924 | 4391-4411 | UUUGUAAGUGAGCAUGUUCUAUA | 1193 | 4389-4411 |
| AD-1735984 | AUUAUGCUUCAUUUCACUAUA | 925 | 3450-3470 | UAUAGUGAAAUGAAGCAUAAUCA | 1194 | 3448-3470 |
| AD-1735985 | CAACAGCAAGUCAUAAAAGUA | 926 | 8624-8644 | UACUUUUAUGACUUGCUGUUGGU | 1195 | 8622-8644 |
| AD-1735986 | UUGUCUGAAAAUGUCUUUAAA | 927 | 2702-2722 | UUUAAAGACAUUUUCAGACAAUG | 1196 | 2700-2722 |
| AD-1735987 | UGUUGGUUCAAAGGACAAUUA | 928 | 774-794 | UAAUUGUCCUUUGAACCAACAGA | 1197 | 772-794 |
| AD-1735988 | UCUAAACUUUAAUAUAUCGAA | 929 | 2190-2210 | UUCGAUAUAUUAAAGUUUAGAGC | 1198 | 2188-2210 |
| AD-1735989 | AAGUGAAACAUGACUGUCGUA | 930 | 4875-4895 | UACGACAGUCAUGUUUCACUUAA | 1199 | 4873-4895 |
| AD-1735990 | AUCGUCCAUACAGUUUUCAUA | 931 | 7719-7739 | UAUGAAAACUGUAUGGACGAUAU | 1200 | 7717-7739 |
| AD-1735991 | CUUCAGGAAAUAGUAGGAAAA | 932 | 809-829 | UUUUCCUACUAUUUCCUGAAGCA | 1201 | 807-829 |
| AD-1735992 | CACCCUUUCUGUAAGGACUGA | 933 | 6426-6446 | UCAGUCCUUACAGAAAGGGUGGG | 1202 | 6424-6446 |
| AD-1735993 | AAAGAGAUACCUGACAUCCUA | 934 | 2354-2374 | UAGGAUGUCAGGUAUCUCUUUUG | 1203 | 2352-2374 |
| AD-1735994 | AUGAGCAUUAAUGUUUUCUGA | 935 | 2647-2667 | UCAGAAAACAUUAAUGCUCAUUA | 1204 | 2645-2667 |
| AD-1735995 | UGACAUUUUUUCUCAUAGAAA | 936 | 2603-2623 | UUUCUAUGAGAAAAAAUGUCAAC | 1205 | 2601-2623 |
| AD-1735996 | CAGAGAUUAAAUGACUACUAA | 937 | 6032-6052 | UUAGUAGUCAUUUAAUCUCUGAU | 1206 | 6030-6052 |
| AD-1735997 | GCUGUCAAAUGUUAUAUUGUA | 938 | 4463-4483 | UACAAUAUAACAUUUGACAGCAU | 1207 | 4461-4483 |
| AD-1735998 | AAUAUAAUACUACAGCAAAAA | 939 | 7041-7061 | UUUUUGCUGUAGUAUUAUAUUCC | 1208 | 7039-7061 |
| AD-1735999 | GGUGUCUAAAUAUAAUUUCAA | 940 | 4153-4173 | UUGAAAUUAUAUUUAGACACCCU | 1209 | 4151-4173 |
| AD-1736000 | CUCUUUAUACAAAAUGAGAGA | 941 | 7903-7923 | UCUCUCAUUUUGUAUAAAGAGAA | 1210 | 7901-7923 |
| AD-1736001 | GCAGUACCAAAUGAAUAAAAA | 942 | 5410-5430 | UUUUUAUUCAUUUGGUACUGCUA | 1211 | 5408-5430 |
| AD-1736002 | UUGUUGGUACACAAGGUAAAA | 943 | 1149-1169 | UUUUACCUUGUGUACCAACAAUC | 1212 | 1147-1169 |
| AD-1736003 | CUCUUAUUUAACUUAACCGUA | 944 | 5917-5937 | UACGGUUAAGUUAAAUAAGAGUU | 1213 | 5915-5937 |
| AD-1736004 | UGCUUGAUGAUACAAUGAAUA | 945 | 1338-1358 | UAUUCAUUGUAUCAUCAAGCAUU | 1214 | 1336-1358 |
| AD-1736005 | AACUGGGAAAGACAAUUUGAA | 946 | 6920-6940 | UUCAAAUUGUCUUUCCCAGUUAC | 1215 | 6918-6940 |
| AD-1736006 | CUCCUUAUAUGACUAUUUGAA | 947 | 1048-1068 | UUCAAAUAGUCAUAUAAGGAGCC | 1216 | 1046-1068 |
| AD-1736007 | AGUCAUGCUAACCAAUGGAAA | 948 | 367-387 | UUUCCAUUGGUUAGCAUGACUGA | 1217 | 365-387 |
| AD-1736008 | CCUGAAGUCACACAGCUAAUA | 949 | 2391-2411 | UAUUAGCUGUGUGACUUCAGGAC | 1218 | 2389-2411 |
| AD-1736009 | UUGCUGCUGACAACAAAGAUA | 950 | 978-998 | UAUCUUUGUUGUCAGCAGCAAUG | 1219 | 976-998 |
| AD-1736010 | UACUGACUUUGCCUGUAAAGA | 951 | 7757-7777 | UCUUUACAGGCAAAGUCAGUAAU | 1220 | 7755-7777 |
| AD-1736011 | AAUCCCUAUUUUAAAAUUCCA | 952 | 7070-7090 | UGGAAUUUUAAAAUAGGGAUUGU | 1221 | 7068-7090 |
| AD-1736012 | GUGCCUUAGGAGAUUACCCUA | 953 | 7330-7350 | UAGGGUAAUCUCCUAAGGCACUC | 1222 | 7328-7350 |
| AD-1736013 | GACAAUCUAAAUAUAUCAUCA | 954 | 3857-3877 | UGAUGAUAUAUUUAGAUUGUCUG | 1223 | 3855-3877 |
| AD-1736014 | UGUGAUAAUGAACACCGUAAA | 955 | 2438-2458 | UUUACGGUGUUCAUUAUCACAAG | 1224 | 2436-2458 |
| AD-1736015 | UCACCGUUGUAAAGACUUUUA | 956 | 8242-8262 | UAAAAGUCUUUACAACGGUGAUU | 1225 | 8240-8262 |
| AD-1736016 | UUAGAGAAGCUUCCUGACAUA | 957 | 6402-6422 | UAUGUCAGGAAGCUUCUCUAAUC | 1226 | 6400-6422 |
| AD-1736017 | AACAGUAAAGAAAUGCUACUA | 958 | 3252-3272 | UAGUAGCAUUUCUUUACUGUUAU | 1227 | 3250-3272 |
| AD-1736018 | UAUGUUCUGACUUGAGAGUUA | 959 | 4855-4875 | UAACUCUCAAGUCAGAACAUAAA | 1228 | 4853-4875 |
| AD-1736019 | UUAGAGCAUGCUAUCUUUAGA | 960 | 2109-2129 | UCUAAAGAUAGCAUGCUCUAAUU | 1229 | 2107-2129 |
| AD-1736020 | UAGUCUUUGAUUGAAAUAAGA | 961 | 5314-5334 | UCUUAUUUCAAUCAAAGACUAUG | 1230 | 5312-5334 |
| AD-1736021 | UUGGUAAACGAGAUUUAACAA | 962 | 3038-3058 | UUGUUAAAUCUCGUUUACCAAAG | 1231 | 3036-3058 |
| AD-1736022 | GUCUGAAAAUUGCUUUCAUUA | 963 | 3798-3818 | UAAUGAAAGCAAUUUUCAGACCU | 1232 | 3796-3818 |
| AD-1736023 | AUGAACUUGUUGCCUUGUAAA | 964 | 4741-4761 | UUUACAAGGCAACAAGUUCAUGC | 1233 | 4739-4761 |
| AD-1736024 | GGGCAAUAAACUGUAUCAAAA | 965 | 8379-8399 | UUUUGAUACAGUUUAUUGCCCAA | 1234 | 8377-8399 |
| AD-1736025 | ACAUGUUAGCAUAUAAUGUAA | 966 | 3175-3195 | UUACAUUAUAUGCUAACAUGUAC | 1235 | 3173-3195 |
| AD-1736026 | UGUGAGAGUAUAGAAUUUCUA | 967 | 6367-6387 | UAGAAAUUCUAUACUCUCACAGU | 1236 | 6365-6387 |
| AD-1736027 | AUCCUUCAUUUGGCACUAUAA | 968 | 6764-6784 | UUAUAGUGCCAAAUGAAGGAUUA | 1237 | 6762-6784 |
| AD-1736028 | ACAUUUAGAAAGUAGCUUUAA | 969 | 3824-3844 | UUAAAGCUACUUUCUAAAUGUGU | 1238 | 3822-3844 |
| AD-1736029 | AACUGUUCUAUAAAGCAAAGA | 970 | 3364-3384 | UCUUUGCUUUAUAGAACAGUUAA | 1239 | 3362-3384 |
| AD-1736030 | CCUCCAGAGAUGAAAGAUCUA | 971 | 894-914 | UAGAUCUUUCAUCUCUGGAGGAG | 1240 | 892-914 |
| AD-1736031 | UUGUUUAACUAUGACUCCUAA | 972 | 4249-4269 | UUAGGAGUCAUAGUUAAACAACA | 1241 | 4247-4269 |
| AD-1736032 | CUGUUUGACAAUGCUUUGUUA | 973 | 5585-5605 | UAACAAAGCAUUGUCAAACAGAA | 1242 | 5583-5605 |
| AD-1736033 | UGUCCUAAAAGAAAUUUUUCA | 974 | 7227-7247 | UGAAAAAUUUCUUUUAGGACAAC | 1243 | 7225-7247 |
| AD-1736034 | ACAGAUUGAACAAAGAACUUA | 975 | 7572-7592 | UAAGUUCUUUGUUCAAUCUGUAG | 1244 | 7570-7592 |
| AD-1736035 | GUUUAACCUGUCCAAACUUCA | 976 | 6673-6693 | UGAAGUUUGGACAGGUUAAACGA | 1245 | 6671-6693 |
| AD-1736036 | CAAAAUGUUUAACUUUACCAA | 977 | 8606-8626 | UUGGUAAAGUUAAACAUUUUGGU | 1246 | 8604-8626 |
| AD-1736037 | UCCCAUUGUGUAAUAUUUAUA | 978 | 4494-4514 | UAUAAAUAUUACACAAUGGGACU | 1247 | 4492-4514 |
| AD-1736038 | GUUGGGUAAAUAUGCUUAUUA | 979 | 4065-4085 | UAAUAAGCAUAUUUACCCAACAU | 1248 | 4063-4085 |
| AD-1736039 | CAGGGUUGUCUUUGAGUCUGA | 980 | 5187-5207 | UCAGACUCAAAGACAACCCUGAG | 1249 | 5185-5207 |
| AD-1736040 | GCUAAAAUCCAACUGCAAUUA | 981 | 7425-7445 | UAAUUGCAGUUGGAUUUUAGCCC | 1250 | 7423-7445 |
| AD-1736041 | CAGGACUGAAGUGUGUAUGUA | 982 | 291-311 | UACAUACACACUUCAGUCCUGGC | 1251 | 289-311 |
| AD-1736042 | GAGUACCAAUUGCCUUAUUAA | 983 | 1457-1477 | UUAAUAAGGCAAUUGGUACUCCU | 1252 | 1455-1477 |
| AD-1736043 | AAGUGCCUAUGUGAAGUGAUA | 984 | 3886-3906 | UAUCACUUCACAUAGGCACUUUU | 1253 | 3884-3906 |
| AD-1736044 | UAACAAUAAUAGAACUGCCAA | 985 | 7092-7112 | UUGGCAGUUCUAUUAUUGUUAGG | 1254 | 7090-7112 |
| AD-1736045 | GCUGUUCCAUACCAUUGCUUA | 986 | 6978-6998 | UAAGCAAUGGUAUGGAACAGCAG | 1255 | 6976-6998 |
| AD-1736046 | UAGACUGGAGAAGAUUAUUCA | 987 | 7997-8017 | UGAAUAAUCUUCUCCAGUCUAGA | 1256 | 7995-8017 |
| AD-1736047 | UUCCAACAGCAUCACCAAAUA | 988 | 516-536 | UAUUUGGUGAUGCUGUUGGAAGG | 1257 | 514-536 |
| AD-1736048 | UAAUUGGUACAGAUUCUGUUA | 989 | 4779-4799 | UAACAGAAUCUGUACCAAUUACA | 1258 | 4777-4799 |
| AD-1736049 | GAGAAACAUUAUUUGUUGUCA | 990 | 4710-4730 | UGACAACAAAUAAUGUUUCUCUU | 1259 | 4708-4730 |
| AD-1736050 | CGCUCUCAAUUGCUAGUGGUA | 991 | 1107-1127 | UACCACUAGCAAUUGAGAGCGCC | 1260 | 1105-1127 |
| AD-1736051 | CGGUAUCAGAAACAGCAAAUA | 992 | 2873-2893 | UAUUUGCUGUUUCUGAUACCGUC | 1261 | 2871-2893 |
| AD-1736052 | CCCAGACAUAAUUUUAUAUUA | 993 | 7449-7469 | UAAUAUAAAAUUAUGUCUGGGAG | 1262 | 7447-7469 |
| AD-1736053 | UAGUACAUUUUGAGGUAUUUA | 994 | 4215-4235 | UAAAUACCUCAAAAUGUACUAAC | 1263 | 4213-4235 |
| AD-1736054 | AAAUGCUAUUGAUAACAGUAA | 995 | 3211-3231 | UUACUGUUAUCAAUAGCAUUUAC | 1264 | 3209-3231 |
| AD-1736055 | UAGCUGAAUUUGGGACUAUGA | 996 | 6490-6510 | UCAUAGUCCCAAAUUCAGCUAUC | 1265 | 6488-6510 |
| AD-1736056 | AUUGUGGUUUUCAAAGAUAUA | 997 | 5535-5555 | UAUAUCUUUGAAAACCACAAUGG | 1266 | 5533-5555 |
| AD-1736057 | CCUGGUUGCAGUAUCUGAAAA | 998 | 4985-5005 | UUUUCAGAUACUGCAACCAGGUU | 1267 | 4983-5005 |
| AD-1736058 | UAGAAUGGUUGUUGAGCUAAA | 999 | 6297-6317 | UUUAGCUCAACAACCAUUCUACA | 1268 | 6295-6317 |
| AD-1736059 | CUGGCCAUCAUUAUUACUGUA | 1000 | 560-580 | UACAGUAAUAAUGAUGGCCAGCU | 1269 | 558-580 |
| AD-1736060 | UUGACAGAUAAAAUACGAAGA | 1001 | 3735-3755 | UCUUCGUAUUUUAUCUGUCAAUU | 1270 | 3733-3755 |
| AD-1736061 | UCACAAACACAUCAUUACAAA | 1002 | 5473-5493 | UUUGUAAUGAUGUGUUUGUGAAU | 1271 | 5471-5493 |
| AD-1736062 | CCAUGAUUGUAUGAAAAUAGA | 1003 | 4962-4982 | UCUAUUUUCAUACAAUCAUGGAA | 1272 | 4960-4982 |
| AD-1736063 | CACAGAGUAAUGAAUAUUUAA | 1004 | 2978-2998 | UUAAAUAUUCAUUACUCUGUGUC | 1273 | 2976-2998 |
| AD-1736064 | AGCUGCUUAGUGGAAGAUGUA | 1005 | 2222-2242 | UACAUCUUCCACUAAGCAGCUCC | 1274 | 2220-2242 |
| AD-1736065 | UAGCUCUGUGUGCUGAUACCA | 1006 | 5606-5626 | UGGUAUCAGCACACAGAGCUAGA | 1275 | 5604-5626 |
| AD-1736066 | CAUUCGAGAGAAUAUGAGCUA | 1007 | 7144-7164 | UAGCUCAUAUUCUCUCGAAUGUA | 1276 | 7142-7164 |
| AD-1736067 | AUGCAUUUUCUGAAAAUGUAA | 1008 | 8734-8754 | UUACAUUUUCAGAAAAUGCAUAC | 1277 | 8732-8754 |
| AD-1736068 | GUUUCGACCAAGUAUCCCAAA | 1009 | 1537-1557 | UUUGGGAUACUUGGUCGAAACUU | 1278 | 1535-1557 |
| AD-1736069 | UCUUGCUUGCCACAAUAUCGA | 1010 | 3610-3630 | UCGAUAUUGUGGCAAGCAAGAUA | 1279 | 3608-3630 |
| AD-1736070 | GGGUACAAGAGGGAAUACAAA | 1011 | 3629-3649 | UUUGUAUUCCCUCUUGUACCCGA | 1280 | 3627-3649 |
| AD-1736071 | GGUGAUCAAAUCCUGUGUCUA | 1012 | 394-414 | UAGACACAGGAUUUGAUCACCUG | 1281 | 392-414 |
| AD-1736072 | CAGUGGGUUUCUAGGAUAGGA | 1013 | 3779-3799 | UCCUAUCCUAGAAACCCACUGUC | 1282 | 3777-3799 |
| AD-1736073 | AACUAAGUAUUUUUAAGGACA | 1014 | 7869-7889 | UGUCCUUAAAAAUACUUAGUUAC | 1283 | 7867-7889 |
| AD-1736074 | CAUAAUGUGAUUGUUAAAUUA | 1015 | 6951-6971 | UAAUUUAACAAUCACAUUAUGGG | 1284 | 6949-6971 |
| AD-1736075 | GAGAAGAAUUAAGAAAACUCA | 1016 | 2820-2840 | UGAGUUUUCUUAAUUCUUCUCAA | 1285 | 2818-2840 |
| AD-1736076 | GAAGAGAAAUUGAUUGUUUAA | 1017 | 7635-7655 | UUAAACAAUCAAUUUCUCUUCUC | 1286 | 7633-7655 |
| AD-1736077 | AGUGUGAAACUUGUGCCAUAA | 1018 | 1218-1238 | UUAUGGCACAAGUUUCACACUUU | 1287 | 1216-1238 |
| AD-1736078 | CUGAGACACUGAGAAAUGUCA | 1019 | 2372-2392 | UGACAUUUCUCAGUGUCUCAGGA | 1288 | 2370-2392 |
| AD-1736079 | UGGCUUAUAUGUGUUUAAAGA | 1020 | 7488-7508 | UCUUUAAACACAUAUAAGCCAAU | 1289 | 7486-7508 |
| AD-1736080 | UGCAUUUUGACAUUGCAAAAA | 1021 | 8188-8208 | UUUUUGCAAUGUCAAAAUGCAAU | 1290 | 8186-8208 |
| AD-1736081 | UUAGGUUAGAAGUUCCAGAAA | 1022 | 5053-5073 | UUUCUGGAACUUCUAACCUAAUG | 1291 | 5051-5073 |
| AD-1736082 | UGCGACAUGAAAACAUCCUUA | 1023 | 951-971 | UAAGGAUGUUUUCAUGUCGCAGC | 1292 | 949-971 |
| AD-1736083 | GAGAGGAUUUUUUACCAUCUA | 1024 | 4095-4115 | UAGAUGGUAAAAAAUCCUCUCUU | 1293 | 4093-4115 |
| AD-1736084 | GAAGGUGAAGAGAAUUUCAAA | 1025 | 8438-8458 | UUUGAAAUUCUCUUCACCUUCCA | 1294 | 8436-8458 |
| AD-1736085 | UGGUAUCUGAAUAUCAUGAAA | 1026 | 1023-1043 | UUUCAUGAUAUUCAGAUACCAGC | 1295 | 1021-1043 |
| AD-1736086 | UGUUGGUUUAAUUUUUCAACA | 1027 | 7658-7678 | UGUUGAAAAAUUAAACCAACAUG | 1296 | 7656-7678 |
| AD-1736087 | UAGUUGUAAUUUAAAUGUGGA | 1028 | 8419-8439 | UCCACAUUUAAAUUACAACUACU | 1297 | 8417-8439 |
| AD-1736088 | GUGAAUGUUUUUGCCAUUUUA | 1029 | 1763-1783 | UAAAAUGGCAAAAACAUUCACAU | 1298 | 1761-1783 |
| AD-1736089 | CAGAGAUGAUUUUUCUUUUAA | 1030 | 6800-6820 | UUAAAAGAAAAAUCAUCUCUGCC | 1299 | 6798-6820 |
| AD-1736090 | AGAAAAUUAGAUUCAGAUCUA | 1031 | 8058-8078 | UAGAUCUGAAUCUAAUUUUCUGU | 1300 | 8056-8078 |
| AD-1736091 | ACUGGAAAAUUAAGAAAGUAA | 1032 | 5390-5410 | UUACUUUCUUAAUUUUCCAGUGG | 1301 | 5388-5410 |
| AD-1736092 | UAAAGUGGGAAAGAUAAUAAA | 1033 | 8762-8782 | UUUAUUAUCUUUCCCACUUUACA | 1302 | 8760-8782 |
| AD-1736093 | CUAGGCUAAGAAAGAGUUGUA | 1034 | 6278-6298 | UACAACUCUUUCUUAGCCUAGUU | 1303 | 6276-6298 |
| AD-1736094 | GCUGAUAAAAUUAAUGGAUAA | 1035 | 6335-6355 | UUAUCCAUUAAUUUUAUCAGCAC | 1304 | 6333-6355 |
| AD-1736095 | CCUGUAUUUCUUUAGUUGUCA | 1036 | 6653-6673 | UGACAACUAAAGAAAUACAGGAA | 1305 | 6651-6673 |
| AD-1736096 | UUUGUUUUGCUUUUGACAAAA | 1037 | 6996-7016 | UUUUGUCAAAAGCAAAACAAAGC | 1306 | 6994-7016 |
| AD-1736097 | UGGCUGUGAAAAUAUUCUCCA | 1038 | 876-896 | UGGAGAAUAUUUUCACAGCCACA | 1307 | 874-896 |
| AD-1736098 | ACUAUUUUAGGCAUAGCACUA | 1039 | 4897-4917 | UAGUGCUAUGCCUAAAAUAGUGC | 1308 | 4895-4917 |
| AD-1736099 | UUUCAGUACUGUAUAAAGUGA | 1040 | 4651-4671 | UCACUUUAUACAGUACUGAAAAU | 1309 | 4649-4671 |
| AD-1736100 | GUGAUGCAAUCUAUGUUUCCA | 1041 | 2129-2149 | UGGAAACAUAGAUUGCAUCACCU | 1310 | 2127-2149 |
| AD-1736101 | UGUUUGGCAGAUCUCAUCAAA | 1042 | 6613-6633 | UUUGAUGAGAUCUGCCAAACAUU | 1311 | 6611-6633 |
| AD-1736102 | AAGGUUGUUUGUGACCAGAAA | 1043 | 1517-1537 | UUUCUGGUCACAAACAACCUUUC | 1312 | 1515-1537 |
| AD-1736103 | GGAAGGAAAUAUUUUGAGAAA | 1044 | 2794-2814 | UUUCUCAAAAUAUUUCCUUCCUU | 1313 | 2792-2814 |
| AD-1736104 | UUUUGGUAGCGUGACUCUUCA | 1045 | 8132-8152 | UGAAGAGUCACGCUACCAAAAGA | 1314 | 8130-8152 |
| AD-1736105 | UGAACAGGAAAAGAUGUAAAA | 1046 | 2045-2065 | UUUUACAUCUUUUCCUGUUCAUU | 1315 | 2043-2065 |
| AD-1736106 | GGGAACCAAGAGGUAUAUGGA | 1047 | 1309-1329 | UCCAUAUACCUCUUGGUUCCCAC | 1316 | 1307-1329 |
| AD-1736107 | UGGCACUAGGUCCAAAUCUUA | 1048 | 2419-2439 | UAAGAUUUGGACCUAGUGCCAGU | 1317 | 2417-2439 |
| AD-1736108 | CACACCUUCAUAUGGAGAUUA | 1049 | 1131-1151 | UAAUCUCCAUAUGAAGGUGUGCC | 1318 | 1129-1151 |
| AD-1736109 | CUGGUUUACUGGGAAAUAGCA | 1050 | 1406-1426 | UGCUAUUUCCCAGUAAACCAGAC | 1319 | 1404-1426 |
| AD-1736110 | GCCCAAAGGAAUGUAUAUAAA | 1051 | 8316-8336 | UUUAUAUACAUUCCUUUGGGCCU | 1320 | 8314-8336 |
| AD-1736111 | UUAACUGCAAGAGUUUACUGA | 1052 | 7265-7285 | UCAGUAAACUCUUGCAGUUAAAA | 1321 | 7263-7285 |
| AD-1736112 | GAACCACUCUCUGAGUGCAAA | 1053 | 677-697 | UUUGCACUCAGAGAGUGGUUCCU | 1322 | 675-697 |
| AD-1736113 | UUAUCUUUUCAUCCUGGCAUA | 1054 | 7607-7627 | UAUGCCAGGAUGAAAAGAUAAAG | 1323 | 7605-7627 |
| AD-1736114 | GUGAGUGUUGGUAUGCCAACA | 1055 | 1605-1625 | UGUUGGCAUACCAACACUCACGC | 1324 | 1603-1625 |
| AD-1736115 | AGAAUGUAUUUGGCUAAAUAA | 1056 | 5338-5358 | UUAUUUAGCCAAAUACAUUCUCA | 1325 | 5336-5358 |
| AD-1736116 | GAACCCUUUUAUUAAGAGGAA | 1057 | 3317-3337 | UUCCUCUUAAUAAAAGGGUUCCA | 1326 | 3315-3337 |
| AD-1736117 | CUCCUGUCCAUAGCUGCGAUA | 1058 | 590-610 | UAUCGCAGCUAUGGACAGGAGGC | 1327 | 588-610 |
| AD-1736118 | ACUGAGUUGAAAUAAGGAAGA | 1059 | 3501-3521 | UCUUCCUUAUUUCAACUCAGUAA | 1328 | 3499-3521 |
| AD-1736119 | CUGCCAAACAGAAGGAGCAUA | 1060 | 337-357 | UAUGCUCCUUCUGUUUGGCAGGU | 1329 | 335-357 |
| AD-1736120 | GGCAAAGUUGUGAAGCACUCA | 1061 | 1563-1583 | UGAGUGCUUCACAACUUUGCCAC | 1330 | 1561-1583 |
| AD-1736121 | AUAGGAUGAUGAAGUUUAGAA | 1062 | 4413-4433 | UUCUAAACUUCAUCAUCCUAUUU | 1331 | 4411-4433 |
| AD-1736122 | AUUCGUAUCUUAAAAUGGCAA | 1063 | 5513-5533 | UUGCCAUUUUAAGAUACGAAUAC | 1332 | 5511-5533 |
| AD-1736123 | CACAGCUUGGGUAAUAGCGUA | 1064 | 3384-3404 | UACGCUAUUACCCAAGCUGUGCU | 1333 | 3382-3404 |
| AD-1736124 | UUUGCAACAACAUAACACUGA | 1065 | 492-512 | UCAGUGUUAUGUUGUUGCAAAAA | 1334 | 490-512 |
| AD-1736125 | UGCCACCAUGUGACUUAUUGA | 1066 | 5153-5173 | UCAAUAAGUCACAUGGUGGCAAA | 1335 | 5151-5173 |
| AD-1736126 | UCGAUAGAGGAAAUGAGAAAA | 1067 | 1499-1519 | UUUUCUCAUUUCCUCUAUCGAGG | 1336 | 1497-1519 |
| AD-1736127 | UUUUUAAGUUAGCAGGACUUA | 1068 | 2152-2172 | UAAGUCCUGCUAACUUAAAAAGG | 1337 | 2150-2172 |
| AD-1736128 | UGUAAAUAAUUAUCUGCCUAA | 1069 | 8696-8716 | UUAGGCAGAUAAUUAUUUACAUC | 1338 | 8694-8716 |
| AD-1736129 | UGGUUGUACGUGCCUCAAAUA | 1070 | 4010-4030 | UAUUUGAGGCACGUACAACCAUA | 1339 | 4008-4030 |

TABLE 5
Modified Sense and Antisense Strand Sequences of ACVRIC dsRNA Agents Comprising anUnsaturated C22 Hydrocarbon Chain
Conjugated to Position 6 on the Sense Strand, Counting from the 5′-end of the Sense Strand

| | | SEQ | | SEQ | | SEQ |
|---|---|---|---|---|---|---|
| Duplex | | ID | | ID | | ID |
| Name | Sense Strand Sequence 5′ to 3′ | NO: | Antisense Sequence 5′ to 3′ | NO: | mRNA Target Sequence | NO: |
| AD-1735861 | ususuuu(Uda)CfaAfAfCfuccauucc | 1340 | VPusAfsggaAfuGfGfaguuUfgAfaaaaasgsa | 1609 | UCUUUUUUCAAACUCCAUUCCUU | 1878 |
| | susa | | | | | |
| AD-1735862 | ascsuuu(Cda)AfuCfUfGfucuucaga | 1341 | VPusAfsucuGfaAfGfacagAfuGfaaagususa | 1610 | UAACUUUCAUCUGUCUUCAGAUG | 1879 |
| | susa | | | | | |
| AD-1735863 | cscsuca(Uda)AfaCfUfUfcuccaguas | 1342 | VPusUfsuacUfgGfAfgaagUfuAfugaggsusg | 1611 | CACCUCAUAACUUCUCCAGUAAU | 1880 |
| | asa | | | | | |
| AD-1735864 | csuscac(Uda)AfuCfUfUfucaacauu | 1343 | VPusUfsaauGfuUfGfaaagAfuAfgugagsgsg | 1612 | CCCUCACUAUCUUUCAACAUUAU | 1881 |
| | sasa | | | | | |
| AD-1735865 | csuscgu(Uda)AfaUfGfCfucucaucc | 1344 | VPusUfsggaUfgAfGfagcaUfuAfacgagsusa | 1613 | UACUCGUUAAUGCUCUCAUCCAC | 1882 |
| | sasa | | | | | |
| AD-1735866 | gsusucu(Uda)CfaUfAfAfuccacuac | 1345 | VPusAfsguaGfuGfGfauuaUfgAfagaacsasg | 1614 | CUGUUCUUCAUAAUCCACUACUG | 1883 |
| | susa | | | | | |
| AD-1735867 | gsusuga(Cda)UfuCfAfUfccaaucuc | 1346 | VPusAfsgagAfuUfGfgaugAfaGfucaacsusc | 1615 | GAGUUGACUUCAUCCAAUCUCUA | 1884 |
| | susa | | | | | |
| AD-1735868 | csusaca(Cda)AfaUfGfAfacuucuua | 1347 | VPusUfsuaaGfaAfGfuucaUfuGfuguagsasa | 1616 | UUCUACACAAUGAACUUCUUAAC | 1885 |
| | sasa | | | | | |
| AD-1735869 | csascau(Cda)UfaGfAfAfuucuuaau | 1348 | VPusAfsauuAfaGfAfauucUfaGfaugugsasc | 1617 | GUCACAUCUAGAAUUCUUAAUUU | 1886 |
| | susa | | | | | |
| AD-1735870 | asasauc(Uda)CfuCfAfUfagcuuucu | 1349 | VPusAfsagaAfaGfCfuaugAfgAfgauuuscsu | 1618 | AGAAAUCUCUCAUAGCUUUCUUU | 1887 |
| | susa | | | | | |
| AD-1735871 | cscsuac(Uda)AfuUfGfUfagaauuac | 1350 | VPusAfsguaAfuUfCfuacaAfuAfguaggsusg | 1619 | CACCUACUAUUGUAGAAUUACUA | 1888 |
| | susa | | | | | |
| AD-1735872 | asuscuu(Cda)UfuUfUfAfgugcauua | 1351 | VPusUfsuaaUfgCfAfcuaaAfaGfaagausgsa | 1620 | UCAUCUUCUUUUAGUGCAUUAAA | 1889 |
| | sasa | | | | | |
| AD-1735873 | csuscau(Gda)UfaCfUfCfuucugauu | 1352 | VPusGfsaauCfaGfAfagagUfaCfaugagscsu | 1621 | AGCUCAUGUACUCUUCUGAUUCU | 1890 |
| | scsa | | | | | |
| AD-1735874 | ususucu(Cda)UfuUfGfUfaccuugga | 1353 | VPusUfsuccAfaGfGfuacaAfaGfagaaasgsa | 1622 | UCUUUCUCUUUGUACCUUGGAAU | 1891 |
| | sasa | | | | | |
| AD-1735875 | usgsgcu(Uda)AfuAfCfAfucuucaga | 1354 | VPusUfsucuGfaAfGfauguAfuAfagccasgsa | 1623 | UCUGGCUUAUACAUCUUCAGAAA | 1892 |
| | sasa | | | | | |
| AD-1735876 | csasaaa(Cda)AfaAfAfCfuacuccuas | 1355 | VPusAfsuagGfaGfUfaguuUfuGfuuuugsusu | 1624 | AACAAAACAAAACUACUCCUAUU | 1893 |
| | usa | | | | | |
| AD-1735877 | ascsuag(Cda)AfgAfAfCfucuuauga | 1356 | VPusUfsucaUfaAfGfaguuCfuGfcuagusasa | 1625 | UUACUAGCAGAACUCUUAUGAAA | 1894 |
| | sasa | | | | | |
| AD-1735878 | cscsaag(Uda)CfaCfUfCfuauaauucs | 1357 | VPusGfsgaaUfuAfUfagagUfgAfcuuggsgsa | 1626 | UCCCAAGUCACUCUAUAAUUCCU | 1895 |
| | csa | | | | | |
| AD-1735879 | uscscau(Uda)UfuUfCfUfugucauua | 1358 | VPusAfsuaaUfgAfCfaagaAfaAfauggasgsa | 1627 | UCUCCAUUUUUCUUGUCAUUAUG | 1896 |
| | susa | | | | | |
| AD-1735880 | csasaca(Cda)CfuCfAfAfcucaucuus | 1359 | VPusAfsaagAfuGfAfguugAfgGfuguugscsu | 1628 | AGCAACACCUCAACUCAUCUUUU | 1897 |
| | usa | | | | | |
| AD-1735881 | ascsucu(Gda)AfaAfGfAfucugauuu | 1360 | VPusUfsaaaUfcAfGfaucuUfuCfagagususu | 1629 | AAACUCUGAAAGAUCUGAUUUAU | 1898 |
| | sasa | | | | | |
| AD-1735882 | csusagu(Uda)CfuUfUfUfccgcaaug | 1361 | VPusUfscauUfgCfGfgaaaAfgAfacuagsasa | 1630 | UUCUAGUUCUUUUCCGCAAUGAC | 1899 |
| | sasa | | | | | |
| AD-1735883 | csusuuc(Ada)CfuCfUfGfaagaaauc | 1362 | VPusGfsgauUfuCfUfucagAfgUfgaaagsusu | 1631 | AACUUUCACUCUGAAGAAAUCCG | 1900 |
| | scsa | | | | | |
| AD-1735884 | uscsuaa(Cda)UfuUfUfCfucucuuuc | 1363 | VPusAfsgaaAfgAfGfagaaAfaGfuuagasusa | 1632 | UAUCUAACUUUUCUCUCUUUCUC | 1901 |
| | susa | | | | | |
| AD-1735885 | uscsuca(Ada)CfuUfUfGfugucaaag | 1364 | VPusUfscuuUfgAfCfacaaAfgUfugagasusa | 1633 | UAUCUCAACUUUGUGUCAAAGAA | 1902 |
| | sasa | | | | | |
| AD-1735886 | csusuca(Ada)UfaAfUfCfauuccuuu | 1365 | VPusUfsaaaGfgAfAfugauUfaUfugaagsasc | 1634 | GUCUUCAAUAAUCAUUCCUUUAA | 1903 |
| | sasa | | | | | |
| AD-1735887 | usasacu(Gda)CfuCfUfUfcguauuaa | 1366 | VPusCfsuuaAfuAfCfgaagAfgCfaguuasgsg | 1635 | CCUAACUGCUCUUCGUAUUAAGA | 1904 |
| | sgsa | | | | | |
| AD-1735888 | uscsuuc(Uda)GfuCfAfUfaguuccaa | 1367 | VPusGfsuugGfaAfCfuaugAfcAfgaagascsu | 1636 | AGUCUUCUGUCAUAGUUCCAACA | 1905 |
| | scsa | | | | | |
| AD-1735889 | asuscuc(Ada)GfaAfCfCfauaucugu | 1368 | VPusAfsacaGfaUfAfugguUfcUfgagaususu | 1637 | AAAUCUCAGAACCAUAUCUGUUG | 1906 |
| | susa | | | | | |
| AD-1735890 | usgsaau(Cda)UfaUfCfUfucauuuua | 1369 | VPusGfsuaaAfaUfGfaagaUfaGfauucasgsu | 1638 | ACUGAAUCUAUCUUCAUUUUACU | 1907 |
| | scsa | | | | | |
| AD-1735891 | ascscac(Uda)AfaAfCfUfuguuccuu | 1370 | VPusAfsaagGfaAfCfaaguUfuAfguggususc | 1639 | GAACCACUAAACUUGUUCCUUUC | 1908 |
| | susa | | | | | |
| AD-1735892 | asgsugu(Uda)CfuUfUfAfcuccuuac | 1371 | VPusUfsguaAfgGfAfguaaAfgAfacacusgsa | 1640 | UCAGUGUUCUUUACUCCUUACAG | 1909 |
| | sasa | | | | | |
| AD-1735893 | csusuua(Cda)UfcUfUfAfacaggauu | 1372 | VPusUfsaauCfcUfGfuuaaGfaGfuaaagsgsa | 1641 | UCCUUUACUCUUAACAGGAUUAU | 1910 |
| | sasa | | | | | |
| AD-1735894 | usasuac(Cda)UfaAfGfAfacauauua | 1373 | VPusGfsuaaUfaUfGfuucuUfaGfguauasasg | 1642 | CUUAUACCUAAGAACAUAUUACA | 1911 |
| | scsa | | | | | |
| AD-1735895 | csusucu(Ada)CfuGfAfGfaugaucca | 1374 | VPusUfsuggAfuCfAfucucAfgUfagaagsasa | 1643 | UUCUUCUACUGAGAUGAUCCAAG | 1912 |
| | sasa | | | | | |
| AD-1735896 | csusgua(Uda)UfuUfCfUfcauagagu | 1375 | VPusUfsacuCfuAfUfgagaAfaAfuacagsasa | 1644 | UUCUGUAUUUUCUCAUAGAGUAC | 1913 |
| | sasa | | | | | |
| AD-1735897 | ascsuuu(Uda)CfuCfUfUfucaguugu | 1376 | VPusUfsacaAfcUfGfaaagAfgAfaaagusasu | 1645 | AUACUUUUCUCUUUCAGUUGUAG | 1914 |
| | sasa | | | | | |
| AD-1735898 | asusuaa(Uda)UfuCfUfAfgucuguga | 1377 | VPusUfsucaCfaGfAfcuagAfaAfuuaausgsa | 1646 | UCAUUAAUUUCUAGUCUGUGAAA | 1915 |
| | sasa | | | | | |
| AD-1735899 | csuscug(Uda)AfuAfAfGfagguuuca | 1378 | VPusGfsugaAfaCfCfucuuAfuAfcagagsasa | 1647 | UUCUCUGUAUAAGAGGUUUCACC | 1916 |
| | scsa | | | | | |
| AD-1735900 | gsascuu(Uda)CfaAfAfGfuacuaaua | 1379 | VPusGfsuauUfaGfUfacuuUfgAfaagucsasa | 1648 | UUGACUUUCAAAGUACUAAUACU | 1917 |
| | scsa | | | | | |
| AD-1735901 | ususugc(Cda)AfuUfAfUfacaaaguu | 1380 | VPusAfsaacUfuUfGfuauaAfuGfgcaaascsa | 1649 | UGUUUGCCAUUAUACAAAGUUUG | 1918 |
| | susa | | | | | |
| AD-1735902 | gsasaca(Cda)UfaUfCfGfacauaccus | 1381 | VPusGfsaggUfaUfGfucgaUfaGfuguucsasg | 1650 | CUGAACACUAUCGACAUACCUCA | 1919 |
| | csa | | | | | |
| AD-1735903 | usasgaa(Uda)GfaCfAfUfuuacuaau | 1382 | VPusUfsauuAfgUfAfaaugUfcAfuucuascsg | 1651 | CGUAGAAUGACAUUUACUAAUAU | 1920 |
| | sasa | | | | | |
| AD-1735904 | asasgcu(Ada)CfuUfAfGfaaauguuu | 1383 | VPusUfsaaaCfaUfUfucuaAfgUfagcuusgsu | 1652 | ACAAGCUACUUAGAAAUGUUUAG | 1921 |
| | sasa | | | | | |
| AD-1735905 | cscsuua(Gda)UfaCfUfCfucagagau | 1384 | VPusAfsaucUfcUfGfagagUfaCfuaaggsgsa | 1653 | UCCCUUAGUACUCUCAGAGAUUA | 1922 |
| | susa | | | | | |
| AD-1735906 | usgsaau(Gda)CfaCfAfCfuaacguaas | 1385 | VPusAfsuuaCfgUfUfagugUfgCfauucasasg | 1654 | CUUGAAUGCACACUAACGUAAUG | 923 |
| | usa | | | | | |
| AD-1735907 | asasgcc(Uda)AfaUfGfAfugauaauu | 1386 | VPusUfsaauUfaUfCfaucaUfuAfggcuususg | 1655 | CAAAGCCUAAUGAUGAUAAUUAU | 924 |
| | sasa | | | | | |
| AD-1735908 | asasacu(Uda)UfuUfAfCfuaucccau | 1387 | VPusUfsaugGfgAfUfaguaAfaAfaguuuscsc | 1656 | GGAAACUUUUUACUAUCCCAUAU | 1925 |
| | sasa | | | | | |
| AD-1735909 | csusagc(Uda)GfaUfAfCfucuuaagu | 1388 | VPusUfsacuUfaAfGfaguaUfcAfgcuagscsu | 1657 | AGCUAGCUGAUACUCUUAAGUAU | 1926 |
| | sasa | | | | | |
| AD-1735910 | asgscau(Gda)AfaAfAfGfauaacucu | 1389 | VPusUfsagaGfuUfAfucuuUfuCfaugcusgsc | 1658 | GCAGCAUGAAAAGAUAACUCUAA | 1927 |
| | sasa | | | | | |
| AD-1735911 | usasagc(Cda)AfuUfAfUfgcuauuag | 1390 | VPusAfscuaAfuAfGfcauaAfuGfgcuuasgsu | 1659 | ACUAAGCCAUUAUGCUAUUAGUU | 1928 |
| | susa | | | | | |
| AD-1735912 | uscsuag(Cda)UfuUfUfAfgcuaacau | 1391 | VPusUfsaugUfuAfGfcuaaAfaGfcuagasasu | 1660 | AUUCUAGCUUUUAGCUAACAUAU | 1929 |
| | sasa | | | | | |
| AD-1735913 | gsusgug(Ada)UfuCfUfUfcaaacuuc | 1392 | VPusUfsgaaGfuUfUfgaagAfaUfcacacsasa | 1661 | UUGUGUGAUUCUUCAAACUUCAC | 1930 |
| | sasa | | | | | |
| AD-1735914 | uscsucc(Ada)GfaCfCfUfaacaguuu | 1393 | VPusAfsaaaCfuGfUfuaggUfcUfggagasgsa | 1662 | UCUCUCCAGACCUAACAGUUUUA | 1931 |
| | susa | | | | | |
| AD-1735915 | csusuuu(Uda)UfuCfAfUfcuagccuu | 1394 | VPusCfsaagGfcUfAfgaugAfaAfaaaagsasa | 1663 | UUCUUUUUUUCAUCUAGCCUUGC | 1932 |
| | sgsa | | | | | |
| AD-1735916 | csasaaa(Uda)AfaUfUfCfuugacaucs | 1395 | VPusAfsgauGfuCfAfagaaUfuAfuuuugsasc | 1664 | GUCAAAAUAAUUCUUGACAUCUA | 1933 |
| | usa | | | | | |
| AD-1735917 | gscsauc(Ada)CfuUfUfCfugaaaaua | 1396 | VPusUfsuauUfuUfCfagaaAfgUfgaugcsusg | 1665 | CAGCAUCACUUUCUGAAAAUAAG | 1934 |
| | sasa | | | | | |
| AD-1735918 | uscsuuu(Uda)GfuGfAfAfacauacua | 1397 | VPusAfsuagUfaUfGfuuucAfcAfaaagasusg | 1666 | CAUCUUUUGUGAAACAUACUAUU | 1935 |
| | susa | | | | | |
| AD-1735919 | csusauu(Uda)CfuUfCfCfauaggcua | 1398 | VPusUfsuagCfcUfAfuggaAfgAfaauagsasa | 1667 | UUCUAUUUCUUCCAUAGGCUAAA | 1936 |
| | sasa | | | | | |
| AD-1735920 | csusuca(Ada)UfgAfAfGfuguuaaca | 1399 | VPusUfsuguUfaAfCfacuuCfaUfugaagsasg | 1668 | CUCUUCAAUGAAGUGUUAACAAC | 1937 |
| | sasa | | | | | |
| AD-1735921 | ususugc(Cda)UfuCfAfUfucuacuuu | 1400 | VPusGfsaaaGfuAfGfaaugAfaGfgcaaasasc | 1669 | GUUUUGCCUUCAUUCUACUUUCU | 1938 |
| | scsa | | | | | |
| AD-1735922 | asgsguu(Uda)UfuAfAfAfuguccuaa | 1401 | VPusUfsuuaGfgAfCfauuuAfaAfaaccusgsa | 1670 | UCAGGUUUUUAAAUGUCCUAAAA | 1939 |
| | sasa | | | | | |
| AD-1735923 | gsasaua(Uda)CfuUfUfGfaguccuuc | 1402 | VPusUfsgaaGfgAfCfucaaAfgAfuauucsasc | 1671 | GUGAAUAUCUUUGAGUCCUUCAA | 1940 |
| | sasa | | | | | |
| AD-1735924 | gsasaca(Cda)UfuCfCfAfaagauuaas | 1403 | VPusAfsuuaAfuCfUfuuggAfaGfuguucsasg | 1672 | CUGAACACUUCCAAAGAUUAAUC | 1941 |
| | usa | | | | | |
| AD-1735925 | csusaaa(Uda)UfuUfGfUfcuaacaaas | 1404 | VPusAfsuuuGfuUfAfgacaAfaAfuuuagsgsc | 1673 | GCCUAAAUUUUGUCUAACAAAUG | 1942 |
| | usa | | | | | |
| AD-1735926 | gsusaaa(Cda)AfaCfUfUfaaaauugcs | 1405 | VPusAfsgcaAfuUfUfuaagUfuGfuuuacsusc | 1674 | GAGUAAACAACUUAAAAUUGCUU | 1943 |
| | usa | | | | | |
| AD-1735927 | asasaau(Ada)AfuUfUfAfcucacuca | 1406 | VPusCfsugaGfuGfAfguaaAfuUfauuuusasg | 1675 | CUAAAAUAAUUUACUCACUCAGA | 1944 |
| | sgsa | | | | | |
| AD-1735928 | gsascau(Cda)UfaUfUfCfuguugguc | 1407 | VPusAfsgacCfaAfCfagaaUfaGfaugucsasg | 1676 | CUGACAUCUAUUCUGUUGGUCUG | 1945 |
| | susa | | | | | |
| AD-1735929 | usasacu(Gda)AfgUfAfGfucuuauau | 1408 | VPusAfsauaUfaAfGfacuaCfuCfaguuasgsg | 1677 | CCUAACUGAGUAGUCUUAUAUUU | 1946 |
| | susa | | | | | |
| AD-1735930 | uscsugg(Ada)AfuUfUfAfgccuucua | 1409 | VPusUfsuagAfaGfGfcuaaAfuUfccagascsg | 1678 | CGUCUGGAAUUUAGCCUUCUAAU | 1947 |
| | sasa | | | | | |
| AD-1735931 | ascsaug(Cda)CfuUfGfAfacucuuga | 1410 | VPusUfsucaAfgAfGfuucaAfgGfcaugususu | 1679 | AAACAUGCCUUGAACUCUUGAAC | 1948 |
| | sasa | | | | | |
| AD-1735932 | asuscaa(Gda)CfaCfUfUfcuugcacus | 1411 | VPusAfsaguGfcAfAfgaagUfgCfuugaususu | 1680 | AAAUCAAGCACUUCUUGCACUUG | 1949 |
| | usa | | | | | |
| AD-1735933 | csusugc(Uda)UfuUfCfAfucuuuaua | 1412 | VPusGfsuauAfaAfGfaugaAfaAfgcaagsusg | 1681 | CACUUGCUUUUCAUCUUUAUACU | 1950 |
| | scsa | | | | | |
| AD-1735934 | ususuuc(Uda)CfcCfUfAfuguaauau | 1413 | VPusGfsauaUfuAfCfauagGfgAfgaaaasgsc | 1682 | GCUUUUCUCCCUAUGUAAUAUCU | 1951 |
| | scsa | | | | | |
| AD-1735935 | usgscuu(Uda)CfuUfCfUfaugucuaa | 1414 | VPusUfsuuaGfaCfAfuagaAfgAfaagcasusa | 1683 | UAUGCUUUCUUCUAUGUCUAAAA | 1952 |
| | sasa | | | | | |
| AD-1735936 | ascscug(Cda)UfuAfGfCfaaaucguc | 1415 | VPusAfsgacGfaUfUfugcuAfaGfcaggususc | 1684 | GAACCUGCUUAGCAAAUCGUCUG | 1953 |
| | susa | | | | | |
| AD-1735937 | asusauu(Cda)CfuCfUfAfcccuuuca | 1416 | VPusGfsugaAfaGfGfguagAfgGfaauauscsu | 1685 | AGAUAUUCCUCUACCCUUUCACA | 1954 |
| | scsa | | | | | |
| AD-1735938 | uscscca(Cda)UfuUfUfCfuuuggguu | 1417 | VPusAfsaacCfcAfAfagaaAfaGfugggasusu | 1686 | AAUCCCACUUUUCUUUGGGUUUC | 1955 |
| | susa | | | | | |
| AD-1735939 | asgsgcu(Uda)CfuUfGfUfauauacuu | 1418 | VPusUfsaagUfaUfAfuacaAfgAfagccusgsc | 1687 | GCAGGCUUCUUGUAUAUACUUAU | 1956 |
| | sasa | | | | | |
| AD-1735940 | asasacu(Uda)GfaUfCfUfguguuuca | 1419 | VPusCfsugaAfaCfAfcagaUfcAfaguuusgsa | 1688 | UCAAACUUGAUCUGUGUUUCAGG | 1957 |
| | sgsa | | | | | |
| AD-1735941 | ususugc(Uda)UfuAfAfUfgcuaaauu | 1420 | VPusGfsaauUfuAfGfcauuAfaAfgcaaasasg | 1689 | CUUUUGCUUUAAUGCUAAAUUCC | 1958 |
| | scsa | | | | | |
| AD-1735942 | asgsacu(Ada)GfcUfGfUfuauuugua | 1421 | VPusAfsuacAfaAfUfaacaGfcUfagucususu | 1690 | AAAGACUAGCUGUUAUUUGUAUU | 1959 |
| | susa | | | | | |
| AD-1735943 | ususccu(Ada)CfuUfUfCfccuugaau | 1422 | VPusUfsauuCfaAfGfggaaAfgUfaggaasgsc | 1691 | GCUUCCUACUUUCCCUUGAAUAG | 1960 |
| | sasa | | | | | |
| AD-1735944 | csasgaa(Cda)UfgAfAfUfgcucaagu | 1423 | VPusGfsacuUfgAfGfcauuCfaGfuucugsgsa | 1692 | UCCAGAACUGAAUGCUCAAGUCU | 1961 |
| | scsa | | | | | |
| AD-1735945 | uscsuug(Uda)UfaCfUfAfauuucuca | 1424 | VPusAfsugaGfaAfAfuuagUfaAfcaagasasc | 1693 | GUUCUUGUUACUAAUUUCUCAUA | 1962 |
| | susa | | | | | |
| AD-1735946 | csuscag(Uda)GfaUfAfGfccuuuaua | 1425 | VPusGfsuauAfaAfGfgcuaUfcAfcugagsusu | 1694 | AACUCAGUGAUAGCCUUUAUACC | 1963 |
| | scsa | | | | | |
| AD-1735947 | ususuua(Uda)GfcUfCfCfuaaaacau | 1426 | VPusGfsaugUfuUfUfaggaGfcAfuaaaasgsa | 1695 | UCUUUUAUGCUCCUAAAACAUCU | 1964 |
| | scsa | | | | | |
| AD-1735948 | ususcuc(Cda)UfuAfGfGfuuauguuc | 1427 | VPusUfsgaaCfaUfAfaccuAfaGfgagaasusu | 1696 | AAUUCUCCUUAGGUUAUGUUCAG | 1965 |
| | sasa | | | | | |
| AD-1735949 | asusugc(Uda)CfaUfCfGfagacauaas | 1428 | VPusUfsuuaUfgUfCfucgaUfgAfgcaausasg | 1697 | CUAUUGCUCAUCGAGACAUAAAA | 1966 |
| | asa | | | | | |
| AD-1735950 | cscsuuu(Gda)UfaCfAfUfacacacuu | 1429 | VPusUfsaagUfgUfGfuaugUfaCfaaaggsasa | 1698 | UUCCUUUGUACAUACACACUUAG | 1967 |
| | sasa | | | | | |
| AD-1735951 | ususgaa(Cda)CfaAfGfAfgcacauga | 1430 | VPusUfsucaUfgUfGfcucuUfgGfuucaasgsu | 1699 | ACUUGAACCAAGAGCACAUGAAU | 1968 |
| | sasa | | | | | |
| AD-1735952 | uscsagg(Gda)AfuUfUfUfaaagucua | 1431 | VPusUfsuagAfcUfUfuaaaAfuCfccugasgsg | 1700 | CCUCAGGGAUUUUAAAGUCUAAU | 1969 |
| | sasa | | | | | |
| AD-1735953 | usascuu(Gda)CfuUfAfUfaaagugau | 1432 | VPusUfsaucAfcUfUfuauaAfgCfaaguascsc | 1701 | GGUACUUGCUUAUAAAGUGAUAG | 1970 |
| | sasa | | | | | |
| AD-1735954 | asusgca(Uda)CfuAfAfAfccuaccuu | 1433 | VPusCfsaagGfuAfGfguuuAfgAfugcausgsc | 1702 | GCAUGCAUCUAAACCUACCUUGA | 1971 |
| | sgsa | | | | | |
| AD-1735955 | ususggg(Ada)AfaAfCfAfcuauuaug | 1434 | VPusUfscauAfaUfAfguguUfuUfcccaasgsu | 1703 | ACUUGGGAAAACACUAUUAUGAA | 1972 |
| | sasa | | | | | |
| AD-1735956 | asasacu(Cda)CfaAfAfGfagaaaugas | 1435 | VPusUfsucaUfuUfCfucuuUfgGfaguuusgsa | 1704 | UCAAACUCCAAAGAGAAAUGAAU | 1973 |
| | asa | | | | | |
| AD-1735957 | asusggg(Cda)AfuUfUfUfucaaaaca | 1436 | VPusAfsuguUfuUfGfaaaaAfuGfcccausasu | 1705 | AUAUGGGCAUUUUUCAAAACAUU | 1974 |
| | susa | | | | | |
| AD-1735958 | asusaau(Gda)GfaAfCfUfuggacuca | 1437 | VPusUfsugaGfuCfCfaaguUfcCfauuauscsu | 1706 | AGAUAAUGGAACUUGGACUCAAC | 1975 |
| | sasa | | | | | |
| AD-1735959 | usgsugu(Ada)GfaUfGfAfcgaaacca | 1438 | VPusUfsuggUfuUfCfgucaUfcUfacacasasa | 1707 | UUUGUGUAGAUGACGAAACCAAG | 1976 |
| | sasa | | | | | |
| AD-1735960 | ususcua(Cda)CfuCfAfAfagauaaga | 1439 | VPusGfsucuUfaUfCfuuugAfgGfuagaascsa | 1708 | UGUUCUACCUCAAAGAUAAGACA | 1977 |
| | scsa | | | | | |
| AD-1735961 | asgsaag(Gda)CfaUfAfUfugaguuau | 1440 | VPusAfsauaAfcUfCfaauaUfgCfcuucusasa | 1709 | UUAGAAGGCAUAUUGAGUUAUUU | 1978 |
| | susa | | | | | |
| AD-1735962 | asusgga(Gda)UfuUfUfUfcugcuauu | 1441 | VPusAfsaauAfgCfAfgaaaAfaCfuccausgsu | 1710 | ACAUGGAGUUUUUCUGCUAUUUU | 1979 |
| | susa | | | | | |
| AD-1735963 | uscsagu(Uda)UfcUfCfAfuuaguuug | 1442 | VPusAfscaaAfcUfAfaugaGfaAfacugasgsg | 1711 | CCUCAGUUUCUCAUUAGUUUGUC | 1980 |
| | susa | | | | | |
| AD-1735964 | csusgca(Uda)AfuAfUfAfagaacgaa | 1443 | VPusUfsuucGfuUfCfuuauAfuAfugcagsasa | 1712 | UUCUGCAUAUAUAAGAACGAAAU | 1981 |
| | sasa | | | | | |
| AD-1735965 | usgsagc(Cda)AfaAfAfUfauuaaauu | 1444 | VPusGfsaauUfuAfAfuauuUfuGfgcucasgsa | 1713 | UCUGAGCCAAAAUAUUAAAUUCU | 1982 |
| | scsa | | | | | |
| AD-1735966 | asgsaau(Gda)AfuAfUfUfuccuguuu | 1445 | VPusAfsaaaCfaGfGfaaauAfuCfauucususg | 1714 | CAAGAAUGAUAUUUCCUGUUUUA | 1983 |
| | susa | | | | | |
| AD-1735967 | gsusgcu(Cda)UfaUfAfUfaaaucuuc | 1446 | VPusGfsgaaGfaUfUfuauaUfaGfagcacsasa | 1715 | UUGUGCUCUAUAUAAAUCUUCCC | 1984 |
| | scsa | | | | | |
| AD-1735968 | ususuac(Ada)GfaGfAfAfgcaucuua | 1447 | VPusAfsuaaGfaUfGfcuucUfcUfguaaasasu | 1716 | AUUUUACAGAGAAGCAUCUUAUU | 1985 |
| | susa | | | | | |
| AD-1735969 | gsuscuu(Gda)AfgUfUfCfugcaugac | 1448 | VPusUfsgucAfuGfCfagaaCfuCfaagacsasu | 1717 | AUGUCUUGAGUUCUGCAUGACAG | 1986 |
| | sasa | | | | | |
| AD-1735970 | csusuca(Ada)AfuCfUfAfuaauuuua | 1449 | VPusGfsuaaAfaUfUfauagAfuUfugaagsgsg | 1718 | CCCUUCAAAUCUAUAAUUUUACU | 1987 |
| | scsa | | | | | |
| AD-1735971 | gsasagc(Ada)UfaUfAfGfaacucuau | 1450 | VPusAfsauaGfaGfUfucuaUfaUfgcuucsasc | 1719 | GUGAAGCAUAUAGAACUCUAUUU | 1988 |
| | susa | | | | | |
| AD-1735972 | gsasgcu(Gda)CfuUfCfUfaaauaacas | 1451 | VPusUfsuguUfaUfUfuagaAfgCfagcucsusc | 1720 | GAGAGCUGCUUCUAAAUAACAAA | 1989 |
| | asa | | | | | |
| AD-1735973 | usgsacu(Gda)UfaUfUfUfccugauca | 1452 | VPusAfsugaUfcAfGfgaaaUfaCfagucascsa | 1721 | UGUGACUGUAUUUCCUGAUCAUU | 1990 |
| | susa | | | | | |
| AD-1735974 | gsusucu(Gda)AfaGfGfAfuauacuuc | 1453 | VPusAfsgaaGfuAfUfauccUfuCfagaacsgsc | 1722 | GCGUUCUGAAGGAUAUACUUCUG | 1991 |
| | susa | | | | | |
| AD-1735975 | gsusgaa(Gda)CfaUfGfAfuucaauac | 1454 | VPusAfsguaUfuGfAfaucaUfgCfuucacsasg | 1723 | CUGUGAAGCAUGAUUCAAUACUG | 1992 |
| | susa | | | | | |
| AD-1735976 | asasugc(Uda)GfcUfUfCfacagauuu | 1455 | VPusAfsaaaUfcUfGfugaaGfcAfgcauuscsg | 1724 | CGAAUGCUGCUUCACAGAUUUUU | 1993 |
| | susa | | | | | |
| AD-1735977 | usgsugg(Gda)UfuAfUfGfuuaaucu | 1456 | VPusUfscagAfuUfAfacauAfaCfccacasgsu | 1725 | ACUGUGGGUUAUGUUAAUCUGAA | 1994 |
| | gsasa | | | | | |
| AD-1735978 | gsasgua(Cda)AfgAfAfGfaaugcuca | 1457 | VPusAfsugaGfcAfUfucuuCfuGfuacucsasc | 1726 | GUGAGUACAGAAGAAUGCUCAUG | 1995 |
| | susa | | | | | |
| AD-1735979 | csusgua(Uda)UfuCfAfUfccuagauu | 1458 | VPusAfsaauCfuAfGfgaugAfaAfuacagscsu | 1727 | AGCUGUAUUUCAUCCUAGAUUUU | 1996 |
| | susa | | | | | |
| AD-1735980 | asasucu(Gda)GfuAfAfAfugcuggaa | 1459 | VPusUfsuucCfaGfCfauuuAfcCfagauusgsc | 1728 | GCAAUCUGGUAAAUGCUGGAAAA | 1997 |
| | sasa | | | | | |
| AD-1735981 | usgscuc(Ada)GfaUfUfAfccugaucg | 1460 | VPusAfscgaUfcAfGfguaaUfcUfgagcasasu | 1729 | AUUGCUCAGAUUACCUGAUCGUG | 1998 |
| | susa | | | | | |
| AD-1735982 | gsascag(Cda)UfaUfGfGfaguuugcg | 1461 | VPusAfscgcAfaAfCfuccaUfaGfcugucsasg | 1730 | CUGACAGCUAUGGAGUUUGCGUG | 1999 |
| | susa | | | | | |
| AD-1735983 | usasgaa(Cda)AfuGfCfUfcacuuacas | 1462 | VPusUfsuguAfaGfUfgagcAfuGfuucuasusa | 1731 | UAUAGAACAUGCUCACUUACAAA | 2000 |
| | asa | | | | | |
| AD-1735984 | asusuau(Gda)CfuUfCfAfuuucacua | 1463 | VPusAfsuagUfgAfAfaugaAfgCfauaauscsa | 1732 | UGAUUAUGCUUCAUUUCACUAUG | 2001 |
| | susa | | | | | |
| AD-1735985 | csasaca(Gda)CfaAfGfUfcauaaaags | 1464 | VPusAfscuuUfuAfUfgacuUfgCfuguugsgsu | 1733 | ACCAACAGCAAGUCAUAAAAGUA | 2002 |
| | usa | | | | | |
| AD-1735986 | ususguc(Uda)GfaAfAfAfugucuuua | 1465 | VPusUfsuaaAfgAfCfauuuUfcAfgacaasusg | 1734 | CAUUGUCUGAAAAUGUCUUUAAG | 2003 |
| | sasa | | | | | |
| AD-1735987 | usgsuug(Gda)UfuCfAfAfaggacaau | 1466 | VPusAfsauuGfuCfCfuuugAfaCfcaacasgsa | 1735 | UCUGUUGGUUCAAAGGACAAUUG | 2004 |
| | susa | | | | | |
| AD-1735988 | uscsuaa(Ada)CfuUfUfAfauauaucg | 1467 | VPusUfscgaUfaUfAfuuaaAfgUfuuagasgsc | 1736 | GCUCUAAACUUUAAUAUAUCGAA | 2005 |
| | sasa | | | | | |
| AD-1735989 | asasgug(Ada)AfaCfAfUfgacugucg | 1468 | VPusAfscgaCfaGfUfcaugUfuUfcacuusasa | 1737 | UUAAGUGAAACAUGACUGUCGUG | 2006 |
| | susa | | | | | |
| AD-1735990 | asuscgu(Cda)CfaUfAfCfaguuuuca | 1469 | VPusAfsugaAfaAfCfuguaUfgGfacgausasu | 1738 | AUAUCGUCCAUACAGUUUUCAUU | 2007 |
| | susa | | | | | |
| AD-1735991 | csusuca(Gda)GfaAfAfUfaguaggaa | 1470 | VPusUfsuucCfuAfCfuauuUfcCfugaagscsa | 1739 | UGCUUCAGGAAAUAGUAGGAAAA | 2008 |
| | sasa | | | | | |
| AD-1735992 | csasccc(Uda)UfuCfUfGfuaaggacu | 1471 | VPusCfsaguCfcUfUfacagAfaAfgggugsgsg | 1740 | CCCACCCUUUCUGUAAGGACUGU | 2009 |
| | sgsa | | | | | |
| AD-1735993 | asasaga(Gda)AfuAfCfCfugacauccs | 1472 | VPusAfsggaUfgUfCfagguAfuCfucuuususg | 1741 | CAAAAGAGAUACCUGACAUCCUG | 2010 |
| | usa | | | | | |
| AD-1735994 | asusgag(Cda)AfuUfAfAfuguuuucu | 1473 | VPusCfsagaAfaAfCfauuaAfuGfcucaususa | 1742 | UAAUGAGCAUUAAUGUUUUCUGA | 2011 |
| | sgsa | | | | | |
| AD-1735995 | usgsaca(Uda)UfuUfUfUfcucauaga | 1474 | VPusUfsucuAfuGfAfgaaaAfaAfugucasasc | 1743 | GUUGACAUUUUUUCUCAUAGAAA | 2012 |
| | sasa | | | | | |
| AD-1735996 | csasgag(Ada)UfuAfAfAfugacuacu | 1475 | VPusUfsaguAfgUfCfauuuAfaUfcucugsasu | 1744 | AUCAGAGAUUAAAUGACUACUAG | 2013 |
| | sasa | | | | | |
| AD-1735997 | gscsugu(Cda)AfaAfUfGfuuauauug | 1476 | VPusAfscaaUfaUfAfacauUfuGfacagcsasu | 1745 | AUGCUGUCAAAUGUUAUAUUGUU | 2014 |
| | susa | | | | | |
| AD-1735998 | asasuau(Ada)AfuAfCfUfacagcaaas | 1477 | VPusUfsuuuGfcUfGfuaguAfuUfauauuscsc | 1746 | GGAAUAUAAUACUACAGCAAAAU | 2015 |
| | asa | | | | | |
| AD-1735999 | gsgsugu(Cda)UfaAfAfUfauaauuuc | 1478 | VPusUfsgaaAfuUfAfuauuUfaGfacaccscsu | 1747 | AGGGUGUCUAAAUAUAAUUUCAU | 2016 |
| | sasa | | | | | |
| AD-1736000 | csuscuu(Uda)AfuAfCfAfaaaugaga | 1479 | VPusCfsucuCfaUfUfuuguAfuAfaagagsasa | 1748 | UUCUCUUUAUACAAAAUGAGAGU | 2017 |
| | sgsa | | | | | |
| AD-1736001 | gscsagu(Ada)CfcAfAfAfugaauaaa | 1480 | VPusUfsuuuAfuUfCfauuuGfgUfacugcsusa | 1749 | UAGCAGUACCAAAUGAAUAAAAG | 2018 |
| | sasa | | | | | |
| AD-1736002 | ususguu(Gda)GfuAfCfAfcaagguaa | 1481 | VPusUfsuuaCfcUfUfguguAfcCfaacaasusc | 1750 | GAUUGUUGGUACACAAGGUAAAC | 2019 |
| | sasa | | | | | |
| AD-1736003 | csuscuu(Ada)UfuUfAfAfcuuaaccg | 1482 | VPusAfscggUfuAfAfguuaAfaUfaagagsusu | 1751 | AACUCUUAUUUAACUUAACCGUC | 2020 |
| | susa | | | | | |
| AD-1736004 | usgscuu(Gda)AfuGfAfUfacaaugaa | 1483 | VPusAfsuucAfuUfGfuaucAfuCfaagcasusu | 1752 | AAUGCUUGAUGAUACAAUGAAUG | 2021 |
| | susa | | | | | |
| AD-1736005 | asascug(Gda)GfaAfAfGfacaauuug | 1484 | VPusUfscaaAfuUfGfucuuUfcCfcaguusasc | 1753 | GUAACUGGGAAAGACAAUUUGAA | 2022 |
| | sasa | | | | | |
| AD-1736006 | csusccu(Uda)AfuAfUfGfacuauuug | 1485 | VPusUfscaaAfuAfGfucauAfuAfaggagscsc | 1754 | GGCUCCUUAUAUGACUAUUUGAA | 2023 |
| | sasa | | | | | |
| AD-1736007 | asgsuca(Uda)GfcUfAfAfccaaugga | 1486 | VPusUfsuccAfuUfGfguuaGfcAfugacusgsa | 1755 | UCAGUCAUGCUAACCAAUGGAAA | 2024 |
| | sasa | | | | | |
| AD-1736008 | cscsuga(Ada)GfuCfAfCfacagcuaas | 1487 | VPusAfsuuaGfcUfGfugugAfcUfucaggsasc | 1756 | GUCCUGAAGUCACACAGCUAAUG | 2025 |
| | usa | | | | | |
| AD-1736009 | ususgcu(Gda)CfuGfAfCfaacaaaga | 1488 | VPusAfsucuUfuGfUfugucAfgCfagcaasusg | 1757 | CAUUGCUGCUGACAACAAAGAUA | 2026 |
| | susa | | | | | |
| AD-1736010 | usascug(Ada)CfuUfUfGfccuguaaa | 1489 | VPusCfsuuuAfcAfGfgcaaAfgUfcaguasasu | 1758 | AUUACUGACUUUGCCUGUAAAGA | 2027 |
| | sgsa | | | | | |
| AD-1736011 | asasucc(Cda)UfaUfUfUfuaaaauucs | 1490 | VPusGfsgaaUfuUfUfaaaaUfaGfggauusgsu | 1759 | ACAAUCCCUAUUUUAAAAUUCCC | 2028 |
| | csa | | | | | |
| AD-1736012 | gsusgcc(Uda)UfaGfGfAfgauuaccc | 1491 | VPusAfsgggUfaAfUfcuccUfaAfggcacsusc | 1760 | GAGUGCCUUAGGAGAUUACCCUU | 2029 |
| | susa | | | | | |
| AD-1736013 | gsascaa(Uda)CfuAfAfAfuauaucau | 1492 | VPusGfsaugAfuAfUfauuuAfgAfuugucsusg | 1761 | CAGACAAUCUAAAUAUAUCAUCA | 2030 |
| | scsa | | | | | |
| AD-1736014 | usgsuga(Uda)AfaUfGfAfacaccgua | 1493 | VPusUfsuacGfgUfGfuucaUfuAfucacasasg | 1762 | CUUGUGAUAAUGAACACCGUAAG | 2031 |
| | sasa | | | | | |
| AD-1736015 | uscsacc(Gda)UfuGfUfAfaagacuuu | 1494 | VPusAfsaaaGfuCfUfuuacAfaCfggugasusu | 1763 | AAUCACCGUUGUAAAGACUUUUU | 2032 |
| | susa | | | | | |
| AD-1736016 | ususaga(Gda)AfaGfCfUfuccugaca | 1495 | VPusAfsuguCfaGfGfaagcUfuCfucuaasusc | 1764 | GAUUAGAGAAGCUUCCUGACAUC | 2033 |
| | susa | | | | | |
| AD-1736017 | asascag(Uda)AfaAfGfAfaaugcuac | 1496 | VPusAfsguaGfcAfUfuucuUfuAfcuguusasu | 1765 | AUAACAGUAAAGAAAUGCUACUU | 2034 |
| | susa | | | | | |
| AD-1736018 | usasugu(Uda)CfuGfAfCfuugagagu | 1497 | VPusAfsacuCfuCfAfagucAfgAfacauasasa | 1766 | UUUAUGUUCUGACUUGAGAGUUA | 2035 |
| | susa | | | | | |
| AD-1736019 | ususaga(Gda)CfaUfGfCfuaucuuua | 1498 | VPusCfsuaaAfgAfUfagcaUfgCfucuaasusu | 1767 | AAUUAGAGCAUGCUAUCUUUAGG | 2036 |
| | sgsa | | | | | |
| AD-1736020 | usasguc(Uda)UfuGfAfUfugaaauaa | 1499 | VPusCfsuuaUfuUfCfaaucAfaAfgacuasusg | 1768 | CAUAGUCUUUGAUUGAAAUAAGU | 2037 |
| | sgsa | | | | | |
| AD-1736021 | ususggu(Ada)AfaCfGfAfgauuuaac | 1500 | VPusUfsguuAfaAfUfcucgUfuUfaccaasasg | 1769 | CUUUGGUAAACGAGAUUUAACAU | 2038 |
| | sasa | | | | | |
| AD-1736022 | gsuscug(Ada)AfaAfUfUfgcuuucau | 1501 | VPusAfsaugAfaAfGfcaauUfuUfcagacscsu | 1770 | AGGUCUGAAAAUUGCUUUCAUUU | 2039 |
| | susa | | | | | |
| AD-1736023 | asusgaa(Cda)UfuGfUfUfgccuugua | 1502 | VPusUfsuacAfaGfGfcaacAfaGfuucausgsc | 1771 | GCAUGAACUUGUUGCCUUGUAAA | 2040 |
| | sasa | | | | | |
| AD-1736024 | gsgsgca(Ada)UfaAfAfCfuguaucaa | 1503 | VPusUfsuugAfuAfCfaguuUfaUfugcccsasa | 1772 | UUGGGCAAUAAACUGUAUCAAAA | 2041 |
| | sasa | | | | | |
| AD-1736025 | ascsaug(Uda)UfaGfCfAfuauaaugu | 1504 | VPusUfsacaUfuAfUfaugcUfaAfcaugusasc | 1773 | GUACAUGUUAGCAUAUAAUGUAU | 2042 |
| | sasa | | | | | |
| AD-1736026 | usgsuga(Gda)AfgUfAfUfagaauuuc | 1505 | VPusAfsgaaAfuUfCfuauaCfuCfucacasgsu | 1774 | ACUGUGAGAGUAUAGAAUUUCUU | 2043 |
| | susa | | | | | |
| AD-1736027 | asusccu(Uda)CfaUfUfUfggcacuau | 1506 | VPusUfsauaGfuGfCfcaaaUfgAfaggaususa | 1775 | UAAUCCUUCAUUUGGCACUAUAG | 2044 |
| | sasa | | | | | |
| AD-1736028 | ascsauu(Uda)AfgAfAfAfguagcuuu | 1507 | VPusUfsaaaGfcUfAfcuuuCfuAfaaugusgsu | 1776 | ACACAUUUAGAAAGUAGCUUUAU | 2045 |
| | sasa | | | | | |
| AD-1736029 | asascug(Uda)UfcUfAfUfaaagcaaas | 1508 | VPusCfsuuuGfcUfUfuauaGfaAfcaguusasa | 1777 | UUAACUGUUCUAUAAAGCAAAGC | 2046 |
| | gsa | | | | | |
| AD-1736030 | cscsucc(Ada)GfaGfAfUfgaaagauc | 1509 | VPusAfsgauCfuUfUfcaucUfcUfggaggsasg | 1778 | CUCCUCCAGAGAUGAAAGAUCUU | 2047 |
| | susa | | | | | |
| AD-1736031 | ususguu(Uda)AfaCfUfAfugacuccu | 1510 | VPusUfsaggAfgUfCfauagUfuAfaacaascsa | 1779 | UGUUGUUUAACUAUGACUCCUAA | 2048 |
| | sasa | | | | | |
| AD-1736032 | csusguu(Uda)GfaCfAfAfugcuuugu | 1511 | VPusAfsacaAfaGfCfauugUfcAfaacagsasa | 1780 | UUCUGUUUGACAAUGCUUUGUUC | 2049 |
| | susa | | | | | |
| AD-1736033 | usgsucc(Uda)AfaAfAfGfaaauuuuu | 1512 | VPusGfsaaaAfaUfUfucuuUfuAfggacasasc | 1781 | GUUGUCCUAAAAGAAAUUUUUCU | 2050 |
| | scsa | | | | | |
| AD-1736034 | ascsaga(Uda)UfgAfAfCfaaagaacus | 1513 | VPusAfsaguUfcUfUfuguuCfaAfucugusasg | 1782 | CUACAGAUUGAACAAAGAACUUA | 2051 |
| | usa | | | | | |
| AD-1736035 | gsusuua(Ada)CfcUfGfUfccaaacuu | 1514 | VPusGfsaagUfuUfGfgacaGfgUfuaaacsgsa | 1783 | UCGUUUAACCUGUCCAAACUUCU | 2052 |
| | scsa | | | | | |
| AD-1736036 | csasaaa(Uda)GfuUfUfAfacuuuacc | 1515 | VPusUfsgguAfaAfGfuuaaAfcAfuuuugsgsu | 1784 | ACCAAAAUGUUUAACUUUACCAA | 2053 |
| | sasa | | | | | |
| AD-1736037 | uscscca(Uda)UfgUfGfUfaauauuua | 1516 | VPusAfsuaaAfuAfUfuacaCfaAfugggascsu | 1785 | AGUCCCAUUGUGUAAUAUUUAUU | 2054 |
| | susa | | | | | |
| AD-1736038 | gsusugg(Gda)UfaAfAfUfaugcuuau | 1517 | VPusAfsauaAfgCfAfuauuUfaCfccaacsasu | 1786 | AUGUUGGGUAAAUAUGCUUAUUG | 2055 |
| | susa | | | | | |
| AD-1736039 | csasggg(Uda)UfgUfCfUfuugagucu | 1518 | VPusCfsagaCfuCfAfaagaCfaAfcccugsasg | 1787 | CUCAGGGUUGUCUUUGAGUCUGC | 2056 |
| | sgsa | | | | | |
| AD-1736040 | gscsuaa(Ada)AfuCfCfAfacugcaau | 1519 | VPusAfsauuGfcAfGfuuggAfuUfuuagcscsc | 1788 | GGGCUAAAAUCCAACUGCAAUUG | 2057 |
| | susa | | | | | |
| AD-1736041 | csasgga(Cda)UfgAfAfGfuguguaug | 1520 | VPusAfscauAfcAfCfacuuCfaGfuccugsgsc | 1789 | GCCAGGACUGAAGUGUGUAUGUC | 2058 |
| | susa | | | | | |
| AD-1736042 | gsasgua(Cda)CfaAfUfUfgccuuauu | 1521 | VPusUfsaauAfaGfGfcaauUfgGfuacucscsu | 1790 | AGGAGUACCAAUUGCCUUAUUAU | 2059 |
| | sasa | | | | | |
| AD-1736043 | asasgug(Cda)CfuAfUfGfugaaguga | 1522 | VPusAfsucaCfuUfCfacauAfgGfcacuususu | 1791 | AAAAGUGCCUAUGUGAAGUGAUU | 2060 |
| | susa | | | | | |
| AD-1736044 | usasaca(Ada)UfaAfUfAfgaacugcc | 1523 | VPusUfsggcAfgUfUfcuauUfaUfuguuasgsg | 1792 | CCUAACAAUAAUAGAACUGCCAG | 2061 |
| | sasa | | | | | |
| AD-1736045 | gscsugu(Uda)CfcAfUfAfccauugcu | 1524 | VPusAfsagcAfaUfGfguauGfgAfacagcsasg | 1793 | CUGCUGUUCCAUACCAUUGCUUU | 2062 |
| | susa | | | | | |
| AD-1736046 | usasgac(Uda)GfgAfGfAfagauuauu | 1525 | VPusGfsaauAfaUfCfuucuCfcAfgucuasgsa | 1794 | UCUAGACUGGAGAAGAUUAUUCA | 2063 |
| | scsa | | | | | |
| AD-1736047 | ususcca(Ada)CfaGfCfAfucaccaaas | 1526 | VPusAfsuuuGfgUfGfaugcUfgUfuggaasgsg | 1795 | CCUUCCAACAGCAUCACCAAAUG | 2064 |
| | usa | | | | | |
| AD-1736048 | usasauu(Gda)GfuAfCfAfgauucugu | 1527 | VPusAfsacaGfaAfUfcuguAfcCfaauuascsa | 1796 | UGUAAUUGGUACAGAUUCUGUUG | 2065 |
| | susa | | | | | |
| AD-1736049 | gsasgaa(Ada)CfaUfUfAfuuuguugu | 1528 | VPusGfsacaAfcAfAfauaaUfgUfuucucsusu | 1797 | AAGAGAAACAUUAUUUGUUGUCA | 2066 |
| | scsa | | | | | |
| AD-1736050 | csgscuc(Uda)CfaAfUfUfgcuagugg | 1529 | VPusAfsccaCfuAfGfcaauUfgAfgagcgscsc | 1798 | GGCGCUCUCAAUUGCUAGUGGUC | 2067 |
| | susa | | | | | |
| AD-1736051 | csgsgua(Uda)CfaGfAfAfacagcaaas | 1530 | VPusAfsuuuGfcUfGfuuucUfgAfuaccgsusc | 1799 | GACGGUAUCAGAAACAGCAAAUA | 2068 |
| | usa | | | | | |
| AD-1736052 | cscscag(Ada)CfaUfAfAfuuuuauau | 1531 | VPusAfsauaUfaAfAfauuaUfgUfcugggsasg | 1800 | CUCCCAGACAUAAUUUUAUAUUU | 2069 |
| | susa | | | | | |
| AD-1736053 | usasgua(Cda)AfuUfUfUfgagguauu | 1532 | VPusAfsaauAfcCfUfcaaaAfuGfuacuasasc | 1801 | GUUAGUACAUUUUGAGGUAUUUU | 2070 |
| | susa | | | | | |
| AD-1736054 | asasaug(Cda)UfaUfUfGfauaacagu | 1533 | VPusUfsacuGfuUfAfucaaUfaGfcauuusasc | 1802 | GUAAAUGCUAUUGAUAACAGUAA | 2071 |
| | sasa | | | | | |
| AD-1736055 | usasgcu(Gda)AfaUfUfUfgggacuau | 1534 | VPusCfsauaGfuCfCfcaaaUfuCfagcuasusc | 1803 | GAUAGCUGAAUUUGGGACUAUGU | 2072 |
| | sgsa | | | | | |
| AD-1736056 | asusugu(Gda)GfuUfUfUfcaaagaua | 1535 | VPusAfsuauCfuUfUfgaaaAfcCfacaausgsg | 1804 | CCAUUGUGGUUUUCAAAGAUAUU | 2073 |
| | susa | | | | | |
| AD-1736057 | cscsugg(Uda)UfgCfAfGfuaucugaa | 1536 | VPusUfsuucAfgAfUfacugCfaAfccaggsusu | 1805 | AACCUGGUUGCAGUAUCUGAAAA | 2074 |
| | sasa | | | | | |
| AD-1736058 | usasgaa(Uda)GfgUfUfGfuugagcua | 1537 | VPusUfsuagCfuCfAfacaaCfcAfuucuascsa | 1806 | UGUAGAAUGGUUGUUGAGCUAAG | 2075 |
| | sasa | | | | | |
| AD-1736059 | csusggc(Cda)AfuCfAfUfuauuacug | 1538 | VPusAfscagUfaAfUfaaugAfuGfgccagscsu | 1807 | AGCUGGCCAUCAUUAUUACUGUG | 2076 |
| | susa | | | | | |
| AD-1736060 | ususgac(Ada)GfaUfAfAfaauacgaa | 1539 | VPusCfsuucGfuAfUfuuuaUfcUfgucaasusu | 1808 | AAUUGACAGAUAAAAUACGAAGU | 2077 |
| | sgsa | | | | | |
| AD-1736061 | uscsaca(Ada)AfcAfCfAfucauuacas | 1540 | VPusUfsuguAfaUfGfauguGfuUfugugasasu | 1809 | AUUCACAAACACAUCAUUACAAG | 2078 |
| | asa | | | | | |
| AD-1736062 | cscsaug(Ada)UfuGfUfAfugaaaaua | 1541 | VPusCfsuauUfuUfCfauacAfaUfcauggsasa | 1810 | UUCCAUGAUUGUAUGAAAAUAGU | 2079 |
| | sgsa | | | | | |
| AD-1736063 | csascag(Ada)GfuAfAfUfgaauauuu | 1542 | VPusUfsaaaUfaUfUfcauuAfcUfcugugsusc | 1811 | GACACAGAGUAAUGAAUAUUUAA | 2080 |
| | sasa | | | | | |
| AD-1736064 | asgscug(Cda)UfuAfGfUfggaagaug | 1543 | VPusAfscauCfuUfCfcacuAfaGfcagcuscsc | 1812 | GGAGCUGCUUAGUGGAAGAUGUA | 2081 |
| | susa | | | | | |
| AD-1736065 | usasgcu(Cda)UfgUfGfUfgcugauac | 1544 | VPusGfsguaUfcAfGfcacaCfaGfagcuasgsa | 1813 | UCUAGCUCUGUGUGCUGAUACCU | 2082 |
| | scsa | | | | | |
| AD-1736066 | csasuuc(Gda)AfgAfGfAfauaugagc | 1545 | VPusAfsgcuCfaUfAfuucuCfuCfgaaugsusa | 1814 | UACAUUCGAGAGAAUAUGAGCUG | 2083 |
| | susa | | | | | |
| AD-1736067 | asusgca(Uda)UfuUfCfUfgaaaaugu | 1546 | VPusUfsacaUfuUfUfcagaAfaAfugcausasc | 1815 | GUAUGCAUUUUCUGAAAAUGUAU | 2084 |
| | sasa | | | | | |
| AD-1736068 | gsusuuc(Gda)AfcCfAfAfguauccca | 1547 | VPusUfsuggGfaUfAfcuugGfuCfgaaacsusu | 1816 | AAGUUUCGACCAAGUAUCCCAAA | 2085 |
| | sasa | | | | | |
| AD-1736069 | uscsuug(Cda)UfuGfCfCfacaauauc | 1548 | VPusCfsgauAfuUfGfuggcAfaGfcaagasusa | 1817 | UAUCUUGCUUGCCACAAUAUCGG | 2086 |
| | sgsa | | | | | |
| AD-1736070 | gsgsgua(Cda)AfaGfAfGfggaauaca | 1549 | VPusUfsuguAfuUfCfccucUfuGfuacccsgsa | 1818 | UCGGGUACAAGAGGGAAUACAAA | 2087 |
| | sasa | | | | | |
| AD-1736071 | gsgsuga(Uda)CfaAfAfUfccuguguc | 1550 | VPusAfsgacAfcAfGfgauuUfgAfucaccsusg | 1819 | CAGGUGAUCAAAUCCUGUGUCUC | 2088 |
| | susa | | | | | |
| AD-1736072 | csasgug(Gda)GfuUfUfCfuaggauag | 1551 | VPusCfscuaUfcCfUfagaaAfcCfcacugsusc | 1820 | GACAGUGGGUUUCUAGGAUAGGU | 2089 |
| | sgsa | | | | | |
| AD-1736073 | asascua(Ada)GfuAfUfUfuuuaagga | 1552 | VPusGfsuccUfuAfAfaaauAfcUfuaguusasc | 1821 | GUAACUAAGUAUUUUUAAGGACA | 2090 |
| | scsa | | | | | |
| AD-1736074 | csasuaa(Uda)GfuGfAfUfuguuaaau | 1553 | VPusAfsauuUfaAfCfaaucAfcAfuuaugsgsg | 1822 | CCCAUAAUGUGAUUGUUAAAUUU | 2091 |
| | susa | | | | | |
| AD-1736075 | gsasgaa(Gda)AfaUfUfAfagaaaacu | 1554 | VPusGfsaguUfuUfCfuuaaUfuCfuucucsasa | 1823 | UUGAGAAGAAUUAAGAAAACUCU | 2092 |
| | scsa | | | | | |
| AD-1736076 | gsasaga(Gda)AfaAfUfUfgauuguuu | 1555 | VPusUfsaaaCfaAfUfcaauUfuCfucuucsusc | 1824 | GAGAAGAGAAAUUGAUUGUUUAU | 2093 |
| | sasa | | | | | |
| AD-1736077 | asgsugu(Gda)AfaAfCfUfugugccau | 1556 | VPusUfsaugGfcAfCfaaguUfuCfacacususu | 1825 | AAAGUGUGAAACUUGUGCCAUAG | 2094 |
| | sasa | | | | | |
| AD-1736078 | csusgag(Ada)CfaCfUfGfagaaaugu | 1557 | VPusGfsacaUfuUfCfucagUfgUfcucagsgsa | 1826 | UCCUGAGACACUGAGAAAUGUCC | 2095 |
| | scsa | | | | | |
| AD-1736079 | usgsgcu(Uda)AfuAfUfGfuguuuaaa | 1558 | VPusCfsuuuAfaAfCfacauAfuAfagccasasu | 1827 | AUUGGCUUAUAUGUGUUUAAAGA | 2096 |
| | sgsa | | | | | |
| AD-1736080 | usgscau(Uda)UfuGfAfCfauugcaaa | 1559 | VPusUfsuuuGfcAfAfugucAfaAfaugcasasu | 1828 | AUUGCAUUUUGACAUUGCAAAAC | 2097 |
| | sasa | | | | | |
| AD-1736081 | ususagg(Uda)UfaGfAfAfguuccaga | 1560 | VPusUfsucuGfgAfAfcuucUfaAfccuaasusg | 1829 | CAUUAGGUUAGAAGUUCCAGAAU | 2098 |
| | sasa | | | | | |
| AD-1736082 | usgscga(Cda)AfuGfAfAfaacauccu | 1561 | VPusAfsaggAfuGfUfuuucAfuGfucgcasgsc | 1830 | GCUGCGACAUGAAAACAUCCUUG | 2099 |
| | susa | | | | | |
| AD-1736083 | gsasgag(Gda)AfuUfUfUfuuaccauc | 1562 | VPusAfsgauGfgUfAfaaaaAfuCfcucucsusu | 1831 | AAGAGAGGAUUUUUUACCAUCUC | 2100 |
| | susa | | | | | |
| AD-1736084 | gsasagg(Uda)GfaAfGfAfgaauuuca | 1563 | VPusUfsugaAfaUfUfcucuUfcAfccuucscsa | 1832 | UGGAAGGUGAAGAGAAUUUCAAA | 2101 |
| | sasa | | | | | |
| AD-1736085 | usgsgua(Uda)CfuGfAfAfuaucauga | 1564 | VPusUfsucaUfgAfUfauucAfgAfuaccasgsc | 1833 | GCUGGUAUCUGAAUAUCAUGAAC | 2102 |
| | sasa | | | | | |
| AD-1736086 | usgsuug(Gda)UfuUfAfAfuuuuuca | 1565 | VPusGfsuugAfaAfAfauuaAfaCfcaacasusg | 1834 | CAUGUUGGUUUAAUUUUUCAACC | 2103 |
| | ascsa | | | | | |
| AD-1736087 | usasguu(Gda)UfaAfUfUfuaaaugug | 1566 | VPusCfscacAfuUfUfaaauUfaCfaacuascsu | 1835 | AGUAGUUGUAAUUUAAAUGUGGA | 2104 |
| | sgsa | | | | | |
| AD-1736088 | gsusgaa(Uda)GfuUfUfUfugccauuu | 1567 | VPusAfsaaaUfgGfCfaaaaAfcAfuucacsasu | 1836 | AUGUGAAUGUUUUUGCCAUUUUU | 2105 |
| | susa | | | | | |
| AD-1736089 | csasgag(Ada)UfgAfUfUfuuucuuuu | 1568 | VPusUfsaaaAfgAfAfaaauCfaUfcucugscsc | 1837 | GGCAGAGAUGAUUUUUCUUUUAA | 2106 |
| | sasa | | | | | |
| AD-1736090 | asgsaaa(Ada)UfuAfGfAfuucagauc | 1569 | VPusAfsgauCfuGfAfaucuAfaUfuuucusgsu | 1838 | ACAGAAAAUUAGAUUCAGAUCUC | 2107 |
| | susa | | | | | |
| AD-1736091 | ascsugg(Ada)AfaAfUfUfaagaaagu | 1570 | VPusUfsacuUfuCfUfuaauUfuUfccagusgsg | 1839 | CCACUGGAAAAUUAAGAAAGUAG | 2108 |
| | sasa | | | | | |
| AD-1736092 | usasaag(Uda)GfgGfAfAfagauaaua | 1571 | VPusUfsuauUfaUfCfuuucCfcAfcuuuascsa | 1840 | UGUAAAGUGGGAAAGAUAAUAAA | 2109 |
| | sasa | | | | | |
| AD-1736093 | csusagg(Cda)UfaAfGfAfaagaguug | 1572 | VPusAfscaaCfuCfUfuucuUfaGfccuagsusu | 1841 | AACUAGGCUAAGAAAGAGUUGUA | 2110 |
| | susa | | | | | |
| AD-1736094 | gscsuga(Uda)AfaAfAfUfuaauggau | 1573 | VPusUfsaucCfaUfUfaauuUfuAfucagcsasc | 1842 | GUGCUGAUAAAAUUAAUGGAUAA | 2111 |
| | sasa | | | | | |
| AD-1736095 | cscsugu(Ada)UfuUfCfUfuuaguugu | 1574 | VPusGfsacaAfcUfAfaagaAfaUfacaggsasa | 1843 | UUCCUGUAUUUCUUUAGUUGUCG | 2112 |
| | scsa | | | | | |
| AD-1736096 | ususugu(Uda)UfuGfCfUfuuugacaa | 1575 | VPusUfsuugUfcAfAfaagcAfaAfacaaasgsc | 1844 | GCUUUGUUUUGCUUUUGACAAAC | 2113 |
| | sasa | | | | | |
| AD-1736097 | usgsgcu(Gda)UfgAfAfAfauauucuc | 1576 | VPusGfsgagAfaUfAfuuuuCfaCfagccascsa | 1845 | UGUGGCUGUGAAAAUAUUCUCCU | 2114 |
| | scsa | | | | | |
| AD-1736098 | ascsuau(Uda)UfuAfGfGfcauagcac | 1577 | VPusAfsgugCfuAfUfgccuAfaAfauagusgsc | 1846 | GCACUAUUUUAGGCAUAGCACUU | 2115 |
| | susa | | | | | |
| AD-1736099 | ususuca(Gda)UfaCfUfGfuauaaagu | 1578 | VPusCfsacuUfuAfUfacagUfaCfugaaasasu | 1847 | AUUUUCAGUACUGUAUAAAGUGG | 2116 |
| | sgsa | | | | | |
| AD-1736100 | gsusgau(Gda)CfaAfUfCfuauguuuc | 1579 | VPusGfsgaaAfcAfUfagauUfgCfaucacscsu | 1848 | AGGUGAUGCAAUCUAUGUUUCCC | 2117 |
| | scsa | | | | | |
| AD-1736101 | usgsuuu(Gda)GfcAfGfAfucucauca | 1580 | VPusUfsugaUfgAfGfaucuGfcCfaaacasusu | 1849 | AAUGUUUGGCAGAUCUCAUCAAU | 2118 |
| | sasa | | | | | |
| AD-1736102 | asasggu(Uda)GfuUfUfGfugaccaga | 1581 | VPusUfsucuGfgUfCfacaaAfcAfaccuususc | 1850 | GAAAGGUUGUUUGUGACCAGAAG | 2119 |
| | sasa | | | | | |
| AD-1736103 | gsgsaag(Gda)AfaAfUfAfuuuugaga | 1582 | VPusUfsucuCfaAfAfauauUfuCfcuuccsusu | 1851 | AAGGAAGGAAAUAUUUUGAGAAU | 2120 |
| | sasa | | | | | |
| AD-1736104 | ususuug(Gda)UfaGfCfGfugacucuu | 1583 | VPusGfsaagAfgUfCfacgcUfaCfcaaaasgsa | 1852 | UCUUUUGGUAGCGUGACUCUUCU | 2121 |
| | scsa | | | | | |
| AD-1736105 | usgsaac(Ada)GfgAfAfAfagauguaa | 1584 | VPusUfsuuaCfaUfCfuuuuCfcUfguucasusu | 1853 | AAUGAACAGGAAAAGAUGUAAAA | 2122 |
| | sasa | | | | | |
| AD-1736106 | gsgsgaa(Cda)CfaAfGfAfgguauaug | 1585 | VPusCfscauAfuAfCfcucuUfgGfuucccsasc | 1854 | GUGGGAACCAAGAGGUAUAUGGC | 2123 |
| | sgsa | | | | | |
| AD-1736107 | usgsgca(Cda)UfaGfGfUfccaaaucu | 1586 | VPusAfsagaUfuUfGfgaccUfaGfugccasgsu | 1855 | ACUGGCACUAGGUCCAAAUCUUG | 2124 |
| | susa | | | | | |
| AD-1736108 | csascac(Cda)UfuCfAfUfauggagau | 1587 | VPusAfsaucUfcCfAfuaugAfaGfgugugscsc | 1856 | GGCACACCUUCAUAUGGAGAUUG | 2125 |
| | susa | | | | | |
| AD-1736109 | csusggu(Uda)UfaCfUfGfggaaauag | 1588 | VPusGfscuaUfuUfCfccagUfaAfaccagsasc | 1857 | GUCUGGUUUACUGGGAAAUAGCC | 2126 |
| | scsa | | | | | |
| AD-1736110 | gscscca(Ada)AfgGfAfAfuguauaua | 1589 | VPusUfsuauAfuAfCfauucCfuUfugggcscsu | 1858 | AGGCCCAAAGGAAUGUAUAUAAG | 2127 |
| | sasa | | | | | |
| AD-1736111 | ususaac(Uda)GfcAfAfGfaguuuacu | 1590 | VPusCfsaguAfaAfCfucuuGfcAfguuaasasa | 1859 | UUUUAACUGCAAGAGUUUACUGU | 2128 |
| | sgsa | | | | | |
| AD-1736112 | gsasacc(Ada)CfuCfUfCfugagugca | 1591 | VPusUfsugcAfcUfCfagagAfgUfgguucscsu | 1860 | AGGAACCACUCUCUGAGUGCAAU | 2129 |
| | sasa | | | | | |
| AD-1736113 | ususauc(Uda)UfuUfCfAfuccuggca | 1592 | VPusAfsugcCfaGfGfaugaAfaAfgauaasasg | 1861 | CUUUAUCUUUUCAUCCUGGCAUU | 2130 |
| | susa | | | | | |
| AD-1736114 | gsusgag(Uda)GfuUfGfGfuaugccaa | 1593 | VPusGfsuugGfcAfUfaccaAfcAfcucacsgsc | 1862 | GCGUGAGUGUUGGUAUGCCAACG | 2131 |
| | scsa | | | | | |
| AD-1736115 | asgsaau(Gda)UfaUfUfUfggcuaaau | 1594 | VPusUfsauuUfaGfCfcaaaUfaCfauucuscsa | 1863 | UGAGAAUGUAUUUGGCUAAAUAA | 2132 |
| | sasa | | | | | |
| AD-1736116 | gsasacc(Cda)UfuUfUfAfuuaagagg | 1595 | VPusUfsccuCfuUfAfauaaAfaGfgguucscsa | 1864 | UGGAACCCUUUUAUUAAGAGGAG | 2133 |
| | sasa | | | | | |
| AD-1736117 | csusccu(Gda)UfcCfAfUfagcugcga | 1596 | VPusAfsucgCfaGfCfuaugGfaCfaggagsgsc | 1865 | GCCUCCUGUCCAUAGCUGCGAUG | 2134 |
| | susa | | | | | |
| AD-1736118 | ascsuga(Gda)UfuGfAfAfauaaggaa | 1597 | VPusCfsuucCfuUfAfuuucAfaCfucagusasa | 1866 | UUACUGAGUUGAAAUAAGGAAGG | 2135 |
| | sgsa | | | | | |
| AD-1736119 | csusgcc(Ada)AfaCfAfGfaaggagca | 1598 | VPusAfsugcUfcCfUfucugUfuUfggcagsgsu | 1867 | ACCUGCCAAACAGAAGGAGCAUG | 2136 |
| | susa | | | | | |
| AD-1736120 | gsgscaa(Ada)GfuUfGfUfgaagcacu | 1599 | VPusGfsaguGfcUfUfcacaAfcUfuugccsasc | 1868 | GUGGCAAAGUUGUGAAGCACUCC | 2137 |
| | scsa | | | | | |
| AD-1736121 | asusagg(Ada)UfgAfUfGfaaguuuag | 1600 | VPusUfscuaAfaCfUfucauCfaUfccuaususu | 1869 | AAAUAGGAUGAUGAAGUUUAGAG | 2138 |
| | sasa | | | | | |
| AD-1736122 | asusucg(Uda)AfuCfUfUfaaaauggc | 1601 | VPusUfsgccAfuUfUfuaagAfuAfcgaausasc | 1870 | GUAUUCGUAUCUUAAAAUGGCAC | 2139 |
| | sasa | | | | | |
| AD-1736123 | csascag(Cda)UfuGfGfGfuaauagcg | 1602 | VPusAfscgcUfaUfUfacccAfaGfcugugscsu | 1871 | AGCACAGCUUGGGUAAUAGCGUU | 2140 |
| | susa | | | | | |
| AD-1736124 | ususugc(Ada)AfcAfAfCfauaacacu | 1603 | VPusCfsaguGfuUfAfuguuGfuUfgcaaasasa | 1872 | UUUUUGCAACAACAUAACACUGC | 2141 |
| | sgsa | | | | | |
| AD-1736125 | usgscca(Cda)CfaUfGfUfgacuuauu | 1604 | VPusCfsaauAfaGfUfcacaUfgGfuggcasasa | 1873 | UUUGCCACCAUGUGACUUAUUGG | 2142 |
| | sgsa | | | | | |
| AD-1736126 | uscsgau(Ada)GfaGfGfAfaaugagaa | 1605 | VPusUfsuucUfcAfUfuuccUfcUfaucgasgsg | 1874 | CCUCGAUAGAGGAAAUGAGAAAG | 2143 |
| | sasa | | | | | |
| AD-1736127 | ususuuu(Ada)AfgUfUfAfgcaggacu | 1606 | VPusAfsaguCfcUfGfcuaaCfuUfaaaaasgsg | 1875 | CCUUUUUAAGUUAGCAGGACUUU | 2144 |
| | susa | | | | | |
| AD-1736128 | usgsuaa(Ada)UfaAfUfUfaucugccu | 1607 | VPusUfsaggCfaGfAfuaauUfaUfuuacasusc | 1876 | GAUGUAAAUAAUUAUCUGCCUAA | 2145 |
| | sasa | | | | | |
| AD-1736129 | usgsguu(Gda)UfaCfGfUfgccucaaa | 1608 | VPusAfsuuuGfaGfGfcacgUfaCfaaccasusa | 1877 | UAUGGUUGUACGUGCCUCAAAUA | 2146 |
| | susa | | | | | |

TABLE 6
Unmodified Sense and Antisense Strand Sequences of ACVR1C dsRNA Agents Comprising a GalNAc
Derivative Targeting Ligand

| | | SEQ | Range in | | SEQ | Range in |
|---|---|---|---|---|---|---|
| Duplex | Sense Sequence | ID | NM_ | Antisense Sequence | ID | NM_ |
| Name | 5′ to 3′ | NO: | 145259.3 | 5′ to 3′ | NO: | 145259.3 |
| AD-1736131 | UUUUUUCAAACUCCAUUCCUA | 802 | 3085-3105 | UAGGAAUGGAGUUUGAAAAAAGA | 1071 | 3083-3105 |
| AD-1736132 | ACUUUCAUCUGUCUUCAGAUA | 803 | 8677-8697 | UAUCUGAAGACAGAUGAAAGUUA | 1072 | 8675-8697 |
| AD-1736133 | CCUCAUAACUUCUCCAGUAAA | 804 | 6745-6765 | UUUACUGGAGAAGUUAUGAGGUG | 1073 | 6743-6765 |
| AD-1736134 | CUCACUAUCUUUCAACAUUAA | 805 | 5871-5891 | UUAAUGUUGAAAGAUAGUGAGGG | 1074 | 5869-5891 |
| AD-1736135 | CUCGUUAAUGCUCUCAUCCAA | 806 | 3562-3582 | UUGGAUGAGAGCAUUAACGAGUA | 1075 | 3560-3582 |
| AD-1736136 | GUUCUUCAUAAUCCACUACUA | 807 | 2292-2312 | UAGUAGUGGAUUAUGAAGAACAG | 1076 | 2290-2312 |
| AD-1736137 | GUUGACUUCAUCCAAUCUCUA | 808 | 1876-1896 | UAGAGAUUGGAUGAAGUCAACUC | 1077 | 1874-1896 |
| AD-1736138 | CUACACAAUGAACUUCUUAAA | 809 | 6692-6712 | UUUAAGAAGUUCAUUGUGUAGAA | 1078 | 6690-6712 |
| AD-1736139 | CACAUCUAGAAUUCUUAAUUA | 810 | 4835-4855 | UAAUUAAGAAUUCUAGAUGUGAC | 1079 | 4833-4855 |
| AD-1736140 | AAAUCUCUCAUAGCUUUCUUA | 811 | 1725-1745 | UAAGAAAGCUAUGAGAGAUUUCU | 1080 | 1723-1745 |
| AD-1736141 | CCUACUAUUGUAGAAUUACUA | 812 | 5093-5113 | UAGUAAUUCUACAAUAGUAGGUG | 1081 | 5091-5113 |
| AD-1736142 | AUCUUCUUUUAGUGCAUUAAA | 813 | 6589-6609 | UUUAAUGCACUAAAAGAAGAUGA | 1082 | 6587-6609 |
| AD-1736143 | CUCAUGUACUCUUCUGAUUCA | 814 | 7977-7997 | UGAAUCAGAAGAGUACAUGAGCU | 1083 | 7975-7997 |
| AD-1736144 | UUUCUCUUUGUACCUUGGAAA | 815 | 6449-6469 | UUUCCAAGGUACAAAGAGAAAGA | 1084 | 6447-6469 |
| AD-1736145 | UGGCUUAUACAUCUUCAGAAA | 816 | 7515-7535 | UUUCUGAAGAUGUAUAAGCCAGA | 1085 | 7513-7535 |
| AD-1736146 | CAAAACAAAACUACUCCUAUA | 817 | 5120-5140 | UAUAGGAGUAGUUUUGUUUUGUU | 1086 | 5118-5140 |
| AD-1736147 | ACUAGCAGAACUCUUAUGAAA | 818 | 6055-6075 | UUUCAUAAGAGUUCUGCUAGUAA | 1087 | 6053-6075 |
| AD-1736148 | CCAAGUCACUCUAUAAUUCCA | 819 | 6635-6655 | UGGAAUUAUAGAGUGACUUGGGA | 1088 | 6633-6655 |
| AD-1736149 | UCCAUUUUUCUUGUCAUUAUA | 820 | 4114-4134 | UAUAAUGACAAGAAAAAUGGAGA | 1089 | 4112-4134 |
| AD-1736150 | CAACACCUCAACUCAUCUUUA | 821 | 1923-1943 | UAAAGAUGAGUUGAGGUGUUGCU | 1090 | 1921-1943 |
| AD-1736151 | ACUCUGAAAGAUCUGAUUUAA | 822 | 716-736 | UUAAAUCAGAUCUUUCAGAGUUU | 1091 | 714-736 |
| AD-1736152 | CUAGUUCUUUUCCGCAAUGAA | 823 | 7946-7966 | UUCAUUGCGGAAAAGAACUAGAA | 1092 | 7944-7966 |
| AD-1736153 | CUUUCACUCUGAAGAAAUCCA | 824 | 4189-4209 | UGGAUUUCUUCAGAGUGAAAGUU | 1093 | 4187-4209 |
| AD-1736154 | UCUAACUUUUCUCUCUUUCUA | 825 | 6525-6545 | UAGAAAGAGAGAAAAGUUAGAUA | 1094 | 6523-6545 |
| AD-1736155 | UCUCAACUUUGUGUCAAAGAA | 826 | 1667-1687 | UUCUUUGACACAAAGUUGAGAUA | 1095 | 1665-1687 |
| AD-1736156 | CUUCAAUAAUCAUUCCUUUAA | 827 | 5445-5465 | UUAAAGGAAUGAUUAUUGAAGAC | 1096 | 5443-5465 |
| AD-1736157 | UAACUGCUCUUCGUAUUAAGA | 828 | 1638-1658 | UCUUAAUACGAAGAGCAGUUAGG | 1097 | 1636-1658 |
| AD-1736158 | UCUUCUGUCAUAGUUCCAACA | 829 | 438-458 | UGUUGGAACUAUGACAGAAGACU | 1098 | 436-458 |
| AD-1736159 | AUCUCAGAACCAUAUCUGUUA | 830 | 2584-2604 | UAACAGAUAUGGUUCUGAGAUUU | 1099 | 2582-2604 |
| AD-1736160 | UGAAUCUAUCUUCAUUUUACA | 831 | 3483-3503 | UGUAAAAUGAAGAUAGAUUCAGU | 1100 | 3481-3503 |
| AD-1736161 | ACCACUAAACUUGUUCCUUUA | 832 | 2326-2346 | UAAAGGAACAAGUUUAGUGGUUC | 1101 | 2324-2346 |
| AD-1736162 | AGUGUUCUUUACUCCUUACAA | 833 | 4578-4598 | UUGUAAGGAGUAAAGAACACUGA | 1102 | 4576-4598 |
| AD-1736163 | CUUUACUCUUAACAGGAUUAA | 834 | 4367-4387 | UUAAUCCUGUUAAGAGUAAAGGA | 1103 | 4365-4387 |
| AD-1736164 | UAUACCUAAGAACAUAUUACA | 835 | 8153-8173 | UGUAAUAUGUUCUUAGGUAUAAG | 1104 | 8151-8173 |
| AD-1736165 | CUUCUACUGAGAUGAUCCAAA | 836 | 3114-3134 | UUUGGAUCAUCUCAGUAGAAGAA | 1105 | 3112-3134 |
| AD-1736166 | CUGUAUUUUCUCAUAGAGUAA | 837 | 3420-3440 | UUACUCUAUGAGAAAAUACAGAA | 1106 | 3418-3440 |
| AD-1736167 | ACUUUUCUCUUUCAGUUGUAA | 838 | 7116-7136 | UUACAACUGAAAGAGAAAAGUAU | 1107 | 7114-7136 |
| AD-1736168 | AUUAAUUUCUAGUCUGUGAAA | 839 | 8493-8513 | UUUCACAGACUAGAAAUUAAUGA | 1108 | 8491-8513 |
| AD-1736169 | CUCUGUAUAAGAGGUUUCACA | 840 | 6726-6746 | UGUGAAACCUCUUAUACAGAGAA | 1109 | 6724-6746 |
| AD-1736170 | GACUUUCAAAGUACUAAUACA | 841 | 8098-8118 | UGUAUUAGUACUUUGAAAGUCAA | 1110 | 8096-8118 |
| AD-1736171 | UUUGCCAUUAUACAAAGUUUA | 842 | 5262-5282 | UAAACUUUGUAUAAUGGCAAACA | 1111 | 5260-5282 |
| AD-1736172 | GAACACUAUCGACAUACCUCA | 843 | 1276-1296 | UGAGGUAUGUCGAUAGUGUUCAG | 1112 | 1274-1296 |
| AD-1736173 | UAGAAUGACAUUUACUAAUAA | 844 | 7818-7838 | UUAUUAGUAAAUGUCAUUCUACG | 1113 | 7816-7838 |
| AD-1736174 | AAGCUACUUAGAAAUGUUUAA | 845 | 5491-5511 | UUAAACAUUUCUAAGUAGCUUGU | 1114 | 5489-5511 |
| AD-1736175 | CCUUAGUACUCUCAGAGAUUA | 846 | 5968-5988 | UAAUCUCUGAGAGUACUAAGGGA | 1115 | 5966-5988 |
| AD-1736176 | UGAAUGCACACUAACGUAAUA | 847 | 6163-6183 | UAUUACGUUAGUGUGCAUUCAAG | 1116 | 6161-6183 |
| AD-1736177 | AAGCCUAAUGAUGAUAAUUAA | 848 | 1695-1715 | UUAAUUAUCAUCAUUAGGCUUUG | 1117 | 1693-1715 |
| AD-1736178 | AAACUUUUUACUAUCCCAUAA | 849 | 3341-3361 | UUAUGGGAUAGUAAAAAGUUUCC | 1118 | 3339-3361 |
| AD-1736179 | CUAGCUGAUACUCUUAAGUAA | 850 | 8268-8288 | UUACUUAAGAGUAUCAGCUAGCU | 1119 | 8266-8288 |
| AD-1736180 | AGCAUGAAAAGAUAACUCUAA | 851 | 1837-1857 | UUAGAGUUAUCUUUUCAUGCUGC | 1120 | 1835-1857 |
| AD-1736181 | UAAGCCAUUAUGCUAUUAGUA | 852 | 7017-7037 | UACUAAUAGCAUAAUGGCUUAGU | 1121 | 7015-7037 |
| AD-1736182 | UCUAGCUUUUAGCUAACAUAA | 853 | 6850-6870 | UUAUGUUAGCUAAAAGCUAGAAU | 1122 | 6848-6870 |
| AD-1736183 | GUGUGAUUCUUCAAACUUCAA | 854 | 316-336 | UUGAAGUUUGAAGAAUCACACAA | 1123 | 314-336 |
| AD-1736184 | UCUCCAGACCUAACAGUUUUA | 855 | 7247-7267 | UAAAACUGUUAGGUCUGGAGAGA | 1124 | 7245-7267 |
| AD-1736185 | CUUUUUUUCAUCUAGCCUUGA | 856 | 5808-5828 | UCAAGGCUAGAUGAAAAAAAGAA | 1125 | 5806-5828 |
| AD-1736186 | CAAAAUAAUUCUUGACAUCUA | 857 | 7552-7572 | UAGAUGUCAAGAAUUAUUUUGAC | 1126 | 7550-7572 |
| AD-1736187 | GCAUCACUUUCUGAAAAUAAA | 858 | 4690-4710 | UUUAUUUUCAGAAAGUGAUGCUG | 1127 | 4688-4710 |
| AD-1736188 | UCUUUUGUGAAACAUACUAUA | 859 | 5563-5583 | UAUAGUAUGUUUCACAAAAGAUG | 1128 | 5561-5583 |
| AD-1736189 | CUAUUUCUUCCAUAGGCUAAA | 860 | 7841-7861 | UUUAGCCUAUGGAAGAAAUAGAA | 1129 | 7839-7861 |
| AD-1736190 | CUUCAAUGAAGUGUUAACAAA | 861 | 2839-2859 | UUUGUUAACACUUCAUUGAAGAG | 1130 | 2837-2859 |
| AD-1736191 | UUUGCCUUCAUUCUACUUUCA | 862 | 2952-2972 | UGAAAGUAGAAUGAAGGCAAAAC | 1131 | 2950-2972 |
| AD-1736192 | AGGUUUUUAAAUGUCCUAAAA | 863 | 8035-8055 | UUUUAGGACAUUUAAAAACCUGA | 1132 | 8033-8055 |
| AD-1736193 | GAAUAUCUUUGAGUCCUUCAA | 864 | 1360-1380 | UUGAAGGACUCAAAGAUAUUCAC | 1133 | 1358-1380 |
| AD-1736194 | GAACACUUCCAAAGAUUAAUA | 865 | 2666-2686 | UAUUAAUCUUUGGAAGUGUUCAG | 1134 | 2664-2686 |
| AD-1736195 | CUAAAUUUUGUCUAACAAAUA | 866 | 7791-7811 | UAUUUGUUAGACAAAAUUUAGGC | 1135 | 7789-7811 |
| AD-1736196 | GUAAACAACUUAAAAUUGCUA | 867 | 5284-5304 | UAGCAAUUUUAAGUUGUUUACUC | 1136 | 5282-5304 |
| AD-1736197 | AAAAUAAUUUACUCACUCAGA | 868 | 6233-6253 | UCUGAGUGAGUAAAUUAUUUUAG | 1137 | 6231-6253 |
| AD-1736198 | GACAUCUAUUCUGUUGGUCUA | 869 | 1388-1408 | UAGACCAACAGAAUAGAUGUCAG | 1138 | 1386-1408 |
| AD-1736199 | UAACUGAGUAGUCUUAUAUUA | 870 | 4267-4287 | UAAUAUAAGACUACUCAGUUAGG | 1139 | 4265-4287 |
| AD-1736200 | UCUGGAAUUUAGCCUUCUAAA | 871 | 5936-5956 | UUUAGAAGGCUAAAUUCCAGACG | 1140 | 5934-5956 |
| AD-1736201 | ACAUGCCUUGAACUCUUGAAA | 872 | 8207-8227 | UUUCAAGAGUUCAAGGCAUGUUU | 1141 | 8205-8227 |
| AD-1736202 | AUCAAGCACUUCUUGCACUUA | 873 | 8782-8802 | UAAGUGCAAGAAGUGCUUGAUUU | 1142 | 8780-8802 |
| AD-1736203 | CUUGCUUUUCAUCUUUAUACA | 874 | 4915-4935 | UGUAUAAAGAUGAAAAGCAAGUG | 1143 | 4913-4935 |
| AD-1736204 | UUUUCUCCCUAUGUAAUAUCA | 875 | 2493-2513 | UGAUAUUACAUAGGGAGAAAAGC | 1144 | 2491-2513 |
| AD-1736205 | UGCUUUCUUCUAUGUCUAAAA | 876 | 4802-4822 | UUUUAGACAUAGAAGAAAGCAUA | 1145 | 4800-4822 |
| AD-1736206 | ACCUGCUUAGCAAAUCGUCUA | 877 | 5224-5244 | UAGACGAUUUGCUAAGCAGGUUC | 1146 | 5222-5244 |
| AD-1736207 | AUAUUCCUCUACCCUUUCACA | 878 | 2895-2915 | UGUGAAAGGGUAGAGGAAUAUCU | 1147 | 2893-2915 |
| AD-1736208 | UCCCACUUUUCUUUGGGUUUA | 879 | 6208-6228 | UAAACCCAAAGAAAAGUGGGAUU | 1148 | 6206-6228 |
| AD-1736209 | AGGCUUCUUGUAUAUACUUAA | 880 | 4442-4462 | UUAAGUAUAUACAAGAAGCCUGC | 1149 | 4440-4462 |
| AD-1736210 | AAACUUGAUCUGUGUUUCAGA | 881 | 8017-8037 | UCUGAAACACAGAUCAAGUUUGA | 1150 | 8015-8037 |
| AD-1736211 | UUUGCUUUAAUGCUAAAUUCA | 882 | 6885-6905 | UGAAUUUAGCAUUAAAGCAAAAG | 1151 | 6883-6905 |
| AD-1736212 | AGACUAGCUGUUAUUUGUAUA | 883 | 6830-6850 | UAUACAAAUAACAGCUAGUCUUU | 1152 | 6828-6850 |
| AD-1736213 | UUCCUACUUUCCCUUGAAUAA | 884 | 2469-2489 | UUAUUCAAGGGAAAGUAGGAAGC | 1153 | 2467-2489 |
| AD-1736214 | CAGAACUGAAUGCUCAAGUCA | 885 | 420-440 | UGACUUGAGCAUUCAGUUCUGGA | 1154 | 418-440 |
| AD-1736215 | UCUUGUUACUAAUUUCUCAUA | 886 | 3945-3965 | UAUGAGAAAUUAGUAACAAGAAC | 1155 | 3943-3965 |
| AD-1736216 | CUCAGUGAUAGCCUUUAUACA | 887 | 8586-8606 | UGUAUAAAGGCUAUCACUGAGUU | 1156 | 8584-8606 |
| AD-1736217 | UUUUAUGCUCCUAAAACAUCA | 888 | 3065-3085 | UGAUGUUUUAGGAGCAUAAAAGA | 1157 | 3063-3085 |
| AD-1736218 | UUCUCCUUAGGUUAUGUUCAA | 889 | 4551-4571 | UUGAACAUAACCUAAGGAGAAUU | 1158 | 4549-4571 |
| AD-1736219 | AUUGCUCAUCGAGACAUAAAA | 890 | 1175-1195 | UUUUAUGUCUCGAUGAGCAAUAG | 1159 | 1173-1195 |
| AD-1736220 | CCUUUGUACAUACACACUUAA | 891 | 2746-2766 | UUAAGUGUGUAUGUACAAAGGAA | 1160 | 2744-2766 |
| AD-1736221 | UUGAACCAAGAGCACAUGAAA | 892 | 2025-2045 | UUUCAUGUGCUCUUGGUUCAAGU | 1161 | 2023-2045 |
| AD-1736222 | UCAGGGAUUUUAAAGUCUAAA | 893 | 3668-3688 | UUUAGACUUUAAAAUCCCUGAGG | 1162 | 3666-3688 |
| AD-1736223 | UACUUGCUUAUAAAGUGAUAA | 894 | 3153-3173 | UUAUCACUUUAUAAGCAAGUACC | 1163 | 3151-3173 |
| AD-1736224 | AUGCAUCUAAACCUACCUUGA | 895 | 6145-6165 | UCAAGGUAGGUUUAGAUGCAUGC | 1164 | 6143-6165 |
| AD-1736225 | UUGGGAAAACACUAUUAUGAA | 896 | 4629-4649 | UUCAUAAUAGUGUUUUCCCAAGU | 1165 | 4627-4649 |
| AD-1736226 | AAACUCCAAAGAGAAAUGAAA | 897 | 8456-8476 | UUUCAUUUCUCUUUGGAGUUUGA | 1166 | 8454-8476 |
| AD-1736227 | AUGGGCAUUUUUCAAAACAUA | 898 | 5758-5778 | UAUGUUUUGAAAAAUGCCCAUAU | 1167 | 5756-5778 |
| AD-1736228 | AUAAUGGAACUUGGACUCAAA | 899 | 996-1016 | UUUGAGUCCAAGUUCCAUUAUCU | 1168 | 994-1016 |
| AD-1736229 | UGUGUAGAUGACGAAACCAAA | 900 | 6008-6028 | UUUGGUUUCGUCAUCUACACAAA | 1169 | 6006-6028 |
| AD-1736230 | UUCUACCUCAAAGAUAAGACA | 901 | 1790-1810 | UGUCUUAUCUUUGAGGUAGAACA | 1170 | 1788-1810 |
| AD-1736231 | AGAAGGCAUAUUGAGUUAUUA | 902 | 2536-2556 | UAAUAACUCAAUAUGCCUUCUAA | 1171 | 2534-2556 |
| AD-1736232 | AUGGAGUUUUUCUGCUAUUUA | 903 | 5629-5649 | UAAAUAGCAGAAAAACUCCAUGU | 1172 | 5627-5649 |
| AD-1736233 | UCAGUUUCUCAUUAGUUUGUA | 904 | 6558-6578 | UACAAACUAAUGAGAAACUGAGG | 1173 | 6556-6578 |
| AD-1736234 | CUGCAUAUAUAAGAACGAAAA | 905 | 3976-3996 | UUUUCGUUCUUAUAUAUGCAGAA | 1174 | 3974-3996 |
| AD-1736235 | UGAGCCAAAAUAUUAAAUUCA | 906 | 7927-7947 | UGAAUUUAAUAUUUUGGCUCAGA | 1175 | 7925-7947 |
| AD-1736236 | AGAAUGAUAUUUCCUGUUUUA | 907 | 4035-4055 | UAAAACAGGAAAUAUCAUUCUUG | 1176 | 4033-4055 |
| AD-1736237 | GUGCUCUAUAUAAAUCUUCCA | 908 | 7385-7405 | UGGAAGAUUUAUAUAGAGCACAA | 1177 | 7383-7405 |
| AD-1736238 | UUUACAGAGAAGCAUCUUAUA | 909 | 7468-7488 | UAUAAGAUGCUUCUCUGUAAAAU | 1178 | 7466-7488 |
| AD-1736239 | GUCUUGAGUUCUGCAUGACAA | 910 | 3761-3781 | UUGUCAUGCAGAACUCAAGACAU | 1179 | 3759-3781 |
| AD-1736240 | CUUCAAAUCUAUAAUUUUACA | 911 | 3017-3037 | UGUAAAAUUAUAGAUUUGAAGGG | 1180 | 3015-3037 |
| AD-1736241 | GAAGCAUAUAGAACUCUAUUA | 912 | 8810-8830 | UAAUAGAGUUCUAUAUGCUUCAC | 1181 | 8808-8830 |
| AD-1736242 | GAGCUGCUUCUAAAUAACAAA | 913 | 5678-5698 | UUUGUUAUUUAGAAGCAGCUCUC | 1182 | 5676-5698 |
| AD-1736243 | UGACUGUAUUUCCUGAUCAUA | 914 | 7197-7217 | UAUGAUCAGGAAAUACAGUCACA | 1183 | 7195-7217 |
| AD-1736244 | GUUCUGAAGGAUAUACUUCUA | 915 | 3402-3422 | UAGAAGUAUAUCCUUCAGAACGC | 1184 | 3400-3422 |
| AD-1736245 | GUGAAGCAUGAUUCAAUACUA | 916 | 1256-1276 | UAGUAUUGAAUCAUGCUUCACAG | 1185 | 1254-1276 |
| AD-1736246 | AAUGCUGCUUCACAGAUUUUA | 917 | 474-494 | UAAAAUCUGUGAAGCAGCAUUCG | 1186 | 472-494 |
| AD-1736247 | UGUGGGUUAUGUUAAUCUGAA | 918 | 7283-7303 | UUCAGAUUAACAUAACCCACAGU | 1187 | 7281-7303 |
| AD-1736248 | GAGUACAGAAGAAUGCUCAUA | 919 | 2926-2946 | UAUGAGCAUUCUUCUGUACUCAC | 1188 | 2924-2946 |
| AD-1736249 | CUGUAUUUCAUCCUAGAUUUA | 920 | 7162-7182 | UAAAUCUAGGAUGAAAUACAGCU | 1189 | 7160-7182 |
| AD-1736250 | AAUCUGGUAAAUGCUGGAAAA | 921 | 695-715 | UUUUCCAGCAUUUACCAGAUUGC | 1190 | 693-715 |
| AD-1736251 | UGCUCAGAUUACCUGAUCGUA | 922 | 3293-3313 | UACGAUCAGGUAAUCUGAGCAAU | 1191 | 3291-3313 |
| AD-1736252 | GACAGCUAUGGAGUUUGCGUA | 923 | 3531-3551 | UACGCAAACUCCAUAGCUGUCAG | 1192 | 3529-3551 |
| AD-1736253 | UAGAACAUGCUCACUUACAAA | 924 | 4391-4411 | UUUGUAAGUGAGCAUGUUCUAUA | 1193 | 4389-4411 |
| AD-1736254 | AUUAUGCUUCAUUUCACUAUA | 925 | 3450-3470 | UAUAGUGAAAUGAAGCAUAAUCA | 1194 | 3448-3470 |
| AD-1736255 | CAACAGCAAGUCAUAAAAGUA | 926 | 8624-8644 | UACUUUUAUGACUUGCUGUUGGU | 1195 | 8622-8644 |
| AD-1736256 | UUGUCUGAAAAUGUCUUUAAA | 927 | 2702-2722 | UUUAAAGACAUUUUCAGACAAUG | 1196 | 2700-2722 |
| AD-1736257 | UGUUGGUUCAAAGGACAAUUA | 928 | 774-794 | UAAUUGUCCUUUGAACCAACAGA | 1197 | 772-794 |
| AD-1736258 | UCUAAACUUUAAUAUAUCGAA | 929 | 2190-2210 | UUCGAUAUAUUAAAGUUUAGAGC | 1198 | 2188-2210 |
| AD-1736259 | AAGUGAAACAUGACUGUCGUA | 930 | 4875-4895 | UACGACAGUCAUGUUUCACUUAA | 1199 | 4873-4895 |
| AD-1736260 | AUCGUCCAUACAGUUUUCAUA | 931 | 7719-7739 | UAUGAAAACUGUAUGGACGAUAU | 1200 | 7717-7739 |
| AD-1736261 | CUUCAGGAAAUAGUAGGAAAA | 932 | 809-829 | UUUUCCUACUAUUUCCUGAAGCA | 1201 | 807-829 |
| AD-1736262 | CACCCUUUCUGUAAGGACUGA | 933 | 6426-6446 | UCAGUCCUUACAGAAAGGGUGGG | 1202 | 6424-6446 |
| AD-1736263 | AAAGAGAUACCUGACAUCCUA | 934 | 2354-2374 | UAGGAUGUCAGGUAUCUCUUUUG | 1203 | 2352-2374 |
| AD-1736264 | AUGAGCAUUAAUGUUUUCUGA | 935 | 2647-2667 | UCAGAAAACAUUAAUGCUCAUUA | 1204 | 2645-2667 |
| AD-1736265 | UGACAUUUUUUCUCAUAGAAA | 936 | 2603-2623 | UUUCUAUGAGAAAAAAUGUCAAC | 1205 | 2601-2623 |
| AD-1736266 | CAGAGAUUAAAUGACUACUAA | 937 | 6032-6052 | UUAGUAGUCAUUUAAUCUCUGAU | 1206 | 6030-6052 |
| AD-1736267 | GCUGUCAAAUGUUAUAUUGUA | 938 | 4463-4483 | UACAAUAUAACAUUUGACAGCAU | 1207 | 4461-4483 |
| AD-1736268 | AAUAUAAUACUACAGCAAAAA | 939 | 7041-7061 | UUUUUGCUGUAGUAUUAUAUUCC | 1208 | 7039-7061 |
| AD-1736269 | GGUGUCUAAAUAUAAUUUCAA | 940 | 4153-4173 | UUGAAAUUAUAUUUAGACACCCU | 1209 | 4151-4173 |
| AD-1736270 | CUCUUUAUACAAAAUGAGAGA | 941 | 7903-7923 | UCUCUCAUUUUGUAUAAAGAGAA | 1210 | 7901-7923 |
| AD-1736271 | GCAGUACCAAAUGAAUAAAAA | 942 | 5410-5430 | UUUUUAUUCAUUUGGUACUGCUA | 1211 | 5408-5430 |
| AD-1736272 | UUGUUGGUACACAAGGUAAAA | 943 | 1149-1169 | UUUUACCUUGUGUACCAACAAUC | 1212 | 1147-1169 |
| AD-1736273 | CUCUUAUUUAACUUAACCGUA | 944 | 5917-5937 | UACGGUUAAGUUAAAUAAGAGUU | 1213 | 5915-5937 |
| AD-1736274 | UGCUUGAUGAUACAAUGAAUA | 945 | 1338-1358 | UAUUCAUUGUAUCAUCAAGCAUU | 1214 | 1336-1358 |
| AD-1736275 | AACUGGGAAAGACAAUUUGAA | 946 | 6920-6940 | UUCAAAUUGUCUUUCCCAGUUAC | 1215 | 6918-6940 |
| AD-1736276 | CUCCUUAUAUGACUAUUUGAA | 947 | 1048-1068 | UUCAAAUAGUCAUAUAAGGAGCC | 1216 | 1046-1068 |
| AD-1736277 | AGUCAUGCUAACCAAUGGAAA | 948 | 367-387 | UUUCCAUUGGUUAGCAUGACUGA | 1217 | 365-387 |
| AD-1736278 | CCUGAAGUCACACAGCUAAUA | 949 | 2391-2411 | UAUUAGCUGUGUGACUUCAGGAC | 1218 | 2389-2411 |
| AD-1736279 | UUGCUGCUGACAACAAAGAUA | 950 | 978-998 | UAUCUUUGUUGUCAGCAGCAAUG | 1219 | 976-998 |
| AD-1736280 | UACUGACUUUGCCUGUAAAGA | 951 | 7757-7777 | UCUUUACAGGCAAAGUCAGUAAU | 1220 | 7755-7777 |
| AD-1736281 | AAUCCCUAUUUUAAAAUUCCA | 952 | 7070-7090 | UGGAAUUUUAAAAUAGGGAUUGU | 1221 | 7068-7090 |
| AD-1736282 | GUGCCUUAGGAGAUUACCCUA | 953 | 7330-7350 | UAGGGUAAUCUCCUAAGGCACUC | 1222 | 7328-7350 |
| AD-1736283 | GACAAUCUAAAUAUAUCAUCA | 954 | 3857-3877 | UGAUGAUAUAUUUAGAUUGUCUG | 1223 | 3855-3877 |
| AD-1736284 | UGUGAUAAUGAACACCGUAAA | 955 | 2438-2458 | UUUACGGUGUUCAUUAUCACAAG | 1224 | 2436-2458 |
| AD-1736285 | UCACCGUUGUAAAGACUUUUA | 956 | 8242-8262 | UAAAAGUCUUUACAACGGUGAUU | 1225 | 8240-8262 |
| AD-1736286 | UUAGAGAAGCUUCCUGACAUA | 957 | 6402-6422 | UAUGUCAGGAAGCUUCUCUAAUC | 1226 | 6400-6422 |
| AD-1736287 | AACAGUAAAGAAAUGCUACUA | 958 | 3252-3272 | UAGUAGCAUUUCUUUACUGUUAU | 1227 | 3250-3272 |
| AD-1736288 | UAUGUUCUGACUUGAGAGUUA | 959 | 4855-4875 | UAACUCUCAAGUCAGAACAUAAA | 1228 | 4853-4875 |
| AD-1736289 | UUAGAGCAUGCUAUCUUUAGA | 960 | 2109-2129 | UCUAAAGAUAGCAUGCUCUAAUU | 1229 | 2107-2129 |
| AD-1736290 | UAGUCUUUGAUUGAAAUAAGA | 961 | 5314-5334 | UCUUAUUUCAAUCAAAGACUAUG | 1230 | 5312-5334 |
| AD-1736291 | UUGGUAAACGAGAUUUAACAA | 962 | 3038-3058 | UUGUUAAAUCUCGUUUACCAAAG | 1231 | 3036-3058 |
| AD-1736292 | GUCUGAAAAUUGCUUUCAUUA | 963 | 3798-3818 | UAAUGAAAGCAAUUUUCAGACCU | 1232 | 3796-3818 |
| AD-1736293 | AUGAACUUGUUGCCUUGUAAA | 964 | 4741-4761 | UUUACAAGGCAACAAGUUCAUGC | 1233 | 4739-4761 |
| AD-1736294 | GGGCAAUAAACUGUAUCAAAA | 965 | 8379-8399 | UUUUGAUACAGUUUAUUGCCCAA | 1234 | 8377-8399 |
| AD-1736295 | ACAUGUUAGCAUAUAAUGUAA | 966 | 3175-3195 | UUACAUUAUAUGCUAACAUGUAC | 1235 | 3173-3195 |
| AD-1736296 | UGUGAGAGUAUAGAAUUUCUA | 967 | 6367-6387 | UAGAAAUUCUAUACUCUCACAGU | 1236 | 6365-6387 |
| AD-1736297 | AUCCUUCAUUUGGCACUAUAA | 968 | 6764-6784 | UUAUAGUGCCAAAUGAAGGAUUA | 1237 | 6762-6784 |
| AD-1736298 | ACAUUUAGAAAGUAGCUUUAA | 969 | 3824-3844 | UUAAAGCUACUUUCUAAAUGUGU | 1238 | 3822-3844 |
| AD-1736299 | AACUGUUCUAUAAAGCAAAGA | 970 | 3364-3384 | UCUUUGCUUUAUAGAACAGUUAA | 1239 | 3362-3384 |
| AD-1736300 | CCUCCAGAGAUGAAAGAUCUA | 971 | 894-914 | UAGAUCUUUCAUCUCUGGAGGAG | 1240 | 892-914 |
| AD-1736301 | UUGUUUAACUAUGACUCCUAA | 972 | 4249-4269 | UUAGGAGUCAUAGUUAAACAACA | 1241 | 4247-4269 |
| AD-1736302 | CUGUUUGACAAUGCUUUGUUA | 973 | 5585-5605 | UAACAAAGCAUUGUCAAACAGAA | 1242 | 5583-5605 |
| AD-1736303 | UGUCCUAAAAGAAAUUUUUCA | 974 | 7227-7247 | UGAAAAAUUUCUUUUAGGACAAC | 1243 | 7225-7247 |
| AD-1736304 | ACAGAUUGAACAAAGAACUUA | 975 | 7572-7592 | UAAGUUCUUUGUUCAAUCUGUAG | 1244 | 7570-7592 |
| AD-1736305 | GUUUAACCUGUCCAAACUUCA | 976 | 6673-6693 | UGAAGUUUGGACAGGUUAAACGA | 1245 | 6671-6693 |
| AD-1736306 | CAAAAUGUUUAACUUUACCAA | 977 | 8606-8626 | UUGGUAAAGUUAAACAUUUUGGU | 1246 | 8604-8626 |
| AD-1736307 | UCCCAUUGUGUAAUAUUUAUA | 978 | 4494-4514 | UAUAAAUAUUACACAAUGGGACU | 1247 | 4492-4514 |
| AD-1736308 | GUUGGGUAAAUAUGCUUAUUA | 979 | 4065-4085 | UAAUAAGCAUAUUUACCCAACAU | 1248 | 4063-4085 |
| AD-1736309 | CAGGGUUGUCUUUGAGUCUGA | 980 | 5187-5207 | UCAGACUCAAAGACAACCCUGAG | 1249 | 5185-5207 |
| AD-1736310 | GCUAAAAUCCAACUGCAAUUA | 981 | 7425-7445 | UAAUUGCAGUUGGAUUUUAGCCC | 1250 | 7423-7445 |
| AD-1736311 | CAGGACUGAAGUGUGUAUGUA | 982 | 291-311 | UACAUACACACUUCAGUCCUGGC | 1251 | 289-311 |
| AD-1736312 | GAGUACCAAUUGCCUUAUUAA | 983 | 1457-1477 | UUAAUAAGGCAAUUGGUACUCCU | 1252 | 1455-1477 |
| AD-1736313 | AAGUGCCUAUGUGAAGUGAUA | 984 | 3886-3906 | UAUCACUUCACAUAGGCACUUUU | 1253 | 3884-3906 |
| AD-1736314 | UAACAAUAAUAGAACUGCCAA | 985 | 7092-7112 | UUGGCAGUUCUAUUAUUGUUAGG | 1254 | 7090-7112 |
| AD-1736315 | GCUGUUCCAUACCAUUGCUUA | 986 | 6978-6998 | UAAGCAAUGGUAUGGAACAGCAG | 1255 | 6976-6998 |
| AD-1736316 | UAGACUGGAGAAGAUUAUUCA | 987 | 7997-8017 | UGAAUAAUCUUCUCCAGUCUAGA | 1256 | 7995-8017 |
| AD-1736317 | UUCCAACAGCAUCACCAAAUA | 988 | 516-536 | UAUUUGGUGAUGCUGUUGGAAGG | 1257 | 514-536 |
| AD-1736318 | UAAUUGGUACAGAUUCUGUUA | 989 | 4779-4799 | UAACAGAAUCUGUACCAAUUACA | 1258 | 4777-4799 |
| AD-1736319 | GAGAAACAUUAUUUGUUGUCA | 990 | 4710-4730 | UGACAACAAAUAAUGUUUCUCUU | 1259 | 4708-4730 |
| AD-1736320 | CGCUCUCAAUUGCUAGUGGUA | 991 | 1107-1127 | UACCACUAGCAAUUGAGAGCGCC | 1260 | 1105-1127 |
| AD-1736321 | CGGUAUCAGAAACAGCAAAUA | 992 | 2873-2893 | UAUUUGCUGUUUCUGAUACCGUC | 1261 | 2871-2893 |
| AD-1736322 | CCCAGACAUAAUUUUAUAUUA | 993 | 7449-7469 | UAAUAUAAAAUUAUGUCUGGGAG | 1262 | 7447-7469 |
| AD-1736323 | UAGUACAUUUUGAGGUAUUUA | 994 | 4215-4235 | UAAAUACCUCAAAAUGUACUAAC | 1263 | 4213-4235 |
| AD-1736324 | AAAUGCUAUUGAUAACAGUAA | 995 | 3211-3231 | UUACUGUUAUCAAUAGCAUUUAC | 1264 | 3209-3231 |
| AD-1736325 | UAGCUGAAUUUGGGACUAUGA | 996 | 6490-6510 | UCAUAGUCCCAAAUUCAGCUAUC | 1265 | 6488-6510 |
| AD-1736326 | AUUGUGGUUUUCAAAGAUAUA | 997 | 5535-5555 | UAUAUCUUUGAAAACCACAAUGG | 1266 | 5533-5555 |
| AD-1736327 | CCUGGUUGCAGUAUCUGAAAA | 998 | 4985-5005 | UUUUCAGAUACUGCAACCAGGUU | 1267 | 4983-5005 |
| AD-1736328 | UAGAAUGGUUGUUGAGCUAAA | 999 | 6297-6317 | UUUAGCUCAACAACCAUUCUACA | 1268 | 6295-6317 |
| AD-1736329 | CUGGCCAUCAUUAUUACUGUA | 1000 | 560-580 | UACAGUAAUAAUGAUGGCCAGCU | 1269 | 558-580 |
| AD-1736330 | UUGACAGAUAAAAUACGAAGA | 1001 | 3735-3755 | UCUUCGUAUUUUAUCUGUCAAUU | 1270 | 3733-3755 |
| AD-1736331 | UCACAAACACAUCAUUACAAA | 1002 | 5473-5493 | UUUGUAAUGAUGUGUUUGUGAAU | 1271 | 5471-5493 |
| AD-1736332 | CCAUGAUUGUAUGAAAAUAGA | 1003 | 4962-4982 | UCUAUUUUCAUACAAUCAUGGAA | 1272 | 4960-4982 |
| AD-1736333 | CACAGAGUAAUGAAUAUUUAA | 1004 | 2978-2998 | UUAAAUAUUCAUUACUCUGUGUC | 1273 | 2976-2998 |
| AD-1736334 | AGCUGCUUAGUGGAAGAUGUA | 1005 | 2222-2242 | UACAUCUUCCACUAAGCAGCUCC | 1274 | 2220-2242 |
| AD-1736335 | UAGCUCUGUGUGCUGAUACCA | 1006 | 5606-5626 | UGGUAUCAGCACACAGAGCUAGA | 1275 | 5604-5626 |
| AD-1736336 | CAUUCGAGAGAAUAUGAGCUA | 1007 | 7144-7164 | UAGCUCAUAUUCUCUCGAAUGUA | 1276 | 7142-7164 |
| AD-1736337 | AUGCAUUUUCUGAAAAUGUAA | 1008 | 8734-8754 | UUACAUUUUCAGAAAAUGCAUAC | 1277 | 8732-8754 |
| AD-1736338 | GUUUCGACCAAGUAUCCCAAA | 1009 | 1537-1557 | UUUGGGAUACUUGGUCGAAACUU | 1278 | 1535-1557 |
| AD-1736339 | UCUUGCUUGCCACAAUAUCGA | 1010 | 3610-3630 | UCGAUAUUGUGGCAAGCAAGAUA | 1279 | 3608-3630 |
| AD-1736340 | GGGUACAAGAGGGAAUACAAA | 1011 | 3629-3649 | UUUGUAUUCCCUCUUGUACCCGA | 1280 | 3627-3649 |
| AD-1736341 | GGUGAUCAAAUCCUGUGUCUA | 1012 | 394-414 | UAGACACAGGAUUUGAUCACCUG | 1281 | 392-414 |
| AD-1736342 | CAGUGGGUUUCUAGGAUAGGA | 1013 | 3779-3799 | UCCUAUCCUAGAAACCCACUGUC | 1282 | 3777-3799 |
| AD-1736343 | AACUAAGUAUUUUUAAGGACA | 1014 | 7869-7889 | UGUCCUUAAAAAUACUUAGUUAC | 1283 | 7867-7889 |
| AD-1736344 | CAUAAUGUGAUUGUUAAAUUA | 1015 | 6951-6971 | UAAUUUAACAAUCACAUUAUGGG | 1284 | 6949-6971 |
| AD-1736345 | GAGAAGAAUUAAGAAAACUCA | 1016 | 2820-2840 | UGAGUUUUCUUAAUUCUUCUCAA | 1285 | 2818-2840 |
| AD-1736346 | GAAGAGAAAUUGAUUGUUUAA | 1017 | 7635-7655 | UUAAACAAUCAAUUUCUCUUCUC | 1286 | 7633-7655 |
| AD-1736347 | AGUGUGAAACUUGUGCCAUAA | 1018 | 1218-1238 | UUAUGGCACAAGUUUCACACUUU | 1287 | 1216-1238 |
| AD-1736348 | CUGAGACACUGAGAAAUGUCA | 1019 | 2372-2392 | UGACAUUUCUCAGUGUCUCAGGA | 1288 | 2370-2392 |
| AD-1736349 | UGGCUUAUAUGUGUUUAAAGA | 1020 | 7488-7508 | UCUUUAAACACAUAUAAGCCAAU | 1289 | 7486-7508 |
| AD-1736350 | UGCAUUUUGACAUUGCAAAAA | 1021 | 8188-8208 | UUUUUGCAAUGUCAAAAUGCAAU | 1290 | 8186-8208 |
| AD-1736351 | UUAGGUUAGAAGUUCCAGAAA | 1022 | 5053-5073 | UUUCUGGAACUUCUAACCUAAUG | 1291 | 5051-5073 |
| AD-1736352 | UGCGACAUGAAAACAUCCUUA | 1023 | 951-971 | UAAGGAUGUUUUCAUGUCGCAGC | 1292 | 949-971 |
| AD-1736353 | GAGAGGAUUUUUUACCAUCUA | 1024 | 4095-4115 | UAGAUGGUAAAAAAUCCUCUCUU | 1293 | 4093-4115 |
| AD-1736354 | GAAGGUGAAGAGAAUUUCAAA | 1025 | 8438-8458 | UUUGAAAUUCUCUUCACCUUCCA | 1294 | 8436-8458 |
| AD-1736355 | UGGUAUCUGAAUAUCAUGAAA | 1026 | 1023-1043 | UUUCAUGAUAUUCAGAUACCAGC | 1295 | 1021-1043 |
| AD-1736356 | UGUUGGUUUAAUUUUUCAACA | 1027 | 7658-7678 | UGUUGAAAAAUUAAACCAACAUG | 1296 | 7656-7678 |
| AD-1736357 | UAGUUGUAAUUUAAAUGUGGA | 1028 | 8419-8439 | UCCACAUUUAAAUUACAACUACU | 1297 | 8417-8439 |
| AD-1736358 | GUGAAUGUUUUUGCCAUUUUA | 1029 | 1763-1783 | UAAAAUGGCAAAAACAUUCACAU | 1298 | 1761-1783 |
| AD-1736359 | CAGAGAUGAUUUUUCUUUUAA | 1030 | 6800-6820 | UUAAAAGAAAAAUCAUCUCUGCC | 1299 | 6798-6820 |
| AD-1736360 | AGAAAAUUAGAUUCAGAUCUA | 1031 | 8058-8078 | UAGAUCUGAAUCUAAUUUUCUGU | 1300 | 8056-8078 |
| AD-1736361 | ACUGGAAAAUUAAGAAAGUAA | 1032 | 5390-5410 | UUACUUUCUUAAUUUUCCAGUGG | 1301 | 5388-5410 |
| AD-1736362 | UAAAGUGGGAAAGAUAAUAAA | 1033 | 8762-8782 | UUUAUUAUCUUUCCCACUUUACA | 1302 | 8760-8782 |
| AD-1736363 | CUAGGCUAAGAAAGAGUUGUA | 1034 | 6278-6298 | UACAACUCUUUCUUAGCCUAGUU | 1303 | 6276-6298 |
| AD-1736364 | GCUGAUAAAAUUAAUGGAUAA | 1035 | 6335-6355 | UUAUCCAUUAAUUUUAUCAGCAC | 1304 | 6333-6355 |
| AD-1736365 | CCUGUAUUUCUUUAGUUGUCA | 1036 | 6653-6673 | UGACAACUAAAGAAAUACAGGAA | 1305 | 6651-6673 |
| AD-1736366 | UUUGUUUUGCUUUUGACAAAA | 1037 | 6996-7016 | UUUUGUCAAAAGCAAAACAAAGC | 1306 | 6994-7016 |
| AD-1736367 | UGGCUGUGAAAAUAUUCUCCA | 1038 | 876-896 | UGGAGAAUAUUUUCACAGCCACA | 1307 | 874-896 |
| AD-1736368 | ACUAUUUUAGGCAUAGCACUA | 1039 | 4897-4917 | UAGUGCUAUGCCUAAAAUAGUGC | 1308 | 4895-4917 |
| AD-1736369 | UUUCAGUACUGUAUAAAGUGA | 1040 | 4651-4671 | UCACUUUAUACAGUACUGAAAAU | 1309 | 4649-4671 |
| AD-1736370 | GUGAUGCAAUCUAUGUUUCCA | 1041 | 2129-2149 | UGGAAACAUAGAUUGCAUCACCU | 1310 | 2127-2149 |
| AD-1736371 | UGUUUGGCAGAUCUCAUCAAA | 1042 | 6613-6633 | UUUGAUGAGAUCUGCCAAACAUU | 1311 | 6611-6633 |
| AD-1736372 | AAGGUUGUUUGUGACCAGAAA | 1043 | 1517-1537 | UUUCUGGUCACAAACAACCUUUC | 1312 | 1515-1537 |
| AD-1736373 | GGAAGGAAAUAUUUUGAGAAA | 1044 | 2794-2814 | UUUCUCAAAAUAUUUCCUUCCUU | 1313 | 2792-2814 |
| AD-1736374 | UUUUGGUAGCGUGACUCUUCA | 1045 | 8132-8152 | UGAAGAGUCACGCUACCAAAAGA | 1314 | 8130-8152 |
| AD-1736375 | UGAACAGGAAAAGAUGUAAAA | 1046 | 2045-2065 | UUUUACAUCUUUUCCUGUUCAUU | 1315 | 2043-2065 |
| AD-1736376 | GGGAACCAAGAGGUAUAUGGA | 1047 | 1309-1329 | UCCAUAUACCUCUUGGUUCCCAC | 1316 | 1307-1329 |
| AD-1736377 | UGGCACUAGGUCCAAAUCUUA | 1048 | 2419-2439 | UAAGAUUUGGACCUAGUGCCAGU | 1317 | 2417-2439 |
| AD-1736378 | CACACCUUCAUAUGGAGAUUA | 1049 | 1131-1151 | UAAUCUCCAUAUGAAGGUGUGCC | 1318 | 1129-1151 |
| AD-1736379 | CUGGUUUACUGGGAAAUAGCA | 1050 | 1406-1426 | UGCUAUUUCCCAGUAAACCAGAC | 1319 | 1404-1426 |
| AD-1736380 | GCCCAAAGGAAUGUAUAUAAA | 1051 | 8316-8336 | UUUAUAUACAUUCCUUUGGGCCU | 1320 | 8314-8336 |
| AD-1736381 | UUAACUGCAAGAGUUUACUGA | 1052 | 7265-7285 | UCAGUAAACUCUUGCAGUUAAAA | 1321 | 7263-7285 |
| AD-1736382 | GAACCACUCUCUGAGUGCAAA | 1053 | 677-697 | UUUGCACUCAGAGAGUGGUUCCU | 1322 | 675-697 |
| AD-1736383 | UUAUCUUUUCAUCCUGGCAUA | 1054 | 7607-7627 | UAUGCCAGGAUGAAAAGAUAAAG | 1323 | 7605-7627 |
| AD-1736384 | GUGAGUGUUGGUAUGCCAACA | 1055 | 1605-1625 | UGUUGGCAUACCAACACUCACGC | 1324 | 1603-1625 |
| AD-1736385 | AGAAUGUAUUUGGCUAAAUAA | 1056 | 5338-5358 | UUAUUUAGCCAAAUACAUUCUCA | 1325 | 5336-5358 |
| AD-1736386 | GAACCCUUUUAUUAAGAGGAA | 1057 | 3317-3337 | UUCCUCUUAAUAAAAGGGUUCCA | 1326 | 3315-3337 |
| AD-1736387 | CUCCUGUCCAUAGCUGCGAUA | 1058 | 590-610 | UAUCGCAGCUAUGGACAGGAGGC | 1327 | 588-610 |
| AD-1736388 | ACUGAGUUGAAAUAAGGAAGA | 1059 | 3501-3521 | UCUUCCUUAUUUCAACUCAGUAA | 1328 | 3499-3521 |
| AD-1736389 | CUGCCAAACAGAAGGAGCAUA | 1060 | 337-357 | UAUGCUCCUUCUGUUUGGCAGGU | 1329 | 335-357 |
| AD-1736390 | GGCAAAGUUGUGAAGCACUCA | 1061 | 1563-1583 | UGAGUGCUUCACAACUUUGCCAC | 1330 | 1561-1583 |
| AD-1736391 | AUAGGAUGAUGAAGUUUAGAA | 1062 | 4413-4433 | UUCUAAACUUCAUCAUCCUAUUU | 1331 | 4411-4433 |
| AD-1736392 | AUUCGUAUCUUAAAAUGGCAA | 1063 | 5513-5533 | UUGCCAUUUUAAGAUACGAAUAC | 1332 | 5511-5533 |
| AD-1736393 | CACAGCUUGGGUAAUAGCGUA | 1064 | 3384-3404 | UACGCUAUUACCCAAGCUGUGCU | 1333 | 3382-3404 |
| AD-1736394 | UUUGCAACAACAUAACACUGA | 1065 | 492-512 | UCAGUGUUAUGUUGUUGCAAAAA | 1334 | 490-512 |
| AD-1736395 | UGCCACCAUGUGACUUAUUGA | 1066 | 5153-5173 | UCAAUAAGUCACAUGGUGGCAAA | 1335 | 5151-5173 |
| AD-1736396 | UCGAUAGAGGAAAUGAGAAAA | 1067 | 1499-1519 | UUUUCUCAUUUCCUCUAUCGAGG | 1336 | 1497-1519 |
| AD-1736397 | UUUUUAAGUUAGCAGGACUUA | 1068 | 2152-2172 | UAAGUCCUGCUAACUUAAAAAGG | 1337 | 2150-2172 |
| AD-1736398 | UGUAAAUAAUUAUCUGCCUAA | 1069 | 8696-8716 | UUAGGCAGAUAAUUAUUUACAUC | 1338 | 8694-8716 |
| AD-1736399 | UGGUUGUACGUGCCUCAAAUA | 1070 | 4010-4030 | UAUUUGAGGCACGUACAACCAUA | 1339 | 4008-4030 |
| AD-1736400 | CCACACUGACUAGAGCCAACA | 2147 | 70-90 | UGUUGGCUCUAGUCAGUGUGGGC | 2148 | 68-90 |

TABLE 7
Modified Sense and Antisense Strand Sequences of ACVRIC dsRNA
Agents Comprising a GalNAc Derivative Targeting Ligand

| | | SEQ | | SEQ | | SEQ |
|---|---|---|---|---|---|---|
| Duplex | Antisense Sequence | ID | Antisense Sequence | ID | mRNA Target Sequence | ID |
| Name | 5′ to 3′ | NO: | 5′ to 3′ | NO: | 5′ to 3′ | NO: |
| AD- | ususuuuuCfaAfAfCfuccauuc | 2149 | VPusAfsggaAfuGfGfaguuUfgAfaaa | 1609 | UCUUUUUUCAAACUCCAUUC | 1878 |
| 1736131 | csusa | | aasgsa | | CUU | |
| AD- | ascsuuucAfuCfUfGfucuucag | 2150 | VPusAfsucuGfaAfGfacagAfuGfaaa | 1610 | UAACUUUCAUCUGUCUUCAG | 1879 |
| 1736132 | asusa | | gususa | | AUG | |
| AD- | cscsucauAfaCfUfUfcuccagu | 2151 | VPusUfsuacUfgGfAfgaagUfuAfuga | 1611 | CACCUCAUAACUUCUCCAGU | 1880 |
| 1736133 | asasa | | ggsusg | | AAU | |
| AD- | csuscacuAfuCfUfUfucaacau | 2152 | VPusUfsaauGfuUfGfaaagAfuAfgug | 1612 | CCCUCACUAUCUUUCAACAU | 1881 |
| 1736134 | usasa | | agsgsg | | UAU | |
| AD- | csuscguuAfaUfGfCfucucauc | 2153 | VPusUfsggaUfgAfGfagcaUfuAfacg | 1613 | UACUCGUUAAUGCUCUCAUC | 1882 |
| 1736135 | csasa | | agsusa | | CAC | |
| AD- | gsusucuuCfaUfAfAfuccacua | 2154 | VPusAfsguaGfuGfGfauuaUfgAfaga | 1614 | CUGUUCUUCAUAAUCCACUA | 1883 |
| 1736136 | csusa | | acsasg | | CUG | |
| AD- | gsusugacUfuCfAfUfccaaucu | 2155 | VPusAfsgagAfuUfGfgaugAfaGfuca | 1615 | GAGUUGACUUCAUCCAAUCU | 1884 |
| 1736137 | csusa | | acsusc | | CUA | |
| AD- | csusacacAfaUfGfAfacuucuu | 2156 | VPusUfsuaaGfaAfGfuucaUfuGfugu | 1616 | UUCUACACAAUGAACUUCUU | 1885 |
| 1736138 | asasa | | agsasa | | AAC | |
| AD- | csascaucUfaGfAfAfuucuuaa | 2157 | VPusAfsauuAfaGfAfauucUfaGfaug | 1617 | GUCACAUCUAGAAUUCUUAA | 1886 |
| 1736139 | ususa | | ugsasc | | UUU | |
| AD- | asasaucuCfuCfAfUfagcuuuc | 2158 | VPusAfsagaAfaGfCfuaugAfgAfgau | 1618 | AGAAAUCUCUCAUAGCUUUC | 1887 |
| 1736140 | ususa | | uuscsu | | UUU | |
| AD- | cscsuacuAfuUfGfUfagaauua | 2159 | VPusAfsguaAfuUfCfuacaAfuAfgua | 1619 | CACCUACUAUUGUAGAAUUA | 1888 |
| 1736141 | csusa | | ggsusg | | CUA | |
| AD- | asuscuucUfuUfUfAfgugcauu | 2160 | VPusUfsuaaUfgCfAfcuaaAfaGfaag | 1620 | UCAUCUUCUUUUAGUGCAUU | 1889 |
| 1736142 | asasa | | ausgsa | | AAA | |
| AD- | csuscaugUfaCfUfCfuucugau | 2161 | VPusGfsaauCfaGfAfagagUfaCfaug | 1621 | AGCUCAUGUACUCUUCUGAU | 1890 |
| 1736143 | uscsa | | agscsu | | UCU | |
| AD- | ususucucUfuUfGfUfaccuugg | 2162 | VPusUfsuccAfaGfGfuacaAfaGfaga | 1622 | UCUUUCUCUUUGUACCUUGG | 1891 |
| 1736144 | asasa | | aasgsa | | AAU | |
| AD- | usgsgcuuAfuAfCfAfucuucag | 2163 | VPusUfsucuGfaAfGfauguAfuAfagc | 1623 | UCUGGCUUAUACAUCUUCAG | 1892 |
| 1736145 | asasa | | casgsa | | AAA | |
| AD- | csasaaacAfaAfAfCfuacuccu | 2164 | VPusAfsuagGfaGfUfaguuUfuGfuuu | 1624 | AACAAAACAAAACUACUCCU | 1893 |
| 1736146 | asusa | | ugsusu | | AUU | |
| AD- | ascsuagcAfgAfAfCfucuuaug | 2165 | VPusUfsucaUfaAfGfaguuCfuGfcua | 1625 | UUACUAGCAGAACUCUUAUG | 1894 |
| 1736147 | asasa | | gusasa | | AAA | |
| AD- | cscsaaguCfaCfUfCfuauaauu | 2166 | VPusGfsgaaUfuAfUfagagUfgAfcuu | 1626 | UCCCAAGUCACUCUAUAAUU | 1895 |
| 1736148 | cscsa | | ggsgsa | | CCU | |
| AD- | uscscauuUfuUfCfUfugucauu | 2167 | VPusAfsuaaUfgAfCfaagaAfaAfaug | 1627 | UCUCCAUUUUUCUUGUCAUU | 1896 |
| 1736149 | asusa | | gasgsa | | AUG | |
| AD- | csasacacCfuCfAfAfcucaucu | 2168 | VPusAfsaagAfuGfAfguugAfgGfugu | 1628 | AGCAACACCUCAACUCAUCU | 1897 |
| 1736150 | ususa | | ugscsu | | UUU | |
| AD- | ascsucugAfaAfGfAfucugauu | 2169 | VPusUfsaaaUfcAfGfaucuUfuCfaga | 1629 | AAACUCUGAAAGAUCUGAUU | 1898 |
| 1736151 | usasa | | gususu | | UAU | |
| AD- | csusaguuCfuUfUfUfccgcaau | 2170 | VPusUfscauUfgCfGfgaaaAfgAfacu | 1630 | UUCUAGUUCUUUUCCGCAAU | 1899 |
| 1736152 | gsasa | | agsasa | | GAC | |
| AD- | csusuucaCfuCfUfGfaagaaau | 2171 | VPusGfsgauUfuCfUfucagAfgUfgaa | 1631 | AACUUUCACUCUGAAGAAAU | 1900 |
| 1736153 | cscsa | | agsusu | | CCG | |
| AD- | uscsuaacUfuUfUfCfucucuuu | 2172 | VPusAfsgaaAfgAfGfagaaAfaGfuua | 1632 | UAUCUAACUUUUCUCUCUUU | 1901 |
| 1736154 | csusa | | gasusa | | CUC | |
| AD- | uscsucaaCfuUfUfGfugucaaa | 2173 | VPusUfscuuUfgAfCfacaaAfgUfuga | 1633 | UAUCUCAACUUUGUGUCAAA | 1902 |
| 1736155 | gsasa | | gasusa | | GAA | |
| AD- | csusucaaUfaAfUfCfauuccuu | 2174 | VPusUfsaaaGfgAfAfugauUfaUfuga | 1634 | GUCUUCAAUAAUCAUUCCUU | 1903 |
| 1736156 | usasa | | agsasc | | UAA | |
| AD- | usasacugCfuCfUfUfcguauua | 2175 | VPusCfsuuaAfuAfCfgaagAfgCfagu | 1635 | CCUAACUGCUCUUCGUAUUA | 1904 |
| 1736157 | asgsa | | uasgsg | | AGA | |
| AD- | uscsuucuGfuCfAfUfaguucca | 2176 | VPusGfsuugGfaAfCfuaugAfcAfgaa | 1636 | AGUCUUCUGUCAUAGUUCCA | 1905 |
| 1736158 | ascsa | | gascsu | | ACA | |
| AD- | asuscucaGfaAfCfCfauaucug | 2177 | VPusAfsacaGfaUfAfugguUfcUfgag | 1637 | AAAUCUCAGAACCAUAUCUG | 1906 |
| 1736159 | ususa | | aususu | | UUG | |
| AD- | usgsaaucUfaUfCfUfucauuuu | 2178 | VPusGfsuaaAfaUfGfaagaUfaGfauu | 1638 | ACUGAAUCUAUCUUCAUUUU | 1907 |
| 1736160 | ascsa | | casgsu | | ACU | |
| AD- | ascscacuAfaAfCfUfuguuccu | 2179 | VPusAfsaagGfaAfCfaaguUfuAfgug | 1639 | GAACCACUAAACUUGUUCCU | 1908 |
| 1736161 | ususa | | gususc | | UUC | |
| AD- | asgsuguuCfuUfUfAfcuccuua | 2180 | VPusUfsguaAfgGfAfguaaAfgAfaca | 1640 | UCAGUGUUCUUUACUCCUUA | 1909 |
| 1736162 | csasa | | cusgsa | | CAG | |
| AD- | csusuuacUfcUfUfAfacaggau | 2181 | VPusUfsaauCfcUfGfuuaaGfaGfuaa | 1641 | UCCUUUACUCUUAACAGGAU | 1910 |
| 1736163 | usasa | | agsgsa | | UAU | |
| AD- | usasuaccUfaAfGfAfacauauu | 2182 | VPusGfsuaaUfaUfGfuucuUfaGfgua | 1642 | CUUAUACCUAAGAACAUAUU | 1911 |
| 1736164 | ascsa | | uasasg | | ACA | |
| AD- | csusucuaCfuGfAfGfaugaucc | 2183 | VPusUfsuggAfuCfAfucucAfgUfaga | 1643 | UUCUUCUACUGAGAUGAUCC | 1912 |
| 1736165 | asasa | | agsasa | | AAG | |
| AD- | csusguauUfuUfCfUfcauagag | 2184 | VPusUfsacuCfuAfUfgagaAfaAfuac | 1644 | UUCUGUAUUUUCUCAUAGAG | 1913 |
| 1736166 | usasa | | agsasa | | UAC | |
| AD- | ascsuuuuCfuCfUfUfucaguug | 2185 | VPusUfsacaAfcUfGfaaagAfgAfaaa | 1645 | AUACUUUUCUCUUUCAGUUG | 1914 |
| 1736167 | usasa | | gusasu | | UAG | |
| AD- | asusuaauUfuCfUfAfgucugug | 2186 | VPusUfsucaCfaGfAfcuagAfaAfuua | 1646 | UCAUUAAUUUCUAGUCUGUG | 1915 |
| 1736168 | asasa | | ausgsa | | AAA | |
| AD- | csuscuguAfuAfAfGfagguuuc | 2187 | VPusGfsugaAfaCfCfucuuAfuAfcag | 1647 | UUCUCUGUAUAAGAGGUUUC | 1916 |
| 1736169 | ascsa | | agsasa | | ACC | |
| AD- | gsascuuuCfaAfAfGfuacuaau | 2188 | VPusGfsuauUfaGfUfacuuUfgAfaag | 1648 | UUGACUUUCAAAGUACUAAU | 1917 |
| 1736170 | ascsa | | ucsasa | | ACU | |
| AD- | ususugccAfuUfAfUfacaaagu | 2189 | VPusAfsaacUfuUfGfuauaAfuGfgca | 1649 | UGUUUGCCAUUAUACAAAGU | 1918 |
| 1736171 | ususa | | aascsa | | UUG | |
| AD- | gsasacacUfaUfCfGfacauacc | 2190 | VPusGfsaggUfaUfGfucgaUfaGfugu | 1650 | CUGAACACUAUCGACAUACC | 1919 |
| 1736172 | uscsa | | ucsasg | | UCA | |
| AD- | usasgaauGfaCfAfUfuuacuaa | 2191 | VPusUfsauuAfgUfAfaaugUfcAfuuc | 1651 | CGUAGAAUGACAUUUACUAA | 1920 |
| 1736173 | usasa | | uascsg | | UAU | |
| AD- | asasgcuaCfuUfAfGfaaauguu | 2192 | VPusUfsaaaCfaUfUfucuaAfgUfagc | 1652 | ACAAGCUACUUAGAAAUGUU | 1921 |
| 1736174 | usasa | | uusgsu | | UAG | |
| AD- | cscsuuagUfaCfUfCfucagaga | 2193 | VPusAfsaucUfcUfGfagagUfaCfuaa | 1653 | UCCCUUAGUACUCUCAGAGA | 1922 |
| 1736175 | ususa | | ggsgsa | | UUA | |
| AD- | usgsaaugCfaCfAfCfuaacgua | 2194 | VPusAfsuuaCfgUfUfagugUfgCfauu | 1654 | CUUGAAUGCACACUAACGUA | 1923 |
| 1736176 | asusa | | casasg | | AUG | |
| AD- | asasgccuAfaUfGfAfugauaau | 2195 | VPusUfsaauUfaUfCfaucaUfuAfggc | 1655 | CAAAGCCUAAUGAUGAUAAU | 1924 |
| 1736177 | usasa | | uususg | | UAU | |
| AD- | asasacuuUfuUfAfCfuauccca | 2196 | VPusUfsaugGfgAfUfaguaAfaAfagu | 1656 | GGAAACUUUUUACUAUCCCA | 1925 |
| 1736178 | usasa | | uuscsc | | UAU | |
| AD- | csusagcuGfaUfAfCfucuuaag | 2197 | VPusUfsacuUfaAfGfaguaUfcAfgcu | 1657 | AGCUAGCUGAUACUCUUAAG | 1926 |
| 1736179 | usasa | | agscsu | | UAU | |
| AD- | asgscaugAfaAfAfGfauaacuc | 2198 | VPusUfsagaGfuUfAfucuuUfuCfaug | 1658 | GCAGCAUGAAAAGAUAACUC | 1927 |
| 1736180 | usasa | | cusgsc | | UAA | |
| AD- | usasagccAfuUfAfUfgcuauua | 2199 | VPusAfscuaAfuAfGfcauaAfuGfgcu | 1659 | ACUAAGCCAUUAUGCUAUUA | 1928 |
| 1736181 | gsusa | | uasgsu | | GUU | |
| AD- | uscsuagcUfuUfUfAfgcuaaca | 2200 | VPusUfsaugUfuAfGfcuaaAfaGfcua | 1660 | AUUCUAGCUUUUAGCUAACA | 1929 |
| 1736182 | usasa | | gasasu | | UAU | |
| AD- | gsusgugaUfuCfUfUfcaaacuu | 2201 | VPusUfsgaaGfuUfUfgaagAfaUfcac | 1661 | UUGUGUGAUUCUUCAAACUU | 1930 |
| 1736183 | csasa | | acsasa | | CAC | |
| AD- | uscsuccaGfaCfCfUfaacaguu | 2202 | VPusAfsaaaCfuGfUfuaggUfcUfgga | 1662 | UCUCUCCAGACCUAACAGUU | 1931 |
| 1736184 | ususa | | gasgsa | | UUA | |
| AD- | csusuuuuUfuCfAfUfcuagccu | 2203 | VPusCfsaagGfcUfAfgaugAfaAfaaa | 1663 | UUCUUUUUUUCAUCUAGCCU | 1932 |
| 1736185 | usgsa | | agsasa | | UGC | |
| AD- | csasaaauAfaUfUfCfuugacau | 2204 | VPusAfsgauGfuCfAfagaaUfuAfuuu | 1664 | GUCAAAAUAAUUCUUGACAU | 1933 |
| 1736186 | csusa | | ugsasc | | CUA | |
| AD- | gscsaucaCfuUfUfCfugaaaau | 2205 | VPusUfsuauUfuUfCfagaaAfgUfgau | 1665 | CAGCAUCACUUUCUGAAAAU | 1934 |
| 1736187 | asasa | | gcsusg | | AAG | |
| AD- | uscsuuuuGfuGfAfAfacauacu | 2206 | VPusAfsuagUfaUfGfuuucAfcAfaaa | 1666 | CAUCUUUUGUGAAACAUACU | 1935 |
| 1736188 | asusa | | gasusg | | AUU | |
| AD- | csusauuuCfuUfCfCfauaggcu | 2207 | VPusUfsuagCfcUfAfuggaAfgAfaau | 1667 | UUCUAUUUCUUCCAUAGGCU | 1936 |
| 1736189 | asasa | | agsasa | | AAA | |
| AD- | csusucaaUfgAfAfGfuguuaac | 2208 | VPusUfsuguUfaAfCfacuuCfaUfuga | 1668 | CUCUUCAAUGAAGUGUUAAC | 1937 |
| 1736190 | asasa | | agsasg | | AAC | |
| AD- | ususugccUfuCfAfUfucuacuu | 2209 | VPusGfsaaaGfuAfGfaaugAfaGfgca | 1669 | GUUUUGCCUUCAUUCUACUU | 1938 |
| 1736191 | uscsa | | aasasc | | UCU | |
| AD- | asgsguuuUfuAfAfAfuguccua | 2210 | VPusUfsuuaGfgAfCfauuuAfaAfaac | 1670 | UCAGGUUUUUAAAUGUCCUA | 1939 |
| 1736192 | asasa | | cusgsa | | AAA | |
| AD- | gsasauauCfuUfUfGfaguccuu | 2211 | VPusUfsgaaGfgAfCfucaaAfgAfuau | 1671 | GUGAAUAUCUUUGAGUCCUU | 1940 |
| 1736193 | csasa | | ucsasc | | CAA | |
| AD- | gsasacacUfuCfCfAfaagauua | 2212 | VPusAfsuuaAfuCfUfuuggAfaGfugu | 1672 | CUGAACACUUCCAAAGAUUA | 1941 |
| 1736194 | asusa | | ucsasg | | AUC | |
| AD- | csusaaauUfuUfGfUfcuaacaa | 2213 | VPusAfsuuuGfuUfAfgacaAfaAfuuu | 1673 | GCCUAAAUUUUGUCUAACAA | 1942 |
| 1736195 | asusa | | agsgsc | | AUG | |
| AD- | gsusaaacAfaCfUfUfaaaauug | 2214 | VPusAfsgcaAfuUfUfuaagUfuGfuuu | 1674 | GAGUAAACAACUUAAAAUUG | 1943 |
| 1736196 | csusa | | acsusc | | CUU | |
| AD- | asasaauaAfuUfUfAfcucacuc | 2215 | VPusCfsugaGfuGfAfguaaAfuUfauu | 1675 | CUAAAAUAAUUUACUCACUC | 1944 |
| 1736197 | asgsa | | uusasg | | AGA | |
| AD- | gsascaucUfaUfUfCfuguuggu | 2216 | VPusAfsgacCfaAfCfagaaUfaGfaug | 1676 | CUGACAUCUAUUCUGUUGGU | 1945 |
| 1736198 | csusa | | ucsasg | | CUG | |
| AD- | usasacugAfgUfAfGfucuuaua | 2217 | VPusAfsauaUfaAfGfacuaCfuCfagu | 1677 | CCUAACUGAGUAGUCUUAUA | 1946 |
| 1736199 | ususa | | uasgsg | | UUU | |
| AD- | uscsuggaAfuUfUfAfgccuucu | 2218 | VPusUfsuagAfaGfGfcuaaAfuUfcca | 1678 | CGUCUGGAAUUUAGCCUUCU | 1947 |
| 1736200 | asasa | | gascsg | | AAU | |
| AD- | ascsaugcCfuUfGfAfacucuug | 2219 | VPusUfsucaAfgAfGfuucaAfgGfcau | 1679 | AAACAUGCCUUGAACUCUUG | 1948 |
| 1736201 | asasa | | gususu | | AAC | |
| AD- | asuscaagCfaCfUfUfcuugcac | 2220 | VPusAfsaguGfcAfAfgaagUfgCfuug | 1680 | AAAUCAAGCACUUCUUGCAC | 1949 |
| 1736202 | ususa | | aususu | | UUG | |
| AD- | csusugcuUfuUfCfAfucuuuau | 2221 | VPusGfsuauAfaAfGfaugaAfaAfgca | 1681 | CACUUGCUUUUCAUCUUUAU | 1950 |
| 1736203 | ascsa | | agsusg | | ACU | |
| AD- | ususuucuCfcCfUfAfuguaaua | 2222 | VPusGfsauaUfuAfCfauagGfgAfgaa | 1682 | GCUUUUCUCCCUAUGUAAUA | 1951 |
| 1736204 | uscsa | | aasgsc | | UCU | |
| AD- | usgscuuuCfuUfCfUfaugucua | 2223 | VPusUfsuuaGfaCfAfuagaAfgAfaag | 1683 | UAUGCUUUCUUCUAUGUCUA | 1952 |
| 1736205 | asasa | | casusa | | AAA | |
| AD- | ascscugcUfuAfGfCfaaaucgu | 2224 | VPusAfsgacGfaUfUfugcuAfaGfcag | 1684 | GAACCUGCUUAGCAAAUCGU | 1953 |
| 1736206 | csusa | | gususc | | CUG | |
| AD- | asusauucCfuCfUfAfcccuuuc | 2225 | VPusGfsugaAfaGfGfguagAfgGfaau | 1685 | AGAUAUUCCUCUACCCUUUC | 1954 |
| 1736207 | ascsa | | auscsu | | ACA | |
| AD- | uscsccacUfuUfUfCfuuugggu | 2226 | VPusAfsaacCfcAfAfagaaAfaGfugg | 1686 | AAUCCCACUUUUCUUUGGGU | 1955 |
| 1736208 | ususa | | gasusu | | UUC | |
| AD- | asgsgcuuCfuUfGfUfauauacu | 2227 | VPusUfsaagUfaUfAfuacaAfgAfagc | 1687 | GCAGGCUUCUUGUAUAUACU | 1956 |
| 1736209 | usasa | | cusgsc | | UAU | |
| AD- | asasacuuGfaUfCfUfguguuuc | 2228 | VPusCfsugaAfaCfAfcagaUfcAfagu | 1688 | UCAAACUUGAUCUGUGUUUC | 1957 |
| 1736210 | asgsa | | uusgsa | | AGG | |
| AD- | ususugcuUfuAfAfUfgcuaaau | 2229 | VPusGfsaauUfuAfGfcauuAfaAfgca | 1689 | CUUUUGCUUUAAUGCUAAAU | 1958 |
| 1736211 | uscsa | | aasasg | | UCC | |
| AD- | asgsacuaGfcUfGfUfuauuugu | 2230 | VPusAfsuacAfaAfUfaacaGfcUfagu | 1690 | AAAGACUAGCUGUUAUUUGU | 1959 |
| 1736212 | asusa | | cususu | | AUU | |
| AD- | ususccuaCfuUfUfCfccuugaa | 2231 | VPusUfsauuCfaAfGfggaaAfgUfagg | 1691 | GCUUCCUACUUUCCCUUGAA | 1960 |
| 1736213 | usasa | | aasgsc | | UAG | |
| AD- | csasgaacUfgAfAfUfgcucaag | 2232 | VPusGfsacuUfgAfGfcauuCfaGfuuc | 1692 | UCCAGAACUGAAUGCUCAAG | 1961 |
| 1736214 | uscsa | | ugsgsa | | UCU | |
| AD- | uscsuuguUfaCfUfAfauuucuc | 2233 | VPusAfsugaGfaAfAfuuagUfaAfcaa | 1693 | GUUCUUGUUACUAAUUUCUC | 1962 |
| 1736215 | asusa | | gasasc | | AUA | |
| AD- | csuscaguGfaUfAfGfccuuuau | 2234 | VPusGfsuauAfaAfGfgcuaUfcAfcug | 1694 | AACUCAGUGAUAGCCUUUAU | 1963 |
| 1736216 | ascsa | | agsusu | | ACC | |
| AD- | ususuuauGfcUfCfCfuaaaaca | 2235 | VPusGfsaugUfuUfUfaggaGfcAfuaa | 1695 | UCUUUUAUGCUCCUAAAACA | 1964 |
| 1736217 | uscsa | | aasgsa | | UCU | |
| AD- | ususcuccUfuAfGfGfuuauguu | 2236 | VPusUfsgaaCfaUfAfaccuAfaGfgag | 1696 | AAUUCUCCUUAGGUUAUGUU | 1965 |
| 1736218 | csasa | | aasusu | | CAG | |
| AD- | asusugcuCfaUfCfGfagacaua | 2237 | VPusUfsuuaUfgUfCfucgaUfgAfgca | 1697 | CUAUUGCUCAUCGAGACAUA | 1966 |
| 1736219 | asasa | | ausasg | | AAA | |
| AD- | cscsuuugUfaCfAfUfacacacu | 2238 | VPusUfsaagUfgUfGfuaugUfaCfaaa | 1698 | UUCCUUUGUACAUACACACU | 1967 |
| 1736220 | usasa | | ggsasa | | UAG | |
| AD- | ususgaacCfaAfGfAfgcacaug | 2239 | VPusUfsucaUfgUfGfcucuUfgGfuuc | 1699 | ACUUGAACCAAGAGCACAUG | 1968 |
| 1736221 | asasa | | aasgsu | | AAU | |
| AD- | uscsagggAfuUfUfUfaaagucu | 2240 | VPusUfsuagAfcUfUfuaaaAfuCfccu | 1700 | CCUCAGGGAUUUUAAAGUCU | 1969 |
| 1736222 | asasa | | gasgsg | | AAU | |
| AD- | usascuugCfuUfAfUfaaaguga | 2241 | VPusUfsaucAfcUfUfuauaAfgCfaag | 1701 | GGUACUUGCUUAUAAAGUGA | 1970 |
| 1736223 | usasa | | uascsc | | UAG | |
| AD- | asusgcauCfuAfAfAfccuaccu | 2242 | VPusCfsaagGfuAfGfguuuAfgAfugc | 1702 | GCAUGCAUCUAAACCUACCU | 1971 |
| 1736224 | usgsa | | ausgsc | | UGA | |
| AD- | ususgggaAfaAfCfAfcuauuau | 2243 | VPusUfscauAfaUfAfguguUfuUfccc | 1703 | ACUUGGGAAAACACUAUUAU | 1972 |
| 1736225 | gsasa | | aasgsu | | GAA | |
| AD- | asasacucCfaAfAfGfagaaaug | 2244 | VPusUfsucaUfuUfCfucuuUfgGfagu | 1704 | UCAAACUCCAAAGAGAAAUG | 1973 |
| 1736226 | asasa | | uusgsa | | AAU | |
| AD- | asusgggcAfuUfUfUfucaaaac | 2245 | VPusAfsuguUfuUfGfaaaaAfuGfccc | 1705 | AUAUGGGCAUUUUUCAAAAC | 1974 |
| 1736227 | asusa | | ausasu | | AUU | |
| AD- | asusaaugGfaAfCfUfuggacuc | 2246 | VPusUfsugaGfuCfCfaaguUfcCfauu | 1706 | AGAUAAUGGAACUUGGACUC | 1975 |
| 1736228 | asasa | | auscsu | | AAC | |
| AD- | usgsuguaGfaUfGfAfcgaaacc | 2247 | VPusUfsuggUfuUfCfgucaUfcUfaca | 1707 | UUUGUGUAGAUGACGAAACC | 1976 |
| 1736229 | asasa | | casasa | | AAG | |
| AD- | ususcuacCfuCfAfAfagauaag | 2248 | VPusGfsucuUfaUfCfuuugAfgGfuag | 1708 | UGUUCUACCUCAAAGAUAAG | 1977 |
| 1736230 | ascsa | | aascsa | | ACA | |
| AD- | asgsaaggCfaUfAfUfugaguua | 2249 | VPusAfsauaAfcUfCfaauaUfgCfcuu | 1709 | UUAGAAGGCAUAUUGAGUUA | 1978 |
| 1736231 | ususa | | cusasa | | UUU | |
| AD- | asusggagUfuUfUfUfcugcuau | 2250 | VPusAfsaauAfgCfAfgaaaAfaCfucc | 1710 | ACAUGGAGUUUUUCUGCUAU | 1979 |
| 1736232 | ususa | | ausgsu | | UUU | |
| AD- | uscsaguuUfcUfCfAfuuaguuu | 2251 | VPusAfscaaAfcUfAfaugaGfaAfacu | 1711 | CCUCAGUUUCUCAUUAGUUU | 1980 |
| 1736233 | gsusa | | gasgsg | | GUC | |
| AD- | csusgcauAfuAfUfAfagaacga | 2252 | VPusUfsuucGfuUfCfuuauAfuAfugc | 1712 | UUCUGCAUAUAUAAGAACGA | 1981 |
| 1736234 | asasa | | agsasa | | AAU | |
| AD- | usgsagccAfaAfAfUfauuaaau | 2253 | VPusGfsaauUfuAfAfuauuUfuGfgcu | 1713 | UCUGAGCCAAAAUAUUAAAU | 1982 |
| 1736235 | uscsa | | casgsa | | UCU | |
| AD- | asgsaaugAfuAfUfUfuccuguu | 2254 | VPusAfsaaaCfaGfGfaaauAfuCfauu | 1714 | CAAGAAUGAUAUUUCCUGUU | 1983 |
| 1736236 | ususa | | cususg | | UUA | |
| AD- | gsusgcucUfaUfAfUfaaaucuu | 2255 | VPusGfsgaaGfaUfUfuauaUfaGfagc | 1715 | UUGUGCUCUAUAUAAAUCUU | 1984 |
| 1736237 | cscsa | | acsasa | | CCC | |
| AD- | ususuacaGfaGfAfAfgcaucuu | 2256 | VPusAfsuaaGfaUfGfcuucUfcUfgua | 1716 | AUUUUACAGAGAAGCAUCUU | 1985 |
| 1736238 | asusa | | aasasu | | AUU | |
| AD- | gsuscuugAfgUfUfCfugcauga | 2257 | VPusUfsgucAfuGfCfagaaCfuCfaag | 1717 | AUGUCUUGAGUUCUGCAUGA | 1986 |
| 1736239 | csasa | | acsasu | | CAG | |
| AD- | csusucaaAfuCfUfAfuaauuuu | 2258 | VPusGfsuaaAfaUfUfauagAfuUfuga | 1718 | CCCUUCAAAUCUAUAAUUUU | 1987 |
| 1736240 | ascsa | | agsgsg | | ACU | |
| AD- | gsasagcaUfaUfAfGfaacucua | 2259 | VPusAfsauaGfaGfUfucuaUfaUfgcu | 1719 | GUGAAGCAUAUAGAACUCUA | 1988 |
| 1736241 | ususa | | ucsasc | | UUU | |
| AD- | gsasgcugCfuUfCfUfaaauaac | 2260 | VPusUfsuguUfaUfUfuagaAfgCfagc | 1720 | GAGAGCUGCUUCUAAAUAAC | 1989 |
| 1736242 | asasa | | ucsusc | | AAA | |
| AD- | usgsacugUfaUfUfUfccugauc | 2261 | VPusAfsugaUfcAfGfgaaaUfaCfagu | 1721 | UGUGACUGUAUUUCCUGAUC | 1990 |
| 1736243 | asusa | | cascsa | | AUU | |
| AD- | gsusucugAfaGfGfAfuauacuu | 2262 | VPusAfsgaaGfuAfUfauccUfuCfaga | 1722 | GCGUUCUGAAGGAUAUACUU | 1991 |
| 1736244 | csusa | | acsgsc | | CUG | |
| AD- | gsusgaagCfaUfGfAfuucaaua | 2263 | VPusAfsguaUfuGfAfaucaUfgCfuuc | 1723 | CUGUGAAGCAUGAUUCAAUA | 1992 |
| 1736245 | csusa | | acsasg | | CUG | |
| AD- | asasugcuGfcUfUfCfacagauu | 2264 | VPusAfsaaaUfcUfGfugaaGfcAfgca | 1724 | CGAAUGCUGCUUCACAGAUU | 1993 |
| 1736246 | ususa | | uuscsg | | UUU | |
| AD- | usgsugggUfuAfUfGfuuaaucu | 2265 | VPusUfscagAfuUfAfacauAfaCfcca | 1725 | ACUGUGGGUUAUGUUAAUCU | 1994 |
| 1736247 | gsasa | | casgsu | | GAA | |
| AD- | gsasguacAfgAfAfGfaaugcuc | 2266 | VPusAfsugaGfcAfUfucuuCfuGfuac | 1726 | GUGAGUACAGAAGAAUGCUC | 1995 |
| 1736248 | asusa | | ucsasc | | AUG | |
| AD- | csusguauUfuCfAfUfccuagau | 2267 | VPusAfsaauCfuAfGfgaugAfaAfuac | 1727 | AGCUGUAUUUCAUCCUAGAU | 1996 |
| 1736249 | ususa | | agscsu | | UUU | |
| AD- | asasucugGfuAfAfAfugcugga | 2268 | VPusUfsuucCfaGfCfauuuAfcCfaga | 1728 | GCAAUCUGGUAAAUGCUGGA | 1997 |
| 1736250 | asasa | | uusgsc | | AAA | |
| AD- | usgscucaGfaUfUfAfccugauc | 2269 | VPusAfscgaUfcAfGfguaaUfcUfgag | 1729 | AUUGCUCAGAUUACCUGAUC | 1998 |
| 1736251 | gsusa | | casasu | | GUG | |
| AD- | gsascagcUfaUfGfGfaguuugc | 2270 | VPusAfscgcAfaAfCfuccaUfaGfcug | 1730 | CUGACAGCUAUGGAGUUUGC | 1999 |
| 1736252 | gsusa | | ucsasg | | GUG | |
| AD- | usasgaacAfuGfCfUfcacuuac | 2271 | VPusUfsuguAfaGfUfgagcAfuGfuuc | 1731 | UAUAGAACAUGCUCACUUAC | 2000 |
| 1736253 | asasa | | uasusa | | AAA | |
| AD- | asusuaugCfuUfCfAfuuucacu | 2272 | VPusAfsuagUfgAfAfaugaAfgCfaua | 1732 | UGAUUAUGCUUCAUUUCACU | 2001 |
| 1736254 | asusa | | auscsa | | AUG | |
| AD- | csasacagCfaAfGfUfcauaaaa | 2273 | VPusAfscuuUfuAfUfgacuUfgCfugu | 1733 | ACCAACAGCAAGUCAUAAAA | 2002 |
| 1736255 | gsusa | | ugsgsu | | GUA | |
| AD- | ususgucuGfaAfAfAfugucuuu | 2274 | VPusUfsuaaAfgAfCfauuuUfcAfgac | 1734 | CAUUGUCUGAAAAUGUCUUU | 2003 |
| 1736256 | asasa | | aasusg | | AAG | |
| AD- | usgsuuggUfuCfAfAfaggacaa | 2275 | VPusAfsauuGfuCfCfuuugAfaCfcaa | 1735 | UCUGUUGGUUCAAAGGACAA | 2004 |
| 1736257 | ususa | | casgsa | | UUG | |
| AD- | uscsuaaaCfuUfUfAfauauauc | 2276 | VPusUfscgaUfaUfAfuuaaAfgUfuua | 1736 | GCUCUAAACUUUAAUAUAUC | 2005 |
| 1736258 | gsasa | | gasgsc | | GAA | |
| AD- | asasgugaAfaCfAfUfgacuguc | 2277 | VPusAfscgaCfaGfUfcaugUfuUfcac | 1737 | UUAAGUGAAACAUGACUGUC | 2006 |
| 1736259 | gsusa | | uusasa | | GUG | |
| AD- | asuscgucCfaUfAfCfaguuuuc | 2278 | VPusAfsugaAfaAfCfuguaUfgGfacg | 1738 | AUAUCGUCCAUACAGUUUUC | 2007 |
| 1736260 | asusa | | ausasu | | AUU | |
| AD- | csusucagGfaAfAfUfaguagga | 2279 | VPusUfsuucCfuAfCfuauuUfcCfuga | 1739 | UGCUUCAGGAAAUAGUAGGA | 2008 |
| 1736261 | asasa | | agscsa | | AAA | |
| AD- | csascccuUfuCfUfGfuaaggac | 2280 | VPusCfsaguCfcUfUfacagAfaAfggg | 1740 | CCCACCCUUUCUGUAAGGAC | 2009 |
| 1736262 | usgsa | | ugsgsg | | UGU | |
| AD- | asasagagAfuAfCfCfugacauc | 2281 | VPusAfsggaUfgUfCfagguAfuCfucu | 1741 | CAAAAGAGAUACCUGACAUC | 2010 |
| 1736263 | csusa | | uususg | | CUG | |
| AD- | asusgagcAfuUfAfAfuguuuuc | 2282 | VPusCfsagaAfaAfCfauuaAfuGfcuc | 1742 | UAAUGAGCAUUAAUGUUUUC | 2011 |
| 1736264 | usgsa | | aususa | | UGA | |
| AD- | usgsacauUfuUfUfUfcucauag | 2283 | VPusUfsucuAfuGfAfgaaaAfaAfugu | 1743 | GUUGACAUUUUUUCUCAUAG | 2012 |
| 1736265 | asasa | | casasc | | AAA | |
| AD- | csasgagaUfuAfAfAfugacuac | 2284 | VPusUfsaguAfgUfCfauuuAfaUfcuc | 1744 | AUCAGAGAUUAAAUGACUAC | 2013 |
| 1736266 | usasa | | ugsasu | | UAG | |
| AD- | gscsugucAfaAfUfGfuuauauu | 2285 | VPusAfscaaUfaUfAfacauUfuGfaca | 1745 | AUGCUGUCAAAUGUUAUAUU | 2014 |
| 1736267 | gsusa | | gcsasu | | GUU | |
| AD- | asasuauaAfuAfCfUfacagcaa | 2286 | VPusUfsuuuGfcUfGfuaguAfuUfaua | 1746 | GGAAUAUAAUACUACAGCAA | 2015 |
| 1736268 | asasa | | uuscsc | | AAU | |
| AD- | gsgsugucUfaAfAfUfauaauuu | 2287 | VPusUfsgaaAfuUfAfuauuUfaGfaca | 1747 | AGGGUGUCUAAAUAUAAUUU | 2016 |
| 1736269 | csasa | | ccscsu | | CAU | |
| AD- | csuscuuuAfuAfCfAfaaaugag | 2288 | VPusCfsucuCfaUfUfuuguAfuAfaag | 1748 | UUCUCUUUAUACAAAAUGAG | 2017 |
| 1736270 | asgsa | | agsasa | | AGU | |
| AD- | gscsaguaCfcAfAfAfugaauaa | 2289 | VPusUfsuuuAfuUfCfauuuGfgUfacu | 1749 | UAGCAGUACCAAAUGAAUAA | 2018 |
| 1736271 | asasa | | gcsusa | | AAG | |
| AD- | ususguugGfuAfCfAfcaaggua | 2290 | VPusUfsuuaCfcUfUfguguAfcCfaac | 1750 | GAUUGUUGGUACACAAGGUA | 2019 |
| 1736272 | asasa | | aasusc | | AAC | |
| AD- | csuscuuaUfuUfAfAfcuuaacc | 2291 | VPusAfscggUfuAfAfguuaAfaUfaag | 1751 | AACUCUUAUUUAACUUAACC | 2020 |
| 1736273 | gsusa | | agsusu | | GUC | |
| AD- | usgscuugAfuGfAfUfacaauga | 2292 | VPusAfsuucAfuUfGfuaucAfuCfaag | 1752 | AAUGCUUGAUGAUACAAUGA | 2021 |
| 1736274 | asusa | | casusu | | AUG | |
| AD- | asascuggGfaAfAfGfacaauuu | 2293 | VPusUfscaaAfuUfGfucuuUfcCfcag | 1753 | GUAACUGGGAAAGACAAUUU | 2022 |
| 1736275 | gsasa | | uusasc | | GAA | |
| AD- | csusccuuAfuAfUfGfacuauuu | 2294 | VPusUfscaaAfuAfGfucauAfuAfagg | 1754 | GGCUCCUUAUAUGACUAUUU | 2023 |
| 1736276 | gsasa | | agscsc | | GAA | |
| AD- | asgsucauGfcUfAfAfccaaugg | 2295 | VPusUfsuccAfuUfGfguuaGfcAfuga | 1755 | UCAGUCAUGCUAACCAAUGG | 2024 |
| 1736277 | asasa | | cusgsa | | AAA | |
| AD- | cscsugaaGfuCfAfCfacagcua | 2296 | VPusAfsuuaGfcUfGfugugAfcUfuca | 1756 | GUCCUGAAGUCACACAGCUA | 2025 |
| 1736278 | asusa | | ggsasc | | AUG | |
| AD- | ususgcugCfuGfAfCfaacaaag | 2297 | VPusAfsucuUfuGfUfugucAfgCfagc | 1757 | CAUUGCUGCUGACAACAAAG | 2026 |
| 1736279 | asusa | | aasusg | | AUA | |
| AD- | usascugaCfuUfUfGfccuguaa | 2298 | VPusCfsuuuAfcAfGfgcaaAfgUfcag | 1758 | AUUACUGACUUUGCCUGUAA | 2027 |
| 1736280 | asgsa | | uasasu | | AGA | |
| AD- | asasucccUfaUfUfUfuaaaauu | 2299 | VPusGfsgaaUfuUfUfaaaaUfaGfgga | 1759 | ACAAUCCCUAUUUUAAAAUU | 2028 |
| 1736281 | cscsa | | uusgsu | | CCC | |
| AD- | gsusgccuUfaGfGfAfgauuacc | 2300 | VPusAfsgggUfaAfUfcuccUfaAfggc | 1760 | GAGUGCCUUAGGAGAUUACC | 2029 |
| 1736282 | csusa | | acsusc | | CUU | |
| AD- | gsascaauCfuAfAfAfuauauca | 2301 | VPusGfsaugAfuAfUfauuuAfgAfuug | 1761 | CAGACAAUCUAAAUAUAUCA | 2030 |
| 1736283 | uscsa | | ucsusg | | UCA | |
| AD- | usgsugauAfaUfGfAfacaccgu | 2302 | VPusUfsuacGfgUfGfuucaUfuAfuca | 1762 | CUUGUGAUAAUGAACACCGU | 2031 |
| 1736284 | asasa | | casasg | | AAG | |
| AD- | uscsaccgUfuGfUfAfaagacuu | 2303 | VPusAfsaaaGfuCfUfuuacAfaCfggu | 1763 | AAUCACCGUUGUAAAGACUU | 2032 |
| 1736285 | ususa | | gasusu | | UUU | |
| AD- | ususagagAfaGfCfUfuccugac | 2304 | VPusAfsuguCfaGfGfaagcUfuCfucu | 1764 | GAUUAGAGAAGCUUCCUGAC | 2033 |
| 1736286 | asusa | | aasusc | | AUC | |
| AD- | asascaguAfaAfGfAfaaugcua | 2305 | VPusAfsguaGfcAfUfuucuUfuAfcug | 1765 | AUAACAGUAAAGAAAUGCUA | 2034 |
| 1736287 | csusa | | uusasu | | CUU | |
| AD- | usasuguuCfuGfAfCfuugagag | 2306 | VPusAfsacuCfuCfAfagucAfgAfaca | 1766 | UUUAUGUUCUGACUUGAGAG | 2035 |
| 1736288 | ususa | | uasasa | | UUA | |
| AD- | ususagagCfaUfGfCfuaucuuu | 2307 | VPusCfsuaaAfgAfUfagcaUfgCfucu | 1767 | AAUUAGAGCAUGCUAUCUUU | 2036 |
| 1736289 | asgsa | | aasusu | | AGG | |
| AD- | usasgucuUfuGfAfUfugaaaua | 2308 | VPusCfsuuaUfuUfCfaaucAfaAfgac | 1768 | CAUAGUCUUUGAUUGAAAUA | 2037 |
| 1736290 | asgsa | | uasusg | | AGU | |
| AD- | ususgguaAfaCfGfAfgauuuaa | 2309 | VPusUfsguuAfaAfUfcucgUfuUfacc | 1769 | CUUUGGUAAACGAGAUUUAA | 2038 |
| 1736291 | csasa | | aasasg | | CAU | |
| AD- | gsuscugaAfaAfUfUfgcuuuca | 2310 | VPusAfsaugAfaAfGfcaauUfuUfcag | 1770 | AGGUCUGAAAAUUGCUUUCA | 2039 |
| 1736292 | ususa | | acscsu | | UUU | |
| AD- | asusgaacUfuGfUfUfgccuugu | 2311 | VPusUfsuacAfaGfGfcaacAfaGfuuc | 1771 | GCAUGAACUUGUUGCCUUGU | 2040 |
| 1736293 | asasa | | ausgsc | | AAA | |
| AD- | gsgsgcaaUfaAfAfCfuguauca | 2312 | VPusUfsuugAfuAfCfaguuUfaUfugc | 1772 | UUGGGCAAUAAACUGUAUCA | 2041 |
| 1736294 | asasa | | ccsasa | | AAA | |
| AD- | ascsauguUfaGfCfAfuauaaug | 2313 | VPusUfsacaUfuAfUfaugcUfaAfcau | 1773 | GUACAUGUUAGCAUAUAAUG | 2042 |
| 1736295 | usasa | | gusasc | | UAU | |
| AD- | usgsugagAfgUfAfUfagaauuu | 2314 | VPusAfsgaaAfuUfCfuauaCfuCfuca | 1774 | ACUGUGAGAGUAUAGAAUUU | 2043 |
| 1736296 | csusa | | casgsu | | CUU | |
| AD- | asusccuuCfaUfUfUfggcacua | 2315 | VPusUfsauaGfuGfCfcaaaUfgAfagg | 1775 | UAAUCCUUCAUUUGGCACUA | 2044 |
| 1736297 | usasa | | aususa | | UAG | |
| AD- | ascsauuuAfgAfAfAfguagcuu | 2316 | VPusUfsaaaGfcUfAfcuuuCfuAfaau | 1776 | ACACAUUUAGAAAGUAGCUU | 2045 |
| 1736298 | usasa | | gusgsu | | UAU | |
| AD- | asascuguUfcUfAfUfaaagcaa | 2317 | VPusCfsuuuGfcUfUfuauaGfaAfcag | 1777 | UUAACUGUUCUAUAAAGCAA | 2046 |
| 1736299 | asgsa | | uusasa | | AGC | |
| AD- | cscsuccaGfaGfAfUfgaaagau | 2318 | VPusAfsgauCfuUfUfcaucUfcUfgga | 1778 | CUCCUCCAGAGAUGAAAGAU | 2047 |
| 1736300 | csusa | | ggsasg | | CUU | |
| AD- | ususguuuAfaCfUfAfugacucc | 2319 | VPusUfsaggAfgUfCfauagUfuAfaac | 1779 | UGUUGUUUAACUAUGACUCC | 2048 |
| 1736301 | usasa | | aascsa | | UAA | |
| AD- | csusguuuGfaCfAfAfugcuuug | 2320 | VPusAfsacaAfaGfCfauugUfcAfaac | 1780 | UUCUGUUUGACAAUGCUUUG | 2049 |
| 1736302 | ususa | | agsasa | | UUC | |
| AD- | usgsuccuAfaAfAfGfaaauuuu | 2321 | VPusGfsaaaAfaUfUfucuuUfuAfgga | 1781 | GUUGUCCUAAAAGAAAUUUU | 2050 |
| 1736303 | uscsa | | casasc | | UCU | |
| AD- | ascsagauUfgAfAfCfaaagaac | 2322 | VPusAfsaguUfcUfUfuguuCfaAfucu | 1782 | CUACAGAUUGAACAAAGAAC | 2051 |
| 1736304 | ususa | | gusasg | | UUA | |
| AD- | gsusuuaaCfcUfGfUfccaaacu | 2323 | VPusGfsaagUfuUfGfgacaGfgUfuaa | 1783 | UCGUUUAACCUGUCCAAACU | 2052 |
| 1736305 | uscsa | | acsgsa | | UCU | |
| AD- | csasaaauGfuUfUfAfacuuuac | 2324 | VPusUfsgguAfaAfGfuuaaAfcAfuuu | 1784 | ACCAAAAUGUUUAACUUUAC | 2053 |
| 1736306 | csasa | | ugsgsu | | CAA | |
| AD- | uscsccauUfgUfGfUfaauauuu | 2325 | VPusAfsuaaAfuAfUfuacaCfaAfugg | 1785 | AGUCCCAUUGUGUAAUAUUU | 2054 |
| 1736307 | asusa | | gascsu | | AUU | |
| AD- | gsusugggUfaAfAfUfaugcuua | 2326 | VPusAfsauaAfgCfAfuauuUfaCfcca | 1786 | AUGUUGGGUAAAUAUGCUUA | 2055 |
| 1736308 | ususa | | acsasu | | UUG | |
| AD- | csasggguUfgUfCfUfuugaguc | 2327 | VPusCfsagaCfuCfAfaagaCfaAfccc | 1787 | CUCAGGGUUGUCUUUGAGUC | 2056 |
| 1736309 | usgsa | | ugsasg | | UGC | |
| AD- | gscsuaaaAfuCfCfAfacugcaa | 2328 | VPusAfsauuGfcAfGfuuggAfuUfuua | 1788 | GGGCUAAAAUCCAACUGCAA | 2057 |
| 1736310 | ususa | | gcscsc | | UUG | |
| AD- | csasggacUfgAfAfGfuguguau | 2329 | VPusAfscauAfcAfCfacuuCfaGfucc | 1789 | GCCAGGACUGAAGUGUGUAU | 2058 |
| 1736311 | gsusa | | ugsgsc | | GUC | |
| AD- | gsasguacCfaAfUfUfgccuuau | 2330 | VPusUfsaauAfaGfGfcaauUfgGfuac | 1790 | AGGAGUACCAAUUGCCUUAU | 2059 |
| 1736312 | usasa | | ucscsu | | UAU | |
| AD- | asasgugcCfuAfUfGfugaagug | 2331 | VPusAfsucaCfuUfCfacauAfgGfcac | 1791 | AAAAGUGCCUAUGUGAAGUG | 2060 |
| 1736313 | asusa | | uususu | | AUU | |
| AD- | usasacaaUfaAfUfAfgaacugc | 2332 | VPusUfsggcAfgUfUfcuauUfaUfugu | 1792 | CCUAACAAUAAUAGAACUGC | 2061 |
| 1736314 | csasa | | uasgsg | | CAG | |
| AD- | gscsuguuCfcAfUfAfccauugc | 2333 | VPusAfsagcAfaUfGfguauGfgAfaca | 1793 | CUGCUGUUCCAUACCAUUGC | 2062 |
| 1736315 | ususa | | gcsasg | | UUU | |
| AD- | usasgacuGfgAfGfAfagauuau | 2334 | VPusGfsaauAfaUfCfuucuCfcAfguc | 1794 | UCUAGACUGGAGAAGAUUAU | 2063 |
| 1736316 | uscsa | | uasgsa | | UCA | |
| AD- | ususccaaCfaGfCfAfucaccaa | 2335 | VPusAfsuuuGfgUfGfaugcUfgUfugg | 1795 | CCUUCCAACAGCAUCACCAA | 2064 |
| 1736317 | asusa | | aasgsg | | AUG | |
| AD- | usasauugGfuAfCfAfgauucug | 2336 | VPusAfsacaGfaAfUfcuguAfcCfaau | 1796 | UGUAAUUGGUACAGAUUCUG | 2065 |
| 1736318 | ususa | | uascsa | | UUG | |
| AD- | gsasgaaaCfaUfUfAfuuuguug | 2337 | VPusGfsacaAfcAfAfauaaUfgUfuuc | 1797 | AAGAGAAACAUUAUUUGUUG | 2066 |
| 1736319 | uscsa | | ucsusu | | UCA | |
| AD- | csgscucuCfaAfUfUfgcuagug | 2338 | VPusAfsccaCfuAfGfcaauUfgAfgag | 1798 | GGCGCUCUCAAUUGCUAGUG | 2067 |
| 1736320 | gsusa | | cgscsc | | GUC | |
| AD- | csgsguauCfaGfAfAfacagcaa | 2339 | VPusAfsuuuGfcUfGfuuucUfgAfuac | 1799 | GACGGUAUCAGAAACAGCAA | 2068 |
| 1736321 | asusa | | cgsusc | | AUA | |
| AD- | cscscagaCfaUfAfAfuuuuaua | 2340 | VPusAfsauaUfaAfAfauuaUfgUfcug | 1800 | CUCCCAGACAUAAUUUUAUA | 2069 |
| 1736322 | ususa | | ggsasg | | UUU | |
| AD- | usasguacAfuUfUfUfgagguau | 2341 | VPusAfsaauAfcCfUfcaaaAfuGfuac | 1801 | GUUAGUACAUUUUGAGGUAU | 2070 |
| 1736323 | ususa | | uasasc | | UUU | |
| AD- | asasaugcUfaUfUfGfauaacag | 2342 | VPusUfsacuGfuUfAfucaaUfaGfcau | 1802 | GUAAAUGCUAUUGAUAACAG | 2071 |
| 1736324 | usasa | | uusasc | | UAA | |
| AD- | usasgcugAfaUfUfUfgggacua | 2343 | VPusCfsauaGfuCfCfcaaaUfuCfagc | 1803 | GAUAGCUGAAUUUGGGACUA | 2072 |
| 1736325 | usgsa | | uasusc | | UGU | |
| AD- | asusugugGfuUfUfUfcaaagau | 2344 | VPusAfsuauCfuUfUfgaaaAfcCfaca | 1804 | CCAUUGUGGUUUUCAAAGAU | 2073 |
| 1736326 | asusa | | ausgsg | | AUU | |
| AD- | cscsugguUfgCfAfGfuaucuga | 2345 | VPusUfsuucAfgAfUfacugCfaAfcca | 1805 | AACCUGGUUGCAGUAUCUGA | 2074 |
| 1736327 | asasa | | ggsusu | | AAA | |
| AD- | usasgaauGfgUfUfGfuugagcu | 2346 | VPusUfsuagCfuCfAfacaaCfcAfuuc | 1806 | UGUAGAAUGGUUGUUGAGCU | 2075 |
| 1736328 | asasa | | uascsa | | AAG | |
| AD- | csusggccAfuCfAfUfuauuacu | 2347 | VPusAfscagUfaAfUfaaugAfuGfgcc | 1807 | AGCUGGCCAUCAUUAUUACU | 2076 |
| 1736329 | gsusa | | agscsu | | GUG | |
| AD- | ususgacaGfaUfAfAfaauacga | 2348 | VPusCfsuucGfuAfUfuuuaUfcUfguc | 1808 | AAUUGACAGAUAAAAUACGA | 2077 |
| 1736330 | asgsa | | aasusu | | AGU | |
| AD- | uscsacaaAfcAfCfAfucauuac | 2349 | VPusUfsuguAfaUfGfauguGfuUfugu | 1809 | AUUCACAAACACAUCAUUAC | 2078 |
| 1736331 | asasa | | gasasu | | AAG | |
| AD- | cscsaugaUfuGfUfAfugaaaau | 2350 | VPusCfsuauUfuUfCfauacAfaUfcau | 1810 | UUCCAUGAUUGUAUGAAAAU | 2079 |
| 1736332 | asgsa | | ggsasa | | AGU | |
| AD- | csascagaGfuAfAfUfgaauauu | 2351 | VPusUfsaaaUfaUfUfcauuAfcUfcug | 1811 | GACACAGAGUAAUGAAUAUU | 2080 |
| 1736333 | usasa | | ugsusc | | UAA | |
| AD- | asgscugcUfuAfGfUfggaagau | 2352 | VPusAfscauCfuUfCfcacuAfaGfcag | 1812 | GGAGCUGCUUAGUGGAAGAU | 2081 |
| 1736334 | gsusa | | cuscsc | | GUA | |
| AD- | usasgcucUfgUfGfUfgcugaua | 2353 | VPusGfsguaUfcAfGfcacaCfaGfagc | 1813 | UCUAGCUCUGUGUGCUGAUA | 2082 |
| 1736335 | cscsa | | uasgsa | | CCU | |
| AD- | csasuucgAfgAfGfAfauaugag | 2354 | VPusAfsgcuCfaUfAfuucuCfuCfgaa | 1814 | UACAUUCGAGAGAAUAUGAG | 2083 |
| 1736336 | csusa | | ugsusa | | CUG | |
| AD- | asusgcauUfuUfCfUfgaaaaug | 2355 | VPusUfsacaUfuUfUfcagaAfaAfugc | 1815 | GUAUGCAUUUUCUGAAAAUG | 2084 |
| 1736337 | usasa | | ausasc | | UAU | |
| AD- | gsusuucgAfcCfAfAfguauccc | 2356 | VPusUfsuggGfaUfAfcuugGfuCfgaa | 1816 | AAGUUUCGACCAAGUAUCCC | 2085 |
| 1736338 | asasa | | acsusu | | AAA | |
| AD- | uscsuugcUfuGfCfCfacaauau | 2357 | VPusCfsgauAfuUfGfuggcAfaGfcaa | 1817 | UAUCUUGCUUGCCACAAUAU | 2086 |
| 1736339 | csgsa | | gasusa | | CGG | |
| AD- | gsgsguacAfaGfAfGfggaauac | 2358 | VPusUfsuguAfuUfCfccucUfuGfuac | 1818 | UCGGGUACAAGAGGGAAUAC | 2087 |
| 1736340 | asasa | | ccsgsa | | AAA | |
| AD- | gsgsugauCfaAfAfUfccugugu | 2359 | VPusAfsgacAfcAfGfgauuUfgAfuca | 1819 | CAGGUGAUCAAAUCCUGUGU | 2088 |
| 1736341 | csusa | | ccsusg | | CUC | |
| AD- | csasguggGfuUfUfCfuaggaua | 2360 | VPusCfscuaUfcCfUfagaaAfcCfcac | 1820 | GACAGUGGGUUUCUAGGAUA | 2089 |
| 1736342 | gsgsa | | ugsusc | | GGU | |
| AD- | asascuaaGfuAfUfUfuuuaagg | 2361 | VPusGfsuccUfuAfAfaaauAfcUfuag | 1821 | GUAACUAAGUAUUUUUAAGG | 2090 |
| 1736343 | ascsa | | uusasc | | ACA | |
| AD- | csasuaauGfuGfAfUfuguuaaa | 2362 | VPusAfsauuUfaAfCfaaucAfcAfuua | 1822 | CCCAUAAUGUGAUUGUUAAA | 2091 |
| 1736344 | ususa | | ugsgsg | | UUU | |
| AD- | gsasgaagAfaUfUfAfagaaaac | 2363 | VPusGfsaguUfuUfCfuuaaUfuCfuuc | 1823 | UUGAGAAGAAUUAAGAAAAC | 2092 |
| 1736345 | uscsa | | ucsasa | | UCU | |
| AD- | gsasagagAfaAfUfUfgauuguu | 2364 | VPusUfsaaaCfaAfUfcaauUfuCfucu | 1824 | GAGAAGAGAAAUUGAUUGUU | 2093 |
| 1736346 | usasa | | ucsusc | | UAU | |
| AD- | asgsugugAfaAfCfUfugugcca | 2365 | VPusUfsaugGfcAfCfaaguUfuCfaca | 1825 | AAAGUGUGAAACUUGUGCCA | 2094 |
| 1736347 | usasa | | cususu | | UAG | |
| AD- | csusgagaCfaCfUfGfagaaaug | 2366 | VPusGfsacaUfuUfCfucagUfgUfcuc | 1826 | UCCUGAGACACUGAGAAAUG | 2095 |
| 1736348 | uscsa | | agsgsa | | UCC | |
| AD- | usgsgcuuAfuAfUfGfuguuuaa | 2367 | VPusCfsuuuAfaAfCfacauAfuAfagc | 1827 | AUUGGCUUAUAUGUGUUUAA | 2096 |
| 1736349 | asgsa | | casasu | | AGA | |
| AD- | usgscauuUfuGfAfCfauugcaa | 2368 | VPusUfsuuuGfcAfAfugucAfaAfaug | 1828 | AUUGCAUUUUGACAUUGCAA | 2097 |
| 1736350 | asasa | | casasu | | AAC | |
| AD- | ususagguUfaGfAfAfguuccag | 2369 | VPusUfsucuGfgAfAfcuucUfaAfccu | 1829 | CAUUAGGUUAGAAGUUCCAG | 2098 |
| 1736351 | asasa | | aasusg | | AAU | |
| AD- | usgscgacAfuGfAfAfaacaucc | 2370 | VPusAfsaggAfuGfUfuuucAfuGfucg | 1830 | GCUGCGACAUGAAAACAUCC | 2099 |
| 1736352 | ususa | | casgsc | | UUG | |
| AD- | gsasgaggAfuUfUfUfuuaccau | 2371 | VPusAfsgauGfgUfAfaaaaAfuCfcuc | 1831 | AAGAGAGGAUUUUUUACCAU | 2100 |
| 1736353 | csusa | | ucsusu | | CUC | |
| AD- | gsasagguGfaAfGfAfgaauuuc | 2372 | VPusUfsugaAfaUfUfcucuUfcAfccu | 1832 | UGGAAGGUGAAGAGAAUUUC | 2101 |
| 1736354 | asasa | | ucscsa | | AAA | |
| AD- | usgsguauCfuGfAfAfuaucaug | 2373 | VPusUfsucaUfgAfUfauucAfgAfuac | 1833 | GCUGGUAUCUGAAUAUCAUG | 2102 |
| 1736355 | asasa | | casgsc | | AAC | |
| AD- | usgsuuggUfuUfAfAfuuuuuca | 2374 | VPusGfsuugAfaAfAfauuaAfaCfcaa | 1834 | CAUGUUGGUUUAAUUUUUCA | 2103 |
| 1736356 | ascsa | | casusg | | ACC | |
| AD- | usasguugUfaAfUfUfuaaaugu | 2375 | VPusCfscacAfuUfUfaaauUfaCfaac | 1835 | AGUAGUUGUAAUUUAAAUGU | 2104 |
| 1736357 | gsgsa | | uascsu | | GGA | |
| AD- | gsusgaauGfuUfUfUfugccauu | 2376 | VPusAfsaaaUfgGfCfaaaaAfcAfuuc | 1836 | AUGUGAAUGUUUUUGCCAUU | 2105 |
| 1736358 | ususa | | acsasu | | UUU | |
| AD- | csasgagaUfgAfUfUfuuucuuu | 2377 | VPusUfsaaaAfgAfAfaaauCfaUfcuc | 1837 | GGCAGAGAUGAUUUUUCUUU | 2106 |
| 1736359 | usasa | | ugscsc | | UAA | |
| AD- | asgsaaaaUfuAfGfAfuucagau | 2378 | VPusAfsgauCfuGfAfaucuAfaUfuuu | 1838 | ACAGAAAAUUAGAUUCAGAU | 2107 |
| 1736360 | csusa | | cusgsu | | CUC | |
| AD- | ascsuggaAfaAfUfUfaagaaag | 2379 | VPusUfsacuUfuCfUfuaauUfuUfcca | 1839 | CCACUGGAAAAUUAAGAAAG | 2108 |
| 1736361 | usasa | | gusgsg | | UAG | |
| AD- | usasaaguGfgGfAfAfagauaau | 2380 | VPusUfsuauUfaUfCfuuucCfcAfcuu | 1840 | UGUAAAGUGGGAAAGAUAAU | 2109 |
| 1736362 | asasa | | uascsa | | AAA | |
| AD- | csusaggcUfaAfGfAfaagaguu | 2381 | VPusAfscaaCfuCfUfuucuUfaGfccu | 1841 | AACUAGGCUAAGAAAGAGUU | 2110 |
| 1736363 | gsusa | | agsusu | | GUA | |
| AD- | gscsugauAfaAfAfUfuaaugga | 2382 | VPusUfsaucCfaUfUfaauuUfuAfuca | 1842 | GUGCUGAUAAAAUUAAUGGA | 2111 |
| 1736364 | usasa | | gcsasc | | UAA | |
| AD- | cscsuguaUfuUfCfUfuuaguug | 2383 | VPusGfsacaAfcUfAfaagaAfaUfaca | 1843 | UUCCUGUAUUUCUUUAGUUG | 2112 |
| 1736365 | uscsa | | ggsasa | | UCG | |
| AD- | ususuguuUfuGfCfUfuuugaca | 2384 | VPusUfsuugUfcAfAfaagcAfaAfaca | 1844 | GCUUUGUUUUGCUUUUGACA | 2113 |
| 1736366 | asasa | | aasgsc | | AAC | |
| AD- | usgsgcugUfgAfAfAfauauucu | 2385 | VPusGfsgagAfaUfAfuuuuCfaCfagc | 1845 | UGUGGCUGUGAAAAUAUUCU | 2114 |
| 1736367 | cscsa | | cascsa | | CCU | |
| AD- | ascsuauuUfuAfGfGfcauagca | 2386 | VPusAfsgugCfuAfUfgccuAfaAfaua | 1846 | GCACUAUUUUAGGCAUAGCA | 2115 |
| 1736368 | csusa | | gusgsc | | CUU | |
| AD- | ususucagUfaCfUfGfuauaaag | 2387 | VPusCfsacuUfuAfUfacagUfaCfuga | 1847 | AUUUUCAGUACUGUAUAAAG | 2116 |
| 1736369 | usgsa | | aasasu | | UGG | |
| AD- | gsusgaugCfaAfUfCfuauguuu | 2388 | VPusGfsgaaAfcAfUfagauUfgCfauc | 1848 | AGGUGAUGCAAUCUAUGUUU | 2117 |
| 1736370 | cscsa | | acscsu | | CCC | |
| AD- | usgsuuugGfcAfGfAfucucauc | 2389 | VPusUfsugaUfgAfGfaucuGfcCfaaa | 1849 | AAUGUUUGGCAGAUCUCAUC | 2118 |
| 1736371 | asasa | | casusu | | AAU | |
| AD- | asasgguuGfuUfUfGfugaccag | 2390 | VPusUfsucuGfgUfCfacaaAfcAfacc | 1850 | GAAAGGUUGUUUGUGACCAG | 2119 |
| 1736372 | asasa | | uususc | | AAG | |
| AD- | gsgsaaggAfaAfUfAfuuuugag | 2391 | VPusUfsucuCfaAfAfauauUfuCfcuu | 1851 | AAGGAAGGAAAUAUUUUGAG | 2120 |
| 1736373 | asasa | | ccsusu | | AAU | |
| AD- | ususuuggUfaGfCfGfugacucu | 2392 | VPusGfsaagAfgUfCfacgcUfaCfcaa | 1852 | UCUUUUGGUAGCGUGACUCU | 2121 |
| 1736374 | uscsa | | aasgsa | | UCU | |
| AD- | usgsaacaGfgAfAfAfagaugua | 2393 | VPusUfsuuaCfaUfCfuuuuCfcUfguu | 1853 | AAUGAACAGGAAAAGAUGUA | 2122 |
| 1736375 | asasa | | casusu | | AAA | |
| AD- | gsgsgaacCfaAfGfAfgguauau | 2394 | VPusCfscauAfuAfCfcucuUfgGfuuc | 1854 | GUGGGAACCAAGAGGUAUAU | 2123 |
| 1736376 | gsgsa | | ccsasc | | GGC | |
| AD- | usgsgcacUfaGfGfUfccaaauc | 2395 | VPusAfsagaUfuUfGfgaccUfaGfugc | 1855 | ACUGGCACUAGGUCCAAAUC | 2124 |
| 1736377 | ususa | | casgsu | | UUG | |
| AD- | csascaccUfuCfAfUfauggaga | 2396 | VPusAfsaucUfcCfAfuaugAfaGfgug | 1856 | GGCACACCUUCAUAUGGAGA | 2125 |
| 1736378 | ususa | | ugscsc | | UUG | |
| AD- | csusgguuUfaCfUfGfggaaaua | 2397 | VPusGfscuaUfuUfCfccagUfaAfacc | 1857 | GUCUGGUUUACUGGGAAAUA | 2126 |
| 1736379 | gscsa | | agsasc | | GCC | |
| AD- | gscsccaaAfgGfAfAfuguauau | 2398 | VPusUfsuauAfuAfCfauucCfuUfugg | 1858 | AGGCCCAAAGGAAUGUAUAU | 2127 |
| 1736380 | asasa | | gcscsu | | AAG | |
| AD- | ususaacuGfcAfAfGfaguuuac | 2399 | VPusCfsaguAfaAfCfucuuGfcAfguu | 1859 | UUUUAACUGCAAGAGUUUAC | 2128 |
| 1736381 | usgsa | | aasasa | | UGU | |
| AD- | gsasaccaCfuCfUfCfugagugc | 2400 | VPusUfsugcAfcUfCfagagAfgUfggu | 1860 | AGGAACCACUCUCUGAGUGC | 2129 |
| 1736382 | asasa | | ucscsu | | AAU | |
| AD- | ususaucuUfuUfCfAfuccuggc | 2401 | VPusAfsugcCfaGfGfaugaAfaAfgau | 1861 | CUUUAUCUUUUCAUCCUGGC | 2130 |
| 1736383 | asusa | | aasasg | | AUU | |
| AD- | gsusgaguGfuUfGfGfuaugcca | 2402 | VPusGfsuugGfcAfUfaccaAfcAfcuc | 1862 | GCGUGAGUGUUGGUAUGCCA | 2131 |
| 1736384 | ascsa | | acsgsc | | ACG | |
| AD- | asgsaaugUfaUfUfUfggcuaaa | 2403 | VPusUfsauuUfaGfCfcaaaUfaCfauu | 1863 | UGAGAAUGUAUUUGGCUAAA | 2132 |
| 1736385 | usasa | | cuscsa | | UAA | |
| AD- | gsasacccUfuUfUfAfuuaagag | 2404 | VPusUfsccuCfuUfAfauaaAfaGfggu | 1864 | UGGAACCCUUUUAUUAAGAG | 2133 |
| 1736386 | gsasa | | ucscsa | | GAG | |
| AD- | csusccugUfcCfAfUfagcugcg | 2405 | VPusAfsucgCfaGfCfuaugGfaCfagg | 1865 | GCCUCCUGUCCAUAGCUGCG | 2134 |
| 1736387 | asusa | | agsgsc | | AUG | |
| AD- | ascsugagUfuGfAfAfauaagga | 2406 | VPusCfsuucCfuUfAfuuucAfaCfuca | 1866 | UUACUGAGUUGAAAUAAGGA | 2135 |
| 1736388 | asgsa | | gusasa | | AGG | |
| AD- | csusgccaAfaCfAfGfaaggagc | 2407 | VPusAfsugcUfcCfUfucugUfuUfggc | 1867 | ACCUGCCAAACAGAAGGAGC | 2136 |
| 1736389 | asusa | | agsgsu | | AUG | |
| AD- | gsgscaaaGfuUfGfUfgaagcac | 2408 | VPusGfsaguGfcUfUfcacaAfcUfuug | 1868 | GUGGCAAAGUUGUGAAGCAC | 2137 |
| 1736390 | uscsa | | ccsasc | | UCC | |
| AD- | asusaggaUfgAfUfGfaaguuua | 2409 | VPusUfscuaAfaCfUfucauCfaUfccu | 1869 | AAAUAGGAUGAUGAAGUUUA | 2138 |
| 1736391 | gsasa | | aususu | | GAG | |
| AD- | asusucguAfuCfUfUfaaaaugg | 2410 | VPusUfsgccAfuUfUfuaagAfuAfcga | 1870 | GUAUUCGUAUCUUAAAAUGG | 2139 |
| 1736392 | csasa | | ausasc | | CAC | |
| AD- | csascagcUfuGfGfGfuaauagc | 2411 | VPusAfscgcUfaUfUfacccAfaGfcug | 1871 | AGCACAGCUUGGGUAAUAGC | 2140 |
| 1736393 | gsusa | | ugscsu | | GUU | |
| AD- | ususugcaAfcAfAfCfauaacac | 2412 | VPusCfsaguGfuUfAfuguuGfuUfgca | 1872 | UUUUUGCAACAACAUAACAC | 2141 |
| 1736394 | usgsa | | aasasa | | UGC | |
| AD- | usgsccacCfaUfGfUfgacuuau | 2413 | VPusCfsaauAfaGfUfcacaUfgGfugg | 1873 | UUUGCCACCAUGUGACUUAU | 2142 |
| 1736395 | usgsa | | casasa | | UGG | |
| AD- | uscsgauaGfaGfGfAfaaugaga | 2414 | VPusUfsuucUfcAfUfuuccUfcUfauc | 1874 | CCUCGAUAGAGGAAAUGAGA | 2143 |
| 1736396 | asasa | | gasgsg | | AAG | |
| AD- | ususuuuaAfgUfUfAfgcaggac | 2415 | VPusAfsaguCfcUfGfcuaaCfuUfaaa | 1875 | CCUUUUUAAGUUAGCAGGAC | 2144 |
| 1736397 | ususa | | aasgsg | | UUU | |
| AD- | usgsuaaaUfaAfUfUfaucugcc | 2416 | VPusUfsaggCfaGfAfuaauUfaUfuua | 1876 | GAUGUAAAUAAUUAUCUGCC | 2145 |
| 1736398 | usasa | | casusc | | UAA | |
| AD- | usgsguugUfaCfGfUfgccucaa | 2417 | VPusAfsuuuGfaGfGfcacgUfaCfaac | 1877 | UAUGGUUGUACGUGCCUCAA | 2146 |
| 1736399 | asusa | | casusa | | AUA | |
| AD- | cscsacacUfgAfCfUfagagcca | 2418 | VPusGfsuugGfcUfCfuaguCfaGfugu | 2419 | GCCCACACUGACUAGAGCCA | 2420 |
| 1736400 | ascsa | | ggsgsc | | ACC | |

TABLE 8
Unmodified Sense and Antisense Strand Sequences of PLIN1 dsRNA
Agents Comprising anUnsaturated C22 Hydrocarbon Chain Conjugated
to Position 6 on the Sense Strand, Counting from the 5′-end of
the Sense Strand

| | | SEQ | | | SEQ | |
|---|---|---|---|---|---|---|
| Duplex | Sense Sequence | ID | Range in | Antisense Sequence | ID | Range in |
| Name | 5′ to 3′ | NO: | NM_002666.5 | 5′ to 3′ | NO: | NM_002666.5 |
| AD- | CAUCUCUUUAACCAAACUUGA | 2421 | 1995-2015 | UCAAGUUUGGUUAAAGAGAUGAA | 2556 | 1993-2015 |
| 1735051 | | | | | | |
| AD- | CUCUAACAAAUAAACAGAACA | 2422 | 1740-1760 | UGUUCUGUUUAUUUGUUAGAGAA | 2557 | 1738-1760 |
| 1735052 | | | | | | |
| AD- | UGCAUAGUCACUCUUUUGAUA | 2423 | 2390-2410 | UAUCAAAAGAGUGACUAUGCAGG | 2558 | 2388-2410 |
| 1735053 | | | | | | |
| AD- | AACUUGAUUUUUCAUCUCUUA | 2424 | 1983-2003 | UAAGAGAUGAAAAAUCAAGUUAG | 2559 | 1981-2003 |
| 1735054 | | | | | | |
| AD- | GACACAUUCUUAGCACUGAAA | 2425 | 2128-2148 | UUUCAGUGCUAAGAAUGUGUCAA | 2560 | 2126-2148 |
| 1735055 | | | | | | |
| AD- | ACGCCUUAUUUGAUUUAACUA | 2426 | 2807-2827 | UAGUUAAAUCAAAUAAGGCGUAU | 2561 | 2805-2827 |
| 1735056 | | | | | | |
| AD- | UAGUCUUCGAAAUGUUAAUAA | 2427 | 2474-2494 | UUAUUAACAUUUCGAAGACUAGG | 2562 | 2472-2494 |
| 1735057 | | | | | | |
| AD- | GAUGGACUUUUAAGUUGUUUA | 2428 | 2838-2858 | UAAACAACUUAAAAGUCCAUCAU | 2563 | 2836-2858 |
| 1735058 | | | | | | |
| AD- | UGCUUUUUUCACUUAAUAAUA | 2429 | 2702-2722 | UAUUAUUAAGUGAAAAAAGCAGG | 2564 | 2700-2722 |
| 1735059 | | | | | | |
| AD- | ACAUAAUAACUACUGCAUAAA | 2430 | 2780-2800 | UUUAUGCAGUAGUUAUUAUGUGG | 2565 | 2778-2800 |
| 1735060 | | | | | | |
| AD- | UACUAGUGUCACUUUCUGAGA | 2431 | 2271-2291 | UCUCAGAAAGUGACACUAGUAUU | 2566 | 2269-2291 |
| 1735061 | | | | | | |
| AD- | CAAUCAGAUGCAAAAGCUCUA | 2432 | 2234-2254 | UAGAGCUUUUGCAUCUGAUUGUU | 2567 | 2232-2254 |
| 1735062 | | | | | | |
| AD- | UAAGAGUAAUUGCCUAACUUA | 2433 | 1968-1988 | UAAGUUAGGCAAUUACUCUUAUA | 2568 | 1966-1988 |
| 1735063 | | | | | | |
| AD- | GGCUUGUUUAUGAACAUUAAA | 2434 | 82-102 | UUUAAUGUUCAUAAACAAGCCAU | 2569 | 80-102 |
| 1735064 | | | | | | |
| AD- | UUUUUGCUACUGCAAACGAUA | 2435 | 2871-2891 | UAUCGUUUGCAGUAGCAAAAAAG | 2570 | 2869-2891 |
| 1735065 | | | | | | |
| AD- | AACGAUGCUAUAAUAAAUGUA | 2436 | 2885-2905 | UACAUUUAUUAUAGCAUCGUUUG | 2571 | 2883-2905 |
| 1735066 | | | | | | |
| AD- | UGGUUGUUACUAUAAGAGUAA | 2437 | 1956-1976 | UUACUCUUAUAGUAACAACCAAG | 2572 | 1954-1976 |
| 1735067 | | | | | | |
| AD- | CUGAUGAACAUCCUCUGAUGA | 2438 | 2181-2201 | UCAUCAGAGGAUGUUCAUCAGAG | 2573 | 2179-2201 |
| 1735068 | | | | | | |
| AD- | CUGCGGAUAAAUAUUUGCCAA | 2439 | 2059-2079 | UUGGCAAAUAUUUAUCCGCAGAG | 2574 | 2057-2079 |
| 1735069 | | | | | | |
| AD- | CCAAAAUUCAGAUUCUGCCUA | 2440 | 2039-2059 | UAGGCAGAAUCUGAAUUUUGGAA | 2575 | 2037-2059 |
| 1735070 | | | | | | |
| AD- | AAGGAUGUGUGUCUUUCUCCA | 2441 | 2547-2567 | UGGAGAAAGACACACAUCCUUUU | 2576 | 2545-2567 |
| 1735071 | | | | | | |
| AD- | UGCGAAUGCUUCCAGAAGACA | 2442 | 231-251 | UGUCUUCUGGAAGCAUUCGCAGG | 2577 | 229-251 |
| 1735072 | | | | | | |
| AD- | CGAAUGAGUAACUCCUGUCAA | 2443 | 2079-2099 | UUGACAGGAGUUACUCAUUCGUG | 2578 | 2077-2099 |
| 1735073 | | | | | | |
| AD- | UGGGUGUUUAUUUAAAAUACA | 2444 | 2254-2274 | UGUAUUUUAAAUAAACACCCAAG | 2579 | 2252-2274 |
| 1735074 | | | | | | |
| AD- | CAAAAGAUAUUUGACCGUUUA | 2445 | 2019-2039 | UAAACGGUCAAAUAUCUUUUGGC | 2580 | 2017-2039 |
| 1735075 | | | | | | |
| AD- | AUACAGCUUGGAGAGAUUUUA | 2446 | 2720-2740 | UAAAAUCUCUCCAAGCUGUAUUA | 2581 | 2718-2740 |
| 1735076 | | | | | | |
| AD- | AAAUGCAUUCAUACAAUUACA | 2447 | 2509-2529 | UGUAAUUGUAUGAAUGCAUUUUC | 2582 | 2507-2529 |
| 1735077 | | | | | | |
| AD- | UCCCACUCGCUCUUUUUGAUA | 2448 | 2756-2776 | UAUCAAAAAGAGCGAGUGGGAUU | 2583 | 2754-2776 |
| 1735078 | | | | | | |
| AD- | GCUUGUGUUUACACAAGCUGA | 2449 | 2664-2684 | UCAGCUUGUGUAAACACAAGCGG | 2584 | 2662-2684 |
| 1735079 | | | | | | |
| AD- | CCUGCCUUACAUGGCUUGUUA | 2450 | 70-90 | UAACAAGCCAUGUAAGGCAGGUG | 2585 | 68-90 |
| 1735080 | | | | | | |
| AD- | GAGAUUUUUGUAUCACAUUAA | 2451 | 2732-2752 | UUAAUGUGAUACAAAAAUCUCUC | 2586 | 2730-2752 |
| 1735081 | | | | | | |
| AD- | AGCACUUCAGACAAGGUCCUA | 2452 | 540-560 | UAGGACCUUGUCUGAAGUGCUCG | 2587 | 538-560 |
| 1735082 | | | | | | |
| AD- | UCACUCUCAGAGCCAGUUUUA | 2453 | 1895-1915 | UAAAACUGGCUCUGAGAGUGAAG | 2588 | 1893-1915 |
| 1735083 | | | | | | |
| AD- | ACUGAACUCCUCUGUGAUCUA | 2454 | 2142-2162 | UAGAUCACAGAGGAGUUCAGUGC | 2589 | 2140-2162 |
| 1735084 | | | | | | |
| AD- | UAGUUCCCUAAUGAUGGACUA | 2455 | 2826-2846 | UAGUCCAUCAUUAGGGAACUAGU | 2590 | 2824-2846 |
| 1735085 | | | | | | |
| AD- | GGCGUUACUGACAACGUGGUA | 2456 | 1296-1316 | UACCACGUUGUCAGUAACGCCCU | 2591 | 1294-1316 |
| 1735086 | | | | | | |
| AD- | UGAUCUAGGAUGAUCUGUUCA | 2457 | 2156-2176 | UGAACAGAUCAUCCUAGAUCACA | 2592 | 2154-2176 |
| 1735087 | | | | | | |
| AD- | GUGAUGAUAUAUAGACUUUAA | 2458 | 2427-2447 | UUAAAGUCUAUAUAUCAUCACCA | 2593 | 2425-2447 |
| 1735088 | | | | | | |
| AD- | GGCUACUUUGAAGGGAACAAA | 2459 | 2217-2237 | UUUGUUCCCUUCAAAGUAGCCUG | 2594 | 2215-2237 |
| 1735089 | | | | | | |
| AD- | CUGCUCUGAUUCUAUGGCUUA | 2460 | 1937-1957 | UAAGCCAUAGAAUCAGAGCAGGC | 2595 | 1935-1957 |
| 1735090 | | | | | | |
| AD- | AGAUUGCUUCUGAGCUGAAGA | 2461 | 463-483 | UCUUCAGCUCAGAAGCAAUCUUU | 2596 | 461-483 |
| 1735091 | | | | | | |
| AD- | GCCAUCUCUUGUGUAUGCAGA | 2462 | 2607-2627 | UCUGCAUACACAAGAGAUGGCAC | 2597 | 2605-2627 |
| 1735092 | | | | | | |
| AD- | AGCCACAUUUCCAUUUGCAUA | 2463 | 2354-2374 | UAUGCAAAUGGAAAUGUGGCUGU | 2598 | 2352-2374 |
| 1735093 | | | | | | |
| AD- | UCCAGACAGAAGGUUUUGACA | 2464 | 2111-2131 | UGUCAAAACCUUCUGUCUGGACC | 2599 | 2109-2131 |
| 1735094 | | | | | | |
| AD- | CUCUCGAUACACCGUGCAGAA | 2465 | 818-838 | UUCUGCACGGUGUAUCGAGAGAG | 2600 | 816-838 |
| 1735095 | | | | | | |
| AD- | GGCGCACUUUUUAUUUUUAUA | 2466 | 1838-1858 | UAUAAAAAUAAAAAGUGCGCCUU | 2601 | 1836-1858 |
| 1735096 | | | | | | |
| AD- | GAGCGUUGCCACUUUCAAAGA | 2467 | 1775-1795 | UCUUUGAAAGUGGCAACGCUCGC | 2602 | 1773-1795 |
| 1735097 | | | | | | |
| AD- | UUGCAUCAUUACUGCCUUCAA | 2468 | 2368-2388 | UUGAAGGCAGUAAUGAUGCAAAU | 2603 | 2366-2388 |
| 1735098 | | | | | | |
| AD- | GCGCAAGAAGAGCUGAGUCGA | 2469 | 1685-1705 | UCGACUCAGCUCUUCUUGCGCAG | 2604 | 1683-1705 |
| 1735099 | | | | | | |
| AD- | UGCGGAAUUUGCUGCCAACAA | 2470 | 611-631 | UUGUUGGCAGCAAAUUCCGCAGU | 2605 | 609-631 |
| 1735100 | | | | | | |
| AD- | GUGUGCAAUGCCUAUGAGAAA | 2471 | 291-311 | UUUCUCAUAGGCAUUGCACACAG | 2606 | 289-311 |
| 1735101 | | | | | | |
| AD- | GAAUUCAAAUAUUGCAAAAGA | 2472 | 2530-2550 | UCUUUUGCAAUAUUUGAAUUCUG | 2607 | 2528-2550 |
| 1735102 | | | | | | |
| AD- | CCUGUGUGCUUUGUAGAGCCA | 2473 | 2320-2340 | UGGCUCUACAAAGCACACAGGCC | 2608 | 2318-2340 |
| 1735103 | | | | | | |
| AD- | UCCAGACAAGGAAGAGUCAGA | 2474 | 710-730 | UCUGACUCUUCCUUGUCUGGAGG | 2609 | 708-730 |
| 1735104 | | | | | | |
| AD- | UGCCAGAAACAGCAUCAGCGA | 2475 | 509-529 | UCGCUGAUGCUGUUUCUGGCACU | 2610 | 507-529 |
| 1735105 | | | | | | |
| AD- | GCAUAAUAUGGAUACGCCUUA | 2476 | 2794-2814 | UAAGGCGUAUCCAUAUUAUGCAG | 2611 | 2792-2814 |
| 1735106 | | | | | | |
| AD- | AGCCUCAUCCUGAUGAUGCUA | 2477 | 1813-1833 | UAGCAUCAUCAGGAUGAGGCUGA | 2612 | 1811-1833 |
| 1735107 | | | | | | |
| AD- | AAGCCUCUUGAGCAGGGUUGA | 2478 | 779-799 | UCAACCCUGCUCAAGAGGCUUGG | 2613 | 777-799 |
| 1735108 | | | | | | |
| AD- | UAAAGGGAAGAAGUUGAAGCA | 2479 | 99-119 | UGCUUCAACUUCUUCCCUUUAAU | 2614 | 97-119 |
| 1735109 | | | | | | |
| AD- | GGCAGCAUUGAGAAGGUGGUA | 2480 | 675-695 | UACCACCUUCUCAAUGCUGCCCA | 2615 | 673-695 |
| 1735110 | | | | | | |
| AD- | GUCAGCGACAGCUUCUUCCGA | 2481 | 1617-1637 | UCGGAAGAAGCUGUCGCUGACCC | 2616 | 1615-1637 |
| 1735111 | | | | | | |
| AD- | CUGCUGUCUCCUCAACCAAGA | 2482 | 1243-1263 | UCUUGGUUGAGGAGACAGCAGGG | 2617 | 1241-1263 |
| 1735112 | | | | | | |
| AD- | AGGAGGAAGAAUUGGAGACUA | 2483 | 1054-1074 | UAGUCUCCAAUUCUUCCUCCUCC | 2618 | 1052-1074 |
| 1735113 | | | | | | |
| AD- | CCUGUCACCACUCUGAAGGUA | 2484 | 2092-2112 | UACCUUCAGAGUGGUGACAGGAG | 2619 | 2090-2112 |
| 1735114 | | | | | | |
| AD- | AGACUUUAUGUAUAGCCACAA | 2485 | 2439-2459 | UUGUGGCUAUACAUAAAGUCUAU | 2620 | 2437-2459 |
| 1735115 | | | | | | |
| AD- | GUUACGCAUGCCUGCUUUUUA | 2486 | 2690-2710 | UAAAAAGCAGGCAUGCGUAACAC | 2621 | 2688-2710 |
| 1735116 | | | | | | |
| AD- | CCAGUUCACAGCUGCCAAUGA | 2487 | 377-397 | UCAUUGGCAGCUGUGAACUGGGU | 2622 | 375-397 |
| 1735117 | | | | | | |
| AD- | CAGGCACGUGUGUAUGCACUA | 2488 | 2640-2660 | UAGUGCAUACACACGUGCCUGCA | 2623 | 2638-2660 |
| 1735118 | | | | | | |
| AD- | AUGGCAGUCAACAAAGGCCUA | 2489 | 132-152 | UAGGCCUUUGUUGACUGCCAUCC | 2624 | 130-152 |
| 1735119 | | | | | | |
| AD- | CAAACUUGUGGCCAAAAGAUA | 2490 | 2007-2027 | UAUCUUUUGGCCACAAGUUUGGU | 2625 | 2005-2027 |
| 1735120 | | | | | | |
| AD- | UCUGACCAACACCCUCUCUCA | 2491 | 803-823 | UGAGAGAGGGUGUUGGUCAGAGC | 2626 | 801-823 |
| 1735121 | | | | | | |
| AD- | UCACAUUAUAAAUCCCACUCA | 2492 | 2744-2764 | UGAGUGGGAUUUAUAAUGUGAUA | 2627 | 2742-2764 |
| 1735122 | | | | | | |
| AD- | GAGGAGAACAAGUUCAGUGAA | 2493 | 1074-1094 | UUCACUGAACUUGUUCUCCUCAG | 2628 | 1072-1094 |
| 1735123 | | | | | | |
| AD- | GAGCAGGAGAAUGUGCUGCAA | 2494 | 177-197 | UUGCAGCACAUUCUCCUGCUCAG | 2629 | 175-197 |
| 1735124 | | | | | | |
| AD- | GAAGACCUACACCAGCACUAA | 2495 | 245-265 | UUAGUGCUGGUGUAGGUCUUCUG | 2630 | 243-265 |
| 1735125 | | | | | | |
| AD- | AGGCCUCACCUUGCUGGAUGA | 2496 | 146-166 | UCAUCCAGCAAGGUGAGGCCUUU | 2631 | 144-166 |
| 1735126 | | | | | | |
| AD- | AGCCAGCUGCUUGCUCACAGA | 2497 | 2336-2356 | UCUGUGAGCAAGCAGCUGGCUCU | 2632 | 2334-2356 |
| 1735127 | | | | | | |
| AD- | GGGCUCUGUGAGACUGAGGUA | 2498 | 13-33 | UACCUCAGUCUCACAGAGCCCAG | 2633 | Nov-33 |
| 1735128 | | | | | | |
| AD- | CGAGAGCGAAUUCCGGGACAA | 2499 | 1367-1387 | UUGUCCCGGAAUUCGCUCUCGGG | 2634 | 1365-1387 |
| 1735129 | | | | | | |
| AD- | GUGGCACAUACCCUGCAGAAA | 2500 | 1134-1154 | UUUCUGCAGGGUAUGUGCCACAC | 2635 | 1132-1154 |
| 1735130 | | | | | | |
| AD- | GUGGACACAGUGGUGCAUUAA | 2501 | 1314-1334 | UUAAUGCACCACUGUGUCCACCA | 2636 | 1312-1334 |
| 1735131 | | | | | | |
| AD- | AAGCCCAGAAGUCUCCCAAGA | 2502 | 751-771 | UCUUGGGAGACUUCUGGGCUUGC | 2637 | 749-771 |
| 1735132 | | | | | | |
| AD- | GGCCAUGUCCCUAUCAGAUGA | 2503 | 1268-1288 | UCAUCUGAUAGGGACAUGGCCCU | 2638 | 1266-1288 |
| 1735133 | | | | | | |
| AD- | GCCGGCGGGUUUCUCUAACAA | 2504 | 1728-1748 | UUGUUAGAGAAACCCGCCGGCCC | 2639 | 1726-1748 |
| 1735134 | | | | | | |
| AD- | CUGAAGGACACCAUCUCCACA | 2505 | 477-497 | UGUGGAGAUGGUGUCCUUCAGCU | 2640 | 475-497 |
| 1735135 | | | | | | |
| AD- | GACCACCAUCUCGGCUGUGAA | 2506 | 1163-1183 | UUCACAGCCGAGAUGGUGGUCUG | 2641 | 1161-1183 |
| 1735136 | | | | | | |
| AD- | CCAGUUUUUAAGGGACACCAA | 2507 | 1907-1927 | UUGGUGUCCCUUAAAAACUGGCU | 2642 | 1905-1927 |
| 1735137 | | | | | | |
| AD- | GGCCAGAGACACUGCGGAAUA | 2508 | 599-619 | UAUUCCGCAGUGUCUCUGGCCAC | 2643 | 597-619 |
| 1735138 | | | | | | |
| AD- | CGUUCCCAUCGCGAGCACUUA | 2509 | 527-547 | UAAGUGCUCGCGAUGGGAACGCU | 2644 | 525-547 |
| 1735139 | | | | | | |
| AD- | CCGGGACAUCGACAACCCACA | 2510 | 1379-1399 | UGUGGGUUGUCGAUGUCCCGGAA | 2645 | 1377-1399 |
| 1735140 | | | | | | |
| AD- | UCUGAUGAUCUAGGCUCCCAA | 2511 | 2194-2214 | UUGGGAGCCUAGAUCAUCAGAGG | 2646 | 2192-2214 |
| 1735141 | | | | | | |
| AD- | CUGCAGAAGACCCUCCAGACA | 2512 | 1146-1166 | UGUCUGGAGGGUCUUCUGCAGGG | 2647 | 1144-1166 |
| 1735142 | | | | | | |
| AD- | CGCCAGUAGCUUGGCUGCCUA | 2513 | 323-343 | UAGGCAGCCAAGCUACUGGCGCU | 2648 | 321-343 |
| 1735143 | | | | | | |
| AD- | CGCUUCACAGGCUGAGUCCAA | 2514 | 2298-2318 | UUGGACUCAGCCUGUGAAGCGGC | 2649 | 2296-2318 |
| 1735144 | | | | | | |
| AD- | UUUUUUUAGCAUCCUUUUGGA | 2515 | 1871-1891 | UCCAAAAGGAUGCUAAAAAAAAA | 2650 | 1869-1891 |
| 1735145 | | | | | | |
| AD- | GAAGCCAAAGCGCAGGGUCAA | 2516 | 1601-1621 | UUGACCCUGCGCUUUGGCUUCUC | 2651 | 1599-1621 |
| 1735146 | | | | | | |
| AD- | GAGGAGGAUCAUGAGGACCAA | 2517 | 1005-1025 | UUGGUCCUCAUGAUCCUCCUCCU | 2652 | 1003-1025 |
| 1735147 | | | | | | |
| AD- | GCACGCAUUACAGCCAGCUGA | 2518 | 1666-1686 | UCAGCUGGCUGUAAUGCGUGCGG | 2653 | 1664-1686 |
| 1735148 | | | | | | |
| AD- | CUUCAUUGAAAACCACCACGA | 2519 | 2585-2605 | UCGUGGUGGUUUUCAAUGAAGGG | 2654 | 2583-2605 |
| 1735149 | | | | | | |
| AD- | UUUUUGAUGGCCACAUAAUAA | 2520 | 2768-2788 | UUAUUAUGUGGCCAUCAAAAAGA | 2655 | 2766-2788 |
| 1735150 | | | | | | |
| AD- | CCACCUGGAGGAAAAGAUCCA | 2521 | 419-439 | UGGAUCUUUUCCUCCAGGUGGUC | 2656 | 417-439 |
| 1735151 | | | | | | |
| AD- | CAAGGCCAAGCCAAGCCUCUA | 2522 | 767-787 | UAGAGGCUUGGCUUGGCCUUGGG | 2657 | 765-787 |
| 1735152 | | | | | | |
| AD- | GAGGACCAGACAGACACGGAA | 2523 | 1017-1037 | UUCCGUGUCUGUCUGGUCCUCAU | 2658 | 1015-1037 |
| 1735153 | | | | | | |
| AD- | GAUGCCCUGAAGGGCGUUACA | 2524 | 1284-1304 | UGUAACGCCCUUCAGGGCAUCUG | 2659 | 1282-1304 |
| 1735154 | | | | | | |
| AD- | CCAACACUCGAGCUGGCCGAA | 2525 | 625-645 | UUCGGCCAGCUCGAGUGUUGGCA | 2660 | 623-645 |
| 1735155 | | | | | | |
| AD- | CAGGGCUAUGCACCUGCAGGA | 2526 | 2624-2644 | UCCUGCAGGUGCAUAGCCCUGCA | 2661 | 2622-2644 |
| 1735156 | | | | | | |
| AD- | AAGGUGGUGGAGUACCUCCUA | 2527 | 687-707 | UAGGAGGUACUCCACCACCUUCU | 2662 | 685-707 |
| 1735157 | | | | | | |
| AD- | UUGAAGCUUGAGGAGCGAGGA | 2528 | 112-132 | UCCUCGCUCCUCAAGCUUCAACU | 2663 | 110-132 |
| 1735158 | | | | | | |
| AD- | CGUGGCCAUGUGGAUCCCAGA | 2529 | 869-889 | UCUGGGAUCCACAUGGCCACGGU | 2664 | 867-889 |
| 1735159 | | | | | | |
| AD- | CAGCACUAAGGAAGCCCACCA | 2530 | 257-277 | UGGUGGGCUUCCUUAGUGCUGGU | 2665 | 255-277 |
| 1735160 | | | | | | |
| AD- | GUCGCUGAUGGAGCCCGAGAA | 2531 | 1352-1372 | UUCUCGGGCUCCAUCAGCGACAG | 2666 | 1350-1372 |
| 1735161 | | | | | | |
| AD- | UUCUUCCGGCCCAGCGUCAUA | 2532 | 1629-1649 | UAUGACGCUGGGCCGGAAGAAGC | 2667 | 1627-1649 |
| 1735162 | | | | | | |
| AD- | CUGCUCCUGGACACCAGCAAA | 2533 | 733-753 | UUUGCUGGUGUCCAGGAGCAGGG | 2668 | 731-753 |
| 1735163 | | | | | | |
| AD- | UUUCUGAGUACCCGCCGCUUA | 2534 | 2283-2303 | UAAGCGGCGGGUACUCAGAAAGU | 2669 | 2281-2303 |
| 1735164 | | | | | | |
| AD- | CGCAGGCUGUCCACCCAGUUA | 2535 | 363-383 | UAACUGGGUGGACAGCCUGCGGA | 2670 | 361-383 |
| 1735165 | | | | | | |
| AD- | GGCUGUGACAUGGGCACCUGA | 2536 | 1175-1195 | UCAGGUGCCCAUGUCACAGCCGA | 2671 | 1173-1195 |
| 1735166 | | | | | | |
| AD- | UCAGUGAGGUAGCAGCCCUGA | 2537 | 1087-1107 | UCAGGGCUGCUACCUCACUGAAC | 2672 | 1085-1107 |
| 1735167 | | | | | | |
| AD- | AGCGUCAUGGAGCCCAUCCUA | 2538 | 1641-1661 | UAGGAUGGGCUCCAUGACGCUGG | 2673 | 1639-1661 |
| 1735168 | | | | | | |
| AD- | CUGGAUGGAGACCUCCCUGAA | 2539 | 159-179 | UUCAGGGAGGUCUCCAUCCAGCA | 2674 | 157-179 |
| 1735169 | | | | | | |
| AD- | UCAGCCGGAGUGAGUGUUGGA | 2540 | 38-58 | UCCAACACUCACUCCGGCUGACC | 2675 | 36-58 |
| 1735170 | | | | | | |
| AD- | CUGGCCGACUGGCUUCUGGAA | 2541 | 637-657 | UUCCAGAAGCCAGUCGGCCAGCU | 2676 | 635-657 |
| 1735171 | | | | | | |
| AD- | GGUGCUGCACCUCACACCAGA | 2542 | 1220-1240 | UCUGGUGUGAGGUGCAGCACCCU | 2677 | 1218-1240 |
| 1735172 | | | | | | |
| AD- | GGCUGCCUGGAGCAUGGAGCA | 2543 | 335-355 | UGCUCCAUGCUCCAGGCAGCCAA | 2678 | 333-355 |
| 1735173 | | | | | | |
| AD- | CCUGCAGCUGUGCUGGGCAUA | 2544 | 1191-1211 | UAUGCCCAGCACAGCUGCAGGUG | 2679 | 1189-1211 |
| 1735174 | | | | | | |
| AD- | AGUGGCCAUGCAGGCGGUGUA | 2545 | 929-949 | UACACCGCCUGCAUGGCCACUGA | 2680 | 927-949 |
| 1735175 | | | | | | |
| AD- | GACUUGGCCUUGGGCAGCAUA | 2546 | 663-683 | UAUGCUGCCCAAGGCCAAGUCGG | 2681 | 661-683 |
| 1735176 | | | | | | |
| AD- | GAGCGAAGUGCGGGUACCCUA | 2547 | 959-979 | UAGGGUACCCGCACUUCGCUCCU | 2682 | 957-979 |
| 1735177 | | | | | | |
| AD- | CUGGUGGCCUCUGUGUGCAAA | 2548 | 279-299 | UUUGCACACAGAGGCCACCAGGG | 2683 | 277-299 |
| 1735178 | | | | | | |
| AD- | AACCCACCAGCCGAGGUCGAA | 2549 | 1392-1412 | UUCGACCUCGGCUGGUGGGUUGU | 2684 | 1390-1412 |
| 1735179 | | | | | | |
| AD- | UGAUGCUGCCAAGGCGCACUA | 2550 | 1826-1846 | UAGUGCGCCUUGGCAGCAUCAUC | 2685 | 1824-1846 |
| 1735180 | | | | | | |
| AD- | CCAAUGAGCUGGCCUGCCGAA | 2551 | 391-411 | UUCGGCAGGCCAGCUCAUUGGCA | 2686 | 389-411 |
| 1735181 | | | | | | |
| AD- | GUGCUGCAGCGGGUCCUGCAA | 2552 | 189-209 | UUGCAGGACCCGCUGCAGCACAU | 2687 | 187-209 |
| 1735182 | | | | | | |
| AD- | GGGCGUGCAGAGCGCCAGUAA | 2553 | 311-331 | UUACUGGCGCUCUGCACGCCCUU | 2688 | 309-331 |
| 1735183 | | | | | | |
| AD- | GGUGAGUGGCACCUGCGAAUA | 2554 | 218-238 | UAUUCGCAGGUGCCACUCACCAC | 2689 | 216-238 |
| 1735184 | | | | | | |
| AD- | ACGGAGGGAGAGGACACGGAA | 2555 | 1032-1052 | UUCCGUGUCCUCUCCCUCCGUGU | 2690 | 1030-1052 |
| 1735185 | | | | | | |

TABLE 9
Modified Sense and Antisense Strand Sequences of PLIN1 dsRNA Agents Comprising an Unsaturated C22 Hydrocarbon Chain
Conjugated to Position 6 on the Sense Strand, Counting from the 5′-end of the Sense Strand

| | | SEQ | | SEQ | | SEQ |
|---|---|---|---|---|---|---|
| Duplex | | ID | | ID | | ID |
| Name | Sense Sequence 5′ to 3′ | NO: | Antisense Sequence 5′ to 3′ | NO: | mRNA Target Sequence 5′ to 3′ | NO: |
| AD- | csasucu(Cda)UfuUfAfAfccaaacuusg | 2691 | VPusCfsaagUfuUfGfguuaAfaGfagaugsas | 2826 | UUCAUCUCUUUAACCAAACU | 2961 |
| 1735051 | sa | | a | | UGU | |
| AD- | csuscua(Ada)CfaAfAfUfaaacagaasc | 2692 | VPusGfsuucUfgUfUfuauuUfgUfuagagsas | 2827 | UUCUCUAACAAAUAAACAGA | 2962 |
| 1735052 | sa | | a | | ACC | |
| AD- | usgscau(Ada)GfuCfAfCfucuuuugas | 2693 | VPusAfsucaAfaAfGfagugAfcUfaugcasgs | 2828 | CCUGCAUAGUCACUCUUUUG | 2963 |
| 1735053 | usa | | g | | AUG | |
| AD- | asascuu(Gda)AfuUfUfUfucaucucus | 2694 | VPusAfsagaGfaUfGfaaaaAfuCfaaguusasg | 2829 | CUAACUUGAUUUUUCAUCUC | 2964 |
| 1735054 | usa | | | | UUU | |
| AD- | gsascac(Ada)UfuCfUfUfagcacugasa | 2695 | VPusUfsucaGfuGfCfuaagAfaUfgugucsas | 2830 | UUGACACAUUCUUAGCACUG | 2965 |
| 1735055 | sa | | a | | AAC | |
| AD- | ascsgcc(Uda)UfaUfUfUfgauuuaacsu | 2696 | VPusAfsguuAfaAfUfcaaaUfaAfggcgusas | 2831 | AUACGCCUUAUUUGAUUUAA | 2966 |
| 1735056 | sa | | u | | CUA | |
| AD- | usasguc(Uda)UfcGfAfAfauguuaaus | 2697 | VPusUfsauuAfaCfAfuuucGfaAfgacuasgs | 2832 | CCUAGUCUUCGAAAUGUUAA | 2967 |
| 1735057 | asa | | g | | UAU | |
| AD- | gsasugg(Ada)CfuUfUfUfaaguuguus | 2698 | VPusAfsaacAfaCfUfuaaaAfgUfccaucsasu | 2833 | AUGAUGGACUUUUAAGUUGU | 2968 |
| 1735058 | usa | | | | UUC | |
| AD- | usgscuu(Uda)UfuUfCfAfcuuaauaas | 2699 | VPusAfsuuaUfuAfAfgugaAfaAfaagcasgs | 2834 | CCUGCUUUUUUCACUUAAUA | 2969 |
| 1735059 | usa | | g | | AUA | |
| AD- | ascsaua(Ada)UfaAfCfUfacugcauasa | 2700 | VPusUfsuauGfcAfGfuaguUfaUfuaugusgs | 2835 | CCACAUAAUAACUACUGCAU | 2970 |
| 1735060 | sa | | g | | AAU | |
| AD- | usascua(Gda)UfgUfCfAfcuuucugas | 2701 | VPusCfsucaGfaAfAfgugaCfaCfuaguasusu | 2836 | AAUACUAGUGUCACUUUCUG | 2971 |
| 1735061 | gsa | | | | AGU | |
| AD- | csasauc(Ada)GfaUfGfCfaaaagcucsu | 2702 | VPusAfsgagCfuUfUfugcaUfcUfgauugsus | 2837 | AACAAUCAGAUGCAAAAGCU | 2972 |
| 1735062 | sa | | u | | CUU | |
| AD- | usasaga(Gda)UfaAfUfUfgccuaacusu | 2703 | VPusAfsaguUfaGfGfcaauUfaCfucuuasusa | 2838 | UAUAAGAGUAAUUGCCUAAC | 2973 |
| 1735063 | sa | | | | UUG | |
| AD- | gsgscuu(Gda)UfuUfAfUfgaacauuas | 2704 | VPusUfsuaaUfgUfUfcauaAfaCfaagccsasu | 2839 | AUGGCUUGUUUAUGAACAUU | 2974 |
| 1735064 | asa | | | | AAA | |
| AD- | ususuuu(Gda)CfuAfCfUfgcaaacgas | 2705 | VPusAfsucgUfuUfGfcaguAfgCfaaaaasasg | 2840 | CUUUUUUGCUACUGCAAACG | 2975 |
| 1735065 | usa | | | | AUG | |
| AD- | asascga(Uda)GfcUfAfUfaauaaaugsu | 2706 | VPusAfscauUfuAfUfuauaGfcAfucguusus | 2841 | CAAACGAUGCUAUAAUAAAU | 2976 |
| 1735066 | sa | | g | | GUC | |
| AD- | usgsguu(Gda)UfuAfCfUfauaagagus | 2707 | VPusUfsacuCfuUfAfuaguAfaCfaaccasasg | 2842 | CUUGGUUGUUACUAUAAGAG | 2977 |
| 1735067 | asa | | | | UAA | |
| AD- | csusgau(Gda)AfaCfAfUfccucugaus | 2708 | VPusCfsaucAfgAfGfgaugUfuCfaucagsas | 2843 | CUCUGAUGAACAUCCUCUGA | 2978 |
| 1735068 | gsa | | g | | UGA | |
| AD- | csusgcg(Gda)AfuAfAfAfuauuugccs | 2709 | VPusUfsggcAfaAfUfauuuAfuCfcgcagsas | 2844 | CUCUGCGGAUAAAUAUUUGC | 2979 |
| 1735069 | asa | | g | | CAC | |
| AD- | cscsaaa(Ada)UfuCfAfGfauucugccsu | 2710 | VPusAfsggcAfgAfAfucugAfaUfuuuggsas | 2845 | UUCCAAAAUUCAGAUUCUGC | 2980 |
| 1735070 | sa | | a | | CUC | |
| AD- | asasgga(Uda)GfuGfUfGfucuuucucs | 2711 | VPusGfsgagAfaAfGfacacAfcAfuccuusus | 2846 | AAAAGGAUGUGUGUCUUUCU | 2981 |
| 1735071 | csa | | u | | CCC | |
| AD- | usgscga(Ada)UfgCfUfUfccagaagasc | 2712 | VPusGfsucuUfcUfGfgaagCfaUfucgcasgs | 2847 | CCUGCGAAUGCUUCCAGAAG | 2982 |
| 1735072 | sa | | g | | ACC | |
| AD- | csgsaau(Gda)AfgUfAfAfcuccugucs | 2713 | VPusUfsgacAfgGfAfguuaCfuCfauucgsus | 2848 | CACGAAUGAGUAACUCCUGU | 2983 |
| 1735073 | asa | | g | | CAC | |
| AD- | usgsggu(Gda)UfuUfAfUfuuaaaauas | 2714 | VPusGfsuauUfuUfAfaauaAfaCfacccasasg | 2849 | CUUGGGUGUUUAUUUAAAAU | 2984 |
| 1735074 | csa | | | | ACU | |
| AD- | csasaaa(Gda)AfuAfUfUfugaccguus | 2715 | VPusAfsaacGfgUfCfaaauAfuCfuuuugsgs | 2850 | GCCAAAAGAUAUUUGACCGU | 2985 |
| 1735075 | usa | | c | | UUC | |
| AD- | asusaca(Gda)CfuUfGfGfagagauuus | 2716 | VPusAfsaaaUfcUfCfuccaAfgCfuguaususa | 2851 | UAAUACAGCUUGGAGAGAUU | 2986 |
| 1735076 | usa | | | | UUU | |
| AD- | asasaug(Cda)AfuUfCfAfuacaauuasc | 2717 | VPusGfsuaaUfuGfUfaugaAfuGfcauuusus | 2852 | GAAAAUGCAUUCAUACAAUU | 2987 |
| 1735077 | sa | | c | | ACA | |
| AD- | uscscca(Cda)UfcGfCfUfcuuuuugasu | 2718 | VPusAfsucaAfaAfAfgagcGfaGfugggasus | 2853 | AAUCCCACUCGCUCUUUUUGA | 2988 |
| 1735078 | sa | | u | | UG | |
| AD- | gscsuug(Uda)GfuUfUfAfcacaagcus | 2719 | VPusCfsagcUfuGfUfguaaAfcAfcaagcsgsg | 2854 | CCGCUUGUGUUUACACAAGC | 2989 |
| 1735079 | gsa | | | | UGU | |
| AD- | cscsugc(Cda)UfuAfCfAfuggcuugus | 2720 | VPusAfsacaAfgCfCfauguAfaGfgcaggsus | 2855 | CACCUGCCUUACAUGGCUUGU | 2990 |
| 1735080 | usa | | g | | UU | |
| AD- | gsasgau(Uda)UfuUfGfUfaucacauus | 2721 | VPusUfsaauGfuGfAfuacaAfaAfaucucsusc | 2856 | GAGAGAUUUUUGUAUCACAU | 2991 |
| 1735081 | asa | | | | UAU | |
| AD- | asgscac(Uda)UfcAfGfAfcaagguccsu | 2722 | VPusAfsggaCfcUfUfgucuGfaAfgugcuscs | 2857 | CGAGCACUUCAGACAAGGUCC | 2992 |
| 1735082 | sa | | g | | UG | |
| AD- | uscsacu(Cda)UfcAfGfAfgccaguuus | 2723 | VPusAfsaaaCfuGfGfcucuGfaGfagugasasg | 2858 | CUUCACUCUCAGAGCCAGUUU | 2993 |
| 1735083 | usa | | | | UU | |
| AD- | ascsuga(Ada)CfuCfCfUfcugugaucsu | 2724 | VPusAfsgauCfaCfAfgaggAfgUfucagusgs | 2859 | GCACUGAACUCCUCUGUGAUC | 2994 |
| 1735084 | sa | | c | | UA | |
| AD- | usasguu(Cda)CfcUfAfAfugauggacs | 2725 | VPusAfsgucCfaUfCfauuaGfgGfaacuasgsu | 2860 | ACUAGUUCCCUAAUGAUGGA | 2995 |
| 1735085 | usa | | | | CUU | |
| AD- | gsgscgu(Uda)AfcUfGfAfcaacguggs | 2726 | VPusAfsccaCfgUfUfgucaGfuAfacgccscsu | 2861 | AGGGCGUUACUGACAACGUG | 2996 |
| 1735086 | usa | | | | GUG | |
| AD- | usgsauc(Uda)AfgGfAfUfgaucuguus | 2727 | VPusGfsaacAfgAfUfcaucCfuAfgaucascsa | 2862 | UGUGAUCUAGGAUGAUCUGU | 2997 |
| 1735087 | csa | | | | UCC | |
| AD- | gsusgau(Gda)AfuAfUfAfuagacuuus | 2728 | VPusUfsaaaGfuCfUfauauAfuCfaucacscsa | 2863 | UGGUGAUGAUAUAUAGACUU | 2998 |
| 1735088 | asa | | | | UAU | |
| AD- | gsgscua(Cda)UfuUfGfAfagggaacasa | 2729 | VPusUfsuguUfcCfCfuucaAfaGfuagccsus | 2864 | CAGGCUACUUUGAAGGGAAC | 2999 |
| 1735089 | sa | | g | | AAU | |
| AD- | csusgcu(Cda)UfgAfUfUfcuauggcus | 2730 | VPusAfsagcCfaUfAfgaauCfaGfagcagsgsc | 2865 | GCCUGCUCUGAUUCUAUGGC | 3000 |
| 1735090 | usa | | | | UUG | |
| AD- | asgsauu(Gda)CfuUfCfUfgagcugaas | 2731 | VPusCfsuucAfgCfUfcagaAfgCfaaucususu | 2866 | AAAGAUUGCUUCUGAGCUGA | 3001 |
| 1735091 | gsa | | | | AGG | |
| AD- | gscscau(Cda)UfcUfUfGfuguaugcas | 2732 | VPusCfsugcAfuAfCfacaaGfaGfauggcsasc | 2867 | GUGCCAUCUCUUGUGUAUGC | 3002 |
| 1735092 | gsa | | | | AGG | |
| AD- | asgscca(Cda)AfuUfUfCfcauuugcasu | 2733 | VPusAfsugcAfaAfUfggaaAfuGfuggcusgs | 2868 | ACAGCCACAUUUCCAUUUGCA | 3003 |
| 1735093 | sa | | u | | UC | |
| AD- | uscscag(Ada)CfaGfAfAfgguuuugas | 2734 | VPusGfsucaAfaAfCfcuucUfgUfcuggascsc | 2869 | GGUCCAGACAGAAGGUUUUG | 3004 |
| 1735094 | csa | | | | ACA | |
| AD- | csuscuc(Gda)AfuAfCfAfccgugcagsa | 2735 | VPusUfscugCfaCfGfguguAfuCfgagagsas | 2870 | CUCUCUCGAUACACCGUGCAG | 3005 |
| 1735095 | sa | | g | | AC | |
| AD- | gsgscgc(Ada)CfuUfUfUfuauuuuuas | 2736 | VPusAfsuaaAfaAfUfaaaaAfgUfgcgccsusu | 2871 | AAGGCGCACUUUUUAUUUUU | 3006 |
| 1735096 | usa | | | | AUU | |
| AD- | gsasgcg(Uda)UfgCfCfAfcuuucaaasg | 2737 | VPusCfsuuuGfaAfAfguggCfaAfcgcucsgs | 2872 | GCGAGCGUUGCCACUUUCAA | 3007 |
| 1735097 | sa | | c | | AGU | |
| AD- | ususgca(Uda)CfaUfUfAfcugccuucsa | 2738 | VPusUfsgaaGfgCfAfguaaUfgAfugcaasas | 2873 | AUUUGCAUCAUUACUGCCUU | 3008 |
| 1735098 | sa | | u | | CAC | |
| AD- | gscsgca(Ada)GfaAfGfAfgcugagucs | 2739 | VPusCfsgacUfcAfGfcucuUfcUfugcgcsasg | 2874 | CUGCGCAAGAAGAGCUGAGU | 3009 |
| 1735099 | gsa | | | | CGC | |
| AD- | usgscgg(Ada)AfuUfUfGfcugccaacs | 2740 | VPusUfsguuGfgCfAfgcaaAfuUfccgcasgs | 2875 | ACUGCGGAAUUUGCUGCCAA | 3010 |
| 1735100 | asa | | u | | CAC | |
| AD- | gsusgug(Cda)AfaUfGfCfcuaugagas | 2741 | VPusUfsucuCfaUfAfggcaUfuGfcacacsasg | 2876 | CUGUGUGCAAUGCCUAUGAG | 3011 |
| 1735101 | asa | | | | AAG | |
| AD- | gsasauu(Cda)AfaAfUfAfuugcaaaasg | 2742 | VPusCfsuuuUfgCfAfauauUfuGfaauucsus | 2877 | CAGAAUUCAAAUAUUGCAAA | 3012 |
| 1735102 | sa | | g | | AGG | |
| AD- | cscsugu(Gda)UfgCfUfUfuguagagcs | 2743 | VPusGfsgcuCfuAfCfaaagCfaCfacaggscsc | 2878 | GGCCUGUGUGCUUUGUAGAG | 3013 |
| 1735103 | csa | | | | CCA | |
| AD- | uscscag(Ada)CfaAfGfGfaagagucasg | 2744 | VPusCfsugaCfuCfUfuccuUfgUfcuggasgs | 2879 | CCUCCAGACAAGGAAGAGUC | 3014 |
| 1735104 | sa | | g | | AGC | |
| AD- | usgscca(Gda)AfaAfCfAfgcaucagcsg | 2745 | VPusCfsgcuGfaUfGfcuguUfuCfuggcascs | 2880 | AGUGCCAGAAACAGCAUCAG | 3015 |
| 1735105 | sa | | u | | CGU | |
| AD- | gscsaua(Ada)UfaUfGfGfauacgccusu | 2746 | VPusAfsaggCfgUfAfuccaUfaUfuaugcsas | 2881 | CUGCAUAAUAUGGAUACGCC | 3016 |
| 1735106 | sa | | g | | UUA | |
| AD- | asgsccu(Cda)AfuCfCfUfgaugaugcsu | 2747 | VPusAfsgcaUfcAfUfcaggAfuGfaggcusgs | 2882 | UCAGCCUCAUCCUGAUGAUGC | 3017 |
| 1735107 | sa | | a | | UG | |
| AD- | asasgcc(Uda)CfuUfGfAfgcaggguus | 2748 | VPusCfsaacCfcUfGfcucaAfgAfggcuusgsg | 2883 | CCAAGCCUCUUGAGCAGGGU | 3018 |
| 1735108 | gsa | | | | UGG | |
| AD- | usasaag(Gda)GfaAfGfAfaguugaags | 2749 | VPusGfscuuCfaAfCfuucuUfcCfcuuuasasu | 2884 | AUUAAAGGGAAGAAGUUGAA | 3019 |
| 1735109 | csa | | | | GCU | |
| AD- | gsgscag(Cda)AfuUfGfAfgaagguggs | 2750 | VPusAfsccaCfcUfUfcucaAfuGfcugccscsa | 2885 | UGGGCAGCAUUGAGAAGGUG | 3020 |
| 1735110 | usa | | | | GUG | |
| AD- | gsuscag(Cda)GfaCfAfGfcuucuuccsg | 2751 | VPusCfsggaAfgAfAfgcugUfcGfcugacscs | 2886 | GGGUCAGCGACAGCUUCUUCC | 3021 |
| 1735111 | sa | | c | | GG | |
| AD- | csusgcu(Gda)UfcUfCfCfucaaccaasg | 2752 | VPusCfsuugGfuUfGfaggaGfaCfagcagsgs | 2887 | CCCUGCUGUCUCCUCAACCAA | 3022 |
| 1735112 | sa | | g | | GG | |
| AD- | asgsgag(Gda)AfaGfAfAfuuggagacs | 2753 | VPusAfsgucUfcCfAfauucUfuCfcuccuscsc | 2888 | GGAGGAGGAAGAAUUGGAGA | 3023 |
| 1735113 | usa | | | | CUG | |
| AD- | cscsugu(Cda)AfcCfAfCfucugaaggsu | 2754 | VPusAfsccuUfcAfGfagugGfuGfacaggsas | 2889 | CUCCUGUCACCACUCUGAAGG | 3024 |
| 1735114 | sa | | g | | UC | |
| AD- | asgsacu(Uda)UfaUfGfUfauagccacsa | 2755 | VPusUfsgugGfcUfAfuacaUfaAfagucusas | 2890 | AUAGACUUUAUGUAUAGCCA | 3025 |
| 1735115 | sa | | u | | CAG | |
| AD- | gsusuac(Gda)CfaUfGfCfcugcuuuus | 2756 | VPusAfsaaaAfgCfAfggcaUfgCfguaacsasc | 2891 | GUGUUACGCAUGCCUGCUUU | 3026 |
| 1735116 | usa | | | | UUU | |
| AD- | cscsagu(Uda)CfaCfAfGfcugccaausg | 2757 | VPusCfsauuGfgCfAfgcugUfgAfacuggsgs | 2892 | ACCCAGUUCACAGCUGCCAAU | 3027 |
| 1735117 | sa | | u | | GA | |
| AD- | csasggc(Ada)CfgUfGfUfguaugcacs | 2758 | VPusAfsgugCfaUfAfcacaCfgUfgccugscsa | 2893 | UGCAGGCACGUGUGUAUGCA | 3028 |
| 1735118 | usa | | | | CUC | |
| AD- | asusggc(Ada)GfuCfAfAfcaaaggccsu | 2759 | VPusAfsggcCfuUfUfguugAfcUfgccauscs | 2894 | GGAUGGCAGUCAACAAAGGC | 3029 |
| 1735119 | sa | | c | | CUC | |
| AD- | csasaac(Uda)UfgUfGfGfccaaaagasu | 2760 | VPusAfsucuUfuUfGfgccaCfaAfguuugsgs | 2895 | ACCAAACUUGUGGCCAAAAG | 3030 |
| 1735120 | sa | | u | | AUA | |
| AD- | uscsuga(Cda)CfaAfCfAfcccucucusc | 2761 | VPusGfsagaGfaGfGfguguUfgGfucagasgs | 2896 | GCUCUGACCAACACCCUCUCU | 3031 |
| 1735121 | sa | | c | | CG | |
| AD- | uscsaca(Uda)UfaUfAfAfaucccacusc | 2762 | VPusGfsaguGfgGfAfuuuaUfaAfugugasus | 2897 | UAUCACAUUAUAAAUCCCAC | 3032 |
| 1735122 | sa | | a | | UCG | |
| AD- | gsasgga(Gda)AfaCfAfAfguucagugs | 2763 | VPusUfscacUfgAfAfcuugUfuCfuccucsas | 2898 | CUGAGGAGAACAAGUUCAGU | 3033 |
| 1735123 | asa | | g | | GAG | |
| AD- | gsasgca(Gda)GfaGfAfAfugugcugcs | 2764 | VPusUfsgcaGfcAfCfauucUfcCfugcucsasg | 2899 | CUGAGCAGGAGAAUGUGCUG | 3034 |
| 1735124 | asa | | | | CAG | |
| AD- | gsasaga(Cda)CfuAfCfAfccagcacusa | 2765 | VPusUfsaguGfcUfGfguguAfgGfucuucsus | 2900 | CAGAAGACCUACACCAGCACU | 3035 |
| 1735125 | sa | | g | | AA | |
| AD- | asgsgcc(Uda)CfaCfCfUfugcuggausg | 2766 | VPusCfsaucCfaGfCfaaggUfgAfggccususu | 2901 | AAAGGCCUCACCUUGCUGGA | 3036 |
| 1735126 | sa | | | | UGG | |
| AD- | asgscca(Gda)CfuGfCfUfugcucacasg | 2767 | VPusCfsuguGfaGfCfaagcAfgCfuggcuscs | 2902 | AGAGCCAGCUGCUUGCUCACA | 3037 |
| 1735127 | sa | | u | | GC | |
| AD- | gsgsgcu(Cda)UfgUfGfAfgacugaggs | 2768 | VPusAfsccuCfaGfUfcucaCfaGfagcccsasg | 2903 | CUGGGCUCUGUGAGACUGAG | 3038 |
| 1735128 | usa | | | | GUG | |
| AD- | csgsaga(Gda)CfgAfAfUfuccgggacsa | 2769 | VPusUfsgucCfcGfGfaauuCfgCfucucgsgs | 2904 | CCCGAGAGCGAAUUCCGGGAC | 3039 |
| 1735129 | sa | | g | | AU | |
| AD- | gsusggc(Ada)CfaUfAfCfccugcagasa | 2770 | VPusUfsucuGfcAfGfgguaUfgUfgccacsas | 2905 | GUGUGGCACAUACCCUGCAG | 3040 |
| 1735130 | sa | | c | | AAG | |
| AD- | gsusgga(Cda)AfcAfGfUfggugcauus | 2771 | VPusUfsaauGfcAfCfcacuGfuGfuccacscsa | 2906 | UGGUGGACACAGUGGUGCAU | 3041 |
| 1735131 | asa | | | | UAC | |
| AD- | asasgcc(Cda)AfgAfAfGfucucccaasg | 2772 | VPusCfsuugGfgAfGfacuuCfuGfggcuusgs | 2907 | GCAAGCCCAGAAGUCUCCCAA | 3042 |
| 1735132 | sa | | c | | GG | |
| AD- | gsgscca(Uda)GfuCfCfCfuaucagausg | 2773 | VPusCfsaucUfgAfUfagggAfcAfuggccscs | 2908 | AGGGCCAUGUCCCUAUCAGA | 3043 |
| 1735133 | sa | | u | | UGC | |
| AD- | gscscgg(Cda)GfgGfUfUfucucuaacsa | 2774 | VPusUfsguuAfgAfGfaaacCfcGfccggcscsc | 2909 | GGGCCGGCGGGUUUCUCUAA | 3044 |
| 1735134 | sa | | | | CAA | |
| AD- | csusgaa(Gda)GfaCfAfCfcaucuccasc | 2775 | VPusGfsuggAfgAfUfggugUfcCfuucagscs | 2910 | AGCUGAAGGACACCAUCUCCA | 3045 |
| 1735135 | sa | | u | | CC | |
| AD- | gsascca(Cda)CfaUfCfUfcggcugugsa | 2776 | VPusUfscacAfgCfCfgagaUfgGfuggucsus | 2911 | CAGACCACCAUCUCGGCUGUG | 3046 |
| 1735136 | sa | | g | | AC | |
| AD- | cscsagu(Uda)UfuUfAfAfgggacaccsa | 2777 | VPusUfsgguGfuCfCfcuuaAfaAfacuggscs | 2912 | AGCCAGUUUUUAAGGGACAC | 3047 |
| 1735137 | sa | | u | | CAG | |
| AD- | gsgscca(Gda)AfgAfCfAfcugcggaas | 2778 | VPusAfsuucCfgCfAfguguCfuCfuggccsas | 2913 | GUGGCCAGAGACACUGCGGA | 3048 |
| 1735138 | usa | | c | | AUU | |
| AD- | csgsuuc(Cda)CfaUfCfGfcgagcacusu | 2779 | VPusAfsaguGfcUfCfgcgaUfgGfgaacgscs | 2914 | AGCGUUCCCAUCGCGAGCACU | 3049 |
| 1735139 | sa | | u | | UC | |
| AD- | cscsggg(Ada)CfaUfCfGfacaacccasc | 2780 | VPusGfsuggGfuUfGfucgaUfgUfcccggsas | 2915 | UUCCGGGACAUCGACAACCCA | 3050 |
| 1735140 | sa | | a | | CC | |
| AD- | uscsuga(Uda)GfaUfCfUfaggcucccsa | 2781 | VPusUfsgggAfgCfCfuagaUfcAfucagasgs | 2916 | CCUCUGAUGAUCUAGGCUCCC | 3051 |
| 1735141 | sa | | g | | AG | |
| AD- | csusgca(Gda)AfaGfAfCfccuccagasc | 2782 | VPusGfsucuGfgAfGfggucUfuCfugcagsgs | 2917 | CCCUGCAGAAGACCCUCCAGA | 3052 |
| 1735142 | sa | | g | | CC | |
| AD- | csgscca(Gda)UfaGfCfUfuggcugccsu | 2783 | VPusAfsggcAfgCfCfaagcUfaCfuggcgscsu | 2918 | AGCGCCAGUAGCUUGGCUGCC | 3053 |
| 1735143 | sa | | | | UG | |
| AD- | csgscuu(Cda)AfcAfGfGfcugaguccsa | 2784 | VPusUfsggaCfuCfAfgccuGfuGfaagcgsgs | 2919 | GCCGCUUCACAGGCUGAGUCC | 3054 |
| 1735144 | sa | | c | | AG | |
| AD- | ususuuu(Uda)UfaGfCfAfuccuuuugs | 2785 | VPusCfscaaAfaGfGfaugcUfaAfaaaaasasa | 2920 | UUUUUUUUUAGCAUCCUUUU | 3055 |
| 1735145 | gsa | | | | GGG | |
| AD- | gsasagc(Cda)AfaAfGfCfgcagggucsa | 2786 | VPusUfsgacCfcUfGfcgcuUfuGfgcuucsus | 2921 | GAGAAGCCAAAGCGCAGGGU | 3056 |
| 1735146 | sa | | c | | CAG | |
| AD- | gsasgga(Gda)GfaUfCfAfugaggaccsa | 2787 | VPusUfsgguCfcUfCfaugaUfcCfuccucscsu | 2922 | AGGAGGAGGAUCAUGAGGAC | 3057 |
| 1735147 | sa | | | | CAG | |
| AD- | gscsacg(Cda)AfuUfAfCfagccagcusg | 2788 | VPusCfsagcUfgGfCfuguaAfuGfcgugcsgs | 2923 | CCGCACGCAUUACAGCCAGCU | 3058 |
| 1735148 | sa | | g | | GC | |
| AD- | csusuca(Uda)UfgAfAfAfaccaccacsg | 2789 | VPusCfsgugGfuGfGfuuuuCfaAfugaagsgs | 2924 | CCCUUCAUUGAAAACCACCAC | 3059 |
| 1735149 | sa | | g | | GG | |
| AD- | ususuuu(Gda)AfuGfGfCfcacauaaus | 2790 | VPusUfsauuAfuGfUfggccAfuCfaaaaasgsa | 2925 | UCUUUUUGAUGGCCACAUAA | 3060 |
| 1735150 | asa | | | | UAA | |
| AD- | cscsacc(Uda)GfgAfGfGfaaaagaucsc | 2791 | VPusGfsgauCfuUfUfuccuCfcAfgguggsus | 2926 | GACCACCUGGAGGAAAAGAU | 3061 |
| 1735151 | sa | | c | | CCC | |
| AD- | csasagg(Cda)CfaAfGfCfcaagccucsu | 2792 | VPusAfsgagGfcUfUfggcuUfgGfccuugsgs | 2927 | CCCAAGGCCAAGCCAAGCCUC | 3062 |
| 1735152 | sa | | g | | UU | |
| AD- | gsasgga(Cda)CfaGfAfCfagacacggsa | 2793 | VPusUfsccgUfgUfCfugucUfgGfuccucsas | 2928 | AUGAGGACCAGACAGACACG | 3063 |
| 1735153 | sa | | u | | GAG | |
| AD- | gsasugc(Cda)CfuGfAfAfgggcguuas | 2794 | VPusGfsuaaCfgCfCfcuucAfgGfgcaucsusg | 2929 | CAGAUGCCCUGAAGGGCGUU | 3064 |
| 1735154 | csa | | | | ACU | |
| AD- | cscsaac(Ada)CfuCfGfAfgcuggccgsa | 2795 | VPusUfscggCfcAfGfcucgAfgUfguuggscs | 2930 | UGCCAACACUCGAGCUGGCCG | 3065 |
| 1735155 | sa | | a | | AC | |
| AD- | csasggg(Cda)UfaUfGfCfaccugcagsg | 2796 | VPusCfscugCfaGfGfugcaUfaGfcccugscsa | 2931 | UGCAGGGCUAUGCACCUGCA | 3066 |
| 1735156 | sa | | | | GGC | |
| AD- | asasggu(Gda)GfuGfGfAfguaccuccs | 2797 | VPusAfsggaGfgUfAfcuccAfcCfaccuuscsu | 2932 | AGAAGGUGGUGGAGUACCUC | 3067 |
| 1735157 | usa | | | | CUC | |
| AD- | ususgaa(Gda)CfuUfGfAfggagcgags | 2798 | VPusCfscucGfcUfCfcucaAfgCfuucaascsu | 2933 | AGUUGAAGCUUGAGGAGCGA | 3068 |
| 1735158 | gsa | | | | GGA | |
| AD- | csgsugg(Cda)CfaUfGfUfggaucccasg | 2799 | VPusCfsuggGfaUfCfcacaUfgGfccacgsgsu | 2934 | ACCGUGGCCAUGUGGAUCCCA | 3069 |
| 1735159 | sa | | | | GG | |
| AD- | csasgca(Cda)UfaAfGfGfaagcccacsc | 2800 | VPusGfsgugGfgCfUfuccuUfaGfugcugsgs | 2935 | ACCAGCACUAAGGAAGCCCAC | 3070 |
| 1735160 | sa | | u | | CC | |
| AD- | gsuscgc(Uda)GfaUfGfGfagcccgagsa | 2801 | VPusUfscucGfgGfCfuccaUfcAfgcgacsasg | 2936 | CUGUCGCUGAUGGAGCCCGA | 3071 |
| 1735161 | sa | | | | GAG | |
| AD- | ususcuu(Cda)CfgGfCfCfcagcgucasu | 2802 | VPusAfsugaCfgCfUfgggcCfgGfaagaasgsc | 2937 | GCUUCUUCCGGCCCAGCGUCA | 3072 |
| 1735162 | sa | | | | UG | |
| AD- | csusgcu(Cda)CfuGfGfAfcaccagcasa | 2803 | VPusUfsugcUfgGfUfguccAfgGfagcagsgs | 2938 | CCCUGCUCCUGGACACCAGCA | 3073 |
| 1735163 | sa | | g | | AG | |
| AD- | ususucu(Gda)AfgUfAfCfccgccgcus | 2804 | VPusAfsagcGfgCfGfgguaCfuCfagaaasgsu | 2939 | ACUUUCUGAGUACCCGCCGCU | 3074 |
| 1735164 | usa | | | | UC | |
| AD- | csgscag(Gda)CfuGfUfCfcacccagusu | 2805 | VPusAfsacuGfgGfUfggacAfgCfcugcgsgs | 2940 | UCCGCAGGCUGUCCACCCAGU | 3075 |
| 1735165 | sa | | a | | UC | |
| AD- | gsgscug(Uda)GfaCfAfUfgggcaccus | 2806 | VPusCfsaggUfgCfCfcaugUfcAfcagccsgsa | 2941 | UCGGCUGUGACAUGGGCACC | 3076 |
| 1735166 | gsa | | | | UGC | |
| AD- | uscsagu(Gda)AfgGfUfAfgcagcccus | 2807 | VPusCfsaggGfcUfGfcuacCfuCfacugasasc | 2942 | GUUCAGUGAGGUAGCAGCCC | 3077 |
| 1735167 | gsa | | | | UGC | |
| AD- | asgscgu(Cda)AfuGfGfAfgcccauccsu | 2808 | VPusAfsggaUfgGfGfcuccAfuGfacgcusgs | 2943 | CCAGCGUCAUGGAGCCCAUCC | 3078 |
| 1735168 | sa | | g | | UG | |
| AD- | csusgga(Uda)GfgAfGfAfccucccugs | 2809 | VPusUfscagGfgAfGfgucuCfcAfuccagscsa | 2944 | UGCUGGAUGGAGACCUCCCU | 3079 |
| 1735169 | asa | | | | GAG | |
| AD- | uscsagc(Cda)GfgAfGfUfgaguguugs | 2810 | VPusCfscaaCfaCfUfcacuCfcGfgcugascsc | 2945 | GGUCAGCCGGAGUGAGUGUU | 3080 |
| 1735170 | gsa | | | | GGG | |
| AD- | csusggc(Cda)GfaCfUfGfgcuucuggs | 2811 | VPusUfsccaGfaAfGfccagUfcGfgccagscsu | 2946 | AGCUGGCCGACUGGCUUCUG | 3081 |
| 1735171 | asa | | | | GAG | |
| AD- | gsgsugc(Uda)GfcAfCfCfucacaccasg | 2812 | VPusCfsuggUfgUfGfagguGfcAfgcaccscs | 2947 | AGGGUGCUGCACCUCACACCA | 3082 |
| 1735172 | sa | | u | | GC | |
| AD- | gsgscug(Cda)CfuGfGfAfgcauggags | 2813 | VPusGfscucCfaUfGfcuccAfgGfcagccsasa | 2948 | UUGGCUGCCUGGAGCAUGGA | 3083 |
| 1735173 | csa | | | | GCC | |
| AD- | cscsugc(Ada)GfcUfGfUfgcugggcas | 2814 | VPusAfsugcCfcAfGfcacaGfcUfgcaggsusg | 2949 | CACCUGCAGCUGUGCUGGGCA | 3084 |
| 1735174 | usa | | | | UG | |
| AD- | asgsugg(Cda)CfaUfGfCfaggcggugs | 2815 | VPusAfscacCfgCfCfugcaUfgGfccacusgsa | 2950 | UCAGUGGCCAUGCAGGCGGU | 3085 |
| 1735175 | usa | | | | GUC | |
| AD- | gsascuu(Gda)GfcCfUfUfgggcagcas | 2816 | VPusAfsugcUfgCfCfcaagGfcCfaagucsgsg | 2951 | CCGACUUGGCCUUGGGCAGCA | 3086 |
| 1735176 | usa | | | | UU | |
| AD- | gsasgcg(Ada)AfgUfGfCfggguacccs | 2817 | VPusAfsgggUfaCfCfcgcaCfuUfcgcucscsu | 2952 | AGGAGCGAAGUGCGGGUACC | 3087 |
| 1735177 | usa | | | | CUG | |
| AD- | csusggu(Gda)GfcCfUfCfugugugcas | 2818 | VPusUfsugcAfcAfCfagagGfcCfaccagsgsg | 2953 | CCCUGGUGGCCUCUGUGUGCA | 3088 |
| 1735178 | asa | | | | AU | |
| AD- | asasccc(Ada)CfcAfGfCfcgaggucgsa | 2819 | VPusUfscgaCfcUfCfggcuGfgUfggguusgs | 2954 | ACAACCCACCAGCCGAGGUCG | 3089 |
| 1735179 | sa | | u | | AG | |
| AD- | usgsaug(Cda)UfgCfCfAfaggcgcacsu | 2820 | VPusAfsgugCfgCfCfuuggCfaGfcaucasusc | 2955 | GAUGAUGCUGCCAAGGCGCA | 3090 |
| 1735180 | sa | | | | CUU | |
| AD- | cscsaau(Gda)AfgCfUfGfgccugccgsa | 2821 | VPusUfscggCfaGfGfccagCfuCfauuggscsa | 2956 | UGCCAAUGAGCUGGCCUGCCG | 3091 |
| 1735181 | sa | | | | AG | |
| AD- | gsusgcu(Gda)CfaGfCfGfgguccugcs | 2822 | VPusUfsgcaGfgAfCfccgcUfgCfagcacsasu | 2957 | AUGUGCUGCAGCGGGUCCUG | 3092 |
| 1735182 | asa | | | | CAG | |
| AD- | gsgsgcg(Uda)GfcAfGfAfgcgccagus | 2823 | VPusUfsacuGfgCfGfcucuGfcAfcgcccsusu | 2958 | AAGGGCGUGCAGAGCGCCAG | 3093 |
| 1735183 | asa | | | | UAG | |
| AD- | gsgsuga(Gda)UfgGfCfAfccugcgaas | 2824 | VPusAfsuucGfcAfGfgugcCfaCfucaccsasc | 2959 | GUGGUGAGUGGCACCUGCGA | 3094 |
| 1735184 | usa | | | | AUG | |
| AD- | ascsgga(Gda)GfgAfGfAfggacacggs | 2825 | VPusUfsccgUfgUfCfcucuCfcCfuccgusgsu | 2960 | ACACGGAGGGAGAGGACACG | 3095 |
| 1735185 | asa | | | | GAG | |

TABLE 10
Unmodified Sense and Antisense Strand Sequences of PLIN1 dsRNA Agents Comprising a GalNAc
Derivative Targeting Ligand

| | | SEQ | Range in | | SEQ | Range in |
|---|---|---|---|---|---|---|
| Duplex | Sense Sequence | ID | NM_ | Antisense Sequence | ID | NM_ |
| Name | 5′ to 3′ | NO: | 002666.5 | 5′ to 3′ | NO: | 002666.5 |
| AD-1735186 | CAUCUCUUUAACCAAACUUGA | 2421 | 1995-2015 | UCAAGUUUGGUUAAAGAGAUGA | 2556 | 1993-2015 |
| | | | | A | | |
| AD-1735187 | CUCUAACAAAUAAACAGAACA | 2422 | 1740-1760 | UGUUCUGUUUAUUUGUUAGAGA | 2557 | 1738-1760 |
| | | | | A | | |
| AD-1735188 | UGCAUAGUCACUCUUUUGAUA | 2423 | 2390-2410 | UAUCAAAAGAGUGACUAUGCAG | 2558 | 2388-2410 |
| | | | | G | | |
| AD-1735189 | AACUUGAUUUUUCAUCUCUUA | 2424 | 1983-2003 | UAAGAGAUGAAAAAUCAAGUUA | 2559 | 1981-2003 |
| | | | | G | | |
| AD-1735190 | GACACAUUCUUAGCACUGAAA | 2425 | 2128-2148 | UUUCAGUGCUAAGAAUGUGUCA | 2560 | 2126-2148 |
| | | | | A | | |
| AD-1735191 | ACGCCUUAUUUGAUUUAACUA | 2426 | 2807-2827 | UAGUUAAAUCAAAUAAGGCGUA | 2561 | 2805-2827 |
| | | | | U | | |
| AD-1735192 | UAGUCUUCGAAAUGUUAAUAA | 2427 | 2474-2494 | UUAUUAACAUUUCGAAGACUAG | 2562 | 2472-2494 |
| | | | | G | | |
| AD-1735193 | GAUGGACUUUUAAGUUGUUUA | 2428 | 2838-2858 | UAAACAACUUAAAAGUCCAUCA | 2563 | 2836-2858 |
| | | | | U | | |
| AD-1735194 | UGCUUUUUUCACUUAAUAAUA | 2429 | 2702-2722 | UAUUAUUAAGUGAAAAAAGCAG | 2564 | 2700-2722 |
| | | | | G | | |
| AD-1735195 | ACAUAAUAACUACUGCAUAAA | 2430 | 2780-2800 | UUUAUGCAGUAGUUAUUAUGUG | 2565 | 2778-2800 |
| | | | | G | | |
| AD-1735196 | UACUAGUGUCACUUUCUGAGA | 2431 | 2271-2291 | UCUCAGAAAGUGACACUAGUAU | 2566 | 2269-2291 |
| | | | | U | | |
| AD-1735197 | CAAUCAGAUGCAAAAGCUCUA | 2432 | 2234-2254 | UAGAGCUUUUGCAUCUGAUUGU | 2567 | 2232-2254 |
| | | | | U | | |
| AD-1735198 | UAAGAGUAAUUGCCUAACUUA | 2433 | 1968-1988 | UAAGUUAGGCAAUUACUCUUAU | 2568 | 1966-1988 |
| | | | | A | | |
| AD-1735199 | GGCUUGUUUAUGAACAUUAAA | 2434 | 82-102 | UUUAAUGUUCAUAAACAAGCCA | 2569 | 80-102 |
| | | | | U | | |
| AD-1735200 | UUUUUGCUACUGCAAACGAUA | 2435 | 2871-2891 | UAUCGUUUGCAGUAGCAAAAAA | 2570 | 2869-2891 |
| | | | | G | | |
| AD-1735201 | AACGAUGCUAUAAUAAAUGUA | 2436 | 2885-2905 | UACAUUUAUUAUAGCAUCGUUU | 2571 | 2883-2905 |
| | | | | G | | |
| AD-1735202 | UGGUUGUUACUAUAAGAGUAA | 2437 | 1956-1976 | UUACUCUUAUAGUAACAACCAA | 2572 | 1954-1976 |
| | | | | G | | |
| AD-1735203 | CUGAUGAACAUCCUCUGAUGA | 2438 | 2181-2201 | UCAUCAGAGGAUGUUCAUCAGA | 2573 | 2179-2201 |
| | | | | G | | |
| AD-1735204 | CUGCGGAUAAAUAUUUGCCAA | 2439 | 2059-2079 | UUGGCAAAUAUUUAUCCGCAGA | 2574 | 2057-2079 |
| | | | | G | | |
| AD-1735205 | CCAAAAUUCAGAUUCUGCCUA | 2440 | 2039-2059 | UAGGCAGAAUCUGAAUUUUGGA | 2575 | 2037-2059 |
| | | | | A | | |
| AD-1735206 | AAGGAUGUGUGUCUUUCUCCA | 2441 | 2547-2567 | UGGAGAAAGACACACAUCCUUU | 2576 | 2545-2567 |
| | | | | U | | |
| AD-1735207 | UGCGAAUGCUUCCAGAAGACA | 2442 | 231-251 | UGUCUUCUGGAAGCAUUCGCAG | 2577 | 229-251 |
| | | | | G | | |
| AD-1735208 | CGAAUGAGUAACUCCUGUCAA | 2443 | 2079-2099 | UUGACAGGAGUUACUCAUUCGU | 2578 | 2077-2099 |
| | | | | G | | |
| AD-1735209 | UGGGUGUUUAUUUAAAAUACA | 2444 | 2254-2274 | UGUAUUUUAAAUAAACACCCAA | 2579 | 2252-2274 |
| | | | | G | | |
| AD-1735210 | CAAAAGAUAUUUGACCGUUUA | 2445 | 2019-2039 | UAAACGGUCAAAUAUCUUUUGG | 2580 | 2017-2039 |
| | | | | C | | |
| AD-1735211 | AUACAGCUUGGAGAGAUUUUA | 2446 | 2720-2740 | UAAAAUCUCUCCAAGCUGUAUU | 2581 | 2718-2740 |
| | | | | A | | |
| AD-1735212 | AAAUGCAUUCAUACAAUUACA | 2447 | 2509-2529 | UGUAAUUGUAUGAAUGCAUUUU | 2582 | 2507-2529 |
| | | | | C | | |
| AD-1735213 | UCCCACUCGCUCUUUUUGAUA | 2448 | 2756-2776 | UAUCAAAAAGAGCGAGUGGGAU | 2583 | 2754-2776 |
| | | | | U | | |
| AD-1735214 | GCUUGUGUUUACACAAGCUGA | 2449 | 2664-2684 | UCAGCUUGUGUAAACACAAGCG | 2584 | 2662-2684 |
| | | | | G | | |
| AD-1735215 | CCUGCCUUACAUGGCUUGUUA | 2450 | 70-90 | UAACAAGCCAUGUAAGGCAGGU | 2585 | 68-90 |
| | | | | G | | |
| AD-1735216 | GAGAUUUUUGUAUCACAUUAA | 2451 | 2732-2752 | UUAAUGUGAUACAAAAAUCUCU | 2586 | 2730-2752 |
| | | | | C | | |
| AD-1735217 | AGCACUUCAGACAAGGUCCUA | 2452 | 540-560 | UAGGACCUUGUCUGAAGUGCUC | 2587 | 538-560 |
| | | | | G | | |
| AD-1735218 | UCACUCUCAGAGCCAGUUUUA | 2453 | 1895-1915 | UAAAACUGGCUCUGAGAGUGAA | 2588 | 1893-1915 |
| | | | | G | | |
| AD-1735219 | ACUGAACUCCUCUGUGAUCUA | 2454 | 2142-2162 | UAGAUCACAGAGGAGUUCAGUG | 2589 | 2140-2162 |
| | | | | C | | |
| AD-1735220 | UAGUUCCCUAAUGAUGGACUA | 2455 | 2826-2846 | UAGUCCAUCAUUAGGGAACUAG | 2590 | 2824-2846 |
| | | | | U | | |
| AD-1735221 | GGCGUUACUGACAACGUGGUA | 2456 | 1296-1316 | UACCACGUUGUCAGUAACGCCC | 2591 | 1294-1316 |
| | | | | U | | |
| AD-1735222 | UGAUCUAGGAUGAUCUGUUCA | 2457 | 2156-2176 | UGAACAGAUCAUCCUAGAUCAC | 2592 | 2154-2176 |
| | | | | A | | |
| AD-1735223 | GUGAUGAUAUAUAGACUUUAA | 2458 | 2427-2447 | UUAAAGUCUAUAUAUCAUCACC | 2593 | 2425-2447 |
| | | | | A | | |
| AD-1735224 | GGCUACUUUGAAGGGAACAAA | 2459 | 2217-2237 | UUUGUUCCCUUCAAAGUAGCCU | 2594 | 2215-2237 |
| | | | | G | | |
| AD-1735225 | CUGCUCUGAUUCUAUGGCUUA | 2460 | 1937-1957 | UAAGCCAUAGAAUCAGAGCAGG | 2595 | 1935-1957 |
| | | | | C | | |
| AD-1735226 | AGAUUGCUUCUGAGCUGAAGA | 2461 | 463-483 | UCUUCAGCUCAGAAGCAAUCUU | 2596 | 461-483 |
| | | | | U | | |
| AD-1735227 | GCCAUCUCUUGUGUAUGCAGA | 2462 | 2607-2627 | UCUGCAUACACAAGAGAUGGCA | 2597 | 2605-2627 |
| | | | | C | | |
| AD-1735228 | AGCCACAUUUCCAUUUGCAUA | 2463 | 2354-2374 | UAUGCAAAUGGAAAUGUGGCUG | 2598 | 2352-2374 |
| | | | | U | | |
| AD-1735229 | UCCAGACAGAAGGUUUUGACA | 2464 | 2111-2131 | UGUCAAAACCUUCUGUCUGGAC | 2599 | 2109-2131 |
| | | | | C | | |
| AD-1735230 | CUCUCGAUACACCGUGCAGAA | 2465 | 818-838 | UUCUGCACGGUGUAUCGAGAGA | 2600 | 816-838 |
| | | | | G | | |
| AD-1735231 | GGCGCACUUUUUAUUUUUAUA | 2466 | 1838-1858 | UAUAAAAAUAAAAAGUGCGCCU | 2601 | 1836-1858 |
| | | | | U | | |
| AD-1735232 | GAGCGUUGCCACUUUCAAAGA | 2467 | 1775-1795 | UCUUUGAAAGUGGCAACGCUCG | 2602 | 1773-1795 |
| | | | | C | | |
| AD-1735233 | UUGCAUCAUUACUGCCUUCAA | 2468 | 2368-2388 | UUGAAGGCAGUAAUGAUGCAAA | 2603 | 2366-2388 |
| | | | | U | | |
| AD-1735234 | GCGCAAGAAGAGCUGAGUCGA | 2469 | 1685-1705 | UCGACUCAGCUCUUCUUGCGCA | 2604 | 1683-1705 |
| | | | | G | | |
| AD-1735235 | UGCGGAAUUUGCUGCCAACAA | 2470 | 611-631 | UUGUUGGCAGCAAAUUCCGCAG | 2605 | 609-631 |
| | | | | U | | |
| AD-1735236 | GUGUGCAAUGCCUAUGAGAAA | 2471 | 291-311 | UUUCUCAUAGGCAUUGCACACA | 2606 | 289-311 |
| | | | | G | | |
| AD-1735237 | GAAUUCAAAUAUUGCAAAAGA | 2472 | 2530-2550 | UCUUUUGCAAUAUUUGAAUUCU | 2607 | 2528-2550 |
| | | | | G | | |
| AD-1735238 | CCUGUGUGCUUUGUAGAGCCA | 2473 | 2320-2340 | UGGCUCUACAAAGCACACAGGC | 2608 | 2318-2340 |
| | | | | C | | |
| AD-1735239 | UCCAGACAAGGAAGAGUCAGA | 2474 | 710-730 | UCUGACUCUUCCUUGUCUGGAG | 2609 | 708-730 |
| | | | | G | | |
| AD-1735240 | UGCCAGAAACAGCAUCAGCGA | 2475 | 509-529 | UCGCUGAUGCUGUUUCUGGCAC | 2610 | 507-529 |
| | | | | U | | |
| AD-1735241 | GCAUAAUAUGGAUACGCCUUA | 2476 | 2794-2814 | UAAGGCGUAUCCAUAUUAUGCA | 2611 | 2792-2814 |
| | | | | G | | |
| AD-1735242 | AGCCUCAUCCUGAUGAUGCUA | 2477 | 1813-1833 | UAGCAUCAUCAGGAUGAGGCUG | 2612 | 1811-1833 |
| | | | | A | | |
| AD-1735243 | AAGCCUCUUGAGCAGGGUUGA | 2478 | 779-799 | UCAACCCUGCUCAAGAGGCUUG | 2613 | 777-799 |
| | | | | G | | |
| AD-1735244 | UAAAGGGAAGAAGUUGAAGCA | 2479 | 99-119 | UGCUUCAACUUCUUCCCUUUAA | 2614 | 97-119 |
| | | | | U | | |
| AD-1735245 | GGCAGCAUUGAGAAGGUGGUA | 2480 | 675-695 | UACCACCUUCUCAAUGCUGCCC | 2615 | 673-695 |
| | | | | A | | |
| AD-1735246 | GUCAGCGACAGCUUCUUCCGA | 2481 | 1617-1637 | UCGGAAGAAGCUGUCGCUGACC | 2616 | 1615-1637 |
| | | | | C | | |
| AD-1735247 | CUGCUGUCUCCUCAACCAAGA | 2482 | 1243-1263 | UCUUGGUUGAGGAGACAGCAGG | 2617 | 1241-1263 |
| | | | | G | | |
| AD-1735248 | AGGAGGAAGAAUUGGAGACUA | 2483 | 1054-1074 | UAGUCUCCAAUUCUUCCUCCUC | 2618 | 1052-1074 |
| | | | | C | | |
| AD-1735249 | CCUGUCACCACUCUGAAGGUA | 2484 | 2092-2112 | UACCUUCAGAGUGGUGACAGGA | 2619 | 2090-2112 |
| | | | | G | | |
| AD-1735250 | AGACUUUAUGUAUAGCCACAA | 2485 | 2439-2459 | UUGUGGCUAUACAUAAAGUCUA | 2620 | 2437-2459 |
| | | | | U | | |
| AD-1735251 | GUUACGCAUGCCUGCUUUUUA | 2486 | 2690-2710 | UAAAAAGCAGGCAUGCGUAACA | 2621 | 2688-2710 |
| | | | | C | | |
| AD-1735252 | CCAGUUCACAGCUGCCAAUGA | 2487 | 377-397 | UCAUUGGCAGCUGUGAACUGGG | 2622 | 375-397 |
| | | | | U | | |
| AD-1735253 | CAGGCACGUGUGUAUGCACUA | 2488 | 2640-2660 | UAGUGCAUACACACGUGCCUGC | 2623 | 2638-2660 |
| | | | | A | | |
| AD-1735254 | AUGGCAGUCAACAAAGGCCUA | 2489 | 132-152 | UAGGCCUUUGUUGACUGCCAUC | 2624 | 130-152 |
| | | | | C | | |
| AD-1735255 | CAAACUUGUGGCCAAAAGAUA | 2490 | 2007-2027 | UAUCUUUUGGCCACAAGUUUGG | 2625 | 2005-2027 |
| | | | | U | | |
| AD-1735256 | UCUGACCAACACCCUCUCUCA | 2491 | 803-823 | UGAGAGAGGGUGUUGGUCAGAG | 2626 | 801-823 |
| | | | | C | | |
| AD-1735257 | UCACAUUAUAAAUCCCACUCA | 2492 | 2744-2764 | UGAGUGGGAUUUAUAAUGUGAU | 2627 | 2742-2764 |
| | | | | A | | |
| AD-1735258 | GAGGAGAACAAGUUCAGUGAA | 2493 | 1074-1094 | UUCACUGAACUUGUUCUCCUCA | 2628 | 1072-1094 |
| | | | | G | | |
| AD-1735259 | GAGCAGGAGAAUGUGCUGCAA | 2494 | 177-197 | UUGCAGCACAUUCUCCUGCUCA | 2629 | 175-197 |
| | | | | G | | |
| AD-1735260 | GAAGACCUACACCAGCACUAA | 2495 | 245-265 | UUAGUGCUGGUGUAGGUCUUCU | 2630 | 243-265 |
| | | | | G | | |
| AD-1735261 | AGGCCUCACCUUGCUGGAUGA | 2496 | 146-166 | UCAUCCAGCAAGGUGAGGCCUU | 2631 | 144-166 |
| | | | | U | | |
| AD-1735262 | AGCCAGCUGCUUGCUCACAGA | 2497 | 2336-2356 | UCUGUGAGCAAGCAGCUGGCUC | 2632 | 2334-2356 |
| | | | | U | | |
| AD-1735263 | GGGCUCUGUGAGACUGAGGUA | 2498 | 13-33 | UACCUCAGUCUCACAGAGCCCA | 2633 | 11-33 |
| | | | | G | | |
| AD-1735264 | CGAGAGCGAAUUCCGGGACAA | 2499 | 1367-1387 | UUGUCCCGGAAUUCGCUCUCGG | 2634 | 1365-1387 |
| | | | | G | | |
| AD-1735265 | GUGGCACAUACCCUGCAGAAA | 2500 | 1134-1154 | UUUCUGCAGGGUAUGUGCCACA | 2635 | 1132-1154 |
| | | | | C | | |
| AD-1735266 | GUGGACACAGUGGUGCAUUAA | 2501 | 1314-1334 | UUAAUGCACCACUGUGUCCACC | 2636 | 1312-1334 |
| | | | | A | | |
| AD-1735267 | AAGCCCAGAAGUCUCCCAAGA | 2502 | 751-771 | UCUUGGGAGACUUCUGGGCUUG | 2637 | 749-771 |
| | | | | C | | |
| AD-1735268 | GGCCAUGUCCCUAUCAGAUGA | 2503 | 1268-1288 | UCAUCUGAUAGGGACAUGGCCC | 2638 | 1266-1288 |
| | | | | U | | |
| AD-1735269 | GCCGGCGGGUUUCUCUAACAA | 2504 | 1728-1748 | UUGUUAGAGAAACCCGCCGGCC | 2639 | 1726-1748 |
| | | | | C | | |
| AD-1735270 | CUGAAGGACACCAUCUCCACA | 2505 | 477-497 | UGUGGAGAUGGUGUCCUUCAGC | 2640 | 475-497 |
| | | | | U | | |
| AD-1735271 | GACCACCAUCUCGGCUGUGAA | 2506 | 1163-1183 | UUCACAGCCGAGAUGGUGGUCU | 2641 | 1161-1183 |
| | | | | G | | |
| AD-1735272 | CCAGUUUUUAAGGGACACCAA | 2507 | 1907-1927 | UUGGUGUCCCUUAAAAACUGGC | 2642 | 1905-1927 |
| | | | | U | | |
| AD-1735273 | GGCCAGAGACACUGCGGAAUA | 2508 | 599-619 | UAUUCCGCAGUGUCUCUGGCCA | 2643 | 597-619 |
| | | | | C | | |
| AD-1735274 | CGUUCCCAUCGCGAGCACUUA | 2509 | 527-547 | UAAGUGCUCGCGAUGGGAACGC | 2644 | 525-547 |
| | | | | U | | |
| AD-1735275 | CCGGGACAUCGACAACCCACA | 2510 | 1379-1399 | UGUGGGUUGUCGAUGUCCCGGA | 2645 | 1377-1399 |
| | | | | A | | |
| AD-1735276 | UCUGAUGAUCUAGGCUCCCAA | 2511 | 2194-2214 | UUGGGAGCCUAGAUCAUCAGAG | 2646 | 2192-2214 |
| | | | | G | | |
| AD-1735277 | CUGCAGAAGACCCUCCAGACA | 2512 | 1146-1166 | UGUCUGGAGGGUCUUCUGCAGG | 2647 | 1144-1166 |
| | | | | G | | |
| AD-1735278 | CGCCAGUAGCUUGGCUGCCUA | 2513 | 323-343 | UAGGCAGCCAAGCUACUGGCGC | 2648 | 321-343 |
| | | | | U | | |
| AD-1735279 | CGCUUCACAGGCUGAGUCCAA | 2514 | 2298-2318 | UUGGACUCAGCCUGUGAAGCGG | 2649 | 2296-2318 |
| | | | | C | | |
| AD-1735280 | UUUUUUUAGCAUCCUUUUGGA | 2515 | 1871-1891 | UCCAAAAGGAUGCUAAAAAAAA | 2650 | 1869-1891 |
| | | | | A | | |
| AD-1735281 | GAAGCCAAAGCGCAGGGUCAA | 2516 | 1601-1621 | UUGACCCUGCGCUUUGGCUUCU | 2651 | 1599-1621 |
| | | | | C | | |
| AD-1735282 | GAGGAGGAUCAUGAGGACCAA | 2517 | 1005-1025 | UUGGUCCUCAUGAUCCUCCUCC | 2652 | 1003-1025 |
| | | | | U | | |
| AD-1735283 | GCACGCAUUACAGCCAGCUGA | 2518 | 1666-1686 | UCAGCUGGCUGUAAUGCGUGCG | 2653 | 1664-1686 |
| | | | | G | | |
| AD-1735284 | CUUCAUUGAAAACCACCACGA | 2519 | 2585-2605 | UCGUGGUGGUUUUCAAUGAAGG | 2654 | 2583-2605 |
| | | | | G | | |
| AD-1735285 | UUUUUGAUGGCCACAUAAUAA | 2520 | 2768-2788 | UUAUUAUGUGGCCAUCAAAAAG | 2655 | 2766-2788 |
| | | | | A | | |
| AD-1735286 | CCACCUGGAGGAAAAGAUCCA | 2521 | 419-439 | UGGAUCUUUUCCUCCAGGUGGU | 2656 | 417-439 |
| | | | | C | | |
| AD-1735287 | CAAGGCCAAGCCAAGCCUCUA | 2522 | 767-787 | UAGAGGCUUGGCUUGGCCUUGG | 2657 | 765-787 |
| | | | | G | | |
| AD-1735288 | GAGGACCAGACAGACACGGAA | 2523 | 1017-1037 | UUCCGUGUCUGUCUGGUCCUCA | 2658 | 1015-1037 |
| | | | | U | | |
| AD-1735289 | GAUGCCCUGAAGGGCGUUACA | 2524 | 1284-1304 | UGUAACGCCCUUCAGGGCAUCU | 2659 | 1282-1304 |
| | | | | G | | |
| AD-1735290 | CCAACACUCGAGCUGGCCGAA | 2525 | 625-645 | UUCGGCCAGCUCGAGUGUUGGC | 2660 | 623-645 |
| | | | | A | | |
| AD-1735291 | CAGGGCUAUGCACCUGCAGGA | 2526 | 2624-2644 | UCCUGCAGGUGCAUAGCCCUGC | 2661 | 2622-2644 |
| | | | | A | | |
| AD-1735292 | AAGGUGGUGGAGUACCUCCUA | 2527 | 687-707 | UAGGAGGUACUCCACCACCUUC | 2662 | 685-707 |
| | | | | U | | |
| AD-1735293 | UUGAAGCUUGAGGAGCGAGGA | 2528 | 112-132 | UCCUCGCUCCUCAAGCUUCAAC | 2663 | 110-132 |
| | | | | U | | |
| AD-1735294 | CGUGGCCAUGUGGAUCCCAGA | 2529 | 869-889 | UCUGGGAUCCACAUGGCCACGG | 2664 | 867-889 |
| | | | | U | | |
| AD-1735295 | CAGCACUAAGGAAGCCCACCA | 2530 | 257-277 | UGGUGGGCUUCCUUAGUGCUGG | 2665 | 255-277 |
| | | | | U | | |
| AD-1735296 | GUCGCUGAUGGAGCCCGAGAA | 2531 | 1352-1372 | UUCUCGGGCUCCAUCAGCGACA | 2666 | 1350-1372 |
| | | | | G | | |
| AD-1735297 | UUCUUCCGGCCCAGCGUCAUA | 2532 | 1629-1649 | UAUGACGCUGGGCCGGAAGAAG | 2667 | 1627-1649 |
| | | | | C | | |
| AD-1735298 | CUGCUCCUGGACACCAGCAAA | 2533 | 733-753 | UUUGCUGGUGUCCAGGAGCAGG | 2668 | 731-753 |
| | | | | G | | |
| AD-1735299 | UUUCUGAGUACCCGCCGCUUA | 2534 | 2283-2303 | UAAGCGGCGGGUACUCAGAAAG | 2669 | 2281-2303 |
| | | | | U | | |
| AD-1735300 | CGCAGGCUGUCCACCCAGUUA | 2535 | 363-383 | UAACUGGGUGGACAGCCUGCGG | 2670 | 361-383 |
| | | | | A | | |
| AD-1735301 | GGCUGUGACAUGGGCACCUGA | 2536 | 1175-1195 | UCAGGUGCCCAUGUCACAGCCG | 2671 | 1173-1195 |
| | | | | A | | |
| AD-1735302 | UCAGUGAGGUAGCAGCCCUGA | 2537 | 1087-1107 | UCAGGGCUGCUACCUCACUGAA | 2672 | 1085-1107 |
| | | | | C | | |
| AD-1735303 | AGCGUCAUGGAGCCCAUCCUA | 2538 | 1641-1661 | UAGGAUGGGCUCCAUGACGCUG | 2673 | 1639-1661 |
| | | | | G | | |
| AD-1735304 | CUGGAUGGAGACCUCCCUGAA | 2539 | 159-179 | UUCAGGGAGGUCUCCAUCCAGC | 2674 | 157-179 |
| | | | | A | | |
| AD-1735305 | UCAGCCGGAGUGAGUGUUGGA | 2540 | 38-58 | UCCAACACUCACUCCGGCUGACC | 2675 | 36-58 |
| AD-1735306 | CUGGCCGACUGGCUUCUGGAA | 2541 | 637-657 | UUCCAGAAGCCAGUCGGCCAGC | 2676 | 635-657 |
| | | | | U | | |
| AD-1735307 | GGUGCUGCACCUCACACCAGA | 2542 | 1220-1240 | UCUGGUGUGAGGUGCAGCACCC | 2677 | 1218-1240 |
| | | | | U | | |
| AD-1735308 | GGCUGCCUGGAGCAUGGAGCA | 2543 | 335-355 | UGCUCCAUGCUCCAGGCAGCCA | 2678 | 333-355 |
| | | | | A | | |
| AD-1735309 | CCUGCAGCUGUGCUGGGCAUA | 2544 | 1191-1211 | UAUGCCCAGCACAGCUGCAGGU | 2679 | 1189-1211 |
| | | | | G | | |
| AD-1735310 | AGUGGCCAUGCAGGCGGUGUA | 2545 | 929-949 | UACACCGCCUGCAUGGCCACUG | 2680 | 927-949 |
| | | | | A | | |
| AD-1735311 | GACUUGGCCUUGGGCAGCAUA | 2546 | 663-683 | UAUGCUGCCCAAGGCCAAGUCG | 2681 | 661-683 |
| | | | | G | | |
| AD-1735312 | GAGCGAAGUGCGGGUACCCUA | 2547 | 959-979 | UAGGGUACCCGCACUUCGCUCC | 2682 | 957-979 |
| | | | | U | | |
| AD-1735313 | CUGGUGGCCUCUGUGUGCAAA | 2548 | 279-299 | UUUGCACACAGAGGCCACCAGG | 2683 | 277-299 |
| | | | | G | | |
| AD-1735314 | AACCCACCAGCCGAGGUCGAA | 2549 | 1392-1412 | UUCGACCUCGGCUGGUGGGUUG | 2684 | 1390-1412 |
| | | | | U | | |
| AD-1735315 | UGAUGCUGCCAAGGCGCACUA | 2550 | 1826-1846 | UAGUGCGCCUUGGCAGCAUCAU | 2685 | 1824-1846 |
| | | | | C | | |
| AD-1735316 | CCAAUGAGCUGGCCUGCCGAA | 2551 | 391-411 | UUCGGCAGGCCAGCUCAUUGGC | 2686 | 389-411 |
| | | | | A | | |
| AD-1735317 | GUGCUGCAGCGGGUCCUGCAA | 2552 | 189-209 | UUGCAGGACCCGCUGCAGCACA | 2687 | 187-209 |
| | | | | U | | |
| AD-1735318 | GGGCGUGCAGAGCGCCAGUAA | 2553 | 311-331 | UUACUGGCGCUCUGCACGCCCU | 2688 | 309-331 |
| | | | | U | | |
| AD-1735319 | GGUGAGUGGCACCUGCGAAUA | 2554 | 218-238 | UAUUCGCAGGUGCCACUCACCA | 2689 | 216-238 |
| | | | | C | | |
| AD-1735320 | ACGGAGGGAGAGGACACGGAA | 2555 | 1032-1052 | UUCCGUGUCCUCUCCCUCCGUG | 2690 | 1030-1052 |
| | | | | U | | |

TABLE 11
Modified Sense and Antisense Strand Sequences of PLIN1 dsRNA Agents Comprising a GalNAc Derivative Targeting Ligand

| | | SEQ | | SEQ | | |
|---|---|---|---|---|---|---|
| Duplex | | ID | | ID | | SEQ ID |
| Name | Sense Sequence 5′ to 3′ | NO: | Antisense Sequence 5′ to 3′ | NO: | mRNA Target Sequence 5′ to 3 | NO: |
| AD- | csasucucUfuUfAfAfccaaacuu | 3096 | VPusCfsaagUfuUfGfguuaAfaGfagau | 2826 | UUCAUCUCUUUAACCAAACU | 2961 |
| 1735186 | sgsa | | gsasa | | UGU | |
| AD- | csuscuaaCfaAfAfUfaaacagaas | 3097 | VPusGfsuucUfgUfUfuauuUfgUfuaga | 2827 | UUCUCUAACAAAUAAACAGA | 2962 |
| 1735187 | csa | | gsasa | | ACC | |
| AD- | usgscauaGfuCfAfCfucuuuuga | 3098 | VPusAfsucaAfaAfGfagugAfcUfaugc | 2828 | CCUGCAUAGUCACUCUUUUG | 2963 |
| 1735188 | susa | | asgsg | | AUG | |
| AD- | asascuugAfuUfUfUfucaucucu | 3099 | VPusAfsagaGfaUfGfaaaaAfuCfaagu | 2829 | CUAACUUGAUUUUUCAUCUC | 2964 |
| 1735189 | susa | | usasg | | UUU | |
| AD- | gsascacaUfuCfUfUfagcacuga | 3100 | VPusUfsucaGfuGfCfuaagAfaUfgugu | 2830 | UUGACACAUUCUUAGCACUG | 2965 |
| 1735190 | sasa | | csasa | | AAC | |
| AD- | ascsgccuUfaUfUfUfgauuuaac | 3101 | VPusAfsguuAfaAfUfcaaaUfaAfggcg | 2831 | AUACGCCUUAUUUGAUUUAA | 2966 |
| 1735191 | susa | | usasu | | CUA | |
| AD- | usasgucuUfcGfAfAfauguuaau | 3102 | VPusUfsauuAfaCfAfuuucGfaAfgacu | 2832 | CCUAGUCUUCGAAAUGUUAA | 2967 |
| 1735192 | sasa | | asgsg | | UAU | |
| AD- | gsasuggaCfuUfUfUfaaguuguu | 3103 | VPusAfsaacAfaCfUfuaaaAfgUfccau | 2833 | AUGAUGGACUUUUAAGUUGU | 2968 |
| 1735193 | susa | | csasu | | UUC | |
| AD- | usgscuuuUfuUfCfAfcuuaauaa | 3104 | VPusAfsuuaUfuAfAfgugaAfaAfaagc | 2834 | CCUGCUUUUUUCACUUAAUA | 2969 |
| 1735194 | susa | | asgsg | | AUA | |
| AD- | ascsauaaUfaAfCfUfacugcauas | 3105 | VPusUfsuauGfcAfGfuaguUfaUfuaug | 2835 | CCACAUAAUAACUACUGCAU | 2970 |
| 1735195 | asa | | usgsg | | AAU | |
| AD- | usascuagUfgUfCfAfcuuucuga | 3106 | VPusCfsucaGfaAfAfgugaCfaCfuagu | 2836 | AAUACUAGUGUCACUUUCUG | 2971 |
| 1735196 | sgsa | | asusu | | AGU | |
| AD- | csasaucaGfaUfGfCfaaaagcucs | 3107 | VPusAfsgagCfuUfUfugcaUfcUfgauu | 2837 | AACAAUCAGAUGCAAAAGCU | 2972 |
| 1735197 | usa | | gsusu | | CUU | |
| AD- | usasagagUfaAfUfUfgccuaacu | 3108 | VPusAfsaguUfaGfGfcaauUfaCfucuu | 2838 | UAUAAGAGUAAUUGCCUAAC | 2973 |
| 1735198 | susa | | asusa | | UUG | |
| AD- | gsgscuugUfuUfAfUfgaacauua | 3109 | VPusUfsuaaUfgUfUfcauaAfaCfaagc | 2839 | AUGGCUUGUUUAUGAACAUU | 2974 |
| 1735199 | sasa | | csasu | | AAA | |
| AD- | ususuuugCfuAfCfUfgcaaacga | 3110 | VPusAfsucgUfuUfGfcaguAfgCfaaaa | 2840 | CUUUUUUGCUACUGCAAACG | 2975 |
| 1735200 | susa | | asasg | | AUG | |
| AD- | asascgauGfcUfAfUfaauaaaug | 3111 | VPusAfscauUfuAfUfuauaGfcAfucgu | 2841 | CAAACGAUGCUAUAAUAAAU | 2976 |
| 1735201 | susa | | ususg | | GUC | |
| AD- | usgsguugUfuAfCfUfauaagagu | 3112 | VPusUfsacuCfuUfAfuaguAfaCfaacc | 2842 | CUUGGUUGUUACUAUAAGAG | 2977 |
| 1735202 | sasa | | asasg | | UAA | |
| AD- | csusgaugAfaCfAfUfccucugau | 3113 | VPusCfsaucAfgAfGfgaugUfuCfauca | 2843 | CUCUGAUGAACAUCCUCUGA | 2978 |
| 1735203 | sgsa | | gsasg | | UGA | |
| AD- | csusgcggAfuAfAfAfuauuugcc | 3114 | VPusUfsggcAfaAfUfauuuAfuCfcgca | 2844 | CUCUGCGGAUAAAUAUUUGC | 2979 |
| 1735204 | sasa | | gsasg | | CAC | |
| AD- | cscsaaaaUfuCfAfGfauucugcc | 3115 | VPusAfsggcAfgAfAfucugAfaUfuuu | 2845 | UUCCAAAAUUCAGAUUCUGC | 2980 |
| 1735205 | susa | | ggsasa | | CUC | |
| AD- | asasggauGfuGfUfGfucuuucuc | 3116 | VPusGfsgagAfaAfGfacacAfcAfuccu | 2846 | AAAAGGAUGUGUGUCUUUCU | 2981 |
| 1735206 | scsa | | ususu | | CCC | |
| AD- | usgscgaaUfgCfUfUfccagaaga | 3117 | VPusGfsucuUfcUfGfgaagCfaUfucgc | 2847 | CCUGCGAAUGCUUCCAGAAG | 2982 |
| 1735207 | scsa | | asgsg | | ACC | |
| AD- | csgsaaugAfgUfAfAfcuccuguc | 3118 | VPusUfsgacAfgGfAfguuaCfuCfauuc | 2848 | CACGAAUGAGUAACUCCUGU | 2983 |
| 1735208 | sasa | | gsusg | | CAC | |
| AD- | usgsggugUfuUfAfUfuuaaaaua | 3119 | VPusGfsuauUfuUfAfaauaAfaCfaccc | 2849 | CUUGGGUGUUUAUUUAAAAU | 2984 |
| 1735209 | scsa | | asasg | | ACU | |
| AD- | csasaaagAfuAfUfUfugaccguu | 3120 | VPusAfsaacGfgUfCfaaauAfuCfuuuu | 2850 | GCCAAAAGAUAUUUGACCGU | 2985 |
| 1735210 | susa | | gsgsc | | UUC | |
| AD- | asusacagCfuUfGfGfagagauuu | 3121 | VPusAfsaaaUfcUfCfuccaAfgCfugua | 2851 | UAAUACAGCUUGGAGAGAUU | 2986 |
| 1735211 | susa | | ususa | | UUU | |
| AD- | asasaugcAfuUfCfAfuacaauua | 3122 | VPusGfsuaaUfuGfUfaugaAfuGfcauu | 2852 | GAAAAUGCAUUCAUACAAUU | 2987 |
| 1735212 | scsa | | ususc | | ACA | |
| AD- | uscsccacUfcGfCfUfcuuuuuga | 3123 | VPusAfsucaAfaAfAfgagcGfaGfuggg | 2853 | AAUCCCACUCGCUCUUUUUG | 2988 |
| 1735213 | susa | | asusu | | AUG | |
| AD- | gscsuuguGfuUfUfAfcacaagcu | 3124 | VPusCfsagcUfuGfUfguaaAfcAfcaag | 2854 | CCGCUUGUGUUUACACAAGC | 2989 |
| 1735214 | sgsa | | csgsg | | UGU | |
| AD- | cscsugccUfuAfCfAfuggcuugu | 3125 | VPusAfsacaAfgCfCfauguAfaGfgcag | 2855 | CACCUGCCUUACAUGGCUUG | 2990 |
| 1735215 | susa | | gsusg | | UUU | |
| AD- | gsasgauuUfuUfGfUfaucacauu | 3126 | VPusUfsaauGfuGfAfuacaAfaAfaucu | 2856 | GAGAGAUUUUUGUAUCACAU | 2991 |
| 1735216 | sasa | | csusc | | UAU | |
| AD- | asgscacuUfcAfGfAfcaaggucc | 3127 | VPusAfsggaCfcUfUfgucuGfaAfgugc | 2857 | CGAGCACUUCAGACAAGGUC | 2992 |
| 1735217 | susa | | uscsg | | CUG | |
| AD- | uscsacucUfcAfGfAfgccaguuu | 3128 | VPusAfsaaaCfuGfGfcucuGfaGfagug | 2858 | CUUCACUCUCAGAGCCAGUU | 2993 |
| 1735218 | susa | | asasg | | UUU | |
| AD- | ascsugaaCfuCfCfUfcugugauc | 3129 | VPusAfsgauCfaCfAfgaggAfgUfucag | 2859 | GCACUGAACUCCUCUGUGAU | 2994 |
| 1735219 | susa | | usgsc | | CUA | |
| AD- | usasguucCfcUfAfAfugauggac | 3130 | VPusAfsgucCfaUfCfauuaGfgGfaacu | 2860 | ACUAGUUCCCUAAUGAUGGA | 2995 |
| 1735220 | susa | | asgsu | | CUU | |
| AD- | gsgscguuAfcUfGfAfcaacgugg | 3131 | VPusAfsccaCfgUfUfgucaGfuAfacgc | 2861 | AGGGCGUUACUGACAACGUG | 2996 |
| 1735221 | susa | | cscsu | | GUG | |
| AD- | usgsaucuAfgGfAfUfgaucugu | 3132 | VPusGfsaacAfgAfUfcaucCfuAfgauc | 2862 | UGUGAUCUAGGAUGAUCUGU | 2997 |
| 1735222 | uscsa | | ascsa | | UCC | |
| AD- | gsusgaugAfuAfUfAfuagacuu | 3133 | VPusUfsaaaGfuCfUfauauAfuCfauca | 2863 | UGGUGAUGAUAUAUAGACUU | 2998 |
| 1735223 | usasa | | cscsa | | UAU | |
| AD- | gsgscuacUfuUfGfAfagggaaca | 3134 | VPusUfsuguUfcCfCfuucaAfaGfuagc | 2864 | CAGGCUACUUUGAAGGGAAC | 2999 |
| 1735224 | sasa | | csusg | | AAU | |
| AD- | csusgcucUfgAfUfUfcuauggcu | 3135 | VPusAfsagcCfaUfAfgaauCfaGfagca | 2865 | GCCUGCUCUGAUUCUAUGGC | 3000 |
| 1735225 | susa | | gsgsc | | UUG | |
| AD- | asgsauugCfuUfCfUfgagcugaa | 3136 | VPusCfsuucAfgCfUfcagaAfgCfaauc | 2866 | AAAGAUUGCUUCUGAGCUGA | 3001 |
| 1735226 | sgsa | | ususu | | AGG | |
| AD- | gscscaucUfcUfUfGfuguaugca | 3137 | VPusCfsugcAfuAfCfacaaGfaGfaugg | 2867 | GUGCCAUCUCUUGUGUAUGC | 3002 |
| 1735227 | sgsa | | csasc | | AGG | |
| AD- | asgsccacAfuUfUfCfcauuugca | 3138 | VPusAfsugcAfaAfUfggaaAfuGfuggc | 2868 | ACAGCCACAUUUCCAUUUGC | 3003 |
| 1735228 | susa | | usgsu | | AUC | |
| AD- | uscscagaCfaGfAfAfgguuuuga | 3139 | VPusGfsucaAfaAfCfcuucUfgUfcugg | 2869 | GGUCCAGACAGAAGGUUUUG | 3004 |
| 1735229 | scsa | | ascsc | | ACA | |
| AD- | csuscucgAfuAfCfAfccgugcag | 3140 | VPusUfscugCfaCfGfguguAfuCfgaga | 2870 | CUCUCUCGAUACACCGUGCA | 3005 |
| 1735230 | sasa | | gsasg | | GAC | |
| AD- | gsgscgcaCfuUfUfUfuauuuuua | 3141 | VPusAfsuaaAfaAfUfaaaaAfgUfgcgc | 2871 | AAGGCGCACUUUUUAUUUUU | 3006 |
| 1735231 | susa | | csusu | | AUU | |
| AD- | gsasgcguUfgCfCfAfcuuucaaa | 3142 | VPusCfsuuuGfaAfAfguggCfaAfcgcu | 2872 | GCGAGCGUUGCCACUUUCAA | 3007 |
| 1735232 | sgsa | | csgsc | | AGU | |
| AD- | ususgcauCfaUfUfAfcugccuuc | 3143 | VPusUfsgaaGfgCfAfguaaUfgAfugca | 2873 | AUUUGCAUCAUUACUGCCUU | 3008 |
| 1735233 | sasa | | asasu | | CAC | |
| AD- | gscsgcaaGfaAfGfAfgcugaguc | 3144 | VPusCfsgacUfcAfGfcucuUfcUfugcg | 2874 | CUGCGCAAGAAGAGCUGAGU | 3009 |
| 1735234 | sgsa | | csasg | | CGC | |
| AD- | usgscggaAfuUfUfGfcugccaac | 3145 | VPusUfsguuGfgCfAfgcaaAfuUfccgc | 2875 | ACUGCGGAAUUUGCUGCCAA | 3010 |
| 1735235 | sasa | | asgsu | | CAC | |
| AD- | gsusgugcAfaUfGfCfcuaugaga | 3146 | VPusUfsucuCfaUfAfggcaUfuGfcaca | 2876 | CUGUGUGCAAUGCCUAUGAG | 3011 |
| 1735236 | sasa | | csasg | | AAG | |
| AD- | gsasauucAfaAfUfAfuugcaaaa | 3147 | VPusCfsuuuUfgCfAfauauUfuGfaauu | 2877 | CAGAAUUCAAAUAUUGCAAA | 3012 |
| 1735237 | sgsa | | csusg | | AGG | |
| AD- | cscsugugUfgCfUfUfuguagagc | 3148 | VPusGfsgcuCfuAfCfaaagCfaCfacag | 2878 | GGCCUGUGUGCUUUGUAGAG | 3013 |
| 1735238 | scsa | | gscsc | | CCA | |
| AD- | uscscagaCfaAfGfGfaagaguca | 3149 | VPusCfsugaCfuCfUfuccuUfgUfcugg | 2879 | CCUCCAGACAAGGAAGAGUC | 3014 |
| 1735239 | sgsa | | asgsg | | AGC | |
| AD- | usgsccagAfaAfCfAfgcaucagc | 3150 | VPusCfsgcuGfaUfGfcuguUfuCfuggc | 2880 | AGUGCCAGAAACAGCAUCAG | 3015 |
| 1735240 | sgsa | | ascsu | | CGU | |
| AD- | gscsauaaUfaUfGfGfauacgccu | 3151 | VPusAfsaggCfgUfAfuccaUfaUfuaug | 2881 | CUGCAUAAUAUGGAUACGCC | 3016 |
| 1735241 | susa | | csasg | | UUA | |
| AD- | asgsccucAfuCfCfUfgaugaugc | 3152 | VPusAfsgcaUfcAfUfcaggAfuGfaggc | 2882 | UCAGCCUCAUCCUGAUGAUG | 3017 |
| 1735242 | susa | | usgsa | | CUG | |
| AD- | asasgccuCfuUfGfAfgcaggguu | 3153 | VPusCfsaacCfcUfGfcucaAfgAfggcu | 2883 | CCAAGCCUCUUGAGCAGGGU | 3018 |
| 1735243 | sgsa | | usgsg | | UGG | |
| AD- | usasaaggGfaAfGfAfaguugaag | 3154 | VPusGfscuuCfaAfCfuucuUfcCfcuuu | 2884 | AUUAAAGGGAAGAAGUUGAA | 3019 |
| 1735244 | scsa | | asasu | | GCU | |
| AD- | gsgscagcAfuUfGfAfgaaggugg | 3155 | VPusAfsccaCfcUfUfcucaAfuGfcugc | 2885 | UGGGCAGCAUUGAGAAGGUG | 3020 |
| 1735245 | susa | | cscsa | | GUG | |
| AD- | gsuscagcGfaCfAfGfcuucuucc | 3156 | VPusCfsggaAfgAfAfgcugUfcGfcuga | 2886 | GGGUCAGCGACAGCUUCUUC | 3021 |
| 1735246 | sgsa | | cscsc | | CGG | |
| AD- | csusgcugUfcUfCfCfucaaccaas | 3157 | VPusCfsuugGfuUfGfaggaGfaCfagca | 2887 | CCCUGCUGUCUCCUCAACCAA | 3022 |
| 1735247 | gsa | | gsgsg | | GG | |
| AD- | asgsgaggAfaGfAfAfuuggagac | 3158 | VPusAfsgucUfcCfAfauucUfuCfcucc | 2888 | GGAGGAGGAAGAAUUGGAGA | 3023 |
| 1735248 | susa | | uscsc | | CUG | |
| AD- | cscsugucAfcCfAfCfucugaagg | 3159 | VPusAfsccuUfcAfGfagugGfuGfacag | 2889 | CUCCUGUCACCACUCUGAAG | 3024 |
| 1735249 | susa | | gsasg | | GUC | |
| AD- | asgsacuuUfaUfGfUfauagccac | 3160 | VPusUfsgugGfcUfAfuacaUfaAfaguc | 2890 | AUAGACUUUAUGUAUAGCCA | 3025 |
| 1735250 | sasa | | usasu | | CAG | |
| AD- | gsusuacgCfaUfGfCfcugcuuuu | 3161 | VPusAfsaaaAfgCfAfggcaUfgCfguaa | 2891 | GUGUUACGCAUGCCUGCUUU | 3026 |
| 1735251 | susa | | csasc | | UUU | |
| AD- | cscsaguuCfaCfAfGfcugccaau | 3162 | VPusCfsauuGfgCfAfgcugUfgAfacug | 2892 | ACCCAGUUCACAGCUGCCAA | 3027 |
| 1735252 | sgsa | | gsgsu | | UGA | |
| AD- | csasggcaCfgUfGfUfguaugcac | 3163 | VPusAfsgugCfaUfAfcacaCfgUfgccu | 2893 | UGCAGGCACGUGUGUAUGCA | 3028 |
| 1735253 | susa | | gscsa | | CUC | |
| AD- | asusggcaGfuCfAfAfcaaaggcc | 3164 | VPusAfsggcCfuUfUfguugAfcUfgcca | 2894 | GGAUGGCAGUCAACAAAGGC | 3029 |
| 1735254 | susa | | uscsc | | CUC | |
| AD- | csasaacuUfgUfGfGfccaaaagas | 3165 | VPusAfsucuUfuUfGfgccaCfaAfguuu | 2895 | ACCAAACUUGUGGCCAAAAG | 3030 |
| 1735255 | usa | | gsgsu | | AUA | |
| AD- | uscsugacCfaAfCfAfcccucucu | 3166 | VPusGfsagaGfaGfGfguguUfgGfucag | 2896 | GCUCUGACCAACACCCUCUCU | 3031 |
| 1735256 | scsa | | asgsc | | CG | |
| AD- | uscsacauUfaUfAfAfaucccacu | 3167 | VPusGfsaguGfgGfAfuuuaUfaAfugu | 2897 | UAUCACAUUAUAAAUCCCAC | 3032 |
| 1735257 | scsa | | gasusa | | UCG | |
| AD- | gsasggagAfaCfAfAfguucagug | 3168 | VPusUfscacUfgAfAfcuugUfuCfuccu | 2898 | CUGAGGAGAACAAGUUCAGU | 3033 |
| 1735258 | sasa | | csasg | | GAG | |
| AD- | gsasgcagGfaGfAfAfugugcugc | 3169 | VPusUfsgcaGfcAfCfauucUfcCfugcu | 2899 | CUGAGCAGGAGAAUGUGCUG | 3034 |
| 1735259 | sasa | | csasg | | CAG | |
| AD- | gsasagacCfuAfCfAfccagcacus | 3170 | VPusUfsaguGfcUfGfguguAfgGfucu | 2900 | CAGAAGACCUACACCAGCAC | 3035 |
| 1735260 | asa | | ucsusg | | UAA | |
| AD- | asgsgccuCfaCfCfUfugcuggau | 3171 | VPusCfsaucCfaGfCfaaggUfgAfggcc | 2901 | AAAGGCCUCACCUUGCUGGA | 3036 |
| 1735261 | sgsa | | ususu | | UGG | |
| AD- | asgsccagCfuGfCfUfugcucaca | 3172 | VPusCfsuguGfaGfCfaagcAfgCfuggc | 2902 | AGAGCCAGCUGCUUGCUCAC | 3037 |
| 1735262 | sgsa | | uscsu | | AGC | |
| AD- | gsgsgcucUfgUfGfAfgacugagg | 3173 | VPusAfsccuCfaGfUfcucaCfaGfagccc | 2903 | CUGGGCUCUGUGAGACUGAG | 3038 |
| 1735263 | susa | | sasg | | GUG | |
| AD- | csgsagagCfgAfAfUfuccgggac | 3174 | VPusUfsgucCfcGfGfaauuCfgCfucuc | 2904 | CCCGAGAGCGAAUUCCGGGA | 3039 |
| 1735264 | sasa | | gsgsg | | CAU | |
| AD- | gsusggcaCfaUfAfCfccugcaga | 3175 | VPusUfsucuGfcAfGfgguaUfgUfgcca | 2905 | GUGUGGCACAUACCCUGCAG | 3040 |
| 1735265 | sasa | | csasc | | AAG | |
| AD- | gsusggacAfcAfGfUfggugcauu | 3176 | VPusUfsaauGfcAfCfcacuGfuGfucca | 2906 | UGGUGGACACAGUGGUGCAU | 3041 |
| 1735266 | sasa | | cscsa | | UAC | |
| AD- | asasgcccAfgAfAfGfucucccaa | 3177 | VPusCfsuugGfgAfGfacuuCfuGfggcu | 2907 | GCAAGCCCAGAAGUCUCCCA | 3042 |
| 1735267 | sgsa | | usgsc | | AGG | |
| AD- | gsgsccauGfuCfCfCfuaucagau | 3178 | VPusCfsaucUfgAfUfagggAfcAfuggc | 2908 | AGGGCCAUGUCCCUAUCAGA | 3043 |
| 1735268 | sgsa | | cscsu | | UGC | |
| AD- | gscscggcGfgGfUfUfucucuaac | 3179 | VPusUfsguuAfgAfGfaaacCfcGfccgg | 2909 | GGGCCGGCGGGUUUCUCUAA | 3044 |
| 1735269 | sasa | | cscsc | | CAA | |
| AD- | csusgaagGfaCfAfCfcaucuccas | 3180 | VPusGfsuggAfgAfUfggugUfcCfuuca | 2910 | AGCUGAAGGACACCAUCUCC | 3045 |
| 1735270 | csa | | gscsu | | ACC | |
| AD- | gsasccacCfaUfCfUfcggcugug | 3181 | VPusUfscacAfgCfCfgagaUfgGfuggu | 2911 | CAGACCACCAUCUCGGCUGU | 3046 |
| 1735271 | sasa | | csusg | | GAC | |
| AD- | cscsaguuUfuUfAfAfgggacacc | 3182 | VPusUfsgguGfuCfCfcuuaAfaAfacug | 2912 | AGCCAGUUUUUAAGGGACAC | 3047 |
| 1735272 | sasa | | gscsu | | CAG | |
| AD- | gsgsccagAfgAfCfAfcugcggaa | 3183 | VPusAfsuucCfgCfAfguguCfuCfuggc | 2913 | GUGGCCAGAGACACUGCGGA | 3048 |
| 1735273 | susa | | csasc | | AUU | |
| AD- | csgsuuccCfaUfCfGfcgagcacu | 3184 | VPusAfsaguGfcUfCfgcgaUfgGfgaac | 2914 | AGCGUUCCCAUCGCGAGCAC | 3049 |
| 1735274 | susa | | gscsu | | UUC | |
| AD- | cscsgggaCfaUfCfGfacaacccas | 3185 | VPusGfsuggGfuUfGfucgaUfgUfcccg | 2915 | UUCCGGGACAUCGACAACCC | 3050 |
| 1735275 | csa | | gsasa | | ACC | |
| AD- | uscsugauGfaUfCfUfaggcuccc | 3186 | VPusUfsgggAfgCfCfuagaUfcAfucag | 2916 | CCUCUGAUGAUCUAGGCUCC | 3051 |
| 1735276 | sasa | | asgsg | | CAG | |
| AD- | csusgcagAfaGfAfCfccuccaga | 3187 | VPusGfsucuGfgAfGfggucUfuCfugca | 2917 | CCCUGCAGAAGACCCUCCAGA | 3052 |
| 1735277 | scsa | | gsgsg | | CC | |
| AD- | csgsccagUfaGfCfUfuggcugcc | 3188 | VPusAfsggcAfgCfCfaagcUfaCfuggc | 2918 | AGCGCCAGUAGCUUGGCUGC | 3053 |
| 1735278 | susa | | gscsu | | CUG | |
| AD- | csgscuucAfcAfGfGfcugagucc | 3189 | VPusUfsggaCfuCfAfgccuGfuGfaagc | 2919 | GCCGCUUCACAGGCUGAGUC | 3054 |
| 1735279 | sasa | | gsgsc | | CAG | |
| AD- | ususuuuuUfaGfCfAfuccuuuu | 3190 | VPusCfscaaAfaGfGfaugcUfaAfaaaaa | 2920 | UUUUUUUUUAGCAUCCUUUU | 3055 |
| 1735280 | gsgsa | | sasa | | GGG | |
| AD- | gsasagccAfaAfGfCfgcaggguc | 3191 | VPusUfsgacCfcUfGfcgcuUfuGfgcuu | 2921 | GAGAAGCCAAAGCGCAGGGU | 3056 |
| 1735281 | sasa | | csusc | | CAG | |
| AD- | gsasggagGfaUfCfAfugaggacc | 3192 | VPusUfsgguCfcUfCfaugaUfcCfuccu | 2922 | AGGAGGAGGAUCAUGAGGAC | 3057 |
| 1735282 | sasa | | cscsu | | CAG | |
| AD- | gscsacgcAfuUfAfCfagccagcu | 3193 | VPusCfsagcUfgGfCfuguaAfuGfcgug | 2923 | CCGCACGCAUUACAGCCAGCU | 3058 |
| 1735283 | sgsa | | csgsg | | GC | |
| AD- | csusucauUfgAfAfAfaccaccac | 3194 | VPusCfsgugGfuGfGfuuuuCfaAfugaa | 2924 | CCCUUCAUUGAAAACCACCAC | 3059 |
| 1735284 | sgsa | | gsgsg | | GG | |
| AD- | ususuuugAfuGfGfCfcacauaau | 3195 | VPusUfsauuAfuGfUfggccAfuCfaaaa | 2925 | UCUUUUUGAUGGCCACAUAA | 3060 |
| 1735285 | sasa | | asgsa | | UAA | |
| AD- | cscsaccuGfgAfGfGfaaaagauc | 3196 | VPusGfsgauCfuUfUfuccuCfcAfggug | 2926 | GACCACCUGGAGGAAAAGAU | 3061 |
| 1735286 | scsa | | gsusc | | CCC | |
| AD- | csasaggcCfaAfGfCfcaagccucs | 3197 | VPusAfsgagGfcUfUfggcuUfgGfccuu | 2927 | CCCAAGGCCAAGCCAAGCCUC | 3062 |
| 1735287 | usa | | gsgsg | | UU | |
| AD- | gsasggacCfaGfAfCfagacacgg | 3198 | VPusUfsccgUfgUfCfugucUfgGfuccu | 2928 | AUGAGGACCAGACAGACACG | 3063 |
| 1735288 | sasa | | csasu | | GAG | |
| AD- | gsasugccCfuGfAfAfgggcguua | 3199 | VPusGfsuaaCfgCfCfcuucAfgGfgcau | 2929 | CAGAUGCCCUGAAGGGCGUU | 3064 |
| 1735289 | scsa | | csusg | | ACU | |
| AD- | cscsaacaCfuCfGfAfgcuggccg | 3200 | VPusUfscggCfcAfGfcucgAfgUfguug | 2930 | UGCCAACACUCGAGCUGGCC | 3065 |
| 1735290 | sasa | | gscsa | | GAC | |
| AD- | csasgggcUfaUfGfCfaccugcag | 3201 | VPusCfscugCfaGfGfugcaUfaGfcccu | 2931 | UGCAGGGCUAUGCACCUGCA | 3066 |
| 1735291 | sgsa | | gscsa | | GGC | |
| AD- | asasggugGfuGfGfAfguaccucc | 3202 | VPusAfsggaGfgUfAfcuccAfcCfaccu | 2932 | AGAAGGUGGUGGAGUACCUC | 3067 |
| 1735292 | susa | | uscsu | | CUC | |
| AD- | ususgaagCfuUfGfAfggagcgag | 3203 | VPusCfscucGfcUfCfcucaAfgCfuuca | 2933 | AGUUGAAGCUUGAGGAGCGA | 3068 |
| 1735293 | sgsa | | ascsu | | GGA | |
| AD- | csgsuggcCfaUfGfUfggauccca | 3204 | VPusCfsuggGfaUfCfcacaUfgGfccac | 2934 | ACCGUGGCCAUGUGGAUCCC | 3069 |
| 1735294 | sgsa | | gsgsu | | AGG | |
| AD- | csasgcacUfaAfGfGfaagcccacs | 3205 | VPusGfsgugGfgCfUfuccuUfaGfugcu | 2935 | ACCAGCACUAAGGAAGCCCA | 3070 |
| 1735295 | csa | | gsgsu | | CCC | |
| AD- | gsuscgcuGfaUfGfGfagcccgag | 3206 | VPusUfscucGfgGfCfuccaUfcAfgcga | 2936 | CUGUCGCUGAUGGAGCCCGA | 3071 |
| 1735296 | sasa | | csasg | | GAG | |
| AD- | ususcuucCfgGfCfCfcagcguca | 3207 | VPusAfsugaCfgCfUfgggcCfgGfaaga | 2937 | GCUUCUUCCGGCCCAGCGUCA | 3072 |
| 1735297 | susa | | asgsc | | UG | |
| AD- | csusgcucCfuGfGfAfcaccagca | 3208 | VPusUfsugcUfgGfUfguccAfgGfagca | 2938 | CCCUGCUCCUGGACACCAGCA | 3073 |
| 1735298 | sasa | | gsgsg | | AG | |
| AD- | ususucugAfgUfAfCfccgccgcu | 3209 | VPusAfsagcGfgCfGfgguaCfuCfagaa | 2939 | ACUUUCUGAGUACCCGCCGC | 3074 |
| 1735299 | susa | | asgsu | | UUC | |
| AD- | csgscaggCfuGfUfCfcacccagu | 3210 | VPusAfsacuGfgGfUfggacAfgCfcugc | 2940 | UCCGCAGGCUGUCCACCCAGU | 3075 |
| 1735300 | susa | | gsgsa | | UC | |
| AD- | gsgscuguGfaCfAfUfgggcaccu | 3211 | VPusCfsaggUfgCfCfcaugUfcAfcagc | 2941 | UCGGCUGUGACAUGGGCACC | 3076 |
| 1735301 | sgsa | | csgsa | | UGC | |
| AD- | uscsagugAfgGfUfAfgcagcccu | 3212 | VPusCfsaggGfcUfGfcuacCfuCfacug | 2942 | GUUCAGUGAGGUAGCAGCCC | 3077 |
| 1735302 | sgsa | | asasc | | UGC | |
| AD- | asgscgucAfuGfGfAfgcccaucc | 3213 | VPusAfsggaUfgGfGfcuccAfuGfacgc | 2943 | CCAGCGUCAUGGAGCCCAUCC | 3078 |
| 1735303 | susa | | usgsg | | UG | |
| AD- | csusggauGfgAfGfAfccucccug | 3214 | VPusUfscagGfgAfGfgucuCfcAfucca | 2944 | UGCUGGAUGGAGACCUCCCU | 3079 |
| 1735304 | sasa | | gscsa | | GAG | |
| AD- | uscsagccGfgAfGfUfgaguguug | 3215 | VPusCfscaaCfaCfUfcacuCfcGfgcuga | 2945 | GGUCAGCCGGAGUGAGUGUU | 3080 |
| 1735305 | sgsa | | scsc | | GGG | |
| AD- | csusggccGfaCfUfGfgcuucugg | 3216 | VPusUfsccaGfaAfGfccagUfcGfgcca | 2946 | AGCUGGCCGACUGGCUUCUG | 3081 |
| 1735306 | sasa | | gscsu | | GAG | |
| AD- | gsgsugcuGfcAfCfCfucacacca | 3217 | VPusCfsuggUfgUfGfagguGfcAfgcac | 2947 | AGGGUGCUGCACCUCACACC | 3082 |
| 1735307 | sgsa | | cscsu | | AGC | |
| AD- | gsgscugcCfuGfGfAfgcauggag | 3218 | VPusGfscucCfaUfGfcuccAfgGfcagc | 2948 | UUGGCUGCCUGGAGCAUGGA | 3083 |
| 1735308 | scsa | | csasa | | GCC | |
| AD- | cscsugcaGfcUfGfUfgcugggca | 3219 | VPusAfsugcCfcAfGfcacaGfcUfgcag | 2949 | CACCUGCAGCUGUGCUGGGC | 3084 |
| 1735309 | susa | | gsusg | | AUG | |
| AD- | asgsuggcCfaUfGfCfaggcggug | 3220 | VPusAfscacCfgCfCfugcaUfgGfccac | 2950 | UCAGUGGCCAUGCAGGCGGU | 3085 |
| 1735310 | susa | | usgsa | | GUC | |
| AD- | gsascuugGfcCfUfUfgggcagca | 3221 | VPusAfsugcUfgCfCfcaagGfcCfaagu | 2951 | CCGACUUGGCCUUGGGCAGC | 3086 |
| 1735311 | susa | | csgsg | | AUU | |
| AD- | gsasgcgaAfgUfGfCfggguaccc | 3222 | VPusAfsgggUfaCfCfcgcaCfuUfcgcu | 2952 | AGGAGCGAAGUGCGGGUACC | 3087 |
| 1735312 | susa | | cscsu | | CUG | |
| AD- | csusggugGfcCfUfCfugugugca | 3223 | VPusUfsugcAfcAfCfagagGfcCfacca | 2953 | CCCUGGUGGCCUCUGUGUGC | 3088 |
| 1735313 | sasa | | gsgsg | | AAU | |
| AD- | asascccaCfcAfGfCfcgaggucgs | 3224 | VPusUfscgaCfcUfCfggcuGfgUfgggu | 2954 | ACAACCCACCAGCCGAGGUCG | 3089 |
| 1735314 | asa | | usgsu | | AG | |
| AD- | usgsaugcUfgCfCfAfaggcgcac | 3225 | VPusAfsgugCfgCfCfuuggCfaGfcauc | 2955 | GAUGAUGCUGCCAAGGCGCA | 3090 |
| 1735315 | susa | | asusc | | CUU | |
| AD- | cscsaaugAfgCfUfGfgccugccg | 3226 | VPusUfscggCfaGfGfccagCfuCfauug | 2956 | UGCCAAUGAGCUGGCCUGCC | 3091 |
| 1735316 | sasa | | gscsa | | GAG | |
| AD- | gsusgcugCfaGfCfGfgguccugc | 3227 | VPusUfsgcaGfgAfCfccgcUfgCfagca | 2957 | AUGUGCUGCAGCGGGUCCUG | 3092 |
| 1735317 | sasa | | csasu | | CAG | |
| AD- | gsgsgcguGfcAfGfAfgcgccagu | 3228 | VPusUfsacuGfgCfGfcucuGfcAfcgcc | 2958 | AAGGGCGUGCAGAGCGCCAG | 3093 |
| 1735318 | sasa | | csusu | | UAG | |
| AD- | gsgsugagUfgGfCfAfccugcgaa | 3229 | VPusAfsuucGfcAfGfgugcCfaCfucac | 2959 | GUGGUGAGUGGCACCUGCGA | 3094 |
| 1735319 | susa | | csasc | | AUG | |
| AD- | ascsggagGfgAfGfAfggacacgg | 3230 | VPusUfsccgUfgUfCfcucuCfcCfuccg | 2960 | ACACGGAGGGAGAGGACACG | 3095 |
| 1735320 | sasa | | usgsu | | GAG | |

TABLE 12
Unmodified Sense and Antisense Strand Sequences of PDE3B dsRNA Agents Comprising an Unsaturated
C22 Hydrocarbon Chain Conjugated to Position 6 on the Sense Strand, Counting from the 5′-end of
the Sense Strand

| | | SEQ | Range in | | SEQ | Range in |
|---|---|---|---|---|---|---|
| Duplex | Sense Sequence | ID | NM_ | Antisense Sequence | ID | NM_ |
| Name | 5′ to 3′ | NO: | 000922.4 | 5′ to 3′ | NO: | 000922.4 |
| AD-1735321 | UUCCUUCUUCAUCUUGAUCAA | 3231 | 2880-2900 | UUGAUCAAGAUGAAGAAGGAAG | 3500 | 2878-2900 |
| | | | | U | | |
| AD-1735322 | CACUCUAAAACUGAUGUUCA | 3232 | 4621-4641 | UUGAACAUCAGUUUUAGAGUGA | 3501 | 4619-4641 |
| | A | | | A | | |
| AD-1735323 | UCUUCUGUAUCACUGACUCAA | 3233 | 1752-1772 | UUGAGUCAGUGAUACAGAAGAU | 3502 | 1750-1772 |
| | | | | G | | |
| AD-1735324 | CAGAUCUUCUGACUGAUCCAA | 3234 | 1363-1383 | UUGGAUCAGUCAGAAGAUCUGA | 3503 | 1361-1383 |
| | | | | C | | |
| AD-1735325 | UGACACAUUUUUCUCCAUAA | 3235 | 5958-5978 | UUUAUGGAGAAAAAUGUGUCAG | 3504 | 5956-5978 |
| | A | | | U | | |
| AD-1735326 | GGAAACUCAUUUUUACAUAA | 3236 | 5617-5637 | UUUAUGUAAAAAUGAGUUUCCA | 3505 | 5615-5637 |
| | A | | | G | | |
| AD-1735327 | AUCAGCAACUUAGAAAUUCU | 3237 | 1966-1986 | UAGAAUUUCUAAGUUGCUGAUA | 3506 | 1964-1986 |
| | A | | | A | | |
| AD-1735328 | CUUAUAUUUCUCUAGAGUAC | 3238 | 5351-5371 | UGUACUCUAGAGAAAUAUAAGG | 3507 | 5349-5371 |
| | A | | | A | | |
| AD-1735329 | AACUCCAAGAAUCUUUUAUC | 3239 | 3229-3249 | UGAUAAAAGAUUCUUGGAGUUU | 3508 | 3227-3249 |
| | A | | | U | | |
| AD-1735330 | CUUCAAACAUUCCUGCAUUAA | 3240 | 2680-2700 | UUAAUGCAGGAAUGUUUGAAGA | 3509 | 2678-2700 |
| | | | | C | | |
| AD-1735331 | UCGUUCUUCUCCUCAACUAGA | 3241 | 3206-3226 | UCUAGUUGAGGAGAAGAACGAU | 3510 | 3204-3226 |
| | | | | C | | |
| AD-1735332 | AAAGAUUAAUCCUCUCACACA | 3242 | 1460-1480 | UGUGUGAGAGGAUUAAUCUUUG | 3511 | 1458-1480 |
| | | | | G | | |
| AD-1735333 | ACUAACCUCAUCAAAGAUACA | 3243 | 4501-4521 | UGUAUCUUUGAUGAGGUUAGUU | 3512 | 4499-4521 |
| | | | | U | | |
| AD-1735334 | CACCACUUAUUACUAAUAAA | 3244 | 5283-5303 | UUUUAUUAGUAAUAAGUGGUGG | 3513 | 5281-5303 |
| | A | | | G | | |
| AD-1735335 | UGGUACUAGAAAUAUUCUUU | 3245 | 5915-5935 | UAAAGAAUAUUUCUAGUACCAG | 3514 | 5913-5935 |
| | A | | | C | | |
| AD-1735336 | GCUGUCAUUUGUUCACUCUA | 3246 | 4608-4628 | UUAGAGUGAACAAAUGACAGCU | 3515 | 4606-4628 |
| | A | | | A | | |
| AD-1735337 | ACUCAACAAUUUAUGAACUA | 3247 | 2421-2441 | UUAGUUCAUAAAUUGUUGAGUG | 3516 | 2419-2441 |
| | A | | | G | | |
| AD-1735338 | AGUAUGACUCAUUAAUAGAA | 3248 | 2269-2289 | UUUCUAUUAAUGAGUCAUACUC | 3517 | 2267-2289 |
| | A | | | U | | |
| AD-1735339 | CUGCUGAUUUUCUUAAUAAG | 3249 | 1891-1911 | UCUUAUUAAGAAAAUCAGCAGU | 3518 | 1889-1911 |
| | A | | | A | | |
| AD-1735340 | UAUCUUUAGAUCCACAUCUU | 3250 | 4587-4607 | UAAGAUGUGGAUCUAAAGAUAA | 3519 | 4585-4607 |
| | A | | | C | | |
| AD-1735341 | CGAGACAUUCCUUAUCACAAA | 3251 | 2466-2486 | UUUGUGAUAAGGAAUGUCUCGA | 3520 | 2464-2486 |
| | | | | U | | |
| AD-1735342 | UACUGAAUCAGAUUAAUUUC | 3252 | 5850-5870 | UGAAAUUAAUCUGAUUCAGUAA | 3521 | 5848-5870 |
| | A | | | C | | |
| AD-1735343 | CUUACUUAAAUCCUUCACUGA | 3253 | 3855-3875 | UCAGUGAAGGAUUUAAGUAAGG | 3522 | 3853-3875 |
| | | | | G | | |
| AD-1735344 | UCGAAAGAAUCAUUCAAACU | 3254 | 2100-2120 | UAGUUUGAAUGAUUCUUUCGAG | 3523 | 2098-2120 |
| | A | | | A | | |
| AD-1735345 | AAAUGACCUAUCACUACUUA | 3255 | 4682-4702 | UUAAGUAGUGAUAGGUCAUUUC | 3524 | 4680-4702 |
| | A | | | U | | |
| AD-1735346 | CAAGUCAUUUCCUCUCUACGA | 3256 | 1395-1415 | UCGUAGAGAGGAAAUGACUUGU | 3525 | 1393-1415 |
| | | | | G | | |
| AD-1735347 | AUGCAGUUUCUUACUUAUCU | 3257 | 5079-5099 | UAGAUAAGUAAGAAACUGCAUG | 3526 | 5077-5099 |
| | A | | | C | | |
| AD-1735348 | UCAAUGCAAUACACUGUUCA | 3258 | 4124-4144 | UUGAACAGUGUAUUGCAUUGAG | 3527 | 4122-4144 |
| | A | | | C | | |
| AD-1735349 | UCUAUGUACCUACUGACACAA | 3259 | 5945-5965 | UUGUGUCAGUAGGUACAUAGAU | 3528 | 5943-5965 |
| | | | | A | | |
| AD-1735350 | UGAAUUCCAGUCUUAUCUUA | 3260 | 4312-4332 | UUAAGAUAAGACUGGAAUUCAA | 3529 | 4310-4332 |
| | A | | | A | | |
| AD-1735351 | UUGCUGUAAUACCAAAACUA | 3261 | 4485-4505 | UUAGUUUUGGUAUUACAGCAAA | 3530 | 4483-4505 |
| | A | | | U | | |
| AD-1735352 | GGAUCACUUCUUUCAAAUCA | 3262 | 1061-1081 | UUGAUUUGAAAGAAGUGAUCCA | 3531 | 1059-1081 |
| | A | | | A | | |
| AD-1735353 | GAACUAGAAAACUAUUCUUA | 3263 | 3795-3815 | UUAAGAAUAGUUUUCUAGUUCC | 3532 | 3793-3815 |
| | A | | | U | | |
| AD-1735354 | ACAUUUCCAUACUGUCUGUU | 3264 | 5484-5504 | UAACAGACAGUAUGGAAAUGUG | 3533 | 5482-5504 |
| | A | | | A | | |
| AD-1735355 | UGAUCCUUCACAGUGUCAAU | 3265 | 5158-5178 | UAUUGACACUGUGAAGGAUCAA | 3534 | 5156-5178 |
| | A | | | A | | |
| AD-1735356 | GAUUUCUGAGAACAAGUAAA | 3266 | 4729-4749 | UUUUACUUGUUCUCAGAAAUCA | 3535 | 4727-4749 |
| | A | | | G | | |
| AD-1735357 | CCUACUUGUGAAAUACAUUU | 3267 | 4906-4926 | UAAAUGUAUUUCACAAGUAGGG | 3536 | 4904-4926 |
| | A | | | U | | |
| AD-1735358 | CAACUGGAAUUUUCCAAUUU | 3268 | 2297-2317 | UAAAUUGGAAAAUUCCAGUUGC | 3537 | 2295-2317 |
| | A | | | U | | |
| AD-1735359 | UCUAAUCCUGAUGAGAGUUA | 3269 | 2649-2669 | UUAACUCUCAUCAGGAUUAGAG | 3538 | 2647-2669 |
| | A | | | C | | |
| AD-1735360 | AGCCUCAUUAUCAAAAUUCU | 3270 | 1288-1308 | UAGAAUUUUGAUAAUGAGGCUU | 3539 | 1286-1308 |
| | A | | | A | | |
| AD-1735361 | AAUUUCUGAUGCUUUUUACU | 3271 | 4220-4240 | UAGUAAAAAGCAUCAGAAAUUG | 3540 | 4218-4240 |
| | A | | | A | | |
| AD-1735362 | UACCAUUAGAUGAAAUCUUA | 3272 | 5788-5808 | UUAAGAUUUCAUCUAAUGGUAC | 3541 | 5786-5808 |
| | A | | | A | | |
| AD-1735363 | CUGUGAAUUCUUCCAACCAUA | 3273 | 1816-1836 | UAUGGUUGGAAGAAUUCACAGG | 3542 | 1814-1836 |
| | | | | A | | |
| AD-1735364 | CUAAUACUCCAGAUUUUUAU | 3274 | 1948-1968 | UAUAAAAAUCUGGAGUAUUAGG | 3543 | 1946-1968 |
| | A | | | U | | |
| AD-1735365 | GGACACAUACAUUGAAUAAA | 3275 | 4550-4570 | UUUUAUUCAAUGUAUGUGUCCU | 3544 | 4548-4570 |
| | A | | | U | | |
| AD-1735366 | UUUGUCAUAAUGCUGCUUUG | 3276 | 3887-3907 | UCAAAGCAGCAUUAUGACAAAG | 3545 | 3885-3907 |
| | A | | | U | | |
| AD-1735367 | UUCAUCUUUCUGUUAAUUUC | 3277 | 4637-4657 | UGAAAUUAACAGAAAGAUGAAC | 3546 | 4635-4657 |
| | A | | | A | | |
| AD-1735368 | CAGUGAAACCAUUAAUUUUC | 3278 | 5028-5048 | UGAAAAUUAAUGGUUUCACUGU | 3547 | 5026-5048 |
| | A | | | G | | |
| AD-1735369 | UAACUGCAAGACUGAUUUCU | 3279 | 4716-4736 | UAGAAAUCAGUCUUGCAGUUAG | 3548 | 4714-4736 |
| | A | | | G | | |
| AD-1735370 | AGAAUUCCUCCUUACCUCAAA | 3280 | 3547-3567 | UUUGAGGUAAGGAGGAAUUCUC | 3549 | 3545-3567 |
| | | | | C | | |
| AD-1735371 | UAUUACAGAUCUUACUUUGU | 3281 | 5830-5850 | UACAAAGUAAGAUCUGUAAUAU | 3550 | 5828-5850 |
| | A | | | G | | |
| AD-1735372 | UUAUCUGAAAGAUAUCAAUU | 3282 | 5111-5131 | UAAUUGAUAUCUUUCAGAUAAG | 3551 | 5109-5131 |
| | A | | | C | | |
| AD-1735373 | AGAUCUGUUCUGGAAAAUCA | 3283 | 2811-2831 | UUGAUUUUCCAGAACAGAUCUG | 3552 | 2809-2831 |
| | A | | | U | | |
| AD-1735374 | CCCAGAAUACAACUUCCUUCA | 3284 | 2867-2887 | UGAAGGAAGUUGUAUUCUGGGC | 3553 | 2865-2887 |
| | | | | G | | |
| AD-1735375 | GUUGAUCUUUCAGUGCUAAA | 3285 | 1323-1343 | UUUUAGCACUGAAAGAUCAACU | 3554 | 1321-1343 |
| | A | | | C | | |
| AD-1735376 | CUAAGCUUUAUUUAUUAGAC | 3286 | 5191-5211 | UGUCUAAUAAAUAAAGCUUAGU | 3555 | 5189-5211 |
| | A | | | G | | |
| AD-1735377 | AGCUUGGAAUCUAUAUCUUU | 3287 | 2843-2863 | UAAAGAUAUAGAUUCCAAGCUG | 3556 | 2841-2863 |
| | A | | | A | | |
| AD-1735378 | UUAUUAUUUCUGAAGCCUAA | 3288 | 4699-4719 | UUUAGGCUUCAGAAAUAAUAAG | 3557 | 4697-4719 |
| | A | | | U | | |
| AD-1735379 | CCUUUGUUGUAUUUAACAAA | 3289 | 4187-4207 | UUUUGUUAAAUACAACAAAGGU | 3558 | 4185-4207 |
| | A | | | C | | |
| AD-1735380 | GCUCUCUAACUAAUCGAUCAA | 3290 | 1852-1872 | UUGAUCGAUUAGUUAGAGAGCC | 3559 | 1850-1872 |
| | | | | A | | |
| AD-1735381 | AAAGACUACAUGACAGAAAU | 3291 | 4666-4686 | UAUUUCUGUCAUGUAGUCUUUA | 3560 | 4664-4686 |
| | A | | | G | | |
| AD-1735382 | CAGGAUUCGUAUUUUUAAAG | 3292 | 4418-4438 | UCUUUAAAAAUACGAAUCCUGA | 3561 | 4416-4438 |
| | A | | | G | | |
| AD-1735383 | CCUUGCUAAGUAAUUGACAU | 3293 | 5659-5679 | UAUGUCAAUUACUUAGCAAGGG | 3562 | 5657-5679 |
| | A | | | U | | |
| AD-1735384 | CCUAUGCACUUUCACAGGAAA | 3294 | 3778-3798 | UUUCCUGUGAAAGUGCAUAGGA | 3563 | 3776-3798 |
| | | | | G | | |
| AD-1735385 | AUGCUGAAAUAUGUUUCAAC | 3295 | 2019-2039 | UGUUGAAACAUAUUUCAGCAUU | 3564 | 2017-2039 |
| | A | | | U | | |
| AD-1735386 | CCUUUUAAUUCAAAUCUACU | 3296 | 1710-1730 | UAGUAGAUUUGAAUUAAAAGGC | 3565 | 1708-1730 |
| | A | | | C | | |
| AD-1735387 | ACUGGACUUAUUGGAUUUAA | 3297 | 5577-5597 | UUUAAAUCCAAUAAGUCCAGUA | 3566 | 5575-5597 |
| | A | | | U | | |
| AD-1735388 | CAUCUGAAUCAGAUGGUACA | 3298 | 2038-2058 | UUGUACCAUCUGAUUCAGAUGU | 3567 | 2036-2058 |
| | A | | | U | | |
| AD-1735389 | AAUUCUGAUAGCAAUCUGUG | 3299 | 1980-2000 | UCACAGAUUGCUAUCAGAAUUU | 3568 | 1978-2000 |
| | A | | | C | | |
| AD-1735390 | UCAAGUCAAGGAUGCUAUCU | 3300 | 1683-1703 | UAGAUAGCAUCCUUGACUUGAA | 3569 | 1681-1703 |
| | A | | | A | | |
| AD-1735391 | UCACUGAAAACCACAAGAUA | 3301 | 3466-3486 | UUAUCUUGUGGUUUUCAGUGAG | 3570 | 3464-3486 |
| | A | | | G | | |
| AD-1735392 | ACCUGUUGAACAGUCUUCAA | 3302 | 1616-1636 | UUUGAAGACUGUUCAACAGGUA | 3571 | 1614-1636 |
| | A | | | G | | |
| AD-1735393 | UCAAGUGACAUAUUUCAGUU | 3303 | 3957-3977 | UAACUGAAAUAUGUCACUUGAG | 3572 | 3955-3977 |
| | A | | | U | | |
| AD-1735394 | UCAGGAACUUCAGGAUUGCU | 3304 | 1596-1616 | UAGCAAUCCUGAAGUUCCUGAG | 3573 | 1594-1616 |
| | A | | | C | | |
| AD-1735395 | ACGGAGUAUUAGUAGCUUAA | 3305 | 1412-1432 | UUUAAGCUACUAAUACUCCGUA | 3574 | 1410-1432 |
| | A | | | G | | |
| AD-1735396 | AUUGUCAAUGAAUUUUAUGA | 3306 | 3135-3155 | UUCAUAAAAUUCAUUGACAAUG | 3575 | 3133-3155 |
| | A | | | C | | |
| AD-1735397 | GAACAGCAAACAAAUAUUGA | 3307 | 2214-2234 | UUCAAUAUUUGUUUGCUGUUCA | 3576 | 2212-2234 |
| | A | | | C | | |
| AD-1735398 | UGAACUAUUUUCGUGCAUUA | 3308 | 2434-2454 | UUAAUGCACGAAAAUAGUUCAU | 3577 | 2432-2454 |
| | A | | | A | | |
| AD-1735399 | ACUGGAAUACUUAUUUUUCA | 3309 | 4751-4771 | UUGAAAAAUAAGUAUUCCAGUU | 3578 | 4749-4771 |
| | A | | | C | | |
| AD-1735400 | UUAUCUUGCAGAGAUCUCUG | 3310 | 1918-1938 | UCAGAGAUCUCUGCAAGAUAAC | 3579 | 1916-1938 |
| | A | | | G | | |
| AD-1735401 | GAAGUGUUAGAAUUUUUGAU | 3311 | 5142-5162 | UAUCAAAAAUUCUAACACUUCC | 3580 | 5140-5162 |
| | A | | | A | | |
| AD-1735402 | UCAAUUUAAGAUCUCUGGAA | 3312 | 5125-5145 | UUUCCAGAGAUCUUAAAUUGAU | 3581 | 5123-5145 |
| | A | | | A | | |
| AD-1735403 | UGGACCAUAUUUACAAUGUU | 3313 | 4856-4876 | UAACAUUGUAAAUAUGGUCCAU | 3582 | 4854-4876 |
| | A | | | U | | |
| AD-1735404 | UAGUCAUUGAAGCAAUCCUU | 3314 | 2926-2946 | UAAGGAUUGCUUCAAUGACUAA | 3583 | 2924-2946 |
| | A | | | A | | |
| AD-1735405 | AGCUGUUUAGGUUUAACAAU | 3315 | 4353-4373 | UAUUGUUAAACCUAAACAGCUU | 3584 | 4351-4373 |
| | A | | | U | | |
| AD-1735406 | CAAGGCAAAUGAUGUAAAUA | 3316 | 2993-3013 | UUAUUUACAUCAUUUGCCUUGG | 3585 | 2991-3013 |
| | A | | | C | | |
| AD-1735407 | AUCCAAUUACUACUGGAAAC | 3317 | 5603-5623 | UGUUUCCAGUAGUAAUUGGAUU | 3586 | 5601-5623 |
| | A | | | U | | |
| AD-1735408 | ACUUACAAUAUAUAUAUCCU | 3318 | 5328-5348 | UAGGAUAUAUAUAUUGUAAGUG | 3587 | 5326-5348 |
| | A | | | C | | |
| AD-1735409 | AUGCAGUUUGGUAUCUGACA | 3319 | 2512-2532 | UUGUCAGAUACCAAACUGCAUG | 3588 | 2510-2532 |
| | A | | | U | | |
| AD-1735410 | CCUUAUUUCAAGACACUGGU | 3320 | 2377-2397 | UACCAGUGUCUUGAAAUAAGGU | 3589 | 2375-2397 |
| | A | | | A | | |
| AD-1735411 | GAAGGGAUUUAUUCUUUAGU | 3321 | 4373-4393 | UACUAAAGAAUAAAUCCCUUCA | 3590 | 4371-4393 |
| | A | | | U | | |
| AD-1735412 | AUCCUUGCUACGGAUCUUAA | 3322 | 2940-2960 | UUUAAGAUCCGUAGCAAGGAUU | 3591 | 2938-2960 |
| | A | | | G | | |
| AD-1735413 | ACUGACUAUCCCGAAGCAAAA | 3323 | 1727-1747 | UUUUGCUUCGGGAUAGUCAGUA | 3592 | 1725-1747 |
| | | | | G | | |
| AD-1735414 | UGUAUUUCUGCUAUUAUUUC | 3324 | 5390-5410 | UGAAAUAAUAGCAGAAAUACAC | 3593 | 5388-5410 |
| | A | | | U | | |
| AD-1735415 | UGUAACAAUAACUAGCCUAA | 3325 | 5680-5700 | UUUAGGCUAGUUAUUGUUACAU | 3594 | 5678-5700 |
| | A | | | A | | |
| AD-1735416 | GUUGUUUUUACUCUAGCUCA | 3326 | 4400-4420 | UUGAGCUAGAGUAAAAACAACA | 3595 | 4398-4420 |
| | A | | | A | | |
| AD-1735417 | CAGGCAGUUUUAUACAAUGA | 3327 | 2790-2810 | UUCAUUGUAUAAAACUGCCUGA | 3596 | 2788-2810 |
| | A | | | G | | |
| AD-1735418 | AGGAUUCUCAGUCAGGUUAU | 3328 | 2352-2372 | UAUAACCUGACUGAGAAUCCUU | 3597 | 2350-2372 |
| | A | | | C | | |
| AD-1735419 | AUUUUCCAUCAUGUUUAAGU | 3329 | 5371-5391 | UACUUAAACAUGAUGGAAAAUG | 3598 | 5369-5391 |
| | A | | | U | | |
| AD-1735420 | UGCCACUUUUGUUACCAUUG | 3330 | 5238-5258 | UCAAUGGUAACAAAAGUGGCAG | 3599 | 5236-5258 |
| | A | | | C | | |
| AD-1735421 | ACUUGCUAGUGUGUGGAUAU | 3331 | 4882-4902 | UAUAUCCACACACUAGCAAGUU | 3600 | 4880-4902 |
| | A | | | U | | |
| AD-1735422 | AAUCUUACUUGAGAAAUUGC | 3332 | 5801-5821 | UGCAAUUUCUCAAGUAAGAUUU | 3601 | 5799-5821 |
| | A | | | C | | |
| AD-1735423 | AGUUCGAGACUUGCAUUUGA | 3333 | 3101-3121 | UUCAAAUGCAAGUCUCGAACUU | 3602 | 3099-3121 |
| | A | | | U | | |
| AD-1735424 | CCAUAGAAUUUCCUGAUACU | 3334 | 1873-1893 | UAGUAUCAGGAAAUUCUAUGGG | 3603 | 1871-1893 |
| | A | | | U | | |
| AD-1735425 | AGGUAAUUCCUUUAGAAUAU | 3335 | 5050-5070 | UAUAUUCUAAAGGAAUUACCUU | 3604 | 5048-5070 |
| | A | | | G | | |
| AD-1735426 | CUUAUGGAAACUCAACAAGA | 3336 | 2118-2138 | UUCUUGUUGAGUUUCCAUAAGU | 3605 | 2116-2138 |
| | A | | | U | | |
| AD-1735427 | AAGCAGCAUUUUAAAUUACG | 3337 | 5554-5574 | UCGUAAUUUAAAAUGCUGCUUU | 3606 | 5552-5574 |
| | A | | | U | | |
| AD-1735428 | UGACUCUCAACUGACCAUUCA | 3338 | 3699-3719 | UGAAUGGUCAGUUGAGAGUCAG | 3607 | 3697-3719 |
| | | | | C | | |
| AD-1735429 | AGGUUUAUCCAUGAAGGACU | 3339 | 4161-4181 | UAGUCCUUCAUGGAUAAACCUA | 3608 | 4159-4181 |
| | A | | | G | | |
| AD-1735430 | ACCAAGAAUUUGGCAUUUCA | 3340 | 1666-1686 | UUGAAAUGCCAAAUUCUUGGUG | 3609 | 1664-1686 |
| | A | | | A | | |
| AD-1735431 | UAUCUAGAAUAUUUGGCUUA | 3341 | 5094-5114 | UUAAGCCAAAUAUUCUAGAUAA | 3610 | 5092-5114 |
| | A | | | G | | |
| AD-1735432 | CUCCUGCAGAAUACAUACAAA | 3342 | 5412-5432 | UUUGUAUGUAUUCUGCAGGAGA | 3611 | 5410-5432 |
| | | | | G | | |
| AD-1735433 | UCACACCAUUUCCUGGAUUUA | 3343 | 1474-1494 | UAAAUCCAGGAAAUGGUGUGAG | 3612 | 1472-1494 |
| | | | | A | | |
| AD-1735434 | AUGUGGAAUUCAAGCGCUUU | 3344 | 2899-2919 | UAAAGCGCUUGAAUUCCACAUG | 3613 | 2897-2919 |
| | A | | | A | | |
| AD-1735435 | AUUGUGUAUUAUCUACUAUG | 3345 | 4240-4260 | UCAUAGUAGAUAAUACACAAUA | 3614 | 4238-4260 |
| | A | | | G | | |
| AD-1735436 | AAUGGAGUAAUGAAAAUGAU | 3346 | 3025-3045 | UAUCAUUUUCAUUACUCCAUUC | 3615 | 3023-3045 |
| | A | | | U | | |
| AD-1735437 | ACUGGUUUAUUGGAAAUAUU | 3347 | 2391-2411 | UAAUAUUUCCAAUAAACCAGUG | 3616 | 2389-2411 |
| | A | | | U | | |
| AD-1735438 | CAAGUGUAUGUGUAUAAAGU | 3348 | 5429-5449 | UACUUUAUACACAUACACUUGU | 3617 | 5427-5449 |
| | A | | | A | | |
| AD-1735439 | UACAAGCAUGCAUAUUGAGA | 3349 | 5457-5477 | UUCUCAAUAUGCAUGCUUGUAC | 3618 | 5455-5477 |
| | A | | | A | | |
| AD-1735440 | UGUGUCUUUUAUGUCUUUGA | 3350 | 4036-4056 | UUCAAAGACAUAAAAGACACAC | 3619 | 4034-4056 |
| | A | | | U | | |
| AD-1735441 | AUUUUCUCGCAGAAUUCAAU | 3351 | 2971-2991 | UAUUGAAUUCUGCGAGAAAAUC | 3620 | 2969-2991 |
| | A | | | A | | |
| AD-1735442 | UAGCGACAGUUUGAGUAAAA | 3352 | 3609-3629 | UUUUUACUCAAACUGUCGCUAU | 3621 | 3607-3629 |
| | A | | | U | | |
| AD-1735443 | CUGGUGUAUGAAUACUUUGU | 3353 | 3872-3892 | UACAAAGUAUUCAUACACCAGU | 3622 | 3870-3892 |
| | A | | | G | | |
| AD-1735444 | GUGAUAAGUGGCUAACAGAA | 3354 | 2182-2202 | UUUCUGUUAGCCACUUAUCACC | 3623 | 2180-2202 |
| | A | | | U | | |
| AD-1735445 | GUAGGCUAAUAUUUUCUAUU | 3355 | 4962-4982 | UAAUAGAAAAUAUUAGCCUACC | 3624 | 4960-4982 |
| | A | | | A | | |
| AD-1735446 | CCUCUUCAUCCUCGACUGUCA | 3356 | 1089-1109 | UGACAGUCGAGGAUGAAGAGGC | 3625 | 1087-1109 |
| | | | | G | | |
| AD-1735447 | AAUUGCCAUAAGCCAUAUUA | 3357 | 5815-5835 | UUAAUAUGGCUUAUGGCAAUUU | 3626 | 5813-5835 |
| | A | | | C | | |
| AD-1735448 | GCGAAUAUUUUGUCAGCUAA | 3358 | 3437-3457 | UUUAGCUGACAAAAUAUUCGCC | 3627 | 3435-3457 |
| | A | | | G | | |
| AD-1735449 | AUCCAGUGUUGCCUUUCUGA | 3359 | 4093-4113 | UUCAGAAAGGCAACACUGGAUU | 3628 | 4091-4113 |
| | A | | | C | | |
| AD-1735450 | AAAGUCUAUGCCUGUCUAAA | 3360 | 4068-4088 | UUUUAGACAGGCAUAGACUUUU | 3629 | 4066-4088 |
| | A | | | C | | |
| AD-1735451 | GGACAUCAGAAGUUUGAAUU | 3361 | 4298-4318 | UAAUUCAAACUUCUGAUGUCCA | 3630 | 4296-4318 |
| | A | | | U | | |
| AD-1735452 | GGCGAAUUGCUUAUAUUUCU | 3362 | 2617-2637 | UAGAAAUAUAAGCAAUUCGCCC | 3631 | 2615-2637 |
| | A | | | A | | |
| AD-1735453 | CACACUAUGUGUAAACCAGU | 3363 | 5261-5281 | UACUGGUUUACACAUAGUGUGU | 3632 | 5259-5281 |
| | A | | | G | | |
| AD-1735454 | CAGGUGAAGAAGAAAACAUU | 3364 | 2077-2097 | UAAUGUUUUCUUCUUCACCUGA | 3633 | 2075-2097 |
| | A | | | U | | |
| AD-1735455 | GUGAGCUCUUAUUUUUCACU | 3365 | 3914-3934 | UAGUGAAAAAUAAGAGCUCACU | 3634 | 3912-3934 |
| | A | | | A | | |
| AD-1735456 | AAAGCUGAUGGGAAUAAACU | 3366 | 3519-3539 | UAGUUUAUUCCCAUCAGCUUUA | 3635 | 3517-3539 |
| | A | | | C | | |
| AD-1735457 | GUGUCGUUAGGAGAAACUGC | 3367 | 1164-1184 | UGCAGUUUCUCCUAACGACACG | 3636 | 1162-1184 |
| | A | | | C | | |
| AD-1735458 | GGGACUUAAAACAAUGGUAU | 3368 | 1267-1287 | UAUACCAUUGUUUUAAGUCCCA | 3637 | 1265-1287 |
| | A | | | A | | |
| AD-1735459 | CUGAUGGUAGAAUUAACCAU | 3369 | 2596-2616 | UAUGGUUAAUUCUACCAUCAGA | 3638 | 2594-2616 |
| | A | | | A | | |
| AD-1735460 | ACUAUGUGUGUUUUAUUUCU | 3370 | 4254-4274 | UAGAAAUAAAACACACAUAGUA | 3639 | 4252-4274 |
| | A | | | G | | |
| AD-1735461 | CUGAUAAUUUAUAUUUGCAC | 3371 | 5310-5330 | UGUGCAAAUAUAAAUUAUCAGU | 3640 | 5308-5330 |
| | A | | | C | | |
| AD-1735462 | GUCCUUAAAUUAUUUAACCC | 3372 | 5641-5661 | UGGGUUAAAUAAUUUAAGGACU | 3641 | 5639-5661 |
| | A | | | A | | |
| AD-1735463 | CGCAAUAUGGUGUCAGAUCU | 3373 | 1350-1370 | UAGAUCUGACACCAUAUUGCGA | 3642 | 1348-1370 |
| | A | | | G | | |
| AD-1735464 | UUCGAAGAGCUGCUCUAAUC | 3374 | 2636-2656 | UGAUUAGAGCAGCUCUUCGAAG | 3643 | 2634-2656 |
| | A | | | A | | |
| AD-1735465 | UUUCUGCUGAGAGUAUUCAG | 3375 | 4269-4289 | UCUGAAUACUCUCAGCAGAAAU | 3644 | 4267-4289 |
| | A | | | A | | |
| AD-1735466 | UUGAACAGGAAGUAUCACUG | 3376 | 2230-2250 | UCAGUGAUACUUCCUGUUCAAU | 3645 | 2228-2250 |
| | A | | | A | | |
| AD-1735467 | UGCCUUGUAUUUCCAGAGAA | 3377 | 1228-1248 | UUUCUCUGGAAAUACAAGGCAA | 3646 | 1226-1248 |
| | A | | | C | | |
| AD-1735468 | GUUACUAUGGCAGUUGCAAA | 3378 | 1189-1209 | UUUUGCAACUGCCAUAGUAACU | 3647 | 1187-1209 |
| | A | | | G | | |
| AD-1735469 | AUGGCUGAAUUUUAAAGCUG | 3379 | 4338-4358 | UCAGCUUUAAAAUUCAGCCAUG | 3648 | 4336-4358 |
| | A | | | G | | |
| AD-1735470 | GUCCAUUCAUGGAUCGUUCU | 3380 | 3193-3213 | UAGAACGAUCCAUGAAUGGACU | 3649 | 3191-3213 |
| | A | | | G | | |
| AD-1735471 | UGUGUAACUCCUAUGAUGCU | 3381 | 3268-3288 | UAGCAUCAUAGGAGUUACACAG | 3650 | 3266-3288 |
| | A | | | G | | |
| AD-1735472 | UGGCAGUUUCCCACUCCUAUA | 3382 | 3763-3783 | UAUAGGAGUGGGAAACUGCCAG | 3651 | 3761-3783 |
| | | | | C | | |
| AD-1735473 | CCUCAAGCAGAUGAGAUUCA | 3383 | 3561-3581 | UUGAAUCUCAUCUGCUUGAGGU | 3652 | 3559-3581 |
| | A | | | A | | |
| AD-1735474 | AUGUUGUUUUCUCAAAAGCA | 3384 | 5539-5559 | UUGCUUUUGAGAAAACAACAUA | 3653 | 5537-5559 |
| | A | | | A | | |
| AD-1735475 | GAGGGAUUUCUGCUCAAUGC | 3385 | 4111-4131 | UGCAUUGAGCAGAAAUCCCUCA | 3654 | 4109-4131 |
| | A | | | G | | |
| AD-1735476 | GCCUUCUUCUUCCUCACCUGA | 3386 | 654-674 | UCAGGUGAGGAAGAAGAAGGCA | 3655 | 652-674 |
| | | | | C | | |
| AD-1735477 | UUGAGAUUGAAUCACAUUUC | 3387 | 5471-5491 | UGAAAUGUGAUUCAAUCUCAAU | 3656 | 5469-5491 |
| | A | | | A | | |
| AD-1735478 | CUGUUCAGUGCUAUUCUCCCA | 3388 | 4137-4157 | UGGGAGAAUAGCACUGAACAGU | 3657 | 4135-4157 |
| | | | | G | | |
| AD-1735479 | CCUGGCUUACAGCAGAUCCAA | 3389 | 2544-2564 | UUGGAUCUGCUGUAAGCCAGGA | 3658 | 2542-2564 |
| | | | | A | | |
| AD-1735480 | GACAGCAGAAAAUUAUUUCA | 3390 | 2157-2177 | UUGAAAUAAUUUUCUGCUGUCU | 3659 | 2155-2177 |
| | A | | | U | | |
| AD-1735481 | UUGGGUUUCUUUAGUUUAUG | 3391 | 5522-5542 | UCAUAAACUAAAGAAACCCAAU | 3660 | 5520-5542 |
| | A | | | A | | |
| AD-1735482 | CACCAUGUAGGUCUCAGAAG | 3392 | 1770-1790 | UCUUCUGAGACCUACAUGGUGA | 3661 | 1768-1790 |
| | A | | | G | | |
| AD-1735483 | GUUGUGGAACAGGAAAUGAA | 3393 | 2569-2589 | UUUCAUUUCCUGUUCCACAACC | 3662 | 2567-2589 |
| | A | | | A | | |
| AD-1735484 | GGAGAACAUAUCAUAUUUUG | 3394 | 4988-5008 | UCAAAAUAUGAUAUGUUCUCCA | 3663 | 4986-5008 |
| | A | | | A | | |
| AD-1735485 | CAGCCAAAAUGAUGGCAAAA | 3395 | 4940-4960 | UUUUUGCCAUCAUUUUGGCUGC | 3664 | 4938-4960 |
| | A | | | U | | |
| AD-1735486 | CCAAUUUUUGAACUUGUAGA | 3396 | 2310-2330 | UUCUACAAGUUCAAAAAUUGGA | 3665 | 2308-2330 |
| | A | | | A | | |
| AD-1735487 | AUAGGAAUAGUUUGCCAACU | 3397 | 1558-1578 | UAGUUGGCAAACUAUUCCUAUU | 3666 | 1556-1578 |
| | A | | | U | | |
| AD-1735488 | UUUGAAUGUUUUGGAGAAAA | 3398 | 4051-4071 | UUUUUCUCCAAAACAUUCAAAG | 3667 | 4049-4071 |
| | A | | | A | | |
| AD-1735489 | GUCAAUAUUUAAUGAAUCAC | 3399 | 5172-5192 | UGUGAUUCAUUAAAUAUUGACA | 3668 | 5170-5192 |
| | A | | | C | | |
| AD-1735490 | UGAGAAUAUAUUAAAUGCAC | 3400 | 5764-5784 | UGUGCAUUUAAUAUAUUCUCAC | 3669 | 5762-5784 |
| | A | | | A | | |
| AD-1735491 | UAACAAGGGACUAAAUAGGA | 3401 | 1544-1564 | UUCCUAUUUAGUCCCUUGUUAA | 3670 | 1542-1564 |
| | A | | | G | | |
| AD-1735492 | GGCUUCUUCUUCCACCUCUGA | 3402 | 408-428 | UCAGAGGUGGAAGAAGAAGCCG | 3671 | 406-428 |
| | | | | C | | |
| AD-1735493 | UUGGAACAUGUGGUUAUCUU | 3403 | 4573-4593 | UAAGAUAACCACAUGUUCCAAU | 3672 | 4571-4593 |
| | A | | | U | | |
| AD-1735494 | CAGAGAACAGAUGAUUCUUU | 3404 | 1241-1261 | UAAAGAAUCAUCUGUUCUCUGG | 3673 | 1239-1261 |
| | A | | | A | | |
| AD-1735495 | CUGUUAUUUUAUUGGGUUUU | 3405 | 5499-5519 | UAAAACCCAAUAAAAUAACAGA | 3674 | 5497-5519 |
| | A | | | C | | |
| AD-1735496 | AUGAAGAAGCAAAUCUUGGU | 3406 | 3163-3183 | UACCAAGAUUUGCUUCUUCAUC | 3675 | 3161-3183 |
| | A | | | U | | |
| AD-1735497 | UUUGGAAUCAUGCAAUUUUG | 3407 | 5004-5024 | UCAAAAUUGCAUGAUUCCAAAA | 3676 | 5002-5024 |
| | A | | | U | | |
| AD-1735498 | CAAUGUUUUGUAAACUUGCU | 3408 | 4869-4889 | UAGCAAGUUUACAAAACAUUGU | 3677 | 4867-4889 |
| | A | | | A | | |
| AD-1735499 | GGAUCGUAAUAAUGGCAAAA | 3409 | 1640-1660 | UUUUUGCCAUUAUUACGAUCCC | 3678 | 1638-1660 |
| | A | | | A | | |
| AD-1735500 | GUGCUUUCUCAGGUUCCUGU | 3410 | 1435-1455 | UACAGGAACCUGAGAAAGCACC | 3679 | 1433-1455 |
| | A | | | C | | |
| AD-1735501 | AUGAUACUGAAAGUGGUGAU | 3411 | 3331-3351 | UAUCACCACUUUCAGUAUCAUU | 3680 | 3329-3351 |
| | A | | | A | | |
| AD-1735502 | UCACUGGACCUGAUUUUAGU | 3412 | 2244-2264 | UACUAAAAUCAGGUCCAGUGAU | 3681 | 2242-2264 |
| | A | | | A | | |
| AD-1735503 | GAACUUUUUGCUUUUAUGAA | 3413 | 3991-4011 | UUUCAUAAAAGCAAAAAGUUCC | 3682 | 3989-4011 |
| | A | | | U | | |
| AD-1735504 | GAACACAGCACAGAUUUGUU | 3414 | 4451-4471 | UAACAAAUCUGUGCUGUGUUCA | 3683 | 4449-4471 |
| | A | | | U | | |
| AD-1735505 | AUAUGGAAGGAAAUCGUAGA | 3415 | 3483-3503 | UUCUACGAUUUCCUUCCAUAUC | 3684 | 3481-3503 |
| | A | | | U | | |
| AD-1735506 | GCUAUCUAAAUGGGCCUUUU | 3416 | 1696-1716 | UAAAAGGCCCAUUUAGAUAGCA | 3685 | 1694-1716 |
| | A | | | U | | |
| AD-1735507 | CAAAACCACCAAGAAGGAAA | 3417 | 3409-3429 | UUUUCCUUCUUGGUGGUUUUGG | 3686 | 3407-3429 |
| | A | | | A | | |
| AD-1735508 | CCACAGAUGUGCUACAUGCAA | 3418 | 2497-2517 | UUGCAUGUAGCACAUCUGUGGC | 3687 | 2495-2517 |
| | | | | A | | |
| AD-1735509 | AAAGUCAUACAUGUACAAGC | 3419 | 5444-5464 | UGCUUGUACAUGUAUGACUUUA | 3688 | 5442-5464 |
| | A | | | U | | |
| AD-1735510 | UAGAAGCAGAAGAGGAUAAU | 3420 | 3313-3333 | UAUUAUCCUCUUCUGCUUCUAA | 3689 | 3311-3333 |
| | A | | | C | | |
| AD-1735511 | CGUGUGUUCGCACAGCUUGA | 3421 | 611-631 | UUCAAGCUGUGCGAACACACGC | 3690 | 609-631 |
| | A | | | U | | |
| AD-1735512 | GCUGGUGUUUUGUCCAGUCU | 3422 | 1791-1811 | UAGACUGGACAAAACACCAGCU | 3691 | 1789-1811 |
| | A | | | C | | |
| AD-1735513 | AUGGAAAACAAUCUAAAUCC | 3423 | 3390-3410 | UGGAUUUAGAUUGUUUUCCAUU | 3692 | 3388-3410 |
| | A | | | U | | |
| AD-1735514 | CAUUAGAAAAUGGCUAUCGA | 3424 | 2449-2469 | UUCGAUAGCCAUUUUCUAAUGC | 3693 | 2447-2469 |
| | A | | | A | | |
| AD-1735515 | AUGGACAGAAGGCAUUGUCA | 3425 | 3122-3142 | UUGACAAUGCCUUCUGUCCAUU | 3694 | 3120-3142 |
| | A | | | U | | |
| AD-1735516 | CAGAUUGCUGCAGUGGAAAA | 3426 | 2056-2076 | UUUUUCCACUGCAGCAAUCUGU | 3695 | 2054-2076 |
| | A | | | A | | |
| AD-1735517 | AGGUGGAAAUGGAGUUGAUC | 3427 | 1310-1330 | UGAUCAACUCCAUUUCCACCUC | 3696 | 1308-1330 |
| | A | | | C | | |
| AD-1735518 | CUGGGCAAUGCACCUAAUACA | 3428 | 1935-1955 | UGUAUUAGGUGCAUUGCCCAGA | 3697 | 1933-1955 |
| | | | | G | | |
| AD-1735519 | AUUUCCCUCUGCCUAAAGACA | 3429 | 4652-4672 | UGUCUUUAGGCAGAGGGAAAUU | 3698 | 4650-4672 |
| | | | | A | | |
| AD-1735520 | CGCUUCUGCAACGUGGAGCUA | 3430 | 429-449 | UAGCUCCACGUUGCAGAAGCGG | 3699 | 427-449 |
| | | | | C | | |
| AD-1735521 | AGAUUCAUAUUGUAUUUCCC | 3431 | 4014-4034 | UGGGAAAUACAAUAUGAAUCUA | 3700 | 4012-4034 |
| | A | | | U | | |
| AD-1735522 | AGGACAAAUGCAUUUCUAGU | 3432 | 2754-2774 | UACUAGAAAUGCAUUUGUCCUC | 3701 | 2752-2774 |
| | A | | | C | | |
| AD-1735523 | UUAGACGUGUUGAGUGAGUG | 3433 | 5205-5225 | UCACUCACUCAACACGUCUAAU | 3702 | 5203-5225 |
| | A | | | A | | |
| AD-1735524 | CUGUGGACAUCAAAUGCUGA | 3434 | 2006-2026 | UUCAGCAUUUGAUGUCCACAGC | 3703 | 2004-2026 |
| | A | | | U | | |
| AD-1735525 | AGGCCUUAAUACUGUGAGAG | 3435 | 3729-3749 | UCUCUCACAGUAUUAAGGCCUG | 3704 | 3727-3749 |
| | A | | | U | | |
| AD-1735526 | AGAAAUCAUUGCCUAGUGUU | 3436 | 3672-3692 | UAACACUAGGCAAUGAUUUCUG | 3705 | 3670-3692 |
| | A | | | C | | |
| AD-1735527 | UGAGUGCUGAGUUCCUUGCU | 3437 | 5219-5239 | UAGCAAGGAACUCAGCACUCAC | 3706 | 5217-5239 |
| | A | | | U | | |
| AD-1735528 | GCCAUGCAUGAUUAUGAUCA | 3438 | 2727-2747 | UUGAUCAUAAUCAUGCAUGGCA | 3707 | 2725-2747 |
| | A | | | G | | |
| AD-1735529 | UGCUGCUGGUUUGCUACCAG | 3439 | 3284-3304 | UCUGGUAGCAAACCAGCAGCAU | 3708 | 3282-3304 |
| | A | | | C | | |
| AD-1735530 | CAGAUGAAGAGGAAUAGCGA | 3440 | 3595-3615 | UUCGCUAUUCCUCUUCAUCUGC | 3709 | 3593-3615 |
| | A | | | C | | |
| AD-1735531 | AUCCAGGUGCAUCAAUUUCU | 3441 | 4207-4227 | UAGAAAUUGAUGCACCUGGAUU | 3710 | 4205-4227 |
| | A | | | U | | |
| AD-1735532 | AAUAAGCCAAGCGUUAUCUU | 3442 | 1905-1925 | UAAGAUAACGCUUGGCUUAUUA | 3711 | 1903-1925 |
| | A | | | A | | |
| AD-1735533 | GAGAGGAUCCUUGCUCUGCU | 3443 | 3744-3764 | UAGCAGAGCAAGGAUCCUCUCA | 3712 | 3742-3764 |
| | A | | | C | | |
| AD-1735534 | AUGAUGAAGACGGUGAAGAA | 3444 | 3349-3369 | UUUCUUCACCGUCUUCAUCAUC | 3713 | 3347-3369 |
| | A | | | A | | |
| AD-1735535 | UCGUAAGUGUAAGAAAGAAU | 3445 | 5743-5763 | UAUUCUUUCUUACACUUACGAA | 3714 | 5741-5763 |
| | A | | | U | | |
| AD-1735536 | AUCUGUGUAACAGCUGUGGA | 3446 | 1993-2013 | UUCCACAGCUGUUACACAGAUU | 3715 | 1991-2013 |
| | A | | | G | | |
| AD-1735537 | AUUUAAAAUUCCCACUCAACA | 3447 | 2408-2428 | UGUUGAGUGGGAAUUUUAAAUA | 3716 | 2406-2428 |
| | | | | U | | |
| AD-1735538 | GACCAGUGUCUACUGGCUCUA | 3448 | 1837-1857 | UAGAGCCAGUAGACACUGGUCC | 3717 | 1835-1857 |
| | | | | A | | |
| AD-1735539 | CGCUUUCGUUUUUUAGUCAU | 3449 | 2913-2933 | UAUGACUAAAAAACGAAAGCGC | 3718 | 2911-2933 |
| | A | | | U | | |
| AD-1735540 | UCCGUGUUGACCCAUGUUGCA | 3450 | 3829-3849 | UGCAACAUGGGUCAACACGGAU | 3719 | 3827-3849 |
| | | | | G | | |
| AD-1735541 | AUACAGAAGAUGAAGAAAUG | 3451 | 3373-3393 | UCAUUUCUUCAUCUUCUGUAUC | 3720 | 3371-3393 |
| | A | | | U | | |
| AD-1735542 | UCUCCCGAGCUGAAACUUAAA | 3452 | 5719-5739 | UUUAAGUUUCAGCUCGGGAGAG | 3721 | 5717-5739 |
| | | | | A | | |
| AD-1735543 | UGCAUCAAACUGGCAGAUAU | 3453 | 3066-3086 | UAUAUCUGCCAGUUUGAUGCAC | 3722 | 3064-3086 |
| | A | | | A | | |
| AD-1735544 | UUGCAAAAUAUUCAGGAGAC | 3454 | 1202-1222 | UGUCUCCUGAAUAUUUUGCAAC | 3723 | 1200-1222 |
| | A | | | U | | |
| AD-1735545 | ACAAUCGUAUACAUGCCACAA | 3455 | 2482-2502 | UUGUGGCAUGUAUACGAUUGUG | 3724 | 2480-2502 |
| | | | | A | | |
| AD-1735546 | AAUAGAAAAGAUGAGCAACU | 3456 | 2282-2302 | UAGUUGCUCAUCUUUUCUAUUA | 3725 | 2280-2302 |
| | A | | | A | | |
| AD-1735547 | UAUCUUAUGUUCCAUGGCUG | 3457 | 4325-4345 | UCAGCCAUGGAACAUAAGAUAA | 3726 | 4323-4345 |
| | A | | | G | | |
| AD-1735548 | CUACGUGAAGAGCUGCGUGA | 3458 | 362-382 | UUCACGCAGCUCUUCACGUAGC | 3727 | 360-382 |
| | A | | | C | | |
| AD-1735549 | GAAUUGAUGGCUCUAUACGU | 3459 | 2700-2720 | UACGUAUAGAGCCAUCAAUUCU | 3728 | 2698-2720 |
| | A | | | A | | |
| AD-1735550 | UUCAGGUAAUUGAAGAGGCA | 3460 | 3577-3597 | UUGCCUCUUCAAUUACCUGAAU | 3729 | 3575-3597 |
| | A | | | C | | |
| AD-1735551 | AAGGACUGAGUGACCUUUGU | 3461 | 4174-4194 | UACAAAGGUCACUCAGUCCUUC | 3730 | 4172-4194 |
| | A | | | A | | |
| AD-1735552 | CGAGAGUCUGAGGAACGGCU | 3462 | 344-364 | UAGCCGUUCCUCAGACUCUCGG | 3731 | 342-364 |
| | A | | | G | | |
| AD-1735553 | AUUCUUUGGGAUUGGGACUU | 3463 | 1254-1274 | UAAGUCCCAAUCCCAAAGAAUC | 3732 | 1252-1274 |
| | A | | | A | | |
| AD-1735554 | UACCAGGUCAGUGGUUAGAA | 3464 | 3298-3318 | UUUCUAACCACUGACCUGGUAG | 3733 | 3296-3318 |
| | A | | | C | | |
| AD-1735555 | AUUCAGGUUUGCCAUGGACA | 3465 | 4283-4303 | UUGUCCAUGGCAAACCUGAAUA | 3734 | 4281-4303 |
| | A | | | C | | |
| AD-1735556 | GCAGCUAAACUGGUCCUGGA | 3466 | 34-54 | UUCCAGGACCAGUUUAGCUGCC | 3735 | 32-54 |
| | A | | | G | | |
| AD-1735557 | AGUAAUGGCAUAGAAUGGAG | 3467 | 3012-3032 | UCUCCAUUCUAUGCCAUUACUA | 3736 | 3010-3032 |
| | A | | | U | | |
| AD-1735558 | AAUUGCAAGAAAAUGGACCA | 3468 | 4843-4863 | UUGGUCCAUUUUCUUGCAAUUA | 3737 | 4841-4863 |
| | A | | | U | | |
| AD-1735559 | CGGCACUGCGUUCUGGUGCUA | 3469 | 921-941 | UAGCACCAGAACGCAGUGCCGG | 3738 | 919-941 |
| | | | | A | | |
| AD-1735560 | AGUCAUAUUGAAGAAGCCCA | 3470 | 3633-3653 | UUGGGCUUCUUCAAUAUGACUU | 3739 | 3631-3653 |
| | A | | | U | | |
| AD-1735561 | UCAUCAUGCUGCGUCAGCUUA | 3471 | 2828-2848 | UAAGCUGACGCAGCAUGAUGAU | 3740 | 2826-2848 |
| | | | | U | | |
| AD-1735562 | AAAUGAUCGCCUCUUGGUAU | 3472 | 3038-3058 | UAUACCAAGAGGCGAUCAUUUU | 3741 | 3036-3058 |
| | A | | | C | | |
| AD-1735563 | AUUUGAAGAUAUAAAGAGCA | 3473 | 4922-4942 | UUGCUCUUUAUAUCUUCAAAUG | 3742 | 4920-4942 |
| | A | | | U | | |
| AD-1735564 | GGUCUCCUUCACCAGCCUCGA | 3474 | 965-985 | UCGAGGCUGGUGAAGGAGACCC | 3743 | 963-985 |
| | | | | A | | |
| AD-1735565 | GAAACAGAGAAGAAAGACAG | 3475 | 2142-2162 | UCUGUCUUUCUUCUCUGUUUCC | 3744 | 2140-2162 |
| | A | | | U | | |
| AD-1735566 | UUCUAGUGGCUACAAAUGCC | 3476 | 2767-2787 | UGGCAUUUGUAGCCACUAGAAA | 3745 | 2765-2787 |
| | A | | | U | | |
| AD-1735567 | GGAGAGAAAUCAGGAAGGAU | 3477 | 2337-2357 | UAUCCUUCCUGAUUUCUCUCCC | 3746 | 2335-2357 |
| | A | | | A | | |
| AD-1735568 | AUAUAGUGAGCCCUAAAGGA | 3478 | 4533-4553 | UUCCUUUAGGGCUCACUAUAUU | 3747 | 4531-4553 |
| | A | | | U | | |
| AD-1735569 | GCUGCCUUUGUCCUCGCCCUA | 3479 | 528-548 | UAGGGCGAGGACAAAGGCAGCC | 3748 | 526-548 |
| | | | | A | | |
| AD-1735570 | GGCAAAAGACCUCACCAAGAA | 3480 | 1653-1673 | UUCUUGGUGAGGUCUUUUGCCA | 3749 | 1651-1673 |
| | | | | U | | |
| AD-1735571 | UUCAGCAUCGCCUGUGCCUUA | 3481 | 639-659 | UAAGGCACAGGCGAUGCUGAAG | 3750 | 637-659 |
| | | | | A | | |
| AD-1735572 | GCUAAAUGAGGCUCGCAAUA | 3482 | 1337-1357 | UUAUUGCGAGCCUCAUUUAGCA | 3751 | 1335-1357 |
| | A | | | C | | |
| AD-1735573 | CAGCUUCGUCUGGUGGGUCU | 3483 | 950-970 | UAGACCCACCAGACGAAGCUGG | 3752 | 948-970 |
| | A | | | C | | |
| AD-1735574 | AUAUGGUAUUGGCAUGCAGU | 3484 | 5066-5086 | UACUGCAUGCCAAUACCAUAUU | 3753 | 5064-5086 |
| | A | | | C | | |
| AD-1735575 | AAACUGCAGGUGGAGAAUUC | 3485 | 3534-3554 | UGAAUUCUCCACCUGCAGUUUA | 3754 | 3532-3554 |
| | A | | | U | | |
| AD-1735576 | UAUGGCUGCCUGUCUUCAAA | 3486 | 2667-2687 | UUUUGAAGACAGGCAGCCAUAA | 3755 | 2665-2687 |
| | A | | | C | | |
| AD-1735577 | CUGUUCUGAAAUAGAGGACC | 3487 | 1499-1519 | UGGUCCUCUAUUUCAGAACAGG | 3756 | 1497-1519 |
| | A | | | G | | |
| AD-1735578 | UCUAUUUUUAAAGCCGCUGG | 3488 | 5898-5918 | UCCAGCGGCUUUAAAAAUAGAA | 3757 | 5896-5918 |
| | A | | | A | | |
| AD-1735579 | GCUGAGGAGAAGCUCAGGAA | 3489 | 1583-1603 | UUUCCUGAGCUUCUCCUCAGCU | 3758 | 1581-1603 |
| | A | | | G | | |
| AD-1735580 | CAAGCCUUCCACCACAAGUCA | 3490 | 1381-1401 | UGACUUGUGGUGGAAGGCUUGG | 3759 | 1379-1401 |
| | | | | A | | |
| AD-1735581 | UUCAGUUACCAAAGUGGCCA | 3491 | 3970-3990 | UUGGCCACUUUGGUAACUGAAA | 3760 | 3968-3990 |
| | A | | | U | | |
| AD-1735582 | CACCUGCUUCCUCACCCGGAA | 3492 | 668-688 | UUCCGGGUGAGGAAGCAGGUGA | 3761 | 666-688 |
| | | | | G | | |
| AD-1735583 | UUUUAUCACCCACAUAGUGG | 3493 | 3242-3262 | UCCACUAUGUGGGUGAUAAAAG | 3762 | 3240-3262 |
| | A | | | A | | |
| AD-1735584 | AGGAGACCGUCGUUGCCUUG | 3494 | 1215-1235 | UCAAGGCAACGACGGUCUCCUG | 3763 | 1213-1235 |
| | A | | | A | | |
| AD-1735585 | AUGGCCCAGCAAAAGUUCGA | 3495 | 3088-3108 | UUCGAACUUUUGCUGGGCCAUU | 3764 | 3086-3108 |
| | A | | | U | | |
| AD-1735586 | AGACGCCAAAGCCAUGCGGUA | 3496 | 290-310 | UACCGCAUGGCUUUGGCGUCUC | 3765 | 288-310 |
| | | | | G | | |
| AD-1735587 | CAGCUAAUGCACCACCUCACA | 3497 | 3450-3470 | UGUGAGGUGGUGCAUUAGCUGA | 3766 | 3448-3470 |
| | | | | C | | |
| AD-1735588 | GGAAAAGCAGACGGCGAAUA | 3498 | 3424-3444 | UUAUUCGCCGUCUGCUUUUCCU | 3767 | 3422-3444 |
| | A | | | U | | |
| AD-1735589 | GCUUUGCUGGGUAGUGAGCU | 3499 | 3901-3921 | UAGCUCACUACCCAGCAAAGCA | 3768 | 3899-3921 |
| | A | | | G | | |

TABLE 13
Modified Sense and Antisense Strand Sequences of PDE3B dsRNA Agents Comprising an Unsaturated C22 Hydrocarbon Chain
Conjugated to Position 6 on the Sense Strand, Counting from the 5′-end of the Sense Strand

| | | SEQ | | SEQ | | SEQ |
|---|---|---|---|---|---|---|
| Duplex | | ID | | ID | mRNA | ID |
| Name | Sense Sequence 5′ to 3 | NO: | Antisense Sequence 5′ to 3′ | NO: | Target Sequence 5′ to 3′ | NO: |
| AD- | ususccu(Uda)CfuUfCfAfucuugauc | 3769 | VPusUfsgauCfaAfGfaugaAfgAfaggaas | 4038 | ACUUCCUUCUUCAUCUUGA | 4307 |
| 1735321 | sasa | | gsu | | UCAU | |
| AD- | csascuc(Uda)AfaAfAfCfugauguuc | 3770 | VPusUfsgaaCfaUfCfaguuUfuAfgagugs | 4039 | UUCACUCUAAAACUGAUGU | 4308 |
| 1735322 | sasa | | asa | | UCAU | |
| AD- | uscsuuc(Uda)GfuAfUfCfacugacuc | 3771 | VPusUfsgagUfcAfGfugauAfcAfgaagas | 4040 | CAUCUUCUGUAUCACUGAC | 4309 |
| 1735323 | sasa | | usg | | UCAC | |
| AD- | csasgau(Cda)UfuCfUfGfacugaucc | 3772 | VPusUfsggaUfcAfGfucagAfaGfaucugs | 4041 | GUCAGAUCUUCUGACUGAU | 4310 |
| 1735324 | sasa | | asc | | CCAA | |
| AD- | usgsaca(Cda)AfuUfUfUfucuccaua | 3773 | VPusUfsuauGfgAfGfaaaaAfuGfugucas | 4042 | ACUGACACAUUUUUCUCCA | 4311 |
| 1735325 | sasa | | gsu | | UAAA | |
| AD- | gsgsaaa(Cda)UfcAfUfUfuuuacaua | 3774 | VPusUfsuauGfuAfAfaaauGfaGfuuuccs | 4043 | CUGGAAACUCAUUUUUACA | 4312 |
| 1735326 | sasa | | asg | | UAAU | |
| AD- | asuscag(Cda)AfaCfUfUfagaaauuc | 3775 | VPusAfsgaaUfuUfCfuaagUfuGfcugaus | 4044 | UUAUCAGCAACUUAGAAAU | 4313 |
| 1735327 | susa | | asa | | UCUG | |
| AD- | csusuau(Ada)UfuUfCfUfcuagagua | 3776 | VPusGfsuacUfcUfAfgagaAfaUfauaags | 4045 | UCCUUAUAUUUCUCUAGAG | 4314 |
| 1735328 | scsa | | gsa | | UACA | |
| AD- | asascuc(Cda)AfaGfAfAfucuuuuau | 3777 | VPusGfsauaAfaAfGfauucUfuGfgaguus | 4046 | AAAACUCCAAGAAUCUUUU | 4315 |
| 1735329 | scsa | | usu | | AUCA | |
| AD- | csusuca(Ada)AfcAfUfUfccugcauu | 3778 | VPusUfsaauGfcAfGfgaauGfuUfugaags | 4047 | GUCUUCAAACAUUCCUGCA | 4316 |
| 1735330 | sasa | | asc | | UUAG | |
| AD- | uscsguu(Cda)UfuCfUfCfcucaacua | 3779 | VPusCfsuagUfuGfAfggagAfaGfaacgas | 4048 | GAUCGUUCUUCUCCUCAAC | 4317 |
| 1735331 | sgsa | | usc | | UAGC | |
| AD- | asasaga(Uda)UfaAfUfCfcucucaca | 3780 | VPusGfsuguGfaGfAfggauUfaAfucuuu | 4049 | CCAAAGAUUAAUCCUCUCA | 4318 |
| 1735332 | scsa | | sgsg | | CACC | |
| AD- | ascsuaa(Cda)CfuCfAfUfcaaagaua | 3781 | VPusGfsuauCfuUfUfgaugAfgGfuuagu | 4050 | AAACUAACCUCAUCAAAGA | 4319 |
| 1735333 | scsa | | susu | | UACA | |
| AD- | csascca(Cda)UfuAfUfUfacuaauaa | 3782 | VPusUfsuuaUfuAfGfuaauAfaGfuggug | 4051 | CCCACCACUUAUUACUAAU | 4320 |
| 1735334 | sasa | | sgsg | | AAAA | |
| AD- | usgsgua(Cda)UfaGfAfAfauauucu | 3783 | VPusAfsaagAfaUfAfuuucUfaGfuaccas | 4052 | GCUGGUACUAGAAAUAUUC | 4321 |
| 1735335 | ususa | | gsc | | UUUU | |
| AD- | gscsugu(Cda)AfuUfUfGfuucacuc | 3784 | VPusUfsagaGfuGfAfacaaAfuGfacagcs | 4053 | UAGCUGUCAUUUGUUCACU | 4322 |
| 1735336 | usasa | | usa | | CUAA | |
| AD- | ascsuca(Ada)CfaAfUfUfuaugaacu | 3785 | VPusUfsaguUfcAfUfaaauUfgUfugagus | 4054 | CCACUCAACAAUUUAUGAA | 4323 |
| 1735337 | sasa | | gsg | | CUAU | |
| AD- | asgsuau(Gda)AfcUfCfAfuuaauaga | 3786 | VPusUfsucuAfuUfAfaugaGfuCfauacus | 4055 | AGAGUAUGACUCAUUAAUA | 4324 |
| 1735338 | sasa | | csu | | GAAA | |
| AD- | csusgcu(Gda)AfuUfUfUfcuuaauaa | 3787 | VPusCfsuuaUfuAfAfgaaaAfuCfagcags | 4056 | UACUGCUGAUUUUCUUAAU | 4325 |
| 1735339 | sgsa | | usa | | AAGC | |
| AD- | usasucu(Uda)UfaGfAfUfccacaucu | 3788 | VPusAfsagaUfgUfGfgaucUfaAfagauas | 4057 | GUUAUCUUUAGAUCCACAU | 4326 |
| 1735340 | susa | | asc | | CUUA | |
| AD- | csgsaga(Cda)AfuUfCfCfuuaucaca | 3789 | VPusUfsuguGfaUfAfaggaAfuGfucucg | 4058 | AUCGAGACAUUCCUUAUCA | 4327 |
| 1735341 | sasa | | sasu | | CAAU | |
| AD- | usascug(Ada)AfuCfAfGfauuaauu | 3790 | VPusGfsaaaUfuAfAfucugAfuUfcaguas | 4059 | GUUACUGAAUCAGAUUAAU | 4328 |
| 1735342 | uscsa | | asc | | UUCU | |
| AD- | csusuac(Uda)UfaAfAfUfccuucacu | 3791 | VPusCfsaguGfaAfGfgauuUfaAfguaags | 4060 | CCCUUACUUAAAUCCUUCA | 4329 |
| 1735343 | sgsa | | gsg | | CUGG | |
| AD- | uscsgaa(Ada)GfaAfUfCfauucaaac | 3792 | VPusAfsguuUfgAfAfugauUfcUfuucga | 4061 | UCUCGAAAGAAUCAUUCAA | 4330 |
| 1735344 | susa | | sgsa | | ACUU | |
| AD- | asasaug(Ada)CfcUfAfUfcacuacuu | 3793 | VPusUfsaagUfaGfUfgauaGfgUfcauuus | 4062 | AGAAAUGACCUAUCACUAC | 4331 |
| 1735345 | sasa | | csu | | UUAU | |
| AD- | csasagu(Cda)AfuUfUfCfcucucuac | 3794 | VPusCfsguaGfaGfAfggaaAfuGfacuugs | 4063 | CACAAGUCAUUUCCUCUCU | 4332 |
| 1735346 | sgsa | | usg | | ACGG | |
| AD- | asusgca(Gda)UfuUfCfUfuacuuauc | 3795 | VPusAfsgauAfaGfUfaagaAfaCfugcaus | 4064 | GCAUGCAGUUUCUUACUUA | 4333 |
| 1735347 | susa | | gsc | | UCUA | |
| AD- | uscsaau(Gda)CfaAfUfAfcacuguuc | 3796 | VPusUfsgaaCfaGfUfguauUfgCfauugas | 4065 | GCUCAAUGCAAUACACUGU | 4334 |
| 1735348 | sasa | | gsc | | UCAG | |
| AD- | uscsuau(Gda)UfaCfCfUfacugacac | 3797 | VPusUfsgugUfcAfGfuaggUfaCfauagas | 4066 | UAUCUAUGUACCUACUGAC | 4335 |
| 1735349 | sasa | | usa | | ACAU | |
| AD- | usgsaau(Uda)CfcAfGfUfcuuaucuu | 3798 | VPusUfsaagAfuAfAfgacuGfgAfauucas | 4067 | UUUGAAUUCCAGUCUUAUC | 4336 |
| 1735350 | sasa | | asa | | UUAU | |
| AD- | ususgcu(Gda)UfaAfUfAfccaaaacu | 3799 | VPusUfsaguUfuUfGfguauUfaCfagcaas | 4068 | AUUUGCUGUAAUACCAAAA | 4337 |
| 1735351 | sasa | | asu | | CUAA | |
| AD- | gsgsauc(Ada)CfuUfCfUfuucaaauc | 3800 | VPusUfsgauUfuGfAfaagaAfgUfgauccs | 4069 | UUGGAUCACUUCUUUCAAA | 4338 |
| 1735352 | sasa | | asa | | UCAG | |
| AD- | gsasacu(Ada)GfaAfAfAfcuauucuu | 3801 | VPusUfsaagAfaUfAfguuuUfcUfaguucs | 4070 | AGGAACUAGAAAACUAUUC | 4339 |
| 1735353 | sasa | | csu | | UUAA | |
| AD- | ascsauu(Uda)CfcAfUfAfcugucugu | 3802 | VPusAfsacaGfaCfAfguauGfgAfaaugus | 4071 | UCACAUUUCCAUACUGUCU | 4340 |
| 1735354 | susa | | gsa | | GUUA | |
| AD- | usgsauc(Cda)UfuCfAfCfagugucaa | 3803 | VPusAfsuugAfcAfCfugugAfaGfgaucas | 4072 | UUUGAUCCUUCACAGUGUC | 4341 |
| 1735355 | susa | | asa | | AAUA | |
| AD- | gsasuuu(Cda)UfgAfGfAfacaaguaa | 3804 | VPusUfsuuaCfuUfGfuucuCfaGfaaaucs | 4073 | CUGAUUUCUGAGAACAAGU | 4342 |
| 1735356 | sasa | | asg | | AAAG | |
| AD- | cscsuac(Uda)UfgUfGfAfaauacauu | 3805 | VPusAfsaauGfuAfUfuucaCfaAfguaggs | 4074 | ACCCUACUUGUGAAAUACA | 4343 |
| 1735357 | susa | | gsu | | UUUG | |
| AD- | csasacu(Gda)GfaAfUfUfuuccaauu | 3806 | VPusAfsaauUfgGfAfaaauUfcCfaguugs | 4075 | AGCAACUGGAAUUUUCCAA | 4344 |
| 1735358 | susa | | csu | | UUUU | |
| AD- | uscsuaa(Uda)CfcUfGfAfugagaguu | 3807 | VPusUfsaacUfcUfCfaucaGfgAfuuagas | 4076 | GCUCUAAUCCUGAUGAGAG | 4345 |
| 1735359 | sasa | | gsc | | UUAU | |
| AD- | asgsccu(Cda)AfuUfAfUfcaaaauuc | 3808 | VPusAfsgaaUfuUfUfgauaAfuGfaggcus | 4077 | UAAGCCUCAUUAUCAAAAU | 4346 |
| 1735360 | susa | | usa | | UCUG | |
| AD- | asasuuu(Cda)UfgAfUfGfcuuuuua | 3809 | VPusAfsguaAfaAfAfgcauCfaGfaaauus | 4078 | UCAAUUUCUGAUGCUUUUU | 4347 |
| 1735361 | csusa | | gsa | | ACUA | |
| AD- | usascca(Uda)UfaGfAfUfgaaaucuu | 3810 | VPusUfsaagAfuUfUfcaucUfaAfugguas | 4079 | UGUACCAUUAGAUGAAAUC | 4348 |
| 1735362 | sasa | | csa | | UUAC | |
| AD- | csusgug(Ada)AfuUfCfUfuccaacca | 3811 | VPusAfsuggUfuGfGfaagaAfuUfcacags | 4080 | UCCUGUGAAUUCUUCCAAC | 4349 |
| 1735363 | susa | | gsa | | CAUG | |
| AD- | csusaau(Ada)CfuCfCfAfgauuuuua | 3812 | VPusAfsuaaAfaAfUfcuggAfgUfauuags | 4081 | ACCUAAUACUCCAGAUUUU | 4350 |
| 1735364 | susa | | gsu | | UAUC | |
| AD- | gsgsaca(Cda)AfuAfCfAfuugaauaa | 3813 | VPusUfsuuaUfuCfAfauguAfuGfugucc | 4082 | AAGGACACAUACAUUGAAU | 4351 |
| 1735365 | sasa | | susu | | AAAU | |
| AD- | ususugu(Cda)AfuAfAfUfgcugcuu | 3814 | VPusCfsaaaGfcAfGfcauuAfuGfacaaas | 4083 | ACUUUGUCAUAAUGCUGCU | 4352 |
| 1735366 | usgsa | | gsu | | UUGC | |
| AD- | ususcau(Cda)UfuUfCfUfguuaauu | 3815 | VPusGfsaaaUfuAfAfcagaAfaGfaugaas | 4084 | UGUUCAUCUUUCUGUUAAU | 4353 |
| 1735367 | uscsa | | csa | | UUCC | |
| AD- | csasgug(Ada)AfaCfCfAfuuaauuuu | 3816 | VPusGfsaaaAfuUfAfauggUfuUfcacugs | 4085 | CACAGUGAAACCAUUAAUU | 4354 |
| 1735368 | scsa | | usg | | UUCC | |
| AD- | usasacu(Gda)CfaAfGfAfcugauuuc | 3817 | VPusAfsgaaAfuCfAfgucuUfgCfaguuas | 4086 | CCUAACUGCAAGACUGAUU | 4355 |
| 1735369 | susa | | gsg | | UCUG | |
| AD- | asgsaau(Uda)CfcUfCfCfuuaccuca | 3818 | VPusUfsugaGfgUfAfaggaGfgAfauucu | 4087 | GGAGAAUUCCUCCUUACCU | 4356 |
| 1735370 | sasa | | scsc | | CAAG | |
| AD- | usasuua(Cda)AfgAfUfCfuuacuuu | 3819 | VPusAfscaaAfgUfAfagauCfuGfuaauas | 4088 | CAUAUUACAGAUCUUACUU | 4357 |
| 1735371 | gsusa | | usg | | UGUU | |
| AD- | ususauc(Uda)GfaAfAfGfauaucaau | 3820 | VPusAfsauuGfaUfAfucuuUfcAfgauaas | 4089 | GCUUAUCUGAAAGAUAUCA | 4358 |
| 1735372 | susa | | gsc | | AUUU | |
| AD- | asgsauc(Uda)GfuUfCfUfggaaaauc | 3821 | VPusUfsgauUfuUfCfcagaAfcAfgaucus | 4090 | ACAGAUCUGUUCUGGAAAA | 4359 |
| 1735373 | sasa | | gsu | | UCAU | |
| AD- | cscscag(Ada)AfuAfCfAfacuuccuu | 3822 | VPusGfsaagGfaAfGfuuguAfuUfcuggg | 4091 | CGCCCAGAAUACAACUUCC | 4360 |
| 1735374 | scsa | | scsg | | UUCU | |
| AD- | gsusuga(Uda)CfuUfUfCfagugcuaa | 3823 | VPusUfsuuaGfcAfCfugaaAfgAfucaacs | 4092 | GAGUUGAUCUUUCAGUGCU | 4361 |
| 1735375 | sasa | | usc | | AAAU | |
| AD- | csusaag(Cda)UfuUfAfUfuuauuaga | 3824 | VPusGfsucuAfaUfAfaauaAfaGfcuuags | 4093 | CACUAAGCUUUAUUUAUUA | 4362 |
| 1735376 | scsa | | usg | | GACG | |
| AD- | asgscuu(Gda)GfaAfUfCfuauaucuu | 3825 | VPusAfsaagAfuAfUfagauUfcCfaagcus | 4094 | UCAGCUUGGAAUCUAUAUC | 4363 |
| 1735377 | susa | | gsa | | UUUC | |
| AD- | ususauu(Ada)UfuUfCfUfgaagccua | 3826 | VPusUfsuagGfcUfUfcagaAfaUfaauaas | 4095 | ACUUAUUAUUUCUGAAGCC | 4364 |
| 1735378 | sasa | | gsu | | UAAC | |
| AD- | cscsuuu(Gda)UfuGfUfAfuuuaacaa | 3827 | VPusUfsuugUfuAfAfauacAfaCfaaaggs | 4096 | GACCUUUGUUGUAUUUAAC | 4365 |
| 1735379 | sasa | | usc | | AAAA | |
| AD- | gscsucu(Cda)UfaAfCfUfaaucgauc | 3828 | VPusUfsgauCfgAfUfuaguUfaGfagagcs | 4097 | UGGCUCUCUAACUAAUCGA | 4366 |
| 1735380 | sasa | | csa | | UCAC | |
| AD- | asasaga(Cda)UfaCfAfUfgacagaaa | 3829 | VPusAfsuuuCfuGfUfcaugUfaGfucuuu | 4098 | CUAAAGACUACAUGACAGA | 4367 |
| 1735381 | susa | | sasg | | AAUG | |
| AD- | csasgga(Uda)UfcGfUfAfuuuuuaaa | 3830 | VPusCfsuuuAfaAfAfauacGfaAfuccugs | 4099 | CUCAGGAUUCGUAUUUUUA | 4368 |
| 1735382 | sgsa | | asg | | AAGA | |
| AD- | cscsuug(Cda)UfaAfGfUfaauugaca | 3831 | VPusAfsuguCfaAfUfuacuUfaGfcaaggs | 4100 | ACCCUUGCUAAGUAAUUGA | 4369 |
| 1735383 | susa | | gsu | | CAUA | |
| AD- | cscsuau(Gda)CfaCfUfUfucacagga | 3832 | VPusUfsuccUfgUfGfaaagUfgCfauaggs | 4101 | CUCCUAUGCACUUUCACAG | 4370 |
| 1735384 | sasa | | asg | | GAAC | |
| AD- | asusgcu(Gda)AfaAfUfAfuguuuca | 3833 | VPusGfsuugAfaAfCfauauUfuCfagcaus | 4102 | AAAUGCUGAAAUAUGUUUC | 4371 |
| 1735385 | ascsa | | usu | | AACA | |
| AD- | cscsuuu(Uda)AfaUfUfCfaaaucuac | 3834 | VPusAfsguaGfaUfUfugaaUfuAfaaaggs | 4103 | GGCCUUUUAAUUCAAAUCU | 4372 |
| 1735386 | susa | | csc | | ACUG | |
| AD- | ascsugg(Ada)CfuUfAfUfuggauuu | 3835 | VPusUfsuaaAfuCfCfaauaAfgUfccagus | 4104 | AUACUGGACUUAUUGGAUU | 4373 |
| 1735387 | asasa | | asu | | UAAU | |
| AD- | csasucu(Gda)AfaUfCfAfgaugguac | 3836 | VPusUfsguaCfcAfUfcugaUfuCfagaugs | 4105 | AACAUCUGAAUCAGAUGGU | 4374 |
| 1735388 | sasa | | usu | | ACAG | |
| AD- | asasuuc(Uda)GfaUfAfGfcaaucugu | 3837 | VPusCfsacaGfaUfUfgcuaUfcAfgaauus | 4106 | GAAAUUCUGAUAGCAAUCU | 4375 |
| 1735389 | sgsa | | usc | | GUGU | |
| AD- | uscsaag(Uda)CfaAfGfGfaugcuauc | 3838 | VPusAfsgauAfgCfAfuccuUfgAfcuugas | 4107 | UUUCAAGUCAAGGAUGCUA | 4376 |
| 1735390 | susa | | asa | | UCUA | |
| AD- | uscsacu(Gda)AfaAfAfCfcacaagau | 3839 | VPusUfsaucUfuGfUfgguuUfuCfaguga | 4108 | CCUCACUGAAAACCACAAG | 4377 |
| 1735391 | sasa | | sgsg | | AUAU | |
| AD- | ascscug(Uda)UfgAfAfCfagucuuca | 3840 | VPusUfsugaAfgAfCfuguuCfaAfcaggus | 4109 | CUACCUGUUGAACAGUCUU | 4378 |
| 1735392 | sasa | | asg | | CAAG | |
| AD- | uscsaag(Uda)GfaCfAfUfauuucagu | 3841 | VPusAfsacuGfaAfAfuaugUfcAfcuugas | 4110 | ACUCAAGUGACAUAUUUCA | 4379 |
| 1735393 | susa | | gsu | | GUUA | |
| AD- | uscsagg(Ada)AfcUfUfCfaggauugc | 3842 | VPusAfsgcaAfuCfCfugaaGfuUfccugas | 4111 | GCUCAGGAACUUCAGGAUU | 4380 |
| 1735394 | susa | | gsc | | GCUA | |
| AD- | ascsgga(Gda)UfaUfUfAfguagcuua | 3843 | VPusUfsuaaGfcUfAfcuaaUfaCfuccgus | 4112 | CUACGGAGUAUUAGUAGCU | 4381 |
| 1735395 | sasa | | asg | | UAAU | |
| AD- | asusugu(Cda)AfaUfGfAfauuuuau | 3844 | VPusUfscauAfaAfAfuucaUfuGfacaaus | 4113 | GCAUUGUCAAUGAAUUUUA | 4382 |
| 1735396 | gsasa | | gsc | | UGAG | |
| AD- | gsasaca(Gda)CfaAfAfCfaaauauug | 3845 | VPusUfscaaUfaUfUfuguuUfgCfuguucs | 4114 | GUGAACAGCAAACAAAUAU | 4383 |
| 1735397 | sasa | | asc | | UGAA | |
| AD- | usgsaac(Uda)AfuUfUfUfcgugcau | 3846 | VPusUfsaauGfcAfCfgaaaAfuAfguucas | 4115 | UAUGAACUAUUUUCGUGCA | 4384 |
| 1735398 | usasa | | usa | | UUAG | |
| AD- | ascsugg(Ada)AfuAfCfUfuauuuuu | 3847 | VPusUfsgaaAfaAfUfaaguAfuUfccagus | 4116 | GAACUGGAAUACUUAUUUU | 4385 |
| 1735399 | csasa | | usc | | UCAU | |
| AD- | ususauc(Uda)UfgCfAfGfagaucucu | 3848 | VPusCfsagaGfaUfCfucugCfaAfgauaas | 4117 | CGUUAUCUUGCAGAGAUCU | 4386 |
| 1735400 | sgsa | | csg | | CUGG | |
| AD- | gsasagu(Gda)UfuAfGfAfauuuuug | 3849 | VPusAfsucaAfaAfAfuucuAfaCfacuucs | 4118 | UGGAAGUGUUAGAAUUUU | 4387 |
| 1735401 | asusa | | csa | | UGAUC | |
| AD- | uscsaau(Uda)UfaAfGfAfucucugga | 3850 | VPusUfsuccAfgAfGfaucuUfaAfauugas | 4119 | UAUCAAUUUAAGAUCUCUG | 4388 |
| 1735402 | sasa | | usa | | GAAG | |
| AD- | usgsgac(Cda)AfuAfUfUfuacaaugu | 3851 | VPusAfsacaUfuGfUfaaauAfuGfguccas | 4120 | AAUGGACCAUAUUUACAAU | 4389 |
| 1735403 | susa | | usu | | GUUU | |
| AD- | usasguc(Ada)UfuGfAfAfgcaauccu | 3852 | VPusAfsaggAfuUfGfcuucAfaUfgacuas | 4121 | UUUAGUCAUUGAAGCAAUC | 4390 |
| 1735404 | susa | | asa | | CUUG | |
| AD- | asgscug(Uda)UfuAfGfGfuuuaacaa | 3853 | VPusAfsuugUfuAfAfaccuAfaAfcagcus | 4122 | AAAGCUGUUUAGGUUUAAC | 4391 |
| 1735405 | susa | | usu | | AAUG | |
| AD- | csasagg(Cda)AfaAfUfGfauguaaau | 3854 | VPusUfsauuUfaCfAfucauUfuGfccuugs | 4123 | GCCAAGGCAAAUGAUGUAA | 4392 |
| 1735406 | sasa | | gsc | | AUAG | |
| AD- | asuscca(Ada)UfuAfCfUfacuggaaa | 3855 | VPusGfsuuuCfcAfGfuaguAfaUfuggau | 4124 | AAAUCCAAUUACUACUGGA | 4393 |
| 1735407 | scsa | | susu | | AACU | |
| AD- | ascsuua(Cda)AfaUfAfUfauauaucc | 3856 | VPusAfsggaUfaUfAfuauaUfuGfuaagus | 4125 | GCACUUACAAUAUAUAUAU | 4394 |
| 1735408 | susa | | gsc | | CCUG | |
| AD- | asusgca(Gda)UfuUfGfGfuaucugac | 3857 | VPusUfsgucAfgAfUfaccaAfaCfugcaus | 4126 | ACAUGCAGUUUGGUAUCUG | 4395 |
| 1735409 | sasa | | gsu | | ACAA | |
| AD- | cscsuua(Uda)UfuCfAfAfgacacugg | 3858 | VPusAfsccaGfuGfUfcuugAfaAfuaaggs | 4127 | UACCUUAUUUCAAGACACU | 4396 |
| 1735410 | susa | | usa | | GGUU | |
| AD- | gsasagg(Gda)AfuUfUfAfuucuuua | 3859 | VPusAfscuaAfaGfAfauaaAfuCfccuucs | 4128 | AUGAAGGGAUUUAUUCUUU | 4397 |
| 1735411 | gsusa | | asu | | AGUC | |
| AD- | asusccu(Uda)GfcUfAfCfggaucuua | 3860 | VPusUfsuaaGfaUfCfcguaGfcAfaggaus | 4129 | CAAUCCUUGCUACGGAUCU | 4398 |
| 1735412 | sasa | | usg | | UAAA | |
| AD- | ascsuga(Cda)UfaUfCfCfcgaagcaa | 3861 | VPusUfsuugCfuUfCfgggaUfaGfucagus | 4130 | CUACUGACUAUCCCGAAGC | 4399 |
| 1735413 | sasa | | asg | | AAAG | |
| AD- | usgsuau(Uda)UfcUfGfCfuauuauu | 3862 | VPusGfsaaaUfaAfUfagcaGfaAfauacas | 4131 | AGUGUAUUUCUGCUAUUAU | 4400 |
| 1735414 | uscsa | | csu | | UUCC | |
| AD- | usgsuaa(Cda)AfaUfAfAfcuagccua | 3863 | VPusUfsuagGfcUfAfguuaUfuGfuuacas | 4132 | UAUGUAACAAUAACUAGCC | 4401 |
| 1735415 | sasa | | usa | | UAAA | |
| AD- | gsusugu(Uda)UfuUfAfCfucuagcu | 3864 | VPusUfsgagCfuAfGfaguaAfaAfacaacs | 4133 | UUGUUGUUUUUACUCUAGC | 4402 |
| 1735416 | csasa | | asa | | UCAG | |
| AD- | csasggc(Ada)GfuUfUfUfauacaaug | 3865 | VPusUfscauUfgUfAfuaaaAfcUfgccugs | 4134 | CUCAGGCAGUUUUAUACAA | 4403 |
| 1735417 | sasa | | asg | | UGAC | |
| AD- | asgsgau(Uda)CfuCfAfGfucagguua | 3866 | VPusAfsuaaCfcUfGfacugAfgAfauccus | 4135 | GAAGGAUUCUCAGUCAGGU | 4404 |
| 1735418 | susa | | usc | | UAUG | |
| AD- | asusuuu(Cda)CfaUfCfAfuguuuaag | 3867 | VPusAfscuuAfaAfCfaugaUfgGfaaaaus | 4136 | ACAUUUUCCAUCAUGUUUA | 4405 |
| 1735419 | susa | | gsu | | AGUG | |
| AD- | usgscca(Cda)UfuUfUfGfuuaccauu | 3868 | VPusCfsaauGfgUfAfacaaAfaGfuggcas | 4137 | GCUGCCACUUUUGUUACCA | 4406 |
| 1735420 | sgsa | | gsc | | UUGU | |
| AD- | ascsuug(Cda)UfaGfUfGfuguggau | 3869 | VPusAfsuauCfcAfCfacacUfaGfcaagus | 4138 | AAACUUGCUAGUGUGUGGA | 4407 |
| 1735421 | asusa | | usu | | UAUG | |
| AD- | asasucu(Uda)AfcUfUfGfagaaauug | 3870 | VPusGfscaaUfuUfCfucaaGfuAfagauus | 4139 | GAAAUCUUACUUGAGAAAU | 4408 |
| 1735422 | scsa | | usc | | UGCC | |
| AD- | asgsuuc(Gda)AfgAfCfUfugcauuu | 3871 | VPusUfscaaAfuGfCfaaguCfuCfgaacus | 4140 | AAAGUUCGAGACUUGCAUU | 4409 |
| 1735423 | gsasa | | usu | | UGAA | |
| AD- | cscsaua(Gda)AfaUfUfUfccugauac | 3872 | VPusAfsguaUfcAfGfgaaaUfuCfuauggs | 4141 | ACCCAUAGAAUUUCCUGAU | 4410 |
| 1735424 | susa | | gsu | | ACUG | |
| AD- | asgsgua(Ada)UfuCfCfUfuuagaaua | 3873 | VPusAfsuauUfcUfAfaaggAfaUfuaccus | 4142 | CAAGGUAAUUCCUUUAGAA | 4411 |
| 1735425 | susa | | usg | | UAUG | |
| AD- | csusuau(Gda)GfaAfAfCfucaacaag | 3874 | VPusUfscuuGfuUfGfaguuUfcCfauaags | 4143 | AACUUAUGGAAACUCAACA | 4412 |
| 1735426 | sasa | | usu | | AGAA | |
| AD- | asasgca(Gda)CfaUfUfUfuaaauuac | 3875 | VPusCfsguaAfuUfUfaaaaUfgCfugcuus | 4144 | AAAAGCAGCAUUUUAAAUU | 4413 |
| 1735427 | sgsa | | usu | | ACGA | |
| AD- | usgsacu(Cda)UfcAfAfCfugaccauu | 3876 | VPusGfsaauGfgUfCfaguuGfaGfagucas | 4145 | GCUGACUCUCAACUGACCA | 4414 |
| 1735428 | scsa | | gsc | | UUCC | |
| AD- | asgsguu(Uda)AfuCfCfAfugaaggac | 3877 | VPusAfsgucCfuUfCfauggAfuAfaaccus | 4146 | CUAGGUUUAUCCAUGAAGG | 4415 |
| 1735429 | susa | | asg | | ACUG | |
| AD- | ascscaa(Gda)AfaUfUfUfggcauuuc | 3878 | VPusUfsgaaAfuGfCfcaaaUfuCfuuggus | 4147 | UCACCAAGAAUUUGGCAUU | 4416 |
| 1735430 | sasa | | gsa | | UCAA | |
| AD- | usasucu(Ada)GfaAfUfAfuuuggcu | 3879 | VPusUfsaagCfcAfAfauauUfcUfagauas | 4148 | CUUAUCUAGAAUAUUUGGC | 4417 |
| 1735431 | usasa | | asg | | UUAU | |
| AD- | csusccu(Gda)CfaGfAfAfuacauaca | 3880 | VPusUfsuguAfuGfUfauucUfgCfaggag | 4149 | CUCUCCUGCAGAAUACAUA | 4418 |
| 1735432 | sasa | | sasg | | CAAG | |
| AD- | uscsaca(Cda)CfaUfUfUfccuggauu | 3881 | VPusAfsaauCfcAfGfgaaaUfgGfugugas | 4150 | UCUCACACCAUUUCCUGGA | 4419 |
| 1735433 | susa | | gsa | | UUUU | |
| AD- | asusgug(Gda)AfaUfUfCfaagcgcuu | 3882 | VPusAfsaagCfgCfUfugaaUfuCfcacaus | 4151 | UCAUGUGGAAUUCAAGCGC | 4420 |
| 1735434 | susa | | gsa | | UUUC | |
| AD- | asusugu(Gda)UfaUfUfAfucuacua | 3883 | VPusCfsauaGfuAfGfauaaUfaCfacaaus | 4152 | CUAUUGUGUAUUAUCUACU | 4421 |
| 1735435 | usgsa | | asg | | AUGU | |
| AD- | asasugg(Ada)GfuAfAfUfgaaaauga | 3884 | VPusAfsucaUfuUfUfcauuAfcUfccauus | 4153 | AGAAUGGAGUAAUGAAAA | 4422 |
| 1735436 | susa | | csu | | UGAUC | |
| AD- | ascsugg(Uda)UfuAfUfUfggaaaua | 3885 | VPusAfsauaUfuUfCfcaauAfaAfccagus | 4154 | ACACUGGUUUAUUGGAAAU | 4423 |
| 1735437 | ususa | | gsu | | AUUU | |
| AD- | csasagu(Gda)UfaUfGfUfguauaaag | 3886 | VPusAfscuuUfaUfAfcacaUfaCfacuugs | 4155 | UACAAGUGUAUGUGUAUAA | 4424 |
| 1735438 | susa | | usa | | AGUC | |
| AD- | usascaa(Gda)CfaUfGfCfauauugag | 3887 | VPusUfscucAfaUfAfugcaUfgCfuuguas | 4156 | UGUACAAGCAUGCAUAUUG | 4425 |
| 1735439 | sasa | | csa | | AGAU | |
| AD- | usgsugu(Cda)UfuUfUfAfugucuuu | 3888 | VPusUfscaaAfgAfCfauaaAfaGfacacas | 4157 | AGUGUGUCUUUUAUGUCUU | 4426 |
| 1735440 | gsasa | | csu | | UGAA | |
| AD- | asusuuu(Cda)UfcGfCfAfgaauucaa | 3889 | VPusAfsuugAfaUfUfcugcGfaGfaaaaus | 4158 | UGAUUUUCUCGCAGAAUUC | 4427 |
| 1735441 | susa | | csa | | AAUG | |
| AD- | usasgcg(Ada)CfaGfUfUfugaguaaa | 3890 | VPusUfsuuuAfcUfCfaaacUfgUfcgcuas | 4159 | AAUAGCGACAGUUUGAGUA | 4428 |
| 1735442 | sasa | | usu | | AAAG | |
| AD- | csusggu(Gda)UfaUfGfAfauacuuu | 3891 | VPusAfscaaAfgUfAfuucaUfaCfaccags | 4160 | CACUGGUGUAUGAAUACUU | 4429 |
| 1735443 | gsusa | | usg | | UGUC | |
| AD- | gsusgau(Ada)AfgUfGfGfcuaacaga | 3892 | VPusUfsucuGfuUfAfgccaCfuUfaucacs | 4161 | AGGUGAUAAGUGGCUAACA | 4430 |
| 1735444 | sasa | | csu | | GAAG | |
| AD- | gsusagg(Cda)UfaAfUfAfuuuucua | 3893 | VPusAfsauaGfaAfAfauauUfaGfccuacs | 4162 | UGGUAGGCUAAUAUUUUCU | 4431 |
| 1735445 | ususa | | csa | | AUUA | |
| AD- | cscsucu(Uda)CfaUfCfCfucgacugu | 3894 | VPusGfsacaGfuCfGfaggaUfgAfagaggs | 4163 | CGCCUCUUCAUCCUCGACU | 4432 |
| 1735446 | scsa | | csg | | GUCC | |
| AD- | asasuug(Cda)CfaUfAfAfgccauauu | 3895 | VPusUfsaauAfuGfGfcuuaUfgGfcaauus | 4164 | GAAAUUGCCAUAAGCCAUA | 4433 |
| 1735447 | sasa | | usc | | UUAC | |
| AD- | gscsgaa(Uda)AfuUfUfUfgucagcua | 3896 | VPusUfsuagCfuGfAfcaaaAfuAfuucgcs | 4165 | CGGCGAAUAUUUUGUCAGC | 4434 |
| 1735448 | sasa | | csg | | UAAU | |
| AD- | asuscca(Gda)UfgUfUfGfccuuucug | 3897 | VPusUfscagAfaAfGfgcaaCfaCfuggaus | 4166 | GAAUCCAGUGUUGCCUUUC | 4435 |
| 1735449 | sasa | | usc | | UGAG | |
| AD- | asasagu(Cda)UfaUfGfCfcugucuaa | 3898 | VPusUfsuuaGfaCfAfggcaUfaGfacuuus | 4167 | GAAAAGUCUAUGCCUGUCU | 4436 |
| 1735450 | sasa | | usc | | AAAA | |
| AD- | gsgsaca(Uda)CfaGfAfAfguuugaau | 3899 | VPusAfsauuCfaAfAfcuucUfgAfuguccs | 4168 | AUGGACAUCAGAAGUUUGA | 4437 |
| 1735451 | susa | | asu | | AUUC | |
| AD- | gsgscga(Ada)UfuGfCfUfuauauuu | 3900 | VPusAfsgaaAfuAfUfaagcAfaUfucgccs | 4169 | UGGGCGAAUUGCUUAUAUU | 4438 |
| 1735452 | csusa | | csa | | UCUU | |
| AD- | csascac(Uda)AfuGfUfGfuaaaccag | 3901 | VPusAfscugGfuUfUfacacAfuAfgugug | 4170 | CACACACUAUGUGUAAACC | 4439 |
| 1735453 | susa | | susg | | AGUC | |
| AD- | csasggu(Gda)AfaGfAfAfgaaaacau | 3902 | VPusAfsaugUfuUfUfcuucUfuCfaccugs | 4171 | AUCAGGUGAAGAAGAAAAC | 4440 |
| 1735454 | susa | | asu | | AUUU | |
| AD- | gsusgag(Cda)UfcUfUfAfuuuuuca | 3903 | VPusAfsgugAfaAfAfauaaGfaGfcucacs | 4172 | UAGUGAGCUCUUAUUUUUC | 4441 |
| 1735455 | csusa | | usa | | ACUG | |
| AD- | asasagc(Uda)GfaUfGfGfgaauaaac | 3904 | VPusAfsguuUfaUfUfcccaUfcAfgcuuus | 4173 | GUAAAGCUGAUGGGAAUAA | 4442 |
| 1735456 | susa | | asc | | ACUG | |
| AD- | gsusguc(Gda)UfuAfGfGfagaaacu | 3905 | VPusGfscagUfuUfCfuccuAfaCfgacacs | 4174 | GCGUGUCGUUAGGAGAAAC | 4443 |
| 1735457 | gscsa | | gsc | | UGCA | |
| AD- | gsgsgac(Uda)UfaAfAfAfcaauggua | 3906 | VPusAfsuacCfaUfUfguuuUfaAfgucccs | 4175 | UUGGGACUUAAAACAAUGG | 4444 |
| 1735458 | susa | | asa | | UAUA | |
| AD- | csusgau(Gda)GfuAfGfAfauuaacca | 3907 | VPusAfsuggUfuAfAfuucuAfcCfaucags | 4176 | UUCUGAUGGUAGAAUUAAC | 4445 |
| 1735459 | susa | | asa | | CAUG | |
| AD- | ascsuau(Gda)UfgUfGfUfuuuauuu | 3908 | VPusAfsgaaAfuAfAfaacaCfaCfauagus | 4177 | CUACUAUGUGUGUUUUAUU | 4446 |
| 1735460 | csusa | | asg | | UCUG | |
| AD- | csusgau(Ada)AfuUfUfAfuauuugc | 3909 | VPusGfsugcAfaAfUfauaaAfuUfaucags | 4178 | GACUGAUAAUUUAUAUUUG | 4447 |
| 1735461 | ascsa | | usc | | CACU | |
| AD- | gsusccu(Uda)AfaAfUfUfauuuaacc | 3910 | VPusGfsgguUfaAfAfuaauUfuAfaggacs | 4179 | UAGUCCUUAAAUUAUUUAA | 4448 |
| 1735462 | scsa | | usa | | CCCU | |
| AD- | csgscaa(Uda)AfuGfGfUfgucagauc | 3911 | VPusAfsgauCfuGfAfcaccAfuAfuugcgs | 4180 | CUCGCAAUAUGGUGUCAGA | 4449 |
| 1735463 | susa | | asg | | UCUU | |
| AD- | ususcga(Ada)GfaGfCfUfgcucuaau | 3912 | VPusGfsauuAfgAfGfcagcUfcUfucgaas | 4181 | UCUUCGAAGAGCUGCUCUA | 4450 |
| 1735464 | scsa | | gsa | | AUCC | |
| AD- | ususucu(Gda)CfuGfAfGfaguauuc | 3913 | VPusCfsugaAfuAfCfucucAfgCfagaaas | 4182 | UAUUUCUGCUGAGAGUAUU | 4451 |
| 1735465 | asgsa | | usa | | CAGG | |
| AD- | ususgaa(Cda)AfgGfAfAfguaucacu | 3914 | VPusCfsaguGfaUfAfcuucCfuGfuucaas | 4183 | UAUUGAACAGGAAGUAUCA | 4452 |
| 1735466 | sgsa | | usa | | CUGG | |
| AD- | usgsccu(Uda)GfuAfUfUfuccagaga | 3915 | VPusUfsucuCfuGfGfaaauAfcAfaggcas | 4184 | GUUGCCUUGUAUUUCCAGA | 4453 |
| 1735467 | sasa | | asc | | GAAC | |
| AD- | gsusuac(Uda)AfuGfGfCfaguugcaa | 3916 | VPusUfsuugCfaAfCfugccAfuAfguaacs | 4185 | CAGUUACUAUGGCAGUUGC | 4454 |
| 1735468 | sasa | | usg | | AAAA | |
| AD- | asusggc(Uda)GfaAfUfUfuuaaagcu | 3917 | VPusCfsagcUfuUfAfaaauUfcAfgccaus | 4186 | CCAUGGCUGAAUUUUAAAG | 4455 |
| 1735469 | sgsa | | gsg | | CUGU | |
| AD- | gsuscca(Uda)UfcAfUfGfgaucguuc | 3918 | VPusAfsgaaCfgAfUfccauGfaAfuggacs | 4187 | CAGUCCAUUCAUGGAUCGU | 4456 |
| 1735470 | susa | | usg | | UCUU | |
| AD- | usgsugu(Ada)AfcUfCfCfuaugaug | 3919 | VPusAfsgcaUfcAfUfaggaGfuUfacacas | 4188 | CCUGUGUAACUCCUAUGAU | 4457 |
| 1735471 | csusa | | gsg | | GCUG | |
| AD- | usgsgca(Gda)UfuUfCfCfcacuccua | 3920 | VPusAfsuagGfaGfUfgggaAfaCfugccas | 4189 | GCUGGCAGUUUCCCACUCC | 4458 |
| 1735472 | susa | | gsc | | UAUG | |
| AD- | cscsuca(Ada)GfcAfGfAfugagauuc | 3921 | VPusUfsgaaUfcUfCfaucuGfcUfugaggs | 4190 | UACCUCAAGCAGAUGAGAU | 4459 |
| 1735473 | sasa | | usa | | UCAG | |
| AD- | asusguu(Gda)UfuUfUfCfucaaaagc | 3922 | VPusUfsgcuUfuUfGfagaaAfaCfaacaus | 4191 | UUAUGUUGUUUUCUCAAAA | 4460 |
| 1735474 | sasa | | asa | | GCAG | |
| AD- | gsasggg(Ada)UfuUfCfUfgcucaau | 3923 | VPusGfscauUfgAfGfcagaAfaUfcccucs | 4192 | CUGAGGGAUUUCUGCUCAA | 4461 |
| 1735475 | gscsa | | asg | | UGCA | |
| AD- | gscscuu(Cda)UfuCfUfUfccucaccu | 3924 | VPusCfsaggUfgAfGfgaagAfaGfaaggcs | 4193 | GUGCCUUCUUCUUCCUCAC | 4462 |
| 1735476 | sgsa | | asc | | CUGC | |
| AD- | ususgag(Ada)UfuGfAfAfucacauu | 3925 | VPusGfsaaaUfgUfGfauucAfaUfcucaas | 4194 | UAUUGAGAUUGAAUCACAU | 4463 |
| 1735477 | uscsa | | usa | | UUCC | |
| AD- | csusguu(Cda)AfgUfGfCfuauucucc | 3926 | VPusGfsggaGfaAfUfagcaCfuGfaacags | 4195 | CACUGUUCAGUGCUAUUCU | 4464 |
| 1735478 | scsa | | usg | | CCCA | |
| AD- | cscsugg(Cda)UfuAfCfAfgcagaucc | 3927 | VPusUfsggaUfcUfGfcuguAfaGfccaggs | 4196 | UUCCUGGCUUACAGCAGAU | 4465 |
| 1735479 | sasa | | asa | | CCAC | |
| AD- | gsascag(Cda)AfgAfAfAfauuauuuc | 3928 | VPusUfsgaaAfuAfAfuuuuCfuGfcuguc | 4197 | AAGACAGCAGAAAAUUAUU | 4466 |
| 1735480 | sasa | | susu | | UCAG | |
| AD- | ususggg(Uda)UfuCfUfUfuaguuua | 3929 | VPusCfsauaAfaCfUfaaagAfaAfcccaas | 4198 | UAUUGGGUUUCUUUAGUUU | 4467 |
| 1735481 | usgsa | | usa | | AUGU | |
| AD- | csascca(Uda)GfuAfGfGfucucagaa | 3930 | VPusCfsuucUfgAfGfaccuAfcAfuggugs | 4199 | CUCACCAUGUAGGUCUCAG | 4468 |
| 1735482 | sgsa | | asg | | AAGA | |
| AD- | gsusugu(Gda)GfaAfCfAfggaaaug | 3931 | VPusUfsucaUfuUfCfcuguUfcCfacaacs | 4200 | UGGUUGUGGAACAGGAAAU | 4469 |
| 1735483 | asasa | | csa | | GAAA | |
| AD- | gsgsaga(Ada)CfaUfAfUfcauauuuu | 3932 | VPusCfsaaaAfuAfUfgauaUfgUfucuccs | 4201 | UUGGAGAACAUAUCAUAUU | 4470 |
| 1735484 | sgsa | | asa | | UUGG | |
| AD- | csasgcc(Ada)AfaAfUfGfauggcaaa | 3933 | VPusUfsuuuGfcCfAfucauUfuUfggcug | 4202 | AGCAGCCAAAAUGAUGGCA | 4471 |
| 1735485 | sasa | | scsu | | AAAU | |
| AD- | cscsaau(Uda)UfuUfGfAfacuuguag | 3934 | VPusUfscuaCfaAfGfuucaAfaAfauuggs | 4203 | UUCCAAUUUUUGAACUUGU | 4472 |
| 1735486 | sasa | | asa | | AGAA | |
| AD- | asusagg(Ada)AfuAfGfUfuugccaac | 3935 | VPusAfsguuGfgCfAfaacuAfuUfccuaus | 4204 | AAAUAGGAAUAGUUUGCCA | 4473 |
| 1735487 | susa | | usu | | ACUC | |
| AD- | ususuga(Ada)UfgUfUfUfuggagaa | 3936 | VPusUfsuuuCfuCfCfaaaaCfaUfucaaas | 4205 | UCUUUGAAUGUUUUGGAGA | 4474 |
| 1735488 | asasa | | gsa | | AAAG | |
| AD- | gsuscaa(Uda)AfuUfUfAfaugaauca | 3937 | VPusGfsugaUfuCfAfuuaaAfuAfuugacs | 4206 | GUGUCAAUAUUUAAUGAAU | 4475 |
| 1735489 | scsa | | asc | | CACU | |
| AD- | usgsaga(Ada)UfaUfAfUfuaaaugca | 3938 | VPusGfsugcAfuUfUfaauaUfaUfucucas | 4207 | UGUGAGAAUAUAUUAAAU | 4476 |
| 1735490 | scsa | | csa | | GCACA | |
| AD- | usasaca(Ada)GfgGfAfCfuaaauagg | 3939 | VPusUfsccuAfuUfUfagucCfcUfuguuas | 4208 | CUUAACAAGGGACUAAAUA | 4477 |
| 1735491 | sasa | | asg | | GGAA | |
| AD- | gsgscuu(Cda)UfuCfUfUfccaccucu | 3940 | VPusCfsagaGfgUfGfgaagAfaGfaagccs | 4209 | GCGGCUUCUUCUUCCACCU | 4478 |
| 1735492 | sgsa | | gsc | | CUGC | |
| AD- | ususgga(Ada)CfaUfGfUfgguuauc | 3941 | VPusAfsagaUfaAfCfcacaUfgUfuccaas | 4210 | AAUUGGAACAUGUGGUUAU | 4479 |
| 1735493 | ususa | | usu | | CUUU | |
| AD- | csasgag(Ada)AfcAfGfAfugauucu | 3942 | VPusAfsaagAfaUfCfaucuGfuUfcucugs | 4211 | UCCAGAGAACAGAUGAUUC | 4480 |
| 1735494 | ususa | | gsa | | UUUG | |
| AD- | csusguu(Ada)UfuUfUfAfuuggguu | 3943 | VPusAfsaaaCfcCfAfauaaAfaUfaacagsa | 4212 | GUCUGUUAUUUUAUUGGGU | 4481 |
| 1735495 | ususa | | sc | | UUUA | |
| AD- | asusgaa(Gda)AfaGfCfAfaaucuugg | 3944 | VPusAfsccaAfgAfUfuugcUfuCfuucaus | 4213 | AGAUGAAGAAGCAAAUCUU | 4482 |
| 1735496 | susa | | csu | | GGUC | |
| AD- | ususugg(Ada)AfuCfAfUfgcaauuu | 3945 | VPusCfsaaaAfuUfGfcaugAfuUfccaaas | 4214 | AUUUUGGAAUCAUGCAAUU | 4483 |
| 1735497 | usgsa | | asu | | UUGC | |
| AD- | csasaug(Uda)UfuUfGfUfaaacuugc | 3946 | VPusAfsgcaAfgUfUfuacaAfaAfcauugs | 4215 | UACAAUGUUUUGUAAACUU | 4484 |
| 1735498 | susa | | usa | | GCUA | |
| AD- | gsgsauc(Gda)UfaAfUfAfauggcaaa | 3947 | VPusUfsuuuGfcCfAfuuauUfaCfgauccs | 4216 | UGGGAUCGUAAUAAUGGCA | 4485 |
| 1735499 | sasa | | csa | | AAAG | |
| AD- | gsusgcu(Uda)UfcUfCfAfgguuccu | 3948 | VPusAfscagGfaAfCfcugaGfaAfagcacs | 4217 | GGGUGCUUUCUCAGGUUCC | 4486 |
| 1735500 | gsusa | | csc | | UGUA | |
| AD- | asusgau(Ada)CfuGfAfAfaguggug | 3949 | VPusAfsucaCfcAfCfuuucAfgUfaucaus | 4218 | UAAUGAUACUGAAAGUGGU | 4487 |
| 1735501 | asusa | | usa | | GAUG | |
| AD- | uscsacu(Gda)GfaCfCfUfgauuuuag | 3950 | VPusAfscuaAfaAfUfcaggUfcCfagugas | 4219 | UAUCACUGGACCUGAUUUU | 4488 |
| 1735502 | susa | | usa | | AGUA | |
| AD- | gsasacu(Uda)UfuUfGfCfuuuuaug | 3951 | VPusUfsucaUfaAfAfagcaAfaAfaguucs | 4220 | AGGAACUUUUUGCUUUUAU | 4489 |
| 1735503 | asasa | | csu | | GAAA | |
| AD- | gsasaca(Cda)AfgCfAfCfagauuugu | 3952 | VPusAfsacaAfaUfCfugugCfuGfuguucs | 4221 | AUGAACACAGCACAGAUUU | 4490 |
| 1735504 | susa | | asu | | GUUA | |
| AD- | asusaug(Gda)AfaGfGfAfaaucguag | 3953 | VPusUfscuaCfgAfUfuuccUfuCfcauaus | 4222 | AGAUAUGGAAGGAAAUCGU | 4491 |
| 1735505 | sasa | | csu | | AGAG | |
| AD- | gscsuau(Cda)UfaAfAfUfgggccuu | 3954 | VPusAfsaaaGfgCfCfcauuUfaGfauagcs | 4223 | AUGCUAUCUAAAUGGGCCU | 4492 |
| 1735506 | ususa | | asu | | UUUA | |
| AD- | csasaaa(Cda)CfaCfCfAfagaaggaas | 3955 | VPusUfsuucCfuUfCfuuggUfgGfuuuug | 4224 | UCCAAAACCACCAAGAAGG | 4493 |
| 1735507 | asa | | sgsa | | AAAA | |
| AD- | cscsaca(Gda)AfuGfUfGfcuacaugc | 3956 | VPusUfsgcaUfgUfAfgcacAfuCfuguggs | 4225 | UGCCACAGAUGUGCUACAU | 4494 |
| 1735508 | sasa | | csa | | GCAG | |
| AD- | asasagu(Cda)AfuAfCfAfuguacaag | 3957 | VPusGfscuuGfuAfCfauguAfuGfacuuu | 4226 | AUAAAGUCAUACAUGUACA | 4495 |
| 1735509 | scsa | | sasu | | AGCA | |
| AD- | usasgaa(Gda)CfaGfAfAfgaggauaa | 3958 | VPusAfsuuaUfcCfUfcuucUfgCfuucuas | 4227 | GUUAGAAGCAGAAGAGGAU | 4496 |
| 1735510 | susa | | asc | | AAUG | |
| AD- | csgsugu(Gda)UfuCfGfCfacagcuug | 3959 | VPusUfscaaGfcUfGfugcgAfaCfacacgs | 4228 | AGCGUGUGUUCGCACAGCU | 4497 |
| 1735511 | sasa | | csu | | UGAG | |
| AD- | gscsugg(Uda)GfuUfUfUfguccagu | 3960 | VPusAfsgacUfgGfAfcaaaAfcAfccagcs | 4229 | GAGCUGGUGUUUUGUCCAG | 4498 |
| 1735512 | csusa | | usc | | UCUG | |
| AD- | asusgga(Ada)AfaCfAfAfucuaaauc | 3961 | VPusGfsgauUfuAfGfauugUfuUfuccau | 4230 | AAAUGGAAAACAAUCUAAA | 4499 |
| 1735513 | scsa | | susu | | UCCA | |
| AD- | csasuua(Gda)AfaAfAfUfggcuaucg | 3962 | VPusUfscgaUfaGfCfcauuUfuCfuaaugs | 4231 | UGCAUUAGAAAAUGGCUAU | 4500 |
| 1735514 | sasa | | csa | | CGAG | |
| AD- | asusgga(Cda)AfgAfAfGfgcauugu | 3963 | VPusUfsgacAfaUfGfccuuCfuGfuccaus | 4232 | AAAUGGACAGAAGGCAUUG | 4501 |
| 1735515 | csasa | | usu | | UCAA | |
| AD- | csasgau(Uda)GfcUfGfCfaguggaaa | 3964 | VPusUfsuuuCfcAfCfugcaGfcAfaucugs | 4233 | UACAGAUUGCUGCAGUGGA | 4502 |
| 1735516 | sasa | | usa | | AAAU | |
| AD- | asgsgug(Gda)AfaAfUfGfgaguuga | 3965 | VPusGfsaucAfaCfUfccauUfuCfcaccus | 4234 | GGAGGUGGAAAUGGAGUU | 4503 |
| 1735517 | uscsa | | CSC | | GAUCU | |
| AD- | csusggg(Cda)AfaUfGfCfaccuaaua | 3966 | VPusGfsuauUfaGfGfugcaUfuGfcccags | 4235 | CUCUGGGCAAUGCACCUAA | 4504 |
| 1735518 | scsa | | asg | | UACU | |
| AD- | asusuuc(Cda)CfuCfUfGfccuaaaga | 3967 | VPusGfsucuUfuAfGfgcagAfgGfgaaaus | 4236 | UAAUUUCCCUCUGCCUAAA | 4505 |
| 1735519 | scsa | | usa | | GACU | |
| AD- | csgscuu(Cda)UfgCfAfAfcguggagc | 3968 | VPusAfsgcuCfcAfCfguugCfaGfaagcgs | 4237 | GCCGCUUCUGCAACGUGGA | 4506 |
| 1735520 | susa | | gsc | | GCUG | |
| AD- | asgsauu(Cda)AfuAfUfUfguauuuc | 3969 | VPusGfsggaAfaUfAfcaauAfuGfaaucus | 4238 | AUAGAUUCAUAUUGUAUUU | 4507 |
| 1735521 | cscsa | | asu | | CCCA | |
| AD- | asgsgac(Ada)AfaUfGfCfauuucuag | 3970 | VPusAfscuaGfaAfAfugcaUfuUfguccus | 4239 | GGAGGACAAAUGCAUUUCU | 4508 |
| 1735522 | susa | | CSC | | AGUG | |
| AD- | ususaga(Cda)GfuGfUfUfgagugag | 3971 | VPusCfsacuCfaCfUfcaacAfcGfucuaas | 4240 | UAUUAGACGUGUUGAGUGA | 4509 |
| 1735523 | usgsa | | usa | | GUGC | |
| AD- | csusgug(Gda)AfcAfUfCfaaaugcug | 3972 | VPusUfscagCfaUfUfugauGfuCfcacags | 4241 | AGCUGUGGACAUCAAAUGC | 4510 |
| 1735524 | sasa | | csu | | UGAA | |
| AD- | asgsgcc(Uda)UfaAfUfAfcugugaga | 3973 | VPusCfsucuCfaCfAfguauUfaAfggccus | 4242 | ACAGGCCUUAAUACUGUGA | 4511 |
| 1735525 | sgsa | | gsu | | GAGG | |
| AD- | asgsaaa(Uda)CfaUfUfGfccuagugu | 3974 | VPusAfsacaCfuAfGfgcaaUfgAfuuucus | 4243 | GCAGAAAUCAUUGCCUAGU | 4512 |
| 1735526 | susa | | gsc | | GUUC | |
| AD- | usgsagu(Gda)CfuGfAfGfuuccuug | 3975 | VPusAfsgcaAfgGfAfacucAfgCfacucas | 4244 | AGUGAGUGCUGAGUUCCUU | 4513 |
| 1735527 | csusa | | csu | | GCUG | |
| AD- | gscscau(Gda)CfaUfGfAfuuaugauc | 3976 | VPusUfsgauCfaUfAfaucaUfgCfauggcs | 4245 | CUGCCAUGCAUGAUUAUGA | 4514 |
| 1735528 | sasa | | asg | | UCAC | |
| AD- | usgscug(Cda)UfgGfUfUfugcuacca | 3977 | VPusCfsuggUfaGfCfaaacCfaGfcagcas | 4246 | GAUGCUGCUGGUUUGCUAC | 4515 |
| 1735529 | sgsa | | usc | | CAGG | |
| AD- | csasgau(Gda)AfaGfAfGfgaauagcg | 3978 | VPusUfscgcUfaUfUfccucUfuCfaucugs | 4247 | GGCAGAUGAAGAGGAAUAG | 4516 |
| 1735530 | sasa | | CSC | | CGAC | |
| AD- | asuscca(Gda)GfuGfCfAfucaauuuc | 3979 | VPusAfsgaaAfuUfGfaugcAfcCfuggaus | 4248 | AAAUCCAGGUGCAUCAAUU | 4517 |
| 1735531 | susa | | usu | | UCUG | |
| AD- | asasuaa(Gda)CfcAfAfGfcguuaucu | 3980 | VPusAfsagaUfaAfCfgcuuGfgCfuuauus | 4249 | UUAAUAAGCCAAGCGUUAU | 4518 |
| 1735532 | susa | | asa | | CUUG | |
| AD- | gsasgag(Gda)AfuCfCfUfugcucugc | 3981 | VPusAfsgcaGfaGfCfaaggAfuCfcucucs | 4250 | GUGAGAGGAUCCUUGCUCU | 4519 |
| 1735533 | susa | | asc | | GCUG | |
| AD- | asusgau(Gda)AfaGfAfCfggugaaga | 3982 | VPusUfsucuUfcAfCfcgucUfuCfaucaus | 4251 | UGAUGAUGAAGACGGUGAA | 4520 |
| 1735534 | sasa | | csa | | GAAU | |
| AD- | uscsgua(Ada)GfuGfUfAfagaaagaa | 3983 | VPusAfsuucUfuUfCfuuacAfcUfuacgas | 4252 | AUUCGUAAGUGUAAGAAAG | 4521 |
| 1735535 | susa | | asu | | AAUG | |
| AD- | asuscug(Uda)GfuAfAfCfagcugug | 3984 | VPusUfsccaCfaGfCfuguuAfcAfcagaus | 4253 | CAAUCUGUGUAACAGCUGU | 4522 |
| 1735536 | gsasa | | usg | | GGAC | |
| AD- | asusuua(Ada)AfaUfUfCfccacucaa | 3985 | VPusGfsuugAfgUfGfggaaUfuUfuaaau | 4254 | AUAUUUAAAAUUCCCACUC | 4523 |
| 1735537 | scsa | | sasu | | AACA | |
| AD- | gsascca(Gda)UfgUfCfUfacuggcuc | 3986 | VPusAfsgagCfcAfGfuagaCfaCfuggucs | 4255 | UGGACCAGUGUCUACUGGC | 4524 |
| 1735538 | susa | | csa | | UCUC | |
| AD- | csgscuu(Uda)CfgUfUfUfuuuaguc | 3987 | VPusAfsugaCfuAfAfaaaaCfgAfaagcgs | 4256 | AGCGCUUUCGUUUUUUAGU | 4525 |
| 1735539 | asusa | | csu | | CAUU | |
| AD- | uscscgu(Gda)UfuGfAfCfccauguu | 3988 | VPusGfscaaCfaUfGfggucAfaCfacggas | 4257 | CAUCCGUGUUGACCCAUGU | 4526 |
| 1735540 | gscsa | | usg | | UGCA | |
| AD- | asusaca(Gda)AfaGfAfUfgaagaaau | 3989 | VPusCfsauuUfcUfUfcaucUfuCfuguaus | 4258 | AGAUACAGAAGAUGAAGAA | 4527 |
| 1735541 | sgsa | | csu | | AUGG | |
| AD- | uscsucc(Cda)GfaGfCfUfgaaacuua | 3990 | VPusUfsuaaGfuUfUfcagcUfcGfggagas | 4259 | UCUCUCCCGAGCUGAAACU | 4528 |
| 1735542 | sasa | | gsa | | UAAA | |
| AD- | usgscau(Cda)AfaAfCfUfggcagaua | 3991 | VPusAfsuauCfuGfCfcaguUfuGfaugcas | 4260 | UGUGCAUCAAACUGGCAGA | 4529 |
| 1735543 | susa | | csa | | UAUA | |
| AD- | ususgca(Ada)AfaUfAfUfucaggaga | 3992 | VPusGfsucuCfcUfGfaauaUfuUfugcaas | 4261 | AGUUGCAAAAUAUUCAGGA | 4530 |
| 1735544 | scsa | | csu | | GACC | |
| AD- | ascsaau(Cda)GfuAfUfAfcaugccac | 3993 | VPusUfsgugGfcAfUfguauAfcGfauugu | 4262 | UCACAAUCGUAUACAUGCC | 4531 |
| 1735545 | sasa | | sgsa | | ACAG | |
| AD- | asasuag(Ada)AfaAfGfAfugagcaac | 3994 | VPusAfsguuGfcUfCfaucuUfuUfcuauus | 4263 | UUAAUAGAAAAGAUGAGCA | 4532 |
| 1735546 | susa | | asa | | ACUG | |
| AD- | usasucu(Uda)AfuGfUfUfccauggc | 3995 | VPusCfsagcCfaUfGfgaacAfuAfagauas | 4264 | CUUAUCUUAUGUUCCAUGG | 4533 |
| 1735547 | usgsa | | asg | | CUGA | |
| AD- | csusacg(Uda)GfaAfGfAfgcugcgu | 3996 | VPusUfscacGfcAfGfcucuUfcAfcguags | 4265 | GGCUACGUGAAGAGCUGCG | 4534 |
| 1735548 | gsasa | | CSC | | UGAG | |
| AD- | gsasauu(Gda)AfuGfGfCfucuauacg | 3997 | VPusAfscguAfuAfGfagccAfuCfaauucs | 4266 | UAGAAUUGAUGGCUCUAUA | 4535 |
| 1735549 | susa | | usa | | CGUG | |
| AD- | ususcag(Gda)UfaAfUfUfgaagaggc | 3998 | VPusUfsgccUfcUfUfcaauUfaCfcugaas | 4267 | GAUUCAGGUAAUUGAAGAG | 4536 |
| 1735550 | sasa | | usc | | GCAG | |
| AD- | asasgga(Cda)UfgAfGfUfgaccuuug | 3999 | VPusAfscaaAfgGfUfcacuCfaGfuccuus | 4268 | UGAAGGACUGAGUGACCUU | 4537 |
| 1735551 | susa | | csa | | UGUU | |
| AD- | csgsaga(Gda)UfcUfGfAfggaacggc | 4000 | VPusAfsgccGfuUfCfcucaGfaCfucucgs | 4269 | CCCGAGAGUCUGAGGAACG | 4538 |
| 1735552 | susa | | gsg | | GCUA | |
| AD- | asusucu(Uda)UfgGfGfAfuugggac | 4001 | VPusAfsaguCfcCfAfauccCfaAfagaaus | 4270 | UGAUUCUUUGGGAUUGGGA | 4539 |
| 1735553 | ususa | | csa | | CUUA | |
| AD- | usascca(Gda)GfuCfAfGfugguuaga | 4002 | VPusUfsucuAfaCfCfacugAfcCfugguas | 4271 | GCUACCAGGUCAGUGGUUA | 4540 |
| 1735554 | sasa | | gsc | | GAAG | |
| AD- | asusuca(Gda)GfuUfUfGfccauggac | 4003 | VPusUfsgucCfaUfGfgcaaAfcCfugaaus | 4272 | GUAUUCAGGUUUGCCAUGG | 4541 |
| 1735555 | sasa | | asc | | ACAU | |
| AD- | gscsagc(Uda)AfaAfCfUfgguccugg | 4004 | VPusUfsccaGfgAfCfcaguUfuAfgcugcs | 4273 | CGGCAGCUAAACUGGUCCU | 4542 |
| 1735556 | sasa | | csg | | GGAG | |
| AD- | asgsuaa(Uda)GfgCfAfUfagaaugga | 4005 | VPusCfsuccAfuUfCfuaugCfcAfuuacus | 4274 | AUAGUAAUGGCAUAGAAUG | 4543 |
| 1735557 | sgsa | | asu | | GAGU | |
| AD- | asasuug(Cda)AfaGfAfAfaauggacc | 4006 | VPusUfsgguCfcAfUfuuucUfuGfcaauus | 4275 | AUAAUUGCAAGAAAAUGGA | 4544 |
| 1735558 | sasa | | asu | | CCAU | |
| AD- | csgsgca(Cda)UfgCfGfUfucuggugc | 4007 | VPusAfsgcaCfcAfGfaacgCfaGfugccgs | 4276 | UCCGGCACUGCGUUCUGGU | 4545 |
| 1735559 | susa | | gsa | | GCUG | |
| AD- | asgsuca(Uda)AfuUfGfAfagaagccc | 4008 | VPusUfsgggCfuUfCfuucaAfuAfugacus | 4277 | AAAGUCAUAUUGAAGAAGC | 4546 |
| 1735560 | sasa | | usu | | CCAG | |
| AD- | uscsauc(Ada)UfgCfUfGfcgucagcu | 4009 | VPusAfsagcUfgAfCfgcagCfaUfgaugas | 4278 | AAUCAUCAUGCUGCGUCAG | 4547 |
| 1735561 | susa | | usu | | CUUG | |
| AD- | asasaug(Ada)UfcGfCfCfucuuggua | 4010 | VPusAfsuacCfaAfGfaggcGfaUfcauuus | 4279 | GAAAAUGAUCGCCUCUUGG | 4548 |
| 1735562 | susa | | usc | | UAUG | |
| AD- | asusuug(Ada)AfgAfUfAfuaaagag | 4011 | VPusUfsgcuCfuUfUfauauCfuUfcaaaus | 4280 | ACAUUUGAAGAUAUAAAGA | 4549 |
| 1735563 | csasa | | gsu | | GCAG | |
| AD- | gsgsucu(Cda)CfuUfCfAfccagccuc | 4012 | VPusCfsgagGfcUfGfgugaAfgGfagaccs | 4281 | UGGGUCUCCUUCACCAGCC | 4550 |
| 1735564 | sgsa | | csa | | UCGG | |
| AD- | gsasaac(Ada)GfaGfAfAfgaaagaca | 4013 | VPusCfsuguCfuUfUfcuucUfcUfguuucs | 4282 | AGGAAACAGAGAAGAAAGA | 4551 |
| 1735565 | sgsa | | csu | | CAGC | |
| AD- | ususcua(Gda)UfgGfCfUfacaaaugc | 4014 | VPusGfsgcaUfuUfGfuagcCfaCfuagaas | 4283 | AUUUCUAGUGGCUACAAAU | 4552 |
| 1735566 | scsa | | asu | | GCCC | |
| AD- | gsgsaga(Gda)AfaAfUfCfaggaagga | 4015 | VPusAfsuccUfuCfCfugauUfuCfucuccs | 4284 | UGGGAGAGAAAUCAGGAAG | 4553 |
| 1735567 | susa | | csa | | GAUU | |
| AD- | asusaua(Gda)UfgAfGfCfccuaaagg | 4016 | VPusUfsccuUfuAfGfggcuCfaCfuauaus | 4285 | AAAUAUAGUGAGCCCUAAA | 4554 |
| 1735568 | sasa | | usu | | GGAC | |
| AD- | gscsugc(Cda)UfuUfGfUfccucgccc | 4017 | VPusAfsgggCfgAfGfgacaAfaGfgcagcs | 4286 | UGGCUGCCUUUGUCCUCGC | 4555 |
| 1735569 | susa | | csa | | CCUG | |
| AD- | gsgscaa(Ada)AfgAfCfCfucaccaag | 4018 | VPusUfscuuGfgUfGfagguCfuUfuugcc | 4287 | AUGGCAAAAGACCUCACCA | 4556 |
| 1735570 | sasa | | sasu | | AGAA | |
| AD- | ususcag(Cda)AfuCfGfCfcugugccu | 4019 | VPusAfsaggCfaCfAfggcgAfuGfcugaas | 4288 | UCUUCAGCAUCGCCUGUGC | 4557 |
| 1735571 | susa | | gsa | | CUUC | |
| AD- | gscsuaa(Ada)UfgAfGfGfcucgcaau | 4020 | VPusUfsauuGfcGfAfgccuCfaUfuuagcs | 4289 | GUGCUAAAUGAGGCUCGCA | 4558 |
| 1735572 | sasa | | asc | | AUAU | |
| AD- | csasgcu(Uda)CfgUfCfUfggugggu | 4021 | VPusAfsgacCfcAfCfcagaCfgAfagcugs | 4290 | GCCAGCUUCGUCUGGUGGG | 4559 |
| 1735573 | csusa | | gsc | | UCUC | |
| AD- | asusaug(Gda)UfaUfUfGfgcaugcag | 4022 | VPusAfscugCfaUfGfccaaUfaCfcauaus | 4291 | GAAUAUGGUAUUGGCAUGC | 4560 |
| 1735574 | susa | | usc | | AGUU | |
| AD- | asasacu(Gda)CfaGfGfUfggagaauu | 4023 | VPusGfsaauUfcUfCfcaccUfgCfaguuus | 4292 | AUAAACUGCAGGUGGAGAA | 4561 |
| 1735575 | scsa | | asu | | UUCC | |
| AD- | usasugg(Cda)UfgCfCfUfgucuucaa | 4024 | VPusUfsuugAfaGfAfcaggCfaGfccauas | 4293 | GUUAUGGCUGCCUGUCUUC | 4562 |
| 1735576 | sasa | | asc | | AAAC | |
| AD- | csusguu(Cda)UfgAfAfAfuagagga | 4025 | VPusGfsgucCfuCfUfauuuCfaGfaacags | 4294 | CCCUGUUCUGAAAUAGAGG | 4563 |
| 1735577 | cscsa | | gsg | | ACCC | |
| AD- | uscsuau(Uda)UfuUfAfAfagccgcu | 4026 | VPusCfscagCfgGfCfuuuaAfaAfauagas | 4295 | UUUCUAUUUUUAAAGCCGC | 4564 |
| 1735578 | gsgsa | | asa | | UGGU | |
| AD- | gscsuga(Gda)GfaGfAfAfgcucagga | 4027 | VPusUfsuccUfgAfGfcuucUfcCfucagcs | 4296 | CAGCUGAGGAGAAGCUCAG | 4565 |
| 1735579 | sasa | | usg | | GAAC | |
| AD- | csasagc(Cda)UfuCfCfAfccacaagu | 4028 | VPusGfsacuUfgUfGfguggAfaGfgcuug | 4297 | UCCAAGCCUUCCACCACAA | 4566 |
| 1735580 | scsa | | sgsa | | GUCA | |
| AD- | ususcag(Uda)UfaCfCfAfaaguggcc | 4029 | VPusUfsggcCfaCfUfuuggUfaAfcugaas | 4298 | AUUUCAGUUACCAAAGUGG | 4567 |
| 1735581 | sasa | | asu | | CCAG | |
| AD- | csasccu(Gda)CfuUfCfCfucacccgg | 4030 | VPusUfsccgGfgUfGfaggaAfgCfaggugs | 4299 | CUCACCUGCUUCCUCACCC | 4568 |
| 1735582 | sasa | | asg | | GGAC | |
| AD- | ususuua(Uda)CfaCfCfCfacauagug | 4031 | VPusCfscacUfaUfGfugggUfgAfuaaaas | 4300 | UCUUUUAUCACCCACAUAG | 4569 |
| 1735583 | sgsa | | gsa | | UGGG | |
| AD- | asgsgag(Ada)CfcGfUfCfguugccuu | 4032 | VPusCfsaagGfcAfAfcgacGfgUfcuccus | 4301 | UCAGGAGACCGUCGUUGCC | 4570 |
| 1735584 | sgsa | | gsa | | UUGU | |
| AD- | asusggc(Cda)CfaGfCfAfaaaguucg | 4033 | VPusUfscgaAfcUfUfuugcUfgGfgccaus | 4302 | AAAUGGCCCAGCAAAAGUU | 4571 |
| 1735585 | sasa | | usu | | CGAG | |
| AD- | asgsacg(Cda)CfaAfAfGfccaugcgg | 4034 | VPusAfsccgCfaUfGfgcuuUfgGfcgucus | 4303 | CGAGACGCCAAAGCCAUGC | 4572 |
| 1735586 | susa | | csg | | GGUC | |
| AD- | csasgcu(Ada)AfuGfCfAfccaccuca | 4035 | VPusGfsugaGfgUfGfgugcAfuUfagcug | 4304 | GUCAGCUAAUGCACCACCU | 4573 |
| 1735587 | scsa | | sasc | | CACU | |
| AD- | gsgsaaa(Ada)GfcAfGfAfcggcgaau | 4036 | VPusUfsauuCfgCfCfgucuGfcUfuuuccs | 4305 | AAGGAAAAGCAGACGGCGA | 4574 |
| 1735588 | sasa | | usu | | AUAU | |
| AD- | gscsuuu(Gda)CfuGfGfGfuagugag | 4037 | VPusAfsgcuCfaCfUfacccAfgCfaaagcs | 4306 | CUGCUUUGCUGGGUAGUGA | 4575 |
| 1735589 | csusa | | asg | | GCUC | |

TABLE 14
Unmodified Sense and Antisense Strand Sequences of PDE3B
dsRNA Agents Comprising a GalNAc Derivative Targeting Ligand

| | | SEQ | | | SEQ | |
|---|---|---|---|---|---|---|
| Duplex | Sense Sequence | ID | Range in | Antisense Sequence | ID | Range in |
| Name | 5′ to 3′ | NO: | NM_000922.4 | 5′ to 3′ | NO: | NM_000922.4 |
| AD-1735591 | UUCCUUCUUCAUCUUGAUCA | 3231 | 2880-2900 | UUGAUCAAGAUGAAGAAGGAAG | 3500 | 2878-2900 |
| | A | | | U | | |
| AD-1735592 | CACUCUAAAACUGAUGUUCA | 3232 | 4621-4641 | UUGAACAUCAGUUUUAGAGUGA | 3501 | 4619-4641 |
| | A | | | A | | |
| AD-1735593 | UCUUCUGUAUCACUGACUCA | 3233 | 1752-1772 | UUGAGUCAGUGAUACAGAAGAU | 3502 | 1750-1772 |
| | A | | | G | | |
| AD-1735594 | CAGAUCUUCUGACUGAUCCA | 3234 | 1363-1383 | UUGGAUCAGUCAGAAGAUCUGA | 3503 | 1361-1383 |
| | A | | | C | | |
| AD-1735595 | UGACACAUUUUUCUCCAUAA | 3235 | 5958-5978 | UUUAUGGAGAAAAAUGUGUCAG | 3504 | 5956-5978 |
| | A | | | U | | |
| AD-1735596 | GGAAACUCAUUUUUACAUAA | 3236 | 5617-5637 | UUUAUGUAAAAAUGAGUUUCCA | 3505 | 5615-5637 |
| | A | | | G | | |
| AD-1735597 | AUCAGCAACUUAGAAAUUCU | 3237 | 1966-1986 | UAGAAUUUCUAAGUUGCUGAUA | 3506 | 1964-1986 |
| | A | | | A | | |
| AD-1735598 | CUUAUAUUUCUCUAGAGUAC | 3238 | 5351-5371 | UGUACUCUAGAGAAAUAUAAGG | 3507 | 5349-5371 |
| | A | | | A | | |
| AD-1735599 | AACUCCAAGAAUCUUUUAUC | 3239 | 3229-3249 | UGAUAAAAGAUUCUUGGAGUUU | 3508 | 3227-3249 |
| | A | | | U | | |
| AD-1735600 | CUUCAAACAUUCCUGCAUUA | 3240 | 2680-2700 | UUAAUGCAGGAAUGUUUGAAGA | 3509 | 2678-2700 |
| | A | | | C | | |
| AD-1735601 | UCGUUCUUCUCCUCAACUAG | 3241 | 3206-3226 | UCUAGUUGAGGAGAAGAACGAU | 3510 | 3204-3226 |
| | A | | | C | | |
| AD-1735602 | AAAGAUUAAUCCUCUCACAC | 3242 | 1460-1480 | UGUGUGAGAGGAUUAAUCUUUG | 3511 | 1458-1480 |
| | A | | | G | | |
| AD-1735603 | ACUAACCUCAUCAAAGAUAC | 3243 | 4501-4521 | UGUAUCUUUGAUGAGGUUAGUU | 3512 | 4499-4521 |
| | A | | | U | | |
| AD-1735604 | CACCACUUAUUACUAAUAAA | 3244 | 5283-5303 | UUUUAUUAGUAAUAAGUGGUGG | 3513 | 5281-5303 |
| | A | | | G | | |
| AD-1735605 | UGGUACUAGAAAUAUUCUUU | 3245 | 5915-5935 | UAAAGAAUAUUUCUAGUACCAG | 3514 | 5913-5935 |
| | A | | | C | | |
| AD-1735606 | GCUGUCAUUUGUUCACUCUA | 3246 | 4608-4628 | UUAGAGUGAACAAAUGACAGCU | 3515 | 4606-4628 |
| | A | | | A | | |
| AD-1735607 | ACUCAACAAUUUAUGAACUA | 3247 | 2421-2441 | UUAGUUCAUAAAUUGUUGAGUG | 3516 | 2419-2441 |
| | A | | | G | | |
| AD-1735608 | AGUAUGACUCAUUAAUAGAA | 3248 | 2269-2289 | UUUCUAUUAAUGAGUCAUACUC | 3517 | 2267-2289 |
| | A | | | U | | |
| AD-1735609 | CUGCUGAUUUUCUUAAUAAG | 3249 | 1891-1911 | UCUUAUUAAGAAAAUCAGCAGU | 3518 | 1889-1911 |
| | A | | | A | | |
| AD-1735610 | UAUCUUUAGAUCCACAUCUU | 3250 | 4587-4607 | UAAGAUGUGGAUCUAAAGAUAA | 3519 | 4585-4607 |
| | A | | | C | | |
| AD-1735611 | CGAGACAUUCCUUAUCACAA | 3251 | 2466-2486 | UUUGUGAUAAGGAAUGUCUCGA | 3520 | 2464-2486 |
| | A | | | U | | |
| AD-1735612 | UACUGAAUCAGAUUAAUUUC | 3252 | 5850-5870 | UGAAAUUAAUCUGAUUCAGUAA | 3521 | 5848-5870 |
| | A | | | C | | |
| AD-1735613 | CUUACUUAAAUCCUUCACUG | 3253 | 3855-3875 | UCAGUGAAGGAUUUAAGUAAGG | 3522 | 3853-3875 |
| | A | | | G | | |
| AD-1735614 | UCGAAAGAAUCAUUCAAACU | 3254 | 2100-2120 | UAGUUUGAAUGAUUCUUUCGAG | 3523 | 2098-2120 |
| | A | | | A | | |
| AD-1735615 | AAAUGACCUAUCACUACUUA | 3255 | 4682-4702 | UUAAGUAGUGAUAGGUCAUUUC | 3524 | 4680-4702 |
| | A | | | U | | |
| AD-1735616 | CAAGUCAUUUCCUCUCUACG | 3256 | 1395-1415 | UCGUAGAGAGGAAAUGACUUGU | 3525 | 1393-1415 |
| | A | | | G | | |
| AD-1735617 | AUGCAGUUUCUUACUUAUCU | 3257 | 5079-5099 | UAGAUAAGUAAGAAACUGCAUG | 3526 | 5077-5099 |
| | A | | | C | | |
| AD-1735618 | UCAAUGCAAUACACUGUUCA | 3258 | 4124-4144 | UUGAACAGUGUAUUGCAUUGAG | 3527 | 4122-4144 |
| | A | | | C | | |
| AD-1735619 | UCUAUGUACCUACUGACACA | 3259 | 5945-5965 | UUGUGUCAGUAGGUACAUAGAU | 3528 | 5943-5965 |
| | A | | | A | | |
| AD-1735620 | UGAAUUCCAGUCUUAUCUUA | 3260 | 4312-4332 | UUAAGAUAAGACUGGAAUUCAA | 3529 | 4310-4332 |
| | A | | | A | | |
| AD-1735621 | UUGCUGUAAUACCAAAACUA | 3261 | 4485-4505 | UUAGUUUUGGUAUUACAGCAAA | 3530 | 4483-4505 |
| | A | | | U | | |
| AD-1735622 | GGAUCACUUCUUUCAAAUCA | 3262 | 1061-1081 | UUGAUUUGAAAGAAGUGAUCCA | 3531 | 1059-1081 |
| | A | | | A | | |
| AD-1735623 | GAACUAGAAAACUAUUCUUA | 3263 | 3795-3815 | UUAAGAAUAGUUUUCUAGUUCC | 3532 | 3793-3815 |
| | A | | | U | | |
| AD-1735624 | ACAUUUCCAUACUGUCUGUU | 3264 | 5484-5504 | UAACAGACAGUAUGGAAAUGUG | 3533 | 5482-5504 |
| | A | | | A | | |
| AD-1735625 | UGAUCCUUCACAGUGUCAAU | 3265 | 5158-5178 | UAUUGACACUGUGAAGGAUCAA | 3534 | 5156-5178 |
| | A | | | A | | |
| AD-1735626 | GAUUUCUGAGAACAAGUAAA | 3266 | 4729-4749 | UUUUACUUGUUCUCAGAAAUCA | 3535 | 4727-4749 |
| | A | | | G | | |
| AD-1735627 | CCUACUUGUGAAAUACAUUU | 3267 | 4906-4926 | UAAAUGUAUUUCACAAGUAGGG | 3536 | 4904-4926 |
| | A | | | U | | |
| AD-1735628 | CAACUGGAAUUUUCCAAUUU | 3268 | 2297-2317 | UAAAUUGGAAAAUUCCAGUUGC | 3537 | 2295-2317 |
| | A | | | U | | |
| AD-1735629 | UCUAAUCCUGAUGAGAGUUA | 3269 | 2649-2669 | UUAACUCUCAUCAGGAUUAGAG | 3538 | 2647-2669 |
| | A | | | C | | |
| AD-1735630 | AGCCUCAUUAUCAAAAUUCU | 3270 | 1288-1308 | UAGAAUUUUGAUAAUGAGGCUU | 3539 | 1286-1308 |
| | A | | | A | | |
| AD-1735631 | AAUUUCUGAUGCUUUUUACU | 3271 | 4220-4240 | UAGUAAAAAGCAUCAGAAAUUG | 3540 | 4218-4240 |
| | A | | | A | | |
| AD-1735632 | UACCAUUAGAUGAAAUCUUA | 3272 | 5788-5808 | UUAAGAUUUCAUCUAAUGGUAC | 3541 | 5786-5808 |
| | A | | | A | | |
| AD-1735633 | CUGUGAAUUCUUCCAACCAU | 3273 | 1816-1836 | UAUGGUUGGAAGAAUUCACAGG | 3542 | 1814-1836 |
| | A | | | A | | |
| AD-1735634 | CUAAUACUCCAGAUUUUUAU | 3274 | 1948-1968 | UAUAAAAAUCUGGAGUAUUAGG | 3543 | 1946-1968 |
| | A | | | U | | |
| AD-1735635 | GGACACAUACAUUGAAUAAA | 3275 | 4550-4570 | UUUUAUUCAAUGUAUGUGUCCU | 3544 | 4548-4570 |
| | A | | | U | | |
| AD-1735636 | UUUGUCAUAAUGCUGCUUUG | 3276 | 3887-3907 | UCAAAGCAGCAUUAUGACAAAG | 3545 | 3885-3907 |
| | A | | | U | | |
| AD-1735637 | UUCAUCUUUCUGUUAAUUUC | 3277 | 4637-4657 | UGAAAUUAACAGAAAGAUGAAC | 3546 | 4635-4657 |
| | A | | | A | | |
| AD-1735638 | CAGUGAAACCAUUAAUUUUC | 3278 | 5028-5048 | UGAAAAUUAAUGGUUUCACUGU | 3547 | 5026-5048 |
| | A | | | G | | |
| AD-1735639 | UAACUGCAAGACUGAUUUCU | 3279 | 4716-4736 | UAGAAAUCAGUCUUGCAGUUAG | 3548 | 4714-4736 |
| | A | | | G | | |
| AD-1735640 | AGAAUUCCUCCUUACCUCAA | 3280 | 3547-3567 | UUUGAGGUAAGGAGGAAUUCUC | 3549 | 3545-3567 |
| | A | | | C | | |
| AD-1735641 | UAUUACAGAUCUUACUUUGU | 3281 | 5830-5850 | UACAAAGUAAGAUCUGUAAUAU | 3550 | 5828-5850 |
| | A | | | G | | |
| AD-1735642 | UUAUCUGAAAGAUAUCAAUU | 3282 | 5111-5131 | UAAUUGAUAUCUUUCAGAUAAG | 3551 | 5109-5131 |
| | A | | | C | | |
| AD-1735643 | AGAUCUGUUCUGGAAAAUCA | 3283 | 2811-2831 | UUGAUUUUCCAGAACAGAUCUG | 3552 | 2809-2831 |
| | A | | | U | | |
| AD-1735644 | CCCAGAAUACAACUUCCUUC | 3284 | 2867-2887 | UGAAGGAAGUUGUAUUCUGGGC | 3553 | 2865-2887 |
| | A | | | G | | |
| AD-1735645 | GUUGAUCUUUCAGUGCUAAA | 3285 | 1323-1343 | UUUUAGCACUGAAAGAUCAACU | 3554 | 1321-1343 |
| | A | | | C | | |
| AD-1735646 | CUAAGCUUUAUUUAUUAGAC | 3286 | 5191-5211 | UGUCUAAUAAAUAAAGCUUAGU | 3555 | 5189-5211 |
| | A | | | G | | |
| AD-1735647 | AGCUUGGAAUCUAUAUCUUU | 3287 | 2843-2863 | UAAAGAUAUAGAUUCCAAGCUG | 3556 | 2841-2863 |
| | A | | | A | | |
| AD-1735648 | UUAUUAUUUCUGAAGCCUAA | 3288 | 4699-4719 | UUUAGGCUUCAGAAAUAAUAAG | 3557 | 4697-4719 |
| | A | | | U | | |
| AD-1735649 | CCUUUGUUGUAUUUAACAAA | 3289 | 4187-4207 | UUUUGUUAAAUACAACAAAGGU | 3558 | 4185-4207 |
| | A | | | C | | |
| AD-1735650 | GCUCUCUAACUAAUCGAUCA | 3290 | 1852-1872 | UUGAUCGAUUAGUUAGAGAGCC | 3559 | 1850-1872 |
| | A | | | A | | |
| AD-1735651 | AAAGACUACAUGACAGAAAU | 3291 | 4666-4686 | UAUUUCUGUCAUGUAGUCUUUA | 3560 | 4664-4686 |
| | A | | | G | | |
| AD-1735652 | CAGGAUUCGUAUUUUUAAAG | 3292 | 4418-4438 | UCUUUAAAAAUACGAAUCCUGA | 3561 | 4416-4438 |
| | A | | | G | | |
| AD-1735653 | CCUUGCUAAGUAAUUGACAU | 3293 | 5659-5679 | UAUGUCAAUUACUUAGCAAGGG | 3562 | 5657-5679 |
| | A | | | U | | |
| AD-1735654 | CCUAUGCACUUUCACAGGAA | 3294 | 3778-3798 | UUUCCUGUGAAAGUGCAUAGGA | 3563 | 3776-3798 |
| | A | | | G | | |
| AD-1735655 | AUGCUGAAAUAUGUUUCAAC | 3295 | 2019-2039 | UGUUGAAACAUAUUUCAGCAUU | 3564 | 2017-2039 |
| | A | | | U | | |
| AD-1735656 | CCUUUUAAUUCAAAUCUACU | 3296 | 1710-1730 | UAGUAGAUUUGAAUUAAAAGGC | 3565 | 1708-1730 |
| | A | | | C | | |
| AD-1735657 | ACUGGACUUAUUGGAUUUAA | 3297 | 5577-5597 | UUUAAAUCCAAUAAGUCCAGUA | 3566 | 5575-5597 |
| | A | | | U | | |
| AD-1735658 | CAUCUGAAUCAGAUGGUACA | 3298 | 2038-2058 | UUGUACCAUCUGAUUCAGAUGU | 3567 | 2036-2058 |
| | A | | | U | | |
| AD-1735659 | AAUUCUGAUAGCAAUCUGUG | 3299 | 1980-2000 | UCACAGAUUGCUAUCAGAAUUU | 3568 | 1978-2000 |
| | A | | | C | | |
| AD-1735660 | UCAAGUCAAGGAUGCUAUCU | 3300 | 1683-1703 | UAGAUAGCAUCCUUGACUUGAA | 3569 | 1681-1703 |
| | A | | | A | | |
| AD-1735661 | UCACUGAAAACCACAAGAUA | 3301 | 3466-3486 | UUAUCUUGUGGUUUUCAGUGAG | 3570 | 3464-3486 |
| | A | | | G | | |
| AD-1735662 | ACCUGUUGAACAGUCUUCAA | 3302 | 1616-1636 | UUUGAAGACUGUUCAACAGGUA | 3571 | 1614-1636 |
| | A | | | G | | |
| AD-1735663 | UCAAGUGACAUAUUUCAGUU | 3303 | 3957-3977 | UAACUGAAAUAUGUCACUUGAG | 3572 | 3955-3977 |
| | A | | | U | | |
| AD-1735664 | UCAGGAACUUCAGGAUUGCU | 3304 | 1596-1616 | UAGCAAUCCUGAAGUUCCUGAG | 3573 | 1594-1616 |
| | A | | | C | | |
| AD-1735665 | ACGGAGUAUUAGUAGCUUAA | 3305 | 1412-1432 | UUUAAGCUACUAAUACUCCGUA | 3574 | 1410-1432 |
| | A | | | G | | |
| AD-1735666 | AUUGUCAAUGAAUUUUAUGA | 3306 | 3135-3155 | UUCAUAAAAUUCAUUGACAAUG | 3575 | 3133-3155 |
| | A | | | C | | |
| AD-1735667 | GAACAGCAAACAAAUAUUGA | 3307 | 2214-2234 | UUCAAUAUUUGUUUGCUGUUCA | 3576 | 2212-2234 |
| | A | | | C | | |
| AD-1735668 | UGAACUAUUUUCGUGCAUUA | 3308 | 2434-2454 | UUAAUGCACGAAAAUAGUUCAU | 3577 | 2432-2454 |
| | A | | | A | | |
| AD-1735669 | ACUGGAAUACUUAUUUUUCA | 3309 | 4751-4771 | UUGAAAAAUAAGUAUUCCAGUU | 3578 | 4749-4771 |
| | A | | | C | | |
| AD-1735670 | UUAUCUUGCAGAGAUCUCUG | 3310 | 1918-1938 | UCAGAGAUCUCUGCAAGAUAAC | 3579 | 1916-1938 |
| | A | | | G | | |
| AD-1735671 | GAAGUGUUAGAAUUUUUGAU | 3311 | 5142-5162 | UAUCAAAAAUUCUAACACUUCC | 3580 | 5140-5162 |
| | A | | | A | | |
| AD-1735672 | UCAAUUUAAGAUCUCUGGAA | 3312 | 5125-5145 | UUUCCAGAGAUCUUAAAUUGAU | 3581 | 5123-5145 |
| | A | | | A | | |
| AD-1735673 | UGGACCAUAUUUACAAUGUU | 3313 | 4856-4876 | UAACAUUGUAAAUAUGGUCCAU | 3582 | 4854-4876 |
| | A | | | U | | |
| AD-1735674 | UAGUCAUUGAAGCAAUCCUU | 3314 | 2926-2946 | UAAGGAUUGCUUCAAUGACUAA | 3583 | 2924-2946 |
| | A | | | A | | |
| AD-1735675 | AGCUGUUUAGGUUUAACAAU | 3315 | 4353-4373 | UAUUGUUAAACCUAAACAGCUU | 3584 | 4351-4373 |
| | A | | | U | | |
| AD-1735676 | CAAGGCAAAUGAUGUAAAUA | 3316 | 2993-3013 | UUAUUUACAUCAUUUGCCUUGG | 3585 | 2991-3013 |
| | A | | | C | | |
| AD-1735677 | AUCCAAUUACUACUGGAAAC | 3317 | 5603-5623 | UGUUUCCAGUAGUAAUUGGAUU | 3586 | 5601-5623 |
| | A | | | U | | |
| AD-1735678 | ACUUACAAUAUAUAUAUCCU | 3318 | 5328-5348 | UAGGAUAUAUAUAUUGUAAGUG | 3587 | 5326-5348 |
| | A | | | C | | |
| AD-1735679 | AUGCAGUUUGGUAUCUGACA | 3319 | 2512-2532 | UUGUCAGAUACCAAACUGCAUG | 3588 | 2510-2532 |
| | A | | | U | | |
| AD-1735680 | CCUUAUUUCAAGACACUGGU | 3320 | 2377-2397 | UACCAGUGUCUUGAAAUAAGGU | 3589 | 2375-2397 |
| | A | | | A | | |
| AD-1735681 | GAAGGGAUUUAUUCUUUAGU | 3321 | 4373-4393 | UACUAAAGAAUAAAUCCCUUCA | 3590 | 4371-4393 |
| | A | | | U | | |
| AD-1735682 | AUCCUUGCUACGGAUCUUAA | 3322 | 2940-2960 | UUUAAGAUCCGUAGCAAGGAUU | 3591 | 2938-2960 |
| | A | | | G | | |
| AD-1735683 | ACUGACUAUCCCGAAGCAAA | 3323 | 1727-1747 | UUUUGCUUCGGGAUAGUCAGUA | 3592 | 1725-1747 |
| | A | | | G | | |
| AD-1735684 | UGUAUUUCUGCUAUUAUUUC | 3324 | 5390-5410 | UGAAAUAAUAGCAGAAAUACAC | 3593 | 5388-5410 |
| | A | | | U | | |
| AD-1735685 | UGUAACAAUAACUAGCCUAA | 3325 | 5680-5700 | UUUAGGCUAGUUAUUGUUACAU | 3594 | 5678-5700 |
| | A | | | A | | |
| AD-1735686 | GUUGUUUUUACUCUAGCUCA | 3326 | 4400-4420 | UUGAGCUAGAGUAAAAACAACA | 3595 | 4398-4420 |
| | A | | | A | | |
| AD-1735687 | CAGGCAGUUUUAUACAAUGA | 3327 | 2790-2810 | UUCAUUGUAUAAAACUGCCUGA | 3596 | 2788-2810 |
| | A | | | G | | |
| AD-1735688 | AGGAUUCUCAGUCAGGUUAU | 3328 | 2352-2372 | UAUAACCUGACUGAGAAUCCUU | 3597 | 2350-2372 |
| | A | | | C | | |
| AD-1735689 | AUUUUCCAUCAUGUUUAAGU | 3329 | 5371-5391 | UACUUAAACAUGAUGGAAAAUG | 3598 | 5369-5391 |
| | A | | | U | | |
| AD-1735690 | UGCCACUUUUGUUACCAUUG | 3330 | 5238-5258 | UCAAUGGUAACAAAAGUGGCAG | 3599 | 5236-5258 |
| | A | | | C | | |
| AD-1735691 | ACUUGCUAGUGUGUGGAUAU | 3331 | 4882-4902 | UAUAUCCACACACUAGCAAGUU | 3600 | 4880-4902 |
| | A | | | U | | |
| AD-1735692 | AAUCUUACUUGAGAAAUUGC | 3332 | 5801-5821 | UGCAAUUUCUCAAGUAAGAUUU | 3601 | 5799-5821 |
| | A | | | C | | |
| AD-1735693 | AGUUCGAGACUUGCAUUUGA | 3333 | 3101-3121 | UUCAAAUGCAAGUCUCGAACUU | 3602 | 3099-3121 |
| | A | | | U | | |
| AD-1735694 | CCAUAGAAUUUCCUGAUACU | 3334 | 1873-1893 | UAGUAUCAGGAAAUUCUAUGGG | 3603 | 1871-1893 |
| | A | | | U | | |
| AD-1735695 | AGGUAAUUCCUUUAGAAUAU | 3335 | 5050-5070 | UAUAUUCUAAAGGAAUUACCUU | 3604 | 5048-5070 |
| | A | | | G | | |
| AD-1735696 | CUUAUGGAAACUCAACAAGA | 3336 | 2118-2138 | UUCUUGUUGAGUUUCCAUAAGU | 3605 | 2116-2138 |
| | A | | | U | | |
| AD-1735697 | AAGCAGCAUUUUAAAUUACG | 3337 | 5554-5574 | UCGUAAUUUAAAAUGCUGCUUU | 3606 | 5552-5574 |
| | A | | | U | | |
| AD-1735698 | UGACUCUCAACUGACCAUUC | 3338 | 3699-3719 | UGAAUGGUCAGUUGAGAGUCAG | 3607 | 3697-3719 |
| | A | | | C | | |
| AD-1735699 | AGGUUUAUCCAUGAAGGACU | 3339 | 4161-4181 | UAGUCCUUCAUGGAUAAACCUA | 3608 | 4159-4181 |
| | A | | | G | | |
| AD-1735700 | ACCAAGAAUUUGGCAUUUCA | 3340 | 1666-1686 | UUGAAAUGCCAAAUUCUUGGUG | 3609 | 1664-1686 |
| | A | | | A | | |
| AD-1735701 | UAUCUAGAAUAUUUGGCUUA | 3341 | 5094-5114 | UUAAGCCAAAUAUUCUAGAUAA | 3610 | 5092-5114 |
| | A | | | G | | |
| AD-1735702 | CUCCUGCAGAAUACAUACAA | 3342 | 5412-5432 | UUUGUAUGUAUUCUGCAGGAGA | 3611 | 5410-5432 |
| | A | | | G | | |
| AD-1735703 | UCACACCAUUUCCUGGAUUU | 3343 | 1474-1494 | UAAAUCCAGGAAAUGGUGUGAG | 3612 | 1472-1494 |
| | A | | | A | | |
| AD-1735704 | AUGUGGAAUUCAAGCGCUUU | 3344 | 2899-2919 | UAAAGCGCUUGAAUUCCACAUG | 3613 | 2897-2919 |
| | A | | | A | | |
| AD-1735705 | AUUGUGUAUUAUCUACUAUG | 3345 | 4240-4260 | UCAUAGUAGAUAAUACACAAUA | 3614 | 4238-4260 |
| | A | | | G | | |
| AD-1735706 | AAUGGAGUAAUGAAAAUGAU | 3346 | 3025-3045 | UAUCAUUUUCAUUACUCCAUUC | 3615 | 3023-3045 |
| | A | | | U | | |
| AD-1735707 | ACUGGUUUAUUGGAAAUAUU | 3347 | 2391-2411 | UAAUAUUUCCAAUAAACCAGUG | 3616 | 2389-2411 |
| | A | | | U | | |
| AD-1735708 | CAAGUGUAUGUGUAUAAAGU | 3348 | 5429-5449 | UACUUUAUACACAUACACUUGU | 3617 | 5427-5449 |
| | A | | | A | | |
| AD-1735709 | UACAAGCAUGCAUAUUGAGA | 3349 | 5457-5477 | UUCUCAAUAUGCAUGCUUGUAC | 3618 | 5455-5477 |
| | A | | | A | | |
| AD-1735710 | UGUGUCUUUUAUGUCUUUGA | 3350 | 4036-4056 | UUCAAAGACAUAAAAGACACAC | 3619 | 4034-4056 |
| | A | | | U | | |
| AD-1735711 | AUUUUCUCGCAGAAUUCAAU | 3351 | 2971-2991 | UAUUGAAUUCUGCGAGAAAAUC | 3620 | 2969-2991 |
| | A | | | A | | |
| AD-1735712 | UAGCGACAGUUUGAGUAAAA | 3352 | 3609-3629 | UUUUUACUCAAACUGUCGCUAU | 3621 | 3607-3629 |
| | A | | | U | | |
| AD-1735713 | CUGGUGUAUGAAUACUUUGU | 3353 | 3872-3892 | UACAAAGUAUUCAUACACCAGU | 3622 | 3870-3892 |
| | A | | | G | | |
| AD-1735714 | GUGAUAAGUGGCUAACAGAA | 3354 | 2182-2202 | UUUCUGUUAGCCACUUAUCACC | 3623 | 2180-2202 |
| | A | | | U | | |
| AD-1735715 | GUAGGCUAAUAUUUUCUAUU | 3355 | 4962-4982 | UAAUAGAAAAUAUUAGCCUACC | 3624 | 4960-4982 |
| | A | | | A | | |
| AD-1735716 | CCUCUUCAUCCUCGACUGUC | 3356 | 1089-1109 | UGACAGUCGAGGAUGAAGAGGC | 3625 | 1087-1109 |
| | A | | | G | | |
| AD-1735717 | AAUUGCCAUAAGCCAUAUUA | 3357 | 5815-5835 | UUAAUAUGGCUUAUGGCAAUUU | 3626 | 5813-5835 |
| | A | | | C | | |
| AD-1735718 | GCGAAUAUUUUGUCAGCUAA | 3358 | 3437-3457 | UUUAGCUGACAAAAUAUUCGCC | 3627 | 3435-3457 |
| | A | | | G | | |
| AD-1735719 | AUCCAGUGUUGCCUUUCUGA | 3359 | 4093-4113 | UUCAGAAAGGCAACACUGGAUU | 3628 | 4091-4113 |
| | A | | | C | | |
| AD-1735720 | AAAGUCUAUGCCUGUCUAAA | 3360 | 4068-4088 | UUUUAGACAGGCAUAGACUUUU | 3629 | 4066-4088 |
| | A | | | C | | |
| AD-1735721 | GGACAUCAGAAGUUUGAAUU | 3361 | 4298-4318 | UAAUUCAAACUUCUGAUGUCCA | 3630 | 4296-4318 |
| | A | | | U | | |
| AD-1735722 | GGCGAAUUGCUUAUAUUUCU | 3362 | 2617-2637 | UAGAAAUAUAAGCAAUUCGCCC | 3631 | 2615-2637 |
| | A | | | A | | |
| AD-1735723 | CACACUAUGUGUAAACCAGU | 3363 | 5261-5281 | UACUGGUUUACACAUAGUGUGU | 3632 | 5259-5281 |
| | A | | | G | | |
| AD-1735724 | CAGGUGAAGAAGAAAACAUU | 3364 | 2077-2097 | UAAUGUUUUCUUCUUCACCUGA | 3633 | 2075-2097 |
| | A | | | U | | |
| AD-1735725 | GUGAGCUCUUAUUUUUCACU | 3365 | 3914-3934 | UAGUGAAAAAUAAGAGCUCACU | 3634 | 3912-3934 |
| | A | | | A | | |
| AD-1735726 | AAAGCUGAUGGGAAUAAACU | 3366 | 3519-3539 | UAGUUUAUUCCCAUCAGCUUUA | 3635 | 3517-3539 |
| | A | | | C | | |
| AD-1735727 | GUGUCGUUAGGAGAAACUGC | 3367 | 1164-1184 | UGCAGUUUCUCCUAACGACACG | 3636 | 1162-1184 |
| | A | | | C | | |
| AD-1735728 | GGGACUUAAAACAAUGGUAU | 3368 | 1267-1287 | UAUACCAUUGUUUUAAGUCCCA | 3637 | 1265-1287 |
| | A | | | A | | |
| AD-1735729 | CUGAUGGUAGAAUUAACCAU | 3369 | 2596-2616 | UAUGGUUAAUUCUACCAUCAGA | 3638 | 2594-2616 |
| | A | | | A | | |
| AD-1735730 | ACUAUGUGUGUUUUAUUUCU | 3370 | 4254-4274 | UAGAAAUAAAACACACAUAGUA | 3639 | 4252-4274 |
| | A | | | G | | |
| AD-1735731 | CUGAUAAUUUAUAUUUGCAC | 3371 | 5310-5330 | UGUGCAAAUAUAAAUUAUCAGU | 3640 | 5308-5330 |
| | A | | | C | | |
| AD-1735732 | GUCCUUAAAUUAUUUAACCC | 3372 | 5641-5661 | UGGGUUAAAUAAUUUAAGGACU | 3641 | 5639-5661 |
| | A | | | A | | |
| AD-1735733 | CGCAAUAUGGUGUCAGAUCU | 3373 | 1350-1370 | UAGAUCUGACACCAUAUUGCGA | 3642 | 1348-1370 |
| | A | | | G | | |
| AD-1735734 | UUCGAAGAGCUGCUCUAAUC | 3374 | 2636-2656 | UGAUUAGAGCAGCUCUUCGAAG | 3643 | 2634-2656 |
| | A | | | A | | |
| AD-1735735 | UUUCUGCUGAGAGUAUUCAG | 3375 | 4269-4289 | UCUGAAUACUCUCAGCAGAAAU | 3644 | 4267-4289 |
| | A | | | A | | |
| AD-1735736 | UUGAACAGGAAGUAUCACUG | 3376 | 2230-2250 | UCAGUGAUACUUCCUGUUCAAU | 3645 | 2228-2250 |
| | A | | | A | | |
| AD-1735737 | UGCCUUGUAUUUCCAGAGAA | 3377 | 1228-1248 | UUUCUCUGGAAAUACAAGGCAA | 3646 | 1226-1248 |
| | A | | | C | | |
| AD-1735738 | GUUACUAUGGCAGUUGCAAA | 3378 | 1189-1209 | UUUUGCAACUGCCAUAGUAACU | 3647 | 1187-1209 |
| | A | | | G | | |
| AD-1735739 | AUGGCUGAAUUUUAAAGCUG | 3379 | 4338-4358 | UCAGCUUUAAAAUUCAGCCAUG | 3648 | 4336-4358 |
| | A | | | G | | |
| AD-1735740 | GUCCAUUCAUGGAUCGUUCU | 3380 | 3193-3213 | UAGAACGAUCCAUGAAUGGACU | 3649 | 3191-3213 |
| | A | | | G | | |
| AD-1735741 | UGUGUAACUCCUAUGAUGCU | 3381 | 3268-3288 | UAGCAUCAUAGGAGUUACACAG | 3650 | 3266-3288 |
| | A | | | G | | |
| AD-1735742 | UGGCAGUUUCCCACUCCUAU | 3382 | 3763-3783 | UAUAGGAGUGGGAAACUGCCAG | 3651 | 3761-3783 |
| | A | | | C | | |
| AD-1735743 | CCUCAAGCAGAUGAGAUUCA | 3383 | 3561-3581 | UUGAAUCUCAUCUGCUUGAGGU | 3652 | 3559-3581 |
| | A | | | A | | |
| AD-1735744 | AUGUUGUUUUCUCAAAAGCA | 3384 | 5539-5559 | UUGCUUUUGAGAAAACAACAUA | 3653 | 5537-5559 |
| | A | | | A | | |
| AD-1735745 | GAGGGAUUUCUGCUCAAUGC | 3385 | 4111-4131 | UGCAUUGAGCAGAAAUCCCUCA | 3654 | 4109-4131 |
| | A | | | G | | |
| AD-1735746 | GCCUUCUUCUUCCUCACCUG | 3386 | 654-674 | UCAGGUGAGGAAGAAGAAGGCA | 3655 | 652-674 |
| | A | | | C | | |
| AD-1735747 | UUGAGAUUGAAUCACAUUUC | 3387 | 5471-5491 | UGAAAUGUGAUUCAAUCUCAAU | 3656 | 5469-5491 |
| | A | | | A | | |
| AD-1735748 | CUGUUCAGUGCUAUUCUCCC | 3388 | 4137-4157 | UGGGAGAAUAGCACUGAACAGU | 3657 | 4135-4157 |
| | A | | | G | | |
| AD-1735749 | CCUGGCUUACAGCAGAUCCA | 3389 | 2544-2564 | UUGGAUCUGCUGUAAGCCAGGA | 3658 | 2542-2564 |
| | A | | | A | | |
| AD-1735750 | GACAGCAGAAAAUUAUUUCA | 3390 | 2157-2177 | UUGAAAUAAUUUUCUGCUGUCU | 3659 | 2155-2177 |
| | A | | | U | | |
| AD-1735751 | UUGGGUUUCUUUAGUUUAUG | 3391 | 5522-5542 | UCAUAAACUAAAGAAACCCAAU | 3660 | 5520-5542 |
| | A | | | A | | |
| AD-1735752 | CACCAUGUAGGUCUCAGAAG | 3392 | 1770-1790 | UCUUCUGAGACCUACAUGGUGA | 3661 | 1768-1790 |
| | A | | | G | | |
| AD-1735753 | GUUGUGGAACAGGAAAUGAA | 3393 | 2569-2589 | UUUCAUUUCCUGUUCCACAACC | 3662 | 2567-2589 |
| | A | | | A | | |
| AD-1735754 | GGAGAACAUAUCAUAUUUUG | 3394 | 4988-5008 | UCAAAAUAUGAUAUGUUCUCCA | 3663 | 4986-5008 |
| | A | | | A | | |
| AD-1735755 | CAGCCAAAAUGAUGGCAAAA | 3395 | 4940-4960 | UUUUUGCCAUCAUUUUGGCUGC | 3664 | 4938-4960 |
| | A | | | U | | |
| AD-1735756 | CCAAUUUUUGAACUUGUAGA | 3396 | 2310-2330 | UUCUACAAGUUCAAAAAUUGGA | 3665 | 2308-2330 |
| | A | | | A | | |
| AD-1735757 | AUAGGAAUAGUUUGCCAACU | 3397 | 1558-1578 | UAGUUGGCAAACUAUUCCUAUU | 3666 | 1556-1578 |
| | A | | | U | | |
| AD-1735758 | UUUGAAUGUUUUGGAGAAAA | 3398 | 4051-4071 | UUUUUCUCCAAAACAUUCAAAG | 3667 | 4049-4071 |
| | A | | | A | | |
| AD-1735759 | GUCAAUAUUUAAUGAAUCAC | 3399 | 5172-5192 | UGUGAUUCAUUAAAUAUUGACA | 3668 | 5170-5192 |
| | A | | | C | | |
| AD-1735760 | UGAGAAUAUAUUAAAUGCAC | 3400 | 5764-5784 | UGUGCAUUUAAUAUAUUCUCAC | 3669 | 5762-5784 |
| | A | | | A | | |
| AD-1735761 | UAACAAGGGACUAAAUAGGA | 3401 | 1544-1564 | UUCCUAUUUAGUCCCUUGUUAA | 3670 | 1542-1564 |
| | A | | | G | | |
| AD-1735762 | GGCUUCUUCUUCCACCUCUG | 3402 | 408-428 | UCAGAGGUGGAAGAAGAAGCCG | 3671 | 406-428 |
| | A | | | C | | |
| AD-1735763 | UUGGAACAUGUGGUUAUCUU | 3403 | 4573-4593 | UAAGAUAACCACAUGUUCCAAU | 3672 | 4571-4593 |
| | A | | | U | | |
| AD-1735764 | CAGAGAACAGAUGAUUCUUU | 3404 | 1241-1261 | UAAAGAAUCAUCUGUUCUCUGG | 3673 | 1239-1261 |
| | A | | | A | | |
| AD-1735765 | CUGUUAUUUUAUUGGGUUUU | 3405 | 5499-5519 | UAAAACCCAAUAAAAUAACAGA | 3674 | 5497-5519 |
| | A | | | C | | |
| AD-1735766 | AUGAAGAAGCAAAUCUUGGU | 3406 | 3163-3183 | UACCAAGAUUUGCUUCUUCAUC | 3675 | 3161-3183 |
| | A | | | U | | |
| AD-1735767 | UUUGGAAUCAUGCAAUUUUG | 3407 | 5004-5024 | UCAAAAUUGCAUGAUUCCAAAA | 3676 | 5002-5024 |
| | A | | | U | | |
| AD-1735768 | CAAUGUUUUGUAAACUUGCU | 3408 | 4869-4889 | UAGCAAGUUUACAAAACAUUGU | 3677 | 4867-4889 |
| | A | | | A | | |
| AD-1735769 | GGAUCGUAAUAAUGGCAAAA | 3409 | 1640-1660 | UUUUUGCCAUUAUUACGAUCCC | 3678 | 1638-1660 |
| | A | | | A | | |
| AD-1735770 | GUGCUUUCUCAGGUUCCUGU | 3410 | 1435-1455 | UACAGGAACCUGAGAAAGCACC | 3679 | 1433-1455 |
| | A | | | C | | |
| AD-1735771 | AUGAUACUGAAAGUGGUGAU | 3411 | 3331-3351 | UAUCACCACUUUCAGUAUCAUU | 3680 | 3329-3351 |
| | A | | | A | | |
| AD-1735772 | UCACUGGACCUGAUUUUAGU | 3412 | 2244-2264 | UACUAAAAUCAGGUCCAGUGAU | 3681 | 2242-2264 |
| | A | | | A | | |
| AD-1735773 | GAACUUUUUGCUUUUAUGAA | 3413 | 3991-4011 | UUUCAUAAAAGCAAAAAGUUCC | 3682 | 3989-4011 |
| | A | | | U | | |
| AD-1735774 | GAACACAGCACAGAUUUGUU | 3414 | 4451-4471 | UAACAAAUCUGUGCUGUGUUCA | 3683 | 4449-4471 |
| | A | | | U | | |
| AD-1735775 | AUAUGGAAGGAAAUCGUAGA | 3415 | 3483-3503 | UUCUACGAUUUCCUUCCAUAUC | 3684 | 3481-3503 |
| | A | | | U | | |
| AD-1735776 | GCUAUCUAAAUGGGCCUUUU | 3416 | 1696-1716 | UAAAAGGCCCAUUUAGAUAGCA | 3685 | 1694-1716 |
| | A | | | U | | |
| AD-1735777 | CAAAACCACCAAGAAGGAAA | 3417 | 3409-3429 | UUUUCCUUCUUGGUGGUUUUGG | 3686 | 3407-3429 |
| | A | | | A | | |
| AD-1735778 | CCACAGAUGUGCUACAUGCA | 3418 | 2497-2517 | UUGCAUGUAGCACAUCUGUGGC | 3687 | 2495-2517 |
| | A | | | A | | |
| AD-1735779 | AAAGUCAUACAUGUACAAGC | 3419 | 5444-5464 | UGCUUGUACAUGUAUGACUUUA | 3688 | 5442-5464 |
| | A | | | U | | |
| AD-1735780 | UAGAAGCAGAAGAGGAUAAU | 3420 | 3313-3333 | UAUUAUCCUCUUCUGCUUCUAA | 3689 | 3311-3333 |
| | A | | | C | | |
| AD-1735781 | CGUGUGUUCGCACAGCUUGA | 3421 | 611-631 | UUCAAGCUGUGCGAACACACGC | 3690 | 609-631 |
| | A | | | U | | |
| AD-1735782 | GCUGGUGUUUUGUCCAGUCU | 3422 | 1791-1811 | UAGACUGGACAAAACACCAGCU | 3691 | 1789-1811 |
| | A | | | C | | |
| AD-1735783 | AUGGAAAACAAUCUAAAUCC | 3423 | 3390-3410 | UGGAUUUAGAUUGUUUUCCAUU | 3692 | 3388-3410 |
| | A | | | U | | |
| AD-1735784 | CAUUAGAAAAUGGCUAUCGA | 3424 | 2449-2469 | UUCGAUAGCCAUUUUCUAAUGC | 3693 | 2447-2469 |
| | A | | | A | | |
| AD-1735785 | AUGGACAGAAGGCAUUGUCA | 3425 | 3122-3142 | UUGACAAUGCCUUCUGUCCAUU | 3694 | 3120-3142 |
| | A | | | U | | |
| AD-1735786 | CAGAUUGCUGCAGUGGAAAA | 3426 | 2056-2076 | UUUUUCCACUGCAGCAAUCUGU | 3695 | 2054-2076 |
| | A | | | A | | |
| AD-1735787 | AGGUGGAAAUGGAGUUGAUC | 3427 | 1310-1330 | UGAUCAACUCCAUUUCCACCUC | 3696 | 1308-1330 |
| | A | | | C | | |
| AD-1735788 | CUGGGCAAUGCACCUAAUAC | 3428 | 1935-1955 | UGUAUUAGGUGCAUUGCCCAGA | 3697 | 1933-1955 |
| | A | | | G | | |
| AD-1735789 | AUUUCCCUCUGCCUAAAGAC | 3429 | 4652-4672 | UGUCUUUAGGCAGAGGGAAAUU | 3698 | 4650-4672 |
| | A | | | A | | |
| AD-1735790 | CGCUUCUGCAACGUGGAGCU | 3430 | 429-449 | UAGCUCCACGUUGCAGAAGCGG | 3699 | 427-449 |
| | A | | | C | | |
| AD-1735791 | AGAUUCAUAUUGUAUUUCCC | 3431 | 4014-4034 | UGGGAAAUACAAUAUGAAUCUA | 3700 | 4012-4034 |
| | A | | | U | | |
| AD-1735792 | AGGACAAAUGCAUUUCUAGU | 3432 | 2754-2774 | UACUAGAAAUGCAUUUGUCCUC | 3701 | 2752-2774 |
| | A | | | C | | |
| AD-1735793 | UUAGACGUGUUGAGUGAGUG | 3433 | 5205-5225 | UCACUCACUCAACACGUCUAAU | 3702 | 5203-5225 |
| | A | | | A | | |
| AD-1735794 | CUGUGGACAUCAAAUGCUGA | 3434 | 2006-2026 | UUCAGCAUUUGAUGUCCACAGC | 3703 | 2004-2026 |
| | A | | | U | | |
| AD-1735795 | AGGCCUUAAUACUGUGAGAG | 3435 | 3729-3749 | UCUCUCACAGUAUUAAGGCCUG | 3704 | 3727-3749 |
| | A | | | U | | |
| AD-1735796 | AGAAAUCAUUGCCUAGUGUU | 3436 | 3672-3692 | UAACACUAGGCAAUGAUUUCUG | 3705 | 3670-3692 |
| | A | | | C | | |
| AD-1735797 | UGAGUGCUGAGUUCCUUGCU | 3437 | 5219-5239 | UAGCAAGGAACUCAGCACUCAC | 3706 | 5217-5239 |
| | A | | | U | | |
| AD-1735798 | GCCAUGCAUGAUUAUGAUCA | 3438 | 2727-2747 | UUGAUCAUAAUCAUGCAUGGCA | 3707 | 2725-2747 |
| | A | | | G | | |
| AD-1735799 | UGCUGCUGGUUUGCUACCAG | 3439 | 3284-3304 | UCUGGUAGCAAACCAGCAGCAU | 3708 | 3282-3304 |
| | A | | | C | | |
| AD-1735800 | CAGAUGAAGAGGAAUAGCGA | 3440 | 3595-3615 | UUCGCUAUUCCUCUUCAUCUGC | 3709 | 3593-3615 |
| | A | | | C | | |
| AD-1735801 | AUCCAGGUGCAUCAAUUUCU | 3441 | 4207-4227 | UAGAAAUUGAUGCACCUGGAUU | 3710 | 4205-4227 |
| | A | | | U | | |
| AD-1735802 | AAUAAGCCAAGCGUUAUCUU | 3442 | 1905-1925 | UAAGAUAACGCUUGGCUUAUUA | 3711 | 1903-1925 |
| | A | | | A | | |
| AD-1735803 | GAGAGGAUCCUUGCUCUGCU | 3443 | 3744-3764 | UAGCAGAGCAAGGAUCCUCUCA | 3712 | 3742-3764 |
| | A | | | C | | |
| AD-1735804 | AUGAUGAAGACGGUGAAGAA | 3444 | 3349-3369 | UUUCUUCACCGUCUUCAUCAUC | 3713 | 3347-3369 |
| | A | | | A | | |
| AD-1735805 | UCGUAAGUGUAAGAAAGAAU | 3445 | 5743-5763 | UAUUCUUUCUUACACUUACGAA | 3714 | 5741-5763 |
| | A | | | U | | |
| AD-1735806 | AUCUGUGUAACAGCUGUGGA | 3446 | 1993-2013 | UUCCACAGCUGUUACACAGAUU | 3715 | 1991-2013 |
| | A | | | G | | |
| AD-1735807 | AUUUAAAAUUCCCACUCAAC | 3447 | 2408-2428 | UGUUGAGUGGGAAUUUUAAAUA | 3716 | 2406-2428 |
| | A | | | U | | |
| AD-1735808 | GACCAGUGUCUACUGGCUCU | 3448 | 1837-1857 | UAGAGCCAGUAGACACUGGUCC | 3717 | 1835-1857 |
| | A | | | A | | |
| AD-1735809 | CGCUUUCGUUUUUUAGUCAU | 3449 | 2913-2933 | UAUGACUAAAAAACGAAAGCGC | 3718 | 2911-2933 |
| | A | | | U | | |
| AD-1735810 | UCCGUGUUGACCCAUGUUGC | 3450 | 3829-3849 | UGCAACAUGGGUCAACACGGAU | 3719 | 3827-3849 |
| | A | | | G | | |
| AD-1735811 | AUACAGAAGAUGAAGAAAUG | 3451 | 3373-3393 | UCAUUUCUUCAUCUUCUGUAUC | 3720 | 3371-3393 |
| | A | | | U | | |
| AD-1735812 | UCUCCCGAGCUGAAACUUAA | 3452 | 5719-5739 | UUUAAGUUUCAGCUCGGGAGAG | 3721 | 5717-5739 |
| | A | | | A | | |
| AD-1735813 | UGCAUCAAACUGGCAGAUAU | 3453 | 3066-3086 | UAUAUCUGCCAGUUUGAUGCAC | 3722 | 3064-3086 |
| | A | | | A | | |
| AD-1735814 | UUGCAAAAUAUUCAGGAGAC | 3454 | 1202-1222 | UGUCUCCUGAAUAUUUUGCAAC | 3723 | 1200-1222 |
| | A | | | U | | |
| AD-1735815 | ACAAUCGUAUACAUGCCACA | 3455 | 2482-2502 | UUGUGGCAUGUAUACGAUUGUG | 3724 | 2480-2502 |
| | A | | | A | | |
| AD-1735816 | AAUAGAAAAGAUGAGCAACU | 3456 | 2282-2302 | UAGUUGCUCAUCUUUUCUAUUA | 3725 | 2280-2302 |
| | A | | | A | | |
| AD-1735817 | UAUCUUAUGUUCCAUGGCUG | 3457 | 4325-4345 | UCAGCCAUGGAACAUAAGAUAA | 3726 | 4323-4345 |
| | A | | | G | | |
| AD-1735818 | CUACGUGAAGAGCUGCGUGA | 3458 | 362-382 | UUCACGCAGCUCUUCACGUAGC | 3727 | 360-382 |
| | A | | | C | | |
| AD-1735819 | GAAUUGAUGGCUCUAUACGU | 3459 | 2700-2720 | UACGUAUAGAGCCAUCAAUUCU | 3728 | 2698-2720 |
| | A | | | A | | |
| AD-1735820 | UUCAGGUAAUUGAAGAGGCA | 3460 | 3577-3597 | UUGCCUCUUCAAUUACCUGAAU | 3729 | 3575-3597 |
| | A | | | C | | |
| AD-1735821 | AAGGACUGAGUGACCUUUGU | 3461 | 4174-4194 | UACAAAGGUCACUCAGUCCUUC | 3730 | 4172-4194 |
| | A | | | A | | |
| AD-1735822 | CGAGAGUCUGAGGAACGGCU | 3462 | 344-364 | UAGCCGUUCCUCAGACUCUCGG | 3731 | 342-364 |
| | A | | | G | | |
| AD-1735823 | AUUCUUUGGGAUUGGGACUU | 3463 | 1254-1274 | UAAGUCCCAAUCCCAAAGAAUC | 3732 | 1252-1274 |
| | A | | | A | | |
| AD-1735824 | UACCAGGUCAGUGGUUAGAA | 3464 | 3298-3318 | UUUCUAACCACUGACCUGGUAG | 3733 | 3296-3318 |
| | A | | | C | | |
| AD-1735825 | AUUCAGGUUUGCCAUGGACA | 3465 | 4283-4303 | UUGUCCAUGGCAAACCUGAAUA | 3734 | 4281-4303 |
| | A | | | C | | |
| AD-1735826 | GCAGCUAAACUGGUCCUGGA | 3466 | 34-54 | UUCCAGGACCAGUUUAGCUGCC | 3735 | 32-54 |
| | A | | | G | | |
| AD-1735827 | AGUAAUGGCAUAGAAUGGAG | 3467 | 3012-3032 | UCUCCAUUCUAUGCCAUUACUA | 3736 | 3010-3032 |
| | A | | | U | | |
| AD-1735828 | AAUUGCAAGAAAAUGGACCA | 3468 | 4843-4863 | UUGGUCCAUUUUCUUGCAAUUA | 3737 | 4841-4863 |
| | A | | | U | | |
| AD-1735829 | CGGCACUGCGUUCUGGUGCU | 3469 | 921-941 | UAGCACCAGAACGCAGUGCCGG | 3738 | 919-941 |
| | A | | | A | | |
| AD-1735830 | AGUCAUAUUGAAGAAGCCCA | 3470 | 3633-3653 | UUGGGCUUCUUCAAUAUGACUU | 3739 | 3631-3653 |
| | A | | | U | | |
| AD-1735831 | UCAUCAUGCUGCGUCAGCUU | 3471 | 2828-2848 | UAAGCUGACGCAGCAUGAUGAU | 3740 | 2826-2848 |
| | A | | | U | | |
| AD-1735832 | AAAUGAUCGCCUCUUGGUAU | 3472 | 3038-3058 | UAUACCAAGAGGCGAUCAUUUU | 3741 | 3036-3058 |
| | A | | | C | | |
| AD-1735833 | AUUUGAAGAUAUAAAGAGCA | 3473 | 4922-4942 | UUGCUCUUUAUAUCUUCAAAUG | 3742 | 4920-4942 |
| | A | | | U | | |
| AD-1735834 | GGUCUCCUUCACCAGCCUCG | 3474 | 965-985 | UCGAGGCUGGUGAAGGAGACCC | 3743 | 963-985 |
| | A | | | A | | |
| AD-1735835 | GAAACAGAGAAGAAAGACAG | 3475 | 2142-2162 | UCUGUCUUUCUUCUCUGUUUCC | 3744 | 2140-2162 |
| | A | | | U | | |
| AD-1735836 | UUCUAGUGGCUACAAAUGCC | 3476 | 2767-2787 | UGGCAUUUGUAGCCACUAGAAA | 3745 | 2765-2787 |
| | A | | | U | | |
| AD-1735837 | GGAGAGAAAUCAGGAAGGAU | 3477 | 2337-2357 | UAUCCUUCCUGAUUUCUCUCCC | 3746 | 2335-2357 |
| | A | | | A | | |
| AD-1735838 | AUAUAGUGAGCCCUAAAGGA | 3478 | 4533-4553 | UUCCUUUAGGGCUCACUAUAUU | 3747 | 4531-4553 |
| | A | | | U | | |
| AD-1735839 | GCUGCCUUUGUCCUCGCCCU | 3479 | 528-548 | UAGGGCGAGGACAAAGGCAGCC | 3748 | 526-548 |
| | A | | | A | | |
| AD-1735840 | GGCAAAAGACCUCACCAAGA | 3480 | 1653-1673 | UUCUUGGUGAGGUCUUUUGCCA | 3749 | 1651-1673 |
| | A | | | U | | |
| AD-1735841 | UUCAGCAUCGCCUGUGCCUU | 3481 | 639-659 | UAAGGCACAGGCGAUGCUGAAG | 3750 | 637-659 |
| | A | | | A | | |
| AD-1735842 | GCUAAAUGAGGCUCGCAAUA | 3482 | 1337-1357 | UUAUUGCGAGCCUCAUUUAGCA | 3751 | 1335-1357 |
| | A | | | C | | |
| AD-1735843 | CAGCUUCGUCUGGUGGGUCU | 3483 | 950-970 | UAGACCCACCAGACGAAGCUGG | 3752 | 948-970 |
| | A | | | C | | |
| AD-1735844 | AUAUGGUAUUGGCAUGCAGU | 3484 | 5066-5086 | UACUGCAUGCCAAUACCAUAUU | 3753 | 5064-5086 |
| | A | | | C | | |
| AD-1735845 | AAACUGCAGGUGGAGAAUUC | 3485 | 3534-3554 | UGAAUUCUCCACCUGCAGUUUA | 3754 | 3532-3554 |
| | A | | | U | | |
| AD-1735846 | UAUGGCUGCCUGUCUUCAAA | 3486 | 2667-2687 | UUUUGAAGACAGGCAGCCAUAA | 3755 | 2665-2687 |
| | A | | | C | | |
| AD-1735847 | CUGUUCUGAAAUAGAGGACC | 3487 | 1499-1519 | UGGUCCUCUAUUUCAGAACAGG | 3756 | 1497-1519 |
| | A | | | G | | |
| AD-1735848 | UCUAUUUUUAAAGCCGCUGG | 3488 | 5898-5918 | UCCAGCGGCUUUAAAAAUAGAA | 3757 | 5896-5918 |
| | A | | | A | | |
| AD-1735849 | GCUGAGGAGAAGCUCAGGAA | 3489 | 1583-1603 | UUUCCUGAGCUUCUCCUCAGCU | 3758 | 1581-1603 |
| | A | | | G | | |
| AD-1735850 | CAAGCCUUCCACCACAAGUC | 3490 | 1381-1401 | UGACUUGUGGUGGAAGGCUUGG | 3759 | 1379-1401 |
| | A | | | A | | |
| AD-1735851 | UUCAGUUACCAAAGUGGCCA | 3491 | 3970-3990 | UUGGCCACUUUGGUAACUGAAA | 3760 | 3968-3990 |
| | A | | | U | | |
| AD-1735852 | CACCUGCUUCCUCACCCGGA | 3492 | 668-688 | UUCCGGGUGAGGAAGCAGGUGA | 3761 | 666-688 |
| | A | | | G | | |
| AD-1735853 | UUUUAUCACCCACAUAGUGG | 3493 | 3242-3262 | UCCACUAUGUGGGUGAUAAAAG | 3762 | 3240-3262 |
| | A | | | A | | |
| AD-1735854 | AGGAGACCGUCGUUGCCUUG | 3494 | 1215-1235 | UCAAGGCAACGACGGUCUCCUG | 3763 | 1213-1235 |
| | A | | | A | | |
| AD-1735855 | AUGGCCCAGCAAAAGUUCGA | 3495 | 3088-3108 | UUCGAACUUUUGCUGGGCCAUU | 3764 | 3086-3108 |
| | A | | | U | | |
| AD-1735856 | AGACGCCAAAGCCAUGCGGU | 3496 | 290-310 | UACCGCAUGGCUUUGGCGUCUC | 3765 | 288-310 |
| | A | | | G | | |
| AD-1735857 | CAGCUAAUGCACCACCUCAC | 3497 | 3450-3470 | UGUGAGGUGGUGCAUUAGCUGA | 3766 | 3448-3470 |
| | A | | | C | | |
| AD-1735858 | GGAAAAGCAGACGGCGAAUA | 3498 | 3424-3444 | UUAUUCGCCGUCUGCUUUUCCU | 3767 | 3422-3444 |
| | A | | | U | | |
| AD-1735859 | GCUUUGCUGGGUAGUGAGCU | 3499 | 3901-3921 | UAGCUCACUACCCAGCAAAGCA | 3768 | 3899-3921 |
| | A | | | G | | |
| AD-1735860 | AAAUGAAACAGAUUCUGAUG | 4576 | 2582-2602 | UCAUCAGAAUCUGUUUCAUUUC | 4577 | 2580-2602 |
| | A | | | C | | |

TABLE 15
Modified Sense and Antisense Strand Sequences of PDE3B dsRNA Agents
Comprising a GalNAc Derivative Targeting Ligand

| | | SEQ | | SEQ | | SEQ |
|---|---|---|---|---|---|---|
| Duplex | Sense Sequence | ID | Antisense Sequence | ID | mRNA Target | ID |
| Name | 5′ to 3′ | NO: | 5′ to 3′ | NO: | Sequence 5′ to 3′ | NO: |
| AD- | ususccuuCfuUfCfAfucuugau | 4578 | VPusUfsgauCfaAfGfaugaAfgAfagga | 4038 | ACUUCCUUCUUCAUCUUGA | 4307 |
| 1735591 | csasa | | asgsu | | UCAU | |
| AD- | csascucuAfaAfAfCfugauguu | 4579 | VPusUfsgaaCfaUfCfaguuUfuAfgagu | 4039 | UUCACUCUAAAACUGAUGU | 4308 |
| 1735592 | csasa | | gsasa | | UCAU | |
| AD- | uscsuucuGfuAfUfCfacugacu | 4580 | VPusUfsgagUfcAfGfugauAfcAfgaag | 4040 | CAUCUUCUGUAUCACUGAC | 4309 |
| 1735593 | csasa | | asusg | | UCAC | |
| AD- | csasgaucUfuCfUfGfacugauc | 4581 | VPusUfsggaUfcAfGfucagAfaGfaucu | 4041 | GUCAGAUCUUCUGACUGAU | 4310 |
| 1735594 | csasa | | gsasc | | CCAA | |
| AD- | usgsacacAfuUfUfUfucuccau | 4582 | VPusUfsuauGfgAfGfaaaaAfuGfuguc | 4042 | ACUGACACAUUUUUCUCCA | 4311 |
| 1735595 | asasa | | asgsu | | UAAA | |
| AD- | gsgsaaacUfcAfUfUfuuuacau | 4583 | VPusUfsuauGfuAfAfaaauGfaGfuuuc | 4043 | CUGGAAACUCAUUUUUACA | 4312 |
| 1735596 | asasa | | csasg | | UAAU | |
| AD- | asuscagcAfaCfUfUfagaaauu | 4584 | VPusAfsgaaUfuUfCfuaagUfuGfcuga | 4044 | UUAUCAGCAACUUAGAAAU | 4313 |
| 1735597 | csusa | | usasa | | UCUG | |
| AD- | csusuauaUfuUfCfUfcuagagu | 4585 | VPusGfsuacUfcUfAfgagaAfaUfauaa | 4045 | UCCUUAUAUUUCUCUAGAG | 4314 |
| 1735598 | ascsa | | gsgsa | | UACA | |
| AD- | asascuccAfaGfAfAfucuuuua | 4586 | VPusGfsauaAfaAfGfauucUfuGfgagu | 4046 | AAAACUCCAAGAAUCUUUU | 4315 |
| 1735599 | uscsa | | ususu | | AUCA | |
| AD- | csusucaaAfcAfUfUfccugcau | 4587 | VPusUfsaauGfcAfGfgaauGfuUfugaa | 4047 | GUCUUCAAACAUUCCUGCA | 4316 |
| 1735600 | usasa | | gsasc | | UUAG | |
| AD- | uscsguucUfuCfUfCfcucaacu | 4588 | VPusCfsuagUfuGfAfggagAfaGfaacg | 4048 | GAUCGUUCUUCUCCUCAAC | 4317 |
| 1735601 | asgsa | | asusc | | UAGC | |
| AD- | asasagauUfaAfUfCfcucucac | 4589 | VPusGfsuguGfaGfAfggauUfaAfucuu | 4049 | CCAAAGAUUAAUCCUCUCA | 4318 |
| 1735602 | ascsa | | usgsg | | CACC | |
| AD- | ascsuaacCfuCfAfUfcaaagau | 4590 | VPusGfsuauCfuUfUfgaugAfgGfuuag | 4050 | AAACUAACCUCAUCAAAGA | 4319 |
| 1735603 | ascsa | | ususu | | UACA | |
| AD- | csasccacUfuAfUfUfacuaaua | 4591 | VPusUfsuuaUfuAfGfuaauAfaGfuggu | 4051 | CCCACCACUUAUUACUAAU | 4320 |
| 1735604 | asasa | | gsgsg | | AAAA | |
| AD- | usgsguacUfaGfAfAfauauucu | 4592 | VPusAfsaagAfaUfAfuuucUfaGfuacc | 4052 | GCUGGUACUAGAAAUAUUC | 4321 |
| 1735605 | ususa | | asgsc | | UUUU | |
| AD- | gscsugucAfuUfUfGfuucacuc | 4593 | VPusUfsagaGfuGfAfacaaAfuGfacag | 4053 | UAGCUGUCAUUUGUUCACU | 4322 |
| 1735606 | usasa | | csusa | | CUAA | |
| AD- | ascsucaaCfaAfUfUfuaugaac | 4594 | VPusUfsaguUfcAfUfaaauUfgUfugag | 4054 | CCACUCAACAAUUUAUGAA | 4323 |
| 1735607 | usasa | | usgsg | | CUAU | |
| AD- | asgsuaugAfcUfCfAfuuaauag | 4595 | VPusUfsucuAfuUfAfaugaGfuCfauac | 4055 | AGAGUAUGACUCAUUAAUA | 4324 |
| 1735608 | asasa | | uscsu | | GAAA | |
| AD- | csusgcugAfuUfUfUfcuuaaua | 4596 | VPusCfsuuaUfuAfAfgaaaAfuCfagca | 4056 | UACUGCUGAUUUUCUUAAU | 4325 |
| 1735609 | asgsa | | gsusa | | AAGC | |
| AD- | usasucuuUfaGfAfUfccacauc | 4597 | VPusAfsagaUfgUfGfgaucUfaAfagau | 4057 | GUUAUCUUUAGAUCCACAU | 4326 |
| 1735610 | ususa | | asasc | | CUUA | |
| AD- | csgsagacAfuUfCfCfuuaucac | 4598 | VPusUfsuguGfaUfAfaggaAfuGfucuc | 4058 | AUCGAGACAUUCCUUAUCA | 4327 |
| 1735611 | asasa | | gsasu | | CAAU | |
| AD- | usascugaAfuCfAfGfauuaauu | 4599 | VPusGfsaaaUfuAfAfucugAfuUfcagu | 4059 | GUUACUGAAUCAGAUUAAU | 4328 |
| 1735612 | uscsa | | asasc | | UUCU | |
| AD- | csusuacuUfaAfAfUfccuucac | 4600 | VPusCfsaguGfaAfGfgauuUfaAfguaa | 4060 | CCCUUACUUAAAUCCUUCA | 4329 |
| 1735613 | usgsa | | gsgsg | | CUGG | |
| AD- | uscsgaaaGfaAfUfCfauucaaa | 4601 | VPusAfsguuUfgAfAfugauUfcUfuucg | 4061 | UCUCGAAAGAAUCAUUCAA | 4330 |
| 1735614 | csusa | | asgsa | | ACUU | |
| AD- | asasaugaCfcUfAfUfcacuacu | 4602 | VPusUfsaagUfaGfUfgauaGfgUfcauu | 4062 | AGAAAUGACCUAUCACUAC | 4331 |
| 1735615 | usasa | | uscsu | | UUAU | |
| AD- | csasagucAfuUfUfCfcucucua | 4603 | VPusCfsguaGfaGfAfggaaAfuGfacuu | 4063 | CACAAGUCAUUUCCUCUCU | 4332 |
| 1735616 | csgsa | | gsusg | | ACGG | |
| AD- | asusgcagUfuUfCfUfuacuuau | 4604 | VPusAfsgauAfaGfUfaagaAfaCfugca | 4064 | GCAUGCAGUUUCUUACUUA | 4333 |
| 1735617 | csusa | | usgsc | | UCUA | |
| AD- | uscsaaugCfaAfUfAfcacuguu | 4605 | VPusUfsgaaCfaGfUfguauUfgCfauug | 4065 | GCUCAAUGCAAUACACUGU | 4334 |
| 1735618 | csasa | | asgsc | | UCAG | |
| AD- | uscsuaugUfaCfCfUfacugaca | 4606 | VPusUfsgugUfcAfGfuaggUfaCfauag | 4066 | UAUCUAUGUACCUACUGAC | 4335 |
| 1735619 | csasa | | asusa | | ACAU | |
| AD- | usgsaauuCfcAfGfUfcuuaucu | 4607 | VPusUfsaagAfuAfAfgacuGfgAfauuc | 4067 | UUUGAAUUCCAGUCUUAUC | 4336 |
| 1735620 | usasa | | asasa | | UUAU | |
| AD- | ususgcugUfaAfUfAfccaaaac | 4608 | VPusUfsaguUfuUfGfguauUfaCfagca | 4068 | AUUUGCUGUAAUACCAAAA | 4337 |
| 1735621 | usasa | | asasu | | CUAA | |
| AD- | gsgsaucaCfuUfCfUfuucaaau | 4609 | VPusUfsgauUfuGfAfaagaAfgUfgauc | 4069 | UUGGAUCACUUCUUUCAAA | 4338 |
| 1735622 | csasa | | csasa | | UCAG | |
| AD- | gsasacuaGfaAfAfAfcuauucu | 4610 | VPusUfsaagAfaUfAfguuuUfcUfaguu | 4070 | AGGAACUAGAAAACUAUUC | 4339 |
| 1735623 | usasa | | cscsu | | UUAA | |
| AD- | ascsauuuCfcAfUfAfcugucug | 4611 | VPusAfsacaGfaCfAfguauGfgAfaaug | 4071 | UCACAUUUCCAUACUGUCU | 4340 |
| 1735624 | ususa | | usgsa | | GUUA | |
| AD- | usgsauccUfuCfAfCfaguguca | 4612 | VPusAfsuugAfcAfCfugugAfaGfgauc | 4072 | UUUGAUCCUUCACAGUGUC | 4341 |
| 1735625 | asusa | | asasa | | AAUA | |
| AD- | gsasuuucUfgAfGfAfacaagua | 4613 | VPusUfsuuaCfuUfGfuucuCfaGfaaau | 4073 | CUGAUUUCUGAGAACAAGU | 4342 |
| 1735626 | asasa | | csasg | | AAAG | |
| AD- | cscsuacuUfgUfGfAfaauacau | 4614 | VPusAfsaauGfuAfUfuucaCfaAfguag | 4074 | ACCCUACUUGUGAAAUACA | 4343 |
| 1735627 | ususa | | gsgsu | | UUUG | |
| AD- | csasacugGfaAfUfUfuuccaau | 4615 | VPusAfsaauUfgGfAfaaauUfcCfaguu | 4075 | AGCAACUGGAAUUUUCCAA | 4344 |
| 1735628 | ususa | | gscsu | | UUUU | |
| AD- | uscsuaauCfcUfGfAfugagagu | 4616 | VPusUfsaacUfcUfCfaucaGfgAfuuag | 4076 | GCUCUAAUCCUGAUGAGAG | 4345 |
| 1735629 | usasa | | asgsc | | UUAU | |
| AD- | asgsccucAfuUfAfUfcaaaauu | 4617 | VPusAfsgaaUfuUfUfgauaAfuGfaggc | 4077 | UAAGCCUCAUUAUCAAAAU | 4346 |
| 1735630 | csusa | | ususa | | UCUG | |
| AD- | asasuuucUfgAfUfGfcuuuuua | 4618 | VPusAfsguaAfaAfAfgcauCfaGfaaau | 4078 | UCAAUUUCUGAUGCUUUUU | 4347 |
| 1735631 | csusa | | usgsa | | ACUA | |
| AD- | usasccauUfaGfAfUfgaaaucu | 4619 | VPusUfsaagAfuUfUfcaucUfaAfuggu | 4079 | UGUACCAUUAGAUGAAAUC | 4348 |
| 1735632 | usasa | | ascsa | | UUAC | |
| AD- | csusgugaAfuUfCfUfuccaacc | 4620 | VPusAfsuggUfuGfGfaagaAfuUfcaca | 4080 | UCCUGUGAAUUCUUCCAAC | 4349 |
| 1735633 | asusa | | gsgsa | | CAUG | |
| AD- | csusaauaCfuCfCfAfgauuuuu | 4621 | VPusAfsuaaAfaAfUfcuggAfgUfauua | 4081 | ACCUAAUACUCCAGAUUUU | 4350 |
| 1735634 | asusa | | gsgsu | | UAUC | |
| AD- | gsgsacacAfuAfCfAfuugaaua | 4622 | VPusUfsuuaUfuCfAfauguAfuGfuguc | 4082 | AAGGACACAUACAUUGAAU | 4351 |
| 1735635 | asasa | | csusu | | AAAU | |
| AD- | ususugucAfuAfAfUfgcugcuu | 4623 | VPusCfsaaaGfcAfGfcauuAfuGfacaa | 4083 | ACUUUGUCAUAAUGCUGCU | 4352 |
| 1735636 | usgsa | | asgsu | | UUGC | |
| AD- | ususcaucUfuUfCfUfguuaauu | 4624 | VPusGfsaaaUfuAfAfcagaAfaGfauga | 4084 | UGUUCAUCUUUCUGUUAAU | 4353 |
| 1735637 | uscsa | | ascsa | | UUCC | |
| AD- | csasgugaAfaCfCfAfuuaauuu | 4625 | VPusGfsaaaAfuUfAfauggUfuUfcacu | 4085 | CACAGUGAAACCAUUAAUU | 4354 |
| 1735638 | uscsa | | gsusg | | UUCC | |
| AD- | usasacugCfaAfGfAfcugauuu | 4626 | VPusAfsgaaAfuCfAfgucuUfgCfaguu | 4086 | CCUAACUGCAAGACUGAUU | 4355 |
| 1735639 | csusa | | asgsg | | UCUG | |
| AD- | asgsaauuCfcUfCfCfuuaccuc | 4627 | VPusUfsugaGfgUfAfaggaGfgAfauuc | 4087 | GGAGAAUUCCUCCUUACCU | 4356 |
| 1735640 | asasa | | uscsc | | CAAG | |
| AD- | usasuuacAfgAfUfCfuuacuuu | 4628 | VPusAfscaaAfgUfAfagauCfuGfuaau | 4088 | CAUAUUACAGAUCUUACUU | 4357 |
| 1735641 | gsusa | | asusg | | UGUU | |
| AD- | ususaucuGfaAfAfGfauaucaa | 4629 | VPusAfsauuGfaUfAfucuuUfcAfgaua | 4089 | GCUUAUCUGAAAGAUAUCA | 4358 |
| 1735642 | ususa | | asgsc | | AUUU | |
| AD- | asgsaucuGfuUfCfUfggaaaau | 4630 | VPusUfsgauUfuUfCfcagaAfcAfgauc | 4090 | ACAGAUCUGUUCUGGAAAA | 4359 |
| 1735643 | csasa | | usgsu | | UCAU | |
| AD- | cscscagaAfuAfCfAfacuuccu | 4631 | VPusGfsaagGfaAfGfuuguAfuUfcugg | 4091 | CGCCCAGAAUACAACUUCC | 4360 |
| 1735644 | uscsa | | gscsg | | UUCU | |
| AD- | gsusugauCfuUfUfCfagugcua | 4632 | VPusUfsuuaGfcAfCfugaaAfgAfucaa | 4092 | GAGUUGAUCUUUCAGUGCU | 4361 |
| 1735645 | asasa | | csusc | | AAAU | |
| AD- | csusaagcUfuUfAfUfuuauuag | 4633 | VPusGfsucuAfaUfAfaauaAfaGfcuua | 4093 | CACUAAGCUUUAUUUAUUA | 4362 |
| 1735646 | ascsa | | gsusg | | GACG | |
| AD- | asgscuugGfaAfUfCfuauaucu | 4634 | VPusAfsaagAfuAfUfagauUfcCfaagc | 4094 | UCAGCUUGGAAUCUAUAUC | 4363 |
| 1735647 | ususa | | usgsa | | UUUC | |
| AD- | ususauuaUfuUfCfUfgaagccu | 4635 | VPusUfsuagGfcUfUfcagaAfaUfaaua | 4095 | ACUUAUUAUUUCUGAAGCC | 4364 |
| 1735648 | asasa | | asgsu | | UAAC | |
| AD- | cscsuuugUfuGfUfAfuuuaaca | 4636 | VPusUfsuugUfuAfAfauacAfaCfaaag | 4096 | GACCUUUGUUGUAUUUAAC | 4365 |
| 1735649 | asasa | | gsusc | | AAAA | |
| AD- | gscsucucUfaAfCfUfaaucgau | 4637 | VPusUfsgauCfgAfUfuaguUfaGfagag | 4097 | UGGCUCUCUAACUAAUCGA | 4366 |
| 1735650 | csasa | | cscsa | | UCAC | |
| AD- | asasagacUfaCfAfUfgacagaa | 4638 | VPusAfsuuuCfuGfUfcaugUfaGfucuu | 4098 | CUAAAGACUACAUGACAGA | 4367 |
| 1735651 | asusa | | usasg | | AAUG | |
| AD- | csasggauUfcGfUfAfuuuuuaa | 4639 | VPusCfsuuuAfaAfAfauacGfaAfuccu | 4099 | CUCAGGAUUCGUAUUUUUA | 4368 |
| 1735652 | asgsa | | gsasg | | AAGA | |
| AD- | cscsuugcUfaAfGfUfaauugac | 4640 | VPusAfsuguCfaAfUfuacuUfaGfcaag | 4100 | ACCCUUGCUAAGUAAUUGA | 4369 |
| 1735653 | asusa | | gsgsu | | CAUA | |
| AD- | cscsuaugCfaCfUfUfucacagg | 4641 | VPusUfsuccUfgUfGfaaagUfgCfauag | 4101 | CUCCUAUGCACUUUCACAG | 4370 |
| 1735654 | asasa | | gsasg | | GAAC | |
| AD- | asusgcugAfaAfUfAfuguuuca | 4642 | VPusGfsuugAfaAfCfauauUfuCfagca | 4102 | AAAUGCUGAAAUAUGUUUC | 4371 |
| 1735655 | ascsa | | ususu | | AACA | |
| AD- | cscsuuuuAfaUfUfCfaaaucua | 4643 | VPusAfsguaGfaUfUfugaaUfuAfaaag | 4103 | GGCCUUUUAAUUCAAAUCU | 4372 |
| 1735656 | csusa | | gscsc | | ACUG | |
| AD- | ascsuggaCfuUfAfUfuggauuu | 4644 | VPusUfsuaaAfuCfCfaauaAfgUfccag | 4104 | AUACUGGACUUAUUGGAUU | 4373 |
| 1735657 | asasa | | usasu | | UAAU | |
| AD- | csasucugAfaUfCfAfgauggua | 4645 | VPusUfsguaCfcAfUfcugaUfuCfagau | 4105 | AACAUCUGAAUCAGAUGGU | 4374 |
| 1735658 | csasa | | gsusu | | ACAG | |
| AD- | asasuucuGfaUfAfGfcaaucug | 4646 | VPusCfsacaGfaUfUfgcuaUfcAfgaau | 4106 | GAAAUUCUGAUAGCAAUCU | 4375 |
| 1735659 | usgsa | | ususc | | GUGU | |
| AD- | uscsaaguCfaAfGfGfaugcuau | 4647 | VPusAfsgauAfgCfAfuccuUfgAfcuug | 4107 | UUUCAAGUCAAGGAUGCUA | 4376 |
| 1735660 | csusa | | asasa | | UCUA | |
| AD- | uscsacugAfaAfAfCfcacaaga | 4648 | VPusUfsaucUfuGfUfgguuUfuCfagug | 4108 | CCUCACUGAAAACCACAAG | 4377 |
| 1735661 | usasa | | asgsg | | AUAU | |
| AD- | ascscuguUfgAfAfCfagucuuc | 4649 | VPusUfsugaAfgAfCfuguuCfaAfcagg | 4109 | CUACCUGUUGAACAGUCUU | 4378 |
| 1735662 | asasa | | usasg | | CAAG | |
| AD- | uscsaaguGfaCfAfUfauuucag | 4650 | VPusAfsacuGfaAfAfuaugUfcAfcuug | 4110 | ACUCAAGUGACAUAUUUCA | 4379 |
| 1735663 | ususa | | asgsu | | GUUA | |
| AD- | uscsaggaAfcUfUfCfaggauug | 4651 | VPusAfsgcaAfuCfCfugaaGfuUfccug | 4111 | GCUCAGGAACUUCAGGAUU | 4380 |
| 1735664 | csusa | | asgsc | | GCUA | |
| AD- | ascsggagUfaUfUfAfguagcuu | 4652 | VPusUfsuaaGfcUfAfcuaaUfaCfuccg | 4112 | CUACGGAGUAUUAGUAGCU | 4381 |
| 1735665 | asasa | | usasg | | UAAU | |
| AD- | asusugucAfaUfGfAfauuuuau | 4653 | VPusUfscauAfaAfAfuucaUfuGfacaa | 4113 | GCAUUGUCAAUGAAUUUUA | 4382 |
| 1735666 | gsasa | | usgsc | | UGAG | |
| AD- | gsasacagCfaAfAfCfaaauauu | 4654 | VPusUfscaaUfaUfUfuguuUfgCfuguu | 4114 | GUGAACAGCAAACAAAUAU | 4383 |
| 1735667 | gsasa | | csasc | | UGAA | |
| AD- | usgsaacuAfuUfUfUfcgugcau | 4655 | VPusUfsaauGfcAfCfgaaaAfuAfguuc | 4115 | UAUGAACUAUUUUCGUGCA | 4384 |
| 1735668 | usasa | | asusa | | UUAG | |
| AD- | ascsuggaAfuAfCfUfUauuuuu | 4656 | VPusUfsgaaAfaAfUfaaguAfuUfccag | 4116 | GAACUGGAAUACUUAUUUU | 4385 |
| 1735669 | csasa | | ususc | | UCAU | |
| AD- | ususaucuUfgCfAfGfagaucuc | 4657 | VPusCfsagaGfaUfCfucugCfaAfgaua | 4117 | CGUUAUCUUGCAGAGAUCU | 4386 |
| 1735670 | usgsa | | ascsg | | CUGG | |
| AD- | gsasagugUfuAfGfAfauuuuu | 4658 | VPusAfsucaAfaAfAfuucuAfaCfacuu | 4118 | UGGAAGUGUUAGAAUUUUU | 4387 |
| 1735671 | gasusa | | cscsa | | GAUC | |
| AD- | uscsaauuUfaAfGfAfucucugg | 4659 | VPusUfsuccAfgAfGfaucuUfaAfauug | 4119 | UAUCAAUUUAAGAUCUCUG | 4388 |
| 1735672 | asasa | | asusa | | GAAG | |
| AD- | usgsgaccAfuAfUfUfuacaaug | 4660 | VPusAfsacaUfuGfUfaaauAfuGfgucc | 4120 | AAUGGACCAUAUUUACAAU | 4389 |
| 1735673 | ususa | | asusu | | GUUU | |
| AD- | usasgucaUfuGfAfAfgcaaucc | 4661 | VPusAfsaggAfuUfGfcuucAfaUfgacu | 4121 | UUUAGUCAUUGAAGCAAUC | 4390 |
| 1735674 | ususa | | asasa | | CUUG | |
| AD- | asgscuguUfuAfGfGfuuuaaca | 4662 | VPusAfsuugUfuAfAfaccuAfaAfcagc | 4122 | AAAGCUGUUUAGGUUUAAC | 4391 |
| 1735675 | asusa | | ususu | | AAUG | |
| AD- | csasaggcAfaAfUfGfauguaaa | 4663 | VPusUfsauuUfaCfAfucauUfuGfccuu | 4123 | GCCAAGGCAAAUGAUGUAA | 4392 |
| 1735676 | usasa | | gsgsc | | AUAG | |
| AD- | asusccaaUfuAfCfUfacuggaa | 4664 | VPusGfsuuuCfcAfGfuaguAfaUfugga | 4124 | AAAUCCAAUUACUACUGGA | 4393 |
| 1735677 | ascsa | | ususu | | AACU | |
| AD- | ascsuuacAfaUfAfUfauauauc | 4665 | VPusAfsggaUfaUfAfuauaUfuGfuaag | 4125 | GCACUUACAAUAUAUAUAU | 4394 |
| 1735678 | csusa | | usgsc | | CCUG | |
| AD- | asusgcagUfuUfGfGfuaucuga | 4666 | VPusUfsgucAfgAfUfaccaAfaCfugca | 4126 | ACAUGCAGUUUGGUAUCUG | 4395 |
| 1735679 | csasa | | usgsu | | ACAA | |
| AD- | cscsuuauUfuCfAfAfgacacug | 4667 | VPusAfsccaGfuGfUfcuugAfaAfuaag | 4127 | UACCUUAUUUCAAGACACU | 4396 |
| 1735680 | gsusa | | gsusa | | GGUU | |
| AD- | gsasagggAfuUfUfAfuucuuua | 4668 | VPusAfscuaAfaGfAfauaaAfuCfccuu | 4128 | AUGAAGGGAUUUAUUCUUU | 4397 |
| 1735681 | gsusa | | csasu | | AGUC | |
| AD- | asusccuuGfcUfAfCfggaucuu | 4669 | VPusUfsuaaGfaUfCfcguaGfcAfagga | 4129 | CAAUCCUUGCUACGGAUCU | 4398 |
| 1735682 | asasa | | ususg | | UAAA | |
| AD- | ascsugacUfaUfCfCfcgaagca | 4670 | VPusUfsuugCfuUfCfgggaUfaGfucag | 4130 | CUACUGACUAUCCCGAAGC | 4399 |
| 1735683 | asasa | | usasg | | AAAG | |
| AD- | usgsuauuUfcUfGfCfuauuauu | 4671 | VPusGfsaaaUfaAfUfagcaGfaAfauac | 4131 | AGUGUAUUUCUGCUAUUAU | 4400 |
| 1735684 | uscsa | | ascsu | | UUCC | |
| AD- | usgsuaacAfaUfAfAfcuagccu | 4672 | VPusUfsuagGfcUfAfguuaUfuGfuuac | 4132 | UAUGUAACAAUAACUAGCC | 4401 |
| 1735685 | asasa | | asusa | | UAAA | |
| AD- | gsusuguuUfuUfAfCfucuagcu | 4673 | VPusUfsgagCfuAfGfaguaAfaAfacaa | 4133 | UUGUUGUUUUUACUCUAGC | 4402 |
| 1735686 | csasa | | csasa | | UCAG | |
| AD- | csasggcaGfuUfUfUfauacaau | 4674 | VPusUfscauUfgUfAfuaaaAfcUfgccu | 4134 | CUCAGGCAGUUUUAUACAA | 4403 |
| 1735687 | gsasa | | gsasg | | UGAC | |
| AD- | asgsgauuCfuCfAfGfucagguu | 4675 | VPusAfsuaaCfcUfGfacugAfgAfaucc | 4135 | GAAGGAUUCUCAGUCAGGU | 4404 |
| 1735688 | asusa | | ususc | | UAUG | |
| AD- | asusuuucCfaUfCfAfuguuuaa | 4676 | VPusAfscuuAfaAfCfaugaUfgGfaaaa | 4136 | ACAUUUUCCAUCAUGUUUA | 4405 |
| 1735689 | gsusa | | usgsu | | AGUG | |
| AD- | usgsccacUfuUfUfGfuuaccau | 4677 | VPusCfsaauGfgUfAfacaaAfaGfuggc | 4137 | GCUGCCACUUUUGUUACCA | 4406 |
| 1735690 | usgsa | | asgsc | | UUGU | |
| AD- | ascsuugcUfaGfUfGfuguggau | 4678 | VPusAfsuauCfcAfCfacacUfaGfcaag | 4138 | AAACUUGCUAGUGUGUGGA | 4407 |
| 1735691 | asusa | | ususu | | UAUG | |
| AD- | asasucuuAfcUfUfGfagaaauu | 4679 | VPusGfscaaUfuUfCfucaaGfuAfagau | 4139 | GAAAUCUUACUUGAGAAAU | 4408 |
| 1735692 | gscsa | | ususc | | UGCC | |
| AD- | asgsuucgAfgAfCfUfugcauuu | 4680 | VPusUfscaaAfuGfCfaaguCfuCfgaac | 4140 | AAAGUUCGAGACUUGCAUU | 4409 |
| 1735693 | gsasa | | ususu | | UGAA | |
| AD- | cscsauagAfaUfUfUfccugaua | 4681 | VPusAfsguaUfcAfGfgaaaUfuCfuaug | 4141 | ACCCAUAGAAUUUCCUGAU | 4410 |
| 1735694 | csusa | | gsgsu | | ACUG | |
| AD- | asgsguaaUfuCfCfUfuuagaau | 4682 | VPusAfsuauUfcUfAfaaggAfaUfuacc | 4142 | CAAGGUAAUUCCUUUAGAA | 4411 |
| 1735695 | asusa | | ususg | | UAUG | |
| AD- | csusuaugGfaAfAfCfucaacaa | 4683 | VPusUfscuuGfuUfGfaguuUfcCfauaa | 4143 | AACUUAUGGAAACUCAACA | 4412 |
| 1735696 | gsasa | | gsusu | | AGAA | |
| AD- | asasgcagCfaUfUfUfuaaauua | 4684 | VPusCfsguaAfuUfUfaaaaUfgCfugcu | 4144 | AAAAGCAGCAUUUUAAAUU | 4413 |
| 1735697 | csgsa | | ususu | | ACGA | |
| AD- | usgsacucUfcAfAfCfugaccau | 4685 | VPusGfsaauGfgUfCfaguuGfaGfaguc | 4145 | GCUGACUCUCAACUGACCA | 4414 |
| 1735698 | uscsa | | asgsc | | UUCC | |
| AD- | asgsguuuAfuCfCfAfugaagga | 4686 | VPusAfsgucCfuUfCfauggAfuAfaacc | 4146 | CUAGGUUUAUCCAUGAAGG | 4415 |
| 1735699 | csusa | | usasg | | ACUG | |
| AD- | ascscaagAfaUfUfUfggcauuu | 4687 | VPusUfsgaaAfuGfCfcaaaUfuCfuugg | 4147 | UCACCAAGAAUUUGGCAUU | 4416 |
| 1735700 | csasa | | usgsa | | UCAA | |
| AD- | usasucuaGfaAfUfAfuuuggcu | 4688 | VPusUfsaagCfcAfAfauauUfcUfagau | 4148 | CUUAUCUAGAAUAUUUGGC | 4417 |
| 1735701 | usasa | | asasg | | UUAU | |
| AD- | csusccugCfaGfAfAfuacauac | 4689 | VPusUfsuguAfuGfUfauucUfgCfagga | 4149 | CUCUCCUGCAGAAUACAUA | 4418 |
| 1735702 | asasa | | gsasg | | CAAG | |
| AD- | uscsacacCfaUfUfUfccuggau | 4690 | VPusAfsaauCfcAfGfgaaaUfgGfugug | 4150 | UCUCACACCAUUUCCUGGA | 4419 |
| 1735703 | ususa | | asgsa | | UUUU | |
| AD- | asusguggAfaUfUfCfaagcgcu | 4691 | VPusAfsaagCfgCfUfugaaUfuCfcaca | 4151 | UCAUGUGGAAUUCAAGCGC | 4420 |
| 1735704 | ususa | | usgsa | | UUUC | |
| AD- | asusugugUfaUfUfAfucuacua | 4692 | VPusCfsauaGfuAfGfauaaUfaCfacaa | 4152 | CUAUUGUGUAUUAUCUACU | 4421 |
| 1735705 | usgsa | | usasg | | AUGU | |
| AD- | asasuggaGfuAfAfUfgaaaaug | 4693 | VPusAfsucaUfuUfUfcauuAfcUfccau | 4153 | AGAAUGGAGUAAUGAAAAU | 4422 |
| 1735706 | asusa | | uscsu | | GAUC | |
| AD- | ascsugguUfuAfUfUfggaaaua | 4694 | VPusAfsauaUfuUfCfcaauAfaAfccag | 4154 | ACACUGGUUUAUUGGAAAU | 4423 |
| 1735707 | ususa | | usgsu | | AUUU | |
| AD- | csasagugUfaUfGfUfguauaaa | 4695 | VPusAfscuuUfaUfAfcacaUfaCfacuu | 4155 | UACAAGUGUAUGUGUAUAA | 4424 |
| 1735708 | gsusa | | gsusa | | AGUC | |
| AD- | usascaagCfaUfGfCfauauuga | 4696 | VPusUfscucAfaUfAfugcaUfgCfuugu | 4156 | UGUACAAGCAUGCAUAUUG | 4425 |
| 1735709 | gsasa | | ascsa | | AGAU | |
| AD- | usgsugucUfuUfUfAfugucuuu | 4697 | VPusUfscaaAfgAfCfauaaAfaGfacac | 4157 | AGUGUGUCUUUUAUGUCUU | 4426 |
| 1735710 | gsasa | | ascsu | | UGAA | |
| AD- | asusuuucUfcGfCfAfgaauuca | 4698 | VPusAfsuugAfaUfUfcugcGfaGfaaaa | 4158 | UGAUUUUCUCGCAGAAUUC | 4427 |
| 1735711 | asusa | | uscsa | | AAUG | |
| AD- | usasgcgaCfaGfUfUfugaguaa | 4699 | VPusUfsuuuAfcUfCfaaacUfgUfcgcu | 4159 | AAUAGCGACAGUUUGAGUA | 4428 |
| 1735712 | asasa | | asusu | | AAAG | |
| AD- | csusggugUfaUfGfAfauacuuu | 4700 | VPusAfscaaAfgUfAfuucaUfaCfacca | 4160 | CACUGGUGUAUGAAUACUU | 4429 |
| 1735713 | gsusa | | gsusg | | UGUC | |
| AD- | gsusgauaAfgUfGfGfcuaacag | 4701 | VPusUfsucuGfuUfAfgccaCfuUfauca | 4161 | AGGUGAUAAGUGGCUAACA | 4430 |
| 1735714 | asasa | | cscsu | | GAAG | |
| AD- | gsusaggcUfaAfUfAfuuuucua | 4702 | VPusAfsauaGfaAfAfauauUfaGfccua | 4162 | UGGUAGGCUAAUAUUUUCU | 4431 |
| 1735715 | ususa | | cscsa | | AUUA | |
| AD- | cscsucuuCfaUfCfCfucgacug | 4703 | VPusGfsacaGfuCfGfaggaUfgAfagag | 4163 | CGCCUCUUCAUCCUCGACU | 4432 |
| 1735716 | uscsa | | gscsg | | GUCC | |
| AD- | asasuugcCfaUfAfAfgccauau | 4704 | VPusUfsaauAfuGfGfcuuaUfgGfcaau | 4164 | GAAAUUGCCAUAAGCCAUA | 4433 |
| 1735717 | usasa | | ususc | | UUAC | |
| AD- | gscsgaauAfuUfUfUfgucagcu | 4705 | VPusUfsuagCfuGfAfcaaaAfuAfuucg | 4165 | CGGCGAAUAUUUUGUCAGC | 4434 |
| 1735718 | asasa | | cscsg | | UAAU | |
| AD- | asusccagUfgUfUfGfccuuucu | 4706 | VPusUfscagAfaAfGfgcaaCfaCfugga | 4166 | GAAUCCAGUGUUGCCUUUC | 4435 |
| 1735719 | gsasa | | ususc | | UGAG | |
| AD- | asasagucUfaUfGfCfcugucua | 4707 | VPusUfsuuaGfaCfAfggcaUfaGfacuu | 4167 | GAAAAGUCUAUGCCUGUCU | 4436 |
| 1735720 | asasa | | ususc | | AAAA | |
| AD- | gsgsacauCfaGfAfAfguuugaa | 4708 | VPusAfsauuCfaAfAfcuucUfgAfuguc | 4168 | AUGGACAUCAGAAGUUUGA | 4437 |
| 1735721 | ususa | | csasu | | AUUC | |
| AD- | gsgscgaaUfuGfCfUfuauauuu | 4709 | VPusAfsgaaAfuAfUfaagcAfaUfucgc | 4169 | UGGGCGAAUUGCUUAUAUU | 4438 |
| 1735722 | csusa | | cscsa | | UCUU | |
| AD- | csascacuAfuGfUfGfuaaacca | 4710 | VPusAfscugGfuUfUfacacAfuAfgugu | 4170 | CACACACUAUGUGUAAACC | 4439 |
| 1735723 | gsusa | | gsusg | | AGUC | |
| AD- | csasggugAfaGfAfAfgaaaaca | 4711 | VPusAfsaugUfuUfUfcuucUfuCfaccu | 4171 | AUCAGGUGAAGAAGAAAAC | 4440 |
| 1735724 | ususa | | gsasu | | AUUU | |
| AD- | gsusgagcUfcUfUfAfuuuuuca | 4712 | VPusAfsgugAfaAfAfauaaGfaGfcuca | 4172 | UAGUGAGCUCUUAUUUUUC | 4441 |
| 1735725 | csusa | | csusa | | ACUG | |
| AD- | asasagcuGfaUfGfGfgaauaaa | 4713 | VPusAfsguuUfaUfUfcccaUfcAfgcuu | 4173 | GUAAAGCUGAUGGGAAUAA | 4442 |
| 1735726 | csusa | | usasc | | ACUG | |
| AD- | gsusgucgUfuAfGfGfagaaacu | 4714 | VPusGfscagUfuUfCfuccuAfaCfgaca | 4174 | GCGUGUCGUUAGGAGAAAC | 4443 |
| 1735727 | gscsa | | csgsc | | UGCA | |
| AD- | gsgsgacuUfaAfAfAfcaauggu | 4715 | VPusAfsuacCfaUfUfguuuUfaAfgucc | 4175 | UUGGGACUUAAAACAAUGG | 4444 |
| 1735728 | asusa | | csasa | | UAUA | |
| AD- | csusgaugGfuAfGfAfauuaacc | 4716 | VPusAfsuggUfuAfAfuucuAfcCfauca | 4176 | UUCUGAUGGUAGAAUUAAC | 4445 |
| 1735729 | asusa | | gsasa | | CAUG | |
| AD- | ascsuaugUfgUfGfUfuuuauuu | 4717 | VPusAfsgaaAfuAfAfaacaCfaCfauag | 4177 | CUACUAUGUGUGUUUUAUU | 4446 |
| 1735730 | csusa | | usasg | | UCUG | |
| AD- | csusgauaAfuUfUfAfuauuugc | 4718 | VPusGfsugcAfaAfUfauaaAfuUfauca | 4178 | GACUGAUAAUUUAUAUUUG | 4447 |
| 1735731 | ascsa | | gsusc | | CACU | |
| AD- | gsusccuuAfaAfUfUfauuuaac | 4719 | VPusGfsgguUfaAfAfuaauUfuAfagga | 4179 | UAGUCCUUAAAUUAUUUAA | 4448 |
| 1735732 | cscsa | | csusa | | CCCU | |
| AD- | csgscaauAfuGfGfUfgucagau | 4720 | VPusAfsgauCfuGfAfcaccAfuAfuugc | 4180 | CUCGCAAUAUGGUGUCAGA | 4449 |
| 1735733 | csusa | | gsasg | | UCUU | |
| AD- | ususcgaaGfaGfCfUfgcucuaa | 4721 | VPusGfsauuAfgAfGfcagcUfcUfucga | 4181 | UCUUCGAAGAGCUGCUCUA | 4450 |
| 1735734 | uscsa | | asgsa | | AUCC | |
| AD- | ususucugCfuGfAfGfaguauuc | 4722 | VPusCfsugaAfuAfCfucucAfgCfagaa | 4182 | UAUUUCUGCUGAGAGUAUU | 4451 |
| 1735735 | asgsa | | asusa | | CAGG | |
| AD- | ususgaacAfgGfAfAfguaucac | 4723 | VPusCfsaguGfaUfAfcuucCfuGfuuca | 4183 | UAUUGAACAGGAAGUAUCA | 4452 |
| 1735736 | usgsa | | asusa | | CUGG | |
| AD- | usgsccuuGfuAfUfUfuccagag | 4724 | VPusUfsucuCfuGfGfaaauAfcAfaggc | 4184 | GUUGCCUUGUAUUUCCAGA | 4453 |
| 1735737 | asasa | | asasc | | GAAC | |
| AD- | gsusuacuAfuGfGfCfaguugca | 4725 | VPusUfsuugCfaAfCfugccAfuAfguaa | 4185 | CAGUUACUAUGGCAGUUGC | 4454 |
| 1735738 | asasa | | csusg | | AAAA | |
| AD- | asusggcuGfaAfUfUfuuaaagc | 4726 | VPusCfsagcUfuUfAfaaauUfcAfgcca | 4186 | CCAUGGCUGAAUUUUAAAG | 4455 |
| 1735739 | usgsa | | usgsg | | CUGU | |
| AD- | gsusccauUfcAfUfGfgaucguu | 4727 | VPusAfsgaaCfgAfUfccauGfaAfugga | 4187 | CAGUCCAUUCAUGGAUCGU | 4456 |
| 1735740 | csusa | | csusg | | UCUU | |
| AD- | usgsuguaAfcUfCfCfuaugaug | 4728 | VPusAfsgcaUfcAfUfaggaGfuUfacac | 4188 | CCUGUGUAACUCCUAUGAU | 4457 |
| 1735741 | csusa | | asgsg | | GCUG | |
| AD- | usgsgcagUfuUfCfCfcacuccu | 4729 | VPusAfsuagGfaGfUfgggaAfaCfugcc | 4189 | GCUGGCAGUUUCCCACUCC | 4458 |
| 1735742 | asusa | | asgsc | | UAUG | |
| AD- | cscsucaaGfcAfGfAfugagauu | 4730 | VPusUfsgaaUfcUfCfaucuGfcUfugag | 4190 | UACCUCAAGCAGAUGAGAU | 4459 |
| 1735743 | csasa | | gsusa | | UCAG | |
| AD- | asusguugUfuUfUfCfucaaaag | 4731 | VPusUfsgcuUfuUfGfagaaAfaCfaaca | 4191 | UUAUGUUGUUUUCUCAAAA | 4460 |
| 1735744 | csasa | | usasa | | GCAG | |
| AD- | gsasgggaUfuUfCfUfgcucaau | 4732 | VPusGfscauUfgAfGfcagaAfaUfcccu | 4192 | CUGAGGGAUUUCUGCUCAA | 4461 |
| 1735745 | gscsa | | csasg | | UGCA | |
| AD- | gscscuucUfuCfUfUfccucacc | 4733 | VPusCfsaggUfgAfGfgaagAfaGfaagg | 4193 | GUGCCUUCUUCUUCCUCAC | 4462 |
| 1735746 | usgsa | | csasc | | CUGC | |
| AD- | ususgagaUfuGfAfAfucacauu | 4734 | VPusGfsaaaUfgUfGfauucAfaUfcuca | 4194 | UAUUGAGAUUGAAUCACAU | 4463 |
| 1735747 | uscsa | | asusa | | UUCC | |
| AD- | csusguucAfgUfGfCfuauucuc | 4735 | VPusGfsggaGfaAfUfagcaCfuGfaaca | 4195 | CACUGUUCAGUGCUAUUCU | 4464 |
| 1735748 | cscsa | | gsusg | | CCCA | |
| AD- | cscsuggcUfuAfCfAfgcagauc | 4736 | VPusUfsggaUfcUfGfcuguAfaGfccag | 4196 | UUCCUGGCUUACAGCAGAU | 4465 |
| 1735749 | csasa | | gsasa | | CCAC | |
| AD- | gsascagcAfgAfAfAfauuauuu | 4737 | VPusUfsgaaAfuAfAfuuuuCfuGfcugu | 4197 | AAGACAGCAGAAAAUUAUU | 4466 |
| 1735750 | csasa | | csusu | | UCAG | |
| AD- | ususggguUfuCfUfUfuaguuua | 4738 | VPusCfsauaAfaCfUfaaagAfaAfccca | 4198 | UAUUGGGUUUCUUUAGUUU | 4467 |
| 1735751 | usgsa | | asusa | | AUGU | |
| AD- | csasccauGfuAfGfGfucucaga | 4739 | VPusCfsuucUfgAfGfaccuAfcAfuggu | 4199 | CUCACCAUGUAGGUCUCAG | 4468 |
| 1735752 | asgsa | | gsasg | | AAGA | |
| AD- | gsusugugGfaAfCfAfggaaaug | 4740 | VPusUfsucaUfuUfCfcuguUfcCfacaa | 4200 | UGGUUGUGGAACAGGAAAU | 4469 |
| 1735753 | asasa | | cscsa | | GAAA | |
| AD- | gsgsagaaCfaUfAfUfcauauuu | 4741 | VPusCfsaaaAfuAfUfgauaUfgUfucuc | 4201 | UUGGAGAACAUAUCAUAUU | 4470 |
| 1735754 | usgsa | | csasa | | UUGG | |
| AD- | csasgccaAfaAfUfGfauggcaa | 4742 | VPusUfsuuuGfcCfAfucauUfuUfggcu | 4202 | AGCAGCCAAAAUGAUGGCA | 4471 |
| 1735755 | asasa | | gscsu | | AAAU | |
| AD- | cscsaauuUfuUfGfAfacuugua | 4743 | VPusUfscuaCfaAfGfuucaAfaAfauug | 4203 | UUCCAAUUUUUGAACUUGU | 4472 |
| 1735756 | gsasa | | gsasa | | AGAA | |
| AD- | asusaggaAfuAfGfUfuugccaa | 4744 | VPusAfsguuGfgCfAfaacuAfuUfccua | 4204 | AAAUAGGAAUAGUUUGCCA | 4473 |
| 1735757 | csusa | | ususu | | ACUC | |
| AD- | ususugaaUfgUfUfUfuggagaa | 4745 | VPusUfsuuuCfuCfCfaaaaCfaUfucaa | 4205 | UCUUUGAAUGUUUUGGAGA | 4474 |
| 1735758 | asasa | | asgsa | | AAAG | |
| AD- | gsuscaauAfuUfUfAfaugaauc | 4746 | VPusGfsugaUfuCfAfuuaaAfuAfuuga | 4206 | GUGUCAAUAUUUAAUGAAU | 4475 |
| 1735759 | ascsa | | csasc | | CACU | |
| AD- | usgsagaaUfaUfAfUfuaaaugc | 4747 | VPusGfsugcAfuUfUfaauaUfaUfucuc | 4207 | UGUGAGAAUAUAUUAAAUG | 4476 |
| 1735760 | ascsa | | ascsa | | CACA | |
| AD- | usasacaaGfgGfAfCfuaaauag | 4748 | VPusUfsccuAfuUfUfagucCfcUfuguu | 4208 | CUUAACAAGGGACUAAAUA | 4477 |
| 1735761 | gsasa | | asasg | | GGAA | |
| AD- | gsgscuucUfuCfUfUfccaccuc | 4749 | VPusCfsagaGfgUfGfgaagAfaGfaagc | 4209 | GCGGCUUCUUCUUCCACCU | 4478 |
| 1735762 | usgsa | | csgsc | | CUGC | |
| AD- | ususggaaCfaUfGfUfgguuauc | 4750 | VPusAfsagaUfaAfCfcacaUfgUfucca | 4210 | AAUUGGAACAUGUGGUUAU | 4479 |
| 1735763 | ususa | | asusu | | CUUU | |
| AD- | csasgagaAfcAfGfAfugauucu | 4751 | VPusAfsaagAfaUfCfaucuGfuUfcucu | 4211 | UCCAGAGAACAGAUGAUUC | 4480 |
| 1735764 | ususa | | gsgsa | | UUUG | |
| AD- | csusguuaUfuUfUfAfuuggguu | 4752 | VPusAfsaaaCfcCfAfauaaAfaUfaaca | 4212 | GUCUGUUAUUUUAUUGGGU | 4481 |
| 1735765 | ususa | | gsasc | | UUUA | |
| AD- | asusgaagAfaGfCfAfaaucuug | 4753 | VPusAfsccaAfgAfUfuugcUfuCfuuca | 4213 | AGAUGAAGAAGCAAAUCUU | 4482 |
| 1735766 | gsusa | | uscsu | | GGUC | |
| AD- | ususuggaAfuCfAfUfgcaauuu | 4754 | VPusCfsaaaAfuUfGfcaugAfuUfccaa | 4214 | AUUUUGGAAUCAUGCAAUU | 4483 |
| 1735767 | usgsa | | asasu | | UUGC | |
| AD- | csasauguUfuUfGfUfaaacuug | 4755 | VPusAfsgcaAfgUfUfuacaAfaAfcauu | 4215 | UACAAUGUUUUGUAAACUU | 4484 |
| 1735768 | csusa | | gsusa | | GCUA | |
| AD- | gsgsaucgUfaAfUfAfauggcaa | 4756 | VPusUfsuuuGfcCfAfuuauUfaCfgauc | 4216 | UGGGAUCGUAAUAAUGGCA | 4485 |
| 1735769 | asasa | | cscsa | | AAAG | |
| AD- | gsusgcuuUfcUfCfAfgguuccu | 4757 | VPusAfscagGfaAfCfcugaGfaAfagca | 4217 | GGGUGCUUUCUCAGGUUCC | 4486 |
| 1735770 | gsusa | | cscsc | | UGUA | |
| AD- | asusgauaCfuGfAfAfaguggug | 4758 | VPusAfsucaCfcAfCfuuucAfgUfauca | 4218 | UAAUGAUACUGAAAGUGGU | 4487 |
| 1735771 | asusa | | ususa | | GAUG | |
| AD- | uscsacugGfaCfCfUfgauuuua | 4759 | VPusAfscuaAfaAfUfcaggUfcCfagug | 4219 | UAUCACUGGACCUGAUUUU | 4488 |
| 1735772 | gsusa | | asusa | | AGUA | |
| AD- | gsasacuuUfuUfGfCfuuuuaug | 4760 | VPusUfsucaUfaAfAfagcaAfaAfaguu | 4220 | AGGAACUUUUUGCUUUUAU | 4489 |
| 1735773 | asasa | | cscsu | | GAAA | |
| AD- | gsasacacAfgCfAfCfagauuug | 4761 | VPusAfsacaAfaUfCfugugCfuGfuguu | 4221 | AUGAACACAGCACAGAUUU | 4490 |
| 1735774 | ususa | | csasu | | GUUA | |
| AD- | asusauggAfaGfGfAfaaucgua | 4762 | VPusUfscuaCfgAfUfuuccUfuCfcaua | 4222 | AGAUAUGGAAGGAAAUCGU | 4491 |
| 1735775 | gsasa | | uscsu | | AGAG | |
| AD- | gscsuaucUfaAfAfUfgggccuu | 4763 | VPusAfsaaaGfgCfCfcauuUfaGfauag | 4223 | AUGCUAUCUAAAUGGGCCU | 4492 |
| 1735776 | ususa | | csasu | | UUUA | |
| AD- | csasaaacCfaCfCfAfagaagga | 4764 | VPusUfsuucCfuUfCfuuggUfgGfuuuu | 4224 | UCCAAAACCACCAAGAAGG | 4493 |
| 1735777 | asasa | | gsgsa | | AAAA | |
| AD- | cscsacagAfuGfUfGfcuacaug | 4765 | VPusUfsgcaUfgUfAfgcacAfuCfugug | 4225 | UGCCACAGAUGUGCUACAU | 4494 |
| 1735778 | csasa | | gscsa | | GCAG | |
| AD- | asasagucAfuAfCfAfuguacaa | 4766 | VPusGfscuuGfuAfCfauguAfuGfacuu | 4226 | AUAAAGUCAUACAUGUACA | 4495 |
| 1735779 | gscsa | | usasu | | AGCA | |
| AD- | usasgaagCfaGfAfAfgaggaua | 4767 | VPusAfsuuaUfcCfUfcuucUfgCfuucu | 4227 | GUUAGAAGCAGAAGAGGAU | 4496 |
| 1735780 | asusa | | asasc | | AAUG | |
| AD- | csgsugugUfuCfGfCfacagcuu | 4768 | VPusUfscaaGfcUfGfugcgAfaCfacac | 4228 | AGCGUGUGUUCGCACAGCU | 4497 |
| 1735781 | gsasa | | gscsu | | UGAG | |
| AD- | gscsugguGfuUfUfUfguccag | 4769 | VPusAfsgacUfgGfAfcaaaAfcAfccag | 4229 | GAGCUGGUGUUUUGUCCAG | 4498 |
| 1735782 | ucsusa | | csusc | | UCUG | |
| AD- | asusggaaAfaCfAfAfucuaaau | 4770 | VPusGfsgauUfuAfGfauugUfuUfucca | 4230 | AAAUGGAAAACAAUCUAAA | 4499 |
| 1735783 | cscsa | | ususu | | UCCA | |
| AD- | csasuuagAfaAfAfUfggcuauc | 4771 | VPusUfscgaUfaGfCfcauuUfuCfuaau | 4231 | UGCAUUAGAAAAUGGCUAU | 4500 |
| 1735784 | gsasa | | gscsa | | CGAG | |
| AD- | asusggacAfgAfAfGfgcauugu | 4772 | VPusUfsgacAfaUfGfccuuCfuGfucca | 4232 | AAAUGGACAGAAGGCAUUG | 4501 |
| 1735785 | csasa | | ususu | | UCAA | |
| AD- | csasgauuGfcUfGfCfaguggaa | 4773 | VPusUfsuuuCfcAfCfugcaGfcAfaucu | 4233 | UACAGAUUGCUGCAGUGGA | 4502 |
| 1735786 | asasa | | gsusa | | AAAU | |
| AD- | asgsguggAfaAfUfGfgaguuga | 4774 | VPusGfsaucAfaCfUfccauUfuCfcacc | 4234 | GGAGGUGGAAAUGGAGUUG | 4503 |
| 1735787 | uscsa | | uscsc | | AUCU | |
| AD- | csusgggcAfaUfGfCfaccuaau | 4775 | VPusGfsuauUfaGfGfugcaUfuGfccca | 4235 | CUCUGGGCAAUGCACCUAA | 4504 |
| 1735788 | ascsa | | gsasg | | UACU | |
| AD- | asusuuccCfuCfUfGfccuaaag | 4776 | VPusGfsucuUfuAfGfgcagAfgGfgaaa | 4236 | UAAUUUCCCUCUGCCUAAA | 4505 |
| 1735789 | ascsa | | ususa | | GACU | |
| AD- | csgscuucUfgCfAfAfcguggag | 4777 | VPusAfsgcuCfcAfCfguugCfaGfaagc | 4237 | GCCGCUUCUGCAACGUGGA | 4506 |
| 1735790 | csusa | | gsgsc | | GCUG | |
| AD- | asgsauucAfuAfUfUfguauuuc | 4778 | VPusGfsggaAfaUfAfcaauAfuGfaauc | 4238 | AUAGAUUCAUAUUGUAUUU | 4507 |
| 1735791 | cscsa | | usasu | | CCCA | |
| AD- | asgsgacaAfaUfGfCfauuucua | 4779 | VPusAfscuaGfaAfAfugcaUfuUfgucc | 4239 | GGAGGACAAAUGCAUUUCU | 4508 |
| 1735792 | gsusa | | uscsc | | AGUG | |
| AD- | ususagacGfuGfUfUfgagugag | 4780 | VPusCfsacuCfaCfUfcaacAfcGfucua | 4240 | UAUUAGACGUGUUGAGUGA | 4509 |
| 1735793 | usgsa | | asusa | | GUGC | |
| AD- | csusguggAfcAfUfCfaaaugcu | 4781 | VPusUfscagCfaUfUfugauGfuCfcaca | 4241 | AGCUGUGGACAUCAAAUGC | 4510 |
| 1735794 | gsasa | | gscsu | | UGAA | |
| AD- | asgsgccuUfaAfUfAfcugugag | 4782 | VPusCfsucuCfaCfAfguauUfaAfggcc | 4242 | ACAGGCCUUAAUACUGUGA | 4511 |
| 1735795 | asgsa | | usgsu | | GAGG | |
| AD- | asgsaaauCfaUfUfGfccuagug | 4783 | VPusAfsacaCfuAfGfgcaaUfgAfuuuc | 4243 | GCAGAAAUCAUUGCCUAGU | 4512 |
| 1735796 | ususa | | usgsc | | GUUC | |
| AD- | usgsagugCfuGfAfGfuuccuug | 4784 | VPusAfsgcaAfgGfAfacucAfgCfacuc | 4244 | AGUGAGUGCUGAGUUCCUU | 4513 |
| 1735797 | csusa | | ascsu | | GCUG | |
| AD- | gscscaugCfaUfGfAfuuaugau | 4785 | VPusUfsgauCfaUfAfaucaUfgCfaugg | 4245 | CUGCCAUGCAUGAUUAUGA | 4514 |
| 1735798 | csasa | | csasg | | UCAC | |
| AD- | usgscugcUfgGfUfUfugcuacc | 4786 | VPusCfsuggUfaGfCfaaacCfaGfcagc | 4246 | GAUGCUGCUGGUUUGCUAC | 4515 |
| 1735799 | asgsa | | asusc | | CAGG | |
| AD- | csasgaugAfaGfAfGfgaauagc | 4787 | VPusUfscgcUfaUfUfccucUfuCfaucu | 4247 | GGCAGAUGAAGAGGAAUAG | 4516 |
| 1735800 | gsasa | | gscsc | | CGAC | |
| AD- | asusccagGfuGfCfAfucaauuu | 4788 | VPusAfsgaaAfuUfGfaugcAfcCfugga | 4248 | AAAUCCAGGUGCAUCAAUU | 4517 |
| 1735801 | csusa | | ususu | | UCUG | |
| AD- | asasuaagCfcAfAfGfcguuauc | 4789 | VPusAfsagaUfaAfCfgcuuGfgCfuuau | 4249 | UUAAUAAGCCAAGCGUUAU | 4518 |
| 1735802 | ususa | | usasa | | CUUG | |
| AD- | gsasgaggAfuCfCfUfugcucug | 4790 | VPusAfsgcaGfaGfCfaaggAfuCfcucu | 4250 | GUGAGAGGAUCCUUGCUCU | 4519 |
| 1735803 | csusa | | csasc | | GCUG | |
| AD- | asusgaugAfaGfAfCfggugaag | 4791 | VPusUfsucuUfcAfCfcgucUfuCfauca | 4251 | UGAUGAUGAAGACGGUGAA | 4520 |
| 1735804 | asasa | | uscsa | | GAAU | |
| AD- | uscsguaaGfuGfUfAfagaaaga | 4792 | VPusAfsuucUfuUfCfuuacAfcUfuacg | 4252 | AUUCGUAAGUGUAAGAAAG | 4521 |
| 1735805 | asusa | | asasu | | AAUG | |
| AD- | asuscuguGfuAfAfCfagcugug | 4793 | VPusUfsccaCfaGfCfuguuAfcAfcaga | 4253 | CAAUCUGUGUAACAGCUGU | 4522 |
| 1735806 | gsasa | | ususg | | GGAC | |
| AD- | asusuuaaAfaUfUfCfccacuca | 4794 | VPusGfsuugAfgUfGfggaaUfuUfuaaa | 4254 | AUAUUUAAAAUUCCCACUC | 4523 |
| 1735807 | ascsa | | usasu | | AACA | |
| AD- | gsasccagUfgUfCfUfacuggcu | 4795 | VPusAfsgagCfcAfGfuagaCfaCfuggu | 4255 | UGGACCAGUGUCUACUGGC | 4524 |
| 1735808 | csusa | | cscsa | | UCUC | |
| AD- | csgscuuuCfgUfUfUfuuuaguc | 4796 | VPusAfsugaCfuAfAfaaaaCfgAfaagc | 4256 | AGCGCUUUCGUUUUUUAGU | 4525 |
| 1735809 | asusa | | gscsu | | CAUU | |
| AD- | uscscgugUfuGfAfCfccauguu | 4797 | VPusGfscaaCfaUfGfggucAfaCfacgg | 4257 | CAUCCGUGUUGACCCAUGU | 4526 |
| 1735810 | gscsa | | asusg | | UGCA | |
| AD- | asusacagAfaGfAfUfgaagaaa | 4798 | VPusCfsauuUfcUfUfcaucUfuCfugua | 4258 | AGAUACAGAAGAUGAAGAA | 4527 |
| 1735811 | usgsa | | uscsu | | AUGG | |
| AD- | uscsucccGfaGfCfUfgaaacuu | 4799 | VPusUfsuaaGfuUfUfcagcUfcGfggag | 4259 | UCUCUCCCGAGCUGAAACU | 4528 |
| 1735812 | asasa | | asgsa | | UAAA | |
| AD- | usgscaucAfaAfCfUfggcagau | 4800 | VPusAfsuauCfuGfCfcaguUfuGfaugc | 4260 | UGUGCAUCAAACUGGCAGA | 4529 |
| 1735813 | asusa | | ascsa | | UAUA | |
| AD- | ususgcaaAfaUfAfUfucaggag | 4801 | VPusGfsucuCfcUfGfaauaUfuUfugca | 4261 | AGUUGCAAAAUAUUCAGGA | 4530 |
| 1735814 | ascsa | | ascsu | | GACC | |
| AD- | ascsaaucGfuAfUfAfcaugcca | 4802 | VPusUfsgugGfcAfUfguauAfcGfauug | 4262 | UCACAAUCGUAUACAUGCC | 4531 |
| 1735815 | csasa | | usgsa | | ACAG | |
| AD- | asasuagaAfaAfGfAfugagcaa | 4803 | VPusAfsguuGfcUfCfaucuUfuUfcuau | 4263 | UUAAUAGAAAAGAUGAGCA | 4532 |
| 1735816 | csusa | | usasa | | ACUG | |
| AD- | usasucuuAfuGfUfUfccauggc | 4804 | VPusCfsagcCfaUfGfgaacAfuAfagau | 4264 | CUUAUCUUAUGUUCCAUGG | 4533 |
| 1735817 | usgsa | | asasg | | CUGA | |
| AD- | csusacguGfaAfGfAfgcugogu | 4805 | VPusUfscacGfcAfGfcucuUfcAfcgua | 4265 | GGCUACGUGAAGAGCUGCG | 4534 |
| 1735818 | gsasa | | gscsc | | UGAG | |
| AD- | gsasauugAfuGfGfCfucuauac | 4806 | VPusAfscguAfuAfGfagccAfuCfaauu | 4266 | UAGAAUUGAUGGCUCUAUA | 4535 |
| 1735819 | gsusa | | csusa | | CGUG | |
| AD- | ususcaggUfaAfUfUfgaagagg | 4807 | VPusUfsgccUfcUfUfcaauUfaCfcuga | 4267 | GAUUCAGGUAAUUGAAGAG | 4536 |
| 1735820 | csasa | | asusc | | GCAG | |
| AD- | asasggacUfgAfGfUfgaccuuu | 4808 | VPusAfscaaAfgGfUfcacuCfaGfuccu | 4268 | UGAAGGACUGAGUGACCUU | 4537 |
| 1735821 | gsusa | | uscsa | | UGUU | |
| AD- | csgsagagUfcUfGfAfggaacgg | 4809 | VPusAfsgccGfuUfCfcucaGfaCfucuc | 4269 | CCCGAGAGUCUGAGGAACG | 4538 |
| 1735822 | csusa | | gsgsg | | GCUA | |
| AD- | asusucuuUfgGfGfAfuugggac | 4810 | VPusAfsaguCfcCfAfauccCfaAfagaa | 4270 | UGAUUCUUUGGGAUUGGGA | 4539 |
| 1735823 | ususa | | uscsa | | CUUA | |
| AD- | usasccagGfuCfAfGfugguuag | 4811 | VPusUfsucuAfaCfCfacugAfcCfuggu | 4271 | GCUACCAGGUCAGUGGUUA | 4540 |
| 1735824 | asasa | | asgsc | | GAAG | |
| AD- | asusucagGfuUfUfGfccaugga | 4812 | VPusUfsgucCfaUfGfgcaaAfcCfugaa | 4272 | GUAUUCAGGUUUGCCAUGG | 4541 |
| 1735825 | csasa | | usasc | | ACAU | |
| AD- | gscsagcuAfaAfCfUfgguccug | 4813 | VPusUfsccaGfgAfCfcaguUfuAfgcug | 4273 | CGGCAGCUAAACUGGUCCU | 4542 |
| 1735826 | gsasa | | cscsg | | GGAG | |
| AD- | asgsuaauGfgCfAfUfagaaugg | 4814 | VPusCfsuccAfuUfCfuaugCfcAfuuac | 4274 | AUAGUAAUGGCAUAGAAUG | 4543 |
| 1735827 | asgsa | | usasu | | GAGU | |
| AD- | asasuugcAfaGfAfAfaauggac | 4815 | VPusUfsgguCfcAfUfuuucUfuGfcaau | 4275 | AUAAUUGCAAGAAAAUGGA | 4544 |
| 1735828 | csasa | | usasu | | CCAU | |
| AD- | csgsgcacUfgCfGfUfucuggug | 4816 | VPusAfsgcaCfcAfGfaacgCfaGfugcc | 4276 | UCCGGCACUGCGUUCUGGU | 4545 |
| 1735829 | csusa | | gsgsa | | GCUG | |
| AD- | asgsucauAfuUfGfAfagaagcc | 4817 | VPusUfsgggCfuUfCfuucaAfuAfugac | 4277 | AAAGUCAUAUUGAAGAAGC | 4546 |
| 1735830 | csasa | | ususu | | CCAG | |
| AD- | uscsaucaUfgCfUfGfcgucagc | 4818 | VPusAfsagcUfgAfCfgcagCfaUfgaug | 4278 | AAUCAUCAUGCUGCGUCAG | 4547 |
| 1735831 | ususa | | asusu | | CUUG | |
| AD- | asasaugaUfcGfCfCfucuuggu | 4819 | VPusAfsuacCfaAfGfaggcGfaUfcauu | 4279 | GAAAAUGAUCGCCUCUUGG | 4548 |
| 1735832 | asusa | | ususc | | UAUG | |
| AD- | asusuugaAfgAfUfAfuaaagag | 4820 | VPusUfsgcuCfuUfUfauauCfuUfcaaa | 4280 | ACAUUUGAAGAUAUAAAGA | 4549 |
| 1735833 | csasa | | usgsu | | GCAG | |
| AD- | gsgsucucCfuUfCfAfccagccu | 4821 | VPusCfsgagGfcUfGfgugaAfgGfagac | 4281 | UGGGUCUCCUUCACCAGCC | 4550 |
| 1735834 | csgsa | | cscsa | | UCGG | |
| AD- | gsasaacaGfaGfAfAfgaaagac | 4822 | VPusCfsuguCfuUfUfcuucUfcUfguuu | 4282 | AGGAAACAGAGAAGAAAGA | 4551 |
| 1735835 | asgsa | | cscsu | | CAGC | |
| AD- | ususcuagUfgGfCfUfacaaaug | 4823 | VPusGfsgcaUfuUfGfuagcCfaCfuaga | 4283 | AUUUCUAGUGGCUACAAAU | 4552 |
| 1735836 | cscsa | | asasu | | GCCC | |
| AD- | gsgsagagAfaAfUfCfaggaagg | 4824 | VPusAfsuccUfuCfCfugauUfuCfucuc | 4284 | UGGGAGAGAAAUCAGGAAG | 4553 |
| 1735837 | asusa | | cscsa | | GAUU | |
| AD- | asusauagUfgAfGfCfccuaaag | 4825 | VPusUfsccuUfuAfGfggcuCfaCfuaua | 4285 | AAAUAUAGUGAGCCCUAAA | 4554 |
| 1735838 | gsasa | | ususu | | GGAC | |
| AD- | gscsugccUfuUfGfUfccucgcc | 4826 | VPusAfsgggCfgAfGfgacaAfaGfgcag | 4286 | UGGCUGCCUUUGUCCUCGC | 4555 |
| 1735839 | csusa | | cscsa | | CCUG | |
| AD- | gsgscaaaAfgAfCfCfucaccaa | 4827 | VPusUfscuuGfgUfGfagguCfuUfuugc | 4287 | AUGGCAAAAGACCUCACCA | 4556 |
| 1735840 | gsasa | | csasu | | AGAA | |
| AD- | ususcagcAfuCfGfCfcugugcc | 4828 | VPusAfsaggCfaCfAfggcgAfuGfcuga | 4288 | UCUUCAGCAUCGCCUGUGC | 4557 |
| 1735841 | ususa | | asgsa | | CUUC | |
| AD- | gscsuaaaUfgAfGfGfcucgcaa | 4829 | VPusUfsauuGfcGfAfgccuCfaUfuuag | 4289 | GUGCUAAAUGAGGCUCGCA | 4558 |
| 1735842 | usasa | | csasc | | AUAU | |
| AD- | csasgcuuCfgUfCfUfggugggu | 4830 | VPusAfsgacCfcAfCfcagaCfgAfagcu | 4290 | GCCAGCUUCGUCUGGUGGG | 4559 |
| 1735843 | csusa | | gsgsc | | UCUC | |
| AD- | asusauggUfaUfUfGfgcaugca | 4831 | VPusAfscugCfaUfGfccaaUfaCfcaua | 4291 | GAAUAUGGUAUUGGCAUGC | 4560 |
| 1735844 | gsusa | | ususc | | AGUU | |
| AD- | asasacugCfaGfGfUfggagaau | 4832 | VPusGfsaauUfcUfCfcaccUfgCfaguu | 4292 | AUAAACUGCAGGUGGAGAA | 4561 |
| 1735845 | uscsa | | usasu | | UUCC | |
| AD- | usasuggcUfgCfCfUfgucuuca | 4833 | VPusUfsuugAfaGfAfcaggCfaGfccau | 4293 | GUUAUGGCUGCCUGUCUUC | 4562 |
| 1735846 | asasa | | asasc | | AAAC | |
| AD- | csusguucUfgAfAfAfuagagga | 4834 | VPusGfsgucCfuCfUfauuuCfaGfaaca | 4294 | CCCUGUUCUGAAAUAGAGG | 4563 |
| 1735847 | cscsa | | gsgsg | | ACCC | |
| AD- | uscsuauuUfuUfAfAfagccgcu | 4835 | VPusCfscagCfgGfCfuuuaAfaAfauag | 4295 | UUUCUAUUUUUAAAGCCGC | 4564 |
| 1735848 | gsgsa | | asasa | | UGGU | |
| AD- | gscsugagGfaGfAfAfgcucagg | 4836 | VPusUfsuccUfgAfGfcuucUfcCfucag | 4296 | CAGCUGAGGAGAAGCUCAG | 4565 |
| 1735849 | asasa | | csusg | | GAAC | |
| AD- | csasagccUfuCfCfAfccacaag | 4837 | VPusGfsacuUfgUfGfguggAfaGfgcuu | 4297 | UCCAAGCCUUCCACCACAA | 4566 |
| 1735850 | uscsa | | gsgsa | | GUCA | |
| AD- | ususcaguUfaCfCfAfaaguggc | 4838 | VPusUfsggcCfaCfUfuuggUfaAfcuga | 4298 | AUUUCAGUUACCAAAGUGG | 4567 |
| 1735851 | csasa | | asasu | | CCAG | |
| AD- | csasccugCfuUfCfCfucacccg | 4839 | VPusUfsccgGfgUfGfaggaAfgCfaggu | 4299 | CUCACCUGCUUCCUCACCC | 4568 |
| 1735852 | gsasa | | gsasg | | GGAC | |
| AD- | ususuuauCfaCfCfCfacauagu | 4840 | VPusCfscacUfaUfGfugggUfgAfuaaa | 4300 | UCUUUUAUCACCCACAUAG | 4569 |
| 1735853 | gsgsa | | asgsa | | UGGG | |
| AD- | asgsgagaCfcGfUfCfguugccu | 4841 | VPusCfsaagGfcAfAfcgacGfgUfcucc | 4301 | UCAGGAGACCGUCGUUGCC | 4570 |
| 1735854 | usgsa | | usgsa | | UUGU | |
| AD- | asusggccCfaGfCfAfaaaguuc | 4842 | VPusUfscgaAfcUfUfuugcUfgGfgcca | 4302 | AAAUGGCCCAGCAAAAGUU | 4571 |
| 1735855 | gsasa | | ususu | | CGAG | |
| AD- | asgsacgcCfaAfAfGfccaugcg | 4843 | VPusAfsccgCfaUfGfgcuuUfgGfcguc | 4303 | CGAGACGCCAAAGCCAUGC | 4572 |
| 1735856 | gsusa | | uscsg | | GGUC | |
| AD- | csasgcuaAfuGfCfAfccaccuc | 4844 | VPusGfsugaGfgUfGfgugcAfuUfagcu | 4304 | GUCAGCUAAUGCACCACCU | 4573 |
| 1735857 | ascsa | | gsasc | | CACU | |
| AD- | gsgsaaaaGfcAfGfAfcggcgaa | 4845 | VPusUfsauuCfgCfCfgucuGfcUfuuuc | 4305 | AAGGAAAAGCAGACGGCGA | 4574 |
| 1735858 | usasa | | csusu | | AUAU | |
| AD- | gscsuuugCfuGfGfGfuagugag | 4846 | VPusAfsgcuCfaCfUfacccAfgCfaaag | 4306 | CUGCUUUGCUGGGUAGUGA | 4575 |
| 1735859 | csusa | | csasg | | GCUC | |
| AD- | asasaugaAfaCfAfGfauucuga | 4847 | VPusCfsaucAfgAfAfucugUfuUfcauu | 4848 | GGAAAUGAAACAGAUUCUG | 4849 |
| 1735860 | usgsa | | uscsc | | AUGG | |

TABLE 16
Unmodified Sense and Antisense Strand Sequences of INHBC dsRNA Agents Comprising a GalNAc
Derivative Targeting Ligand

| | | SEQ | Range in | | SEQ | Range in |
|---|---|---|---|---|---|---|
| Duplex | Sense Sequence | ID | NM_ | Antisense Sequence | ID | NM_ |
| Name | 5′ to 3′ | NO: | 005538.4 | 5′ to 3 | NO: | 005538.4 |
| AD-1730493 | UCUUCCAGGGCCUCUGGCA | 4850 | 6-26 | ACUGCCAGAGGCCCUGGAAG | 4985 | 4-26 |
| | GU | | | AAG | | |
| AD-1730512 | CAGGACAGAGUUGAGACCA | 4851 | 27-47 | AGUGGUCUCAACUCUGUCCU | 4986 | 25-47 |
| | CU | | | GGC | | |
| AD-1730529 | CACAGCUGUUGAGACCCUG | 4852 | 44-64 | AUCAGGGUCUCAACAGCUGU | 4987 | 42-64 |
| | AU | | | GGU | | |
| AD-1730554 | GAGUCUGUAUUGCUCAAGA | 4853 | 69-89 | AUUCUUGAGCAAUACAGACU | 4988 | 67-89 |
| | AU | | | CAG | | |
| AD-1730570 | UGACCUCCUCAUUGCUUCU | 4854 | 105-125 | ACAGAAGCAAUGAGGAGGUC | 4989 | 103-125 |
| | GU | | | AUU | | |
| AD-1730588 | UGGCCUUUCUCCUCCUGGC | 4855 | 123-143 | AAGCCAGGAGGAGAAAGGCC | 4990 | 121-143 |
| | UU | | | AGA | | |
| AD-1730606 | CUCCAACCACAGUGGCCAC | 4856 | 141-161 | AAGUGGCCACUGUGGUUGGA | 4991 | 139-161 |
| | UU | | | GCC | | |
| AD-1730636 | GCGGUCAGUGUCCAGCAUG | 4857 | 171-191 | AACAUGCUGGACACUGACCG | 4992 | 169-191 |
| | UU | | | CCA | | |
| AD-1730643 | ACCUUGGAACUGGAGAGCC | 4858 | 200-220 | AUGGCUCUCCAGUUCCAAGG | 4993 | 198-220 |
| | AU | | | UGG | | |
| AD-1730667 | GAGCUGCUUCUUGAUCUGG | 4859 | 224-244 | AGCCAGAUCAAGAAGCAGCU | 4994 | 222-244 |
| | CU | | | CCC | | |
| AD-1730691 | AGAAGCAUCUUGGACAAGC | 4860 | 248-268 | AAGCUUGUCCAAGAUGCUUC | 4995 | 246-268 |
| | UU | | | UCU | | |
| AD-1730726 | CCAACACUGAACCGCCCUG | 4861 | 284-304 | AACAGGGCGGUUCAGUGUUG | 4996 | 282-304 |
| | UU | | | GGC | | |
| AD-1730749 | CAGAGCUGCUUUGAGGACU | 4862 | 307-327 | ACAGUCCUCAAAGCAGCUCU | 4997 | 305-327 |
| | GU | | | GGA | | |
| AD-1730765 | ACUGCACUGCAGCACCUCC | 4863 | 323-343 | AUGGAGGUGCUGCAGUGCAG | 4998 | 321-343 |
| | AU | | | UCC | | |
| AD-1730769 | GCACUUCUAGAGGACAACA | 4864 | 359-379 | ACUGUUGUCCUCUAGAAGUG | 4999 | 357-379 |
| | GU | | | CCC | | |
| AD-1730790 | GAACAGGAAUGUGAAAUCA | 4865 | 380-400 | AAUGAUUUCACAUUCCUGUU | 5000 | 378-400 |
| | UU | | | CCC | | |
| AD-1730807 | CAUCAGCUUUGCUGAGACA | 4866 | 397-417 | ACUGUCUCAGCAAAGCUGAU | 5001 | 395-417 |
| | GU | | | GAU | | |
| AD-1730825 | AGGCCUCUCCACCAUCAACC | 4867 | 415-435 | AGGUUGAUGGUGGAGAGGCC | 5002 | 413-435 |
| | U | | | UGU | | |
| AD-1730841 | AACCAGACUCGUCUUGAUU | 4868 | 431-451 | AAAAUCAAGACGAGUCUGGU | 5003 | 429-451 |
| | UU | | | UGA | | |
| AD-1730857 | AUUUUCACUUCUCCUCUGA | 4869 | 447-467 | AAUCAGAGGAGAAGUGAAA | 5004 | 445-467 |
| | UU | | | AUCA | | |
| AD-1730873 | UGAUAGAACUGCUGGUGAC | 4870 | 463-483 | AUGUCACCAGCAGUUCUAUC | 5005 | 461-483 |
| | AU | | | AGA | | |
| AD-1730910 | AGUCUCAUGUUCUUUGUGC | 4871 | 500-520 | AUGCACAAAGAACAUGAGAC | 5006 | 498-520 |
| | AU | | | UGG | | |
| AD-1730926 | UGCAGCUCCCUUCCAAUAC | 4872 | 516-536 | AGGUAUUGGAAGGGAGCUGC | 5007 | 514-536 |
| | CU | | | ACA | | |
| AD-1730942 | UACCACUUGGACCUUGAAA | 4873 | 532-552 | ACUUUCAAGGUCCAAGUGGU | 5008 | 530-552 |
| | GU | | | AUU | | |
| AD-1730958 | AAAGUGAGAGUCCUUGUGC | 4874 | 548-568 | AAGCACAAGGACUCUCACUU | 5009 | 546-568 |
| | UU | | | UCA | | |
| AD-1730982 | CCACAUAAUACCAACCUCA | 4875 | 572-592 | AGUGAGGUUGGUAUUAUGU | 5010 | 570-592 |
| | CU | | | GGAC | | |
| AD-1731003 | UUGGCUACUCAGUACCUGC | 4876 | 593-613 | AAGCAGGUACUGAGUAGCCA | 5011 | 591-613 |
| | UU | | | AGG | | |
| AD-1731019 | UGCUGGAGGUGGAUGCCAG | 4877 | 609-629 | AACUGGCAUCCACCUCCAGC | 5012 | 607-629 |
| | UU | | | AGG | | |
| AD-1731035 | CAGUGGCUGGCAUCAACUC | 4878 | 625-645 | AGGAGUUGAUGCCAGCCACU | 5013 | 623-645 |
| | CU | | | GGC | | |
| AD-1731042 | CCUGAAGCUCAAGCUGCCU | 4879 | 653-673 | ACAGGCAGCUUGAGCUUCAG | 5014 | 651-673 |
| | GU | | | GCC | | |
| AD-1731064 | GAGCUGGUACUUGAAGGCC | 4880 | 695-715 | AUGGCCUUCAAGUACCAGCU | 5015 | 693-715 |
| | AU | | | CCA | | |
| AD-1731088 | GCCCAGAGCUCAGUCAUCC | 4881 | 719-739 | AAGGAUGACUGAGCUCUGGG | 5016 | 717-739 |
| | UU | | | CUA | | |
| AD-1731117 | UGCCCAUAGGCCUUUUGUG | 4882 | 748-768 | ACCACAAAAGGCCUAUGGGC | 5017 | 746-768 |
| | GU | | | AGC | | |
| AD-1731134 | UGGCAGCCCGGGUGAGAGU | 4883 | 765-785 | AAACUCUCACCCGGGCUGCC | 5018 | 763-785 |
| | UU | | | ACA | | |
| AD-1731137 | GCAAACACCAGAUUCACCG | 4884 | 789-809 | AUCGGUGAAUCUGGUGUUUG | 5019 | 787-809 |
| | AU | | | CCC | | |
| AD-1731156 | ACGAGGCAUCGACUGCCAA | 4885 | 808-828 | ACUUGGCAGUCGAUGCCUCG | 5020 | 806-828 |
| | GU | | | UCG | | |
| AD-1731173 | AAGGAGGGUCCAGGAUGUG | 4886 | 825-845 | AGCACAUCCUGGACCCUCCU | 5021 | 823-845 |
| | CU | | | UGG | | |
| AD-1731189 | GUGCUGUCGACAAGAGUUU | 4887 | 841-861 | AAAAACUCUUGUCGACAGCA | 5022 | 839-861 |
| | UU | | | CAU | | |
| AD-1731205 | UUUUUUGUGGACUUCCGUG | 4888 | 857-877 | AUCACGGAAGUCCACAAAAA | 5023 | 855-877 |
| | AU | | | ACU | | |
| AD-1731234 | GCACGACUGGAUCAUCCAG | 4889 | 886-906 | AGCUGGAUGAUCCAGUCGUG | 5024 | 884-906 |
| | CU | | | CCA | | |
| AD-1731264 | CGCCAUGAACUUCUGCAUA | 4890 | 916-936 | ACUAUGCAGAAGUUCAUGGC | 5025 | 914-936 |
| | GU | | | GUA | | |
| AD-1731289 | UGCCCACUACACAUAGCAG | 4891 | 941-961 | ACCUGCUAUGUGUAGUGGGC | 5026 | 939-961 |
| | GU | | | ACU | | |
| AD-1731307 | GGCAUGCCUGGUAUUGCUG | 4892 | 959-979 | AGCAGCAAUACCAGGCAUGC | 5027 | 957-979 |
| | CU | | | CUG | | |
| AD-1731323 | CUGCCUCCUUUCACACUGC | 4893 | 975-995 | AUGCAGUGUGAAAGGAGGCA | 5028 | 973-995 |
| | AU | | | GCA | | |
| AD-1731340 | GCAGUGCUCAAUCUUCUCA | 4894 | 992-1012 | AUUGAGAAGAUUGAGCACUG | 5029 | 990-1012 |
| | AU | | | CAG | | |
| AD-1731358 | AAGGCCAACACAGCUGCAG | 4895 | 1010-1030 | ACCUGCAGCUGUGUUGGCCU | 5030 | 1008-1030 |
| | GU | | | UGA | | |
| AD-1731371 | GCUCAUGCUGUGUACCCAC | 4896 | 1044-1064 | ACGUGGGUACACAGCAUGAG | 5031 | 1042-1064 |
| | GU | | | CCC | | |
| AD-1731380 | GUCUCUGCUCUAUUAUGAC | 4897 | 1078-1098 | AUGUCAUAAUAGAGCAGAGA | 5032 | 1076-1098 |
| | AU | | | CAG | | |
| AD-1731398 | GACAGGGACAGCAACAUUG | 4898 | 1094-1114 | AACAAUGUUGCUGUCCCUGU | 5033 | 1092-1114 |
| | UU | | | CAU | | |
| AD-1731415 | UGUCAAGACUGACAUACCU | 4899 | 1111-1131 | ACAGGUAUGUCAGUCUUGAC | 5034 | 1109-1131 |
| | GU | | | AAU | | |
| AD-1731431 | CCUGACAUGGUAGUAGAGG | 4900 | 1127-1147 | AGCCUCUACUACCAUGUCAG | 5035 | 1125-1147 |
| | CU | | | GUA | | |
| AD-1731457 | AGUUAGUCUAUGUGUGGUA | 4901 | 1157-1177 | AAUACCACACAUAGACUAAC | 5036 | 1155-1177 |
| | UU | | | UGC | | |
| AD-1731484 | CCAAGGUUGCAUGGGAAAA | 4902 | 1184-1204 | AGUUUUCCCAUGCAACCUUG | 5037 | 1182-1204 |
| | CU | | | GGC | | |
| AD-1731492 | ACAGAAGUGCACUUCCUUG | 4903 | 1212-1232 | AUCAAGGAAGUGCACUUCUG | 5038 | 1210-1232 |
| | AU | | | UAG | | |
| AD-1731511 | AGAGGAGGGAAUGACCUCA | 4904 | 1231-1251 | AAUGAGGUCAUUCCCUCCUC | 5039 | 1229-1251 |
| | UU | | | UCA | | |
| AD-1731527 | UCAUUCUCUGUCCAGAAUG | 4905 | 1247-1267 | AACAUUCUGGACAGAGAAUG | 5040 | 1245-1267 |
| | UU | | | AGG | | |
| AD-1731545 | GUGGACUCCCUCUUCCUGA | 4906 | 1265-1285 | ACUCAGGAAGAGGGAGUCCA | 5041 | 1263-1285 |
| | GU | | | CAU | | |
| AD-1731562 | GAGCAUCUUAUGGAAAUUA | 4907 | 1282-1302 | AGUAAUUUCCAUAAGAUGCU | 5042 | 1280-1302 |
| | CU | | | CAG | | |
| AD-1731564 | CACCUUUGACUUGAAGAAA | 4908 | 1304-1324 | AGUUUCUUCAAGUCAAAGGU | 5043 | 1302-1324 |
| | CU | | | GGG | | |
| AD-1731584 | CUUCAUCUAAAGCAAGUCA | 4909 | 1324-1344 | AGUGACUUGCUUUAGAUGAA | 5044 | 1322-1344 |
| | CU | | | GGU | | |
| AD-1731600 | UCACUGUGCCAUCUUCCUG | 4910 | 1340-1360 | AUCAGGAAGAUGGCACAGUG | 5045 | 1338-1360 |
| | AU | | | ACU | | |
| AD-1731618 | GACCACUACCCUCUUUCCU | 4911 | 1358-1378 | AUAGGAAAGAGGGUAGUGG | 5046 | 1356-1378 |
| | AU | | | UCAG | | |
| AD-1731646 | UCCAUCCCGCUAGUCCAUCC | 4912 | 1386-1406 | AGGAUGGACUAGCGGGAUGG | 5047 | 1384-1406 |
| | U | | | ACU | | |
| AD-1731671 | ACCCAUCUCCAACCAUGAG | 4913 | 1431-1451 | AGCUCAUGGUUGGAGAUGGG | 5048 | 1429-1451 |
| | CU | | | UCU | | |
| AD-1731687 | GAGCAAUGCCAUCUGGUUC | 4914 | 1447-1467 | AGGAACCAGAUGGCAUUGCU | 5049 | 1445-1467 |
| | CU | | | CAU | | |
| AD-1731704 | UCCCAGGCAAAGACACCCU | 4915 | 1464-1484 | AAAGGGUGUCUUUGCCUGGG | 5050 | 1462-1484 |
| | UU | | | AAC | | |
| AD-1731720 | CCUUAGCUCACCUUUAAUA | 4916 | 1480-1500 | ACUAUUAAAGGUGAGCUAAG | 5051 | 1478-1500 |
| | GU | | | GGU | | |
| AD-1731728 | ACCCACUAUGCCUUCCUGU | 4917 | 1508-1528 | AGACAGGAAGGCAUAGUGGG | 5052 | 1506-1528 |
| | CU | | | UUA | | |
| AD-1731745 | GUCCUUUCUACUCAAUGGU | 4918 | 1525-1545 | AGACCAUUGAGUAGAAAGGA | 5053 | 1523-1545 |
| | CU | | | CAG | | |
| AD-1731750 | UCCAAGAUGAGUUGACACA | 4919 | 1550-1570 | AUUGUGUCAACUCAUCUUGG | 5054 | 1548-1570 |
| | AU | | | AGU | | |
| AD-1731753 | CAAUUUUUGUGGAUCUCCA | 4920 | 1580-1600 | ACUGGAGAUCCACAAAAAUU | 5055 | 1578-1600 |
| | GU | | | GGG | | |
| AD-1731782 | UCUUUGGAUUCACCAAAGU | 4921 | 1609-1629 | AAACUUUGGUGAAUCCAAAG | 5056 | 1607-1629 |
| | UU | | | AAG | | |
| AD-1731813 | UGCCCAAAAUAGAGGCUUA | 4922 | 1640-1660 | AGUAAGCCUCUAUUUUGGGC | 5057 | 1638-1660 |
| | CU | | | AGC | | |
| AD-1731819 | CUGUCCUUCUUAGUUGUCC | 4923 | 1685-1705 | AUGGACAACUAAGAAGGACA | 5058 | 1683-1705 |
| | AU | | | GGG | | |
| AD-1731842 | GAACUACUAAAGCUCUCUU | 4924 | 1708-1728 | AAAAGAGAGCUUUAGUAGU | 5059 | 1706-1728 |
| | UU | | | UCAC | | |
| AD-1731858 | CUUUGCAUACCUUCAUCCA | 4925 | 1724-1744 | AAUGGAUGAAGGUAUGCAA | 5060 | 1722-1744 |
| | UU | | | AGAG | | |
| AD-1731874 | CCAUUUUUUGUCCUUCUCU | 4926 | 1740-1760 | ACAGAGAAGGACAAAAAAUG | 5061 | 1738-1760 |
| | GU | | | GAU | | |
| AD-1731890 | GCCUUUCUCUAUGCCCUUA | 4927 | 1759-1779 | AUUAAGGGCAUAGAGAAAG | 5062 | 1757-1779 |
| | AU | | | GCAG | | |
| AD-1731906 | UUAAGGGCUGACUUGCCUG | 4928 | 1775-1795 | AUCAGGCAAGUCAGCCCUUA | 5063 | 1773-1795 |
| | AU | | | AGG | | |
| AD-1731923 | UGAGCUCUAUCACCUGAGC | 4929 | 1792-1812 | AAGCUCAGGUGAUAGAGCUC | 5064 | 1790-1812 |
| | UU | | | AGG | | |
| AD-1731932 | UCUGGCUUCCUGCUGAGGU | 4930 | 1821-1841 | AGACCUCAGCAGGAAGCCAG | 5065 | 1819-1841 |
| | CU | | | AGG | | |
| AD-1731958 | UUUCUUAUCCCUGUUCCCU | 4931 | 1847-1867 | AGAGGGAACAGGGAUAAGA | 5066 | 1845-1867 |
| | CU | | | AAUG | | |
| AD-1731978 | UCUGUCUAGGUGUCAUGGU | 4932 | 1867-1887 | AAACCAUGACACCUAGACAG | 5067 | 1865-1887 |
| | UU | | | AGA | | |
| AD-1731997 | UCUGUGUAACUGUGGCUAU | 4933 | 1886-1906 | AAAUAGCCACAGUUACACAG | 5068 | 1884-1906 |
| | UU | | | AAC | | |
| AD-1732013 | UAUUCUGUGUCCCUACACU | 4934 | 1902-1922 | AUAGUGUAGGGACACAGAAU | 5069 | 1900-1922 |
| | AU | | | AGC | | |
| AD-1732031 | GCCUACAUUCUGAUAUAAC | 4935 | 1953-1973 | AAGUUAUAUCAGAAUGUAG | 5070 | 1951-1973 |
| | UU | | | GCAG | | |
| AD-1732048 | CCUAAAGGCUUUCUAUCUU | 4936 | 1991-2011 | ACAAGAUAGAAAGCCUUUAG | 5071 | 1989-2011 |
| | GU | | | GAC | | |
| AD-1732062 | CUCAACAUCUCAUACUGGU | 4937 | 2025-2045 | AAACCAGUAUGAGAUGUUGA | 5072 | 2023-2045 |
| | UU | | | GGC | | |
| AD-1732078 | GGUUCCCUUAACUCUGCCU | 4938 | 2041-2061 | AUAGGCAGAGUUAAGGGAAC | 5073 | 2039-2061 |
| | AU | | | CAG | | |
| AD-1732094 | CCUAUACCUCUGUAAAUAA | 4939 | 2057-2077 | AAUUAUUUACAGAGGUAUA | 5074 | 2055-2077 |
| | UU | | | GGCA | | |
| AD-1732110 | UAAUUCCUUCACUAAGUUC | 4940 | 2073-2093 | AAGAACUUAGUGAAGGAAU | 5075 | 2071-2093 |
| | UU | | | UAUU | | |
| AD-1732157 | AAAGUCCUCUAUCUCCUAC | 4941 | 2120-2140 | AUGUAGGAGAUAGAGGACU | 5076 | 2118-2140 |
| | AU | | | UUUC | | |
| AD-1732173 | UACAAGGGCCCUAACUGGC | 4942 | 2136-2156 | AUGCCAGUUAGGGCCCUUGU | 5077 | 2134-2156 |
| | AU | | | AGG | | |
| AD-1732176 | CAGAUGACACAGAGCCUGC | 4943 | 2159-2179 | AGGCAGGCUCUGUGUCAUCU | 5078 | 2157-2179 |
| | CU | | | GGG | | |
| AD-1732195 | CUGCUUAUGCUGUAGUCUG | 4944 | 2178-2198 | AGCAGACUACAGCAUAAGCA | 5079 | 2176-2198 |
| | CU | | | GGC | | |
| AD-1732217 | ACUCUGCUGUCUCUUCACA | 4945 | 2200-2220 | AAUGUGAAGAGACAGCAGAG | 5080 | 2198-2220 |
| | UU | | | UAG | | |
| AD-1732235 | AUGGUCUCCUCAGAACUGA | 4946 | 2218-2238 | AUUCAGUUCUGAGGAGACCA | 5081 | 2216-2238 |
| | AU | | | UGU | | |
| AD-1732253 | AACUAUUGUAUCCAUCUCA | 4947 | 2236-2256 | AGUGAGAUGGAUACAAUAG | 5082 | 2234-2256 |
| | CU | | | UUCA | | |
| AD-1732269 | UCACACUUUAUGCCUCUUC | 4948 | 2252-2272 | AAGAAGAGGCAUAAAGUGU | 5083 | 2250-2272 |
| | UU | | | GAGA | | |
| AD-1732291 | AUCCUUCCAGAACCAUCUU | 4949 | 2294-2314 | AAAAGAUGGUUCUGGAAGG | 5084 | 2292-2314 |
| | UU | | | AUGG | | |
| AD-1732308 | UUUGAGGUCUCAUGGCUAA | 4950 | 2311-2331 | AAUUAGCCAUGAGACCUCAA | 5085 | 2309-2331 |
| | UU | | | AGA | | |
| AD-1732335 | UAGGCUUUACCUGUUCCCU | 4951 | 2338-2358 | AGAGGGAACAGGUAAAGCCU | 5086 | 2336-2358 |
| | CU | | | AGG | | |
| AD-1732353 | UCUGUAAUCCCUCCAAAAG | 4952 | 2356-2376 | AUCUUUUGGAGGGAUUACAG | 5087 | 2354-2376 |
| | AU | | | AGG | | |
| AD-1732372 | AUGAGACAGAUCUAUGCUU | 4953 | 2375-2395 | ACAAGCAUAGAUCUGUCUCA | 5088 | 2373-2395 |
| | GU | | | UCU | | |
| AD-1732389 | UUGGUCAUCCAGUAAACUG | 4954 | 2392-2412 | AUCAGUUUACUGGAUGACCA | 5089 | 2390-2412 |
| | AU | | | AGC | | |
| AD-1732415 | GUGGGCACGCAAGUGUGGG | 4955 | 2418-2438 | AUCCCACACUUGCGUGCCCA | 5090 | 2416-2438 |
| | AU | | | CAG | | |
| AD-1732440 | AGGCAUGCUCAGAGCUGGC | 4956 | 2443-2463 | AAGCCAGCUCUGAGCAUGCC | 5091 | 2441-2463 |
| | UU | | | UCU | | |
| AD-1732469 | UCUGACUUGCCUUCCUUUC | 4957 | 2472-2492 | AUGAAAGGAAGGCAAGUCAG | 5092 | 2470-2492 |
| | AU | | | AGG | | |
| AD-1732479 | UGCUCCACCCAGGAGUCCU | 4958 | 2503-2523 | ACAGGACUCCUGGGUGGAGC | 5093 | 2501-2523 |
| | GU | | | ACU | | |
| AD-1732500 | CUGGAAGCUGGAAUGGGCA | 4959 | 2524-2544 | AUUGCCCAUUCCAGCUUCCA | 5094 | 2522-2544 |
| | AU | | | GGC | | |
| AD-1732520 | GGGCUGCUGGAGUGGGACA | 4960 | 2544-2564 | ACUGUCCCACUCCAGCAGCC | 5095 | 2542-2564 |
| | GU | | | CUU | | |
| AD-1732533 | CAGGGAGAAGAGGAAGGCC | 4961 | 2561-2581 | AAGGCCUUCCUCUUCUCCCU | 5096 | 2559-2581 |
| | UU | | | GUC | | |
| AD-1732549 | GCCUGGAUGAGGAGAGGGU | 4962 | 2577-2597 | ACACCCUCUCCUCAUCCAGG | 5097 | 2575-2597 |
| | GU | | | CCU | | |
| AD-1732565 | GUGGCAUUUGCUCUGAGAC | 4963 | 2594-2614 | AAGUCUCAGAGCAAAUGCCA | 5098 | 2592-2614 |
| | UU | | | CCC | | |
| AD-1732584 | UGGGUCCUUUUUAGACCUU | 4964 | 2613-2633 | AAAAGGUCUAAAAAGGACCC | 5099 | 2611-2633 |
| | UU | | | AGU | | |
| AD-1732604 | CUCCCUUUGGCUGGACAGU | 4965 | 2654-2674 | AGACUGUCCAGCCAAAGGGA | 5100 | 2652-2674 |
| | CU | | | GGA | | |
| AD-1732625 | UGAACCAUGAGGUCGAUAA | 4966 | 2675-2695 | AAUUAUCGACCUCAUGGUUC | 5101 | 2673-2695 |
| | UU | | | AGG | | |
| AD-1732644 | UGUCUGCAGCCCAAGGCCG | 4967 | 2694-2714 | AUCGGCCUUGGGCUGCAGAC | 5102 | 2692-2714 |
| | AU | | | AUU | | |
| AD-1732661 | CGAGUUUGCGCAAAACCCA | 4968 | 2711-2731 | AAUGGGUUUUGCGCAAACUC | 5103 | 2709-2731 |
| | UU | | | GGC | | |
| AD-1732677 | CCAUGUGUUCUUUGGUAAA | 4969 | 2727-2747 | AGUUUACCAAAGAACACAUG | 5104 | 2725-2747 |
| | CU | | | GGU | | |
| AD-1732696 | CGUGAUGUCUGUGUUUGCU | 4970 | 2746-2766 | AGAGCAAACACAGACAUCAC | 5105 | 2744-2766 |
| | CU | | | GUU | | |
| AD-1732708 | CUCCUAUGAGGGUAAGAGG | 4971 | 2779-2799 | AACCUCUUACCCUCAUAGGA | 5106 | 2777-2799 |
| | UU | | | GGG | | |
| AD-1732728 | CCCUGAAAUAGGAACCCUA | 4972 | 2799-2819 | ACUAGGGUUCCUAUUUCAGG | 5107 | 2797-2819 |
| | GU | | | GAC | | |
| AD-1732744 | CUAGAGGAGAAAGUCUGAA | 4973 | 2815-2835 | AUUUCAGACUUUCUCCUCUA | 5108 | 2813-2835 |
| | AU | | | GGG | | |
| AD-1732757 | GACUGUAAAUCUGAGCUUG | 4974 | 2849-2869 | AUCAAGCUCAGAUUUACAGU | 5109 | 2847-2869 |
| | AU | | | CCC | | |
| AD-1732786 | GAGCAACCCAUGGAAGUUA | 4975 | 2878-2898 | AAUAACUUCCAUGGGUUGCU | 5110 | 2876-2898 |
| | UU | | | CAG | | |
| AD-1732807 | CCACCUUUGACUUGAGGAG | 4976 | 2899-2919 | AUCUCCUCAAGUCAAAGGUG | 5111 | 2897-2919 |
| | AU | | | GGA | | |
| AD-1732827 | CCUUCAUCUAAGGAGAAUC | 4977 | 2919-2939 | AAGAUUCUCCUUAGAUGAAG | 5112 | 2917-2939 |
| | UU | | | GUC | | |
| AD-1732849 | GGAGGCCUUCUGGUGUCUC | 4978 | 2941-2961 | AGGAGACACCAGAAGGCCUC | 5113 | 2939-2961 |
| | CU | | | CUU | | |
| AD-1732865 | UCCCAAUUACAGCUUAGUC | 4979 | 2995-3015 | AAGACUAAGCUGUAAUUGGG | 5114 | 2993-3015 |
| | UU | | | AAG | | |
| AD-1732881 | GUCUCCAGGGCUAGGACUG | 4980 | 3011-3031 | ACCAGUCCUAGCCCUGGAGA | 5115 | 3009-3031 |
| | GU | | | CUA | | |
| AD-1732885 | AAGCAAAGUGAGUCAUUCA | 4981 | 3035-3055 | AGUGAAUGACUCACUUUGCU | 5116 | 3033-3055 |
| | CU | | | UUA | | |
| AD-1732890 | GUGGUGAACAAUUUAUUAA | 4982 | 3079-3099 | AAUUAAUAAAUUGUUCACCA | 5117 | 3077-3099 |
| | UU | | | CCC | | |
| AD-1732908 | UAGGACUUUAAUGCAAUAU | 4983 | 3104-3124 | AAAUAUUGCAUUAAAGUCCU | 5118 | 3102-3124 |
| | UU | | | AUC | | |
| AD-1732940 | GAACAAAAUAGCCUACUUU | 4984 | 3160-3180 | AAAAAGUAGGCUAUUUUGU | 5119 | 3158-3180 |
| | UU | | | UCAU | | |

TABLE 17
Modified Sense and Antisense Strand Sequences of INHBC dsRNA Agents Comprising a GalNAc Derivative Targeting Ligand

| | | SEQ | | SEQ | | SEQ |
|---|---|---|---|---|---|---|
| Duplex | | ID | | ID | | ID |
| Name | Sense Sequence 5′ to 3′ | NO: | Sense Sequence 5′ to 3′ | NO: | mRNA Target Sequence 5′ to 3′ | NO: |
| AD- | uscsuuccAfgGfGfCfcucuggca | 5120 | asCfsugcCfaGfAfggccCfuGfgaaga | 5255 | CUUCUUCCAGGGCCUCUGG | 5390 |
| 1730493 | guL96 | | sasg | | CAGC | |
| AD- | csasggacAfgAfGfUfugagaccac | 5121 | asGfsuggUfcUfCfaacuCfuGfuccug | 5256 | GCCAGGACAGAGUUGAGAC | 5391 |
| 1730512 | uL96 | | sgsc | | CACA | |
| AD- | csascagcUfgUfUfGfagacccuga | 5122 | asUfscagGfgUfCfucaaCfaGfcugug | 5257 | ACCACAGCUGUUGAGACCC | 5392 |
| 1730529 | uL96 | | sgsu | | UGAG | |
| AD- | gsasgucuGfuAfUfUfgcucaaga | 5123 | asUfsucuUfgAfGfcaauAfcAfgacuc | 5258 | CUGAGUCUGUAUUGCUCAA | 5393 |
| 1730554 | auL96 | | sasg | | GAAG | |
| AD- | usgsaccuCfcUfCfAfuugcuucu | 5124 | asCfsagaAfgCfAfaugaGfgAfgguca | 5259 | AAUGACCUCCUCAUUGCUU | 5394 |
| 1730570 | guL96 | | susu | | CUGG | |
| AD- | usgsgccuUfuCfUfCfcuccuggc | 5125 | asAfsgccAfgGfAfggagAfaAfggcc | 5260 | UCUGGCCUUUCUCCUCCUG | 5395 |
| 1730588 | uuL96 | | asgsa | | GCUC | |
| AD- | csusccaaCfcAfCfAfguggccacu | 5126 | asAfsgugGfcCfAfcuguGfgUfugga | 5261 | GGCUCCAACCACAGUGGCC | 5396 |
| 1730606 | uL96 | | gscsc | | ACUC | |
| AD- | gscsggucAfgUfGfUfccagcaug | 5127 | asAfscauGfcUfGfgacaCfuGfaccgc | 5262 | UGGCGGUCAGUGUCCAGCA | 5397 |
| 1730636 | uuL96 | | scsa | | UGUG | |
| AD- | ascscuugGfaAfCfUfggagagcca | 5128 | asUfsggcUfcUfCfcaguUfcCfaaggu | 5263 | CCACCUUGGAACUGGAGAG | 5398 |
| 1730643 | uL96 | | sgsg | | CCAG | |
| AD- | gsasgcugCfuUfCfUfugaucugg | 5129 | asGfsccaGfaUfCfaagaAfgCfagcuc | 5264 | GGGAGCUGCUUCUUGAUCU | 5399 |
| 1730667 | cuL96 | | scsc | | GGCC | |
| AD- | asgsaagcAfuCfUfUfggacaagcu | 5130 | asAfsgcuUfgUfCfcaagAfuGfcuuc | 5265 | AGAGAAGCAUCUUGGACAA | 5400 |
| 1730691 | uL96 | | uscsu | | GCUG | |
| AD- | cscsaacaCfuGfAfAfccgcccugu | 5131 | asAfscagGfgCfGfguucAfgUfguug | 5266 | GCCCAACACUGAACCGCCCU | 5401 |
| 1730726 | uL96 | | gsgsc | | GUG | |
| AD- | csasgagcUfgCfUfUfugaggacu | 5132 | asCfsaguCfcUfCfaaagCfaGfcucug | 5267 | UCCAGAGCUGCUUUGAGGA | 5402 |
| 1730749 | gul96 | | sgsa | | CUGC | |
| AD- | ascsugcaCfuGfCfAfgcaccucca | 5133 | asUfsggaGfgUfGfcugcAfgUfgcag | 5268 | GGACUGCACUGCAGCACCU | 5403 |
| 1730765 | uL96 | | uscsc | | CCAC | |
| AD- | gscsacuuCfuAfGfAfggacaacag | 5134 | asCfsuguUfgUfCfcucuAfgAfagug | 5269 | GGGCACUUCUAGAGGACAA | 5404 |
| 1730769 | uL96 | | cscsc | | CAGG | |
| AD- | gsasacagGfaAfUfGfugaaaucau | 5135 | asAfsugaUfuUfCfacauUfcCfuguuc | 5270 | GGGAACAGGAAUGUGAAAU | 5405 |
| 1730790 | uL96 | | scsc | | CAUC | |
| AD- | csasucagCfuUfUfGfcugagacag | 5136 | asCfsuguCfuCfAfgcaaAfgCfugaug | 5271 | AUCAUCAGCUUUGCUGAGA | 5406 |
| 1730807 | uL96 | | sasu | | CAGG | |
| AD- | asgsgccuCfuCfCfAfccaucaacc | 5137 | asGfsguuGfaUfGfguggAfgAfggcc | 5272 | ACAGGCCUCUCCACCAUCAA | 5407 |
| 1730825 | uL96 | | usgsu | | CCA | |
| AD- | asasccagAfcUfCfGfucuugauuu | 5138 | asAfsaauCfaAfGfacgaGfuCfugguu | 5273 | UCAACCAGACUCGUCUUGA | 5408 |
| 1730841 | uL96 | | sgsa | | UUUU | |
| AD- | asusuuucAfcUfUfCfuccucuga | 5139 | asAfsucaGfaGfGfagaaGfuGfaaaau | 5274 | UGAUUUUCACUUCUCCUCU | 5409 |
| 1730857 | uuL96 | | scsa | | GAUA | |
| AD- | usgsauagAfaCfUfGfcuggugac | 5140 | asUfsgucAfcCfAfgcagUfuCfuauca | 5275 | UCUGAUAGAACUGCUGGUG | 5410 |
| 1730873 | auL96 | | sgsa | | ACAG | |
| AD- | asgsucucAfuGfUfUfcuuugugc | 5141 | asUfsgcaCfaAfAfgaacAfuGfagacu | 5276 | CCAGUCUCAUGUUCUUUGU | 5411 |
| 1730910 | auL96 | | sgsg | | GCAG | |
| AD- | usgscagcUfcCfCfUfuccaauacc | 5142 | asGfsguaUfuGfGfaaggGfaGfcugc | 5277 | UGUGCAGCUCCCUUCCAAU | 5412 |
| 1730926 | uL96 | | ascsa | | ACCA | |
| AD- | usasccacUfuGfGfAfccuugaaag | 5143 | asCfsuuuCfaAfGfguccAfaGfuggu | 5278 | AAUACCACUUGGACCUUGA | 5413 |
| 1730942 | uL96 | | asusu | | AAGU | |
| AD- | asasagugAfgAfGfUfccuugugc | 5144 | asAfsgcaCfaAfGfgacuCfuCfacuuu | 5279 | UGAAAGUGAGAGUCCUUGU | 5414 |
| 1730958 | uuL96 | | scsa | | GCUG | |
| AD- | cscsacauAfaUfAfCfcaaccucac | 5145 | asGfsugaGfgUfUfgguaUfuAfugug | 5280 | GUCCACAUAAUACCAACCU | 5415 |
| 1730982 | uL96 | | gsasc | | CACC | |
| AD- | ususggcuAfcUfCfAfguaccugc | 5146 | asAfsgcaGfgUfAfcugaGfuAfgccaa | 5281 | CCUUGGCUACUCAGUACCU | 5416 |
| 1731003 | uuL96 | | sgsg | | GCUG | |
| AD- | usgscuggAfgGfUfGfgaugccag | 5147 | asAfscugGfcAfUfccacCfuCfcagca | 5282 | CCUGCUGGAGGUGGAUGCC | 5417 |
| 1731019 | uuL96 | | sgsg | | AGUG | |
| AD- | csasguggCfuGfGfCfaucaacucc | 5148 | asGfsgagUfuGfAfugccAfgCfcacu | 5283 | GCCAGUGGCUGGCAUCAAC | 5418 |
| 1731035 | uL96 | | gsgsc | | UCCC | |
| AD- | cscsugaaGfcUfCfAfagcugccug | 5149 | asCfsaggCfaGfCfuugaGfcUfucagg | 5284 | GGCCUGAAGCUCAAGCUGC | 5419 |
| 1731042 | uL96 | | scsc | | CUGC | |
| AD- | gsasgcugGfuAfCfUfugaaggcc | 5150 | asUfsggcCfuUfCfaaguAfcCfagcuc | 5285 | UGGAGCUGGUACUUGAAGG | 5420 |
| 1731064 | auL96 | | scsa | | CCAG | |
| AD- | gscsccagAfgCfUfCfagucauccu | 5151 | asAfsggaUfgAfCfugagCfuCfuggg | 5286 | UAGCCCAGAGCUCAGUCAU | 5421 |
| 1731088 | uL96 | | csusa | | CCUG | |
| AD- | usgscccaUfaGfGfCfcuuuugug | 5152 | asCfscacAfaAfAfggccUfaUfgggca | 5287 | GCUGCCCAUAGGCCUUUUG | 5422 |
| 1731117 | gul96 | | sgsc | | UGGC | |
| AD- | usgsgcagCfcCfGfGfgugagagu | 5153 | asAfsacuCfuCfAfcccgGfgCfugcca | 5288 | UGUGGCAGCCCGGGUGAGA | 5423 |
| 1731134 | uuL96 | | scsa | | GUUG | |
| AD- | gscsaaacAfcCfAfGfauucaccga | 5154 | asUfscggUfgAfAfucugGfuGfuuug | 5289 | GGGCAAACACCAGAUUCAC | 5424 |
| 1731137 | uL96 | | cscsc | | CGAC | |
| AD- | ascsgaggCfaUfCfGfacugccaag | 5155 | asCfsuugGfcAfGfucgaUfgCfcucg | 5290 | CGACGAGGCAUCGACUGCC | 5425 |
| 1731156 | uL96 | | uscsg | | AAGG | |
| AD- | asasggagGfgUfCfCfaggaugug | 5156 | asGfscacAfuCfCfuggaCfcCfuccuu | 5291 | CCAAGGAGGGUCCAGGAUG | 5426 |
| 1731173 | cuL96 | | sgsg | | UGCU | |
| AD- | gsusgcugUfcGfAfCfaagaguuu | 5157 | asAfsaaaCfuCfUfugucGfaCfagcac | 5292 | AUGUGCUGUCGACAAGAGU | 5427 |
| 1731189 | uuL96 | | sasu | | UUUU | |
| AD- | ususuuuuGfuGfGfAfcuuccgug | 5158 | asUfscacGfgAfAfguccAfcAfaaaaa | 5293 | AGUUUUUUGUGGACUUCCG | 5428 |
| 1731205 | auL96 | | scsu | | UGAG | |
| AD- | gscsacgaCfuGfGfAfucauccagc | 5159 | asGfscugGfaUfGfauccAfgUfcgug | 5294 | UGGCACGACUGGAUCAUCC | 5429 |
| 1731234 | uL96 | | cscsa | | AGCC | |
| AD- | csgsccauGfaAfCfUfucugcauag | 5160 | asCfsuauGfcAfGfaaguUfcAfuggc | 5295 | UACGCCAUGAACUUCUGCA | 5430 |
| 1731264 | uL96 | | gsusa | | UAGG | |
| AD- | usgscccaCfuAfCfAfcauagcagg | 5161 | asCfscugCfuAfUfguguAfgUfgggc | 5296 | AGUGCCCACUACACAUAGC | 5431 |
| 1731289 | uL96 | | ascsu | | AGGC | |
| AD- | gsgscaugCfcUfGfGfuauugcug | 5162 | asGfscagCfaAfUfaccaGfgCfaugcc | 5297 | CAGGCAUGCCUGGUAUUGC | 5432 |
| 1731307 | cuL96 | | susg | | UGCC | |
| AD- | csusgccuCfcUfUfUfcacacugca | 5163 | asUfsgcaGfuGfUfgaaaGfgAfggca | 5298 | UGCUGCCUCCUUUCACACU | 5433 |
| 1731323 | uL96 | | gscsa | | GCAG | |
| AD- | gscsagugCfuCfAfAfucuucucaa | 5164 | asUfsugaGfaAfGfauugAfgCfacug | 5299 | CUGCAGUGCUCAAUCUUCU | 5434 |
| 1731340 | uL96 | | csasg | | CAAG | |
| AD- | asasggccAfaCfAfCfagcugcagg | 5165 | asCfscugCfaGfCfugugUfuGfgccu | 5300 | UCAAGGCCAACACAGCUGC | 5435 |
| 1731358 | uL96 | | usgsa | | AGGC | |
| AD- | gscsucauGfcUfGfUfguacccacg | 5166 | asCfsgugGfgUfAfcacaGfcAfugagc | 5301 | GGGCUCAUGCUGUGUACCC | 5436 |
| 1731371 | uL96 | | scsc | | ACGG | |
| AD- | gsuscucuGfcUfCfUfauuaugaca | 5167 | asUfsgucAfuAfAfuagaGfcAfgaga | 5302 | CUGUCUCUGCUCUAUUAUG | 5437 |
| 1731380 | uL96 | | csasg | | ACAG | |
| AD- | gsascaggGfaCfAfGfcaacauugu | 5168 | asAfscaaUfgUfUfgcugUfcCfcuguc | 5303 | AUGACAGGGACAGCAACAU | 5438 |
| 1731398 | uL96 | | sasu | | UGUC | |
| AD- | usgsucaaGfaCfUfGfacauaccug | 5169 | asCfsaggUfaUfGfucagUfcUfugaca | 5304 | AUUGUCAAGACUGACAUAC | 5439 |
| 1731415 | uL96 | | sasu | | CUGA | |
| AD- | cscsugacAfuGfGfUfaguagagg | 5170 | asGfsccuCfuAfCfuaccAfuGfucagg | 5305 | UACCUGACAUGGUAGUAGA | 5440 |
| 1731431 | cuL96 | | susa | | GGCC | |
| AD- | asgsuuagUfcUfAfUfguguggua | 5171 | asAfsuacCfaCfAfcauaGfaCfuaacu | 5306 | GCAGUUAGUCUAUGUGUGG | 5441 |
| 1731457 | uuL96 | | sgsc | | UAUG | |
| AD- | cscsaaggUfuGfCfAfugggaaaac | 5172 | asGfsuuuUfcCfCfaugcAfaCfcuugg | 5307 | GCCCAAGGUUGCAUGGGAA | 5442 |
| 1731484 | uL96 | | sgsc | | AACA | |
| AD- | ascsagaaGfuGfCfAfcuuccuuga | 5173 | asUfscaaGfgAfAfgugcAfcUfucug | 5308 | CUACAGAAGUGCACUUCCU | 5443 |
| 1731492 | uL96 | | usasg | | UGAG | |
| AD- | asgsaggaGfgGfAfAfugaccuca | 5174 | asAfsugaGfgUfCfauucCfcUfccucu | 5309 | UGAGAGGAGGGAAUGACCU | 5444 |
| 1731511 | uuL96 | | scsa | | CAUU | |
| AD- | uscsauucUfcUfGfUfccagaaugu | 5175 | asAfscauUfcUfGfgacaGfaGfaauga | 5310 | CCUCAUUCUCUGUCCAGAA | 5445 |
| 1731527 | uL96 | | sgsg | | UGUG | |
| AD- | gsusggacUfcCfCfUfcuuccugag | 5176 | asCfsucaGfgAfAfgaggGfaGfuccac | 5311 | AUGUGGACUCCCUCUUCCU | 5446 |
| 1731545 | uL96 | | sasu | | GAGC | |
| AD- | gsasgcauCfuUfAfUfggaaauuac | 5177 | asGfsuaaUfuUfCfcauaAfgAfugcuc | 5312 | CUGAGCAUCUUAUGGAAAU | 5447 |
| 1731562 | uL96 | | sasg | | UACC | |
| AD- | csasccuuUfgAfCfUfugaagaaac | 5178 | asGfsuuuCfuUfCfaaguCfaAfaggu | 5313 | CCCACCUUUGACUUGAAGA | 5448 |
| 1731564 | uL96 | | gsgsg | | AACC | |
| AD- | csusucauCfuAfAfAfgcaagucac | 5179 | asGfsugaCfuUfGfcuuuAfgAfugaa | 5314 | ACCUUCAUCUAAAGCAAGU | 5449 |
| 1731584 | uL96 | | gsgsu | | CACU | |
| AD- | uscsacugUfgCfCfAfucuuccuga | 5180 | asUfscagGfaAfGfauggCfaCfaguga | 5315 | AGUCACUGUGCCAUCUUCC | 5450 |
| 1731600 | uL96 | | scsu | | UGAC | |
| AD- | gsasccacUfaCfCfCfucuuuccua | 5181 | asUfsaggAfaAfGfagggUfaGfuggu | 5316 | CUGACCACUACCCUCUUUCC | 5451 |
| 1731618 | uL96 | | csasg | | UAG | |
| AD- | uscscaucCfcGfCfUfaguccaucc | 5182 | asGfsgauGfgAfCfuagcGfgGfaugg | 5317 | AGUCCAUCCCGCUAGUCCA | 5452 |
| 1731646 | uL96 | | ascsu | | UCCC | |
| AD- | ascsccauCfuCfCfAfaccaugagc | 5183 | asGfscucAfuGfGfuuggAfgAfuggg | 5318 | AGACCCAUCUCCAACCAUG | 5453 |
| 1731671 | uL96 | | uscsu | | AGCA | |
| AD- | gsasgcaaUfgCfCfAfucugguucc | 5184 | asGfsgaaCfcAfGfauggCfaUfugcuc | 5319 | AUGAGCAAUGCCAUCUGGU | 5454 |
| 1731687 | uL96 | | sasu | | UCCC | |
| AD- | uscsccagGfcAfAfAfgacacccuu | 5185 | asAfsaggGfuGfUfcuuuGfcCfuggg | 5320 | GUUCCCAGGCAAAGACACC | 5455 |
| 1731704 | uL96 | | asasc | | CUUA | |
| AD- | cscsuuagCfuCfAfCfcuuuaauag | 5186 | asCfsuauUfaAfAfggugAfgCfuaag | 5321 | ACCCUUAGCUCACCUUUAA | 5456 |
| 1731720 | uL96 | | gsgsu | | UAGA | |
| AD- | ascsccacUfaUfGfCfcuuccuguc | 5187 | asGfsacaGfgAfAfggcaUfaGfuggg | 5322 | UAACCCACUAUGCCUUCCU | 5457 |
| 1731728 | uL96 | | ususa | | GUCC | |
| AD- | gsusccuuUfcUfAfCfucaauggu | 5188 | asGfsaccAfuUfGfaguaGfaAfaggac | 5323 | CUGUCCUUUCUACUCAAUG | 5458 |
| 1731745 | cuL96 | | sasg | | GUCC | |
| AD- | uscscaagAfuGfAfGfuugacacaa | 5189 | asUfsuguGfuCfAfacucAfuCfuugg | 5324 | ACUCCAAGAUGAGUUGACA | 5459 |
| 1731750 | uL96 | | asgsu | | CAAC | |
| AD- | csasauuuUfuGfUfGfgaucucca | 5190 | asCfsuggAfgAfUfccacAfaAfaauug | 5325 | CCCAAUUUUUGUGGAUCUC | 5460 |
| 1731753 | guL96 | | sgsg | | CAGA | |
| AD- | uscsuuugGfaUfUfCfaccaaaguu | 5191 | asAfsacuUfuGfGfugaaUfcCfaaaga | 5326 | CUUCUUUGGAUUCACCAAA | 5461 |
| 1731782 | uL96 | | sasg | | GUUU | |
| AD- | usgscccaAfaAfUfAfgaggcuuac | 5192 | asGfsuaaGfcCfUfcuauUfuUfgggca | 5327 | GCUGCCCAAAAUAGAGGCU | 5462 |
| 1731813 | uL96 | | sgsc | | UACC | |
| AD- | csusguccUfuCfUfUfaguugucc | 5193 | asUfsggaCfaAfCfuaagAfaGfgacag | 5328 | CCCUGUCCUUCUUAGUUGU | 5463 |
| 1731819 | auL96 | | sgsg | | CCAG | |
| AD- | gsasacuaCfuAfAfAfgcucucuu | 5194 | asAfsaagAfgAfGfcuuuAfgUfaguu | 5329 | GUGAACUACUAAAGCUCUC | 5464 |
| 1731842 | uuL96 | | csasc | | UUUG | |
| AD- | csusuugcAfuAfCfCfuucauccau | 5195 | asAfsuggAfuGfAfagguAfuGfcaaa | 5330 | CUCUUUGCAUACCUUCAUC | 5465 |
| 1731858 | uL96 | | gsasg | | CAUU | |
| AD- | cscsauuuUfuUfGfUfccuucucu | 5196 | asCfsagaGfaAfGfgacaAfaAfaaugg | 5331 | AUCCAUUUUUUGUCCUUCU | 5466 |
| 1731874 | guL96 | | sasu | | CUGC | |
| AD- | gscscuuuCfuCfUfAfugcccuuaa | 5197 | asUfsuaaGfgGfCfauagAfgAfaaggc | 5332 | CUGCCUUUCUCUAUGCCCU | 5467 |
| 1731890 | uL96 | | sasg | | UAAG | |
| AD- | ususaaggGfcUfGfAfcuugccug | 5198 | asUfscagGfcAfAfgucaGfcCfcuuaa | 5333 | CCUUAAGGGCUGACUUGCC | 5468 |
| 1731906 | auL96 | | sgsg | | UGAG | |
| AD- | usgsagcuCfuAfUfCfaccugagcu | 5199 | asAfsgcuCfaGfGfugauAfgAfgcuc | 5334 | CCUGAGCUCUAUCACCUGA | 5469 |
| 1731923 | uL96 | | asgsg | | GCUC | |
| AD- | uscsuggcUfuCfCfUfgcugaggu | 5200 | asGfsaccUfcAfGfcaggAfaGfccaga | 5335 | CCUCUGGCUUCCUGCUGAG | 5470 |
| 1731932 | cuL96 | | sgsg | | GUCA | |
| AD- | ususucuuAfuCfCfCfuguucccu | 5201 | asGfsaggGfaAfCfagggAfuAfagaaa | 5336 | CAUUUCUUAUCCCUGUUCC | 5471 |
| 1731958 | cuL96 | | susg | | CUCU | |
| AD- | uscsugucUfaGfGfUfgucauggu | 5202 | asAfsaccAfuGfAfcaccUfaGfacaga | 5337 | UCUCUGUCUAGGUGUCAUG | 5472 |
| 1731978 | uuL96 | | sgsa | | GUUC | |
| AD- | uscsugugUfaAfCfUfguggcuau | 5203 | asAfsauaGfcCfAfcaguUfaCfacaga | 5338 | GUUCUGUGUAACUGUGGCU | 5473 |
| 1731997 | uuL96 | | sasc | | AUUC | |
| AD- | usasuucuGfuGfUfCfccuacacua | 5204 | asUfsaguGfuAfGfggacAfcAfgaau | 5339 | GCUAUUCUGUGUCCCUACA | 5474 |
| 1732013 | uL96 | | asgsc | | CUAC | |
| AD- | gscscuacAfuUfCfUfgauauaacu | 5205 | asAfsguuAfuAfUfcagaAfuGfuagg | 5340 | CUGCCUACAUUCUGAUAUA | 5475 |
| 1732031 | uL96 | | csasg | | ACUG | |
| AD- | cscsuaaaGfgCfUfUfucuaucuug | 5206 | asCfsaagAfuAfGfaaagCfcUfuuagg | 5341 | GUCCUAAAGGCUUUCUAUC | 5476 |
| 1732048 | uL96 | | sasc | | UUGC | |
| AD- | csuscaacAfuCfUfCfauacugguu | 5207 | asAfsaccAfgUfAfugagAfuGfuuga | 5342 | GCCUCAACAUCUCAUACUG | 5477 |
| 1732062 | uL96 | | gsgsc | | GUUC | |
| AD- | gsgsuuccCfuUfAfAfcucugccu | 5208 | asUfsaggCfaGfAfguuaAfgGfgaacc | 5343 | CUGGUUCCCUUAACUCUGC | 5478 |
| 1732078 | auL96 | | sasg | | CUAU | |
| AD- | cscsuauaCfcUfCfUfguaaauaau | 5209 | asAfsuuaUfuUfAfcagaGfgUfauag | 5344 | UGCCUAUACCUCUGUAAAU | 5479 |
| 1732094 | uL96 | | gscsa | | AAUU | |
| AD- | usasauucCfuUfCfAfcuaaguucu | 5210 | asAfsgaaCfuUfAfgugaAfgGfaauu | 5345 | AAUAAUUCCUUCACUAAGU | 5480 |
| 1732110 | uL96 | | asusu | | UCUC | |
| AD- | asasagucCfuCfUfAfucuccuaca | 5211 | asUfsguaGfgAfGfauagAfgGfacuu | 5346 | GAAAAGUCCUCUAUCUCCU | 5481 |
| 1732157 | uL96 | | ususc | | ACAA | |
| AD- | usascaagGfgCfCfCfuaacuggca | 5212 | asUfsgccAfgUfUfagggCfcCfuugu | 5347 | CCUACAAGGGCCCUAACUG | 5482 |
| 1732173 | uL96 | | asgsg | | GCAC | |
| AD- | csasgaugAfcAfCfAfgagccugcc | 5213 | asGfsgcaGfgCfUfcuguGfuCfaucu | 5348 | CCCAGAUGACACAGAGCCU | 5483 |
| 1732176 | uL96 | | gsgsg | | GCCU | |
| AD- | csusgcuuAfuGfCfUfguagucug | 5214 | asGfscagAfcUfAfcagcAfuAfagcag | 5349 | GCCUGCUUAUGCUGUAGUC | 5484 |
| 1732195 | cuL96 | | sgsc | | UGCC | |
| AD- | ascsucugCfuGfUfCfucuucacau | 5215 | asAfsuguGfaAfGfagacAfgCfagag | 5350 | CUACUCUGCUGUCUCUUCA | 5485 |
| 1732217 | uL96 | | usasg | | CAUG | |
| AD- | asusggucUfcCfUfCfagaacugaa | 5216 | asUfsucaGfuUfCfugagGfaGfaccau | 5351 | ACAUGGUCUCCUCAGAACU | 5486 |
| 1732235 | uL96 | | sgsu | | GAAC | |
| AD- | asascuauUfgUfAfUfccaucucac | 5217 | asGfsugaGfaUfGfgauaCfaAfuagu | 5352 | UGAACUAUUGUAUCCAUCU | 5487 |
| 1732253 | uL96 | | uscsa | | CACA | |
| AD- | uscsacacUfuUfAfUfgccucuucu | 5218 | asAfsgaaGfaGfGfcauaAfaGfuguga | 5353 | UCUCACACUUUAUGCCUCU | 5488 |
| 1732269 | uL96 | | sgsa | | UCUU | |
| AD- | asusccuuCfcAfGfAfaccaucuuu | 5219 | asAfsaagAfuGfGfuucuGfgAfagga | 5354 | CCAUCCUUCCAGAACCAUCU | 5489 |
| 1732291 | uL96 | | usgsg | | UUG | |
| AD- | ususugagGfuCfUfCfauggcuaa | 5220 | asAfsuuaGfcCfAfugagAfcCfucaaa | 5355 | UCUUUGAGGUCUCAUGGCU | 5490 |
| 1732308 | uuL96 | | sgsa | | AAUA | |
| AD- | usasggcuUfuAfCfCfuguucccu | 5221 | asGfsaggGfaAfCfagguAfaAfgccua | 5356 | CCUAGGCUUUACCUGUUCC | 5491 |
| 1732335 | cuL96 | | sgsg | | CUCU | |
| AD- | uscsuguaAfuCfCfCfuccaaaaga | 5222 | asUfscuuUfuGfGfagggAfuUfacag | 5357 | CCUCUGUAAUCCCUCCAAA | 5492 |
| 1732353 | uL96 | | asgsg | | AGAU | |
| AD- | asusgagaCfaGfAfUfcuaugcuu | 5223 | asCfsaagCfaUfAfgaucUfgUfcucau | 5358 | AGAUGAGACAGAUCUAUGC | 5493 |
| 1732372 | gul96 | | scsu | | UUGG | |
| AD- | ususggucAfuCfCfAfguaaacug | 5224 | asUfscagUfuUfAfcuggAfuGfaccaa | 5359 | GCUUGGUCAUCCAGUAAAC | 5494 |
| 1732389 | auL96 | | sgsc | | UGAC | |
| AD- | gsusgggcAfcGfCfAfaguguggg | 5225 | asUfscccAfcAfCfuugcGfuGfcccac | 5360 | CUGUGGGCACGCAAGUGUG | 5495 |
| 1732415 | auL96 | | sasg | | GGAG | |
| AD- | asgsgcauGfcUfCfAfgagcuggc | 5226 | asAfsgccAfgCfUfcugaGfcAfugccu | 5361 | AGAGGCAUGCUCAGAGCUG | 5496 |
| 1732440 | uuL96 | | scsu | | GCUG | |
| AD- | uscsugacUfuGfCfCfuuccuuuca | 5227 | asUfsgaaAfgGfAfaggcAfaGfucaga | 5362 | CCUCUGACUUGCCUUCCUU | 5497 |
| 1732469 | uL96 | | sgsg | | UCAC | |
| AD- | usgscuccAfcCfCfAfggaguccug | 5228 | asCfsaggAfcUfCfcuggGfuGfgagca | 5363 | AGUGCUCCACCCAGGAGUC | 5498 |
| 1732479 | uL96 | | scsu | | CUGC | |
| AD- | csusggaaGfcUfGfGfaaugggcaa | 5229 | asUfsugcCfcAfUfuccaGfcUfuccag | 5364 | GCCUGGAAGCUGGAAUGGG | 5499 |
| 1732500 | uL96 | | sgsc | | CAAG | |
| AD- | gsgsgcugCfuGfGfAfgugggaca | 5230 | asCfsuguCfcCfAfcuccAfgCfagccc | 5365 | AAGGGCUGCUGGAGUGGGA | 5500 |
| 1732520 | guL96 | | susu | | CAGG | |
| AD- | csasgggaGfaAfGfAfggaaggcc | 5231 | asAfsggcCfuUfCfcucuUfcUfcccug | 5366 | GACAGGGAGAAGAGGAAGG | 5501 |
| 1732533 | uuL96 | | susc | | CCUG | |
| AD- | gscscuggAfuGfAfGfgagagggu | 5232 | asCfsaccCfuCfUfccucAfuCfcaggc | 5367 | AGGCCUGGAUGAGGAGAGG | 5502 |
| 1732549 | gul96 | | scsu | | GUGG | |
| AD- | gsusggcaUfuUfGfCfucugagac | 5233 | asAfsgucUfcAfGfagcaAfaUfgccac | 5368 | GGGUGGCAUUUGCUCUGAG | 5503 |
| 1732565 | uuL96 | | scsc | | ACUG | |
| AD- | usgsggucCfuUfUfUfuagaccuu | 5234 | asAfsaagGfuCfUfaaaaAfgGfaccca | 5369 | ACUGGGUCCUUUUUAGACC | 5504 |
| 1732584 | uuL96 | | sgsu | | UUUG | |
| AD- | csuscccuUfuGfGfCfuggacaguc | 5235 | asGfsacuGfuCfCfagccAfaAfgggag | 5370 | UCCUCCCUUUGGCUGGACA | 5505 |
| 1732604 | uL96 | | sgsa | | GUCC | |
| AD- | usgsaaccAfuGfAfGfgucgauaa | 5236 | asAfsuuaUfcGfAfccucAfuGfguuc | 5371 | CCUGAACCAUGAGGUCGAU | 5506 |
| 1732625 | uuL96 | | asgsg | | AAUG | |
| AD- | usgsucugCfaGfCfCfcaaggccga | 5237 | asUfscggCfcUfUfgggcUfgCfagaca | 5372 | AAUGUCUGCAGCCCAAGGC | 5507 |
| 1732644 | uL96 | | susu | | CGAG | |
| AD- | csgsaguuUfgCfGfCfaaaacccau | 5238 | asAfsuggGfuUfUfugcgCfaAfacuc | 5373 | GCCGAGUUUGCGCAAAACC | 5508 |
| 1732661 | uL96 | | gsgsc | | CAUG | |
| AD- | cscsauguGfuUfCfUfuugguaaa | 5239 | asGfsuuuAfcCfAfaagaAfcAfcaugg | 5374 | ACCCAUGUGUUCUUUGGUA | 5509 |
| 1732677 | cuL96 | | sgsu | | AACG | |
| AD- | csgsugauGfuCfUfGfuguuugcu | 5240 | asGfsagcAfaAfCfacagAfcAfucacg | 5375 | AACGUGAUGUCUGUGUUUG | 5510 |
| 1732696 | cuL96 | | susu | | CUCA | |
| AD- | csusccuaUfgAfGfGfguaagagg | 5241 | asAfsccuCfuUfAfcccuCfaUfaggag | 5376 | CCCUCCUAUGAGGGUAAGA | 5511 |
| 1732708 | uuL96 | | sgsg | | GGUC | |
| AD- | cscscugaAfaUfAfGfgaacccuag | 5242 | asCfsuagGfgUfUfccuaUfuUfcagg | 5377 | GUCCCUGAAAUAGGAACCC | 5512 |
| 1732728 | uL96 | | gsasc | | UAGA | |
| AD- | csusagagGfaGfAfAfagucugaaa | 5243 | asUfsuucAfgAfCfuuucUfcCfucua | 5378 | CCCUAGAGGAGAAAGUCUG | 5513 |
| 1732744 | uL96 | | gsgsg | | AAAA | |
| AD- | gsascuguAfaAfUfCfugagcuug | 5244 | asUfscaaGfcUfCfagauUfuAfcaguc | 5379 | GGGACUGUAAAUCUGAGCU | 5514 |
| 1732757 | auL96 | | scsc | | UGAG | |
| AD- | gsasgcaaCfcCfAfUfggaaguuau | 5245 | asAfsuaaCfuUfCfcaugGfgUfugcuc | 5380 | CUGAGCAACCCAUGGAAGU | 5515 |
| 1732786 | uL96 | | sasg | | UAUC | |
| AD- | cscsaccuUfuGfAfCfuugaggaga | 5246 | asUfscucCfuCfAfagucAfaAfggug | 5381 | UCCCACCUUUGACUUGAGG | 5516 |
| 1732807 | uL96 | | gsgsa | | AGAC | |
| AD- | cscsuucaUfcUfAfAfggagaaucu | 5247 | asAfsgauUfcUfCfcuuaGfaUfgaagg | 5382 | GACCUUCAUCUAAGGAGAA | 5517 |
| 1732827 | uL96 | | susc | | UCUA | |
| AD- | gsgsaggcCfuUfCfUfggugucuc | 5248 | asGfsgagAfcAfCfcagaAfgGfccucc | 5383 | AAGGAGGCCUUCUGGUGUC | 5518 |
| 1732849 | cuL96 | | susu | | UCCC | |
| AD- | uscsccaaUfuAfCfAfgcuuagucu | 5249 | asAfsgacUfaAfGfcuguAfaUfuggg | 5384 | CUUCCCAAUUACAGCUUAG | 5519 |
| 1732865 | uL96 | | asasg | | UCUC | |
| AD- | gsuscuccAfgGfGfCfuaggacug | 5250 | asCfscagUfcCfUfagccCfuGfgagac | 5385 | UAGUCUCCAGGGCUAGGAC | 5520 |
| 1732881 | gul96 | | susa | | UGGG | |
| AD- | asasgcaaAfgUfGfAfgucauucac | 5251 | asGfsugaAfuGfAfcucaCfuUfugcu | 5386 | UAAAGCAAAGUGAGUCAUU | 5521 |
| 1732885 | uL96 | | ususa | | CACC | |
| AD- | gsusggugAfaCfAfAfuuuauuaa | 5252 | asAfsuuaAfuAfAfauugUfuCfaccac | 5387 | GGGUGGUGAACAAUUUAUU | 5522 |
| 1732890 | uuL96 | | scsc | | AAUC | |
| AD- | usasggacUfuUfAfAfugcaauau | 5253 | asAfsauaUfuGfCfauuaAfaGfuccua | 5388 | GAUAGGACUUUAAUGCAAU | 5523 |
| 1732908 | uuL96 | | susc | | AUUA | |
| AD- | gsasacaaAfaUfAfGfccuacuuuu | 5254 | asAfsaaaGfuAfGfgcuaUfuUfuguu | 5389 | AUGAACAAAAUAGCCUACU | 5524 |
| 1732940 | uL96 | | csasu | | UUUA | |

TABLE 18
Single dose screen for dsRNA agents
targeting INHBE in Hep3b cells

| | INHBE/gapdh | | INHBE/gapdh | |
|---|---|---|---|---|
| | 10 nM | | 1 nM | |
| | Average % | | Average % | |
| | message | | message | |
| Duplex Name | remaining | SD | remaining | SD |
| AD-1657845.1 | 111.502 | 7.324 | 91.264 | 6.933 |
| AD-1657834.1 | 73.740 | 4.726 | 60.700 | 3.044 |
| AD-1657822.1 | 44.254 | 6.361 | 31.710 | 2.225 |
| AD-1657811.1 | 57.311 | 6.952 | 42.989 | 3.234 |
| AD-1657793.1 | 57.109 | 9.054 | 57.657 | 1.772 |
| AD-1657776.1 | 31.680 | 4.646 | 29.169 | 6.609 |
| AD-1657760.1 | 20.839 | 4.152 | 19.336 | 1.898 |
| AD-1657744.1 | 34.473 | 7.839 | 30.596 | 0.788 |
| AD-1657741.1 | 74.347 | 14.701 | 74.106 | 5.550 |
| AD-1657727.1 | 72.423 | 11.797 | 80.936 | 2.697 |
| AD-1657716.1 | 22.638 | 3.700 | 23.912 | 6.962 |
| AD-1657704.1 | 28.552 | 6.159 | 28.798 | 6.146 |
| AD-1657687.1 | 54.039 | 9.112 | 54.137 | 1.783 |
| AD-1657674.1 | 47.061 | 4.830 | 43.380 | 3.109 |
| AD-1657653.1 | 31.466 | 2.523 | 32.508 | 1.736 |
| AD-1657641.1 | 24.346 | 3.640 | 25.044 | 1.349 |
| AD-1657615.1 | 48.959 | 8.120 | 56.602 | 4.347 |
| AD-1657600.1 | 68.423 | 6.167 | 71.585 | 7.687 |
| AD-1657586.1 | 54.978 | 13.599 | 52.685 | 2.375 |
| AD-1657575.1 | 55.241 | 7.227 | 57.979 | 7.288 |
| AD-1657564.1 | 45.901 | 2.503 | 54.016 | 1.990 |
| AD-1657552.1 | 82.298 | 13.007 | 76.189 | 6.130 |
| AD-1657540.1 | 22.575 | 2.322 | 21.798 | 2.756 |
| AD-1657529.1 | 33.762 | 2.785 | 32.357 | 0.747 |
| AD-1657520.1 | 40.770 | 2.904 | 40.577 | 2.140 |
| AD-1657503.1 | 81.097 | 10.061 | 95.267 | 5.483 |
| AD-1657463.1 | 104.817 | 3.003 | 102.395 | 7.680 |
| AD-1657475.1 | 32.866 | 3.805 | 36.728 | 3.060 |
| AD-1657457.1 | 55.309 | 7.525 | 62.481 | 4.757 |
| AD-1657446.1 | 64.718 | 5.426 | 70.633 | 2.386 |
| AD-1657431.1 | 82.429 | 3.640 | 94.677 | 7.863 |
| AD-1657410.1 | 85.127 | 11.511 | 95.982 | 4.312 |
| AD-1657395.1 | 34.606 | 4.035 | 44.299 | 3.633 |
| AD-1657385.1 | 25.376 | 4.571 | 30.272 | 1.859 |
| AD-1657374.1 | 34.005 | 3.832 | 40.414 | 6.995 |
| AD-1657359.1 | 36.416 | 4.539 | 42.496 | 1.445 |
| AD-1657347.1 | 49.056 | 7.109 | 53.442 | 1.120 |
| AD-1657335.1 | 38.944 | 8.573 | 57.606 | 15.402 |
| AD-1657324.1 | 67.982 | 8.933 | 60.199 | 1.498 |
| AD-1657322.1 | 70.552 | 6.423 | 98.389 | 1.565 |
| AD-1657299.1 | 51.716 | 10.063 | 51.866 | 3.171 |
| AD-1657286.1 | 58.362 | 4.990 | 59.650 | 3.084 |
| AD-1657274.1 | 49.867 | 7.374 | 55.066 | 2.475 |
| AD-1657261.1 | 67.171 | 10.247 | 75.283 | 5.022 |
| AD-1657245.1 | 107.148 | 10.325 | 107.609 | 3.856 |
| AD-1657234.1 | 49.466 | 10.471 | 55.763 | 3.564 |
| AD-1657211.1 | 66.217 | 9.659 | 89.842 | 2.170 |
| AD-1657202.1 | 24.284 | 0.920 | 30.464 | 1.354 |
| AD-1657185.1 | 52.769 | 4.360 | 57.078 | 1.715 |
| AD-1657164.1 | 98.337 | 9.509 | 102.454 | 1.987 |
| AD-1657147.1 | 33.650 | 4.858 | 37.393 | 2.032 |
| AD-1657133.1 | 47.322 | 3.533 | 59.465 | 4.976 |
| AD-1657119.1 | 33.540 | 5.318 | 27.596 | 0.347 |
| AD-1657113.1 | 19.963 | 4.057 | 20.771 | 1.598 |
| AD-1657099.1 | 34.125 | 3.942 | 37.359 | 1.925 |
| AD-1657085.1 | 37.089 | 9.439 | 33.790 | 1.346 |
| AD-1657069.1 | 40.171 | 5.158 | 39.186 | 5.693 |
| AD-1657058.1 | 22.085 | 1.950 | 23.312 | 5.043 |
| AD-1657045.1 | 37.085 | 2.715 | 32.953 | 2.922 |
| AD-1657037.1 | 27.931 | 2.008 | 28.287 | 1.070 |
| AD-1657022.1 | 43.503 | 4.641 | 49.124 | 2.709 |
| AD-1657013.1 | 25.911 | 5.728 | 31.816 | 2.016 |
| AD-1656996.1 | 66.202 | 1.771 | 84.949 | 2.887 |
| AD-1656969.1 | 22.138 | 0.831 | 20.535 | 1.620 |
| AD-1656958.1 | 15.865 | 1.070 | 16.371 | 1.040 |
| AD-1656954.1 | 40.878 | 9.408 | 53.844 | 4.173 |
| AD-1656942.1 | 30.879 | 6.184 | 43.036 | 3.002 |
| AD-1656926.1 | 83.175 | 10.550 | 103.354 | 10.184 |
| AD-1656915.1 | 43.426 | 5.790 | 45.498 | 3.633 |
| AD-1656900.1 | 57.647 | 7.574 | 71.612 | 1.628 |
| AD-1656888.1 | 20.414 | 2.786 | 20.645 | 0.527 |
| AD-1656876.1 | 41.507 | 8.799 | 64.531 | 3.928 |
| AD-1656873.1 | 95.455 | 1.755 | 110.427 | 4.031 |
| AD-1656862.1 | 28.261 | 3.728 | 25.877 | 4.127 |
| AD-1656849.1 | 42.736 | 6.226 | 53.757 | 4.118 |
| AD-1656832.1 | 22.284 | 4.193 | 24.704 | 1.507 |
| AD-1656820.1 | 36.282 | 2.484 | 49.723 | 2.973 |
| AD-1656808.1 | 33.056 | 6.984 | 23.344 | 1.171 |
| AD-1656792.1 | 32.327 | 4.602 | 63.303 | 4.848 |
| AD-1656775.1 | 78.089 | 6.399 | 74.958 | 10.131 |
| AD-1656762.1 | 34.695 | 5.445 | 45.070 | 2.460 |
| AD-1656754.1 | 68.025 | 10.751 | 95.733 | 6.112 |
| AD-1656740.1 | 109.099 | 15.708 | 110.169 | 5.851 |
| AD-1656728.1 | 58.629 | 6.016 | 78.387 | 19.524 |
| AD-1656716.1 | 58.703 | 8.377 | 76.566 | 8.896 |
| AD-1656701.1 | 101.804 | 8.079 | 101.812 | 6.138 |
| AD-1656690.1 | 72.753 | 13.083 | 85.011 | 9.161 |
| AD-1656679.1 | 26.622 | 3.632 | 44.404 | 2.181 |
| AD-1656667.1 | 84.688 | 16.798 | 93.321 | 4.608 |
| AD-1656647.1 | 52.261 | 9.437 | 79.158 | 1.290 |
| AD-1656634.1 | 68.804 | 4.037 | 72.522 | 8.828 |
| AD-1656622.1 | 64.773 | 3.958 | 61.094 | 3.211 |
| AD-1656602.1 | 90.930 | 8.206 | 97.503 | 6.469 |
| AD-1656591.1 | 67.929 | 6.205 | 50.621 | 2.250 |
| AD-1656587.1 | 130.517 | 14.507 | 112.466 | 5.072 |
| AD-1656570.1 | 54.831 | 8.259 | 67.275 | 4.341 |
| AD-1656559.1 | 99.324 | 16.587 | 94.525 | 10.986 |
| AD-1656547.1 | 47.881 | 6.431 | 47.546 | 13.507 |
| AD-1656535.1 | 45.730 | 4.759 | 47.345 | 3.749 |
| AD-1656504.1 | 18.728 | 1.669 | 28.522 | 5.548 |
| AD-1656492.1 | 56.190 | 5.381 | 90.874 | 4.381 |
| AD-1656480.1 | 123.739 | 13.306 | 109.755 | 2.612 |
| AD-1656468.1 | 109.278 | 12.715 | 110.494 | 6.230 |
| AD-1656449.1 | 59.340 | 4.424 | 63.755 | 3.688 |
| AD-1656437.1 | 37.011 | 10.928 | 37.870 | 1.699 |
| AD-1656417.1 | 71.490 | 5.406 | 76.482 | 4.881 |
| AD-1656406.1 | 24.283 | 4.442 | 22.173 | 1.760 |
| AD-1656392.1 | 32.989 | 8.824 | 25.987 | 9.238 |
| AD-1656383.1 | 136.848 | 18.447 | 130.314 | 12.433 |
| AD-1656372.1 | 108.305 | 5.297 | 103.207 | 3.375 |
| AD-1656360.1 | 67.214 | 8.162 | 87.294 | 8.571 |
| AD-1656333.1 | 28.433 | 5.041 | 26.718 | 2.141 |
| AD-1656319.1 | 56.184 | 4.092 | 52.184 | 3.756 |
| AD-1656307.1 | 102.315 | 8.887 | 96.572 | 4.261 |
| AD-1656292.1 | 26.799 | 5.415 | 30.140 | 4.062 |
| AD-1656280.1 | 42.240 | 7.706 | 36.464 | 2.246 |
| AD-1656265.1 | 72.020 | 14.577 | 80.758 | 6.589 |
| AD-1656260.1 | 102.537 | 13.790 | 115.141 | 23.155 |
| AD-1656254.1 | 114.062 | 4.103 | 126.823 | 2.379 |
| AD-1656233.1 | 99.276 | 14.267 | 93.223 | 3.260 |
| AD-1656220.1 | 52.087 | 6.444 | 47.137 | 2.748 |
| AD-1656196.1 | 69.556 | 7.457 | 78.281 | 3.008 |
| AD-1656185.1 | 59.059 | 6.916 | 59.771 | 1.753 |
| AD-1656164.1 | 52.037 | 5.249 | 49.894 | 1.671 |
| AD-1656146.1 | 61.416 | 9.782 | 69.569 | 10.125 |
| AD-1656139.1 | 81.533 | 5.159 | 79.120 | 3.969 |
| AD-1656125.1 | 106.018 | 7.449 | 120.205 | 7.924 |
| AD-1656108.1 | 92.474 | 5.568 | 120.457 | 12.135 |
| AD-1656097.1 | 86.405 | 6.346 | 95.682 | 6.379 |
| AD-1656086.1 | 97.906 | 14.028 | 98.562 | 6.521 |
| AD-1656074.1 | 78.172 | 13.741 | 91.731 | 5.480 |
| AD-1656054.1 | 85.044 | 8.142 | 90.847 | 4.543 |
| AD-1656043.1 | 50.700 | 6.709 | 47.910 | 3.196 |
| AD-1656026.1 | 109.741 | 12.514 | 93.624 | 7.670 |
| AD-1656008.1 | 86.662 | 8.226 | 98.543 | 23.583 |

### Example 4. Design, Synthesis and In Vitro Screening of Additional dsRNA Duplexes

Additional siRNAs were designed, synthesized, and prepared using methods known in the art and described above in Example 2.

A detailed list of the additional unmodified INHBE sense and antisense strand nucleotide sequences is shown in Table 19. A detailed list of the modified INHBE sense and antisense strand nucleotide sequences is shown in Table 20.

Single dose screens of the additional agents were performed by free uptake and transfection.

For free uptake, experiments were performed by adding 2.5 μl of siRNA duplexes in PBS per well into a 96 well plate. Complete growth media (47.5 μl) containing about 1.5×10⁴ primary human hepatocytes (PHH) or primary cynomolgus hepatocytes (PCH) were then added to the siRNA. Cells were incubated for 48 hours prior to RNA purification and RT-qPCR. Single dose experiments were performed at 250 nM, 100 nM, 10 nM and 1 nM final duplex concentration.

For transfections, cells (i.e., Hep3b cells, primary human hepatocytes, or primary cynomolgus hepatocytes) were grown to near confluence at 37° C. in an atmosphere of 5% CO₂ in Eagle's Minimum Essential Medium (Gibco) supplemented with 10% FBS (ATCC) before being released from the plate by trypsinization. Transfection was carried out by adding 7.5 μl of Opti-MEM plus 0.1 μl of Lipofectamine RNAiMax per well (Invitrogen, Carlsbad CA. cat 13778-150) to 2.5 μl of each siRNA duplex to an individual well in a 384-well plate. The mixture was then incubated at room temperature for 15 minutes. Forty μl of complete growth media without antibiotic containing ˜1.5×10⁴ cells were then added to the siRNA mixture. Cells were incubated for 24 hours prior to RNA purification. Single dose experiments were performed at 10, 1 and 0.1 nM final duplex concentration.

Total RNA isolation was performed using DYNABEADS. Briefly, cells were lysed in 10 μl of Lysis/Binding Buffer containing 3 μL of beads per well and mixed for 10 minutes on an electrostatic shaker. The washing steps were automated on a Biotek EL406, using a magnetic plate support. Beads were washed (in 3 μl) once in Buffer A, once in Buffer B, and twice in Buffer E, with aspiration steps in between. Following a final aspiration, complete 12 μl RT mixture was added to each well, as described below.

For cDNA synthesis, a master mix of 1.5 μl 10× Buffer, 0.6 μl 10×dNTPs, 1.5 μl Random primers, 0.75 μl Reverse Transcriptase, 0.75 μl RNase inhibitor and 9.9 μl of H2O per reaction were added per well. Plates were sealed, agitated for 10 minutes on an electrostatic shaker, and then incubated at 37 degrees C. for 2 hours. Following this, the plates were agitated at 80 degrees C. for 8 minutes.

RT-qPCR was performed as described above and relative fold change was calculated as described above.

The results of the transfection assays of the dsRNA agents listed in Tables 19 and 20 in Hep3b cells are shown in Table 21A.

The results of the free uptake experiments and the transfection assays of the dsRNA agents listed in Tables 19 and 20 in primary human hepatocytes (PHH) are shown in Table 21B.

The results of the free uptake experiments and the transfection assays of the dsRNA agents listed in Tables 19 and 20 in primary cynomolgus hepatocytes (PCH) are shown in Table 21C.

TABLE 19
Unmodified Sense and Antisense Strand Sequences of INHBE dsRNA Agents

| | | SEQ | Range in | | SEQ | Range in |
|---|---|---|---|---|---|---|
| | Sense Strand Sequence | ID | NM_ | Antisense Strand Sequence | ID | NM_ |
| Duplex Name | 5′ to 3′ | NO. | 031479.5 | 5′ to 3′ | NO. | 031479.5 |
| AD-1706265.1 | GCCAGACAUGAGCUGUGAGGU | 5525 | 5-25 | ACCUCACAGCUCAUGUCUGGCUA | 5978 | 3-25 |
| AD-1706266.1 | CCAGACAUGAGCUGUGAGGGU | 5526 | 6-26 | ACCCTCACAGCTCAUGUCUGGCU | 5979 | 4-26 |
| AD-1706268.1 | AGACAUGAGCUGUGAGGGUCU | 5527 | 8-28 | AGACCCTCACAGCUCAUGUCUGG | 5980 | 6-28 |
| AD-1706269.1 | GACAUGAGCUGUGAGGGUCAU | 5528 | 9-29 | ATGACCCUCACAGCUCAUGUCUG | 5981 | 7-29 |
| AD-1706270.1 | ACAUGAGCUGUGAGGGUCAAU | 5529 | 10-30 | ATUGACCCUCACAGCUCAUGUCU | 5982 | 8-30 |
| AD-1706271.1 | CAUGAGCUGUGAGGGUCAAGU | 5530 | 11-31 | ACUUGACCCUCACAGCUCAUGUC | 5983 | 9-31 |
| AD-1706272.1 | AUGAGCUGUGAGGGUCAAGCU | 5531 | 12-32 | AGCUTGACCCUCACAGCUCAUGU | 5984 | 10-32 |
| AD-1706273.1 | UGAGCUGUGAGGGUCAAGCAU | 5532 | 13-33 | ATGCTUGACCCTCACAGCUCAUG | 5985 | 11-33 |
| AD-1706274.1 | GAGCUGUGAGGGUCAAGCACU | 5533 | 14-34 | AGUGCUTGACCCUCACAGCUCAU | 5986 | 12-34 |
| AD-1706275.1 | AGCUGUGAGGGUCAAGCACAU | 5534 | 15-35 | ATGUGCTUGACCCUCACAGCUCA | 5987 | 13-35 |
| AD-1706276.1 | GCUGUGAGGGUCAAGCACAGU | 5535 | 16-36 | ACUGTGCUUGACCCUCACAGCUC | 5988 | 14-36 |
| AD-1706277.1 | CUGUGAGGGUCAAGCACAGCU | 5536 | 17-37 | AGCUGUGCUUGACCCUCACAGCU | 5989 | 15-37 |
| AD-1706278.1 | UGUGAGGGUCAAGCACAGCUU | 5537 | 18-38 | AAGCTGTGCUUGACCCUCACAGC | 5990 | 16-38 |
| AD-1706279.1 | GUGAGGGUCAAGCACAGCUAU | 5538 | 19-39 | ATAGCUGUGCUTGACCCUCACAG | 5991 | 17-39 |
| AD-1706280.1 | UGAGGGUCAAGCACAGCUAUU | 5539 | 20-40 | AAUAGCTGUGCTUGACCCUCACA | 5992 | 18-40 |
| AD-1706281.1 | GAGGGUCAAGCACAGCUAUCU | 129 | 21-41 | AGAUAGCUGUGCUUGACCCUCAC | 263 | 19-41 |
| AD-1706282.1 | AGGGUCAAGCACAGCUAUCCU | 5540 | 22-42 | AGGATAGCUGUGCUUGACCCUCA | 5993 | 20-42 |
| AD-1706283.1 | GGGUCAAGCACAGCUAUCCAU | 5541 | 23-43 | ATGGAUAGCUGTGCUUGACCCUC | 5994 | 21-43 |
| AD-1706287.1 | CAAGCACAGCUAUCCAUCAGU | 5542 | 27-47 | ACUGAUGGAUAGCUGUGCUUGAC | 5995 | 25-47 |
| AD-1706288.1 | AAGCACAGCUAUCCAUCAGAU | 5543 | 28-48 | ATCUGATGGAUAGCUGUGCUUGA | 5996 | 26-48 |
| AD-1706290.1 | GCACAGCUAUCCAUCAGAUGU | 5544 | 30-50 | ACAUCUGAUGGAUAGCUGUGCUU | 5997 | 28-50 |
| AD-1706291.1 | CACAGCUAUCCAUCAGAUGAU | 5545 | 31-51 | ATCATCTGAUGGAUAGCUGUGCU | 5998 | 29-51 |
| AD-1706292.1 | ACAGCUAUCCAUCAGAUGAUU | 5546 | 32-52 | AAUCAUCUGAUGGAUAGCUGUGC | 5999 | 30-52 |
| AD-1706293.1 | CAGCUAUCCAUCAGAUGAUCU | 5547 | 33-53 | AGAUCATCUGATGGAUAGCUGUG | 6000 | 31-53 |
| AD-1706294.1 | AGCUAUCCAUCAGAUGAUCUU | 5548 | 34-54 | AAGATCAUCUGAUGGAUAGCUGU | 6001 | 32-54 |
| AD-1706295.1 | GCUAUCCAUCAGAUGAUCUAU | 5549 | 35-55 | ATAGAUCAUCUGAUGGAUAGCUG | 6002 | 33-55 |
| AD-1706296.1 | CUAUCCAUCAGAUGAUCUACU | 5550 | 36-56 | AGUAGATCAUCTGAUGGAUAGCU | 6003 | 34-56 |
| AD-1706297.1 | UAUCCAUCAGAUGAUCUACUU | 5551 | 37-57 | AAGUAGAUCAUCUGAUGGAUAGC | 6004 | 35-57 |
| AD-1706298.1 | AUCCAUCAGAUGAUCUACUUU | 130 | 38-58 | AAAGTAGAUCATCUGAUGGAUAG | 6005 | 36-58 |
| AD-1706299.1 | UCCAUCAGAUGAUCUACUUUU | 5552 | 39-59 | AAAAGUAGAUCAUCUGAUGGAUA | 6006 | 37-59 |
| AD-1706300.1 | CCAUCAGAUGAUCUACUUUCU | 5553 | 40-60 | AGAAAGTAGAUCAUCUGAUGGAU | 6007 | 38-60 |
| AD-1706301.1 | CAUCAGAUGAUCUACUUUCAU | 5554 | 41-61 | ATGAAAGUAGATCAUCUGAUGGA | 6008 | 39-61 |
| AD-1706302.1 | AUCAGAUGAUCUACUUUCAGU | 5555 | 42-62 | ACUGAAAGUAGAUCAUCUGAUGG | 6009 | 40-62 |
| AD-1706303.1 | UCAGAUGAUCUACUUUCAGCU | 5556 | 43-63 | AGCUGAAAGUAGAUCAUCUGAUG | 6010 | 41-63 |
| AD-1706304.1 | CAGAUGAUCUACUUUCAGCCU | 5557 | 44-64 | AGGCTGAAAGUAGAUCAUCUGAU | 6011 | 42-64 |
| AD-1706305.1 | AGAUGAUCUACUUUCAGCCUU | 5558 | 45-65 | AAGGCUGAAAGTAGAUCAUCUGA | 6012 | 43-65 |
| AD-1706306.1 | GAUGAUCUACUUUCAGCCUUU | 5559 | 46-66 | AAAGGCTGAAAGUAGAUCAUCUG | 6013 | 44-66 |
| AD-1706307.1 | AUGAUCUACUUUCAGCCUUCU | 5560 | 47-67 | AGAAGGCUGAAAGUAGAUCAUCU | 6014 | 45-67 |
| AD-1706308.1 | UGAUCUACUUUCAGCCUUCCU | 5561 | 48-68 | AGGAAGGCUGAAAGUAGAUCAUC | 6015 | 46-68 |
| AD-1706309.1 | GAUCUACUUUCAGCCUUCCUU | 131 | 49-69 | AAGGAAGGCUGAAAGUAGAUCAU | 265 | 47-69 |
| AD-1706310.1 | AUCUACUUUCAGCCUUCCUGU | 5562 | 50-70 | ACAGGAAGGCUGAAAGUAGAUCA | 6016 | 48-70 |
| AD-1706311.1 | UCUACUUUCAGCCUUCCUGAU | 5563 | 51-71 | ATCAGGAAGGCTGAAAGUAGAUC | 6017 | 49-71 |
| AD-1706312.1 | CUACUUUCAGCCUUCCUGAGU | 5564 | 52-72 | ACUCAGGAAGGCUGAAAGUAGAU | 6018 | 50-72 |
| AD-1706313.1 | UACUUUCAGCCUUCCUGAGUU | 5565 | 53-73 | AACUCAGGAAGGCUGAAAGUAGA | 6019 | 51-73 |
| AD-1706314.1 | ACUUUCAGCCUUCCUGAGUCU | 5566 | 54-74 | AGACTCAGGAAGGCUGAAAGUAG | 6020 | 52-74 |
| AD-1706318.1 | UCAGCCUUCCUGAGUCCCAGU | 5567 | 58-78 | ACUGGGACUCAGGAAGGCUGAAA | 6021 | 56-78 |
| AD-1706337.1 | GACAAUAGAAGACAGGUGGCU | 5568 | 77-97 | AGCCACCUGUCTUCUAUUGUCUG | 6022 | 75-97 |
| AD-1706338.1 | ACAAUAGAAGACAGGUGGCUU | 5569 | 78-98 | AAGCCACCUGUCUUCUAUUGUCU | 6023 | 76-98 |
| AD-1706380.1 | GUGGCAGUGGUGUCUGCUGUU | 136 | 120-140 | AACAGCAGACACCACUGCCACAC | 270 | 118-140 |
| AD-1706381.1 | UGGCAGUGGUGUCUGCUGUCU | 5570 | 121-141 | AGACAGCAGACACCACUGCCACA | 6024 | 119-141 |
| AD-1706382.1 | GGCAGUGGUGUCUGCUGUCAU | 5571 | 122-142 | ATGACAGCAGACACCACUGCCAC | 6025 | 120-142 |
| AD-1706383.1 | GCAGUGGUGUCUGCUGUCACU | 5572 | 123-143 | AGUGACAGCAGACACCACUGCCA | 6026 | 121-143 |
| AD-1706384.1 | CAGUGGUGUCUGCUGUCACUU | 5573 | 124-144 | AAGUGACAGCAGACACCACUGCC | 6027 | 122-144 |
| AD-1706385.1 | AGUGGUGUCUGCUGUCACUGU | 5574 | 125-145 | ACAGTGACAGCAGACACCACUGC | 6028 | 123-145 |
| AD-1706386.1 | GUGGUGUCUGCUGUCACUGUU | 5575 | 126-146 | AACAGUGACAGCAGACACCACUG | 6029 | 124-146 |
| AD-1706387.1 | UGGUGUCUGCUGUCACUGUGU | 5576 | 127-147 | ACACAGTGACAGCAGACACCACU | 6030 | 125-147 |
| AD-1706388.1 | GGUGUCUGCUGUCACUGUGCU | 5577 | 128-148 | AGCACAGUGACAGCAGACACCAC | 6031 | 126-148 |
| AD-1706407.1 | CAGACUCAACAGACGGAGCAU | 5578 | 168-188 | ATGCTCCGUCUGUUGAGUCUGAU | 6032 | 166-188 |
| AD-1706408.1 | AGACUCAACAGACGGAGCAAU | 5579 | 169-189 | ATUGCUCCGUCTGUUGAGUCUGA | 6033 | 167-189 |
| AD-1706409.1 | GACUCAACAGACGGAGCAACU | 5580 | 170-190 | AGUUGCTCCGUCUGUUGAGUCUG | 6034 | 168-190 |
| AD-1706410.1 | ACUCAACAGACGGAGCAACUU | 5581 | 171-191 | AAGUTGCUCCGTCUGUUGAGUCU | 6035 | 169-191 |
| AD-1706411.1 | CUCAACAGACGGAGCAACUGU | 5582 | 172-192 | ACAGTUGCUCCGUCUGUUGAGUC | 6036 | 170-192 |
| AD-1706412.1 | UCAACAGACGGAGCAACUGCU | 5583 | 173-193 | AGCAGUTGCUCCGUCUGUUGAGU | 6037 | 171-193 |
| AD-1706413.1 | CAACAGACGGAGCAACUGCCU | 5584 | 174-194 | AGGCAGTUGCUCCGUCUGUUGAG | 6038 | 172-194 |
| AD-1706414.1 | AACAGACGGAGCAACUGCCAU | 5585 | 175-195 | ATGGCAGUUGCTCCGUCUGUUGA | 6039 | 173-195 |
| AD-1706415.1 | ACAGACGGAGCAACUGCCAUU | 5586 | 176-196 | AAUGGCAGUUGCUCCGUCUGUUG | 6040 | 174-196 |
| AD-1706416.1 | CAGACGGAGCAACUGCCAUCU | 5587 | 177-197 | AGAUGGCAGUUGCUCCGUCUGUU | 6041 | 175-197 |
| AD-1706417.1 | AGACGGAGCAACUGCCAUCCU | 5588 | 178-198 | AGGATGGCAGUTGCUCCGUCUGU | 6042 | 176-198 |
| AD-1706418.1 | GACGGAGCAACUGCCAUCCGU | 5589 | 179-199 | ACGGAUGGCAGTUGCUCCGUCUG | 6043 | 177-199 |
| AD-1706419.1 | ACGGAGCAACUGCCAUCCGAU | 139 | 180-200 | ATCGGATGGCAGUUGCUCCGUCU | 6044 | 178-200 |
| AD-1706420.1 | CGGAGCAACUGCCAUCCGAGU | 5590 | 181-201 | ACUCGGAUGGCAGUUGCUCCGUC | 6045 | 179-201 |
| AD-1706421.1 | GGAGCAACUGCCAUCCGAGGU | 5591 | 182-202 | ACCUCGGAUGGCAGUUGCUCCGU | 6046 | 180-202 |
| AD-1706423.1 | AGCAACUGCCAUCCGAGGCUU | 5592 | 184-204 | AAGCCUCGGAUGGCAGUUGCUCC | 6047 | 182-204 |
| AD-1706424.1 | GCAACUGCCAUCCGAGGCUCU | 5593 | 185-205 | AGAGCCTCGGATGGCAGUUGCUC | 6048 | 183-205 |
| AD-1706425.1 | CAACUGCCAUCCGAGGCUCCU | 5594 | 186-206 | AGGAGCCUCGGAUGGCAGUUGCU | 6049 | 184-206 |
| AD-1706520.1 | CAAGCAGAACGAGCUCUGGUU | 146 | 339-359 | AACCAGAGCUCGUUCUGCUUGGG | 280 | 337-359 |
| AD-1706521.1 | AAGCAGAACGAGCUCUGGUGU | 5595 | 340-360 | ACACCAGAGCUCGUUCUGCUUGG | 6050 | 338-360 |
| AD-1706522.1 | AGCAGAACGAGCUCUGGUGCU | 5596 | 341-361 | AGCACCAGAGCTCGUUCUGCUUG | 6051 | 339-361 |
| AD-1706523.1 | GCAGAACGAGCUCUGGUGCUU | 5597 | 342-362 | AAGCACCAGAGCUCGUUCUGCUU | 6052 | 340-362 |
| AD-1706524.1 | CAGAACGAGCUCUGGUGCUGU | 5598 | 343-363 | ACAGCACCAGAGCUCGUUCUGCU | 6053 | 341-363 |
| AD-1706525.1 | AGAACGAGCUCUGGUGCUGGU | 5599 | 344-364 | ACCAGCACCAGAGCUCGUUCUGC | 6054 | 342-364 |
| AD-1706526.1 | GAACGAGCUCUGGUGCUGGAU | 5600 | 345-365 | ATCCAGCACCAGAGCUCGUUCUG | 6055 | 343-365 |
| AD-1706527.1 | AACGAGCUCUGGUGCUGGAGU | 5601 | 346-366 | ACUCCAGCACCAGAGCUCGUUCU | 6056 | 344-366 |
| AD-1706528.1 | ACGAGCUCUGGUGCUGGAGCU | 5602 | 347-367 | AGCUCCAGCACCAGAGCUCGUUC | 6057 | 345-367 |
| AD-1706530.1 | GAGCUCUGGUGCUGGAGCUAU | 5603 | 349-369 | ATAGCUCCAGCACCAGAGCUCGU | 6058 | 347-369 |
| AD-1706532.1 | GCUCUGGUGCUGGAGCUAGCU | 5604 | 351-371 | AGCUAGCUCCAGCACCAGAGCUC | 6059 | 349-371 |
| AD-1706533.1 | CUCUGGUGCUGGAGCUAGCCU | 5605 | 352-372 | AGGCTAGCUCCAGCACCAGAGCU | 6060 | 350-372 |
| AD-1706534.1 | UCUGGUGCUGGAGCUAGCCAU | 5606 | 353-373 | ATGGCUAGCUCCAGCACCAGAGC | 6061 | 351-373 |
| AD-1706535.1 | CUGGUGCUGGAGCUAGCCAAU | 147 | 354-374 | ATUGGCTAGCUCCAGCACCAGAG | 6062 | 352-374 |
| AD-1706536.1 | UGGUGCUGGAGCUAGCCAAGU | 5607 | 355-375 | ACUUGGCUAGCTCCAGCACCAGA | 6063 | 353-375 |
| AD-1706537.1 | GGUGCUGGAGCUAGCCAAGCU | 5608 | 356-376 | AGCUTGGCUAGCUCCAGCACCAG | 6064 | 354-376 |
| AD-1706538.1 | GUGCUGGAGCUAGCCAAGCAU | 5609 | 357-377 | ATGCTUGGCUAGCUCCAGCACCA | 6065 | 355-377 |
| AD-1706539.1 | UGCUGGAGCUAGCCAAGCAGU | 5610 | 358-378 | ACUGCUTGGCUAGCUCCAGCACC | 6066 | 356-378 |
| AD-1706543.1 | GGAGCUAGCCAAGCAGCAAAU | 5611 | 362-382 | ATUUGCTGCUUGGCUAGCUCCAG | 6067 | 360-382 |
| AD-1706544.1 | GAGCUAGCCAAGCAGCAAAUU | 5612 | 363-383 | AAUUTGCUGCUTGGCUAGCUCCA | 6068 | 361-383 |
| AD-1706545.1 | AGCUAGCCAAGCAGCAAAUCU | 5613 | 364-384 | AGAUTUGCUGCTUGGCUAGCUCC | 6069 | 362-384 |
| AD-1706546.1 | GCUAGCCAAGCAGCAAAUCCU | 5614 | 365-385 | AGGATUTGCUGCUUGGCUAGCUC | 6070 | 363-385 |
| AD-1706547.1 | CUAGCCAAGCAGCAAAUCCUU | 148 | 366-386 | AAGGAUTUGCUGCUUGGCUAGCU | 6071 | 364-386 |
| AD-1706548.1 | UAGCCAAGCAGCAAAUCCUGU | 5615 | 367-387 | ACAGGATUUGCTGCUUGGCUAGC | 6072 | 365-387 |
| AD-1706549.1 | AGCCAAGCAGCAAAUCCUGGU | 5616 | 368-388 | ACCAGGAUUUGCUGCUUGGCUAG | 6073 | 366-388 |
| AD-1706550.1 | GCCAAGCAGCAAAUCCUGGAU | 5617 | 369-389 | ATCCAGGAUUUGCUGCUUGGCUA | 6074 | 367-389 |
| AD-1706578.1 | ACCUGACCAGUCGUCCCAGAU | 5618 | 397-417 | ATCUGGGACGACUGGUCAGGUGC | 6075 | 395-417 |
| AD-1706579.1 | CCUGACCAGUCGUCCCAGAAU | 5619 | 398-418 | ATUCTGGGACGACUGGUCAGGUG | 6076 | 396-418 |
| AD-1706580.1 | CUGACCAGUCGUCCCAGAAUU | 5620 | 399-419 | AAUUCUGGGACGACUGGUCAGGU | 6077 | 397-419 |
| AD-1706581.1 | UGACCAGUCGUCCCAGAAUAU | 5621 | 400-420 | ATAUTCTGGGACGACUGGUCAGG | 6078 | 398-420 |
| AD-1706582.1 | GACCAGUCGUCCCAGAAUAAU | 5622 | 401-421 | ATUATUCUGGGACGACUGGUCAG | 6079 | 399-421 |
| AD-1706583.1 | ACCAGUCGUCCCAGAAUAACU | 66 | 402-422 | AGUUAUTCUGGGACGACUGGUCA | 57 | 400-422 |
| AD-1706584.1 | CCAGUCGUCCCAGAAUAACUU | 5623 | 403-423 | AAGUTATUCUGGGACGACUGGUC | 6080 | 401-423 |
| AD-1706585.1 | CAGUCGUCCCAGAAUAACUCU | 5624 | 404-424 | AGAGTUAUUCUGGGACGACUGGU | 6081 | 402-424 |
| AD-1706586.1 | AGUCGUCCCAGAAUAACUCAU | 5625 | 405-425 | ATGAGUTAUUCTGGGACGACUGG | 6082 | 403-425 |
| AD-1706587.1 | GUCGUCCCAGAAUAACUCAUU | 5626 | 406-426 | AAUGAGTUAUUCUGGGACGACUG | 6083 | 404-426 |
| AD-1706588.1 | UCGUCCCAGAAUAACUCAUCU | 151 | 407-427 | AGAUGAGUUAUTCUGGGACGACU | 6084 | 405-427 |
| AD-1706589.1 | CGUCCCAGAAUAACUCAUCCU | 5627 | 408-428 | AGGATGAGUUATUCUGGGACGAC | 6085 | 406-428 |
| AD-1706590.1 | GUCCCAGAAUAACUCAUCCUU | 5628 | 409-429 | AAGGAUGAGUUAUUCUGGGACGA | 6086 | 407-429 |
| AD-1706591.1 | UCCCAGAAUAACUCAUCCUCU | 5629 | 410-430 | AGAGGATGAGUTAUUCUGGGACG | 6087 | 408-430 |
| AD-1706592.1 | CCCAGAAUAACUCAUCCUCCU | 5630 | 411-431 | AGGAGGAUGAGTUAUUCUGGGAC | 6088 | 409-431 |
| AD-1706593.1 | CCAGAAUAACUCAUCCUCCAU | 67 | 412-432 | ATGGAGGAUGAGUUAUUCUGGGA | 59 | 410-432 |
| AD-1706594.1 | CAGAAUAACUCAUCCUCCACU | 5631 | 413-433 | AGUGGAGGAUGAGUUAUUCUGGG | 6089 | 411-433 |
| AD-1706635.1 | GGGAGUGUGGCUCCAGGGAAU | 5632 | 474-494 | ATUCCCTGGAGCCACACUCCCUG | 6090 | 472-494 |
| AD-1706636.1 | GGAGUGUGGCUCCAGGGAAUU | 5633 | 475-495 | AAUUCCCUGGAGCCACACUCCCU | 6091 | 473-495 |
| AD-1706637.1 | GAGUGUGGCUCCAGGGAAUGU | 5634 | 476-496 | ACAUTCCCUGGAGCCACACUCCC | 6092 | 474-496 |
| AD-1706638.1 | AGUGUGGCUCCAGGGAAUGGU | 154 | 477-497 | ACCATUCCCUGGAGCCACACUCC | 6093 | 475-497 |
| AD-1706639.1 | GAGGAGGUCAUCAGCUUUGCU | 5635 | 498-518 | AGCAAAGCUGATGACCUCCUCCC | 6094 | 496-518 |
| AD-1706640.1 | AGGAGGUCAUCAGCUUUGCUU | 5636 | 499-519 | AAGCAAAGCUGAUGACCUCCUCC | 6095 | 497-519 |
| AD-1706641.1 | GGAGGUCAUCAGCUUUGCUAU | 5637 | 500-520 | ATAGCAAAGCUGAUGACCUCCUC | 6096 | 498-520 |
| AD-1706642.1 | GAGGUCAUCAGCUUUGCUACU | 5638 | 501-521 | AGUAGCAAAGCTGAUGACCUCCU | 6097 | 499-521 |
| AD-1706643.1 | AGGUCAUCAGCUUUGCUACUU | 5639 | 502-522 | AAGUAGCAAAGCUGAUGACCUCC | 6098 | 500-522 |
| AD-1706644.1 | GGUCAUCAGCUUUGCUACUGU | 5640 | 503-523 | ACAGTAGCAAAGCUGAUGACCUC | 6099 | 501-523 |
| AD-1706645.1 | GUCAUCAGCUUUGCUACUGUU | 5641 | 504-524 | AACAGUAGCAAAGCUGAUGACCU | 6100 | 502-524 |
| AD-1706646.1 | UCAUCAGCUUUGCUACUGUCU | 5642 | 505-525 | AGACAGTAGCAAAGCUGAUGACC | 6101 | 503-525 |
| AD-1706649.1 | UCAGCUUUGCUACUGUCACAU | 5643 | 508-528 | ATGUGACAGUAGCAAAGCUGAUG | 6102 | 506-528 |
| AD-1706650.1 | CAGCUUUGCUACUGUCACAGU | 5644 | 509-529 | ACUGTGACAGUAGCAAAGCUGAU | 6103 | 507-529 |
| AD-1706651.1 | AGCUUUGCUACUGUCACAGAU | 5645 | 510-530 | ATCUGUGACAGTAGCAAAGCUGA | 6104 | 508-530 |
| AD-1706652.1 | GCUUUGCUACUGUCACAGACU | 5646 | 511-531 | AGUCTGTGACAGUAGCAAAGCUG | 6105 | 509-531 |
| AD-1706653.1 | CUUUGCUACUGUCACAGACUU | 5647 | 512-532 | AAGUCUGUGACAGUAGCAAAGCU | 6106 | 510-532 |
| AD-1706654.1 | UUUGCUACUGUCACAGACUCU | 5648 | 513-533 | AGAGTCTGUGACAGUAGCAAAGC | 6107 | 511-533 |
| AD-1706655.1 | UUGCUACUGUCACAGACUCCU | 5649 | 514-534 | AGGAGUCUGUGACAGUAGCAAAG | 6108 | 512-534 |
| AD-1706656.1 | UGCUACUGUCACAGACUCCAU | 5650 | 515-535 | ATGGAGTCUGUGACAGUAGCAAA | 6109 | 513-535 |
| AD-1706660.1 | ACUGUCACAGACUCCACUUCU | 5651 | 519-539 | AGAAGUGGAGUCUGUGACAGUAG | 6110 | 517-539 |
| AD-1706661.1 | CUGUCACAGACUCCACUUCAU | 68 | 520-540 | ATGAAGTGGAGTCUGUGACAGUA | 6111 | 518-540 |
| AD-1706662.1 | UGUCACAGACUCCACUUCAGU | 69 | 521-541 | ACUGAAGUGGAGUCUGUGACAGU | 61 | 519-541 |
| AD-1706664.1 | UCACAGACUCCACUUCAGCCU | 5652 | 523-543 | AGGCTGAAGUGGAGUCUGUGACA | 6112 | 521-543 |
| AD-1706665.1 | CACAGACUCCACUUCAGCCUU | 5653 | 524-544 | AAGGCUGAAGUGGAGUCUGUGAC | 6113 | 522-544 |
| AD-1706666.1 | ACAGACUCCACUUCAGCCUAU | 5654 | 525-545 | ATAGGCTGAAGTGGAGUCUGUGA | 6114 | 523-545 |
| AD-1706667.1 | CAGACUCCACUUCAGCCUACU | 5655 | 526-546 | AGUAGGCUGAAGUGGAGUCUGUG | 6115 | 524-546 |
| AD-1706668.1 | AGACUCCACUUCAGCCUACAU | 5656 | 527-547 | ATGUAGGCUGAAGUGGAGUCUGU | 6116 | 525-547 |
| AD-1706669.1 | GACUCCACUUCAGCCUACAGU | 5657 | 528-548 | ACUGTAGGCUGAAGUGGAGUCUG | 6117 | 526-548 |
| AD-1706670.1 | ACUCCACUUCAGCCUACAGCU | 5658 | 529-549 | AGCUGUAGGCUGAAGUGGAGUCU | 6118 | 527-549 |
| AD-1706671.1 | CUCCACUUCAGCCUACAGCUU | 5659 | 530-550 | AAGCTGTAGGCTGAAGUGGAGUC | 6119 | 528-550 |
| AD-1706672.1 | UCCACUUCAGCCUACAGCUCU | 156 | 531-551 | AGAGCUGUAGGCUGAAGUGGAGU | 291 | 529-551 |
| AD-1706673.1 | CCACUUCAGCCUACAGCUCCU | 5660 | 532-552 | AGGAGCTGUAGGCUGAAGUGGAG | 6120 | 530-552 |
| AD-1706674.1 | CACUUCAGCCUACAGCUCCCU | 5661 | 533-553 | AGGGAGCUGUAGGCUGAAGUGGA | 6121 | 531-553 |
| AD-1706675.1 | ACUUCAGCCUACAGCUCCCUU | 5662 | 534-554 | AAGGGAGCUGUAGGCUGAAGUGG | 6122 | 532-554 |
| AD-1706678.1 | UCAGCCUACAGCUCCCUGCUU | 5663 | 537-557 | AAGCAGGGAGCTGUAGGCUGAAG | 6123 | 535-557 |
| AD-1706729.1 | CACCUGUACCAUGCCCGCCUU | 5664 | 588-608 | AAGGCGGGCAUGGUACAGGUGGU | 6124 | 586-608 |
| AD-1706731.1 | CCUGUACCAUGCCCGCCUGUU | 5665 | 590-610 | AACAGGCGGGCAUGGUACAGGUG | 6125 | 588-610 |
| AD-1706732.1 | CUGUACCAUGCCCGCCUGUGU | 5666 | 591-611 | ACACAGGCGGGCAUGGUACAGGU | 6126 | 589-611 |
| AD-1706735.1 | UACCAUGCCCGCCUGUGGCUU | 160 | 594-614 | AAGCCACAGGCGGGCAUGGUACA | 295 | 592-614 |
| AD-1706745.1 | CACCCUUCCUGGCACUCUUUU | 5667 | 626-646 | AAAAGAGUGCCAGGAAGGGUGGG | 6127 | 624-646 |
| AD-1706746.1 | ACCCUUCCUGGCACUCUUUGU | 5668 | 627-647 | ACAAAGAGUGCCAGGAAGGGUGG | 6128 | 625-647 |
| AD-1706747.1 | CCCUUCCUGGCACUCUUUGCU | 161 | 628-648 | AGCAAAGAGUGCCAGGAAGGGUG | 296 | 626-648 |
| AD-1706748.1 | CCUUCCUGGCACUCUUUGCUU | 5669 | 629-649 | AAGCAAAGAGUGCCAGGAAGGGU | 6129 | 627-649 |
| AD-1706749.1 | CUUCCUGGCACUCUUUGCUUU | 5670 | 630-650 | AAAGCAAAGAGTGCCAGGAAGGG | 6130 | 628-650 |
| AD-1706750.1 | UUCCUGGCACUCUUUGCUUGU | 5671 | 631-651 | ACAAGCAAAGAGUGCCAGGAAGG | 6131 | 629-651 |
| AD-1706751.1 | UCCUGGCACUCUUUGCUUGAU | 5672 | 632-652 | ATCAAGCAAAGAGUGCCAGGAAG | 6132 | 630-652 |
| AD-1706752.1 | CCUGGCACUCUUUGCUUGAGU | 5673 | 633-653 | ACUCAAGCAAAGAGUGCCAGGAA | 6133 | 631-653 |
| AD-1706753.1 | CUGGCACUCUUUGCUUGAGGU | 5674 | 634-654 | ACCUCAAGCAAAGAGUGCCAGGA | 6134 | 632-654 |
| AD-1706754.1 | UGGCACUCUUUGCUUGAGGAU | 5675 | 635-655 | ATCCTCAAGCAAAGAGUGCCAGG | 6135 | 633-655 |
| AD-1706755.1 | GGCACUCUUUGCUUGAGGAUU | 5676 | 636-656 | AAUCCUCAAGCAAAGAGUGCCAG | 6136 | 634-656 |
| AD-1706756.1 | GCACUCUUUGCUUGAGGAUCU | 5677 | 637-657 | AGAUCCTCAAGCAAAGAGUGCCA | 6137 | 635-657 |
| AD-1706758.1 | ACUCUUUGCUUGAGGAUCUUU | 5678 | 639-659 | AAAGAUCCUCAAGCAAAGAGUGC | 6138 | 637-659 |
| AD-1706759.1 | CUCUUUGCUUGAGGAUCUUCU | 162 | 640-660 | AGAAGATCCUCAAGCAAAGAGUG | 6139 | 638-660 |
| AD-1706760.1 | UCUUUGCUUGAGGAUCUUCCU | 5679 | 641-661 | AGGAAGAUCCUCAAGCAAAGAGU | 6140 | 639-661 |
| AD-1706761.1 | CUUUGCUUGAGGAUCUUCCGU | 70 | 642-662 | ACGGAAGAUCCTCAAGCAAAGAG | 62 | 640-662 |
| AD-1706762.1 | UUUGCUUGAGGAUCUUCCGAU | 5680 | 643-663 | ATCGGAAGAUCCUCAAGCAAAGA | 6141 | 641-663 |
| AD-1706763.1 | UUGCUUGAGGAUCUUCCGAUU | 5681 | 644-664 | AAUCGGAAGAUCCUCAAGCAAAG | 6142 | 642-664 |
| AD-1706764.1 | UGCUUGAGGAUCUUCCGAUGU | 5682 | 645-665 | ACAUCGGAAGATCCUCAAGCAAA | 6143 | 643-665 |
| AD-1706765.1 | GCUUGAGGAUCUUCCGAUGGU | 5683 | 646-666 | ACCATCGGAAGAUCCUCAAGCAA | 6144 | 644-666 |
| AD-1706796.1 | CUGGCUGAGCACCACAUCACU | 5684 | 702-722 | AGUGAUGUGGUGCUCAGCCAGGA | 6145 | 700-722 |
| AD-1706797.1 | UGGCUGAGCACCACAUCACCU | 5685 | 703-723 | AGGUGATGUGGTGCUCAGCCAGG | 6146 | 701-723 |
| AD-1706798.1 | GGCUGAGCACCACAUCACCAU | 5686 | 704-724 | ATGGTGAUGUGGUGCUCAGCCAG | 6147 | 702-724 |
| AD-1706799.1 | GCUGAGCACCACAUCACCAAU | 5687 | 705-725 | ATUGGUGAUGUGGUGCUCAGCCA | 6148 | 703-725 |
| AD-1706800.1 | CUGAGCACCACAUCACCAACU | 5688 | 706-726 | AGUUGGTGAUGTGGUGCUCAGCC | 6149 | 704-726 |
| AD-1706801.1 | UGAGCACCACAUCACCAACCU | 5689 | 707-727 | AGGUTGGUGAUGUGGUGCUCAGC | 6150 | 705-727 |
| AD-1706802.1 | GAGCACCACAUCACCAACCUU | 164 | 708-728 | AAGGTUGGUGATGUGGUGCUCAG | 6151 | 706-728 |
| AD-1706803.1 | AGCACCACAUCACCAACCUGU | 5690 | 709-729 | ACAGGUTGGUGAUGUGGUGCUCA | 6152 | 707-729 |
| AD-1706804.1 | GCACCACAUCACCAACCUGGU | 5691 | 710-730 | ACCAGGTUGGUGAUGUGGUGCUC | 6153 | 708-730 |
| AD-1706807.1 | CCACAUCACCAACCUGGGCUU | 5692 | 713-733 | AAGCCCAGGUUGGUGAUGUGGUG | 6154 | 711-733 |
| AD-1706808.1 | CACAUCACCAACCUGGGCUGU | 5693 | 714-734 | ACAGCCCAGGUTGGUGAUGUGGU | 6155 | 712-734 |
| AD-1706809.1 | ACAUCACCAACCUGGGCUGGU | 5694 | 715-735 | ACCAGCCCAGGTUGGUGAUGUGG | 6156 | 713-735 |
| AD-1706811.1 | AUCACCAACCUGGGCUGGCAU | 5695 | 717-737 | ATGCCAGCCCAGGUUGGUGAUGU | 6157 | 715-737 |
| AD-1706812.1 | UCACCAACCUGGGCUGGCAUU | 5696 | 718-738 | AAUGCCAGCCCAGGUUGGUGAUG | 6158 | 716-738 |
| AD-1706831.1 | UACCUUAACUCUGCCCUCUAU | 5697 | 737-757 | ATAGAGGGCAGAGUUAAGGUAUG | 6159 | 735-757 |
| AD-1706832.1 | ACCUUAACUCUGCCCUCUAGU | 5698 | 738-758 | ACUAGAGGGCAGAGUUAAGGUAU | 6160 | 736-758 |
| AD-1706833.1 | CCUUAACUCUGCCCUCUAGUU | 5699 | 739-759 | AACUAGAGGGCAGAGUUAAGGUA | 6161 | 737-759 |
| AD-1706834.1 | CUUAACUCUGCCCUCUAGUGU | 5700 | 740-760 | ACACTAGAGGGCAGAGUUAAGGU | 6162 | 738-760 |
| AD-1706835.1 | UUAACUCUGCCCUCUAGUGGU | 5701 | 741-761 | ACCACUAGAGGGCAGAGUUAAGG | 6163 | 739-761 |
| AD-1706836.1 | UAACUCUGCCCUCUAGUGGCU | 5702 | 742-762 | AGCCACTAGAGGGCAGAGUUAAG | 6164 | 740-762 |
| AD-1706848.1 | AAGUCUGGUGUCCUGAAACUU | 5703 | 774-794 | AAGUTUCAGGACACCAGACUUCU | 6165 | 772-794 |
| AD-1706849.1 | AGUCUGGUGUCCUGAAACUGU | 5704 | 775-795 | ACAGTUTCAGGACACCAGACUUC | 6166 | 773-795 |
| AD-1706850.1 | GUCUGGUGUCCUGAAACUGCU | 5705 | 776-796 | AGCAGUTUCAGGACACCAGACUU | 6167 | 774-796 |
| AD-1706851.1 | UCUGGUGUCCUGAAACUGCAU | 5706 | 777-797 | ATGCAGTUUCAGGACACCAGACU | 6168 | 775-797 |
| AD-1706852.1 | CUGGUGUCCUGAAACUGCAAU | 5707 | 778-798 | ATUGCAGUUUCAGGACACCAGAC | 6169 | 776-798 |
| AD-1706853.1 | UGGUGUCCUGAAACUGCAACU | 5708 | 779-799 | AGUUGCAGUUUCAGGACACCAGA | 6170 | 777-799 |
| AD-1706854.1 | GGUGUCCUGAAACUGCAACUU | 5709 | 780-800 | AAGUTGCAGUUTCAGGACACCAG | 6171 | 778-800 |
| AD-1706855.1 | GUGUCCUGAAACUGCAACUAU | 5710 | 781-801 | ATAGTUGCAGUTUCAGGACACCA | 6172 | 779-801 |
| AD-1706856.1 | UGUCCUGAAACUGCAACUAGU | 5711 | 782-802 | ACUAGUTGCAGTUUCAGGACACC | 6173 | 780-802 |
| AD-1706857.1 | GUCCUGAAACUGCAACUAGAU | 169 | 783-803 | ATCUAGTUGCAGUUUCAGGACAC | 6174 | 781-803 |
| AD-1706858.1 | UCCUGAAACUGCAACUAGACU | 5712 | 784-804 | AGUCTAGUUGCAGUUUCAGGACA | 6175 | 782-804 |
| AD-1706859.1 | CCUGAAACUGCAACUAGACUU | 5713 | 785-805 | AAGUCUAGUUGCAGUUUCAGGAC | 6176 | 783-805 |
| AD-1706860.1 | CUGAAACUGCAACUAGACUGU | 5714 | 786-806 | ACAGTCTAGUUGCAGUUUCAGGA | 6177 | 784-806 |
| AD-1706861.1 | UGAAACUGCAACUAGACUGCU | 5715 | 787-807 | AGCAGUCUAGUTGCAGUUUCAGG | 6178 | 785-807 |
| AD-1706862.1 | GAAACUGCAACUAGACUGCAU | 5716 | 788-808 | ATGCAGTCUAGTUGCAGUUUCAG | 6179 | 786-808 |
| AD-1706863.1 | AAACUGCAACUAGACUGCAGU | 5717 | 789-809 | ACUGCAGUCUAGUUGCAGUUUCA | 6180 | 787-809 |
| AD-1706864.1 | AACUGCAACUAGACUGCAGAU | 5718 | 790-810 | ATCUGCAGUCUAGUUGCAGUUUC | 6181 | 788-810 |
| AD-1706866.1 | CUGCAACUAGACUGCAGACCU | 5719 | 792-812 | AGGUCUGCAGUCUAGUUGCAGUU | 6182 | 790-812 |
| AD-1706873.1 | GGCAACAGCACAGUUACUGGU | 5720 | 819-839 | ACCAGUAACUGTGCUGUUGCCUU | 6183 | 817-839 |
| AD-1706874.1 | GCAACAGCACAGUUACUGGAU | 5721 | 820-840 | ATCCAGTAACUGUGCUGUUGCCU | 6184 | 818-840 |
| AD-1706875.1 | CAACAGCACAGUUACUGGACU | 5722 | 821-841 | AGUCCAGUAACTGUGCUGUUGCC | 6185 | 819-841 |
| AD-1706876.1 | AACAGCACAGUUACUGGACAU | 5723 | 822-842 | ATGUCCAGUAACUGUGCUGUUGC | 6186 | 820-842 |
| AD-1706877.1 | ACAGCACAGUUACUGGACAAU | 170 | 823-843 | ATUGTCCAGUAACUGUGCUGUUG | 6187 | 821-843 |
| AD-1706878.1 | CAGCACAGUUACUGGACAACU | 5724 | 824-844 | AGUUGUCCAGUAACUGUGCUGUU | 6188 | 822-844 |
| AD-1706879.1 | AGCACAGUUACUGGACAACCU | 5725 | 825-845 | AGGUTGTCCAGTAACUGUGCUGU | 6189 | 823-845 |
| AD-1706908.1 | CUUGGACACAGCAGGACACCU | 5726 | 854-874 | AGGUGUCCUGCTGUGUCCAAGAG | 6190 | 852-874 |
| AD-1706909.1 | UUGGACACAGCAGGACACCAU | 5727 | 855-875 | ATGGTGTCCUGCUGUGUCCAAGA | 6191 | 853-875 |
| AD-1706910.1 | UGGACACAGCAGGACACCAGU | 5728 | 856-876 | ACUGGUGUCCUGCUGUGUCCAAG | 6192 | 854-876 |
| AD-1706911.1 | GGACACAGCAGGACACCAGCU | 5729 | 857-877 | AGCUGGTGUCCTGCUGUGUCCAA | 6193 | 855-877 |
| AD-1706912.1 | GACACAGCAGGACACCAGCAU | 5730 | 858-878 | ATGCTGGUGUCCUGCUGUGUCCA | 6194 | 856-878 |
| AD-1706913.1 | ACACAGCAGGACACCAGCAGU | 5731 | 859-879 | ACUGCUGGUGUCCUGCUGUGUCC | 6195 | 857-879 |
| AD-1706914.1 | CACAGCAGGACACCAGCAGCU | 5732 | 860-880 | AGCUGCTGGUGTCCUGCUGUGUC | 6196 | 858-880 |
| AD-1706918.1 | GCAGGACACCAGCAGCCCUUU | 5733 | 864-884 | AAAGGGCUGCUGGUGUCCUGCUG | 6197 | 862-884 |
| AD-1706921.1 | GGACACCAGCAGCCCUUCCUU | 5734 | 867-887 | AAGGAAGGGCUGCUGGUGUCCUG | 6198 | 865-887 |
| AD-1706922.1 | GACACCAGCAGCCCUUCCUAU | 173 | 868-888 | ATAGGAAGGGCTGCUGGUGUCCU | 6199 | 866-888 |
| AD-1706923.1 | ACACCAGCAGCCCUUCCUAGU | 5735 | 869-889 | ACUAGGAAGGGCUGCUGGUGUCC | 6200 | 867-889 |
| AD-1706925.1 | ACCAGCAGCCCUUCCUAGAGU | 5736 | 871-891 | ACUCTAGGAAGGGCUGCUGGUGU | 6201 | 869-891 |
| AD-1706926.1 | CCAGCAGCCCUUCCUAGAGCU | 5737 | 872-892 | AGCUCUAGGAAGGGCUGCUGGUG | 6202 | 870-892 |
| AD-1706927.1 | CAGCAGCCCUUCCUAGAGCUU | 5738 | 873-893 | AAGCTCTAGGAAGGGCUGCUGGU | 6203 | 871-893 |
| AD-1706929.1 | GCAGCCCUUCCUAGAGCUUAU | 5739 | 875-895 | ATAAGCTCUAGGAAGGGCUGCUG | 6204 | 873-895 |
| AD-1706930.1 | CAGCCCUUCCUAGAGCUUAAU | 5740 | 876-896 | ATUAAGCUCUAGGAAGGGCUGCU | 6205 | 874-896 |
| AD-1706931.1 | AGCCCUUCCUAGAGCUUAAGU | 5741 | 877-897 | ACUUAAGCUCUAGGAAGGGCUGC | 6206 | 875-897 |
| AD-1706932.1 | GCCCUUCCUAGAGCUUAAGAU | 5742 | 878-898 | ATCUTAAGCUCTAGGAAGGGCUG | 6207 | 876-898 |
| AD-1706933.1 | CCCUUCCUAGAGCUUAAGAUU | 5743 | 879-899 | AAUCTUAAGCUCUAGGAAGGGCU | 6208 | 877-899 |
| AD-1706934.1 | CCUUCCUAGAGCUUAAGAUCU | 174 | 880-900 | AGAUCUTAAGCTCUAGGAAGGGC | 6209 | 878-900 |
| AD-1706982.1 | UUACGUAGACUUCCAGGAACU | 5744 | 986-1006 | AGUUCCTGGAAGUCUACGUAAUG | 6210 | 984-1006 |
| AD-1706983.1 | UACGUAGACUUCCAGGAACUU | 178 | 987-1007 | AAGUTCCUGGAAGUCUACGUAAU | 6211 | 985-1007 |
| AD-1706984.1 | ACGUAGACUUCCAGGAACUGU | 5745 | 988-1008 | ACAGTUCCUGGAAGUCUACGUAA | 6212 | 986-1008 |
| AD-1706985.1 | CGUAGACUUCCAGGAACUGGU | 5746 | 989-1009 | ACCAGUTCCUGGAAGUCUACGUA | 6213 | 987-1009 |
| AD-1706987.1 | UAGACUUCCAGGAACUGGGAU | 5747 | 991-1011 | ATCCCAGUUCCTGGAAGUCUACG | 6214 | 989-1011 |
| AD-1706988.1 | AGACUUCCAGGAACUGGGAUU | 5748 | 992-1012 | AAUCCCAGUUCCUGGAAGUCUAC | 6215 | 990-1012 |
| AD-1706989.1 | GACUUCCAGGAACUGGGAUGU | 5749 | 993-1013 | ACAUCCCAGUUCCUGGAAGUCUA | 6216 | 991-1013 |
| AD-1706990.1 | ACUUCCAGGAACUGGGAUGGU | 5750 | 994-1014 | ACCATCCCAGUTCCUGGAAGUCU | 6217 | 992-1014 |
| AD-1706991.1 | CUUCCAGGAACUGGGAUGGCU | 5751 | 995-1015 | AGCCAUCCCAGTUCCUGGAAGUC | 6218 | 993-1015 |
| AD-1707013.1 | GACUGGAUACUGCAGCCCGAU | 5752 | 1017-1037 | ATCGGGCUGCAGUAUCCAGUCCC | 6219 | 1015-1037 |
| AD-1707014.1 | ACUGGAUACUGCAGCCCGAGU | 5753 | 1018-1038 | ACUCGGGCUGCAGUAUCCAGUCC | 6220 | 1016-1038 |
| AD-1707015.1 | CUGGAUACUGCAGCCCGAGGU | 5754 | 1019-1039 | ACCUCGGGCUGCAGUAUCCAGUC | 6221 | 1017-1039 |
| AD-1707016.1 | GUACCAGCUGAAUUACUGCAU | 5755 | 1040-1060 | ATGCAGTAAUUCAGCUGGUACCC | 6222 | 1038-1060 |
| AD-1707017.1 | UACCAGCUGAAUUACUGCAGU | 181 | 1041-1061 | ACUGCAGUAAUTCAGCUGGUACC | 6223 | 1039-1061 |
| AD-1707018.1 | ACCAGCUGAAUUACUGCAGUU | 5756 | 1042-1062 | AACUGCAGUAATUCAGCUGGUAC | 6224 | 1040-1062 |
| AD-1707019.1 | CCAGCUGAAUUACUGCAGUGU | 5757 | 1043-1063 | ACACTGCAGUAAUUCAGCUGGUA | 6225 | 1041-1063 |
| AD-1707020.1 | CAGCUGAAUUACUGCAGUGGU | 5758 | 1044-1064 | ACCACUGCAGUAAUUCAGCUGGU | 6226 | 1042-1064 |
| AD-1707021.1 | AGCUGAAUUACUGCAGUGGGU | 5759 | 1045-1065 | ACCCACTGCAGTAAUUCAGCUGG | 6227 | 1043-1065 |
| AD-1707022.1 | GCUGAAUUACUGCAGUGGGCU | 5760 | 1046-1066 | AGCCCACUGCAGUAAUUCAGCUG | 6228 | 1044-1066 |
| AD-1707023.1 | CUGAAUUACUGCAGUGGGCAU | 5761 | 1047-1067 | ATGCCCACUGCAGUAAUUCAGCU | 6229 | 1045-1067 |
| AD-1707024.1 | UGAAUUACUGCAGUGGGCAGU | 5762 | 1048-1068 | ACUGCCCACUGCAGUAAUUCAGC | 6230 | 1046-1068 |
| AD-1707025.1 | GAAUUACUGCAGUGGGCAGUU | 5763 | 1049-1069 | AACUGCCCACUGCAGUAAUUCAG | 6231 | 1047-1069 |
| AD-1707030.1 | ACUGCAGUGGGCAGUGCCCUU | 182 | 1054-1074 | AAGGGCACUGCCCACUGCAGUAA | 317 | 1052-1074 |
| AD-1707047.1 | CAGGCAUUGCUGCCUCUUUCU | 183 | 1093-1113 | AGAAAGAGGCAGCAAUGCCUGGG | 318 | 1091-1113 |
| AD-1707048.1 | AGGCAUUGCUGCCUCUUUCCU | 5764 | 1094-1114 | AGGAAAGAGGCAGCAAUGCCUGG | 6232 | 1092-1114 |
| AD-1707049.1 | GGCAUUGCUGCCUCUUUCCAU | 5765 | 1095-1115 | ATGGAAAGAGGCAGCAAUGCCUG | 6233 | 1093-1115 |
| AD-1707050.1 | GCAUUGCUGCCUCUUUCCAUU | 5766 | 1096-1116 | AAUGGAAAGAGGCAGCAAUGCCU | 6234 | 1094-1116 |
| AD-1707051.1 | CAUUGCUGCCUCUUUCCAUUU | 5767 | 1097-1117 | AAAUGGAAAGAGGCAGCAAUGCC | 6235 | 1095-1117 |
| AD-1707052.1 | AUUGCUGCCUCUUUCCAUUCU | 5768 | 1098-1118 | AGAATGGAAAGAGGCAGCAAUGC | 6236 | 1096-1118 |
| AD-1707053.1 | UUGCUGCCUCUUUCCAUUCUU | 5769 | 1099-1119 | AAGAAUGGAAAGAGGCAGCAAUG | 6237 | 1097-1119 |
| AD-1707054.1 | UGCUGCCUCUUUCCAUUCUGU | 5770 | 1100-1120 | ACAGAATGGAAAGAGGCAGCAAU | 6238 | 1098-1120 |
| AD-1707055.1 | GCUGCCUCUUUCCAUUCUGCU | 5771 | 1101-1121 | AGCAGAAUGGAAAGAGGCAGCAA | 6239 | 1099-1121 |
| AD-1707056.1 | CUGCCUCUUUCCAUUCUGCCU | 5772 | 1102-1122 | AGGCAGAAUGGAAAGAGGCAGCA | 6240 | 1100-1122 |
| AD-1707057.1 | UGCCUCUUUCCAUUCUGCCGU | 5773 | 1103-1123 | ACGGCAGAAUGGAAAGAGGCAGC | 6241 | 1101-1123 |
| AD-1707058.1 | GCCUCUUUCCAUUCUGCCGUU | 5774 | 1104-1124 | AACGGCAGAAUGGAAAGAGGCAG | 6242 | 1102-1124 |
| AD-1707059.1 | CCUCUUUCCAUUCUGCCGUCU | 5775 | 1105-1125 | AGACGGCAGAATGGAAAGAGGCA | 6243 | 1103-1125 |
| AD-1707060.1 | CUCUUUCCAUUCUGCCGUCUU | 5776 | 1106-1126 | AAGACGGCAGAAUGGAAAGAGGC | 6244 | 1104-1126 |
| AD-1707061.1 | UCUUUCCAUUCUGCCGUCUUU | 5777 | 1107-1127 | AAAGACGGCAGAAUGGAAAGAGG | 6245 | 1105-1127 |
| AD-1707062.1 | CUUUCCAUUCUGCCGUCUUCU | 5778 | 1108-1128 | AGAAGACGGCAGAAUGGAAAGAG | 6246 | 1106-1128 |
| AD-1707063.1 | UUUCCAUUCUGCCGUCUUCAU | 184 | 1109-1129 | ATGAAGACGGCAGAAUGGAAAGA | 6247 | 1107-1129 |
| AD-1707064.1 | UUCCAUUCUGCCGUCUUCAGU | 5779 | 1110-1130 | ACUGAAGACGGCAGAAUGGAAAG | 6248 | 1108-1130 |
| AD-1707065.1 | UCCAUUCUGCCGUCUUCAGCU | 5780 | 1111-1131 | AGCUGAAGACGGCAGAAUGGAAA | 6249 | 1109-1131 |
| AD-1707066.1 | CCAUUCUGCCGUCUUCAGCCU | 5781 | 1112-1132 | AGGCTGAAGACGGCAGAAUGGAA | 6250 | 1110-1132 |
| AD-1707067.1 | CAUUCUGCCGUCUUCAGCCUU | 5782 | 1113-1133 | AAGGCUGAAGACGGCAGAAUGGA | 6251 | 1111-1133 |
| AD-1707070.1 | UCUGCCGUCUUCAGCCUCCUU | 5783 | 1116-1136 | AAGGAGGCUGAAGACGGCAGAAU | 6252 | 1114-1136 |
| AD-1707074.1 | CCGUCUUCAGCCUCCUCAAAU | 5784 | 1120-1140 | ATUUGAGGAGGCUGAAGACGGCA | 6253 | 1118-1140 |
| AD-1707075.1 | CGUCUUCAGCCUCCUCAAAGU | 185 | 1121-1141 | ACUUTGAGGAGGCUGAAGACGGC | 6254 | 1119-1141 |
| AD-1707076.1 | GUCUUCAGCCUCCUCAAAGCU | 5785 | 1122-1142 | AGCUTUGAGGAGGCUGAAGACGG | 6255 | 1120-1142 |
| AD-1707077.1 | UCUUCAGCCUCCUCAAAGCCU | 5786 | 1123-1143 | AGGCTUTGAGGAGGCUGAAGACG | 6256 | 1121-1143 |
| AD-1707078.1 | CUUCAGCCUCCUCAAAGCCAU | 5787 | 1124-1144 | ATGGCUTUGAGGAGGCUGAAGAC | 6257 | 1122-1144 |
| AD-1707080.1 | UCAGCCUCCUCAAAGCCAACU | 5788 | 1126-1146 | AGUUGGCUUUGAGGAGGCUGAAG | 6258 | 1124-1146 |
| AD-1707081.1 | CAGCCUCCUCAAAGCCAACAU | 5789 | 1127-1147 | ATGUTGGCUUUGAGGAGGCUGAA | 6259 | 1125-1147 |
| AD-1707082.1 | AGCCUCCUCAAAGCCAACAAU | 5790 | 1128-1148 | ATUGTUGGCUUTGAGGAGGCUGA | 6260 | 1126-1148 |
| AD-1707083.1 | GCCUCCUCAAAGCCAACAAUU | 5791 | 1129-1149 | AAUUGUTGGCUTUGAGGAGGCUG | 6261 | 1127-1149 |
| AD-1707084.1 | CCUCCUCAAAGCCAACAAUCU | 5792 | 1130-1150 | AGAUTGTUGGCTUUGAGGAGGCU | 6262 | 1128-1150 |
| AD-1707085.1 | CUCCUCAAAGCCAACAAUCCU | 5793 | 1131-1151 | AGGATUGUUGGCUUUGAGGAGGC | 6263 | 1129-1151 |
| AD-1707086.1 | UCCUCAAAGCCAACAAUCCUU | 5794 | 1132-1152 | AAGGAUTGUUGGCUUUGAGGAGG | 6264 | 1130-1152 |
| AD-1707087.1 | CCUCAAAGCCAACAAUCCUUU | 186 | 1133-1153 | AAAGGATUGUUGGCUUUGAGGAG | 6265 | 1131-1153 |
| AD-1707088.1 | CUCAAAGCCAACAAUCCUUGU | 5795 | 1134-1154 | ACAAGGAUUGUTGGCUUUGAGGA | 6266 | 1132-1154 |
| AD-1707092.1 | AAGCCAACAAUCCUUGGCCUU | 5796 | 1138-1158 | AAGGCCAAGGATUGUUGGCUUUG | 6267 | 1136-1158 |
| AD-1707093.1 | AGCCAACAAUCCUUGGCCUGU | 5797 | 1139-1159 | ACAGGCCAAGGAUUGUUGGCUUU | 6268 | 1137-1159 |
| AD-1707094.1 | GCCAACAAUCCUUGGCCUGCU | 5798 | 1140-1160 | AGCAGGCCAAGGAUUGUUGGCUU | 6269 | 1138-1160 |
| AD-1707095.1 | CCAACAAUCCUUGGCCUGCCU | 5799 | 1141-1161 | AGGCAGGCCAAGGAUUGUUGGCU | 6270 | 1139-1161 |
| AD-1707097.1 | AACAAUCCUUGGCCUGCCAGU | 5800 | 1143-1163 | ACUGGCAGGCCAAGGAUUGUUGG | 6271 | 1141-1163 |
| AD-1707098.1 | ACAAUCCUUGGCCUGCCAGUU | 5801 | 1144-1164 | AACUGGCAGGCCAAGGAUUGUUG | 6272 | 1142-1164 |
| AD-1707105.1 | UUGGCCUGCCAGUACCUCCUU | 5802 | 1151-1171 | AAGGAGGUACUGGCAGGCCAAGG | 6273 | 1149-1171 |
| AD-1707125.1 | GUUGUGUCCCUACUGCCCGAU | 5803 | 1171-1191 | ATCGGGCAGUAGGGACACAACAG | 6274 | 1169-1191 |
| AD-1707126.1 | UUGUGUCCCUACUGCCCGAAU | 5804 | 1172-1192 | ATUCGGGCAGUAGGGACACAACA | 6275 | 1170-1192 |
| AD-1707127.1 | UGUGUCCCUACUGCCCGAAGU | 5805 | 1173-1193 | ACUUCGGGCAGTAGGGACACAAC | 6276 | 1171-1193 |
| AD-1707128.1 | GUGUCCCUACUGCCCGAAGGU | 189 | 1174-1194 | ACCUTCGGGCAGUAGGGACACAA | 6277 | 1172-1194 |
| AD-1707129.1 | UGUCCCUACUGCCCGAAGGCU | 5806 | 1175-1195 | AGCCTUCGGGCAGUAGGGACACA | 6278 | 1173-1195 |
| AD-1707132.1 | UCUCUCUCCUCUACCUGGAUU | 5807 | 1198-1218 | AAUCCAGGUAGAGGAGAGAGAGG | 6279 | 1196-1218 |
| AD-1707133.1 | CUCUCUCCUCUACCUGGAUCU | 5808 | 1199-1219 | AGAUCCAGGUAGAGGAGAGAGAG | 6280 | 1197-1219 |
| AD-1707134.1 | UCUCUCCUCUACCUGGAUCAU | 5809 | 1200-1220 | ATGATCCAGGUAGAGGAGAGAGA | 6281 | 1198-1220 |
| AD-1707135.1 | CUCUCCUCUACCUGGAUCAUU | 5810 | 1201-1221 | AAUGAUCCAGGTAGAGGAGAGAG | 6282 | 1199-1221 |
| AD-1707136.1 | UCUCCUCUACCUGGAUCAUAU | 5811 | 1202-1222 | ATAUGATCCAGGUAGAGGAGAGA | 6283 | 1200-1222 |
| AD-1707137.1 | CUCCUCUACCUGGAUCAUAAU | 5812 | 1203-1223 | ATUATGAUCCAGGUAGAGGAGAG | 6284 | 1201-1223 |
| AD-1707138.1 | UCCUCUACCUGGAUCAUAAUU | 5813 | 1204-1224 | AAUUAUGAUCCAGGUAGAGGAGA | 6285 | 1202-1224 |
| AD-1707139.1 | CCUCUACCUGGAUCAUAAUGU | 5814 | 1205-1225 | ACAUTATGAUCCAGGUAGAGGAG | 6286 | 1203-1225 |
| AD-1707140.1 | CUCUACCUGGAUCAUAAUGGU | 5815 | 1206-1226 | ACCATUAUGAUCCAGGUAGAGGA | 6287 | 1204-1226 |
| AD-1707141.1 | UCUACCUGGAUCAUAAUGGCU | 5816 | 1207-1227 | AGCCAUTAUGATCCAGGUAGAGG | 6288 | 1205-1227 |
| AD-1707142.1 | CUACCUGGAUCAUAAUGGCAU | 5817 | 1208-1228 | ATGCCATUAUGAUCCAGGUAGAG | 6289 | 1206-1228 |
| AD-1707143.1 | UACCUGGAUCAUAAUGGCAAU | 191 | 1209-1229 | ATUGCCAUUAUGAUCCAGGUAGA | 6290 | 1207-1229 |
| AD-1707144.1 | ACCUGGAUCAUAAUGGCAAUU | 5818 | 1210-1230 | AAUUGCCAUUATGAUCCAGGUAG | 6291 | 1208-1230 |
| AD-1707145.1 | CCUGGAUCAUAAUGGCAAUGU | 5819 | 1211-1231 | ACAUTGCCAUUAUGAUCCAGGUA | 6292 | 1209-1231 |
| AD-1707146.1 | CUGGAUCAUAAUGGCAAUGUU | 5820 | 1212-1232 | AACATUGCCAUTAUGAUCCAGGU | 6293 | 1210-1232 |
| AD-1707147.1 | UGGAUCAUAAUGGCAAUGUGU | 5821 | 1213-1233 | ACACAUTGCCATUAUGAUCCAGG | 6294 | 1211-1233 |
| AD-1707148.1 | GGAUCAUAAUGGCAAUGUGGU | 5822 | 1214-1234 | ACCACATUGCCAUUAUGAUCCAG | 6295 | 1212-1234 |
| AD-1707149.1 | GAUCAUAAUGGCAAUGUGGUU | 5823 | 1215-1235 | AACCACAUUGCCAUUAUGAUCCA | 6296 | 1213-1235 |
| AD-1707150.1 | AUCAUAAUGGCAAUGUGGUCU | 5824 | 1216-1236 | AGACCACAUUGCCAUUAUGAUCC | 6297 | 1214-1236 |
| AD-1707151.1 | UCAUAAUGGCAAUGUGGUCAU | 5825 | 1217-1237 | ATGACCACAUUGCCAUUAUGAUC | 6298 | 1215-1237 |
| AD-1707152.1 | CAUAAUGGCAAUGUGGUCAAU | 5826 | 1218-1238 | ATUGACCACAUTGCCAUUAUGAU | 6299 | 1216-1238 |
| AD-1707153.1 | AUAAUGGCAAUGUGGUCAAGU | 5827 | 1219-1239 | ACUUGACCACATUGCCAUUAUGA | 6300 | 1217-1239 |
| AD-1707154.1 | UAAUGGCAAUGUGGUCAAGAU | 5828 | 1220-1240 | ATCUTGACCACAUUGCCAUUAUG | 6301 | 1218-1240 |
| AD-1707155.1 | AAUGGCAAUGUGGUCAAGACU | 192 | 1221-1241 | AGUCTUGACCACAUUGCCAUUAU | 6302 | 1219-1241 |
| AD-1707156.1 | AUGGCAAUGUGGUCAAGACGU | 5829 | 1222-1242 | ACGUCUTGACCACAUUGCCAUUA | 6303 | 1220-1242 |
| AD-1707157.1 | UGGCAAUGUGGUCAAGACGGU | 5830 | 1223-1243 | ACCGTCTUGACCACAUUGCCAUU | 6304 | 1221-1243 |
| AD-1707158.1 | GGCAAUGUGGUCAAGACGGAU | 5831 | 1224-1244 | ATCCGUCUUGACCACAUUGCCAU | 6305 | 1222-1244 |
| AD-1707159.1 | GCAAUGUGGUCAAGACGGAUU | 5832 | 1225-1245 | AAUCCGTCUUGACCACAUUGCCA | 6306 | 1223-1245 |
| AD-1707160.1 | CAAUGUGGUCAAGACGGAUGU | 5833 | 1226-1246 | ACAUCCGUCUUGACCACAUUGCC | 6307 | 1224-1246 |
| AD-1707161.1 | AAUGUGGUCAAGACGGAUGUU | 5834 | 1227-1247 | AACATCCGUCUTGACCACAUUGC | 6308 | 1225-1247 |
| AD-1707162.1 | AUGUGGUCAAGACGGAUGUGU | 5835 | 1228-1248 | ACACAUCCGUCTUGACCACAUUG | 6309 | 1226-1248 |
| AD-1707163.1 | UGUGGUCAAGACGGAUGUGCU | 5836 | 1229-1249 | AGCACATCCGUCUUGACCACAUU | 6310 | 1227-1249 |
| AD-1707164.1 | GUGGUCAAGACGGAUGUGCCU | 5837 | 1230-1250 | AGGCACAUCCGTCUUGACCACAU | 6311 | 1228-1250 |
| AD-1707165.1 | UGGUCAAGACGGAUGUGCCAU | 5838 | 1231-1251 | ATGGCACAUCCGUCUUGACCACA | 6312 | 1229-1251 |
| AD-1707166.1 | GGUCAAGACGGAUGUGCCAGU | 5839 | 1232-1252 | ACUGGCACAUCCGUCUUGACCAC | 6313 | 1230-1252 |
| AD-1707167.1 | GUCAAGACGGAUGUGCCAGAU | 5840 | 1233-1253 | ATCUGGCACAUCCGUCUUGACCA | 6314 | 1231-1253 |
| AD-1707168.1 | UCAAGACGGAUGUGCCAGAUU | 5841 | 1234-1254 | AAUCTGGCACATCCGUCUUGACC | 6315 | 1232-1254 |
| AD-1707169.1 | CAAGACGGAUGUGCCAGAUAU | 5842 | 1235-1255 | ATAUCUGGCACAUCCGUCUUGAC | 6316 | 1233-1255 |
| AD-1707170.1 | AAGACGGAUGUGCCAGAUAUU | 193 | 1236-1256 | AAUATCTGGCACAUCCGUCUUGA | 6317 | 1234-1256 |
| AD-1707171.1 | AGACGGAUGUGCCAGAUAUGU | 5843 | 1237-1257 | ACAUAUCUGGCACAUCCGUCUUG | 6318 | 1235-1257 |
| AD-1707172.1 | GACGGAUGUGCCAGAUAUGGU | 5844 | 1238-1258 | ACCATATCUGGCACAUCCGUCUU | 6319 | 1236-1258 |
| AD-1707173.1 | ACGGAUGUGCCAGAUAUGGUU | 5845 | 1239-1259 | AACCAUAUCUGGCACAUCCGUCU | 6320 | 1237-1259 |
| AD-1707174.1 | CGGAUGUGCCAGAUAUGGUGU | 5846 | 1240-1260 | ACACCATAUCUGGCACAUCCGUC | 6321 | 1238-1260 |
| AD-1707176.1 | GAUGUGCCAGAUAUGGUGGUU | 5847 | 1242-1262 | AACCACCAUAUCUGGCACAUCCG | 6322 | 1240-1262 |
| AD-1707177.1 | AUGUGCCAGAUAUGGUGGUGU | 5848 | 1243-1263 | ACACCACCAUATCUGGCACAUCC | 6323 | 1241-1263 |
| AD-1707178.1 | UGUGCCAGAUAUGGUGGUGGU | 5849 | 1244-1264 | ACCACCACCAUAUCUGGCACAUC | 6324 | 1242-1264 |
| AD-1707179.1 | GUGCCAGAUAUGGUGGUGGAU | 5850 | 1245-1265 | ATCCACCACCATAUCUGGCACAU | 6325 | 1243-1265 |
| AD-1707180.1 | UGCCAGAUAUGGUGGUGGAGU | 5851 | 1246-1266 | ACUCCACCACCAUAUCUGGCACA | 6326 | 1244-1266 |
| AD-1707182.1 | CCAGAUAUGGUGGUGGAGGCU | 5852 | 1248-1268 | AGCCTCCACCACCAUAUCUGGCA | 6327 | 1246-1268 |
| AD-1707189.1 | UGGUGGUGGAGGCCUGUGGCU | 5853 | 1255-1275 | AGCCACAGGCCTCCACCACCAUA | 6328 | 1253-1275 |
| AD-1707190.1 | GGUGGUGGAGGCCUGUGGCUU | 5854 | 1256-1276 | AAGCCACAGGCCUCCACCACCAU | 6329 | 1254-1276 |
| AD-1707197.1 | GAGGCCUGUGGCUGCAGCUAU | 195 | 1263-1283 | ATAGCUGCAGCCACAGGCCUCCA | 6330 | 1261-1283 |
| AD-1707200.1 | GCCUGUGGCUGCAGCUAGCAU | 5855 | 1266-1286 | ATGCTAGCUGCAGCCACAGGCCU | 6331 | 1264-1286 |
| AD-1707201.1 | CCUGUGGCUGCAGCUAGCAAU | 5856 | 1267-1287 | ATUGCUAGCUGCAGCCACAGGCC | 6332 | 1265-1287 |
| AD-1707202.1 | CUGUGGCUGCAGCUAGCAAGU | 5857 | 1268-1288 | ACUUGCTAGCUGCAGCCACAGGC | 6333 | 1266-1288 |
| AD-1707203.1 | UGUGGCUGCAGCUAGCAAGAU | 5858 | 1269-1289 | ATCUTGCUAGCTGCAGCCACAGG | 6334 | 1267-1289 |
| AD-1707204.1 | GUGGCUGCAGCUAGCAAGAGU | 5859 | 1270-1290 | ACUCTUGCUAGCUGCAGCCACAG | 6335 | 1268-1290 |
| AD-1707205.1 | UGGCUGCAGCUAGCAAGAGGU | 5860 | 1271-1291 | ACCUCUTGCUAGCUGCAGCCACA | 6336 | 1269-1291 |
| AD-1707206.1 | GGCUGCAGCUAGCAAGAGGAU | 5861 | 1272-1292 | ATCCTCTUGCUAGCUGCAGCCAC | 6337 | 1270-1292 |
| AD-1707207.1 | GCUGCAGCUAGCAAGAGGACU | 5862 | 1273-1293 | AGUCCUCUUGCTAGCUGCAGCCA | 6338 | 1271-1293 |
| AD-1707208.1 | CUGCAGCUAGCAAGAGGACCU | 5863 | 1274-1294 | AGGUCCTCUUGCUAGCUGCAGCC | 6339 | 1272-1294 |
| AD-1707209.1 | UGCAGCUAGCAAGAGGACCUU | 196 | 1275-1295 | AAGGTCCUCUUGCUAGCUGCAGC | 6340 | 1273-1295 |
| AD-1707210.1 | GCAGCUAGCAAGAGGACCUGU | 5864 | 1276-1296 | ACAGGUCCUCUTGCUAGCUGCAG | 6341 | 1274-1296 |
| AD-1707211.1 | CAGCUAGCAAGAGGACCUGGU | 5865 | 1277-1297 | ACCAGGTCCUCTUGCUAGCUGCA | 6342 | 1275-1297 |
| AD-1707212.1 | GCUUUGGAGUGAAGAGACCAU | 5866 | 1298-1318 | ATGGTCTCUUCACUCCAAAGCCC | 6343 | 1296-1318 |
| AD-1707213.1 | CUUUGGAGUGAAGAGACCAAU | 197 | 1299-1319 | ATUGGUCUCUUCACUCCAAAGCC | 6344 | 1297-1319 |
| AD-1707214.1 | UUUGGAGUGAAGAGACCAAGU | 5867 | 1300-1320 | ACUUGGTCUCUTCACUCCAAAGC | 6345 | 1298-1320 |
| AD-1707216.1 | UGGAGUGAAGAGACCAAGAUU | 5868 | 1302-1322 | AAUCTUGGUCUCUUCACUCCAAA | 6346 | 1300-1322 |
| AD-1707217.1 | GGAGUGAAGAGACCAAGAUGU | 5869 | 1303-1323 | ACAUCUTGGUCTCUUCACUCCAA | 6347 | 1301-1323 |
| AD-1707218.1 | GAGUGAAGAGACCAAGAUGAU | 5870 | 1304-1324 | ATCATCTUGGUCUCUUCACUCCA | 6348 | 1302-1324 |
| AD-1707221.1 | UGAAGAGACCAAGAUGAAGUU | 5871 | 1307-1327 | AACUTCAUCUUGGUCUCUUCACU | 6349 | 1305-1327 |
| AD-1707223.1 | AAGAGACCAAGAUGAAGUUUU | 5872 | 1309-1329 | AAAACUTCAUCTUGGUCUCUUCA | 6350 | 1307-1329 |
| AD-1707224.1 | AGAGACCAAGAUGAAGUUUCU | 198 | 1310-1330 | AGAAACTUCAUCUUGGUCUCUUC | 6351 | 1308-1330 |
| AD-1707225.1 | GAGACCAAGAUGAAGUUUCCU | 5873 | 1311-1331 | AGGAAACUUCATCUUGGUCUCUU | 6352 | 1309-1331 |
| AD-1707226.1 | AGACCAAGAUGAAGUUUCCCU | 5874 | 1312-1332 | AGGGAAACUUCAUCUUGGUCUCU | 6353 | 1310-1332 |
| AD-1707227.1 | GACCAAGAUGAAGUUUCCCAU | 5875 | 1313-1333 | ATGGGAAACUUCAUCUUGGUCUC | 6354 | 1311-1333 |
| AD-1707228.1 | ACCAAGAUGAAGUUUCCCAGU | 5876 | 1314-1334 | ACUGGGAAACUTCAUCUUGGUCU | 6355 | 1312-1334 |
| AD-1707229.1 | CCAAGAUGAAGUUUCCCAGGU | 5877 | 1315-1335 | ACCUGGGAAACTUCAUCUUGGUC | 6356 | 1313-1335 |
| AD-1707230.1 | CAAGAUGAAGUUUCCCAGGCU | 5878 | 1316-1336 | AGCCTGGGAAACUUCAUCUUGGU | 6357 | 1314-1336 |
| AD-1707231.1 | AAGAUGAAGUUUCCCAGGCAU | 5879 | 1317-1337 | ATGCCUGGGAAACUUCAUCUUGG | 6358 | 1315-1337 |
| AD-1707232.1 | AGAUGAAGUUUCCCAGGCACU | 5880 | 1318-1338 | AGUGCCTGGGAAACUUCAUCUUG | 6359 | 1316-1338 |
| AD-1707233.1 | GAUGAAGUUUCCCAGGCACAU | 5881 | 1319-1339 | ATGUGCCUGGGAAACUUCAUCUU | 6360 | 1317-1339 |
| AD-1707234.1 | AUGAAGUUUCCCAGGCACAGU | 5882 | 1320-1340 | ACUGTGCCUGGGAAACUUCAUCU | 6361 | 1318-1340 |
| AD-1707236.1 | GAAGUUUCCCAGGCACAGGGU | 5883 | 1322-1342 | ACCCTGTGCCUGGGAAACUUCAU | 6362 | 1320-1342 |
| AD-1707237.1 | AAGUUUCCCAGGCACAGGGCU | 5884 | 1323-1343 | AGCCCUGUGCCTGGGAAACUUCA | 6363 | 1321-1343 |
| AD-1707239.1 | GUUUCCCAGGCACAGGGCAUU | 5885 | 1325-1345 | AAUGCCCUGUGCCUGGGAAACUU | 6364 | 1323-1345 |
| AD-1707240.1 | UUUCCCAGGCACAGGGCAUCU | 5886 | 1326-1346 | AGAUGCCCUGUGCCUGGGAAACU | 6365 | 1324-1346 |
| AD-1707241.1 | UUCCCAGGCACAGGGCAUCUU | 5887 | 1327-1347 | AAGATGCCCUGTGCCUGGGAAAC | 6366 | 1325-1347 |
| AD-1707242.1 | UCCCAGGCACAGGGCAUCUGU | 5888 | 1328-1348 | ACAGAUGCCCUGUGCCUGGGAAA | 6367 | 1326-1348 |
| AD-1707275.1 | CAACCCAACAACCACCUGGCU | 5889 | 1381-1401 | AGCCAGGUGGUTGUUGGGUUGGG | 6368 | 1379-1401 |
| AD-1707276.1 | AACCCAACAACCACCUGGCAU | 5890 | 1382-1402 | ATGCCAGGUGGTUGUUGGGUUGG | 6369 | 1380-1402 |
| AD-1707277.1 | ACCCAACAACCACCUGGCAAU | 201 | 1383-1403 | ATUGCCAGGUGGUUGUUGGGUUG | 6370 | 1381-1403 |
| AD-1707278.1 | CCCAACAACCACCUGGCAAUU | 5891 | 1384-1404 | AAUUGCCAGGUGGUUGUUGGGUU | 6371 | 1382-1404 |
| AD-1707279.1 | CCAACAACCACCUGGCAAUAU | 5892 | 1385-1405 | ATAUTGCCAGGTGGUUGUUGGGU | 6372 | 1383-1405 |
| AD-1707280.1 | CAACAACCACCUGGCAAUAUU | 5893 | 1386-1406 | AAUATUGCCAGGUGGUUGUUGGG | 6373 | 1384-1406 |
| AD-1707281.1 | AACAACCACCUGGCAAUAUGU | 5894 | 1387-1407 | ACAUAUTGCCAGGUGGUUGUUGG | 6374 | 1385-1407 |
| AD-1707282.1 | ACAACCACCUGGCAAUAUGAU | 5895 | 1388-1408 | ATCATATUGCCAGGUGGUUGUUG | 6375 | 1386-1408 |
| AD-1707283.1 | CAACCACCUGGCAAUAUGACU | 5896 | 1389-1409 | AGUCAUAUUGCCAGGUGGUUGUU | 6376 | 1387-1409 |
| AD-1707284.1 | AACCACCUGGCAAUAUGACUU | 5897 | 1390-1410 | AAGUCATAUUGCCAGGUGGUUGU | 6377 | 1388-1410 |
| AD-1707285.1 | ACCACCUGGCAAUAUGACUCU | 5898 | 1391-1411 | AGAGTCAUAUUGCCAGGUGGUUG | 6378 | 1389-1411 |
| AD-1707286.1 | CCACCUGGCAAUAUGACUCAU | 5899 | 1392-1412 | ATGAGUCAUAUTGCCAGGUGGUU | 6379 | 1390-1412 |
| AD-1707287.1 | CACCUGGCAAUAUGACUCACU | 5900 | 1393-1413 | AGUGAGTCAUATUGCCAGGUGGU | 6380 | 1391-1413 |
| AD-1707288.1 | ACCUGGCAAUAUGACUCACUU | 5901 | 1394-1414 | AAGUGAGUCAUAUUGCCAGGUGG | 6381 | 1392-1414 |
| AD-1707289.1 | CCUGGCAAUAUGACUCACUUU | 5902 | 1395-1415 | AAAGTGAGUCATAUUGCCAGGUG | 6382 | 1393-1415 |
| AD-1707290.1 | CUGGCAAUAUGACUCACUUGU | 5903 | 1396-1416 | ACAAGUGAGUCAUAUUGCCAGGU | 6383 | 1394-1416 |
| AD-1707291.1 | UGGCAAUAUGACUCACUUGAU | 5904 | 1397-1417 | ATCAAGTGAGUCAUAUUGCCAGG | 6384 | 1395-1417 |
| AD-1707292.1 | GGCAAUAUGACUCACUUGACU | 202 | 1398-1418 | AGUCAAGUGAGTCAUAUUGCCAG | 6385 | 1396-1418 |
| AD-1707293.1 | GCAAUAUGACUCACUUGACCU | 5905 | 1399-1419 | AGGUCAAGUGAGUCAUAUUGCCA | 6386 | 1397-1419 |
| AD-1707299.1 | GGACCCAAAUGGGCACUUUCU | 5906 | 1425-1445 | AGAAAGTGCCCAUUUGGGUCCCA | 6387 | 1423-1445 |
| AD-1707301.1 | ACCCAAAUGGGCACUUUCUUU | 5907 | 1427-1447 | AAAGAAAGUGCCCAUUUGGGUCC | 6388 | 1425-1447 |
| AD-1707302.1 | CCCAAAUGGGCACUUUCUUGU | 5908 | 1428-1448 | ACAAGAAAGUGCCCAUUUGGGUC | 6389 | 1426-1448 |
| AD-1707303.1 | CCAAAUGGGCACUUUCUUGUU | 5909 | 1429-1449 | AACAAGAAAGUGCCCAUUUGGGU | 6390 | 1427-1449 |
| AD-1707304.1 | CAAAUGGGCACUUUCUUGUCU | 5910 | 1430-1450 | AGACAAGAAAGTGCCCAUUUGGG | 6391 | 1428-1450 |
| AD-1707305.1 | AAAUGGGCACUUUCUUGUCUU | 5911 | 1431-1451 | AAGACAAGAAAGUGCCCAUUUGG | 6392 | 1429-1451 |
| AD-1707306.1 | AAUGGGCACUUUCUUGUCUGU | 71 | 1432-1452 | ACAGACAAGAAAGUGCCCAUUUG | 63 | 1430-1452 |
| AD-1707307.1 | AUGGGCACUUUCUUGUCUGAU | 5912 | 1433-1453 | ATCAGACAAGAAAGUGCCCAUUU | 6393 | 1431-1453 |
| AD-1707308.1 | UGGGCACUUUCUUGUCUGAGU | 5913 | 1434-1454 | ACUCAGACAAGAAAGUGCCCAUU | 6394 | 1432-1454 |
| AD-1707309.1 | GGGCACUUUCUUGUCUGAGAU | 5914 | 1435-1455 | ATCUCAGACAAGAAAGUGCCCAU | 6395 | 1433-1455 |
| AD-1707310.1 | GGCACUUUCUUGUCUGAGACU | 5915 | 1436-1456 | AGUCTCAGACAAGAAAGUGCCCA | 6396 | 1434-1456 |
| AD-1707311.1 | GCACUUUCUUGUCUGAGACUU | 5916 | 1437-1457 | AAGUCUCAGACAAGAAAGUGCCC | 6397 | 1435-1457 |
| AD-1707312.1 | CACUUUCUUGUCUGAGACUCU | 5917 | 1438-1458 | AGAGTCTCAGACAAGAAAGUGCC | 6398 | 1436-1458 |
| AD-1707313.1 | ACUUUCUUGUCUGAGACUCUU | 204 | 1439-1459 | AAGAGUCUCAGACAAGAAAGUGC | 339 | 1437-1459 |
| AD-1707314.1 | CUUUCUUGUCUGAGACUCUGU | 5918 | 1440-1460 | ACAGAGTCUCAGACAAGAAAGUG | 6399 | 1438-1460 |
| AD-1707315.1 | UUUCUUGUCUGAGACUCUGGU | 5919 | 1441-1461 | ACCAGAGUCUCAGACAAGAAAGU | 6400 | 1439-1461 |
| AD-1707316.1 | UUCUUGUCUGAGACUCUGGCU | 5920 | 1442-1462 | AGCCAGAGUCUCAGACAAGAAAG | 6401 | 1440-1462 |
| AD-1707317.1 | UCUUGUCUGAGACUCUGGCUU | 5921 | 1443-1463 | AAGCCAGAGUCTCAGACAAGAAA | 6402 | 1441-1463 |
| AD-1707318.1 | CUUGUCUGAGACUCUGGCUUU | 5922 | 1444-1464 | AAAGCCAGAGUCUCAGACAAGAA | 6403 | 1442-1464 |
| AD-1707337.1 | UAUUCCAGGUUGGCUGAUGUU | 5923 | 1463-1483 | AACATCAGCCAACCUGGAAUAAG | 6404 | 1461-1483 |
| AD-1707338.1 | AUUCCAGGUUGGCUGAUGUGU | 5924 | 1464-1484 | ACACAUCAGCCAACCUGGAAUAA | 6405 | 1462-1484 |
| AD-1707339.1 | UUCCAGGUUGGCUGAUGUGUU | 5925 | 1465-1485 | AACACATCAGCCAACCUGGAAUA | 6406 | 1463-1485 |
| AD-1707340.1 | UCCAGGUUGGCUGAUGUGUUU | 206 | 1466-1486 | AAACACAUCAGCCAACCUGGAAU | 341 | 1464-1486 |
| AD-1707341.1 | CCAGGUUGGCUGAUGUGUUGU | 5926 | 1467-1487 | ACAACACAUCAGCCAACCUGGAA | 6407 | 1465-1487 |
| AD-1707342.1 | CAGGUUGGCUGAUGUGUUGGU | 5927 | 1468-1488 | ACCAACACAUCAGCCAACCUGGA | 6408 | 1466-1488 |
| AD-1707388.1 | GAUUUCCUGCCCUAAGUCCUU | 210 | 1534-1554 | AAGGACTUAGGGCAGGAAAUCAU | 6409 | 1532-1554 |
| AD-1707390.1 | UUUCCUGCCCUAAGUCCUGUU | 5928 | 1536-1556 | AACAGGACUUAGGGCAGGAAAUC | 6410 | 1534-1556 |
| AD-1707411.1 | AGAAGAUGUCAGGGACUAGGU | 5929 | 1557-1577 | ACCUAGTCCCUGACAUCUUCUCA | 6411 | 1555-1577 |
| AD-1707412.1 | GAAGAUGUCAGGGACUAGGGU | 5930 | 1558-1578 | ACCCTAGUCCCTGACAUCUUCUC | 6412 | 1556-1578 |
| AD-1707415.1 | AAGAUGUCAGGGACUAGGGAU | 5931 | 1559-1579 | ATCCCUAGUCCCUGACAUCUUCU | 6413 | 1557-1579 |
| AD-1707416.1 | AGAUGUCAGGGACUAGGGAGU | 5932 | 1560-1580 | ACUCCCTAGUCCCUGACAUCUUC | 6414 | 1558-1580 |
| AD-1707417.1 | GAUGUCAGGGACUAGGGAGGU | 5933 | 1561-1581 | ACCUCCCUAGUCCCUGACAUCUU | 6415 | 1559-1581 |
| AD-1707418.1 | AUGUCAGGGACUAGGGAGGGU | 5934 | 1562-1582 | ACCCTCCCUAGTCCCUGACAUCU | 6416 | 1560-1582 |
| AD-1707466.1 | GAGGAGGAAGCAGAUAGAUGU | 5935 | 1646-1666 | ACAUCUAUCUGCUUCCUCCUCCC | 6417 | 1644-1666 |
| AD-1707467.1 | AGGAGGAAGCAGAUAGAUGGU | 5936 | 1647-1667 | ACCATCTAUCUGCUUCCUCCUCC | 6418 | 1645-1667 |
| AD-1707468.1 | GGAGGAAGCAGAUAGAUGGUU | 215 | 1648-1668 | AACCAUCUAUCTGCUUCCUCCUC | 6419 | 1646-1668 |
| AD-1707469.1 | GAGGAAGCAGAUAGAUGGUCU | 5937 | 1649-1669 | AGACCATCUAUCUGCUUCCUCCU | 6420 | 1647-1669 |
| AD-1707470.1 | AGGAAGCAGAUAGAUGGUCCU | 5938 | 1650-1670 | AGGACCAUCUATCUGCUUCCUCC | 6421 | 1648-1670 |
| AD-1707471.1 | GGAAGCAGAUAGAUGGUCCAU | 5939 | 1651-1671 | ATGGACCAUCUAUCUGCUUCCUC | 6422 | 1649-1671 |
| AD-1707472.1 | GAAGCAGAUAGAUGGUCCAGU | 5940 | 1652-1672 | ACUGGACCAUCTAUCUGCUUCCU | 6423 | 1650-1672 |
| AD-1707473.1 | AAGCAGAUAGAUGGUCCAGCU | 5941 | 1653-1673 | AGCUGGACCAUCUAUCUGCUUCC | 6424 | 1651-1673 |
| AD-1707474.1 | AGCAGAUAGAUGGUCCAGCAU | 5942 | 1654-1674 | ATGCTGGACCATCUAUCUGCUUC | 6425 | 1652-1674 |
| AD-1707475.1 | GCAGAUAGAUGGUCCAGCAGU | 5943 | 1655-1675 | ACUGCUGGACCAUCUAUCUGCUU | 6426 | 1653-1675 |
| AD-1707476.1 | CAGAUAGAUGGUCCAGCAGGU | 5944 | 1656-1676 | ACCUGCTGGACCAUCUAUCUGCU | 6427 | 1654-1676 |
| AD-1707477.1 | AGAUAGAUGGUCCAGCAGGCU | 5945 | 1657-1677 | AGCCTGCUGGACCAUCUAUCUGC | 6428 | 1655-1677 |
| AD-1707478.1 | GAUAGAUGGUCCAGCAGGCUU | 5946 | 1658-1678 | AAGCCUGCUGGACCAUCUAUCUG | 6429 | 1656-1678 |
| AD-1707479.1 | AUAGAUGGUCCAGCAGGCUUU | 5947 | 1659-1679 | AAAGCCTGCUGGACCAUCUAUCU | 6430 | 1657-1679 |
| AD-1707480.1 | UAGAUGGUCCAGCAGGCUUGU | 5948 | 1660-1680 | ACAAGCCUGCUGGACCAUCUAUC | 6431 | 1658-1680 |
| AD-1707481.1 | AGAUGGUCCAGCAGGCUUGAU | 5949 | 1661-1681 | ATCAAGCCUGCTGGACCAUCUAU | 6432 | 1659-1681 |
| AD-1707482.1 | GAUGGUCCAGCAGGCUUGAAU | 5950 | 1662-1682 | ATUCAAGCCUGCUGGACCAUCUA | 6433 | 1660-1682 |
| AD-1707483.1 | AUGGUCCAGCAGGCUUGAAGU | 5951 | 1663-1683 | ACUUCAAGCCUGCUGGACCAUCU | 6434 | 1661-1683 |
| AD-1707484.1 | UGGUCCAGCAGGCUUGAAGCU | 5952 | 1664-1684 | AGCUTCAAGCCTGCUGGACCAUC | 6435 | 1662-1684 |
| AD-1707485.1 | GGUCCAGCAGGCUUGAAGCAU | 5953 | 1665-1685 | ATGCTUCAAGCCUGCUGGACCAU | 6436 | 1663-1685 |
| AD-1707486.1 | GUCCAGCAGGCUUGAAGCAGU | 5954 | 1666-1686 | ACUGCUTCAAGCCUGCUGGACCA | 6437 | 1664-1686 |
| AD-1707553.1 | AAGGUCAAGAGGGAGAUGGGU | 5955 | 1733-1753 | ACCCAUCUCCCTCUUGACCUUCC | 6438 | 1731-1753 |
| AD-1707554.1 | AGGUCAAGAGGGAGAUGGGCU | 5956 | 1734-1754 | AGCCCATCUCCCUCUUGACCUUC | 6439 | 1732-1754 |
| AD-1707555.1 | GGUCAAGAGGGAGAUGGGCAU | 5957 | 1735-1755 | ATGCCCAUCUCCCUCUUGACCUU | 6440 | 1733-1755 |
| AD-1707556.1 | GUCAAGAGGGAGAUGGGCAAU | 221 | 1736-1756 | ATUGCCCAUCUCCCUCUUGACCU | 6441 | 1734-1756 |
| AD-1707557.1 | UCAAGAGGGAGAUGGGCAAGU | 5958 | 1737-1757 | ACUUGCCCAUCTCCCUCUUGACC | 6442 | 1735-1757 |
| AD-1707577.1 | GCGCUGAGGGAGGAUGCUUAU | 5959 | 1757-1777 | ATAAGCAUCCUCCCUCAGCGCCU | 6443 | 1755-1777 |
| AD-1707578.1 | CGCUGAGGGAGGAUGCUUAGU | 5960 | 1758-1778 | ACUAAGCAUCCTCCCUCAGCGCC | 6444 | 1756-1778 |
| AD-1707605.1 | GCACUAAGCCUAAGAAGUUCU | 5961 | 1811-1831 | AGAACUTCUUAGGCUUAGUGCCU | 6445 | 1809-1831 |
| AD-1707606.1 | CACUAAGCCUAAGAAGUUCCU | 5962 | 1812-1832 | AGGAACTUCUUAGGCUUAGUGCC | 6446 | 1810-1832 |
| AD-1707623.1 | ACAGGACCCACUGGGAGACAU | 5963 | 1849-1869 | ATGUCUCCCAGTGGGUCCUGUCC | 6447 | 1847-1869 |
| AD-1707624.1 | CAGGACCCACUGGGAGACAAU | 5964 | 1850-1870 | ATUGTCTCCCAGUGGGUCCUGUC | 6448 | 1848-1870 |
| AD-1707625.1 | AGGACCCACUGGGAGACAAGU | 5965 | 1851-1871 | ACUUGUCUCCCAGUGGGUCCUGU | 6449 | 1849-1871 |
| AD-1707626.1 | GGACCCACUGGGAGACAAGCU | 5966 | 1852-1872 | AGCUTGTCUCCCAGUGGGUCCUG | 6450 | 1850-1872 |
| AD-1707627.1 | GACCCACUGGGAGACAAGCAU | 5967 | 1853-1873 | ATGCTUGUCUCCCAGUGGGUCCU | 6451 | 1851-1873 |
| AD-1707628.1 | ACCCACUGGGAGACAAGCAUU | 5968 | 1854-1874 | AAUGCUTGUCUCCCAGUGGGUCC | 6452 | 1852-1874 |
| AD-1707629.1 | CCCACUGGGAGACAAGCAUUU | 5969 | 1855-1875 | AAAUGCTUGUCTCCCAGUGGGUC | 6453 | 1853-1875 |
| AD-1707630.1 | CCACUGGGAGACAAGCAUUUU | 5970 | 1856-1876 | AAAATGCUUGUCUCCCAGUGGGU | 6454 | 1854-1876 |
| AD-1707631.1 | CACUGGGAGACAAGCAUUUAU | 227 | 1857-1877 | ATAAAUGCUUGTCUCCCAGUGGG | 6455 | 1855-1877 |
| AD-1707632.1 | ACUGGGAGACAAGCAUUUAUU | 5971 | 1858-1878 | AAUAAATGCUUGUCUCCCAGUGG | 6456 | 1856-1878 |
| AD-1707633.1 | CUGGGAGACAAGCAUUUAUAU | 5972 | 1859-1879 | ATAUAAAUGCUTGUCUCCCAGUG | 6457 | 1857-1879 |
| AD-1707634.1 | UGGGAGACAAGCAUUUAUACU | 5973 | 1860-1880 | AGUATAAAUGCTUGUCUCCCAGU | 6458 | 1858-1880 |
| AD-1707636.1 | GGAGACAAGCAUUUAUACUUU | 5974 | 1862-1882 | AAAGTATAAAUGCUUGUCUCCCA | 6459 | 1860-1882 |
| AD-1707637.1 | GAGACAAGCAUUUAUACUUUU | 5975 | 1863-1883 | AAAAGUAUAAATGCUUGUCUCCC | 6460 | 1861-1883 |
| AD-1707638.1 | AGACAAGCAUUUAUACUUUCU | 5976 | 1864-1884 | AGAAAGTAUAAAUGCUUGUCUCC | 6461 | 1862-1884 |
| AD-1707639.1 | GACAAGCAUUUAUACUUUCUU | 72 | 1865-1885 | AAGAAAGUAUAAAUGCUUGUCUC | 64 | 1863-1885 |
| AD-1707640.1 | ACAAGCAUUUAUACUUUCUUU | 73 | 1866-1886 | AAAGAAAGUAUAAAUGCUUGUCU | 65 | 1864-1886 |
| AD-1707641.1 | CAAGCAUUUAUACUUUCUUUU | 5977 | 1867-1887 | AAAAGAAAGUATAAAUGCUUGUC | 6462 | 1865-1887 |
| AD-1708118.1 | CCAGACAUGAGCUGUGAGGGU | 5526 | 44373 | ACCCTCACAGCUCAUGUCUGGCU | 6463 | 44312 |
| AD-1708120.1 | AGACAUGAGCUGUGAGGGUCU | 5527 | 860-880 | AGACCCTCACAGCUCAUGUCUGG | 5980 | 858-880 |
| AD-1708124.1 | AUGAGCUGUGAGGGUCAAGCU | 5531 | 864-884 | AGCUTGACCCUCACAGCUCAUGU | 5984 | 862-884 |
| AD-1708126.1 | GAGCUGUGAGGGUCAAGCACU | 5533 | 866-886 | AGUGCUTGACCCUCACAGCUCAU | 5986 | 864-886 |
| AD-1708127.1 | AGCUGUGAGGGUCAAGCACAU | 5534 | 15-35 | AUGUGCTUGACCCUCACAGCUCA | 6464 | 13-35 |
| AD-1708130.1 | UGUGAGGGUCAAGCACAGCUU | 5537 | 870-890 | AAGCTGTGCUUGACCCUCACAGC | 5990 | 868-890 |
| AD-1708132.1 | UGAGGGUCAAGCACAGCUAUU | 5539 | 20-40 | AAUAGCTGUGCUUGACCCUCACA | 6465 | 18-40 |
| AD-1708135.1 | GGGUCAAGCACAGCUAUCCAU | 5541 | 23-43 | AUGGAUAGCUGUGCUUGACCCUC | 6466 | 21-43 |
| AD-1708140.1 | AAGCACAGCUAUCCAUCAGAU | 5543 | 28-48 | AUCUGATGGAUAGCUGUGCUUGA | 6467 | 26-48 |
| AD-1708143.1 | CACAGCUAUCCAUCAGAUGAU | 5545 | 31-51 | AUCATCTGAUGGAUAGCUGUGCU | 6468 | 29-51 |
| AD-1708145.1 | CAGCUAUCCAUCAGAUGAUCU | 5547 | 33-53 | AGAUCATCUGAUGGAUAGCUGUG | 6469 | 31-53 |
| AD-1708146.1 | AGCUAUCCAUCAGAUGAUCUU | 5548 | 886-906 | AAGATCAUCUGAUGGAUAGCUGU | 6001 | 884-906 |
| AD-1708148.1 | CUAUCCAUCAGAUGAUCUACU | 5550 | 36-56 | AGUAGATCAUCUGAUGGAUAGCU | 6470 | 34-56 |
| AD-1708149.1 | UAUCCAUCAGAUGAUCUACUU | 5551 | 37-57 | AAGUAGAUCAUCUGAUGGAUAGC | 6004 | 35-57 |
| AD-1708151.1 | UCCAUCAGAUGAUCUACUUUU | 5552 | 891-911 | AAAAGUAGAUCAUCUGAUGGAUA | 6006 | 889-911 |
| AD-1708152.1 | CCAUCAGAUGAUCUACUUUCU | 5553 | 892-912 | AGAAAGTAGAUCAUCUGAUGGAU | 6007 | 890-912 |
| AD-1708156.1 | CAGAUGAUCUACUUUCAGCCU | 5557 | 44-64 | AGGCTGAAAGUAGAUCAUCUGAU | 6011 | 42-64 |
| AD-1708158.1 | GAUGAUCUACUUUCAGCCUUU | 5559 | 46-66 | AAAGGCTGAAAGUAGAUCAUCUG | 6013 | 44-66 |
| AD-1708163.1 | UCUACUUUCAGCCUUCCUGAU | 5563 | 51-71 | AUCAGGAAGGCUGAAAGUAGAUC | 6471 | 49-71 |
| AD-1708166.1 | ACUUUCAGCCUUCCUGAGUCU | 5566 | 54-74 | AGACTCAGGAAGGCUGAAAGUAG | 6020 | 52-74 |
| AD-1708170.1 | UCAGCCUUCCUGAGUCCCAGU | 5567 | 910-930 | ACUGGGACUCAGGAAGGCUGAAA | 6021 | 908-930 |
| AD-1708220.1 | GUGGCAGUGGUGUCUGCUGUU | 136 | 120-140 | AACAGCAGACACCACUGCCACAC | 270 | 118-140 |
| AD-1708223.1 | GCAGUGGUGUCUGCUGUCACU | 5572 | 123-143 | AGUGACAGCAGACACCACUGCCA | 6026 | 121-143 |
| AD-1708225.1 | AGUGGUGUCUGCUGUCACUGU | 5574 | 125-145 | ACAGTGACAGCAGACACCACUGC | 6028 | 123-145 |
| AD-1708227.1 | UGGUGUCUGCUGUCACUGUGU | 5576 | 127-147 | ACACAGTGACAGCAGACACCACU | 6030 | 125-147 |
| AD-1708245.1 | GACUCAACAGACGGAGCAACU | 5580 | 170-190 | AGUUGCTCCGUCUGUUGAGUCUG | 6034 | 168-190 |
| AD-1708248.1 | UCAACAGACGGAGCAACUGCU | 5583 | 173-193 | AGCAGUTGCUCCGUCUGUUGAGU | 6037 | 171-193 |
| AD-1708249.1 | CAACAGACGGAGCAACUGCCU | 5584 | 1026-1046 | AGGCAGTUGCUCCGUCUGUUGAG | 6038 | 1024-1046 |
| AD-1708251.1 | ACAGACGGAGCAACUGCCAUU | 5586 | 176-196 | AAUGGCAGUUGCUCCGUCUGUUG | 6040 | 174-196 |
| AD-1708255.1 | ACGGAGCAACUGCCAUCCGAU | 139 | 180-200 | AUCGGATGGCAGUUGCUCCGUCU | 6472 | 178-200 |
| AD-1708260.1 | GCAACUGCCAUCCGAGGCUCU | 5593 | 185-205 | AGAGCCTCGGAUGGCAGUUGCUC | 6473 | 183-205 |
| AD-1708342.1 | CAAGCAGAACGAGCUCUGGUU | 146 | 339-359 | AACCAGAGCUCGUUCUGCUUGGG | 280 | 337-359 |
| AD-1708344.1 | AGCAGAACGAGCUCUGGUGCU | 5596 | 341-361 | AGCACCAGAGCUCGUUCUGCUUG | 6474 | 339-361 |
| AD-1708347.1 | AGAACGAGCUCUGGUGCUGGU | 5599 | 1196-1216 | ACCAGCACCAGAGCUCGUUCUGC | 6054 | 1194-1216 |
| AD-1708350.1 | ACGAGCUCUGGUGCUGGAGCU | 5602 | 347-367 | AGCUCCAGCACCAGAGCUCGUUC | 6057 | 345-367 |
| AD-1708356.1 | UCUGGUGCUGGAGCUAGCCAU | 5606 | 1205-1225 | AUGGCUAGCUCCAGCACCAGAGC | 6475 | 1203-1225 |
| AD-1708357.1 | CUGGUGCUGGAGCUAGCCAAU | 147 | 354-374 | AUUGGCTAGCUCCAGCACCAGAG | 6476 | 352-374 |
| AD-1708361.1 | UGCUGGAGCUAGCCAAGCAGU | 5610 | 358-378 | ACUGCUTGGCUAGCUCCAGCACC | 6066 | 356-378 |
| AD-1708365.1 | GGAGCUAGCCAAGCAGCAAAU | 5611 | 362-382 | AUUUGCTGCUUGGCUAGCUCCAG | 6477 | 360-382 |
| AD-1708368.1 | GCUAGCCAAGCAGCAAAUCCU | 5614 | 365-385 | AGGATUTGCUGCUUGGCUAGCUC | 6070 | 363-385 |
| AD-1708369.1 | CUAGCCAAGCAGCAAAUCCUU | 148 | 366-386 | AAGGAUTUGCUGCUUGGCUAGCU | 6071 | 364-386 |
| AD-1708397.1 | UGACCAGUCGUCCCAGAAUAU | 5621 | 1252-1272 | AUAUTCTGGGACGACUGGUCAGG | 6478 | 1250-1272 |
| AD-1708399.1 | ACCAGUCGUCCCAGAAUAACU | 66 | 402-422 | AGUUAUTCUGGGACGACUGGUCA | 57 | 400-422 |
| AD-1708401.1 | CAGUCGUCCCAGAAUAACUCU | 5624 | 1256-1276 | AGAGTUAUUCUGGGACGACUGGU | 6081 | 1254-1276 |
| AD-1708402.1 | AGUCGUCCCAGAAUAACUCAU | 5625 | 405-425 | AUGAGUTAUUCUGGGACGACUGG | 6479 | 403-425 |
| AD-1708403.1 | GUCGUCCCAGAAUAACUCAUU | 5626 | 406-426 | AAUGAGTUAUUCUGGGACGACUG | 6083 | 404-426 |
| AD-1708405.1 | CGUCCCAGAAUAACUCAUCCU | 5627 | 408-428 | AGGATGAGUUAUUCUGGGACGAC | 6480 | 406-428 |
| AD-1708407.1 | UCCCAGAAUAACUCAUCCUCU | 5629 | 410-430 | AGAGGATGAGUUAUUCUGGGACG | 6481 | 408-430 |
| AD-1708408.1 | CCCAGAAUAACUCAUCCUCCU | 5630 | 1263-1283 | AGGAGGAUGAGUUAUUCUGGGAC | 6482 | 1261-1283 |
| AD-1708447.1 | GGGAGUGUGGCUCCAGGGAAU | 5632 | 474-494 | AUUCCCTGGAGCCACACUCCCUG | 6483 | 472-494 |
| AD-1708454.1 | GAGGUCAUCAGCUUUGCUACU | 5638 | 501-521 | AGUAGCAAAGCUGAUGACCUCCU | 6484 | 499-521 |
| AD-1708457.1 | GUCAUCAGCUUUGCUACUGUU | 5641 | 504-524 | AACAGUAGCAAAGCUGAUGACCU | 6100 | 502-524 |
| AD-1708458.1 | UCAUCAGCUUUGCUACUGUCU | 5642 | 1357-1377 | AGACAGTAGCAAAGCUGAUGACC | 6101 | 1355-1377 |
| AD-1708462.1 | CAGCUUUGCUACUGUCACAGU | 5644 | 1361-1381 | ACUGTGACAGUAGCAAAGCUGAU | 6103 | 1359-1381 |
| AD-1708464.1 | GCUUUGCUACUGUCACAGACU | 5646 | 511-531 | AGUCTGTGACAGUAGCAAAGCUG | 6105 | 509-531 |
| AD-1708466.1 | UUUGCUACUGUCACAGACUCU | 5648 | 513-533 | AGAGTCTGUGACAGUAGCAAAGC | 6107 | 511-533 |
| AD-1708468.1 | UGCUACUGUCACAGACUCCAU | 5650 | 515-535 | AUGGAGTCUGUGACAGUAGCAAA | 6485 | 513-535 |
| AD-1708473.1 | CUGUCACAGACUCCACUUCAU | 68 | 520-540 | AUGAAGTGGAGUCUGUGACAGUA | 60 | 518-540 |
| AD-1708476.1 | UCACAGACUCCACUUCAGCCU | 5652 | 523-543 | AGGCTGAAGUGGAGUCUGUGACA | 6112 | 521-543 |
| AD-1708478.1 | ACAGACUCCACUUCAGCCUAU | 5654 | 525-545 | AUAGGCTGAAGUGGAGUCUGUGA | 6486 | 523-545 |
| AD-1708482.1 | ACUCCACUUCAGCCUACAGCU | 5658 | 529-549 | AGCUGUAGGCUGAAGUGGAGUCU | 6118 | 527-549 |
| AD-1708483.1 | CUCCACUUCAGCCUACAGCUU | 5659 | 530-550 | AAGCTGTAGGCUGAAGUGGAGUC | 6487 | 528-550 |
| AD-1708485.1 | CCACUUCAGCCUACAGCUCCU | 5660 | 532-552 | AGGAGCTGUAGGCUGAAGUGGAG | 6120 | 530-552 |
| AD-1708559.1 | UGGCACUCUUUGCUUGAGGAU | 5675 | 635-655 | AUCCTCAAGCAAAGAGUGCCAGG | 6488 | 633-655 |
| AD-1708561.1 | GCACUCUUUGCUUGAGGAUCU | 5677 | 637-657 | AGAUCCTCAAGCAAAGAGUGCCA | 6137 | 635-657 |
| AD-1708564.1 | CUCUUUGCUUGAGGAUCUUCU | 162 | 640-660 | AGAAGATCCUCAAGCAAAGAGUG | 6139 | 638-660 |
| AD-1708565.1 | UCUUUGCUUGAGGAUCUUCCU | 5679 | 641-661 | AGGAAGAUCCUCAAGCAAAGAGU | 6140 | 639-661 |
| AD-1708567.1 | UUUGCUUGAGGAUCUUCCGAU | 5680 | 643-663 | AUCGGAAGAUCCUCAAGCAAAGA | 6489 | 641-663 |
| AD-1708647.1 | UGGCUGAGCACCACAUCACCU | 5685 | 1555-1575 | AGGUGATGUGGUGCUCAGCCAGG | 6490 | 1553-1575 |
| AD-1708648.1 | GGCUGAGCACCACAUCACCAU | 5686 | 1556-1576 | AUGGTGAUGUGGUGCUCAGCCAG | 6491 | 1554-1576 |
| AD-1708650.1 | CUGAGCACCACAUCACCAACU | 5688 | 706-726 | AGUUGGTGAUGUGGUGCUCAGCC | 6492 | 704-726 |
| AD-1708657.1 | CCACAUCACCAACCUGGGCUU | 5692 | 713-733 | AAGCCCAGGUUGGUGAUGUGGUG | 6154 | 711-733 |
| AD-1708662.1 | UCACCAACCUGGGCUGGCAUU | 5696 | 1570-1590 | AAUGCCAGCCCAGGUUGGUGAUG | 6158 | 1568-1590 |
| AD-1708680.1 | CCUUAACUCUGCCCUCUAGUU | 5699 | 739-759 | AACUAGAGGGCAGAGUUAAGGUA | 6161 | 737-759 |
| AD-1708683.1 | UAACUCUGCCCUCUAGUGGCU | 5702 | 742-762 | AGCCACTAGAGGGCAGAGUUAAG | 6164 | 740-762 |
| AD-1708697.1 | GUCUGGUGUCCUGAAACUGCU | 5705 | 1628-1648 | AGCAGUTUCAGGACACCAGACUU | 6167 | 1626-1648 |
| AD-1708698.1 | UCUGGUGUCCUGAAACUGCAU | 5706 | 1629-1649 | AUGCAGTUUCAGGACACCAGACU | 6493 | 1627-1649 |
| AD-1708700.1 | UGGUGUCCUGAAACUGCAACU | 5708 | 779-799 | AGUUGCAGUUUCAGGACACCAGA | 6170 | 777-799 |
| AD-1708704.1 | GUCCUGAAACUGCAACUAGAU | 169 | 783-803 | AUCUAGTUGCAGUUUCAGGACAC | 6494 | 781-803 |
| AD-1708706.1 | CCUGAAACUGCAACUAGACUU | 5713 | 785-805 | AAGUCUAGUUGCAGUUUCAGGAC | 6176 | 783-805 |
| AD-1708707.1 | CUGAAACUGCAACUAGACUGU | 5714 | 786-806 | ACAGTCTAGUUGCAGUUUCAGGA | 6177 | 784-806 |
| AD-1708709.1 | GAAACUGCAACUAGACUGCAU | 5716 | 788-808 | AUGCAGTCUAGUUGCAGUUUCAG | 6495 | 786-808 |
| AD-1708711.1 | AACUGCAACUAGACUGCAGAU | 5718 | 790-810 | AUCUGCAGUCUAGUUGCAGUUUC | 6496 | 788-810 |
| AD-1708719.1 | GCAACAGCACAGUUACUGGAU | 5721 | 820-840 | AUCCAGTAACUGUGCUGUUGCCU | 6497 | 818-840 |
| AD-1708721.1 | AACAGCACAGUUACUGGACAU | 5723 | 1677-1697 | AUGUCCAGUAACUGUGCUGUUGC | 6498 | 1675-1697 |
| AD-1708724.1 | AGCACAGUUACUGGACAACCU | 5725 | 825-845 | AGGUTGTCCAGUAACUGUGCUGU | 6499 | 823-845 |
| AD-1708746.1 | UUGGACACAGCAGGACACCAU | 5727 | 855-875 | AUGGTGTCCUGCUGUGUCCAAGA | 6500 | 853-875 |
| AD-1708748.1 | GGACACAGCAGGACACCAGCU | 5729 | 1712-1732 | AGCUGGTGUCCUGCUGUGUCCAA | 6501 | 1710-1732 |
| AD-1708751.1 | CACAGCAGGACACCAGCAGCU | 5732 | 860-880 | AGCUGCTGGUGUCCUGCUGUGUC | 6502 | 858-880 |
| AD-1708759.1 | GACACCAGCAGCCCUUCCUAU | 173 | 868-888 | AUAGGAAGGGCUGCUGGUGUCCU | 308 | 866-888 |
| AD-1708763.1 | CCAGCAGCCCUUCCUAGAGCU | 5737 | 872-892 | AGCUCUAGGAAGGGCUGCUGGUG | 6202 | 870-892 |
| AD-1708764.1 | CAGCAGCCCUUCCUAGAGCUU | 5738 | 873-893 | AAGCTCTAGGAAGGGCUGCUGGU | 6203 | 871-893 |
| AD-1708766.1 | GCAGCCCUUCCUAGAGCUUAU | 5739 | 1730-1750 | AUAAGCTCUAGGAAGGGCUGCUG | 6503 | 1728-1750 |
| AD-1708771.1 | CCUUCCUAGAGCUUAAGAUCU | 174 | 880-900 | AGAUCUTAAGCUCUAGGAAGGGC | 6504 | 878-900 |
| AD-1708813.1 | UUACGUAGACUUCCAGGAACU | 5744 | 986-1006 | AGUUCCTGGAAGUCUACGUAAUG | 6210 | 984-1006 |
| AD-1708819.1 | AGACUUCCAGGAACUGGGAUU | 5748 | 992-1012 | AAUCCCAGUUCCUGGAAGUCUAC | 6215 | 990-1012 |
| AD-1708842.1 | GUACCAGCUGAAUUACUGCAU | 5755 | 1040-1060 | AUGCAGTAAUUCAGCUGGUACCC | 6505 | 1038-1060 |
| AD-1708844.1 | ACCAGCUGAAUUACUGCAGUU | 5756 | 1042-1062 | AACUGCAGUAAUUCAGCUGGUAC | 6506 | 1040-1062 |
| AD-1708849.1 | CUGAAUUACUGCAGUGGGCAU | 5761 | 1047-1067 | AUGCCCACUGCAGUAAUUCAGCU | 6507 | 1045-1067 |
| AD-1708856.1 | ACUGCAGUGGGCAGUGCCCUU | 182 | 1054-1074 | AAGGGCACUGCCCACUGCAGUAA | 317 | 1052-1074 |
| AD-1708872.1 | CAGGCAUUGCUGCCUCUUUCU | 183 | 1093-1113 | AGAAAGAGGCAGCAAUGCCUGGG | 318 | 1091-1113 |
| AD-1708875.1 | CAUUGCUGCCUCUUUCCAUUU | 5767 | 1097-1117 | AAAUGGAAAGAGGCAGCAAUGCC | 6235 | 1095-1117 |
| AD-1708882.1 | GCCUCUUUCCAUUCUGCCGUU | 5774 | 1104-1124 | AACGGCAGAAUGGAAAGAGGCAG | 6242 | 1102-1124 |
| AD-1708887.1 | UUUCCAUUCUGCCGUCUUCAU | 184 | 1109-1129 | AUGAAGACGGCAGAAUGGAAAGA | 319 | 1107-1129 |
| AD-1708889.1 | UCCAUUCUGCCGUCUUCAGCU | 5780 | 1111-1131 | AGCUGAAGACGGCAGAAUGGAAA | 6249 | 1109-1131 |
| AD-1708890.1 | CCAUUCUGCCGUCUUCAGCCU | 5781 | 1112-1132 | AGGCTGAAGACGGCAGAAUGGAA | 6250 | 1110-1132 |
| AD-1708899.1 | CGUCUUCAGCCUCCUCAAAGU | 185 | 1121-1141 | ACUUTGAGGAGGCUGAAGACGGC | 6254 | 1119-1141 |
| AD-1708901.1 | UCUUCAGCCUCCUCAAAGCCU | 5786 | 1978-1998 | AGGCTUTGAGGAGGCUGAAGACG | 6256 | 1976-1998 |
| AD-1708902.1 | CUUCAGCCUCCUCAAAGCCAU | 5787 | 1979-1999 | AUGGCUTUGAGGAGGCUGAAGAC | 6508 | 1977-1999 |
| AD-1708907.1 | GCCUCCUCAAAGCCAACAAUU | 5791 | 1129-1149 | AAUUGUTGGCUUUGAGGAGGCUG | 6509 | 1127-1149 |
| AD-1708908.1 | CCUCCUCAAAGCCAACAAUCU | 5792 | 1130-1150 | AGAUTGTUGGCUUUGAGGAGGCU | 6510 | 1128-1150 |
| AD-1708911.1 | CCUCAAAGCCAACAAUCCUUU | 186 | 1133-1153 | AAAGGATUGUUGGCUUUGAGGAG | 6265 | 1131-1153 |
| AD-1708921.1 | AACAAUCCUUGGCCUGCCAGU | 5800 | 1143-1163 | ACUGGCAGGCCAAGGAUUGUUGG | 6271 | 1141-1163 |
| AD-1708956.1 | UCUCCUCUACCUGGAUCAUAU | 5811 | 2057-2077 | AUAUGATCCAGGUAGAGGAGAGA | 6511 | 2055-2077 |
| AD-1708957.1 | CUCCUCUACCUGGAUCAUAAU | 5812 | 1203-1223 | AUUATGAUCCAGGUAGAGGAGAG | 6512 | 1201-1223 |
| AD-1708962.1 | CUACCUGGAUCAUAAUGGCAU | 5817 | 2063-2083 | AUGCCATUAUGAUCCAGGUAGAG | 6513 | 2061-2083 |
| AD-1708963.1 | UACCUGGAUCAUAAUGGCAAU | 191 | 1209-1229 | AUUGCCAUUAUGAUCCAGGUAGA | 326 | 1207-1229 |
| AD-1708971.1 | UCAUAAUGGCAAUGUGGUCAU | 5825 | 2072-2092 | AUGACCACAUUGCCAUUAUGAUC | 6514 | 2070-2092 |
| AD-1708974.1 | UAAUGGCAAUGUGGUCAAGAU | 5828 | 1220-1240 | AUCUTGACCACAUUGCCAUUAUG | 6515 | 1218-1240 |
| AD-1708976.1 | AUGGCAAUGUGGUCAAGACGU | 5829 | 1222-1242 | ACGUCUTGACCACAUUGCCAUUA | 6303 | 1220-1242 |
| AD-1708977.1 | UGGCAAUGUGGUCAAGACGGU | 5830 | 2078-2098 | ACCGTCTUGACCACAUUGCCAUU | 6304 | 2076-2098 |
| AD-1708984.1 | GUGGUCAAGACGGAUGUGCCU | 5837 | 2085-2105 | AGGCACAUCCGUCUUGACCACAU | 6516 | 2083-2105 |
| AD-1708986.1 | GGUCAAGACGGAUGUGCCAGU | 5839 | 2087-2107 | ACUGGCACAUCCGUCUUGACCAC | 6313 | 2085-2107 |
| AD-1708990.1 | AAGACGGAUGUGCCAGAUAUU | 193 | 1236-1256 | AAUATCTGGCACAUCCGUCUUGA | 6317 | 1234-1256 |
| AD-1709009.1 | UGGUGGUGGAGGCCUGUGGCU | 5853 | 1255-1275 | AGCCACAGGCCUCCACCACCAUA | 6517 | 1253-1275 |
| AD-1709021.1 | CCUGUGGCUGCAGCUAGCAAU | 5856 | 1267-1287 | AUUGCUAGCUGCAGCCACAGGCC | 6518 | 1265-1287 |
| AD-1709022.1 | CUGUGGCUGCAGCUAGCAAGU | 5857 | 2123-2143 | ACUUGCTAGCUGCAGCCACAGGC | 6333 | 2121-2143 |
| AD-1709026.1 | GGCUGCAGCUAGCAAGAGGAU | 5861 | 1272-1292 | AUCCTCTUGCUAGCUGCAGCCAC | 6519 | 1270-1292 |
| AD-1709028.1 | CUGCAGCUAGCAAGAGGACCU | 5863 | 1274-1294 | AGGUCCTCUUGCUAGCUGCAGCC | 6339 | 1272-1294 |
| AD-1709031.1 | CAGCUAGCAAGAGGACCUGGU | 5865 | 1277-1297 | ACCAGGTCCUCUUGCUAGCUGCA | 6520 | 1275-1297 |
| AD-1709032.1 | GCUUUGGAGUGAAGAGACCAU | 5866 | 2153-2173 | AUGGTCTCUUCACUCCAAAGCCC | 6521 | 2151-2173 |
| AD-1709034.1 | UUUGGAGUGAAGAGACCAAGU | 5867 | 2155-2175 | ACUUGGTCUCUUCACUCCAAAGC | 6522 | 2153-2175 |
| AD-1709038.1 | GAGUGAAGAGACCAAGAUGAU | 5870 | 2159-2179 | AUCATCTUGGUCUCUUCACUCCA | 6523 | 2157-2179 |
| AD-1709041.1 | UGAAGAGACCAAGAUGAAGUU | 5871 | 1307-1327 | AACUTCAUCUUGGUCUCUUCACU | 6349 | 1305-1327 |
| AD-1709044.1 | AGAGACCAAGAUGAAGUUUCU | 198 | 1310-1330 | AGAAACTUCAUCUUGGUCUCUUC | 6351 | 1308-1330 |
| AD-1709052.1 | AGAUGAAGUUUCCCAGGCACU | 5880 | 2173-2193 | AGUGCCTGGGAAACUUCAUCUUG | 6359 | 2171-2193 |
| AD-1709056.1 | GAAGUUUCCCAGGCACAGGGU | 5883 | 1322-1342 | ACCCTGTGCCUGGGAAACUUCAU | 6362 | 1320-1342 |
| AD-1709090.1 | ACCCAACAACCACCUGGCAAU | 201 | 1383-1403 | AUUGCCAGGUGGUUGUUGGGUUG | 336 | 1381-1403 |
| AD-1709097.1 | AACCACCUGGCAAUAUGACUU | 5897 | 1390-1410 | AAGUCATAUUGCCAGGUGGUUGU | 6377 | 1388-1410 |
| AD-1709098.1 | ACCACCUGGCAAUAUGACUCU | 5898 | 2246-2266 | AGAGTCAUAUUGCCAGGUGGUUG | 6378 | 2244-2266 |
| AD-1709100.1 | CACCUGGCAAUAUGACUCACU | 5900 | 1393-1413 | AGUGAGTCAUAUUGCCAGGUGGU | 6524 | 1391-1413 |
| AD-1709102.1 | CCUGGCAAUAUGACUCACUUU | 5902 | 1395-1415 | AAAGTGAGUCAUAUUGCCAGGUG | 6525 | 1393-1415 |
| AD-1709104.1 | UGGCAAUAUGACUCACUUGAU | 5904 | 1397-1417 | AUCAAGTGAGUCAUAUUGCCAGG | 6526 | 1395-1417 |
| AD-1709106.1 | GCAAUAUGACUCACUUGACCU | 5905 | 1399-1419 | AGGUCAAGUGAGUCAUAUUGCCA | 6386 | 1397-1419 |
| AD-1709111.1 | GGACCCAAAUGGGCACUUUCU | 5906 | 1425-1445 | AGAAAGTGCCCAUUUGGGUCCCA | 6387 | 1423-1445 |
| AD-1709120.1 | UGGGCACUUUCUUGUCUGAGU | 5913 | 2289-2309 | ACUCAGACAAGAAAGUGCCCAUU | 6394 | 2287-2309 |
| AD-1709122.1 | GGCACUUUCUUGUCUGAGACU | 5915 | 1436-1456 | AGUCTCAGACAAGAAAGUGCCCA | 6396 | 1434-1456 |
| AD-1709124.1 | CACUUUCUUGUCUGAGACUCU | 5917 | 1438-1458 | AGAGTCTCAGACAAGAAAGUGCC | 6398 | 1436-1458 |
| AD-1709126.1 | CUUUCUUGUCUGAGACUCUGU | 5918 | 1440-1460 | ACAGAGTCUCAGACAAGAAAGUG | 6399 | 1438-1460 |
| AD-1709128.1 | UUCUUGUCUGAGACUCUGGCU | 5920 | 1442-1462 | AGCCAGAGUCUCAGACAAGAAAG | 6401 | 1440-1462 |
| AD-1709130.1 | CUUGUCUGAGACUCUGGCUUU | 5922 | 2299-2319 | AAAGCCAGAGUCUCAGACAAGAA | 6403 | 2297-2319 |
| AD-1709148.1 | UAUUCCAGGUUGGCUGAUGUU | 5923 | 1463-1483 | AACATCAGCCAACCUGGAAUAAG | 6404 | 1461-1483 |
| AD-1709151.1 | UCCAGGUUGGCUGAUGUGUUU | 206 | 1466-1486 | AAACACAUCAGCCAACCUGGAAU | 341 | 1464-1486 |
| AD-1709201.1 | CUCUCUCCUCUACCUGGAUCU | 5808 | 1199-1219 | AGAUCCAGGUAGAGGAGAGAGAG | 6280 | 1197-1219 |
| AD-1709237.1 | GAUUUCCUGCCCUAAGUCCUU | 210 | 1534-1554 | AAGGACTUAGGGCAGGAAAUCAU | 6409 | 1532-1554 |
| AD-1709239.1 | UUUCCUGCCCUAAGUCCUGUU | 5928 | 1536-1556 | AACAGGACUUAGGGCAGGAAAUC | 6410 | 1534-1556 |
| AD-1709253.1 | AGAAGAUGUCAGGGACUAGGU | 5929 | 1557-1577 | ACCUAGTCCCUGACAUCUUCUCA | 6411 | 1555-1577 |
| AD-1709254.1 | AAGAUGUCAGGGACUAGGGAU | 5931 | 1559-1579 | AUCCCUAGUCCCUGACAUCUUCU | 6527 | 1557-1579 |
| AD-1709298.1 | AGGAGGAAGCAGAUAGAUGGU | 5936 | 1647-1667 | ACCATCTAUCUGCUUCCUCCUCC | 6418 | 1645-1667 |
| AD-1709300.1 | GAGGAAGCAGAUAGAUGGUCU | 5937 | 1649-1669 | AGACCATCUAUCUGCUUCCUCCU | 6420 | 1647-1669 |
| AD-1709301.1 | AGGAAGCAGAUAGAUGGUCCU | 5938 | 2499-2519 | AGGACCAUCUAUCUGCUUCCUCC | 6528 | 2497-2519 |
| AD-1709304.1 | AAGCAGAUAGAUGGUCCAGCU | 5941 | 2502-2522 | AGCUGGACCAUCUAUCUGCUUCC | 6424 | 2500-2522 |
| AD-1709307.1 | CAGAUAGAUGGUCCAGCAGGU | 5944 | 1656-1676 | ACCUGCTGGACCAUCUAUCUGCU | 6427 | 1654-1676 |
| AD-1709310.1 | AUAGAUGGUCCAGCAGGCUUU | 5947 | 1659-1679 | AAAGCCTGCUGGACCAUCUAUCU | 6430 | 1657-1679 |
| AD-1709314.1 | AUGGUCCAGCAGGCUUGAAGU | 5951 | 1663-1683 | ACUUCAAGCCUGCUGGACCAUCU | 6434 | 1661-1683 |
| AD-1709315.1 | UGGUCCAGCAGGCUUGAAGCU | 5952 | 2513-2533 | AGCUTCAAGCCUGCUGGACCAUC | 6529 | 2511-2533 |
| AD-1709317.1 | GUCCAGCAGGCUUGAAGCAGU | 5954 | 1666-1686 | ACUGCUTCAAGCCUGCUGGACCA | 6437 | 1664-1686 |
| AD-1709372.1 | GGUCAAGAGGGAGAUGGGCAU | 5957 | 2584-2604 | AUGCCCAUCUCCCUCUUGACCUU | 6530 | 2582-2604 |
| AD-1709390.1 | GCGCUGAGGGAGGAUGCUUAU | 5959 | 1757-1777 | AUAAGCAUCCUCCCUCAGCGCCU | 6531 | 1755-1777 |
| AD-1709410.1 | GCACUAAGCCUAAGAAGUUCU | 5961 | 1811-1831 | AGAACUTCUUAGGCUUAGUGCCU | 6445 | 1809-1831 |
| AD-1709411.1 | CACUAAGCCUAAGAAGUUCCU | 5962 | 1812-1832 | AGGAACTUCUUAGGCUUAGUGCC | 6446 | 1810-1832 |
| AD-1709429.1 | CAGGACCCACUGGGAGACAAU | 5964 | 1850-1870 | AUUGTCTCCCAGUGGGUCCUGUC | 6532 | 1848-1870 |
| AD-1709431.1 | GGACCCACUGGGAGACAAGCU | 5966 | 2701-2721 | AGCUTGTCUCCCAGUGGGUCCUG | 6450 | 2699-2721 |
| AD-1709433.1 | ACCCACUGGGAGACAAGCAUU | 5968 | 1854-1874 | AAUGCUTGUCUCCCAGUGGGUCC | 6452 | 1852-1874 |
| AD-1709434.1 | CCCACUGGGAGACAAGCAUUU | 5969 | 1855-1875 | AAAUGCTUGUCUCCCAGUGGGUC | 6533 | 1853-1875 |
| AD-1709442.1 | GAGACAAGCAUUUAUACUUUU | 5975 | 2712-2732 | AAAAGUAUAAAUGCUUGUCUCCC | 6534 | 2710-2732 |
| AD-1709443.1 | AGACAAGCAUUUAUACUUUCU | 5976 | 1864-1884 | AGAAAGTAUAAAUGCUUGUCUCC | 6461 | 1862-1884 |
| AD-1709502.1 | AGGUCAAGAGGGAGAUGGGCU | 5956 | 1734-1754 | AGCCCATCUCCCUCUUGACCUUC | 6439 | 1732-1754 |
| AD-1711741.1 | CAAGCACAGCUAUCCAUCAGU | 5542 | 27-47 | ACUGAUGGAUAGCUGUGCUUGCU | 6535 | 25-47 |
| AD-1711742.1 | AUCUACUUUCAGCCUUCCUGU | 5562 | 50-70 | ACAGGAAGGCUGAAAGUAGAUCU | 6536 | 48-70 |
| AD-1711743.1 | AGCCAAGCAGCAAAUCCUGGU | 5616 | 368-388 | ACCAGGAUUUGCUGCUUGGCUCU | 6537 | 366-388 |
| AD-1711744.1 | ACCAGUCGUCCCAGAAUAACU | 66 | 402-422 | AGUUAUTCUGGGACGACUGGUCU | 58 | 400-422 |
| AD-1711745.1 | CCUGGCACUCUUUGCUUGAGU | 5673 | 633-653 | ACUCAAGCAAAGAGUGCCAGGCU | 6538 | 631-653 |
| AD-1711746.1 | CUGGCACUCUUUGCUUGAGGU | 5674 | 634-654 | ACCUCAAGCAAAGAGUGCCAGCU | 6539 | 632-654 |
| AD-1711747.1 | CUUUGCUUGAGGAUCUUCCGU | 70 | 642-662 | ACGGAAGAUCCTCAAGCAAAGCU | 6540 | 640-662 |
| AD-1711748.1 | UUUGCUUGAGGAUCUUCCGAU | 5680 | 643-663 | ATCGGAAGAUCCUCAAGCAAACU | 6541 | 641-663 |
| AD-1711749.1 | UUGCUUGAGGAUCUUCCGAUU | 5681 | 644-664 | AAUCGGAAGAUCCUCAAGCAACU | 6542 | 642-664 |
| AD-1711750.1 | UGCUUGAGGAUCUUCCGAUGU | 5682 | 645-665 | ACAUCGGAAGATCCUCAAGCACU | 6543 | 643-665 |
| AD-1711751.1 | GCUUGAGGAUCUUCCGAUGGU | 5683 | 646-666 | ACCATCGGAAGAUCCUCAAGCCU | 6544 | 644-666 |
| AD-1711752.1 | AGCACCACAUCACCAACCUGU | 5690 | 709-729 | ACAGGUTGGUGAUGUGGUGCUCU | 6545 | 707-729 |
| AD-1711753.1 | ACGUAGACUUCCAGGAACUGU | 5745 | 988-1008 | ACAGTUCCUGGAAGUCUACGUCU | 6546 | 986-1008 |
| AD-1711754.1 | CGUAGACUUCCAGGAACUGGU | 5746 | 989-1009 | ACCAGUTCCUGGAAGUCUACGCU | 6547 | 987-1009 |
| AD-1711755.1 | GACUUCCAGGAACUGGGAUGU | 5749 | 993-1013 | ACAUCCCAGUUCCUGGAAGUCCU | 6548 | 991-1013 |
| AD-1711756.1 | UGCUGCCUCUUUCCAUUCUGU | 5770 | 1100-1120 | ACAGAATGGAAAGAGGCAGCACU | 6549 | 1098-1120 |
| AD-1711757.1 | GCUGCCUCUUUCCAUUCUGCU | 5771 | 1101-1121 | AGCAGAAUGGAAAGAGGCAGCCU | 6550 | 1099-1121 |
| AD-1711758.1 | CUGCCUCUUUCCAUUCUGCCU | 5772 | 1102-1122 | AGGCAGAAUGGAAAGAGGCAGCU | 6551 | 1100-1122 |
| AD-1711759.1 | UUCCAUUCUGCCGUCUUCAGU | 5779 | 1110-1130 | ACUGAAGACGGCAGAAUGGAACU | 6552 | 1108-1130 |
| AD-1711760.1 | CCUCAAAGCCAACAAUCCUUU | 186 | 1133-1153 | AAAGGATUGUUGGCUUUGAGGCU | 6553 | 1131-1153 |
| AD-1711761.1 | CUCAAAGCCAACAAUCCUUGU | 5795 | 1134-1154 | ACAAGGAUUGUTGGCUUUGAGCU | 6554 | 1132-1154 |
| AD-1711762.1 | UGUGUCCCUACUGCCCGAAGU | 5805 | 1173-1193 | ACUUCGGGCAGTAGGGACACACU | 6555 | 1171-1193 |
| AD-1711763.1 | GUGUCCCUACUGCCCGAAGGU | 189 | 1174-1194 | ACCUTCGGGCAGUAGGGACACCU | 6556 | 1172-1194 |
| AD-1711764.1 | CCUCUACCUGGAUCAUAAUGU | 5814 | 1205-1225 | ACAUTATGAUCCAGGUAGAGGCU | 6557 | 1203-1225 |
| AD-1711765.1 | CUCUACCUGGAUCAUAAUGGU | 5815 | 1206-1226 | ACCATUAUGAUCCAGGUAGAGCU | 6558 | 1204-1226 |
| AD-1711766.1 | GGAUCAUAAUGGCAAUGUGGU | 5822 | 1214-1234 | ACCACATUGCCAUUAUGAUCCCU | 6559 | 1212-1234 |
| AD-1711767.1 | GAUCAUAAUGGCAAUGUGGUU | 5823 | 1215-1235 | AACCACAUUGCCAUUAUGAUCCU | 6560 | 1213-1235 |
| AD-1711768.1 | AUGGCAAUGUGGUCAAGACGU | 5829 | 1222-1242 | ACGUCUTGACCACAUUGCCAUCU | 6561 | 1220-1242 |
| AD-1711769.1 | GGCAAUGUGGUCAAGACGGAU | 5831 | 1224-1244 | ATCCGUCUUGACCACAUUGCCCU | 6562 | 1222-1244 |
| AD-1711770.1 | GCAAUGUGGUCAAGACGGAUU | 5832 | 1225-1245 | AAUCCGTCUUGACCACAUUGCCU | 6563 | 1223-1245 |
| AD-1711771.1 | UGCCAGAUAUGGUGGUGGAGU | 5851 | 1246-1266 | ACUCCACCACCAUAUCUGGCACU | 6564 | 1244-1266 |
| AD-1711772.1 | UGGCUGCAGCUAGCAAGAGGU | 5860 | 1271-1291 | ACCUCUTGCUAGCUGCAGCCACU | 6565 | 1269-1291 |
| AD-1711773.1 | GGAGUGAAGAGACCAAGAUGU | 5869 | 1303-1323 | ACAUCUTGGUCTCUUCACUCCCU | 6566 | 1301-1323 |
| AD-1711774.1 | AAGAGACCAAGAUGAAGUUUU | 5872 | 1309-1329 | AAAACUTCAUCTUGGUCUCUUCU | 6567 | 1307-1329 |
| AD-1711775.1 | UUCCAGGUUGGCUGAUGUGUU | 5925 | 1465-1485 | AACACATCAGCCAACCUGGAACU | 6568 | 1463-1485 |
| AD-1711776.1 | CCAGGUUGGCUGAUGUGUUGU | 5926 | 1467-1487 | ACAACACAUCAGCCAACCUGGCU | 6569 | 1465-1487 |
| AD-1711777.1 | CAGGUUGGCUGAUGUGUUGGU | 5927 | 1468-1488 | ACCAACACAUCAGCCAACCUGCU | 6570 | 1466-1488 |
| AD-1711778.1 | GUCCAGCAGGCUUGAAGCAGU | 5954 | 1666-1686 | ACUGCUTCAAGCCUGCUGGACCU | 6571 | 1664-1686 |
| AD-1711779.1 | GGAGACAAGCAUUUAUACUUU | 5974 | 1862-1882 | AAAGTATAAAUGCUUGUCUCCCU | 6572 | 1860-1882 |

TABLE 20
Modified Sense and Antisense Strand Sequences of INHBE dsRNA Agents

| | | SEQ | | SEQ | | SEQ |
|---|---|---|---|---|---|---|
| | | ID | | ID | | ID |
| Duplex Name | Sense Strand Sequence 5′ to 3′ | NO. | Antisense Strand Sequence 5′ to 3′ | NO. | mRNA target sequence | NO. |
| AD-1706265.1 | gscscagacaUfGfAfgcugugagguL96 | 6573 | asdCscudCadCagcudCaUfgucuggcsusa | 7233 | UAGCCAGACAUGAGCUGUGAGGG | 7944 |
| AD-1706266.1 | cscsagacauGfAfGfcugugaggguL96 | 6574 | asdCsccdTcdAcagcdTcAfugucuggscsu | 7234 | AGCCAGACAUGAGCUGUGAGGGU | 7945 |
| AD-1706268.1 | asgsacaugaGfCfUfgugagggucuL96 | 6575 | asdGsacdCcdTcacadGcUfcaugucusgsg | 7235 | CCAGACAUGAGCUGUGAGGGUCA | 7946 |
| AD-1706269.1 | gsascaugagCfUfGfugagggucauL96 | 6576 | asdTsgadCcdCucacdAgCfucaugucsusg | 7236 | CAGACAUGAGCUGUGAGGGUCAA | 7947 |
| AD-1706270.1 | ascsaugagcUfGfUfgagggucaauL96 | 6577 | asdTsugdAcdCcucadCaGfcucauguscsu | 7237 | AGACAUGAGCUGUGAGGGUCAAG | 7948 |
| AD-1706271.1 | csasugagcuGfUfGfagggucaaguL96 | 6578 | asdCsuudGadCccucdAcAfgcucaugsusc | 7238 | GACAUGAGCUGUGAGGGUCAAGC | 7949 |
| AD-1706272.1 | asusgagcugUfGfAfgggucaagcuL96 | 6579 | asdGscudTgdAcccudCaCfagcucausgsu | 7239 | ACAUGAGCUGUGAGGGUCAAGCA | 7950 |
| AD-1706273.1 | usgsagcuguGfAfGfggucaagcauL96 | 6580 | asdTsgcdTudGacccdTcAfcagcucasusg | 7240 | CAUGAGCUGUGAGGGUCAAGCAC | 7951 |
| AD-1706274.1 | gsasgcugugAfGfGfgucaagcacuL96 | 6581 | asdGsugdCudTgaccdCuCfacagcucsasu | 7241 | AUGAGCUGUGAGGGUCAAGCACA | 7952 |
| AD-1706275.1 | asgscugugaGfGfGfucaagcacauL96 | 6582 | asdTsgudGcdTugacdCcUfcacagcuscsa | 7242 | UGAGCUGUGAGGGUCAAGCACAG | 7953 |
| AD-1706276.1 | gscsugugagGfGfUfcaagcacaguL96 | 6583 | asdCsugdTgdCuugadCcCfucacagcsusc | 7243 | GAGCUGUGAGGGUCAAGCACAGC | 7954 |
| AD-1706277.1 | csusgugaggGfUfCfaagcacagcuL96 | 6584 | asdGscudGudGcuugdAcCfcucacagscsu | 7244 | AGCUGUGAGGGUCAAGCACAGCU | 7955 |
| AD-1706278.1 | usgsugagggUfCfAfagcacagcuuL96 | 6585 | asdAsgcdTgdTgcuudGaCfccucacasgsc | 7245 | GCUGUGAGGGUCAAGCACAGCUA | 7956 |
| AD-1706279.1 | gsusgaggguCfAfAfgcacagcuauL96 | 6586 | asdTsagdCudGugcudTgAfcccucacsasg | 7246 | CUGUGAGGGUCAAGCACAGCUAU | 7957 |
| AD-1706280.1 | usgsagggucAfAfGfcacagcuauuL96 | 6587 | asdAsuadGcdTgugcdTuGfacccucascsa | 7247 | UGUGAGGGUCAAGCACAGCUAUC | 7958 |
| AD-1706281.1 | gsasgggucaAfGfCfacagcuaucuL96 | 6588 | asdGsaudAgdCugugdCuUfgacccucsasc | 7248 | GUGAGGGUCAAGCACAGCUAUCC | 7959 |
| AD-1706282.1 | asgsggucaaGfCfAfcagcuauccuL96 | 6589 | asdGsgadTadGcugudGcUfugacccuscsa | 7249 | UGAGGGUCAAGCACAGCUAUCCA | 7960 |
| AD-1706283.1 | gsgsgucaagCfAfCfagcuauccauL96 | 6590 | asdTsggdAudAgcugdTgCfuugacccsusc | 7250 | GAGGGUCAAGCACAGCUAUCCAU | 7961 |
| AD-1706287.1 | csasagcacaGfCfUfauccaucaguL96 | 6591 | asdCsugdAudGgauadGcUfgugcuugsasc | 7251 | GUCAAGCACAGCUAUCCAUCAGA | 7962 |
| AD-1706288.1 | asasgcacagCfUfAfuccaucagauL96 | 6592 | asdTscudGadTggaudAgCfugugcuusgsa | 7252 | UCAAGCACAGCUAUCCAUCAGAU | 7963 |
| AD-1706290.1 | gscsacagcuAfUfCfcaucagauguL96 | 6593 | asdCsaudCudGauggdAuAfgcugugcsusu | 7253 | AAGCACAGCUAUCCAUCAGAUGA | 7964 |
| AD-1706291.1 | csascagcuaUfCfCfaucagaugauL96 | 6594 | asdTscadTcdTgaugdGaUfagcugugscsu | 7254 | AGCACAGCUAUCCAUCAGAUGAU | 7965 |
| AD-1706292.1 | ascsagcuauCfCfAfucagaugauuL96 | 6595 | asdAsucdAudCugaudGgAfuagcugusgsc | 7255 | GCACAGCUAUCCAUCAGAUGAUC | 7966 |
| AD-1706293.1 | csasgcuaucCfAfUfcagaugaucuL96 | 6596 | asdGsaudCadTcugadTgGfauagcugsusg | 7256 | CACAGCUAUCCAUCAGAUGAUCU | 7967 |
| AD-1706294.1 | asgscuauccAfUfCfagaugaucuuL96 | 6597 | asdAsgadTcdAucugdAuGfgauagcusgsu | 7257 | ACAGCUAUCCAUCAGAUGAUCUA | 7968 |
| AD-1706295.1 | gscsuauccaUfCfAfgaugaucuauL96 | 6598 | asdTsagdAudCaucudGaUfggauagcsusg | 7258 | CAGCUAUCCAUCAGAUGAUCUAC | 7969 |
| AD-1706296.1 | csusauccauCfAfGfaugaucuacuL96 | 6599 | asdGsuadGadTcaucdTgAfuggauagscsu | 7259 | AGCUAUCCAUCAGAUGAUCUACU | 7970 |
| AD-1706297.1 | usasuccaucAfGfAfugaucuacuuL96 | 6600 | asdAsgudAgdAucaudCuGfauggauasgsc | 7260 | GCUAUCCAUCAGAUGAUCUACUU | 7971 |
| AD-1706298.1 | asusccaucaGfAfUfgaucuacuuuL96 | 6601 | asdAsagdTadGaucadTcUfgauggausasg | 7261 | CUAUCCAUCAGAUGAUCUACUUU | 7972 |
| AD-1706299.1 | uscscaucagAfUfGfaucuacuuuuL96 | 6602 | asdAsaadGudAgaucdAuCfugauggasusa | 7262 | UAUCCAUCAGAUGAUCUACUUUC | 7973 |
| AD-1706300.1 | cscsaucagaUfGfAfucuacuuucuL96 | 6603 | asdGsaadAgdTagaudCaUfcugauggsasu | 7263 | AUCCAUCAGAUGAUCUACUUUCA | 7974 |
| AD-1706301.1 | csasucagauGfAfUfcuacuuucauL96 | 6604 | asdTsgadAadGuagadTcAfucugaugsgsa | 7264 | UCCAUCAGAUGAUCUACUUUCAG | 7975 |
| AD-1706302.1 | asuscagaugAfUfCfuacuuucaguL96 | 6605 | asdCsugdAadAguagdAuCfaucugausgsg | 7265 | CCAUCAGAUGAUCUACUUUCAGC | 7976 |
| AD-1706303.1 | uscsagaugaUfCfUfacuuucagcuL96 | 6606 | asdGscudGadAaguadGaUfcaucugasusg | 7266 | CAUCAGAUGAUCUACUUUCAGCC | 7977 |
| AD-1706304.1 | csasgaugauCfUfAfcuuucagccuL96 | 6607 | asdGsgcdTgdAaagudAgAfucaucugsasu | 7267 | AUCAGAUGAUCUACUUUCAGCCU | 7978 |
| AD-1706305.1 | asgsaugaucUfAfCfuuucagccuuL96 | 6608 | asdAsggdCudGaaagdTaGfaucaucusgsa | 7268 | UCAGAUGAUCUACUUUCAGCCUU | 7979 |
| AD-1706306.1 | gsasugaucuAfCfUfuucagccuuuL96 | 6609 | asdAsagdGcdTgaaadGuAfgaucaucsusg | 7269 | CAGAUGAUCUACUUUCAGCCUUC | 7980 |
| AD-1706307.1 | asusgaucuaCfUfUfucagccuucuL96 | 6610 | asdGsaadGgdCugaadAgUfagaucauscsu | 7270 | AGAUGAUCUACUUUCAGCCUUCC | 7981 |
| AD-1706308.1 | usgsaucuacUfUfUfcagccuuccuL96 | 6611 | asdGsgadAgdGcugadAaGfuagaucasusc | 7271 | GAUGAUCUACUUUCAGCCUUCCU | 7982 |
| AD-1706309.1 | gsasucuacuUfUfCfagccuuccuuL96 | 6612 | asdAsggdAadGgcugdAaAfguagaucsasu | 7272 | AUGAUCUACUUUCAGCCUUCCUG | 7983 |
| AD-1706310.1 | asuscuacuuUfCfAfgccuuccuguL96 | 6613 | asdCsagdGadAggcudGaAfaguagauscsa | 7273 | UGAUCUACUUUCAGCCUUCCUGA | 7984 |
| AD-1706311.1 | uscsuacuuuCfAfGfccuuccugauL96 | 6614 | asdTscadGgdAaggcdTgAfaaguagasusc | 7274 | GAUCUACUUUCAGCCUUCCUGAG | 7985 |
| AD-1706312.1 | csusacuuucAfGfCfcuuccugaguL96 | 6615 | asdCsucdAgdGaaggdCuGfaaaguagsasu | 7275 | AUCUACUUUCAGCCUUCCUGAGU | 7986 |
| AD-1706313.1 | usascuuucaGfCfCfuuccugaguuL96 | 6616 | asdAscudCadGgaagdGcUfgaaaguasgsa | 7276 | UCUACUUUCAGCCUUCCUGAGUC | 7987 |
| AD-1706314.1 | ascsuuucagCfCfUfuccugagucuL96 | 6617 | asdGsacdTcdAggaadGgCfugaaagusasg | 7277 | CUACUUUCAGCCUUCCUGAGUCC | 7988 |
| AD-1706318.1 | uscsagccuuCfCfUfgagucccaguL96 | 6618 | asdCsugdGgdAcucadGgAfaggcugasasa | 7278 | UUUCAGCCUUCCUGAGUCCCAGA | 7989 |
| AD-1706337.1 | gsascaauagAfAfGfacagguggcuL96 | 6619 | asdGsccdAcdCugucdTuCfuauugucsusg | 7279 | CAGACAAUAGAAGACAGGUGGCU | 7990 |
| AD-1706338.1 | ascsaauagaAfGfAfcagguggcuuL96 | 6620 | asdAsgcdCadCcugudCuUfcuauuguscsu | 7280 | AGACAAUAGAAGACAGGUGGCUG | 7991 |
| AD-1706380.1 | gsusggcaguGfGfUfgucugcuguuL96 | 6621 | asdAscadGcdAgacadCcAfcugccacsasc | 7281 | GUGUGGCAGUGGUGUCUGCUGUC | 7992 |
| AD-1706381.1 | usgsgcagugGfUfGfucugcugucuL96 | 6622 | asdGsacdAgdCagacdAcCfacugccascsa | 7282 | UGUGGCAGUGGUGUCUGCUGUCA | 7993 |
| AD-1706382.1 | gsgscaguggUfGfUfcugcugucauL96 | 6623 | asdTsgadCadGcagadCaCfcacugccsasc | 7283 | GUGGCAGUGGUGUCUGCUGUCAC | 7994 |
| AD-1706383.1 | gscsagugguGfUfCfugcugucacuL96 | 6624 | asdGsugdAcdAgcagdAcAfccacugcscsa | 7284 | UGGCAGUGGUGUCUGCUGUCACU | 7995 |
| AD-1706384.1 | csasguggugUfCfUfgcugucacuuL96 | 6625 | asdAsgudGadCagcadGaCfaccacugscsc | 7285 | GGCAGUGGUGUCUGCUGUCACUG | 7996 |
| AD-1706385.1 | asgsugguguCfUfGfcugucacuguL96 | 6626 | asdCsagdTgdAcagcdAgAfcaccacusgsc | 7286 | GCAGUGGUGUCUGCUGUCACUGU | 7997 |
| AD-1706386.1 | gsusggugucUfGfCfugucacuguuL96 | 6627 | asdAscadGudGacagdCaGfacaccacsusg | 7287 | CAGUGGUGUCUGCUGUCACUGUG | 7998 |
| AD-1706387.1 | usgsgugucuGfCfUfgucacuguguL96 | 6628 | asdCsacdAgdTgacadGcAfgacaccascsu | 7288 | AGUGGUGUCUGCUGUCACUGUGC | 7999 |
| AD-1706388.1 | gsgsugucugCfUfGfucacugugcuL96 | 6629 | asdGscadCadGugacdAgCfagacaccsasc | 7289 | GUGGUGUCUGCUGUCACUGUGCC | 8000 |
| AD-1706407.1 | csasgacucaAfCfAfgacggagcauL96 | 6630 | asdTsgcdTcdCgucudGuUfgagucugsasu | 7290 | AUCAGACUCAACAGACGGAGCAA | 8001 |
| AD-1706408.1 | asgsacucaaCfAfGfacggagcaauL96 | 6631 | asdTsugdCudCcgucdTgUfugagucusgsa | 7291 | UCAGACUCAACAGACGGAGCAAC | 8002 |
| AD-1706409.1 | gsascucaacAfGfAfcggagcaacuL96 | 6632 | asdGsuudGcdTccgudCuGfuugagucsusg | 7292 | CAGACUCAACAGACGGAGCAACU | 8003 |
| AD-1706410.1 | ascsucaacaGfAfCfggagcaacuuL96 | 6633 | asdAsgudTgdCuccgdTcUfguugaguscsu | 7293 | AGACUCAACAGACGGAGCAACUG | 8004 |
| AD-1706411.1 | csuscaacagAfCfGfgagcaacuguL96 | 6634 | asdCsagdTudGcuccdGuCfuguugagsusc | 7294 | GACUCAACAGACGGAGCAACUGC | 8005 |
| AD-1706412.1 | uscsaacagaCfGfGfagcaacugcuL96 | 6635 | asdGscadGudTgcucdCgUfcuguugasgsu | 7295 | ACUCAACAGACGGAGCAACUGCC | 8006 |
| AD-1706413.1 | csasacagacGfGfAfgcaacugccuL96 | 6636 | asdGsgcdAgdTugcudCcGfucuguugsasg | 7296 | CUCAACAGACGGAGCAACUGCCA | 8007 |
| AD-1706414.1 | asascagacgGfAfGfcaacugccauL96 | 6637 | asdTsggdCadGuugcdTcCfgucuguusgsa | 7297 | UCAACAGACGGAGCAACUGCCAU | 8008 |
| AD-1706415.1 | ascsagacggAfGfCfaacugccauuL96 | 6638 | asdAsugdGcdAguugdCuCfcgucugususg | 7298 | CAACAGACGGAGCAACUGCCAUC | 8009 |
| AD-1706416.1 | csasgacggaGfCfAfacugccaucuL96 | 6639 | asdGsaudGgdCaguudGcUfccgucugsusu | 7299 | AACAGACGGAGCAACUGCCAUCC | 8010 |
| AD-1706417.1 | asgsacggagCfAfAfcugccauccuL96 | 6640 | asdGsgadTgdGcagudTgCfuccgucusgsu | 7300 | ACAGACGGAGCAACUGCCAUCCG | 8011 |
| AD-1706418.1 | gsascggagcAfAfCfugccauccguL96 | 6641 | asdCsggdAudGgcagdTuGfcuccgucsusg | 7301 | CAGACGGAGCAACUGCCAUCCGA | 8012 |
| AD-1706419.1 | ascsggagcaAfCfUfgccauccgauL96 | 6642 | asdTscgdGadTggcadGuUfgcuccguscsu | 7302 | AGACGGAGCAACUGCCAUCCGAG | 8013 |
| AD-1706420.1 | csgsgagcaaCfUfGfccauccgaguL96 | 6643 | asdCsucdGgdAuggcdAgUfugcuccgsusc | 7303 | GACGGAGCAACUGCCAUCCGAGG | 8014 |
| AD-1706421.1 | gsgsagcaacUfGfCfcauccgagguL96 | 6644 | asdCscudCgdGauggdCaGfuugcuccsgsu | 7304 | ACGGAGCAACUGCCAUCCGAGGC | 8015 |
| AD-1706423.1 | asgscaacugCfCfAfuccgaggcuuL96 | 6645 | asdAsgcdCudCggaudGgCfaguugcuscsc | 7305 | GGAGCAACUGCCAUCCGAGGCUC | 8016 |
| AD-1706424.1 | gscsaacugcCfAfUfccgaggcucuL96 | 6646 | asdGsagdCcdTcggadTgGfcaguugcsusc | 7306 | GAGCAACUGCCAUCCGAGGCUCC | 8017 |
| AD-1706425.1 | csasacugccAfUfCfcgaggcuccuL96 | 6647 | asdGsgadGcdCucggdAuGfgcaguugscsu | 7307 | AGCAACUGCCAUCCGAGGCUCCU | 8018 |
| AD-1706520.1 | csasagcagaAfCfGfagcucugguuL96 | 6648 | asdAsccdAgdAgcucdGuUfcugcuugsgsg | 7308 | CCCAAGCAGAACGAGCUCUGGUG | 8019 |
| AD-1706521.1 | asasgcagaaCfGfAfgcucugguguL96 | 6649 | asdCsacdCadGagcudCgUfucugcuusgsg | 7309 | CCAAGCAGAACGAGCUCUGGUGC | 8020 |
| AD-1706522.1 | asgscagaacGfAfGfcucuggugcuL96 | 6650 | asdGscadCcdAgagcdTcGfuucugcususg | 7310 | CAAGCAGAACGAGCUCUGGUGCU | 8021 |
| AD-1706523.1 | gscsagaacgAfGfCfucuggugcuuL96 | 6651 | asdAsgcdAcdCagagdCuCfguucugcsusu | 7311 | AAGCAGAACGAGCUCUGGUGCUG | 8022 |
| AD-1706524.1 | csasgaacgaGfCfUfcuggugcuguL96 | 6652 | asdCsagdCadCcagadGcUfcguucugscsu | 7312 | AGCAGAACGAGCUCUGGUGCUGG | 8023 |
| AD-1706525.1 | asgsaacgagCfUfCfuggugcugguL96 | 6653 | asdCscadGcdAccagdAgCfucguucusgsc | 7313 | GCAGAACGAGCUCUGGUGCUGGA | 8024 |
| AD-1706526.1 | gsasacgagcUfCfUfggugcuggauL96 | 6654 | asdTsccdAgdCaccadGaGfcucguucsusg | 7314 | CAGAACGAGCUCUGGUGCUGGAG | 8025 |
| AD-1706527.1 | asascgagcuCfUfGfgugcuggaguL96 | 6655 | asdCsucdCadGcaccdAgAfgcucguuscsu | 7315 | AGAACGAGCUCUGGUGCUGGAGC | 8026 |
| AD-1706528.1 | ascsgagcucUfGfGfugcuggagcuL96 | 6656 | asdGscudCcdAgcacdCaGfagcucgususc | 7316 | GAACGAGCUCUGGUGCUGGAGCU | 8027 |
| AD-1706530.1 | gsasgcucugGfUfGfcuggagcuauL96 | 6657 | asdTsagdCudCcagcdAcCfagagcucsgsu | 7317 | ACGAGCUCUGGUGCUGGAGCUAG | 8028 |
| AD-1706532.1 | gscsucugguGfCfUfggagcuagcuL96 | 6658 | asdGscudAgdCuccadGcAfccagagcsusc | 7318 | GAGCUCUGGUGCUGGAGCUAGCC | 8029 |
| AD-1706533.1 | csuscuggugCfUfGfgagcuagccuL96 | 6659 | asdGsgcdTadGcuccdAgCfaccagagscsu | 7319 | AGCUCUGGUGCUGGAGCUAGCCA | 8030 |
| AD-1706534.1 | uscsuggugcUfGfGfagcuagccauL96 | 6660 | asdTsggdCudAgcucdCaGfcaccagasgsc | 7320 | GCUCUGGUGCUGGAGCUAGCCAA | 8031 |
| AD-1706535.1 | csusggugcuGfGfAfgcuagccaauL96 | 6661 | asdTsugdGcdTagcudCcAfgcaccagsasg | 7321 | CUCUGGUGCUGGAGCUAGCCAAG | 8032 |
| AD-1706536.1 | usgsgugcugGfAfGfcuagccaaguL96 | 6662 | asdCsuudGgdCuagcdTcCfagcaccasgsa | 7322 | UCUGGUGCUGGAGCUAGCCAAGC | 8033 |
| AD-1706537.1 | gsgsugcuggAfGfCfuagccaagcuL96 | 6663 | asdGscudTgdGcuagdCuCfcagcaccsasg | 7323 | CUGGUGCUGGAGCUAGCCAAGCA | 8034 |
| AD-1706538.1 | gsusgcuggaGfCfUfagccaagcauL96 | 6664 | asdTsgcdTudGgcuadGcUfccagcacscsa | 7324 | UGGUGCUGGAGCUAGCCAAGCAG | 8035 |
| AD-1706539.1 | usgscuggagCfUfAfgccaagcaguL96 | 6665 | asdCsugdCudTggcudAgCfuccagcascsc | 7325 | GGUGCUGGAGCUAGCCAAGCAGC | 8036 |
| AD-1706543.1 | gsgsagcuagCfCfAfagcagcaaauL96 | 6666 | asdTsuudGcdTgcuudGgCfuagcuccsasg | 7326 | CUGGAGCUAGCCAAGCAGCAAAU | 8037 |
| AD-1706544.1 | gsasgcuagcCfAfAfgcagcaaauuL96 | 6667 | asdAsuudTgdCugcudTgGfcuagcucscsa | 7327 | UGGAGCUAGCCAAGCAGCAAAUC | 8038 |
| AD-1706545.1 | asgscuagccAfAfGfcagcaaaucuL96 | 6668 | asdGsaudTudGcugcdTuGfgcuagcuscsc | 7328 | GGAGCUAGCCAAGCAGCAAAUCC | 8039 |
| AD-1706546.1 | gscsuagccaAfGfCfagcaaauccuL96 | 6669 | asdGsgadTudTgcugdCuUfggcuagcsusc | 7329 | GAGCUAGCCAAGCAGCAAAUCCU | 8040 |
| AD-1706547.1 | csusagccaaGfCfAfgcaaauccuuL96 | 6670 | asdAsggdAudTugcudGcUfuggcuagscsu | 7330 | AGCUAGCCAAGCAGCAAAUCCUG | 8041 |
| AD-1706548.1 | usasgccaagCfAfGfcaaauccuguL96 | 6671 | asdCsagdGadTuugcdTgCfuuggcuasgsc | 7331 | GCUAGCCAAGCAGCAAAUCCUGG | 8042 |
| AD-1706549.1 | asgsccaagcAfGfCfaaauccugguL96 | 6672 | asdCscadGgdAuuugdCuGfcuuggcusasg | 7332 | CUAGCCAAGCAGCAAAUCCUGGA | 8043 |
| AD-1706550.1 | gscscaagcaGfCfAfaauccuggauL96 | 6673 | asdTsccdAgdGauuudGcUfgcuuggcsusa | 7333 | UAGCCAAGCAGCAAAUCCUGGAU | 8044 |
| AD-1706578.1 | ascscugaccAfGfUfcgucccagauL96 | 6674 | asdTscudGgdGacgadCuGfgucaggusgsc | 7334 | GCACCUGACCAGUCGUCCCAGAA | 8045 |
| AD-1706579.1 | cscsugaccaGfUfCfgucccagaauL96 | 6675 | asdTsucdTgdGgacgdAcUfggucaggsusg | 7335 | CACCUGACCAGUCGUCCCAGAAU | 8046 |
| AD-1706580.1 | csusgaccagUfCfGfucccagaauuL96 | 6676 | asdAsuudCudGggacdGaCfuggucagsgsu | 7336 | ACCUGACCAGUCGUCCCAGAAUA | 8047 |
| AD-1706581.1 | usgsaccaguCfGfUfcccagaauauL96 | 6677 | asdTsaudTcdTgggadCgAfcuggucasgsg | 7337 | CCUGACCAGUCGUCCCAGAAUAA | 8048 |
| AD-1706582.1 | gsasccagucGfUfCfccagaauaauL96 | 6678 | asdTsuadTudCugggdAcGfacuggucsasg | 7338 | CUGACCAGUCGUCCCAGAAUAAC | 8049 |
| AD-1706583.1 | ascscagucgUfCfCfcagaauaacuL96 | 6679 | asdGsuudAudTcuggdGaCfgacugguscsa | 75 | UGACCAGUCGUCCCAGAAUAACU | 8050 |
| AD-1706584.1 | cscsagucguCfCfCfagaauaacuuL96 | 6680 | asdAsgudTadTucugdGgAfcgacuggsusc | 7339 | GACCAGUCGUCCCAGAAUAACUC | 8051 |
| AD-1706585.1 | csasgucgucCfCfAfgaauaacucuL96 | 6681 | asdGsagdTudAuucudGgGfacgacugsgsu | 7340 | ACCAGUCGUCCCAGAAUAACUCA | 8052 |
| AD-1706586.1 | asgsucguccCfAfGfaauaacucauL96 | 6682 | asdTsgadGudTauucdTgGfgacgacusgsg | 7341 | CCAGUCGUCCCAGAAUAACUCAU | 8053 |
| AD-1706587.1 | gsuscgucccAfGfAfauaacucauuL96 | 6683 | asdAsugdAgdTuauudCuGfggacgacsusg | 7342 | CAGUCGUCCCAGAAUAACUCAUC | 8054 |
| AD-1706588.1 | uscsgucccaGfAfAfuaacucaucuL96 | 6684 | asdGsaudGadGuuaudTcUfgggacgascsu | 7343 | AGUCGUCCCAGAAUAACUCAUCC | 8055 |
| AD-1706589.1 | csgsucccagAfAfUfaacucauccuL96 | 6685 | asdGsgadTgdAguuadTuCfugggacgsasc | 7344 | GUCGUCCCAGAAUAACUCAUCCU | 8056 |
| AD-1706590.1 | gsuscccagaAfUfAfacucauccuuL96 | 6686 | asdAsggdAudGaguudAuUfcugggacsgsa | 7345 | UCGUCCCAGAAUAACUCAUCCUC | 8057 |
| AD-1706591.1 | uscsccagaaUfAfAfcucauccucuL96 | 6687 | asdGsagdGadTgagudTaUfucugggascsg | 7346 | CGUCCCAGAAUAACUCAUCCUCC | 8058 |
| AD-1706592.1 | cscscagaauAfAfCfucauccuccuL96 | 6688 | asdGsgadGgdAugagdTuAfuucugggsasc | 7347 | GUCCCAGAAUAACUCAUCCUCCA | 8059 |
| AD-1706593.1 | cscsagaauaAfCfUfcauccuccauL96 | 6689 | asdTsggdAgdGaugadGuUfauucuggsgsa | 78 | UCCCAGAAUAACUCAUCCUCCAC | 8060 |
| AD-1706594.1 | csasgaauaaCfUfCfauccuccacuL96 | 6690 | asdGsugdGadGgaugdAgUfuauucugsgsg | 7348 | CCCAGAAUAACUCAUCCUCCACC | 8061 |
| AD-1706635.1 | gsgsgaguguGfGfCfuccagggaauL96 | 6691 | asdTsucdCcdTggagdCcAfcacucccsusg | 7349 | CAGGGAGUGUGGCUCCAGGGAAU | 8062 |
| AD-1706636.1 | gsgsagugugGfCfUfccagggaauuL96 | 6692 | asdAsuudCcdCuggadGcCfacacuccscsu | 7350 | AGGGAGUGUGGCUCCAGGGAAUG | 8063 |
| AD-1706637.1 | gsasguguggCfUfCfcagggaauguL96 | 6693 | asdCsaudTcdCcuggdAgCfcacacucscsc | 7351 | GGGAGUGUGGCUCCAGGGAAUGG | 8064 |
| AD-1706638.1 | asgsuguggcUfCfCfagggaaugguL96 | 6694 | asdCscadTudCccugdGaGfccacacuscsc | 7352 | GGAGUGUGGCUCCAGGGAAUGGG | 8065 |
| AD-1706639.1 | gsasggagguCfAfUfcagcuuugcuL96 | 6695 | asdGscadAadGcugadTgAfccuccucscsc | 7353 | GGGAGGAGGUCAUCAGCUUUGCU | 8066 |
| AD-1706640.1 | asgsgaggucAfUfCfagcuuugcuuL96 | 6696 | asdAsgcdAadAgcugdAuGfaccuccuscsc | 7354 | GGAGGAGGUCAUCAGCUUUGCUA | 8067 |
| AD-1706641.1 | gsgsaggucaUfCfAfgcuuugcuauL96 | 6697 | asdTsagdCadAagcudGaUfgaccuccsusc | 7355 | GAGGAGGUCAUCAGCUUUGCUAC | 8068 |
| AD-1706642.1 | gsasggucauCfAfGfcuuugcuacuL96 | 6698 | asdGsuadGcdAaagcdTgAfugaccucscsu | 7356 | AGGAGGUCAUCAGCUUUGCUACU | 8069 |
| AD-1706643.1 | asgsgucaucAfGfCfuuugcuacuuL96 | 6699 | asdAsgudAgdCaaagdCuGfaugaccuscsc | 7357 | GGAGGUCAUCAGCUUUGCUACUG | 8070 |
| AD-1706644.1 | gsgsucaucaGfCfUfuugcuacuguL96 | 6700 | asdCsagdTadGcaaadGcUfgaugaccsusc | 7358 | GAGGUCAUCAGCUUUGCUACUGU | 8071 |
| AD-1706645.1 | gsuscaucagCfUfUfugcuacuguuL96 | 6701 | asdAscadGudAgcaadAgCfugaugacscsu | 7359 | AGGUCAUCAGCUUUGCUACUGUC | 8072 |
| AD-1706646.1 | uscsaucagcUfUfUfgcuacugucuL96 | 6702 | asdGsacdAgdTagcadAaGfcugaugascsc | 7360 | GGUCAUCAGCUUUGCUACUGUCA | 8073 |
| AD-1706649.1 | uscsagcuuuGfCfUfacugucacauL96 | 6703 | asdTsgudGadCaguadGcAfaagcugasusg | 7361 | CAUCAGCUUUGCUACUGUCACAG | 8074 |
| AD-1706650.1 | csasgcuuugCfUfAfcugucacaguL96 | 6704 | asdCsugdTgdAcagudAgCfaaagcugsasu | 7362 | AUCAGCUUUGCUACUGUCACAGA | 8075 |
| AD-1706651.1 | asgscuuugcUfAfCfugucacagauL96 | 6705 | asdTscudGudGacagdTaGfcaaagcusgsa | 7363 | UCAGCUUUGCUACUGUCACAGAC | 8076 |
| AD-1706652.1 | gscsuuugcuAfCfUfgucacagacuL96 | 6706 | asdGsucdTgdTgacadGuAfgcaaagcsusg | 7364 | CAGCUUUGCUACUGUCACAGACU | 8077 |
| AD-1706653.1 | csusuugcuaCfUfGfucacagacuuL96 | 6707 | asdAsgudCudGugacdAgUfagcaaagscsu | 7365 | AGCUUUGCUACUGUCACAGACUC | 8078 |
| AD-1706654.1 | ususugcuacUfGfUfcacagacucuL96 | 6708 | asdGsagdTcdTgugadCaGfuagcaaasgsc | 7366 | GCUUUGCUACUGUCACAGACUCC | 8079 |
| AD-1706655.1 | ususgcuacuGfUfCfacagacuccuL96 | 6709 | asdGsgadGudCugugdAcAfguagcaasasg | 7367 | CUUUGCUACUGUCACAGACUCCA | 8080 |
| AD-1706656.1 | usgscuacugUfCfAfcagacuccauL96 | 6710 | asdTsggdAgdTcugudGaCfaguagcasasa | 7368 | UUUGCUACUGUCACAGACUCCAC | 8081 |
| AD-1706660.1 | ascsugucacAfGfAfcuccacuucuL96 | 6711 | asdGsaadGudGgagudCuGfugacagusasg | 7369 | CUACUGUCACAGACUCCACUUCA | 8082 |
| AD-1706661.1 | csusgucacaGfAfCfuccacuucauL96 | 6712 | asdTsgadAgdTggagdTcUfgugacagsusa | 7370 | UACUGUCACAGACUCCACUUCAG | 8083 |
| AD-1706662.1 | usgsucacagAfCfUfccacuucaguL96 | 6713 | asdCsugdAadGuggadGuCfugugacasgsu | 82 | ACUGUCACAGACUCCACUUCAGC | 8084 |
| AD-1706664.1 | uscsacagacUfCfCfacuucagccuL96 | 6714 | asdGsgcdTgdAagugdGaGfucugugascsa | 7371 | UGUCACAGACUCCACUUCAGCCU | 8085 |
| AD-1706665.1 | csascagacuCfCfAfcuucagccuuL96 | 6715 | asdAsggdCudGaagudGgAfgucugugsasc | 7372 | GUCACAGACUCCACUUCAGCCUA | 8086 |
| AD-1706666.1 | ascsagacucCfAfCfuucagccuauL96 | 6716 | asdTsagdGcdTgaagdTgGfagucugusgsa | 7373 | UCACAGACUCCACUUCAGCCUAC | 8087 |
| AD-1706667.1 | csasgacuccAfCfUfucagccuacuL96 | 6717 | asdGsuadGgdCugaadGuGfgagucugsusg | 7374 | CACAGACUCCACUUCAGCCUACA | 8088 |
| AD-1706668.1 | asgsacuccaCfUfUfcagccuacauL96 | 6718 | asdTsgudAgdGcugadAgUfggagucusgsu | 7375 | ACAGACUCCACUUCAGCCUACAG | 8089 |
| AD-1706669.1 | gsascuccacUfUfCfagccuacaguL96 | 6719 | asdCsugdTadGgcugdAaGfuggagucsusg | 7376 | CAGACUCCACUUCAGCCUACAGC | 8090 |
| AD-1706670.1 | ascsuccacuUfCfAfgccuacagcuL96 | 6720 | asdGscudGudAggcudGaAfguggaguscsu | 7377 | AGACUCCACUUCAGCCUACAGCU | 8091 |
| AD-1706671.1 | csusccacuuCfAfGfccuacagcuuL96 | 6721 | asdAsgcdTgdTaggcdTgAfaguggagsusc | 7378 | GACUCCACUUCAGCCUACAGCUC | 8092 |
| AD-1706672.1 | uscscacuucAfGfCfcuacagcucuL96 | 6722 | asdGsagdCudGuaggdCuGfaaguggasgsu | 7379 | ACUCCACUUCAGCCUACAGCUCC | 8093 |
| AD-1706673.1 | cscsacuucaGfCfCfuacagcuccuL96 | 6723 | asdGsgadGcdTguagdGcUfgaaguggsasg | 7380 | CUCCACUUCAGCCUACAGCUCCC | 8094 |
| AD-1706674.1 | csascuucagCfCfUfacagcucccuL96 | 6724 | asdGsggdAgdCuguadGgCfugaagugsgsa | 7381 | UCCACUUCAGCCUACAGCUCCCU | 8095 |
| AD-1706675.1 | ascsuucagcCfUfAfcagcucccuuL96 | 6725 | asdAsggdGadGcugudAgGfcugaagusgsg | 7382 | CCACUUCAGCCUACAGCUCCCUG | 8096 |
| AD-1706678.1 | uscsagccuaCfAfGfcucccugcuuL96 | 6726 | asdAsgcdAgdGgagcdTgUfaggcugasasg | 7383 | CUUCAGCCUACAGCUCCCUGCUC | 8097 |
| AD-1706729.1 | csasccuguaCfCfAfugcccgccuuL96 | 6727 | asdAsggdCgdGgcaudGgUfacaggugsgsu | 7384 | ACCACCUGUACCAUGCCCGCCUG | 8098 |
| AD-1706731.1 | cscsuguaccAfUfGfcccgccuguuL96 | 6728 | asdAscadGgdCgggcdAuGfguacaggsusg | 7385 | CACCUGUACCAUGCCCGCCUGUG | 8099 |
| AD-1706732.1 | csusguaccaUfGfCfccgccuguguL96 | 6729 | asdCsacdAgdGcgggdCaUfgguacagsgsu | 7386 | ACCUGUACCAUGCCCGCCUGUGG | 8100 |
| AD-1706735.1 | usasccaugcCfCfGfccuguggcuuL96 | 6730 | asdAsgcdCadCaggcdGgGfcaugguascsa | 7387 | UGUACCAUGCCCGCCUGUGGCUG | 8101 |
| AD-1706745.1 | csascccuucCfUfGfgcacucuuuuL96 | 6731 | asdAsaadGadGugccdAgGfaagggugsgsg | 7388 | CCCACCCUUCCUGGCACUCUUUG | 8102 |
| AD-1706746.1 | ascsccuuccUfGfGfcacucuuuguL96 | 6732 | asdCsaadAgdAgugcdCaGfgaagggusgsg | 7389 | CCACCCUUCCUGGCACUCUUUGC | 8103 |
| AD-1706747.1 | cscscuuccuGfGfCfacucuuugcuL96 | 6733 | asdGscadAadGagugdCcAfggaagggsusg | 7390 | CACCCUUCCUGGCACUCUUUGCU | 8104 |
| AD-1706748.1 | cscsuuccugGfCfAfcucuuugcuuL96 | 6734 | asdAsgcdAadAgagudGcCfaggaaggsgsu | 7391 | ACCCUUCCUGGCACUCUUUGCUU | 8105 |
| AD-1706749.1 | csusuccuggCfAfCfucuuugcuuuL96 | 6735 | asdAsagdCadAagagdTgCfcaggaagsgsg | 7392 | CCCUUCCUGGCACUCUUUGCUUG | 8106 |
| AD-1706750.1 | ususccuggcAfCfUfcuuugcuuguL96 | 6736 | asdCsaadGcdAaagadGuGfccaggaasgsg | 7393 | CCUUCCUGGCACUCUUUGCUUGA | 8107 |
| AD-1706751.1 | uscscuggcaCfUfCfuuugcuugauL96 | 6737 | asdTscadAgdCaaagdAgUfgccaggasasg | 7394 | CUUCCUGGCACUCUUUGCUUGAG | 8108 |
| AD-1706752.1 | cscsuggcacUfCfUfuugcuugaguL96 | 6738 | asdCsucdAadGcaaadGaGfugccaggsasa | 7395 | UUCCUGGCACUCUUUGCUUGAGG | 8109 |
| AD-1706753.1 | csusggcacuCfUfUfugcuugagguL96 | 6739 | asdCscudCadAgcaadAgAfgugccagsgsa | 7396 | UCCUGGCACUCUUUGCUUGAGGA | 8110 |
| AD-1706754.1 | usgsgcacucUfUfUfgcuugaggauL96 | 6740 | asdTsccdTcdAagcadAaGfagugccasgsg | 7397 | CCUGGCACUCUUUGCUUGAGGAU | 8111 |
| AD-1706755.1 | gsgscacucuUfUfGfcuugaggauuL96 | 6741 | asdAsucdCudCaagcdAaAfgagugccsasg | 7398 | CUGGCACUCUUUGCUUGAGGAUC | 8112 |
| AD-1706756.1 | gscsacucuuUfGfCfuugaggaucuL96 | 6742 | asdGsaudCcdTcaagdCaAfagagugcscsa | 7399 | UGGCACUCUUUGCUUGAGGAUCU | 8113 |
| AD-1706758.1 | ascsucuuugCfUfUfgaggaucuuuL96 | 6743 | asdAsagdAudCcucadAgCfaaagagusgsc | 7400 | GCACUCUUUGCUUGAGGAUCUUC | 8114 |
| AD-1706759.1 | csuscuuugcUfUfGfaggaucuucuL96 | 6744 | asdGsaadGadTccucdAaGfcaaagagsusg | 7401 | CACUCUUUGCUUGAGGAUCUUCC | 8115 |
| AD-1706760.1 | uscsuuugcuUfGfAfggaucuuccuL96 | 6745 | asdGsgadAgdAuccudCaAfgcaaagasgsu | 7402 | ACUCUUUGCUUGAGGAUCUUCCG | 8116 |
| AD-1706761.1 | csusuugcuuGfAfGfgaucuuccguL96 | 6746 | asdCsggdAadGauccdTcAfagcaaagsasg | 84 | CUCUUUGCUUGAGGAUCUUCCGA | 8117 |
| AD-1706762.1 | ususugcuugAfGfGfaucuuccgauL96 | 6747 | asdTscgdGadAgaucdCuCfaagcaaasgsa | 7403 | UCUUUGCUUGAGGAUCUUCCGAU | 8118 |
| AD-1706763.1 | ususgcuugaGfGfAfucuuccgauuL96 | 6748 | asdAsucdGgdAagaudCcUfcaagcaasasg | 7404 | CUUUGCUUGAGGAUCUUCCGAUG | 8119 |
| AD-1706764.1 | usgscuugagGfAfUfcuuccgauguL96 | 6749 | asdCsaudCgdGaagadTcCfucaagcasasa | 7405 | UUUGCUUGAGGAUCUUCCGAUGG | 8120 |
| AD-1706765.1 | gscsuugaggAfUfCfuuccgaugguL96 | 6750 | asdCscadTcdGgaagdAuCfcucaagcsasa | 7406 | UUGCUUGAGGAUCUUCCGAUGGG | 8121 |
| AD-1706796.1 | csusggcugaGfCfAfccacaucacuL96 | 6751 | asdGsugdAudGuggudGcUfcagccagsgsa | 7407 | UCCUGGCUGAGCACCACAUCACC | 8122 |
| AD-1706797.1 | usgsgcugagCfAfCfcacaucaccuL96 | 6752 | asdGsgudGadTguggdTgCfucagccasgsg | 7408 | CCUGGCUGAGCACCACAUCACCA | 8123 |
| AD-1706798.1 | gsgscugagcAfCfCfacaucaccauL96 | 6753 | asdTsggdTgdAugugdGuGfcucagccsasg | 7409 | CUGGCUGAGCACCACAUCACCAA | 8124 |
| AD-1706799.1 | gscsugagcaCfCfAfcaucaccaauL96 | 6754 | asdTsugdGudGaugudGgUfgcucagcscsa | 7410 | UGGCUGAGCACCACAUCACCAAC | 8125 |
| AD-1706800.1 | csusgagcacCfAfCfaucaccaacuL96 | 6755 | asdGsuudGgdTgaugdTgGfugcucagscsc | 7411 | GGCUGAGCACCACAUCACCAACC | 8126 |
| AD-1706801.1 | usgsagcaccAfCfAfucaccaaccuL96 | 6756 | asdGsgudTgdGugaudGuGfgugcucasgsc | 7412 | GCUGAGCACCACAUCACCAACCU | 8127 |
| AD-1706802.1 | gsasgcaccaCfAfUfcaccaaccuuL96 | 6757 | asdAsggdTudGgugadTgUfggugcucsasg | 7413 | CUGAGCACCACAUCACCAACCUG | 8128 |
| AD-1706803.1 | asgscaccacAfUfCfaccaaccuguL96 | 6758 | asdCsagdGudTggugdAuGfuggugcuscsa | 7414 | UGAGCACCACAUCACCAACCUGG | 8129 |
| AD-1706804.1 | gscsaccacaUfCfAfccaaccugguL96 | 6759 | asdCscadGgdTuggudGaUfguggugcsusc | 7415 | GAGCACCACAUCACCAACCUGGG | 8130 |
| AD-1706807.1 | cscsacaucaCfCfAfaccugggcuuL96 | 6760 | asdAsgcdCcdAgguudGgUfgauguggsusg | 7416 | CACCACAUCACCAACCUGGGCUG | 8131 |
| AD-1706808.1 | csascaucacCfAfAfccugggcuguL96 | 6761 | asdCsagdCcdCaggudTgGfugaugugsgsu | 7417 | ACCACAUCACCAACCUGGGCUGG | 8132 |
| AD-1706809.1 | ascsaucaccAfAfCfcugggcugguL96 | 6762 | asdCscadGcdCcaggdTuGfgugaugusgsg | 7418 | CCACAUCACCAACCUGGGCUGGC | 8133 |
| AD-1706811.1 | asuscaccaaCfCfUfgggcuggcauL96 | 6763 | asdTsgcdCadGcccadGgUfuggugausgsu | 7419 | ACAUCACCAACCUGGGCUGGCAU | 8134 |
| AD-1706812.1 | uscsaccaacCfUfGfggcuggcauuL96 | 6764 | asdAsugdCcdAgcccdAgGfuuggugasusg | 7420 | CAUCACCAACCUGGGCUGGCAUA | 8135 |
| AD-1706831.1 | usasccuuaaCfUfCfugcccucuauL96 | 6765 | asdTsagdAgdGgcagdAgUfuaagguasusg | 7421 | CAUACCUUAACUCUGCCCUCUAG | 8136 |
| AD-1706832.1 | ascscuuaacUfCfUfgcccucuaguL96 | 6766 | asdCsuadGadGggcadGaGfuuaaggusasu | 7422 | AUACCUUAACUCUGCCCUCUAGU | 8137 |
| AD-1706833.1 | cscsuuaacuCfUfGfcccucuaguuL96 | 6767 | asdAscudAgdAgggcdAgAfguuaaggsusa | 7423 | UACCUUAACUCUGCCCUCUAGUG | 8138 |
| AD-1706834.1 | csusuaacucUfGfCfccucuaguguL96 | 6768 | asdCsacdTadGagggdCaGfaguuaagsgsu | 7424 | ACCUUAACUCUGCCCUCUAGUGG | 8139 |
| AD-1706835.1 | ususaacucuGfCfCfcucuagugguL96 | 6769 | asdCscadCudAgaggdGcAfgaguuaasgsg | 7425 | CCUUAACUCUGCCCUCUAGUGGC | 8140 |
| AD-1706836.1 | usasacucugCfCfCfucuaguggcuL96 | 6770 | asdGsccdAcdTagagdGgCfagaguuasasg | 7426 | CUUAACUCUGCCCUCUAGUGGCU | 8141 |
| AD-1706848.1 | asasgucuggUfGfUfccugaaacuuL96 | 6771 | asdAsgudTudCaggadCaCfcagacuuscsu | 7427 | AGAAGUCUGGUGUCCUGAAACUG | 8142 |
| AD-1706849.1 | asgsucugguGfUfCfcugaaacuguL96 | 6772 | asdCsagdTudTcaggdAcAfccagacususc | 7428 | GAAGUCUGGUGUCCUGAAACUGC | 8143 |
| AD-1706850.1 | gsuscuggugUfCfCfugaaacugcuL96 | 6773 | asdGscadGudTucagdGaCfaccagacsusu | 7429 | AAGUCUGGUGUCCUGAAACUGCA | 8144 |
| AD-1706851.1 | uscsugguguCfCfUfgaaacugcauL96 | 6774 | asdTsgcdAgdTuucadGgAfcaccagascsu | 7430 | AGUCUGGUGUCCUGAAACUGCAA | 8145 |
| AD-1706852.1 | csusggugucCfUfGfaaacugcaauL96 | 6775 | asdTsugdCadGuuucdAgGfacaccagsasc | 7431 | GUCUGGUGUCCUGAAACUGCAAC | 8146 |
| AD-1706853.1 | usgsguguccUfGfAfaacugcaacuL96 | 6776 | asdGsuudGcdAguuudCaGfgacaccasgsa | 7432 | UCUGGUGUCCUGAAACUGCAACU | 8147 |
| AD-1706854.1 | gsgsuguccuGfAfAfacugcaacuuL96 | 6777 | asdAsgudTgdCaguudTcAfggacaccsasg | 7433 | CUGGUGUCCUGAAACUGCAACUA | 8148 |
| AD-1706855.1 | gsusguccugAfAfAfcugcaacuauL96 | 6778 | asdTsagdTudGcagudTuCfaggacacscsa | 7434 | UGGUGUCCUGAAACUGCAACUAG | 8149 |
| AD-1706856.1 | usgsuccugaAfAfCfugcaacuaguL96 | 6779 | asdCsuadGudTgcagdTuUfcaggacascsc | 7435 | GGUGUCCUGAAACUGCAACUAGA | 8150 |
| AD-1706857.1 | gsusccugaaAfCfUfgcaacuagauL96 | 6780 | asdTscudAgdTugcadGuUfucaggacsasc | 7436 | GUGUCCUGAAACUGCAACUAGAC | 8151 |
| AD-1706858.1 | uscscugaaaCfUfGfcaacuagacuL96 | 6781 | asdGsucdTadGuugcdAgUfuucaggascsa | 7437 | UGUCCUGAAACUGCAACUAGACU | 8152 |
| AD-1706859.1 | cscsugaaacUfGfCfaacuagacuuL96 | 6782 | asdAsgudCudAguugdCaGfuuucaggsasc | 7438 | GUCCUGAAACUGCAACUAGACUG | 8153 |
| AD-1706860.1 | csusgaaacuGfCfAfacuagacuguL96 | 6783 | asdCsagdTcdTaguudGcAfguuucagsgsa | 7439 | UCCUGAAACUGCAACUAGACUGC | 8154 |
| AD-1706861.1 | usgsaaacugCfAfAfcuagacugcuL96 | 6784 | asdGscadGudCuagudTgCfaguuucasgsg | 7440 | CCUGAAACUGCAACUAGACUGCA | 8155 |
| AD-1706862.1 | gsasaacugcAfAfCfuagacugcauL96 | 6785 | asdTsgcdAgdTcuagdTuGfcaguuucsasg | 7441 | CUGAAACUGCAACUAGACUGCAG | 8156 |
| AD-1706863.1 | asasacugcaAfCfUfagacugcaguL96 | 6786 | asdCsugdCadGucuadGuUfgcaguuuscsa | 7442 | UGAAACUGCAACUAGACUGCAGA | 8157 |
| AD-1706864.1 | asascugcaaCfUfAfgacugcagauL96 | 6787 | asdTscudGcdAgucudAgUfugcaguususc | 7443 | GAAACUGCAACUAGACUGCAGAC | 8158 |
| AD-1706866.1 | csusgcaacuAfGfAfcugcagaccuL96 | 6788 | asdGsgudCudGcagudCuAfguugcagsusu | 7444 | AACUGCAACUAGACUGCAGACCC | 8159 |
| AD-1706873.1 | gsgscaacagCfAfCfaguuacugguL96 | 6789 | asdCscadGudAacugdTgCfuguugccsusu | 7445 | AAGGCAACAGCACAGUUACUGGA | 8160 |
| AD-1706874.1 | gscsaacagcAfCfAfguuacuggauL96 | 6790 | asdTsccdAgdTaacudGuGfcuguugcscsu | 7446 | AGGCAACAGCACAGUUACUGGAC | 8161 |
| AD-1706875.1 | csasacagcaCfAfGfuuacuggacuL96 | 6791 | asdGsucdCadGuaacdTgUfgcuguugscsc | 7447 | GGCAACAGCACAGUUACUGGACA | 8162 |
| AD-1706876.1 | asascagcacAfGfUfuacuggacauL96 | 6792 | asdTsgudCcdAguaadCuGfugcuguusgsc | 7448 | GCAACAGCACAGUUACUGGACAA | 8163 |
| AD-1706877.1 | ascsagcacaGfUfUfacuggacaauL96 | 6793 | asdTsugdTcdCaguadAcUfgugcugususg | 7449 | CAACAGCACAGUUACUGGACAAC | 8164 |
| AD-1706878.1 | csasgcacagUfUfAfcuggacaacuL96 | 6794 | asdGsuudGudCcagudAaCfugugcugsusu | 7450 | AACAGCACAGUUACUGGACAACC | 8165 |
| AD-1706879.1 | asgscacaguUfAfCfuggacaaccuL96 | 6795 | asdGsgudTgdTccagdTaAfcugugcusgsu | 7451 | ACAGCACAGUUACUGGACAACCG | 8166 |
| AD-1706908.1 | csusuggacaCfAfGfcaggacaccuL96 | 6796 | asdGsgudGudCcugcdTgUfguccaagsasg | 7452 | CUCUUGGACACAGCAGGACACCA | 8167 |
| AD-1706909.1 | ususggacacAfGfCfaggacaccauL96 | 6797 | asdTsggdTgdTccugdCuGfuguccaasgsa | 7453 | UCUUGGACACAGCAGGACACCAG | 8168 |
| AD-1706910.1 | usgsgacacaGfCfAfggacaccaguL96 | 6798 | asdCsugdGudGuccudGcUfguguccasasg | 7454 | CUUGGACACAGCAGGACACCAGC | 8169 |
| AD-1706911.1 | gsgsacacagCfAfGfgacaccagcuL96 | 6799 | asdGscudGgdTguccdTgCfuguguccsasa | 7455 | UUGGACACAGCAGGACACCAGCA | 8170 |
| AD-1706912.1 | gsascacagcAfGfGfacaccagcauL96 | 6800 | asdTsgcdTgdGugucdCuGfcugugucscsa | 7456 | UGGACACAGCAGGACACCAGCAG | 8171 |
| AD-1706913.1 | ascsacagcaGfGfAfcaccagcaguL96 | 6801 | asdCsugdCudGgugudCcUfgcuguguscsc | 7457 | GGACACAGCAGGACACCAGCAGC | 8172 |
| AD-1706914.1 | csascagcagGfAfCfaccagcagcuL96 | 6802 | asdGscudGcdTggugdTcCfugcugugsusc | 7458 | GACACAGCAGGACACCAGCAGCC | 8173 |
| AD-1706918.1 | gscsaggacaCfCfAfgcagcccuuuL96 | 6803 | asdAsagdGgdCugcudGgUfguccugcsusg | 7459 | CAGCAGGACACCAGCAGCCCUUC | 8174 |
| AD-1706921.1 | gsgsacaccaGfCfAfgcccuuccuuL96 | 6804 | asdAsggdAadGggcudGcUfgguguccsusg | 7460 | CAGGACACCAGCAGCCCUUCCUA | 8175 |
| AD-1706922.1 | gsascaccagCfAfGfcccuuccuauL96 | 6805 | asdTsagdGadAgggcdTgCfuggugucscsu | 7461 | AGGACACCAGCAGCCCUUCCUAG | 8176 |
| AD-1706923.1 | ascsaccagcAfGfCfccuuccuaguL96 | 6806 | asdCsuadGgdAagggdCuGfcugguguscsc | 7462 | GGACACCAGCAGCCCUUCCUAGA | 8177 |
| AD-1706925.1 | ascscagcagCfCfCfuuccuagaguL96 | 6807 | asdCsucdTadGgaagdGgCfugcuggusgsu | 7463 | ACACCAGCAGCCCUUCCUAGAGC | 8178 |
| AD-1706926.1 | cscsagcagcCfCfUfuccuagagcuL96 | 6808 | asdGscudCudAggaadGgGfcugcuggsusg | 7464 | CACCAGCAGCCCUUCCUAGAGCU | 8179 |
| AD-1706927.1 | csasgcagccCfUfUfccuagagcuuL96 | 6809 | asdAsgcdTcdTaggadAgGfgcugcugsgsu | 7465 | ACCAGCAGCCCUUCCUAGAGCUU | 8180 |
| AD-1706929.1 | gscsagcccuUfCfCfuagagcuuauL96 | 6810 | asdTsaadGcdTcuagdGaAfgggcugcsusg | 7466 | CAGCAGCCCUUCCUAGAGCUUAA | 8181 |
| AD-1706930.1 | csasgcccuuCfCfUfagagcuuaauL96 | 6811 | asdTsuadAgdCucuadGgAfagggcugscsu | 7467 | AGCAGCCCUUCCUAGAGCUUAAG | 8182 |
| AD-1706931.1 | asgscccuucCfUfAfgagcuuaaguL96 | 6812 | asdCsuudAadGcucudAgGfaagggcusgsc | 7468 | GCAGCCCUUCCUAGAGCUUAAGA | 8183 |
| AD-1706932.1 | gscsccuuccUfAfGfagcuuaagauL96 | 6813 | asdTscudTadAgcucdTaGfgaagggcsusg | 7469 | CAGCCCUUCCUAGAGCUUAAGAU | 8184 |
| AD-1706933.1 | cscscuuccuAfGfAfgcuuaagauuL96 | 6814 | asdAsucdTudAagcudCuAfggaagggscsu | 7470 | AGCCCUUCCUAGAGCUUAAGAUC | 8185 |
| AD-1706934.1 | cscsuuccuaGfAfGfcuuaagaucuL96 | 6815 | asdGsaudCudTaagcdTcUfaggaaggsgsc | 7471 | GCCCUUCCUAGAGCUUAAGAUCC | 8186 |
| AD-1706982.1 | ususacguagAfCfUfuccaggaacuL96 | 6816 | asdGsuudCcdTggaadGuCfuacguaasusg | 7472 | CAUUACGUAGACUUCCAGGAACU | 8187 |
| AD-1706983.1 | usascguagaCfUfUfccaggaacuuL96 | 6817 | asdAsgudTcdCuggadAgUfcuacguasasu | 7473 | AUUACGUAGACUUCCAGGAACUG | 8188 |
| AD-1706984.1 | ascsguagacUfUfCfcaggaacuguL96 | 6818 | asdCsagdTudCcuggdAaGfucuacgusasa | 7474 | UUACGUAGACUUCCAGGAACUGG | 8189 |
| AD-1706985.1 | csgsuagacuUfCfCfaggaacugguL96 | 6819 | asdCscadGudTccugdGaAfgucuacgsusa | 7475 | UACGUAGACUUCCAGGAACUGGG | 8190 |
| AD-1706987.1 | usasgacuucCfAfGfgaacugggauL96 | 6820 | asdTsccdCadGuuccdTgGfaagucuascsg | 7476 | CGUAGACUUCCAGGAACUGGGAU | 8191 |
| AD-1706988.1 | asgsacuuccAfGfGfaacugggauuL96 | 6821 | asdAsucdCcdAguucdCuGfgaagucusasc | 7477 | GUAGACUUCCAGGAACUGGGAUG | 8192 |
| AD-1706989.1 | gsascuuccaGfGfAfacugggauguL96 | 6822 | asdCsaudCcdCaguudCcUfggaagucsusa | 7478 | UAGACUUCCAGGAACUGGGAUGG | 8193 |
| AD-1706990.1 | ascsuuccagGfAfAfcugggaugguL96 | 6823 | asdCscadTcdCcagudTcCfuggaaguscsu | 7479 | AGACUUCCAGGAACUGGGAUGGC | 8194 |
| AD-1706991.1 | csusuccaggAfAfCfugggauggcuL96 | 6824 | asdGsccdAudCccagdTuCfcuggaagsusc | 7480 | GACUUCCAGGAACUGGGAUGGCG | 8195 |
| AD-1707013.1 | gsascuggauAfCfUfgcagcccgauL96 | 6825 | asdTscgdGgdCugcadGuAfuccagucscsc | 7481 | GGGACUGGAUACUGCAGCCCGAG | 8196 |
| AD-1707014.1 | ascsuggauaCfUfGfcagcccgaguL96 | 6826 | asdCsucdGgdGcugcdAgUfauccaguscsc | 7482 | GGACUGGAUACUGCAGCCCGAGG | 8197 |
| AD-1707015.1 | csusggauacUfGfCfagcccgagguL96 | 6827 | asdCscudCgdGgcugdCaGfuauccagsusc | 7483 | GACUGGAUACUGCAGCCCGAGGG | 8198 |
| AD-1707016.1 | gsusaccagcUfGfAfauuacugcauL96 | 6828 | asdTsgcdAgdTaauudCaGfcugguacscsc | 7484 | GGGUACCAGCUGAAUUACUGCAG | 8199 |
| AD-1707017.1 | usasccagcuGfAfAfuuacugcaguL96 | 6829 | asdCsugdCadGuaaudTcAfgcugguascsc | 7485 | GGUACCAGCUGAAUUACUGCAGU | 8200 |
| AD-1707018.1 | ascscagcugAfAfUfuacugcaguuL96 | 6830 | asdAscudGcdAguaadTuCfagcuggusasc | 7486 | GUACCAGCUGAAUUACUGCAGUG | 8201 |
| AD-1707019.1 | cscsagcugaAfUfUfacugcaguguL96 | 6831 | asdCsacdTgdCaguadAuUfcagcuggsusa | 7487 | UACCAGCUGAAUUACUGCAGUGG | 8202 |
| AD-1707020.1 | csasgcugaaUfUfAfcugcagugguL96 | 6832 | asdCscadCudGcagudAaUfucagcugsgsu | 7488 | ACCAGCUGAAUUACUGCAGUGGG | 8203 |
| AD-1707021.1 | asgscugaauUfAfCfugcaguggguL96 | 6833 | asdCsccdAcdTgcagdTaAfuucagcusgsg | 7489 | CCAGCUGAAUUACUGCAGUGGGC | 8204 |
| AD-1707022.1 | gscsugaauuAfCfUfgcagugggcuL96 | 6834 | asdGsccdCadCugcadGuAfauucagcsusg | 7490 | CAGCUGAAUUACUGCAGUGGGCA | 8205 |
| AD-1707023.1 | csusgaauuaCfUfGfcagugggcauL96 | 6835 | asdTsgcdCcdAcugcdAgUfaauucagscsu | 7491 | AGCUGAAUUACUGCAGUGGGCAG | 8206 |
| AD-1707024.1 | usgsaauuacUfGfCfagugggcaguL96 | 6836 | asdCsugdCcdCacugdCaGfuaauucasgsc | 7492 | GCUGAAUUACUGCAGUGGGCAGU | 8207 |
| AD-1707025.1 | gsasauuacuGfCfAfgugggcaguuL96 | 6837 | asdAscudGcdCcacudGcAfguaauucsasg | 7493 | CUGAAUUACUGCAGUGGGCAGUG | 8208 |
| AD-1707030.1 | ascsugcaguGfGfGfcagugcccuuL96 | 6838 | asdAsggdGcdAcugcdCcAfcugcagusasa | 7494 | UUACUGCAGUGGGCAGUGCCCUC | 8209 |
| AD-1707047.1 | csasggcauuGfCfUfgccucuuucuL96 | 6839 | asdGsaadAgdAggcadGcAfaugccugsgsg | 7495 | CCCAGGCAUUGCUGCCUCUUUCC | 8210 |
| AD-1707048.1 | asgsgcauugCfUfGfccucuuuccuL96 | 6840 | asdGsgadAadGaggcdAgCfaaugccusgsg | 7496 | CCAGGCAUUGCUGCCUCUUUCCA | 8211 |
| AD-1707049.1 | gsgscauugcUfGfCfcucuuuccauL96 | 6841 | asdTsggdAadAgaggdCaGfcaaugccsusg | 7497 | CAGGCAUUGCUGCCUCUUUCCAU | 8212 |
| AD-1707050.1 | gscsauugcuGfCfCfucuuuccauuL96 | 6842 | asdAsugdGadAagagdGcAfgcaaugcscsu | 7498 | AGGCAUUGCUGCCUCUUUCCAUU | 8213 |
| AD-1707051.1 | csasuugcugCfCfUfcuuuccauuuL96 | 6843 | asdAsaudGgdAaagadGgCfagcaaugscsc | 7499 | GGCAUUGCUGCCUCUUUCCAUUC | 8214 |
| AD-1707052.1 | asusugcugcCfUfCfuuuccauucuL96 | 6844 | asdGsaadTgdGaaagdAgGfcagcaausgsc | 7500 | GCAUUGCUGCCUCUUUCCAUUCU | 8215 |
| AD-1707053.1 | ususgcugccUfCfUfuuccauucuuL96 | 6845 | asdAsgadAudGgaaadGaGfgcagcaasusg | 7501 | CAUUGCUGCCUCUUUCCAUUCUG | 8216 |
| AD-1707054.1 | usgscugccuCfUfUfuccauucuguL96 | 6846 | asdCsagdAadTggaadAgAfggcagcasasu | 7502 | AUUGCUGCCUCUUUCCAUUCUGC | 8217 |
| AD-1707055.1 | gscsugccucUfUfUfccauucugcuL96 | 6847 | asdGscadGadAuggadAaGfaggcagcsasa | 7503 | UUGCUGCCUCUUUCCAUUCUGCC | 8218 |
| AD-1707056.1 | csusgccucuUfUfCfcauucugccuL96 | 6848 | asdGsgcdAgdAauggdAaAfgaggcagscsa | 7504 | UGCUGCCUCUUUCCAUUCUGCCG | 8219 |
| AD-1707057.1 | usgsccucuuUfCfCfauucugccguL96 | 6849 | asdCsggdCadGaaugdGaAfagaggcasgsc | 7505 | GCUGCCUCUUUCCAUUCUGCCGU | 8220 |
| AD-1707058.1 | gscscucuuuCfCfAfuucugccguuL96 | 6850 | asdAscgdGcdAgaaudGgAfaagaggcsasg | 7506 | CUGCCUCUUUCCAUUCUGCCGUC | 8221 |
| AD-1707059.1 | cscsucuuucCfAfUfucugccgucuL96 | 6851 | asdGsacdGgdCagaadTgGfaaagaggscsa | 7507 | UGCCUCUUUCCAUUCUGCCGUCU | 8222 |
| AD-1707060.1 | csuscuuuccAfUfUfcugccgucuuL96 | 6852 | asdAsgadCgdGcagadAuGfgaaagagsgsc | 7508 | GCCUCUUUCCAUUCUGCCGUCUU | 8223 |
| AD-1707061.1 | uscsuuuccaUfUfCfugccgucuuuL96 | 6853 | asdAsagdAcdGgcagdAaUfggaaagasgsg | 7509 | CCUCUUUCCAUUCUGCCGUCUUC | 8224 |
| AD-1707062.1 | csusuuccauUfCfUfgccgucuucuL96 | 6854 | asdGsaadGadCggcadGaAfuggaaagsasg | 7510 | CUCUUUCCAUUCUGCCGUCUUCA | 8225 |
| AD-1707063.1 | ususuccauuCfUfGfccgucuucauL96 | 6855 | asdTsgadAgdAcggcdAgAfauggaaasgsa | 7511 | UCUUUCCAUUCUGCCGUCUUCAG | 8226 |
| AD-1707064.1 | ususccauucUfGfCfcgucuucaguL96 | 6856 | asdCsugdAadGacggdCaGfaauggaasasg | 7512 | CUUUCCAUUCUGCCGUCUUCAGC | 8227 |
| AD-1707065.1 | uscscauucuGfCfCfgucuucagcuL96 | 6857 | asdGscudGadAgacgdGcAfgaauggasasa | 7513 | UUUCCAUUCUGCCGUCUUCAGCC | 8228 |
| AD-1707066.1 | cscsauucugCfCfGfucuucagccuL96 | 6858 | asdGsgcdTgdAagacdGgCfagaauggsasa | 7514 | UUCCAUUCUGCCGUCUUCAGCCU | 8229 |
| AD-1707067.1 | csasuucugcCfGfUfcuucagccuuL96 | 6859 | asdAsggdCudGaagadCgGfcagaaugsgsa | 7515 | UCCAUUCUGCCGUCUUCAGCCUC | 8230 |
| AD-1707070.1 | uscsugccguCfUfUfcagccuccuuL96 | 6860 | asdAsggdAgdGcugadAgAfcggcagasasu | 7516 | AUUCUGCCGUCUUCAGCCUCCUC | 8231 |
| AD-1707074.1 | cscsgucuucAfGfCfcuccucaaauL96 | 6861 | asdTsuudGadGgaggdCuGfaagacggscsa | 7517 | UGCCGUCUUCAGCCUCCUCAAAG | 8232 |
| AD-1707075.1 | csgsucuucaGfCfCfuccucaaaguL96 | 6862 | asdCsuudTgdAggagdGcUfgaagacgsgsc | 7518 | GCCGUCUUCAGCCUCCUCAAAGC | 8233 |
| AD-1707076.1 | gsuscuucagCfCfUfccucaaagcuL96 | 6863 | asdGscudTudGaggadGgCfugaagacsgsg | 7519 | CCGUCUUCAGCCUCCUCAAAGCC | 8234 |
| AD-1707077.1 | uscsuucagcCfUfCfcucaaagccuL96 | 6864 | asdGsgcdTudTgaggdAgGfcugaagascsg | 7520 | CGUCUUCAGCCUCCUCAAAGCCA | 8235 |
| AD-1707078.1 | csusucagccUfCfCfucaaagccauL96 | 6865 | asdTsggdCudTugagdGaGfgcugaagsasc | 7521 | GUCUUCAGCCUCCUCAAAGCCAA | 8236 |
| AD-1707080.1 | uscsagccucCfUfCfaaagccaacuL96 | 6866 | asdGsuudGgdCuuugdAgGfaggcugasasg | 7522 | CUUCAGCCUCCUCAAAGCCAACA | 8237 |
| AD-1707081.1 | csasgccuccUfCfAfaagccaacauL96 | 6867 | asdTsgudTgdGcuuudGaGfgaggcugsasa | 7523 | UUCAGCCUCCUCAAAGCCAACAA | 8238 |
| AD-1707082.1 | asgsccuccuCfAfAfagccaacaauL96 | 6868 | asdTsugdTudGgcuudTgAfggaggcusgsa | 7524 | UCAGCCUCCUCAAAGCCAACAAU | 8239 |
| AD-1707083.1 | gscscuccucAfAfAfgccaacaauuL96 | 6869 | asdAsuudGudTggcudTuGfaggaggcsusg | 7525 | CAGCCUCCUCAAAGCCAACAAUC | 8240 |
| AD-1707084.1 | cscsuccucaAfAfGfccaacaaucuL96 | 6870 | asdGsaudTgdTuggcdTuUfgaggaggscsu | 7526 | AGCCUCCUCAAAGCCAACAAUCC | 8241 |
| AD-1707085.1 | csusccucaaAfGfCfcaacaauccuL96 | 6871 | asdGsgadTudGuuggdCuUfugaggagsgsc | 7527 | GCCUCCUCAAAGCCAACAAUCCU | 8242 |
| AD-1707086.1 | uscscucaaaGfCfCfaacaauccuuL96 | 6872 | asdAsggdAudTguugdGcUfuugaggasgsg | 7528 | CCUCCUCAAAGCCAACAAUCCUU | 8243 |
| AD-1707087.1 | cscsucaaagCfCfAfacaauccuuuL96 | 6873 | asdAsagdGadTuguudGgCfuuugaggsasg | 7529 | CUCCUCAAAGCCAACAAUCCUUG | 8244 |
| AD-1707088.1 | csuscaaagcCfAfAfcaauccuuguL96 | 6874 | asdCsaadGgdAuugudTgGfcuuugagsgsa | 7530 | UCCUCAAAGCCAACAAUCCUUGG | 8245 |
| AD-1707092.1 | asasgccaacAfAfUfccuuggccuuL96 | 6875 | asdAsggdCcdAaggadTuGfuuggcuususg | 7531 | CAAAGCCAACAAUCCUUGGCCUG | 8246 |
| AD-1707093.1 | asgsccaacaAfUfCfcuuggccuguL96 | 6876 | asdCsagdGcdCaaggdAuUfguuggcususu | 7532 | AAAGCCAACAAUCCUUGGCCUGC | 8247 |
| AD-1707094.1 | gscscaacaaUfCfCfuuggccugcuL96 | 6877 | asdGscadGgdCcaagdGaUfuguuggcsusu | 7533 | AAGCCAACAAUCCUUGGCCUGCC | 8248 |
| AD-1707095.1 | cscsaacaauCfCfUfuggccugccuL96 | 6878 | asdGsgcdAgdGccaadGgAfuuguuggscsu | 7534 | AGCCAACAAUCCUUGGCCUGCCA | 8249 |
| AD-1707097.1 | asascaauccUfUfGfgccugccaguL96 | 6879 | asdCsugdGcdAggccdAaGfgauuguusgsg | 7535 | CCAACAAUCCUUGGCCUGCCAGU | 8250 |
| AD-1707098.1 | ascsaauccuUfGfGfccugccaguuL96 | 6880 | asdAscudGgdCaggcdCaAfggauugususg | 7536 | CAACAAUCCUUGGCCUGCCAGUA | 8251 |
| AD-1707105.1 | ususggccugCfCfAfguaccuccuuL96 | 6881 | asdAsggdAgdGuacudGgCfaggccaasgsg | 7537 | CCUUGGCCUGCCAGUACCUCCUG | 8252 |
| AD-1707125.1 | gsusugugucCfCfUfacugcccgauL96 | 6882 | asdTscgdGgdCaguadGgGfacacaacsasg | 7538 | CUGUUGUGUCCCUACUGCCCGAA | 8253 |
| AD-1707126.1 | ususguguccCfUfAfcugcccgaauL96 | 6883 | asdTsucdGgdGcagudAgGfgacacaascsa | 7539 | UGUUGUGUCCCUACUGCCCGAAG | 8254 |
| AD-1707127.1 | usgsugucccUfAfCfugcccgaaguL96 | 6884 | asdCsuudCgdGgcagdTaGfggacacasasc | 7540 | GUUGUGUCCCUACUGCCCGAAGG | 8255 |
| AD-1707128.1 | gsusgucccuAfCfUfgcccgaagguL96 | 6885 | asdCscudTcdGggcadGuAfgggacacsasa | 7541 | UUGUGUCCCUACUGCCCGAAGGC | 8256 |
| AD-1707129.1 | usgsucccuaCfUfGfcccgaaggcuL96 | 6886 | asdGsccdTudCgggcdAgUfagggacascsa | 7542 | UGUGUCCCUACUGCCCGAAGGCC | 8257 |
| AD-1707132.1 | uscsucucucCfUfCfuaccuggauuL96 | 6887 | asdAsucdCadGguagdAgGfagagagasgsg | 7543 | CCUCUCUCUCCUCUACCUGGAUC | 8258 |
| AD-1707133.1 | csuscucuccUfCfUfaccuggaucuL96 | 6888 | asdGsaudCcdAgguadGaGfgagagagsasg | 7544 | CUCUCUCUCCUCUACCUGGAUCA | 8259 |
| AD-1707134.1 | uscsucuccuCfUfAfccuggaucauL96 | 6889 | asdTsgadTcdCaggudAgAfggagagasgsa | 7545 | UCUCUCUCCUCUACCUGGAUCAU | 8260 |
| AD-1707135.1 | csuscuccucUfAfCfcuggaucauuL96 | 6890 | asdAsugdAudCcaggdTaGfaggagagsasg | 7546 | CUCUCUCCUCUACCUGGAUCAUA | 8261 |
| AD-1707136.1 | uscsuccucuAfCfCfuggaucauauL96 | 6891 | asdTsaudGadTccagdGuAfgaggagasgsa | 7547 | UCUCUCCUCUACCUGGAUCAUAA | 8262 |
| AD-1707137.1 | csusccucuaCfCfUfggaucauaauL96 | 6892 | asdTsuadTgdAuccadGgUfagaggagsasg | 7548 | CUCUCCUCUACCUGGAUCAUAAU | 8263 |
| AD-1707138.1 | uscscucuacCfUfGfgaucauaauuL96 | 6893 | asdAsuudAudGauccdAgGfuagaggasgsa | 7549 | UCUCCUCUACCUGGAUCAUAAUG | 8264 |
| AD-1707139.1 | cscsucuaccUfGfGfaucauaauguL96 | 6894 | asdCsaudTadTgaucdCaGfguagaggsasg | 7550 | CUCCUCUACCUGGAUCAUAAUGG | 8265 |
| AD-1707140.1 | csuscuaccuGfGfAfucauaaugguL96 | 6895 | asdCscadTudAugaudCcAfgguagagsgsa | 7551 | UCCUCUACCUGGAUCAUAAUGGC | 8266 |
| AD-1707141.1 | uscsuaccugGfAfUfcauaauggcuL96 | 6896 | asdGsccdAudTaugadTcCfagguagasgsg | 7552 | CCUCUACCUGGAUCAUAAUGGCA | 8267 |
| AD-1707142.1 | csusaccuggAfUfCfauaauggcauL96 | 6897 | asdTsgcdCadTuaugdAuCfcagguagsasg | 7553 | CUCUACCUGGAUCAUAAUGGCAA | 8268 |
| AD-1707143.1 | usasccuggaUfCfAfuaauggcaauL96 | 6898 | asdTsugdCcdAuuaudGaUfccagguasgsa | 7554 | UCUACCUGGAUCAUAAUGGCAAU | 8269 |
| AD-1707144.1 | ascscuggauCfAfUfaauggcaauuL96 | 6899 | asdAsuudGcdCauuadTgAfuccaggusasg | 7555 | CUACCUGGAUCAUAAUGGCAAUG | 8270 |
| AD-1707145.1 | cscsuggaucAfUfAfauggcaauguL96 | 6900 | asdCsaudTgdCcauudAuGfauccaggsusa | 7556 | UACCUGGAUCAUAAUGGCAAUGU | 8271 |
| AD-1707146.1 | csusggaucaUfAfAfuggcaauguuL96 | 6901 | asdAscadTudGccaudTaUfgauccagsgsu | 7557 | ACCUGGAUCAUAAUGGCAAUGUG | 8272 |
| AD-1707147.1 | usgsgaucauAfAfUfggcaauguguL96 | 6902 | asdCsacdAudTgccadTuAfugauccasgsg | 7558 | CCUGGAUCAUAAUGGCAAUGUGG | 8273 |
| AD-1707148.1 | gsgsaucauaAfUfGfgcaaugugguL96 | 6903 | asdCscadCadTugccdAuUfaugauccsasg | 7559 | CUGGAUCAUAAUGGCAAUGUGGU | 8274 |
| AD-1707149.1 | gsasucauaaUfGfGfcaaugugguuL96 | 6904 | asdAsccdAcdAuugcdCaUfuaugaucscsa | 7560 | UGGAUCAUAAUGGCAAUGUGGUC | 8275 |
| AD-1707150.1 | asuscauaauGfGfCfaauguggucuL96 | 6905 | asdGsacdCadCauugdCcAfuuaugauscsc | 7561 | GGAUCAUAAUGGCAAUGUGGUCA | 8276 |
| AD-1707151.1 | uscsauaaugGfCfAfauguggucauL96 | 6906 | asdTsgadCcdAcauudGcCfauuaugasusc | 7562 | GAUCAUAAUGGCAAUGUGGUCAA | 8277 |
| AD-1707152.1 | csasuaauggCfAfAfuguggucaauL96 | 6907 | asdTsugdAcdCacaudTgCfcauuaugsasu | 7563 | AUCAUAAUGGCAAUGUGGUCAAG | 8278 |
| AD-1707153.1 | asusaauggcAfAfUfguggucaaguL96 | 6908 | asdCsuudGadCcacadTuGfccauuausgsa | 7564 | UCAUAAUGGCAAUGUGGUCAAGA | 8279 |
| AD-1707154.1 | usasauggcaAfUfGfuggucaagauL96 | 6909 | asdTscudTgdAccacdAuUfgccauuasusg | 7565 | CAUAAUGGCAAUGUGGUCAAGAC | 8280 |
| AD-1707155.1 | asasuggcaaUfGfUfggucaagacuL96 | 6910 | asdGsucdTudGaccadCaUfugccauusasu | 7566 | AUAAUGGCAAUGUGGUCAAGACG | 8281 |
| AD-1707156.1 | asusggcaauGfUfGfgucaagacguL96 | 6911 | asdCsgudCudTgaccdAcAfuugccaususa | 7567 | UAAUGGCAAUGUGGUCAAGACGG | 8282 |
| AD-1707157.1 | usgsgcaaugUfGfGfucaagacgguL96 | 6912 | asdCscgdTcdTugacdCaCfauugccasusu | 7568 | AAUGGCAAUGUGGUCAAGACGGA | 8283 |
| AD-1707158.1 | gsgscaauguGfGfUfcaagacggauL96 | 6913 | asdTsccdGudCuugadCcAfcauugccsasu | 7569 | AUGGCAAUGUGGUCAAGACGGAU | 8284 |
| AD-1707159.1 | gscsaaugugGfUfCfaagacggauuL96 | 6914 | asdAsucdCgdTcuugdAcCfacauugcscsa | 7570 | UGGCAAUGUGGUCAAGACGGAUG | 8285 |
| AD-1707160.1 | csasauguggUfCfAfagacggauguL96 | 6915 | asdCsaudCcdGucuudGaCfcacauugscsc | 7571 | GGCAAUGUGGUCAAGACGGAUGU | 8286 |
| AD-1707161.1 | asasugugguCfAfAfgacggauguuL96 | 6916 | asdAscadTcdCgucudTgAfccacauusgsc | 7572 | GCAAUGUGGUCAAGACGGAUGUG | 8287 |
| AD-1707162.1 | asusguggucAfAfGfacggauguguL96 | 6917 | asdCsacdAudCcgucdTuGfaccacaususg | 7573 | CAAUGUGGUCAAGACGGAUGUGC | 8288 |
| AD-1707163.1 | usgsuggucaAfGfAfcggaugugcuL96 | 6918 | asdGscadCadTccgudCuUfgaccacasusu | 7574 | AAUGUGGUCAAGACGGAUGUGCC | 8289 |
| AD-1707164.1 | gsusggucaaGfAfCfggaugugccuL96 | 6919 | asdGsgcdAcdAuccgdTcUfugaccacsasu | 7575 | AUGUGGUCAAGACGGAUGUGCCA | 8290 |
| AD-1707165.1 | usgsgucaagAfCfGfgaugugccauL96 | 6920 | asdTsggdCadCauccdGuCfuugaccascsa | 7576 | UGUGGUCAAGACGGAUGUGCCAG | 8291 |
| AD-1707166.1 | gsgsucaagaCfGfGfaugugccaguL96 | 6921 | asdCsugdGcdAcaucdCgUfcuugaccsasc | 7577 | GUGGUCAAGACGGAUGUGCCAGA | 8292 |
| AD-1707167.1 | gsuscaagacGfGfAfugugccagauL96 | 6922 | asdTscudGgdCacaudCcGfucuugacscsa | 7578 | UGGUCAAGACGGAUGUGCCAGAU | 8293 |
| AD-1707168.1 | uscsaagacgGfAfUfgugccagauuL96 | 6923 | asdAsucdTgdGcacadTcCfgucuugascsc | 7579 | GGUCAAGACGGAUGUGCCAGAUA | 8294 |
| AD-1707169.1 | csasagacggAfUfGfugccagauauL96 | 6924 | asdTsaudCudGgcacdAuCfcgucuugsasc | 7580 | GUCAAGACGGAUGUGCCAGAUAU | 8295 |
| AD-1707170.1 | asasgacggaUfGfUfgccagauauuL96 | 6925 | asdAsuadTcdTggcadCaUfccgucuusgsa | 7581 | UCAAGACGGAUGUGCCAGAUAUG | 8296 |
| AD-1707171.1 | asgsacggauGfUfGfccagauauguL96 | 6926 | asdCsaudAudCuggcdAcAfuccgucususg | 7582 | CAAGACGGAUGUGCCAGAUAUGG | 8297 |
| AD-1707172.1 | gsascggaugUfGfCfcagauaugguL96 | 6927 | asdCscadTadTcuggdCaCfauccgucsusu | 7583 | AAGACGGAUGUGCCAGAUAUGGU | 8298 |
| AD-1707173.1 | ascsggauguGfCfCfagauaugguuL96 | 6928 | asdAsccdAudAucugdGcAfcauccguscsu | 7584 | AGACGGAUGUGCCAGAUAUGGUG | 8299 |
| AD-1707174.1 | csgsgaugugCfCfAfgauaugguguL96 | 6929 | asdCsacdCadTaucudGgCfacauccgsusc | 7585 | GACGGAUGUGCCAGAUAUGGUGG | 8300 |
| AD-1707176.1 | gsasugugccAfGfAfuauggugguuL96 | 6930 | asdAsccdAcdCauaudCuGfgcacaucscsg | 7586 | CGGAUGUGCCAGAUAUGGUGGUG | 8301 |
| AD-1707177.1 | asusgugccaGfAfUfauggugguguL96 | 6931 | asdCsacdCadCcauadTcUfggcacauscsc | 7587 | GGAUGUGCCAGAUAUGGUGGUGG | 8302 |
| AD-1707178.1 | usgsugccagAfUfAfugguggugguL96 | 6932 | asdCscadCcdAccaudAuCfuggcacasusc | 7588 | GAUGUGCCAGAUAUGGUGGUGGA | 8303 |
| AD-1707179.1 | gsusgccagaUfAfUfggugguggauL96 | 6933 | asdTsccdAcdCaccadTaUfcuggcacsasu | 7589 | AUGUGCCAGAUAUGGUGGUGGAG | 8304 |
| AD-1707180.1 | usgsccagauAfUfGfgugguggaguL96 | 6934 | asdCsucdCadCcaccdAuAfucuggcascsa | 7590 | UGUGCCAGAUAUGGUGGUGGAGG | 8305 |
| AD-1707182.1 | cscsagauauGfGfUfgguggaggcuL96 | 6935 | asdGsccdTcdCaccadCcAfuaucuggscsa | 7591 | UGCCAGAUAUGGUGGUGGAGGCC | 8306 |
| AD-1707189.1 | usgsguggugGfAfGfgccuguggcuL96 | 6936 | asdGsccdAcdAggccdTcCfaccaccasusa | 7592 | UAUGGUGGUGGAGGCCUGUGGCU | 8307 |
| AD-1707190.1 | gsgsugguggAfGfGfccuguggcuuL96 | 6937 | asdAsgcdCadCaggcdCuCfcaccaccsasu | 7593 | AUGGUGGUGGAGGCCUGUGGCUG | 8308 |
| AD-1707197.1 | gsasggccugUfGfGfcugcagcuauL96 | 6938 | asdTsagdCudGcagcdCaCfaggccucscsa | 7594 | UGGAGGCCUGUGGCUGCAGCUAG | 8309 |
| AD-1707200.1 | gscscuguggCfUfGfcagcuagcauL96 | 6939 | asdTsgcdTadGcugcdAgCfcacaggcscsu | 7595 | AGGCCUGUGGCUGCAGCUAGCAA | 8310 |
| AD-1707201.1 | cscsuguggcUfGfCfagcuagcaauL96 | 6940 | asdTsugdCudAgcugdCaGfccacaggscsc | 7596 | GGCCUGUGGCUGCAGCUAGCAAG | 8311 |
| AD-1707202.1 | csusguggcuGfCfAfgcuagcaaguL96 | 6941 | asdCsuudGcdTagcudGcAfgccacagsgsc | 7597 | GCCUGUGGCUGCAGCUAGCAAGA | 8312 |
| AD-1707203.1 | usgsuggcugCfAfGfcuagcaagauL96 | 6942 | asdTscudTgdCuagcdTgCfagccacasgsg | 7598 | CCUGUGGCUGCAGCUAGCAAGAG | 8313 |
| AD-1707204.1 | gsusggcugcAfGfCfuagcaagaguL96 | 6943 | asdCsucdTudGcuagdCuGfcagccacsasg | 7599 | CUGUGGCUGCAGCUAGCAAGAGG | 8314 |
| AD-1707205.1 | usgsgcugcaGfCfUfagcaagagguL96 | 6944 | asdCscudCudTgcuadGcUfgcagccascsa | 7600 | UGUGGCUGCAGCUAGCAAGAGGA | 8315 |
| AD-1707206.1 | gsgscugcagCfUfAfgcaagaggauL96 | 6945 | asdTsccdTcdTugcudAgCfugcagccsasc | 7601 | GUGGCUGCAGCUAGCAAGAGGAC | 8316 |
| AD-1707207.1 | gscsugcagcUfAfGfcaagaggacuL96 | 6946 | asdGsucdCudCuugcdTaGfcugcagcscsa | 7602 | UGGCUGCAGCUAGCAAGAGGACC | 8317 |
| AD-1707208.1 | csusgcagcuAfGfCfaagaggaccuL96 | 6947 | asdGsgudCcdTcuugdCuAfgcugcagscsc | 7603 | GGCUGCAGCUAGCAAGAGGACCU | 8318 |
| AD-1707209.1 | usgscagcuaGfCfAfagaggaccuuL96 | 6948 | asdAsggdTcdCucuudGcUfagcugcasgsc | 7604 | GCUGCAGCUAGCAAGAGGACCUG | 8319 |
| AD-1707210.1 | gscsagcuagCfAfAfgaggaccuguL96 | 6949 | asdCsagdGudCcucudTgCfuagcugcsasg | 7605 | CUGCAGCUAGCAAGAGGACCUGG | 8320 |
| AD-1707211.1 | csasgcuagcAfAfGfaggaccugguL96 | 6950 | asdCscadGgdTccucdTuGfcuagcugscsa | 7606 | UGCAGCUAGCAAGAGGACCUGGG | 8321 |
| AD-1707212.1 | gscsuuuggaGfUfGfaagagaccauL96 | 6951 | asdTsggdTcdTcuucdAcUfccaaagcscsc | 7607 | GGGCUUUGGAGUGAAGAGACCAA | 8322 |
| AD-1707213.1 | csusuuggagUfGfAfagagaccaauL96 | 6952 | asdTsugdGudCucuudCaCfuccaaagscsc | 7608 | GGCUUUGGAGUGAAGAGACCAAG | 8323 |
| AD-1707214.1 | ususuggaguGfAfAfgagaccaaguL96 | 6953 | asdCsuudGgdTcucudTcAfcuccaaasgsc | 7609 | GCUUUGGAGUGAAGAGACCAAGA | 8324 |
| AD-1707216.1 | usgsgagugaAfGfAfgaccaagauuL96 | 6954 | asdAsucdTudGgucudCuUfcacuccasasa | 7610 | UUUGGAGUGAAGAGACCAAGAUG | 8325 |
| AD-1707217.1 | gsgsagugaaGfAfGfaccaagauguL96 | 6955 | asdCsaudCudTggucdTcUfucacuccsasa | 7611 | UUGGAGUGAAGAGACCAAGAUGA | 8326 |
| AD-1707218.1 | gsasgugaagAfGfAfccaagaugauL96 | 6956 | asdTscadTcdTuggudCuCfuucacucscsa | 7612 | UGGAGUGAAGAGACCAAGAUGAA | 8327 |
| AD-1707221.1 | usgsaagagaCfCfAfagaugaaguuL96 | 6957 | asdAscudTcdAucuudGgUfcucuucascsu | 7613 | AGUGAAGAGACCAAGAUGAAGUU | 8328 |
| AD-1707223.1 | asasgagaccAfAfGfaugaaguuuuL96 | 6958 | asdAsaadCudTcaucdTuGfgucucuuscsa | 7614 | UGAAGAGACCAAGAUGAAGUUUC | 8329 |
| AD-1707224.1 | asgsagaccaAfGfAfugaaguuucuL96 | 6959 | asdGsaadAcdTucaudCuUfggucucususc | 7615 | GAAGAGACCAAGAUGAAGUUUCC | 8330 |
| AD-1707225.1 | gsasgaccaaGfAfUfgaaguuuccuL96 | 6960 | asdGsgadAadCuucadTcUfuggucucsusu | 7616 | AAGAGACCAAGAUGAAGUUUCCC | 8331 |
| AD-1707226.1 | asgsaccaagAfUfGfaaguuucccuL96 | 6961 | asdGsggdAadAcuucdAuCfuuggucuscsu | 7617 | AGAGACCAAGAUGAAGUUUCCCA | 8332 |
| AD-1707227.1 | gsasccaagaUfGfAfaguuucccauL96 | 6962 | asdTsggdGadAacuudCaUfcuuggucsusc | 7618 | GAGACCAAGAUGAAGUUUCCCAG | 8333 |
| AD-1707228.1 | ascscaagauGfAfAfguuucccaguL96 | 6963 | asdCsugdGgdAaacudTcAfucuugguscsu | 7619 | AGACCAAGAUGAAGUUUCCCAGG | 8334 |
| AD-1707229.1 | cscsaagaugAfAfGfuuucccagguL96 | 6964 | asdCscudGgdGaaacdTuCfaucuuggsusc | 7620 | GACCAAGAUGAAGUUUCCCAGGC | 8335 |
| AD-1707230.1 | csasagaugaAfGfUfuucccaggcuL96 | 6965 | asdGsccdTgdGgaaadCuUfcaucuugsgsu | 7621 | ACCAAGAUGAAGUUUCCCAGGCA | 8336 |
| AD-1707231.1 | asasgaugaaGfUfUfucccaggcauL96 | 6966 | asdTsgcdCudGggaadAcUfucaucuusgsg | 7622 | CCAAGAUGAAGUUUCCCAGGCAC | 8337 |
| AD-1707232.1 | asgsaugaagUfUfUfcccaggcacuL96 | 6967 | asdGsugdCcdTgggadAaCfuucaucususg | 7623 | CAAGAUGAAGUUUCCCAGGCACA | 8338 |
| AD-1707233.1 | gsasugaaguUfUfCfccaggcacauL96 | 6968 | asdTsgudGcdCugggdAaAfcuucaucsusu | 7624 | AAGAUGAAGUUUCCCAGGCACAG | 8339 |
| AD-1707234.1 | asusgaaguuUfCfCfcaggcacaguL96 | 6969 | asdCsugdTgdCcuggdGaAfacuucauscsu | 7625 | AGAUGAAGUUUCCCAGGCACAGG | 8340 |
| AD-1707236.1 | gsasaguuucCfCfAfggcacaggguL96 | 6970 | asdCsccdTgdTgccudGgGfaaacuucsasu | 7626 | AUGAAGUUUCCCAGGCACAGGGC | 8341 |
| AD-1707237.1 | asasguuuccCfAfGfgcacagggcuL96 | 6971 | asdGsccdCudGugccdTgGfgaaacuuscsa | 7627 | UGAAGUUUCCCAGGCACAGGGCA | 8342 |
| AD-1707239.1 | gsusuucccaGfGfCfacagggcauuL96 | 6972 | asdAsugdCcdCugugdCcUfgggaaacsusu | 7628 | AAGUUUCCCAGGCACAGGGCAUC | 8343 |
| AD-1707240.1 | ususucccagGfCfAfcagggcaucuL96 | 6973 | asdGsaudGcdCcugudGcCfugggaaascsu | 7629 | AGUUUCCCAGGCACAGGGCAUCU | 8344 |
| AD-1707241.1 | ususcccaggCfAfCfagggcaucuuL96 | 6974 | asdAsgadTgdCccugdTgCfcugggaasasc | 7630 | GUUUCCCAGGCACAGGGCAUCUG | 8345 |
| AD-1707242.1 | uscsccaggcAfCfAfgggcaucuguL96 | 6975 | asdCsagdAudGcccudGuGfccugggasasa | 7631 | UUUCCCAGGCACAGGGCAUCUGU | 8346 |
| AD-1707275.1 | csasacccaaCfAfAfccaccuggcuL96 | 6976 | asdGsccdAgdGuggudTgUfuggguugsgsg | 7632 | CCCAACCCAACAACCACCUGGCA | 8347 |
| AD-1707276.1 | asascccaacAfAfCfcaccuggcauL96 | 6977 | asdTsgcdCadGguggdTuGfuuggguusgsg | 7633 | CCAACCCAACAACCACCUGGCAA | 8348 |
| AD-1707277.1 | ascsccaacaAfCfCfaccuggcaauL96 | 6978 | asdTsugdCcdAggugdGuUfguugggususg | 7634 | CAACCCAACAACCACCUGGCAAU | 8349 |
| AD-1707278.1 | cscscaacaaCfCfAfccuggcaauuL96 | 6979 | asdAsuudGcdCaggudGgUfuguugggsusu | 7635 | AACCCAACAACCACCUGGCAAUA | 8350 |
| AD-1707279.1 | cscsaacaacCfAfCfcuggcaauauL96 | 6980 | asdTsaudTgdCcaggdTgGfuuguuggsgsu | 7636 | ACCCAACAACCACCUGGCAAUAU | 8351 |
| AD-1707280.1 | csasacaaccAfCfCfuggcaauauuL96 | 6981 | asdAsuadTudGccagdGuGfguuguugsgsg | 7637 | CCCAACAACCACCUGGCAAUAUG | 8352 |
| AD-1707281.1 | asascaaccaCfCfUfggcaauauguL96 | 6982 | asdCsaudAudTgccadGgUfgguuguusgsg | 7638 | CCAACAACCACCUGGCAAUAUGA | 8353 |
| AD-1707282.1 | ascsaaccacCfUfGfgcaauaugauL96 | 6983 | asdTscadTadTugccdAgGfugguugususg | 7639 | CAACAACCACCUGGCAAUAUGAC | 8354 |
| AD-1707283.1 | csasaccaccUfGfGfcaauaugacuL96 | 6984 | asdGsucdAudAuugcdCaGfgugguugsusu | 7640 | AACAACCACCUGGCAAUAUGACU | 8355 |
| AD-1707284.1 | asasccaccuGfGfCfaauaugacuuL96 | 6985 | asdAsgudCadTauugdCcAfggugguusgsu | 7641 | ACAACCACCUGGCAAUAUGACUC | 8356 |
| AD-1707285.1 | ascscaccugGfCfAfauaugacucuL96 | 6986 | asdGsagdTcdAuauudGcCfagguggususg | 7642 | CAACCACCUGGCAAUAUGACUCA | 8357 |
| AD-1707286.1 | cscsaccuggCfAfAfuaugacucauL96 | 6987 | asdTsgadGudCauaudTgCfcagguggsusu | 7643 | AACCACCUGGCAAUAUGACUCAC | 8358 |
| AD-1707287.1 | csasccuggcAfAfUfaugacucacuL96 | 6988 | asdGsugdAgdTcauadTuGfccaggugsgsu | 7644 | ACCACCUGGCAAUAUGACUCACU | 8359 |
| AD-1707288.1 | ascscuggcaAfUfAfugacucacuuL96 | 6989 | asdAsgudGadGucaudAuUfgccaggusgsg | 7645 | CCACCUGGCAAUAUGACUCACUU | 8360 |
| AD-1707289.1 | cscsuggcaaUfAfUfgacucacuuuL96 | 6990 | asdAsagdTgdAgucadTaUfugccaggsusg | 7646 | CACCUGGCAAUAUGACUCACUUG | 8361 |
| AD-1707290.1 | csusggcaauAfUfGfacucacuuguL96 | 6991 | asdCsaadGudGagucdAuAfuugccagsgsu | 7647 | ACCUGGCAAUAUGACUCACUUGA | 8362 |
| AD-1707291.1 | usgsgcaauaUfGfAfcucacuugauL96 | 6992 | asdTscadAgdTgagudCaUfauugccasgsg | 7648 | CCUGGCAAUAUGACUCACUUGAC | 8363 |
| AD-1707292.1 | gsgscaauauGfAfCfucacuugacuL96 | 6993 | asdGsucdAadGugagdTcAfuauugccsasg | 7649 | CUGGCAAUAUGACUCACUUGACC | 8364 |
| AD-1707293.1 | gscsaauaugAfCfUfcacuugaccuL96 | 6994 | asdGsgudCadAgugadGuCfauauugcscsa | 7650 | UGGCAAUAUGACUCACUUGACCC | 8365 |
| AD-1707299.1 | gsgsacccaaAfUfGfggcacuuucuL96 | 6995 | asdGsaadAgdTgcccdAuUfuggguccscsa | 7651 | UGGGACCCAAAUGGGCACUUUCU | 8366 |
| AD-1707301.1 | ascsccaaauGfGfGfcacuuucuuuL96 | 6996 | asdAsagdAadAgugcdCcAfuuuggguscsc | 7652 | GGACCCAAAUGGGCACUUUCUUG | 8367 |
| AD-1707302.1 | cscscaaaugGfGfCfacuuucuuguL96 | 6997 | asdCsaadGadAagugdCcCfauuugggsusc | 7653 | GACCCAAAUGGGCACUUUCUUGU | 8368 |
| AD-1707303.1 | cscsaaauggGfCfAfcuuucuuguuL96 | 6998 | asdAscadAgdAaagudGcCfcauuuggsgsu | 7654 | ACCCAAAUGGGCACUUUCUUGUC | 8369 |
| AD-1707304.1 | csasaaugggCfAfCfuuucuugucuL96 | 6999 | asdGsacdAadGaaagdTgCfccauuugsgsg | 7655 | CCCAAAUGGGCACUUUCUUGUCU | 8370 |
| AD-1707305.1 | asasaugggcAfCfUfuucuugucuuL96 | 7000 | asdAsgadCadAgaaadGuGfcccauuusgsg | 7656 | CCAAAUGGGCACUUUCUUGUCUG | 8371 |
| AD-1707306.1 | asasugggcaCfUfUfucuugucuguL96 | 7001 | asdCsagdAcdAagaadAgUfgcccauususg | 86 | CAAAUGGGCACUUUCUUGUCUGA | 8372 |
| AD-1707307.1 | asusgggcacUfUfUfcuugucugauL96 | 7002 | asdTscadGadCaagadAaGfugcccaususu | 7657 | AAAUGGGCACUUUCUUGUCUGAG | 8373 |
| AD-1707308.1 | usgsggcacuUfUfCfuugucugaguL96 | 7003 | asdCsucdAgdAcaagdAaAfgugcccasusu | 7658 | AAUGGGCACUUUCUUGUCUGAGA | 8374 |
| AD-1707309.1 | gsgsgcacuuUfCfUfugucugagauL96 | 7004 | asdTscudCadGacaadGaAfagugcccsasu | 7659 | AUGGGCACUUUCUUGUCUGAGAC | 8375 |
| AD-1707310.1 | gsgscacuuuCfUfUfgucugagacuL96 | 7005 | asdGsucdTcdAgacadAgAfaagugccscsa | 7660 | UGGGCACUUUCUUGUCUGAGACU | 8376 |
| AD-1707311.1 | gscsacuuucUfUfGfucugagacuuL96 | 7006 | asdAsgudCudCagacdAaGfaaagugcscsc | 7661 | GGGCACUUUCUUGUCUGAGACUC | 8377 |
| AD-1707312.1 | csascuuucuUfGfUfcugagacucuL96 | 7007 | asdGsagdTcdTcagadCaAfgaaagugscsc | 7662 | GGCACUUUCUUGUCUGAGACUCU | 8378 |
| AD-1707313.1 | ascsuuucuuGfUfCfugagacucuuL96 | 7008 | asdAsgadGudCucagdAcAfagaaagusgsc | 7663 | GCACUUUCUUGUCUGAGACUCUG | 8379 |
| AD-1707314.1 | csusuucuugUfCfUfgagacucuguL96 | 7009 | asdCsagdAgdTcucadGaCfaagaaagsusg | 7664 | CACUUUCUUGUCUGAGACUCUGG | 8380 |
| AD-1707315.1 | ususucuuguCfUfGfagacucugguL96 | 7010 | asdCscadGadGucucdAgAfcaagaaasgsu | 7665 | ACUUUCUUGUCUGAGACUCUGGC | 8381 |
| AD-1707316.1 | ususcuugucUfGfAfgacucuggcuL96 | 7011 | asdGsccdAgdAgucudCaGfacaagaasasg | 7666 | CUUUCUUGUCUGAGACUCUGGCU | 8382 |
| AD-1707317.1 | uscsuugucuGfAfGfacucuggcuuL96 | 7012 | asdAsgcdCadGagucdTcAfgacaagasasa | 7667 | UUUCUUGUCUGAGACUCUGGCUU | 8383 |
| AD-1707318.1 | csusugucugAfGfAfcucuggcuuuL96 | 7013 | asdAsagdCcdAgagudCuCfagacaagsasa | 7668 | UUCUUGUCUGAGACUCUGGCUUA | 8384 |
| AD-1707337.1 | usasuuccagGfUfUfggcugauguuL96 | 7014 | asdAscadTcdAgccadAcCfuggaauasasg | 7669 | CUUAUUCCAGGUUGGCUGAUGUG | 8385 |
| AD-1707338.1 | asusuccaggUfUfGfgcugauguguL96 | 7015 | asdCsacdAudCagccdAaCfcuggaausasa | 7670 | UUAUUCCAGGUUGGCUGAUGUGU | 8386 |
| AD-1707339.1 | ususccagguUfGfGfcugauguguuL96 | 7016 | asdAscadCadTcagcdCaAfccuggaasusa | 7671 | UAUUCCAGGUUGGCUGAUGUGUU | 8387 |
| AD-1707340.1 | uscscagguuGfGfCfugauguguuuL96 | 7017 | asdAsacdAcdAucagdCcAfaccuggasasu | 7672 | AUUCCAGGUUGGCUGAUGUGUUG | 8388 |
| AD-1707341.1 | cscsagguugGfCfUfgauguguuguL96 | 7018 | asdCsaadCadCaucadGcCfaaccuggsasa | 7673 | UUCCAGGUUGGCUGAUGUGUUGG | 8389 |
| AD-1707342.1 | csasgguuggCfUfGfauguguugguL96 | 7019 | asdCscadAcdAcaucdAgCfcaaccugsgsa | 7674 | UCCAGGUUGGCUGAUGUGUUGGG | 8390 |
| AD-1707388.1 | gsasuuuccuGfCfCfcuaaguccuuL96 | 7020 | asdAsggdAcdTuaggdGcAfggaaaucsasu | 7675 | AUGAUUUCCUGCCCUAAGUCCUG | 8391 |
| AD-1707390.1 | ususuccugcCfCfUfaaguccuguuL96 | 7021 | asdAscadGgdAcuuadGgGfcaggaaasusc | 7676 | GAUUUCCUGCCCUAAGUCCUGUG | 8392 |
| AD-1707411.1 | asgsaagaugUfCfAfgggacuagguL96 | 7022 | asdCscudAgdTcccudGaCfaucuucuscsa | 7677 | UGAGAAGAUGUCAGGGACUAGGG | 8393 |
| AD-1707412.1 | gsasagauguCfAfGfggacuaggguL96 | 7023 | asdCsccdTadGucccdTgAfcaucuucsusc | 7678 | GAGAAGAUGUCAGGGACUAGGGA | 8394 |
| AD-1707415.1 | asasgaugucAfGfGfgacuagggauL96 | 7024 | asdTsccdCudAguccdCuGfacaucuuscsu | 7679 | AGAAGAUGUCAGGGACUAGGGAG | 8395 |
| AD-1707416.1 | asgsaugucaGfGfGfacuagggaguL96 | 7025 | asdCsucdCcdTagucdCcUfgacaucususc | 7680 | GAAGAUGUCAGGGACUAGGGAGG | 8396 |
| AD-1707417.1 | gsasugucagGfGfAfcuagggagguL96 | 7026 | asdCscudCcdCuagudCcCfugacaucsusu | 7681 | AAGAUGUCAGGGACUAGGGAGGG | 8397 |
| AD-1707418.1 | asusgucaggGfAfCfuagggaggguL96 | 7027 | asdCsccdTcdCcuagdTcCfcugacauscsu | 7682 | AGAUGUCAGGGACUAGGGAGGGA | 8398 |
| AD-1707466.1 | gsasggaggaAfGfCfagauagauguL96 | 7028 | asdCsaudCudAucugdCuUfccuccucscsc | 7683 | GGGAGGAGGAAGCAGAUAGAUGG | 8399 |
| AD-1707467.1 | asgsgaggaaGfCfAfgauagaugguL96 | 7029 | asdCscadTcdTaucudGcUfuccuccuscsc | 7684 | GGAGGAGGAAGCAGAUAGAUGGU | 8400 |
| AD-1707468.1 | gsgsaggaagCfAfGfauagaugguuL96 | 7030 | asdAsccdAudCuaucdTgCfuuccuccsusc | 7685 | GAGGAGGAAGCAGAUAGAUGGUC | 8401 |
| AD-1707469.1 | gsasggaagcAfGfAfuagauggucuL96 | 7031 | asdGsacdCadTcuaudCuGfcuuccucscsu | 7686 | AGGAGGAAGCAGAUAGAUGGUCC | 8402 |
| AD-1707470.1 | asgsgaagcaGfAfUfagaugguccuL96 | 7032 | asdGsgadCcdAucuadTcUfgcuuccuscsc | 7687 | GGAGGAAGCAGAUAGAUGGUCCA | 8403 |
| AD-1707471.1 | gsgsaagcagAfUfAfgaugguccauL96 | 7033 | asdTsggdAcdCaucudAuCfugcuuccsusc | 7688 | GAGGAAGCAGAUAGAUGGUCCAG | 8404 |
| AD-1707472.1 | gsasagcagaUfAfGfaugguccaguL96 | 7034 | asdCsugdGadCcaucdTaUfcugcuucscsu | 7689 | AGGAAGCAGAUAGAUGGUCCAGC | 8405 |
| AD-1707473.1 | asasgcagauAfGfAfugguccagcuL96 | 7035 | asdGscudGgdAccaudCuAfucugcuuscsc | 7690 | GGAAGCAGAUAGAUGGUCCAGCA | 8406 |
| AD-1707474.1 | asgscagauaGfAfUfgguccagcauL96 | 7036 | asdTsgcdTgdGaccadTcUfaucugcususc | 7691 | GAAGCAGAUAGAUGGUCCAGCAG | 8407 |
| AD-1707475.1 | gscsagauagAfUfGfguccagcaguL96 | 7037 | asdCsugdCudGgaccdAuCfuaucugcsusu | 7692 | AAGCAGAUAGAUGGUCCAGCAGG | 8408 |
| AD-1707476.1 | csasgauagaUfGfGfuccagcagguL96 | 7038 | asdCscudGcdTggacdCaUfcuaucugscsu | 7693 | AGCAGAUAGAUGGUCCAGCAGGC | 8409 |
| AD-1707477.1 | asgsauagauGfGfUfccagcaggcuL96 | 7039 | asdGsccdTgdCuggadCcAfucuaucusgsc | 7694 | GCAGAUAGAUGGUCCAGCAGGCU | 8410 |
| AD-1707478.1 | gsasuagaugGfUfCfcagcaggcuuL96 | 7040 | asdAsgcdCudGcuggdAcCfaucuaucsusg | 7695 | CAGAUAGAUGGUCCAGCAGGCUU | 8411 |
| AD-1707479.1 | asusagauggUfCfCfagcaggcuuuL96 | 7041 | asdAsagdCcdTgcugdGaCfcaucuauscsu | 7696 | AGAUAGAUGGUCCAGCAGGCUUG | 8412 |
| AD-1707480.1 | usasgaugguCfCfAfgcaggcuuguL96 | 7042 | asdCsaadGcdCugcudGgAfccaucuasusc | 7697 | GAUAGAUGGUCCAGCAGGCUUGA | 8413 |
| AD-1707481.1 | asgsauggucCfAfGfcaggcuugauL96 | 7043 | asdTscadAgdCcugcdTgGfaccaucusasu | 7698 | AUAGAUGGUCCAGCAGGCUUGAA | 8414 |
| AD-1707482.1 | gsasugguccAfGfCfaggcuugaauL96 | 7044 | asdTsucdAadGccugdCuGfgaccaucsusa | 7699 | UAGAUGGUCCAGCAGGCUUGAAG | 8415 |
| AD-1707483.1 | asusgguccaGfCfAfggcuugaaguL96 | 7045 | asdCsuudCadAgccudGcUfggaccauscsu | 7700 | AGAUGGUCCAGCAGGCUUGAAGC | 8416 |
| AD-1707484.1 | usgsguccagCfAfGfgcuugaagcuL96 | 7046 | asdGscudTcdAagccdTgCfuggaccasusc | 7701 | GAUGGUCCAGCAGGCUUGAAGCA | 8417 |
| AD-1707485.1 | gsgsuccagcAfGfGfcuugaagcauL96 | 7047 | asdTsgcdTudCaagcdCuGfcuggaccsasu | 7702 | AUGGUCCAGCAGGCUUGAAGCAG | 8418 |
| AD-1707486.1 | gsusccagcaGfGfCfuugaagcaguL96 | 7048 | asdCsugdCudTcaagdCcUfgcuggacscsa | 7703 | UGGUCCAGCAGGCUUGAAGCAGG | 8419 |
| AD-1707553.1 | asasggucaaGfAfGfggagauggguL96 | 7049 | asdCsccdAudCucccdTcUfugaccuuscsc | 7704 | GGAAGGUCAAGAGGGAGAUGGGC | 8420 |
| AD-1707554.1 | asgsgucaagAfGfGfgagaugggcuL96 | 7050 | asdGsccdCadTcuccdCuCfuugaccususc | 7705 | GAAGGUCAAGAGGGAGAUGGGCA | 8421 |
| AD-1707555.1 | gsgsucaagaGfGfGfagaugggcauL96 | 7051 | asdTsgcdCcdAucucdCcUfcuugaccsusu | 7706 | AAGGUCAAGAGGGAGAUGGGCAA | 8422 |
| AD-1707556.1 | gsuscaagagGfGfAfgaugggcaauL96 | 7052 | asdTsugdCcdCaucudCcCfucuugacscsu | 7707 | AGGUCAAGAGGGAGAUGGGCAAG | 8423 |
| AD-1707557.1 | uscsaagaggGfAfGfaugggcaaguL96 | 7053 | asdCsuudGcdCcaucdTcCfcucuugascsc | 7708 | GGUCAAGAGGGAGAUGGGCAAGG | 8424 |
| AD-1707577.1 | gscsgcugagGfGfAfggaugcuuauL96 | 7054 | asdTsaadGcdAuccudCcCfucagcgcscsu | 7709 | AGGCGCUGAGGGAGGAUGCUUAG | 8425 |
| AD-1707578.1 | csgscugaggGfAfGfgaugcuuaguL96 | 7055 | asdCsuadAgdCauccdTcCfcucagcgscsc | 7710 | GGCGCUGAGGGAGGAUGCUUAGG | 8426 |
| AD-1707605.1 | gscsacuaagCfCfUfaagaaguucuL96 | 7056 | asdGsaadCudTcuuadGgCfuuagugcscsu | 7711 | AGGCACUAAGCCUAAGAAGUUCC | 8427 |
| AD-1707606.1 | csascuaagcCfUfAfagaaguuccuL96 | 7057 | asdGsgadAcdTucuudAgGfcuuagugscsc | 7712 | GGCACUAAGCCUAAGAAGUUCCC | 8428 |
| AD-1707623.1 | ascsaggaccCfAfCfugggagacauL96 | 7058 | asdTsgudCudCccagdTgGfguccuguscsc | 7713 | GGACAGGACCCACUGGGAGACAA | 8429 |
| AD-1707624.1 | csasggacccAfCfUfgggagacaauL96 | 7059 | asdTsugdTcdTcccadGuGfgguccugsusc | 7714 | GACAGGACCCACUGGGAGACAAG | 8430 |
| AD-1707625.1 | asgsgacccaCfUfGfggagacaaguL96 | 7060 | asdCsuudGudCucccdAgUfggguccusgsu | 7715 | ACAGGACCCACUGGGAGACAAGC | 8431 |
| AD-1707626.1 | gsgsacccacUfGfGfgagacaagcuL96 | 7061 | asdGscudTgdTcuccdCaGfuggguccsusg | 7716 | CAGGACCCACUGGGAGACAAGCA | 8432 |
| AD-1707627.1 | gsascccacuGfGfGfagacaagcauL96 | 7062 | asdTsgcdTudGucucdCcAfgugggucscsu | 7717 | AGGACCCACUGGGAGACAAGCAU | 8433 |
| AD-1707628.1 | ascsccacugGfGfAfgacaagcauuL96 | 7063 | asdAsugdCudTgucudCcCfaguggguscsc | 7718 | GGACCCACUGGGAGACAAGCAUU | 8434 |
| AD-1707629.1 | cscscacuggGfAfGfacaagcauuuL96 | 7064 | asdAsaudGcdTugucdTcCfcagugggsusc | 7719 | GACCCACUGGGAGACAAGCAUUU | 8435 |
| AD-1707630.1 | cscsacugggAfGfAfcaagcauuuuL96 | 7065 | asdAsaadTgdCuugudCuCfccaguggsgsu | 7720 | ACCCACUGGGAGACAAGCAUUUA | 8436 |
| AD-1707631.1 | csascugggaGfAfCfaagcauuuauL96 | 7066 | asdTsaadAudGcuugdTcUfcccagugsgsg | 7721 | CCCACUGGGAGACAAGCAUUUAU | 8437 |
| AD-1707632.1 | ascsugggagAfCfAfagcauuuauuL96 | 7067 | asdAsuadAadTgcuudGuCfucccagusgsg | 7722 | CCACUGGGAGACAAGCAUUUAUA | 8438 |
| AD-1707633.1 | csusgggagaCfAfAfgcauuuauauL96 | 7068 | asdTsaudAadAugcudTgUfcucccagsusg | 7723 | CACUGGGAGACAAGCAUUUAUAC | 8439 |
| AD-1707634.1 | usgsggagacAfAfGfcauuuauacuL96 | 7069 | asdGsuadTadAaugcdTuGfucucccasgsu | 7724 | ACUGGGAGACAAGCAUUUAUACU | 8440 |
| AD-1707636.1 | gsgsagacaaGfCfAfuuuauacuuuL96 | 7070 | asdAsagdTadTaaaudGcUfugucuccscsa | 7725 | UGGGAGACAAGCAUUUAUACUUU | 8441 |
| AD-1707637.1 | gsasgacaagCfAfUfuuauacuuuuL96 | 7071 | asdAsaadGudAuaaadTgCfuugucucscsc | 7726 | GGGAGACAAGCAUUUAUACUUUC | 8442 |
| AD-1707638.1 | asgsacaagcAfUfUfuauacuuucuL96 | 7072 | asdGsaadAgdTauaadAuGfcuugucuscsc | 7727 | GGAGACAAGCAUUUAUACUUUCU | 8443 |
| AD-1707639.1 | gsascaagcaUfUfUfauacuuucuuL96 | 7073 | asdAsgadAadGuauadAaUfgcuugucsusc | 88 | GAGACAAGCAUUUAUACUUUCUU | 8444 |
| AD-1707640.1 | ascsaagcauUfUfAfuacuuucuuuL96 | 7074 | asdAsagdAadAguaudAaAfugcuuguscsu | 90 | AGACAAGCAUUUAUACUUUCUUU | 8445 |
| AD-1707641.1 | csasagcauuUfAfUfacuuucuuuuL96 | 7075 | asdAsaadGadAaguadTaAfaugcuugsusc | 7728 | GACAAGCAUUUAUACUUUCUUUC | 8446 |
| AD-1708118.1 | cscsagacAfuGfAfGfcugugaggguL96 | 7076 | asCfsccdTc(Agn)cagcucAfuGfucuggscsu | 7729 | AGCCAGACAUGAGCUGUGAGGGU | 7945 |
| AD-1708120.1 | asgsacauGfaGfCfUfgugagggucuL96 | 7077 | asGfsacdCc(Tgn)cacagcUfcAfugucusgsg | 7730 | CCAGACAUGAGCUGUGAGGGUCA | 7946 |
| AD-1708124.1 | asusgagcUfgUfGfAfgggucaagcuL96 | 7078 | asGfscudTg(Agn)cccucaCfaGfcucausgsu | 7731 | ACAUGAGCUGUGAGGGUCAAGCA | 7950 |
| AD-1708126.1 | gsasgcugUfgAfGfGfgucaagcacuL96 | 7079 | asGfsugdCu(Tgn)gacccuCfaCfagcucsasu | 7732 | AUGAGCUGUGAGGGUCAAGCACA | 7952 |
| AD-1708127.1 | asgscuguGfaGfGfGfucaagcacauL96 | 7080 | asUfsgudGc(Tgn)ugacccUfcAfcagcuscsa | 7733 | UGAGCUGUGAGGGUCAAGCACAG | 7953 |
| AD-1708130.1 | usgsugagGfgUfCfAfagcacagcuuL96 | 7081 | asAfsgcdTg(Tgn)gcuugaCfcCfucacasgsc | 7734 | GCUGUGAGGGUCAAGCACAGCUA | 7956 |
| AD-1708132.1 | usgsagggUfcAfAfGfcacagcuauuL96 | 7082 | asAfsuadGc(Tgn)gugcuuGfaCfccucascsa | 7735 | UGUGAGGGUCAAGCACAGCUAUC | 7958 |
| AD-1708135.1 | gsgsgucaAfgCfAfCfagcuauccauL96 | 7083 | asUfsggdAu(Agn)gcugugCfuUfgacccsusc | 7736 | GAGGGUCAAGCACAGCUAUCCAU | 7961 |
| AD-1708140.1 | asasgcacAfgCfUfAfuccaucagauL96 | 7084 | asUfscudGa(Tgn)ggauagCfuGfugcuusgsa | 7737 | UCAAGCACAGCUAUCCAUCAGAU | 7963 |
| AD-1708143.1 | csascagcUfaUfCfCfaucagaugauL96 | 7085 | asUfscadTc(Tgn)gauggaUfaGfcugugscsu | 7738 | AGCACAGCUAUCCAUCAGAUGAU | 7965 |
| AD-1708145.1 | csasgcuaUfcCfAfUfcagaugaucuL96 | 7086 | asGfsaudCa(Tgn)cugaugGfaUfagcugsusg | 7739 | CACAGCUAUCCAUCAGAUGAUCU | 7967 |
| AD-1708146.1 | asgscuauCfcAfUfCfagaugaucuuL96 | 7087 | asAfsgadTc(Agn)ucugauGfgAfuagcusgsu | 7740 | ACAGCUAUCCAUCAGAUGAUCUA | 7968 |
| AD-1708148.1 | csusauccAfuCfAfGfaugaucuacuL96 | 7088 | asGfsuadGa(Tgn)caucugAfuGfgauagscsu | 7741 | AGCUAUCCAUCAGAUGAUCUACU | 7970 |
| AD-1708149.1 | usasuccaUfcAfGfAfugaucuacuuL96 | 7089 | asAfsgudAg(Agn)ucaucuGfaUfggauasgsc | 7742 | GCUAUCCAUCAGAUGAUCUACUU | 7971 |
| AD-1708151.1 | uscscaucAfgAfUfGfaucuacuuuuL96 | 7090 | asAfsaadGu(Agn)gaucauCfuGfauggasusa | 7743 | UAUCCAUCAGAUGAUCUACUUUC | 7973 |
| AD-1708152.1 | cscsaucaGfaUfGfAfucuacuuucuL96 | 7091 | asGfsaadAg(Tgn)agaucaUfcUfgauggsasu | 7744 | AUCCAUCAGAUGAUCUACUUUCA | 7974 |
| AD-1708156.1 | csasgaugAfuCfUfAfcuuucagccuL96 | 7092 | asGfsgcdTg(Agn)aaguagAfuCfaucugsasu | 7745 | AUCAGAUGAUCUACUUUCAGCCU | 7978 |
| AD-1708158.1 | gsasugauCfuAfCfUfuucagccuuuL96 | 7093 | asAfsagdGc(Tgn)gaaaguAfgAfucaucsusg | 7746 | CAGAUGAUCUACUUUCAGCCUUC | 7980 |
| AD-1708163.1 | uscsuacuUfuCfAfGfccuuccugauL96 | 7094 | asUfscadGg(Agn)aggcugAfaAfguagasusc | 7747 | GAUCUACUUUCAGCCUUCCUGAG | 7985 |
| AD-1708166.1 | ascsuuucAfgCfCfUfuccugagucuL96 | 7095 | asGfsacdTc(Agn)ggaaggCfuGfaaagusasg | 7748 | CUACUUUCAGCCUUCCUGAGUCC | 7988 |
| AD-1708170.1 | uscsagccUfuCfCfUfgagucccaguL96 | 7096 | asCfsugdGg(Agn)cucaggAfaGfgcugasasa | 7749 | UUUCAGCCUUCCUGAGUCCCAGG | 8447 |
| AD-1708220.1 | gsusggcaGfuGfGfUfgucugcuguuL96 | 405 | asAfscadGc(Agn)gacaccAfcUfgccacsasc | 7750 | GUGUGGCAGUGGUGUCUGCUGUC | 7992 |
| AD-1708223.1 | gscsagugGfuGfUfCfugcugucacuL96 | 7097 | asGfsugdAc(Agn)gcagacAfcCfacugcscsa | 7751 | UGGCAGUGGUGUCUGCUGUCACU | 7995 |
| AD-1708225.1 | asgsugguGfuCfUfGfcugucacuguL96 | 7098 | asCfsagdTg(Agn)cagcagAfcAfccacusgsc | 7752 | GCAGUGGUGUCUGCUGUCACUGU | 7997 |
| AD-1708227.1 | usgsguguCfuGfCfUfgucacuguguL96 | 7099 | asCfsacdAg(Tgn)gacagcAfgAfcaccascsu | 7753 | AGUGGUGUCUGCUGUCACUGUGC | 7999 |
| AD-1708245.1 | gsascucaAfcAfGfAfcggagcaacuL96 | 7100 | asGfsuudGc(Tgn)ccgucuGfuUfgagucsusg | 7754 | CAGACUCAACAGACGGAGCAACU | 8003 |
| AD-1708248.1 | uscsaacaGfaCfGfGfagcaacugcuL96 | 7101 | asGfscadGu(Tgn)gcuccgUfcUfguugasgsu | 7755 | ACUCAACAGACGGAGCAACUGCC | 8006 |
| AD-1708249.1 | csasacagAfcGfGfAfgcaacugccuL96 | 7102 | asGfsgcdAg(Tgn)ugcuccGfuCfuguugsasg | 7756 | CUCAACAGACGGAGCAACUGCCA | 8007 |
| AD-1708251.1 | ascsagacGfgAfGfCfaacugccauuL96 | 7103 | asAfsugdGc(Agn)guugcuCfcGfucugususg | 7757 | CAACAGACGGAGCAACUGCCAUC | 8009 |
| AD-1708255.1 | ascsggagCfaAfCfUfgccauccgauL96 | 408 | asUfscgdGa(Tgn)ggcaguUfgCfuccguscsu | 7758 | AGACGGAGCAACUGCCAUCCGAG | 8013 |
| AD-1708260.1 | gscsaacuGfcCfAfUfccgaggcucuL96 | 7104 | asGfsagdCc(Tgn)cggaugGfcAfguugcsusc | 7759 | GAGCAACUGCCAUCCGAGGCUCC | 8017 |
| AD-1708342.1 | csasagcaGfaAfCfGfagcucugguuL96 | 415 | asAfsccdAg(Agn)gcucguUfcUfgcuugsgsg | 7760 | CCCAAGCAGAACGAGCUCUGGUG | 8019 |
| AD-1708344.1 | asgscagaAfcGfAfGfcucuggugcuL96 | 7105 | asGfscadCc(Agn)gagcucGfuUfcugcususg | 7761 | CAAGCAGAACGAGCUCUGGUGCU | 8021 |
| AD-1708347.1 | asgsaacgAfgCfUfCfuggugcugguL96 | 7106 | asCfscadGc(Agn)ccagagCfuCfguucusgsc | 7762 | GCAGAACGAGCUCUGGUGCUGGA | 8024 |
| AD-1708350.1 | ascsgagcUfcUfGfGfugcuggagcuL96 | 7107 | asGfscudCc(Agn)gcaccaGfaGfcucgususc | 7763 | GAACGAGCUCUGGUGCUGGAGCU | 8027 |
| AD-1708356.1 | uscsugguGfcUfGfGfagcuagccauL96 | 7108 | asUfsggdCu(Agn)gcuccaGfcAfccagasgsc | 7764 | GCUCUGGUGCUGGAGCUAGCCAA | 8031 |
| AD-1708357.1 | csusggugCfuGfGfAfgcuagccaauL96 | 416 | asUfsugdGc(Tgn)agcuccAfgCfaccagsasg | 7765 | CUCUGGUGCUGGAGCUAGCCAAG | 8032 |
| AD-1708361.1 | usgscuggAfgCfUfAfgccaagcaguL96 | 7109 | asCfsugdCu(Tgn)ggcuagCfuCfcagcascsc | 7766 | GGUGCUGGAGCUAGCCAAGCAGC | 8036 |
| AD-1708365.1 | gsgsagcuAfgCfCfAfagcagcaaauL96 | 7110 | asUfsuudGc(Tgn)gcuuggCfuAfgcuccsasg | 7767 | CUGGAGCUAGCCAAGCAGCAAAU | 8037 |
| AD-1708368.1 | gscsuagcCfaAfGfCfagcaaauccuL96 | 7111 | asGfsgadTu(Tgn)gcugcuUfgGfcuagcsusc | 7768 | GAGCUAGCCAAGCAGCAAAUCCU | 8040 |
| AD-1708369.1 | csusagccAfaGfCfAfgcaaauccuuL96 | 417 | asAfsggdAu(Tgn)ugcugcUfuGfgcuagscsu | 7769 | AGCUAGCCAAGCAGCAAAUCCUG | 8041 |
| AD-1708397.1 | usgsaccaGfuCfGfUfcccagaauauL96 | 7112 | asUfsaudTc(Tgn)gggacgAfcUfggucasgsg | 7770 | UCUGACCAGUCGUCCCAGAAUAA | 8448 |
| AD-1708399.1 | ascscaguCfgUfCfCfcagaauaacuL96 | 7113 | asGfsuudAu(Tgn)cugggaCfgAfcugguscsa | 7771 | UGACCAGUCGUCCCAGAAUAACU | 8050 |
| AD-1708401.1 | csasgucgUfcCfCfAfgaauaacucuL96 | 7114 | asGfsagdTu(Agn)uucuggGfaCfgacugsgsu | 7772 | ACCAGUCGUCCCAGAAUAACUCA | 8052 |
| AD-1708402.1 | asgsucguCfcCfAfGfaauaacucauL96 | 7115 | asUfsgadGu(Tgn)auucugGfgAfcgacusgsg | 7773 | CCAGUCGUCCCAGAAUAACUCAU | 8053 |
| AD-1708403.1 | gsuscgucCfcAfGfAfauaacucauuL96 | 7116 | asAfsugdAg(Tgn)uauucuGfgGfacgacsusg | 7774 | CAGUCGUCCCAGAAUAACUCAUC | 8054 |
| AD-1708405.1 | csgsucccAfgAfAfUfaacucauccuL96 | 7117 | asGfsgadTg(Agn)guuauuCfuGfggacgsasc | 7775 | GUCGUCCCAGAAUAACUCAUCCU | 8056 |
| AD-1708407.1 | uscsccagAfaUfAfAfcucauccucuL96 | 7118 | asGfsagdGa(Tgn)gaguuaUfuCfugggascsg | 7776 | CGUCCCAGAAUAACUCAUCCUCC | 8058 |
| AD-1708408.1 | cscscagaAfuAfAfCfucauccuccuL96 | 7119 | asGfsgadGg(Agn)ugaguuAfuUfcugggsasc | 7777 | GUCCCAGAAUAACUCAUCCUCCA | 8059 |
| AD-1708447.1 | gsgsgaguGfuGfGfCfuccagggaauL96 | 7120 | asUfsucdCc(Tgn)ggagccAfcAfcucccsusg | 7778 | CAGGGAGUGUGGCUCCAGGGAAU | 8062 |
| AD-1708454.1 | gsasggucAfuCfAfGfcuuugcuacuL96 | 7121 | asGfsuadGc(Agn)aagcugAfuGfaccucscsu | 7779 | AGGAGGUCAUCAGCUUUGCUACU | 8069 |
| AD-1708457.1 | gsuscaucAfgCfUfUfugcuacuguuL96 | 7122 | asAfscadGu(Agn)gcaaagCfuGfaugacscsu | 7780 | AGGUCAUCAGCUUUGCUACUGUC | 8072 |
| AD-1708458.1 | uscsaucaGfcUfUfUfgcuacugucuL96 | 7123 | asGfsacdAg(Tgn)agcaaaGfcUfgaugascsc | 7781 | GGUCAUCAGCUUUGCUACUGUCA | 8073 |
| AD-1708462.1 | csasgcuuUfgCfUfAfcugucacaguL96 | 7124 | asCfsugdTg(Agn)caguagCfaAfagcugsasu | 7782 | AUCAGCUUUGCUACUGUCACAGA | 8075 |
| AD-1708464.1 | gscsuuugCfuAfCfUfgucacagacuL96 | 7125 | asGfsucdTg(Tgn)gacaguAfgCfaaagcsusg | 7783 | CAGCUUUGCUACUGUCACAGACU | 8077 |
| AD-1708466.1 | ususugcuAfcUfGfUfcacagacucuL96 | 7126 | asGfsagdTc(Tgn)gugacaGfuAfgcaaasgsc | 7784 | GCUUUGCUACUGUCACAGACUCC | 8079 |
| AD-1708468.1 | usgscuacUfgUfCfAfcagacuccauL96 | 7127 | asUfsggdAg(Tgn)cugugaCfaGfuagcasasa | 7785 | UUUGCUACUGUCACAGACUCCAC | 8081 |
| AD-1708473.1 | csusgucaCfaGfAfCfuccacuucauL96 | 425 | asUfsgadAg(Tgn)ggagucUfgUfgacagsusa | 80 | UACUGUCACAGACUCCACUUCAG | 8083 |
| AD-1708476.1 | uscsacagAfcUfCfCfacuucagccuL96 | 7128 | asGfsgcdTg(Agn)aguggaGfuCfugugascsa | 7786 | UGUCACAGACUCCACUUCAGCCU | 8085 |
| AD-1708478.1 | ascsagacUfcCfAfCfuucagccuauL96 | 7129 | asUfsagdGc(Tgn)gaagugGfaGfucugusgsa | 7787 | UCACAGACUCCACUUCAGCCUAC | 8087 |
| AD-1708482.1 | ascsuccaCfuUfCfAfgccuacagcuL96 | 7130 | asGfscudGu(Agn)ggcugaAfgUfggaguscsu | 7788 | AGACUCCACUUCAGCCUACAGCU | 8091 |
| AD-1708483.1 | csusccacUfuCfAfGfccuacagcuuL96 | 7131 | asAfsgcdTg(Tgn)aggcugAfaGfuggagsusc | 7789 | GACUCCACUUCAGCCUACAGCUC | 8092 |
| AD-1708485.1 | cscsacuuCfaGfCfCfuacagcuccuL96 | 7132 | asGfsgadGc(Tgn)guaggcUfgAfaguggsasg | 7790 | CUCCACUUCAGCCUACAGCUCCC | 8094 |
| AD-1708559.1 | usgsgcacUfcUfUfUfgcuugaggauL96 | 7133 | asUfsccdTc(Agn)agcaaaGfaGfugccasgsg | 7791 | CCUGGCACUCUUUGCUUGAGGAU | 8111 |
| AD-1708561.1 | gscsacucUfuUfGfCfuugaggaucuL96 | 7134 | asGfsaudCc(Tgn)caagcaAfaGfagugcscsa | 7792 | UGGCACUCUUUGCUUGAGGAUCU | 8113 |
| AD-1708564.1 | csuscuuuGfcUfUfGfaggaucuucuL96 | 432 | asGfsaadGa(Tgn)ccucaaGfcAfaagagsusg | 7793 | CACUCUUUGCUUGAGGAUCUUCC | 8115 |
| AD-1708565.1 | uscsuuugCfuUfGfAfggaucuuccuL96 | 7135 | asGfsgadAg(Agn)uccucaAfgCfaaagasgsu | 7794 | ACUCUUUGCUUGAGGAUCUUCCG | 8116 |
| AD-1708567.1 | ususugcuUfgAfGfGfaucuuccgauL96 | 7136 | asUfscgdGa(Agn)gauccuCfaAfgcaaasgsa | 7795 | UCUUUGCUUGAGGAUCUUCCGAU | 8118 |
| AD-1708647.1 | usgsgcugAfgCfAfCfcacaucaccuL96 | 7137 | asGfsgudGa(Tgn)guggugCfuCfagccasgsg | 7796 | UUUGGCUGAGCACCACAUCACCA | 8449 |
| AD-1708648.1 | gsgscugaGfcAfCfCfacaucaccauL96 | 7138 | asUfsggdTg(Agn)ugugguGfcUfcagccsasg | 7797 | UUGGCUGAGCACCACAUCACCAA | 8450 |
| AD-1708650.1 | csusgagcAfcCfAfCfaucaccaacuL96 | 7139 | asGfsuudGg(Tgn)gaugugGfuGfcucagscsc | 7798 | GGCUGAGCACCACAUCACCAACC | 8126 |
| AD-1708657.1 | cscsacauCfaCfCfAfaccugggcuuL96 | 7140 | asAfsgcdCc(Agn)gguuggUfgAfuguggsusg | 7799 | CACCACAUCACCAACCUGGGCUG | 8131 |
| AD-1708662.1 | uscsaccaAfcCfUfGfggcuggcauuL96 | 7141 | asAfsugdCc(Agn)gcccagGfuUfggugasusg | 7800 | CAUCACCAACCUGGGCUGGCAUG | 8451 |
| AD-1708680.1 | cscsuuaaCfuCfUfGfcccucuaguuL96 | 7142 | asAfscudAg(Agn)gggcagAfgUfuaaggsusa | 7801 | UACCUUAACUCUGCCCUCUAGUG | 8138 |
| AD-1708683.1 | usasacucUfgCfCfCfucuaguggcuL96 | 7143 | asGfsccdAc(Tgn)agagggCfaGfaguuasasg | 7802 | CUUAACUCUGCCCUCUAGUGGCU | 8141 |
| AD-1708697.1 | gsuscuggUfgUfCfCfugaaacugcuL96 | 7144 | asGfscadGu(Tgn)ucaggaCfaCfcagacsusu | 7803 | AAGUCUGGUGUCCUGAAACUGCA | 8144 |
| AD-1708698.1 | uscsugguGfuCfCfUfgaaacugcauL96 | 7145 | asUfsgcdAg(Tgn)uucaggAfcAfccagascsu | 7804 | AGUCUGGUGUCCUGAAACUGCAA | 8145 |
| AD-1708700.1 | usgsguguCfcUfGfAfaacugcaacuL96 | 7146 | asGfsuudGc(Agn)guuucaGfgAfcaccasgsa | 7805 | UCUGGUGUCCUGAAACUGCAACU | 8147 |
| AD-1708704.1 | gsusccugAfaAfCfUfgcaacuagauL96 | 439 | asUfscudAg(Tgn)ugcaguUfuCfaggacsasc | 7806 | GUGUCCUGAAACUGCAACUAGAC | 8151 |
| AD-1708706.1 | cscsugaaAfcUfGfCfaacuagacuuL96 | 7147 | asAfsgudCu(Agn)guugcaGfuUfucaggsasc | 7807 | GUCCUGAAACUGCAACUAGACUG | 8153 |
| AD-1708707.1 | csusgaaaCfuGfCfAfacuagacuguL96 | 7148 | asCfsagdTc(Tgn)aguugcAfgUfuucagsgsa | 7808 | UCCUGAAACUGCAACUAGACUGC | 8154 |
| AD-1708709.1 | gsasaacuGfcAfAfCfuagacugcauL96 | 7149 | asUfsgcdAg(Tgn)cuaguuGfcAfguuucsasg | 7809 | CUGAAACUGCAACUAGACUGCAG | 8156 |
| AD-1708711.1 | asascugcAfaCfUfAfgacugcagauL96 | 7150 | asUfscudGc(Agn)gucuagUfuGfcaguususc | 7810 | GAAACUGCAACUAGACUGCAGAC | 8158 |
| AD-1708719.1 | gscsaacaGfcAfCfAfguuacuggauL96 | 7151 | asUfsccdAg(Tgn)aacuguGfcUfguugcscsu | 7811 | AGGCAACAGCACAGUUACUGGAC | 8161 |
| AD-1708721.1 | asascagcAfcAfGfUfuacuggacauL96 | 7152 | asUfsgudCc(Agn)guaacuGfuGfcuguusgsc | 7812 | ACAACAGCACAGUUACUGGACAA | 8452 |
| AD-1708724.1 | asgscacaGfuUfAfCfuggacaaccuL96 | 7153 | asGfsgudTg(Tgn)ccaguaAfcUfgugcusgsu | 7813 | ACAGCACAGUUACUGGACAACCG | 8166 |
| AD-1708746.1 | ususggacAfcAfGfCfaggacaccauL96 | 7154 | asUfsggdTg(Tgn)ccugcuGfuGfuccaasgsa | 7814 | UCUUGGACACAGCAGGACACCAG | 8168 |
| AD-1708748.1 | gsgsacacAfgCfAfGfgacaccagcuL96 | 7155 | asGfscudGg(Tgn)guccugCfuGfuguccsasa | 7815 | CUGGACACAGCAGGACACCAGCA | 8453 |
| AD-1708751.1 | csascagcAfgGfAfCfaccagcagcuL96 | 7156 | asGfscudGc(Tgn)ggugucCfuGfcugugsusc | 7816 | GACACAGCAGGACACCAGCAGCC | 8173 |
| AD-1708759.1 | gsascaccAfgCfAfGfcccuuccuauL96 | 443 | asUfsagdGa(Agn)gggcugCfuGfgugucscsu | 7817 | AGGACACCAGCAGCCCUUCCUAG | 8176 |
| AD-1708763.1 | cscsagcaGfcCfCfUfuccuagagcuL96 | 7157 | asGfscudCu(Agn)ggaaggGfcUfgcuggsusg | 7818 | CACCAGCAGCCCUUCCUAGAGCU | 8179 |
| AD-1708764.1 | csasgcagCfcCfUfUfccuagagcuuL96 | 7158 | asAfsgcdTc(Tgn)aggaagGfgCfugcugsgsu | 7819 | ACCAGCAGCCCUUCCUAGAGCUU | 8180 |
| AD-1708766.1 | gscsagccCfuUfCfCfuagagcuuauL96 | 7159 | asUfsaadGc(Tgn)cuaggaAfgGfgcugcsusg | 7820 | CAGCAGCCCUUCCUAGAGCUUAA | 8181 |
| AD-1708771.1 | cscsuuccUfaGfAfGfcuuaagaucuL96 | 444 | asGfsaudCu(Tgn)aagcucUfaGfgaaggsgsc | 7821 | GCCCUUCCUAGAGCUUAAGAUCC | 8186 |
| AD-1708813.1 | ususacguAfgAfCfUfuccaggaacuL96 | 7160 | asGfsuudCc(Tgn)ggaaguCfuAfcguaasusg | 7822 | CAUUACGUAGACUUCCAGGAACU | 8187 |
| AD-1708819.1 | asgsacuuCfcAfGfGfaacugggauuL96 | 7161 | asAfsucdCc(Agn)guuccuGfgAfagucusasc | 7823 | GUAGACUUCCAGGAACUGGGAUG | 8192 |
| AD-1708842.1 | gsusaccaGfcUfGfAfauuacugcauL96 | 7162 | asUfsgcdAg(Tgn)aauucaGfcUfgguacscsc | 7824 | GGGUACCAGCUGAAUUACUGCAG | 8199 |
| AD-1708844.1 | ascscagcUfgAfAfUfuacugcaguuL96 | 7163 | asAfscudGc(Agn)guaauuCfaGfcuggusasc | 7825 | GUACCAGCUGAAUUACUGCAGUG | 8201 |
| AD-1708849.1 | csusgaauUfaCfUfGfcagugggcauL96 | 7164 | asUfsgcdCc(Agn)cugcagUfaAfuucagscsu | 7826 | AGCUGAAUUACUGCAGUGGGCAG | 8206 |
| AD-1708856.1 | ascsugcaGfuGfGfGfcagugcccuuL96 | 452 | asAfsggdGc(Agn)cugcccAfcUfgcagusasa | 7827 | UUACUGCAGUGGGCAGUGCCCUC | 8209 |
| AD-1708872.1 | csasggcaUfuGfCfUfgccucuuucuL96 | 453 | asGfsaadAg(Agn)ggcagcAfaUfgccugsgsg | 7828 | CCCAGGCAUUGCUGCCUCUUUCC | 8210 |
| AD-1708875.1 | csasuugcUfgCfCfUfcuuuccauuuL96 | 7165 | asAfsaudGg(Agn)aagaggCfaGfcaaugscsc | 7829 | GGCAUUGCUGCCUCUUUCCAUUC | 8214 |
| AD-1708882.1 | gscscucuUfuCfCfAfuucugccguuL96 | 7166 | asAfscgdGc(Agn)gaauggAfaAfgaggcsasg | 7830 | CUGCCUCUUUCCAUUCUGCCGUC | 8221 |
| AD-1708887.1 | ususuccaUfuCfUfGfccgucuucauL96 | 454 | asUfsgadAg(Agn)cggcagAfaUfggaaasgsa | 7831 | UCUUUCCAUUCUGCCGUCUUCAG | 8226 |
| AD-1708889.1 | uscscauuCfuGfCfCfgucuucagcuL96 | 7167 | asGfscudGa(Agn)gacggcAfgAfauggasasa | 7832 | UUUCCAUUCUGCCGUCUUCAGCC | 8228 |
| AD-1708890.1 | cscsauucUfgCfCfGfucuucagccuL96 | 7168 | asGfsgcdTg(Agn)agacggCfaGfaauggsasa | 7833 | UUCCAUUCUGCCGUCUUCAGCCU | 8229 |
| AD-1708899.1 | csgsucuuCfaGfCfCfuccucaaaguL96 | 455 | asCfsuudTg(Agn)ggaggcUfgAfagacgsgsc | 7834 | GCCGUCUUCAGCCUCCUCAAAGC | 8233 |
| AD-1708901.1 | uscsuucaGfcCfUfCfcucaaagccuL96 | 7169 | asGfsgcdTu(Tgn)gaggagGfcUfgaagascsg | 7835 | CGUCUUCAGCCUCCUCAAAGCCA | 8235 |
| AD-1708902.1 | csusucagCfcUfCfCfucaaagccauL96 | 7170 | asUfsggdCu(Tgn)ugaggaGfgCfugaagsasc | 7836 | GUCUUCAGCCUCCUCAAAGCCAA | 8236 |
| AD-1708907.1 | gscscuccUfcAfAfAfgccaacaauuL96 | 7171 | asAfsuudGu(Tgn)ggcuuuGfaGfgaggcsusg | 7837 | CAGCCUCCUCAAAGCCAACAAUC | 8240 |
| AD-1708908.1 | cscsuccuCfaAfAfGfccaacaaucuL96 | 7172 | asGfsaudTg(Tgn)uggcuuUfgAfggaggscsu | 7838 | AGCCUCCUCAAAGCCAACAAUCC | 8241 |
| AD-1708911.1 | cscsucaaAfgCfCfAfacaauccuuuL96 | 456 | asAfsagdGa(Tgn)uguuggCfuUfugaggsasg | 7839 | CUCCUCAAAGCCAACAAUCCUUG | 8244 |
| AD-1708921.1 | asascaauCfcUfUfGfgccugccaguL96 | 7173 | asCfsugdGc(Agn)ggccaaGfgAfuuguusgsg | 7840 | CCAACAAUCCUUGGCCUGCCAGU | 8250 |
| AD-1708956.1 | uscsuccuCfuAfCfCfuggaucauauL96 | 7174 | asUfsaudGa(Tgn)ccagguAfgAfggagasgsa | 7841 | UCUCUCCUCUACCUGGAUCAUAA | 8262 |
| AD-1708957.1 | csusccucUfaCfCfUfggaucauaauL96 | 7175 | asUfsuadTg(Agn)uccaggUfaGfaggagsasg | 7842 | CUCUCCUCUACCUGGAUCAUAAU | 8263 |
| AD-1708962.1 | csusaccuGfgAfUfCfauaauggcauL96 | 7176 | asUfsgcdCa(Tgn)uaugauCfcAfgguagsasg | 7843 | CUCUACCUGGAUCAUAAUGGCAA | 8268 |
| AD-1708963.1 | usasccugGfaUfCfAfuaauggcaauL96 | 461 | asUfsugdCc(Agn)uuaugaUfcCfagguasgsa | 7844 | UCUACCUGGAUCAUAAUGGCAAU | 8269 |
| AD-1708971.1 | uscsauaaUfgGfCfAfauguggucauL96 | 7177 | asUfsgadCc(Agn)cauugcCfaUfuaugasusc | 7845 | GAUCAUAAUGGCAAUGUGGUCAA | 8277 |
| AD-1708974.1 | usasauggCfaAfUfGfuggucaagauL96 | 7178 | asUfscudTg(Agn)ccacauUfgCfcauuasusg | 7846 | CAUAAUGGCAAUGUGGUCAAGAC | 8280 |
| AD-1708976.1 | asusggcaAfuGfUfGfgucaagacguL96 | 7179 | asCfsgudCu(Tgn)gaccacAfuUfgccaususa | 7847 | UAAUGGCAAUGUGGUCAAGACGG | 8282 |
| AD-1708977.1 | usgsgcaaUfgUfGfGfucaagacgguL96 | 7180 | asCfscgdTc(Tgn)ugaccaCfaUfugccasusu | 7848 | AAUGGCAAUGUGGUCAAGACGGA | 8283 |
| AD-1708984.1 | gsusggucAfaGfAfCfggaugugccuL96 | 7181 | asGfsgcdAc(Agn)uccgucUfuGfaccacsasu | 7849 | AUGUGGUCAAGACGGAUGUGCCA | 8290 |
| AD-1708986.1 | gsgsucaaGfaCfGfGfaugugccaguL96 | 7182 | asCfsugdGc(Agn)cauccgUfcUfugaccsasc | 7850 | GUGGUCAAGACGGAUGUGCCAGA | 8292 |
| AD-1708990.1 | asasgacgGfaUfGfUfgccagauauuL96 | 463 | asAfsuadTc(Tgn)ggcacaUfcCfgucuusgsa | 7851 | UCAAGACGGAUGUGCCAGAUAUG | 8296 |
| AD-1709009.1 | usgsguggUfgGfAfGfgccuguggcuL96 | 7183 | asGfsccdAc(Agn)ggccucCfaCfcaccasusa | 7852 | UAUGGUGGUGGAGGCCUGUGGCU | 8307 |
| AD-1709021.1 | cscsugugGfcUfGfCfagcuagcaauL96 | 7184 | asUfsugdCu(Agn)gcugcaGfcCfacaggscsc | 7853 | GGCCUGUGGCUGCAGCUAGCAAG | 8311 |
| AD-1709022.1 | csusguggCfuGfCfAfgcuagcaaguL96 | 7185 | asCfsuudGc(Tgn)agcugcAfgCfcacagsgsc | 7854 | GCCUGUGGCUGCAGCUAGCAAGA | 8312 |
| AD-1709026.1 | gsgscugcAfgCfUfAfgcaagaggauL96 | 7186 | asUfsccdTc(Tgn)ugcuagCfuGfcagccsasc | 7855 | GUGGCUGCAGCUAGCAAGAGGAC | 8316 |
| AD-1709028.1 | csusgcagCfuAfGfCfaagaggaccuL96 | 7187 | asGfsgudCc(Tgn)cuugcuAfgCfugcagscsc | 7856 | GGCUGCAGCUAGCAAGAGGACCU | 8318 |
| AD-1709031.1 | csasgcuaGfcAfAfGfaggaccugguL96 | 7188 | asCfscadGg(Tgn)ccucuuGfcUfagcugscsa | 7857 | UGCAGCUAGCAAGAGGACCUGGG | 8321 |
| AD-1709032.1 | gscsuuugGfaGfUfGfaagagaccauL96 | 7189 | asUfsggdTc(Tgn)cuucacUfcCfaaagcscsc | 7858 | GGGCUUUGGAGUGAAGAGACCAA | 8322 |
| AD-1709034.1 | ususuggaGfuGfAfAfgagaccaaguL96 | 7190 | asCfsuudGg(Tgn)cucuucAfcUfccaaasgsc | 7859 | GCUUUGGAGUGAAGAGACCAAGA | 8324 |
| AD-1709038.1 | gsasgugaAfgAfGfAfccaagaugauL96 | 7191 | asUfscadTc(Tgn)uggucuCfuUfcacucscsa | 7860 | UGGAGUGAAGAGACCAAGAUGAA | 8327 |
| AD-1709041.1 | usgsaagaGfaCfCfAfagaugaaguuL96 | 7192 | asAfscudTc(Agn)ucuuggUfcUfcuucascsu | 7861 | AGUGAAGAGACCAAGAUGAAGUU | 8328 |
| AD-1709044.1 | asgsagacCfaAfGfAfugaaguuucuL96 | 468 | asGfsaadAc(Tgn)ucaucuUfgGfucucususc | 7862 | GAAGAGACCAAGAUGAAGUUUCC | 8330 |
| AD-1709052.1 | asgsaugaAfgUfUfUfcccaggcacuL96 | 7193 | asGfsugdCc(Tgn)gggaaaCfuUfcaucususg | 7863 | CAAGAUGAAGUUUCCCAGGCACA | 8338 |
| AD-1709056.1 | gsasaguuUfcCfCfAfggcacaggguL96 | 7194 | asCfsccdTg(Tgn)gccuggGfaAfacuucsasu | 7864 | AUGAAGUUUCCCAGGCACAGGGC | 8341 |
| AD-1709090.1 | ascsccaaCfaAfCfCfaccuggcaauL96 | 471 | asUfsugdCc(Agn)ggugguUfgUfugggususg | 7865 | CAACCCAACAACCACCUGGCAAU | 8349 |
| AD-1709097.1 | asasccacCfuGfGfCfaauaugacuuL96 | 7195 | asAfsgudCa(Tgn)auugccAfgGfugguusgsu | 7866 | ACAACCACCUGGCAAUAUGACUC | 8356 |
| AD-1709098.1 | ascscaccUfgGfCfAfauaugacucuL96 | 7196 | asGfsagdTc(Agn)uauugcCfaGfguggususg | 7867 | CAACCACCUGGCAAUAUGACUCA | 8357 |
| AD-1709100.1 | csasccugGfcAfAfUfaugacucacuL96 | 7197 | asGfsugdAg(Tgn)cauauuGfcCfaggugsgsu | 7868 | ACCACCUGGCAAUAUGACUCACU | 8359 |
| AD-1709102.1 | cscsuggcAfaUfAfUfgacucacuuuL96 | 7198 | asAfsagdTg(Agn)gucauaUfuGfccaggsusg | 7869 | CACCUGGCAAUAUGACUCACUUG | 8361 |
| AD-1709104.1 | usgsgcaaUfaUfGfAfcucacuugauL96 | 7199 | asUfscadAg(Tgn)gagucaUfaUfugccasgsg | 7870 | CCUGGCAAUAUGACUCACUUGAC | 8363 |
| AD-1709106.1 | gscsaauaUfgAfCfUfcacuugaccuL96 | 7200 | asGfsgudCa(Agn)gugaguCfaUfauugcscsa | 7871 | UGGCAAUAUGACUCACUUGACCC | 8365 |
| AD-1709111.1 | gsgsacccAfaAfUfGfggcacuuucuL96 | 7201 | asGfsaadAg(Tgn)gcccauUfuGfgguccscsa | 7872 | UGGGACCCAAAUGGGCACUUUCU | 8366 |
| AD-1709120.1 | usgsggcaCfuUfUfCfuugucugaguL96 | 7202 | asCfsucdAg(Agn)caagaaAfgUfgcccasusu | 7873 | AAUGGGCACUUUCUUGUCUGAGA | 8374 |
| AD-1709122.1 | gsgscacuUfuCfUfUfgucugagacuL96 | 7203 | asGfsucdTc(Agn)gacaagAfaAfgugccscsa | 7874 | UGGGCACUUUCUUGUCUGAGACU | 8376 |
| AD-1709124.1 | csascuuuCfuUfGfUfcugagacucuL96 | 7204 | asGfsagdTc(Tgn)cagacaAfgAfaagugscsc | 7875 | GGCACUUUCUUGUCUGAGACUCU | 8378 |
| AD-1709126.1 | csusuucuUfgUfCfUfgagacucuguL96 | 7205 | asCfsagdAg(Tgn)cucagaCfaAfgaaagsusg | 7876 | CACUUUCUUGUCUGAGACUCUGG | 8380 |
| AD-1709128.1 | ususcuugUfcUfGfAfgacucuggcuL96 | 7206 | asGfsccdAg(Agn)gucucaGfaCfaagaasasg | 7877 | CUUUCUUGUCUGAGACUCUGGCU | 8382 |
| AD-1709130.1 | csusugucUfgAfGfAfcucuggcuuuL96 | 7207 | asAfsagdCc(Agn)gagucuCfaGfacaagsasa | 7878 | UUCUUGUCUGAGACUCUGGCUUG | 8454 |
| AD-1709148.1 | usasuuccAfgGfUfUfggcugauguuL96 | 7208 | asAfscadTc(Agn)gccaacCfuGfgaauasasg | 7879 | CUUAUUCCAGGUUGGCUGAUGUG | 8385 |
| AD-1709151.1 | uscscaggUfuGfGfCfugauguguuuL96 | 476 | asAfsacdAc(Agn)ucagccAfaCfcuggasasu | 7880 | AUUCCAGGUUGGCUGAUGUGUUG | 8388 |
| AD-1709201.1 | csuscucuCfcUfCfUfaccuggaucuL96 | 7209 | asGfsaudCc(Agn)gguagaGfgAfgagagsasg | 7881 | CUCUCUCUCCUCUACCUGGAUCA | 8259 |
| AD-1709237.1 | gsasuuucCfuGfCfCfcuaaguccuuL96 | 480 | asAfsggdAc(Tgn)uagggcAfgGfaaaucsasu | 7882 | AUGAUUUCCUGCCCUAAGUCCUG | 8391 |
| AD-1709239.1 | ususuccuGfcCfCfUfaaguccuguuL96 | 7210 | asAfscadGg(Agn)cuuaggGfcAfggaaasusc | 7883 | GAUUUCCUGCCCUAAGUCCUGUG | 8392 |
| AD-1709253.1 | asgsaagaUfgUfCfAfgggacuagguL96 | 7211 | asCfscudAg(Tgn)cccugaCfaUfcuucuscsa | 7884 | UGAGAAGAUGUCAGGGACUAGGG | 8393 |
| AD-1709254.1 | asasgaugUfcAfGfGfgacuagggauL96 | 7212 | asUfsccdCu(Agn)gucccuGfaCfaucuuscsu | 7885 | AGAAGAUGUCAGGGACUAGGGAG | 8395 |
| AD-1709298.1 | asgsgaggAfaGfCfAfgauagaugguL96 | 7213 | asCfscadTc(Tgn)aucugcUfuCfcuccuscsc | 7886 | GGAGGAGGAAGCAGAUAGAUGGU | 8400 |
| AD-1709300.1 | gsasggaaGfcAfGfAfuagauggucuL96 | 7214 | asGfsacdCa(Tgn)cuaucuGfcUfuccucscsu | 7887 | AGGAGGAAGCAGAUAGAUGGUCC | 8402 |
| AD-1709301.1 | asgsgaagCfaGfAfUfagaugguccuL96 | 7215 | asGfsgadCc(Agn)ucuaucUfgCfuuccuscsc | 7888 | GGAGGAAGCAGAUAGAUGGUCCA | 8403 |
| AD-1709304.1 | asasgcagAfuAfGfAfugguccagcuL96 | 7216 | asGfscudGg(Agn)ccaucuAfuCfugcuuscsc | 7889 | GGAAGCAGAUAGAUGGUCCAGCA | 8406 |
| AD-1709307.1 | csasgauaGfaUfGfGfuccagcagguL96 | 7217 | asCfscudGc(Tgn)ggaccaUfcUfaucugscsu | 7890 | AGCAGAUAGAUGGUCCAGCAGGC | 8409 |
| AD-1709310.1 | asusagauGfgUfCfCfagcaggcuuuL96 | 7218 | asAfsagdCc(Tgn)gcuggaCfcAfucuauscsu | 7891 | AGAUAGAUGGUCCAGCAGGCUUG | 8412 |
| AD-1709314.1 | asusggucCfaGfCfAfggcuugaaguL96 | 7219 | asCfsuudCa(Agn)gccugcUfgGfaccauscsu | 7892 | AGAUGGUCCAGCAGGCUUGAAGC | 8416 |
| AD-1709315.1 | usgsguccAfgCfAfGfgcuugaagcuL96 | 7220 | asGfscudTc(Agn)agccugCfuGfgaccasusc | 7893 | GAUGGUCCAGCAGGCUUGAAGCA | 8417 |
| AD-1709317.1 | gsusccagCfaGfGfCfuugaagcaguL96 | 7221 | asCfsugdCu(Tgn)caagccUfgCfuggacscsa | 7894 | UGGUCCAGCAGGCUUGAAGCAGG | 8419 |
| AD-1709372.1 | gsgsucaaGfaGfGfGfagaugggcauL96 | 7222 | asUfsgcdCc(Agn)ucucccUfcUfugaccsusu | 7895 | AGGGUCAAGAGGGAGAUGGGCAA | 8455 |
| AD-1709390.1 | gscsgcugAfgGfGfAfggaugcuuauL96 | 7223 | asUfsaadGc(Agn)uccuccCfuCfagcgcscsu | 7896 | AGGCGCUGAGGGAGGAUGCUUAG | 8425 |
| AD-1709410.1 | gscsacuaAfgCfCfUfaagaaguucuL96 | 7224 | asGfsaadCu(Tgn)cuuaggCfuUfagugcscsu | 7897 | AGGCACUAAGCCUAAGAAGUUCC | 8427 |
| AD-1709411.1 | csascuaaGfcCfUfAfagaaguuccuL96 | 7225 | asGfsgadAc(Tgn)ucuuagGfcUfuagugscsc | 7898 | GGCACUAAGCCUAAGAAGUUCCC | 8428 |
| AD-1709429.1 | csasggacCfcAfCfUfgggagacaauL96 | 7226 | asUfsugdTc(Tgn)cccaguGfgGfuccugsusc | 7899 | GACAGGACCCACUGGGAGACAAG | 8430 |
| AD-1709431.1 | gsgsacccAfcUfGfGfgagacaagcuL96 | 7227 | asGfscudTg(Tgn)cucccaGfuGfgguccsusg | 7900 | CAGGACCCACUGGGAGACAAGCA | 8432 |
| AD-1709433.1 | ascsccacUfgGfGfAfgacaagcauuL96 | 7228 | asAfsugdCu(Tgn)gucuccCfaGfuggguscsc | 7901 | GGACCCACUGGGAGACAAGCAUU | 8434 |
| AD-1709434.1 | cscscacuGfgGfAfGfacaagcauuuL96 | 7229 | asAfsaudGc(Tgn)ugucucCfcAfgugggsusc | 7902 | GACCCACUGGGAGACAAGCAUUU | 8435 |
| AD-1709442.1 | gsasgacaAfgCfAfUfuuauacuuuuL96 | 7230 | asAfsaadGu(Agn)uaaaugCfuUfgucucscsc | 7903 | GGGAGACAAGCAUUUAUACUUUC | 8442 |
| AD-1709443.1 | asgsacaaGfcAfUfUfuauacuuucuL96 | 7231 | asGfsaadAg(Tgn)auaaauGfcUfugucuscsc | 7904 | GGAGACAAGCAUUUAUACUUUCU | 8443 |
| AD-1709502.1 | asgsgucaAfgAfGfGfgagaugggcuL96 | 7232 | asGfsccdCa(Tgn)cucccuCfuUfgaccususc | 7905 | GAAGGUCAAGAGGGAGAUGGGCA | 8421 |
| AD-1711741.1 | csasagcacaGfCfUfauccaucaguL96 | 6591 | asdCsugdAudGgauadGcUfgugcuugscsu | 7906 | GUCAAGCACAGCUAUCCAUCAGA | 7962 |
| AD-1711742.1 | asuscuacuuUfCfAfgccuuccuguL96 | 6613 | asdCsagdGadAggcudGaAfaguagauscsu | 7907 | UGAUCUACUUUCAGCCUUCCUGA | 7984 |
| AD-1711743.1 | asgsccaagcAfGfCfaaauccugguL96 | 6672 | asdCscadGgdAuuugdCuGfcuuggcuscsu | 7908 | CUAGCCAAGCAGCAAAUCCUGGA | 8043 |
| AD-1711744.1 | ascscagucgUfCfCfcagaauaacuL96 | 6679 | asdGsuudAudTcuggdGaCfgacugguscsu | 76 | UGACCAGUCGUCCCAGAAUAACU | 8050 |
| AD-1711745.1 | cscsuggcacUfCfUfuugcuugaguL96 | 6738 | asdCsucdAadGcaaadGaGfugccaggscsu | 7909 | UUCCUGGCACUCUUUGCUUGAGG | 8109 |
| AD-1711746.1 | csusggcacuCfUfUfugcuugagguL96 | 6739 | asdCscudCadAgcaadAgAfgugccagscsu | 7910 | UCCUGGCACUCUUUGCUUGAGGA | 8110 |
| AD-1711747.1 | csusuugcuuGfAfGfgaucuuccguL96 | 6746 | asdCsggdAadGauccdTcAfagcaaagscsu | 7911 | CUCUUUGCUUGAGGAUCUUCCGA | 8117 |
| AD-1711748.1 | ususugcuugAfGfGfaucuuccgauL96 | 6747 | asdTscgdGadAgaucdCuCfaagcaaascsu | 7912 | UCUUUGCUUGAGGAUCUUCCGAU | 8118 |
| AD-1711749.1 | ususgcuugaGfGfAfucuuccgauuL96 | 6748 | asdAsucdGgdAagaudCcUfcaagcaascsu | 7913 | CUUUGCUUGAGGAUCUUCCGAUG | 8119 |
| AD-1711750.1 | usgscuugagGfAfUfcuuccgauguL96 | 6749 | asdCsaudCgdGaagadTcCfucaagcascsu | 7914 | UUUGCUUGAGGAUCUUCCGAUGG | 8120 |
| AD-1711751.1 | gscsuugaggAfUfCfuuccgaugguL96 | 6750 | asdCscadTcdGgaagdAuCfcucaagcscsu | 7915 | UUGCUUGAGGAUCUUCCGAUGGG | 8121 |
| AD-1711752.1 | asgscaccacAfUfCfaccaaccuguL96 | 6758 | asdCsagdGudTggugdAuGfuggugcuscsu | 7916 | UGAGCACCACAUCACCAACCUGG | 8129 |
| AD-1711753.1 | ascsguagacUfUfCfcaggaacuguL96 | 6818 | asdCsagdTudCcuggdAaGfucuacguscsu | 7917 | UUACGUAGACUUCCAGGAACUGG | 8189 |
| AD-1711754.1 | csgsuagacuUfCfCfaggaacugguL96 | 6819 | asdCscadGudTccugdGaAfgucuacgscsu | 7918 | UACGUAGACUUCCAGGAACUGGG | 8190 |
| AD-1711755.1 | gsascuuccaGfGfAfacugggauguL96 | 6822 | asdCsaudCcdCaguudCcUfggaagucscsu | 7919 | UAGACUUCCAGGAACUGGGAUGG | 8193 |
| AD-1711756.1 | usgscugccuCfUfUfuccauucuguL96 | 6846 | asdCsagdAadTggaadAgAfggcagcascsu | 7920 | AUUGCUGCCUCUUUCCAUUCUGC | 8217 |
| AD-1711757.1 | gscsugccucUfUfUfccauucugcuL96 | 6847 | asdGscadGadAuggadAaGfaggcagcscsu | 7921 | UUGCUGCCUCUUUCCAUUCUGCC | 8218 |
| AD-1711758.1 | csusgccucuUfUfCfcauucugccuL96 | 6848 | asdGsgcdAgdAauggdAaAfgaggcagscsu | 7922 | UGCUGCCUCUUUCCAUUCUGCCG | 8219 |
| AD-1711759.1 | ususccauucUfGfCfcgucuucaguL96 | 6856 | asdCsugdAadGacggdCaGfaauggaascsu | 7923 | CUUUCCAUUCUGCCGUCUUCAGC | 8227 |
| AD-1711760.1 | cscsucaaagCfCfAfacaauccuuuL96 | 6873 | asdAsagdGadTuguudGgCfuuugaggscsu | 7924 | CUCCUCAAAGCCAACAAUCCUUG | 8244 |
| AD-1711761.1 | csuscaaagcCfAfAfcaauccuuguL96 | 6874 | asdCsaadGgdAuugudTgGfcuuugagscsu | 7925 | UCCUCAAAGCCAACAAUCCUUGG | 8245 |
| AD-1711762.1 | usgsugucccUfAfCfugcccgaaguL96 | 6884 | asdCsuudCgdGgcagdTaGfggacacascsu | 7926 | GUUGUGUCCCUACUGCCCGAAGG | 8255 |
| AD-1711763.1 | gsusgucccuAfCfUfgcccgaagguL96 | 6885 | asdCscudTcdGggcadGuAfgggacacscsu | 7927 | UUGUGUCCCUACUGCCCGAAGGC | 8256 |
| AD-1711764.1 | cscsucuaccUfGfGfaucauaauguL96 | 6894 | asdCsaudTadTgaucdCaGfguagaggscsu | 7928 | CUCCUCUACCUGGAUCAUAAUGG | 8265 |
| AD-1711765.1 | csuscuaccuGfGfAfucauaaugguL96 | 6895 | asdCscadTudAugaudCcAfgguagagscsu | 7929 | UCCUCUACCUGGAUCAUAAUGGC | 8266 |
| AD-1711766.1 | gsgsaucauaAfUfGfgcaaugugguL96 | 6903 | asdCscadCadTugccdAuUfaugauccscsu | 7930 | CUGGAUCAUAAUGGCAAUGUGGU | 8274 |
| AD-1711767.1 | gsasucauaaUfGfGfcaaugugguuL96 | 6904 | asdAsccdAcdAuugcdCaUfuaugaucscsu | 7931 | UGGAUCAUAAUGGCAAUGUGGUC | 8275 |
| AD-1711768.1 | asusggcaauGfUfGfgucaagacguL96 | 6911 | asdCsgudCudTgaccdAcAfuugccauscsu | 7932 | UAAUGGCAAUGUGGUCAAGACGG | 8282 |
| AD-1711769.1 | gsgscaauguGfGfUfcaagacggauL96 | 6913 | asdTsccdGudCuugadCcAfcauugccscsu | 7933 | AUGGCAAUGUGGUCAAGACGGAU | 8284 |
| AD-1711770.1 | gscsaaugugGfUfCfaagacggauuL96 | 6914 | asdAsucdCgdTcuugdAcCfacauugcscsu | 7934 | UGGCAAUGUGGUCAAGACGGAUG | 8285 |
| AD-1711771.1 | usgsccagauAfUfGfgugguggaguL96 | 6934 | asdCsucdCadCcaccdAuAfucuggcascsu | 7935 | UGUGCCAGAUAUGGUGGUGGAGG | 8305 |
| AD-1711772.1 | usgsgcugcaGfCfUfagcaagagguL96 | 6944 | asdCscudCudTgcuadGcUfgcagccascsu | 7936 | UGUGGCUGCAGCUAGCAAGAGGA | 8315 |
| AD-1711773.1 | gsgsagugaaGfAfGfaccaagauguL96 | 6955 | asdCsaudCudTggucdTcUfucacuccscsu | 7937 | UUGGAGUGAAGAGACCAAGAUGA | 8326 |
| AD-1711774.1 | asasgagaccAfAfGfaugaaguuuuL96 | 6958 | asdAsaadCudTcaucdTuGfgucucuuscsu | 7938 | UGAAGAGACCAAGAUGAAGUUUC | 8329 |
| AD-1711775.1 | ususccagguUfGfGfcugauguguuL96 | 7016 | asdAscadCadTcagcdCaAfccuggaascsu | 7939 | UAUUCCAGGUUGGCUGAUGUGUU | 8387 |
| AD-1711776.1 | cscsagguugGfCfUfgauguguuguL96 | 7018 | asdCsaadCadCaucadGcCfaaccuggscsu | 7940 | UUCCAGGUUGGCUGAUGUGUUGG | 8389 |
| AD-1711777.1 | csasgguuggCfUfGfauguguugguL96 | 7019 | asdCscadAcdAcaucdAgCfcaaccugscsu | 7941 | UCCAGGUUGGCUGAUGUGUUGGG | 8390 |
| AD-1711778.1 | gsusccagcaGfGfCfuugaagcaguL96 | 7048 | asdCsugdCudTcaagdCcUfgcuggacscsu | 7942 | UGGUCCAGCAGGCUUGAAGCAGG | 8419 |
| AD-1711779.1 | gsgsagacaaGfCfAfuuuauacuuuL96 | 7070 | asdAsagdTadTaaaudGcUfugucuccscsu | 7943 | UGGGAGACAAGCAUUUAUACUUU | 8441 |

TABLE 21A
Single dose screen for dsRNA agents targeting INHBE in Hep3b cells

| | 10 nM | | 1 nM | | 0.1 nM | |
|---|---|---|---|---|---|---|
| | % Avg | | % Avg | | % Avg | |
| Duplex | message | | message | | message | |
| Name | remaining | STDEV | remaining | STDEV | remaining | STDEV |
| AD-1708158.1 | 34.54 | 8.05 | 71.33 | 9.23 | 107.64 | 15.41 |
| AD-1706305.1 | 35.48 | 5.41 | 73.21 | 3.56 | 104.59 | 20.16 |
| AD-1706299.1 | 36.87 | 8.53 | 79.64 | 15.65 | 114.93 | 19.80 |
| AD-1706300.1 | 37.80 | 4.13 | 73.84 | 6.08 | 114.88 | 21.29 |
| AD-1706297.1 | 39.37 | 5.42 | 70.23 | 10.61 | 86.59 | 21.82 |
| AD-1706306.1 | 39.71 | 4.79 | 72.04 | 14.92 | 130.55 | 3.74 |
| AD-1706410.1 | 40.85 | 1.71 | 62.77 | 3.66 | 109.95 | 30.95 |
| AD-1706298.1 | 40.98 | 10.04 | 64.11 | 16.28 | 101.43 | 28.55 |
| AD-1706304.1 | 44.69 | 8.85 | 92.06 | 13.52 | 109.73 | 9.34 |
| AD-1706302.1 | 50.26 | 9.48 | 84.61 | 11.73 | 122.60 | 29.73 |
| AD-1706292.1 | 52.60 | 17.55 | 75.07 | 12.50 | 124.87 | 12.45 |
| AD-1706296.1 | 52.71 | 5.68 | 75.22 | 5.52 | 131.62 | 16.26 |
| AD-1708152.1 | 53.50 | 0.47 | 87.86 | 10.25 | 136.07 | 12.18 |
| AD-1706307.1 | 54.31 | 26.05 | 89.68 | 30.45 | 110.24 | 8.38 |
| AD-1706301.1 | 54.75 | 12.47 | 84.13 | 5.13 | 124.19 | 16.94 |
| AD-1706287.1 | 55.23 | 11.82 | 83.57 | 17.26 | 115.00 | 21.38 |
| AD-1706411.1 | 55.84 | 10.32 | 89.82 | 7.68 | 103.89 | 21.25 |
| AD-1706295.1 | 56.58 | 15.21 | 71.06 | 10.26 | 113.27 | 21.71 |
| AD-1706294.1 | 56.72 | 15.26 | 76.70 | 11.02 | 129.67 | 23.61 |
| AD-1706409.1 | 56.75 | 9.70 | 86.44 | 11.10 | 119.28 | 17.80 |
| AD-1706293.1 | 61.24 | 25.25 | 76.63 | 12.52 | 130.80 | 28.10 |
| AD-1706290.1 | 62.56 | 11.16 | 95.20 | 16.61 | 116.52 | 11.90 |
| AD-1706383.1 | 64.06 | 16.13 | 86.94 | 9.74 | 112.75 | 11.83 |
| AD-1706291.1 | 64.59 | 12.17 | 108.70 | 24.87 | 106.57 | 31.50 |
| AD-1706280.1 | 65.32 | 6.57 | 91.46 | 16.24 | 108.01 | 13.13 |
| AD-1708140.1 | 65.34 | 2.95 | 87.20 | 9.24 | 106.96 | 6.65 |
| AD-1708146.1 | 65.43 | 11.72 | 84.84 | 10.88 | 112.01 | 18.37 |
| AD-1706313.1 | 66.71 | 16.10 | 94.16 | 19.83 | 100.38 | 27.90 |
| AD-1706408.1 | 67.80 | 19.49 | 71.97 | 16.90 | 125.25 | 16.19 |
| AD-1708120.1 | 68.00 | 23.76 | 81.05 | 8.12 | 121.03 | 26.25 |
| AD-1711741.1 | 68.04 | 23.87 | 87.67 | 14.56 | 132.56 | 17.04 |
| AD-1708151.1 | 69.02 | 16.00 | 89.83 | 4.84 | 118.89 | 22.05 |
| AD-1706309.1 | 69.77 | 8.67 | 84.02 | 11.60 | 138.93 | 37.04 |
| AD-1706318.1 | 70.20 | 18.67 | 91.66 | 23.35 | 114.12 | 16.35 |
| AD-1706273.1 | 70.34 | 15.43 | 134.72 | 59.02 | 106.63 | 19.73 |
| AD-1708156.1 | 70.85 | 5.13 | 94.15 | 10.42 | 122.14 | 12.71 |
| AD-1706310.1 | 70.85 | 8.64 | 92.35 | 8.01 | 109.09 | 13.66 |
| AD-1708143.1 | 72.22 | 46.65 | 74.07 | 2.75 | 99.36 | 33.10 |
| AD-1706270.1 | 73.33 | 11.55 | 107.41 | 33.72 | 104.81 | 18.07 |
| AD-1708245.1 | 74.06 | 11.02 | 91.64 | 18.81 | 115.07 | 18.23 |
| AD-1708148.1 | 75.43 | 10.51 | 110.24 | 42.78 | 127.20 | 18.92 |
| AD-1706288.1 | 78.50 | 9.54 | 106.30 | 33.79 | 112.08 | 17.34 |
| AD-1706269.1 | 78.75 | 9.19 | 97.34 | 9.54 | 94.15 | 26.99 |
| AD-1706387.1 | 79.56 | 16.72 | 90.68 | 20.17 | 99.57 | 33.24 |
| AD-1708126.1 | 81.86 | 10.41 | 89.72 | 13.80 | 110.36 | 19.57 |
| AD-1706268.1 | 82.69 | 6.38 | 87.59 | 10.70 | 91.80 | 35.13 |
| AD-1708149.1 | 83.56 | 16.19 | 87.38 | 11.09 | 116.42 | 10.39 |
| AD-1706283.1 | 83.95 | 15.44 | 114.55 | 19.46 | 110.69 | 12.30 |
| AD-1708118.1 | 83.97 | 11.32 | 78.18 | 3.60 | 92.20 | 9.25 |
| AD-1708166.1 | 84.10 | 9.90 | 120.20 | 18.18 | 104.28 | 12.44 |
| AD-1711742.1 | 84.46 | 23.20 | 96.26 | 12.03 | 105.58 | 8.27 |
| AD-1708145.1 | 84.74 | 24.20 | 97.39 | 17.29 | 113.55 | 20.45 |
| AD-1706282.1 | 85.07 | 19.02 | 95.14 | 23.04 | 120.96 | 13.71 |
| AD-1708135.1 | 87.22 | 17.87 | 100.41 | 17.38 | 108.11 | 12.87 |
| AD-1706314.1 | 87.98 | 27.86 | 94.95 | 10.60 | 118.63 | 19.08 |
| AD-1706381.1 | 90.73 | 25.96 | 112.16 | 35.12 | 117.51 | 25.58 |
| AD-1706311.1 | 92.14 | 18.67 | 116.08 | 9.92 | 129.88 | 24.01 |
| AD-1706384.1 | 93.18 | 20.64 | 101.34 | 25.11 | 104.37 | 25.79 |
| AD-1706385.1 | 93.50 | 27.94 | 105.31 | 39.58 | 104.75 | 6.41 |
| AD-1706308.1 | 94.84 | 25.04 | 103.73 | 23.79 | 113.80 | 17.87 |
| AD-1706338.1 | 95.49 | 12.86 | 98.18 | 6.39 | 136.37 | 13.09 |
| AD-1708132.1 | 96.59 | 16.59 | 110.68 | 23.93 | 127.48 | 24.20 |
| AD-1706380.1 | 96.60 | 21.61 | 90.75 | 13.70 | 111.95 | 26.19 |
| AD-1706407.1 | 97.98 | 15.10 | 117.50 | 33.82 | 125.93 | 8.66 |
| AD-1708170.1 | 98.46 | 11.64 | 116.36 | 41.97 | 132.15 | 17.12 |
| AD-1706337.1 | 98.84 | 10.33 | 101.92 | 12.86 | 124.77 | 20.13 |
| AD-1706272.1 | 99.84 | 23.62 | 102.80 | 15.05 | 118.13 | 8.50 |
| AD-1706386.1 | 100.40 | 14.63 | 96.49 | 11.10 | 119.90 | 15.42 |
| AD-1706271.1 | 101.04 | 6.69 | 99.68 | 8.67 | 133.49 | 59.61 |
| AD-1708223.1 | 101.44 | 30.82 | 98.53 | 16.39 | 117.67 | 7.67 |
| AD-1706276.1 | 101.53 | 28.71 | 96.11 | 25.46 | 115.66 | 33.58 |
| AD-1708124.1 | 102.72 | 19.13 | 102.79 | 8.17 | 120.68 | 21.81 |
| AD-1706277.1 | 103.69 | 22.61 | 112.76 | 19.13 | 90.36 | 25.09 |
| AD-1706303.1 | 103.98 | 5.20 | 97.67 | 13.09 | 113.42 | 28.98 |
| AD-1706279.1 | 104.53 | 24.13 | 99.52 | 8.70 | 122.09 | 25.99 |
| AD-1708225.1 | 104.78 | 20.86 | 105.12 | 19.62 | 108.70 | 16.14 |
| AD-1708127.1 | 106.25 | 18.25 | 114.08 | 21.32 | 118.31 | 11.91 |
| AD-1708227.1 | 106.53 | 19.55 | 107.67 | 15.93 | 120.02 | 29.17 |
| AD-1706388.1 | 106.60 | 11.81 | 110.39 | 11.58 | 121.56 | 34.41 |
| AD-1706281.1 | 107.68 | 6.05 | 98.05 | 9.70 | 127.45 | 22.05 |
| AD-1706266.1 | 107.82 | 19.26 | 98.57 | 12.19 | 110.48 | 5.27 |
| AD-1706312.1 | 108.02 | 20.50 | 107.33 | 27.61 | 118.30 | 21.42 |
| AD-1708163.1 | 109.49 | 8.50 | 95.21 | 3.04 | 100.00 | 28.35 |
| AD-1708130.1 | 112.07 | 20.84 | 114.29 | 5.99 | 135.90 | 28.66 |
| AD-1708220.1 | 114.56 | 17.32 | 93.03 | 8.40 | 127.48 | 21.09 |
| AD-1706274.1 | 117.23 | 47.42 | 95.56 | 7.23 | 95.48 | 3.56 |
| AD-1706275.1 | 117.99 | 17.97 | 122.96 | 24.59 | 102.24 | 29.51 |
| AD-1706265.1 | 119.46 | 15.25 | 106.15 | 16.69 | 130.11 | 27.52 |
| AD-1706278.1 | 122.93 | 41.94 | 93.47 | 14.43 | 116.53 | 8.92 |
| AD-1706382.1 | 130.38 | 29.68 | 115.88 | 31.75 | 114.07 | 27.35 |
| AD-1706642.1 | 9.07 | 1.33 | 33.95 | 3.33 | 73.03 | 9.20 |
| AD-1706584.1 | 11.10 | 1.36 | 39.89 | 8.90 | 62.11 | 12.60 |
| AD-1711744.1 | 14.39 | 2.21 | 50.24 | 8.89 | 79.57 | 19.84 |
| AD-1706588.1 | 14.86 | 1.91 | 40.60 | 4.38 | 72.24 | 12.92 |
| AD-1706583.1 | 16.40 | 0.91 | 54.91 | 8.04 | 90.07 | 11.50 |
| AD-1706591.1 | 16.85 | 2.64 | 45.21 | 8.78 | 75.32 | 9.41 |
| AD-1706593.1 | 16.97 | 1.82 | 62.83 | 22.14 | 82.42 | 26.66 |
| AD-1706587.1 | 17.58 | 1.56 | 58.63 | 25.19 | 80.99 | 11.23 |
| AD-1706547.1 | 17.83 | 3.49 | 53.09 | 6.40 | 81.19 | 10.29 |
| AD-1708399.1 | 17.89 | 2.66 | 59.18 | 20.24 | 81.63 | 18.75 |
| AD-1706548.1 | 17.90 | 0.67 | 52.53 | 5.0 | 96.83 | 14.58 |
| AD-1708368.1 | 21.79 | 3.78 | 74.77 | 6.39 | 117.69 | 7.52 |
| AD-1706640.1 | 23.16 | 2.61 | 60.43 | 5.36 | 91.90 | 28.74 |
| AD-1706641.1 | 23.47 | 7.60 | 61.82 | 8.03 | 86.84 | 9.06 |
| AD-1708357.1 | 24.90 | 5.49 | 64.54 | 8.39 | 99.70 | 20.16 |
| AD-1706524.1 | 25.32 | 5.84 | 71.07 | 11.04 | 105.11 | 25.09 |
| AD-1706585.1 | 26.06 | 3.02 | 64.13 | 14.21 | 104.94 | 17.17 |
| AD-1706590.1 | 27.08 | 6.74 | 90.13 | 35.33 | 97.84 | 35.28 |
| AD-1708407.1 | 27.22 | 9.42 | 61.08 | 8.83 | 82.76 | 13.93 |
| AD-1706594.1 | 28.01 | 3.55 | 68.81 | 7.80 | 93.18 | 9.04 |
| AD-1706414.1 | 28.37 | 4.53 | 58.04 | 13.63 | 87.06 | 19.77 |
| AD-1706538.1 | 28.83 | 3.95 | 87.81 | 10.78 | 103.20 | 18.94 |
| AD-1708251.1 | 29.62 | 3.42 | 61.88 | 15.03 | 91.39 | 20.22 |
| AD-1706546.1 | 30.40 | 3.43 | 74.52 | 6.92 | 108.55 | 16.73 |
| AD-1706543.1 | 32.12 | 2.74 | 73.00 | 8.20 | 108.30 | 25.23 |
| AD-1706536.1 | 32.84 | 7.47 | 64.01 | 14.33 | 92.38 | 12.26 |
| AD-1706545.1 | 34.25 | 3.85 | 62.46 | 5.73 | 98.45 | 9.25 |
| AD-1706520.1 | 35.55 | 5.51 | 81.69 | 15.12 | 110.53 | 13.75 |
| AD-1706582.1 | 35.83 | 6.13 | 61.53 | 16.96 | 94.95 | 16.97 |
| AD-1706525.1 | 36.55 | 4.29 | 94.16 | 23.25 | 91.09 | 12.66 |
| AD-1708369.1 | 38.82 | 21.75 | 70.72 | 3.62 | 97.58 | 17.63 |
| AD-1708255.1 | 39.07 | 5.04 | 67.52 | 11.96 | 90.89 | 8.17 |
| AD-1706535.1 | 39.19 | 8.70 | 104.28 | 31.11 | 97.46 | 6.66 |
| AD-1706415.1 | 39.83 | 5.22 | 55.55 | 6.77 | 80.55 | 9.38 |
| AD-1706544.1 | 39.98 | 3.88 | 60.76 | 7.29 | 80.11 | 18.18 |
| AD-1706639.1 | 42.73 | 3.36 | 79.43 | 15.67 | 101.15 | 20.49 |
| AD-1706550.1 | 42.82 | 15.97 | 94.02 | 43.73 | 107.95 | 26.18 |
| AD-1708248.1 | 43.04 | 3.60 | 68.74 | 8.58 | 93.13 | 19.62 |
| AD-1708365.1 | 43.52 | 8.08 | 106.40 | 35.35 | 101.61 | 17.99 |
| AD-1706589.1 | 43.66 | 11.75 | 73.57 | 3.09 | 103.81 | 11.11 |
| AD-1706523.1 | 43.71 | 6.67 | 91.11 | 14.54 | 116.01 | 5.91 |
| AD-1706586.1 | 44.05 | 8.35 | 99.17 | 34.89 | 102.65 | 24.81 |
| AD-1706412.1 | 44.67 | 9.33 | 82.62 | 12.75 | 95.04 | 15.60 |
| AD-1706420.1 | 45.19 | 4.57 | 71.65 | 14.31 | 101.49 | 40.85 |
| AD-1708401.1 | 46.32 | 4.32 | 109.46 | 36.49 | 137.97 | 46.10 |
| AD-1706537.1 | 47.21 | 3.01 | 83.85 | 7.81 | 113.39 | 14.02 |
| AD-1708397.1 | 47.56 | 4.70 | 82.21 | 16.77 | 103.46 | 14.37 |
| AD-1706592.1 | 49.61 | 12.76 | 74.80 | 17.42 | 103.97 | 30.99 |
| AD-1706539.1 | 50.47 | 7.14 | 64.47 | 6.46 | 81.84 | 2.50 |
| AD-1706532.1 | 51.25 | 5.15 | 79.01 | 18.04 | 96.02 | 7.54 |
| AD-1706580.1 | 51.56 | 13.09 | 58.56 | 10.84 | 100.76 | 23.90 |
| AD-1706413.1 | 51.63 | 5.56 | 67.89 | 6.33 | 81.80 | 12.55 |
| AD-1706419.1 | 54.23 | 19.04 | 93.19 | 30.98 | 121.41 | 34.52 |
| AD-1706581.1 | 54.84 | 7.00 | 71.33 | 14.93 | 84.09 | 14.17 |
| AD-1708447.1 | 54.87 | 13.52 | 104.94 | 21.63 | 118.08 | 18.05 |
| AD-1706579.1 | 54.95 | 7.90 | 70.62 | 4.25 | 104.36 | 27.78 |
| AD-1708408.1 | 56.12 | 12.89 | 90.61 | 8.36 | 99.13 | 20.61 |
| AD-1706549.1 | 56.65 | 9.42 | 81.71 | 9.47 | 97.16 | 9.63 |
| AD-1708342.1 | 56.93 | 7.61 | 80.26 | 12.73 | 106.80 | 25.07 |
| AD-1706636.1 | 57.82 | 5.41 | 85.94 | 12.63 | 99.00 | 13.47 |
| AD-1706425.1 | 58.29 | 2.96 | 76.30 | 14.61 | 95.32 | 14.22 |
| AD-1706578.1 | 60.60 | 13.60 | 87.09 | 12.39 | 109.98 | 28.08 |
| AD-1706418.1 | 63.28 | 10.61 | 96.91 | 13.82 | 111.41 | 24.53 |
| AD-1708356.1 | 64.77 | 12.61 | 78.57 | 18.09 | 99.15 | 15.61 |
| AD-1708347.1 | 65.00 | 17.57 | 82.04 | 19.82 | 95.21 | 15.90 |
| AD-1706424.1 | 65.38 | 7.09 | 85.09 | 16.40 | 105.96 | 30.35 |
| AD-1706417.1 | 66.98 | 12.66 | 85.33 | 20.21 | 111.43 | 27.23 |
| AD-1706521.1 | 68.72 | 12.27 | 90.93 | 12.68 | 119.35 | 25.53 |
| AD-1706530.1 | 70.98 | 12.28 | 86.64 | 9.70 | 94.88 | 14.24 |
| AD-1706522.1 | 71.46 | 9.68 | 106.53 | 20.33 | 92.61 | 7.37 |
| AD-1706527.1 | 71.63 | 5.56 | 97.88 | 24.77 | 112.26 | 31.32 |
| AD-1706416.1 | 72.34 | 8.83 | 80.67 | 4.08 | 98.15 | 20.87 |
| AD-1706534.1 | 76.28 | 8.47 | 81.77 | 15.66 | 99.83 | 24.74 |
| AD-1706421.1 | 79.32 | 6.14 | 93.79 | 19.12 | 122.72 | 30.72 |
| AD-1711743.1 | 80.13 | 9.62 | 88.65 | 8.30 | 93.61 | 12.47 |
| AD-1706635.1 | 80.53 | 12.44 | 85.68 | 19.07 | 105.70 | 21.04 |
| AD-1706528.1 | 81.09 | 5.66 | 73.66 | 5.18 | 122.46 | 19.28 |
| AD-1706637.1 | 82.19 | 27.49 | 90.88 | 12.79 | 111.05 | 20.25 |
| AD-1708403.1 | 83.00 | 19.04 | 92.02 | 7.56 | 116.05 | 23.70 |
| AD-1708405.1 | 85.44 | 23.49 | 103.54 | 43.41 | 116.29 | 44.00 |
| AD-1706533.1 | 85.51 | 14.92 | 115.23 | 27.84 | 101.95 | 17.97 |
| AD-1708344.1 | 85.73 | 11.66 | 97.48 | 5.76 | 110.94 | 21.22 |
| AD-1708361.1 | 86.28 | 9.76 | 88.85 | 15.65 | 109.24 | 31.96 |
| AD-1706423.1 | 88.17 | 11.15 | 84.06 | 8.73 | 122.58 | 38.31 |
| AD-1708260.1 | 88.99 | 5.12 | 94.37 | 17.05 | 114.30 | 29.22 |
| AD-1708350.1 | 90.61 | 12.13 | 85.27 | 9.24 | 100.73 | 9.67 |
| AD-1706638.1 | 95.90 | 12.95 | 85.50 | 18.85 | 110.68 | 17.30 |
| AD-1708402.1 | 99.66 | 13.44 | 91.37 | 13.19 | 111.56 | 29.33 |
| AD-1706526.1 | 101.27 | 3.70 | 93.41 | 11.90 | 113.66 | 19.87 |
| AD-1708249.1 | 102.18 | 16.36 | 97.35 | 19.73 | 116.55 | 34.10 |
| AD-1706661.1 | 6.60 | 0.84 | 74.72 | 4.36 | 110.66 | 17.49 |
| AD-1708473.1 | 6.62 | 1.40 | 69.52 | 3.82 | 108.89 | 15.87 |
| AD-1706665.1 | 6.77 | 0.79 | 67.01 | 13.10 | 103.64 | 16.11 |
| AD-1706650.1 | 7.00 | 1.13 | 70.73 | 11.82 | 108.62 | 8.15 |
| AD-1706645.1 | 7.25 | 3.64 | 74.87 | 8.06 | 107.77 | 11.83 |
| AD-1706653.1 | 7.74 | 1.35 | 76.42 | 7.69 | 113.74 | 17.40 |
| AD-1711749.1 | 8.54 | 1.25 | 68.74 | 2.15 | 106.66 | 11.71 |
| AD-1706763.1 | 8.70 | 2.21 | 72.90 | 15.62 | 107.52 | 10.92 |
| AD-1708478.1 | 9.57 | 1.33 | 78.24 | 7.40 | 107.29 | 10.61 |
| AD-1706749.1 | 10.07 | 1.20 | 74.95 | 9.09 | 99.46 | 4.18 |
| AD-1706643.1 | 10.53 | 1.64 | 73.09 | 9.66 | 90.78 | 10.00 |
| AD-1706666.1 | 13.18 | 2.44 | 80.01 | 9.39 | 106.83 | 5.84 |
| AD-1706796.1 | 15.63 | 3.43 | 89.88 | 10.35 | 103.93 | 11.87 |
| AD-1706667.1 | 17.36 | 4.27 | 101.51 | 16.36 | 111.65 | 19.15 |
| AD-1706671.1 | 17.42 | 2.37 | 90.42 | 16.21 | 103.59 | 6.63 |
| AD-1706662.1 | 18.09 | 2.45 | 95.05 | 11.73 | 105.69 | 13.23 |
| AD-1706660.1 | 18.70 | 4.24 | 91.56 | 4.98 | 105.45 | 10.46 |
| AD-1706668.1 | 19.20 | 2.47 | 80.70 | 4.94 | 104.90 | 16.86 |
| AD-1706678.1 | 19.89 | 4.19 | 95.24 | 9.95 | 106.34 | 7.14 |
| AD-1706761.1 | 20.09 | 5.94 | 103.36 | 8.72 | 110.93 | 4.49 |
| AD-1706729.1 | 20.69 | 1.20 | 82.71 | 9.83 | 98.57 | 7.22 |
| AD-1706758.1 | 20.84 | 2.84 | 97.40 | 12.07 | 111.12 | 8.71 |
| AD-1706802.1 | 21.81 | 2.61 | 87.99 | 12.58 | 104.85 | 6.50 |
| AD-1706762.1 | 21.86 | 1.22 | 87.78 | 3.24 | 106.95 | 10.71 |
| AD-1706673.1 | 22.52 | 4.61 | 95.13 | 17.07 | 106.48 | 11.75 |
| AD-1706656.1 | 24.59 | 2.05 | 83.54 | 7.22 | 100.64 | 2.81 |
| AD-1706654.1 | 24.83 | 1.15 | 109.60 | 22.66 | 105.15 | 8.80 |
| AD-1706651.1 | 25.30 | 2.94 | 93.94 | 14.27 | 112.44 | 11.34 |
| AD-1708561.1 | 25.60 | 3.99 | 104.77 | 12.81 | 102.89 | 6.87 |
| AD-1706644.1 | 26.90 | 0.85 | 84.09 | 3.99 | 101.42 | 5.45 |
| AD-1706674.1 | 28.45 | 3.09 | 88.45 | 9.49 | 104.73 | 2.90 |
| AD-1706759.1 | 29.16 | 20.00 | 85.18 | 8.32 | 110.91 | 17.22 |
| AD-1711747.1 | 29.23 | 3.34 | 92.97 | 13.24 | 109.77 | 11.44 |
| AD-1706655.1 | 29.64 | 16.36 | 94.04 | 14.12 | 106.67 | 15.84 |
| AD-1706755.1 | 29.74 | 4.96 | 90.27 | 13.45 | 89.74 | 8.63 |
| AD-1706750.1 | 29.94 | 9.26 | 87.13 | 8.39 | 109.44 | 12.06 |
| AD-1706756.1 | 29.99 | 0.59 | 92.61 | 17.60 | 106.44 | 15.86 |
| AD-1706799.1 | 30.02 | 5.93 | 97.59 | 14.48 | 111.47 | 11.09 |
| AD-1708485.1 | 30.12 | 11.59 | 89.97 | 7.46 | 105.61 | 12.03 |
| AD-1706652.1 | 31.17 | 4.31 | 94.61 | 4.74 | 105.02 | 6.14 |
| AD-1706649.1 | 32.31 | 8.03 | 95.79 | 12.11 | 97.70 | 9.45 |
| AD-1706764.1 | 32.90 | 0.57 | 96.72 | 28.52 | 109.38 | 11.25 |
| AD-1706760.1 | 33.00 | 4.03 | 89.53 | 11.65 | 101.22 | 11.01 |
| AD-1706748.1 | 35.18 | 4.35 | 96.77 | 10.82 | 110.03 | 9.91 |
| AD-1711750.1 | 37.69 | 7.93 | 98.35 | 9.29 | 106.27 | 9.06 |
| AD-1708457.1 | 38.56 | 5.40 | 93.19 | 5.67 | 107.79 | 12.22 |
| AD-1706751.1 | 39.44 | 19.99 | 94.73 | 9.40 | 109.19 | 8.46 |
| AD-1708454.1 | 40.71 | 3.27 | 89.75 | 12.18 | 107.44 | 15.74 |
| AD-1706753.1 | 40.99 | 9.16 | 100.89 | 28.03 | 110.69 | 15.45 |
| AD-1708564.1 | 41.34 | 4.73 | 88.93 | 5.25 | 95.35 | 1.26 |
| AD-1706746.1 | 42.57 | 6.65 | 88.82 | 8.77 | 107.73 | 13.47 |
| AD-1708464.1 | 42.62 | 3.80 | 95.60 | 4.87 | 104.61 | 11.22 |
| AD-1706672.1 | 43.78 | 10.25 | 95.27 | 10.60 | 98.19 | 9.52 |
| AD-1711746.1 | 45.65 | 2.07 | 112.78 | 25.92 | 102.47 | 13.57 |
| AD-1708483.1 | 48.04 | 7.38 | 101.48 | 24.43 | 102.75 | 7.74 |
| AD-1708565.1 | 48.61 | 10.48 | 99.08 | 14.13 | 99.70 | 5.83 |
| AD-1706797.1 | 48.74 | 5.96 | 90.55 | 14.02 | 100.91 | 9.04 |
| AD-1711748.1 | 50.55 | 10.58 | 104.32 | 12.32 | 105.92 | 11.82 |
| AD-1706646.1 | 50.78 | 11.75 | 105.91 | 14.08 | 101.10 | 6.21 |
| AD-1708468.1 | 50.98 | 6.40 | 99.67 | 13.25 | 111.96 | 9.91 |
| AD-1706664.1 | 51.96 | 13.92 | 90.04 | 8.66 | 108.53 | 15.23 |
| AD-1706747.1 | 52.03 | 8.41 | 103.06 | 21.38 | 114.38 | 11.30 |
| AD-1706765.1 | 53.56 | 7.63 | 93.88 | 18.00 | 95.27 | 4.65 |
| AD-1706800.1 | 53.95 | 8.49 | 95.50 | 8.96 | 107.45 | 9.95 |
| AD-1708462.1 | 54.04 | 6.29 | 92.64 | 7.40 | 118.65 | 14.56 |
| AD-1706752.1 | 55.51 | 8.14 | 88.82 | 6.13 | 97.91 | 7.54 |
| AD-1706745.1 | 57.43 | 9.49 | 93.02 | 5.38 | 97.74 | 7.60 |
| AD-1706669.1 | 63.72 | 7.32 | 90.06 | 8.80 | 102.31 | 8.84 |
| AD-1708466.1 | 64.64 | 14.05 | 95.96 | 16.75 | 100.99 | 12.25 |
| AD-1706675.1 | 64.77 | 2.67 | 100.96 | 6.87 | 108.17 | 9.63 |
| AD-1711751.1 | 65.52 | 14.01 | 99.40 | 20.89 | 95.72 | 19.18 |
| AD-1711745.1 | 68.13 | 7.56 | 101.21 | 11.15 | 100.69 | 4.58 |
| AD-1706803.1 | 73.64 | 8.01 | 92.32 | 11.60 | 104.35 | 10.44 |
| AD-1706754.1 | 73.79 | 5.20 | 88.05 | 10.42 | 101.75 | 12.74 |
| AD-1708482.1 | 75.95 | 14.79 | 99.22 | 6.65 | 99.58 | 19.78 |
| AD-1706801.1 | 78.50 | 13.33 | 93.60 | 9.95 | 100.32 | 4.13 |
| AD-1708458.1 | 78.70 | 7.33 | 87.84 | 4.93 | 103.98 | 9.93 |
| AD-1711752.1 | 79.38 | 12.59 | 82.60 | 8.23 | 97.25 | 4.37 |
| AD-1708567.1 | 80.84 | 18.53 | 93.23 | 9.30 | 100.82 | 7.56 |
| AD-1706804.1 | 81.90 | 4.96 | 97.61 | 18.31 | 104.98 | 5.27 |
| AD-1706670.1 | 83.22 | 11.36 | 101.97 | 12.99 | 99.92 | 6.96 |
| AD-1706732.1 | 85.31 | 21.66 | 96.44 | 10.50 | 104.05 | 12.80 |
| AD-1708647.1 | 85.35 | 15.28 | 119.04 | 41.38 | 108.04 | 20.54 |
| AD-1706798.1 | 85.62 | 7.06 | 103.36 | 13.05 | 100.71 | 15.44 |
| AD-1708476.1 | 88.09 | 5.36 | 99.39 | 9.61 | 107.56 | 11.68 |
| AD-1708650.1 | 89.03 | 12.02 | 113.51 | 14.13 | 106.25 | 8.75 |
| AD-1706735.1 | 92.62 | 7.77 | 111.07 | 11.08 | 108.42 | 10.58 |
| AD-1706731.1 | 94.79 | 6.67 | 97.21 | 9.56 | 99.62 | 3.81 |
| AD-1708559.1 | 95.00 | 7.62 | 100.14 | 12.07 | 102.77 | 7.98 |
| AD-1708648.1 | 108.92 | 28.51 | 99.75 | 9.85 | 106.66 | 6.01 |
| AD-1706854.1 | 25.37 | 3.01 | 103.72 | 47.41 | 103.23 | 9.61 |
| AD-1708766.1 | 26.83 | 6.17 | 96.07 | 18.36 | 102.24 | 7.86 |
| AD-1706929.1 | 27.18 | 8.06 | 75.76 | 7.80 | 97.46 | 18.20 |
| AD-1706934.1 | 27.89 | 6.08 | 92.28 | 12.84 | 98.05 | 12.81 |
| AD-1706879.1 | 35.05 | 3.81 | 88.34 | 13.15 | 104.53 | 21.27 |
| AD-1706833.1 | 35.80 | 6.70 | 95.10 | 10.15 | 105.49 | 8.37 |
| AD-1706856.1 | 36.63 | 4.23 | 110.20 | 26.30 | 107.65 | 12.80 |
| AD-1706858.1 | 37.72 | 6.21 | 81.87 | 12.77 | 92.77 | 14.43 |
| AD-1706930.1 | 40.50 | 1.61 | 108.05 | 35.35 | 86.82 | 30.20 |
| AD-1706875.1 | 40.60 | 4.27 | 118.37 | 25.42 | 98.68 | 15.75 |
| AD-1706855.1 | 41.18 | 11.54 | 131.44 | 55.87 | 98.30 | 19.73 |
| AD-1706863.1 | 43.26 | 4.15 | 100.28 | 15.63 | 92.69 | 21.34 |
| AD-1706933.1 | 43.61 | 9.36 | 102.54 | 30.69 | 104.09 | 5.81 |
| AD-1706849.1 | 47.30 | 6.81 | 102.81 | 19.95 | 84.13 | 29.87 |
| AD-1708707.1 | 50.03 | 11.97 | 108.24 | 34.23 | 104.48 | 11.54 |
| AD-1706831.1 | 52.48 | 7.54 | 115.05 | 20.86 | 102.75 | 17.63 |
| AD-1706861.1 | 52.54 | 16.14 | 101.45 | 26.45 | 108.92 | 30.66 |
| AD-1706878.1 | 53.45 | 9.24 | 104.31 | 24.53 | 108.89 | 11.75 |
| AD-1706860.1 | 54.13 | 8.56 | 93.86 | 12.56 | 107.75 | 22.52 |
| AD-1706876.1 | 54.16 | 11.40 | 102.97 | 37.17 | 100.98 | 9.73 |
| AD-1706922.1 | 54.52 | 11.46 | 116.79 | 22.40 | 97.89 | 10.04 |
| AD-1706877.1 | 54.71 | 8.75 | 124.94 | 31.32 | 111.27 | 15.98 |
| AD-1706910.1 | 56.13 | 5.44 | 100.89 | 10.10 | 99.37 | 24.39 |
| AD-1708724.1 | 56.38 | 11.75 | 113.47 | 33.12 | 96.26 | 14.65 |
| AD-1706851.1 | 56.88 | 8.13 | 99.19 | 16.09 | 100.04 | 9.14 |
| AD-1706848.1 | 57.59 | 19.63 | 88.30 | 19.11 | 108.41 | 23.17 |
| AD-1706862.1 | 57.90 | 17.76 | 100.76 | 21.18 | 98.92 | 19.89 |
| AD-1706873.1 | 60.54 | 4.01 | 77.98 | 11.86 | 83.33 | 21.11 |
| AD-1706925.1 | 61.27 | 5.41 | 141.22 | 45.16 | 112.13 | 14.93 |
| AD-1706807.1 | 63.36 | 6.27 | 119.59 | 35.44 | 106.38 | 16.29 |
| AD-1706808.1 | 63.79 | 6.32 | 81.18 | 2.15 | 83.79 | 14.66 |
| AD-1706913.1 | 64.51 | 5.44 | 86.83 | 10.13 | 102.68 | 11.81 |
| AD-1708771.1 | 65.72 | 13.62 | 73.79 | 9.06 | 99.46 | 12.36 |
| AD-1708657.1 | 66.55 | 5.60 | 90.80 | 25.74 | 100.19 | 11.20 |
| AD-1706852.1 | 66.74 | 13.45 | 110.13 | 42.60 | 109.17 | 10.74 |
| AD-1706908.1 | 68.07 | 13.31 | 114.93 | 23.24 | 97.36 | 4.38 |
| AD-1706927.1 | 69.35 | 9.74 | 96.16 | 18.41 | 110.08 | 10.62 |
| AD-1706932.1 | 69.69 | 5.02 | 104.45 | 21.71 | 97.67 | 11.10 |
| AD-1706834.1 | 70.38 | 6.62 | 84.04 | 8.43 | 99.35 | 17.43 |
| AD-1706918.1 | 71.54 | 17.49 | 113.13 | 10.29 | 114.70 | 17.65 |
| AD-1706866.1 | 71.83 | 14.46 | 88.92 | 33.22 | 106.35 | 14.58 |
| AD-1706983.1 | 73.76 | 8.01 | 112.98 | 24.54 | 86.85 | 26.78 |
| AD-1706859.1 | 74.92 | 24.74 | 90.87 | 21.71 | 100.06 | 13.80 |
| AD-1706982.1 | 76.10 | 19.78 | 106.63 | 36.38 | 98.22 | 13.19 |
| AD-1706835.1 | 77.42 | 15.50 | 104.09 | 23.87 | 101.43 | 22.46 |
| AD-1706921.1 | 77.52 | 19.03 | 100.22 | 19.85 | 98.95 | 16.37 |
| AD-1706989.1 | 78.00 | 22.52 | 93.63 | 12.00 | 92.94 | 19.53 |
| AD-1708746.1 | 79.02 | 19.60 | 114.66 | 35.50 | 100.74 | 15.01 |
| AD-1706850.1 | 79.32 | 13.85 | 91.59 | 5.45 | 94.78 | 15.55 |
| AD-1706985.1 | 80.89 | 13.10 | 88.36 | 16.54 | 108.60 | 10.83 |
| AD-1706923.1 | 81.37 | 21.19 | 106.79 | 11.31 | 103.06 | 13.83 |
| AD-1706909.1 | 81.73 | 14.01 | 116.11 | 47.50 | 105.49 | 14.70 |
| AD-1706811.1 | 81.88 | 17.25 | 92.52 | 16.10 | 101.66 | 13.54 |
| AD-1708813.1 | 85.76 | 27.33 | 115.49 | 21.38 | 109.30 | 9.84 |
| AD-1708748.1 | 87.90 | 14.15 | 86.02 | 8.62 | 100.81 | 2.57 |
| AD-1706984.1 | 88.94 | 16.25 | 113.85 | 25.66 | 108.56 | 14.03 |
| AD-1708697.1 | 89.52 | 10.09 | 120.53 | 38.28 | 113.90 | 9.54 |
| AD-1706911.1 | 89.91 | 17.73 | 101.81 | 15.95 | 95.76 | 15.10 |
| AD-1708709.1 | 90.74 | 14.16 | 100.10 | 17.53 | 107.72 | 14.67 |
| AD-1706853.1 | 91.75 | 13.32 | 113.59 | 26.59 | 113.95 | 29.85 |
| AD-1706926.1 | 96.63 | 6.03 | 95.17 | 15.95 | 103.87 | 30.98 |
| AD-1706832.1 | 97.54 | 16.47 | 104.10 | 33.68 | 107.00 | 11.63 |
| AD-1706987.1 | 98.29 | 12.34 | 95.69 | 20.12 | 98.30 | 10.47 |
| AD-1706809.1 | 98.67 | 26.38 | 99.13 | 15.24 | 82.43 | 14.61 |
| AD-1708700.1 | 99.27 | 21.13 | 106.72 | 9.99 | 96.03 | 19.58 |
| AD-1706988.1 | 101.65 | 24.29 | 106.78 | 29.53 | 106.48 | 20.25 |
| AD-1706914.1 | 101.79 | 11.21 | 90.48 | 4.14 | 100.55 | 13.62 |
| AD-1706931.1 | 103.40 | 66.16 | 107.78 | 26.76 | 101.02 | 24.37 |
| AD-1708683.1 | 104.06 | 8.55 | 125.73 | 42.10 | 107.19 | 5.34 |
| AD-1711754.1 | 104.34 | 15.76 | 114.12 | 40.53 | 105.06 | 9.88 |
| AD-1706836.1 | 106.36 | 12.55 | 100.96 | 22.23 | 103.19 | 32.20 |
| AD-1708662.1 | 107.32 | 8.18 | 88.67 | 10.59 | 96.58 | 9.88 |
| AD-1708759.1 | 107.49 | 28.12 | 89.51 | 14.33 | 101.64 | 19.99 |
| AD-1708680.1 | 108.20 | 15.23 | 97.77 | 24.39 | 102.61 | 12.78 |
| AD-1706874.1 | 109.21 | 20.84 | 100.69 | 38.85 | 100.93 | 14.52 |
| AD-1706864.1 | 110.63 | 16.57 | 101.39 | 23.00 | 101.73 | 22.95 |
| AD-1711753.1 | 111.19 | 14.58 | 93.59 | 17.39 | 110.58 | 13.90 |
| AD-1706912.1 | 111.75 | 15.92 | 105.01 | 18.67 | 108.43 | 10.78 |
| AD-1708751.1 | 112.97 | 12.72 | 116.26 | 23.93 | 104.25 | 11.26 |
| AD-1708721.1 | 114.27 | 20.90 | 100.77 | 14.87 | 104.56 | 12.44 |
| AD-1708819.1 | 116.07 | 29.98 | 91.50 | 9.56 | 112.22 | 10.56 |
| AD-1708698.1 | 117.55 | 77.59 | 94.05 | 14.37 | 72.00 | 40.57 |
| AD-1706857.1 | 118.83 | 19.85 | 117.65 | 40.36 | 108.84 | 4.65 |
| AD-1708704.1 | 119.12 | 34.40 | 128.50 | 32.16 | 109.76 | 22.57 |
| AD-1706812.1 | 119.69 | 43.53 | 122.34 | 33.51 | 87.62 | 24.43 |
| AD-1708719.1 | 122.89 | 21.25 | 107.22 | 23.83 | 99.76 | 9.98 |
| AD-1708706.1 | 127.72 | 37.05 | 95.70 | 14.61 | 99.73 | 16.92 |
| AD-1708764.1 | 131.45 | 28.75 | 107.92 | 18.70 | 105.12 | 19.46 |
| AD-1708763.1 | 137.95 | 34.65 | 119.14 | 40.49 | 113.97 | 7.55 |
| AD-1708711.1 | 175.32 | 14.43 | 126.75 | 39.55 | 116.98 | 7.84 |
| AD-1707061.1 | 8.33 | 1.86 | 88.81 | 10.65 | 90.67 | 24.02 |
| AD-1707058.1 | 9.98 | 1.11 | 94.75 | 9.86 | 101.20 | 14.48 |
| AD-1707063.1 | 12.85 | 3.24 | 99.95 | 14.44 | 104.17 | 16.47 |
| AD-1707050.1 | 13.92 | 2.38 | 93.91 | 11.33 | 98.57 | 8.81 |
| AD-1707060.1 | 13.97 | 2.53 | 92.54 | 10.48 | 120.97 | 7.47 |
| AD-1707075.1 | 16.10 | 2.42 | 99.13 | 4.47 | 111.23 | 16.36 |
| AD-1707017.1 | 17.50 | 2.11 | 99.33 | 5.23 | 109.99 | 17.00 |
| AD-1708882.1 | 18.85 | 3.63 | 101.54 | 16.25 | 108.57 | 19.96 |
| AD-1707086.1 | 19.02 | 2.90 | 101.81 | 13.51 | 110.60 | 8.16 |
| AD-1707088.1 | 19.02 | 1.40 | 105.64 | 12.93 | 111.73 | 30.08 |
| AD-1707087.1 | 19.19 | 2.03 | 105.11 | 7.90 | 115.78 | 14.42 |
| AD-1707053.1 | 19.50 | 3.76 | 94.83 | 13.66 | 109.87 | 21.89 |
| AD-1711760.1 | 20.00 | 1.81 | 88.73 | 4.33 | 110.48 | 5.26 |
| AD-1711761.1 | 21.94 | 3.63 | 97.37 | 7.10 | 120.53 | 32.80 |
| AD-1707051.1 | 22.12 | 1.59 | 105.96 | 10.05 | 112.06 | 12.44 |
| AD-1707084.1 | 22.54 | 7.73 | 105.12 | 14.83 | 112.28 | 15.31 |
| AD-1707085.1 | 22.65 | 1.28 | 97.83 | 9.25 | 123.86 | 10.32 |
| AD-1707054.1 | 23.29 | 2.06 | 99.81 | 8.18 | 109.43 | 12.98 |
| AD-1707052.1 | 23.59 | 2.74 | 94.34 | 13.80 | 111.93 | 13.23 |
| AD-1707095.1 | 25.27 | 3.63 | 104.94 | 27.94 | 110.90 | 14.62 |
| AD-1707092.1 | 25.31 | 6.58 | 92.76 | 19.70 | 101.56 | 14.57 |
| AD-1711756.1 | 25.49 | 5.39 | 96.10 | 8.16 | 104.56 | 9.24 |
| AD-1707062.1 | 25.58 | 5.66 | 92.16 | 4.07 | 117.28 | 15.75 |
| AD-1707047.1 | 25.77 | 8.14 | 99.31 | 12.88 | 114.18 | 9.49 |
| AD-1707070.1 | 27.17 | 2.34 | 94.16 | 12.13 | 119.42 | 12.78 |
| AD-1708899.1 | 27.37 | 5.49 | 100.65 | 3.32 | 111.69 | 14.24 |
| AD-1708902.1 | 27.66 | 3.25 | 92.63 | 7.26 | 106.35 | 19.13 |
| AD-1707098.1 | 28.90 | 9.71 | 92.87 | 12.33 | 96.92 | 27.94 |
| AD-1707066.1 | 32.02 | 8.19 | 94.37 | 9.02 | 107.58 | 12.73 |
| AD-1707083.1 | 34.19 | 13.45 | 97.84 | 13.54 | 126.24 | 32.35 |
| AD-1707057.1 | 34.23 | 5.87 | 100.62 | 7.09 | 121.36 | 13.17 |
| AD-1707059.1 | 34.79 | 4.28 | 108.93 | 6.94 | 125.08 | 3.39 |
| AD-1707018.1 | 36.61 | 3.49 | 112.84 | 18.20 | 103.78 | 12.48 |
| AD-1707067.1 | 37.21 | 9.85 | 101.33 | 2.41 | 109.01 | 6.56 |
| AD-1711758.1 | 37.55 | 1.79 | 95.70 | 13.53 | 101.50 | 11.23 |
| AD-1707082.1 | 39.65 | 9.15 | 98.31 | 8.81 | 107.83 | 11.35 |
| AD-1707076.1 | 40.68 | 4.39 | 100.27 | 1.83 | 120.22 | 15.13 |
| AD-1707048.1 | 41.96 | 8.19 | 104.43 | 20.77 | 100.43 | 17.11 |
| AD-1707019.1 | 42.99 | 5.23 | 99.22 | 6.55 | 107.64 | 17.16 |
| AD-1708890.1 | 43.91 | 18.14 | 100.48 | 14.09 | 98.26 | 19.93 |
| AD-1707093.1 | 44.02 | 1.40 | 105.14 | 8.37 | 117.58 | 5.28 |
| AD-1708911.1 | 44.15 | 7.91 | 95.63 | 15.64 | 108.40 | 21.86 |
| AD-1707105.1 | 44.17 | 8.08 | 110.35 | 11.53 | 115.89 | 15.69 |
| AD-1707064.1 | 44.50 | 3.66 | 102.70 | 9.58 | 110.63 | 13.42 |
| AD-1707074.1 | 44.84 | 9.51 | 101.54 | 7.85 | 104.90 | 13.69 |
| AD-1707056.1 | 46.78 | 10.10 | 102.19 | 11.68 | 113.06 | 22.60 |
| AD-1711762.1 | 49.02 | 6.03 | 97.20 | 2.29 | 107.59 | 3.47 |
| AD-1707078.1 | 52.73 | 5.48 | 107.20 | 15.35 | 115.13 | 11.87 |
| AD-1707030.1 | 53.66 | 6.53 | 111.11 | 14.02 | 113.90 | 15.90 |
| AD-1707077.1 | 53.70 | 8.63 | 101.57 | 13.11 | 111.56 | 8.32 |
| AD-1707065.1 | 53.74 | 1.99 | 108.29 | 8.35 | 122.33 | 12.73 |
| AD-1707127.1 | 54.77 | 8.44 | 105.15 | 8.40 | 108.70 | 12.81 |
| AD-1707094.1 | 57.47 | 8.53 | 108.23 | 12.77 | 105.37 | 10.86 |
| AD-1707020.1 | 58.01 | 13.48 | 101.09 | 15.13 | 110.62 | 22.22 |
| AD-1707021.1 | 58.79 | 18.20 | 124.68 | 43.20 | 100.48 | 22.35 |
| AD-1707080.1 | 58.94 | 7.31 | 104.10 | 9.25 | 116.61 | 6.61 |
| AD-1707126.1 | 59.59 | 9.32 | 96.42 | 13.31 | 109.61 | 12.60 |
| AD-1708908.1 | 59.96 | 11.47 | 105.79 | 7.21 | 205.27 | 163.17 |
| AD-1707125.1 | 60.35 | 4.53 | 103.79 | 11.65 | 116.67 | 11.23 |
| AD-1708901.1 | 60.42 | 16.23 | 92.03 | 15.94 | 119.73 | 19.06 |
| AD-1711755.1 | 60.73 | 5.54 | 97.60 | 4.64 | 113.80 | 6.04 |
| AD-1707055.1 | 61.12 | 9.30 | 96.50 | 10.89 | 102.20 | 22.76 |
| AD-1707014.1 | 61.49 | 4.18 | 99.29 | 8.04 | 109.12 | 13.68 |
| AD-1707023.1 | 62.68 | 15.83 | 97.52 | 15.74 | 104.31 | 8.10 |
| AD-1706991.1 | 64.23 | 11.46 | 88.71 | 9.37 | 95.92 | 12.46 |
| AD-1707081.1 | 64.73 | 13.16 | 107.14 | 11.08 | 112.73 | 29.65 |
| AD-1708872.1 | 66.53 | 19.46 | 88.90 | 9.68 | 112.87 | 12.88 |
| AD-1707097.1 | 67.80 | 12.95 | 105.41 | 12.10 | 108.88 | 12.85 |
| AD-1708907.1 | 69.34 | 5.34 | 111.02 | 6.94 | 103.77 | 12.88 |
| AD-1707013.1 | 70.83 | 20.94 | 107.47 | 7.74 | 94.44 | 11.05 |
| AD-1707015.1 | 71.33 | 23.32 | 105.50 | 8.59 | 106.57 | 16.42 |
| AD-1708887.1 | 71.55 | 15.47 | 106.33 | 10.29 | 110.30 | 17.14 |
| AD-1707129.1 | 71.65 | 20.96 | 103.58 | 6.43 | 91.33 | 26.27 |
| AD-1708844.1 | 72.88 | 12.10 | 116.14 | 37.29 | 110.62 | 3.95 |
| AD-1708856.1 | 73.95 | 18.67 | 103.53 | 5.03 | 80.24 | 39.02 |
| AD-1708875.1 | 76.60 | 11.35 | 99.59 | 15.59 | 135.66 | 18.88 |
| AD-1708889.1 | 79.17 | 11.66 | 110.70 | 17.57 | 120.98 | 7.68 |
| AD-1707049.1 | 80.31 | 5.85 | 108.76 | 7.19 | 119.03 | 5.82 |
| AD-1711763.1 | 84.78 | 21.20 | 99.75 | 14.69 | 113.14 | 14.33 |
| AD-1707024.1 | 87.28 | 6.38 | 93.68 | 12.29 | 108.98 | 14.26 |
| AD-1707022.1 | 87.29 | 14.92 | 98.16 | 14.55 | 102.07 | 11.52 |
| AD-1707128.1 | 87.44 | 2.93 | 94.65 | 5.66 | 109.94 | 20.88 |
| AD-1711759.1 | 88.12 | 18.24 | 106.57 | 14.25 | 111.09 | 6.10 |
| AD-1707016.1 | 88.23 | 17.94 | 94.55 | 2.56 | 104.53 | 16.40 |
| AD-1711757.1 | 88.63 | 12.42 | 95.80 | 12.28 | 122.43 | 7.14 |
| AD-1708842.1 | 89.93 | 21.36 | 125.99 | 41.26 | 125.37 | 9.77 |
| AD-1708849.1 | 91.50 | 19.06 | 116.08 | 31.45 | 79.83 | 13.17 |
| AD-1706990.1 | 91.94 | 11.09 | 125.77 | 47.09 | 109.78 | 6.85 |
| AD-1708921.1 | 112.06 | 22.19 | 112.08 | 21.18 | 113.33 | 20.65 |
| AD-1707025.1 | 122.67 | 6.40 | 108.76 | 12.64 | 120.07 | 12.98 |
| AD-1707152.1 | 13.98 | 2.96 | 115.15 | 23.84 | 101.84 | 9.78 |
| AD-1708956.1 | 16.83 | 1.85 | 87.80 | 11.44 | 100.21 | 20.00 |
| AD-1707143.1 | 17.33 | 4.80 | 104.40 | 35.77 | 110.39 | 5.62 |
| AD-1707136.1 | 17.36 | 2.50 | 98.63 | 18.72 | 82.73 | 17.99 |
| AD-1707146.1 | 20.98 | 3.84 | 108.41 | 18.98 | 107.36 | 8.80 |
| AD-1707132.1 | 22.58 | 2.33 | 97.25 | 9.13 | 105.94 | 17.38 |
| AD-1707161.1 | 22.68 | 5.03 | 89.57 | 15.63 | 96.67 | 20.00 |
| AD-1707170.1 | 23.62 | 2.34 | 95.73 | 12.86 | 95.31 | 5.70 |
| AD-1707154.1 | 25.29 | 4.40 | 86.51 | 3.91 | 105.27 | 17.71 |
| AD-1707144.1 | 26.58 | 6.16 | 94.66 | 17.37 | 101.45 | 9.43 |
| AD-1707134.1 | 28.54 | 6.01 | 99.26 | 11.19 | 89.22 | 17.73 |
| AD-1707159.1 | 28.79 | 5.65 | 107.12 | 15.73 | 105.46 | 10.57 |
| AD-1707149.1 | 29.32 | 2.79 | 155.91 | 29.51 | 94.83 | 22.67 |
| AD-1707137.1 | 29.64 | 2.96 | 105.39 | 14.31 | 104.18 | 20.24 |
| AD-1708957.1 | 30.15 | 5.52 | 88.33 | 6.86 | 98.55 | 13.20 |
| AD-1707160.1 | 32.76 | 3.02 | 88.95 | 7.30 | 116.76 | 17.01 |
| AD-1711769.1 | 32.88 | 5.54 | 83.54 | 10.19 | 93.62 | 9.98 |
| AD-1711767.1 | 33.68 | 3.93 | 92.83 | 21.12 | 106.68 | 6.50 |
| AD-1711770.1 | 34.57 | 6.07 | 99.82 | 17.88 | 93.32 | 18.62 |
| AD-1707169.1 | 36.98 | 4.18 | 110.99 | 33.26 | 105.09 | 14.13 |
| AD-1707210.1 | 37.13 | 8.07 | 95.18 | 12.01 | 100.39 | 16.02 |
| AD-1711765.1 | 37.53 | 15.27 | 100.34 | 14.43 | 100.63 | 30.48 |
| AD-1707173.1 | 37.95 | 3.50 | 96.79 | 15.17 | 101.14 | 6.92 |
| AD-1707162.1 | 38.24 | 3.82 | 116.22 | 44.16 | 107.92 | 20.94 |
| AD-1708974.1 | 38.68 | 2.37 | 109.51 | 33.43 | 100.84 | 3.83 |
| AD-1707140.1 | 38.77 | 6.98 | 87.96 | 19.28 | 102.71 | 19.68 |
| AD-1707145.1 | 38.82 | 7.25 | 82.00 | 14.95 | 112.74 | 34.49 |
| AD-1708971.1 | 38.93 | 6.26 | 104.04 | 20.22 | 94.40 | 15.26 |
| AD-1707166.1 | 39.32 | 7.06 | 101.96 | 7.66 | 98.75 | 7.43 |
| AD-1707151.1 | 40.38 | 6.23 | 97.29 | 17.49 | 106.17 | 15.07 |
| AD-1708963.1 | 40.88 | 2.50 | 118.93 | 18.80 | 108.47 | 11.31 |
| AD-1707153.1 | 40.91 | 6.24 | 92.88 | 15.02 | 97.58 | 7.63 |
| AD-1707157.1 | 44.06 | 14.33 | 97.51 | 12.37 | 98.71 | 6.68 |
| AD-1707158.1 | 44.79 | 7.72 | 99.01 | 16.41 | 111.74 | 9.29 |
| AD-1708990.1 | 46.04 | 5.68 | 96.56 | 15.31 | 110.10 | 7.50 |
| AD-1707197.1 | 46.35 | 4.78 | 99.28 | 9.06 | 103.29 | 2.99 |
| AD-1707168.1 | 46.52 | 7.25 | 114.85 | 40.01 | 121.67 | 21.33 |
| AD-1707164.1 | 46.52 | 4.87 | 107.61 | 4.25 | 105.06 | 9.80 |
| AD-1707142.1 | 48.03 | 9.69 | 88.73 | 20.26 | 97.14 | 19.70 |
| AD-1707147.1 | 48.39 | 9.65 | 102.36 | 10.85 | 106.88 | 15.92 |
| AD-1707133.1 | 48.55 | 7.85 | 90.89 | 12.15 | 94.27 | 15.48 |
| AD-1707156.1 | 50.21 | 9.50 | 87.48 | 10.24 | 100.76 | 13.47 |
| AD-1707150.1 | 50.56 | 5.47 | 128.77 | 29.09 | 106.27 | 11.80 |
| AD-1711768.1 | 51.31 | 6.81 | 94.55 | 5.18 | 86.83 | 16.67 |
| AD-1707177.1 | 52.17 | 7.40 | 97.31 | 19.25 | 93.24 | 6.70 |
| AD-1707176.1 | 52.38 | 5.97 | 103.31 | 15.62 | 123.43 | 31.50 |
| AD-1707209.1 | 54.24 | 10.92 | 90.52 | 15.49 | 93.77 | 7.34 |
| AD-1707135.1 | 55.06 | 6.19 | 97.90 | 12.98 | 95.86 | 18.67 |
| AD-1707201.1 | 56.43 | 9.50 | 141.33 | 27.95 | 111.61 | 7.09 |
| AD-1707141.1 | 56.83 | 21.84 | 106.75 | 16.22 | 96.91 | 17.42 |
| AD-1707139.1 | 56.98 | 2.61 | 108.61 | 19.57 | 96.36 | 9.51 |
| AD-1707163.1 | 57.43 | 9.97 | 102.98 | 6.88 | 110.92 | 8.77 |
| AD-1707211.1 | 58.08 | 16.77 | 100.46 | 12.93 | 89.06 | 15.85 |
| AD-1707171.1 | 59.68 | 7.29 | 88.87 | 10.18 | 96.17 | 14.09 |
| AD-1711764.1 | 61.91 | 4.80 | 88.61 | 13.52 | 94.06 | 7.03 |
| AD-1709201.1 | 62.48 | 5.31 | 83.62 | 13.75 | 89.35 | 9.59 |
| AD-1707167.1 | 64.49 | 6.21 | 88.47 | 6.99 | 93.88 | 9.17 |
| AD-1707155.1 | 64.64 | 13.86 | 107.37 | 28.96 | 106.86 | 12.33 |
| AD-1707202.1 | 65.35 | 7.10 | 95.62 | 18.04 | 98.52 | 13.35 |
| AD-1707203.1 | 66.47 | 8.06 | 105.42 | 26.74 | 102.48 | 8.85 |
| AD-1708977.1 | 67.97 | 6.89 | 111.17 | 12.98 | 112.14 | 9.94 |
| AD-1707178.1 | 68.77 | 23.43 | 115.97 | 35.56 | 85.13 | 14.23 |
| AD-1707200.1 | 69.32 | 10.45 | 105.72 | 23.83 | 104.89 | 7.43 |
| AD-1707138.1 | 74.31 | 12.00 | 107.37 | 12.17 | 108.82 | 8.30 |
| AD-1707207.1 | 76.91 | 13.93 | 119.04 | 36.69 | 105.00 | 9.19 |
| AD-1707190.1 | 77.90 | 11.71 | 92.83 | 14.82 | 93.57 | 9.45 |
| AD-1708984.1 | 78.01 | 8.72 | 95.54 | 14.71 | 103.87 | 15.45 |
| AD-1707148.1 | 78.60 | 9.21 | 105.45 | 9.27 | 106.10 | 20.83 |
| AD-1707172.1 | 79.74 | 8.47 | 106.69 | 11.16 | 99.53 | 16.18 |
| AD-1708986.1 | 80.65 | 10.97 | 121.14 | 24.43 | 108.29 | 8.71 |
| AD-1707165.1 | 82.20 | 12.26 | 128.70 | 34.94 | 101.82 | 15.99 |
| AD-1708962.1 | 84.11 | 7.41 | 91.81 | 13.52 | 81.71 | 30.08 |
| AD-1707204.1 | 84.21 | 9.53 | 104.10 | 25.70 | 98.83 | 16.90 |
| AD-1711772.1 | 84.59 | 8.95 | 91.65 | 21.63 | 102.48 | 22.65 |
| AD-1711766.1 | 87.33 | 12.72 | 93.37 | 15.24 | 109.16 | 2.18 |
| AD-1709022.1 | 87.35 | 12.09 | 89.48 | 15.28 | 102.93 | 16.68 |
| AD-1709028.1 | 88.42 | 9.93 | 93.89 | 24.08 | 104.19 | 7.73 |
| AD-1707179.1 | 89.17 | 13.08 | 100.09 | 8.41 | 105.13 | 8.34 |
| AD-1707205.1 | 92.52 | 12.36 | 108.95 | 4.96 | 112.68 | 11.95 |
| AD-1707180.1 | 93.48 | 21.70 | 98.26 | 9.31 | 105.78 | 14.35 |
| AD-1707174.1 | 93.87 | 10.14 | 95.00 | 16.23 | 109.95 | 15.51 |
| AD-1707208.1 | 94.27 | 14.12 | 117.41 | 38.11 | 110.38 | 9.20 |
| AD-1708976.1 | 94.71 | 18.83 | 101.36 | 7.68 | 107.96 | 12.07 |
| AD-1709021.1 | 97.52 | 17.75 | 92.57 | 13.43 | 99.20 | 6.38 |
| AD-1707189.1 | 99.48 | 6.62 | 96.95 | 9.64 | 97.68 | 11.39 |
| AD-1707206.1 | 100.82 | 16.60 | 96.42 | 11.21 | 106.77 | 24.93 |
| AD-1711771.1 | 104.52 | 3.03 | 88.98 | 15.82 | 102.81 | 12.47 |
| AD-1709026.1 | 116.40 | 6.19 | 101.82 | 20.63 | 112.17 | 12.49 |
| AD-1707182.1 | 118.95 | 22.62 | 116.54 | 37.28 | 114.08 | 5.65 |
| AD-1709009.1 | 119.75 | 15.80 | 122.88 | 22.76 | 108.96 | 16.73 |
| AD-1709032.1 | 10.65 | 1.46 | 72.91 | 9.78 | 87.30 | 6.27 |
| AD-1709044.1 | 10.97 | 0.72 | 93.48 | 14.31 | 112.97 | 13.33 |
| AD-1707224.1 | 12.74 | 2.75 | 71.54 | 13.48 | 91.74 | 19.65 |
| AD-1707306.1 | 13.25 | 1.84 | 78.47 | 12.33 | 103.70 | 13.65 |
| AD-1707225.1 | 13.42 | 2.08 | 75.80 | 6.57 | 107.46 | 18.05 |
| AD-1707223.1 | 13.58 | 4.79 | 79.36 | 12.93 | 108.17 | 24.63 |
| AD-1707305.1 | 13.79 | 0.84 | 97.69 | 13.40 | 103.89 | 16.61 |
| AD-1711773.1 | 13.84 | 1.32 | 90.16 | 16.57 | 134.96 | 30.18 |
| AD-1707214.1 | 14.01 | 4.99 | 80.78 | 9.25 | 102.66 | 21.99 |
| AD-1709038.1 | 14.03 | 1.38 | 100.99 | 17.22 | 138.67 | 38.28 |
| AD-1707317.1 | 14.12 | 1.62 | 84.03 | 15.86 | 107.44 | 6.76 |
| AD-1707216.1 | 14.52 | 2.86 | 86.31 | 10.19 | 85.93 | 0.00 |
| AD-1707318.1 | 14.74 | 1.94 | 69.40 | 5.08 | 127.81 | 35.42 |
| AD-1707304.1 | 15.23 | 2.90 | 97.01 | 7.85 | 152.32 | 32.19 |
| AD-1707313.1 | 15.77 | 3.15 | 80.19 | 14.57 | 98.62 | 21.63 |
| AD-1707221.1 | 16.00 | 2.73 | 83.56 | 12.11 | 104.63 | 25.69 |
| AD-1707213.1 | 17.50 | 1.12 | 91.79 | 13.61 | 110.12 | 14.97 |
| AD-1707218.1 | 18.01 | 2.72 | 95.39 | 8.58 | 137.53 | 31.38 |
| AD-1709130.1 | 18.28 | 2.33 | 73.33 | 9.92 | 95.68 | 6.54 |
| AD-1711774.1 | 18.77 | 16.50 | 98.28 | 26.33 | 146.32 | 49.75 |
| AD-1707303.1 | 18.77 | 2.61 | 86.31 | 8.27 | 118.33 | 22.15 |
| AD-1707290.1 | 19.51 | 2.26 | 97.82 | 7.63 | 114.46 | 14.53 |
| AD-1709034.1 | 19.87 | 8.67 | 95.49 | 13.97 | 134.79 | 30.19 |
| AD-1707289.1 | 21.00 | 5.23 | 94.06 | 5.36 | 111.31 | 11.21 |
| AD-1707280.1 | 21.21 | 2.95 | 86.67 | 9.47 | 96.46 | 8.51 |
| AD-1707212.1 | 22.71 | 3.39 | 94.41 | 5.67 | 120.86 | 31.47 |
| AD-1707288.1 | 22.72 | 4.43 | 93.00 | 14.56 | 113.10 | 14.44 |
| AD-1707291.1 | 22.84 | 3.29 | 105.07 | 22.57 | 105.16 | 16.77 |
| AD-1707314.1 | 23.13 | 3.99 | 98.59 | 23.48 | 106.37 | 23.07 |
| AD-1707308.1 | 23.74 | 4.68 | 95.43 | 13.97 | 113.65 | 28.74 |
| AD-1707217.1 | 25.94 | 18.18 | 80.34 | 11.48 | 105.73 | 13.57 |
| AD-1707287.1 | 25.99 | 6.84 | 92.78 | 9.70 | 108.94 | 18.00 |
| AD-1707299.1 | 26.27 | 3.08 | 97.87 | 5.04 | 123.11 | 10.32 |
| AD-1707292.1 | 27.32 | 2.25 | 99.62 | 13.65 | 103.35 | 36.19 |
| AD-1709124.1 | 27.62 | 2.53 | 93.80 | 21.22 | 117.31 | 37.59 |
| AD-1707312.1 | 28.79 | 3.24 | 101.19 | 13.56 | 125.78 | 26.36 |
| AD-1707311.1 | 29.58 | 5.31 | 102.79 | 19.49 | 108.80 | 18.25 |
| AD-1707231.1 | 29.70 | 3.41 | 110.87 | 12.10 | 122.78 | 6.80 |
| AD-1707315.1 | 29.82 | 4.37 | 94.22 | 15.09 | 117.40 | 27.65 |
| AD-1709041.1 | 30.64 | 5.89 | 112.79 | 34.30 | 123.67 | 39.64 |
| AD-1709090.1 | 30.91 | 4.76 | 96.67 | 16.65 | 106.46 | 17.17 |
| AD-1707337.1 | 31.57 | 5.82 | 93.58 | 16.80 | 108.70 | 17.59 |
| AD-1707281.1 | 33.35 | 3.66 | 101.45 | 3.78 | 107.40 | 22.02 |
| AD-1707284.1 | 33.41 | 3.61 | 98.04 | 21.19 | 117.03 | 25.08 |
| AD-1707310.1 | 35.62 | 1.48 | 118.56 | 40.26 | 107.58 | 17.75 |
| AD-1709102.1 | 35.85 | 4.29 | 107.66 | 21.90 | 124.11 | 45.38 |
| AD-1707307.1 | 35.88 | 8.73 | 96.17 | 12.20 | 109.75 | 10.94 |
| AD-1707226.1 | 36.27 | 4.27 | 107.71 | 10.16 | 111.11 | 11.64 |
| AD-1707302.1 | 36.97 | 7.05 | 91.71 | 19.52 | 113.79 | 16.00 |
| AD-1709126.1 | 37.18 | 7.60 | 101.43 | 19.25 | 124.72 | 16.78 |
| AD-1707228.1 | 38.56 | 18.34 | 98.73 | 17.99 | 112.83 | 33.73 |
| AD-1707278.1 | 41.71 | 3.76 | 106.79 | 12.12 | 120.88 | 13.90 |
| AD-1709104.1 | 42.57 | 6.08 | 98.63 | 15.39 | 107.51 | 18.98 |
| AD-1707227.1 | 43.56 | 5.85 | 105.34 | 7.24 | 106.96 | 21.11 |
| AD-1707279.1 | 43.64 | 6.62 | 95.09 | 24.45 | 98.32 | 22.69 |
| AD-1709122.1 | 45.93 | 9.47 | 121.35 | 21.87 | 110.30 | 15.15 |
| AD-1709052.1 | 48.00 | 6.56 | 104.18 | 17.40 | 141.43 | 55.52 |
| AD-1707286.1 | 49.25 | 9.69 | 96.94 | 11.77 | 107.88 | 8.01 |
| AD-1707232.1 | 49.71 | 4.79 | 103.13 | 6.35 | 101.42 | 20.85 |
| AD-1707239.1 | 51.17 | 9.25 | 94.08 | 19.53 | 111.03 | 12.43 |
| AD-1709100.1 | 51.61 | 16.14 | 106.55 | 9.21 | 138.74 | 28.67 |
| AD-1707293.1 | 52.59 | 13.82 | 118.95 | 30.71 | 102.52 | 20.23 |
| AD-1707338.1 | 52.76 | 8.61 | 101.81 | 10.45 | 115.79 | 26.48 |
| AD-1707283.1 | 53.75 | 5.20 | 120.04 | 14.87 | 136.81 | 54.63 |
| AD-1707285.1 | 54.11 | 6.28 | 108.86 | 7.48 | 113.13 | 12.98 |
| AD-1707301.1 | 55.03 | 8.84 | 90.63 | 17.10 | 107.24 | 27.55 |
| AD-1707309.1 | 56.19 | 6.30 | 97.08 | 9.15 | 105.76 | 21.42 |
| AD-1707240.1 | 58.92 | 8.28 | 96.05 | 10.01 | 93.42 | 16.45 |
| AD-1707316.1 | 59.31 | 17.56 | 109.46 | 32.98 | 157.17 | 69.02 |
| AD-1707277.1 | 60.63 | 17.08 | 100.69 | 11.68 | 115.49 | 9.64 |
| AD-1709106.1 | 61.42 | 6.05 | 98.37 | 14.84 | 129.70 | 28.96 |
| AD-1709120.1 | 62.12 | 11.73 | 96.87 | 22.29 | 120.52 | 32.78 |
| AD-1707276.1 | 62.36 | 7.14 | 101.51 | 20.17 | 100.78 | 19.23 |
| AD-1709148.1 | 62.57 | 15.47 | 94.00 | 3.55 | 95.21 | 2.66 |
| AD-1707275.1 | 62.62 | 6.51 | 93.58 | 14.35 | 99.46 | 19.67 |
| AD-1709111.1 | 63.90 | 15.14 | 95.56 | 9.44 | 120.37 | 39.53 |
| AD-1709097.1 | 69.40 | 7.55 | 103.39 | 27.37 | 128.13 | 10.58 |
| AD-1707282.1 | 69.63 | 7.45 | 99.24 | 17.79 | 124.74 | 38.28 |
| AD-1707234.1 | 70.71 | 9.87 | 114.33 | 37.40 | 110.04 | 19.59 |
| AD-1707230.1 | 74.46 | 12.47 | 95.56 | 8.67 | 115.51 | 17.32 |
| AD-1709098.1 | 79.18 | 4.31 | 104.27 | 10.48 | 116.87 | 26.13 |
| AD-1707241.1 | 79.49 | 9.37 | 111.57 | 20.66 | 120.03 | 23.30 |
| AD-1707236.1 | 85.92 | 13.55 | 109.82 | 23.40 | 131.32 | 45.71 |
| AD-1709128.1 | 86.19 | 11.09 | 117.37 | 14.30 | 114.43 | 20.56 |
| AD-1707242.1 | 87.51 | 6.96 | 97.20 | 14.18 | 110.78 | 22.01 |
| AD-1707233.1 | 91.33 | 10.63 | 105.56 | 19.90 | 95.12 | 23.29 |
| AD-1707237.1 | 91.92 | 10.46 | 106.06 | 19.52 | 113.76 | 36.13 |
| AD-1707229.1 | 92.04 | 4.24 | 101.96 | 21.38 | 130.00 | 26.36 |
| AD-1709056.1 | 94.72 | 20.30 | 109.17 | 32.34 | 119.96 | 16.65 |
| AD-1709031.1 | 107.23 | 18.65 | 105.11 | 9.56 | 115.47 | 29.46 |
| AD-1707636.1 | 10.58 | 3.09 | 73.37 | 13.26 | 101.03 | 16.01 |
| AD-1711779.1 | 12.37 | 2.63 | 71.60 | 15.39 | 110.73 | 23.21 |
| AD-1707639.1 | 14.43 | 3.04 | 76.92 | 14.30 | 99.84 | 12.54 |
| AD-1707641.1 | 14.56 | 1.31 | 72.69 | 13.14 | 87.79 | 19.99 |
| AD-1707640.1 | 15.36 | 5.50 | 73.67 | 8.05 | 84.94 | 24.69 |
| AD-1707638.1 | 15.84 | 2.03 | 74.50 | 10.42 | 102.75 | 14.50 |
| AD-1707637.1 | 16.00 | 1.31 | 84.11 | 20.33 | 120.69 | 21.72 |
| AD-1707340.1 | 18.02 | 4.57 | 71.49 | 7.53 | 86.85 | 11.99 |
| AD-1707632.1 | 19.56 | 3.37 | 101.07 | 23.16 | 99.62 | 10.24 |
| AD-1711775.1 | 20.20 | 2.65 | 91.97 | 18.77 | 103.33 | 4.55 |
| AD-1707339.1 | 21.62 | 5.49 | 91.29 | 16.30 | 107.99 | 14.63 |
| AD-1707634.1 | 23.26 | 5.19 | 94.39 | 10.58 | 110.43 | 6.43 |
| AD-1709151.1 | 24.09 | 2.26 | 95.46 | 13.85 | 85.84 | 17.45 |
| AD-1709442.1 | 24.19 | 1.70 | 99.99 | 21.91 | 106.91 | 7.38 |
| AD-1707630.1 | 24.82 | 2.27 | 82.71 | 18.99 | 100.07 | 9.51 |
| AD-1709310.1 | 25.64 | 4.25 | 113.85 | 44.32 | 117.47 | 5.21 |
| AD-1707605.1 | 25.97 | 3.89 | 94.46 | 16.93 | 109.86 | 6.59 |
| AD-1709372.1 | 27.94 | 2.72 | 115.20 | 28.81 | 94.89 | 9.83 |
| AD-1707631.1 | 28.93 | 4.48 | 100.66 | 29.44 | 98.29 | 12.45 |
| AD-1711776.1 | 29.75 | 7.50 | 96.97 | 16.01 | 79.21 | 26.10 |
| AD-1707633.1 | 30.49 | 6.28 | 103.63 | 27.18 | 99.15 | 10.39 |
| AD-1709301.1 | 31.17 | 2.49 | 100.61 | 18.34 | 106.10 | 9.33 |
| AD-1707466.1 | 33.17 | 3.58 | 80.17 | 10.16 | 91.78 | 17.83 |
| AD-1709410.1 | 33.26 | 11.51 | 109.37 | 45.97 | 89.98 | 28.25 |
| AD-1707629.1 | 34.41 | 10.14 | 100.46 | 13.51 | 108.44 | 26.17 |
| AD-1707388.1 | 34.54 | 7.23 | 95.22 | 14.50 | 102.57 | 10.06 |
| AD-1707341.1 | 35.41 | 11.95 | 89.30 | 5.48 | 100.63 | 9.92 |
| AD-1707472.1 | 36.07 | 7.86 | 79.99 | 12.62 | 97.28 | 14.58 |
| AD-1707470.1 | 37.22 | 3.26 | 80.96 | 13.21 | 106.70 | 7.03 |
| AD-1709300.1 | 37.80 | 2.04 | 133.54 | 24.47 | 107.60 | 9.91 |
| AD-1707606.1 | 37.97 | 8.54 | 103.68 | 30.81 | 99.47 | 10.48 |
| AD-1709434.1 | 38.13 | 5.93 | 93.06 | 10.01 | 92.27 | 6.32 |
| AD-1707479.1 | 38.62 | 3.53 | 100.45 | 19.44 | 104.42 | 10.85 |
| AD-1707474.1 | 40.76 | 12.33 | 91.91 | 7.21 | 92.10 | 6.30 |
| AD-1707478.1 | 40.81 | 7.35 | 92.42 | 19.60 | 92.52 | 18.25 |
| AD-1709433.1 | 40.81 | 2.17 | 93.17 | 12.95 | 107.44 | 15.95 |
| AD-1707342.1 | 41.92 | 3.25 | 95.88 | 23.23 | 99.12 | 7.85 |
| AD-1707481.1 | 43.58 | 6.80 | 82.30 | 6.97 | 84.65 | 18.42 |
| AD-1707577.1 | 43.88 | 4.91 | 85.72 | 6.14 | 95.04 | 14.26 |
| AD-1707556.1 | 44.01 | 5.20 | 101.93 | 28.43 | 101.74 | 10.99 |
| AD-1711777.1 | 45.14 | 11.21 | 114.45 | 10.62 | 96.73 | 20.52 |
| AD-1709443.1 | 45.22 | 0.83 | 100.07 | 11.26 | 97.92 | 7.67 |
| AD-1707486.1 | 45.51 | 9.49 | 100.63 | 5.39 | 100.14 | 11.85 |
| AD-1707628.1 | 46.62 | 6.73 | 113.39 | 21.12 | 93.74 | 13.75 |
| AD-1707480.1 | 46.88 | 6.69 | 102.09 | 18.77 | 110.14 | 13.44 |
| AD-1709411.1 | 47.31 | 13.14 | 87.52 | 8.56 | 91.88 | 24.45 |
| AD-1711778.1 | 48.28 | 11.80 | 111.20 | 13.56 | 94.81 | 18.75 |
| AD-1709237.1 | 48.28 | 6.38 | 94.22 | 8.15 | 96.80 | 12.70 |
| AD-1707483.1 | 48.39 | 4.85 | 99.57 | 17.10 | 94.96 | 21.55 |
| AD-1709304.1 | 50.75 | 12.20 | 99.78 | 16.16 | 92.66 | 11.59 |
| AD-1707578.1 | 51.22 | 9.70 | 105.42 | 11.28 | 98.33 | 5.94 |
| AD-1707626.1 | 51.23 | 8.00 | 93.89 | 16.64 | 107.20 | 11.75 |
| AD-1707553.1 | 51.76 | 10.35 | 97.28 | 9.61 | 95.98 | 19.82 |
| AD-1709317.1 | 53.03 | 12.07 | 95.68 | 17.98 | 106.76 | 17.19 |
| AD-1707627.1 | 53.41 | 17.00 | 90.59 | 17.84 | 98.25 | 18.90 |
| AD-1709429.1 | 53.84 | 4.92 | 98.91 | 15.37 | 103.18 | 17.21 |
| AD-1707469.1 | 54.22 | 8.62 | 96.86 | 15.86 | 105.03 | 6.45 |
| AD-1707555.1 | 54.36 | 14.24 | 95.46 | 9.45 | 101.46 | 22.99 |
| AD-1709502.1 | 54.67 | 11.47 | 97.24 | 11.50 | 96.65 | 11.03 |
| AD-1707467.1 | 55.71 | 8.17 | 105.11 | 18.31 | 108.78 | 10.76 |
| AD-1707473.1 | 57.10 | 9.09 | 85.76 | 12.49 | 101.37 | 17.12 |
| AD-1707557.1 | 58.60 | 9.88 | 96.92 | 24.28 | 97.24 | 15.17 |
| AD-1707625.1 | 59.10 | 4.85 | 107.25 | 29.88 | 114.62 | 8.75 |
| AD-1707482.1 | 59.61 | 7.43 | 105.12 | 9.08 | 107.28 | 12.66 |
| AD-1707471.1 | 60.25 | 8.69 | 115.63 | 30.61 | 110.02 | 25.57 |
| AD-1707468.1 | 60.62 | 11.06 | 98.79 | 8.96 | 98.07 | 21.08 |
| AD-1709307.1 | 60.77 | 11.96 | 104.13 | 18.31 | 99.16 | 16.21 |
| AD-1707416.1 | 62.39 | 10.60 | 106.20 | 29.09 | 108.74 | 6.92 |
| AD-1707476.1 | 62.69 | 6.78 | 98.18 | 21.33 | 114.57 | 25.32 |
| AD-1707477.1 | 63.06 | 14.52 | 106.74 | 20.36 | 106.68 | 14.21 |
| AD-1707475.1 | 65.11 | 5.23 | 98.37 | 14.42 | 105.46 | 7.86 |
| AD-1709254.1 | 66.79 | 23.87 | 101.35 | 11.41 | 89.71 | 18.70 |
| AD-1707390.1 | 67.23 | 7.60 | 100.09 | 13.09 | 95.79 | 8.38 |
| AD-1709298.1 | 68.03 | 10.97 | 96.34 | 17.17 | 108.50 | 15.76 |
| AD-1709390.1 | 68.34 | 8.57 | 105.65 | 25.15 | 99.82 | 10.51 |
| AD-1707484.1 | 69.92 | 9.74 | 97.85 | 20.58 | 99.48 | 13.58 |
| AD-1709314.1 | 72.02 | 3.76 | 122.26 | 28.02 | 109.58 | 16.56 |
| AD-1709431.1 | 73.69 | 13.99 | 115.10 | 20.10 | 103.94 | 18.67 |
| AD-1707554.1 | 74.14 | 16.74 | 103.41 | 15.16 | 99.75 | 11.58 |
| AD-1707412.1 | 78.12 | 15.95 | 135.10 | 34.55 | 107.09 | 20.15 |
| AD-1707624.1 | 81.73 | 10.74 | 121.46 | 18.90 | 111.10 | 15.87 |
| AD-1707623.1 | 84.10 | 14.14 | 117.90 | 34.70 | 101.07 | 11.24 |
| AD-1707485.1 | 85.82 | 5.18 | 96.24 | 6.20 | 108.58 | 7.26 |
| AD-1707418.1 | 89.78 | 16.64 | 90.19 | 15.94 | 96.15 | 12.68 |
| AD-1709315.1 | 92.79 | 10.37 | 93.51 | 7.08 | 103.41 | 13.56 |
| AD-1707411.1 | 95.22 | 14.65 | 90.23 | 10.17 | 99.59 | 20.10 |
| AD-1709239.1 | 95.51 | 10.13 | 94.42 | 8.85 | 94.47 | 10.55 |
| AD-1707415.1 | 96.35 | 27.83 | 87.14 | 14.90 | 101.20 | 19.14 |
| AD-1709253.1 | 99.77 | 27.82 | 101.30 | 20.97 | 100.35 | 31.79 |
| AD-1707417.1 | 104.79 | 17.20 | 115.55 | 22.60 | 107.73 | 8.99 |

TABLE 21B
Single Dose Screen for dsRNA agents targeting INHBE in PHH cells (% average mRNA remaining).

| Duplex | Primary Human Hepatocytes Transfection | | | | | | Primary Human Hepatocytes Free Uptake | | | | | | | |
|---|---|---|---|---|---|---|---|---|---|---|---|---|---|---|
| Name | 10 nM | StDev | 1 nM | StDev | 0.1 nM | StDev | 250 nM | StDev | 100 nM | StDev | 10 nM | StDev | 1 nM | StDev |
| AD-1708473 | 3.84 | 1.35 | 4.83 | 0.78 | 11.90 | 5.80 | 9.05 | 2.26 | 7.91 | 2.05 | 20.30 | 4.14 | 26.88 | 9.48 |
| AD-1707061 | 4.14 | 0.80 | 7.73 | 1.54 | 10.01 | 4.27 | 5.05 | 2.70 | 9.15 | 1.74 | 19.29 | 6.44 | 22.54 | 5.46 |
| AD-1708478 | 4.21 | 0.86 | 8.74 | 1.95 | 12.12 | 2.57 | 15.32 | 3.35 | 15.49 | 4.78 | 32.44 | 12.86 | 47.21 | 3.08 |
| AD-1706584 | 6.01 | 1.00 | 12.11 | 2.32 | 20.47 | 1.86 | 15.62 | 6.96 | 12.92 | 3.51 | 31.81 | 9.56 | 39.52 | 13.24 |
| AD-1707306 | 5.41 | 0.99 | 8.64 | 1.68 | 15.40 | 3.91 | 14.67 | 4.08 | 12.03 | 5.26 | 31.22 | 4.85 | 29.94 | 12.32 |
| AD-1711744 | 7.34 | 0.93 | 22.17 | 1.93 | 24.84 | 6.82 | 16.09 | 6.18 | 18.31 | 7.16 | 46.93 | 10.77 | 21.15 | 6.60 |
| AD-1707639 | 7.39 | 0.40 | 15.74 | 1.72 | 25.11 | 1.15 | 20.36 | 7.67 | 22.33 | 6.58 | 33.27 | 5.33 | 35.71 | 13.43 |
| AD-1707641 | 6.30 | 1.05 | 11.48 | 0.98 | 13.72 | 2.64 | 14.35 | 5.72 | 16.48 | 6.07 | 27.41 | 4.58 | 37.17 | 12.82 |
| AD-1707640 | 14.25 | 5.27 | 9.89 | 2.61 | 11.62 | 2.53 | 19.98 | 9.23 | 9.86 | 1.84 | 29.19 | 8.23 | 46.42 | 16.24 |
| AD-1706583 | 9.59 | 2.03 | 11.01 | 2.55 | 33.23 | 7.79 | 32.08 | 9.27 | 34.18 | 1.07 | 44.93 | 7.59 | 47.65 | 14.84 |
| AD-1708956 | 8.29 | 1.74 | 7.69 | 1.78 | 20.17 | 2.12 | 27.77 | 10.81 | 21.34 | 8.65 | 51.13 | 12.95 | 62.63 | 10.70 |
| AD-1706591 | 17.87 | 2.33 | 21.38 | 2.89 | 33.74 | 2.09 | 35.01 | 8.34 | 43.04 | 5.73 | 55.08 | 8.42 | 49.40 | 1.88 |
| AD-1706593 | 16.39 | 2.72 | 21.56 | 5.13 | 38.39 | 4.68 | 38.38 | 2.01 | 40.78 | 12.66 | 67.80 | 3.57 | 57.75 | 2.38 |
| AD-1707136 | 9.73 | 2.61 | 12.76 | 1.74 | 18.58 | 4.48 | 21.70 | 6.52 | 26.72 | 8.13 | 49.51 | 9.16 | 62.04 | 21.31 |
| AD-1706587 | 19.27 | 2.95 | 29.03 | 3.31 | 42.92 | 2.35 | 37.95 | 9.76 | 48.29 | 7.11 | 63.48 | 11.56 | 91.09 | 9.80 |
| AD-1706547 | 9.05 | 1.63 | 19.91 | 2.05 | 26.11 | 6.44 | 44.96 | 7.86 | 39.42 | 6.77 | 60.57 | 6.79 | 51.76 | 15.33 |
| AD-1708399 | 16.90 | 6.46 | 26.04 | 3.67 | 32.84 | 8.56 | 27.69 | 8.64 | 31.00 | 7.18 | 40.88 | 11.44 | 65.34 | 24.97 |
| AD-1707340 | 12.13 | 0.79 | 18.29 | 2.31 | 35.44 | 5.97 | 51.01 | 14.05 | 57.39 | 11.96 | 66.47 | 11.16 | 81.96 | 6.06 |
| AD-1706662 | 10.58 | 2.12 | 14.50 | 1.46 | 21.49 | 2.39 | 38.97 | 3.56 | 46.03 | 9.20 | 51.78 | 16.38 | 75.97 | 5.93 |
| AD-1709130 | 20.00 | 2.26 | 22.86 | 3.41 | 35.13 | 5.88 | 33.52 | 8.58 | 31.04 | 5.07 | 48.54 | 15.67 | 85.19 | 20.45 |
| AD-1708882 | 14.97 | 1.54 | 17.39 | 3.37 | 41.21 | 1.66 | 33.36 | 6.74 | 51.38 | 5.73 | 43.23 | 14.27 | 93.31 | 10.28 |
| AD-1707087 | 10.79 | 2.18 | 20.93 | 4.44 | 27.23 | 4.01 | 33.09 | 7.91 | 45.27 | 4.15 | 61.83 | 8.84 | 92.73 | 15.04 |
| AD-1707632 | 14.96 | 3.95 | 27.48 | 2.83 | 34.46 | 5.77 | 60.90 | 17.76 | 45.59 | 14.85 | 62.59 | 2.82 | 83.12 | 21.68 |
| AD-1709034 | 14.35 | 3.38 | 23.39 | 2.66 | 32.18 | 8.41 | 42.08 | 9.32 | 44.37 | 7.57 | 72.83 | 4.37 | 92.28 | 2.99 |
| AD-1706761 | 9.18 | 3.40 | 17.68 | 4.49 | 23.93 | 4.88 | 34.19 | 6.86 | 41.28 | 7.96 | 54.90 | 3.62 | 86.71 | 21.91 |
| AD-1711775 | 29.82 | 4.53 | 38.15 | 3.94 | 51.68 | 4.29 | 76.87 | 14.21 | 89.53 | 9.82 | 94.63 | 6.00 | 124.76 | 12.67 |
| AD-1706758 | 29.57 | 6.34 | 33.96 | 2.09 | 53.10 | 9.12 | 59.66 | 3.42 | 65.91 | 9.15 | 65.69 | 12.66 | 105.66 | 22.32 |
| AD-1707339 | 28.32 | 2.83 | 31.07 | 1.55 | 52.58 | 4.24 | 59.15 | 8.31 | 73.87 | 2.72 | 86.02 | 5.27 | 100.65 | 6.19 |
| AD-1708368 | 24.61 | 5.44 | 37.31 | 3.95 | 60.77 | 6.60 | 79.25 | 15.90 | 67.90 | 12.41 | 90.06 | 6.70 | 131.88 | 7.90 |
| AD-1706762 | 11.26 | 2.98 | 25.09 | 2.72 | 35.72 | 3.49 | 46.85 | 13.20 | 50.92 | 17.34 | 84.37 | 11.82 | 94.03 | 4.93 |
| AD-1707291 | 8.89 | 0.54 | 16.30 | 2.83 | 28.25 | 7.47 | 42.60 | 7.79 | 37.31 | 5.08 | 68.87 | 7.93 | 72.32 | 15.94 |
| AD-1707314 | 20.85 | 2.68 | 25.87 | 2.99 | 42.31 | 12.16 | 56.47 | 12.93 | 42.72 | 5.67 | 66.58 | 13.04 | 88.09 | 1.99 |
| AD-1706640 | 16.91 | 3.11 | 36.66 | 3.88 | 40.86 | 10.18 | 28.39 | 6.96 | 46.43 | 6.76 | 78.03 | 17.64 | 133.63 | 32.82 |
| AD-1707054 | 14.13 | 2.53 | 22.61 | 2.39 | 34.17 | 3.97 | 55.10 | 7.84 | 59.59 | 7.96 | 87.74 | 3.87 | 119.92 | 16.53 |
| AD-1706641 | 24.33 | 3.32 | 32.23 | 5.92 | 45.48 | 8.35 | 61.06 | 5.81 | 66.77 | 17.98 | 83.39 | 19.40 | 107.19 | 7.61 |
| AD-1707308 | 15.25 | 2.92 | 27.67 | 2.72 | 36.27 | 7.33 | 51.25 | 8.34 | 43.23 | 11.57 | 88.07 | 18.70 | 103.21 | 9.29 |
| AD-1709151 | 32.08 | 6.47 | 44.97 | 9.45 | 50.34 | 12.76 | 90.75 | 8.27 | 82.92 | 14.60 | 115.33 | 3.62 | 122.19 | 13.92 |
| AD-1706656 | 8.91 | 1.04 | 21.35 | 4.03 | 31.89 | 3.90 | 43.20 | 8.76 | 58.20 | 16.02 | 89.89 | 23.74 | 118.47 | 20.90 |
| AD-1706651 | 17.38 | 2.79 | 24.64 | 1.52 | 39.31 | 3.47 | 37.87 | 6.88 | 49.12 | 7.91 | 96.91 | 9.76 | 93.62 | 13.63 |
| AD-1711756 | 20.57 | 4.05 | 23.96 | 2.75 | 36.33 | 11.33 | 72.94 | 13.08 | 54.99 | 8.99 | 85.20 | 23.87 | 99.26 | 9.01 |
| AD-1707605 | 35.03 | 2.49 | 46.37 | 9.61 | 55.57 | 13.31 | 83.69 | 7.72 | 96.68 | 15.17 | 95.74 | 27.12 | 110.00 | 19.41 |
| AD-1707287 | 27.61 | 1.70 | 32.48 | 1.14 | 47.24 | 9.39 | 73.48 | 6.92 | 70.18 | 9.33 | 93.02 | 11.38 | 110.85 | 27.51 |
| AD-1708766 | 19.22 | 3.37 | 24.71 | 4.87 | 34.20 | 7.62 | 60.19 | 7.39 | 69.81 | 13.98 | 92.36 | 11.05 | 125.22 | 12.99 |
| AD-1706644 | 18.94 | 2.78 | 25.14 | 3.46 | 49.00 | 1.82 | 68.06 | 11.84 | 83.45 | 4.42 | 87.08 | 12.23 | 127.39 | 7.76 |
| AD-1706590 | 24.14 | 4.90 | 34.95 | 3.80 | 53.60 | 6.42 | 78.09 | 12.28 | 113.12 | 26.95 | 114.41 | 16.33 | 135.34 | 15.94 |
| AD-1708407 | 24.10 | 7.20 | 30.72 | 4.39 | 43.60 | 6.30 | 74.49 | 11.38 | 70.25 | 8.69 | 90.22 | 13.32 | 105.77 | 8.20 |
| AD-1707292 | 24.48 | 4.27 | 34.78 | 2.52 | 45.46 | 9.22 | 72.04 | 12.03 | 58.89 | 13.58 | 86.65 | 5.87 | 86.26 | 19.87 |
| AD-1708899 | 17.77 | 2.91 | 21.87 | 1.95 | 26.43 | 7.65 | 68.65 | 14.69 | 99.52 | 14.24 | 94.06 | 11.30 | 111.70 | 15.07 |
| AD-1706674 | 20.19 | 2.23 | 26.54 | 1.38 | 47.96 | 2.84 | 70.03 | 6.72 | 69.05 | 8.66 | 102.69 | 11.08 | 104.89 | 21.93 |
| AD-1707631 | 20.66 | 2.86 | 19.79 | 4.30 | 32.43 | 5.70 | 61.81 | 11.29 | 71.35 | 13.55 | 93.00 | 11.79 | 126.82 | 9.10 |
| AD-1706759 | 18.55 | 2.17 | 26.28 | 2.28 | 40.19 | 5.23 | 63.77 | 4.79 | 77.91 | 12.04 | 97.13 | 8.57 | 111.16 | 27.11 |
| AD-1711747 | 11.52 | 4.71 | 17.00 | 2.82 | 33.59 | 6.89 | 55.08 | 11.20 | 85.92 | 27.07 | 98.80 | 9.94 | 133.36 | 9.56 |
| AD-1707149 | 15.95 | 3.01 | 28.26 | 5.99 | 40.68 | 5.31 | 57.51 | 12.07 | 83.78 | 12.31 | 102.28 | 8.63 | 121.17 | 7.59 |
| AD-1708251 | 31.99 | 5.46 | 30.05 | 3.30 | 44.02 | 7.05 | 65.77 | 7.52 | 59.12 | 18.47 | 100.89 | 12.16 | 103.22 | 24.69 |
| AD-1711776 | 38.30 | 15.35 | 48.19 | 5.85 | 63.98 | 12.66 | 90.37 | 9.23 | 85.76 | 14.78 | 97.44 | 13.61 | 110.66 | 12.48 |
| AD-1707315 | 22.73 | 2.34 | 24.01 | 5.64 | 46.41 | 5.45 | 84.80 | 11.81 | 72.86 | 8.03 | 102.13 | 20.54 | 110.56 | 7.38 |
| AD-1706750 | 18.16 | 1.56 | 20.63 | 4.42 | 29.43 | 10.64 | 40.85 | 4.80 | 55.55 | 5.48 | 105.85 | 15.64 | 117.08 | 19.52 |
| AD-1706799 | 31.99 | 6.20 | 33.80 | 5.58 | 46.45 | 8.79 | 90.17 | 12.69 | 101.66 | 26.60 | 111.12 | 20.30 | 133.05 | 18.74 |
| AD-1708485 | 13.61 | 3.64 | 23.72 | 2.33 | 52.65 | 11.16 | 73.70 | 2.25 | 77.15 | 6.87 | 103.41 | 11.13 | 127.34 | 12.36 |
| AD-1707633 | 23.63 | 5.15 | 28.24 | 4.65 | 36.48 | 6.12 | 75.57 | 11.01 | 110.02 | 45.47 | 112.62 | 17.17 | 128.91 | 16.41 |
| AD-1709090 | 20.30 | 2.87 | 24.48 | 3.75 | 37.02 | 8.76 | 77.61 | 7.95 | 99.11 | 14.22 | 114.49 | 22.90 | 117.89 | 14.16 |
| AD-1707466 | 63.36 | 14.56 | 61.06 | 10.24 | 79.45 | 2.15 | 92.63 | 22.18 | 85.04 | 23.68 | 98.11 | 26.25 | 92.94 | 9.69 |
| AD-1711767 | 24.16 | 3.71 | 28.27 | 4.36 | 42.24 | 10.42 | 60.63 | 4.45 | 72.25 | 7.49 | 96.33 | 15.95 | 122.97 | 18.33 |
| AD-1707388 | 61.29 | 14.42 | 59.32 | 8.57 | 69.95 | 15.09 | 95.33 | 7.44 | 105.65 | 6.00 | 113.84 | 11.51 | 128.35 | 23.12 |
| AD-1711770 | 18.87 | 3.61 | 20.46 | 5.47 | 32.18 | 8.80 | 57.75 | 16.97 | 61.76 | 7.82 | 104.71 | 7.77 | 122.13 | 13.61 |
| AD-1706748 | 24.20 | 6.04 | 35.62 | 6.64 | 50.19 | 11.08 | 78.53 | 12.43 | 88.65 | 8.77 | 105.57 | 16.18 | 128.56 | 25.42 |
| AD-1707307 | 26.97 | 4.34 | 35.22 | 4.99 | 63.75 | 9.28 | 59.79 | 4.39 | 75.85 | 8.88 | 101.29 | 7.21 | 120.76 | 17.30 |
| AD-1707302 | 20.32 | 7.78 | 29.18 | 5.74 | 45.72 | 7.23 | 63.84 | 7.84 | 76.67 | 4.98 | 112.01 | 4.31 | 136.13 | 16.48 |
| AD-1709126 | 27.45 | 10.18 | 32.34 | 4.21 | 57.05 | 10.57 | 53.83 | 6.16 | 49.10 | 5.95 | 88.09 | 16.28 | 112.27 | 11.26 |
| AD-1711765 | 17.66 | 4.49 | 22.90 | 2.22 | 28.06 | 8.93 | 40.32 | 5.52 | 60.56 | 12.85 | 94.87 | 19.12 | 99.62 | 14.72 |
| AD-1707140 | 14.92 | 5.79 | 15.70 | 5.48 | 29.72 | 5.60 | 45.36 | 2.81 | 60.43 | 8.25 | 51.92 | 22.84 | 106.75 | 10.76 |
| AD-1706751 | 19.74 | 4.12 | 19.51 | 8.94 | 51.31 | 13.73 | 31.89 | 5.76 | 61.33 | 16.59 | 86.50 | 16.71 | 119.06 | 17.21 |
| AD-1706930 | 18.10 | 6.15 | 17.27 | 4.50 | 29.73 | 6.81 | 44.91 | 14.50 | 53.16 | 2.39 | 92.88 | 11.31 | 94.54 | 12.43 |
| AD-1708564 | 33.34 | 10.11 | 34.73 | 8.99 | 65.01 | 8.10 | 68.81 | 10.42 | 82.59 | 24.87 | 107.17 | 6.26 | 105.04 | 13.30 |
| AD-1706746 | 28.39 | 9.86 | 38.43 | 9.94 | 65.72 | 12.30 | 56.28 | 12.49 | 81.09 | 8.55 | 106.87 | 14.96 | 91.27 | 18.95 |
| AD-1706863 | 21.83 | 8.44 | 29.55 | 2.92 | 39.21 | 8.12 | 64.73 | 12.20 | 61.97 | 17.33 | 61.21 | 5.92 | 91.41 | 19.42 |
| AD-1706933 | 19.52 | 6.57 | 24.12 | 4.64 | 36.58 | 13.09 | 52.18 | 9.14 | 60.25 | 8.05 | 76.05 | 5.15 | 89.07 | 34.13 |
| AD-1706650 | 8.22 | 2.78 | 9.86 | 1.53 | 18.29 | 4.10 | 18.41 | 2.70 | 31.54 | 9.06 | 47.21 | 2.59 | 82.80 | 6.30 |
| AD-1706645 | 9.54 | 2.94 | 13.04 | 1.86 | 19.68 | 5.15 | 23.19 | 1.25 | 31.74 | 9.21 | 58.68 | 3.98 | 75.47 | 14.03 |
| AD-1706763 | 8.78 | 2.73 | 10.89 | 2.20 | 18.03 | 2.25 | 24.39 | 5.06 | 32.66 | 2.25 | 57.89 | 9.13 | 83.09 | 3.30 |
| AD-1706642 | 10.21 | 2.41 | 14.73 | 4.43 | 29.08 | 10.15 | 26.47 | 6.44 | 44.93 | 12.38 | 65.76 | 1.34 | 80.97 | 14.28 |
| AD-1706749 | 10.78 | 3.21 | 18.58 | 2.61 | 29.08 | 1.90 | 40.07 | 4.58 | 45.28 | 6.05 | 78.38 | 3.11 | 70.35 | 25.84 |
| AD-1709044 | 12.38 | 3.44 | 19.35 | 3.08 | 27.49 | 7.29 | 39.88 | 7.52 | 48.75 | 6.09 | 70.84 | 5.85 | 82.43 | 6.36 |
| AD-1707063 | 6.22 | 2.57 | 4.60 | 1.96 | 11.68 | 3.24 | 12.46 | 0.47 | 15.19 | 1.73 | 42.37 | 9.63 | 53.30 | 26.27 |
| AD-1706666 | 11.63 | 2.89 | 10.71 | 2.18 | 16.80 | 4.52 | 21.12 | 6.46 | 28.56 | 5.28 | 45.18 | 3.28 | 27.94 | 3.80 |
| AD-1707305 | 10.26 | 4.05 | 7.94 | 3.22 | 10.70 | 3.10 | 15.00 | 2.72 | 19.14 | 2.01 | 52.56 | 17.22 | 58.51 | 0.00 |
| AD-1707060 | 8.16 | 3.68 | 9.02 | 2.54 | 18.61 | 4.71 | 29.10 | 5.61 | 37.29 | 2.54 | 51.66 | 11.49 | 43.46 | 16.10 |
| AD-1707318 | 8.67 | 3.65 | 8.96 | 1.42 | 16.04 | 5.89 | 25.62 | 2.76 | 29.27 | 3.58 | 47.38 | 12.44 | 57.54 | 2.19 |
| AD-1707290 | 18.38 | 5.04 | 17.99 | 3.06 | 32.26 | 7.70 | 46.85 | 2.48 | 56.51 | 9.06 | 67.51 | 11.70 | 82.20 | 11.04 |
| AD-1711760 | 10.18 | 3.91 | 15.74 | 2.67 | 25.12 | 9.22 | 40.81 | 3.41 | 42.21 | 6.96 | 65.57 | 7.30 | 50.87 | 7.17 |

TABLE 21C
Single Dose Screen for dsRNA agents targeting INHBE in PCH cells (% average mRNA remaining).

| Duplex | Primary Cyno Hepatocytes Transfection | | | | | | Primary Cyno Hepatocytes Free Uptake | | | | | | | |
|---|---|---|---|---|---|---|---|---|---|---|---|---|---|---|
| Name | 10 nM | StDev | 1 nM | StDev | 0.1 nM | StDev | 250 nM | StDev | 100 nM | StDev | 10 nM | StDev | 1 nM | StDev |
| AD-1708473 | 7.47 | 2.74 | 32.98 | 4.49 | 38.46 | 5.22 | 5.83 | 1.24 | 7.90 | 0.68 | 13.73 | 3.81 | 36.44 | 9.10 |
| AD-1707061 | 5.91 | 1.59 | 25.97 | 2.80 | 37.95 | 4.74 | 4.93 | 1.33 | 6.19 | 1.17 | 12.75 | 1.19 | 24.67 | 3.29 |
| AD-1708478 | 10.87 | 1.82 | 34.31 | 4.82 | 44.21 | 8.99 | 8.33 | 1.99 | 9.75 | 1.98 | 12.20 | 3.03 | 22.11 | 6.43 |
| AD-1706584 | 13.05 | 2.93 | 32.59 | 4.71 | 35.84 | 5.03 | 8.45 | 2.09 | 11.65 | 2.82 | 17.02 | 1.74 | 33.73 | 6.39 |
| AD-1707306 | 9.51 | 0.94 | 34.07 | 4.86 | 43.92 | 7.44 | 11.65 | 1.92 | 8.48 | 1.71 | 14.04 | 3.13 | 39.14 | 5.62 |
| AD-1711744 | 18.10 | 4.00 | 42.42 | 4.03 | 53.93 | 8.59 | 8.55 | 2.85 | 10.68 | 2.12 | 13.50 | 4.06 | 38.67 | 0.25 |
| AD-1707639 | 18.01 | 2.14 | 45.23 | 3.14 | 57.21 | 6.84 | 16.87 | 2.63 | 13.75 | 2.87 | 20.90 | 6.68 | 37.20 | 1.37 |
| AD-1707641 | 14.45 | 1.14 | 36.97 | 4.67 | 53.00 | 3.54 | 11.06 | 1.98 | 12.53 | 1.52 | 24.21 | 4.94 | 41.21 | 5.67 |
| AD-1707640 | 20.53 | 3.28 | 37.16 | 9.19 | 62.35 | 9.81 | 15.05 | 3.30 | 20.19 | 1.59 | 24.58 | 4.89 | 25.42 | 2.79 |
| AD-1706583 | 14.48 | 3.67 | 47.86 | 4.86 | 61.48 | 8.01 | 10.32 | 3.93 | 15.82 | 6.07 | 18.61 | 4.66 | 48.58 | 11.62 |
| AD-1708956 | 11.09 | 2.08 | 35.23 | 5.58 | 50.07 | 7.79 | 9.32 | 0.27 | 11.90 | 2.85 | 24.94 | 5.33 | 45.15 | 8.24 |
| AD-1706591 | 19.64 | 2.79 | 56.40 | 7.78 | 62.31 | 6.78 | 9.18 | 1.92 | 15.12 | 0.82 | 25.21 | 7.00 | 62.16 | 9.52 |
| AD-1706593 | 18.22 | 3.15 | 50.39 | 3.94 | 50.47 | 8.38 | 9.42 | 2.42 | 11.27 | 2.59 | 23.69 | 6.94 | 50.88 | 10.01 |
| AD-1707136 | 10.23 | 2.58 | 27.34 | 6.99 | 46.91 | 8.54 | 9.04 | 1.81 | 8.49 | 1.70 | 21.51 | 2.16 | 42.87 | 4.46 |
| AD-1706587 | 23.87 | 3.81 | 46.95 | 8.49 | 62.57 | 11.27 | 14.48 | 2.31 | 15.69 | 2.72 | 25.82 | 4.63 | 47.27 | 5.35 |
| AD-1706547 | 29.86 | 3.63 | 53.87 | 10.28 | 66.80 | 3.64 | 23.99 | 4.15 | 26.80 | 8.35 | 46.70 | 7.85 | 64.97 | 16.25 |
| AD-1708399 | 24.39 | 4.14 | 56.93 | 4.99 | 62.91 | 13.62 | 12.07 | 3.35 | 13.83 | 4.69 | 26.04 | 4.79 | 66.91 | 13.04 |
| AD-1707340 | 39.28 | 4.82 | 64.21 | 4.81 | 79.59 | 4.44 | 29.23 | 6.08 | 32.92 | 12.21 | 38.14 | 5.42 | 71.22 | 22.52 |
| AD-1706662 | 14.59 | 0.79 | 39.23 | 10.93 | 58.74 | 14.45 | 16.76 | 3.53 | 14.74 | 1.00 | 30.41 | 4.29 | 60.37 | 3.75 |
| AD-1709130 | 16.24 | 2.78 | 52.70 | 10.74 | 65.15 | 10.76 | 17.57 | 3.03 | 18.14 | 2.88 | 27.68 | 4.76 | 65.67 | 7.63 |
| AD-1708882 | 16.97 | 1.09 | 45.63 | 12.78 | 77.02 | 4.42 | 11.89 | 0.90 | 12.60 | 2.44 | 27.58 | 2.96 | 58.25 | 5.90 |
| AD-1707087 | 18.19 | 1.93 | 38.32 | 10.98 | 74.09 | 4.20 | 17.93 | 3.41 | 18.11 | 2.79 | 37.96 | 5.53 | 69.05 | 2.24 |
| AD-1707632 | 25.08 | 3.09 | 40.42 | 13.55 | 58.16 | 4.73 | 18.92 | 0.75 | 17.82 | 6.37 | 27.71 | 7.00 | 53.11 | 5.97 |
| AD-1709034 | 26.18 | 2.74 | 62.45 | 7.84 | 69.43 | 9.15 | 14.25 | 1.45 | 13.43 | 4.36 | 22.01 | 7.95 | 42.74 | 7.41 |
| AD-1706761 | 14.04 | 2.46 | 48.49 | 12.33 | 57.31 | 7.84 | 11.03 | 0.09 | 9.71 | 1.51 | 22.26 | 3.89 | 83.78 | 32.45 |
| AD-1711775 | 43.13 | 4.73 | 79.00 | 6.98 | 88.54 | 8.11 | 44.74 | 6.00 | 49.54 | 8.66 | 54.24 | 14.15 | 104.57 | 8.08 |
| AD-1706758 | 32.06 | 5.59 | 68.22 | 14.20 | 82.73 | 11.67 | 22.92 | 5.17 | 22.67 | 2.26 | 40.00 | 0.42 | 73.16 | 9.75 |
| AD-1707339 | 39.24 | 2.12 | 74.96 | 0.78 | 68.46 | 12.48 | 33.30 | 3.28 | 34.74 | 9.08 | 54.47 | 10.26 | 74.36 | 15.15 |
| AD-1708368 | 40.73 | 3.67 | 67.92 | 16.66 | 91.79 | 9.70 | 23.02 | 3.99 | 26.31 | 3.29 | 39.13 | 4.09 | 76.42 | 15.36 |
| AD-1706762 | 28.37 | 6.96 | 62.89 | 6.26 | 77.63 | 9.41 | 14.52 | 1.86 | 14.28 | 1.73 | 35.29 | 4.03 | 60.12 | 9.31 |
| AD-1707291 | 23.69 | 2.29 | 43.10 | 5.34 | 63.68 | 14.11 | 20.65 | 2.71 | 15.40 | 3.39 | 31.37 | 3.02 | 62.75 | 15.07 |
| AD-1707314 | 29.79 | 6.31 | 65.15 | 5.49 | 83.38 | 12.47 | 21.26 | 5.85 | 25.31 | 2.95 | 28.72 | 3.30 | 80.36 | 4.21 |
| AD-1706640 | 35.22 | 5.23 | 62.17 | 10.30 | 72.73 | 4.13 | 12.56 | 2.49 | 13.90 | 2.35 | 31.22 | 3.54 | 67.25 | 0.70 |
| AD-1707054 | 19.36 | 3.19 | 54.28 | 2.70 | 68.42 | 3.25 | 15.34 | 2.10 | 19.26 | 3.32 | 36.61 | 5.50 | 73.41 | 6.83 |
| AD-1706641 | 38.81 | 2.77 | 83.23 | 5.90 | 81.54 | 10.41 | 26.69 | 3.72 | 24.16 | 7.74 | 49.31 | 6.53 | 98.76 | 9.65 |
| AD-1707308 | 31.18 | 3.14 | 67.03 | 11.46 | 72.91 | 13.64 | 17.47 | 5.59 | 25.13 | 0.65 | 29.27 | 3.23 | 81.25 | 8.44 |
| AD-1709151 | 58.99 | 7.07 | 99.08 | 24.13 | 82.51 | 23.90 | 49.15 | 8.11 | 35.96 | 12.08 | 52.50 | 11.94 | 106.94 | 11.16 |
| AD-1706656 | 22.28 | 2.36 | 52.59 | 5.56 | 69.36 | 9.76 | 11.40 | 2.25 | 12.08 | 3.81 | 29.92 | 2.75 | 46.55 | 4.66 |
| AD-1706651 | 31.17 | 5.34 | 55.09 | 7.98 | 50.19 | 9.10 | 12.74 | 1.10 | 12.86 | 2.14 | 28.96 | 2.78 | 58.95 | 12.54 |
| AD-1711756 | 22.78 | 4.88 | 51.83 | 14.00 | 73.32 | 15.55 | 18.21 | 2.09 | 20.58 | 2.82 | 35.07 | 2.15 | 65.70 | 19.08 |
| AD-1707605 | 51.27 | 4.38 | 77.10 | 19.46 | 83.78 | 3.65 | 48.36 | 10.82 | 47.85 | 10.00 | 49.56 | 7.16 | 93.66 | 26.70 |
| AD-1707287 | 17.93 | 2.05 | 48.59 | 9.40 | 63.94 | 7.11 | 19.30 | 2.43 | 21.57 | 2.40 | 32.78 | 6.36 | 57.70 | 4.49 |
| AD-1708766 | 18.81 | 3.17 | 55.43 | 5.57 | 70.17 | 4.47 | 15.34 | 2.89 | 17.95 | 2.34 | 25.44 | 3.89 | 64.23 | 13.01 |
| AD-1706644 | 28.89 | 6.40 | 51.39 | 16.56 | 79.04 | 6.62 | 26.48 | 2.52 | 32.64 | 2.30 | 49.95 | 1.49 | 81.75 | 8.97 |
| AD-1706590 | 38.15 | 1.61 | 80.31 | 6.44 | 92.30 | 6.41 | 26.35 | 3.32 | 25.15 | 5.92 | 40.61 | 6.35 | 78.62 | 8.57 |
| AD-1708407 | 35.96 | 4.47 | 67.88 | 12.42 | 94.43 | 8.89 | 17.03 | 2.89 | 19.27 | 4.04 | 42.22 | 5.00 | 64.72 | 15.64 |
| AD-1707292 | 39.27 | 5.95 | 72.26 | 26.50 | 73.87 | 16.12 | 21.97 | 3.63 | 28.47 | 3.81 | 42.02 | 6.40 | 83.02 | 16.74 |
| AD-1708899 | 37.10 | 1.50 | 74.69 | 9.73 | 88.87 | 10.06 | 19.00 | 6.82 | 25.47 | 3.31 | 50.32 | 9.44 | 81.40 | 2.23 |
| AD-1706674 | 32.84 | 9.12 | 64.73 | 4.42 | 83.67 | 6.57 | 23.52 | 5.70 | 28.16 | 5.66 | 35.56 | 3.67 | 79.83 | 4.59 |
| AD-1707631 | 17.72 | 3.24 | 32.29 | 8.79 | 73.51 | 6.20 | 19.06 | 3.32 | 21.57 | 2.84 | 30.74 | 2.52 | 57.82 | 7.61 |
| AD-1706759 | 22.82 | 5.97 | 59.65 | 15.92 | 81.70 | 12.26 | 15.52 | 1.03 | 15.75 | 2.69 | 31.88 | 3.25 | 64.72 | 9.61 |
| AD-1711747 | 16.88 | 1.38 | 51.75 | 5.39 | 77.38 | 9.80 | 7.61 | 1.11 | 10.05 | 2.18 | 18.90 | 2.56 | 45.02 | 6.23 |
| AD-1707149 | 30.43 | 7.20 | 68.03 | 10.50 | 95.47 | 10.74 | 12.97 | 2.94 | 15.39 | 4.97 | 31.35 | 3.87 | 51.94 | 2.96 |
| AD-1708251 | 67.20 | 2.14 | 75.30 | 12.49 | 80.77 | 12.76 | 28.28 | 6.61 | 37.65 | 10.05 | 51.75 | 12.05 | 78.36 | 9.89 |
| AD-1711776 | 42.99 | 0.86 | 80.17 | 9.26 | 89.90 | 2.55 | 33.31 | 3.56 | 39.81 | 7.57 | 61.94 | 4.12 | 88.88 | 14.71 |
| AD-1707315 | 31.05 | 8.92 | 70.52 | 5.55 | 98.15 | 8.08 | 36.35 | 6.21 | 33.91 | 4.76 | 46.61 | 2.99 | 86.97 | 17.28 |
| AD-1706750 | 20.76 | 2.52 | 61.32 | 8.84 | 71.39 | 12.47 | 16.65 | 1.24 | 15.84 | 3.69 | 29.05 | 2.00 | 65.00 | 9.30 |
| AD-1706799 | 27.06 | 4.43 | 65.37 | 6.49 | 84.62 | 11.72 | 36.11 | 8.14 | 36.55 | 9.82 | 46.21 | 10.11 | 87.85 | 10.61 |
| AD-1708485 | 21.96 | 3.70 | 48.80 | 10.16 | 86.49 | 16.04 | 18.55 | 5.12 | 24.80 | 4.25 | 28.45 | 4.00 | 72.48 | 20.64 |
| AD-1707633 | 21.02 | 2.82 | 47.90 | 7.29 | 60.86 | 3.25 | 17.84 | 4.49 | 22.25 | 9.40 | 25.12 | 5.14 | 58.59 | 13.07 |
| AD-1709090 | 21.63 | 1.82 | 53.58 | 8.28 | 75.93 | 13.41 | 25.00 | 6.30 | 24.74 | 12.69 | 36.00 | 2.38 | 65.83 | 23.02 |
| AD-1707466 | 72.22 | 20.46 | 96.23 | 13.67 | 89.94 | 17.07 | 62.02 | 9.68 | 63.83 | 11.34 | 82.95 | 20.07 | 89.09 | 5.98 |
| AD-1711767 | 30.18 | 5.67 | 66.05 | 9.01 | 71.99 | 9.45 | 12.82 | 1.91 | 18.77 | 4.64 | 31.68 | 1.25 | 63.00 | 4.37 |
| AD-1707388 | 78.28 | 3.85 | 81.05 | 21.06 | 92.40 | 9.78 | 56.73 | 14.94 | 59.70 | 18.76 | 70.84 | 9.94 | 117.00 | 32.65 |
| AD-1711770 | 24.22 | 4.30 | 50.53 | 10.17 | 60.23 | 15.99 | 13.64 | 4.56 | 18.17 | 4.10 | 29.29 | 5.60 | 63.32 | 20.42 |
| AD-1706748 | 27.88 | 7.45 | 58.08 | 8.17 | 85.22 | 9.26 | 19.35 | 7.28 | 23.70 | 10.45 | 35.92 | 2.11 | 82.43 | 29.42 |
| AD-1707307 | 38.24 | 4.51 | 69.58 | 18.90 | 81.80 | 12.11 | 22.77 | 1.58 | 24.06 | 5.50 | 34.26 | 5.34 | 72.51 | 11.65 |
| AD-1707302 | 22.90 | 3.30 | 56.34 | 5.01 | 77.35 | 8.51 | 14.86 | 4.07 | 16.11 | 7.60 | 37.43 | 6.16 | 65.34 | 12.89 |
| AD-1709126 | 37.24 | 1.81 | 67.53 | 14.34 | 74.35 | 12.21 | 25.21 | 2.16 | 25.01 | 0.88 | 40.35 | 2.47 | 78.75 | 5.06 |
| AD-1711765 | 26.06 | 4.01 | 54.94 | 4.24 | 85.81 | 12.69 | 12.51 | 3.92 | 12.54 | 2.33 | 27.24 | 4.60 | 58.54 | 9.15 |
| AD-1707140 | 20.69 | 2.95 | 41.27 | 7.55 | 65.60 | 2.37 | 10.68 | 1.92 | 13.99 | 2.40 | 28.57 | 4.11 | 48.20 | 7.92 |
| AD-1706751 | 19.01 | 0.80 | 51.90 | 10.38 | 93.47 | 7.11 | 17.08 | 4.62 | 16.12 | 5.22 | 25.48 | 3.88 | 68.13 | 17.00 |
| AD-1706930 | 13.61 | 2.50 | 33.64 | 4.46 | 54.73 | 9.10 | 15.41 | 3.46 | 19.00 | 6.91 | 30.56 | 6.18 | 74.72 | 7.50 |
| AD-1708564 | 33.12 | 3.93 | 64.85 | 7.58 | 77.16 | 9.05 | 23.64 | 4.07 | 36.07 | 9.13 | 38.87 | 5.58 | 79.09 | 9.95 |
| AD-1706746 | 36.26 | 6.43 | 72.34 | 12.42 | 100.13 | 3.81 | 23.12 | 8.26 | 28.72 | 10.09 | 45.63 | 4.80 | 72.75 | 8.12 |
| AD-1706863 | 26.84 | 5.99 | 64.11 | 16.03 | 75.02 | 16.52 | 28.71 | 5.45 | 30.51 | 4.57 | 53.96 | 7.78 | 75.02 | 6.39 |
| AD-1706933 | 24.84 | 2.39 | 53.63 | 19.88 | 61.71 | 13.32 | 16.45 | 2.89 | 21.72 | 2.52 | 33.18 | 7.42 | 67.85 | 12.54 |
| AD-1706650 | 7.75 | 1.33 | 28.02 | 3.29 | 52.07 | 12.66 | 6.10 | 2.27 | 7.05 | 1.70 | 16.05 | 4.29 | 37.57 | 4.81 |
| AD-1706645 | 10.74 | 0.62 | 29.43 | 8.71 | 52.47 | 2.06 | 7.63 | 1.56 | 9.23 | 1.42 | 17.05 | 1.33 | 42.18 | 7.55 |
| AD-1706763 | 7.28 | 0.69 | 31.38 | 8.79 | 53.36 | 11.31 | 6.52 | 0.85 | 8.04 | 2.06 | 14.76 | 2.55 | 36.65 | 8.26 |
| AD-1706642 | 8.66 | 3.81 | 30.56 | 5.40 | 54.72 | 4.19 | 8.78 | 3.63 | 11.66 | 6.36 | 16.43 | 3.88 | 36.47 | 4.72 |
| AD-1706749 | 10.66 | 1.62 | 37.02 | 7.09 | 59.79 | 5.94 | 10.16 | 0.80 | 9.50 | 2.34 | 20.32 | 2.72 | 48.14 | 5.76 |
| AD-1709044 | 17.47 | 3.75 | 34.89 | 11.05 | 65.44 | 14.86 | 17.68 | 6.18 | 13.10 | 2.45 | 29.34 | 3.15 | 50.29 | 6.25 |
| AD-1707063 | 8.70 | 1.45 | 29.38 | 5.78 | 47.59 | 2.97 | 7.93 | 1.13 | 8.36 | 1.64 | 21.41 | 1.43 | 32.97 | 9.60 |
| AD-1706666 | 16.48 | 2.20 | 40.53 | 4.15 | 60.54 | 0.68 | 15.42 | 6.42 | 15.54 | 5.16 | 40.29 | 3.51 | 35.82 | 2.33 |
| AD-1707305 | 9.42 | 1.49 | 32.49 | 3.03 | 58.24 | 8.28 | 6.79 | 2.33 | 8.64 | 2.70 | 18.06 | 4.38 | 29.85 | 7.65 |
| AD-1707060 | 17.25 | 1.90 | 46.73 | 5.87 | 61.56 | 4.71 | 9.86 | 4.04 | 10.63 | 3.23 | 25.87 | 2.46 | 44.20 | 13.53 |
| AD-1707318 | 13.61 | 2.20 | 42.86 | 9.79 | 54.22 | 9.49 | 10.33 | 3.55 | 11.95 | 3.18 | 23.43 | 2.08 | 36.27 | 12.76 |
| AD-1707290 | 17.86 | 5.21 | 49.67 | 8.59 | 62.49 | 5.49 | 14.25 | 3.65 | 13.86 | 4.57 | 36.67 | 7.08 | 43.04 | 4.48 |
| AD-1711760 | 18.10 | 3.22 | 45.83 | 12.27 | 61.36 | 9.28 | 10.80 | 1.65 | 11.13 | 3.37 | 34.34 | 3.50 | 43.15 | 11.26 |

### Example 5: In Vitro Single Dose Screening of dsRNA Duplexes Targeting PDE3B

Using methods as described above, the dsRNA agents targeting PDE3B listed in Tables 14 and 15 were screened in vitro in Hep3B cells. The results of the single dose screen are shown in Table 22.

TABLE 22
Single dose screen for dsRNA agents
targeting PDE3B in Hep3b cells

PDE3B/GAPDH
10 nM

| Duplex Name | % Avg message remaining (n = 4) | SD |
|---|---|---|
| AD-1735860.1 | 23.709 | 2.696 |
| AD-1735859.1 | 20.551 | 5.347 |
| AD-1735858.1 | 14.679 | 1.474 |
| AD-1735857.1 | 16.078 | 1.257 |
| AD-1735856.1 | 64.047 | 7.135 |
| AD-1735855.1 | 18.703 | 1.706 |
| AD-1735854.1 | 91.214 | 2.665 |
| AD-1735853.1 | 61.829 | 7.675 |
| AD-1735852.1 | 24.913 | 1.561 |
| AD-1735851.1 | 24.650 | 1.255 |
| AD-1735850.1 | 51.046 | 2.446 |
| AD-1735849.1 | 40.548 | 3.364 |
| AD-1735848.1 | 71.080 | 3.478 |
| AD-1735847.1 | 28.692 | 1.123 |
| AD-1735846.1 | 19.800 | 1.135 |
| AD-1735845.1 | 22.788 | 1.056 |
| AD-1735844.1 | 44.102 | 0.526 |
| AD-1735843.1 | 52.791 | 8.362 |
| AD-1735842.1 | 35.622 | 1.690 |
| AD-1735841.1 | 18.613 | 2.422 |
| AD-1735840.1 | 57.636 | 0.785 |
| AD-1735839.1 | 97.956 | 4.340 |
| AD-1735838.1 | 40.270 | 2.411 |
| AD-1735837.1 | 30.805 | 1.988 |
| AD-1735836.1 | 35.379 | 6.026 |
| AD-1735835.1 | 26.142 | 3.567 |
| AD-1735834.1 | 35.490 | 3.096 |
| AD-1735833.1 | 64.925 | 1.880 |
| AD-1735832.1 | 21.201 | 2.311 |
| AD-1735831.1 | 37.417 | 4.395 |
| AD-1735830.1 | 23.780 | 2.967 |
| AD-1735829.1 | 40.465 | 2.494 |
| AD-1735828.1 | 53.338 | 1.805 |
| AD-1735827.1 | 23.074 | 6.126 |
| AD-1735826.1 | 61.743 | 0.745 |
| AD-1735825.1 | 42.766 | 14.280 |
| AD-1735824.1 | 19.157 | 0.711 |
| AD-1735823.1 | 38.720 | 1.905 |
| AD-1735822.1 | 71.730 | 3.742 |
| AD-1735821.1 | 37.215 | 0.923 |
| AD-1735820.1 | 24.961 | 0.995 |
| AD-1735819.1 | 16.917 | 0.728 |
| AD-1735818.1 | 46.222 | 0.902 |
| AD-1735817.1 | 61.037 | 0.700 |
| AD-1735816.1 | 35.774 | 2.507 |
| AD-1735815.1 | 43.474 | 2.475 |
| AD-1735814.1 | 45.076 | 0.985 |
| AD-1735813.1 | 28.507 | 1.743 |
| AD-1735812.1 | 70.709 | 1.627 |
| AD-1735811.1 | 22.367 | 0.715 |
| AD-1735810.1 | 32.837 | 1.316 |
| AD-1735809.1 | 19.024 | 1.358 |
| AD-1735808.1 | 37.406 | 3.104 |
| AD-1735807.1 | 39.498 | 3.599 |
| AD-1735806.1 | 32.960 | 1.566 |
| AD-1735805.1 | 76.711 | 1.049 |
| AD-1735804.1 | 30.205 | 1.234 |
| AD-1735803.1 | 29.615 | 0.884 |
| AD-1735802.1 | 89.553 | 2.905 |
| AD-1735801.1 | 35.954 | 0.721 |
| AD-1735800.1 | 22.730 | 2.119 |
| AD-1735799.1 | 38.084 | 2.611 |
| AD-1735798.1 | 21.808 | 1.760 |
| AD-1735797.1 | 39.127 | 5.754 |
| AD-1735796.1 | 24.799 | 3.324 |
| AD-1735795.1 | 29.095 | 2.968 |
| AD-1735794.1 | 74.630 | 2.670 |
| AD-1735793.1 | 50.655 | 2.625 |
| AD-1735792.1 | 21.774 | 1.922 |
| AD-1735791.1 | 29.879 | 1.379 |
| AD-1735790.1 | 45.278 | 3.178 |
| AD-1735789.1 | 53.799 | 3.522 |
| AD-1735788.1 | 24.525 | 2.272 |
| AD-1735787.1 | 41.825 | 5.048 |
| AD-1735786.1 | 47.854 | 2.516 |
| AD-1735785.1 | 24.928 | 5.836 |
| AD-1735784.1 | 26.802 | 2.165 |
| AD-1735783.1 | 22.593 | 2.437 |
| AD-1735782.1 | 51.096 | 3.906 |
| AD-1735781.1 | 25.026 | 1.459 |
| AD-1735780.1 | 17.158 | 1.252 |
| AD-1735779.1 | 93.272 | 6.172 |
| AD-1735778.1 | 24.207 | 1.738 |
| AD-1735777.1 | 21.304 | 0.659 |
| AD-1735776.1 | 23.915 | 5.465 |
| AD-1735775.1 | 21.095 | 1.525 |
| AD-1735774.1 | 51.845 | 2.742 |
| AD-1735773.1 | 28.834 | 5.357 |
| AD-1735772.1 | 22.993 | 2.710 |
| AD-1735771.1 | 21.214 | 1.330 |
| AD-1735770.1 | 29.426 | 7.369 |
| AD-1735769.1 | 50.021 | 9.727 |
| AD-1735768.1 | 51.776 | 2.906 |
| AD-1735767.1 | 66.112 | 13.788 |
| AD-1735766.1 | 48.132 | 2.745 |
| AD-1735765.1 | 78.926 | 8.535 |
| AD-1735764.1 | 31.425 | 6.159 |
| AD-1735763.1 | 39.086 | 4.992 |
| AD-1735762.1 | 28.522 | 1.684 |
| AD-1735761.1 | 24.999 | 1.443 |
| AD-1735760.1 | 100.922 | 13.187 |
| AD-1735759.1 | 44.830 | 2.225 |
| AD-1735758.1 | 24.656 | 3.386 |
| AD-1735757.1 | 39.249 | 2.608 |
| AD-1735756.1 | 25.038 | 2.266 |
| AD-1735755.1 | 59.725 | 1.902 |
| AD-1735754.1 | 58.172 | 3.336 |
| AD-1735753.1 | 19.504 | 1.090 |
| AD-1735752.1 | 39.414 | 3.022 |
| AD-1735751.1 | 99.329 | 6.328 |
| AD-1735750.1 | 20.627 | 0.706 |
| AD-1735749.1 | 46.816 | 1.166 |
| AD-1735748.1 | 28.579 | 1.052 |
| AD-1735747.1 | 79.882 | 6.062 |
| AD-1735746.1 | 22.412 | 1.094 |
| AD-1735745.1 | 35.269 | 1.846 |
| AD-1735744.1 | 80.796 | 4.208 |
| AD-1735743.1 | 16.948 | 0.992 |
| AD-1735742.1 | 33.029 | 1.186 |
| AD-1735741.1 | 20.754 | 1.851 |
| AD-1735740.1 | 65.848 | 4.934 |
| AD-1735739.1 | 29.858 | 0.742 |
| AD-1735738.1 | 15.106 | 1.527 |
| AD-1735737.1 | 18.822 | 0.946 |
| AD-1735736.1 | 21.250 | 2.499 |
| AD-1735735.1 | 29.588 | 2.812 |
| AD-1735734.1 | 40.456 | 4.030 |
| AD-1735733.1 | 32.308 | 6.658 |
| AD-1735732.1 | 84.880 | 4.774 |
| AD-1735731.1 | 92.758 | 5.134 |
| AD-1735730.1 | 30.568 | 2.601 |
| AD-1735729.1 | 25.859 | 6.962 |
| AD-1735728.1 | 14.793 | 0.736 |
| AD-1735727.1 | 24.247 | 2.191 |
| AD-1735726.1 | 19.711 | 2.888 |
| AD-1735725.1 | 26.066 | 0.946 |
| AD-1735724.1 | 21.329 | 0.845 |
| AD-1735723.1 | 41.212 | 5.232 |
| AD-1735722.1 | 19.123 | 1.479 |
| AD-1735721.1 | 32.446 | 2.942 |
| AD-1735720.1 | 27.006 | 6.384 |
| AD-1735719.1 | 24.997 | 8.940 |
| AD-1735718.1 | 19.553 | 2.845 |
| AD-1735717.1 | 47.688 | 16.341 |
| AD-1735716.1 | 20.980 | 2.028 |
| AD-1735715.1 | 41.194 | 6.384 |
| AD-1735714.1 | 25.154 | 4.575 |
| AD-1735713.1 | 17.184 | 2.579 |
| AD-1735712.1 | 17.501 | 1.079 |
| AD-1735711.1 | 30.023 | 3.460 |
| AD-1735710.1 | 25.387 | 5.162 |
| AD-1735709.1 | 62.222 | 7.071 |
| AD-1735708.1 | 85.988 | 22.851 |
| AD-1735707.1 | 20.219 | 2.756 |
| AD-1735706.1 | 18.664 | 3.260 |
| AD-1735705.1 | 25.208 | 2.956 |
| AD-1735704.1 | 20.478 | 1.354 |
| AD-1735703.1 | 22.386 | 4.605 |
| AD-1735702.1 | 65.465 | 18.354 |
| AD-1735701.1 | 58.432 | 21.264 |
| AD-1735700.1 | 29.458 | 1.405 |
| AD-1735699.1 | 26.141 | 2.115 |
| AD-1735698.1 | 25.475 | 1.590 |
| AD-1735697.1 | 110.116 | 4.516 |
| AD-1735696.1 | 21.957 | 2.061 |
| AD-1735695.1 | 38.524 | 7.895 |
| AD-1735694.1 | 16.352 | 2.076 |
| AD-1735693.1 | 13.906 | 2.757 |
| AD-1735692.1 | 87.266 | 12.920 |
| AD-1735691.1 | 57.983 | 4.032 |
| AD-1735690.1 | 62.142 | 3.477 |
| AD-1735689.1 | 84.924 | 5.580 |
| AD-1735688.1 | 17.170 | 1.339 |
| AD-1735687.1 | 19.041 | 1.800 |
| AD-1735686.1 | 37.229 | 1.697 |
| AD-1735685.1 | 63.412 | 8.597 |
| AD-1735684.1 | 82.100 | 4.966 |
| AD-1735683.1 | 19.608 | 4.567 |
| AD-1735682.1 | 39.393 | 5.573 |
| AD-1735681.1 | 28.252 | 3.787 |
| AD-1735680.1 | 20.977 | 1.302 |
| AD-1735679.1 | 26.540 | 1.005 |
| AD-1735678.1 | 75.941 | 10.521 |
| AD-1735677.1 | 71.242 | 12.310 |
| AD-1735676.1 | 14.192 | 4.284 |
| AD-1735675.1 | 25.581 | 2.905 |
| AD-1735674.1 | 18.237 | 1.270 |
| AD-1735673.1 | 49.950 | 4.698 |
| AD-1735672.1 | 45.190 | 4.895 |
| AD-1735671.1 | 48.600 | 14.146 |
| AD-1735670.1 | 27.972 | 1.043 |
| AD-1735669.1 | 37.706 | 4.548 |
| AD-1735668.1 | 21.295 | 2.205 |
| AD-1735667.1 | 20.943 | 1.604 |
| AD-1735666.1 | 22.679 | 0.591 |
| AD-1735665.1 | 23.466 | 3.781 |
| AD-1735664.1 | 21.604 | 7.639 |
| AD-1735663.1 | 18.338 | 4.288 |
| AD-1735662.1 | 58.417 | 7.253 |
| AD-1735661.1 | 21.512 | 1.617 |
| AD-1735660.1 | 17.670 | 1.798 |
| AD-1735659.1 | 28.972 | 5.188 |
| AD-1735658.1 | 27.689 | 0.477 |
| AD-1735657.1 | 75.794 | 6.235 |
| AD-1735656.1 | 21.802 | 5.378 |
| AD-1735655.1 | 19.501 | 1.729 |
| AD-1735654.1 | 23.484 | 2.101 |
| AD-1735653.1 | 63.740 | 33.435 |
| AD-1735652.1 | 27.984 | 3.477 |
| AD-1735651.1 | 39.385 | 1.397 |
| AD-1735650.1 | 15.347 | 0.813 |
| AD-1735649.1 | 25.165 | 2.325 |
| AD-1735648.1 | 33.073 | 2.315 |
| AD-1735647.1 | 16.820 | 1.985 |
| AD-1735646.1 | 55.994 | 5.828 |
| AD-1735645.1 | 12.154 | 1.921 |
| AD-1735644.1 | 17.745 | 1.643 |
| AD-1735643.1 | 13.923 | 3.189 |
| AD-1735642.1 | 55.255 | 2.792 |
| AD-1735641.1 | 48.249 | 6.537 |
| AD-1735640.1 | 18.494 | 0.962 |
| AD-1735639.1 | 27.638 | 2.892 |
| AD-1735638.1 | 47.689 | 6.093 |
| AD-1735637.1 | 38.073 | 5.322 |
| AD-1735636.1 | 19.722 | 3.753 |
| AD-1735635.1 | 32.670 | 1.578 |
| AD-1735634.1 | 18.063 | 1.428 |
| AD-1735633.1 | 16.891 | 2.258 |
| AD-1735632.1 | 95.524 | 0.685 |
| AD-1735631.1 | 40.879 | 4.633 |
| AD-1735630.1 | 26.068 | 3.356 |
| AD-1735629.1 | 19.330 | 2.600 |
| AD-1735628.1 | 17.470 | 1.514 |
| AD-1735627.1 | 45.620 | 5.372 |
| AD-1735626.1 | 30.086 | 2.595 |
| AD-1735625.1 | 47.252 | 6.513 |
| AD-1735624.1 | 83.854 | 10.000 |
| AD-1735623.1 | 22.800 | 1.603 |
| AD-1735622.1 | 16.870 | 1.728 |
| AD-1735621.1 | 32.332 | 9.786 |
| AD-1735620.1 | 42.507 | 1.603 |
| AD-1735619.1 | 89.434 | 2.676 |
| AD-1735618.1 | 25.773 | 2.560 |
| AD-1735617.1 | 35.284 | 4.815 |
| AD-1735616.1 | 13.217 | 1.679 |
| AD-1735615.1 | 35.212 | 5.461 |
| AD-1735614.1 | 13.061 | 1.196 |
| AD-1735613.1 | 21.191 | 1.016 |
| AD-1735612.1 | 62.200 | 9.169 |
| AD-1735611.1 | 17.737 | 2.166 |
| AD-1735610.1 | 46.749 | 2.389 |
| AD-1735609.1 | 20.890 | 1.371 |
| AD-1735608.1 | 16.779 | 2.166 |
| AD-1735607.1 | 10.409 | 0.676 |
| AD-1735606.1 | 34.953 | 10.155 |
| AD-1735605.1 | 81.087 | 8.632 |
| AD-1735604.1 | 53.898 | 11.426 |
| AD-1735603.1 | 34.331 | 3.868 |
| AD-1735602.1 | 35.125 | 3.978 |
| AD-1735601.1 | 17.106 | 0.910 |
| AD-1735600.1 | 38.242 | 3.303 |
| AD-1735599.1 | 10.192 | 2.638 |
| AD-1735598.1 | 67.145 | 9.671 |
| AD-1735597.1 | 25.931 | 3.096 |
| AD-1735596.1 | 70.287 | 11.476 |
| AD-1735595.1 | 70.859 | 20.399 |
| AD-1735594.1 | 14.430 | 0.732 |
| AD-1735593.1 | 20.530 | 2.301 |
| AD-1735592.1 | 45.267 | 10.242 |
| AD-1735591.1 | 14.976 | 0.539 |

### Example 6: In Vitro Single Dose Screening of dsRNA Duplexes Targeting INHBC

The dsRNA agents targeting INHBC listed in Tables 16 and 17 were screened in Hepa1-6 cells by an in vitro dual-Luciferase assay.

Briefly, Hepa1-6 cells were transfected by adding 50 μL of siRNA duplexes and 100 ng of a V180 plasmid, comprising human INHBC target sequence, nucleotides 1425-3202 of NM_005538.4, or 75 ng of a V179 plasmid, comprising human INHBC target sequence, nucleotides 1-1485 of NM_005538.4, per well along with 100 μL of Opti-MEM plus 0.5 μL of Lipofectamine 2000 per well (Invitrogen, Carlsbad CA. cat 13778-150) and then incubated at room temperature for 15 minutes. The mixture was then added to the cells which were re-suspended in 35 μL of fresh complete media. The transfected cells were incubated at 37° C. in an atmosphere of 5% CO2. Single-dose experiments were performed at 10 nM.

Twenty-four hours after the siRNAs and plasmid were transfected, Firefly (transfection control) and Renilla (fused to INHBC target sequence comprising nucleotides 1425-3202 or nucleotides 1-1485 of NM_005538.4) luciferase were measured. First, media was removed from the cells. Then, Firefly luciferase activity was measured by adding 75 μL of Dual-Glo® Luciferase Reagent equal to the culture medium volume to each well and mix. The mixture was incubated at room temperature for 30 minutes before luminescense (500 nm) was measured on a Spectramax (Molecular Devices) to detect the Firefly luciferase signal. Renilla luciferase activity was measured by adding 75 μL of room temperature of Dual-Glo® Stop & Glo® Reagent to each well and the plates were incubated for 10-15 minutes before luminescence was again measured to determine the Renilla luciferase signal. The Dual-Glo® Stop & Glo® Reagent quenches the firefly luciferase signal and sustained luminescence for the Renilla luciferase reaction. siRNA activity was determined by normalizing the Renilla (INHBC) signal to the Firefly (control) signal within each well. The magnitude of siRNA activity was then assessed relative to cells that were transfected with the same vector but were not treated with siRNA or were treated with a non-targeting siRNA. All transfections were done with n=4.

The results of the dual-luciferase assays of the agents are provided in Table 23.

TABLE 23
Dual-Luciferase Screen for dsRNA agents
targeting INHBC in Hepa1-6 Cells

| | | RLuc/FLuc | |
|---|---|---|---|
| Reporter | | 10 nM | |
| Plasmid | Duplex ID | % Avg message remaining (n = 4) | SD |
| V180 | AD-1732940.1 | 14.150 | 0.629 |
| | AD-1732908.1 | 7.967 | 0.476 |
| | AD-1732890.1 | 10.260 | 0.453 |
| | AD-1732885.1 | 8.392 | 0.401 |
| | AD-1732881.1 | 61.954 | 0.878 |
| | AD-1732865.1 | 16.807 | 0.670 |
| | AD-1732849.1 | 41.476 | 2.680 |
| | AD-1732827.1 | 14.425 | 0.743 |
| | AD-1732807.1 | 19.622 | 0.725 |
| | AD-1732786.1 | 17.152 | 0.302 |
| | AD-1732757.1 | 18.612 | 1.287 |
| | AD-1732744.1 | 16.097 | 1.579 |
| | AD-1732728.1 | 29.876 | 2.334 |
| | AD-1732708.1 | 68.958 | 7.072 |
| | AD-1732696.1 | 11.309 | 1.048 |
| | AD-1732677.1 | 10.846 | 0.810 |
| | AD-1732661.1 | 12.693 | 0.648 |
| | AD-1732644.1 | 21.739 | 2.433 |
| | AD-1732625.1 | 20.810 | 0.984 |
| | AD-1732604.1 | 34.719 | 1.881 |
| | AD-1732584.1 | 25.478 | 0.950 |
| | AD-1732565.1 | 23.217 | 1.123 |
| | AD-1732549.1 | 77.904 | 2.566 |
| | AD-1732533.1 | 55.404 | 2.637 |
| | AD-1732520.1 | 75.941 | 4.976 |
| | AD-1732500.1 | 60.074 | 4.736 |
| | AD-1732479.1 | 76.945 | 3.845 |
| | AD-1732469.1 | 28.398 | 4.223 |
| | AD-1732440.1 | 69.737 | 3.729 |
| | AD-1732415.1 | 79.748 | 4.738 |
| | AD-1732389.1 | 19.244 | 0.749 |
| | AD-1732372.1 | 12.871 | 1.092 |
| | AD-1732353.1 | 14.060 | 0.517 |
| | AD-1732335.1 | 16.929 | 0.643 |
| | AD-1732308.1 | 7.798 | 0.528 |
| | AD-1732291.1 | 16.073 | 0.324 |
| | AD-1732269.1 | 15.369 | 0.454 |
| | AD-1732253.1 | 11.966 | 0.396 |
| | AD-1732235.1 | 15.662 | 0.455 |
| | AD-1732217.1 | 11.891 | 0.307 |
| | AD-1732195.1 | 22.664 | 2.324 |
| | AD-1732176.1 | 40.545 | 3.354 |
| | AD-1732173.1 | 36.333 | 4.051 |
| | AD-1732157.1 | 12.425 | 0.688 |
| | AD-1732110.1 | 8.878 | 0.368 |
| | AD-1732094.1 | 16.277 | 0.992 |
| | AD-1732078.1 | 12.292 | 0.574 |
| | AD-1732062.1 | 13.286 | 1.417 |
| | AD-1732048.1 | 25.400 | 0.737 |
| | AD-1732031.1 | 12.699 | 1.522 |
| | AD-1732013.1 | 13.228 | 0.746 |
| | AD-1731997.1 | 14.695 | 2.144 |
| | AD-1731978.1 | 22.496 | 3.865 |
| | AD-1731958.1 | 20.145 | 0.926 |
| | AD-1731932.1 | 66.101 | 5.314 |
| | AD-1731923.1 | 74.139 | 8.370 |
| | AD-1731906.1 | 45.134 | 4.197 |
| | AD-1731890.1 | 33.397 | 2.478 |
| | AD-1731874.1 | 41.801 | 1.190 |
| | AD-1731858.1 | 4.851 | 0.510 |
| | AD-1731842.1 | 6.224 | 0.565 |
| | AD-1731819.1 | 11.143 | 0.815 |
| | AD-1731813.1 | 8.497 | 0.331 |
| | AD-1731782.1 | 4.415 | 0.145 |
| | AD-1731753.1 | 11.691 | 1.185 |
| | AD-1731750.1 | 5.301 | 0.362 |
| | AD-1731745.1 | 10.404 | 0.658 |
| | AD-1731728.1 | 22.012 | 2.859 |
| | AD-1731720.1 | 12.432 | 0.820 |
| | AD-1731704.1 | 5.547 | 0.321 |
| | AD-1731687.1 | 3.065 | 0.161 |
| | AD-1731671.1 | 19.915 | 2.526 |
| V179 | AD-1731704.1 | 15.257 | 0.906 |
| | AD-1731687.1 | 11.014 | 0.767 |
| | AD-1731671.1 | 34.407 | 2.796 |
| | AD-1731646.1 | 50.018 | 5.438 |
| | AD-1731618.1 | 15.753 | 1.031 |
| | AD-1731600.1 | 12.780 | 0.848 |
| | AD-1731584.1 | 8.732 | 0.637 |
| | AD-1731564.1 | 13.821 | 1.071 |
| | AD-1731562.1 | 9.409 | 0.428 |
| | AD-1731545.1 | 66.529 | 4.870 |
| | AD-1731527.1 | 92.440 | 1.602 |
| | AD-1731511.1 | 75.267 | 3.429 |
| | AD-1731492.1 | 9.940 | 0.588 |
| | AD-1731484.1 | 27.855 | 1.905 |
| | AD-1731457.1 | 18.170 | 2.945 |
| | AD-1731431.1 | 42.677 | 4.621 |
| | AD-1731415.1 | 12.748 | 1.092 |
| | AD-1731398.1 | 28.945 | 3.717 |
| | AD-1731380.1 | 48.750 | 4.591 |
| | AD-1731371.1 | 72.563 | 2.686 |
| | AD-1731358.1 | 65.200 | 3.035 |
| | AD-1731340.1 | 8.662 | 0.132 |
| | AD-1731323.1 | 43.722 | 2.197 |
| | AD-1731307.1 | 67.294 | 4.521 |
| | AD-1731289.1 | 52.881 | 4.906 |
| | AD-1731264.1 | 22.594 | 2.101 |
| | AD-1731234.1 | 55.843 | 3.247 |
| | AD-1731205.1 | 10.827 | 0.702 |
| | AD-1731189.1 | 18.905 | 0.211 |
| | AD-1731173.1 | 58.483 | 2.870 |
| | AD-1731156.1 | 82.335 | 3.005 |
| | AD-1731137.1 | 20.307 | 1.198 |
| | AD-1731134.1 | 80.176 | 2.738 |
| | AD-1731117.1 | 65.192 | 2.858 |
| | AD-1731088.1 | 68.296 | 3.034 |
| | AD-1731064.1 | 44.769 | 2.606 |
| | AD-1731042.1 | 50.015 | 2.644 |
| | AD-1731035.1 | 57.224 | 3.625 |
| | AD-1731019.1 | 98.342 | 5.416 |
| | AD-1731003.1 | 30.570 | 3.760 |
| | AD-1730982.1 | 20.454 | 3.248 |
| | AD-1730958.1 | 25.954 | 0.786 |
| | AD-1730942.1 | 19.353 | 1.173 |
| | AD-1730926.1 | 36.218 | 0.945 |
| | AD-1730910.1 | 9.242 | 0.575 |
| | AD-1730873.1 | 17.439 | 1.317 |
| | AD-1730857.1 | 7.533 | 0.374 |
| | AD-1730841.1 | 15.620 | 1.085 |
| | AD-1730825.1 | 35.996 | 1.675 |
| | AD-1730807.1 | 13.721 | 0.994 |
| | AD-1730790.1 | 8.641 | 0.196 |
| | AD-1730769.1 | 37.556 | 1.872 |
| | AD-1730765.1 | 32.573 | 2.368 |
| | AD-1730749.1 | 41.768 | 1.107 |
| | AD-1730726.1 | 37.291 | 1.641 |
| | AD-1730691.1 | 20.187 | 1.873 |
| | AD-1730667.1 | 36.466 | 4.387 |
| | AD-1730643.1 | 35.649 | 2.632 |
| | AD-1730636.1 | 90.291 | 3.709 |
| | AD-1730606.1 | 35.942 | 0.833 |
| | AD-1730588.1 | 34.769 | 1.603 |
| | AD-1730570.1 | 46.879 | 2.310 |
| | AD-1730554.1 | 10.475 | 0.681 |
| | AD-1730529.1 | 42.962 | 3.284 |
| | AD-1730512.1 | 25.624 | 1.247 |
| | AD-1730493.1 | 72.772 | 4.634 |
| | Positive control | 0.663 | 0.053 |

### Example 7: In Vitro Single Dose Screening of dsRNA Duplexes Targeting PLIN1

Using methods as described above, the dsRNA agents targeting PLIN1 listed in Tables 10 and 11 were screened in vitro in Hepa1-6 cells by the dual-Luciferase assay. The results of the single dose screen are shown in Table 24.

TABLE 24
Dual-Luciferase Screen for dsRNA agents
targeting PLIN1 in Hepa1-6 Cells

RLuc/FLuc
10 nM

| Duplex ID | % Avg message remaining | SD |
|---|---|---|
| AD-1735320.1 | 53.561 | 4.672 |
| AD-1735319.1 | 33.212 | 3.061 |
| AD-1735318.1 | 87.299 | 11.406 |
| AD-1735317.1 | 72.995 | 9.626 |
| AD-1735316.1 | 80.346 | 7.389 |
| AD-1735315.1 | 34.013 | 3.602 |
| AD-1735314.1 | 77.100 | 9.948 |
| AD-1735313.1 | 61.678 | 7.363 |
| AD-1735312.1 | 47.388 | 4.892 |
| AD-1735311.1 | 57.067 | 4.870 |
| AD-1735310.1 | 69.433 | 2.837 |
| AD-1735309.1 | 87.563 | 6.359 |
| AD-1735308.1 | 54.214 | 6.804 |
| AD-1735307.1 | 77.536 | 7.971 |
| AD-1735306.1 | 44.999 | 0.952 |
| AD-1735305.1 | 20.312 | 0.690 |
| AD-1735304.1 | 37.885 | 1.346 |
| AD-1735303.1 | 89.423 | 1.906 |
| AD-1735302.1 | 71.173 | 4.238 |
| AD-1735301.1 | 70.170 | 3.690 |
| AD-1735300.1 | 62.217 | 7.128 |
| AD-1735299.1 | 45.332 | 4.595 |
| AD-1735298.1 | 75.846 | 7.520 |
| AD-1735297.1 | 69.820 | 5.033 |
| AD-1735296.1 | 64.227 | 7.301 |
| AD-1735295.1 | 90.389 | 7.070 |
| AD-1735294.1 | 73.067 | 7.141 |
| AD-1735293.1 | 26.508 | 3.863 |
| AD-1735292.1 | 91.596 | 11.766 |
| AD-1735291.1 | 84.530 | 11.202 |
| AD-1735290.1 | 80.139 | 6.540 |
| AD-1735289.1 | 40.946 | 2.761 |
| AD-1735288.1 | 33.302 | 3.704 |
| AD-1735287.1 | 37.684 | 3.007 |
| AD-1735286.1 | 47.467 | 3.016 |
| AD-1735285.1 | 28.454 | 1.209 |
| AD-1735284.1 | 30.826 | 3.351 |
| AD-1735283.1 | 35.399 | 1.914 |
| AD-1735282.1 | 39.620 | 3.772 |
| AD-1735281.1 | 65.860 | 1.354 |
| AD-1735280.1 | 21.944 | 2.006 |
| AD-1735279.1 | 52.047 | 4.181 |
| AD-1735278.1 | 36.993 | 3.597 |
| AD-1735277.1 | 43.703 | 0.939 |
| AD-1735276.1 | 24.075 | 1.681 |
| AD-1735275.1 | 41.179 | 1.720 |
| AD-1735274.1 | 30.228 | 2.242 |
| AD-1735273.1 | 37.603 | 3.724 |
| AD-1735272.1 | 27.325 | 2.573 |
| AD-1735271.1 | 54.654 | 4.358 |
| AD-1735270.1 | 30.214 | 2.322 |
| AD-1735269.1 | 47.580 | 1.497 |
| AD-1735268.1 | 29.621 | 2.529 |
| AD-1735267.1 | 38.804 | 3.763 |
| AD-1735266.1 | 29.659 | 0.328 |
| AD-1735265.1 | 34.545 | 2.741 |
| AD-1735264.1 | 80.144 | 6.568 |
| AD-1735263.1 | 26.159 | 1.851 |
| AD-1735262.1 | 37.861 | 1.332 |
| AD-1735261.1 | 43.807 | 5.808 |
| AD-1735260.1 | 30.677 | 2.583 |
| AD-1735259.1 | 43.570 | 4.475 |
| AD-1735258.1 | 40.682 | 3.274 |
| AD-1735257.1 | 43.057 | 2.824 |
| AD-1735256.1 | 38.041 | 2.568 |
| AD-1735255.1 | 21.650 | 0.909 |
| AD-1735254.1 | 27.873 | 3.744 |
| AD-1735253.1 | 77.253 | 3.270 |
| AD-1735252.1 | 39.960 | 2.697 |
| AD-1735251.1 | 41.732 | 2.546 |
| AD-1735250.1 | 27.088 | 1.383 |
| AD-1735249.1 | 29.574 | 1.839 |
| AD-1735248.1 | 45.794 | 3.063 |
| AD-1735247.1 | 68.096 | 8.378 |
| AD-1735246.1 | 40.621 | 2.231 |
| AD-1735245.1 | 59.324 | 3.637 |
| AD-1735244.1 | 22.847 | 1.836 |
| AD-1735243.1 | 85.325 | 3.497 |
| AD-1735242.1 | 35.687 | 3.410 |
| AD-1735241.1 | 23.433 | 0.870 |
| AD-1735240.1 | 31.517 | 1.780 |
| AD-1735239.1 | 36.244 | 2.725 |
| AD-1735238.1 | 32.611 | 1.443 |
| AD-1735237.1 | 14.047 | 0.939 |
| AD-1735236.1 | 16.631 | 0.772 |
| AD-1735235.1 | 31.488 | 3.435 |
| AD-1735234.1 | 39.842 | 2.222 |
| AD-1735233.1 | 13.998 | 0.629 |
| AD-1735232.1 | 47.840 | 5.226 |
| AD-1735231.1 | 31.494 | 1.469 |
| AD-1735230.1 | 23.989 | 1.990 |
| AD-1735229.1 | 19.072 | 2.237 |
| AD-1735228.1 | 16.810 | 1.007 |
| AD-1735227.1 | 27.650 | 2.449 |
| AD-1735226.1 | 24.667 | 1.786 |
| AD-1735225.1 | 23.622 | 2.500 |
| AD-1735224.1 | 24.205 | 1.728 |
| AD-1735223.1 | 13.924 | 1.257 |
| AD-1735222.1 | 16.961 | 1.476 |
| AD-1735221.1 | 26.245 | 1.531 |
| AD-1735220.1 | 28.657 | 1.159 |
| AD-1735219.1 | 16.124 | 1.387 |
| AD-1735218.1 | 23.929 | 1.107 |
| AD-1735217.1 | 71.822 | 3.235 |
| AD-1735216.1 | 22.636 | 1.631 |
| AD-1735215.1 | 10.556 | 1.255 |
| AD-1735214.1 | 92.571 | 5.143 |
| AD-1735213.1 | 25.487 | 2.249 |
| AD-1735212.1 | 12.128 | 0.655 |
| AD-1735211.1 | 29.064 | 2.614 |
| AD-1735210.1 | 19.343 | 2.334 |
| AD-1735209.1 | 17.586 | 1.040 |
| AD-1735208.1 | 13.678 | 0.528 |
| AD-1735207.1 | 25.561 | 1.744 |
| AD-1735206.1 | 21.848 | 2.252 |
| AD-1735205.1 | 17.025 | 1.043 |
| AD-1735204.1 | 15.538 | 0.982 |
| AD-1735203.1 | 16.080 | 1.928 |
| AD-1735202.1 | 16.432 | 0.718 |
| AD-1735201.1 | 34.346 | 1.470 |
| AD-1735200.1 | 24.511 | 1.673 |
| AD-1735199.1 | 7.066 | 0.486 |
| AD-1735198.1 | 14.709 | 1.084 |
| AD-1735197.1 | 16.627 | 1.417 |
| AD-1735196.1 | 13.687 | 0.708 |
| AD-1735195.1 | 22.964 | 1.902 |
| AD-1735194.1 | 39.620 | 0.928 |
| AD-1735193.1 | 27.843 | 1.143 |
| AD-1735192.1 | 17.138 | 0.504 |
| AD-1735191.1 | 20.200 | 1.123 |
| AD-1735190.1 | 18.874 | 0.788 |
| AD-1735189.1 | 21.167 | 0.415 |
| AD-1735188.1 | 25.098 | 3.920 |
| AD-1735187.1 | 33.725 | 1.983 |
| AD-1735186.1 | 17.016 | 0.284 |
| positive control | 0.759 | 0.092 |

### Example 8. In Vivo Assessment of RNAi Agents in Non-Human Primates (NHP)

The pharmacodynamic activity of duplexes targeting INHBE was also assessed in vivo in non-human primates (NHP).

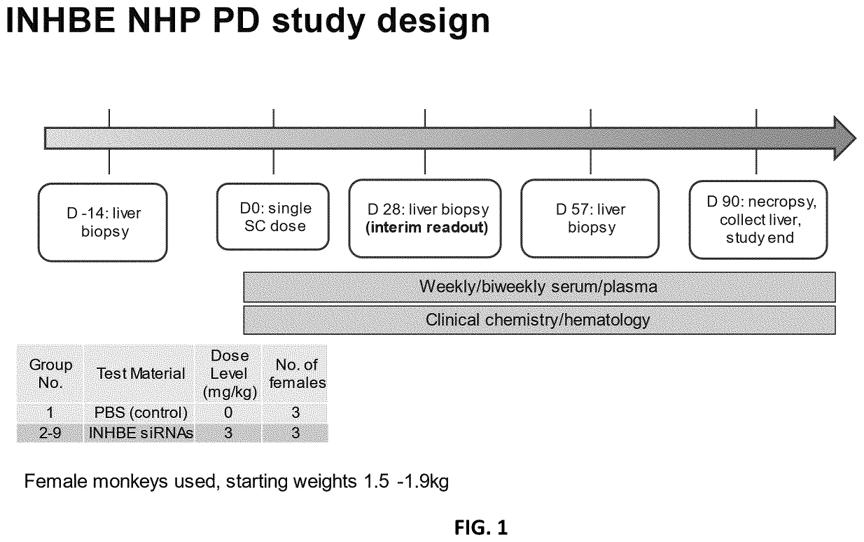

As depicted in FIG. 1, on Day 0 female non-human primates (n=3) were subcutaneously administered a single 3 mg/kg dose of AD-1707306, AD-1711744, AD-1708473, AD-1707640, AD-1707639, AD-1706583, AD-1706593, AD-1706761, or AD-1706662, or PBS control. Post-dose, plasma samples were collected biweekly, serum samples were collected weekly, and liver biopsy samples were collected at Days 28 and 57 post-dose to determine the effect of the agents on INHBE and INHBC mRNA expression, as described above, and clinical chemistry and hematological read-outs. Animals were sacrificed at Day 90 post-dose, tissue samples were collected and the levels of INHBE and INHBC mRNA were quantified as described above.

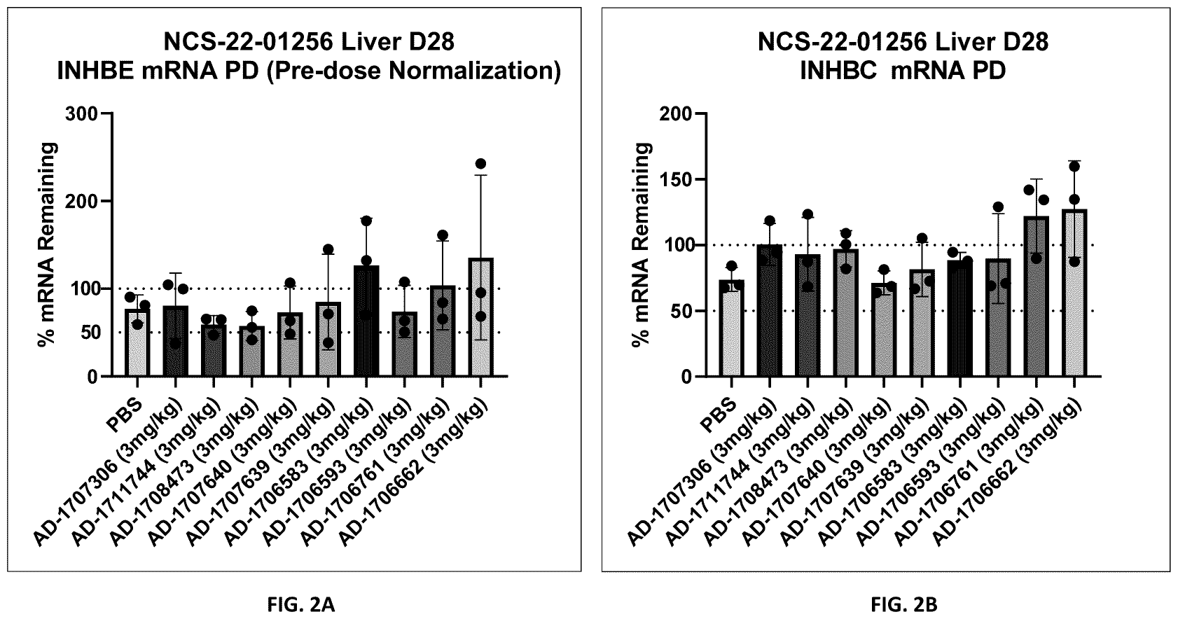

As depicted in FIGS. 2A and 2B, at Day 28 post-dose, a single 3 mg/kg subcutaneous dose of the agents potently inhibited INHBE expression in vivo (FIG. 2A) but not INHBC (FIG. 2B) expression.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1. A double stranded ribonucleic acid (dsRNA) agent for inhibiting expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC) in a cell,
wherein the dsRNA agent comprises a sense strand and an antisense strand forming a double stranded region,
a) wherein the metabolic disorder-associated target gene is INHBE and the antisense strand comprises at least 15 contiguous nucleotides differing by no more than 3 nucleotides from any one of the antisense strand nucleotide sequences selected from the group consisting of
SEQ ID NO: 6245
5′-AAAGACGGCAGAAUGGAAAGAGG-3′ of;
SEQ ID NO: 64
5′-AAGAAAGUAUAAAUGCUUGUCUC-3′ of;
SEQ ID NO: 63
5′-ACAGACAAGAAAGUGCCCAUUUG-3′ of;
SEQ ID NO: 58
5′-AGUUAUTCUGGGACGACUGGUCU-3′ of;
and
SEQ ID NO: 6403
5′-AAAGCCAGAGUCUCAGACAAGAA-3′ of.
and
wherein all of the nucleotides of the sense strand and all of the nucleotides of the antisense strand comprise a nucleotide modification
b) wherein the metabolic disorder-associated target gene is INHBE and the antisense strand comprises at least 15 contiguous nucleotides differing by no more than three nucleotides from the complement of any one of the nucleotide sequence of nucleotides 400-422, 1430-1452, 1863-1885, 410-432, 518-540, 519-541, 640-662, or 1864-1886 of SEQ ID NO: 1;
c) wherein the sense strand comprises at least 15 contiguous nucleotides differing by no more than 3 nucleotides from the nucleotide sequence of any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, or 55, and the antisense strand comprises at least 15 contiguous nucleotides differing by no more than 3 nucleotides from the corresponding portion of the nucleotide sequence of any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, or 56;
d) wherein the antisense strand comprises a region of complementarity to an mRNA encoding the target gene, and wherein the region of complementarity comprises at least 15 contiguous nucleotides differing by no more than 3 nucleotides from any one of the antisense nucleotide sequences in any one of Tables 2-17, 19 and 20.

2.-22. (canceled)

23. The dsRNA agent of claim 1, wherein at least one of the nucleotides of the dsRNA agent comprises a nucleotide modification.

24. (canceled)

25. The dsRNA agent of claim 1, wherein all of the nucleotides of the sense strand and all of the nucleotides of the antisense strand comprise a nucleotide modification.

26. The dsRNA agent of claim 23, wherein at least one of the nucleotide modifications is selected from the group consisting of a deoxy-nucleotide modification, a 3′-terminal deoxythimidine (dT) nucleotide modification, a 2′-O-methyl nucleotide modification, a 2′-fluoro nucleotide modification, a 2′-deoxy-nucleotide modification, a locked nucleotide modification, an unlocked nucleotide modification, a conformationally restricted nucleotide modification, a constrained ethyl nucleotide modification, an abasic nucleotide modification, a 2′-amino-nucleotide modification, a 2′-O-allyl-nucleotide modification, 2′-C-alkyl-nucleotide modification, 2′-hydroxly-nucleotide modification, a 2′-methoxyethyl nucleotide modification, a 2′-O-alkyl-nucleotide modification, a morpholino nucleotide modification, a phosphoramidate modification, a non-natural base comprising nucleotide modification, a tetrahydropyran nucleotide modification, a 1,5-anhydrohexitol nucleotide modification, a cyclohexenyl nucleotide modification, a nucleotide comprising a phosphorothioate group modification, a nucleotide comprising a methylphosphonate group modification, a nucleotide comprising a 5′-phosphate modification, a nucleotide comprising a 5′-phosphate mimic modification, a thermally destabilizing nucleotide modification, a glycol nucleotide (GNA) modification, a nucleotide comprising a 2′ phosphate modification, and a 2-O—(N-methylacetamide) nucleotide modification; and combinations thereof.

27.-30. (canceled)

31. The dsRNA agent of claim 1, further comprising a phosphate or phosphate mimic at the 5′-end of the antisense strand.

32. (canceled)

33. The dsRNA agent of claim 1, wherein the 3′ end of the sense strand is protected via an end cap which is a cyclic group having an amine, said cyclic group being selected from the group consisting of pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl.

34. The dsRNA agent of claim 1, wherein the double stranded region is 19-30 nucleotide pairs in length.

35.-38. (canceled)

39. The dsRNA agent of claim 1, wherein each strand is independently no more than 30 nucleotides in length.

40.-43. (canceled)

44. The dsRNA agent of claim 1, wherein at least one strand comprises a 3′ overhang of at least 1 nucleotide; or at least one strand comprises a 3′ overhang of at least 2 nucleotides.

45. (canceled)

46. The dsRNA agent of claim 1, wherein one or more C22 hydrocarbon chains is conjugated to one or more internal positions on at least one strand.

47.-69. (canceled)

70. The dsRNA agent of claim 1, further comprising a targeting ligand that targets a receptor which mediates delivery to adipose tissue.

71. (canceled)

72. The dsRNA agent of claim 1, further comprising a targeting ligand that targets a liver tissue.

73. The dsRNA agent of claim 72, wherein the targeting ligand is conjugated to the 3′ end of the sense strand of the dsRNA agent.

74. The dsRNA agent of claim 72, wherein the targeting ligand is an N-acetylgalactosamine (GalNAc) derivative.

75. The dsRNA agent of claim 72, wherein the targeting ligand is one or more GalNAc derivatives attached through a monovalent, bivalent, or trivalent branched linker.

76. The dsRNA agent of claim 72, wherein the targeting ligand is

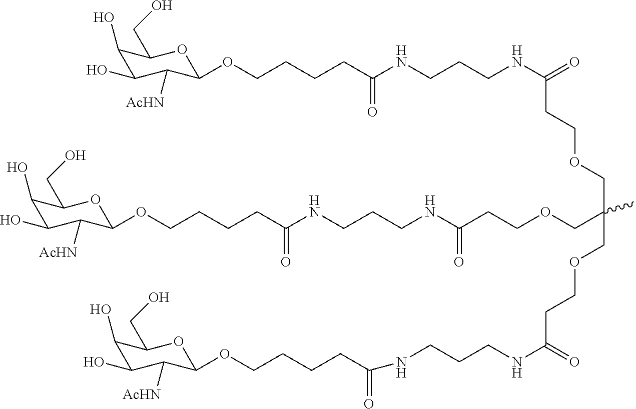 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

77. The dsRNA agent of claim 76, wherein the dsRNA agent is conjugated to the targeting ligand as shown in the following schematic 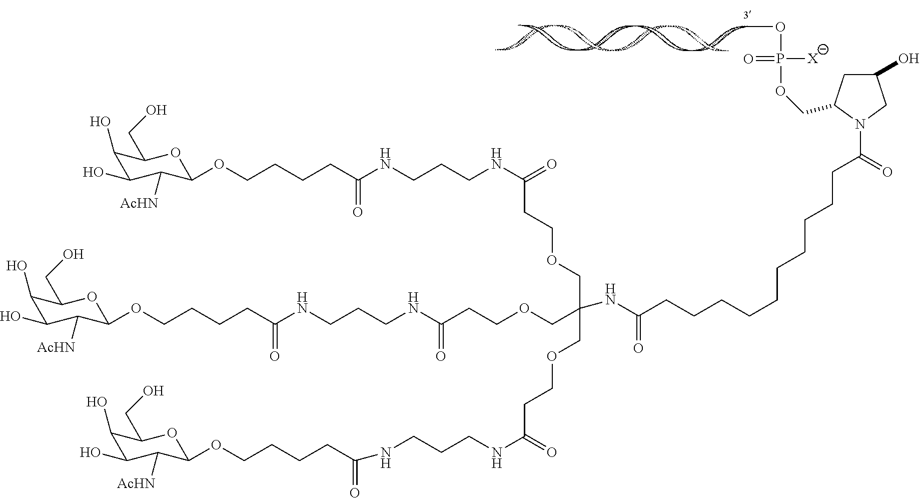
and, wherein X is O or S.

78. The dsRNA agent of claim 77, wherein the X is O.

79. (canceled)

80. The dsRNA agent of claim 1, wherein the dsRNA agent further comprises at least one phosphorothioate or methylphosphonate internucleotide linkage.

81.-121. (canceled)

122. A cell containing the dsRNA agent of claim 1.

123. A pharmaceutical composition for inhibiting expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC) comprising the dsRNA agent of claim 1 and a pharmaceutically acceptable carrier.

124.-128. (canceled)

129. A method of inhibiting expression of a metabolic disorder-associated target gene selected from the group consisting of inhibin subunit beta E (INHBE), activin A receptor type 1C (ACVR1C), perilipin-1 (PLIN1), phosphodiesterase 3B (PDE3B), and inhibin subunit beta C (INHBC) in a cell, the method comprising contacting the cell with the dsRNA agent of claim 1, thereby inhibiting expression of the metabolic disorder-associated target gene in the cell.

130.-144. (canceled)

145. A method of treating a subject having a metabolic disorder, comprising administering to the subject a therapeutically effective amount of the dsRNA agent of claim 1, thereby treating the subject having the metabolic disorder.

146.-169. (canceled)

170. A kit, a vial, or a syringe comprising the dsRNA agent of claim 1.

171.-173. (canceled)
